(19) United States
(12) Patent Application Publication
(10) Pub. No.: US 2024/0309003 A1
(43) Pub. Date: Sep. 19, 2024

(54) HETEROCYCLIC COMPOUNDS AS TRIGGERING RECEPTOR EXPRESSED ON MYELOID CELLS 2 AGONISTS AND METHODS OF USE

(71) Applicant: Vigil Neuroscience, Inc., Watertown, MA (US)

(72) Inventors: Jonathan B. HOUZE, Watertown, MA (US); Maxence BOS, Saint-Laurent (CA); John MANCUSO, Saint-Laurent (CA); Ivan FRANZONI, Saint-Laurent (CA); Bhaumik PANDYA, Watertown, MA (US); Alan KAPLAN, Watertown, MA (US)

(73) Assignee: Vigil Neuroscience, Inc., Watertown, MA (US)

(21) Appl. No.: 18/558,504

(22) PCT Filed: May 4, 2022
(86) PCT No.: PCT/US2022/072095

§ 371 Date: Nov. 1, 2023

Related U.S. Application Data
(60) Provisional application No. 63/201,531, filed on May 4, 2021.
(60) Provisional application No. 63/263,811, filed on Nov. 9, 2021.

(51) Int. Cl. C07D 475/00; A61K 31/438; A61K 31/444; A61K 31/498; A61K 31/4985; A61K 31/519; A61K 31/5377; A61K 31/541; A61K 31/553; C07D 471/04; C07D 491/147; C07D 519/00

(52) U.S. Cl. CPC C07D 475/00; A61K 31/438; A61K 31/444; A61K 31/498; A61K 31/4985; A61K 31/519; A61K 31/5377; A61K 31/541; A61K 31/553; C07D 471/04; C07D 491/147; C07D 519/00

(57) ABSTRACT

The present disclosure provides compounds of Formula I, useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

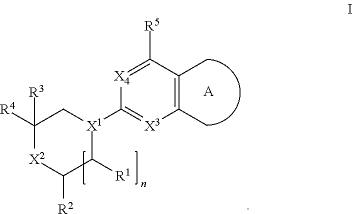

### CROSS REFERENCE TO RELATED APPLICATIONS

This application claims the priority of U.S. provisional application No. 63/201,531 filed May 4, 2021 and U.S. provisional application No. 63/263,811 filed Nov. 9, 2021, each of which is hereby incorporated by reference in its entirety.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neuropathology, 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016; Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”), Toll-like receptors (“TLR”), Fc receptors, nucleotide-binding oligomerization domain (“NOD”) and purinergic G protein-coupled receptors. Doens and Fernandez 2014, Madry and Attwell 2015, Hickman and El Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med, 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010; Jay et al. Mol Neurodegener, 2017). TREM2/DAP12 complexes in particular act as a signaling unit that can be characterized as pro-activation on microglial phenotypes in addition to peripheral macrophages and osteoclasts (Otero et al. J Immunol, 2012; Kobayashi et al. J Neurosci, 2016; Jaitin et al., Cell, 2019. In the CNS, signaling through TREM2 has been studied in the context of ligands such as phospholipids, cellular debris, apolipoproteins, and myelin (Wang et al. Cell, 2015; Kober and Brett, J Mol Biol, 2017; Shirotani et al., Sci Rep, 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol, 2015, Wu et al., Mol Brain, 2017).

Coding variants in the TREM2 locus has been associated with late onset Alzheimer's disease (“LOAD”) in human genome-wide association studies, linking a loss-of-receptor function to a gain in disease risk (Jonsson et al. N Engl J Med 2013, Sims et al. Nat Genet 2017). Genetic variation of other genes selectively expressed by microglia in the CNS, for example, CD33, PLCg2 and MS4A4A/6A have reached genome-wide significance for their association with LOAD risk (Hollingworth et al. Nat Genet 2011, Sims et al. Nat Genet 2017, Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM2 in LOAD, homozygous loss-of-function mutations in TREM2 are causal for an early onset dementia syndrome known as Polycystic lipomembranous osteodysplasia with sclerosing leukoencephalopathy (“PLOSL”) or Nasu-Hakola disease (“NHD”) (Golde et al. Alzheimers Res Ther 2013, Dardiotis et al. Neurobiol Aging 2017). This progressive neurodegenerative disease typically manifests in the 3^{rd} decade of life and is pathologically characterized by loss of myelin in the brain concomitant with gliosis, unresolved neuroinflammation, and cerebral atrophy. Typical neuropsychiatric presentations are often preceded by osseous abnormalities, such as bone cysts and loss of peripheral bone density (Bianchin et al. Cell Mol Neurobiol 2004; Madry et al. Clin Orthop Relat Res 2007, Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003, Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R⁴⁷H loss-of-function mutant transgenic mice (Ulland et al. Cell, 2017, Kang et al. Hum Mol Genet 2018). Although unable to recapitulate the neurological manifestations of PLOSL, TREM2 KO mice show abnormalities in bone ultrastructure (Otero et al. J Immunol 2012). When the TREM2 KO or mutant mice have been crossed onto familial Alzheimer's disease transgenic mouse background such as the 5XFAD amyloidogenic mutation lines, marked phenotypes have been observed (Ulrich et al. Neuron, 2017). These in vivo phenotypes of TREM2 loss-of-function in the CNS include elevated the plaque burden and lower levels of secreted microglial factors SPP1 and Osteopontin that are characteristic of the microglial response to amyloid pathology (Ulland et al. Cell, 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios, 5XFAD familial Alzheimer's disease model mice, and Amyotrophic Lateral Sclerosis SOD1 mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5XFAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM2 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases, such as Alzheimer's disease.

### SUMMARY

First, provided herein is a compound of Formula I″

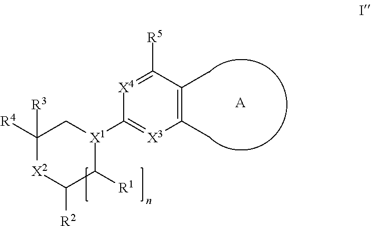

or a tautomer thereof, or a pharmaceutically acceptable salt of said compound or said tautomer, wherein
Ring A together with the 6-membered ring system to which it is fused forms a bicyclic ring system of formula

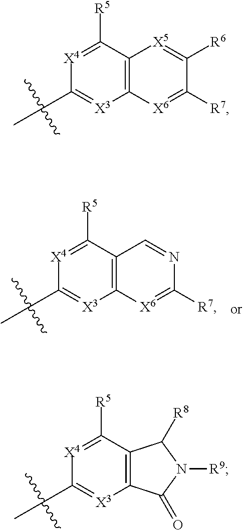

wherein
X¹ is CH, C(OH), C(OCH₃), CF, or N;
X² is CH₂, CHF, CF₂, (C═O), O, S(O)₂, or NH;
X³ is CH or N;
X⁴ is CH or N;
X⁵ is CH or N;
X⁶ is CH or N;
R¹ is H, C₁₋₃alkyl, or CH₂OH;
R² is H, C₁₋₃alkyl, C₁₋₆haloalkyl, or C₃₋₆cycloalkyl;
R³ is H or C₁₋₃alkyl;
or R¹ and R³ are taken together with their intervening atoms to form a cyclic group selected from a 3-8 membered saturated or partially unsaturated monocyclic heterocyclic ring;
R⁴ is C₁₋₆alkyl, C₁₋₆haloalkyl, diC₁₋₃alkylamino, —C(═O)O(C₁₋₆alkyl), —C(═O)(heteroaryl), C₃₋₆cycloalkyl, C₃₋₆heterocycloalkyl, phenyl, 5-membered heteroaryl, or 6-membered heteroaryl; wherein
(1) the C₁₋₆alkyl, C₃₋₆cycloalkyl, or C₃₋₆heterocycloalkyl is optionally substituted with 1 to 6 substituents independently selected from C═O, C(═O)CH₃, —OH, C₁₋₆haloalkyl, 5-membered heteroaryl, and C(═O)OCH₂-phenyl;
(2) the phenyl, 5-membered heteroaryl, 6-membered heteroaryl, or —C(═O)(heteroaryl) is optionally substituted with 1 to 3 substituents independently selected from halogen, CD₃, C₁₋₆alkyl, C₁₋₆haloalkyl, C₁₋₆alkoxy, C₁₋₆haloalkoxy, —(C₁₋₃alkyl)O(C₁₋₃alkyl), CH₂OH, —CN, C₂₋₄alkenyl, C₃₋₆cycloalkyl, and C₃₋₆heterocycloalkyl; wherein
   the C₁₋₆alkyl, C₁₋₆haloalkyl, and C₃₋₆cycloalkyl of subsection (2) are optionally substituted with 1 to 6 substituents independently selected from halogen, C₁₋₆alkyl, C₁₋₆alkoxy, OH, C₃₋₆cycloalkyl, N(CH₃)C(═O)CH₃, or phenyl, wherein the phenyl is optionally substituted with 1 to 6 substitutents independently selected from halogen, C₁₋₆alkyl, and C₁₋₆alkoxy; and wherein one or more of C₁₋₆alkyl are taken together with their intervening atoms to form a C₃₋₆cycloalkyl;
   the C₃₋₆heterocycloalkyl of subsection (2) is optionally substituted with 1 to 3 substituents selected from halogen, C₁₋₃alkyl, and —C(═O)O(C₁₋₆alkyl);
   the 5-membered heteroaryl of subsection (1) is optionally substituted with 1 to 3 substituents selected from halogen and C₃₋₆cycloalkyl;
R⁵ is C₁₋₆alkyl, C₂₋₆alkenyl, C₂₋₆alkynyl, C₁₋₆haloalkyl, C₃₋₆cycloalkyl, C₃₋₆cycloalkenyl, C₅₋₈spiroalkyl, C₅₋₈tricycloalkyl, cyclopent-1-en-1-yl, cyclohex-1-en-1-yl, phenyl, 5-membered heteroaryl, 6-membered heteroaryl, aziridine-1-yl, spiro[3.3]heptane-6-yl, pyrrolidine-1-yl, 3-azabicyclo[3.1.0]hexan-3-yl, morpholine-4-yl, piperidine-1-yl, benzothiazole-5-yl, dihydro-indene-5-yl, bicyclo[4.2.0]octa-1(6),2,4-triene-3-yl, or —OCH₂—(C₃₋₆cycloalkyl),
   wherein the C₁₋₆alkyl, C₂₋₆alkenyl, C₂₋₆alkynyl, C₃₋₆cycloalkyl, C₃₋₆cycloalkenyl, C₅₋₈spiroalkyl, C₅₋₈tricycloalkyl, spiro[3.3]heptane-6-yl, cyclopent-1-en-1-yl, cyclohex-1-en-1-yl, phenyl, 5-membered heteroaryl, and 6-membered heteroaryl are further optionally substituted with 1 to 4 substituents independently selected from deuterium, halogen, C₁₋₃alkyl, C₁₋₃haloalkyl, C₁₋₃alkoxy, C₁₋₃haloalkoxy, and C₃₋₆cycloalkyl; and
   wherein the aziridine-1-yl, pyrrolidine-1-yl, 3-azabicyclo[3.1.0]hexan-3-yl, morpholine-4-yl, piperidine-1-yl, 2-benzothiazole-5-yl, and —OCH₂—(C₃₋₆cycloalkyl) are further optionally substituted with 1 to 4 substituents independently selected from halogen, C₁₋₃alkyl, C₁₋₃haloalkyl, C₁₋₃alkoxy, 5-membered heteroaryl, and C₁₋₃haloalkoxy; and
      wherein the 5-membered heteroaryl is further substituted with C₃₋₆cycloalkyl,
      wherein the C₁₋₃alkyl is further optionally substituted with 1 to 4 substituents independently selected from halogen or —CN; and
      wherein one or more of C₁₋₆alkyl, C₂₋₆alkenyl, C₂₋₆alkynyl and C₁₋₆haloalkyl are taken together with their intervening atoms to form a C₃₋₆cycloalkyl optionally substituted with 1 to 4 substituents independently selected from halogen, C₁₋₃alkyl;
R⁶ is H, halogen, CD₃, C₁₋₃alkyl, CH₂CN, C(═O)NH₂, C(═O)NC(CH₃)₂, C₂₋₄alkoxy, C₁₋₆haloalkyl, C₁₋₆haloalkoxy, or C₃₋₆cycloalkyl;
R⁷ is H, halogen, CD₃, C₁₋₃alkyl, C₁₋₆haloalkyl, or C₃₋₆cycloalkyl;
R⁸ is H or C₁₋₃alkyl;
R⁹ is H or C₁₋₅alkyl; and
n is 0, 1, or 2; provided that when X¹ is N and n is 0, X² is not NH or O.

Second, provided herein is a pharmaceutical composition comprising a compound of Formula I″, or a tautomer thereof, or a pharmaceutically acceptable salt of said compound or said tautomer, and a pharmaceutically acceptable excipient.

Third, provided herein is a compound of Formula I″, or a tautomer thereof, or a pharmaceutically acceptable salt of said compound or said tautomer, or a pharmaceutical composition as described hereinabove, for use in treating or preventing a condition associated with a loss of function of human TREM2.

Fourth, provided herein is a compound of Formula I″, or a tautomer thereof, or a pharmaceutically acceptable salt of said compound or said tautomer, or a pharmaceutical composition described hereinabove, for use in treating or preventing Parkinson's disease, rheumatoid arthritis, Alzheimer's disease, Nasu-Hakola disease, frontotemporal dementia, multiple sclerosis, prion disease, or stroke.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein is a compound of Formula I′

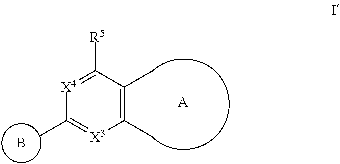

or a tautomer thereof, or a pharmaceutically acceptable salt of said compound or said tautomer, wherein
Ring A together with the 6-membered ring system to which it is fused forms a bicyclic ring system of formula

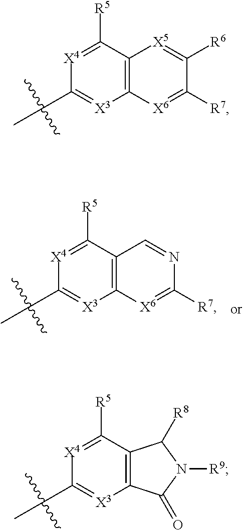

Ring B is

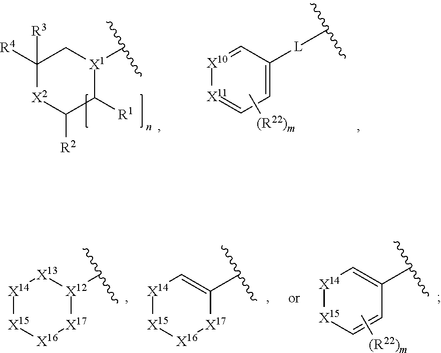

X¹ is CH or N;
X² is CH₂, CHF, CF₂, O, or NH;
X³ is CR¹⁸, CH or N;
X⁴ is CR¹⁹, CH or N;
X⁵ is CR²⁰, CH or N;
X⁶ is CR²¹, CH or N;
R¹ is H or C₁₋₃alkyl;
R² is H or C₁₋₃alkyl;
R³ is H or C₁₋₃alkyl;
R⁴ is C₁₋₆alkyl, C₁₋₆haloalkyl, diC₁₋₃alkylamino, —C(═O)O(C₁₋₆alkyl), C₃₋₆cycloalkyl, C₃₋₆heterocycloalkyl, phenyl, 5-membered heteroaryl, or 6-membered heteroaryl; wherein
(1) the C₃₋₆cycloalkyl or the C₃₋₆heterocycloalkyl is optionally substituted with C═O;
(2) the phenyl, 5-membered heteroaryl, or 6-membered heteroaryl group is optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein
   the C₁₋₆alkyl and C₁₋₆haloalkyl of subsection (2) are optionally substituted with OH; and wherein
   the C₃₋₆heterocycloalkyl of subsection (2) is optionally substituted with 1 to 3 substituents selected from halogen, C₁₋₃alkyl, and —C(═O)O(C₁₋₆alkyl);
R⁵ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁶ and R⁷ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁶ and R⁷ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R⁸ is H or C₁₋₃alkyl;
R⁹ is H or C₁₋₅alkyl;
n is 0 or 1; provided that when X¹ is N and n is 0, X² is not NH or O;
L is a bond or an optionally substituted straight chain or branched C₁₋₆ alkylene;
X¹⁰ is CH, N or CR¹⁰;
X¹¹ is CH, N or CR¹¹;
provided that when one of X¹⁰ or X¹¹ is N, the other is not N;
R¹⁰ and R¹¹ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R¹⁰ and R¹¹ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹² is N, CH, or CR¹²;
X¹³ is O, NR¹³, C(R¹³)₂, CHR¹³, SO₂, or C═O;
X¹⁴ is O, NR¹⁴, C(R¹⁴)₂, CHR¹⁴, SO₂, or C═O;
X¹⁵ is O, NR¹⁵, C(R¹⁵)₂, CHR¹⁵, SO₂, or C═O;
X¹⁶ is O, NR¹⁶, C(R¹⁶)₂, CHR¹⁶, SO₂, or C═O;
X¹⁷ is a direct bond, O, NR¹⁷, C(R¹⁷)₂, CHR¹⁷, —CH₂CH₂—, —OCH₂—, SO₂, or C═O;
R¹² is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R¹³, R¹⁴, R¹, R¹⁶, and R¹⁷ is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R², R¹³, R¹⁴, R¹⁵, R¹⁶, and R¹⁷ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹⁸, R¹⁹, R²⁰, and R²¹ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R²² is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Further provided herein is a compound of Formula I″

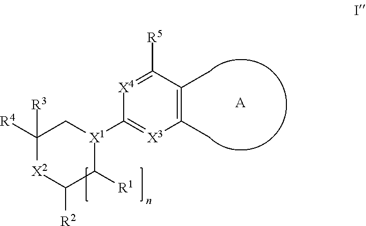

or a tautomer thereof, or a pharmaceutically acceptable salt of said compound or said tautomer, wherein
Ring A together with the 6-membered ring system to which it is fused forms a bicyclic ring system of formula

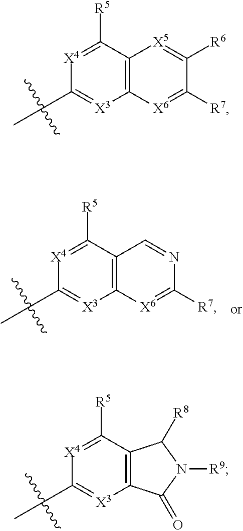

wherein
X¹ is CH, C(OH), C(OCH₃), CF, or N;
X² is CH₂, CHF, CF₂, (C═O), O, S(O)₂, or NH;
X³ is CH or N;
X⁴ is CH or N;
X⁵ is CH or N;
X⁶ is CH or N;
R¹ is H, C₁₋₃alkyl, or CH₂OH;
R² is H, C₁₋₃alkyl, C₁₋₆haloalkyl, or C₃₋₆cycloalkyl;
R³ is H or C₁₋₃alkyl;
or R¹ and R³ are taken together with their intervening atoms to form a cyclic group selected from a 3-8 membered saturated or partially unsaturated monocyclic heterocyclic ring;
R⁴ is C₁₋₆alkyl, C₁₋₆haloalkyl, diC₁₋₃alkylamino, —C(═O)O(C₁₋₆alkyl), —C(═O)(heteroaryl), C₃₋₆cycloalkyl, C₃₋₆heterocycloalkyl, phenyl, 5-membered heteroaryl, or 6-membered heteroaryl; wherein
(1) the C₁₋₆alkyl, C₃₋₆cycloalkyl, or C₃₋₆heterocycloalkyl is optionally substituted with 1 to 6 substituents independently selected from C═O, C(═O)CH₃, —OH, C₁₋₆haloalkyl, 5-membered heteroaryl, and C(═O)OCH₂-phenyl;
(2) the phenyl, 5-membered heteroaryl, 6-membered heteroaryl, or —C(═O)(heteroaryl) is optionally substituted with 1 to 3 substituents independently selected from halogen, CD₃, C₁₋₆alkyl, C₁₋₆haloalkyl, C₁₋₆alkoxy, C₁₋₆haloalkoxy, —(C₁₋₃alkyl)O(C₁₋₃alkyl), CH₂OH, —CN, C₂₋₄alkenyl, C₃₋₆cycloalkyl, and C₃₋₆heterocycloalkyl; wherein
   the C₁₋₆alkyl, C₁₋₆haloalkyl, and C₃₋₆cycloalkyl of subsection (2) are optionally substituted with 1 to 6 substituents independently selected from halogen, C₁₋₆alkyl, C₁₋₆alkoxy, OH, C₃₋₆cycloalkyl, N(CH₃)C(═O)CH₃, or phenyl, wherein the phenyl is optionaly substituted with 1 to 6 substitutents independently selected from halogen, C₁₋₆alkyl, and C₁₋₆alkoxy; and wherein one or more of C₁₋₆alkyl are taken together with their intervening atoms to form a C₃₋₆cycloalkyl;
   the C₃₋₆heterocycloalkyl of subsection (2) is optionally substituted with 1 to 3 substituents selected from halogen, C₁₋₃alkyl, and —C(═O)O(C₁₋₆alkyl);
   the 5-membered heteroaryl of subsection (1) is optionally substituted with 1 to 3 substituents selected from halogen and C₃₋₆cycloalkyl;
R⁵ is C₁₋₆alkyl, C₂₋₆alkenyl, C₂₋₆alkynyl, C₁₋₆haloalkyl, C₃₋₆cycloalkyl, C₃₋₆cycloalkenyl, C₅₋₈spiroalkyl, C₅₋₈tricycloalkyl, cyclopent-1-en-1-yl, cyclohex-1-en-1-yl, phenyl, 5-membered heteroaryl, 6-membered heteroaryl, aziridine-1-yl, spiro[3.3]heptane-6-yl, pyrrolidine-1-yl, 3-azabicyclo[3.1.0]hexan-3-yl, morpholine-4-yl, piperidine-1-yl, benzothiazole-5-yl, dihydro-indene-5-yl, bicyclo[4.2.0]octa-1(6),2,4-triene-3-yl, or —OCH₂—(C₃₋₆cycloalkyl),
   wherein the C₁₋₆alkyl, C₂₋₆alkenyl, C₂₋₆alkynyl, C₃₋₆cycloalkyl, C₃₋₆cycloalkenyl, C₅₋₈spiroalkyl, C₅₋₈tricycloalkyl, spiro[3.3]heptane-6-yl, cyclopent-1-en-1-yl, cyclohex-1-en-1-yl, phenyl, 5-membered heteroaryl, and 6-membered heteroaryl are further optionally substituted with 1 to 4 substituents independently selected from deuterium, halogen, C₁₋₃alkyl, C₁₋₃haloalkyl, C₁₋₃alkoxy, C₁₋₃haloalkoxy, and C₃₋₆cycloalkyl; and
   wherein the aziridine-1-yl, pyrrolidine-1-yl, 3-azabicyclo[3.1.0]hexan-3-yl, morpholine-4-yl, piperidine-1-yl, 2-benzothiazole-5-yl, and —OCH₂—(C₃₋₆cycloalkyl) are further optionally substituted with 1 to 4 substituents independently selected from halogen, C₁₋₃alkyl, C₁₋₃haloalkyl, C₁₋₃alkoxy, 5-membered heteroaryl, and C₁₋₃haloalkoxy; and
      wherein the 5-membered heteroaryl is further substituted with C₃₋₆cycloalkyl,
      wherein the C₁₋₃alkyl is further optionally substituted with 1 to 4 substituents independently selected from halogen or —CN; and
      wherein one or more of C₁₋₆alkyl, C₂₋₆alkenyl, C₂₋₆alkynyl and C₁₋₆haloalkyl are taken together with their intervening atoms to form a C₃₋₆cycloalkyl optionally substituted with 1 to 4 substituents independently selected from halogen, C₁₋₃alkyl;
R⁶ is H, halogen, CD₃, C₁₋₃alkyl, CH₂CN, C(═O)NH₂, C(═O)NC(CH₃)₂, C₂₋₄alkoxy, C₁₋₆haloalkyl, C₁₋₆haloalkoxy, or C₃₋₆cycloalkyl;
R⁷ is H, halogen, CD₃, C₁₋₃alkyl, C₁₋₆haloalkyl, or C₃₋₆cycloalkyl;
R⁸ is H or C₁₋₃alkyl;
R⁹ is H or C₁₋₅alkyl; and
n is 0, 1, or 2; provided that when X¹ is N and n is 0, X² is not NH or O.

Further provided herein is a compound of Formula I

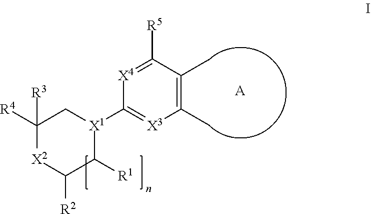

or a tautomer thereof, or a pharmaceutically acceptable salt of said compound or said tautomer, wherein
Ring A together with the 6-membered ring system to which it is fused forms a bicyclic ring system of formula

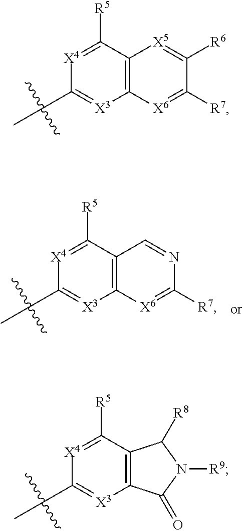

wherein
X¹ is CH or N;
X² is CH₂, CHF, CF₂, O, or NH;
X³ is CH or N;
X⁴ is CH or N;
X⁵ is CH or N;
X⁶ is CH or N;
R¹ is H or C₁₋₃alkyl;
R² is H or C₁₋₃alkyl;
R³ is H or C₁₋₃alkyl;
R⁴ is C₁₋₆alkyl, C₁₋₆haloalkyl, diC₁₋₃alkylamino, —C(═O)O(C₁₋₆alkyl), C₃₋₆cycloalkyl, C₃₋₆heterocycloalkyl, phenyl, 5-membered heteroaryl, or 6-membered heteroaryl; wherein
(1) the C₃₋₆cycloalkyl or the C₃₋₆heterocycloalkyl is optionally substituted with C═O;
(2) the phenyl, 5-membered heteroaryl, or 6-membered heteroaryl group is optionally substituted with 1 to 3 substituents independently selected from halogen, C₁₋₆alkyl, C₁₋₆haloalkyl, C_{1-6 6}alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein
   the C₁₋₆alkyl and C₁₋₆haloalkyl of subsection (2) are optionally substituted with OH; and wherein
   the C₃₋₆heterocycloalkyl of subsection (2) is optionally substituted with 1 to 3 substituents selected from halogen, C₁₋₃alkyl, and —C(═O)O(C₁₋₆alkyl);
R⁵ is C₁₋₆alkyl, C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, 4-methylbenzo[1,3]dioxolyl, 5-methylbenzo[1,3]dioxolyl, piperidine-1-yl, or —OCH₂—(C₃₋₆cycloalkyl),
   wherein the C₁₋₆alkyl, C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, C₁₋₃alkoxy, and C₁₋₃haloalkoxy;
R⁶ is H, halogen, C₁₋₃alkyl, C₁₋₆haloalkoxy, —(C₁₋₃alkyl)O(C₁₋₃alkyl)(C₃₋₆cycloalkyl);
R⁷ is H, halogen, or C₁₋₃alkyl;
R⁸ is H or C₁₋₃alkyl;
R⁹ is H or C₁₋₅alkyl; and
n is 0 or 1; provided that when X¹ is N and n is 0, X² is not NH or O.

In some embodiments, the compound is not:
4-(3-fluoro-1-azetidinyl)-6,7-dimethyl-2-((2S)-2-(1-methyl-1H-pyrazol-4-yl)-4-morpholinyl)pteridine;
4-(3,3-difluoro-1-piperidinyl)-6,7-dimethyl-2-((2S)-2-(1-methyl-1H-pyrazol-4-yl)-4-morpholinyl)pteridine;
2-((2S)-2-(1-cyclopropyl-1H-pyrazol-4-yl)-4-morpholinyl)-7-methyl-4-(3-(trifluoromethyl)bicyclo[1.1.1]pentan-1-yl)pyrido[2,3-d]pyrimidine;
6,7-dimethyl-2-((2S)-2-(1-methyl-1H-pyrazol-4-yl)-4-morpholinyl)-4-((cis-3-(trifluoromethyl)cyclobutyl)methoxy)pyrido[2,3-d]pyrimidine; or
2-methyl-6-((2S)-2-(1-methyl-1H-pyrazol-4-yl)-4-morpholinyl)-4-(cis-3-(trifluoromethyl)cyclobutyl)-2,3-dihydro-1H-pyrrolo[3,4-c]pyridin-1-one.

As defined generally above, Ring B is

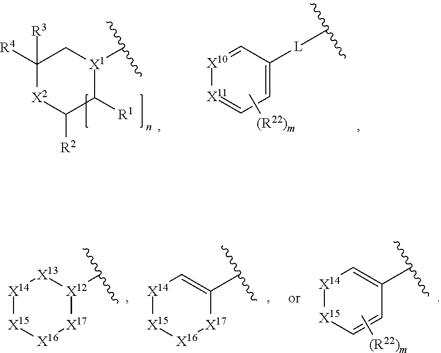

In some embodiments, Ring B is

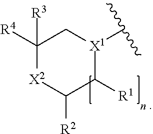

Further provided herein is a compound of Formula I′″

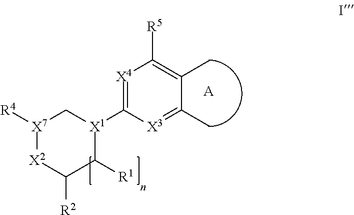

or a tautomer thereof, or a pharmaceutically acceptable salt of said compound or said tautomer, wherein
Ring A together with the 6-membered ring system to which it is fused forms a bicyclic ring system of formula

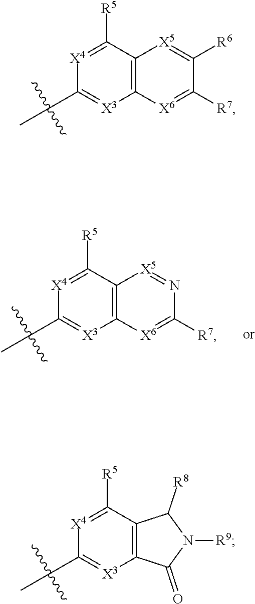

X¹ is CH, C(OH), C(OCH₃), CF, or N;
X² is CH₂, CHF, CF₂, (C═O), O, S(O)₂, or NH;
X³ is CH or N;
X⁴ is CH or N;
X⁵ is CH or N;
X⁶ is CH or N;
X⁷ is CH or N;
R¹ is H, C₁₋₃alkyl, or CH₂OH;
R² is H, C₁₋₃alkyl, C₁₋₆haloalkyl, or C₃₋₆cycloalkyl;
R³ is H or C₁₋₃alkyl;
or R¹ and R³ are taken together with their intervening atoms to form a cyclic group selected from a 3-8 membered saturated or partially unsaturated monocyclic heterocyclic ring;
R⁴ is C₁₋₆alkyl, C₁₋₆haloalkyl, diC₁₋₃alkylamino, —C(═O)O(C₁₋₆alkyl), —C(═O)(heteroaryl), C₃₋₆cycloalkyl, C₃₋₆heterocycloalkyl, phenyl, 5-membered heteroaryl, or 6-membered heteroaryl; wherein
(1) the C₁₋₆alkyl, C₃₋₆cycloalkyl, or C₃₋₆heterocycloalkyl is optionally substituted with 1 to 6 substituents independently selected from C═O, C(═O)CH₃, —OH, C₁₋₆haloalkyl, 5-membered heteroaryl, and C(═O)OCH₂-phenyl;
(2) the phenyl, 5-membered heteroaryl, 6-membered heteroaryl, or —C(═O)(heteroaryl) is optionally substituted with 1 to 3 substituents independently selected from halogen, CD₃, C₁₋₆alkyl, C-₆haloalkyl, C₁₋₆alkoxy, C₁₋₆haloalkoxy, —(C₁₋₃alkyl)O(C₁₋₃alkyl), CH₂OH, —CN, C₂₋₄alkenyl, C₃₋₆cycloalkyl, and C₃₋₆heterocycloalkyl; wherein
   the C₁₋₆alkyl, C₁₋₆haloalkyl, and C₃₋₆cycloalkyl of subsection (2) are optionally substituted with 1 to 6 substituents independently selected from halogen, C₁₋₆alkyl, C₁₋₆alkoxy, OH, C₃₋₆cycloalkyl, N(CH₃)C(═O)CH₃, or phenyl, wherein the phenyl is optionaly substituted with 1 to 6 substitutents independently selected from halogen, C₁₋₆alkyl, and C₁₋₆alkoxy; and wherein one or more of C₁₋₆alkyl are taken together with their intervening atoms to form a C₃₋₆cycloalkyl;
   the C₃₋₆heterocycloalkyl of subsection (2) is optionally substituted with 1 to 3 substituents selected from halogen, C₁₋₃alkyl, and —C(═O)O(C₁₋₆alkyl);
   the 5-membered heteroaryl of subsection (1) is optionally substituted with 1 to 3 substituents selected from halogen and C₃₋₆cycloalkyl;
R⁵ is C₁₋₆alkyl, C₂₋₆alkenyl, C₂₋₆alkynyl, C₁₋₆haloalkyl, C₃₋₆cycloalkyl, C₃₋₆cycloalkenyl, C₅₋₈spiroalkyl, C₅₋₈tricycloalkyl, cyclopent-1-en-1-yl, cyclohex-1-en-1-yl, phenyl, 5-membered heteroaryl, 6-membered heteroaryl, aziridine-1-yl, spiro[3.3]heptane-6-yl, pyrrolidine-1-yl, 3-azabicyclo[3.1.0]hexan-3-yl, morpholine-4-yl, piperidine-1-yl, benzothiazole-5-yl, dihydro-indene-5-yl, bicyclo[4.2.0]octa-1(6),2,4-triene-3-yl, or —OCH₂—(C₃₋₆cycloalkyl),
   wherein the C₁₋₆alkyl, C₂₋₆alkenyl, C₂₋₆alkynyl, C₃₋₆cycloalkyl, C₃₋₆cycloalkenyl, C₅₋₈spiroalkyl, C₅₋₈tricycloalkyl, spiro[3.3]heptane-6-yl, cyclopent-1-en-1-yl, cyclohex-1-en-1-yl, phenyl, 5-membered heteroaryl, and 6-membered heteroaryl are further optionally substituted with 1 to 4 substituents independently selected from deuterium, halogen, C₁₋₃alkyl, C₁₋₃haloalkyl, C₁₋₃alkoxy, C₁₋₃haloalkoxy, and C₃₋₆cycloalkyl; and
   wherein the aziridine-1-yl, pyrrolidine-1-yl, 3-azabicyclo[3.1.0]hexan-3-yl, morpholine-4-yl, piperidine-1-yl, 2-benzothiazole-5-yl, and —OCH₂—(C₃₋₆cycloalkyl) are further optionally substituted with 1 to 4 substituents independently selected from halogen, C₁₋₃alkyl, C₁₋₃haloalkyl, C₁₋₃alkoxy, 5-membered heteroaryl, and C₁₋₃haloalkoxy; and
      wherein the 5-membered heteroaryl is further substituted with C₃₋₆cycloalkyl,
      wherein the C₁₋₃alkyl is further optionally substituted with 1 to 4 substituents independently selected from halogen or —CN; and
      wherein one or more of C₁₋₆alkyl, C₂₋₆alkenyl, C₂₋₆alkynyl and C₁₋₆haloalkyl are taken together with their intervening atoms to form a C₃₋₆cycloalkyl optionally substituted with 1 to 4 substituents independently selected from halogen, C₁₋₃alkyl;
R⁶ is H, halogen, CD₃, C₁₋₃alkyl, CH₂CN, C(═O)NH₂, C(═O)NC(CH₃)₂, C₂₋₄alkoxy, C₁₋₆haloalkyl, C₁₋₆haloalkoxy, or C₃₋₆cycloalkyl;
R⁷ is H, halogen, CD₃, C₁₋₃alkyl, C₁₋₆haloalkyl, or C₃₋₆cycloalkyl;
R⁸ is H or C₁₋₃alkyl;
R⁹ is H or C₁₋₅alkyl; and
n is 0, 1, or 2; provided that when X¹ is N and n is 0, X² is not NH or O.

In some embodiments, the compound is a compound of Formula II

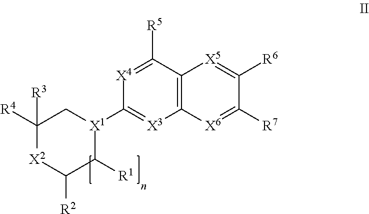

or a pharmaceutically acceptable salt thereof, wherein each variable is as defined above and described in embodiments herein both singly and in combination.

In some embodiments, the compound is a compound of Formula IIA

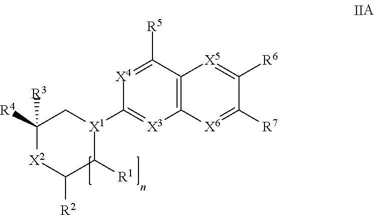

or a pharmaceutically acceptable salt thereof, wherein each variable is as defined above and described in embodiments herein both singly and in combination.

In some embodiments, the compound is a compound of Formula IIB

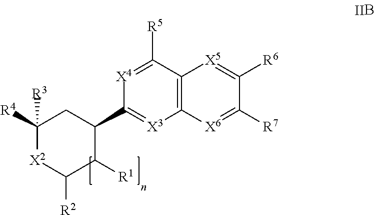

or a pharmaceutically acceptable salt thereof, wherein each variable is as defined above and described in embodiments herein both singly and in combination.

In some embodiments, the compound is a compound of Formula IIC

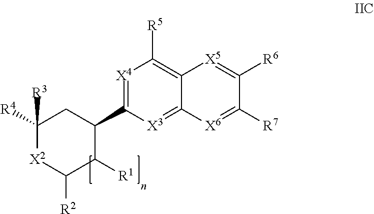

or a pharmaceutically acceptable salt thereof, wherein each variable is as defined above and described in embodiments herein both singly and in combination.

In some embodiments, the compound is a compound of Formula IID

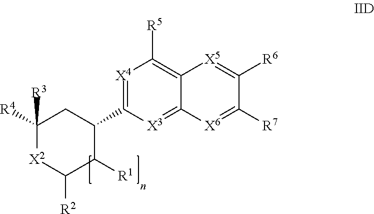

or a pharmaceutically acceptable salt thereof, wherein each variable is as defined above and described in embodiments herein both singly and in combination.

In some embodiments, the compound is a compound of Formula IIE

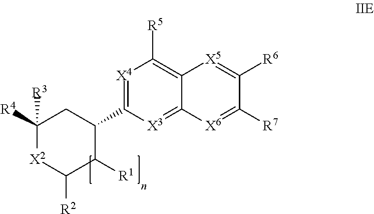

or a pharmaceutically acceptable salt thereof, wherein each variable is as defined above and described in embodiments herein both singly and in combination.

In some embodiments, the compound is a compound of Formula IIF

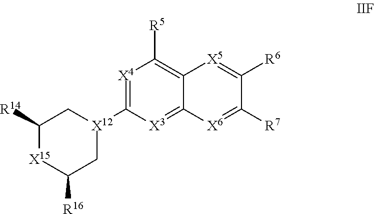

or a pharmaceutically acceptable salt thereof, wherein each variable is as defined above and described in embodiments herein both singly and in combination.

In some embodiments, the compound is a compound of Formula IIG

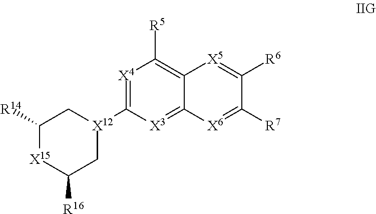

or a pharmaceutically acceptable salt thereof, wherein each variable is as defined above and described in embodiments herein both singly and in combination.

In some embodiments, the compound is a compound of Formula IIH

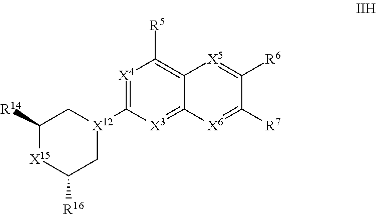

or a pharmaceutically acceptable salt thereof, wherein each variable is as defined above and described in embodiments herein both singly and in combination.

In some embodiments, the compound is a compound of Formula III

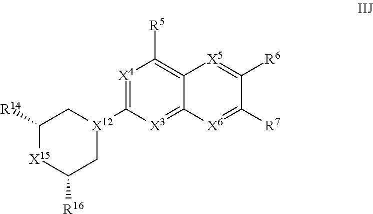

or a pharmaceutically acceptable salt thereof, wherein each variable is as defined above and described in embodiments herein both singly and in combination.

In some embodiments, the compound is a compound of Formula IIK

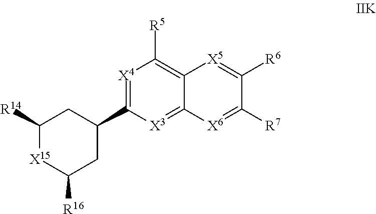

or a pharmaceutically acceptable salt thereof, wherein each variable is as defined above and described in embodiments herein both singly and in combination.

In some embodiments, the compound is a compound of Formula IIL

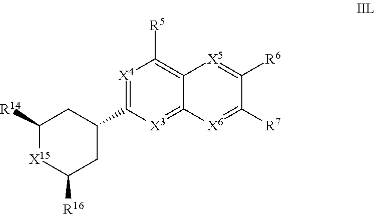

or a pharmaceutically acceptable salt thereof, wherein each variable is as defined above and described in embodiments herein both singly and in combination.

In some embodiments, the compound is a compound of Formula IIM

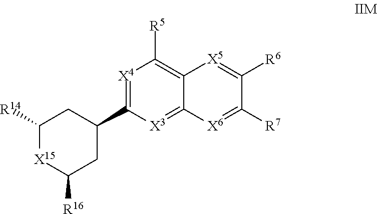

or a pharmaceutically acceptable salt thereof, wherein each variable is as defined above and described in embodiments herein both singly and in combination.

In some embodiments, the compound is a compound of Formula IIN

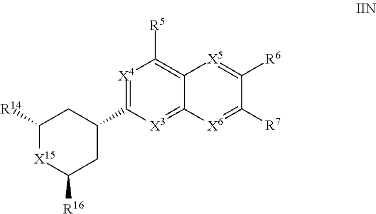

or a pharmaceutically acceptable salt thereof, wherein each variable is as defined above and described in embodiments herein both singly and in combination.

In some embodiments, the compound is a compound of Formula IIO

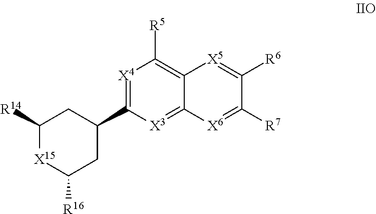

or a pharmaceutically acceptable salt thereof, wherein each variable is as defined above and described in embodiments herein both singly and in combination.

In some embodiments, the compound is a compound of Formula IIP

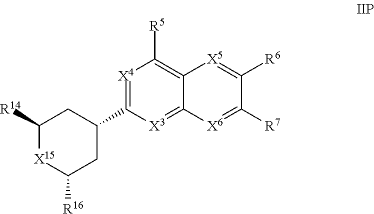

or a pharmaceutically acceptable salt thereof, wherein each variable is as defined above and described in embodiments herein both singly and in combination.

In some embodiments, the compound is a compound of Formula IIQ

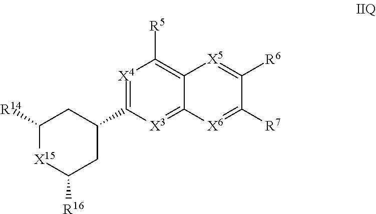

or a pharmaceutically acceptable salt thereof, wherein each variable is as defined above and described in embodiments herein both singly and in combination.

In some embodiments, the compound is a compound of Formula IIR

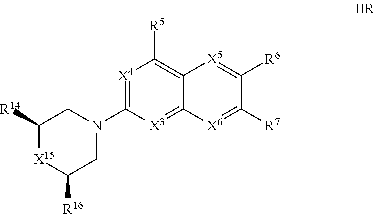

or a pharmaceutically acceptable salt thereof, wherein each variable is as defined above and described in embodiments herein both singly and in combination.

In some embodiments, the compound is a compound of Formula IIS

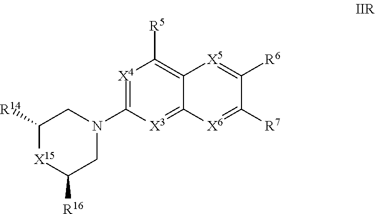

or a pharmaceutically acceptable salt thereof, wherein each variable is as defined above and described in embodiments herein both singly and in combination.

In some embodiments, the compound is a compound of Formula IIS

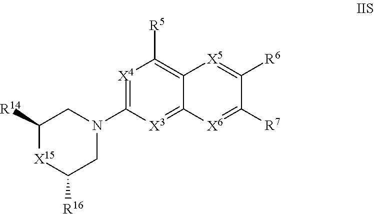

or a pharmaceutically acceptable salt thereof, wherein each variable is as defined above and described in embodiments herein both singly and in combination.

In some embodiments, the compound is a compound of Formula IIT

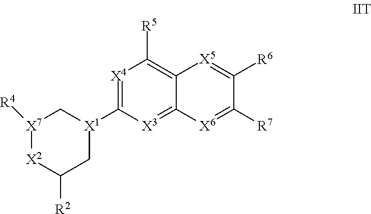

or a pharmaceutically acceptable salt thereof, wherein each variable is as defined above and described in embodiments herein both singly and in combination.

In some embodiments, the compound is a compound of Formula IIU

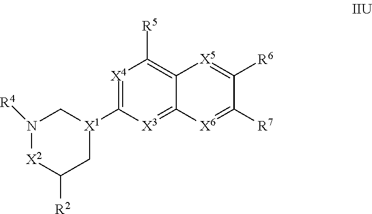

or a pharmaceutically acceptable salt thereof, wherein each variable is as defined above and described in embodiments herein both singly and in combination.

As defined generally above, X¹ is CH or N. In some embodiments, X¹ is CH. In some embodiments, X¹ is N. In some embodiments, X¹ is selected from those depicted in Table A below.

As defined generally above, X² is CH₂, CHF, CF₂, (C═O), O, S(O)₂, or NH. In some embodiments, X² is CH₂, CHF, CF₂, O, or NH. In some embodiments, X² is CH₂, CF₂, or O. In some embodiments, X² is O. In some embodiments, X² is (C═O) or S(O)₂. In some embodiments, X² is selected from those depicted in Table A below. In some embodiments, X² is selected from those depicted in Table A-2 below.

As defined generally above, X³ is CR¹⁸, CH or N. As defined generally above in Formula I, X³ is CH or N. In some embodiments, X³ is CH or N. In some embodiments, X³ is CH. In some embodiments, X³ is CR¹⁸. In some embodiments, X³ is N. In some embodiments, X³ is selected from those depicted in Table A below. In some embodiments, X³ is selected from those depicted in Table A-2 below.

As defined generally above, X⁴ is CR¹⁹, CH or N. As defined generally above in Formula I, X⁴ is CH or N. In some embodiments, X⁴ is CH or N. In some embodiments, X⁴ is CH. In some embodiments, X⁴ is CR¹⁹. In some embodiments, X⁴ is N. In some embodiments, X⁴ is selected from those depicted in Table A below. In some embodiments, X⁴ is selected from those depicted in Table A-2 below.

As defined generally above, X⁵ is CR²⁰, CH or N. As defined generally above in Formula I, X⁵ is CH or N. In some embodiments, X⁵ is CH or N. In some embodiments, X⁵ is CH. In some embodiments, X⁵ is CR²⁰. In some embodiments, X⁵ is N. In some embodiments, X⁵ is selected from those depicted in Table A below. In some embodiments, X⁵ is selected from those depicted in Table A-2 below.

As defined generally above, X⁶ is CR²¹, CH or N. As defined generally above in Formula I, X⁶ is CH or N. In some embodiments, X⁶ is CH or N. In some embodiments, X⁶ is CH. In some embodiments, X⁶ is CR²¹. In some embodiments, X⁶ is N. In some embodiments, X⁶ is selected from those depicted in Table A below. In some embodiments, X⁶ is selected from those depicted in Table A-2 below.

As defined generally above, X⁷ is CH or N. In some embodiments, X⁷ is N. In embodiments, X⁷ is CH. In some embodiments, X⁶ is selected from those depicted in Table A below. In some embodiments, X⁶ is selected from those depicted in Table A-2 below.

As defined generally above, R¹⁸, R¹⁹, R²⁰, and R²¹ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy.

In some embodiments, R¹⁸ is hydrogen. In some embodiments, R¹⁸ is an optionally substituted C₁₋₆ aliphatic group. In some embodiments, R¹⁸ is halogen. In some embodiments, R¹⁸ is —OR. In some embodiments, R¹⁸ is —CN. In some embodiments, R¹⁸ is —NR₂. In some embodiments, R¹⁸ is —C(═O)R. In some embodiments, R¹⁸ is —C(═O)OR. In some embodiments, R¹⁸ is —C(═O)NR₂. In some embodiments, R¹⁸ is —SO₂R. In some embodiments, R¹⁸ is —SO₂NR₂. In some embodiments, R¹⁸ is C₁₋₆haloalkyl. In some embodiments, R¹⁸ is C₁₋₆haloalkoxy. In some embodiments, R¹⁸ is —CD₃. In some embodiments, R¹⁸ is selected from those depicted in Table A below. In some embodiments, R¹⁸ is selected from those depicted in Table A-2 below.

In some embodiments, R¹⁹ is hydrogen. In some embodiments, R¹⁹ is an optionally substituted C₁₋₆ aliphatic group. In some embodiments, R¹⁹ is halogen. In some embodiments, R¹⁹ is —OR. In some embodiments, R¹⁹ is —CN. In some embodiments, R¹⁹ is —NR₂. In some embodiments, R¹⁹ is —C(═O)R. In some embodiments, R¹⁹ is —C(═O)OR. In some embodiments, R¹⁹ is —C(═O)NR₂. In some embodiments, R¹⁹ is —SO₂R. In some embodiments, R¹⁹ is —SO₂NR₂. In some embodiments, R¹⁹ is C₁₋₆haloalkyl. In some embodiments, R¹⁹ is C₁₋₆haloalkoxy. In some embodiments, R¹⁹ is —CD₃. In some embodiments, R¹⁹ is selected from those depicted in Table A below. In some embodiments, R¹⁹ is selected from those depicted in Table A-2 below.

In some embodiments, R²⁰ is hydrogen. In some embodiments, R²⁰ is an optionally substituted C₁₋₆ aliphatic group. In some embodiments, R²⁰ is halogen. In some embodiments, R²⁰ is —OR. In some embodiments, R²⁰ is —CN. In some embodiments, R²⁰ is —NR₂. In some embodiments, R²⁰ is —C(═O)R. In some embodiments, R²⁰ is —C(═O)OR. In some embodiments, R²⁰ is —C(═O)NR₂. In some embodiments, R²⁰ is —SO₂R. In some embodiments, R²⁰ is —SO₂NR₂. In some embodiments, R²⁰ is C₁₋₆haloalkyl. In some embodiments, R²⁰ is C₁₋₆haloalkoxy. In some embodiments, R²⁰ is —CD₃. In some embodiments, R²⁰ is selected from those depicted in Table A below. In some embodiments, R²⁰ is selected from those depicted in Table A-2 below.

In some embodiments, R²¹ is hydrogen. In some embodiments, R²¹ is an optionally substituted C₁₋₆ aliphatic group. In some embodiments, R²¹ is halogen. In some embodiments, R²¹ is —OR. In some embodiments, R²¹ is —CN. In some embodiments, R²¹ is —NR₂. In some embodiments, R²¹ is —C(═O)R. In some embodiments, R²¹ is —C(═O)OR. In some embodiments, R²¹ is —C(═O)NR₂. In some embodiments, R²¹ is —SO₂R. In some embodiments, R²¹ is —SO₂NR₂. In some embodiments, R²¹ is C₁₋₆haloalkyl. In some embodiments, R²¹ is C₁₋₆haloalkoxy. In some embodiments, R²¹ is —CD₃. In some embodiments, R²¹ is selected from those depicted in Table A below. In some embodiments, R²¹ is selected from those depicted in Table A-2 below.

As defined generally above, n is 0 or 1; provided that when X¹ is N and n is 0, X² is not NH or O. In some embodiments, n is 0. In some embodiments, n is 1. In some embodiments, X¹ is N, n is 0, and X² is not NH or 0.

As defined generally above, R¹ is H or C₁₋₃ alkyl. In some embodiments, R¹ is H or methyl. In some embodiments, R¹ is H. In some embodiments, R¹ is selected from those depicted in Table A below. In some embodiments, R¹ is selected from those depicted in Table A-2 below.

In some embodiments, the compound is a compound of Formula IIIa:

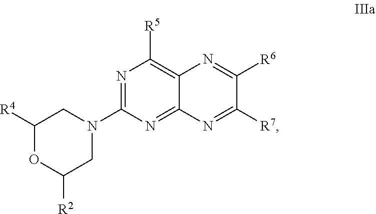

or a pharmaceutically acceptable salt thereof, wherein each variable is as defined above and described in embodiments herein both singly and in combination.

In some embodiments, the compound is a compound of Formula IIIb:

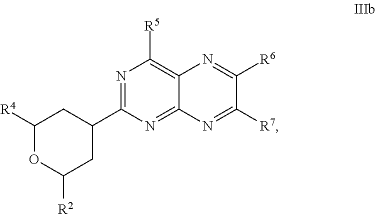

or a pharmaceutically acceptable salt thereof, wherein each variable is as defined above and described in embodiments herein both singly and in combination.

In some embodiments, the compound is a compound of Formula IIIc:

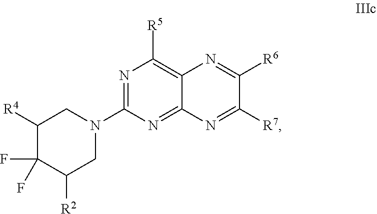

or a pharmaceutically acceptable salt thereof, wherein each variable is as defined above and described in embodiments herein both singly and in combination.

In some embodiments, the compound is a compound of Formula IIId:

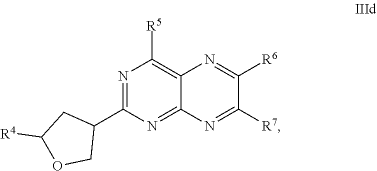

or a pharmaceutically acceptable salt thereof, wherein each variable is as defined above and described in embodiments herein both singly and in combination.

In some embodiments, the compound is a compound of Formula IIIe:

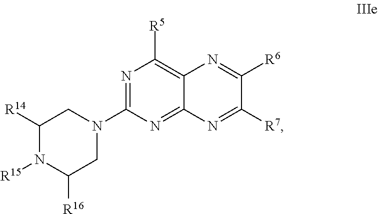

or a pharmaceutically acceptable salt thereof, wherein each variable is as defined above and described in embodiments herein both singly and in combination.

In some embodiments, the compound is a compound of Formula IIIf:

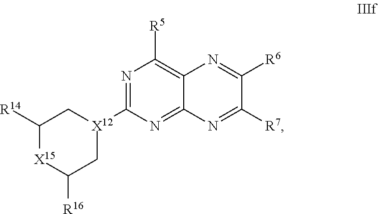

or a pharmaceutically acceptable salt thereof, wherein each variable is as defined above and described in embodiments herein both singly and in combination.

In some embodiments, the compound is a compound of Formula IVa:

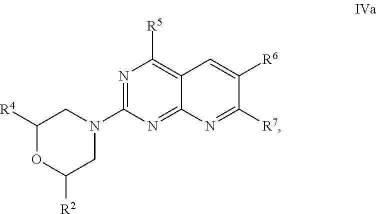

or a pharmaceutically acceptable salt thereof, wherein each variable is as defined above and described in embodiments herein both singly and in combination.

In some embodiments, the compound is a compound of Formula IVb:

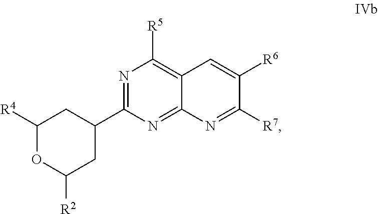

or a pharmaceutically acceptable salt thereof, wherein each variable is as defined above and described in embodiments herein both singly and in combination.

In some embodiments, the compound is a compound of Formula IVc:

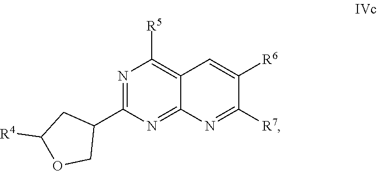

or a pharmaceutically acceptable salt thereof, wherein each variable is as defined above and described in embodiments herein both singly and in combination.

In some embodiments, the compound is a compound of Formula IVd:

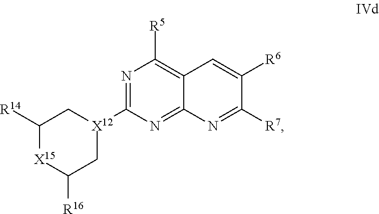

or a pharmaceutically acceptable salt thereof, wherein each variable is as defined above and described in embodiments herein both singly and in combination.

In some embodiments, the compound is a compound of Formula Va:

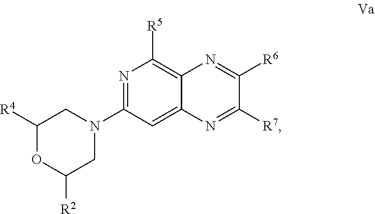

or a pharmaceutically acceptable salt thereof, wherein each variable is as defined above and described in embodiments herein both singly and in combination.

In some embodiments, the compound is a compound of Formula Vb:

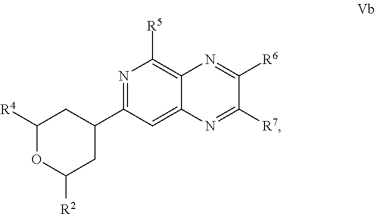

or a pharmaceutically acceptable salt thereof, wherein each variable is as defined above and described in embodiments herein both singly and in combination.

In some embodiments, the compound is a compound of Formula Vc:

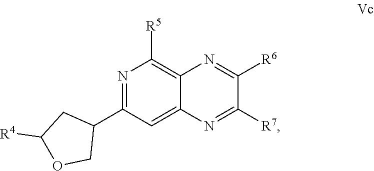

or a pharmaceutically acceptable salt thereof, wherein each variable is as defined above and described in embodiments herein both singly and in combination.

In some embodiments, the compound is a compound of Formula Vd:

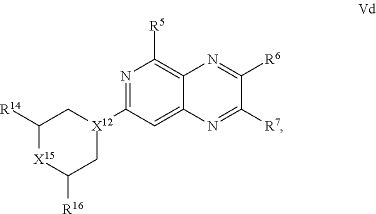

or a pharmaceutically acceptable salt thereof, wherein each variable is as defined above and described in embodiments herein both singly and in combination.

In some embodiments, the compound is a compound of Formula VIa:

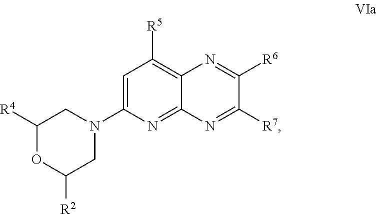

or a pharmaceutically acceptable salt thereof, wherein each variable is as defined above and described in embodiments herein both singly and in combination.

In some embodiments, the compound is a compound of Formula VIb:

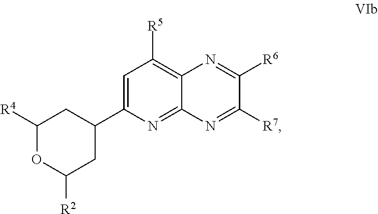

or a pharmaceutically acceptable salt thereof, wherein each variable is as defined above and described m embodiments herein both singly and in combination.

In some embodiments, the compound is a compound of Formula VIc:

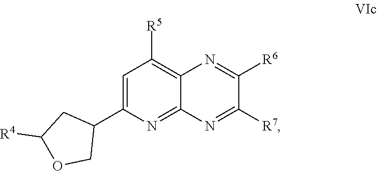

or a pharmaceutically acceptable salt thereof, wherein each variable is as defined above and described in embodiments herein both singly and in combination.

In some embodiments, the compound is a compound of Formula VId:

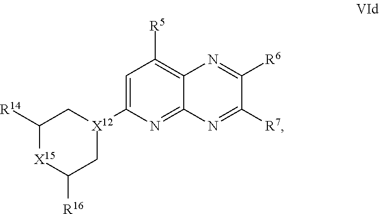

or a pharmaceutically acceptable salt thereof, wherein each variable is as defined above and described in embodiments herein both singly and in combination.

In some embodiments, the compound is a compound of Formula VIIa:

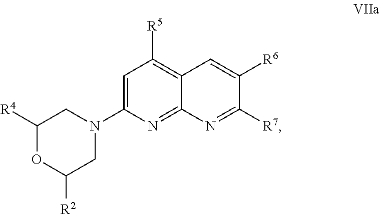

or a pharmaceutically acceptable salt thereof, wherein each variable is as defined above and described in embodiments herein both singly and in combination.

In some embodiments, the compound is a compound of Formula VIIb:

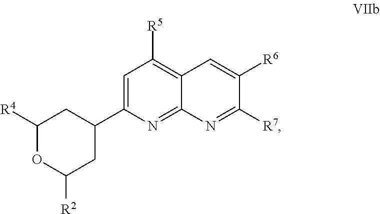

or a pharmaceutically acceptable salt thereof, wherein each variable is as defined above and described in embodiments herein both singly and in combination.

In some embodiments, the compound is a compound of Formula VIIc:

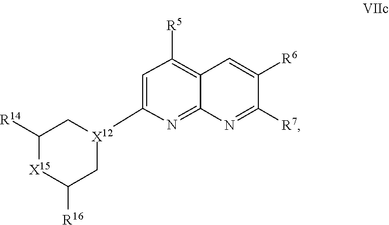

or a pharmaceutically acceptable salt thereof, wherein each variable is as defined above and described in embodiments herein both singly and in combination.

In some embodiments, the compound is a compound of Formula VIIIa:

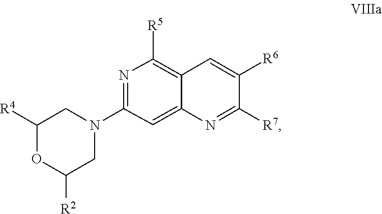

or a pharmaceutically acceptable salt thereof, wherein each variable is as defined above and described in embodiments herein both singly and in combination.

In some embodiments, the compound is a compound of Formula VIIIb:

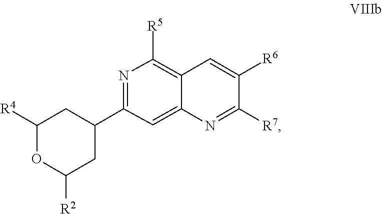

or a pharmaceutically acceptable salt thereof, wherein each variable is as defined above and described in embodiments herein both singly and in combination.

In some embodiments, the compound is a compound of Formula VIIIc:

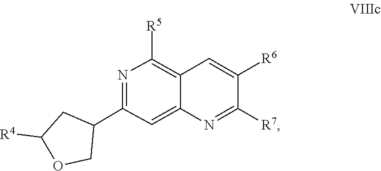

or a pharmaceutically acceptable salt thereof, wherein each variable is as defined above and described in embodiments herein both singly and in combination.

In some embodiments, the compound is a compound of Formula VIIId:

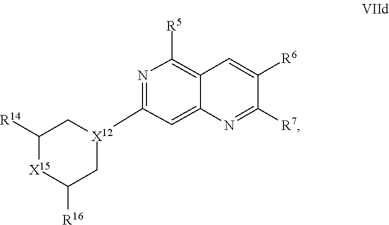

or a pharmaceutically acceptable salt thereof, wherein each variable is as defined above and described in embodiments herein both singly and in combination.

In some embodiments, the compound is a compound of Formula IXa:

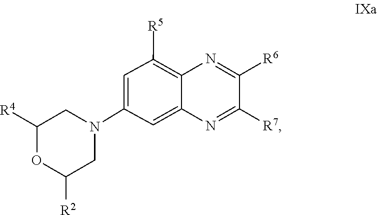

or a pharmaceutically acceptable salt thereof, wherein each variable is as defined above and described in embodiments herein both singly and in combination.

In some embodiments, the compound is a compound of Formula IXb:

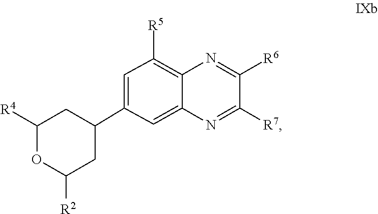

or a pharmaceutically acceptable salt thereof, wherein each variable is as defined above and described in embodiments herein both singly and in combination.

In some embodiments, the compound is a compound of Formula IXc:

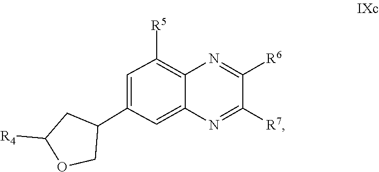

or a pharmaceutically acceptable salt thereof, wherein each variable is as defined above and described in embodiments herein both singly and in combination.

In some embodiments, the compound is a compound of Formula IXd:

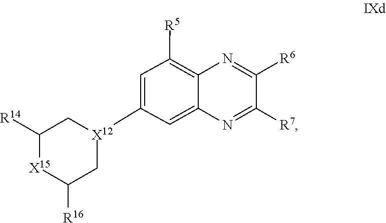

or a pharmaceutically acceptable salt thereof, wherein each variable is as defined above and described in embodiments herein both singly and in combination.

In some embodiments, the compound is a compound of Formula Xa:

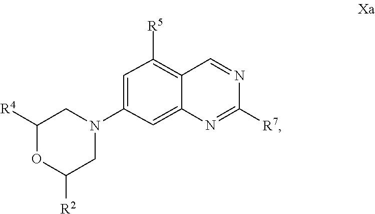

or a pharmaceutically acceptable salt thereof, wherein each variable is as defined above and described in embodiments herein both singly and in combination.

In some embodiments, the compound is a compound of Formula Xb:

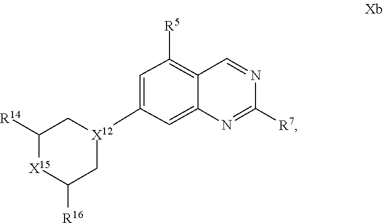

or a pharmaceutically acceptable salt thereof, wherein each variable is as defined above and described in embodiments herein both singly and in combination.

In some embodiments, the compound is a compound of Formula XIa:

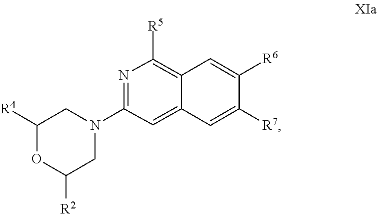

or a pharmaceutically acceptable salt thereof, wherein each variable is as defined above and described in embodiments herein both singly and in combination.

In some embodiments, the compound is a compound of Formula XIb:

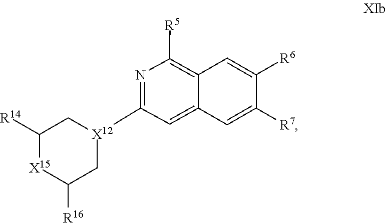

or a pharmaceutically acceptable salt thereof, wherein each variable is as defined above and described in embodiments herein both singly and in combination.

In some embodiments, the compound is a compound of Formula XIIa:

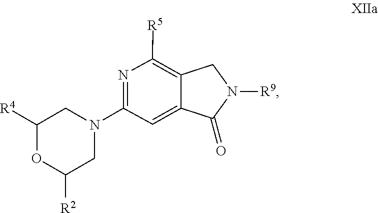

or a pharmaceutically acceptable salt thereof, wherein each variable is as defined above and described in embodiments herein both singly and in combination.

In some embodiments, the compound is a compound of Formula XIIb:

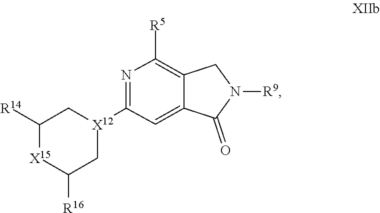

or a pharmaceutically acceptable salt thereof, wherein each variable is as defined above and described in embodiments herein both singly and in combination.

As defined generally above, R² is H or C₁₋₃ alkyl. In some embodiments, R² is H or methyl. In some embodiments, R² is H. In some embodiments, R² is methyl. In some embodiments, R² is selected from those depicted in Table A below.

As defined generally above, R³ is H or C₁₋₃ alkyl. In some embodiments, R³ is H or methyl. In some embodiments, R³ is H. In some embodiments, R³ is selected from those depicted in Table A below.

As defined generally above, R⁴ is C₁₋₆alkyl, C₁₋₆haloalkyl, diC₁₋₃alkylamino, —C(═O)O(C₁₋₆alkyl), C₃₋₆cycloalkyl, C₃₋₆heterocycloalkyl, phenyl, 5-membered heteroaryl, or 6-membered heteroaryl; wherein
(1) the C₃₋₆cycloalkyl or the C₃₋₆heterocycloalkyl is optionally substituted with C═O;
(2) the phenyl, 5-membered heteroaryl, or 6-membered heteroaryl group is optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein
the C₁₋₆alkyl and C₁₋₆haloalkyl of subsection (2) are optionally substituted with OH; and wherein
the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is C₁₋₆alkyl, C₃₋₆heterocycloalkyl, 5-membered heteroaryl, or 6-membered heteroaryl; wherein the 5-membered heteroaryl or 6-membered heteroaryl group is optionally substituted with 1 to 3 substituents independently selected from C₁₋₆alkyl, C₁₋₆alkoxy, C₃₋₆cycloalkyl, and C₃₋₆heterocycloalkyl. In some embodiments, R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl. In some embodiments, R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl and C₃₋₆cycloalkyl. In some embodiments, R⁴ is 5-membered heteroaryl optionally substituted with 1 to 3 substituents independently selected from C₁₋₆alkyl and C₃₋₆cycloalkyl. In some embodiments, R⁴ is 6-membered heteroaryl optionally substituted with 1 to 3 substituents independently selected from C₁₋₆alkyl and C₃₋₆cycloalkyl.

In some embodiments, R⁴ is 5-membered heteroaryl or 6-membered heteroaryl; wherein the 5-membered heteroaryl or 6-membered heteroaryl group is substituted with a C₃₋₆cycloalkyl; wherein the C₃₋₆cycloalkyl is optionally substituted with 1 to 3 substituents selected from halogen, C₁₋₃alkyl, and —C(═O)O(C₁₋₆alkyl). In some embodiments, R⁴ is 5-membered heteroaryl substituted with a C₃₋₆cycloalkyl; wherein the C₃₋₆cycloalkyl is optionally substituted with 1 to 3 substituents selected from halogen, C₁₋₃alkyl, and —C(═O)O(C₁₋₆alkyl). In some embodiments, R⁴ is 6-membered heteroaryl substituted with a C3. ₆cycloalkyl; wherein the C₃₋₆cycloalkyl is optionally substituted with 1 to 3 substituents selected from halogen, C₁₋₃alkyl, and —C(═O)O(C₁₋₆alkyl). In some embodiments, R⁴ is 5-membered heteroaryl or 6-membered heteroaryl; wherein the 5-membered heteroaryl or 6-membered heteroaryl group is substituted with a C₁₋₆haloalkyl. In some embodiments, R⁴ is 5-membered heteroaryl substituted with a C₁₋₆haloalkyl. In some embodiments, R⁴ is 6-membered heteroaryl substituted with a C₁₋₆haloalkyl. In some embodiments, R⁴ is 5-membered heteroaryl or 6-membered heteroaryl; wherein the 5-membered heteroaryl or 6-membered heteroaryl group is substituted with a C₁₋₆alkoxy. In some embodiments, R⁴ is 5-membered heteroaryl substituted with a C₁₋₆alkoxy. In some embodiments, R⁴ is 6-membered heteroaryl substituted with a C₁₋₆alkoxy.

In some embodiments, R⁴ is pyridinyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is pyrazolyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is pyrimidinyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is pyridazinyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is triazolyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is oxadiazolyl, optionally substituted with 1 to 3 substituents independently selected from halogen, C₁₋₆alkyl, C₁₋₆haloalkyl, C₁₋₆alkoxy, C₁₋₆haloalkoxy, —(C₁₋₃alkyl)O(C₁₋₃alkyl), —CN, C₂₋₄alkenyl, C₃₋₆cycloalkyl, and C₃₋₆heterocycloalkyl; wherein the C₁₋₆alkyl and C₁₋₆haloalkyl of subsection (2) are optionally substituted with OH; and wherein the C₃₋₆heterocycloalkyl of subsection (2) is optionally substituted with 1 to 3 substituents selected from halogen, C₁₋₃alkyl, and —C(═O)O(C₁₋₆alkyl).

In some embodiments, R⁴ is methyl, tetrahydrofuran-3-yl,

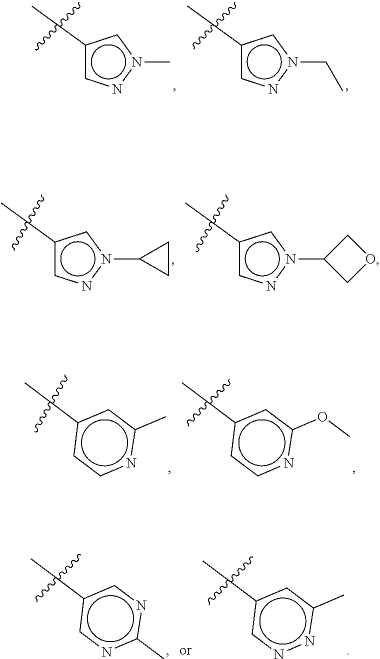

In some embodiments, R⁴ is methyl, tetrahydrofuran-3-yl,

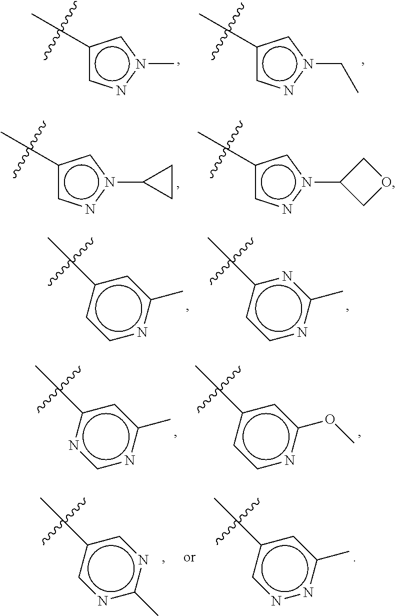

In some embodiments, R⁴ is methyl, tetrahydrofuran-3-yl,

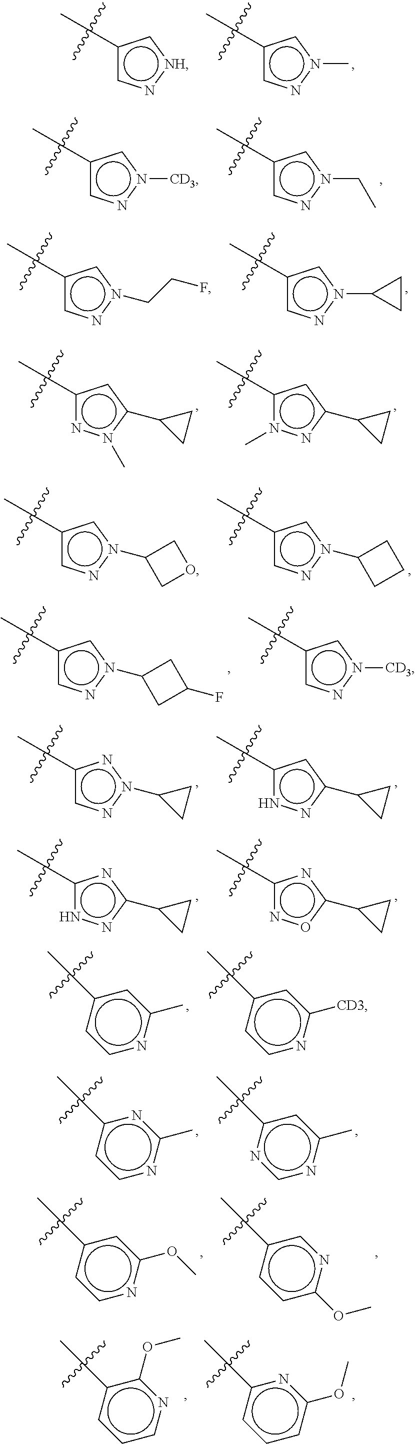

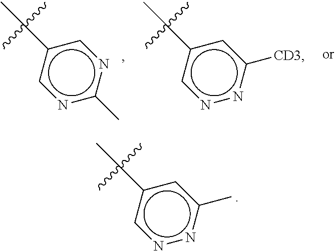

In some embodiments, R⁴ is

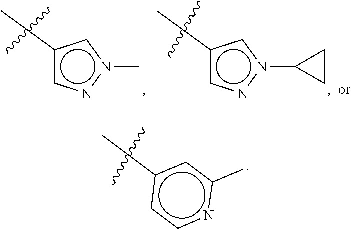

In some embodiments, R⁴ is

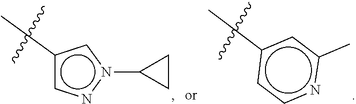

In some embodiments, R⁴ is

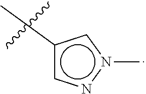

In some embodiments, R⁴ is

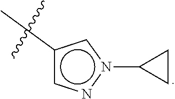

In some embodiments, R⁴ is

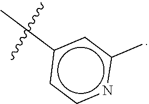

In some embodiments, R⁴ is

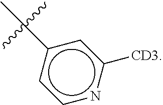

In some embodiments, R⁴ is a substituent selected from those shown below:

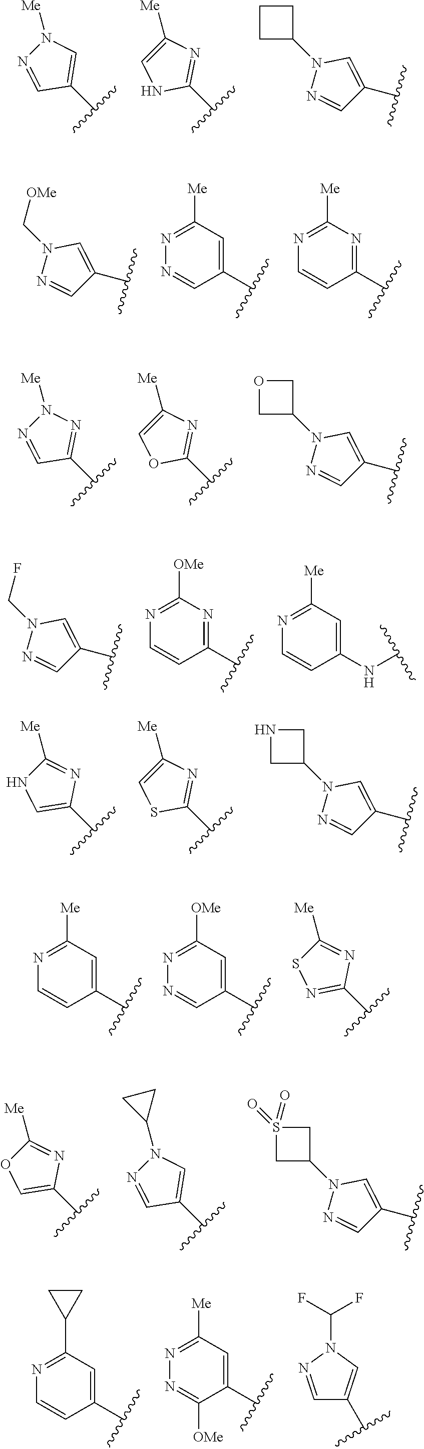

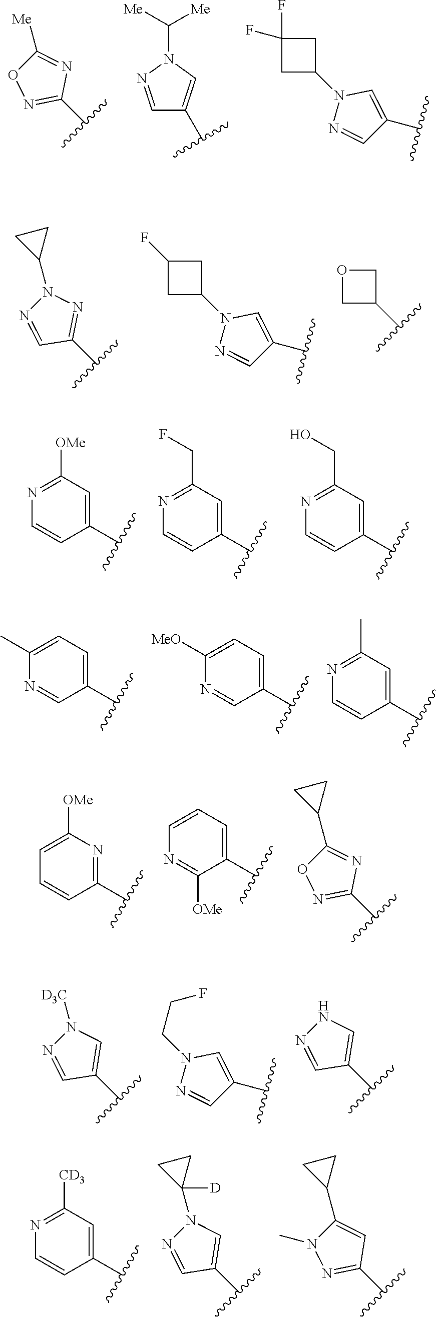

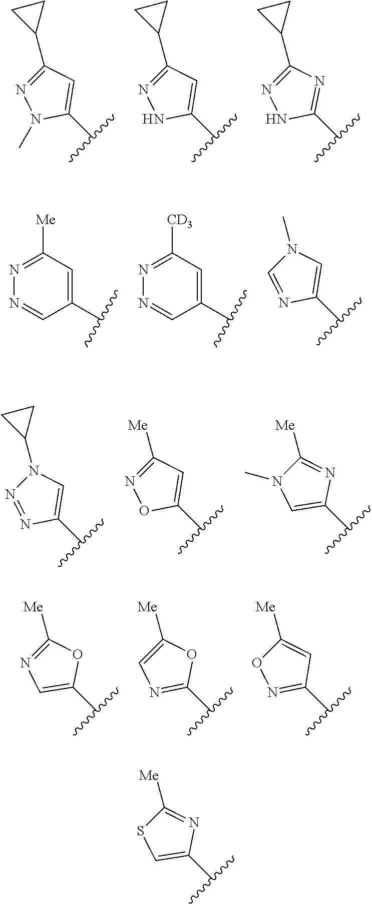

In some embodiments, R is substituted with C₁₋₃alky, comprising one or more deuteriums. In some embodiments, R⁴ is substituted with 1 to 3 substitutents selected from —CD₃, —CHD₂, and —CH₂D.

In some embodiments, R⁴ is selected from those depicted in Table A below.

As defined generally above, R⁵ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃-₆cycloalkyl), or a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R is an optionally substituted C₁₋₆ aliphatic group. In some embodiments, R⁵ is —OR. In some embodiments, R is —NR₂. In some embodiments, R is —C(═O)R. In some embodiments, R⁵ is —C(═O)OR. In some embodiments, R is —C(═O)NR₂. In some embodiments, R⁵ is —SO₂R. In some embodiments, R⁵ is —SO₂NR₂. In some embodiments, R is C₁₋₆haloalkyl. In some embodiments, R⁵ is an optionally substituted OCH₂—(C₃₋₆cycloalkyl). In some embodiments, R⁵ is an optionally substituted 3-8 membered saturated or partially unsaturated monocyclic carbocyclic ring. In some embodiments, R⁵ is an optionally substituted 5-12 membered saturated or partially unsaturated bridged carbocyclic ring. In some embodiments, R⁵ is an optionally substituted 7-12 membered saturated or partially unsaturated bicyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 3-8 membered saturated or partially unsaturated monocyclic heterocyclic ring (having 1-2 heteroatoms independently selected from nitrogen, oxygen, and sulfur). In some embodiments, R⁵ is an optionally substituted 6-12 membered saturated or partially unsaturated bridged heterocyclic ring (having 1-4 heteroatoms independently selected from nitrogen, oxygen, and sulfur). In some embodiments, R⁵ is an optionally substituted 7-12 membered saturated or partially unsaturated bicyclic heterocyclic ring (having 1-4 heteroatoms independently selected from nitrogen, oxygen, and sulfur). In some embodiments, R is an optionally substituted 5-6 membered monocyclic heteroaromatic ring (having 1-4 heteroatoms independently selected from nitrogen, oxygen, and sulfur). In some embodiments, R is an optionally substituted 8-10 membered bicyclic heteroaromatic ring (having 1-5 heteroatoms independently selected from nitrogen, oxygen, and sulfur).

In some embodiments, R is a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁵ is optionally substituted with 1-3 groups that are independently halogen; —(CH₂)₀₋₆R^{∘}; —(CH₂)₀₋₆—OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆—SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆—N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆—C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆SR^{∘},—(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆C(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆S(O)₂R^{∘}; —(CH₂)₀₋₆S(O)₂OR^{∘}; —(CH₂)₀₋₆OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined elsewhere herein and is independently hydrogen, C₁₋₆aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur). In some embodiments, R⁵ is optionally substituted with one or more —SF₅ groups.

In some embodiments, R⁵ is phenyl, optionally substituted with 1-3 substituents independently selected from halogen, C₁₋₆ aliphatic, —OR^{∘}, or C₁₋₆haloalkyl. In some embodiments, R⁵ is phenyl, optionally substituted with 1-3 halogen. In some embodiments, R⁵ is a 5-12 membered saturated or partially unsaturated bridged carbocyclic ring, optionally substituted with 1-3 substituents independently selected from halogen, C₁₋₆ aliphatic, —OR^{∘}, or C₁₋₆haloalkyl. In some embodiments, R⁵ is a C₅₋₈tricycloalkyl ring, optionally substituted with 1-3 substituents independently selected from halogen, C₁₋₆ aliphatic, —OR^{∘}, or C₁₋₆haloalkyl. In some embodiments, R⁵ is 5-6 membered monocyclic heteroaromatic ring (having 1-4 heteroatoms independently selected from nitrogen, oxygen, and sulfur), optionally substituted with 1-3 substituents independently selected from halogen, C₁₋₆ aliphatic, —OR^{∘}, or C₁₋₆haloalkyl. In some embodiments, R⁵ is 5-6 membered monocyclic heteroaromatic ring (having 1-4 heteroatoms independently selected from nitrogen, oxygen, and sulfur), optionally substituted with 1-3 halogen.

As defined generally in Formula I above, R⁵ is C₁₋₆alkyl, C₁₋₆haloalkyl, C₃₋₆cycloalkyl, C₅₋₈spiroalkyl, C5-stricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl), wherein the C₁₋₆alkyl, C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, C₁₋₃alkoxy, and C₁₋₃haloalkoxy.

In some embodiments, R⁵ is C₁ₗhaloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl), wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy.

In some embodiments, R⁵ is C₁₋₆haloalkyl. In some embodiments, R⁵ is C₃₋₆cycloalkyl optionally substituted with 1 to 4 substituents independently selected from halogen, C₁₋₃alkyl, and C₁₋₃haloalkyl. In some embodiments, R⁵ is C₅₋₈ spiroalkyl optionally substituted with 1 to 4 substituents independently selected from halogen, C₁₋₃alkyl, and C₁₋₃haloalkyl. In some embodiments, R⁵ is C₅₋₈tricycloalkyl optionally substituted with 1 to 4 substituents independently selected from halogen, C₁₋₃alkyl, and C₁₋₃haloalkyl. In some embodiments, R⁵ is cyclopent-1-en-1-yl optionally substituted with 1 to 4 substituents independently selected from halogen, C₁₋₃alkyl, and C₁₋₃haloalkyl. In some embodiments, R⁵ is cyclohex-1-en-1-yl optionally substituted with 1 to 4 substituents independently selected from halogen, C₁₋₃alkyl, and C₁₋₃haloalkyl. In some embodiments, R⁵ is phenyl optionally substituted with 1 to 4 substituents independently selected from halogen, C₁₋₃alkyl, and C₁₋₃haloalkyl. In some embodiments, R⁵ is 6-membered heteroaryl optionally substituted with 1 to 4 substituents independently selected from halogen, C₁₋₃alkyl, and C₁₋₃haloalkyl. In some embodiments, R⁵ is aziridine-1-yl substituted with 1 to 4 substituents independently selected from halogen, C₁₋₃alkyl, C₁₋₃haloalkyl, and C₁₋₃alkoxy. In some embodiments, R⁵ is pyrrolidine-1-yl substituted with 1 to 4 substituents independently selected from halogen, C₁₋₃alkyl, C₁₋₃haloalkyl, and C₁₋₃alkoxy. In some embodiments, R⁵ is azabicyclo[3.1.0]hexan-3-yl substituted with 1 to 4 substituents independently selected from halogen, C₁₋₃alkyl, C₁₋₃haloalkyl, and C₁₋₃alkoxy. In some embodiments, R⁵ is piperidine-1-yl substituted with 1 to 4 substituents independently selected from halogen, C₁₋₃alkyl, C₁₋₃haloalkyl, and C₁₋₃alkoxy. In some embodiments, R⁵ is —OCH₂—(C₃₋₆cycloalkyl) substituted with 1 to 4 substituents independently selected from halogen, C₁₋₃alkyl, C₁₋₃haloalkyl, and C₁₋₃alkoxy.

In some embodiments, R⁵ is —CH₂CH₂CF₃, optionally substituted C₃₋₆cycloalkyl, optionally substituted spiro[3.3]heptanyl, optionally substituted spiro[5.2]octanyl, optionally substituted

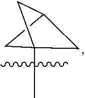 [SMILES: *C12CC(C1)C2]

optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted pipenidine-1-yl, or optionally substituted —OCH₂—(C₃₋₄cycloalkyl). In some embodiments, R⁵ is —CH₂CH₂CF₃. In some embodiments, R⁵ is optionally substituted C₃₋₆cycloalkyl. In some embodiments, R⁵ is optionally substituted spiro[3.3]heptanyl. In some embodiments, R⁵ is optionally substituted spiro[5.2]octanyl. In some embodiments, R⁵ is optionally substituted

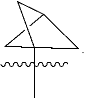 [SMILES: *C12CC(C1)C2]

In some embodiments, R⁵ is optionally substituted cyclopent-1-en-1-yl. In some embodiments, R⁵ is optionally substituted cyclohex-1-en-1-yl. In some embodiments, R⁵ is optionally substituted phenyl. In some embodiments, R⁵ is optionally substituted pyridinyl. In some embodiments, R⁵ is optionally substituted aziridine-1-yl. In some embodiments, R⁵ is optionally substituted pyrrolidine-1-yl. In some embodiments, R⁵ is optionally substituted azabicyclo[3.1.0]hexan-3-yl. In some embodiments, WI is optionally substituted piperidine-1-yl. In some embodiments, R⁵ is optionally substituted —OCH₂—(C₃₋₄cycloalkyl).

In some embodiments, R⁵ is a substituent selected from those shown below:

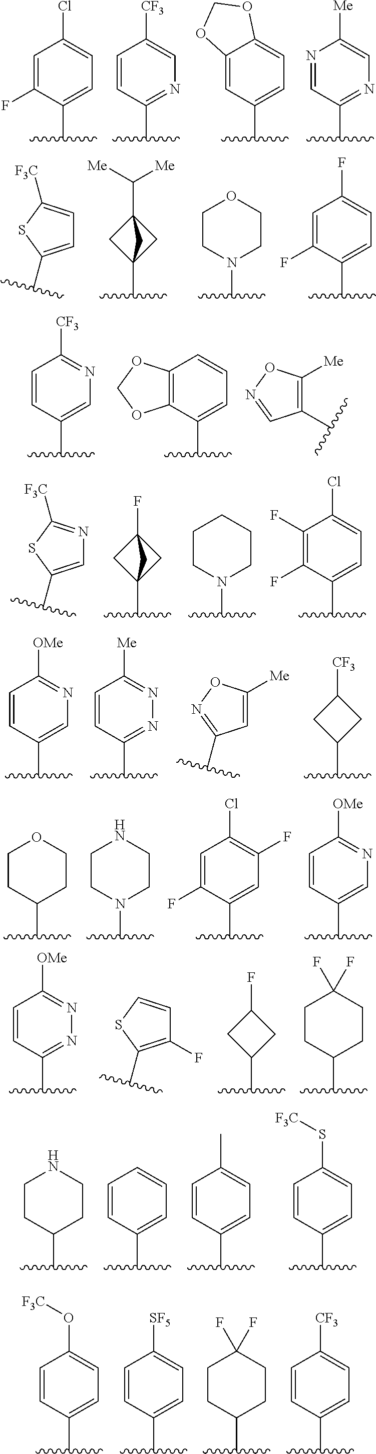

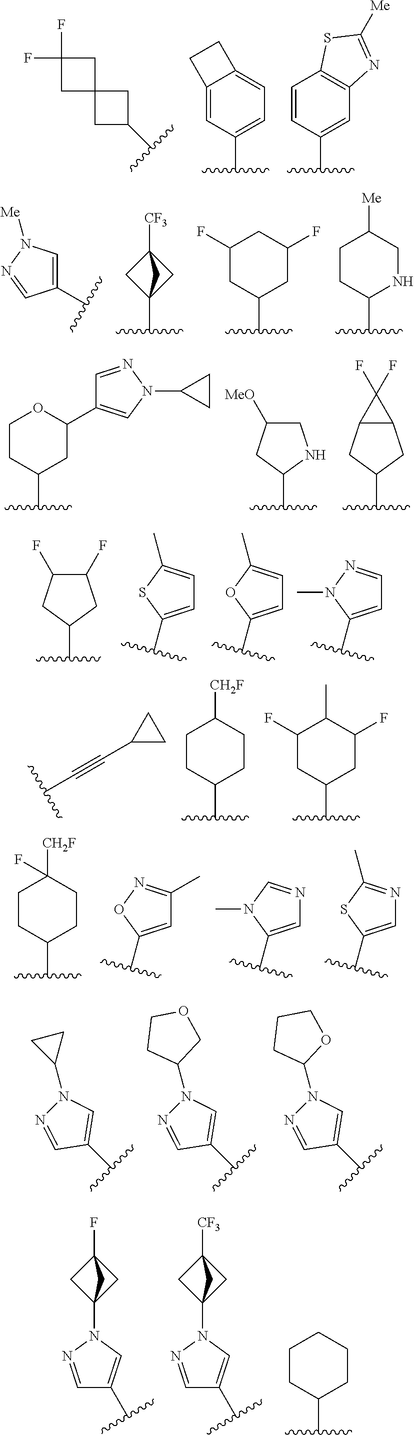

In some embodiments, R⁵ is —CH₂CH₂CF₃,

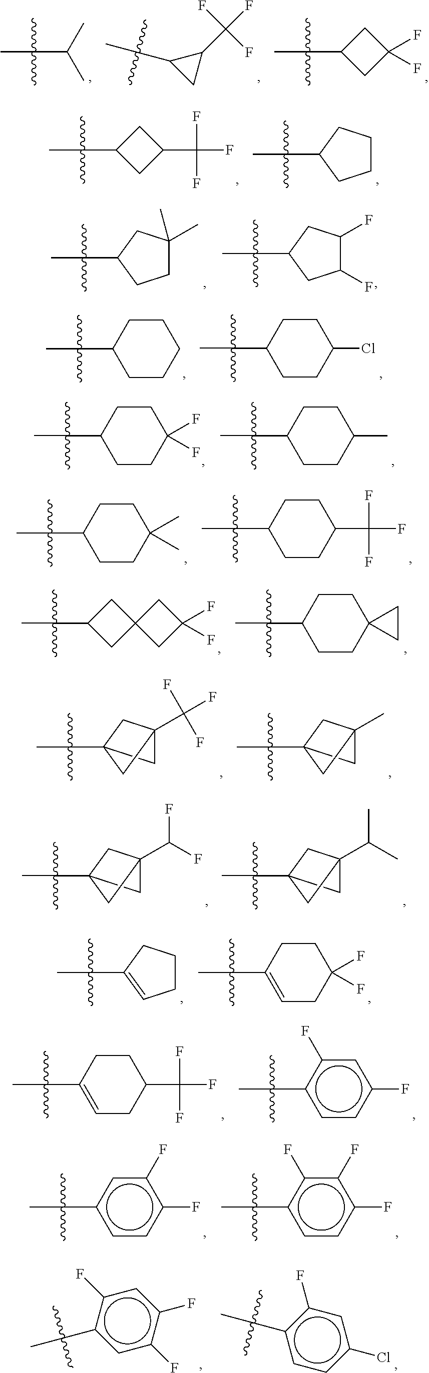

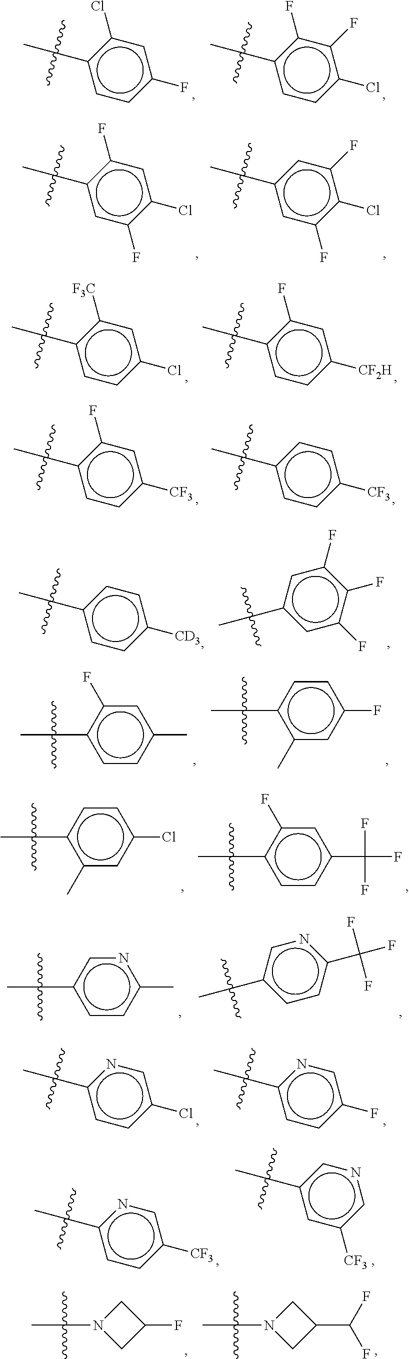

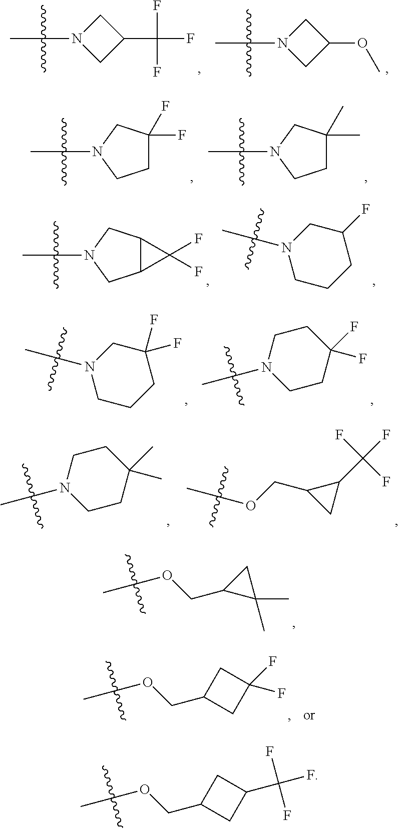

In some embodiments, R⁵ is

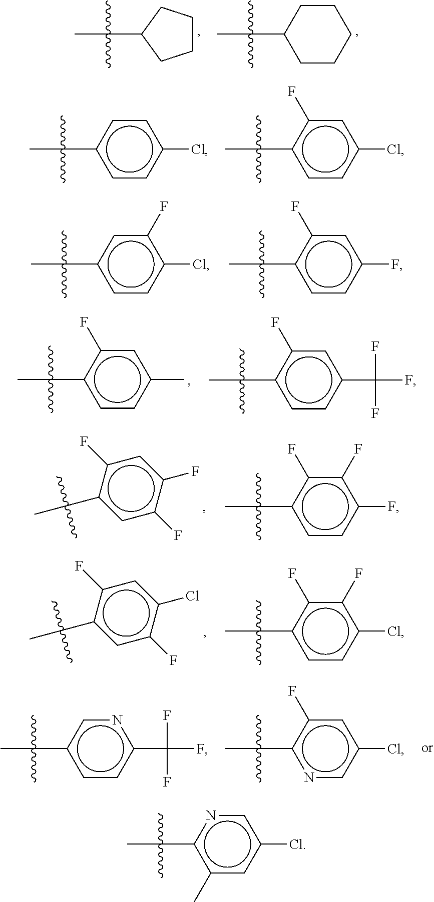

In some embodiments, R⁵ is —CH₂CH₂CF₃,

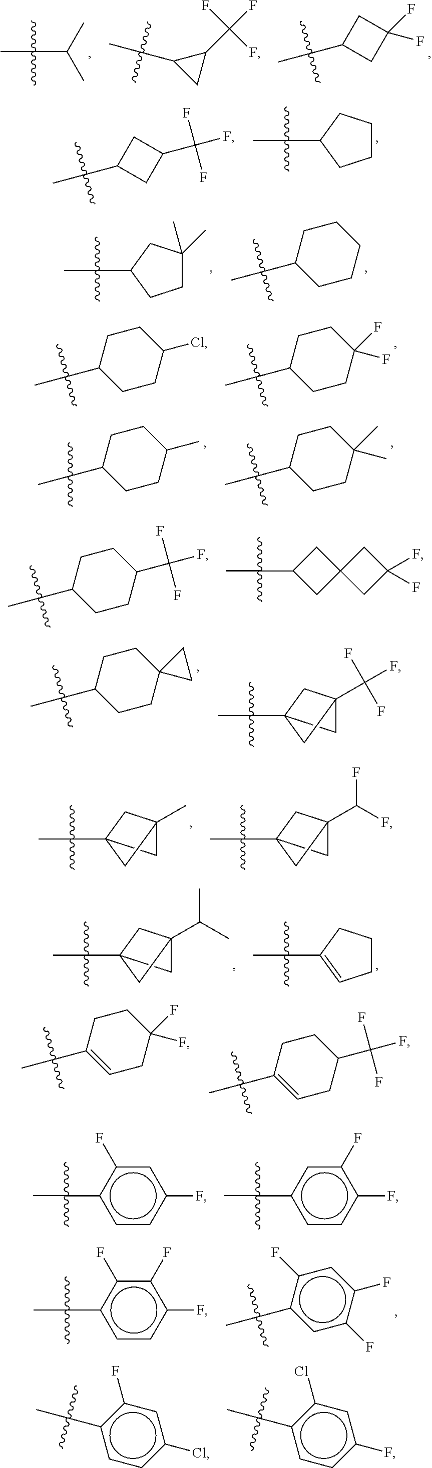

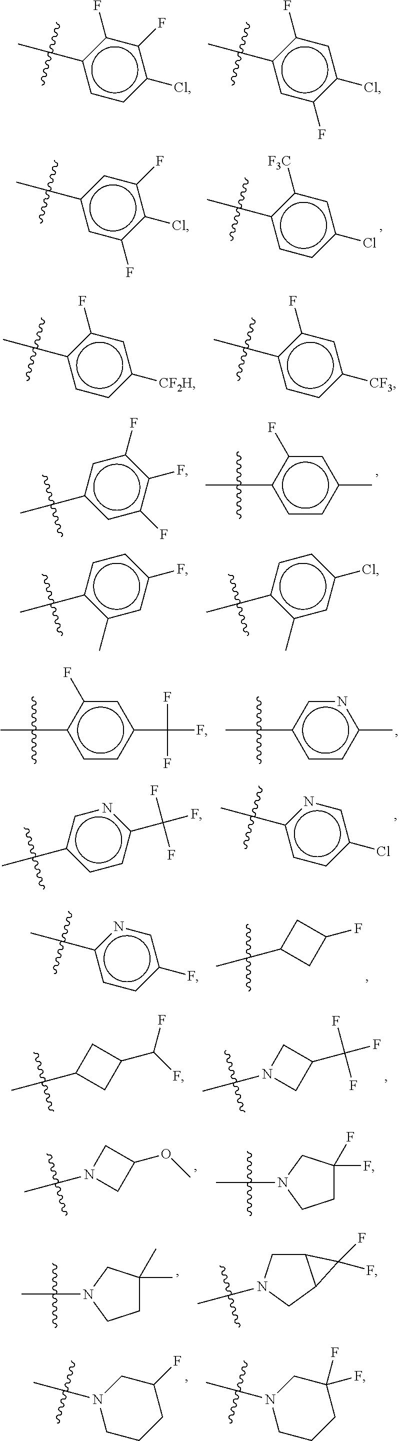

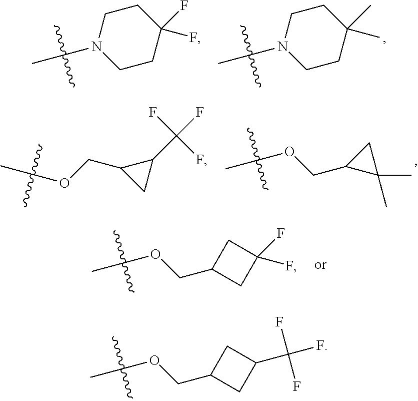

In some embodiments, R⁵ is

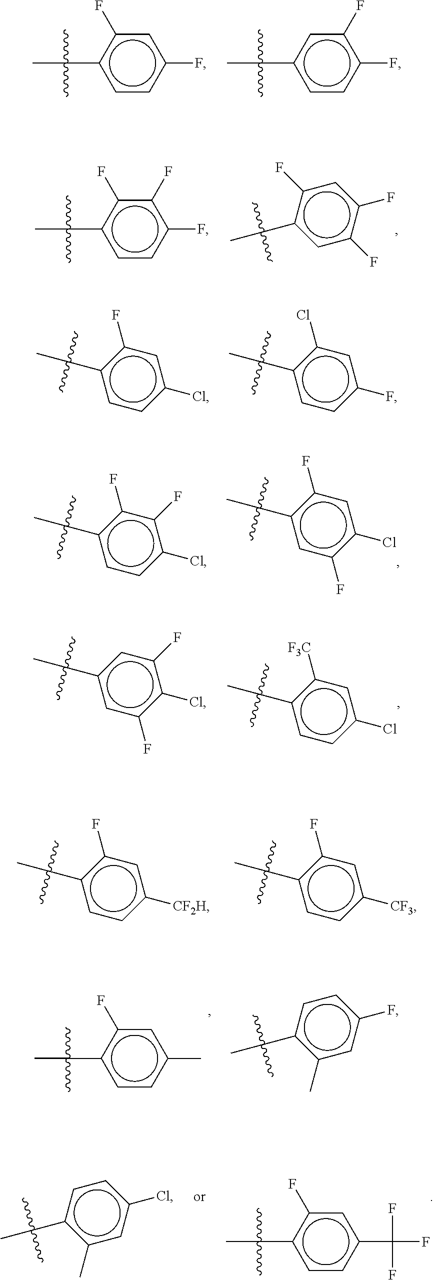

In some embodiments, R⁵ is

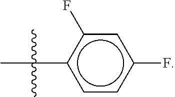

In some embodiments, R⁵ is

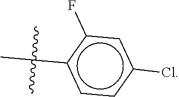

In some embodiments, R⁵ is

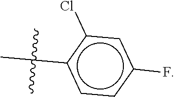

In some embodiments, R⁵ is

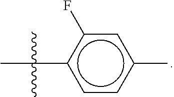

In some embodiments, R⁵ is optionally substituted C₃₋₆ cycloalkyl, optionally substituted spiro[3.3]heptanyl, optionally substituted spiro[5.2]octanyl, or optionally substituted

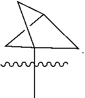 [SMILES: *C12CC(C1)C2]

In some embodiments, R⁵ is

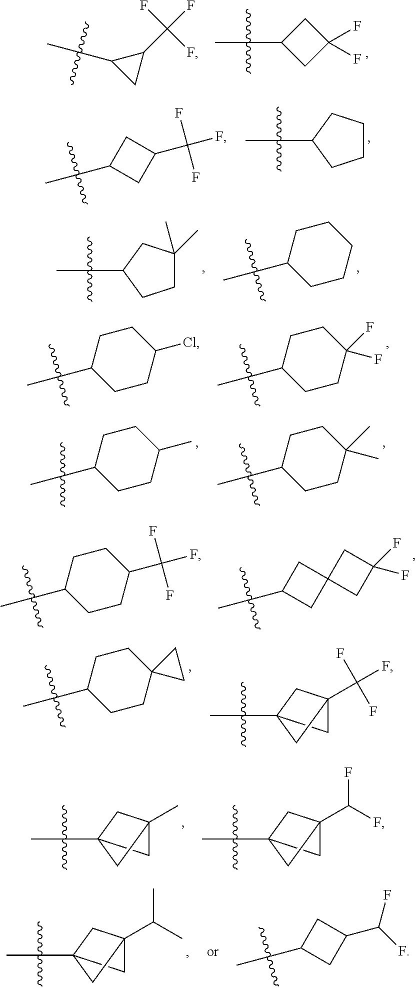

In some embodiments, R⁵ is

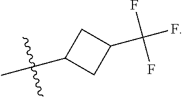 [SMILES: *C1CC(C(F)(F)F)C1]

In some embodiments, R⁵ is

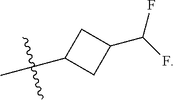 [SMILES: *C1CC(C(F)F)C1]

In some embodiments, R⁵ is

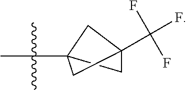 [SMILES: *C12CC(C(F)(F)F)(C1)C2]

In some embodiments, R⁵ is optionally substituted cyclopent-1-en-1-yl, or optionally substituted cyclohex-1-en-1-yl. In some embodiments, R⁵ is

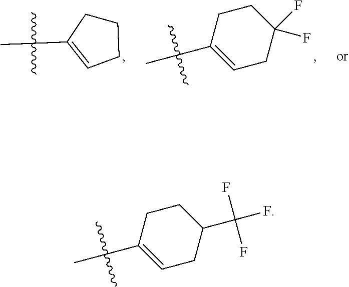 [SMILES: *C1=CCCC1 ; *C1=CCC(F)(F)CC1 ; *C1=CCC(C(F)(F)F)CC1]

In some embodiments, R⁵ is optionally substituted pyridinyl. In some embodiments, R⁵ is

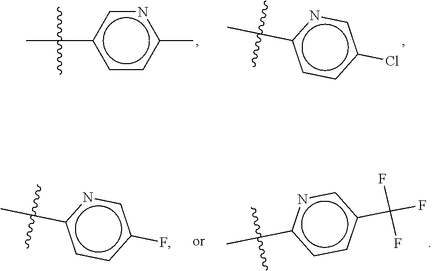

In some embodiments, R⁵ is substituted aziridine-1-yl, substituted pyrrolidine-1-yl, substituted azabicyclo[3.1.0]hexan-3-yl, or substituted piperidine-1-yl. In some embodiments, R⁵ is

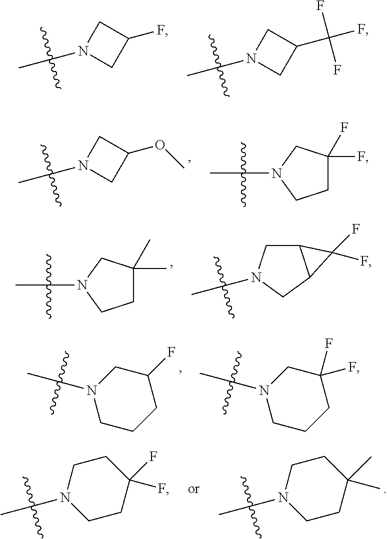

In some embodiments, R⁵ is

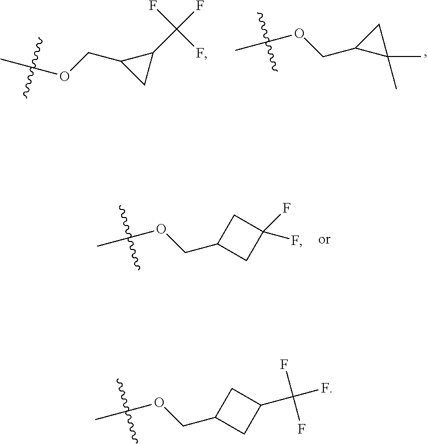

In some embodiments, R⁵ is selected from those depicted in Table A below.

As defined generally above, R⁶ and R⁷ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or R⁶ and R⁷ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁶ is an optionally substituted C₁₋₆ aliphatic group. In some embodiments, R⁶ is halogen. In some embodiments, R⁶ is —OR. In some embodiments, R⁶ is —NR₂. In some embodiments, R⁶ is —C(═O)R. In some embodiments, R⁶ is —C(═O)OR. In some embodiments, R⁶ is —C(═O)NR₂. In some embodiments, R⁶ is —SO₂R. In some embodiments, R⁶ is —SO₂NR₂. In some embodiments, R⁶ is C₁₋₆haloalkyl. In some embodiments, R⁶ is C₁₋₆haloalkoxy. In some embodiments, R⁶ is an optionally substituted 3-8 membered saturated or partially unsaturated monocyclic carbocyclic ring. In some embodiments, R⁶ is an optionally substituted 6-12 membered saturated or partially unsaturated bridged carbocyclic ring. In some embodiments, R⁶ is an optionally substituted 7-12 membered saturated or partially unsaturated bicyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 3-8 membered saturated or partially unsaturated monocyclic heterocyclic ring (having 1-2 heteroatoms independently selected from nitrogen, oxygen, and sulfur). In some embodiments, R⁶ is an optionally substituted 6-12 membered saturated or partially unsaturated bridged heterocyclic ring (having 1-4 heteroatoms independently selected from nitrogen, oxygen, and sulfur). In some embodiments, R⁶ is an optionally substituted 7-12 membered saturated or partially unsaturated bicyclic heterocyclic ring (having 1-4 heteroatoms independently selected from nitrogen, oxygen, and sulfur). In some embodiments, R⁶ is an optionally substituted 5-6 membered monocyclic heteroaromatic ring (having 1-4 heteroatoms independently selected from nitrogen, oxygen, and sulfur). In some embodiments, R⁶ is an optionally substituted 8-10 membered bicyclic heteroaromatic ring (having 1-5 heteroatoms independently selected from nitrogen, oxygen, and sulfur).

In some embodiments, R⁷ is an optionally substituted C₁₋₆ aliphatic group. In some embodiments, R⁷ is halogen. In some embodiments, R⁷ is —OR. In some embodiments, R⁷ is —NR₂. In some embodiments, R⁷ is —C(═O)R. In some embodiments, R⁷ is —C(═O)OR. In some embodiments, R⁷ is —C(═O)NR₂. In some embodiments, R⁷ is —SO₂R. In some embodiments, R⁷ is —SO₂NR₂. In some embodiments, R⁷ is C₁₋₆haloalkyl. In some embodiments, R⁷ is C₁₋₆haloalkoxy. In some embodiments, R⁷ is an optionally substituted 3-8 membered saturated or partially unsaturated monocyclic carbocyclic ring. In some embodiments, R⁷ is an optionally substituted 6-12 membered saturated or partially unsaturated bridged carbocyclic ring. In some embodiments, R⁷ is an optionally substituted 7-12 membered saturated or partially unsaturated bicyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 3-8 membered saturated or partially unsaturated monocyclic heterocyclic ring (having 1-2 heteroatoms independently selected from nitrogen, oxygen, and sulfur). In some embodiments, R⁷ is an optionally substituted 6-12 membered saturated or partially unsaturated bridged heterocyclic ring (having 1-4 heteroatoms independently selected from nitrogen, oxygen, and sulfur). In some embodiments, R⁷ is an optionally substituted 7-12 membered saturated or partially unsaturated bicyclic heterocyclic ring (having 1-4 heteroatoms independently selected from nitrogen, oxygen, and sulfur). In some embodiments, R⁷ is an optionally substituted 5-6 membered monocyclic heteroaromatic ring (having 1-4 heteroatoms independently selected from nitrogen, oxygen, and sulfur). In some embodiments, R⁷ is an optionally substituted 8-10 membered bicyclic heteroaromatic ring (having 1-5 heteroatoms independently selected from nitrogen, oxygen, and sulfur).

In some embodiments, R⁶ is hydrogen. In some embodiments, R⁶ is methyl. In some embodiments, R⁶ is Cl. In some embodiments, R⁶ is a C₁₋₃ haloalkyl. In some embodiments, R⁶ is 3-8 membered saturated monocyclic heterocyclic ring (having 1-2 heteroatoms independently selected from nitrogen, oxygen, and sulfur). In some embodiments, R⁶ is an azetidinyl group. In some embodiments, R⁶ is optionally substituted ethyl. In some embodiments, R⁶ is methoxy. In some embodiments, R⁶ is —CH₂F. In some embodiments, R⁶ is —OCH₂F. In some embodiments, R⁶ is —CD₃.

In some embodiments, R⁷ is hydrogen. In some embodiments, R⁷ is methyl. In some embodiments, R⁷ is Cl. In some embodiments, R⁷ is —CD₃.

In some embodiments, R⁶ and R⁷ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁶ and R⁷ are taken together with their intervening atoms to form an optionally substituted 3-8 membered saturated or partially unsaturated monocyclic carbocyclic ring. In some embodiments, R⁶ and R⁷ are taken together with their intervening atoms to form an optionally substituted 6-12 membered saturated or partially unsaturated bridged carbocyclic ring. In some embodiments, R⁶ and R⁷ are taken together with their intervening atoms to form an optionally substituted 7-12 membered saturated or partially unsaturated bicyclic carbocyclic ring. In some embodiments, R and R⁷ are taken together with their intervening atoms to form an optionally substituted phenyl. In some embodiments, R⁶ and R⁷ are taken together with their intervening atoms to form an optionally substituted 8-10 membered bicyclic aromatic carbocyclic ring. In some embodiments, R⁶ and R⁷ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R⁶ and R⁷ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R⁶ and R⁷ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R⁶ and R⁷ are taken together with their intervening atoms to form an optionally substituted 5-6 membered monocyclic heteroaromatic ring (having 1-4 heteroatoms independently selected from nitrogen, oxygen, and sulfur). In some embodiments, R⁶ and R⁷ are taken together with their intervening atoms to form an optionally substituted 8-10 membered bicyclic heteroaromatic ring (having 1-5 heteroatoms independently selected from nitrogen, oxygen, and sulfur).

As defined generally above in Formula I, R⁶ is H, halogen, or C₁₋₃alkyl. In some embodiments, R⁶ is H, chlorine, or methyl. In some embodiments, R is H or methyl. In some embodiments, R⁶ is H. In some embodiments, R⁶ is methyl. In some embodiments, R⁶ is selected from those depicted in Table A below.

As defined generally above in Formula I, R⁷ is H, halogen, or C₁₋₃alkyl. In some embodiments, R⁷ is H, methyl, or ethyl. In some embodiments, R⁷ is H. In some embodiments, R⁵ is methyl. In some embodiments, R⁷ is ethyl. In some embodiments, R⁷ is selected from those depicted in Table A below.

In some embodiments, R⁶ is H or methyl and R⁷ is H or methyl. In some embodiments, R⁶ is H or methyl and R⁷ is methyl. In some embodiments, R⁶ is H and R⁷ is methyl. In some embodiments, R⁶ is methyl and R⁷ is methyl. In some embodiments, R⁶ is Cl and R⁷ is methyl. In some embodiments, R⁶ is H and R⁷ is ethyl.

As defined generally above, R⁹ is H or C₁₋₅ alkyl. In some embodiments, R⁹ is H, methyl, ethyl, or iso-propyl. In some embodiments, R⁹ is methyl, ethyl, or iso-propyl. In some embodiments, R⁹ is methyl. In some embodiments, R⁹ is ethyl. In some embodiments, R⁹ is iso-propyl. In some embodiments, R⁹ is selected from those depicted in Table A below.

In some embodiments, Ring B is

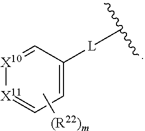

As defined above, L is a bond or an optionally substituted straight chain or branched C₁₋₆ alkylene. In some embodiments, L is a bond. In some embodiments, L is an optionally substituted straight chain or branched C₁₋₆ alkylene. In some embodiments, L is an optionally substituted ethylene. In some embodiments, L is an optionally substituted methylene.

As defined generally above, X¹⁰ is CH, N or CR¹⁰. In some embodiments, X¹⁰ is CH. In some embodiments, X¹⁰ is N. In some embodiments, X¹⁰ is CR¹⁰.

As defined generally above, X¹⁰ is CH, N or CR¹⁰. In some embodiments, X¹⁰ is CH. In some embodiments, X¹⁰ is N. In some embodiments, X¹⁰ is CR¹⁰.

In some embodiments, X¹⁰ is N and X¹¹ is CH. In some embodiments, X¹⁰ is N and X¹⁰ is CR¹⁰. In some embodiments, X¹⁰ is CH and X¹⁰ is N. In some embodiments, X¹⁰ is CR¹⁰ and X¹⁰ is N. In some embodiments, X¹⁰ is CH and X¹⁰ is CH. In some embodiments, X¹⁰ is CH and X¹⁰ is CR¹⁰. In some embodiments, X¹⁰ is CR¹⁰ and X¹⁰ is CH.

As defined generally above, R²² is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R²² is hydrogen. In some embodiments, R²² is an optionally substituted C₁₋₆ aliphatic group. In some embodiments, R²² is halogen. In some embodiments, R²² is —OR. In some embodiments, R²² is —CN. In some embodiments, R²² is —NR₂. In some embodiments, R²² is —C(═O)R. In some embodiments, R²² is —C(═O)OR. In some embodiments, R²² is —C(═O)NR₂. In some embodiments, R²² is —SO₂R. In some embodiments, R²² is —SO₂NR₂. In some embodiments, R²² is C₁₋₆haloalkyl. In some embodiments, R²² is C₁₋₆haloalkoxy. In some embodiments, R²² is —CD₃. In some embodiments, R²² is selected from those depicted in Table A below.

As defined generally above, m is 0, 1 or 2. In some embodiments, m is 0. In some embodiments, m is 1. In some embodiments, m is 2.

In some embodiments, Ring B is

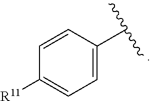

In some embodiments, Ring B is

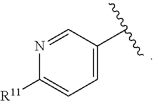

In some embodiments, Ring B is

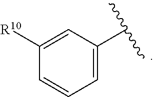

In some embodiments, Ring B is

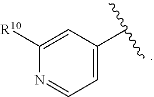

In some embodiments, Ring B is

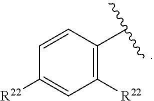

In some embodiments, Ring B is selected from those depicted in Table A below.

As defined generally above, R¹⁰ and R¹¹ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or R¹⁰ and R¹¹ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹⁰ is an optionally substituted C₁₋₆ aliphatic group. In some embodiments, R¹⁰ is —OR. In some embodiments, R¹⁰ is —NR₂. In some embodiments, R¹⁰ is —C(═O)R. In some embodiments, R¹⁰ is —C(═O)OR. In some embodiments, R¹⁰ is —C(═O)NR₂. In some embodiments, R⁵ is —SO₂R. In some embodiments, R¹⁰ is —SO₂NR₂. In some embodiments, R¹⁰ is halogen. In some embodiments, R¹⁰ is C₁₋₆haloalkyl. In some embodiments, R¹⁰ is C₁₋₆haloalkoxy. In some embodiments, R¹⁰ is an optionally substituted 3-8 membered saturated or partially unsaturated monocyclic carbocyclic ring. In some embodiments, R¹⁰ is an optionally substituted 6-12 membered saturated or partially unsaturated bridged carbocyclic ring. In some embodiments, R¹⁰ is an optionally substituted 7-12 membered saturated or partially unsaturated bicyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 3-8 membered saturated or partially unsaturated monocyclic heterocyclic ring (having 1-2 heteroatoms independently selected from nitrogen, oxygen, and sulfur). In some embodiments, R¹⁰ is an optionally substituted 6-12 membered saturated or partially unsaturated bridged heterocyclic ring (having 1-4 heteroatoms independently selected from nitrogen, oxygen, and sulfur). In some embodiments, R¹⁰ is an optionally substituted 7-12 membered saturated or partially unsaturated bicyclic heterocyclic ring (having 1-4 heteroatoms independently selected from nitrogen, oxygen, and sulfur). In some embodiments, R¹⁰ is an optionally substituted 5-6 membered monocyclic heteroaromatic ring (having 1-4 heteroatoms independently selected from nitrogen, oxygen, and sulfur). In some embodiments, R¹⁰ is an optionally substituted 8-10 membered bicyclic heteroaromatic ring (having 1-5 heteroatoms independently selected from nitrogen, oxygen, and sulfur).

In some embodiments, R¹⁰ is —OCF₃. In some embodiments, R¹⁰ is cyclopropyl. In some embodiments, R¹⁰ is cyclobutyl. In some embodiments, R¹⁰ is optionally substituted pyrazolyl. In some embodiments, R¹⁰ is optionally substituted pyridinyl. In some embodiments, R¹⁰ is optionally substituted pyrimidinyl. In some embodiments, R¹⁰ is optionally substituted pyridazinyl. In some embodiments, R¹⁰ is optionally substituted imidazolyl. In some embodiments, R¹⁰ is optionally substituted triazolyl. In some embodiments, R¹⁰ is optionally substituted oxazolyl. In some embodiments, R¹⁰ is optionally substituted thiazolyl. In some embodiments, R¹⁰ is optionally substituted oxadiazolyl. In some embodiments, R¹⁰ is optionally substituted thiadiazolyl. In some embodiments, R¹⁰ is optionally substituted oxetanyl. In some embodiments, R¹⁰ is optionally substituted azetidinyl. In some embodiments, R¹⁰ is optionally substituted piperidinyl. In some embodiments, R¹⁰ is optionally substituted piperazinyl. In some embodiments, R¹⁰ is selected from those depicted in Table A below.

In some embodiments, R¹¹ is an optionally substituted C₁₋₆ aliphatic group. In some embodiments, R¹¹ is —OR. In some embodiments, R¹¹ is —NR₂. In some embodiments, R¹¹ is —C(═O)R. In some embodiments, R¹¹ is —C(═O)OR. In some embodiments, R¹¹ is —C(═O)NR₂. In some embodiments, R¹¹ is —SO₂R. In some embodiments, R¹¹ is —SO₂NR₂. In some embodiments, R¹¹ is halogen. In some embodiments, R¹¹ is C₁₋₆haloalkyl. In some embodiments, R¹¹ is C₁₋₆haloalkoxy. In some embodiments, R¹¹ is an optionally substituted 3-8 membered saturated or partially unsaturated monocyclic carbocyclic ring. In some embodiments, R¹¹ is an optionally substituted 6-12 membered saturated or partially unsaturated bridged carbocyclic ring. In some embodiments, R¹¹ is an optionally substituted 7-12 membered saturated or partially unsaturated bicyclic carbocyclic ring. In some embodiments, R¹¹ is an optionally substituted phenyl. In some embodiments, R¹¹ is an optionally substituted 8-10 membered bicyclic aromatic carbocyclic ring. In some embodiments, R¹¹ is an optionally substituted 3-8 membered saturated or partially unsaturated monocyclic heterocyclic ring (having 1-2 heteroatoms independently selected from nitrogen, oxygen, and sulfur). In some embodiments, R¹¹ is an optionally substituted 6-12 membered saturated or partially unsaturated bridged heterocyclic ring (having 1-4 heteroatoms independently selected from nitrogen, oxygen, and sulfur). In some embodiments, R¹¹ is an optionally substituted 7-12 membered saturated or partially unsaturated bicyclic heterocyclic ring (having 1-4 heteroatoms independently selected from nitrogen, oxygen, and sulfur). In some embodiments, R¹¹ is an optionally substituted 5-6 membered monocyclic heteroaromatic ring (having 1-4 heteroatoms independently selected from nitrogen, oxygen, and sulfur). In some embodiments, R¹¹ is an optionally substituted 8-10 membered bicyclic heteroaromatic ring (having 1-5 heteroatoms independently selected from nitrogen, oxygen, and sulfur).

In some embodiments, R¹¹ is —OCF₃. In some embodiments, R¹¹ is cyclopropyl. In some embodiments, R¹¹ is cyclobutyl. In some embodiments, R¹¹ is optionally substituted pyrazolyl. In some embodiments, R¹¹ is optionally substituted pyridinyl. In some embodiments, R¹¹ is optionally substituted pyrimidinyl. In some embodiments, R¹¹ is optionally substituted pyridazinyl. In some embodiments, R¹¹ is optionally substituted imidazolyl. In some embodiments, R¹¹ is optionally substituted triazolyl. In some embodiments, R¹¹ is optionally substituted oxazolyl. In some embodiments, R¹¹ is optionally substituted thiazolyl. In some embodiments, R¹¹ is optionally substituted oxadiazolyl. In some embodiments, R¹¹ is optionally substituted thiadiazolyl. In some embodiments, R¹¹ is optionally substituted oxetanyl. In some embodiments, R¹¹ is optionally substituted azetidinyl. In some embodiments, R¹¹ is optionally substituted piperidinyl. In some embodiments, R¹¹ is optionally substituted piperazinyl. In some embodiments, R¹¹ is selected from those depicted in Table A below.

In some embodiments, R¹⁰ and R¹¹ are independently a substituent selected from hydrogen and those shown below:

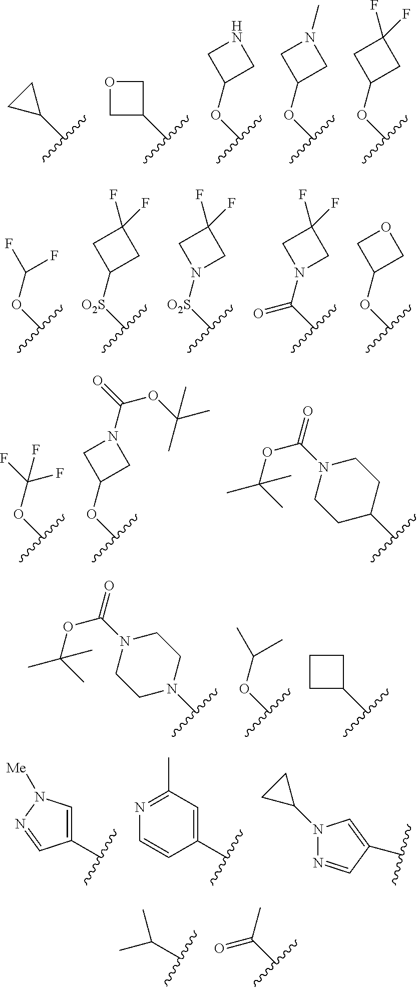

In some embodiments, R¹⁰ and R¹¹ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹⁰ and R¹¹ are taken together with their intervening atoms to form an optionally substituted 3-8 membered saturated or partially unsaturated monocyclic carbocyclic ring. In some embodiments, R¹⁰ and R¹¹ are taken together with their intervening atoms to form an optionally substituted 6-12 membered saturated or partially unsaturated bridged carbocyclic ring. In some embodiments, R¹⁰ and R¹¹ are taken together with their intervening atoms to form an optionally substituted 7-12 membered saturated or partially unsaturated bicyclic carbocyclic ring. In some embodiments, R¹⁰ and R¹¹ are taken together with their intervening atoms to form an optionally substituted phenyl. In some embodiments, R¹⁰ and R¹¹ are taken together with their intervening atoms to form an optionally substituted 8-10 membered bicyclic aromatic carbocyclic ring. In some embodiments, R¹⁰ and R¹¹ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R¹⁰ and R¹¹ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R¹⁰ and R¹¹ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R¹⁰ and R¹¹ are taken together with their intervening atoms to form an optionally substituted 5-6 membered monocyclic heteroaromatic ring (having 1-4 heteroatoms independently selected from nitrogen, oxygen, and sulfur). In some embodiments, R¹⁰ and R¹¹ are taken together with their intervening atoms to form an optionally substituted 8-10 membered bicyclic heteroaromatic ring (having 1-5 heteroatoms independently selected from nitrogen, oxygen, and sulfur).

In some embodiments, R¹⁰ and R¹¹ are taken together with their intervening atoms to form a dioxole ring.

In some embodiments, Ring B is

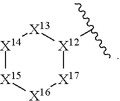

In some embodiments, Ring B is

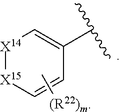

In some embodiments, Ring B is

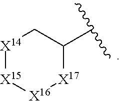

As defined generally above, X¹² is N, CH, or CR¹². In some embodiments, X¹² is N. In some embodiments, X¹² is CH. In some embodiments, X¹² is CCH₃. In some embodiments, X¹² is COH. In some embodiments, X¹² is CF. In some embodiments, X¹² is CR¹². In some embodiments, In some embodiments, X¹² is selected from those depicted in Table A below.

As defined generally above, X¹³ is O, NR¹³, C(R¹³)₂, CHR¹³, SO₂, or C═O. In some embodiments, X¹³ is O. In some embodiments, X¹³ is NR¹³. In some embodiments, X¹³ is C(R¹³)₂. In some embodiments, X¹³ is CHR¹³. In some embodiments, X¹³ is CH₂. In some embodiments, X¹³ is SO₂. In some embodiments, X¹³ is C═O. In some embodiments, X³ is selected from those depicted in Table A below.

As defined generally above, X¹⁴ is O, NR⁴, C(R¹⁴)₂, CHR¹⁴, SO₂, or C═O. In some embodiments, X¹⁴ is O. In some embodiments, X¹⁴ is NR¹⁴. In some embodiments, X¹⁴ is C(R¹⁴)₂. In some embodiments, X¹⁴ is CHR¹⁴. In some embodiments, X¹⁴ is CH₂. In some embodiments, X¹⁴ is SO₂. In some embodiments, X¹⁴ is C═O. In some embodiments, X¹⁴ is selected from those depicted in Table A below.

As defined generally above, X¹⁰ is O, NR¹⁶, C(R¹⁶)₂, CHR¹⁶, SO₂, or C═O. In some embodiments, X¹⁰ is O. In some embodiments, X¹⁰ is NR”. In some embodiments, X¹⁰ is C(R¹⁶)₂. In some embodiments, X¹⁰ is CHR¹⁶. In some embodiments, X¹⁰ is SO₂. In some embodiments, X¹⁰ is C═O. In some embodiments, X¹⁰ is CH₂, CF₂, or O. In some embodiments, X¹⁰ is CH₂. In some embodiments, X¹⁰ is NR¹⁰, or O. In some embodiments, X¹⁰ is NMe, NH, or O. In some embodiments, X¹⁰ is selected from those depicted in Table A below.

As defined generally above, X¹⁶ is O, NR¹⁶, C(R¹⁶)₂, CHR¹⁶, SO₂, or C═O. In some embodiments, X¹⁶ is O. In some embodiments, X¹⁶ is NR¹⁶. In some embodiments, X¹⁶ is C(R¹⁶)₂. In some embodiments, X¹⁶ is CHR¹⁶. In some embodiments, X¹⁶ is SO₂. In some embodiments, X¹⁶ is C═O. In some embodiments, X¹⁶ is CH₂. In some embodiments, X¹⁶ is selected from those depicted in Table A below.

As defined generally above, X¹⁷ is a direct bond, O, NR¹⁷, C(R¹⁷)₂, CHR¹⁷, —CH₂CH₂—, —OCH₂—, SO₂, or C═O. In some embodiments, X¹⁷ is O. In some embodiments, X¹⁷ is NR¹⁷. In some embodiments, X¹⁷ is C(R¹⁷)₂. In some embodiments, X¹⁷ is CHR¹⁷. In some embodiments, X¹⁷ is SO₂. In some embodiments, X¹⁷ is C═O. In some embodiments, X¹⁷ is —CH₂CH₂—. In some embodiments, X¹⁷ is —OCH₂—. In some embodiments, X¹⁷ is CH₂. In some embodiments, X¹⁷ is a direct bond. In some embodiments, X¹⁷ is selected from those depicted in Table A below.

In some embodiments, when any of X¹², X¹³, X¹⁴, X¹⁵, X¹⁶, or X¹⁷ is N, O or SO₂, then neither of the neighboring positions in Ring B are N, O or SO₂.

In some embodiments, when any one of X¹³, X¹⁴, X¹, X¹⁶, or X¹⁷ is C═O, then neither of the neighboring positions in Ring B are C═O or SO₂.

As defined generally above, R¹² is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R¹² is an optionally substituted aliphatic group. In some embodiments, R¹² is halogen. In some embodiments, R¹² is —OR. In some embodiments, R¹² is —NR₂. In some embodiments, R¹² is —C(═O)R. In some embodiments, R¹² is —C(═O)OR. In some embodiments, R¹² is —C(═O)NR₂. In some embodiments, R¹² is —SO₂R. In some embodiments, R¹² is —SO₂NR₂. In some embodiments, R¹² is C₁₋₆haloalkyl. In some embodiments, R¹² is C₁₋₆haloalkoxy. In some embodiments, R¹² is methyl. In some embodiments, R¹² is OH. In some embodiments, R¹² is F. In some embodiments, R¹² is selected from those depicted in Table A below.

As defined generally above, each of R¹³, R¹⁴, R¹⁵, R¹⁶, and R¹⁷ is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or any two of R², R¹³, R¹⁴, R¹, R¹⁶, and R¹⁷ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹³ is hydrogen. In some embodiments, R¹³ is an optionally substituted C₁₋₆ aliphatic group. In some embodiments, R¹³ —OR. In some embodiments, R¹³ is —NR₂. In some embodiments, R¹³ is —C(═O)R. In some embodiments, R¹³ is —C(═O)OR. In some embodiments, R¹³ is —C(═O)NR₂. In some embodiments, R¹³ is —SO₂R. In some embodiments, R¹³ is —SO₂NR₂. In some embodiments, R¹³ is C₁₋₆haloalkyl. In some embodiments, R¹³ is C₁₋₆haloalkoxy. In some embodiments, R¹³ is an optionally substituted 3-8 membered saturated or partially unsaturated monocyclic carbocyclic ring. In some embodiments, R¹³ is an optionally substituted 7-12 membered saturated or partially unsaturated bicyclic carbocyclic ring. In some embodiments, R¹³ is an optionally substituted phenyl. In some embodiments, R¹³ is an optionally substituted 8-10 membered bicyclic aromatic carbocyclic ring. In some embodiments, R¹³ is an optionally substituted 3-8 membered saturated or partially unsaturated monocyclic heterocyclic ring (having 1-2 heteroatoms independently selected from nitrogen, oxygen, and sulfur). In some embodiments, R¹³ is an optionally substituted 7-12 membered saturated or partially unsaturated bicyclic heterocyclic ring (having 1-4 heteroatoms independently selected from nitrogen, oxygen, and sulfur). In some embodiments, R¹³ is an optionally substituted 5-6 membered monocyclic heteroaromatic ring (having 1-4 heteroatoms independently selected from nitrogen, oxygen, and sulfur). In some embodiments, R¹³ is an optionally substituted 8-10 membered bicyclic heteroaromatic ring (having 1-5 heteroatoms independently selected from nitrogen, oxygen, and sulfur). In some embodiments, R¹³ is methyl. In some embodiments, R³ is —OH. In some embodiments, R³ is F. In some embodiments, R¹³ is methoxy. In some embodiments, R³ is —CH₂OH. In some embodiments, wherein X¹³ is C(R¹³)₂, each R¹³ is independently selected from any of the aforementioned substituents. In some embodiments, wherein X¹³ is C(R¹³)₂, both R¹³ are the same. In some embodiments, R¹³ is selected from those depicted in Table A below.

In some embodiments, R¹⁴ is hydrogen. In some embodiments, R¹⁴ is an optionally substituted C₁₋₆ aliphatic group. In some embodiments, R¹⁴ —OR. In some embodiments, R¹⁴ is —NR₂. In some embodiments, R¹⁴ is —C(═O)R. In some embodiments, R¹⁴ is —C(═O)OR. In some embodiments, R¹⁴ is —C(═O)NR₂. In some embodiments, R¹⁴ is —SO₂R. In some embodiments, R¹⁴ is —SO₂NR₂. In some embodiments, R¹⁴ is C₁₋₆haloalkyl. In some embodiments, R¹⁴ is C₁₋₆haloalkoxy. In some embodiments, R¹⁴ is an optionally substituted 3-8 membered saturated or partially unsaturated monocyclic carbocyclic ring. In some embodiments, R¹⁴ is an optionally substituted 7-12 membered saturated or partially unsaturated bicyclic carbocyclic ring. In some embodiments, R¹⁴ is an optionally substituted phenyl. In some embodiments, R¹⁴ is an optionally substituted 8-10 membered bicyclic aromatic carbocyclic ring. In some embodiments, R¹⁴ is an optionally substituted 3-8 membered saturated or partially unsaturated monocyclic heterocyclic ring (having 1-2 heteroatoms independently selected from nitrogen, oxygen, and sulfur). In some embodiments, R¹⁴ is an optionally substituted 7-12 membered saturated or partially unsaturated bicyclic heterocyclic ring (having 1-4 heteroatoms independently selected from nitrogen, oxygen, and sulfur). In some embodiments, R¹⁴ is an optionally substituted 5-6 membered monocyclic heteroaromatic ring (having 1-4 heteroatoms independently selected from nitrogen, oxygen, and sulfur). In some embodiments, R¹⁴ is an optionally substituted 8-10 membered bicyclic heteroaromatic ring (having 1-5 heteroatoms independently selected from nitrogen, oxygen, and sulfur).

In some embodiments, R¹⁴ is optionally substituted pyrazolyl. In some embodiments, R¹⁴ is optionally substituted pyridinyl. In some embodiments, R¹⁴ is optionally substituted pyrimidinyl. In some embodiments, R¹⁴ is optionally substituted pyridazinyl. In some embodiments, R¹⁴ is optionally substituted imidazolyl. In some embodiments, R¹⁴ is optionally substituted triazolyl. In some embodiments, R¹⁴ is optionally substituted oxazolyl. In some embodiments, R¹⁴ is optionally substituted thiazolyl. In some embodiments, R¹⁴ is optionally substituted oxadiazolyl. In some embodiments, R¹⁴ is optionally substituted thiadiazolyl. In some embodiments, R¹⁴ is optionally substituted oxetanyl. In some embodiments, R¹⁴ is optionally substituted azetidinyl. In some embodiments, R¹⁴ is optionally substituted piperidinyl. In some embodiments, R¹⁴ is optionally substituted piperazinyl. In some embodiments, R¹⁴ is methyl. In some embodiments, R¹⁴ is —OH. In some embodiments, R¹⁴ is F. In some embodiments, R¹⁴ is methoxy. In some embodiments, R¹⁴ is —CH₂OH. In some embodiments, wherein X¹⁴ is C(R¹⁴)₂, each R¹⁴ is independently selected from any of the aforementioned substituents. In some embodiments, wherein X¹⁴ is C(R¹⁴)₂, both R¹⁴ are the same. In some embodiments, R¹⁴ is selected from those depicted in Table A below.

In some embodiments, R¹⁴ is substituted with an optionally substituted 3-6 membered saturated or partially unsaturated monocyclic carbocyclic ring. In some embodiments, R¹⁴ is substituted with an optionally substituted 5-8 membered saturated or partially unsaturated bicyclic carbocyclic ring. In some embodiments, R¹⁴ is substituted with an optionally susbstituted 3-6 membered saturated or partially unsaturated monocyclic heterocyclic ring. In some embodiments, R¹⁴ is substituted with an optionally substituted C₁₋₆ aliphatic group. In some embodiments, R¹⁴ is substituted with a methyl group. In some embodiments, R¹⁴ is substituted with a —CD₃ group. In some embodiments, R¹⁴ is substituted with a methoxy group. In some embodiments, R¹⁴ is substituted with a cyclopropyl group. In some embodiments, R¹⁴ is substituted with an optionally substituted

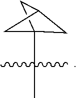 [SMILES: *C12CC(C1)C2]

In some embodiments, R¹⁴ is —OR, wherein R is an optionally substituted 5-6 membered heteroaryl ring (having 1-4 heteroatoms independently selected from nitrogen, oxygen, and sulfur). In some embodiments, R¹⁴ is —NHR, wherein R is an optionally substituted 5-6 membered heteroaryl ring (having 1-4 heteroatoms independently selected from nitrogen, oxygen, and sulfur). In some embodiments, R¹⁴ is —N(CH₃)R, wherein R is an optionally substituted 5-6 membered heteroaryl ring (having 1-4 heteroatoms independently selected from nitrogen, oxygen, and sulfur). In some embodiments, R¹⁴ is —C(═O)N(CH₃)R, wherein R is an optionally substituted 5-6 membered heteroaryl ring (having 1-4 heteroatoms independently selected from nitrogen, oxygen, and sulfur). In some embodiments, R¹⁴ is —C(═O)NHR, wherein R is an optionally substituted 5-6 membered heteroaryl ring (having 1-4 heteroatoms independently selected from nitrogen, oxygen, and sulfur).

In some embodiments, R¹⁴ is a substituent selected from those shown below:

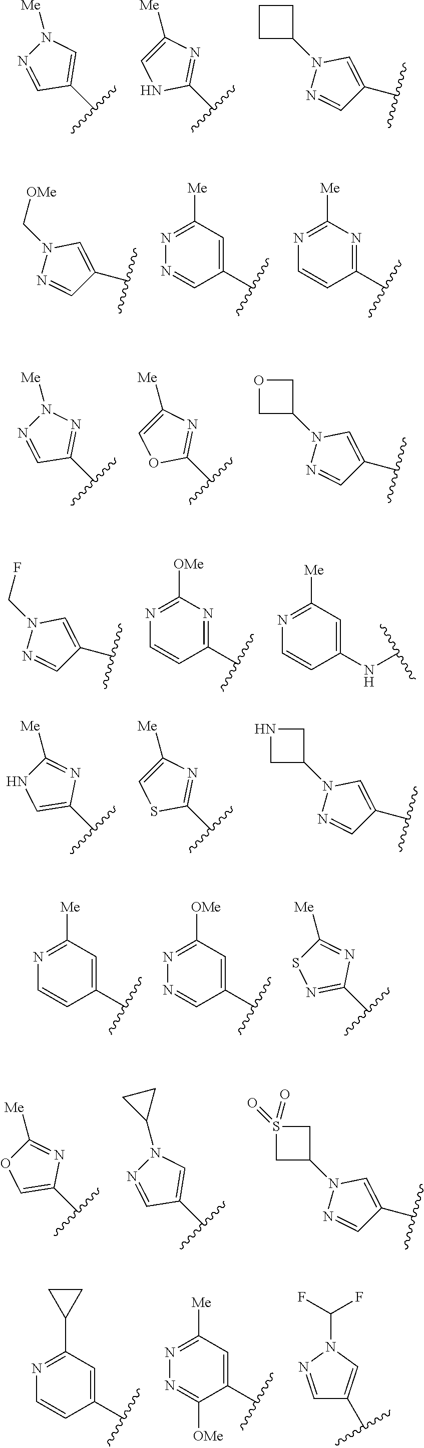

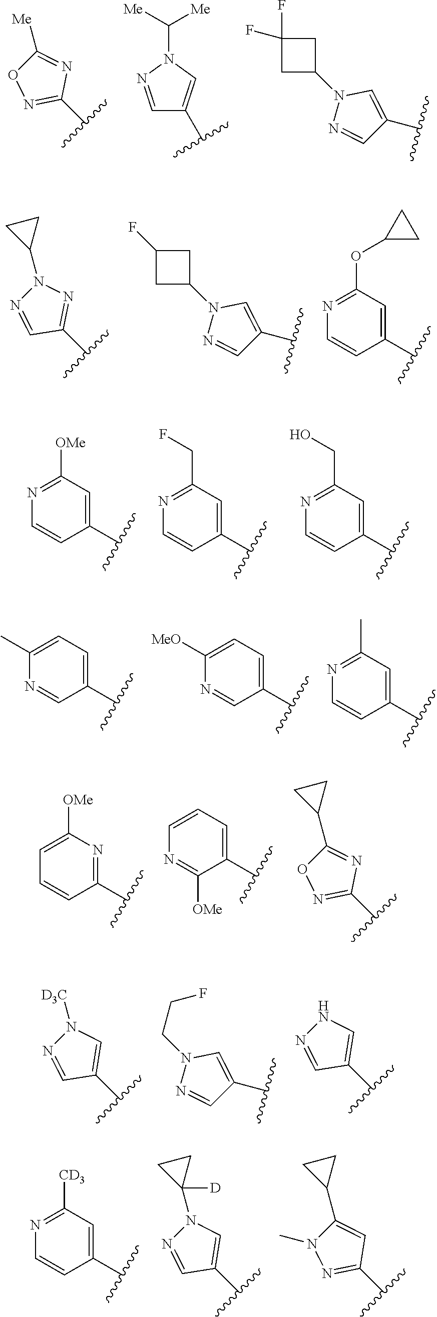

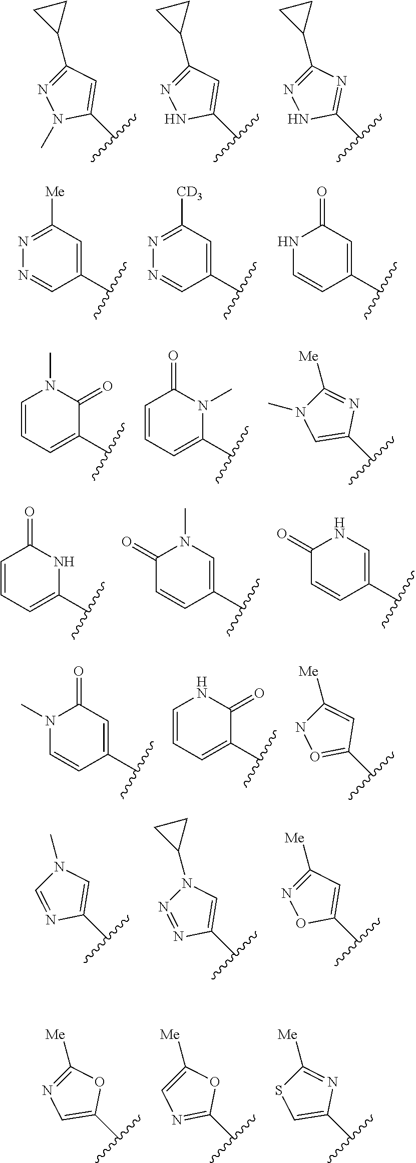

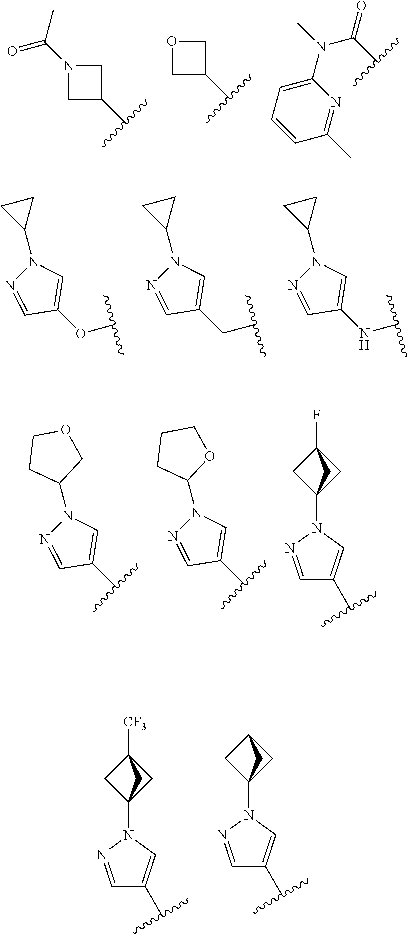

In some embodiments, R¹⁴ is methyl, tetrahydrofuran-3-yl,

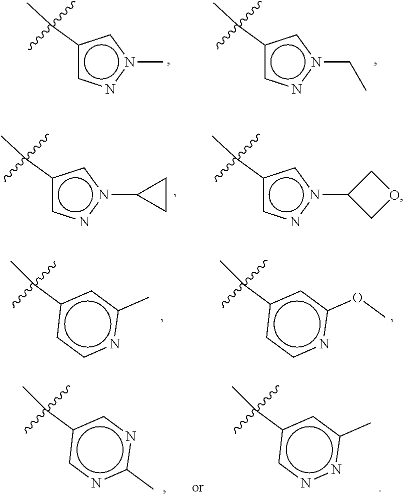

In some embodiments, R¹⁴ is methyl, tetrahydrofuran-3-yl,

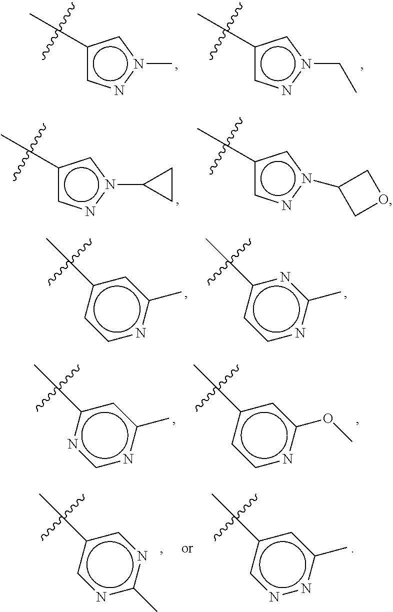

In some embodiments, R¹⁴ is

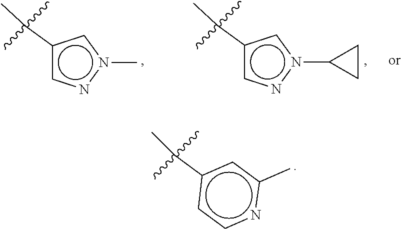

In some embodiments, R¹⁴ is

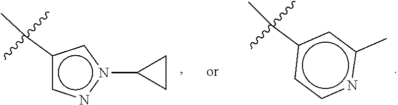

In some embodiments, R¹⁴ is

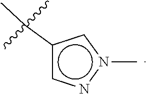

In some embodiments, R¹⁴ is

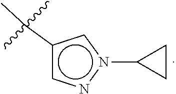

In some embodiments, R¹⁴ is

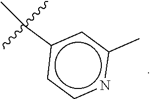

In some embodiments, R¹⁵ is hydrogen. In some embodiments, R¹⁵ is an optionally substituted C₁₋₆ aliphatic group. In some embodiments, R¹⁵ —OR. In some embodiments, R¹⁵ is —NR₂. In some embodiments, R¹⁵ is —C(═O)R. In some embodiments, R¹⁵ is —C(═O)OR. In some embodiments, R¹⁵ is —C(═O)NR₂. In some embodiments, R¹⁵ is —SO₂R. In some embodiments, R¹⁵ is —SO₂NR₂. In some embodiments, R¹⁵ is C₁₋₆haloalkyl. In some embodiments, R¹⁵ is C₁₋₆haloalkoxy. In some embodiments, R¹⁵ is an optionally substituted 3-8 membered saturated or partially unsaturated monocyclic carbocyclic ring. In some embodiments, R¹⁵ is an optionally substituted 7-12 membered saturated or partially unsaturated bicyclic carbocyclic ring. In some embodiments, R¹⁵ is an optionally substituted phenyl. In some embodiments, R¹⁵ is an optionally substituted 8-10 membered bicyclic aromatic carbocyclic ring. In some embodiments, R¹⁵ is an optionally substituted 3-8 membered saturated or partially unsaturated monocyclic heterocyclic ring (having 1-2 heteroatoms independently selected from nitrogen, oxygen, and sulfur). In some embodiments, R¹⁵ is an optionally substituted 7-12 membered saturated or partially unsaturated bicyclic heterocyclic ring (having 1-4 heteroatoms independently selected from nitrogen, oxygen, and sulfur). In some embodiments, R¹⁵ is an optionally substituted 5-6 membered monocyclic heteroaromatic ring (having 1-4 heteroatoms independently selected from nitrogen, oxygen, and sulfur). In some embodiments, R¹⁵ is an optionally substituted 8-10 membered bicyclic heteroaromatic ring (having 1-5 heteroatoms independently selected from nitrogen, oxygen, and sulfur). In some embodiments, R¹⁵ is methyl. In some embodiments, R¹⁵ is —OH. In some embodiments, R¹⁵ is F. In some embodiments, R¹⁵ is methoxy. In some embodiments, R¹⁵ is —CH₂OH. In some embodiments, wherein X¹⁵ is C(R¹⁵)₂, each R¹⁵ is independently selected from any of the aforementioned substituents. In some embodiments, wherein X¹⁵ is C(R¹⁵)₂, both R¹⁵ are the same. In some embodiments, R¹⁵ is selected from those depicted in Table A below.

In some embodiments, R¹⁶ is hydrogen. In some embodiments, R¹⁶ is an optionally substituted C₁₋₆ aliphatic group. In some embodiments, R¹⁶ —OR. In some embodiments, R¹⁶ is —NR₂. In some embodiments, R¹⁶ is —C(═O)R. In some embodiments, R¹⁶ is —C(═O)OR. In some embodiments, R¹⁶ is —C(═O)NR₂. In some embodiments, R¹⁶ is —SO₂R. In some embodiments, R¹⁶ is —SO₂NR₂. In some embodiments, R¹⁶ is C₁₋₆haloalkyl. In some embodiments, R¹⁶ is C₁₋₆haloalkoxy. In some embodiments, R¹⁶ is an optionally substituted 3-8 membered saturated or partially unsaturated monocyclic carbocyclic ring. In some embodiments, R¹⁶ is an optionally substituted 7-12 membered saturated or partially unsaturated bicyclic carbocyclic ring. In some embodiments, R¹⁶ is an optionally substituted phenyl. In some embodiments, R¹⁶ is an optionally substituted 8-10 membered bicyclic aromatic carbocyclic ring. In some embodiments, R¹⁶ is an optionally substituted 3-8 membered saturated or partially unsaturated monocyclic heterocyclic ring (having 1-2 heteroatoms independently selected from nitrogen, oxygen, and sulfur). In some embodiments, R¹⁶ is an optionally substituted 7-12 membered saturated or partially unsaturated bicyclic heterocyclic ring (having 1-4 heteroatoms independently selected from nitrogen, oxygen, and sulfur). In some embodiments, R¹⁶ is an optionally substituted 5-6 membered monocyclic heteroaromatic ring (having 1-4 heteroatoms independently selected from nitrogen, oxygen, and sulfur). In some embodiments, R¹⁶ is an optionally substituted 8-10 membered bicyclic heteroaromatic ring (having 1-5 heteroatoms independently selected from nitrogen, oxygen, and sulfur). In some embodiments, R¹⁶ is methyl. In some embodiments, R¹⁶ is —OH. In some embodiments, R¹⁶ is F. In some embodiments, R¹⁶ is methoxy. In some embodiments, R¹⁶ is —CH₂OH. In some embodiments, wherein X¹⁶ is C(R¹⁶)₂, each R¹⁶ is independently selected from any of the aforementioned substituents. In some embodiments, wherein X¹⁶ is C(R¹⁶)₂, both R¹⁶ are the same. In some embodiments, R¹⁶ is selected from those depicted in Table A below.

In some embodiments, R¹⁷ is hydrogen. In some embodiments, R¹⁷ is an optionally substituted C₁₋₆ aliphatic group. In some embodiments, R¹⁷ —OR. In some embodiments, R¹⁷ is —NR₂. In some embodiments, R¹⁷ is —C(═O)R. In some embodiments, R¹⁷ is —C(═O)OR. In some embodiments, R¹⁷ is —C(═O)NR₂. In some embodiments, R¹⁷ is —SO₂R. In some embodiments, R¹⁷ is —SO₂NR₂. In some embodiments, R¹⁷ is C₁₋₆haloalkyl. In some embodiments, R¹⁷ is C₁₋₆haloalkoxy. In some embodiments, R¹⁷ is an optionally substituted 3-8 membered saturated or partially unsaturated monocyclic carbocyclic ring. In some embodiments, R¹⁷ is an optionally substituted 7-12 membered saturated or partially unsaturated bicyclic carbocyclic ring. In some embodiments, R¹⁷ is an optionally substituted phenyl. In some embodiments, R¹⁷ is an optionally substituted 8-10 membered bicyclic aromatic carbocyclic ring. In some embodiments, R¹⁷ is an optionally substituted 3-8 membered saturated or partially unsaturated monocyclic heterocyclic ring (having 1-2 heteroatoms independently selected from nitrogen, oxygen, and sulfur). In some embodiments, R¹⁷ is an optionally substituted 7-12 membered saturated or partially unsaturated bicyclic heterocyclic ring (having 1-4 heteroatoms independently selected from nitrogen, oxygen, and sulfur). In some embodiments, R¹⁷ is an optionally substituted 5-6 membered monocyclic heteroaromatic ring (having 1-4 heteroatoms independently selected from nitrogen, oxygen, and sulfur). In some embodiments, R¹⁷ is an optionally substituted 8-10 membered bicyclic heteroaromatic ring (having 1-5 heteroatoms independently selected from nitrogen, oxygen, and sulfur). In some embodiments, R¹⁷ is methyl. In some embodiments, R¹⁷ is —OH. In some embodiments, R¹⁷ is F. In some embodiments, R¹⁷ is methoxy. In some embodiments, R¹⁷ is —CH₂OH. In some embodiments, wherein X¹⁷ is C(R¹⁷)₂, each R¹⁷ is independently selected from any of the aforementioned substituents. In some embodiments, wherein X¹⁷ is C(R¹⁷)₂, both R¹⁷ are the same. In some embodiments, R¹⁷ is selected from those depicted in Table A below.

In some embodiments, Ring B is a substituent selected from those shown below:

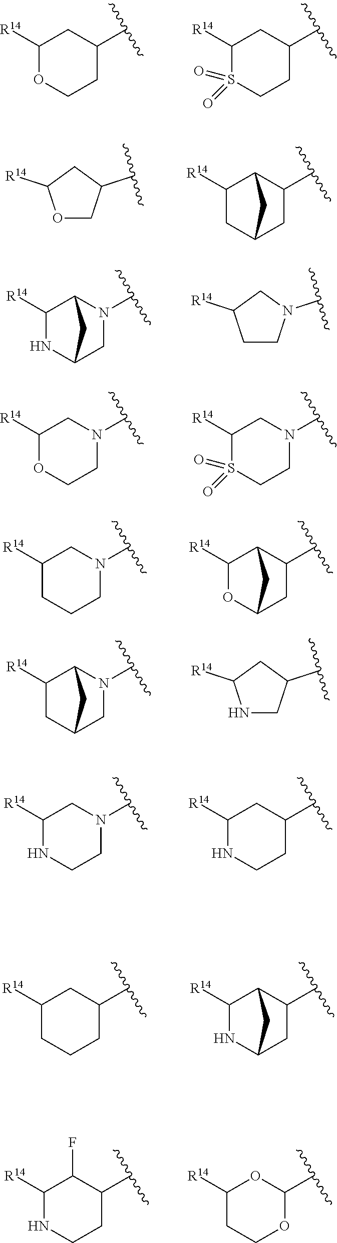

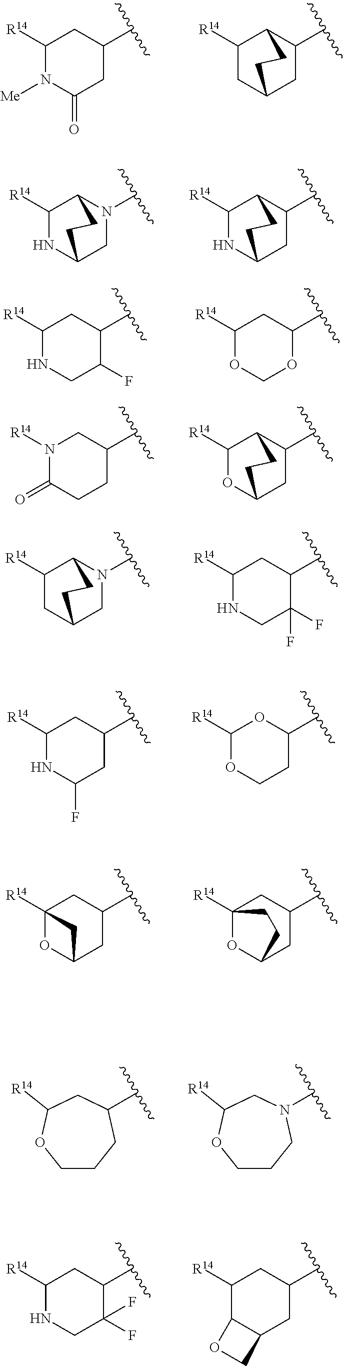

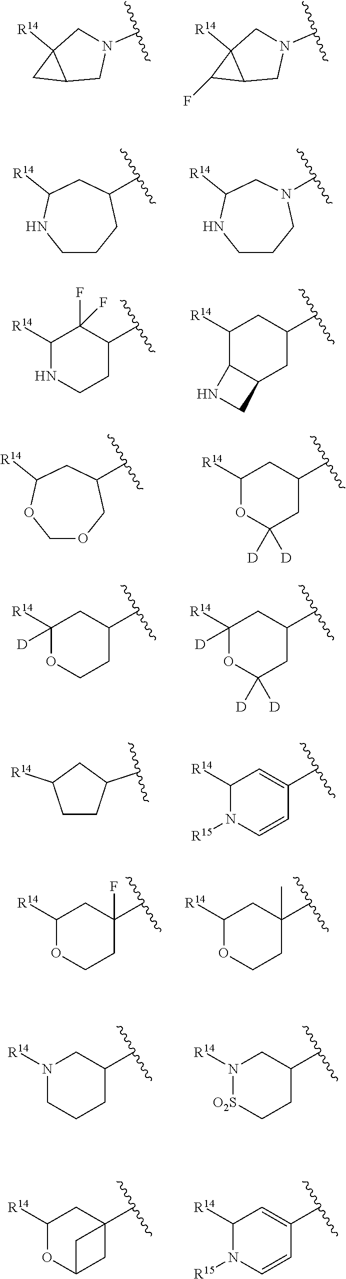

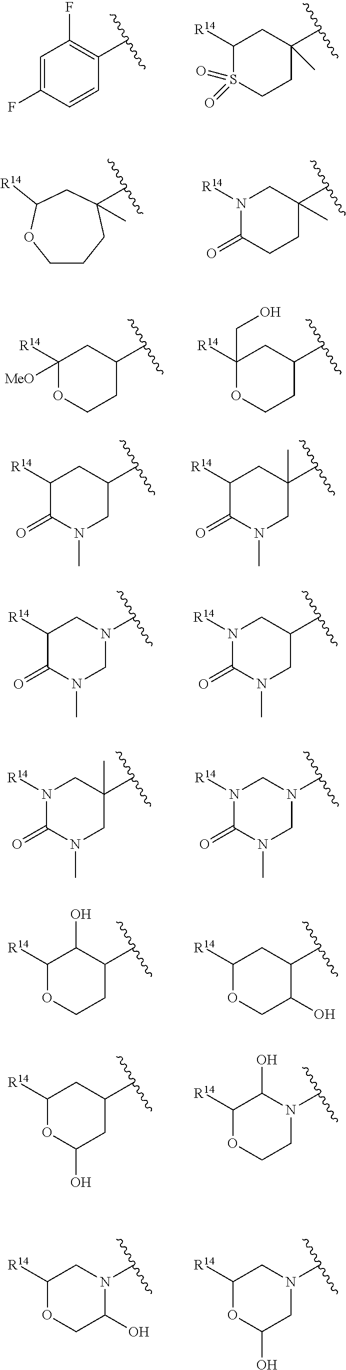

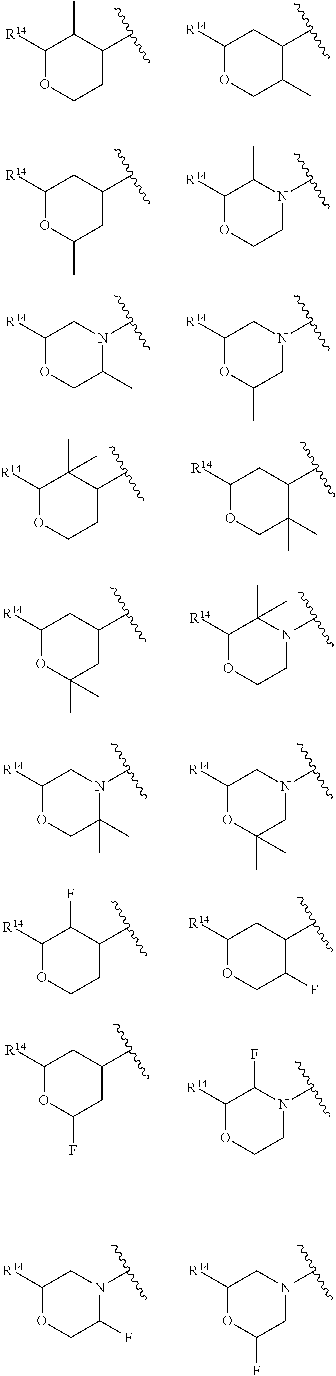

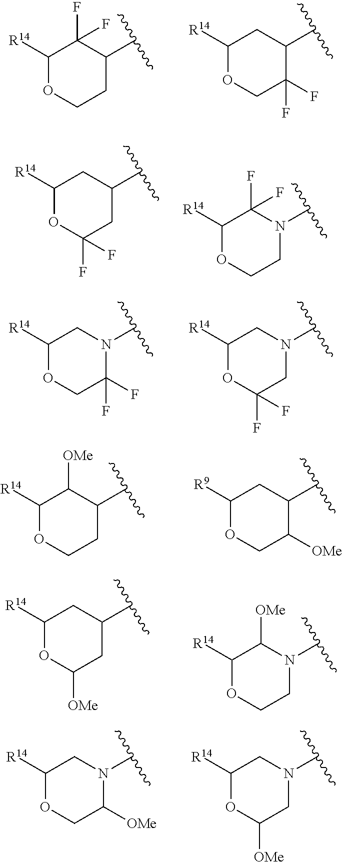

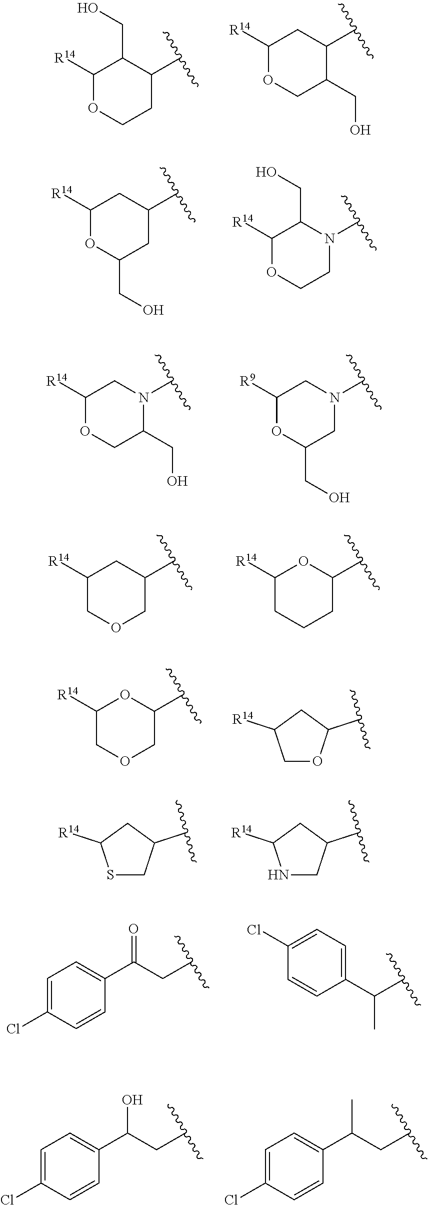

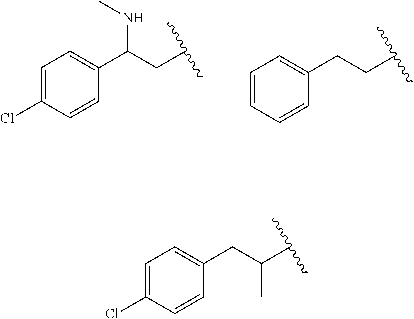 [SMILES: *CC(NC)c1ccc(Cl)cc1 ; *CCc1ccccc1 ; *C(C)Cc1ccc(Cl)cc1]

In some embodiments, Ring B is

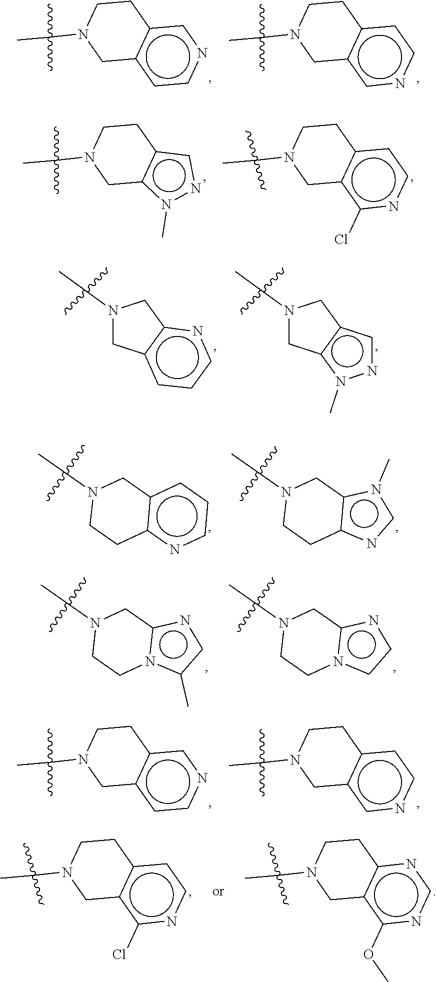

In some embodiments, Ring B is

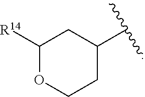

In some embodiments, Ring B is

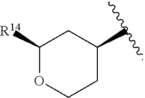

In some embodiments, Ring B is

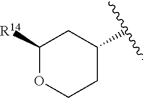

In some embodiments, Ring B is

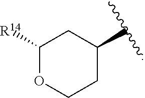

In some embodiments, Ring B is

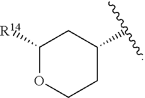

In some embodiments, Ring B is

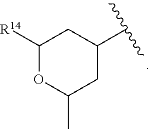

In some embodiments, Ring B is

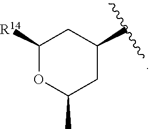

In some embodiments, Ring B is

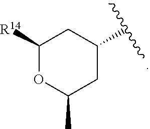

In some embodiments, Ring B is

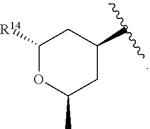

In some embodiments, Ring B is

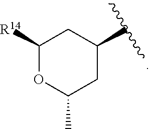

In some embodiments, Ring B is

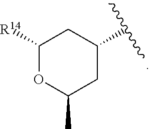

In some embodiments, Ring B is

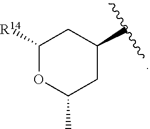

In some embodiments, Ring B is

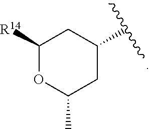

In some embodiments, Ring B is

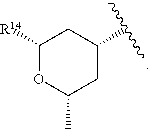

In some embodiments, Ring B is

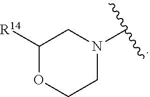

In some embodiments, Ring B is

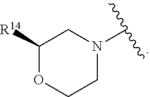

In some embodiments, Ring B is

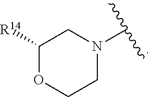

In some embodiments, Ring B is

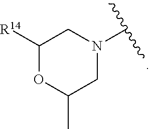

In some embodiments, Ring B is

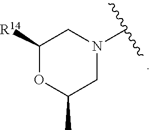

In some embodiments, Ring B is

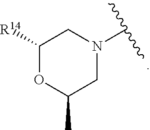

In some embodiments, Ring B is

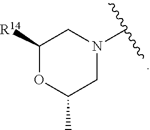

In some embodiments, Ring B is

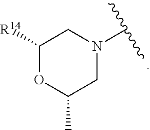

In some embodiments, Ring B is

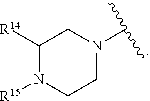

In some embodiments, Ring B is

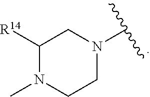

In some embodiments, Ring B is

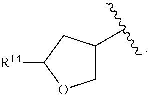

In some embodiments, Ring B is

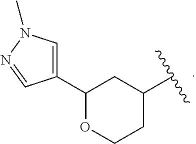 [SMILES: *C1CCOC(c2cnn(C)c2)C1]

In some embodiments, Ring B is

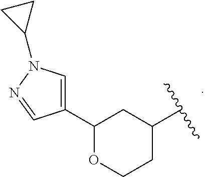 [SMILES: *C1CCOC(c2cnn(C3CC3)c2)C1]

In some embodiments, Ring B is

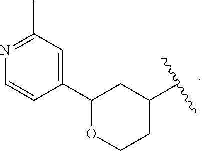 [SMILES: *C1CCOC(c2ccnc(C)c2)C1]

In some embodiments, Ring B is

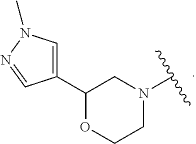 [SMILES: *N1CCOC(c2cnn(C)c2)C1]

In some embodiments, Ring B is

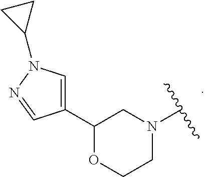 [SMILES: *N1CCOC(c2cnn(C3CC3)c2)C1]

In some embodiments, Ring B is

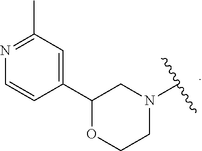 [SMILES: *N1CCOC(c2ccnc(C)c2)C1]

In some embodiments, Ring B is

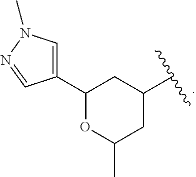 [SMILES: *C1CC(C)OC(c2cnn(C)c2)C1]

some embodiments, Ring B is

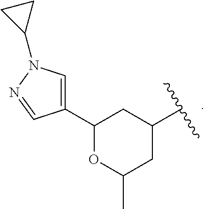 [SMILES: *C1CC(C)OC(c2cnn(C3CC3)c2)C1]

In some embodiments, Ring B is

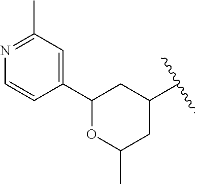

In some embodiments, Ring B is

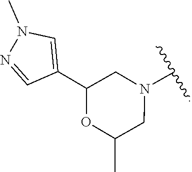

In some embodiments, Ring B is

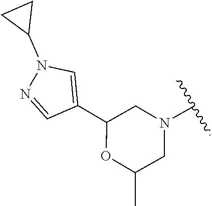

In some embodiments, Ring B is

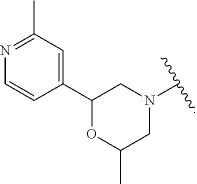

In some embodiments, Ring B is

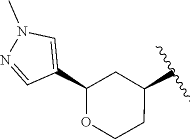

In some embodiments, Ring B is

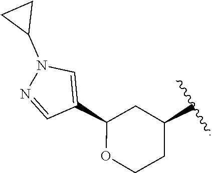

In some embodiments, Ring B is

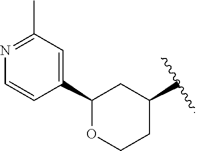

In some embodiments, Ring B is

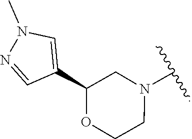

In some embodiments, Ring B is

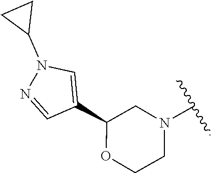

In some embodiments, Ring B is

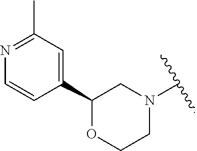

In some embodiments, Ring B is

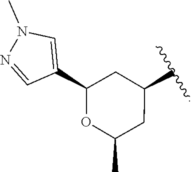

In some embodiments, Ring B is

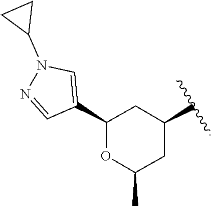

In some embodiments, Ring B is

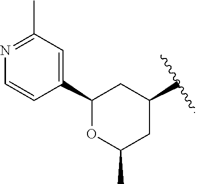

In some embodiments, Ring B is

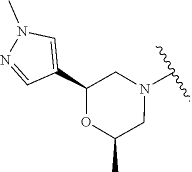

In some embodiments, Ring B is

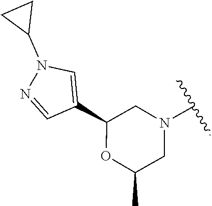

In some embodiments, Ring B is

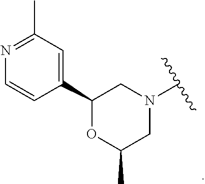 [SMILES: *N1C[C@@H](C)O[C@@H](c2ccnc(C)c2)C1]

In some embodiments, R² is H or methyl; R⁴ is

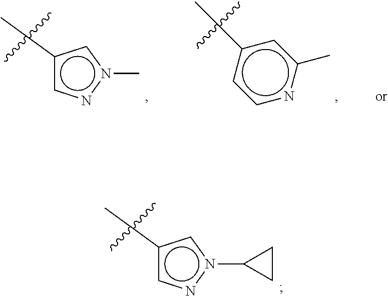

### R⁵ is

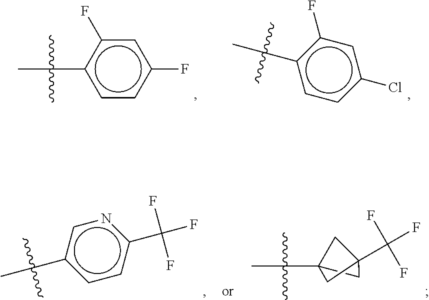

R⁶ is H or methyl and R⁷ is methyl.

In some embodiments, R² is H or methyl; R⁴ is

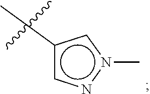

### R⁵ is

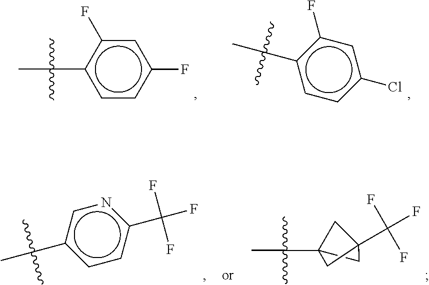

R⁶ is H or methyl and R⁷ is methyl.

In some embodiments, R² is H or methyl; R⁴ is

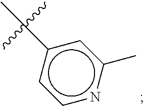

### R⁵ is

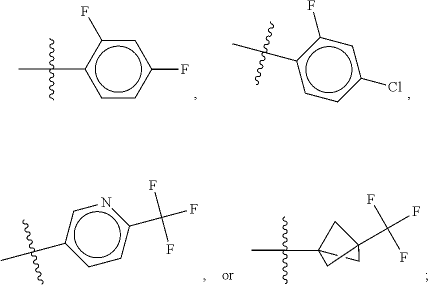

R⁶ is H or methyl and R⁷ is methyl.

In some embodiments, R² is H or methyl; R⁴ is

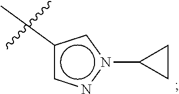

### R⁵ is

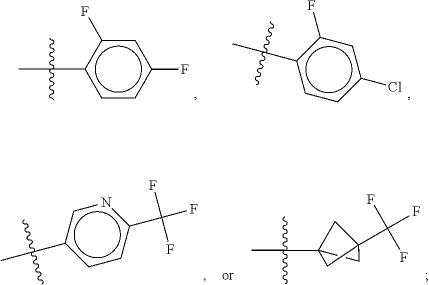

R⁶ is H or methyl and R⁷ is methyl.

In some embodiments, R² is H or methyl; R⁴ is

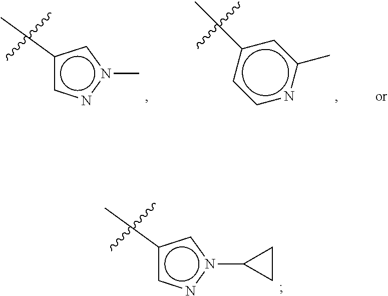

### R⁵ is

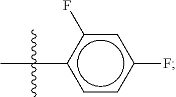

R⁶ is H or methyl and R⁷ is methyl.

In some embodiments, R² is H or methyl; R⁴ is

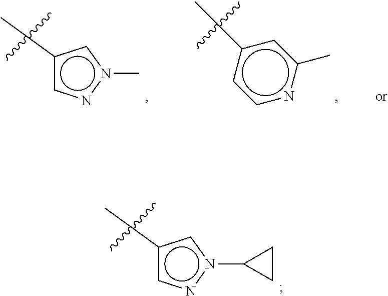

### R⁵ is

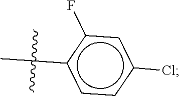

R⁶ is H or methyl and R⁷ is methyl.

In some embodiments, R² is H or methyl; R⁴ is

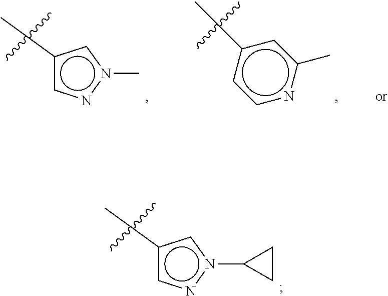

### R⁵ is

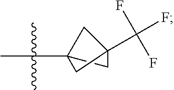 [SMILES: *C12CC(C(F)(F)F)(C1)C2]

R⁶ is H or methyl and R⁷ is methyl.

In some embodiments, R² is H or methyl; R⁴ is

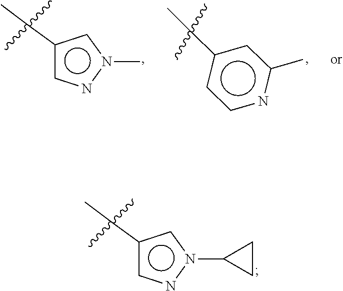

### R⁵ is

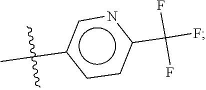

R⁶ is H or methyl and R⁷ is methyl.

In some embodiments, R² is H or methyl; R⁴ is

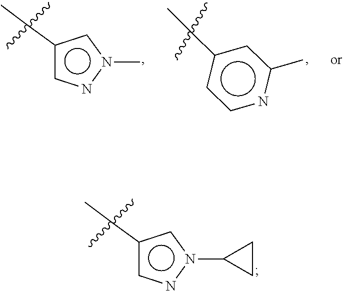

### R⁵ is

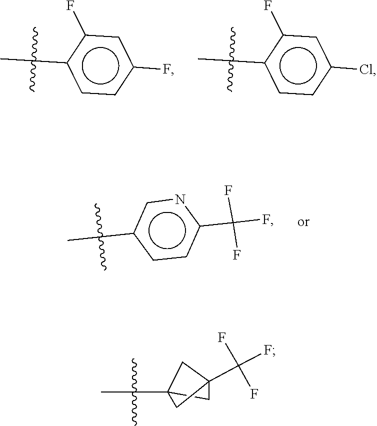

and R⁹ is methyl, ethyl or iso-propyl.

In some embodiments, at least one hydrogen atom of the compound is a deuterium atom. In some embodiments, at least one C₁-C₆alkyl group of the compound is substituted with at least one deuterium atom. In some embodiments, R⁶ is —CD₃. In some embodiments, R⁷ is —CD₃. In some embodiments, R⁶ and R⁷ are both —CD₃. In some embodiments, R⁶ and R⁷ are each independently selected from H, D, —CH₃, —CD₃, —CHD₂, and —CH₂D. In some embodiments, R⁶ and R⁷ are each independently selected from —CH₃, —CD₃, —CHD₂, and —CH₂D. In some embodiments, R² is deuterium. In some embodiments, the hydrogen atom attached to the same carbon as R² is deuterium. In some embodiments, R⁴ is substituted with C₁₋₃alkyl, comprising one or more deuteriums. In some embodiments, R⁴ is substituted with 1 to 3 substitutents selected from —CD₃, —CHD₂, and —CH₂D.

In some embodiments, the compound is a compound of Formula IIIa

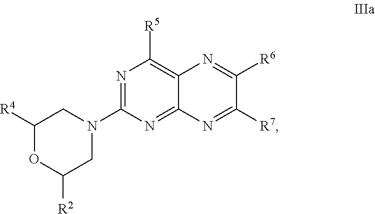

or a pharmaceutically acceptable salt thereof;
wherein
   R² is H or methyl;
   R⁴ is

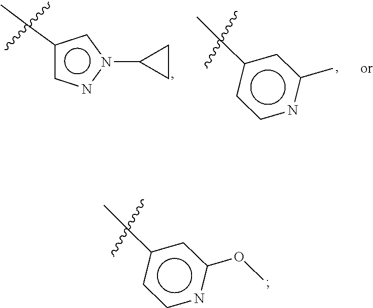

R⁵ is C₃₋₆cycloalkyl, C₅₋₈ spiroalkyl, C₅₋₈ tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that:
when R⁴ is

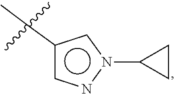

and R² is H, R⁵ is not

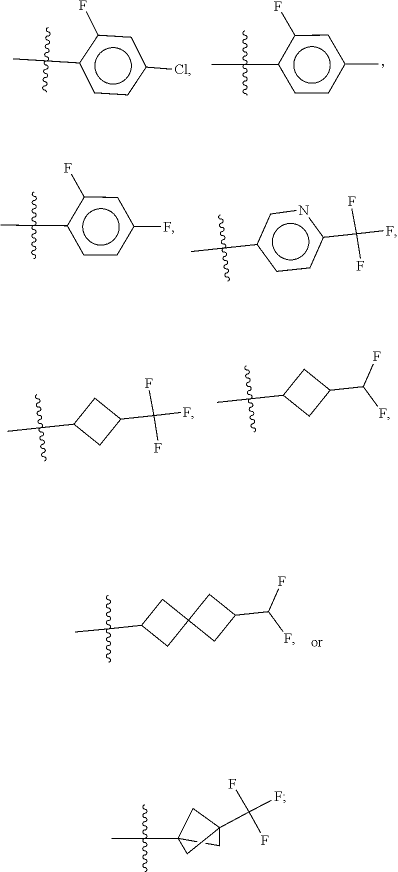

and
when R⁴ is

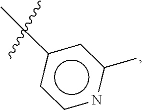

and R² is H, R⁵ is not

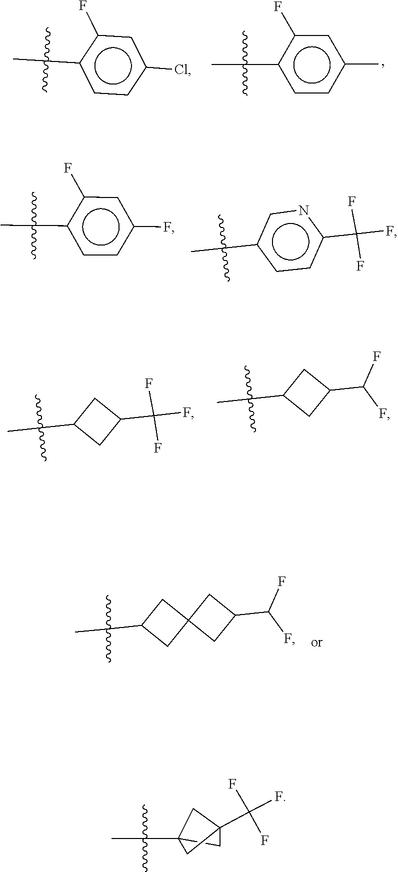

In some embodiments, the compound is a compound of Formula IIIa

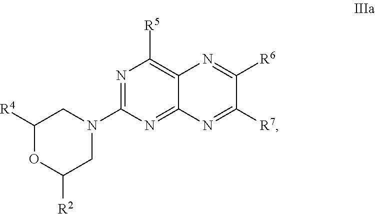

or a pharmaceutically acceptable salt thereof;
wherein
   R² is H or methyl;
   R⁴ is

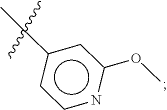

R⁵ is C₃₋₆cycloalkyl, C₅₋₈ spiroalkyl, C₅₋₈ stricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl.

In some embodiments, the compound is a compound of Formula IIIa

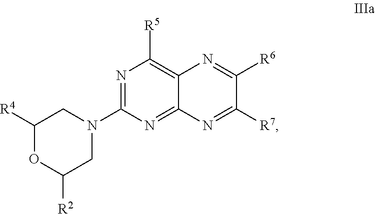

or a pharmaceutically acceptable salt thereof;
wherein
   R² is methyl;
   R⁴ is

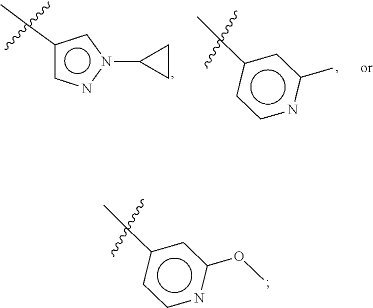

R⁵ is C₃₋₆cycloalkyl, C₅₋₈ spiroalkyl, C₅₋₈ tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is Me.

In some embodiments, the compound is a compound of Formula IIIa

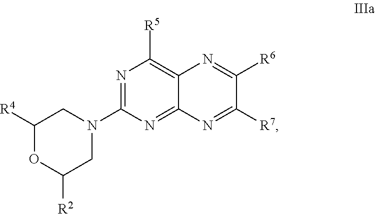

or a pharmaceutically acceptable salt thereof;
wherein
   R² is H or methyl;
   R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
   R⁵ is

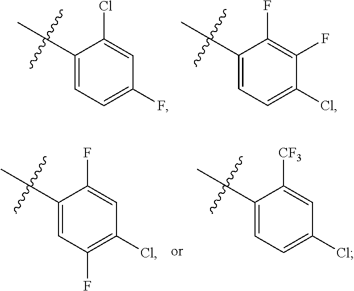 [SMILES: *c1ccc(F)cc1Cl ; *c1ccc(Cl)c(F)c1F ; *c1cc(F)c(Cl)cc1F ; *c1ccc(Cl)cc1C(F)(F)F]

R⁶ is H or methyl; and
R⁷ is Me.

In some embodiments, the compound is a compound of Formula IIIb

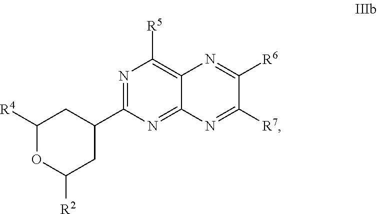

or a pharmaceutically acceptable salt thereof;
wherein
   R² is H or methyl,
   R⁴ is

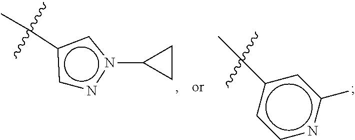

R⁵ is C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that:
when R⁴ is

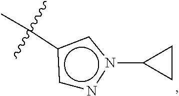

R⁵ is not

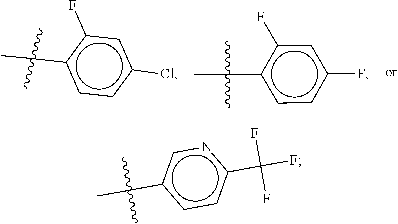

and
when R⁴ is

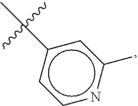

R⁵ is not

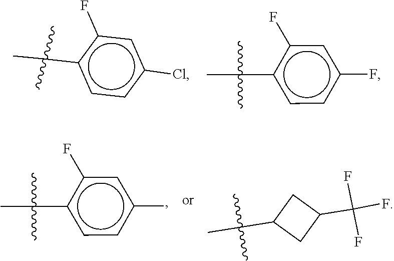

In some embodiments, the compound is a compound of Formula IIIb

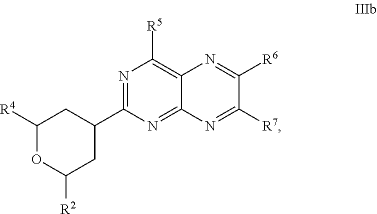

or a pharmaceutically acceptable salt thereof;
wherein
   R² is H or methyl;
   R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
   R⁵ is

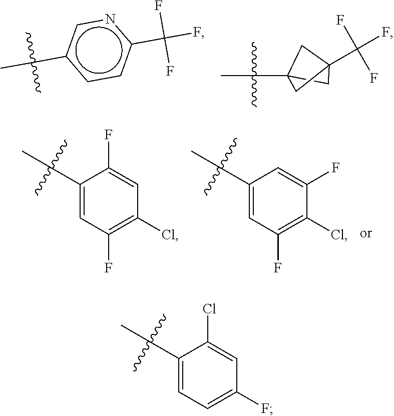

R⁶ is H or methyl; and
R⁷ is methyl;
provided that when R⁴ is

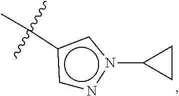

R⁵ is not

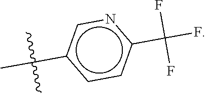

In some embodiments, the compound is a compound of Formula IIIb

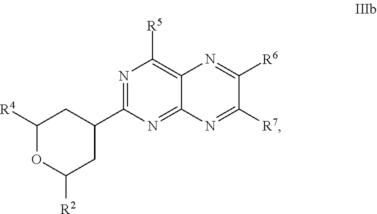

or a pharmaceutically acceptable salt thereof;
wherein
   R² is H or methyl;
   R⁴ is

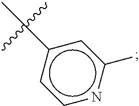

R⁵ is

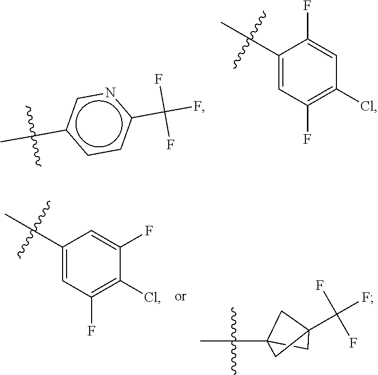

R⁶ is H or methyl; and
R⁷ is methyl.

In some embodiments, the compound is a compound of Formula IIIb

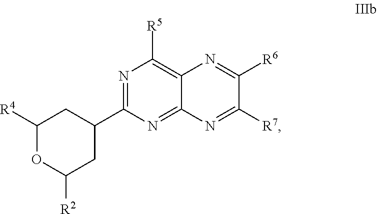

or a pharmaceutically acceptable salt thereof;
wherein
   R⁴ is

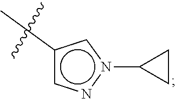

R⁵ is

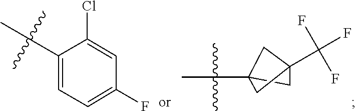 [SMILES: *c1ccc(F)cc1Cl ; *C12CC(C(F)(F)F)(C1)C2]

R⁶ is H or methyl; and
R⁷ is Me.

In some embodiments, the compound is a compound of Formula Va

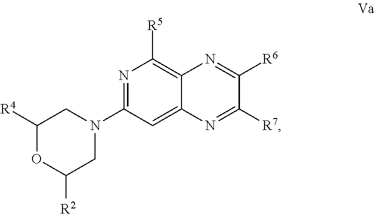

or a pharmaceutically acceptable salt thereof,
wherein
   R² is H or methyl;
   R⁴ is

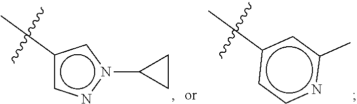

R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that:
when R⁶ is Me and R² is H, R⁵ is not

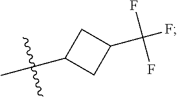 [SMILES: *C1CC(C(F)(F)F)C1]

and
when both R² and R⁶ are H, R⁵ is not

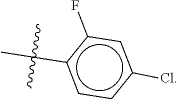

In some embodiments, the compound is a compound of Formula Vb

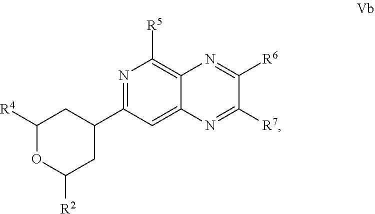

or a pharmaceutically acceptable salt thereof;
wherein
   R² is H or methyl;
   R⁴ is

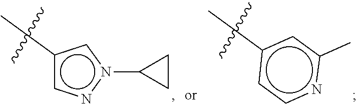

R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that when R² is H, R⁵ is not

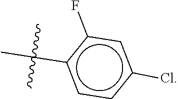

In some embodiments, the compound is a compound of Formula Va or Vb

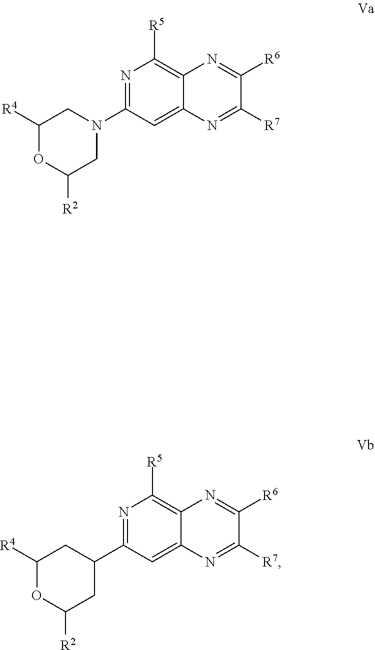

or a pharmaceutically acceptable salt thereof;
wherein
   R² is H or methyl;
   R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
   R⁵ is

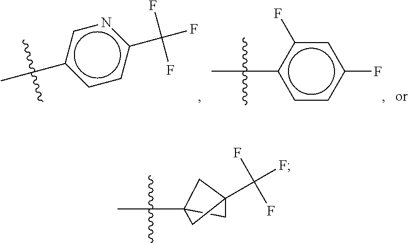

R⁶ is H or methyl; and
R⁷ is methyl.

In some embodiments, the compound is a compound of Formula Va or Vb

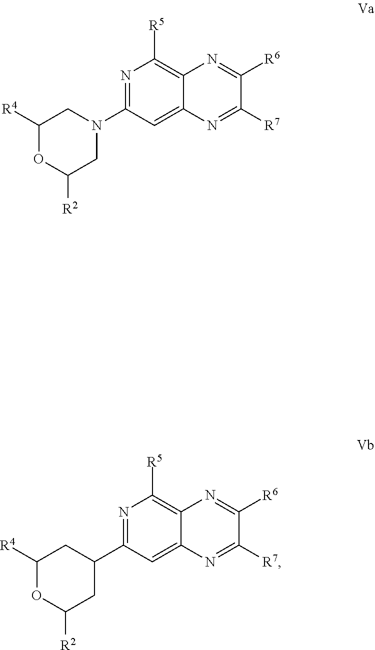

or a pharmaceutically acceptable salt thereof,
wherein
   R² is H or methyl;
   R⁴ is

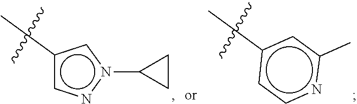

R⁵ is

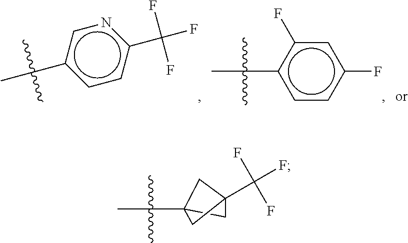

R⁶ is H or methyl; and
R⁷ is methyl.

In some embodiments, the compound is a compound of Formula Va or Vb

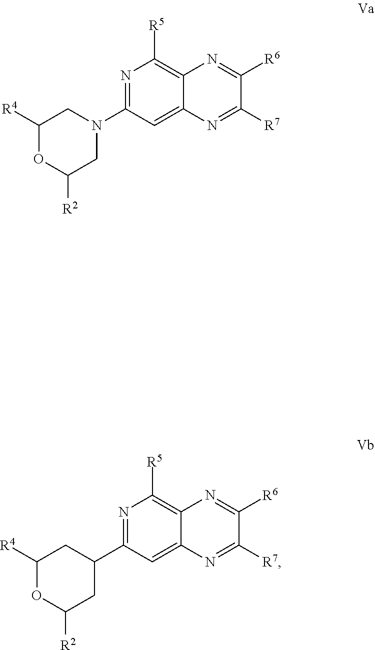

or a pharmaceutically acceptable salt thereof,
wherein
   R² is methyl;
   R⁴ is

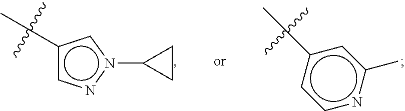

R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl), wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl.

In some embodiments, the compound is a compound of Formula Vb

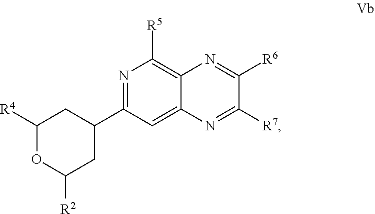

or a pharmaceutically acceptable salt thereof;
wherein
   R² is H or methyl;
   R⁴ is

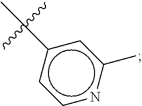

R⁵ is

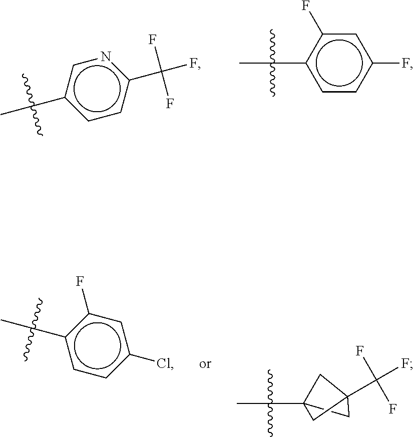

R⁶ is H or methyl; and
R⁷ is methyl;
provided that when R² is H, R⁵ is not

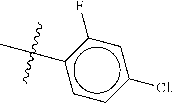

In some embodiments, the compound is a compound of Formula VIIIa

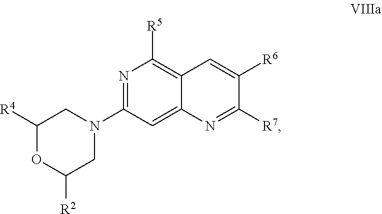

or a pharmaceutically acceptable salt thereof;
wherein
   R² is H or methyl;
   R⁴ is

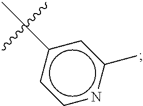

R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
   wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
   wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is Me
provided that R⁵ is not

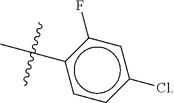

In some embodiments, the compound is a compound of Formula VIIIa

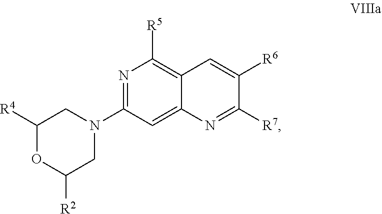

or a pharmaceutically acceptable salt thereof;
wherein
   R² is H or methyl;
   R⁴ is

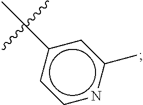

R⁵ is

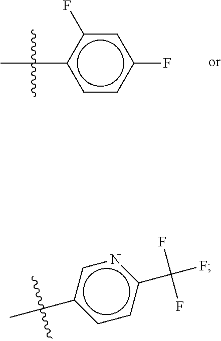

R⁶ is H or methyl; and
R⁷ is methyl.

In some embodiments, the compound is a compound of Formula VIIIb

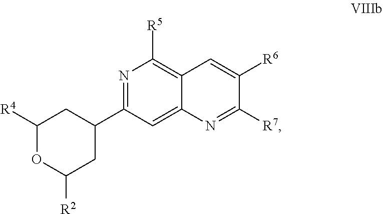

or a pharmaceutically acceptable salt thereof;
wherein
   R² is H or methyl;
   R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
   R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
      wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
      wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
   R⁶ is H or methyl; and
   R⁷ is methyl.

In some embodiments, the compound is a compound of Formula IVb

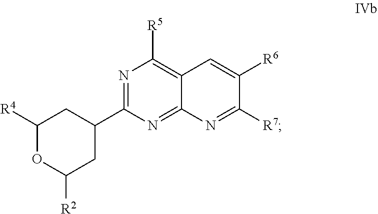

wherein
   R² is H or methyl;
   R⁴ is

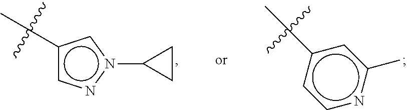

R⁵ is C₃₋₆cycloalkyl, C₅₋₈spiroalkyl, C₅₋₈stricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
   R⁶ is H or methyl; and
   R⁷ is Me;
   provided that when R² is H, R⁵ is not

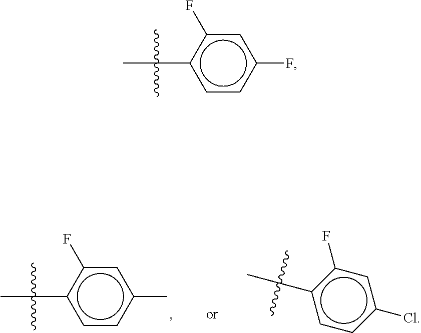

Exemplary compounds of the invention are set forth in Table A, below. In some embodiments, the compound is a compound set forth in Table A, or a pharmaceutically acceptable salt thereof.

TABLE A
Exemplary Compounds

| I-# | Structure |
|---|---|
| I-1 | 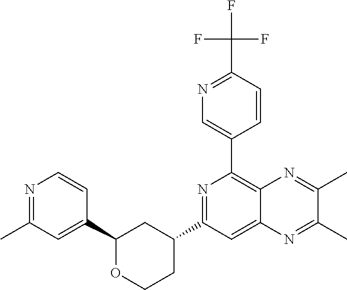 |
| I-2 | 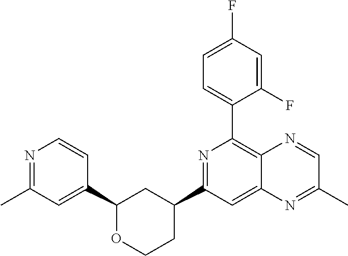 |
| I-3 | 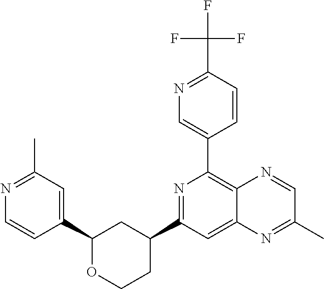 [SMILES: Cc1cc([C@H]2C[C@@H](c3cc4nc(C)cnc4c(-c4ccc(C(F)(F)F)nc4)n3)CCO2)ccn1] |
| I-4 | 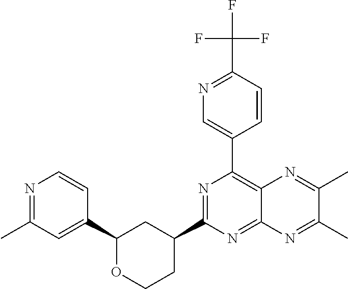 |
| I-5 | 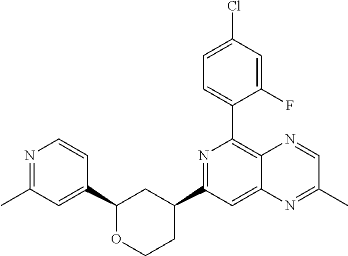 |
| I-6 | 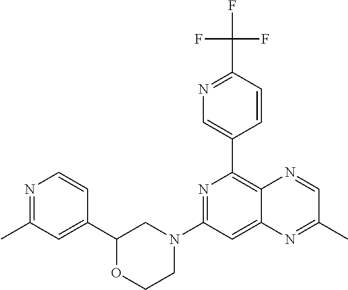 |
| I-7 | 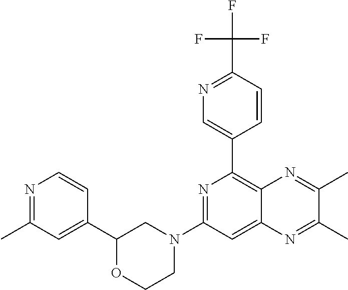 |
| I-8 | 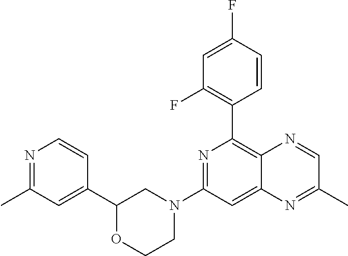 |
| I-9 | 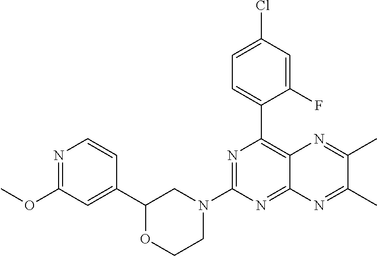 |
| I-10 | 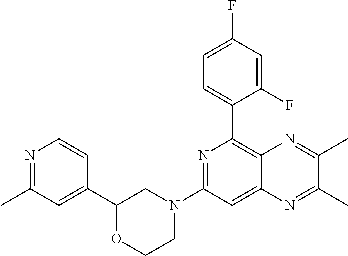 |
| I-11 | 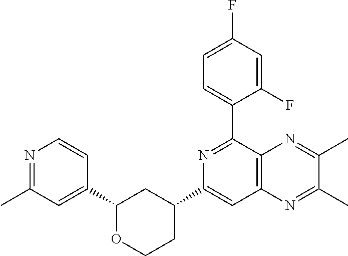 |
| I-12 | 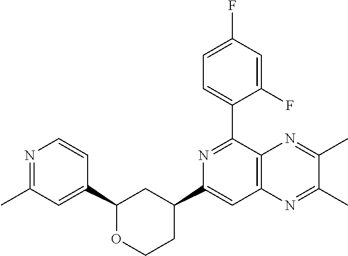 |
| I-13 | 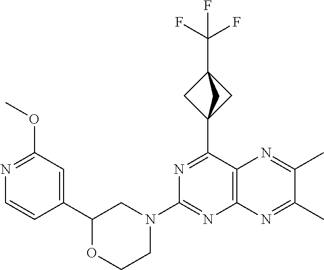 [SMILES: COc1cc(C2CN(c3nc4nc(C)c(C)nc4c([C@]45C[C@](C(F)(F)F)(C4)C5)n3)CCO2)ccn1] |
| I-14 | 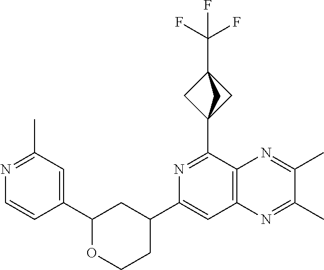 [SMILES: Cc1cc(C2CC(c3cc4nc(C)c(C)nc4c([C@]45C[C@](C(F)(F)F)(C4)C5)n3)CCO2)ccn1] |
| I-15 | 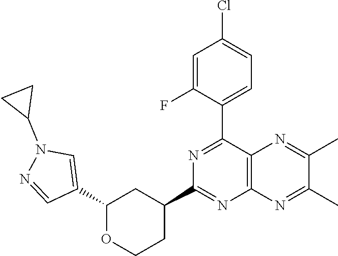 [SMILES: Cc1nc2nc([C@H]3CCO[C@H](c4cnn(C5CC5)c4)C3)nc(-c3ccc(Cl)cc3F)c2nc1C] |
| I-16 | 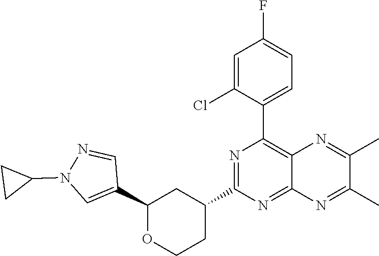 [SMILES: Cc1nc2nc([C@@H]3CCO[C@@H](c4cnn(C5CC5)c4)C3)nc(-c3ccc(F)cc3Cl)c2nc1C] |
| I-17 | 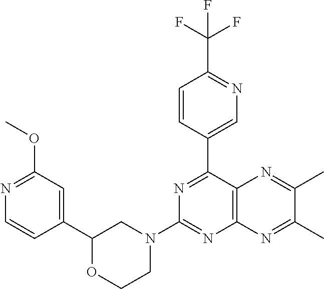 [SMILES: COc1cc(C2CN(c3nc(-c4ccc(C(F)(F)F)nc4)c4nc(C)c(C)nc4n3)CCO2)ccn1] |
| I-18 | 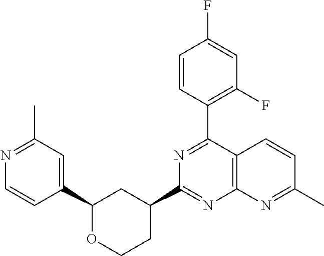 [SMILES: Cc1cc([C@H]2C[C@@H](c3nc(-c4ccc(F)cc4F)c4ccc(C)nc4n3)CCO2)ccn1] |
| I-19 | 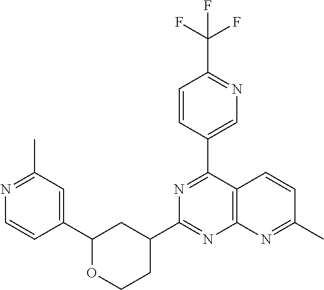 [SMILES: Cc1cc(C2CC(c3nc(-c4ccc(C(F)(F)F)nc4)c4ccc(C)nc4n3)CCO2)ccn1] |
| I-20 | 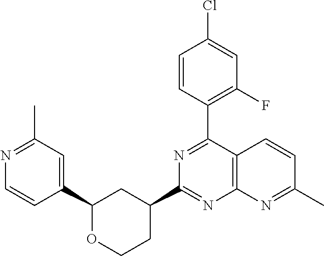 [SMILES: Cc1cc([C@H]2C[C@@H](c3nc(-c4ccc(Cl)cc4F)c4ccc(C)nc4n3)CCO2)ccn1] |
| I-21 | 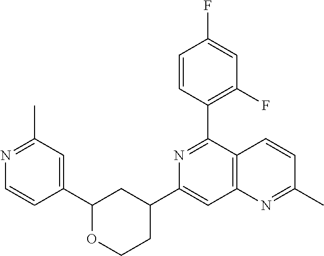 [SMILES: Cc1cc(C2CC(c3cc4nc(C)ccc4c(-c4ccc(F)cc4F)n3)CCO2)ccn1] |
| I-22 | 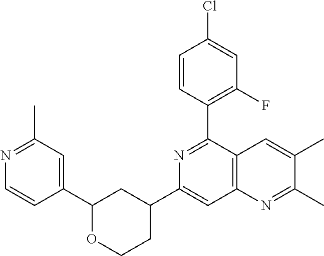 [SMILES: Cc1cc(C2CC(c3cc4nc(C)c(C)cc4c(-c4ccc(Cl)cc4F)n3)CCO2)ccn1] |
| I-23 | 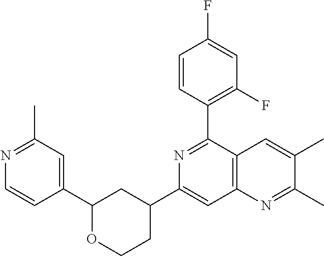 [SMILES: Cc1cc(C2CC(c3cc4nc(C)c(C)cc4c(-c4ccc(F)cc4F)n3)CCO2)ccn1] |
| I-24 | 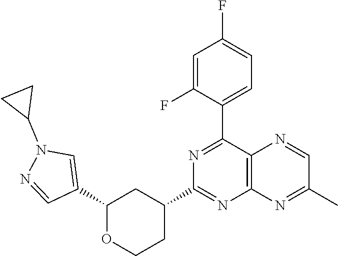 [SMILES: Cc1cnc2c(-c3ccc(F)cc3F)nc([C@@H]3CCO[C@H](c4cnn(C5CC5)c4)C3)nc2n1] |
| I-25 | 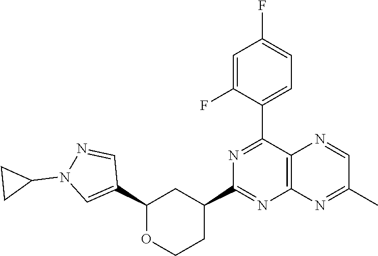 [SMILES: Cc1cnc2c(-c3ccc(F)cc3F)nc([C@H]3CCO[C@@H](c4cnn(C5CC5)c4)C3)nc2n1] |
| I-26 | 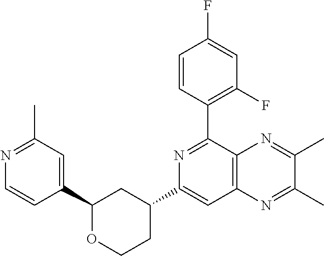 [SMILES: Cc1cc([C@H]2C[C@H](c3cc4nc(C)c(C)nc4c(-c4ccc(F)cc4F)n3)CCO2)ccn1] |
| I-27 | 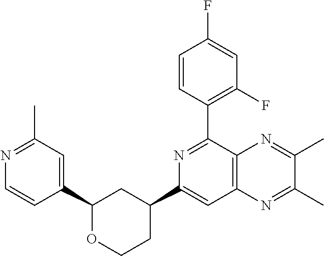 [SMILES: Cc1cc([C@H]2C[C@@H](c3cc4nc(C)c(C)nc4c(-c4ccc(F)cc4F)n3)CCO2)ccn1] |
| I-28 | 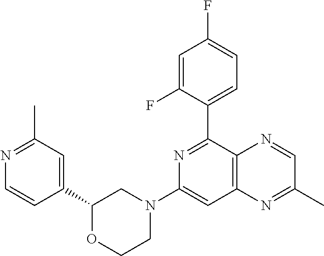 [SMILES: Cc1cc([C@@H]2CN(c3cc4nc(C)cnc4c(-c4ccc(F)cc4F)n3)CCO2)ccn1] |
| I-29 | 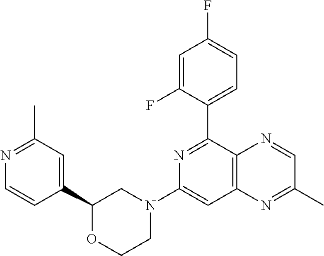 [SMILES: Cc1cc([C@H]2CN(c3cc4nc(C)cnc4c(-c4ccc(F)cc4F)n3)CCO2)ccn1] |
| I-30 | 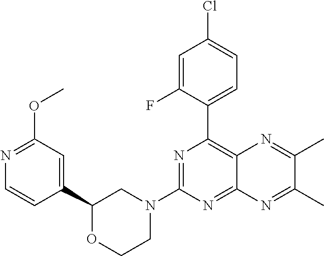 [SMILES: COc1cc([C@H]2CN(c3nc(-c4ccc(Cl)cc4F)c4nc(C)c(C)nc4n3)CCO2)ccn1] |
| I-31 | 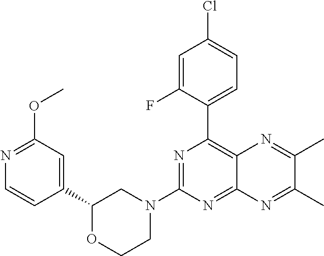 [SMILES: COc1cc([C@@H]2CN(c3nc(-c4ccc(Cl)cc4F)c4nc(C)c(C)nc4n3)CCO2)ccn1] |
| I-32 | 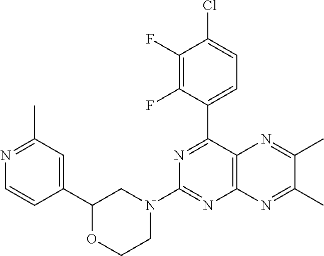 [SMILES: Cc1cc(C2CN(c3nc(-c4ccc(Cl)c(F)c4F)c4nc(C)c(C)nc4n3)CCO2)ccn1] |
| I-33 | 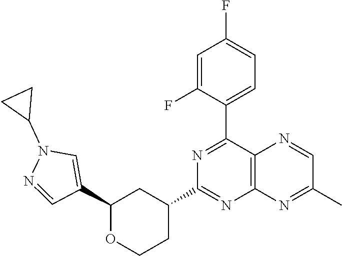 [SMILES: Cc1cnc2c(-c3ccc(F)cc3F)nc([C@@H]3CCO[C@@H](c4cnn(C5CC5)c4)C3)nc2n1] |
| I-34 | 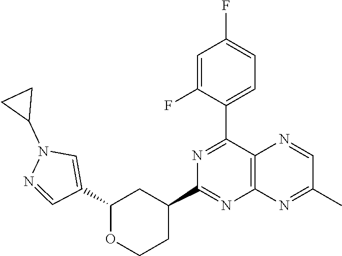 [SMILES: Cc1cnc2c(-c3ccc(F)cc3F)nc([C@H]3CCO[C@H](c4cnn(C5CC5)c4)C3)nc2n1] |
| I-35 | 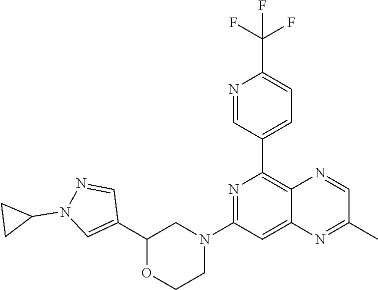 [SMILES: Cc1cnc2c(-c3ccc(C(F)(F)F)nc3)nc(N3CCOC(c4cnn(C5CC5)c4)C3)cc2n1] |
| I-36 | 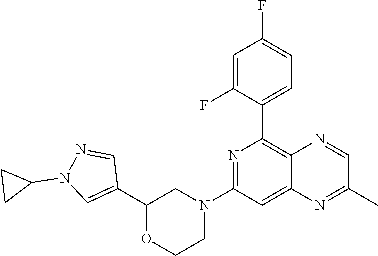 [SMILES: Cc1cnc2c(-c3ccc(F)cc3F)nc(N3CCOC(c4cnn(C5CC5)c4)C3)cc2n1] |
| I-37 | 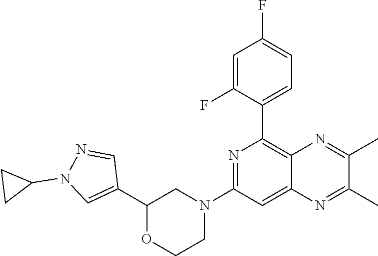 [SMILES: Cc1nc2cc(N3CCOC(c4cnn(C5CC5)c4)C3)nc(-c3ccc(F)cc3F)c2nc1C] |
| I-38 | 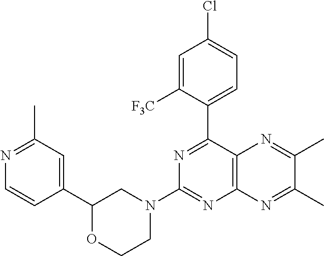 [SMILES: Cc1cc(C2CN(c3nc(-c4ccc(Cl)cc4C(F)(F)F)c4nc(C)c(C)nc4n3)CCO2)ccn1] |
| I-39 | 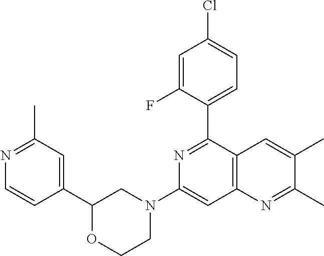 [SMILES: Cc1cc(C2CN(c3cc4nc(C)c(C)cc4c(-c4ccc(Cl)cc4F)n3)CCO2)ccn1] |
| I-40 | 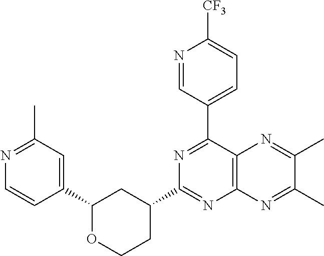 [SMILES: Cc1cc([C@@H]2C[C@H](c3nc(-c4ccc(C(F)(F)F)nc4)c4nc(C)c(C)nc4n3)CCO2)ccn1] |
| I-41 | 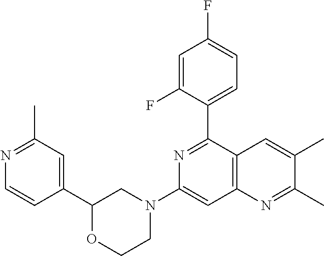 [SMILES: Cc1cc(C2CN(c3cc4nc(C)c(C)cc4c(-c4ccc(F)cc4F)n3)CCO2)ccn1] |
| I-42 | 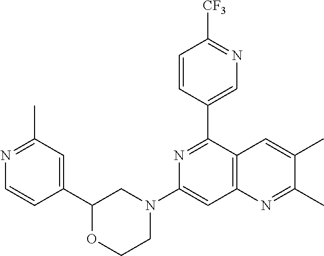 [SMILES: Cc1cc(C2CN(c3cc4nc(C)c(C)cc4c(-c4ccc(C(F)(F)F)nc4)n3)CCO2)ccn1] |
| I-43 | 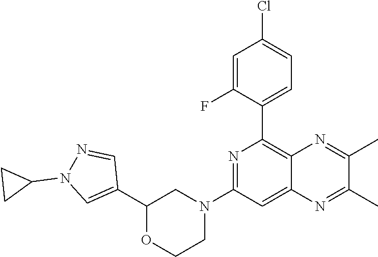 [SMILES: Cc1nc2cc(N3CCOC(c4cnn(C5CC5)c4)C3)nc(-c3ccc(Cl)cc3F)c2nc1C] |
| I-44 | 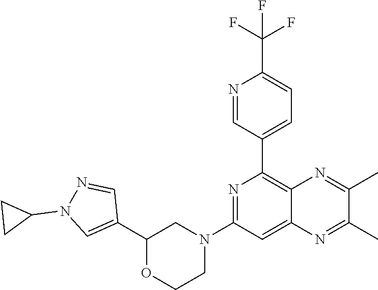 [SMILES: Cc1nc2cc(N3CCOC(c4cnn(C5CC5)c4)C3)nc(-c3ccc(C(F)(F)F)nc3)c2nc1C] |
| I-45 | 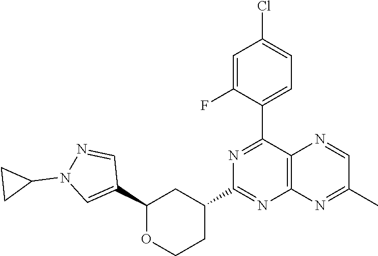 [SMILES: Cc1cnc2c(-c3ccc(Cl)cc3F)nc([C@@H]3CCO[C@@H](c4cnn(C5CC5)c4)C3)nc2n1] |
| I-46 | 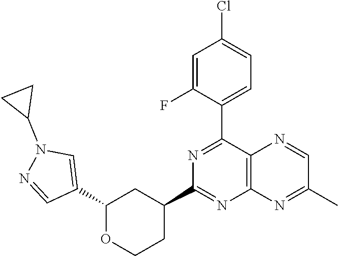 [SMILES: Cc1cnc2c(-c3ccc(Cl)cc3F)nc([C@H]3CCO[C@H](c4cnn(C5CC5)c4)C3)nc2n1] |
| I-47 | 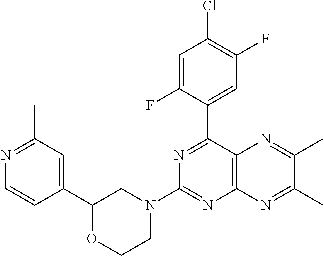 [SMILES: Cc1cc(C2CN(c3nc(-c4cc(F)c(Cl)cc4F)c4nc(C)c(C)nc4n3)CCO2)ccn1] |
| I-48 | 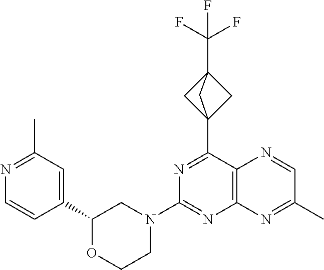 [SMILES: Cc1cc([C@@H]2CN(c3nc(C45CC(C(F)(F)F)(C4)C5)c4ncc(C)nc4n3)CCO2)ccn1] |
| I-49 | 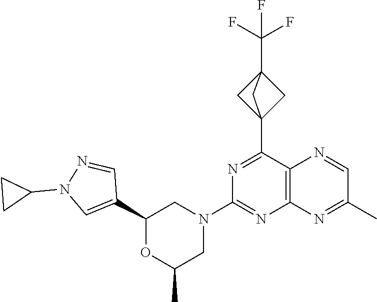 [SMILES: Cc1cnc2c(C34CC(C(F)(F)F)(C3)C4)nc(N3C[C@@H](C)O[C@@H](c4cnn(C5CC5)c4)C3)nc2n1] |
| I-50 | 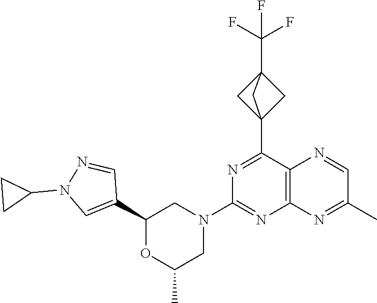 [SMILES: Cc1cnc2c(C34CC(C(F)(F)F)(C3)C4)nc(N3C[C@H](C)O[C@@H](c4cnn(C5CC5)c4)C3)nc2n1] |
| I-51 | 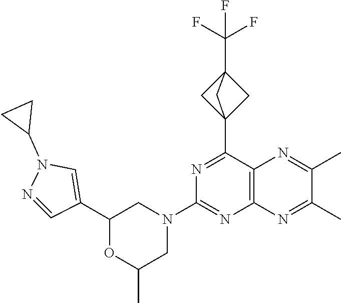 [SMILES: Cc1nc2nc(N3CC(C)OC(c4cnn(C5CC5)c4)C3)nc(C34CC(C(F)(F)F)(C3)C4)c2nc1C] |
| I-52 | 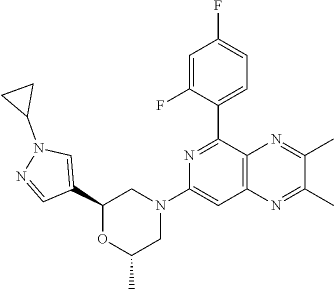 [SMILES: Cc1nc2cc(N3C[C@H](C)O[C@@H](c4cnn(C5CC5)c4)C3)nc(-c3ccc(F)cc3F)c2nc1C] |
| I-53 | 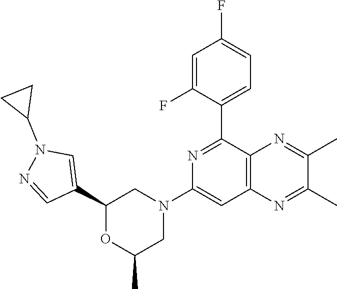 [SMILES: Cc1nc2cc(N3C[C@@H](C)O[C@@H](c4cnn(C5CC5)c4)C3)nc(-c3ccc(F)cc3F)c2nc1C] |
| I-54 | 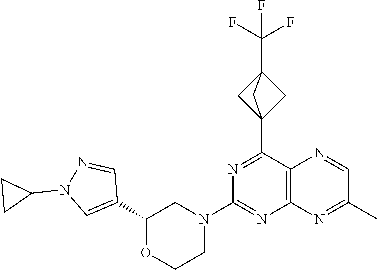 [SMILES: Cc1cnc2c(C34CC(C(F)(F)F)(C3)C4)nc(N3CCO[C@H](c4cnn(C5CC5)c4)C3)nc2n1] |
| I-55 | 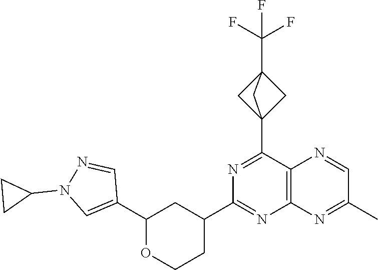 [SMILES: Cc1cnc2c(C34CC(C(F)(F)F)(C3)C4)nc(C3CCOC(c4cnn(C5CC5)c4)C3)nc2n1] |
| I-56 | 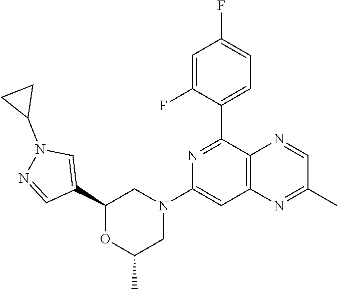 [SMILES: Cc1cnc2c(-c3ccc(F)cc3F)nc(N3C[C@H](C)O[C@@H](c4cnn(C5CC5)c4)C3)cc2n1] |
| I-57 | 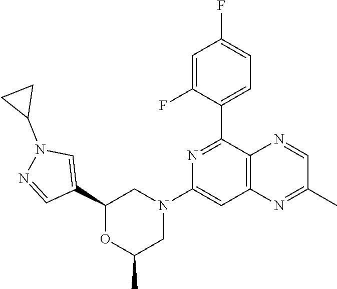 [SMILES: Cc1cnc2c(-c3ccc(F)cc3F)nc(N3C[C@@H](C)O[C@@H](c4cnn(C5CC5)c4)C3)cc2n1] |
| I-58 | 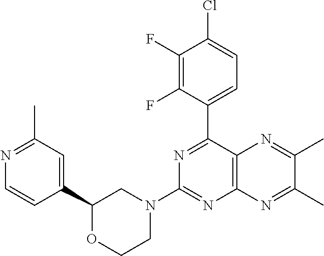 [SMILES: Cc1cc([C@H]2CN(c3nc(-c4ccc(Cl)c(F)c4F)c4nc(C)c(C)nc4n3)CCO2)ccn1] |
| I-59 | 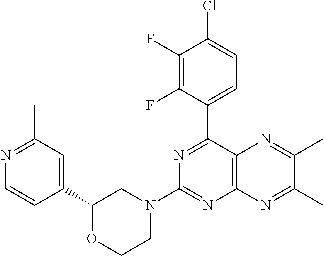 [SMILES: Cc1cc([C@@H]2CN(c3nc(-c4ccc(Cl)c(F)c4F)c4nc(C)c(C)nc4n3)CCO2)ccn1] |
| I-60 | 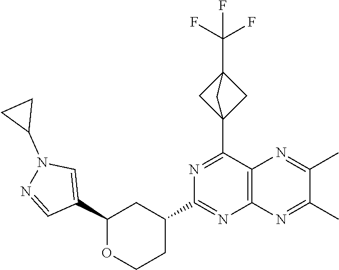 [SMILES: Cc1nc2nc([C@@H]3CCO[C@@H](c4cnn(C5CC5)c4)C3)nc(C34CC(C(F)(F)F)(C3)C4)c2nc1C] |
| I-61 | 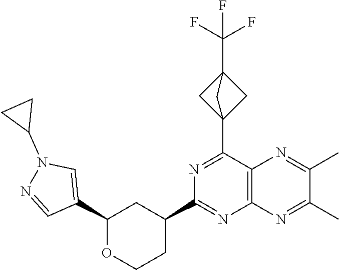 [SMILES: Cc1nc2nc([C@H]3CCO[C@@H](c4cnn(C5CC5)c4)C3)nc(C34CC(C(F)(F)F)(C3)C4)c2nc1C] |
| I-62 | 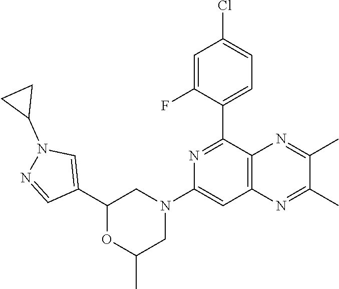 [SMILES: Cc1nc2cc(N3CC(C)OC(c4cnn(C5CC5)c4)C3)nc(-c3ccc(Cl)cc3F)c2nc1C] |
| I-63 | 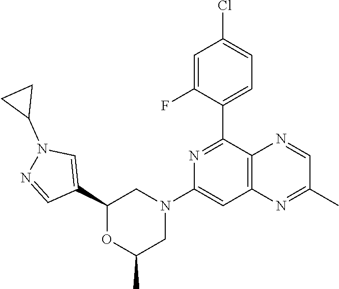 [SMILES: Cc1cnc2c(-c3ccc(Cl)cc3F)nc(N3C[C@@H](C)O[C@@H](c4cnn(C5CC5)c4)C3)cc2n1] |
| I-64 | 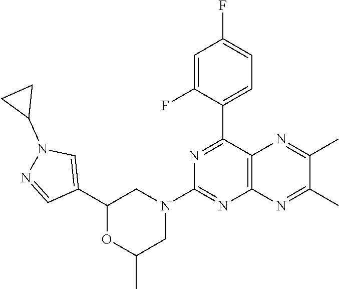 [SMILES: Cc1nc2nc(N3CC(C)OC(c4cnn(C5CC5)c4)C3)nc(-c3ccc(F)cc3F)c2nc1C] |
| I-65 | 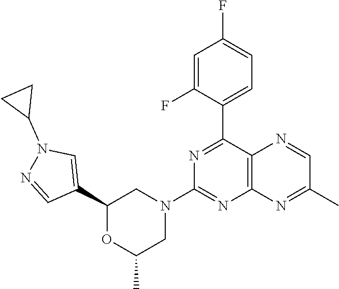 [SMILES: Cc1cnc2c(-c3ccc(F)cc3F)nc(N3C[C@H](C)O[C@@H](c4cnn(C5CC5)c4)C3)nc2n1] |
| I-66 | 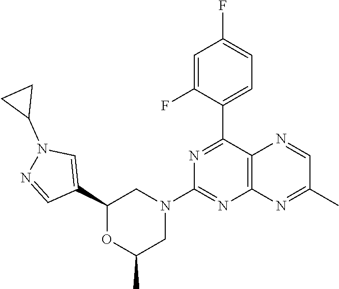 [SMILES: Cc1cnc2c(-c3ccc(F)cc3F)nc(N3C[C@@H](C)O[C@@H](c4cnn(C5CC5)c4)C3)nc2n1] |
| I-67 | 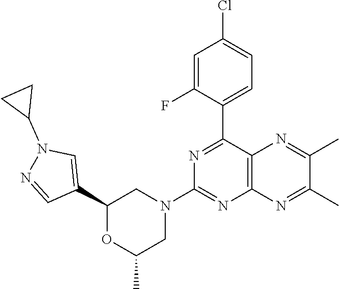 [SMILES: Cc1nc2nc(N3C[C@H](C)O[C@@H](c4cnn(C5CC5)c4)C3)nc(-c3ccc(Cl)cc3F)c2nc1C] |
| I-68 | 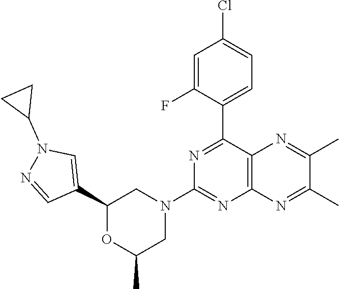 [SMILES: Cc1nc2nc(N3C[C@@H](C)O[C@@H](c4cnn(C5CC5)c4)C3)nc(-c3ccc(Cl)cc3F)c2nc1C] |
| I-69 | 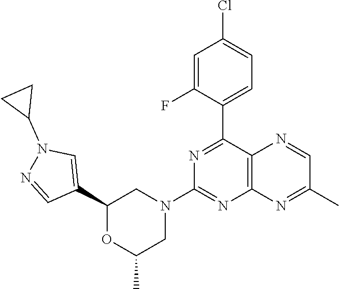 [SMILES: Cc1cnc2c(-c3ccc(Cl)cc3F)nc(N3C[C@H](C)O[C@@H](c4cnn(C5CC5)c4)C3)nc2n1] |
| I-70 | 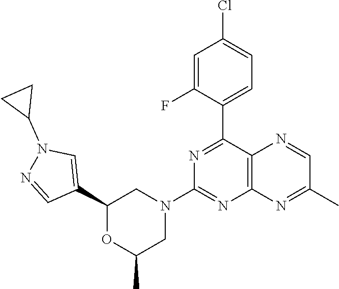 [SMILES: Cc1cnc2c(-c3ccc(Cl)cc3F)nc(N3C[C@@H](C)O[C@@H](c4cnn(C5CC5)c4)C3)nc2n1] |
| I-71 | 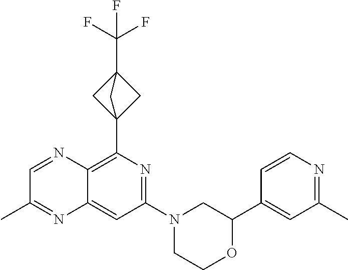 |
| I-72 | 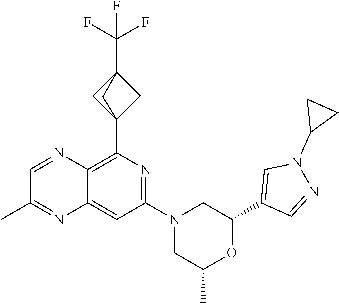 [SMILES: Cc1cnc2c(C34CC(C(F)(F)F)(C3)C4)nc(N3C[C@@H](C)O[C@@H](c4cnn(C5CC5)c4)C3)cc2n1] |
| I-73 | 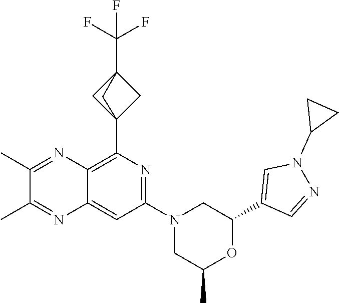 [SMILES: Cc1nc2cc(N3C[C@H](C)O[C@@H](c4cnn(C5CC5)c4)C3)nc(C34CC(C(F)(F)F)(C3)C4)c2nc1C] |
| I-74 | 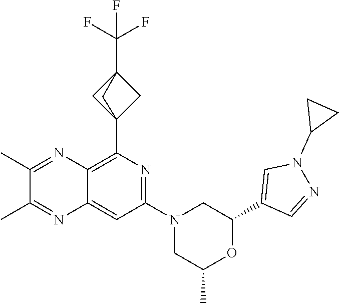 [SMILES: Cc1nc2cc(N3C[C@@H](C)O[C@@H](c4cnn(C5CC5)c4)C3)nc(C34CC(C(F)(F)F)(C3)C4)c2nc1C] |
| I-75 | 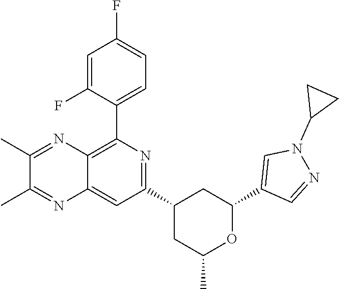 [SMILES: Cc1nc2cc([C@H]3C[C@@H](C)O[C@@H](c4cnn(C5CC5)c4)C3)nc(-c3ccc(F)cc3F)c2nc1C] |
| I-76 | 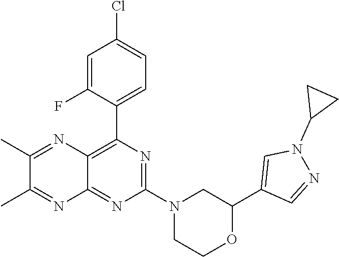 [SMILES: Cc1nc2nc(N3CCOC(c4cnn(C5CC5)c4)C3)nc(-c3ccc(Cl)cc3F)c2nc1C] |
| I-77 | 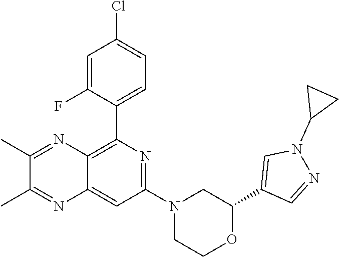 [SMILES: Cc1nc2cc(N3CCO[C@@H](c4cnn(C5CC5)c4)C3)nc(-c3ccc(Cl)cc3F)c2nc1C] |
| I-78 | 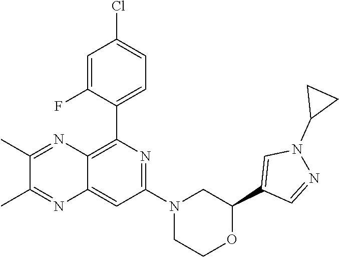 [SMILES: Cc1nc2cc(N3CCO[C@H](c4cnn(C5CC5)c4)C3)nc(-c3ccc(Cl)cc3F)c2nc1C] |
| I-79 | 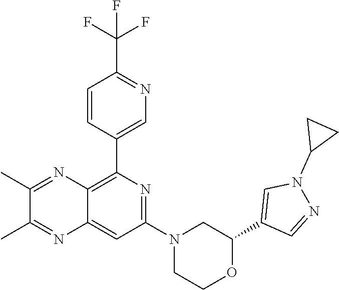 [SMILES: Cc1nc2cc(N3CCO[C@@H](c4cnn(C5CC5)c4)C3)nc(-c3ccc(C(F)(F)F)nc3)c2nc1C] |
| I-80 | 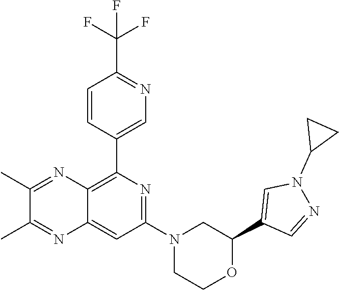 [SMILES: Cc1nc2cc(N3CCO[C@H](c4cnn(C5CC5)c4)C3)nc(-c3ccc(C(F)(F)F)nc3)c2nc1C] |
| I-81 | 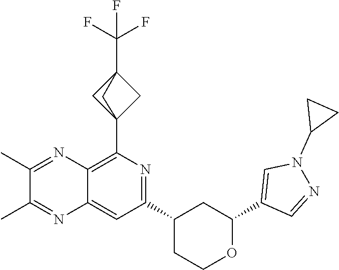 [SMILES: Cc1nc2cc([C@H]3CCO[C@@H](c4cnn(C5CC5)c4)C3)nc(C34CC(C(F)(F)F)(C3)C4)c2nc1C] |
| I-82 | 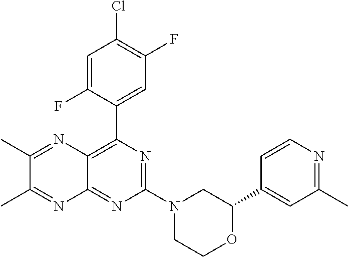 |
| I-83 | 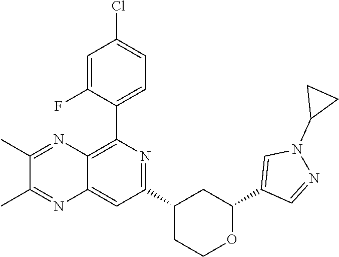 [SMILES: Cc1nc2cc([C@H]3CCO[C@@H](c4cnn(C5CC5)c4)C3)nc(-c3ccc(Cl)cc3F)c2nc1C] |
| I-84 | 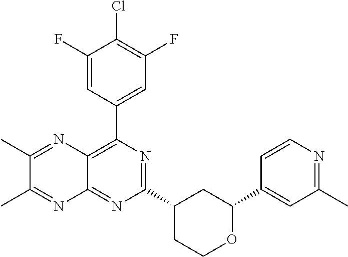 |
| I-85 | 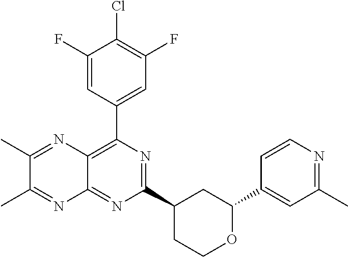 |
| I-86 | 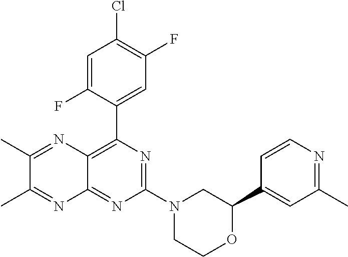 |
| I-87 | 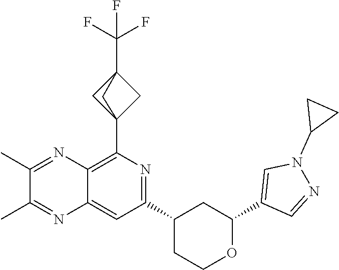 [SMILES: Cc1nc2cc([C@H]3CCO[C@@H](c4cnn(C5CC5)c4)C3)nc(C34CC(C(F)(F)F)(C3)C4)c2nc1C] |
| I-88 | 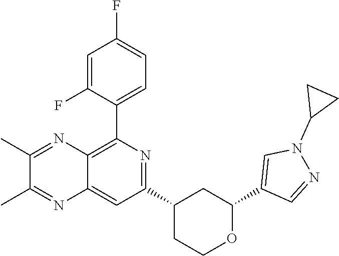 [SMILES: Cc1nc2cc([C@H]3CCO[C@@H](c4cnn(C5CC5)c4)C3)nc(-c3ccc(F)cc3F)c2nc1C] |
| I-89 | 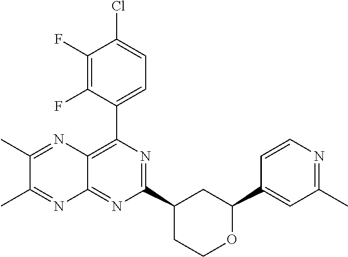 |
| I-90 | 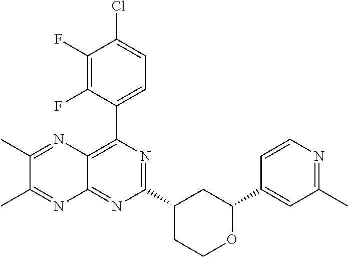 |
| I-91 | 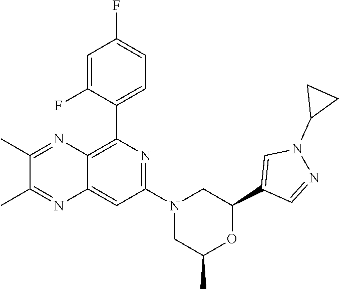 [SMILES: Cc1nc2cc(N3C[C@@H](c4cnn(C5CC5)c4)O[C@@H](C)C3)nc(-c3ccc(F)cc3F)c2nc1C] |
| I-92 | 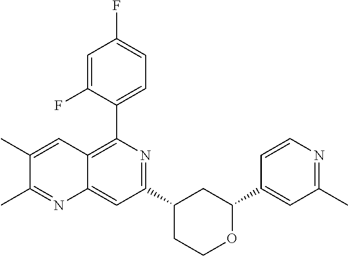 |
| I-93 | 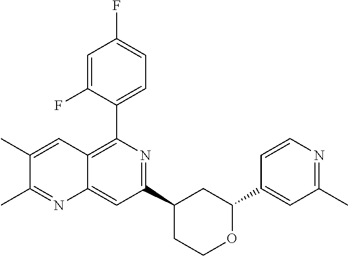 |
| I-94 | 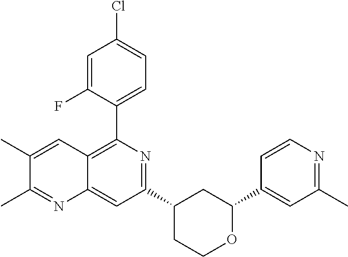 |
| I-95 | 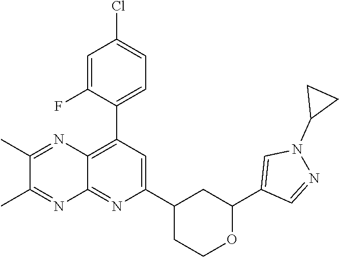 [SMILES: Cc1nc2nc(C3CCOC(c4cnn(C5CC5)c4)C3)cc(-c3ccc(Cl)cc3F)c2nc1C] |
| I-96 | 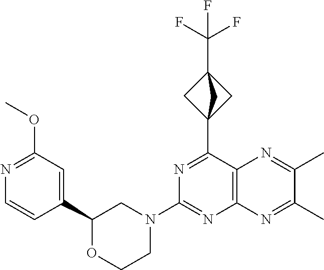 [SMILES: COc1cc([C@H]2CN(c3nc4nc(C)c(C)nc4c([C@]45C[C@](C(F)(F)F)(C4)C5)n3)CCO2)ccn1] |
| I-97 | 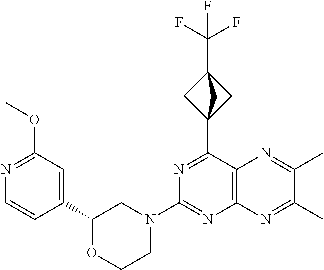 [SMILES: COc1cc([C@@H]2CN(c3nc4nc(C)c(C)nc4c([C@]45C[C@](C(F)(F)F)(C4)C5)n3)CCO2)ccn1] |
| I-98 | 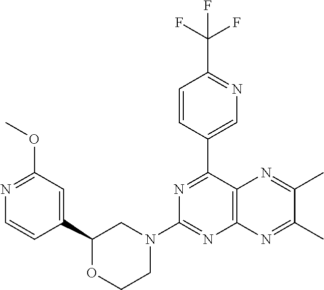 [SMILES: COc1cc([C@H]2CN(c3nc(-c4ccc(C(F)(F)F)nc4)c4nc(C)c(C)nc4n3)CCO2)ccn1] |
| I-99 | 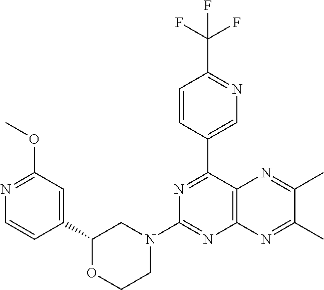 [SMILES: COc1cc([C@@H]2CN(c3nc(-c4ccc(C(F)(F)F)nc4)c4nc(C)c(C)nc4n3)CCO2)ccn1] |
| I-100 | 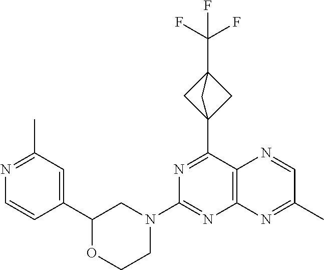 [SMILES: Cc1cc(C2CN(c3nc(C45CC(C(F)(F)F)(C4)C5)c4ncc(C)nc4n3)CCO2)ccn1] |
| I-101 | 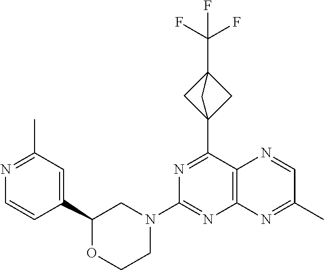 [SMILES: Cc1cc([C@H]2CN(c3nc(C45CC(C(F)(F)F)(C4)C5)c4ncc(C)nc4n3)CCO2)ccn1] |
| I-102 | 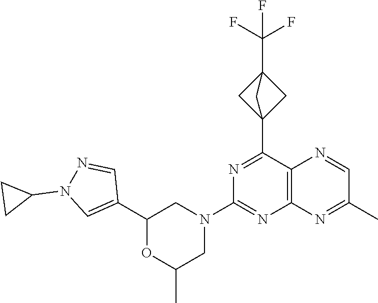 [SMILES: Cc1cnc2c(C34CC(C(F)(F)F)(C3)C4)nc(N3CC(C)OC(c4cnn(C5CC5)c4)C3)nc2n1] |
| I-103 | 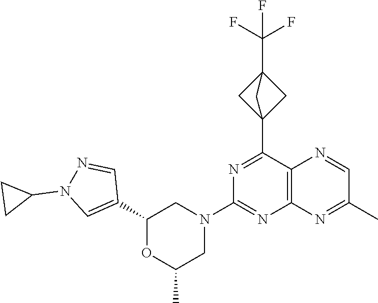 [SMILES: Cc1cnc2c(C34CC(C(F)(F)F)(C3)C4)nc(N3C[C@@H](c4cnn(C5CC5)c4)O[C@@H](C)C3)nc2n1] |
| I-104 | 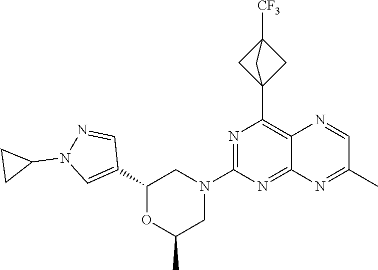 [SMILES: Cc1cnc2c(C34CC(C(F)(F)F)(C3)C4)nc(N3C[C@@H](C)O[C@H](c4cnn(C5CC5)c4)C3)nc2n1] |
| I-105 | 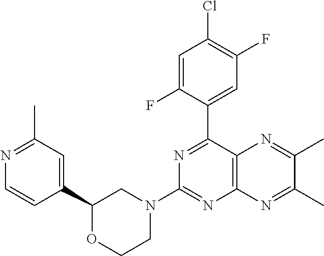 [SMILES: Cc1cc([C@H]2CN(c3nc(-c4cc(F)c(Cl)cc4F)c4nc(C)c(C)nc4n3)CCO2)ccn1] |
| I-106 | 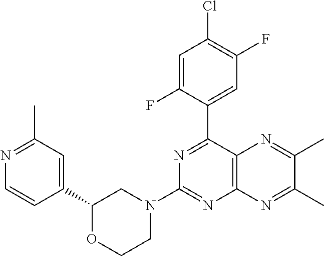 [SMILES: Cc1cc([C@@H]2CN(c3nc(-c4cc(F)c(Cl)cc4F)c4nc(C)c(C)nc4n3)CCO2)ccn1] |
| I-107 | 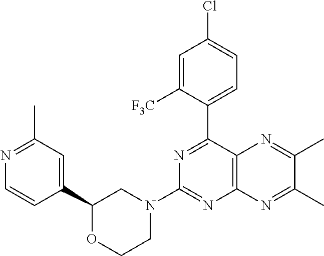 [SMILES: Cc1cc([C@H]2CN(c3nc(-c4ccc(Cl)cc4C(F)(F)F)c4nc(C)c(C)nc4n3)CCO2)ccn1] |
| I-108 | 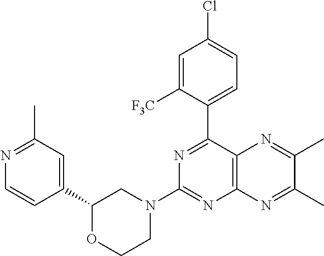 [SMILES: Cc1cc([C@@H]2CN(c3nc(-c4ccc(Cl)cc4C(F)(F)F)c4nc(C)c(C)nc4n3)CCO2)ccn1] |
| I-109 | 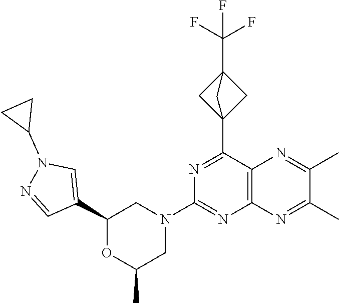 [SMILES: Cc1nc2nc(N3C[C@@H](C)O[C@@H](c4cnn(C5CC5)c4)C3)nc(C34CC(C(F)(F)F)(C3)C4)c2nc1C] |
| I-110 | 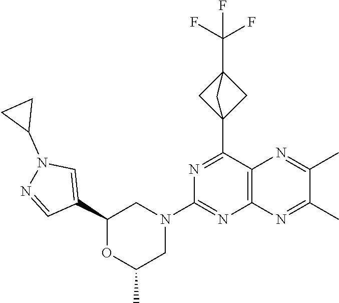 [SMILES: Cc1nc2nc(N3C[C@H](C)O[C@@H](c4cnn(C5CC5)c4)C3)nc(C34CC(C(F)(F)F)(C3)C4)c2nc1C] |
| I-111 | 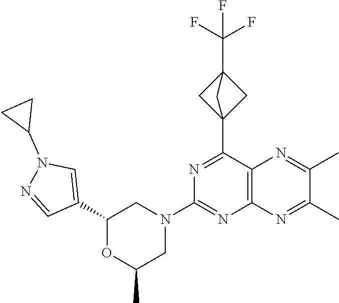 [SMILES: Cc1nc2nc(N3C[C@@H](C)O[C@H](c4cnn(C5CC5)c4)C3)nc(C34CC(C(F)(F)F)(C3)C4)c2nc1C] |
| I-112 | 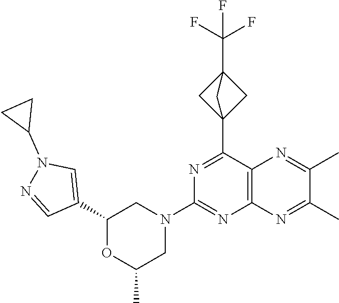 [SMILES: Cc1nc2nc(N3C[C@@H](c4cnn(C5CC5)c4)O[C@@H](C)C3)nc(C34CC(C(F)(F)F)(C3)C4)c2nc1C] |
| I-113 | 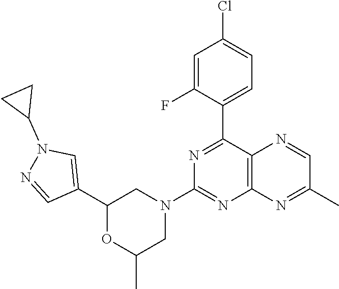 [SMILES: Cc1cnc2c(-c3ccc(Cl)cc3F)nc(N3CC(C)OC(c4cnn(C5CC5)c4)C3)nc2n1] |
| I-114 | 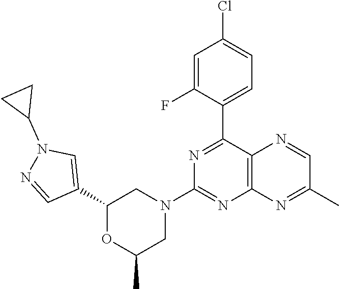 [SMILES: Cc1cnc2c(-c3ccc(Cl)cc3F)nc(N3C[C@@H](C)O[C@H](c4cnn(C5CC5)c4)C3)nc2n1] |
| I-115 | 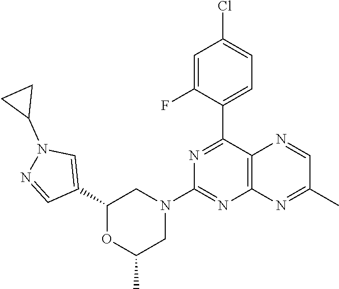 [SMILES: Cc1cnc2c(-c3ccc(Cl)cc3F)nc(N3C[C@@H](c4cnn(C5CC5)c4)O[C@@H](C)C3)nc2n1] |
| I-116 | 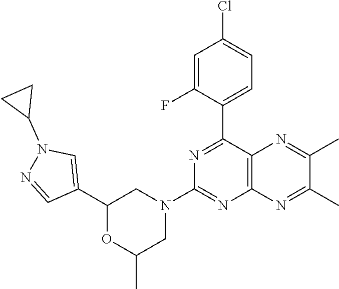 [SMILES: Cc1nc2nc(N3CC(C)OC(c4cnn(C5CC5)c4)C3)nc(-c3ccc(Cl)cc3F)c2nc1C] |
| I-117 | 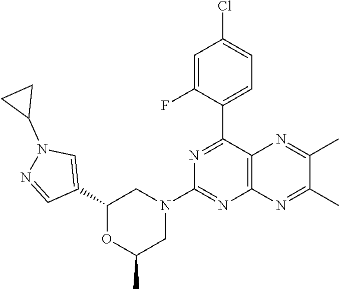 [SMILES: Cc1nc2nc(N3C[C@@H](C)O[C@H](c4cnn(C5CC5)c4)C3)nc(-c3ccc(Cl)cc3F)c2nc1C] |
| I-118 | 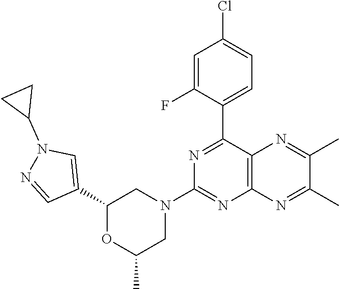 [SMILES: Cc1nc2nc(N3C[C@@H](c4cnn(C5CC5)c4)O[C@@H](C)C3)nc(-c3ccc(Cl)cc3F)c2nc1C] |
| I-119 | 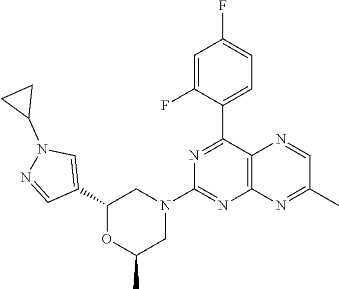 [SMILES: Cc1cnc2c(-c3ccc(F)cc3F)nc(N3C[C@@H](C)O[C@H](c4cnn(C5CC5)c4)C3)nc2n1] |
| I-120 | 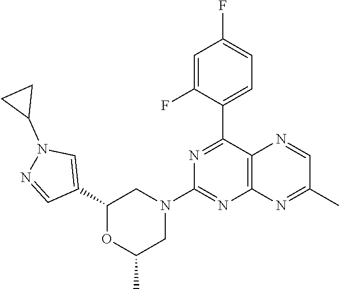 [SMILES: Cc1cnc2c(-c3ccc(F)cc3F)nc(N3C[C@@H](c4cnn(C5CC5)c4)O[C@@H](C)C3)nc2n1] |
| I-121 | 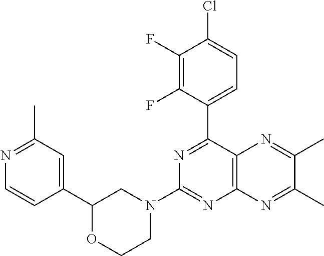 [SMILES: Cc1cc(C2CN(c3nc(-c4ccc(Cl)c(F)c4F)c4nc(C)c(C)nc4n3)CCO2)ccn1] |
| I-122 | 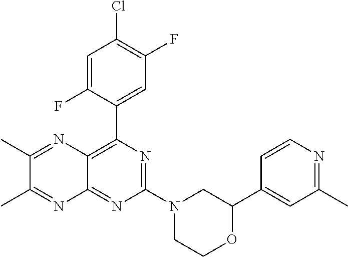 |
| I-123 | 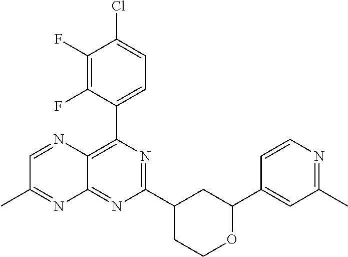 |
| I-124 | 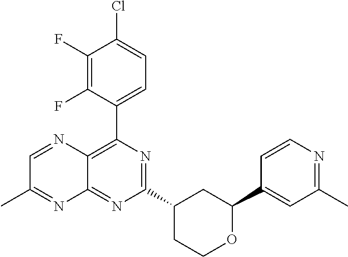 |
| I-125 | 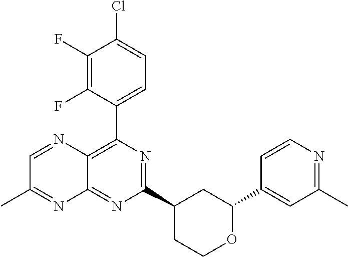 |
| I-126 | 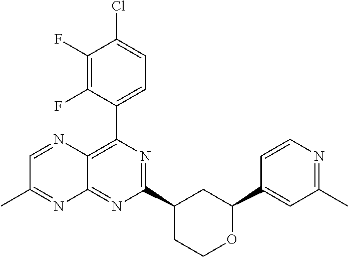 |
| I-127 | 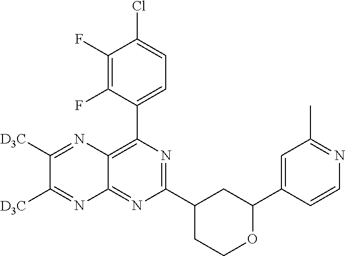 [SMILES: [2H]C([2H])([2H])c1nc2nc(C3CCOC(c4ccnc(C)c4)C3)nc(-c3ccc(Cl)c(F)c3F)c2nc1C([2H])([2H])[2H]] |
| I-128 | 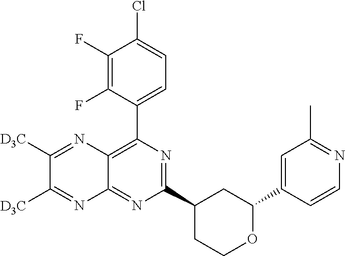 [SMILES: [2H]C([2H])([2H])c1nc2nc([C@@H]3CCO[C@@H](c4ccnc(C)c4)C3)nc(-c3ccc(Cl)c(F)c3F)c2nc1C([2H])([2H])[2H]] |
| I-129 | 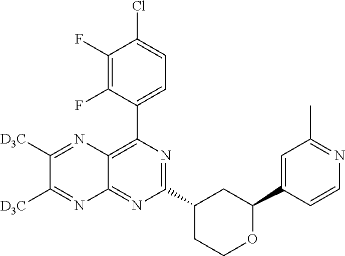 [SMILES: [2H]C([2H])([2H])c1nc2nc([C@H]3CCO[C@H](c4ccnc(C)c4)C3)nc(-c3ccc(Cl)c(F)c3F)c2nc1C([2H])([2H])[2H]] |
| I-130 | 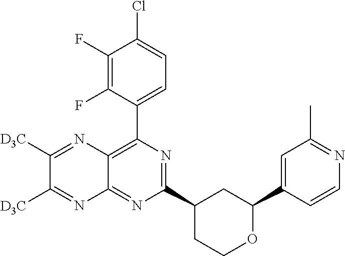 [SMILES: [2H]C([2H])([2H])c1nc2nc([C@@H]3CCO[C@H](c4ccnc(C)c4)C3)nc(-c3ccc(Cl)c(F)c3F)c2nc1C([2H])([2H])[2H]] |
| I-131 | 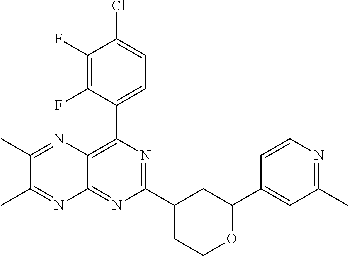 |
| I-132 | 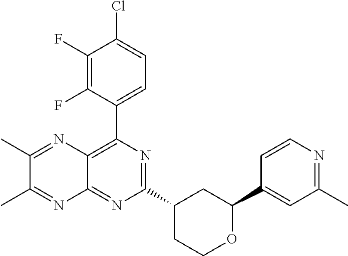 |
| I-133 | 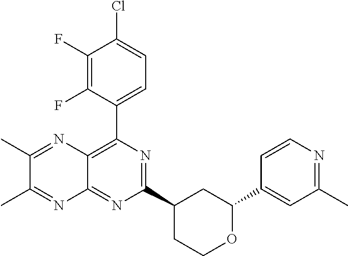 |
| I-134 | 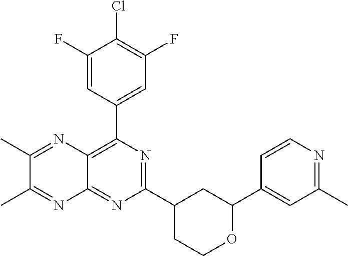 |
| I-135 | 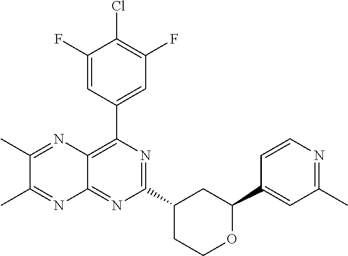 |
| I-136 | 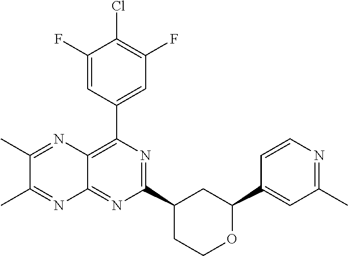 |
| I-137 | 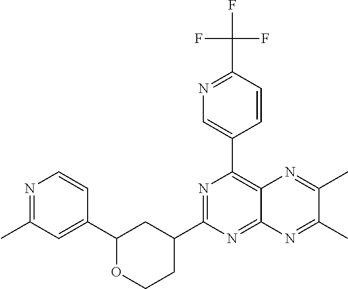 |
| I-138 | 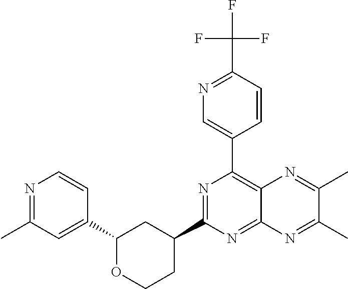 |
| I-139 | 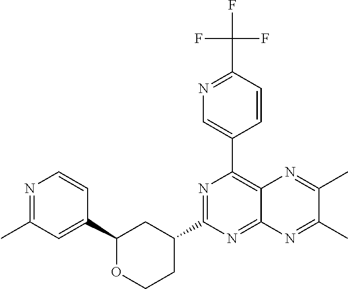 |
| I-140 | 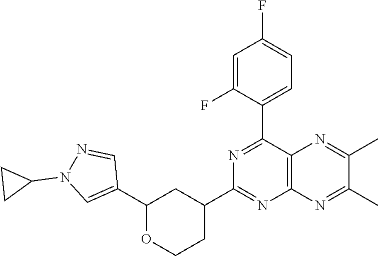 [SMILES: Cc1nc2nc(C3CCOC(c4cnn(C5CC5)c4)C3)nc(-c3ccc(F)cc3F)c2nc1C] |
| I-141 | 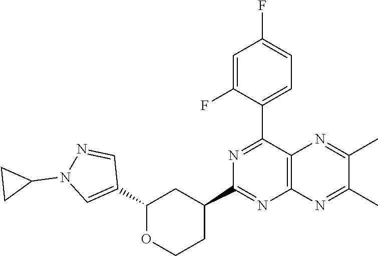 [SMILES: Cc1nc2nc([C@H]3CCO[C@H](c4cnn(C5CC5)c4)C3)nc(-c3ccc(F)cc3F)c2nc1C] |
| I-142 | 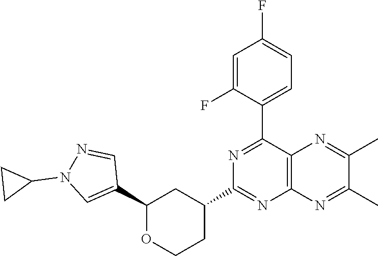 [SMILES: Cc1nc2nc([C@@H]3CCO[C@@H](c4cnn(C5CC5)c4)C3)nc(-c3ccc(F)cc3F)c2nc1C] |
| I-143 | 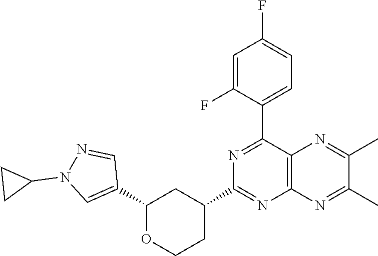 [SMILES: Cc1nc2nc([C@@H]3CCO[C@H](c4cnn(C5CC5)c4)C3)nc(-c3ccc(F)cc3F)c2nc1C] |
| I-144 | 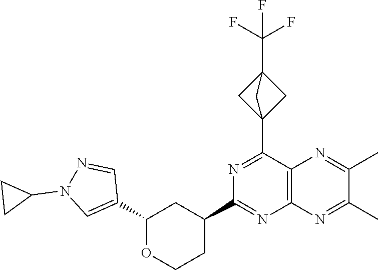 [SMILES: Cc1nc2nc([C@H]3CCO[C@H](c4cnn(C5CC5)c4)C3)nc(C34CC(C(F)(F)F)(C3)C4)c2nc1C] |
| I-145 | 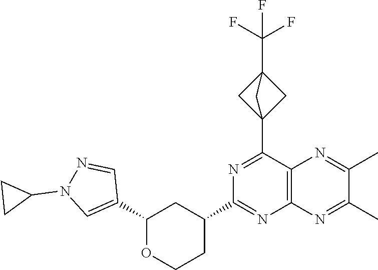 [SMILES: Cc1nc2nc([C@@H]3CCO[C@H](c4cnn(C5CC5)c4)C3)nc(C34CC(C(F)(F)F)(C3)C4)c2nc1C] |
| I-146 | 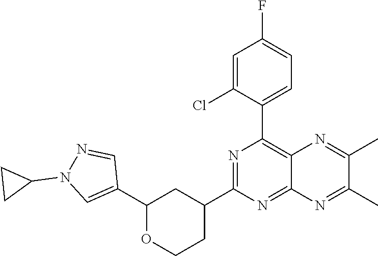 [SMILES: Cc1nc2nc(C3CCOC(c4cnn(C5CC5)c4)C3)nc(-c3ccc(F)cc3Cl)c2nc1C] |
| I-147 | 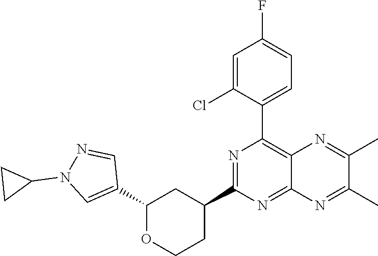 [SMILES: Cc1nc2nc([C@H]3CCO[C@H](c4cnn(C5CC5)c4)C3)nc(-c3ccc(F)cc3Cl)c2nc1C] |
| I-148 | 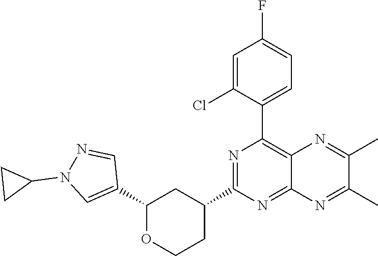 [SMILES: Cc1nc2nc([C@@H]3CCO[C@H](c4cnn(C5CC5)c4)C3)nc(-c3ccc(F)cc3Cl)c2nc1C] |
| I-149 | 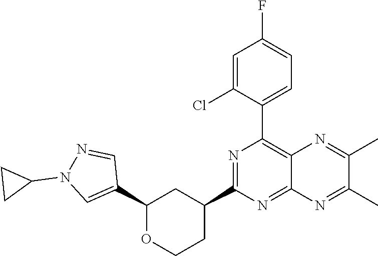 [SMILES: Cc1nc2nc([C@H]3CCO[C@@H](c4cnn(C5CC5)c4)C3)nc(-c3ccc(F)cc3Cl)c2nc1C] |
| I-150 | 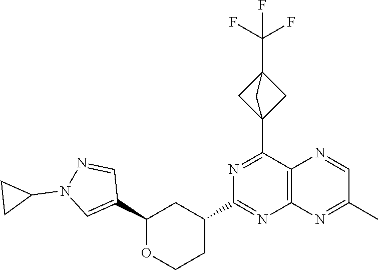 [SMILES: Cc1cnc2c(C34CC(C(F)(F)F)(C3)C4)nc([C@@H]3CCO[C@@H](c4cnn(C5CC5)c4)C3)nc2n1] |
| I-151 | 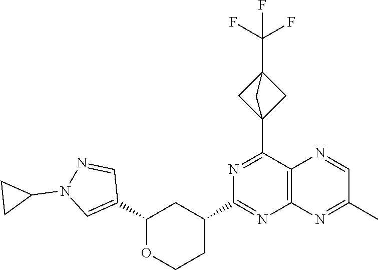 [SMILES: Cc1cnc2c(C34CC(C(F)(F)F)(C3)C4)nc([C@@H]3CCO[C@H](c4cnn(C5CC5)c4)C3)nc2n1] |
| I-152 | 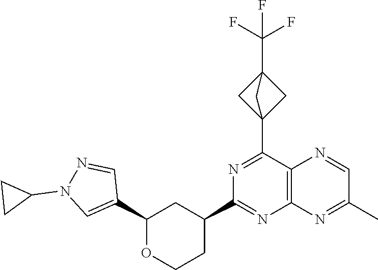 [SMILES: Cc1cnc2c(C34CC(C(F)(F)F)(C3)C4)nc([C@H]3CCO[C@@H](c4cnn(C5CC5)c4)C3)nc2n1] |
| I-153 | 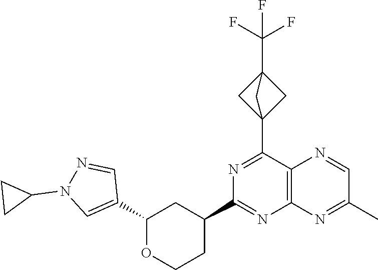 [SMILES: Cc1cnc2c(C34CC(C(F)(F)F)(C3)C4)nc([C@H]3CCO[C@H](c4cnn(C5CC5)c4)C3)nc2n1] |
| I-154 | 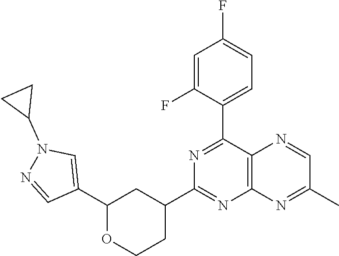 [SMILES: Cc1cnc2c(-c3ccc(F)cc3F)nc(C3CCOC(c4cnn(C5CC5)c4)C3)nc2n1] |
| I-155 | 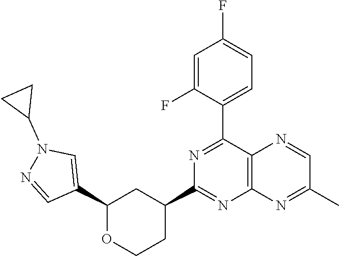 [SMILES: Cc1cnc2c(-c3ccc(F)cc3F)nc([C@H]3CCO[C@@H](c4cnn(C5CC5)c4)C3)nc2n1] |
| I-156 | 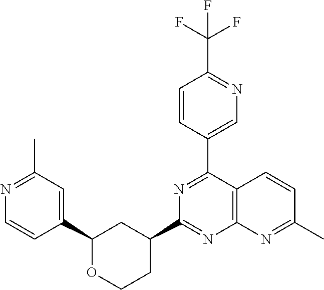 [SMILES: Cc1cc([C@H]2C[C@@H](c3nc(-c4ccc(C(F)(F)F)nc4)c4ccc(C)nc4n3)CCO2)ccn1] |
| I-157 | 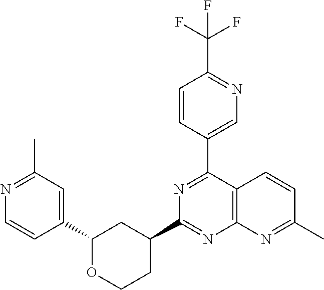 [SMILES: Cc1cc([C@@H]2C[C@@H](c3nc(-c4ccc(C(F)(F)F)nc4)c4ccc(C)nc4n3)CCO2)ccn1] |
| I-158 | 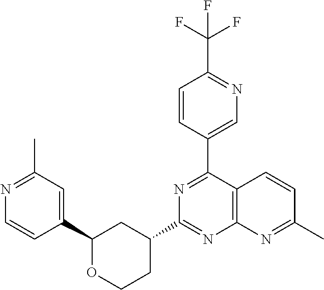 [SMILES: Cc1cc([C@H]2C[C@H](c3nc(-c4ccc(C(F)(F)F)nc4)c4ccc(C)nc4n3)CCO2)ccn1] |
| I-159 | 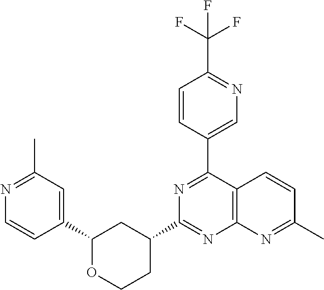 [SMILES: Cc1cc([C@@H]2C[C@H](c3nc(-c4ccc(C(F)(F)F)nc4)c4ccc(C)nc4n3)CCO2)ccn1] |
| I-160 | 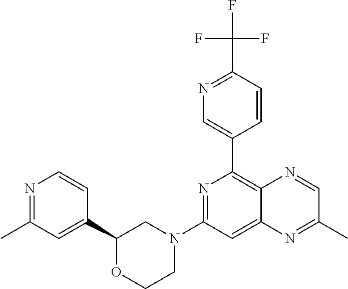 |
| I-161 | 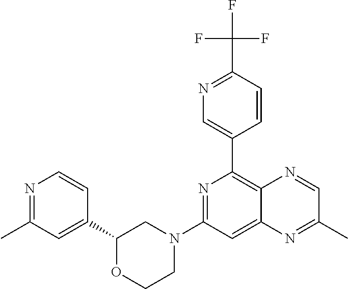 |
| I-162 | 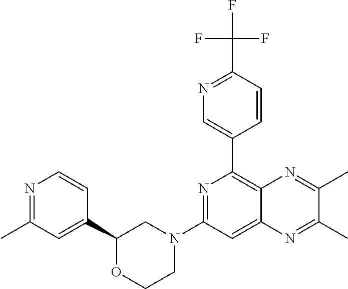 |
| I-163 | 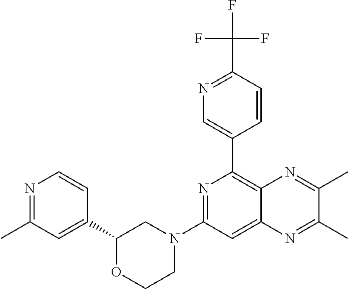 |
| I-164 | 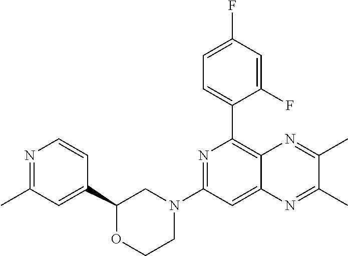 |
| I-165 | 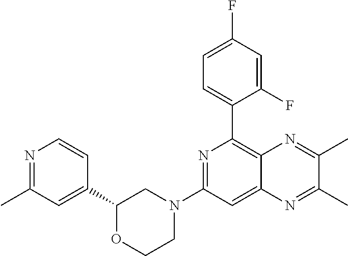 |
| I-166 | 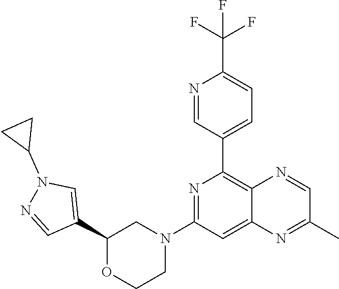 [SMILES: Cc1cnc2c(-c3ccc(C(F)(F)F)nc3)nc(N3CCO[C@@H](c4cnn(C5CC5)c4)C3)cc2n1] |
| I-167 | 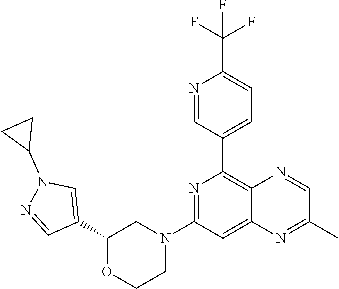 [SMILES: Cc1cnc2c(-c3ccc(C(F)(F)F)nc3)nc(N3CCO[C@H](c4cnn(C5CC5)c4)C3)cc2n1] |
| I-168 | 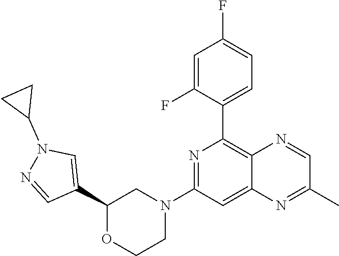 [SMILES: Cc1cnc2c(-c3ccc(F)cc3F)nc(N3CCO[C@@H](c4cnn(C5CC5)c4)C3)cc2n1] |
| I-169 | 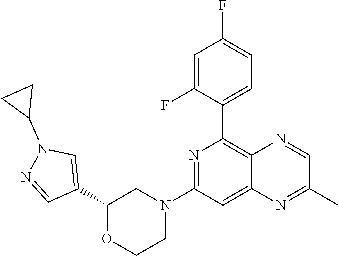 [SMILES: Cc1cnc2c(-c3ccc(F)cc3F)nc(N3CCO[C@H](c4cnn(C5CC5)c4)C3)cc2n1] |
| I-170 | 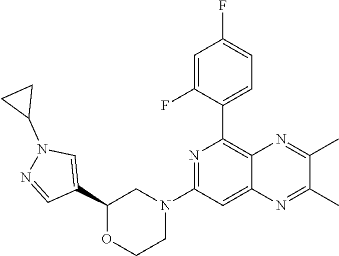 [SMILES: Cc1nc2cc(N3CCO[C@@H](c4cnn(C5CC5)c4)C3)nc(-c3ccc(F)cc3F)c2nc1C] |
| I-171 | 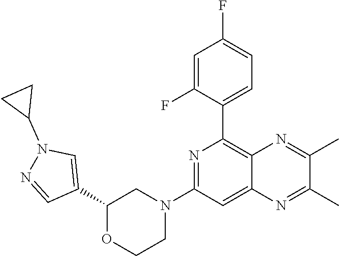 [SMILES: Cc1nc2cc(N3CCO[C@H](c4cnn(C5CC5)c4)C3)nc(-c3ccc(F)cc3F)c2nc1C] |
| I-172 | 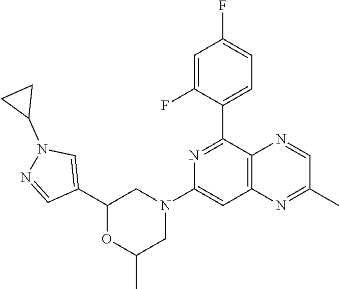 [SMILES: Cc1cnc2c(-c3ccc(F)cc3F)nc(N3CC(C)OC(c4cnn(C5CC5)c4)C3)cc2n1] |
| I-173 | 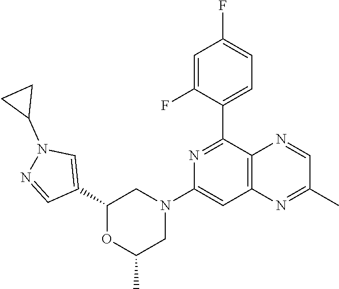 [SMILES: Cc1cnc2c(-c3ccc(F)cc3F)nc(N3C[C@@H](c4cnn(C5CC5)c4)O[C@@H](C)C3)cc2n1] |
| I-174 | 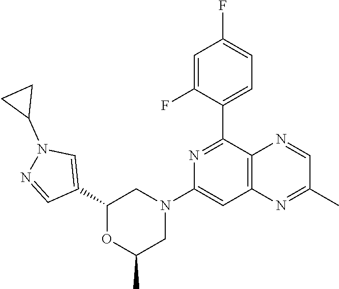 [SMILES: Cc1cnc2c(-c3ccc(F)cc3F)nc(N3C[C@@H](C)O[C@H](c4cnn(C5CC5)c4)C3)cc2n1] |
| I-175 | 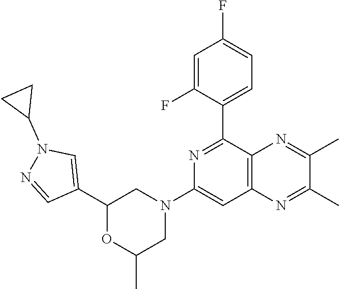 [SMILES: Cc1nc2cc(N3CC(C)OC(c4cnn(C5CC5)c4)C3)nc(-c3ccc(F)cc3F)c2nc1C] |
| I-176 | 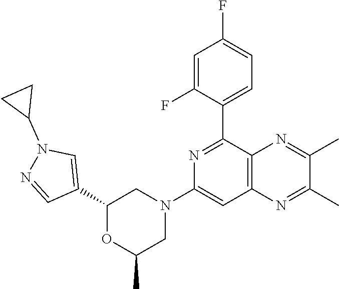 [SMILES: Cc1nc2cc(N3C[C@@H](C)O[C@H](c4cnn(C5CC5)c4)C3)nc(-c3ccc(F)cc3F)c2nc1C] |
| I-177 | 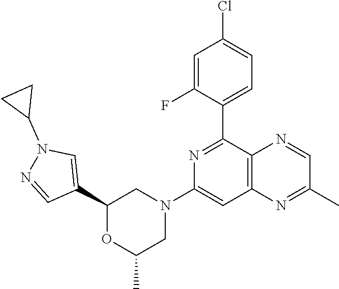 [SMILES: Cc1cnc2c(-c3ccc(Cl)cc3F)nc(N3C[C@H](C)O[C@@H](c4cnn(C5CC5)c4)C3)cc2n1] |
| I-178 | 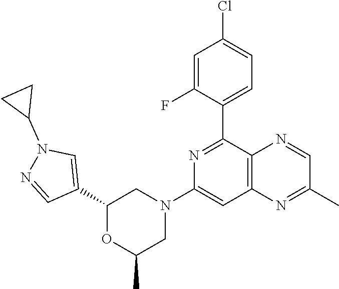 [SMILES: Cc1cnc2c(-c3ccc(Cl)cc3F)nc(N3C[C@@H](C)O[C@H](c4cnn(C5CC5)c4)C3)cc2n1] |
| I-179 | 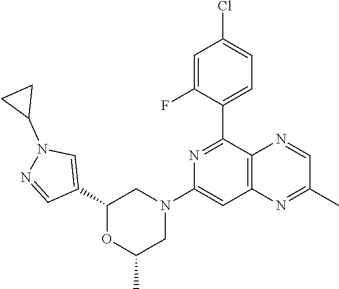 [SMILES: Cc1cnc2c(-c3ccc(Cl)cc3F)nc(N3C[C@@H](c4cnn(C5CC5)c4)O[C@@H](C)C3)cc2n1] |
| I-180 | 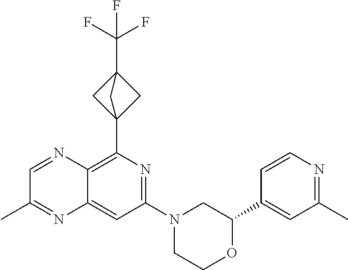 |
| I-181 | 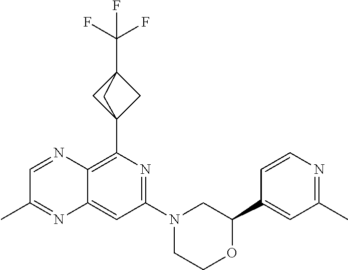 |
| I-182 | 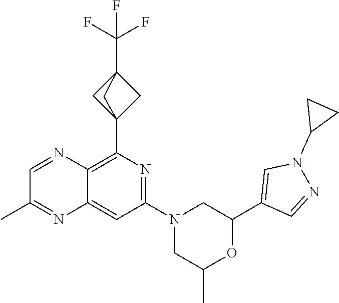 [SMILES: Cc1cnc2c(C34CC(C(F)(F)F)(C3)C4)nc(N3CC(C)OC(c4cnn(C5CC5)c4)C3)cc2n1] |
| I-183 | 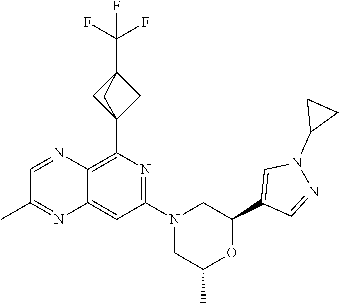 [SMILES: Cc1cnc2c(C34CC(C(F)(F)F)(C3)C4)nc(N3C[C@@H](C)O[C@H](c4cnn(C5CC5)c4)C3)cc2n1] |
| I-184 | 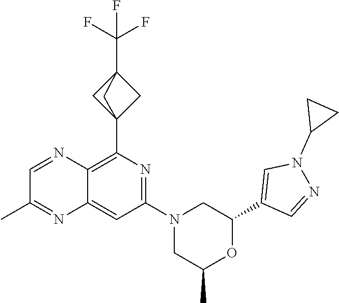 [SMILES: Cc1cnc2c(C34CC(C(F)(F)F)(C3)C4)nc(N3C[C@H](C)O[C@@H](c4cnn(C5CC5)c4)C3)cc2n1] |
| I-185 | 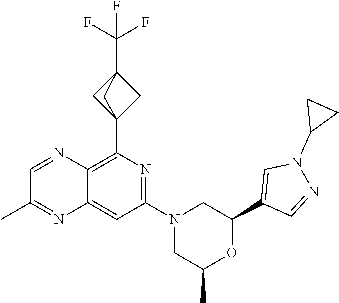 [SMILES: Cc1cnc2c(C34CC(C(F)(F)F)(C3)C4)nc(N3C[C@@H](c4cnn(C5CC5)c4)O[C@@H](C)C3)cc2n1] |
| I-186 | 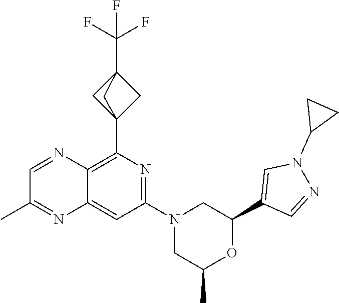 [SMILES: Cc1cnc2c(C34CC(C(F)(F)F)(C3)C4)nc(N3C[C@@H](c4cnn(C5CC5)c4)O[C@@H](C)C3)cc2n1] |
| I-187 | 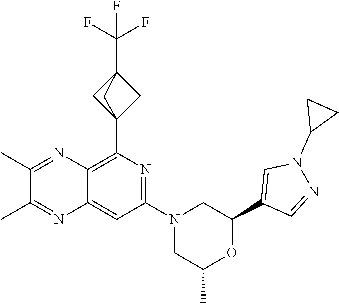 [SMILES: Cc1nc2cc(N3C[C@@H](C)O[C@H](c4cnn(C5CC5)c4)C3)nc(C34CC(C(F)(F)F)(C3)C4)c2nc1C] |
| I-188 | 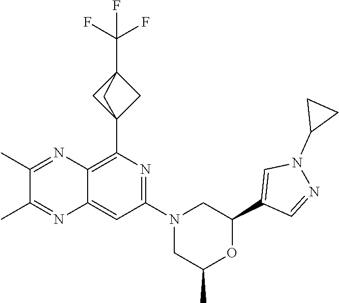 [SMILES: Cc1nc2cc(N3C[C@@H](c4cnn(C5CC5)c4)O[C@@H](C)C3)nc(C34CC(C(F)(F)F)(C3)C4)c2nc1C] |
| I-189 | 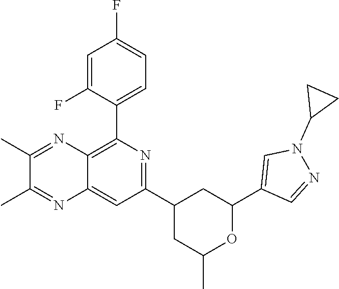 [SMILES: Cc1nc2cc(C3CC(C)OC(c4cnn(C5CC5)c4)C3)nc(-c3ccc(F)cc3F)c2nc1C] |
| I-190 | 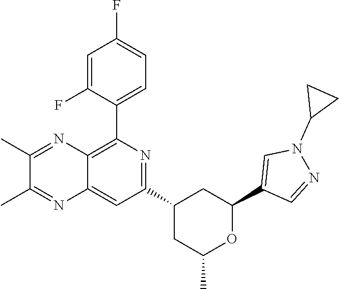 [SMILES: Cc1nc2cc([C@H]3C[C@@H](C)O[C@H](c4cnn(C5CC5)c4)C3)nc(-c3ccc(F)cc3F)c2nc1C] |
| I-191 | 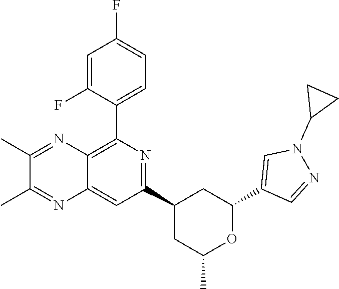 [SMILES: Cc1nc2cc([C@@H]3C[C@@H](C)O[C@@H](c4cnn(C5CC5)c4)C3)nc(-c3ccc(F)cc3F)c2nc1C] |
| I-192 | 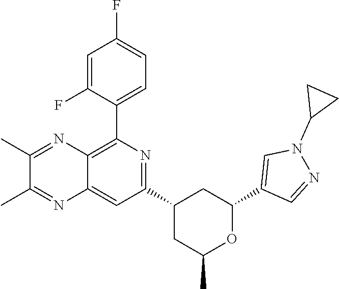 [SMILES: Cc1nc2cc([C@H]3C[C@H](C)O[C@@H](c4cnn(C5CC5)c4)C3)nc(-c3ccc(F)cc3F)c2nc1C] |
| I-193 | 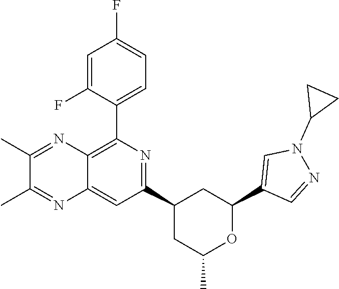 [SMILES: Cc1nc2cc([C@@H]3C[C@@H](C)O[C@H](c4cnn(C5CC5)c4)C3)nc(-c3ccc(F)cc3F)c2nc1C] |
| I-194 | 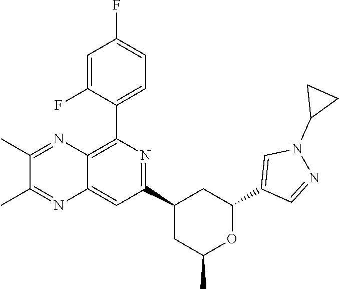 [SMILES: Cc1nc2cc([C@@H]3C[C@H](C)O[C@@H](c4cnn(C5CC5)c4)C3)nc(-c3ccc(F)cc3F)c2nc1C] |
| I-195 | 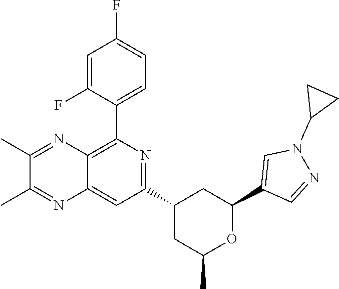 [SMILES: Cc1nc2cc([C@@H]3C[C@@H](c4cnn(C5CC5)c4)O[C@@H](C)C3)nc(-c3ccc(F)cc3F)c2nc1C] |
| I-196 | 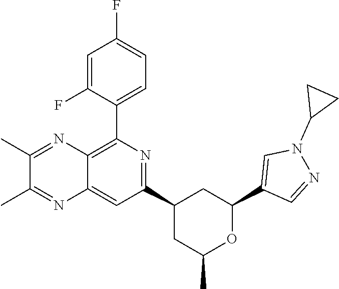 [SMILES: Cc1nc2cc([C@H]3C[C@@H](c4cnn(C5CC5)c4)O[C@@H](C)C3)nc(-c3ccc(F)cc3F)c2nc1C] |
| I-197 | 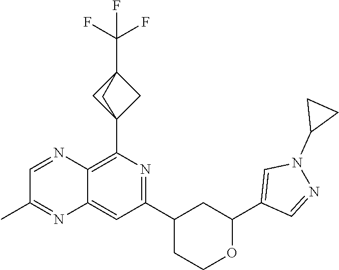 [SMILES: Cc1cnc2c(C34CC(C(F)(F)F)(C3)C4)nc(C3CCOC(c4cnn(C5CC5)c4)C3)cc2n1] |
| I-198 | 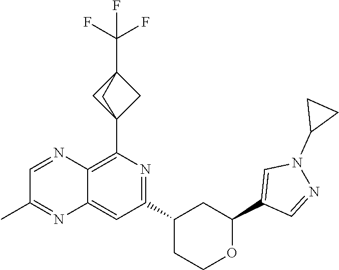 [SMILES: Cc1cnc2c(C34CC(C(F)(F)F)(C3)C4)nc([C@H]3CCO[C@H](c4cnn(C5CC5)c4)C3)cc2n1] |
| I-199 | 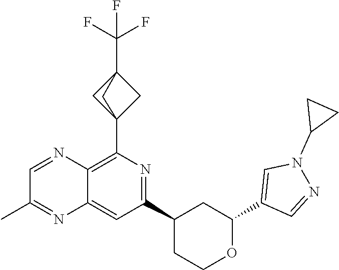 [SMILES: Cc1cnc2c(C34CC(C(F)(F)F)(C3)C4)nc([C@@H]3CCO[C@@H](c4cnn(C5CC5)c4)C3)cc2n1] |
| I-200 | 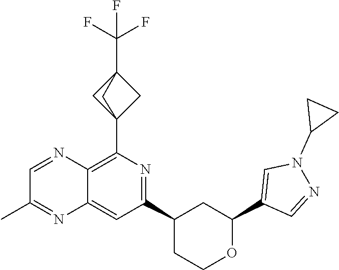 [SMILES: Cc1cnc2c(C34CC(C(F)(F)F)(C3)C4)nc([C@@H]3CCO[C@H](c4cnn(C5CC5)c4)C3)cc2n1] |
| I-201 | 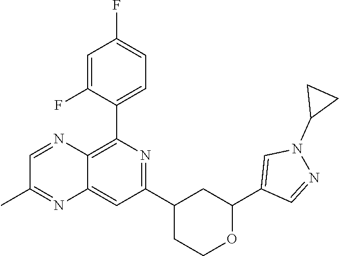 [SMILES: Cc1cnc2c(-c3ccc(F)cc3F)nc(C3CCOC(c4cnn(C5CC5)c4)C3)cc2n1] |
| I-202 | 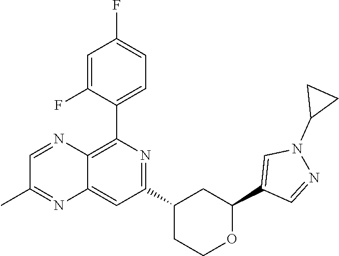 [SMILES: Cc1cnc2c(-c3ccc(F)cc3F)nc([C@H]3CCO[C@H](c4cnn(C5CC5)c4)C3)cc2n1] |
| I-203 | 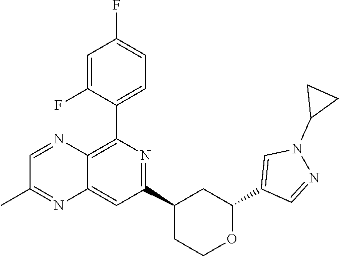 [SMILES: Cc1cnc2c(-c3ccc(F)cc3F)nc([C@@H]3CCO[C@@H](c4cnn(C5CC5)c4)C3)cc2n1] |
| I-204 | 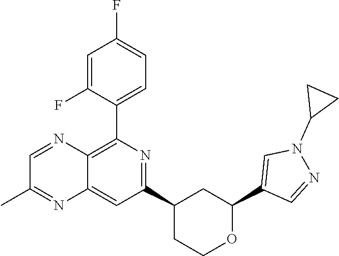 [SMILES: Cc1cnc2c(-c3ccc(F)cc3F)nc([C@@H]3CCO[C@H](c4cnn(C5CC5)c4)C3)cc2n1] |
| I-205 | 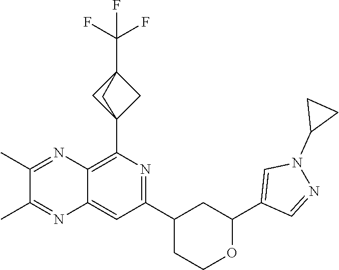 [SMILES: Cc1nc2cc(C3CCOC(c4cnn(C5CC5)c4)C3)nc(C34CC(C(F)(F)F)(C3)C4)c2nc1C] |
| I-206 | 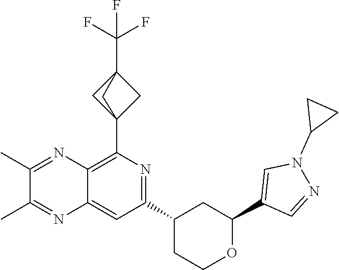 [SMILES: Cc1nc2cc([C@H]3CCO[C@H](c4cnn(C5CC5)c4)C3)nc(C34CC(C(F)(F)F)(C3)C4)c2nc1C] |
| I-207 | 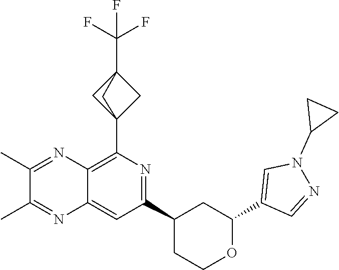 [SMILES: Cc1nc2cc([C@@H]3CCO[C@@H](c4cnn(C5CC5)c4)C3)nc(C34CC(C(F)(F)F)(C3)C4)c2nc1C] |
| I-208 | 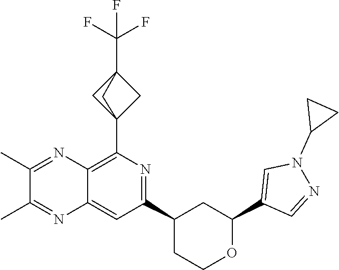 [SMILES: Cc1nc2cc([C@@H]3CCO[C@H](c4cnn(C5CC5)c4)C3)nc(C34CC(C(F)(F)F)(C3)C4)c2nc1C] |
| I-209 | 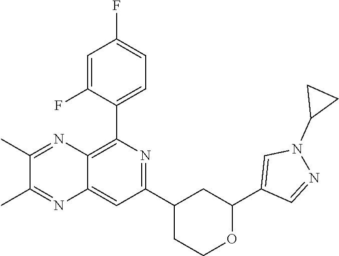 [SMILES: Cc1nc2cc(C3CCOC(c4cnn(C5CC5)c4)C3)nc(-c3ccc(F)cc3F)c2nc1C] |
| I-210 | 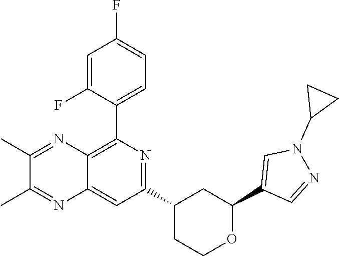 [SMILES: Cc1nc2cc([C@H]3CCO[C@H](c4cnn(C5CC5)c4)C3)nc(-c3ccc(F)cc3F)c2nc1C] |
| I-211 | 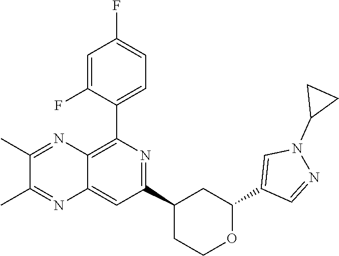 [SMILES: Cc1nc2cc([C@@H]3CCO[C@@H](c4cnn(C5CC5)c4)C3)nc(-c3ccc(F)cc3F)c2nc1C] |
| I-212 | 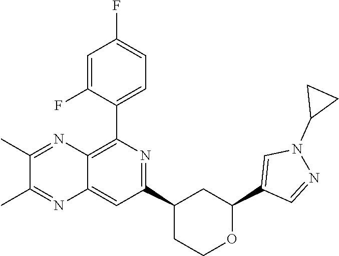 [SMILES: Cc1nc2cc([C@@H]3CCO[C@H](c4cnn(C5CC5)c4)C3)nc(-c3ccc(F)cc3F)c2nc1C] |
| I-213 | 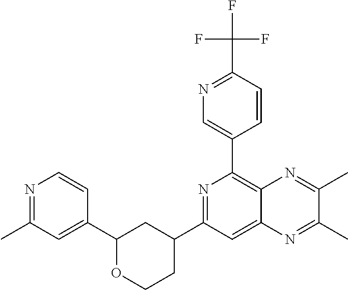 |
| I-214 | 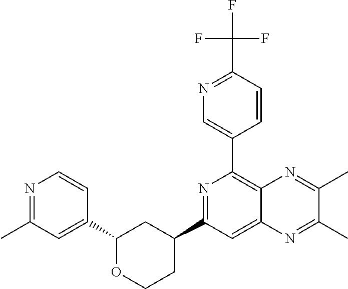 |
| I-215 | 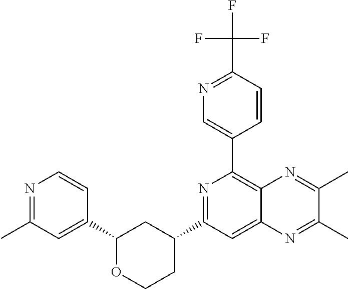 |
| I-216 | 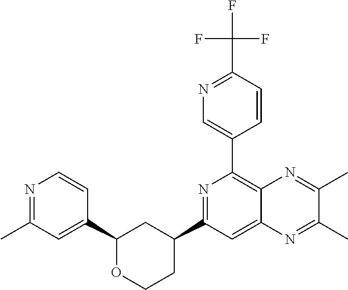 |
| I-217 | 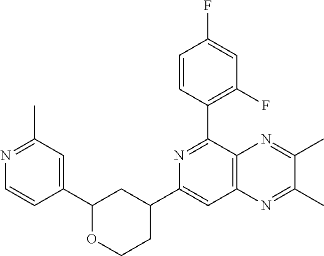 [SMILES: Cc1cc(C2CC(c3cc4nc(C)c(C)nc4c(-c4ccc(F)cc4F)n3)CCO2)ccn1] |
| I-218 | 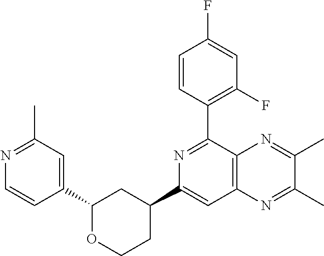 [SMILES: Cc1cc([C@@H]2C[C@@H](c3cc4nc(C)c(C)nc4c(-c4ccc(F)cc4F)n3)CCO2)ccn1] |
| I-219 | 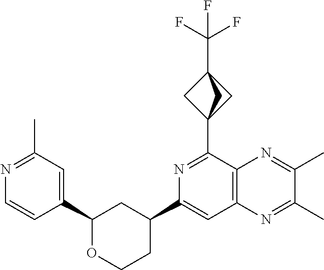 [SMILES: Cc1cc([C@H]2C[C@@H](c3cc4nc(C)c(C)nc4c([C@]45C[C@](C(F)(F)F)(C4)C5)n3)CCO2)ccn1] |
| I-220 | 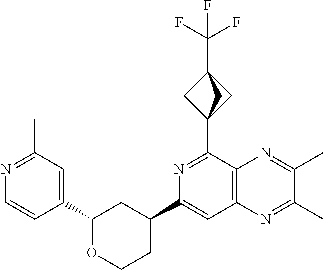 [SMILES: Cc1cc([C@@H]2C[C@@H](c3cc4nc(C)c(C)nc4c([C@]45C[C@](C(F)(F)F)(C4)C5)n3)CCO2)ccn1] |
| I-221 | 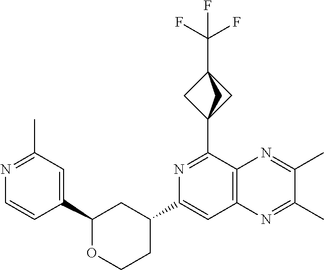 [SMILES: Cc1cc([C@H]2C[C@H](c3cc4nc(C)c(C)nc4c([C@]45C[C@](C(F)(F)F)(C4)C5)n3)CCO2)ccn1] |
| I-222 | 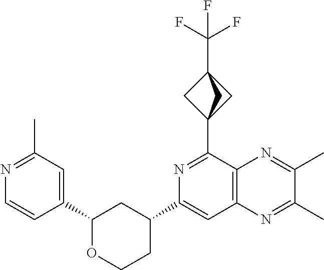 [SMILES: Cc1cc([C@@H]2C[C@H](c3cc4nc(C)c(C)nc4c([C@]45C[C@](C(F)(F)F)(C4)C5)n3)CCO2)ccn1] |
| I-223 | 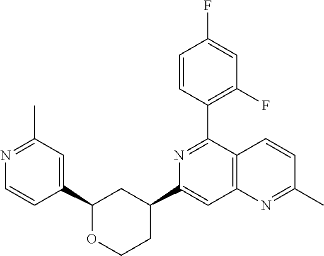 [SMILES: Cc1cc([C@H]2C[C@@H](c3cc4nc(C)ccc4c(-c4ccc(F)cc4F)n3)CCO2)ccn1] |
| I-224 | 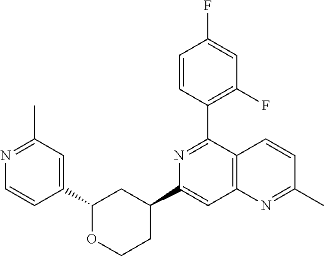 [SMILES: Cc1cc([C@@H]2C[C@@H](c3cc4nc(C)ccc4c(-c4ccc(F)cc4F)n3)CCO2)ccn1] |
| I-225 | 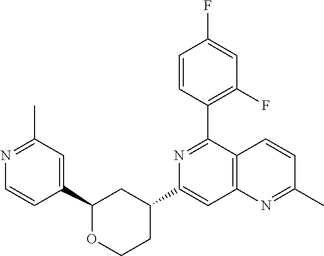 [SMILES: Cc1cc([C@H]2C[C@H](c3cc4nc(C)ccc4c(-c4ccc(F)cc4F)n3)CCO2)ccn1] |
| I-226 | 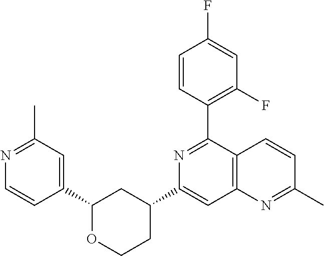 [SMILES: Cc1cc([C@@H]2C[C@H](c3cc4nc(C)ccc4c(-c4ccc(F)cc4F)n3)CCO2)ccn1] |
| I-227 | 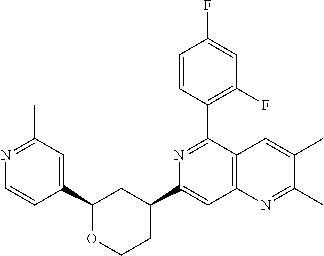 [SMILES: Cc1cc([C@H]2C[C@@H](c3cc4nc(C)c(C)cc4c(-c4ccc(F)cc4F)n3)CCO2)ccn1] |
| I-228 | 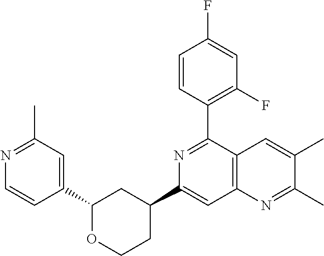 [SMILES: Cc1cc([C@@H]2C[C@@H](c3cc4nc(C)c(C)cc4c(-c4ccc(F)cc4F)n3)CCO2)ccn1] |
| I-229 | 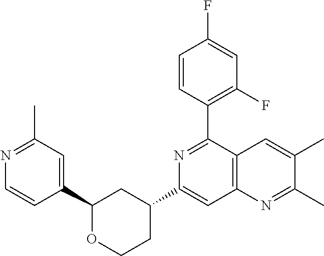 [SMILES: Cc1cc([C@H]2C[C@H](c3cc4nc(C)c(C)cc4c(-c4ccc(F)cc4F)n3)CCO2)ccn1] |
| I-230 | 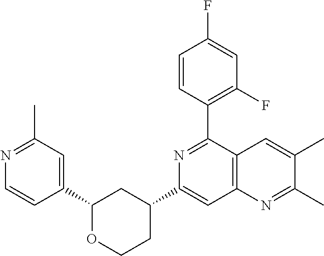 [SMILES: Cc1cc([C@@H]2C[C@H](c3cc4nc(C)c(C)cc4c(-c4ccc(F)cc4F)n3)CCO2)ccn1] |
| I-231 | 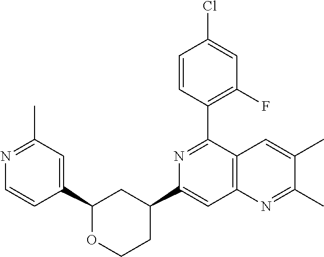 [SMILES: Cc1cc([C@H]2C[C@@H](c3cc4nc(C)c(C)cc4c(-c4ccc(Cl)cc4F)n3)CCO2)ccn1] |
| I-232 | 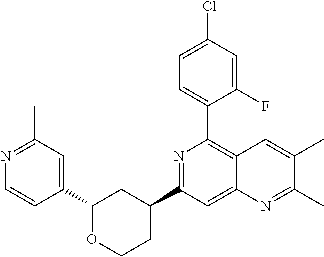 [SMILES: Cc1cc([C@@H]2C[C@@H](c3cc4nc(C)c(C)cc4c(-c4ccc(Cl)cc4F)n3)CCO2)ccn1] |
| I-233 | 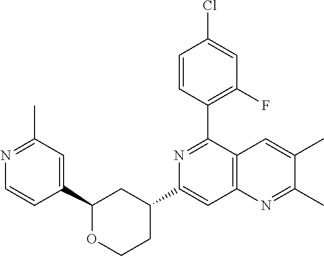 [SMILES: Cc1cc([C@H]2C[C@H](c3cc4nc(C)c(C)cc4c(-c4ccc(Cl)cc4F)n3)CCO2)ccn1] |
| I-234 | 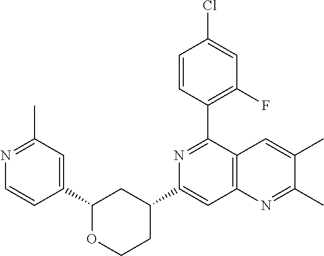 [SMILES: Cc1cc([C@@H]2C[C@H](c3cc4nc(C)c(C)cc4c(-c4ccc(Cl)cc4F)n3)CCO2)ccn1] |
| I-235 | 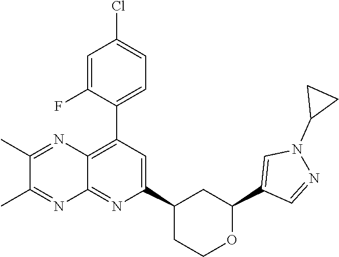 [SMILES: Cc1nc2nc([C@@H]3CCO[C@H](c4cnn(C5CC5)c4)C3)cc(-c3ccc(Cl)cc3F)c2nc1C] |
| I-236 | 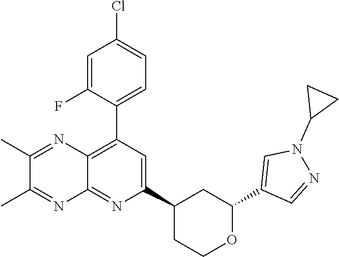 [SMILES: Cc1nc2nc([C@@H]3CCO[C@@H](c4cnn(C5CC5)c4)C3)cc(-c3ccc(Cl)cc3F)c2nc1C] |
| I-237 | 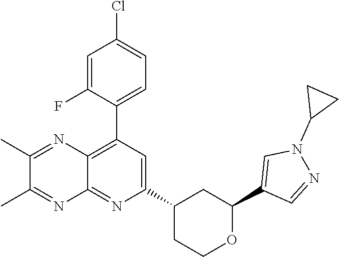 [SMILES: Cc1nc2nc([C@H]3CCO[C@H](c4cnn(C5CC5)c4)C3)cc(-c3ccc(Cl)cc3F)c2nc1C] |
| I-238 | 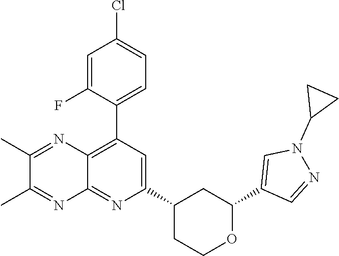 [SMILES: Cc1nc2nc([C@H]3CCO[C@@H](c4cnn(C5CC5)c4)C3)cc(-c3ccc(Cl)cc3F)c2nc1C] |
| I-239 | 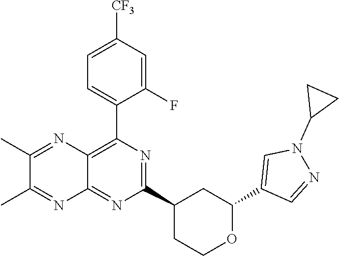 |
| I-240 | 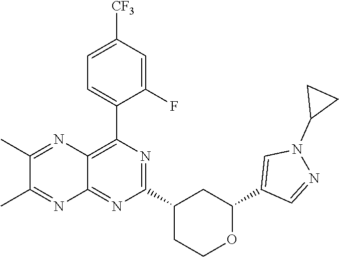 |
| I-241 | 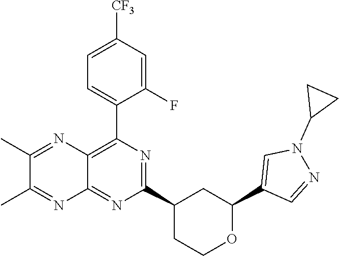 |
| I-242 | 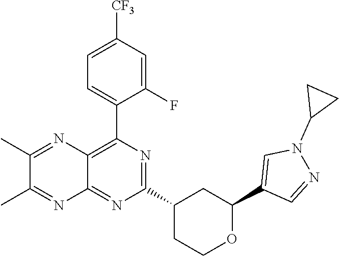 |
| I-243 | 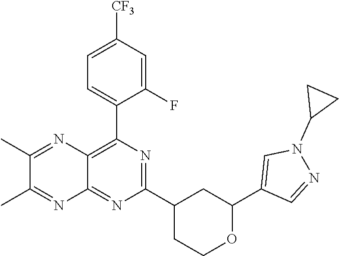 |
| I-244 | 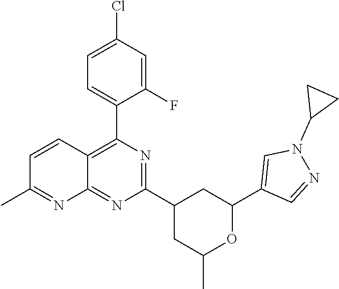 |
| I-245 | 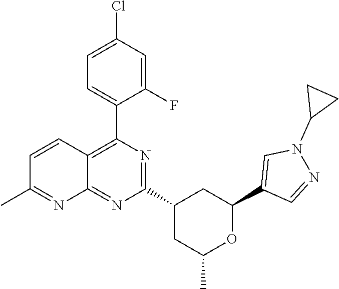 |
| I-246 | 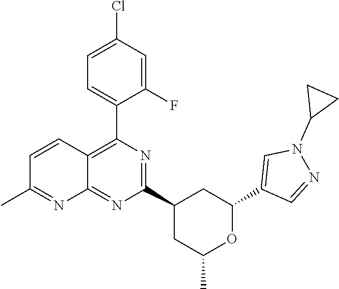 |
| I-247 | 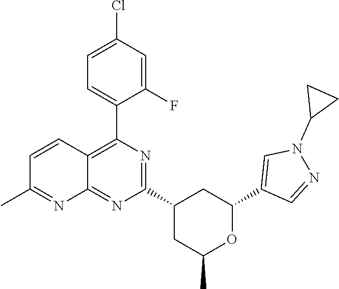 |
| I-248 | 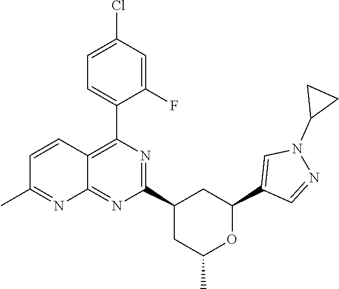 |
| I-249 | 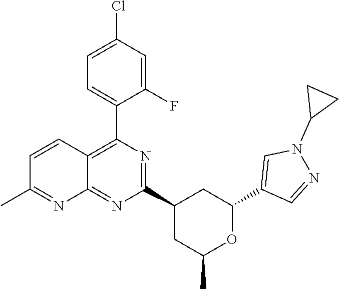 |
| I-250 | 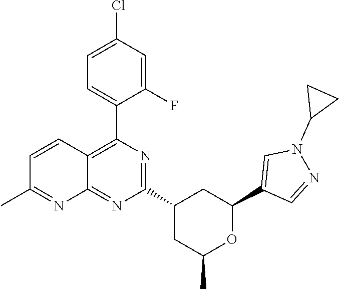 |
| I-251 | 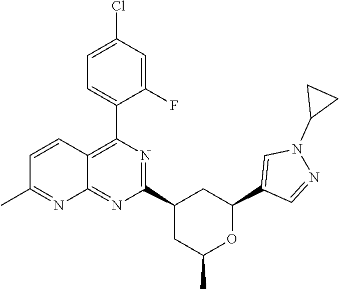 |
| I-252 | 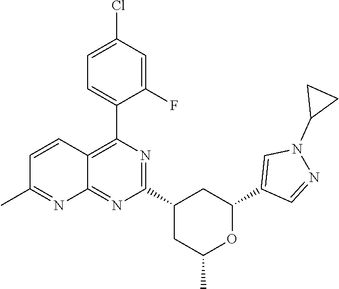 |
| I-253 | 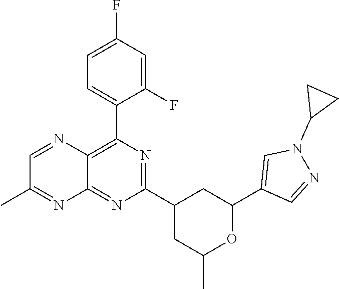 |
| I-254 | 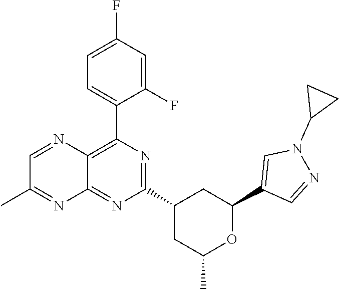 |
| I-255 | 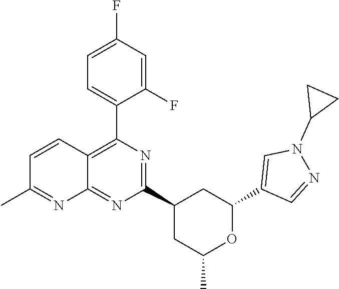 |
| I-256 | 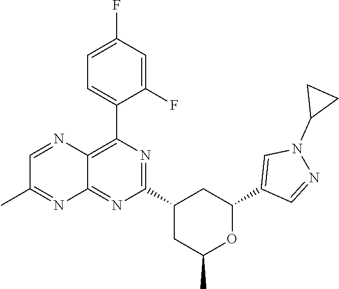 |
| I-257 | 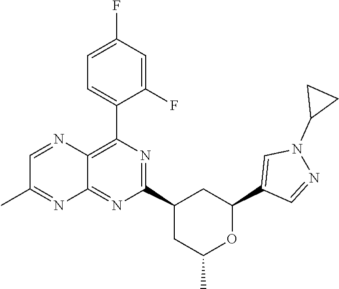 |
| I-258 | 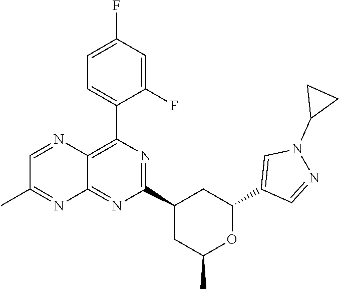 |
| I-259 | 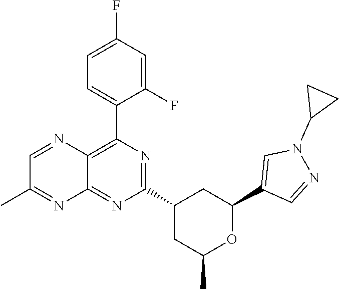 |
| I-260 | 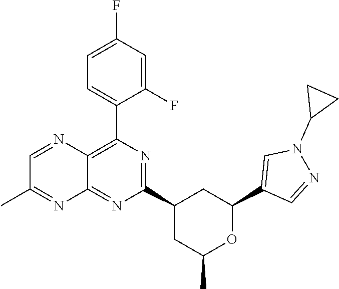 |
| I-261 | 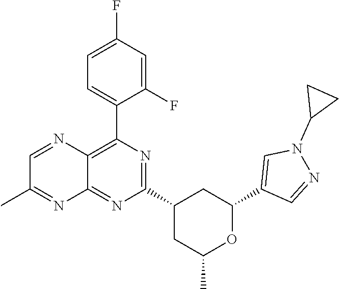 |
| I-262 | 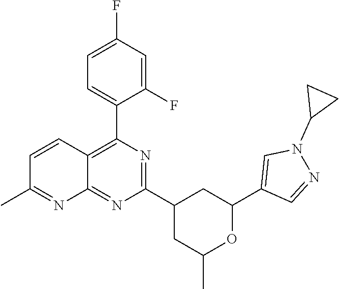 |
| I-263 | 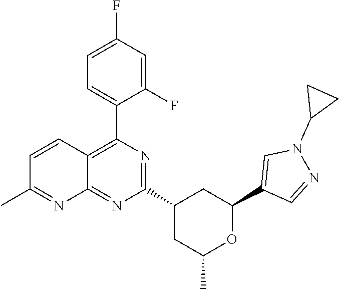 |
| I-264 | 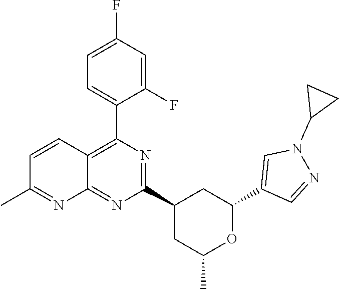 |
| I-265 | 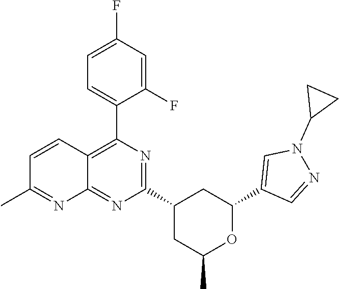 |
| I-266 | 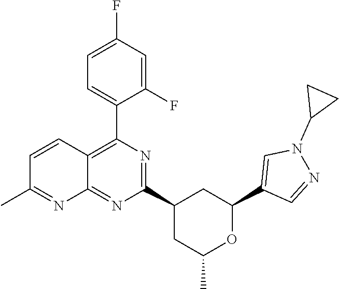 |
| I-267 | 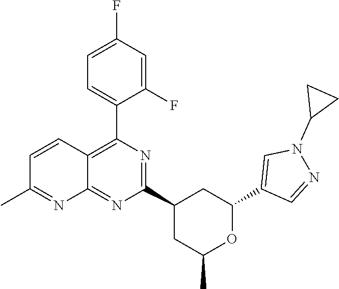 |
| I-268 | 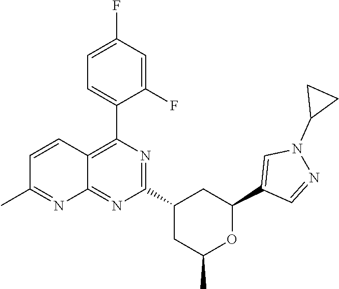 |
| I-269 | 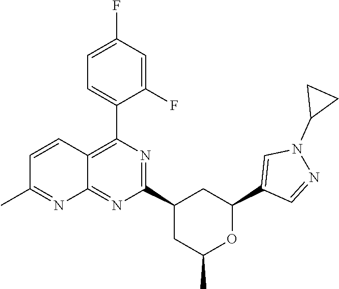 |
| I-270 | 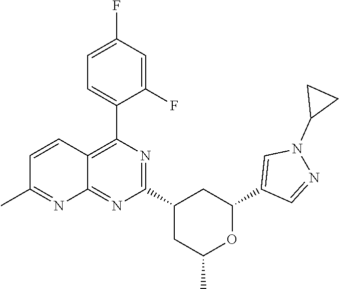 |
| I-271 | 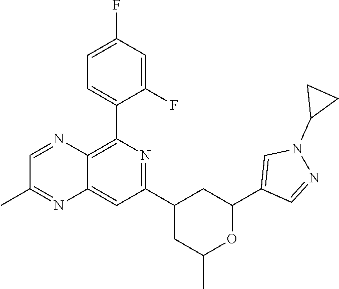 |
| I-272 | 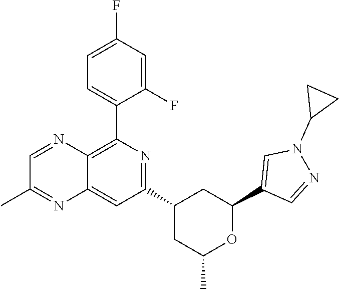 |
| I-273 | 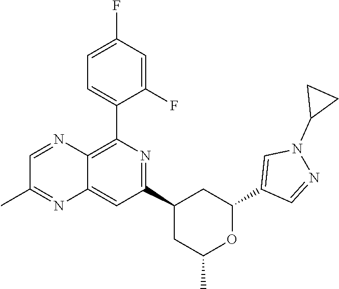 |
| I-274 | 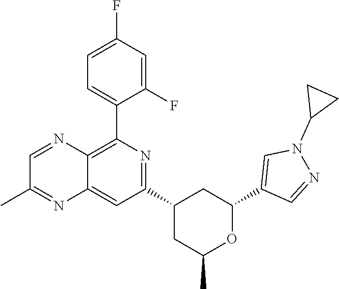 |
| I-275 | 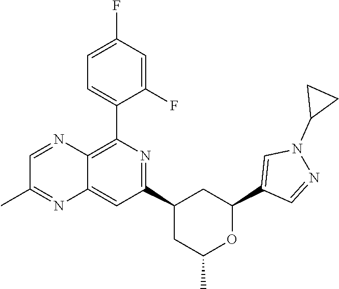 |
| I-276 | 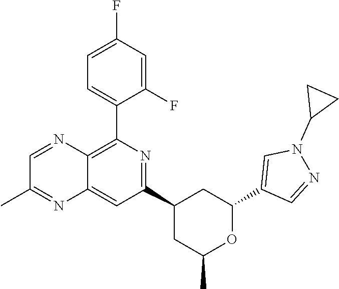 |
| I-277 | 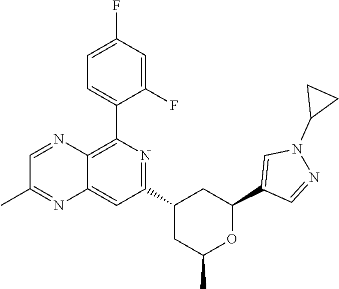 |
| I-278 | 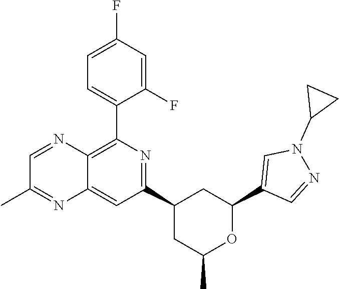 |
| I-279 | 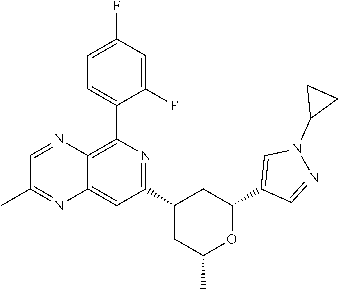 |
| I-280 | 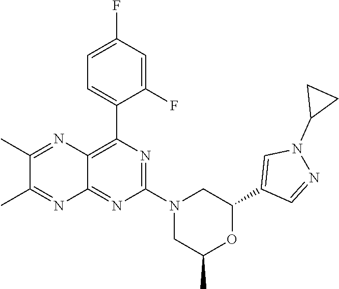 |
| I-281 | 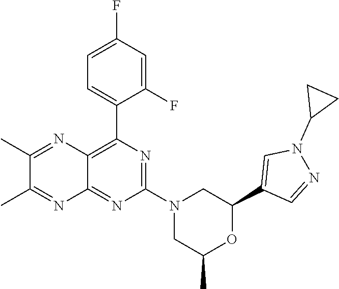 |
| I-282 | 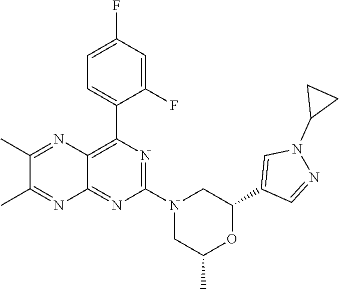 |
| I-283 | 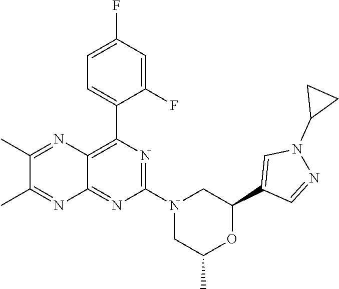 |
| I-284 | 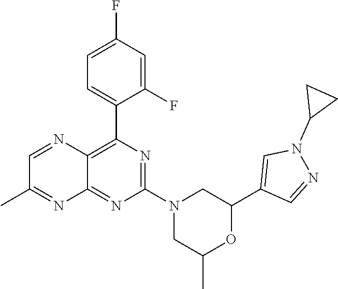 [SMILES: Cc1cnc2c(-c3ccc(F)cc3F)nc(N3CC(C)OC(c4cnn(C5CC5)c4)C3)nc2n1] |
| I-285 | 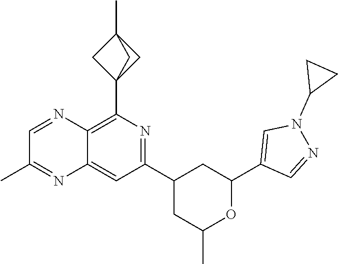 |
| I-286 | 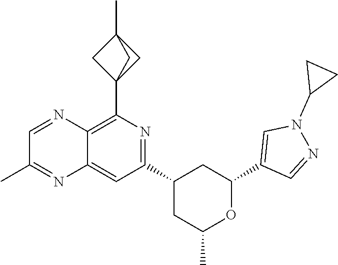 |
| I-287 | 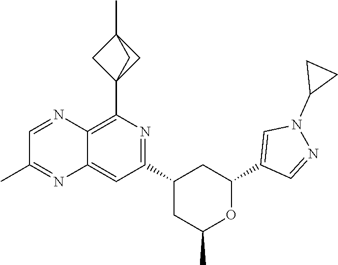 |
| I-288 | 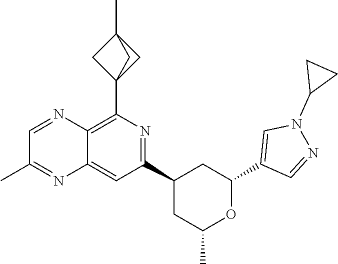 [SMILES: Cc1cnc2c(C34CC(C)(C3)C4)nc([C@@H]3C[C@@H](C)O[C@@H](c4cnn(C5CC5)c4)C3)cc2n1] |
| I-289 | 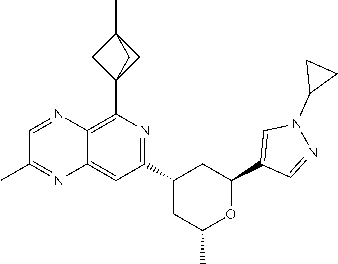 |
| I-290 | 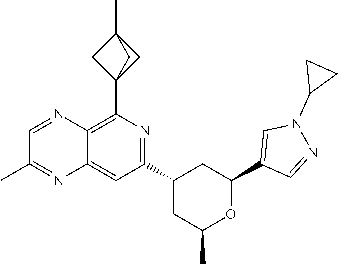 |
| I-291 | 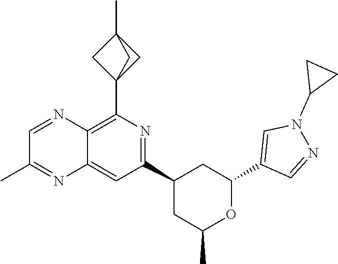 [SMILES: Cc1cnc2c(C34CC(C)(C3)C4)nc([C@@H]3C[C@H](C)O[C@@H](c4cnn(C5CC5)c4)C3)cc2n1] |
| I-292 | 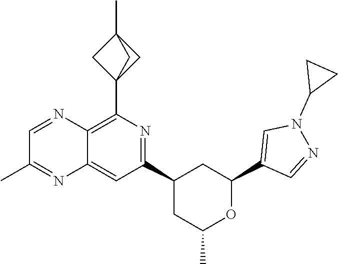 |
| I-293 | 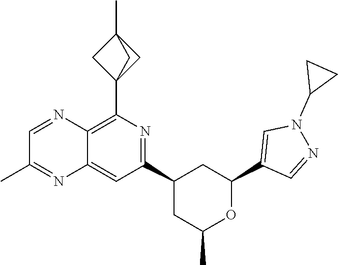 |
| I-294 | 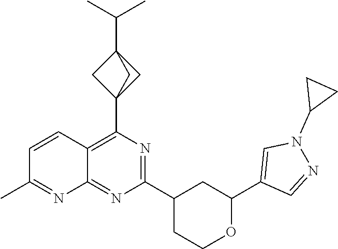 [SMILES: Cc1ccc2c(C34CC(C(C)C)(C3)C4)nc(C3CCOC(c4cnn(C5CC5)c4)C3)nc2n1] |
| I-295 | 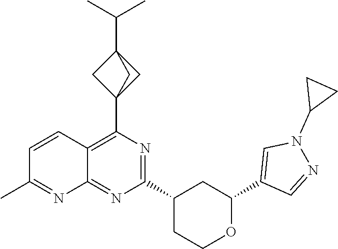 [SMILES: Cc1ccc2c(C34CC(C(C)C)(C3)C4)nc([C@H]3CCO[C@@H](c4cnn(C5CC5)c4)C3)nc2n1] |
| I-296 | 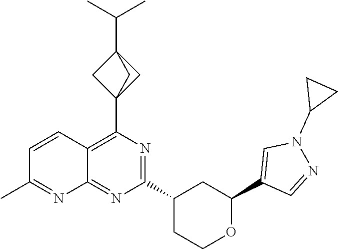 [SMILES: Cc1ccc2c(C34CC(C(C)C)(C3)C4)nc([C@H]3CCO[C@H](c4cnn(C5CC5)c4)C3)nc2n1] |
| I-297 | 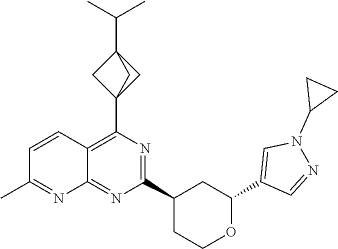 |
| I-298 | 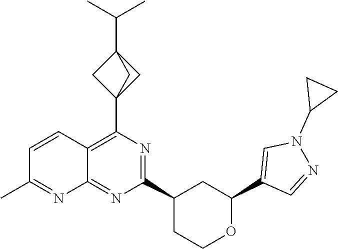 |
| I-299 | 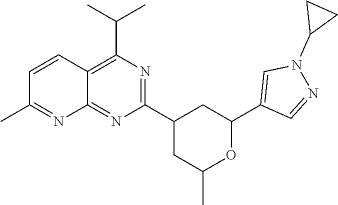 |
| I-300 | 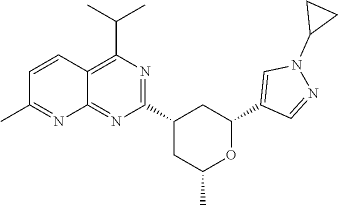 |
| I-301 | 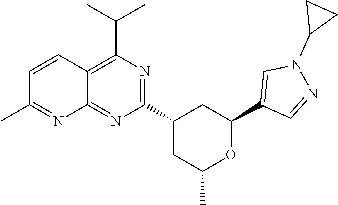 |
| I-302 | 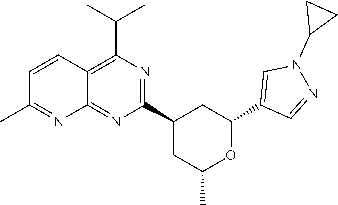 |
| I-303 | 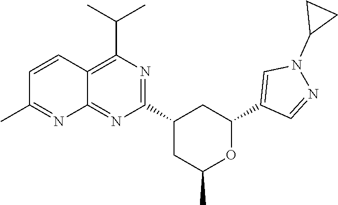 |
| I-304 | 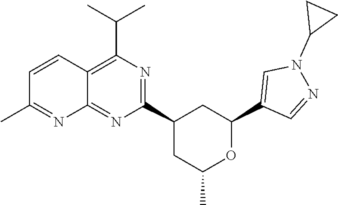 |
| I-305 | 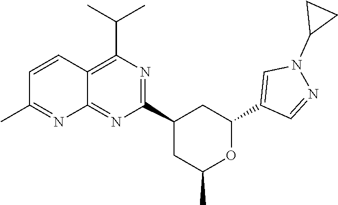 |
| I-306 | 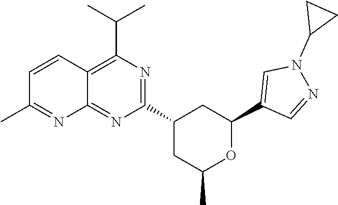 |
| I-307 | 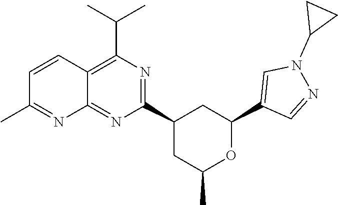 |
| I-308 | 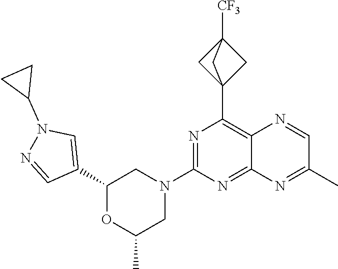 [SMILES: Cc1cnc2c(C34CC(C(F)(F)F)(C3)C4)nc(N3C[C@@H](c4cnn(C5CC5)c4)O[C@@H](C)C3)nc2n1] |
| I-309 | 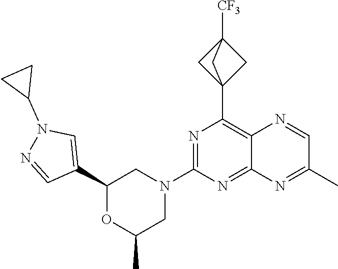 [SMILES: Cc1cnc2c(C34CC(C(F)(F)F)(C3)C4)nc(N3C[C@@H](C)O[C@@H](c4cnn(C5CC5)c4)C3)nc2n1] |
| I-310 | 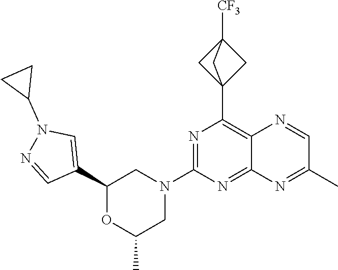 [SMILES: Cc1cnc2c(C34CC(C(F)(F)F)(C3)C4)nc(N3C[C@H](C)O[C@@H](c4cnn(C5CC5)c4)C3)nc2n1] |
| I-311 | 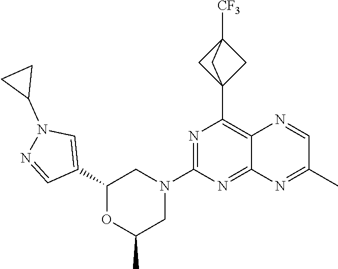 [SMILES: Cc1cnc2c(C34CC(C(F)(F)F)(C3)C4)nc(N3C[C@@H](C)O[C@H](c4cnn(C5CC5)c4)C3)nc2n1] |
| I-312 | 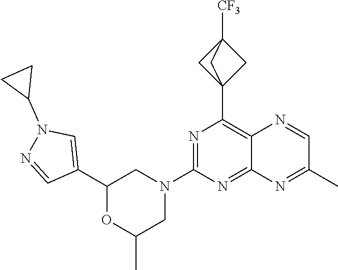 [SMILES: Cc1cnc2c(C34CC(C(F)(F)F)(C3)C4)nc(N3CC(C)OC(c4cnn(C5CC5)c4)C3)nc2n1] |
| I-313 | 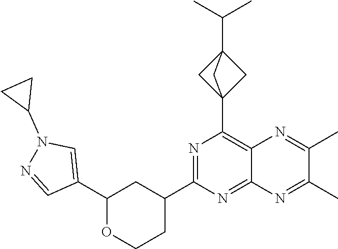 [SMILES: Cc1nc2nc(C3CCOC(c4cnn(C5CC5)c4)C3)nc(C34CC(C(C)C)(C3)C4)c2nc1C] |
| I-314 | 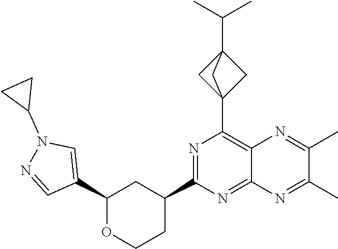 [SMILES: Cc1nc2nc([C@H]3CCO[C@@H](c4cnn(C5CC5)c4)C3)nc(C34CC(C(C)C)(C3)C4)c2nc1C] |
| I-315 | 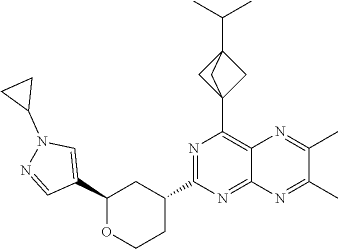 [SMILES: Cc1nc2nc([C@@H]3CCO[C@@H](c4cnn(C5CC5)c4)C3)nc(C34CC(C(C)C)(C3)C4)c2nc1C] |
| I-316 | 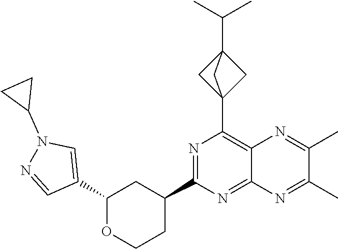 [SMILES: Cc1nc2nc([C@H]3CCO[C@H](c4cnn(C5CC5)c4)C3)nc(C34CC(C(C)C)(C3)C4)c2nc1C] |
| I-317 | 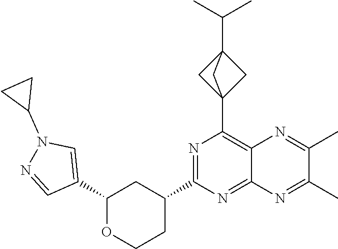 [SMILES: Cc1nc2nc([C@@H]3CCO[C@H](c4cnn(C5CC5)c4)C3)nc(C34CC(C(C)C)(C3)C4)c2nc1C] |
| I-318 | 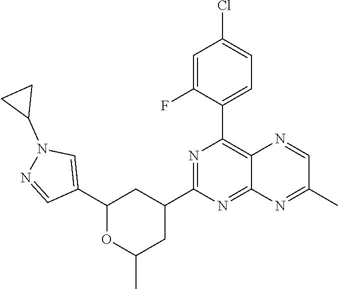 [SMILES: Cc1cnc2c(-c3ccc(Cl)cc3F)nc(C3CC(C)OC(c4cnn(C5CC5)c4)C3)nc2n1] |
| I-319 | 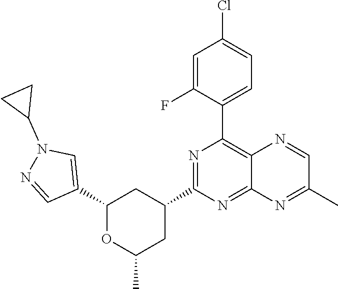 [SMILES: Cc1cnc2c(-c3ccc(Cl)cc3F)nc([C@H]3C[C@@H](c4cnn(C5CC5)c4)O[C@@H](C)C3)nc2n1] |
| I-320 | 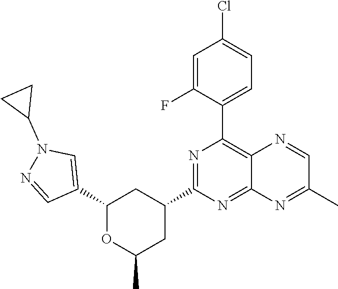 [SMILES: Cc1cnc2c(-c3ccc(Cl)cc3F)nc([C@@H]3C[C@@H](C)O[C@H](c4cnn(C5CC5)c4)C3)nc2n1] |
| I-321 | 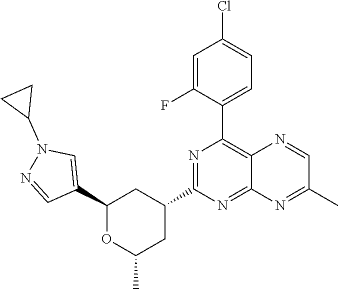 [SMILES: Cc1cnc2c(-c3ccc(Cl)cc3F)nc([C@@H]3C[C@H](C)O[C@@H](c4cnn(C5CC5)c4)C3)nc2n1] |
| I-322 | 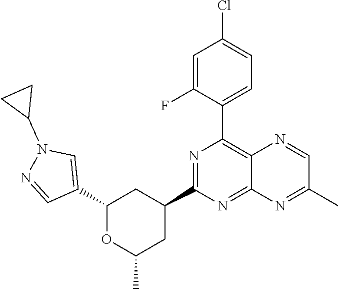 [SMILES: Cc1cnc2c(-c3ccc(Cl)cc3F)nc([C@@H]3C[C@@H](c4cnn(C5CC5)c4)O[C@@H](C)C3)nc2n1] |
| I-323 | 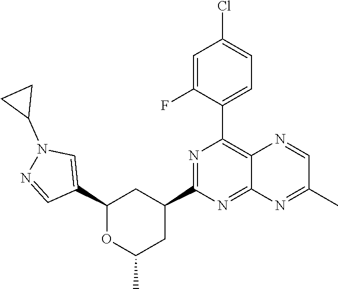 [SMILES: Cc1cnc2c(-c3ccc(Cl)cc3F)nc([C@H]3C[C@H](C)O[C@@H](c4cnn(C5CC5)c4)C3)nc2n1] |
| I-324 | 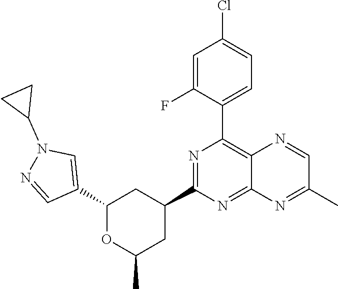 [SMILES: Cc1cnc2c(-c3ccc(Cl)cc3F)nc([C@H]3C[C@@H](C)O[C@H](c4cnn(C5CC5)c4)C3)nc2n1] |
| I-325 | 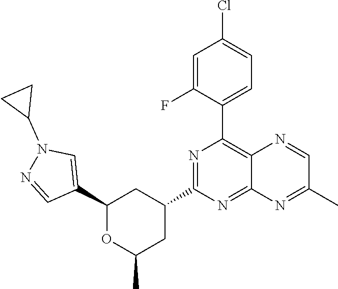 [SMILES: Cc1cnc2c(-c3ccc(Cl)cc3F)nc([C@@H]3C[C@@H](C)O[C@@H](c4cnn(C5CC5)c4)C3)nc2n1] |
| I-326 | 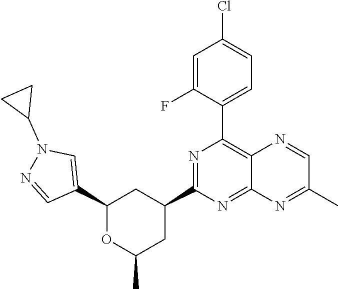 [SMILES: Cc1cnc2c(-c3ccc(Cl)cc3F)nc([C@H]3C[C@@H](C)O[C@@H](c4cnn(C5CC5)c4)C3)nc2n1] |
| I-327 | 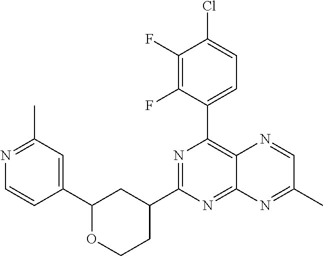 [SMILES: Cc1cc(C2CC(c3nc(-c4ccc(Cl)c(F)c4F)c4ncc(C)nc4n3)CCO2)ccn1] |
| I-328 | 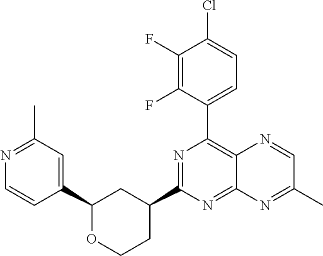 [SMILES: Cc1cc([C@H]2C[C@@H](c3nc(-c4ccc(Cl)c(F)c4F)c4ncc(C)nc4n3)CCO2)ccn1] |
| I-329 | 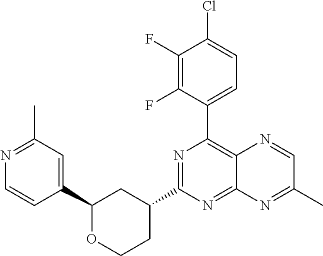 [SMILES: Cc1cc([C@H]2C[C@H](c3nc(-c4ccc(Cl)c(F)c4F)c4ncc(C)nc4n3)CCO2)ccn1] |
| I-330 | 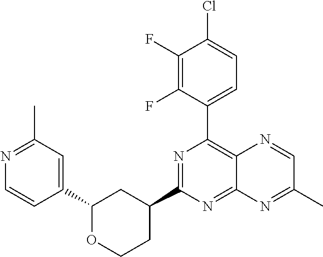 [SMILES: Cc1cc([C@@H]2C[C@@H](c3nc(-c4ccc(Cl)c(F)c4F)c4ncc(C)nc4n3)CCO2)ccn1] |
| I-331 | 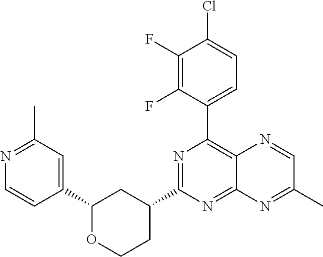 [SMILES: Cc1cc([C@@H]2C[C@H](c3nc(-c4ccc(Cl)c(F)c4F)c4ncc(C)nc4n3)CCO2)ccn1] |
| I-332 | 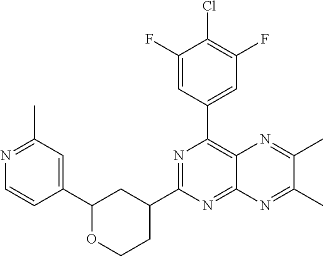 [SMILES: Cc1cc(C2CC(c3nc(-c4cc(F)c(Cl)c(F)c4)c4nc(C)c(C)nc4n3)CCO2)ccn1] |
| I-333 | 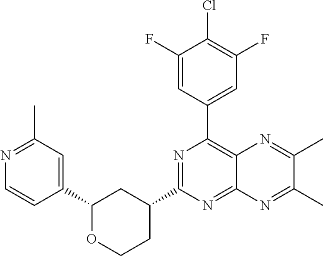 [SMILES: Cc1cc([C@@H]2C[C@H](c3nc(-c4cc(F)c(Cl)c(F)c4)c4nc(C)c(C)nc4n3)CCO2)ccn1] |
| I-334 | 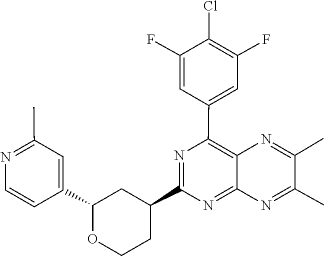 [SMILES: Cc1cc([C@@H]2C[C@@H](c3nc(-c4cc(F)c(Cl)c(F)c4)c4nc(C)c(C)nc4n3)CCO2)ccn1] |
| I-335 | 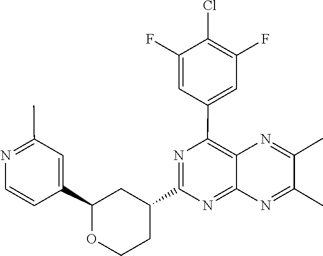 [SMILES: Cc1cc([C@H]2C[C@H](c3nc(-c4cc(F)c(Cl)c(F)c4)c4nc(C)c(C)nc4n3)CCO2)ccn1] |
| I-336 | 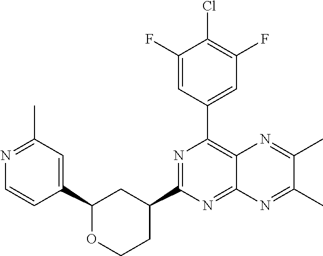 [SMILES: Cc1cc([C@H]2C[C@@H](c3nc(-c4cc(F)c(Cl)c(F)c4)c4nc(C)c(C)nc4n3)CCO2)ccn1] |
| I-337 | 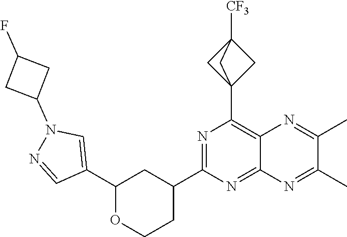 [SMILES: Cc1nc2nc(C3CCOC(c4cnn(C5CC(F)C5)c4)C3)nc(C34CC(C(F)(F)F)(C3)C4)c2nc1C] |
| I-338 | 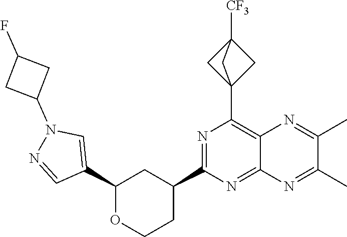 [SMILES: Cc1nc2nc([C@H]3CCO[C@@H](c4cnn(C5CC(F)C5)c4)C3)nc(C34CC(C(F)(F)F)(C3)C4)c2nc1C] |
| I-339 | 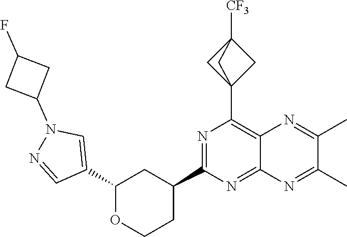 [SMILES: Cc1nc2nc([C@H]3CCO[C@H](c4cnn(C5CC(F)C5)c4)C3)nc(C34CC(C(F)(F)F)(C3)C4)c2nc1C] |
| I-340 | 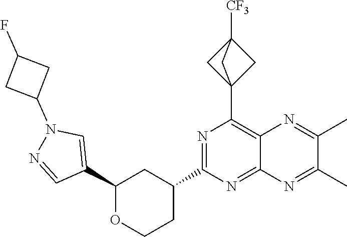 [SMILES: Cc1nc2nc([C@@H]3CCO[C@@H](c4cnn(C5CC(F)C5)c4)C3)nc(C34CC(C(F)(F)F)(C3)C4)c2nc1C] |
| I-341 | 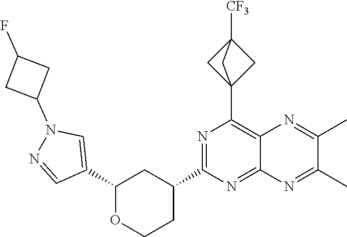 [SMILES: Cc1nc2nc([C@@H]3CCO[C@H](c4cnn(C5CC(F)C5)c4)C3)nc(C34CC(C(F)(F)F)(C3)C4)c2nc1C] |
| I-342 | 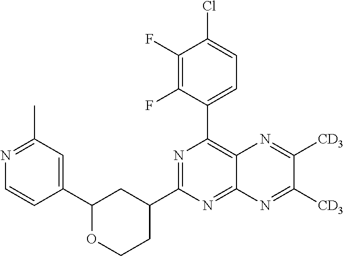 [SMILES: [2H]C([2H])([2H])c1nc2nc(C3CCOC(c4ccnc(C)c4)C3)nc(-c3ccc(Cl)c(F)c3F)c2nc1C([2H])([2H])[2H]] |
| I-343 | 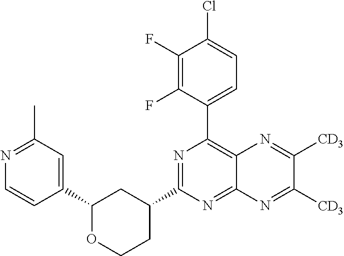 [SMILES: [2H]C([2H])([2H])c1nc2nc([C@@H]3CCO[C@H](c4ccnc(C)c4)C3)nc(-c3ccc(Cl)c(F)c3F)c2nc1C([2H])([2H])[2H]] |
| I-344 | 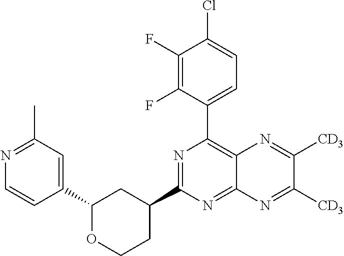 [SMILES: [2H]C([2H])([2H])c1nc2nc([C@H]3CCO[C@H](c4ccnc(C)c4)C3)nc(-c3ccc(Cl)c(F)c3F)c2nc1C([2H])([2H])[2H]] |
| I-345 | 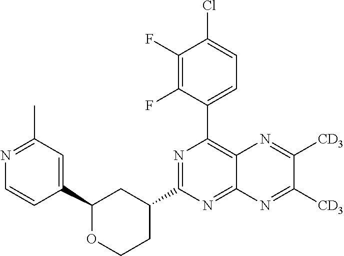 [SMILES: [2H]C([2H])([2H])c1nc2nc([C@@H]3CCO[C@@H](c4ccnc(C)c4)C3)nc(-c3ccc(Cl)c(F)c3F)c2nc1C([2H])([2H])[2H]] |
| I-346 | 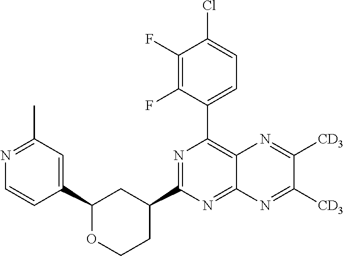 [SMILES: [2H]C([2H])([2H])c1nc2nc([C@H]3CCO[C@@H](c4ccnc(C)c4)C3)nc(-c3ccc(Cl)c(F)c3F)c2nc1C([2H])([2H])[2H]] |
| I-347 | 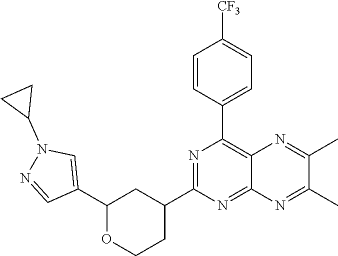 [SMILES: Cc1nc2nc(C3CCOC(c4cnn(C5CC5)c4)C3)nc(-c3ccc(C(F)(F)F)cc3)c2nc1C] |
| I-348 | 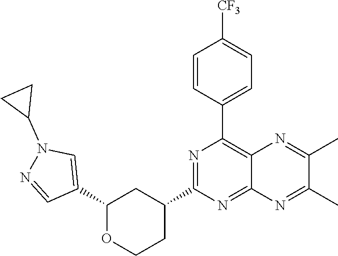 [SMILES: Cc1nc2nc([C@@H]3CCO[C@H](c4cnn(C5CC5)c4)C3)nc(-c3ccc(C(F)(F)F)cc3)c2nc1C] |
| I-349 | 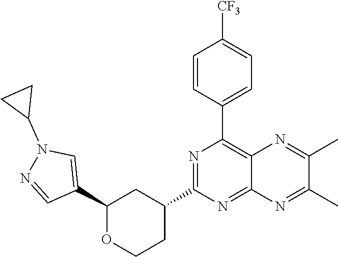 [SMILES: Cc1nc2nc([C@@H]3CCO[C@@H](c4cnn(C5CC5)c4)C3)nc(-c3ccc(C(F)(F)F)cc3)c2nc1C] |
| I-350 | 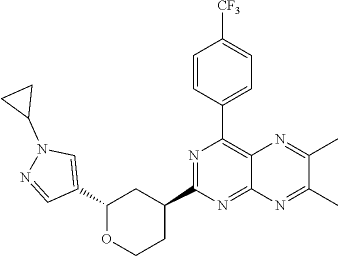 [SMILES: Cc1nc2nc([C@H]3CCO[C@H](c4cnn(C5CC5)c4)C3)nc(-c3ccc(C(F)(F)F)cc3)c2nc1C] |
| I-351 | 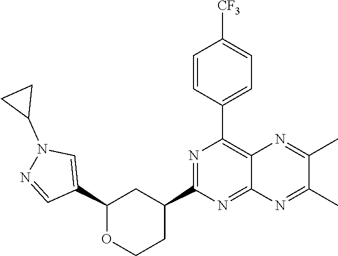 [SMILES: Cc1nc2nc([C@H]3CCO[C@@H](c4cnn(C5CC5)c4)C3)nc(-c3ccc(C(F)(F)F)cc3)c2nc1C] |
| I-352 | 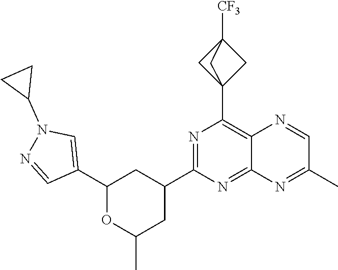 [SMILES: Cc1cnc2c(C34CC(C(F)(F)F)(C3)C4)nc(C3CC(C)OC(c4cnn(C5CC5)c4)C3)nc2n1] |
| I-353 | 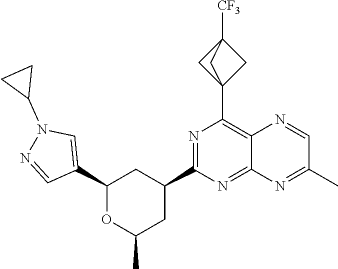 [SMILES: Cc1cnc2c(C34CC(C(F)(F)F)(C3)C4)nc([C@H]3C[C@@H](C)O[C@@H](c4cnn(C5CC5)c4)C3)nc2n1] |
| I-354 | 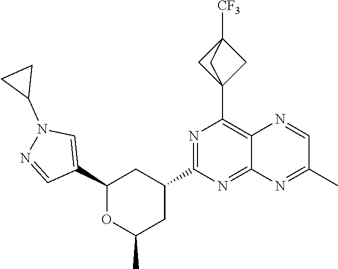 [SMILES: Cc1cnc2c(C34CC(C(F)(F)F)(C3)C4)nc([C@@H]3C[C@@H](C)O[C@@H](c4cnn(C5CC5)c4)C3)nc2n1] |
| I-355 | 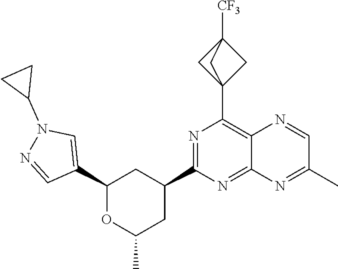 [SMILES: Cc1cnc2c(C34CC(C(F)(F)F)(C3)C4)nc([C@H]3C[C@H](C)O[C@@H](c4cnn(C5CC5)c4)C3)nc2n1] |
| I-356 | 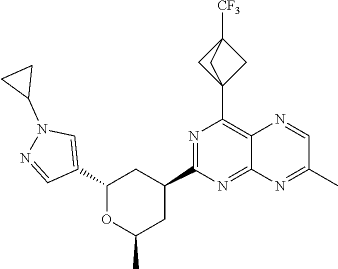 [SMILES: Cc1cnc2c(C34CC(C(F)(F)F)(C3)C4)nc([C@H]3C[C@@H](C)O[C@H](c4cnn(C5CC5)c4)C3)nc2n1] |
| I-357 | 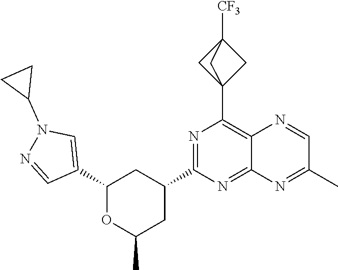 [SMILES: Cc1cnc2c(C34CC(C(F)(F)F)(C3)C4)nc([C@@H]3C[C@@H](C)O[C@H](c4cnn(C5CC5)c4)C3)nc2n1] |
| I-358 | 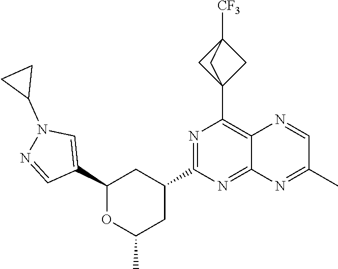 [SMILES: Cc1cnc2c(C34CC(C(F)(F)F)(C3)C4)nc([C@@H]3C[C@H](C)O[C@@H](c4cnn(C5CC5)c4)C3)nc2n1] |
| I-359 | 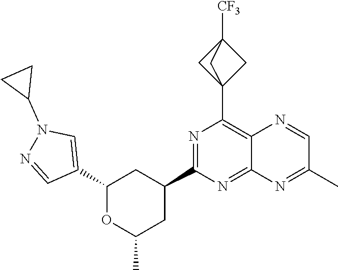 [SMILES: Cc1cnc2c(C34CC(C(F)(F)F)(C3)C4)nc([C@@H]3C[C@@H](c4cnn(C5CC5)c4)O[C@@H](C)C3)nc2n1] |
| I-360 | 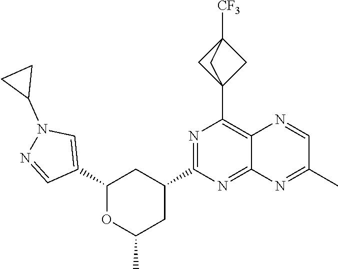 [SMILES: Cc1cnc2c(C34CC(C(F)(F)F)(C3)C4)nc([C@H]3C[C@@H](c4cnn(C5CC5)c4)O[C@@H](C)C3)nc2n1] |
| I-361 | 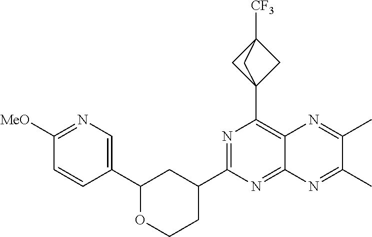 [SMILES: COc1ccc(C2CC(c3nc(C45CC(C(F)(F)F)(C4)C5)c4nc(C)c(C)nc4n3)CCO2)cn1] |
| I-362 | 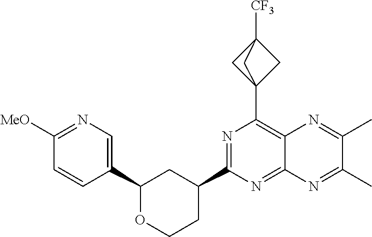 [SMILES: COc1ccc([C@H]2C[C@@H](c3nc(C45CC(C(F)(F)F)(C4)C5)c4nc(C)c(C)nc4n3)CCO2)cn1] |
| I-363 | 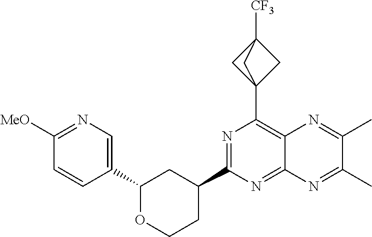 [SMILES: COc1ccc([C@@H]2C[C@@H](c3nc(C45CC(C(F)(F)F)(C4)C5)c4nc(C)c(C)nc4n3)CCO2)cn1] |
| I-364 | 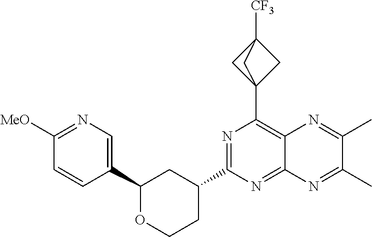 [SMILES: COc1ccc([C@H]2C[C@H](c3nc(C45CC(C(F)(F)F)(C4)C5)c4nc(C)c(C)nc4n3)CCO2)cn1] |
| I-365 | 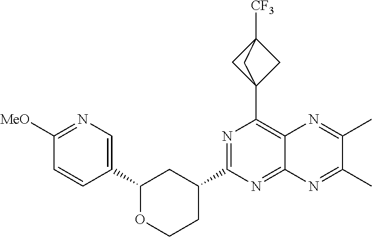 [SMILES: COc1ccc([C@@H]2C[C@H](c3nc(C45CC(C(F)(F)F)(C4)C5)c4nc(C)c(C)nc4n3)CCO2)cn1] |
| I-366 | 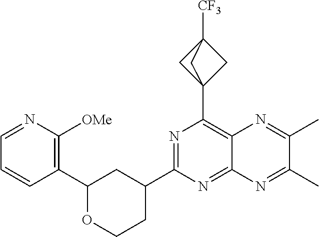 [SMILES: COc1ncccc1C1CC(c2nc(C34CC(C(F)(F)F)(C3)C4)c3nc(C)c(C)nc3n2)CCO1] |
| I-367 | 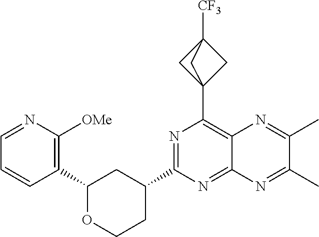 [SMILES: COc1ncccc1[C@@H]1C[C@H](c2nc(C34CC(C(F)(F)F)(C3)C4)c3nc(C)c(C)nc3n2)CCO1] |
| I-368 | 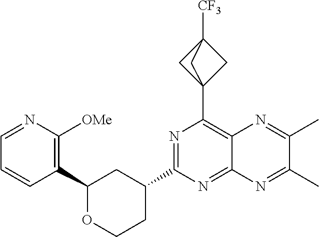 [SMILES: COc1ncccc1[C@H]1C[C@H](c2nc(C34CC(C(F)(F)F)(C3)C4)c3nc(C)c(C)nc3n2)CCO1] |
| I-369 | 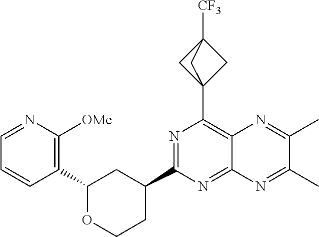 [SMILES: COc1ncccc1[C@@H]1C[C@@H](c2nc(C34CC(C(F)(F)F)(C3)C4)c3nc(C)c(C)nc3n2)CCO1] |
| I-370 | 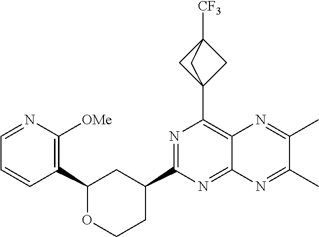 [SMILES: COc1ncccc1[C@H]1C[C@@H](c2nc(C34CC(C(F)(F)F)(C3)C4)c3nc(C)c(C)nc3n2)CCO1] |
| I-371 | 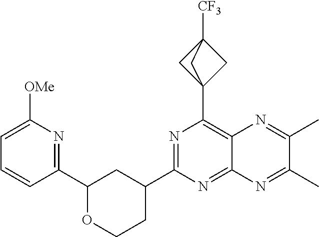 [SMILES: COc1cccc(C2CC(c3nc(C45CC(C(F)(F)F)(C4)C5)c4nc(C)c(C)nc4n3)CCO2)n1] |
| I-372 | 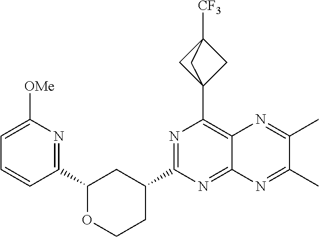 [SMILES: COc1cccc([C@@H]2C[C@H](c3nc(C45CC(C(F)(F)F)(C4)C5)c4nc(C)c(C)nc4n3)CCO2)n1] |
| I-373 | 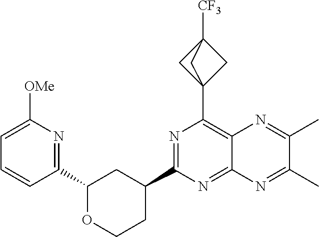 [SMILES: COc1cccc([C@@H]2C[C@@H](c3nc(C45CC(C(F)(F)F)(C4)C5)c4nc(C)c(C)nc4n3)CCO2)n1] |
| I-374 | 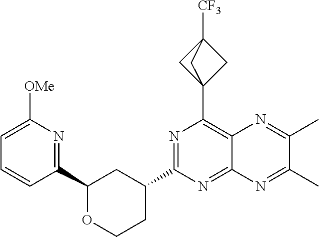 [SMILES: COc1cccc([C@H]2C[C@H](c3nc(C45CC(C(F)(F)F)(C4)C5)c4nc(C)c(C)nc4n3)CCO2)n1] |
| I-375 | 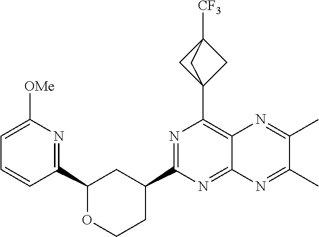 [SMILES: COc1cccc([C@H]2C[C@@H](c3nc(C45CC(C(F)(F)F)(C4)C5)c4nc(C)c(C)nc4n3)CCO2)n1] |
| I-376 | 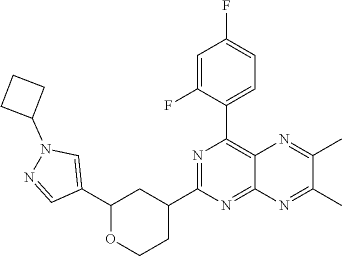 [SMILES: Cc1nc2nc(C3CCOC(c4cnn(C5CCC5)c4)C3)nc(-c3ccc(F)cc3F)c2nc1C] |
| I-377 | 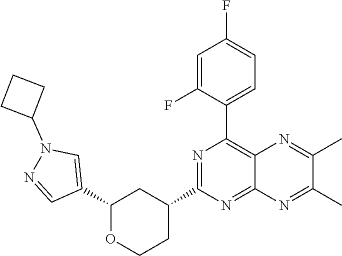 [SMILES: Cc1nc2nc([C@@H]3CCO[C@H](c4cnn(C5CCC5)c4)C3)nc(-c3ccc(F)cc3F)c2nc1C] |
| I-378 | 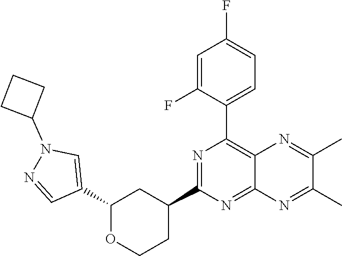 [SMILES: Cc1nc2nc([C@H]3CCO[C@H](c4cnn(C5CCC5)c4)C3)nc(-c3ccc(F)cc3F)c2nc1C] |
| I-379 | 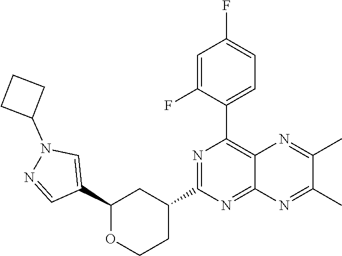 [SMILES: Cc1nc2nc([C@@H]3CCO[C@@H](c4cnn(C5CCC5)c4)C3)nc(-c3ccc(F)cc3F)c2nc1C] |
| I-380 | 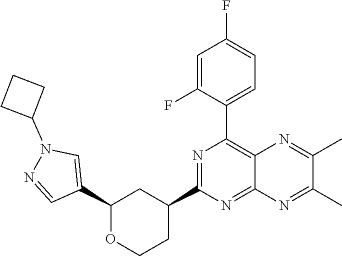 [SMILES: Cc1nc2nc([C@H]3CCO[C@@H](c4cnn(C5CCC5)c4)C3)nc(-c3ccc(F)cc3F)c2nc1C] |
| I-381 | 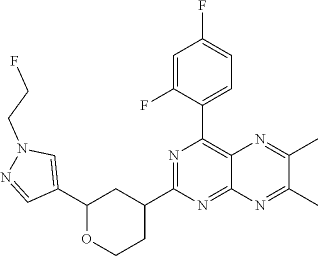 [SMILES: Cc1nc2nc(C3CCOC(c4cnn(CCF)c4)C3)nc(-c3ccc(F)cc3F)c2nc1C] |
| I-382 | 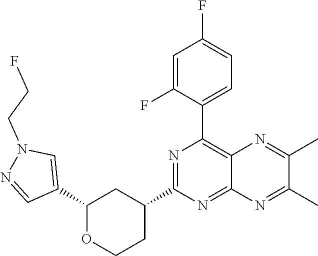 [SMILES: Cc1nc2nc([C@@H]3CCO[C@H](c4cnn(CCF)c4)C3)nc(-c3ccc(F)cc3F)c2nc1C] |
| I-383 | 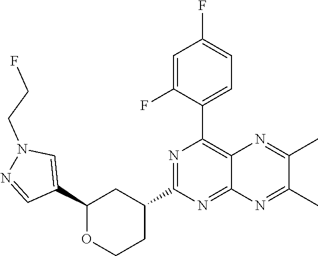 [SMILES: Cc1nc2nc([C@@H]3CCO[C@@H](c4cnn(CCF)c4)C3)nc(-c3ccc(F)cc3F)c2nc1C] |
| I-384 | 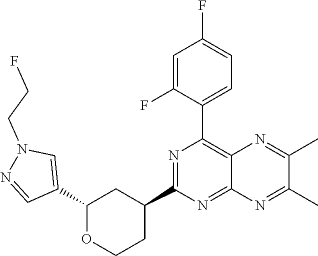 [SMILES: Cc1nc2nc([C@H]3CCO[C@H](c4cnn(CCF)c4)C3)nc(-c3ccc(F)cc3F)c2nc1C] |
| I-385 | 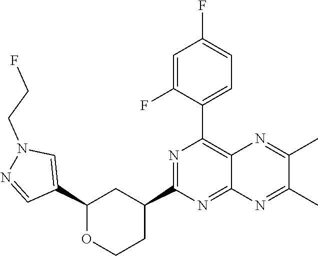 [SMILES: Cc1nc2nc([C@H]3CCO[C@@H](c4cnn(CCF)c4)C3)nc(-c3ccc(F)cc3F)c2nc1C] |
| I-386 | 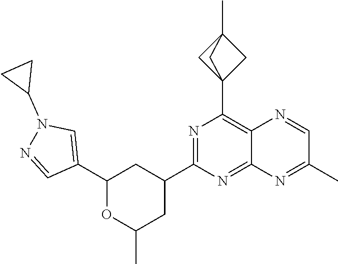 [SMILES: Cc1cnc2c(C34CC(C)(C3)C4)nc(C3CC(C)OC(c4cnn(C5CC5)c4)C3)nc2n1] |
| I-387 | 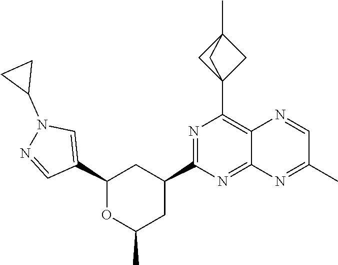 [SMILES: Cc1cnc2c(C34CC(C)(C3)C4)nc([C@H]3C[C@@H](C)O[C@@H](c4cnn(C5CC5)c4)C3)nc2n1] |
| I-388 | 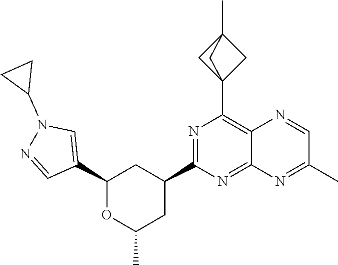 [SMILES: Cc1cnc2c(C34CC(C)(C3)C4)nc([C@H]3C[C@H](C)O[C@@H](c4cnn(C5CC5)c4)C3)nc2n1] |
| I-389 | 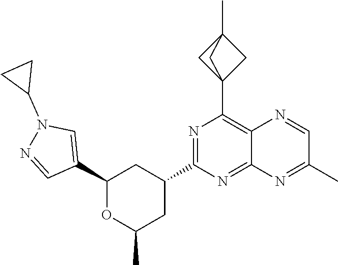 [SMILES: Cc1cnc2c(C34CC(C)(C3)C4)nc([C@@H]3C[C@@H](C)O[C@@H](c4cnn(C5CC5)c4)C3)nc2n1] |
| I-390 | 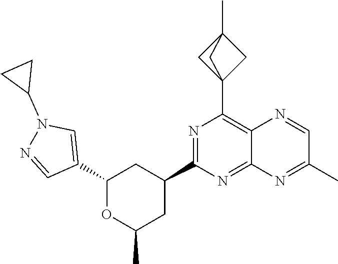 [SMILES: Cc1cnc2c(C34CC(C)(C3)C4)nc([C@H]3C[C@@H](C)O[C@H](c4cnn(C5CC5)c4)C3)nc2n1] |
| I-391 | 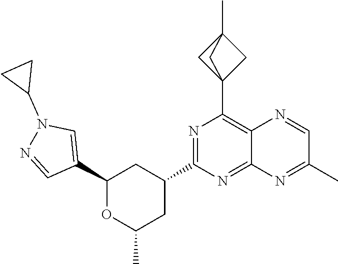 [SMILES: Cc1cnc2c(C34CC(C)(C3)C4)nc([C@@H]3C[C@H](C)O[C@@H](c4cnn(C5CC5)c4)C3)nc2n1] |
| I-392 | 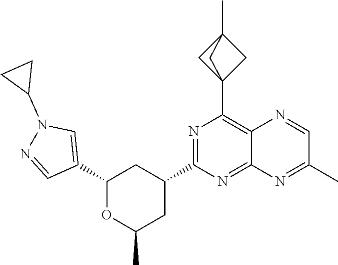 [SMILES: Cc1cnc2c(C34CC(C)(C3)C4)nc([C@@H]3C[C@@H](C)O[C@H](c4cnn(C5CC5)c4)C3)nc2n1] |
| I-393 | 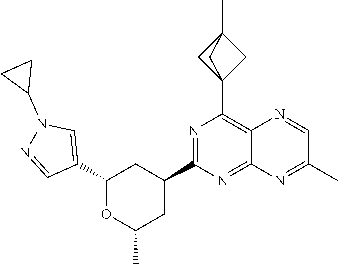 [SMILES: Cc1cnc2c(C34CC(C)(C3)C4)nc([C@@H]3C[C@@H](c4cnn(C5CC5)c4)O[C@@H](C)C3)nc2n1] |
| I-394 | 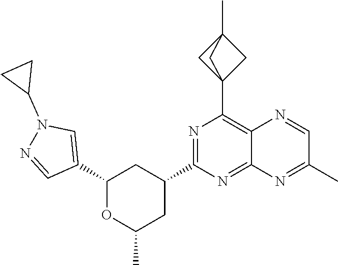 [SMILES: Cc1cnc2c(C34CC(C)(C3)C4)nc([C@H]3C[C@@H](c4cnn(C5CC5)c4)O[C@@H](C)C3)nc2n1] |
| I-395 | 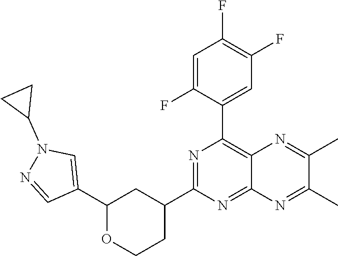 [SMILES: Cc1nc2nc(C3CCOC(c4cnn(C5CC5)c4)C3)nc(-c3cc(F)c(F)cc3F)c2nc1C] |
| I-396 | 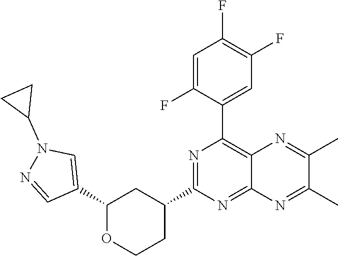 [SMILES: Cc1nc2nc([C@@H]3CCO[C@H](c4cnn(C5CC5)c4)C3)nc(-c3cc(F)c(F)cc3F)c2nc1C] |
| I-397 | 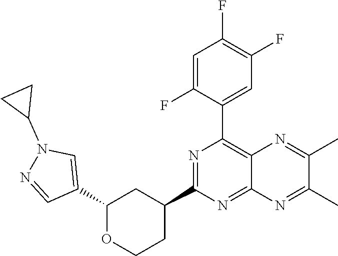 [SMILES: Cc1nc2nc([C@H]3CCO[C@H](c4cnn(C5CC5)c4)C3)nc(-c3cc(F)c(F)cc3F)c2nc1C] |
| I-398 | 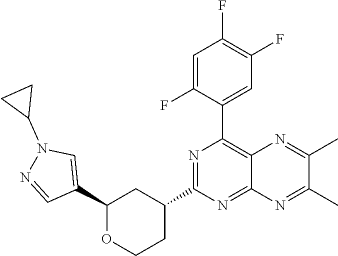 [SMILES: Cc1nc2nc([C@@H]3CCO[C@@H](c4cnn(C5CC5)c4)C3)nc(-c3cc(F)c(F)cc3F)c2nc1C] |
| I-399 | 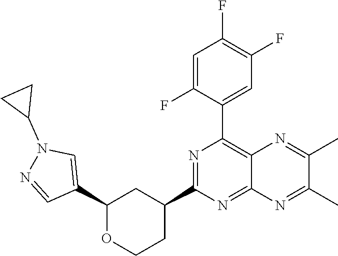 [SMILES: Cc1nc2nc([C@H]3CCO[C@@H](c4cnn(C5CC5)c4)C3)nc(-c3cc(F)c(F)cc3F)c2nc1C] |
| I-400 | 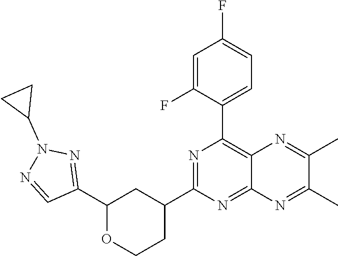 [SMILES: Cc1nc2nc(C3CCOC(c4cnn(C5CC5)n4)C3)nc(-c3ccc(F)cc3F)c2nc1C] |
| I-401 | 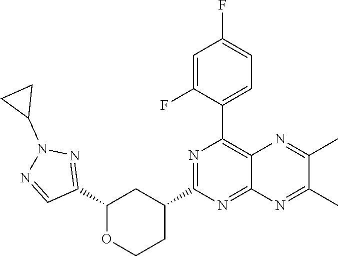 [SMILES: Cc1nc2nc([C@@H]3CCO[C@H](c4cnn(C5CC5)n4)C3)nc(-c3ccc(F)cc3F)c2nc1C] |
| I-402 | 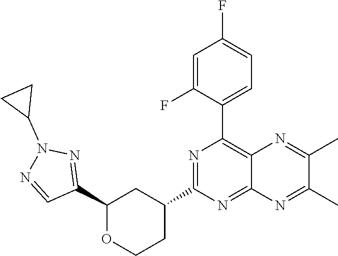 [SMILES: Cc1nc2nc([C@@H]3CCO[C@@H](c4cnn(C5CC5)n4)C3)nc(-c3ccc(F)cc3F)c2nc1C] |
| I-403 | 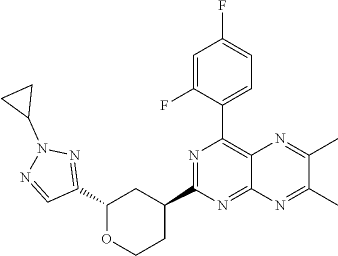 [SMILES: Cc1nc2nc([C@H]3CCO[C@H](c4cnn(C5CC5)n4)C3)nc(-c3ccc(F)cc3F)c2nc1C] |
| I-404 | 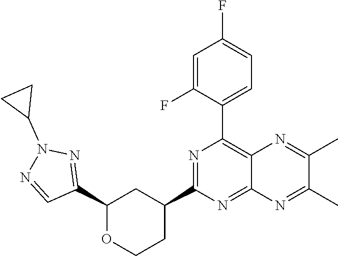 [SMILES: Cc1nc2nc([C@H]3CCO[C@@H](c4cnn(C5CC5)n4)C3)nc(-c3ccc(F)cc3F)c2nc1C] |
| I-405 | 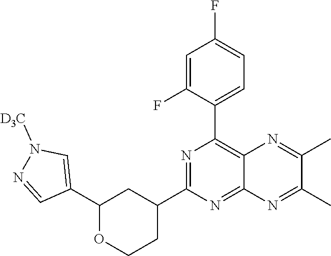 [SMILES: [2H]C([2H])([2H])n1cc(C2CC(c3nc(-c4ccc(F)cc4F)c4nc(C)c(C)nc4n3)CCO2)cn1] |
| I-406 | 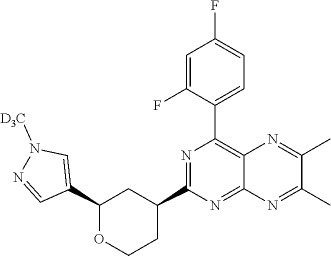 [SMILES: [2H]C([2H])([2H])n1cc([C@H]2C[C@@H](c3nc(-c4ccc(F)cc4F)c4nc(C)c(C)nc4n3)CCO2)cn1] |
| I-407 | 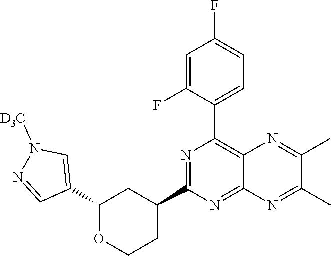 [SMILES: [2H]C([2H])([2H])n1cc([C@@H]2C[C@@H](c3nc(-c4ccc(F)cc4F)c4nc(C)c(C)nc4n3)CCO2)cn1] |
| I-408 | 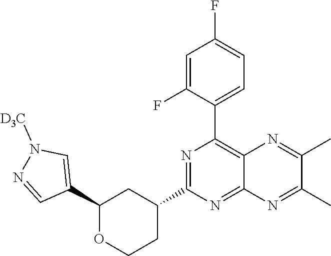 [SMILES: [2H]C([2H])([2H])n1cc([C@H]2C[C@H](c3nc(-c4ccc(F)cc4F)c4nc(C)c(C)nc4n3)CCO2)cn1] |
| I-409 | 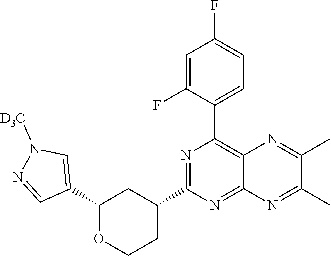 [SMILES: [2H]C([2H])([2H])n1cc([C@@H]2C[C@H](c3nc(-c4ccc(F)cc4F)c4nc(C)c(C)nc4n3)CCO2)cn1] |
| I-410 | 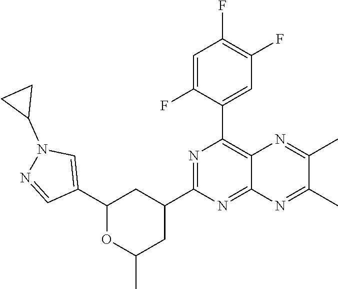 [SMILES: Cc1nc2nc(C3CC(C)OC(c4cnn(C5CC5)c4)C3)nc(-c3cc(F)c(F)cc3F)c2nc1C] |
| I-411 | 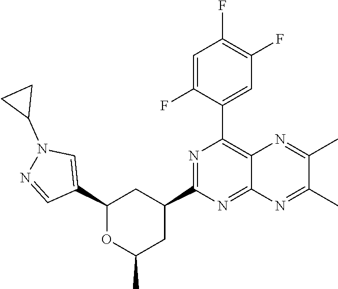 [SMILES: Cc1nc2nc([C@H]3C[C@@H](C)O[C@@H](c4cnn(C5CC5)c4)C3)nc(-c3cc(F)c(F)cc3F)c2nc1C] |
| I-412 | 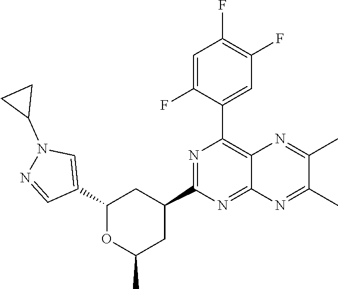 [SMILES: Cc1nc2nc([C@H]3C[C@@H](C)O[C@H](c4cnn(C5CC5)c4)C3)nc(-c3cc(F)c(F)cc3F)c2nc1C] |
| I-413 | 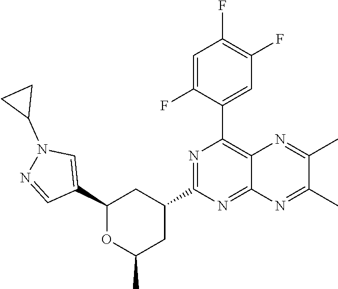 [SMILES: Cc1nc2nc([C@@H]3C[C@@H](C)O[C@@H](c4cnn(C5CC5)c4)C3)nc(-c3cc(F)c(F)cc3F)c2nc1C] |
| I-414 | 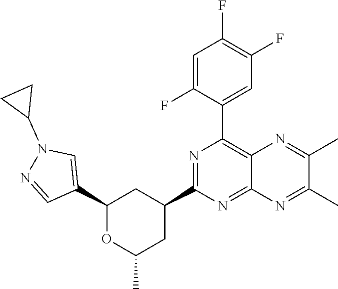 [SMILES: Cc1nc2nc([C@H]3C[C@H](C)O[C@@H](c4cnn(C5CC5)c4)C3)nc(-c3cc(F)c(F)cc3F)c2nc1C] |
| I-415 | 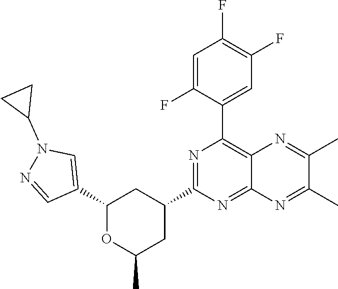 [SMILES: Cc1nc2nc([C@@H]3C[C@@H](C)O[C@H](c4cnn(C5CC5)c4)C3)nc(-c3cc(F)c(F)cc3F)c2nc1C] |
| I-416 | 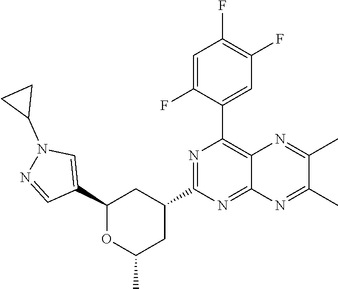 [SMILES: Cc1nc2nc([C@@H]3C[C@H](C)O[C@@H](c4cnn(C5CC5)c4)C3)nc(-c3cc(F)c(F)cc3F)c2nc1C] |
| I-417 | 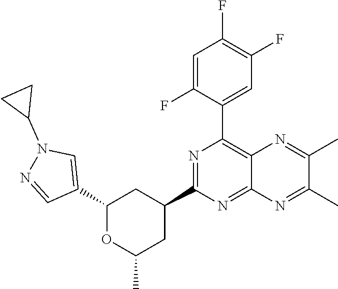 [SMILES: Cc1nc2nc([C@@H]3C[C@@H](c4cnn(C5CC5)c4)O[C@@H](C)C3)nc(-c3cc(F)c(F)cc3F)c2nc1C] |
| I-418 | 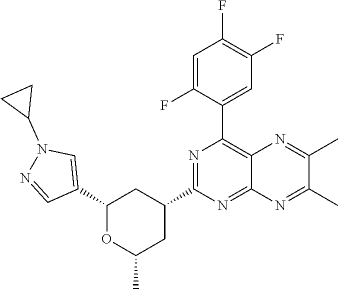 [SMILES: Cc1nc2nc([C@H]3C[C@@H](c4cnn(C5CC5)c4)O[C@@H](C)C3)nc(-c3cc(F)c(F)cc3F)c2nc1C] |
| I-419 | 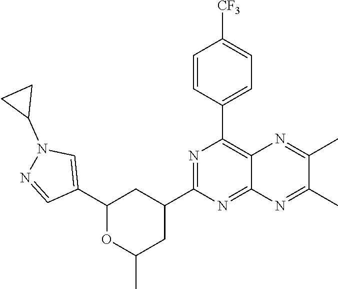 [SMILES: Cc1nc2nc(C3CC(C)OC(c4cnn(C5CC5)c4)C3)nc(-c3ccc(C(F)(F)F)cc3)c2nc1C] |
| I-420 | 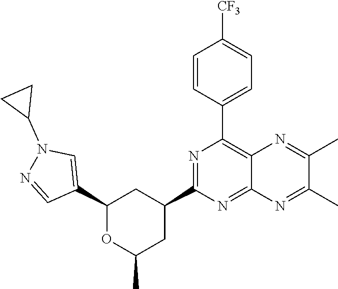 [SMILES: Cc1nc2nc([C@H]3C[C@@H](C)O[C@@H](c4cnn(C5CC5)c4)C3)nc(-c3ccc(C(F)(F)F)cc3)c2nc1C] |
| I-421 | 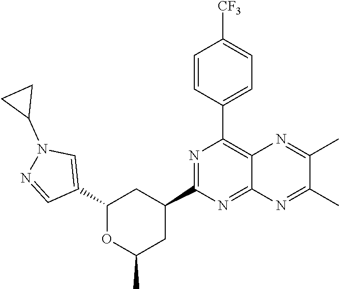 [SMILES: Cc1nc2nc([C@H]3C[C@@H](C)O[C@H](c4cnn(C5CC5)c4)C3)nc(-c3ccc(C(F)(F)F)cc3)c2nc1C] |
| I-422 | 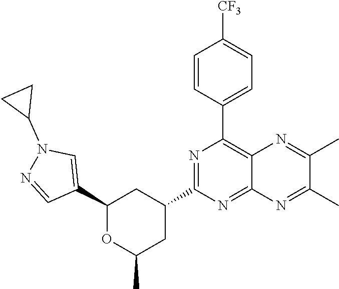 [SMILES: Cc1nc2nc([C@@H]3C[C@@H](C)O[C@@H](c4cnn(C5CC5)c4)C3)nc(-c3ccc(C(F)(F)F)cc3)c2nc1C] |
| I-423 | 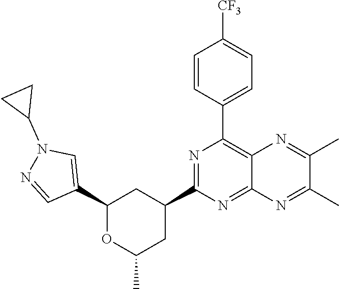 [SMILES: Cc1nc2nc([C@H]3C[C@H](C)O[C@@H](c4cnn(C5CC5)c4)C3)nc(-c3ccc(C(F)(F)F)cc3)c2nc1C] |
| I-424 | 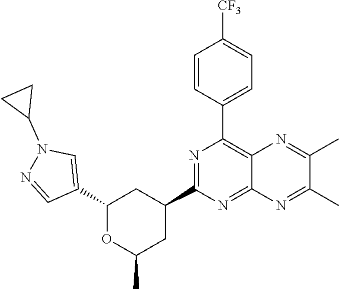 [SMILES: Cc1nc2nc([C@H]3C[C@@H](C)O[C@H](c4cnn(C5CC5)c4)C3)nc(-c3ccc(C(F)(F)F)cc3)c2nc1C] |
| I-425 | 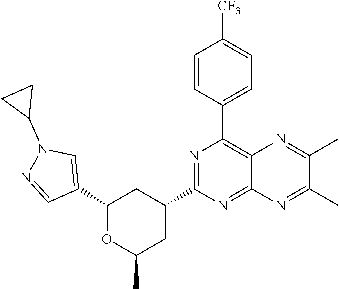 [SMILES: Cc1nc2nc([C@@H]3C[C@@H](C)O[C@H](c4cnn(C5CC5)c4)C3)nc(-c3ccc(C(F)(F)F)cc3)c2nc1C] |
| I-426 | 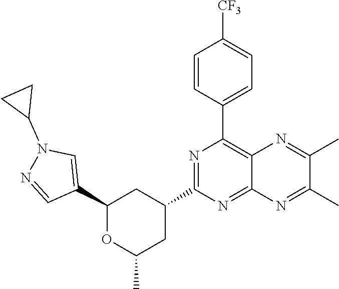 [SMILES: Cc1nc2nc([C@@H]3C[C@H](C)O[C@@H](c4cnn(C5CC5)c4)C3)nc(-c3ccc(C(F)(F)F)cc3)c2nc1C] |
| I-427 | 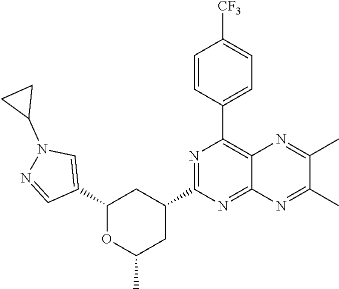 [SMILES: Cc1nc2nc([C@H]3C[C@@H](c4cnn(C5CC5)c4)O[C@@H](C)C3)nc(-c3ccc(C(F)(F)F)cc3)c2nc1C] |
| I-428 | 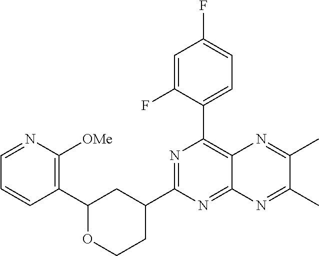 [SMILES: COc1ncccc1C1CC(c2nc(-c3ccc(F)cc3F)c3nc(C)c(C)nc3n2)CCO1] |
| I-429 | 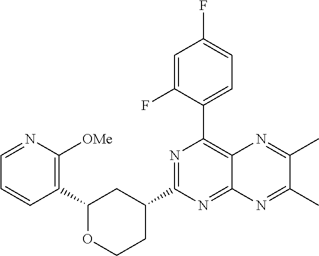 [SMILES: COc1ncccc1[C@@H]1C[C@H](c2nc(-c3ccc(F)cc3F)c3nc(C)c(C)nc3n2)CCO1] |
| I-430 | 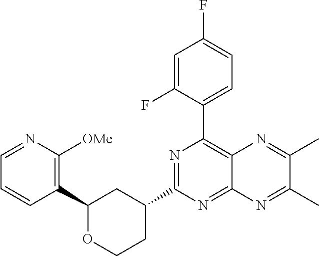 [SMILES: COc1ncccc1[C@H]1C[C@H](c2nc(-c3ccc(F)cc3F)c3nc(C)c(C)nc3n2)CCO1] |
| I-431 | 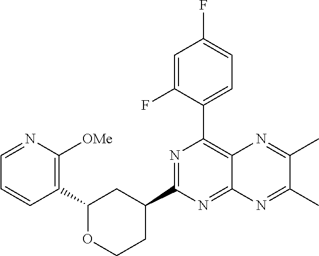 [SMILES: COc1ncccc1[C@@H]1C[C@@H](c2nc(-c3ccc(F)cc3F)c3nc(C)c(C)nc3n2)CCO1] |
| I-432 | 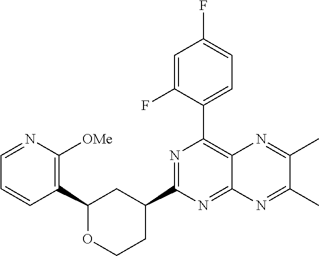 [SMILES: COc1ncccc1[C@H]1C[C@@H](c2nc(-c3ccc(F)cc3F)c3nc(C)c(C)nc3n2)CCO1] |
| I-433 | 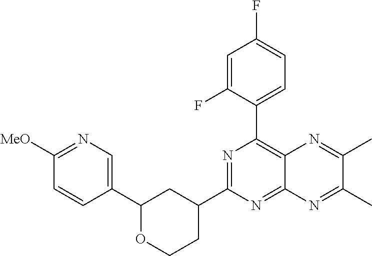 [SMILES: COc1ccc(C2CC(c3nc(-c4ccc(F)cc4F)c4nc(C)c(C)nc4n3)CCO2)cn1] |
| I-434 | 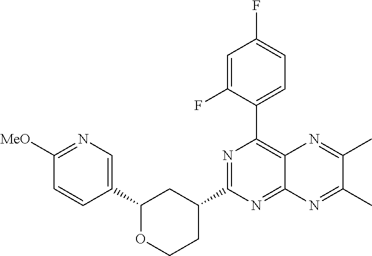 [SMILES: COc1ccc([C@@H]2C[C@H](c3nc(-c4ccc(F)cc4F)c4nc(C)c(C)nc4n3)CCO2)cn1] |
| I-435 | 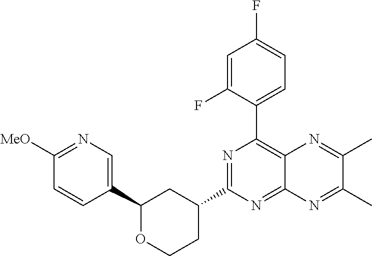 [SMILES: COc1ccc([C@H]2C[C@H](c3nc(-c4ccc(F)cc4F)c4nc(C)c(C)nc4n3)CCO2)cn1] |
| I-436 | 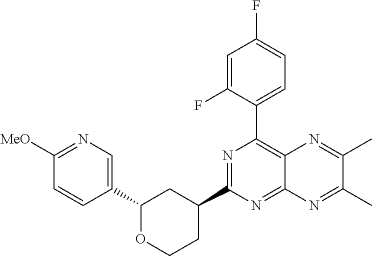 [SMILES: COc1ccc([C@@H]2C[C@@H](c3nc(-c4ccc(F)cc4F)c4nc(C)c(C)nc4n3)CCO2)cn1] |
| I-437 | 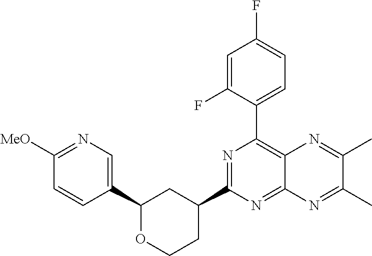 [SMILES: COc1ccc([C@H]2C[C@@H](c3nc(-c4ccc(F)cc4F)c4nc(C)c(C)nc4n3)CCO2)cn1] |
| I-438 | 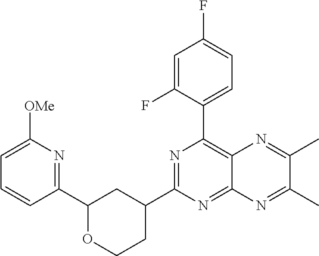 [SMILES: COc1cccc(C2CC(c3nc(-c4ccc(F)cc4F)c4nc(C)c(C)nc4n3)CCO2)n1] |
| I-439 | 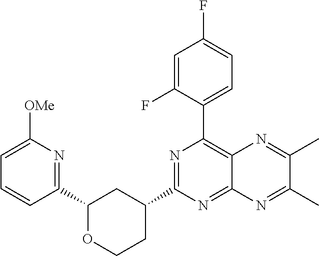 [SMILES: COc1cccc([C@@H]2C[C@H](c3nc(-c4ccc(F)cc4F)c4nc(C)c(C)nc4n3)CCO2)n1] |
| I-440 | 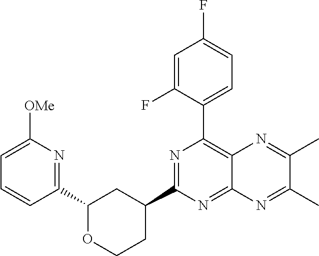 [SMILES: COc1cccc([C@@H]2C[C@@H](c3nc(-c4ccc(F)cc4F)c4nc(C)c(C)nc4n3)CCO2)n1] |
| I-441 | 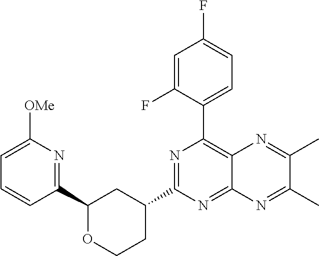 [SMILES: COc1cccc([C@H]2C[C@H](c3nc(-c4ccc(F)cc4F)c4nc(C)c(C)nc4n3)CCO2)n1] |
| I-442 | 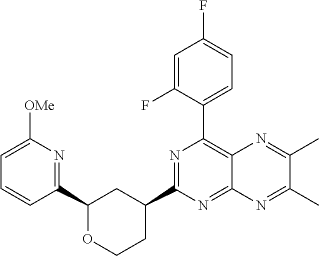 [SMILES: COc1cccc([C@H]2C[C@@H](c3nc(-c4ccc(F)cc4F)c4nc(C)c(C)nc4n3)CCO2)n1] |
| I-443 | 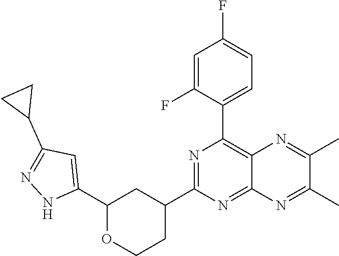 [SMILES: Cc1nc2nc(C3CCOC(c4cc(C5CC5)n[nH]4)C3)nc(-c3ccc(F)cc3F)c2nc1C] |
| I-444 | 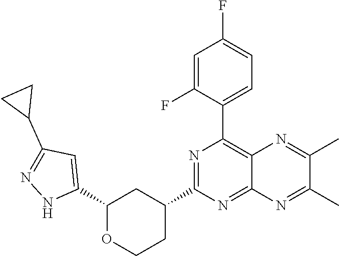 [SMILES: Cc1nc2nc([C@@H]3CCO[C@H](c4cc(C5CC5)n[nH]4)C3)nc(-c3ccc(F)cc3F)c2nc1C] |
| I-445 | 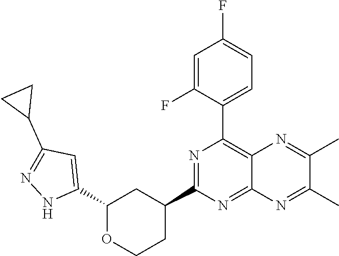 [SMILES: Cc1nc2nc([C@H]3CCO[C@H](c4cc(C5CC5)n[nH]4)C3)nc(-c3ccc(F)cc3F)c2nc1C] |
| I-446 | 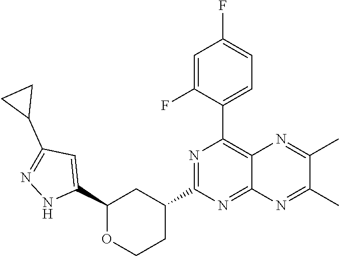 [SMILES: Cc1nc2nc([C@@H]3CCO[C@@H](c4cc(C5CC5)n[nH]4)C3)nc(-c3ccc(F)cc3F)c2nc1C] |
| I-447 | 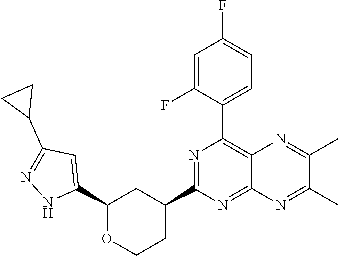 [SMILES: Cc1nc2nc([C@H]3CCO[C@@H](c4cc(C5CC5)n[nH]4)C3)nc(-c3ccc(F)cc3F)c2nc1C] |
| I-448 | 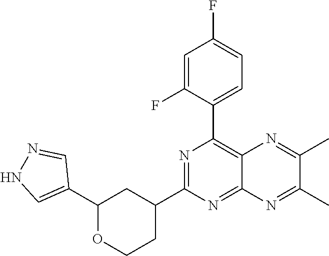 [SMILES: Cc1nc2nc(C3CCOC(c4cn[nH]c4)C3)nc(-c3ccc(F)cc3F)c2nc1C] |
| I-449 | 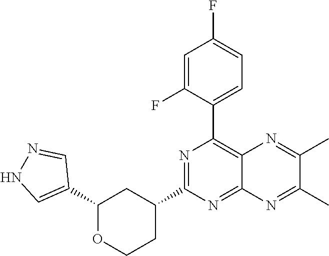 [SMILES: Cc1nc2nc([C@@H]3CCO[C@H](c4cn[nH]c4)C3)nc(-c3ccc(F)cc3F)c2nc1C] |
| I-450 | 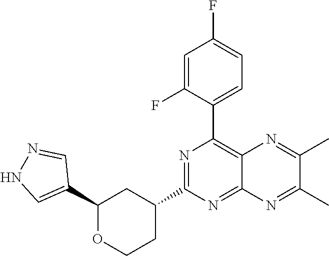 [SMILES: Cc1nc2nc([C@@H]3CCO[C@@H](c4cn[nH]c4)C3)nc(-c3ccc(F)cc3F)c2nc1C] |
| I-451 | 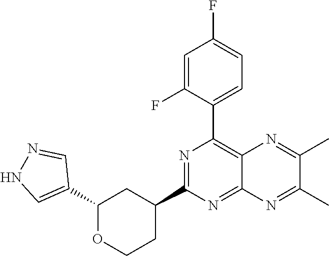 [SMILES: Cc1nc2nc([C@H]3CCO[C@H](c4cn[nH]c4)C3)nc(-c3ccc(F)cc3F)c2nc1C] |
| I-452 | 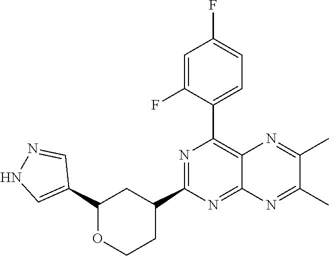 |
| I-453 | 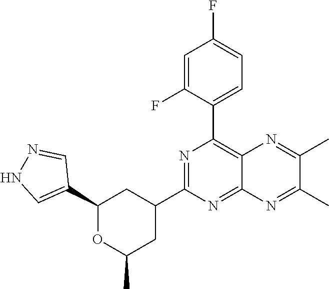 [SMILES: Cc1nc2nc(C3C[C@@H](C)O[C@@H](c4cn[nH]c4)C3)nc(-c3ccc(F)cc3F)c2nc1C] |
| I-454 | 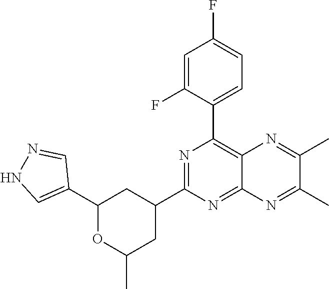 [SMILES: Cc1nc2nc(C3CC(C)OC(c4cn[nH]c4)C3)nc(-c3ccc(F)cc3F)c2nc1C] |
| I-455 | 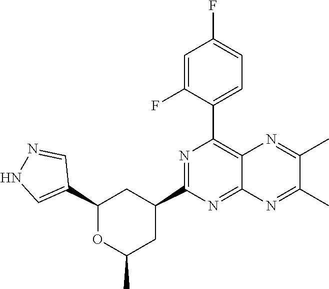 [SMILES: Cc1nc2nc([C@H]3C[C@@H](C)O[C@@H](c4cn[nH]c4)C3)nc(-c3ccc(F)cc3F)c2nc1C] |
| I-456 | 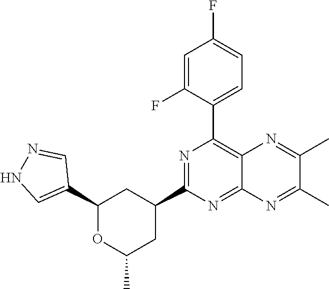 [SMILES: Cc1nc2nc([C@H]3C[C@H](C)O[C@@H](c4cn[nH]c4)C3)nc(-c3ccc(F)cc3F)c2nc1C] |
| I-457 | 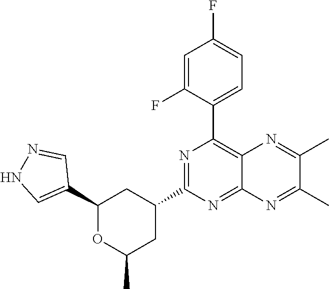 [SMILES: Cc1nc2nc([C@@H]3C[C@@H](C)O[C@@H](c4cn[nH]c4)C3)nc(-c3ccc(F)cc3F)c2nc1C] |
| I-458 | 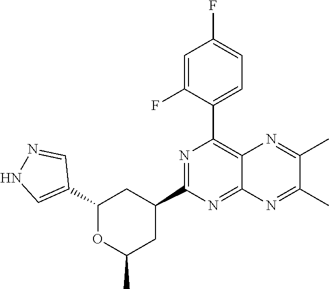 [SMILES: Cc1nc2nc([C@H]3C[C@@H](C)O[C@H](c4cn[nH]c4)C3)nc(-c3ccc(F)cc3F)c2nc1C] |
| I-459 | 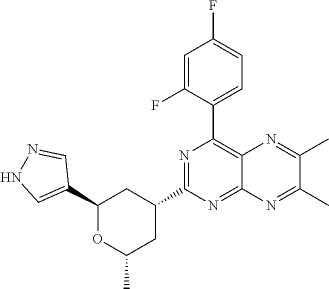 [SMILES: Cc1nc2nc([C@@H]3C[C@H](C)O[C@@H](c4cn[nH]c4)C3)nc(-c3ccc(F)cc3F)c2nc1C] |
| I-460 | 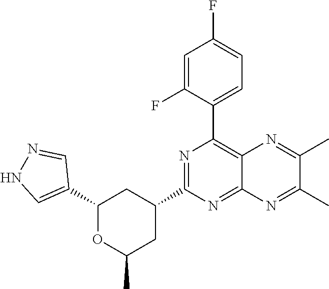 [SMILES: Cc1nc2nc([C@@H]3C[C@@H](C)O[C@H](c4cn[nH]c4)C3)nc(-c3ccc(F)cc3F)c2nc1C] |
| I-461 | 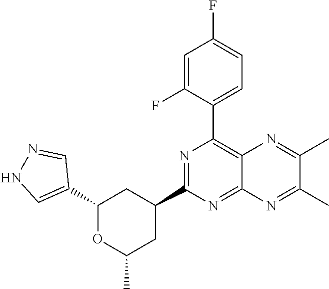 [SMILES: Cc1nc2nc([C@@H]3C[C@@H](c4cn[nH]c4)O[C@@H](C)C3)nc(-c3ccc(F)cc3F)c2nc1C] |
| I-462 | 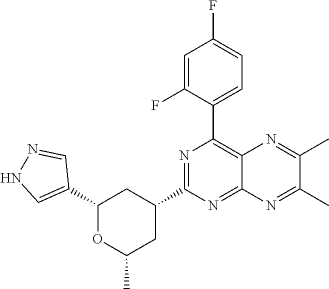 [SMILES: Cc1nc2nc([C@H]3C[C@@H](c4cn[nH]c4)O[C@@H](C)C3)nc(-c3ccc(F)cc3F)c2nc1C] |
| I-463 | 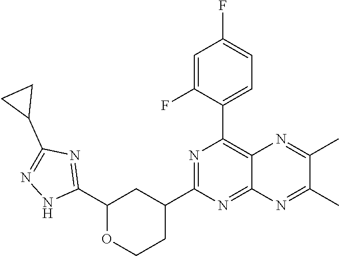 [SMILES: Cc1nc2nc(C3CCOC(c4nc(C5CC5)n[nH]4)C3)nc(-c3ccc(F)cc3F)c2nc1C] |
| I-464 | 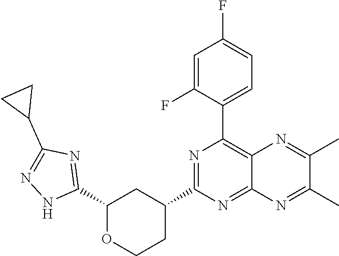 [SMILES: Cc1nc2nc([C@@H]3CCO[C@H](c4nc(C5CC5)n[nH]4)C3)nc(-c3ccc(F)cc3F)c2nc1C] |
| I-465 | 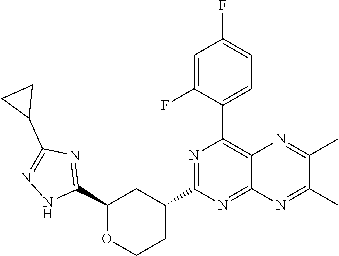 [SMILES: Cc1nc2nc([C@@H]3CCO[C@@H](c4nc(C5CC5)n[nH]4)C3)nc(-c3ccc(F)cc3F)c2nc1C] |
| I-466 | 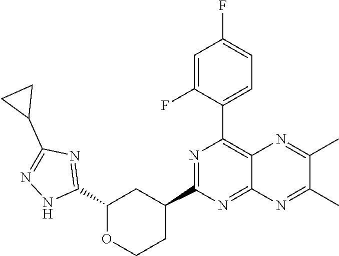 [SMILES: Cc1nc2nc([C@H]3CCO[C@H](c4nc(C5CC5)n[nH]4)C3)nc(-c3ccc(F)cc3F)c2nc1C] |
| I-467 | 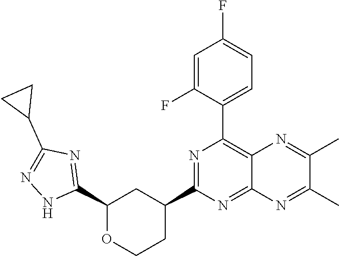 [SMILES: Cc1nc2nc([C@H]3CCO[C@@H](c4nc(C5CC5)n[nH]4)C3)nc(-c3ccc(F)cc3F)c2nc1C] |
| I-468 | 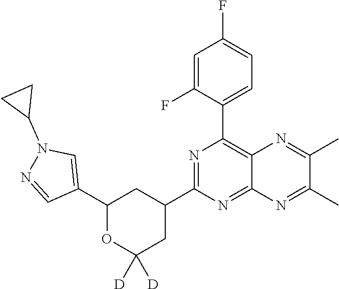 [SMILES: [2H]C1([2H])CC(c2nc(-c3ccc(F)cc3F)c3nc(C)c(C)nc3n2)CC(c2cnn(C3CC3)c2)O1] |
| I-469 | 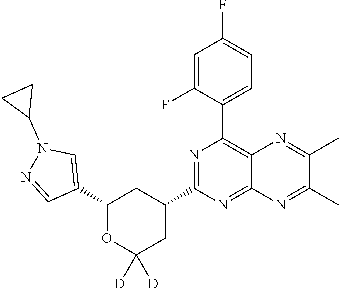 [SMILES: [2H]C1([2H])C[C@@H](c2nc(-c3ccc(F)cc3F)c3nc(C)c(C)nc3n2)C[C@@H](c2cnn(C3CC3)c2)O1] |
| I-470 | 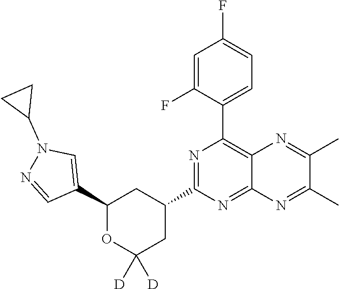 [SMILES: [2H]C1([2H])C[C@@H](c2nc(-c3ccc(F)cc3F)c3nc(C)c(C)nc3n2)C[C@H](c2cnn(C3CC3)c2)O1] |
| I-471 | 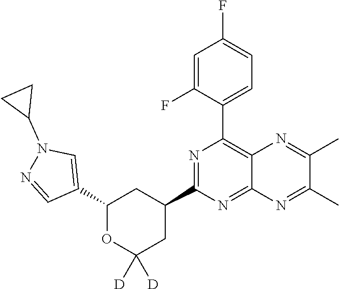 [SMILES: [2H]C1([2H])C[C@H](c2nc(-c3ccc(F)cc3F)c3nc(C)c(C)nc3n2)C[C@@H](c2cnn(C3CC3)c2)O1] |
| I-472 | 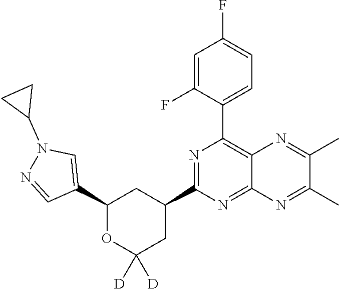 [SMILES: [2H]C1([2H])C[C@H](c2nc(-c3ccc(F)cc3F)c3nc(C)c(C)nc3n2)C[C@H](c2cnn(C3CC3)c2)O1] |
| I-473 | 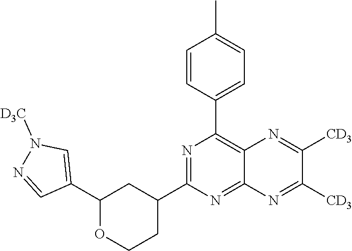 [SMILES: [2H]C([2H])([2H])c1nc2nc(C3CCOC(c4cnn(C([2H])([2H])[2H])c4)C3)nc(-c3ccc(C)cc3)c2nc1C([2H])([2H])[2H]] |
| I-474 | 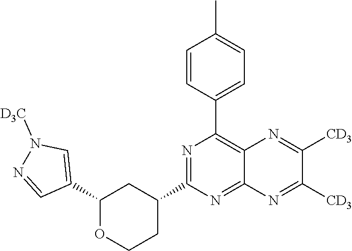 [SMILES: [2H]C([2H])([2H])c1nc2nc([C@@H]3CCO[C@H](c4cnn(C([2H])([2H])[2H])c4)C3)nc(-c3ccc(C)cc3)c2nc1C([2H])([2H])[2H]] |
| I-475 | 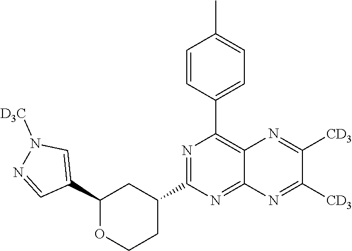 [SMILES: [2H]C([2H])([2H])c1nc2nc([C@@H]3CCO[C@@H](c4cnn(C([2H])([2H])[2H])c4)C3)nc(-c3ccc(C)cc3)c2nc1C([2H])([2H])[2H]] |
| I-476 | 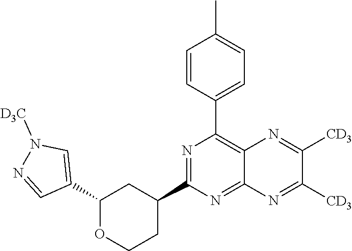 [SMILES: [2H]C([2H])([2H])c1nc2nc([C@H]3CCO[C@H](c4cnn(C([2H])([2H])[2H])c4)C3)nc(-c3ccc(C)cc3)c2nc1C([2H])([2H])[2H]] |
| I-477 | 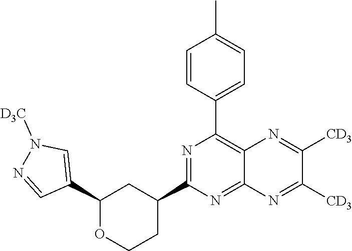 [SMILES: [2H]C([2H])([2H])c1nc2nc([C@H]3CCO[C@@H](c4cnn(C([2H])([2H])[2H])c4)C3)nc(-c3ccc(C)cc3)c2nc1C([2H])([2H])[2H]] |
| I-478 | 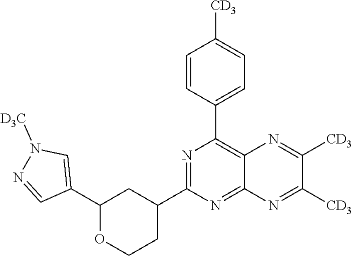 [SMILES: [2H]C([2H])([2H])c1ccc(-c2nc(C3CCOC(c4cnn(C([2H])([2H])[2H])c4)C3)nc3nc(C([2H])([2H])[2H])c(C([2H])([2H])[2H])nc23)cc1] |
| I-479 | 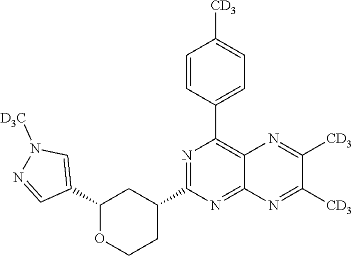 [SMILES: [2H]C([2H])([2H])c1ccc(-c2nc([C@@H]3CCO[C@H](c4cnn(C([2H])([2H])[2H])c4)C3)nc3nc(C([2H])([2H])[2H])c(C([2H])([2H])[2H])nc23)cc1] |
| I-480 | 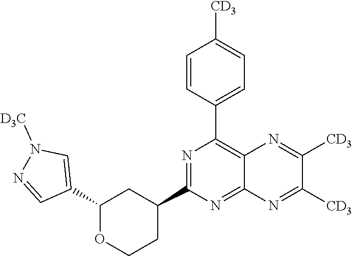 [SMILES: [2H]C([2H])([2H])c1ccc(-c2nc([C@H]3CCO[C@H](c4cnn(C([2H])([2H])[2H])c4)C3)nc3nc(C([2H])([2H])[2H])c(C([2H])([2H])[2H])nc23)cc1] |
| I-481 | 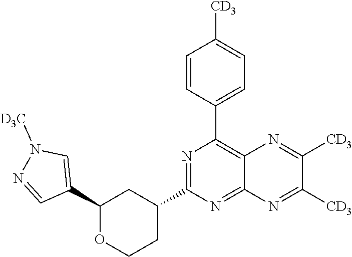 [SMILES: [2H]C([2H])([2H])c1ccc(-c2nc([C@@H]3CCO[C@@H](c4cnn(C([2H])([2H])[2H])c4)C3)nc3nc(C([2H])([2H])[2H])c(C([2H])([2H])[2H])nc23)cc1] |
| I-482 | 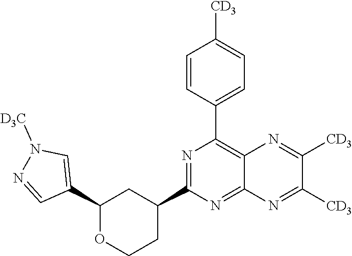 [SMILES: [2H]C([2H])([2H])c1ccc(-c2nc([C@H]3CCO[C@@H](c4cnn(C([2H])([2H])[2H])c4)C3)nc3nc(C([2H])([2H])[2H])c(C([2H])([2H])[2H])nc23)cc1] |
| I-483 | 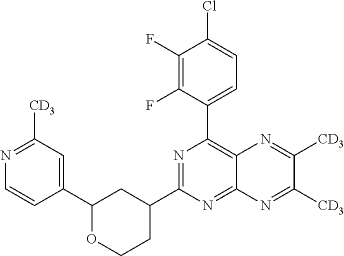 [SMILES: [2H]C([2H])([2H])c1cc(C2CC(c3nc(-c4ccc(Cl)c(F)c4F)c4nc(C([2H])([2H])[2H])c(C([2H])([2H])[2H])nc4n3)CCO2)ccn1] |
| I-484 | 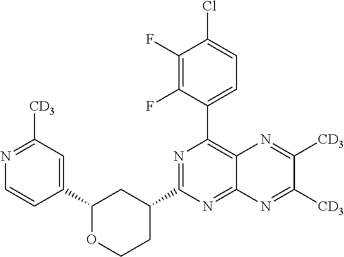 [SMILES: [2H]C([2H])([2H])c1cc([C@@H]2C[C@H](c3nc(-c4ccc(Cl)c(F)c4F)c4nc(C([2H])([2H])[2H])c(C([2H])([2H])[2H])nc4n3)CCO2)ccn1] |
| I-485 | 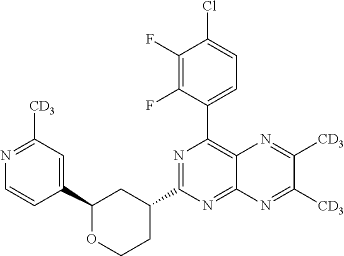 [SMILES: [2H]C([2H])([2H])c1cc([C@H]2C[C@H](c3nc(-c4ccc(Cl)c(F)c4F)c4nc(C([2H])([2H])[2H])c(C([2H])([2H])[2H])nc4n3)CCO2)ccn1] |
| I-486 | 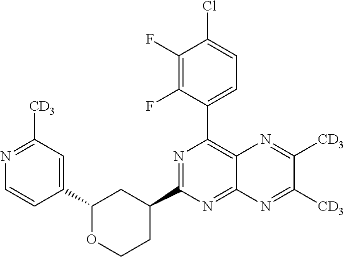 [SMILES: [2H]C([2H])([2H])c1cc([C@@H]2C[C@@H](c3nc(-c4ccc(Cl)c(F)c4F)c4nc(C([2H])([2H])[2H])c(C([2H])([2H])[2H])nc4n3)CCO2)ccn1] |
| I-487 | 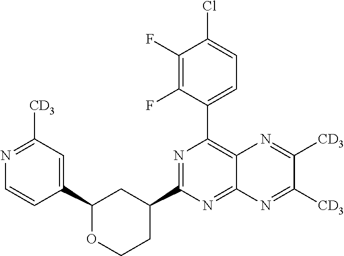 [SMILES: [2H]C([2H])([2H])c1cc([C@H]2C[C@@H](c3nc(-c4ccc(Cl)c(F)c4F)c4nc(C([2H])([2H])[2H])c(C([2H])([2H])[2H])nc4n3)CCO2)ccn1] |
| I-488 | 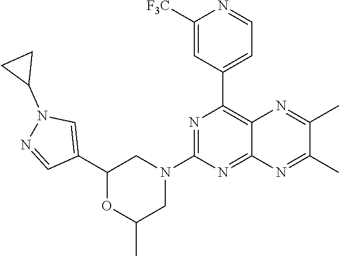 [SMILES: Cc1nc2nc(N3CC(C)OC(c4cnn(C5CC5)c4)C3)nc(-c3ccnc(C(F)(F)F)c3)c2nc1C] |
| I-489 | 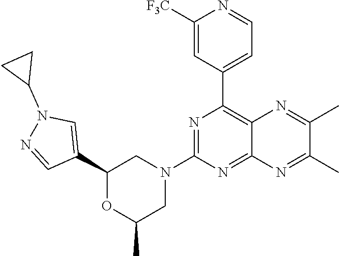 [SMILES: Cc1nc2nc(N3C[C@@H](C)O[C@@H](c4cnn(C5CC5)c4)C3)nc(-c3ccnc(C(F)(F)F)c3)c2nc1C] |
| I-490 | 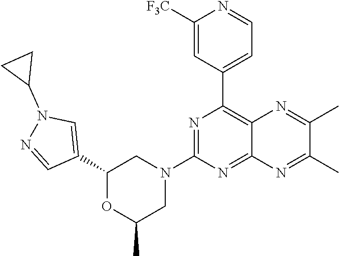 [SMILES: Cc1nc2nc(N3C[C@@H](C)O[C@H](c4cnn(C5CC5)c4)C3)nc(-c3ccnc(C(F)(F)F)c3)c2nc1C] |
| I-491 | 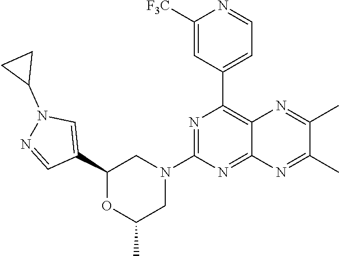 [SMILES: Cc1nc2nc(N3C[C@H](C)O[C@@H](c4cnn(C5CC5)c4)C3)nc(-c3ccnc(C(F)(F)F)c3)c2nc1C] |
| I-492 | 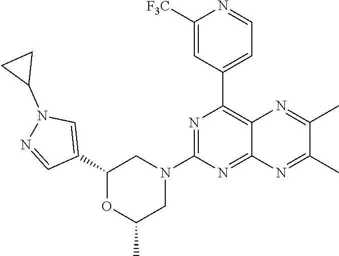 [SMILES: Cc1nc2nc(N3C[C@@H](c4cnn(C5CC5)c4)O[C@@H](C)C3)nc(-c3ccnc(C(F)(F)F)c3)c2nc1C] |
| I-493 | 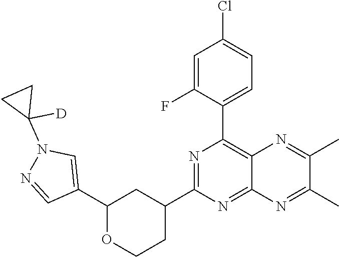 [SMILES: [2H]C1(n2cc(C3CC(c4nc(-c5ccc(Cl)cc5F)c5nc(C)c(C)nc5n4)CCO3)cn2)CC1] |
| I-494 | 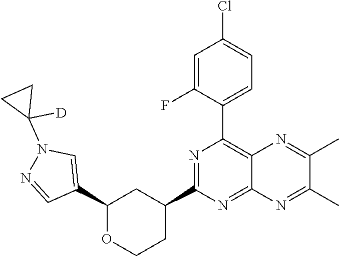 [SMILES: [2H]C1(n2cc([C@H]3C[C@@H](c4nc(-c5ccc(Cl)cc5F)c5nc(C)c(C)nc5n4)CCO3)cn2)CC1] |
| I-495 | 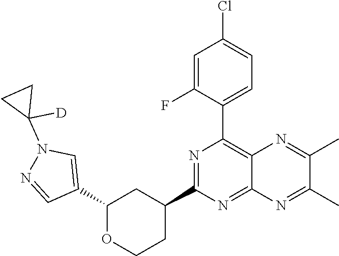 [SMILES: [2H]C1(n2cc([C@@H]3C[C@@H](c4nc(-c5ccc(Cl)cc5F)c5nc(C)c(C)nc5n4)CCO3)cn2)CC1] |
| I-496 | 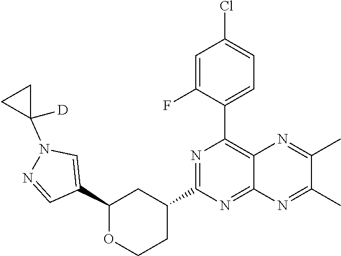 [SMILES: [2H]C1(n2cc([C@H]3C[C@H](c4nc(-c5ccc(Cl)cc5F)c5nc(C)c(C)nc5n4)CCO3)cn2)CC1] |
| I-497 | 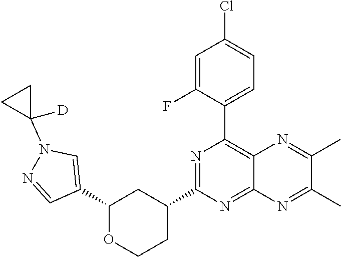 [SMILES: [2H]C1(n2cc([C@@H]3C[C@H](c4nc(-c5ccc(Cl)cc5F)c5nc(C)c(C)nc5n4)CCO3)cn2)CC1] |
| I-498 | 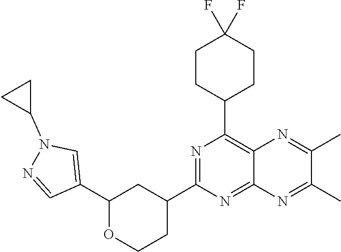 [SMILES: Cc1nc2nc(C3CCOC(c4cnn(C5CC5)c4)C3)nc(C3CCC(F)(F)CC3)c2nc1C] |
| I-499 | 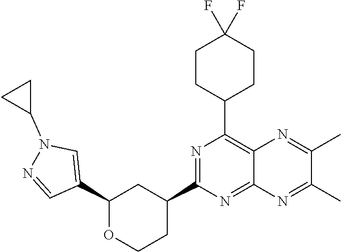 [SMILES: Cc1nc2nc([C@H]3CCO[C@@H](c4cnn(C5CC5)c4)C3)nc(C3CCC(F)(F)CC3)c2nc1C] |
| I-500 | 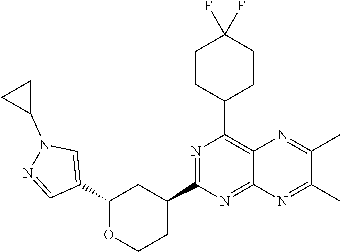 [SMILES: Cc1nc2nc([C@H]3CCO[C@H](c4cnn(C5CC5)c4)C3)nc(C3CCC(F)(F)CC3)c2nc1C] |
| I-501 | 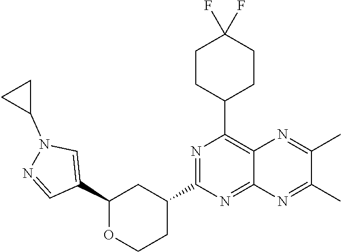 [SMILES: Cc1nc2nc([C@@H]3CCO[C@@H](c4cnn(C5CC5)c4)C3)nc(C3CCC(F)(F)CC3)c2nc1C] |
| I-502 | 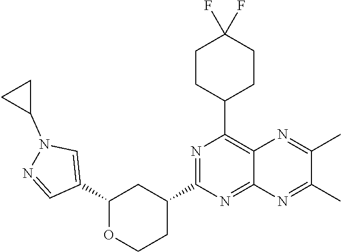 [SMILES: Cc1nc2nc([C@@H]3CCO[C@H](c4cnn(C5CC5)c4)C3)nc(C3CCC(F)(F)CC3)c2nc1C] |
| I-503 | 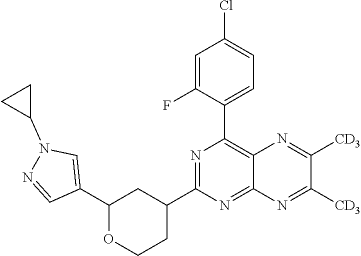 [SMILES: [2H]C([2H])([2H])c1nc2nc(C3CCOC(c4cnn(C5CC5)c4)C3)nc(-c3ccc(Cl)cc3F)c2nc1C([2H])([2H])[2H]] |
| I-504 | 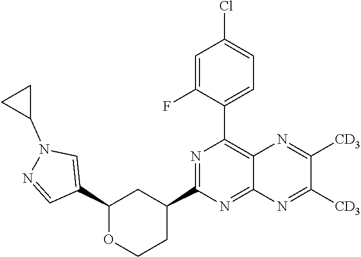 [SMILES: [2H]C([2H])([2H])c1nc2nc([C@H]3CCO[C@@H](c4cnn(C5CC5)c4)C3)nc(-c3ccc(Cl)cc3F)c2nc1C([2H])([2H])[2H]] |
| I-505 | 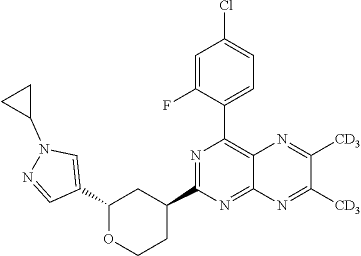 [SMILES: [2H]C([2H])([2H])c1nc2nc([C@H]3CCO[C@H](c4cnn(C5CC5)c4)C3)nc(-c3ccc(Cl)cc3F)c2nc1C([2H])([2H])[2H]] |
| I-506 | 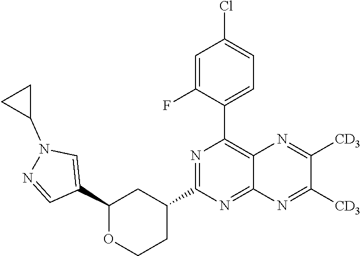 [SMILES: [2H]C([2H])([2H])c1nc2nc([C@@H]3CCO[C@@H](c4cnn(C5CC5)c4)C3)nc(-c3ccc(Cl)cc3F)c2nc1C([2H])([2H])[2H]] |
| I-507 | 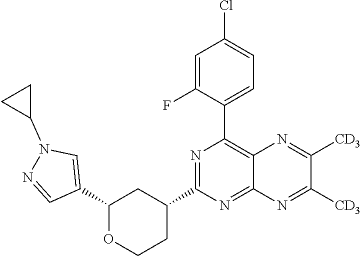 [SMILES: [2H]C([2H])([2H])c1nc2nc([C@@H]3CCO[C@H](c4cnn(C5CC5)c4)C3)nc(-c3ccc(Cl)cc3F)c2nc1C([2H])([2H])[2H]] |
| I-508 | 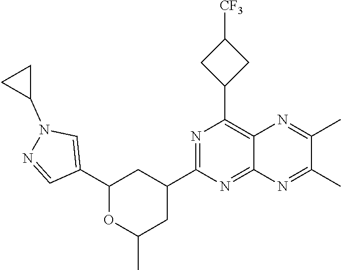 [SMILES: Cc1nc2nc(C3CC(C)OC(c4cnn(C5CC5)c4)C3)nc(C3CC(C(F)(F)F)C3)c2nc1C] |
| I-509 | 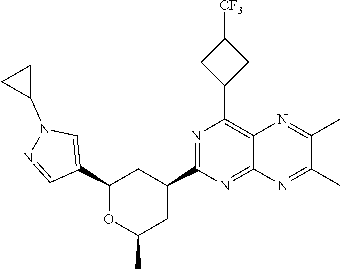 [SMILES: Cc1nc2nc([C@H]3C[C@@H](C)O[C@@H](c4cnn(C5CC5)c4)C3)nc(C3CC(C(F)(F)F)C3)c2nc1C] |
| I-510 | 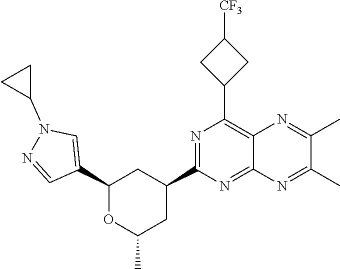 [SMILES: Cc1nc2nc([C@H]3C[C@H](C)O[C@@H](c4cnn(C5CC5)c4)C3)nc(C3CC(C(F)(F)F)C3)c2nc1C] |
| I-511 | 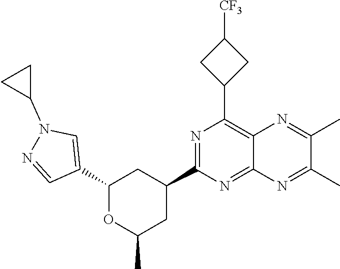 [SMILES: Cc1nc2nc([C@H]3C[C@@H](C)O[C@H](c4cnn(C5CC5)c4)C3)nc(C3CC(C(F)(F)F)C3)c2nc1C] |
| I-512 | 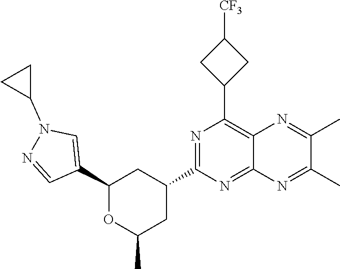 [SMILES: Cc1nc2nc([C@@H]3C[C@@H](C)O[C@@H](c4cnn(C5CC5)c4)C3)nc(C3CC(C(F)(F)F)C3)c2nc1C] |
| I-513 | 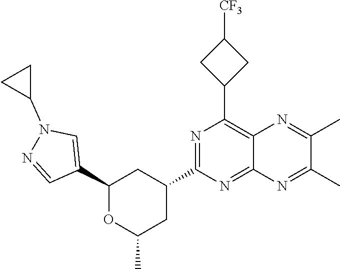 [SMILES: Cc1nc2nc([C@@H]3C[C@H](C)O[C@@H](c4cnn(C5CC5)c4)C3)nc(C3CC(C(F)(F)F)C3)c2nc1C] |
| I-514 | 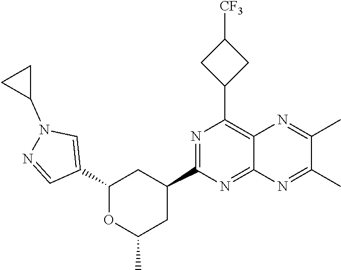 [SMILES: Cc1nc2nc([C@@H]3C[C@@H](c4cnn(C5CC5)c4)O[C@@H](C)C3)nc(C3CC(C(F)(F)F)C3)c2nc1C] |
| I-515 | 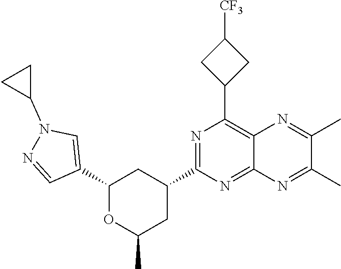 [SMILES: Cc1nc2nc([C@@H]3C[C@@H](C)O[C@H](c4cnn(C5CC5)c4)C3)nc(C3CC(C(F)(F)F)C3)c2nc1C] |
| I-516 | 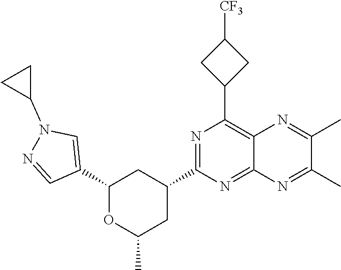 [SMILES: Cc1nc2nc([C@H]3C[C@@H](c4cnn(C5CC5)c4)O[C@@H](C)C3)nc(C3CC(C(F)(F)F)C3)c2nc1C] |
| I-517 | 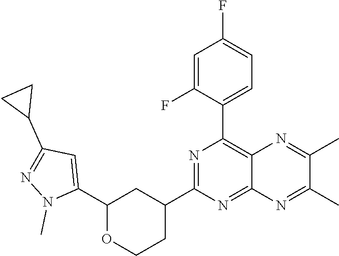 [SMILES: Cc1nc2nc(C3CCOC(c4cc(C5CC5)nn4C)C3)nc(-c3ccc(F)cc3F)c2nc1C] |
| I-518 | 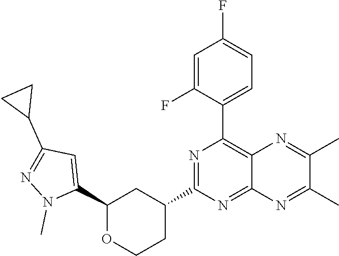 [SMILES: Cc1nc2nc([C@@H]3CCO[C@@H](c4cc(C5CC5)nn4C)C3)nc(-c3ccc(F)cc3F)c2nc1C] |
| I-519 | 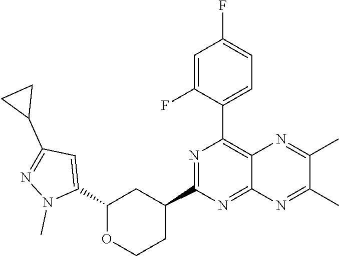 [SMILES: Cc1nc2nc([C@H]3CCO[C@H](c4cc(C5CC5)nn4C)C3)nc(-c3ccc(F)cc3F)c2nc1C] |
| I-520 | 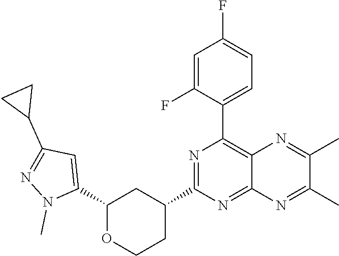 [SMILES: Cc1nc2nc([C@@H]3CCO[C@H](c4cc(C5CC5)nn4C)C3)nc(-c3ccc(F)cc3F)c2nc1C] |
| I-521 | 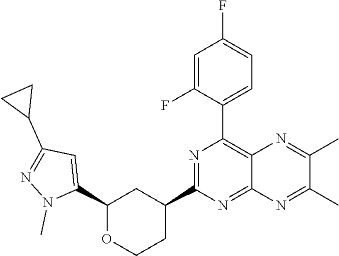 [SMILES: Cc1nc2nc([C@H]3CCO[C@@H](c4cc(C5CC5)nn4C)C3)nc(-c3ccc(F)cc3F)c2nc1C] |
| I-522 | 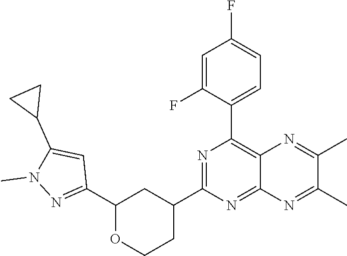 |
| I-523 | 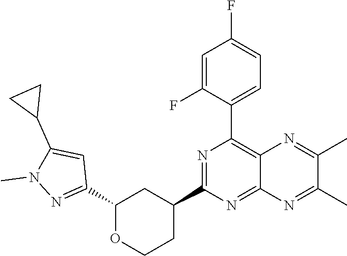 |
| I-524 | 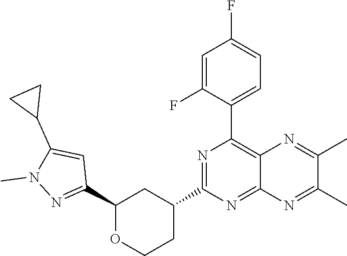 |
| I-525 | 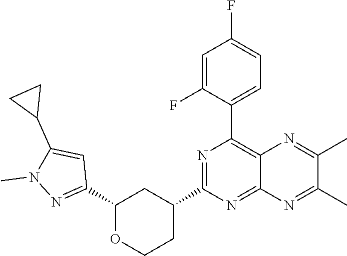 |
| I-526 | 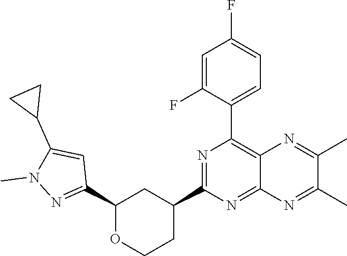 |
| I-527 | 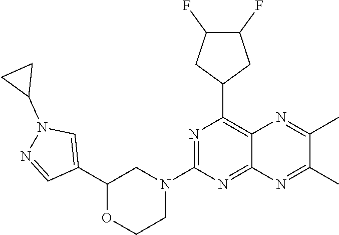 [SMILES: Cc1nc2nc(N3CCOC(c4cnn(C5CC5)c4)C3)nc(C3CC(F)C(F)C3)c2nc1C] |
| I-528 | 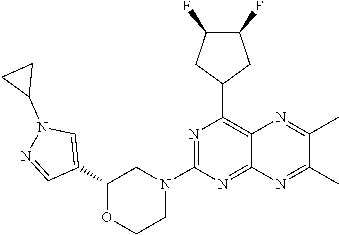 [SMILES: Cc1nc2nc(N3CCO[C@H](c4cnn(C5CC5)c4)C3)nc(C3C[C@@H](F)[C@@H](F)C3)c2nc1C] |
| I-529 | 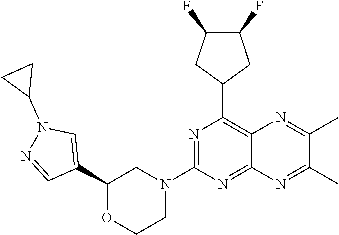 [SMILES: Cc1nc2nc(N3CCO[C@@H](c4cnn(C5CC5)c4)C3)nc(C3C[C@@H](F)[C@@H](F)C3)c2nc1C] |
| I-530 | 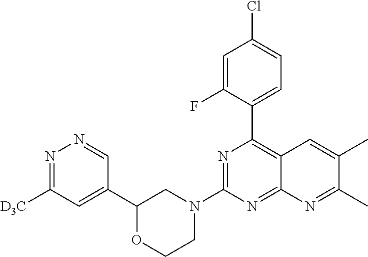 [SMILES: [2H]C([2H])([2H])c1cc(C2CN(c3nc(-c4ccc(Cl)cc4F)c4cc(C)c(C)nc4n3)CCO2)cnn1] |
| I-531 | 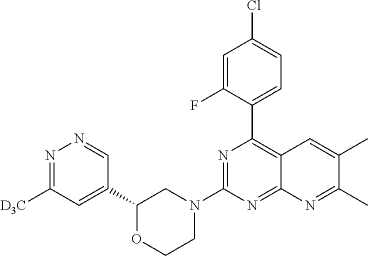 [SMILES: [2H]C([2H])([2H])c1cc([C@@H]2CN(c3nc(-c4ccc(Cl)cc4F)c4cc(C)c(C)nc4n3)CCO2)cnn1] |
| I-532 | 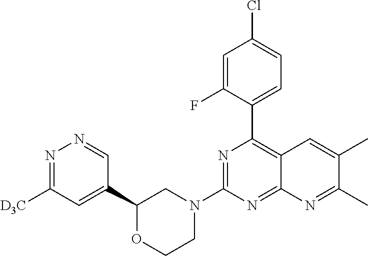 [SMILES: [2H]C([2H])([2H])c1cc([C@H]2CN(c3nc(-c4ccc(Cl)cc4F)c4cc(C)c(C)nc4n3)CCO2)cnn1] |
| I-533 | 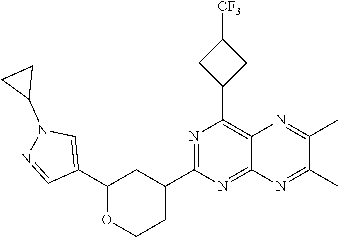 [SMILES: Cc1nc2nc(C3CCOC(c4cnn(C5CC5)c4)C3)nc(C3CC(C(F)(F)F)C3)c2nc1C] |
| I-534 | 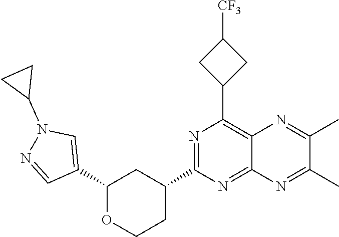 [SMILES: Cc1nc2nc([C@@H]3CCO[C@H](c4cnn(C5CC5)c4)C3)nc(C3CC(C(F)(F)F)C3)c2nc1C] |
| I-535 | 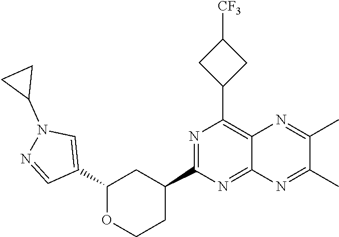 [SMILES: Cc1nc2nc([C@H]3CCO[C@H](c4cnn(C5CC5)c4)C3)nc(C3CC(C(F)(F)F)C3)c2nc1C] |
| I-536 | 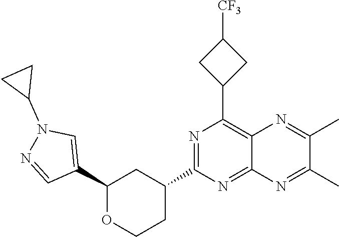 [SMILES: Cc1nc2nc([C@@H]3CCO[C@@H](c4cnn(C5CC5)c4)C3)nc(C3CC(C(F)(F)F)C3)c2nc1C] |
| I-537 | 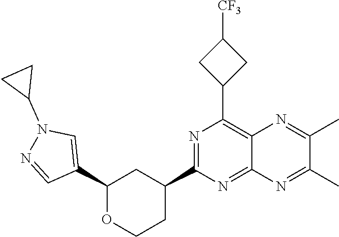 [SMILES: Cc1nc2nc([C@H]3CCO[C@@H](c4cnn(C5CC5)c4)C3)nc(C3CC(C(F)(F)F)C3)c2nc1C] |
| I-538 | 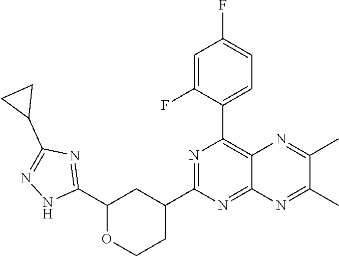 [SMILES: Cc1nc2nc(C3CCOC(c4nc(C5CC5)n[nH]4)C3)nc(-c3ccc(F)cc3F)c2nc1C] |
| I-539 | 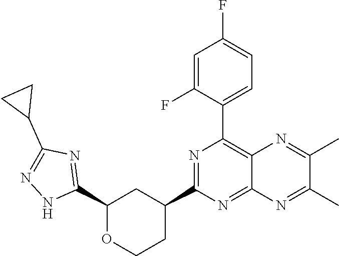 [SMILES: Cc1nc2nc([C@H]3CCO[C@@H](c4nc(C5CC5)n[nH]4)C3)nc(-c3ccc(F)cc3F)c2nc1C] |
| I-540 | 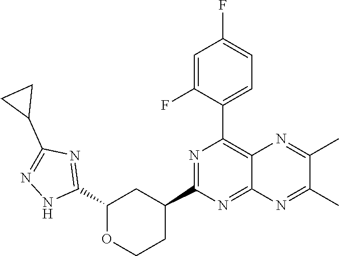 [SMILES: Cc1nc2nc([C@H]3CCO[C@H](c4nc(C5CC5)n[nH]4)C3)nc(-c3ccc(F)cc3F)c2nc1C] |
| I-541 | 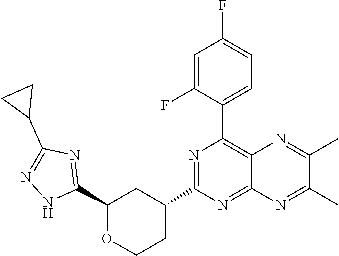 [SMILES: Cc1nc2nc([C@@H]3CCO[C@@H](c4nc(C5CC5)n[nH]4)C3)nc(-c3ccc(F)cc3F)c2nc1C] |
| I-542 | 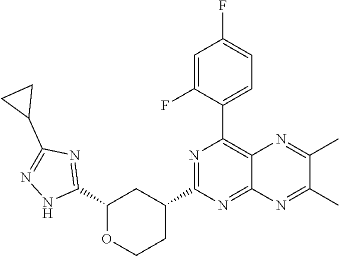 [SMILES: Cc1nc2nc([C@@H]3CCO[C@H](c4nc(C5CC5)n[nH]4)C3)nc(-c3ccc(F)cc3F)c2nc1C] |
| I-543 | 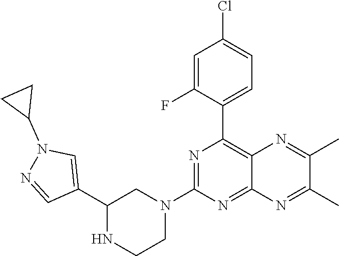 [SMILES: Cc1nc2nc(N3CCNC(c4cnn(C5CC5)c4)C3)nc(-c3ccc(Cl)cc3F)c2nc1C] |
| I-544 | 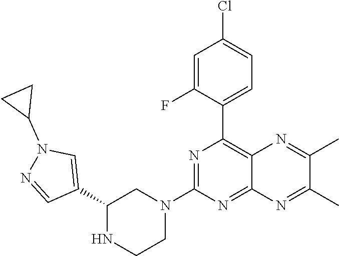 [SMILES: Cc1nc2nc(N3CCN[C@H](c4cnn(C5CC5)c4)C3)nc(-c3ccc(Cl)cc3F)c2nc1C] |
| I-545 | 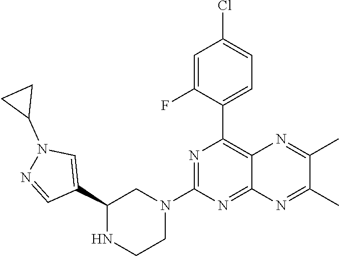 [SMILES: Cc1nc2nc(N3CCN[C@@H](c4cnn(C5CC5)c4)C3)nc(-c3ccc(Cl)cc3F)c2nc1C] |
| I-546 | 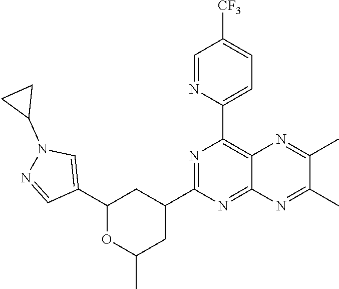 [SMILES: Cc1nc2nc(C3CC(C)OC(c4cnn(C5CC5)c4)C3)nc(-c3ccc(C(F)(F)F)cn3)c2nc1C] |
| I-547 | 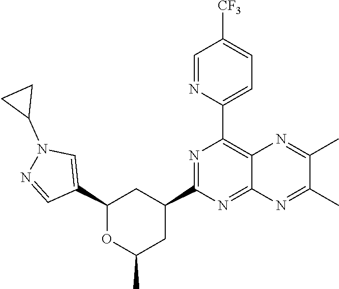 [SMILES: Cc1nc2nc([C@H]3C[C@@H](C)O[C@@H](c4cnn(C5CC5)c4)C3)nc(-c3ccc(C(F)(F)F)cn3)c2nc1C] |
| I-548 | 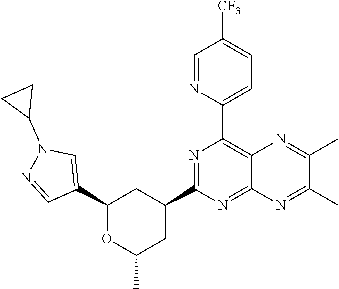 [SMILES: Cc1nc2nc([C@H]3C[C@H](C)O[C@@H](c4cnn(C5CC5)c4)C3)nc(-c3ccc(C(F)(F)F)cn3)c2nc1C] |
| I-549 | 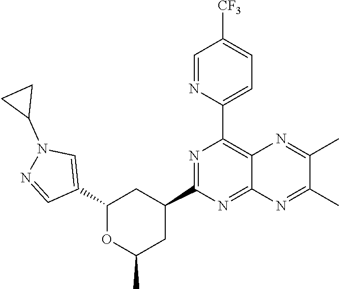 [SMILES: Cc1nc2nc([C@H]3C[C@@H](C)O[C@H](c4cnn(C5CC5)c4)C3)nc(-c3ccc(C(F)(F)F)cn3)c2nc1C] |
| I-550 | 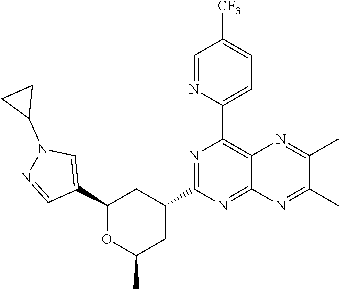 [SMILES: Cc1nc2nc([C@@H]3C[C@@H](C)O[C@@H](c4cnn(C5CC5)c4)C3)nc(-c3ccc(C(F)(F)F)cn3)c2nc1C] |
| I-551 | 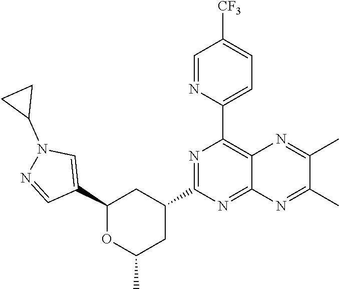 [SMILES: Cc1nc2nc([C@@H]3C[C@H](C)O[C@@H](c4cnn(C5CC5)c4)C3)nc(-c3ccc(C(F)(F)F)cn3)c2nc1C] |
| I-552 | 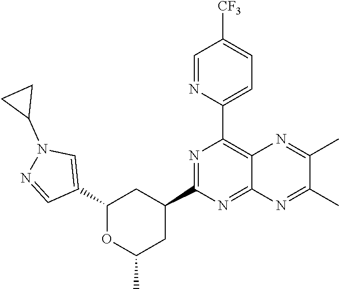 [SMILES: Cc1nc2nc([C@@H]3C[C@@H](c4cnn(C5CC5)c4)O[C@@H](C)C3)nc(-c3ccc(C(F)(F)F)cn3)c2nc1C] |
| I-553 | 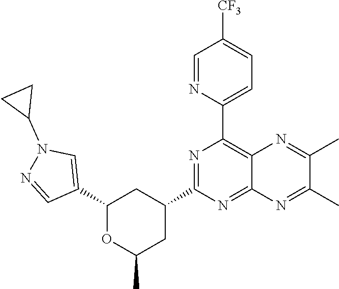 [SMILES: Cc1nc2nc([C@@H]3C[C@@H](C)O[C@H](c4cnn(C5CC5)c4)C3)nc(-c3ccc(C(F)(F)F)cn3)c2nc1C] |
| I-554 | 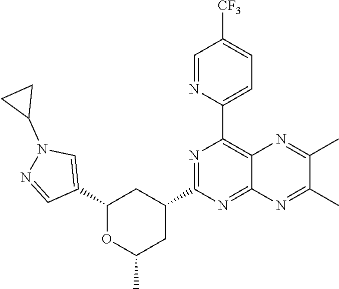 [SMILES: Cc1nc2nc([C@H]3C[C@@H](c4cnn(C5CC5)c4)O[C@@H](C)C3)nc(-c3ccc(C(F)(F)F)cn3)c2nc1C] |
| I-555 | 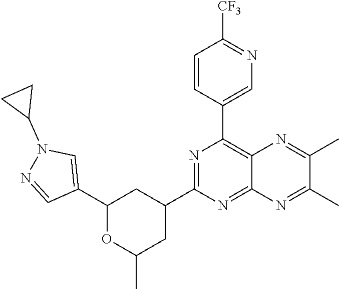 [SMILES: Cc1nc2nc(C3CC(C)OC(c4cnn(C5CC5)c4)C3)nc(-c3ccc(C(F)(F)F)nc3)c2nc1C] |
| I-556 | 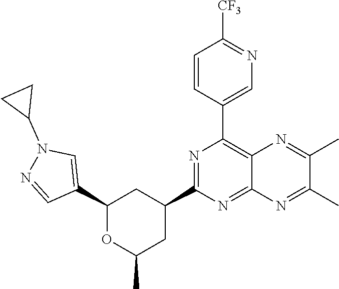 [SMILES: Cc1nc2nc([C@H]3C[C@@H](C)O[C@@H](c4cnn(C5CC5)c4)C3)nc(-c3ccc(C(F)(F)F)nc3)c2nc1C] |
| I-557 | 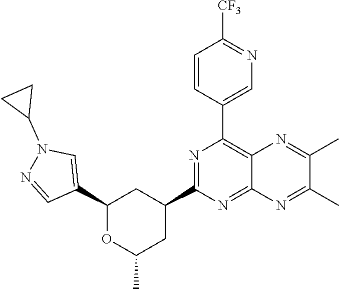 [SMILES: Cc1nc2nc([C@H]3C[C@H](C)O[C@@H](c4cnn(C5CC5)c4)C3)nc(-c3ccc(C(F)(F)F)nc3)c2nc1C] |
| I-558 | 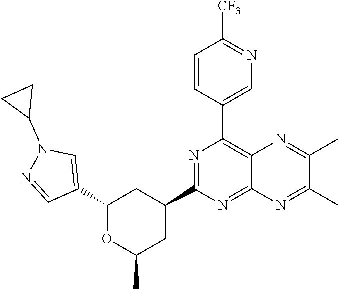 [SMILES: Cc1nc2nc([C@H]3C[C@@H](C)O[C@H](c4cnn(C5CC5)c4)C3)nc(-c3ccc(C(F)(F)F)nc3)c2nc1C] |
| I-559 | 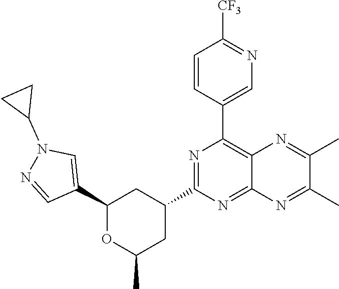 [SMILES: Cc1nc2nc([C@@H]3C[C@@H](C)O[C@@H](c4cnn(C5CC5)c4)C3)nc(-c3ccc(C(F)(F)F)nc3)c2nc1C] |
| I-560 | 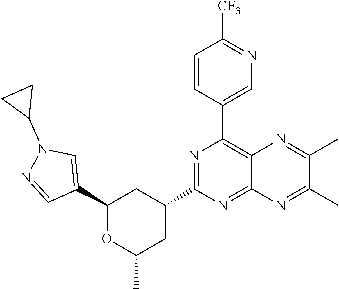 [SMILES: Cc1nc2nc([C@@H]3C[C@H](C)O[C@@H](c4cnn(C5CC5)c4)C3)nc(-c3ccc(C(F)(F)F)nc3)c2nc1C] |
| I-561 | 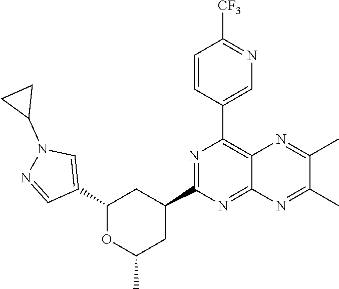 [SMILES: Cc1nc2nc([C@@H]3C[C@@H](c4cnn(C5CC5)c4)O[C@@H](C)C3)nc(-c3ccc(C(F)(F)F)nc3)c2nc1C] |
| I-562 | 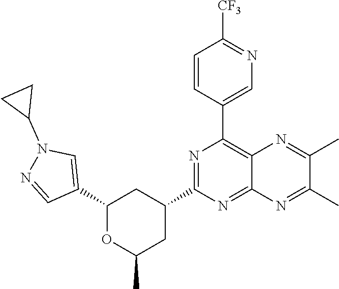 [SMILES: Cc1nc2nc([C@@H]3C[C@@H](C)O[C@H](c4cnn(C5CC5)c4)C3)nc(-c3ccc(C(F)(F)F)nc3)c2nc1C] |
| I-563 | 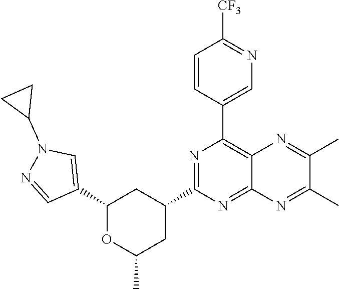 [SMILES: Cc1nc2nc([C@H]3C[C@@H](c4cnn(C5CC5)c4)O[C@@H](C)C3)nc(-c3ccc(C(F)(F)F)nc3)c2nc1C] |
| I-564 | 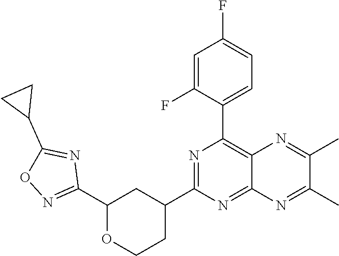 [SMILES: Cc1nc2nc(C3CCOC(c4noc(C5CC5)n4)C3)nc(-c3ccc(F)cc3F)c2nc1C] |
| I-565 | 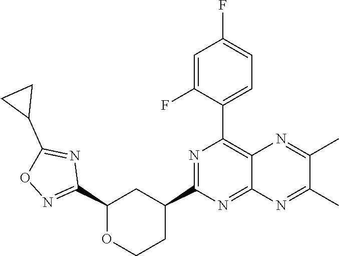 [SMILES: Cc1nc2nc([C@H]3CCO[C@@H](c4noc(C5CC5)n4)C3)nc(-c3ccc(F)cc3F)c2nc1C] |
| I-566 | 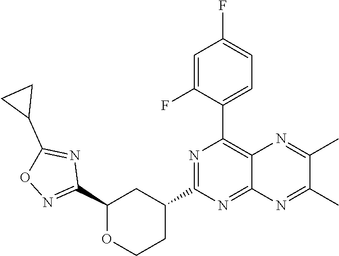 [SMILES: Cc1nc2nc([C@@H]3CCO[C@@H](c4noc(C5CC5)n4)C3)nc(-c3ccc(F)cc3F)c2nc1C] |
| I-567 | 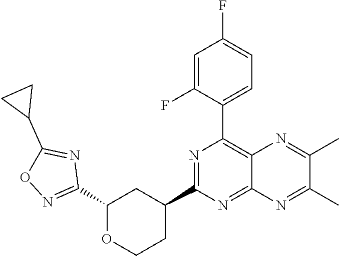 [SMILES: Cc1nc2nc([C@H]3CCO[C@H](c4noc(C5CC5)n4)C3)nc(-c3ccc(F)cc3F)c2nc1C] |
| I-568 | 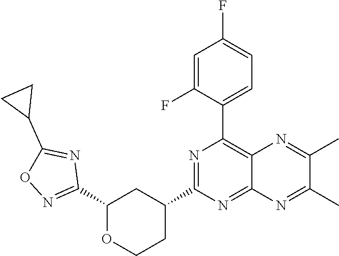 [SMILES: Cc1nc2nc([C@@H]3CCO[C@H](c4noc(C5CC5)n4)C3)nc(-c3ccc(F)cc3F)c2nc1C] |
| I-569 | 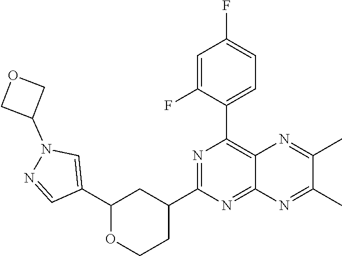 [SMILES: Cc1nc2nc(C3CCOC(c4cnn(C5COC5)c4)C3)nc(-c3ccc(F)cc3F)c2nc1C] |
| I-570 | 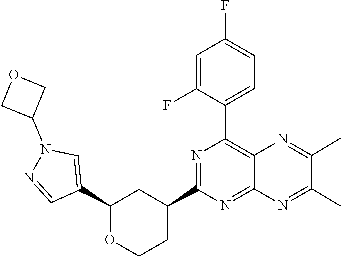 [SMILES: Cc1nc2nc([C@H]3CCO[C@@H](c4cnn(C5COC5)c4)C3)nc(-c3ccc(F)cc3F)c2nc1C] |
| I-571 | 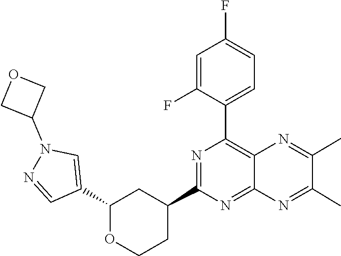 [SMILES: Cc1nc2nc([C@H]3CCO[C@H](c4cnn(C5COC5)c4)C3)nc(-c3ccc(F)cc3F)c2nc1C] |
| I-572 | 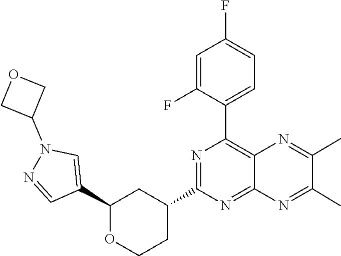 [SMILES: Cc1nc2nc([C@@H]3CCO[C@@H](c4cnn(C5COC5)c4)C3)nc(-c3ccc(F)cc3F)c2nc1C] |
| I-573 | 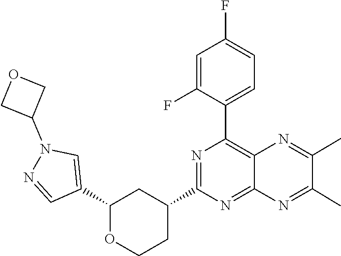 [SMILES: Cc1nc2nc([C@@H]3CCO[C@H](c4cnn(C5COC5)c4)C3)nc(-c3ccc(F)cc3F)c2nc1C] |
| I-574 | 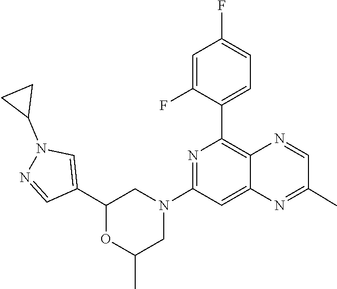 [SMILES: Cc1cnc2c(-c3ccc(F)cc3F)nc(N3CC(C)OC(c4cnn(C5CC5)c4)C3)cc2n1] |
| I-575 | 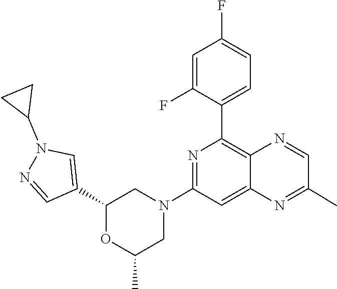 [SMILES: Cc1cnc2c(-c3ccc(F)cc3F)nc(N3C[C@@H](c4cnn(C5CC5)c4)O[C@@H](C)C3)cc2n1] |
| I-576 | 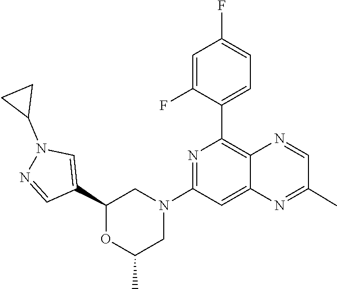 [SMILES: Cc1cnc2c(-c3ccc(F)cc3F)nc(N3C[C@H](C)O[C@@H](c4cnn(C5CC5)c4)C3)cc2n1] |
| I-577 | 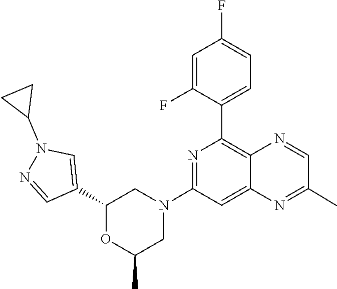 [SMILES: Cc1cnc2c(-c3ccc(F)cc3F)nc(N3C[C@@H](C)O[C@H](c4cnn(C5CC5)c4)C3)cc2n1] |
| I-578 | 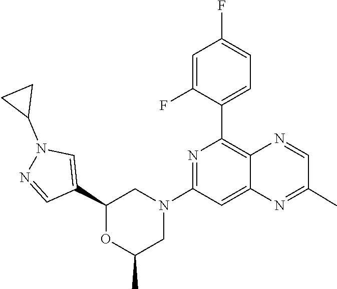 [SMILES: Cc1cnc2c(-c3ccc(F)cc3F)nc(N3C[C@@H](C)O[C@@H](c4cnn(C5CC5)c4)C3)cc2n1] |
| I-579 | 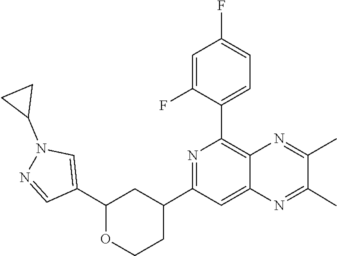 [SMILES: Cc1nc2cc(C3CCOC(c4cnn(C5CC5)c4)C3)nc(-c3ccc(F)cc3F)c2nc1C] |
| I-580 | 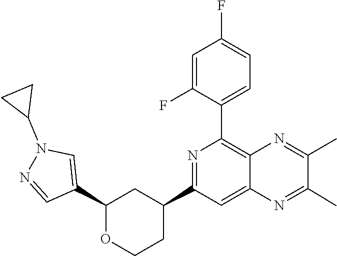 [SMILES: Cc1nc2cc([C@H]3CCO[C@@H](c4cnn(C5CC5)c4)C3)nc(-c3ccc(F)cc3F)c2nc1C] |
| I-581 | 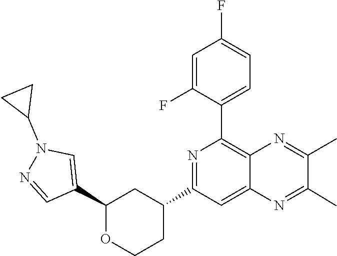 [SMILES: Cc1nc2cc([C@@H]3CCO[C@@H](c4cnn(C5CC5)c4)C3)nc(-c3ccc(F)cc3F)c2nc1C] |
| I-582 | 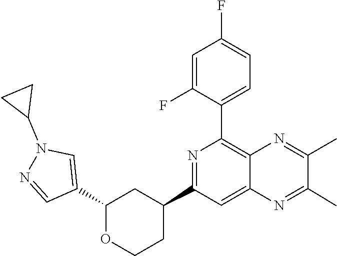 [SMILES: Cc1nc2cc([C@H]3CCO[C@H](c4cnn(C5CC5)c4)C3)nc(-c3ccc(F)cc3F)c2nc1C] |
| I-583 | 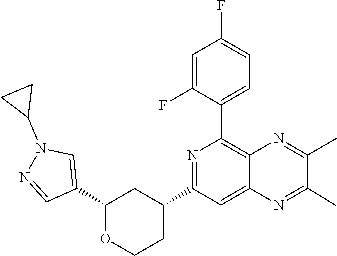 [SMILES: Cc1nc2cc([C@@H]3CCO[C@H](c4cnn(C5CC5)c4)C3)nc(-c3ccc(F)cc3F)c2nc1C] |
| I-584 | 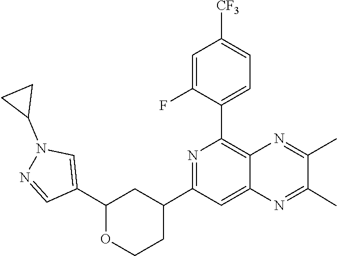 [SMILES: Cc1nc2cc(C3CCOC(c4cnn(C5CC5)c4)C3)nc(-c3ccc(C(F)(F)F)cc3F)c2nc1C] |
| I-585 | 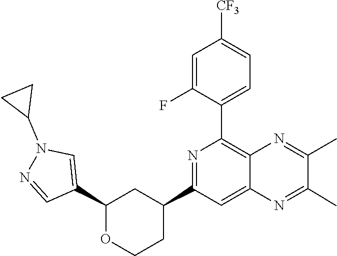 [SMILES: Cc1nc2cc([C@H]3CCO[C@@H](c4cnn(C5CC5)c4)C3)nc(-c3ccc(C(F)(F)F)cc3F)c2nc1C] |
| I-586 | 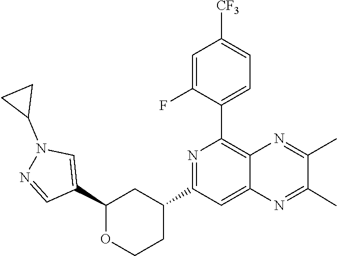 [SMILES: Cc1nc2cc([C@@H]3CCO[C@@H](c4cnn(C5CC5)c4)C3)nc(-c3ccc(C(F)(F)F)cc3F)c2nc1C] |
| I-587 | 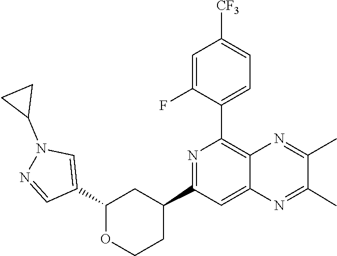 [SMILES: Cc1nc2cc([C@H]3CCO[C@H](c4cnn(C5CC5)c4)C3)nc(-c3ccc(C(F)(F)F)cc3F)c2nc1C] |
| I-588 | 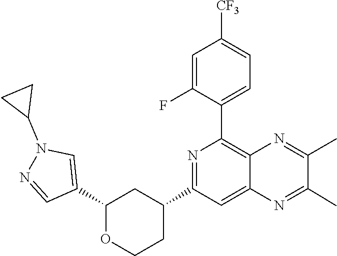 [SMILES: Cc1nc2cc([C@@H]3CCO[C@H](c4cnn(C5CC5)c4)C3)nc(-c3ccc(C(F)(F)F)cc3F)c2nc1C] |
| I-589 | 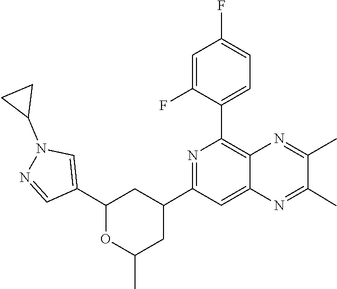 [SMILES: Cc1nc2cc(C3CC(C)OC(c4cnn(C5CC5)c4)C3)nc(-c3ccc(F)cc3F)c2nc1C] |
| I-590 | 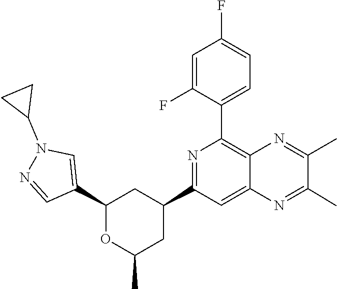 [SMILES: Cc1nc2cc([C@H]3C[C@@H](C)O[C@@H](c4cnn(C5CC5)c4)C3)nc(-c3ccc(F)cc3F)c2nc1C] |
| I-591 | 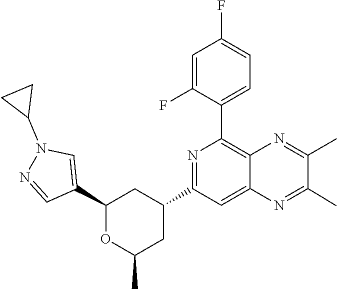 [SMILES: Cc1nc2cc([C@@H]3C[C@@H](C)O[C@@H](c4cnn(C5CC5)c4)C3)nc(-c3ccc(F)cc3F)c2nc1C] |
| I-592 | 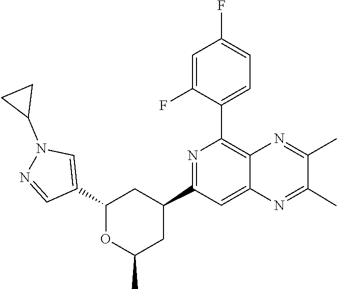 [SMILES: Cc1nc2cc([C@H]3C[C@@H](C)O[C@H](c4cnn(C5CC5)c4)C3)nc(-c3ccc(F)cc3F)c2nc1C] |
| I-593 | 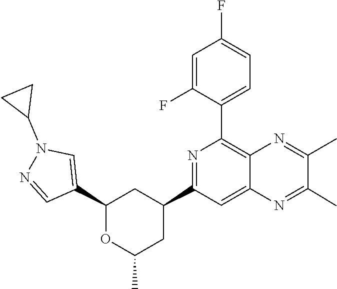 [SMILES: Cc1nc2cc([C@H]3C[C@H](C)O[C@@H](c4cnn(C5CC5)c4)C3)nc(-c3ccc(F)cc3F)c2nc1C] |
| I-594 | 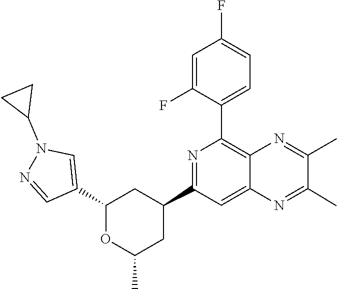 [SMILES: Cc1nc2cc([C@@H]3C[C@@H](c4cnn(C5CC5)c4)O[C@@H](C)C3)nc(-c3ccc(F)cc3F)c2nc1C] |
| I-595 | 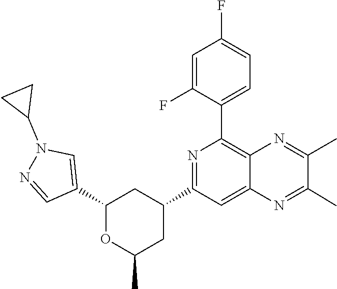 [SMILES: Cc1nc2cc([C@@H]3C[C@@H](C)O[C@H](c4cnn(C5CC5)c4)C3)nc(-c3ccc(F)cc3F)c2nc1C] |
| I-596 | 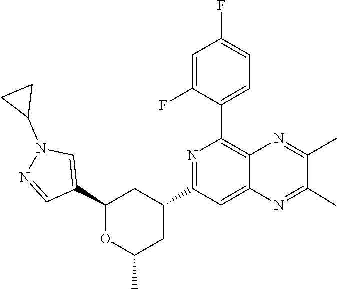 [SMILES: Cc1nc2cc([C@@H]3C[C@H](C)O[C@@H](c4cnn(C5CC5)c4)C3)nc(-c3ccc(F)cc3F)c2nc1C] |
| I-597 | 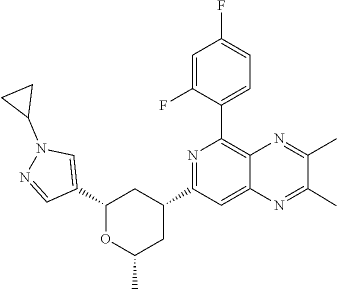 [SMILES: Cc1nc2cc([C@H]3C[C@@H](c4cnn(C5CC5)c4)O[C@@H](C)C3)nc(-c3ccc(F)cc3F)c2nc1C] |
| I-598 | 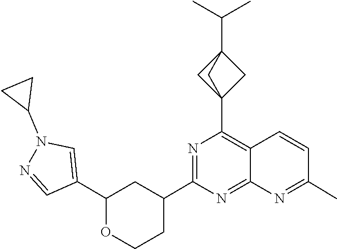 [SMILES: Cc1ccc2c(C34CC(C(C)C)(C3)C4)nc(C3CCOC(c4cnn(C5CC5)c4)C3)nc2n1] |
| I-599 | 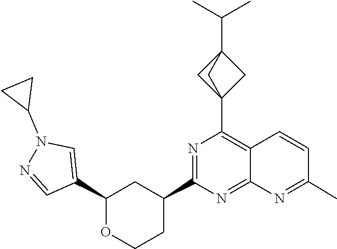 [SMILES: Cc1ccc2c(C34CC(C(C)C)(C3)C4)nc([C@H]3CCO[C@@H](c4cnn(C5CC5)c4)C3)nc2n1] |
| I-600 | 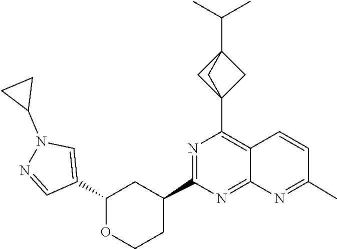 [SMILES: Cc1ccc2c(C34CC(C(C)C)(C3)C4)nc([C@H]3CCO[C@H](c4cnn(C5CC5)c4)C3)nc2n1] |
| I-601 | 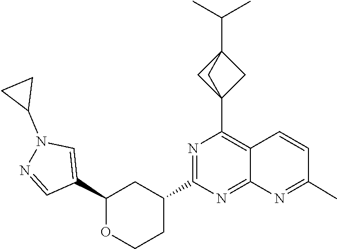 [SMILES: Cc1ccc2c(C34CC(C(C)C)(C3)C4)nc([C@@H]3CCO[C@@H](c4cnn(C5CC5)c4)C3)nc2n1] |
| I-602 | 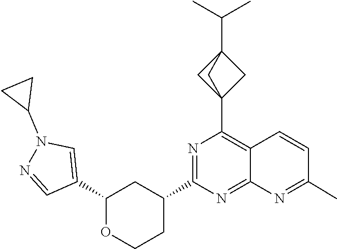 [SMILES: Cc1ccc2c(C34CC(C(C)C)(C3)C4)nc([C@@H]3CCO[C@H](c4cnn(C5CC5)c4)C3)nc2n1] |
| I-603 | 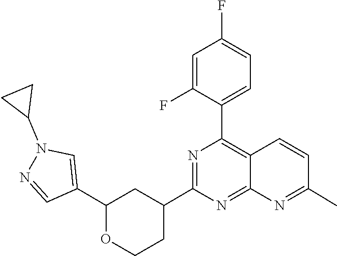 [SMILES: Cc1ccc2c(-c3ccc(F)cc3F)nc(C3CCOC(c4cnn(C5CC5)c4)C3)nc2n1] |
| I-604 | 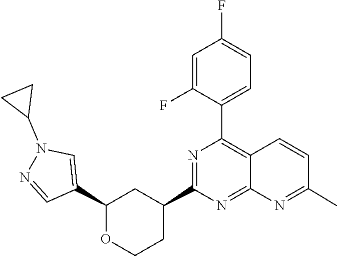 [SMILES: Cc1ccc2c(-c3ccc(F)cc3F)nc([C@H]3CCO[C@@H](c4cnn(C5CC5)c4)C3)nc2n1] |
| I-605 | 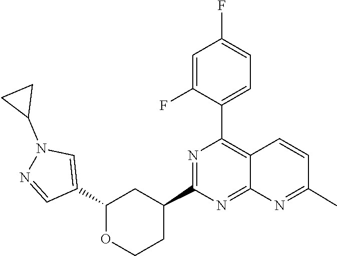 [SMILES: Cc1ccc2c(-c3ccc(F)cc3F)nc([C@H]3CCO[C@H](c4cnn(C5CC5)c4)C3)nc2n1] |
| I-606 | 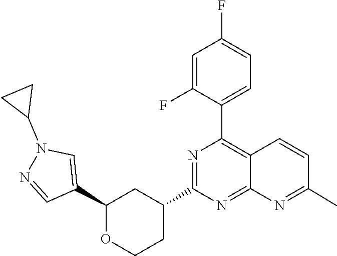 [SMILES: Cc1ccc2c(-c3ccc(F)cc3F)nc([C@@H]3CCO[C@@H](c4cnn(C5CC5)c4)C3)nc2n1] |
| I-607 | 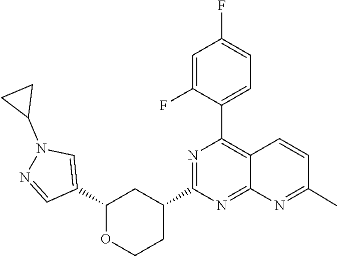 [SMILES: Cc1ccc2c(-c3ccc(F)cc3F)nc([C@@H]3CCO[C@H](c4cnn(C5CC5)c4)C3)nc2n1] |
| I-608 | 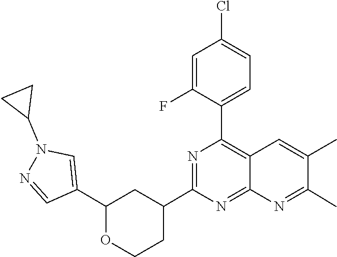 [SMILES: Cc1cc2c(-c3ccc(Cl)cc3F)nc(C3CCOC(c4cnn(C5CC5)c4)C3)nc2nc1C] |
| I-609 | 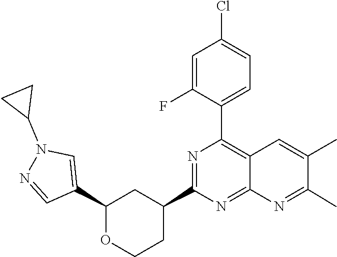 [SMILES: Cc1cc2c(-c3ccc(Cl)cc3F)nc([C@H]3CCO[C@@H](c4cnn(C5CC5)c4)C3)nc2nc1C] |
| I-610 | 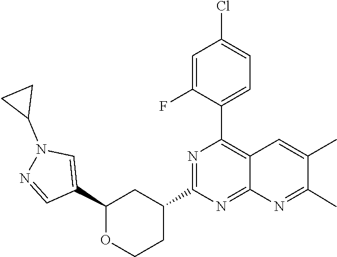 [SMILES: Cc1cc2c(-c3ccc(Cl)cc3F)nc([C@@H]3CCO[C@@H](c4cnn(C5CC5)c4)C3)nc2nc1C] |
| I-611 | 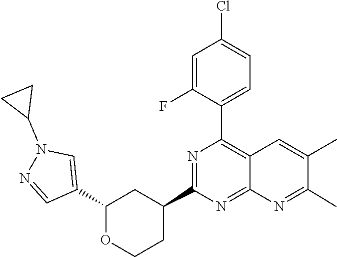 [SMILES: Cc1cc2c(-c3ccc(Cl)cc3F)nc([C@H]3CCO[C@H](c4cnn(C5CC5)c4)C3)nc2nc1C] |
| I-612 | 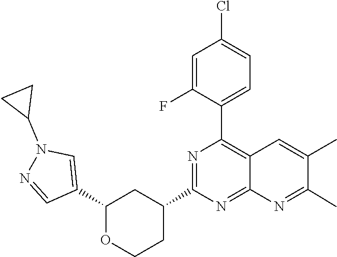 [SMILES: Cc1cc2c(-c3ccc(Cl)cc3F)nc([C@@H]3CCO[C@H](c4cnn(C5CC5)c4)C3)nc2nc1C] |
| I-613 | 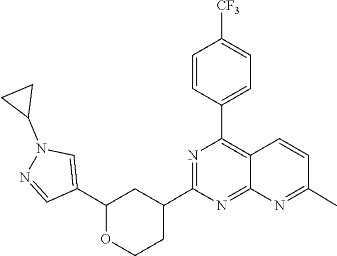 [SMILES: Cc1ccc2c(-c3ccc(C(F)(F)F)cc3)nc(C3CCOC(c4cnn(C5CC5)c4)C3)nc2n1] |
| I-614 | 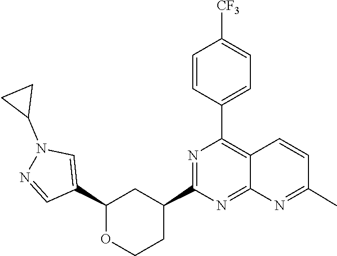 [SMILES: Cc1ccc2c(-c3ccc(C(F)(F)F)cc3)nc([C@H]3CCO[C@@H](c4cnn(C5CC5)c4)C3)nc2n1] |
| I-615 | 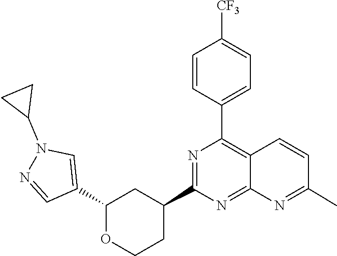 [SMILES: Cc1ccc2c(-c3ccc(C(F)(F)F)cc3)nc([C@H]3CCO[C@H](c4cnn(C5CC5)c4)C3)nc2n1] |
| I-616 | 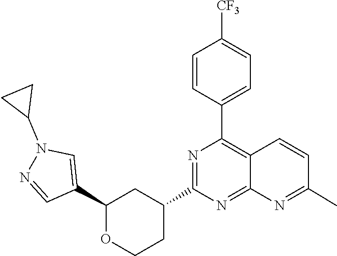 [SMILES: Cc1ccc2c(-c3ccc(C(F)(F)F)cc3)nc([C@@H]3CCO[C@@H](c4cnn(C5CC5)c4)C3)nc2n1] |
| I-617 | 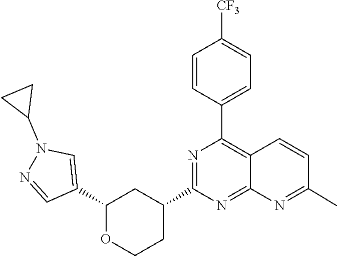 [SMILES: Cc1ccc2c(-c3ccc(C(F)(F)F)cc3)nc([C@@H]3CCO[C@H](c4cnn(C5CC5)c4)C3)nc2n1] |
| I-618 | 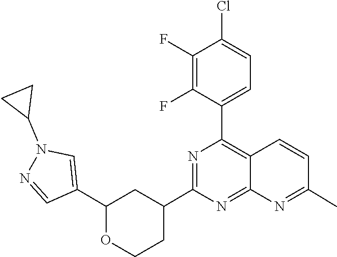 [SMILES: Cc1ccc2c(-c3ccc(Cl)c(F)c3F)nc(C3CCOC(c4cnn(C5CC5)c4)C3)nc2n1] |
| I-619 | 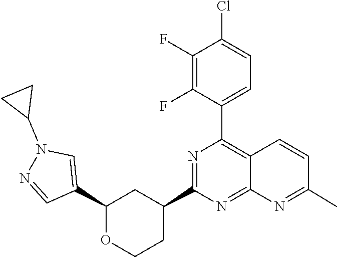 [SMILES: Cc1ccc2c(-c3ccc(Cl)c(F)c3F)nc([C@H]3CCO[C@@H](c4cnn(C5CC5)c4)C3)nc2n1] |
| I-620 | 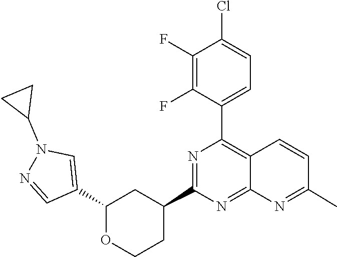 [SMILES: Cc1ccc2c(-c3ccc(Cl)c(F)c3F)nc([C@H]3CCO[C@H](c4cnn(C5CC5)c4)C3)nc2n1] |
| I-621 | 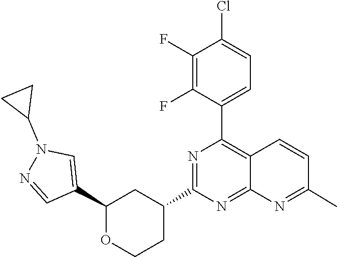 [SMILES: Cc1ccc2c(-c3ccc(Cl)c(F)c3F)nc([C@@H]3CCO[C@@H](c4cnn(C5CC5)c4)C3)nc2n1] |
| I-622 | 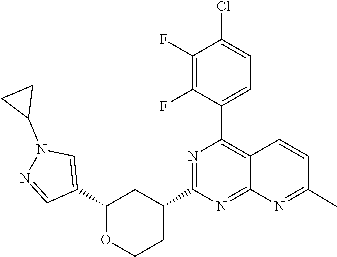 [SMILES: Cc1ccc2c(-c3ccc(Cl)c(F)c3F)nc([C@@H]3CCO[C@H](c4cnn(C5CC5)c4)C3)nc2n1] |
| I-623 | 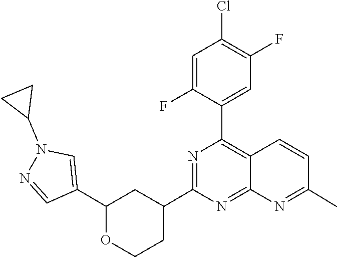 [SMILES: Cc1ccc2c(-c3cc(F)c(Cl)cc3F)nc(C3CCOC(c4cnn(C5CC5)c4)C3)nc2n1] |
| I-624 | 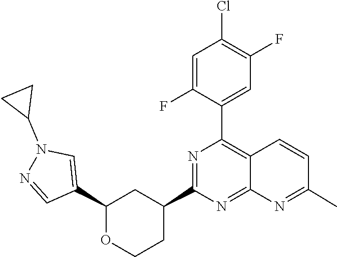 [SMILES: Cc1ccc2c(-c3cc(F)c(Cl)cc3F)nc([C@H]3CCO[C@@H](c4cnn(C5CC5)c4)C3)nc2n1] |
| I-625 | 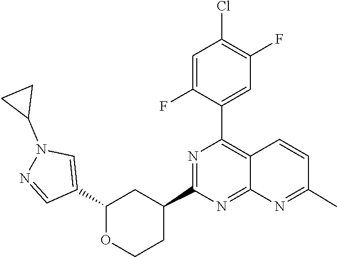 [SMILES: Cc1ccc2c(-c3cc(F)c(Cl)cc3F)nc([C@H]3CCO[C@H](c4cnn(C5CC5)c4)C3)nc2n1] |
| I-626 | 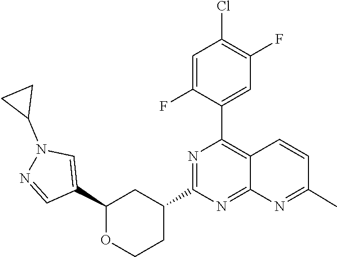 [SMILES: Cc1ccc2c(-c3cc(F)c(Cl)cc3F)nc([C@@H]3CCO[C@@H](c4cnn(C5CC5)c4)C3)nc2n1] |
| I-627 | 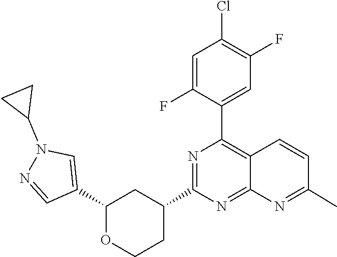 [SMILES: Cc1ccc2c(-c3cc(F)c(Cl)cc3F)nc([C@@H]3CCO[C@H](c4cnn(C5CC5)c4)C3)nc2n1] |
| I-628 | 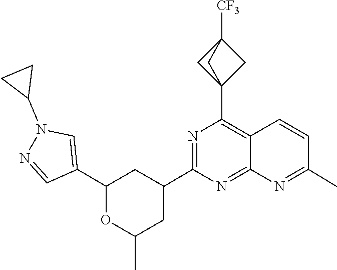 [SMILES: Cc1ccc2c(C34CC(C(F)(F)F)(C3)C4)nc(C3CC(C)OC(c4cnn(C5CC5)c4)C3)nc2n1] |
| I-629 | 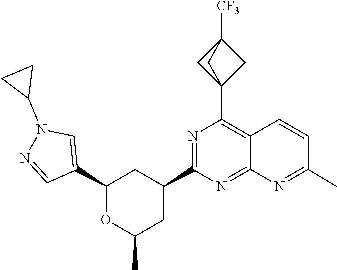 [SMILES: Cc1ccc2c(C34CC(C(F)(F)F)(C3)C4)nc([C@H]3C[C@@H](C)O[C@@H](c4cnn(C5CC5)c4)C3)nc2n1] |
| I-630 | 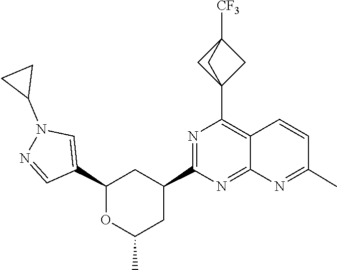 [SMILES: Cc1ccc2c(C34CC(C(F)(F)F)(C3)C4)nc([C@H]3C[C@H](C)O[C@@H](c4cnn(C5CC5)c4)C3)nc2n1] |
| I-631 | 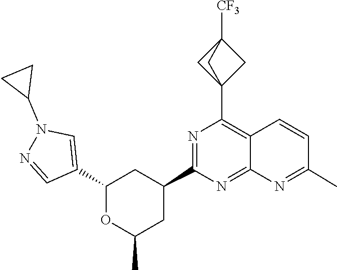 [SMILES: Cc1ccc2c(C34CC(C(F)(F)F)(C3)C4)nc([C@H]3C[C@@H](C)O[C@H](c4cnn(C5CC5)c4)C3)nc2n1] |
| I-632 | 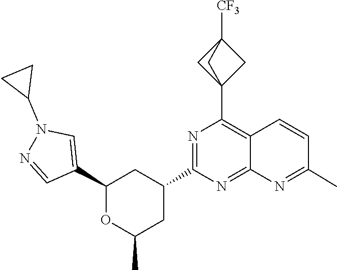 [SMILES: Cc1ccc2c(C34CC(C(F)(F)F)(C3)C4)nc([C@@H]3C[C@@H](C)O[C@@H](c4cnn(C5CC5)c4)C3)nc2n1] |
| I-633 | 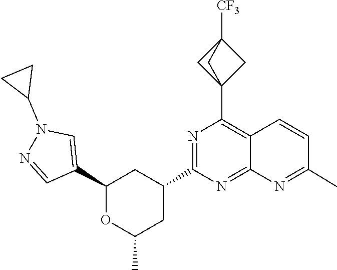 [SMILES: Cc1ccc2c(C34CC(C(F)(F)F)(C3)C4)nc([C@@H]3C[C@H](C)O[C@@H](c4cnn(C5CC5)c4)C3)nc2n1] |
| I-634 | 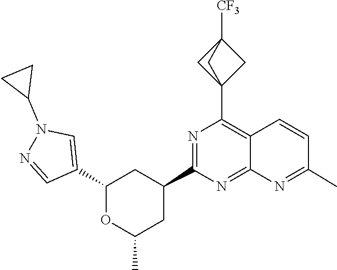 [SMILES: Cc1ccc2c(C34CC(C(F)(F)F)(C3)C4)nc([C@@H]3C[C@@H](c4cnn(C5CC5)c4)O[C@@H](C)C3)nc2n1] |
| I-635 | 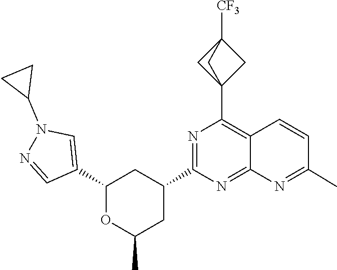 [SMILES: Cc1ccc2c(C34CC(C(F)(F)F)(C3)C4)nc([C@@H]3C[C@@H](C)O[C@H](c4cnn(C5CC5)c4)C3)nc2n1] |
| I-636 | 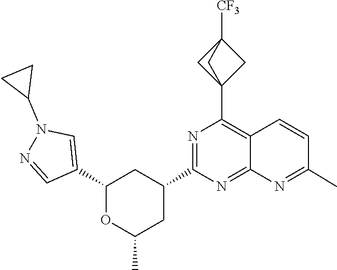 [SMILES: Cc1ccc2c(C34CC(C(F)(F)F)(C3)C4)nc([C@H]3C[C@@H](c4cnn(C5CC5)c4)O[C@@H](C)C3)nc2n1] |
| I-637 | 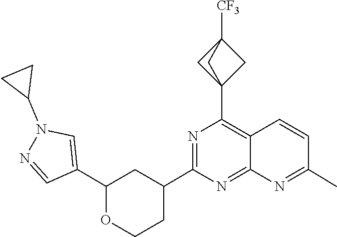 [SMILES: Cc1ccc2c(C34CC(C(F)(F)F)(C3)C4)nc(C3CCOC(c4cnn(C5CC5)c4)C3)nc2n1] |
| I-638 | 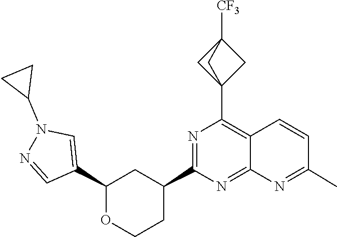 [SMILES: Cc1ccc2c(C34CC(C(F)(F)F)(C3)C4)nc([C@H]3CCO[C@@H](c4cnn(C5CC5)c4)C3)nc2n1] |
| I-639 | 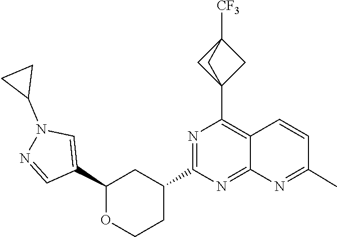 [SMILES: Cc1ccc2c(C34CC(C(F)(F)F)(C3)C4)nc([C@@H]3CCO[C@@H](c4cnn(C5CC5)c4)C3)nc2n1] |
| I-640 | 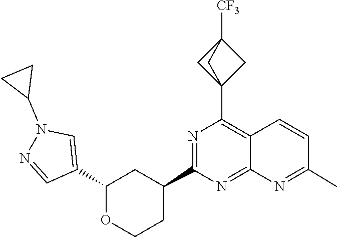 [SMILES: Cc1ccc2c(C34CC(C(F)(F)F)(C3)C4)nc([C@H]3CCO[C@H](c4cnn(C5CC5)c4)C3)nc2n1] |
| I-641 | 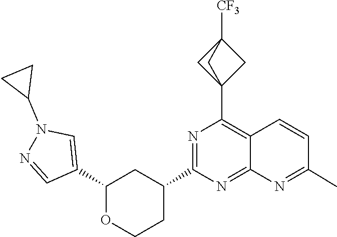 [SMILES: Cc1ccc2c(C34CC(C(F)(F)F)(C3)C4)nc([C@@H]3CCO[C@H](c4cnn(C5CC5)c4)C3)nc2n1] |
| I-642 | 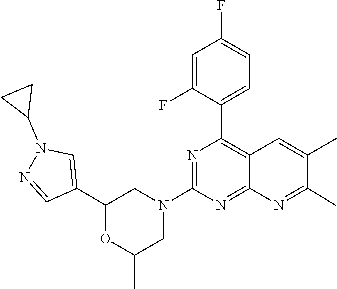 [SMILES: Cc1cc2c(-c3ccc(F)cc3F)nc(N3CC(C)OC(c4cnn(C5CC5)c4)C3)nc2nc1C] |
| I-643 | 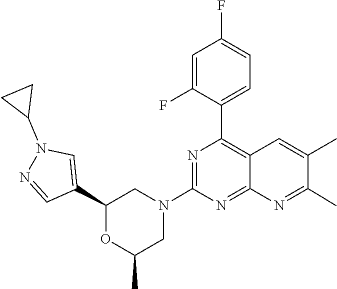 [SMILES: Cc1cc2c(-c3ccc(F)cc3F)nc(N3C[C@@H](C)O[C@@H](c4cnn(C5CC5)c4)C3)nc2nc1C] |
| I-644 | 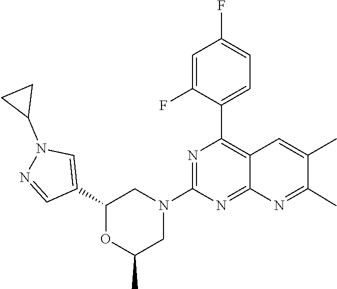 [SMILES: Cc1cc2c(-c3ccc(F)cc3F)nc(N3C[C@@H](C)O[C@H](c4cnn(C5CC5)c4)C3)nc2nc1C] |
| I-645 | 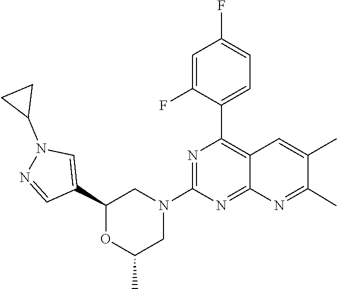 [SMILES: Cc1cc2c(-c3ccc(F)cc3F)nc(N3C[C@H](C)O[C@@H](c4cnn(C5CC5)c4)C3)nc2nc1C] |
| I-646 | 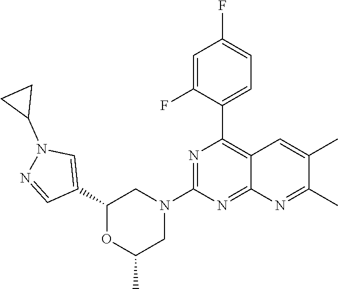 [SMILES: Cc1cc2c(-c3ccc(F)cc3F)nc(N3C[C@@H](c4cnn(C5CC5)c4)O[C@@H](C)C3)nc2nc1C] |
| I-647 | 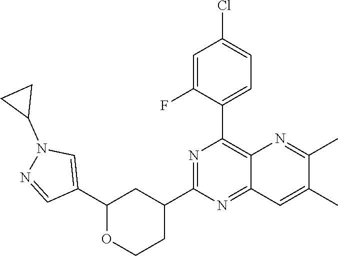 [SMILES: Cc1cc2nc(C3CCOC(c4cnn(C5CC5)c4)C3)nc(-c3ccc(Cl)cc3F)c2nc1C] |
| I-648 | 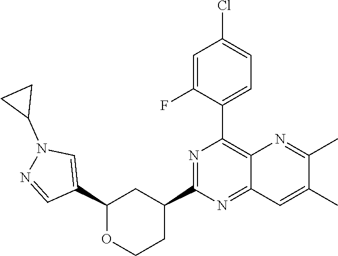 [SMILES: Cc1cc2nc([C@H]3CCO[C@@H](c4cnn(C5CC5)c4)C3)nc(-c3ccc(Cl)cc3F)c2nc1C] |
| I-649 | 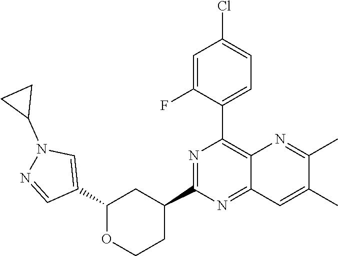 [SMILES: Cc1cc2nc([C@H]3CCO[C@H](c4cnn(C5CC5)c4)C3)nc(-c3ccc(Cl)cc3F)c2nc1C] |
| I-650 | 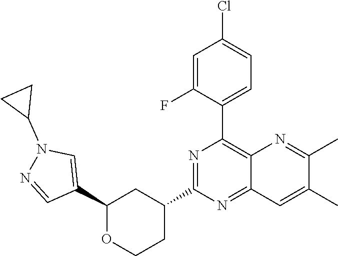 [SMILES: Cc1cc2nc([C@@H]3CCO[C@@H](c4cnn(C5CC5)c4)C3)nc(-c3ccc(Cl)cc3F)c2nc1C] |
| I-651 | 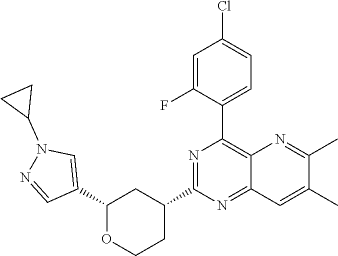 [SMILES: Cc1cc2nc([C@@H]3CCO[C@H](c4cnn(C5CC5)c4)C3)nc(-c3ccc(Cl)cc3F)c2nc1C] |
| I-652 | 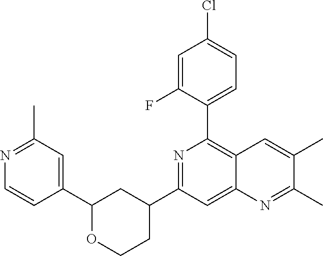 [SMILES: Cc1cc(C2CC(c3cc4nc(C)c(C)cc4c(-c4ccc(Cl)cc4F)n3)CCO2)ccn1] |
| I-653 | 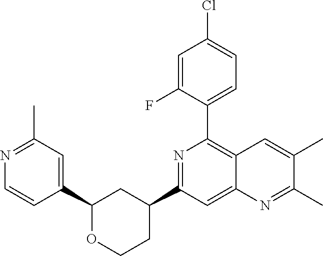 [SMILES: Cc1cc([C@H]2C[C@@H](c3cc4nc(C)c(C)cc4c(-c4ccc(Cl)cc4F)n3)CCO2)ccn1] |
| I-654 | 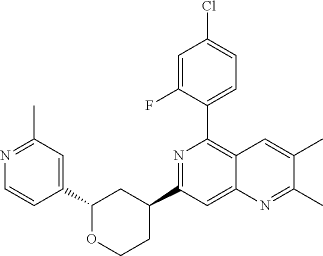 [SMILES: Cc1cc([C@@H]2C[C@@H](c3cc4nc(C)c(C)cc4c(-c4ccc(Cl)cc4F)n3)CCO2)ccn1] |
| I-655 | 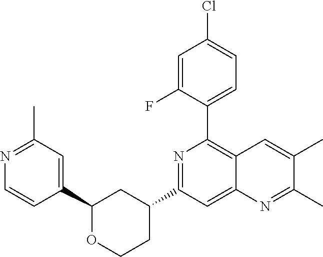 [SMILES: Cc1cc([C@H]2C[C@H](c3cc4nc(C)c(C)cc4c(-c4ccc(Cl)cc4F)n3)CCO2)ccn1] |
| I-656 | 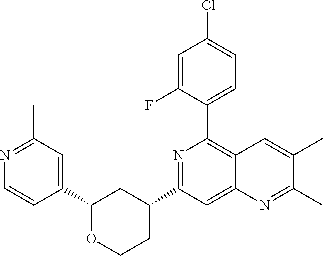 [SMILES: Cc1cc([C@@H]2C[C@H](c3cc4nc(C)c(C)cc4c(-c4ccc(Cl)cc4F)n3)CCO2)ccn1] |
| I-657 | 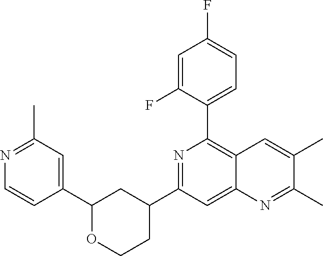 [SMILES: Cc1cc(C2CC(c3cc4nc(C)c(C)cc4c(-c4ccc(F)cc4F)n3)CCO2)ccn1] |
| I-658 | 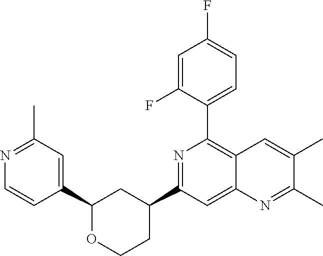 [SMILES: Cc1cc([C@H]2C[C@@H](c3cc4nc(C)c(C)cc4c(-c4ccc(F)cc4F)n3)CCO2)ccn1] |
| I-659 | 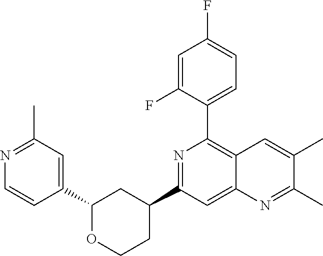 [SMILES: Cc1cc([C@@H]2C[C@@H](c3cc4nc(C)c(C)cc4c(-c4ccc(F)cc4F)n3)CCO2)ccn1] |
| I-660 | 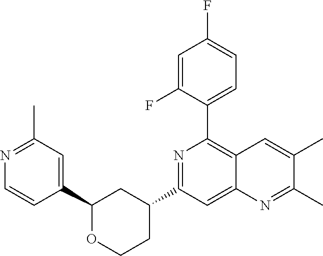 [SMILES: Cc1cc([C@H]2C[C@H](c3cc4nc(C)c(C)cc4c(-c4ccc(F)cc4F)n3)CCO2)ccn1] |
| I-661 | 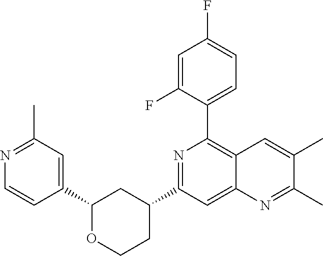 [SMILES: Cc1cc([C@@H]2C[C@H](c3cc4nc(C)c(C)cc4c(-c4ccc(F)cc4F)n3)CCO2)ccn1] |
| I-662 | 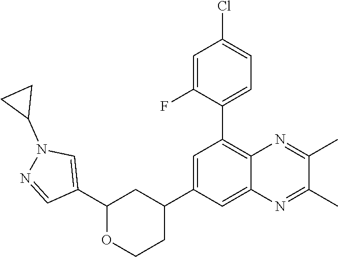 [SMILES: Cc1nc2cc(C3CCOC(c4cnn(C5CC5)c4)C3)cc(-c3ccc(Cl)cc3F)c2nc1C] |
| I-663 | 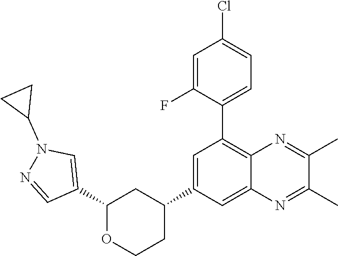 [SMILES: Cc1nc2cc([C@@H]3CCO[C@H](c4cnn(C5CC5)c4)C3)cc(-c3ccc(Cl)cc3F)c2nc1C] |
| I-664 | 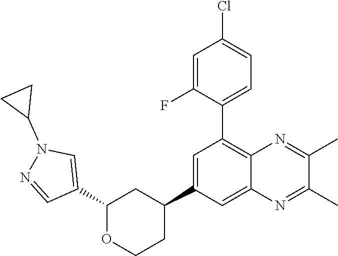 [SMILES: Cc1nc2cc([C@H]3CCO[C@H](c4cnn(C5CC5)c4)C3)cc(-c3ccc(Cl)cc3F)c2nc1C] |
| I-665 | 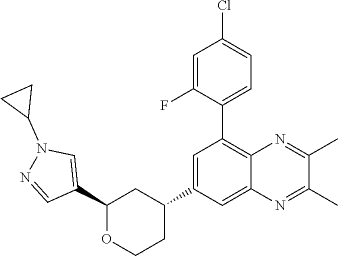 [SMILES: Cc1nc2cc([C@@H]3CCO[C@@H](c4cnn(C5CC5)c4)C3)cc(-c3ccc(Cl)cc3F)c2nc1C] |
| I-666 | 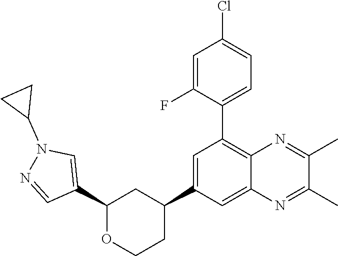 [SMILES: Cc1nc2cc([C@H]3CCO[C@@H](c4cnn(C5CC5)c4)C3)cc(-c3ccc(Cl)cc3F)c2nc1C] |
| I-667 | 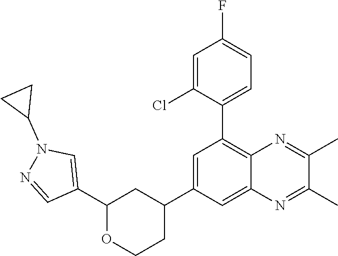 [SMILES: Cc1nc2cc(C3CCOC(c4cnn(C5CC5)c4)C3)cc(-c3ccc(F)cc3Cl)c2nc1C] |
| I-668 | 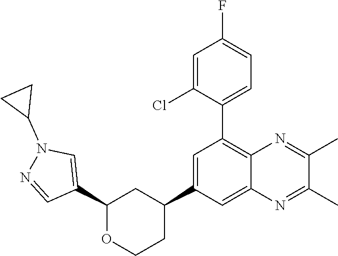 [SMILES: Cc1nc2cc([C@H]3CCO[C@@H](c4cnn(C5CC5)c4)C3)cc(-c3ccc(F)cc3Cl)c2nc1C] |
| I-669 | 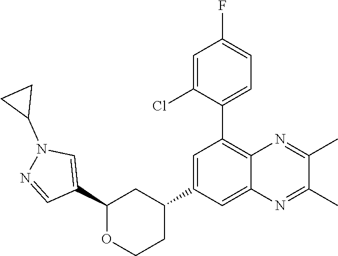 [SMILES: Cc1nc2cc([C@@H]3CCO[C@@H](c4cnn(C5CC5)c4)C3)cc(-c3ccc(F)cc3Cl)c2nc1C] |
| I-670 | 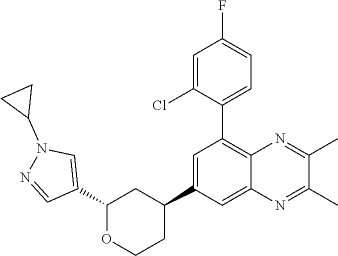 [SMILES: Cc1nc2cc([C@H]3CCO[C@H](c4cnn(C5CC5)c4)C3)cc(-c3ccc(F)cc3Cl)c2nc1C] |
| I-671 | 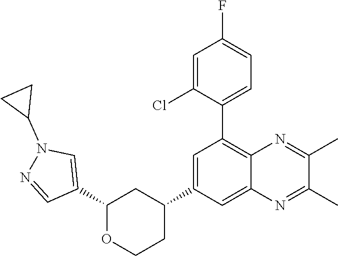 [SMILES: Cc1nc2cc([C@@H]3CCO[C@H](c4cnn(C5CC5)c4)C3)cc(-c3ccc(F)cc3Cl)c2nc1C] |
| I-672 | 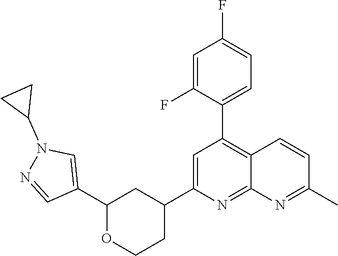 [SMILES: Cc1ccc2c(-c3ccc(F)cc3F)cc(C3CCOC(c4cnn(C5CC5)c4)C3)nc2n1] |
| I-673 | 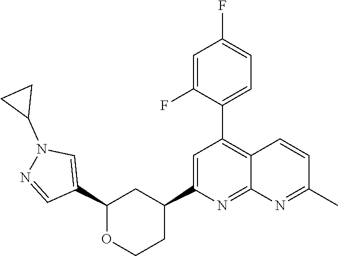 [SMILES: Cc1ccc2c(-c3ccc(F)cc3F)cc([C@H]3CCO[C@@H](c4cnn(C5CC5)c4)C3)nc2n1] |
| I-674 | 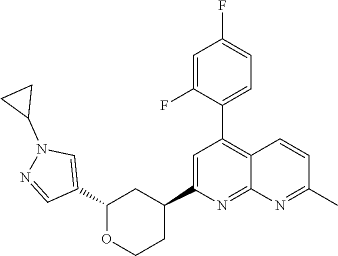 [SMILES: Cc1ccc2c(-c3ccc(F)cc3F)cc([C@H]3CCO[C@H](c4cnn(C5CC5)c4)C3)nc2n1] |
| I-675 | 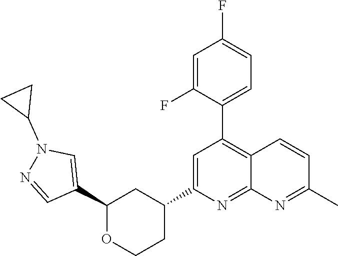 [SMILES: Cc1ccc2c(-c3ccc(F)cc3F)cc([C@@H]3CCO[C@@H](c4cnn(C5CC5)c4)C3)nc2n1] |
| I-676 | 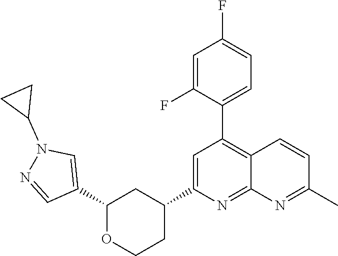 [SMILES: Cc1ccc2c(-c3ccc(F)cc3F)cc([C@@H]3CCO[C@H](c4cnn(C5CC5)c4)C3)nc2n1] |
| I-677 | 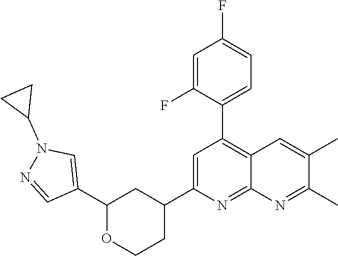 [SMILES: Cc1cc2c(-c3ccc(F)cc3F)cc(C3CCOC(c4cnn(C5CC5)c4)C3)nc2nc1C] |
| I-678 | 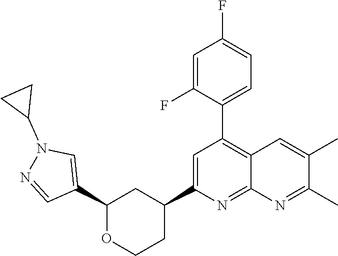 [SMILES: Cc1cc2c(-c3ccc(F)cc3F)cc([C@H]3CCO[C@@H](c4cnn(C5CC5)c4)C3)nc2nc1C] |
| I-679 | 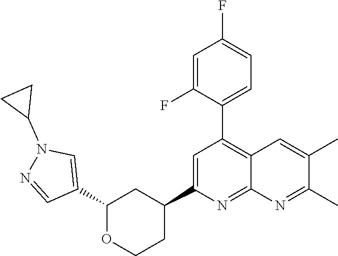 [SMILES: Cc1cc2c(-c3ccc(F)cc3F)cc([C@H]3CCO[C@H](c4cnn(C5CC5)c4)C3)nc2nc1C] |
| I-680 | 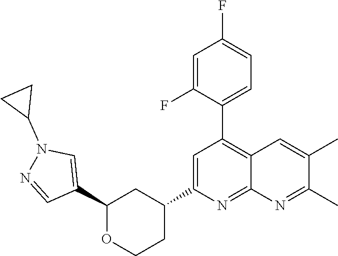 [SMILES: Cc1cc2c(-c3ccc(F)cc3F)cc([C@@H]3CCO[C@@H](c4cnn(C5CC5)c4)C3)nc2nc1C] |
| I-681 | 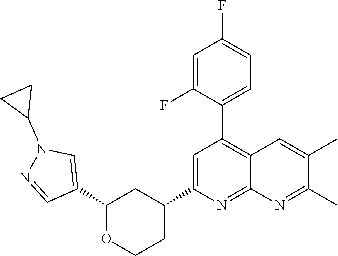 [SMILES: Cc1cc2c(-c3ccc(F)cc3F)cc([C@@H]3CCO[C@H](c4cnn(C5CC5)c4)C3)nc2nc1C] |
| I-682 | 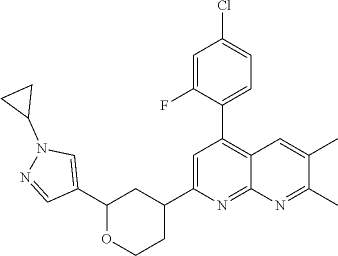 [SMILES: Cc1cc2c(-c3ccc(Cl)cc3F)cc(C3CCOC(c4cnn(C5CC5)c4)C3)nc2nc1C] |
| I-683 | 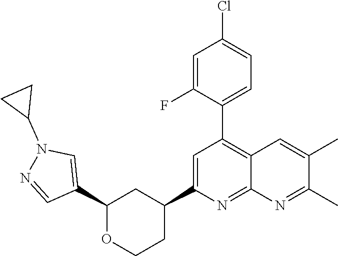 [SMILES: Cc1cc2c(-c3ccc(Cl)cc3F)cc([C@H]3CCO[C@@H](c4cnn(C5CC5)c4)C3)nc2nc1C] |
| I-684 | 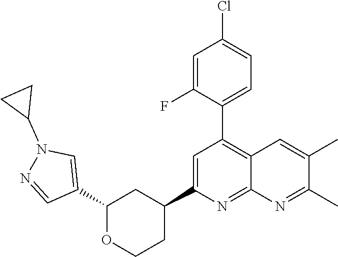 [SMILES: Cc1cc2c(-c3ccc(Cl)cc3F)cc([C@H]3CCO[C@H](c4cnn(C5CC5)c4)C3)nc2nc1C] |
| I-685 | 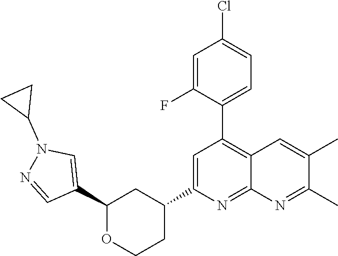 [SMILES: Cc1cc2c(-c3ccc(Cl)cc3F)cc([C@@H]3CCO[C@@H](c4cnn(C5CC5)c4)C3)nc2nc1C] |
| I-686 | 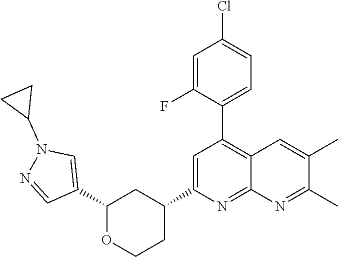 [SMILES: Cc1cc2c(-c3ccc(Cl)cc3F)cc([C@@H]3CCO[C@H](c4cnn(C5CC5)c4)C3)nc2nc1C] |
| I-687 | 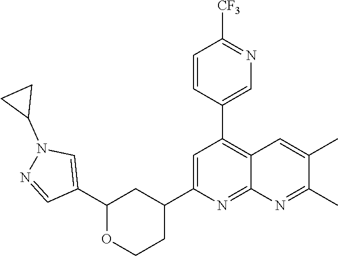 [SMILES: Cc1cc2c(-c3ccc(C(F)(F)F)nc3)cc(C3CCOC(c4cnn(C5CC5)c4)C3)nc2nc1C] |
| I-688 | 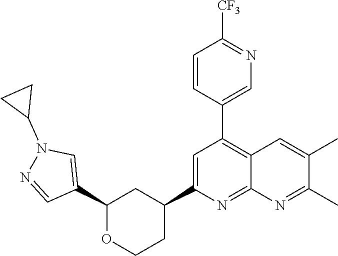 [SMILES: Cc1cc2c(-c3ccc(C(F)(F)F)nc3)cc([C@H]3CCO[C@@H](c4cnn(C5CC5)c4)C3)nc2nc1C] |
| I-689 | 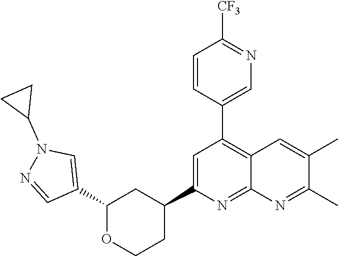 [SMILES: Cc1cc2c(-c3ccc(C(F)(F)F)nc3)cc([C@H]3CCO[C@H](c4cnn(C5CC5)c4)C3)nc2nc1C] |
| I-690 | 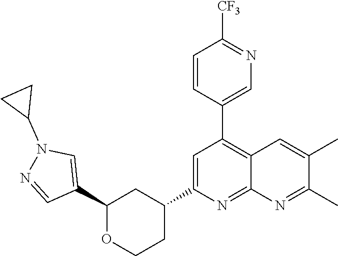 [SMILES: Cc1cc2c(-c3ccc(C(F)(F)F)nc3)cc([C@@H]3CCO[C@@H](c4cnn(C5CC5)c4)C3)nc2nc1C] |
| I-691 | 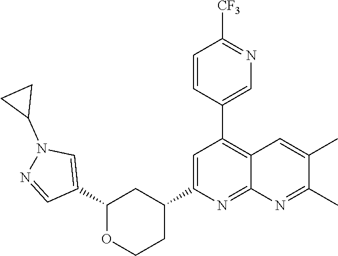 [SMILES: Cc1cc2c(-c3ccc(C(F)(F)F)nc3)cc([C@@H]3CCO[C@H](c4cnn(C5CC5)c4)C3)nc2nc1C] |
| I-692 | 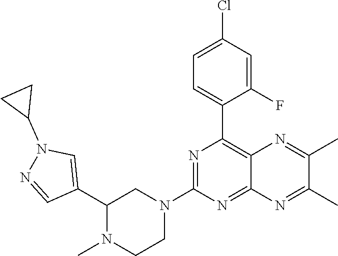 [SMILES: Cc1nc2nc(N3CCN(C)C(c4cnn(C5CC5)c4)C3)nc(-c3ccc(Cl)cc3F)c2nc1C] |
| I-693 | 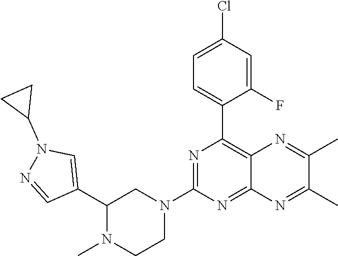 [SMILES: Cc1nc2nc(N3CCN(C)C(c4cnn(C5CC5)c4)C3)nc(-c3ccc(Cl)cc3F)c2nc1C] |
| I-694 | 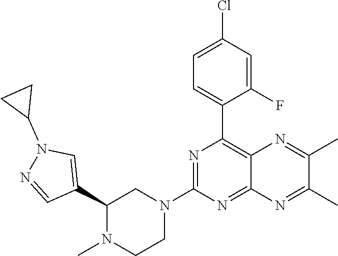 [SMILES: Cc1nc2nc(N3CCN(C)[C@@H](c4cnn(C5CC5)c4)C3)nc(-c3ccc(Cl)cc3F)c2nc1C] |
| I-695 | 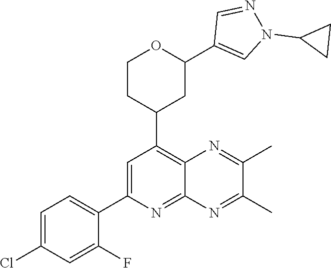 [SMILES: Cc1nc2nc(-c3ccc(Cl)cc3F)cc(C3CCOC(c4cnn(C5CC5)c4)C3)c2nc1C] |
| I-696 | 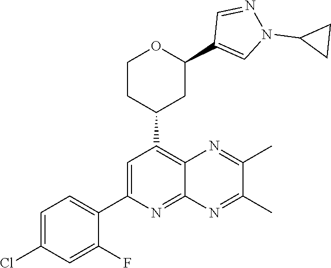 [SMILES: Cc1nc2nc(-c3ccc(Cl)cc3F)cc([C@@H]3CCO[C@@H](c4cnn(C5CC5)c4)C3)c2nc1C] |
| I-697 | 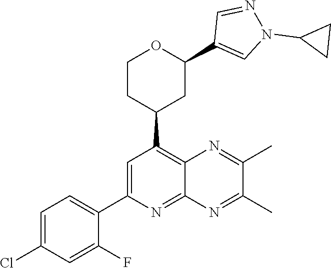 [SMILES: Cc1nc2nc(-c3ccc(Cl)cc3F)cc([C@H]3CCO[C@@H](c4cnn(C5CC5)c4)C3)c2nc1C] |
| I-698 | 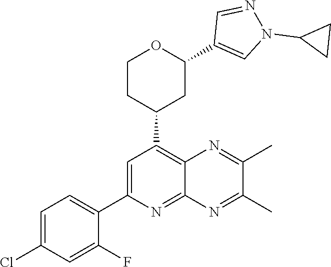 [SMILES: Cc1nc2nc(-c3ccc(Cl)cc3F)cc([C@@H]3CCO[C@H](c4cnn(C5CC5)c4)C3)c2nc1C] |
| I-699 | 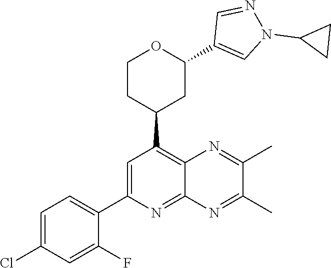 [SMILES: Cc1nc2nc(-c3ccc(Cl)cc3F)cc([C@H]3CCO[C@H](c4cnn(C5CC5)c4)C3)c2nc1C] |
| I-600 | 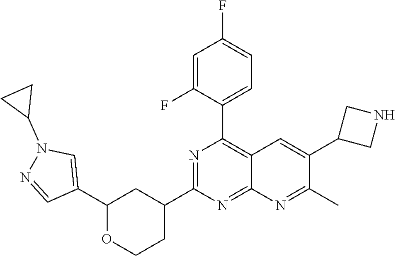 [SMILES: Cc1nc2nc(C3CCOC(c4cnn(C5CC5)c4)C3)nc(-c3ccc(F)cc3F)c2cc1C1CNC1] |
| I-601 | 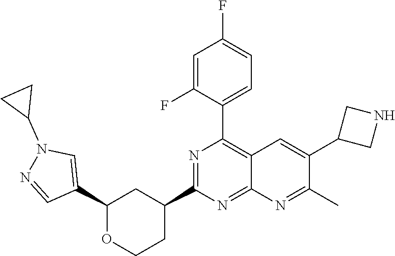 [SMILES: Cc1nc2nc([C@H]3CCO[C@@H](c4cnn(C5CC5)c4)C3)nc(-c3ccc(F)cc3F)c2cc1C1CNC1] |
| I-602 | 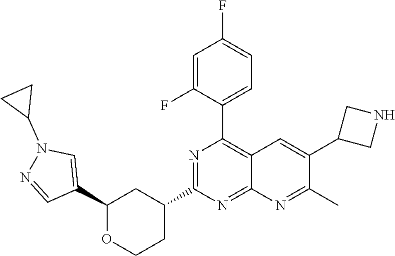 [SMILES: Cc1nc2nc([C@@H]3CCO[C@@H](c4cnn(C5CC5)c4)C3)nc(-c3ccc(F)cc3F)c2cc1C1CNC1] |
| I-603 | 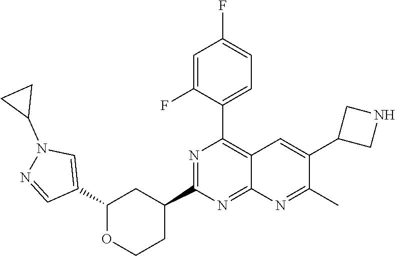 [SMILES: Cc1nc2nc([C@H]3CCO[C@H](c4cnn(C5CC5)c4)C3)nc(-c3ccc(F)cc3F)c2cc1C1CNC1] |
| I-604 | 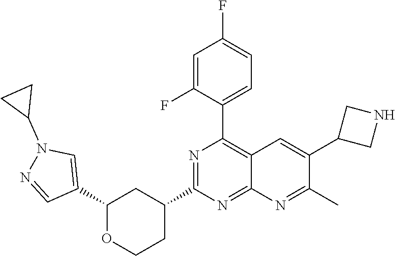 [SMILES: Cc1nc2nc([C@@H]3CCO[C@H](c4cnn(C5CC5)c4)C3)nc(-c3ccc(F)cc3F)c2cc1C1CNC1] |
| I-605 | 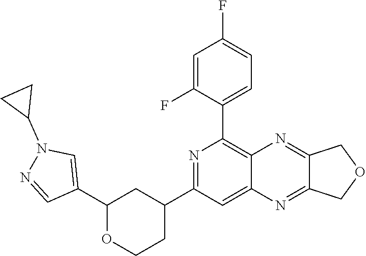 [SMILES: Fc1ccc(-c2nc(C3CCOC(c4cnn(C5CC5)c4)C3)cc3nc4c(nc23)COC4)c(F)c1] |
| I-606 | 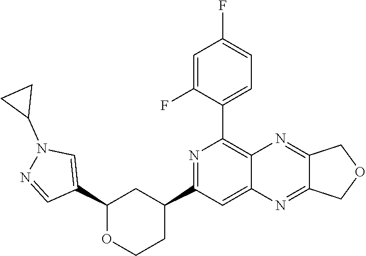 [SMILES: Fc1ccc(-c2nc([C@H]3CCO[C@@H](c4cnn(C5CC5)c4)C3)cc3nc4c(nc23)COC4)c(F)c1] |
| I-607 | 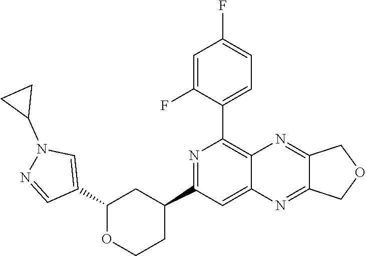 [SMILES: Fc1ccc(-c2nc([C@H]3CCO[C@H](c4cnn(C5CC5)c4)C3)cc3nc4c(nc23)COC4)c(F)c1] |
| I-608 | 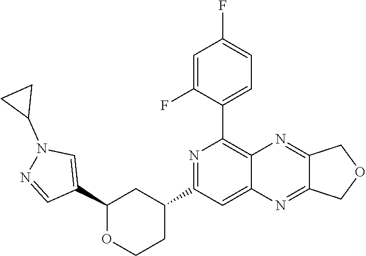 [SMILES: Fc1ccc(-c2nc([C@@H]3CCO[C@@H](c4cnn(C5CC5)c4)C3)cc3nc4c(nc23)COC4)c(F)c1] |
| I-609 | 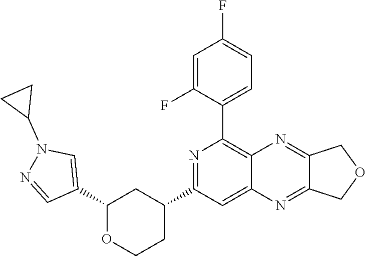 [SMILES: Fc1ccc(-c2nc([C@@H]3CCO[C@H](c4cnn(C5CC5)c4)C3)cc3nc4c(nc23)COC4)c(F)c1] |
| I-610 | 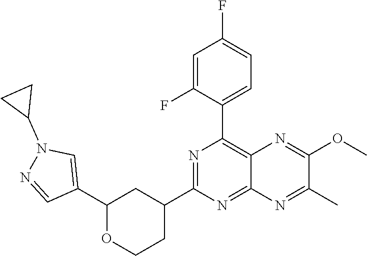 [SMILES: COc1nc2c(-c3ccc(F)cc3F)nc(C3CCOC(c4cnn(C5CC5)c4)C3)nc2nc1C] |
| I-611 | 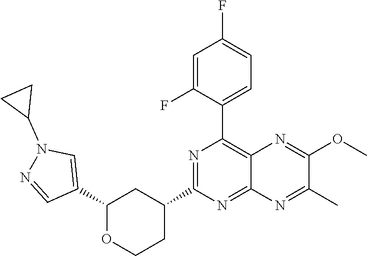 [SMILES: COc1nc2c(-c3ccc(F)cc3F)nc([C@@H]3CCO[C@H](c4cnn(C5CC5)c4)C3)nc2nc1C] |
| I-612 | 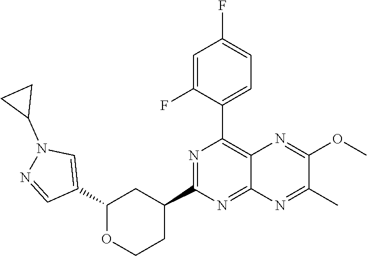 [SMILES: COc1nc2c(-c3ccc(F)cc3F)nc([C@H]3CCO[C@H](c4cnn(C5CC5)c4)C3)nc2nc1C] |
| I-613 | 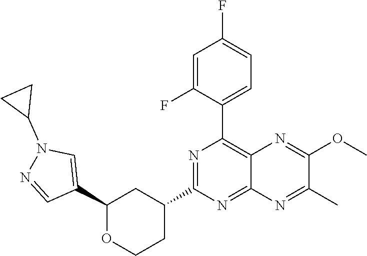 [SMILES: COc1nc2c(-c3ccc(F)cc3F)nc([C@@H]3CCO[C@@H](c4cnn(C5CC5)c4)C3)nc2nc1C] |
| I-614 | 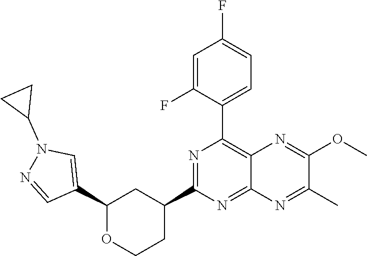 [SMILES: COc1nc2c(-c3ccc(F)cc3F)nc([C@H]3CCO[C@@H](c4cnn(C5CC5)c4)C3)nc2nc1C] |
| I-615 | 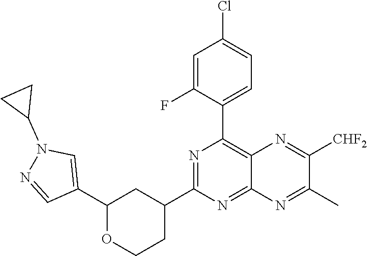 [SMILES: Cc1nc2nc(C3CCOC(c4cnn(C5CC5)c4)C3)nc(-c3ccc(Cl)cc3F)c2nc1C(F)F] |
| I-616 | 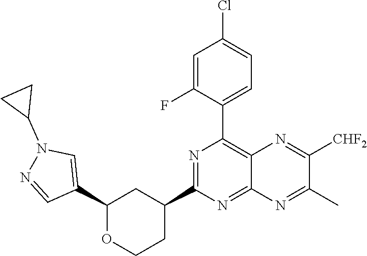 [SMILES: Cc1nc2nc([C@H]3CCO[C@@H](c4cnn(C5CC5)c4)C3)nc(-c3ccc(Cl)cc3F)c2nc1C(F)F] |
| I-617 | 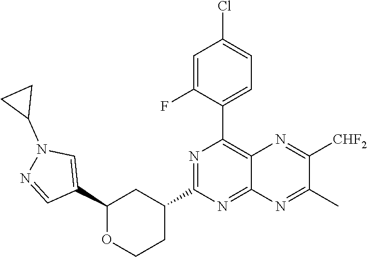 [SMILES: Cc1nc2nc([C@@H]3CCO[C@@H](c4cnn(C5CC5)c4)C3)nc(-c3ccc(Cl)cc3F)c2nc1C(F)F] |
| I-618 | 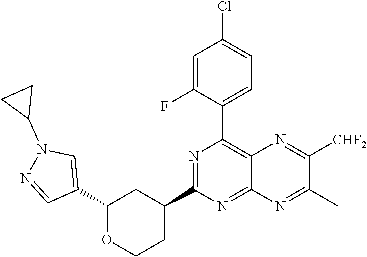 [SMILES: Cc1nc2nc([C@H]3CCO[C@H](c4cnn(C5CC5)c4)C3)nc(-c3ccc(Cl)cc3F)c2nc1C(F)F] |
| I-619 | 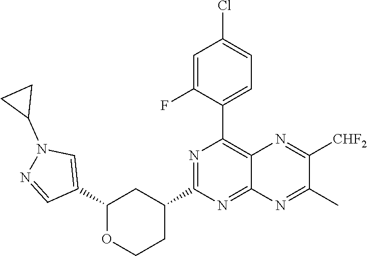 [SMILES: Cc1nc2nc([C@@H]3CCO[C@H](c4cnn(C5CC5)c4)C3)nc(-c3ccc(Cl)cc3F)c2nc1C(F)F] |
| I-620 | 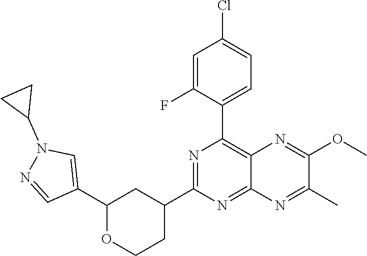 [SMILES: COc1nc2c(-c3ccc(Cl)cc3F)nc(C3CCOC(c4cnn(C5CC5)c4)C3)nc2nc1C] |
| I-621 | 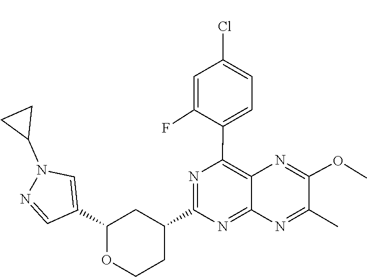 |
| I-622 | 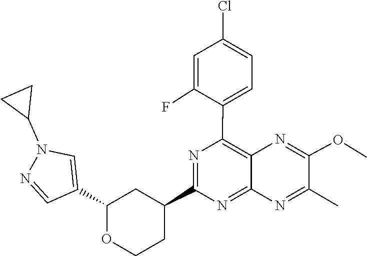 [SMILES: COc1nc2c(-c3ccc(Cl)cc3F)nc([C@H]3CCO[C@H](c4cnn(C5CC5)c4)C3)nc2nc1C] |
| I-623 | 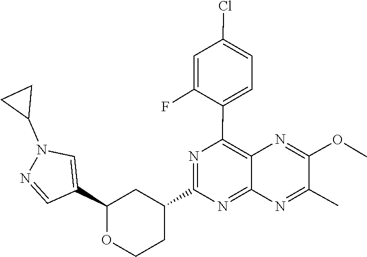 [SMILES: COc1nc2c(-c3ccc(Cl)cc3F)nc([C@@H]3CCO[C@@H](c4cnn(C5CC5)c4)C3)nc2nc1C] |
| I-624 | 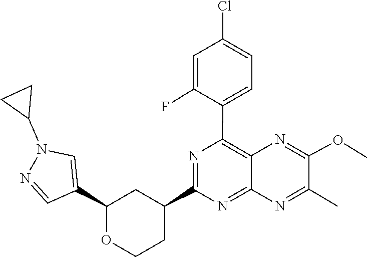 [SMILES: COc1nc2c(-c3ccc(Cl)cc3F)nc([C@H]3CCO[C@@H](c4cnn(C5CC5)c4)C3)nc2nc1C] |
| I-625 | 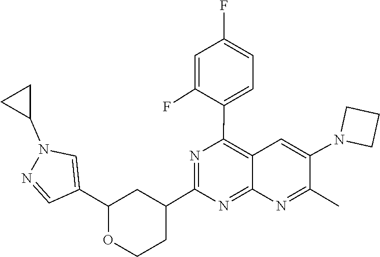 [SMILES: Cc1nc2nc(C3CCOC(c4cnn(C5CC5)c4)C3)nc(-c3ccc(F)cc3F)c2cc1N1CCC1] |
| I-626 | 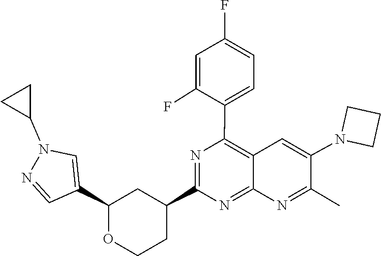 [SMILES: Cc1nc2nc([C@H]3CCO[C@@H](c4cnn(C5CC5)c4)C3)nc(-c3ccc(F)cc3F)c2cc1N1CCC1] |
| I-627 | 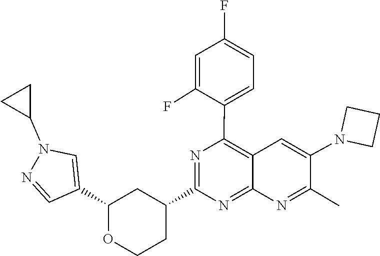 [SMILES: Cc1nc2nc([C@@H]3CCO[C@H](c4cnn(C5CC5)c4)C3)nc(-c3ccc(F)cc3F)c2cc1N1CCC1] |
| I-628 | 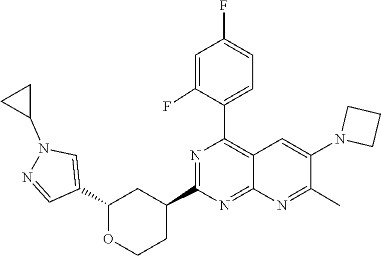 [SMILES: Cc1nc2nc([C@H]3CCO[C@H](c4cnn(C5CC5)c4)C3)nc(-c3ccc(F)cc3F)c2cc1N1CCC1] |
| I-629 | 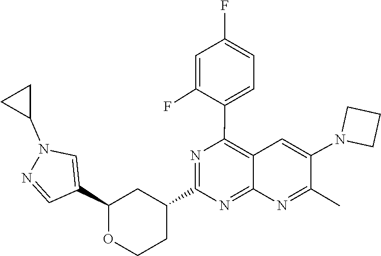 [SMILES: Cc1nc2nc([C@@H]3CCO[C@@H](c4cnn(C5CC5)c4)C3)nc(-c3ccc(F)cc3F)c2cc1N1CCC1] |
| I-630 | 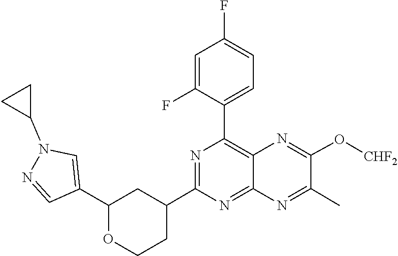 [SMILES: Cc1nc2nc(C3CCOC(c4cnn(C5CC5)c4)C3)nc(-c3ccc(F)cc3F)c2nc1OC(F)F] |
| I-631 | 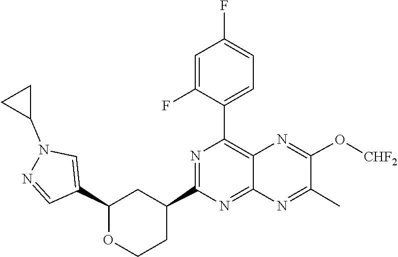 [SMILES: Cc1nc2nc([C@H]3CCO[C@@H](c4cnn(C5CC5)c4)C3)nc(-c3ccc(F)cc3F)c2nc1OC(F)F] |
| I-632 | 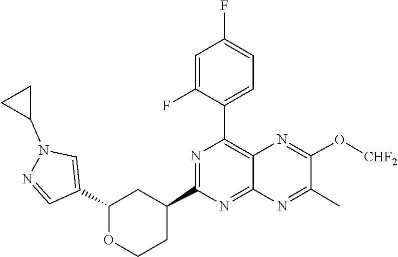 [SMILES: Cc1nc2nc([C@H]3CCO[C@H](c4cnn(C5CC5)c4)C3)nc(-c3ccc(F)cc3F)c2nc1OC(F)F] |
| I-633 | 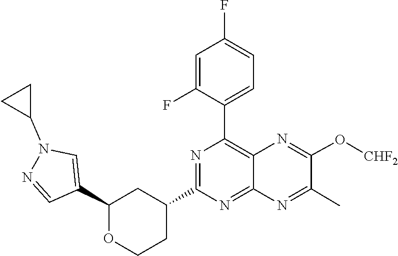 [SMILES: Cc1nc2nc([C@@H]3CCO[C@@H](c4cnn(C5CC5)c4)C3)nc(-c3ccc(F)cc3F)c2nc1OC(F)F] |
| I-634 | 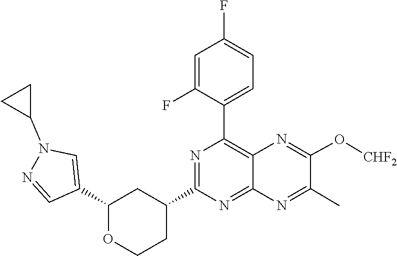 [SMILES: Cc1nc2nc([C@@H]3CCO[C@H](c4cnn(C5CC5)c4)C3)nc(-c3ccc(F)cc3F)c2nc1OC(F)F] |
| I-635 | 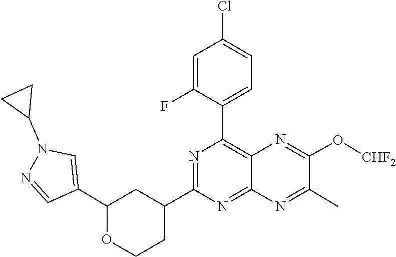 [SMILES: Cc1nc2nc(C3CCOC(c4cnn(C5CC5)c4)C3)nc(-c3ccc(Cl)cc3F)c2nc1OC(F)F] |
| I-636 | 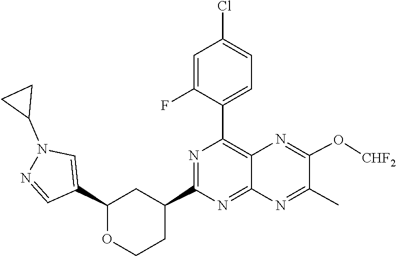 [SMILES: Cc1nc2nc([C@H]3CCO[C@@H](c4cnn(C5CC5)c4)C3)nc(-c3ccc(Cl)cc3F)c2nc1OC(F)F] |
| I-637 | 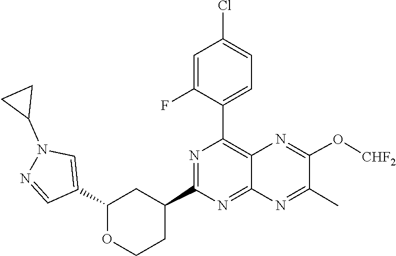 [SMILES: Cc1nc2nc([C@H]3CCO[C@H](c4cnn(C5CC5)c4)C3)nc(-c3ccc(Cl)cc3F)c2nc1OC(F)F] |
| I-638 | 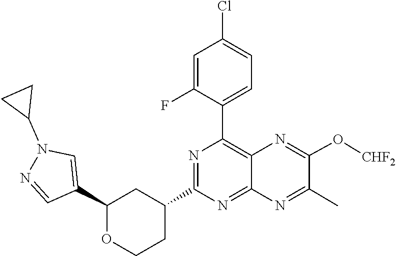 [SMILES: Cc1nc2nc([C@@H]3CCO[C@@H](c4cnn(C5CC5)c4)C3)nc(-c3ccc(Cl)cc3F)c2nc1OC(F)F] |
| I-639 | 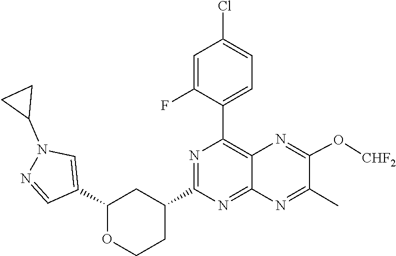 [SMILES: Cc1nc2nc([C@@H]3CCO[C@H](c4cnn(C5CC5)c4)C3)nc(-c3ccc(Cl)cc3F)c2nc1OC(F)F] |
| I-640 | 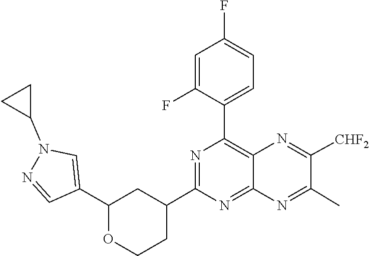 [SMILES: Cc1nc2nc(C3CCOC(c4cnn(C5CC5)c4)C3)nc(-c3ccc(F)cc3F)c2nc1C(F)F] |
| I-641 | 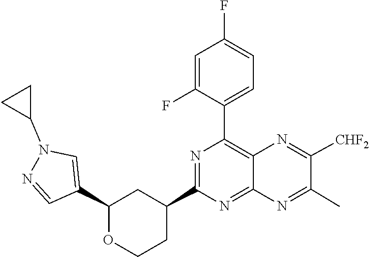 [SMILES: Cc1nc2nc([C@H]3CCO[C@@H](c4cnn(C5CC5)c4)C3)nc(-c3ccc(F)cc3F)c2nc1C(F)F] |
| I-642 | 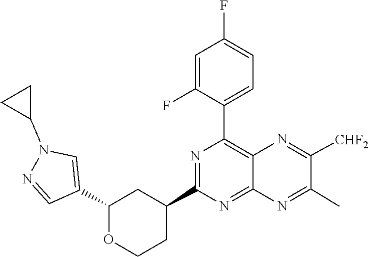 [SMILES: Cc1nc2nc([C@H]3CCO[C@H](c4cnn(C5CC5)c4)C3)nc(-c3ccc(F)cc3F)c2nc1C(F)F] |
| I-643 | 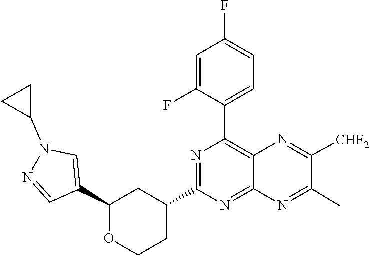 [SMILES: Cc1nc2nc([C@@H]3CCO[C@@H](c4cnn(C5CC5)c4)C3)nc(-c3ccc(F)cc3F)c2nc1C(F)F] |
| I-644 | 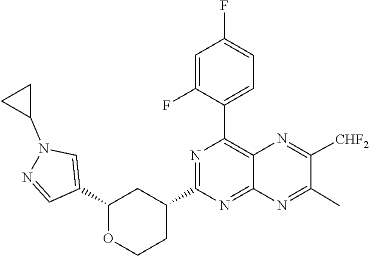 [SMILES: Cc1nc2nc([C@@H]3CCO[C@H](c4cnn(C5CC5)c4)C3)nc(-c3ccc(F)cc3F)c2nc1C(F)F] |
| I-645 | 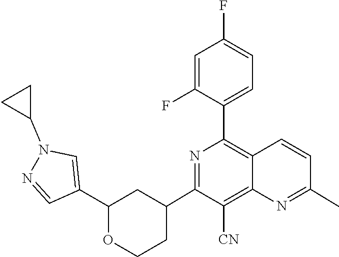 [SMILES: Cc1ccc2c(-c3ccc(F)cc3F)nc(C3CCOC(c4cnn(C5CC5)c4)C3)c(C#N)c2n1] |
| I-646 | 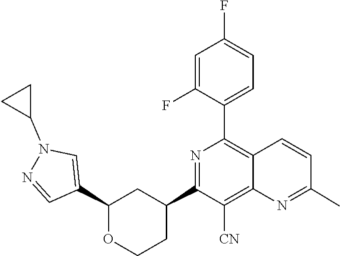 [SMILES: Cc1ccc2c(-c3ccc(F)cc3F)nc([C@H]3CCO[C@@H](c4cnn(C5CC5)c4)C3)c(C#N)c2n1] |
| I-647 | 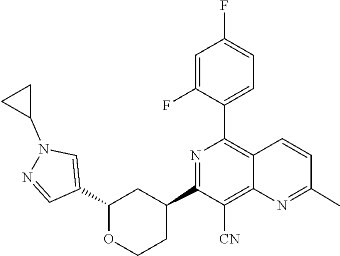 [SMILES: Cc1ccc2c(-c3ccc(F)cc3F)nc([C@H]3CCO[C@H](c4cnn(C5CC5)c4)C3)c(C#N)c2n1] |
| I-648 | 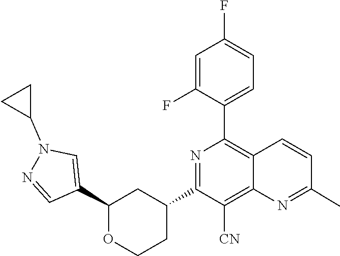 [SMILES: Cc1ccc2c(-c3ccc(F)cc3F)nc([C@@H]3CCO[C@@H](c4cnn(C5CC5)c4)C3)c(C#N)c2n1] |
| I-649 | 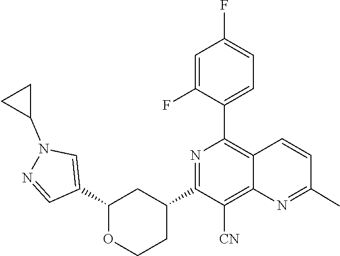 [SMILES: Cc1ccc2c(-c3ccc(F)cc3F)nc([C@@H]3CCO[C@H](c4cnn(C5CC5)c4)C3)c(C#N)c2n1] |
| I-650 | 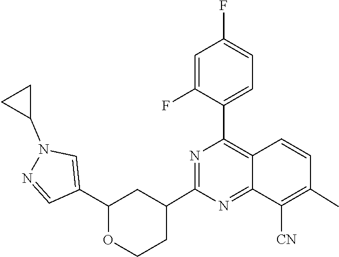 [SMILES: Cc1ccc2c(-c3ccc(F)cc3F)nc(C3CCOC(c4cnn(C5CC5)c4)C3)nc2c1C#N] |
| I-651 | 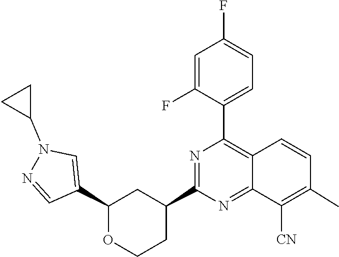 [SMILES: Cc1ccc2c(-c3ccc(F)cc3F)nc([C@H]3CCO[C@@H](c4cnn(C5CC5)c4)C3)nc2c1C#N] |
| I-652 | 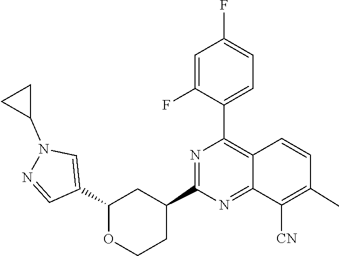 [SMILES: Cc1ccc2c(-c3ccc(F)cc3F)nc([C@H]3CCO[C@H](c4cnn(C5CC5)c4)C3)nc2c1C#N] |
| I-653 | 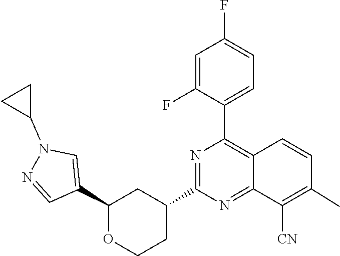 [SMILES: Cc1ccc2c(-c3ccc(F)cc3F)nc([C@@H]3CCO[C@@H](c4cnn(C5CC5)c4)C3)nc2c1C#N] |
| I-654 | 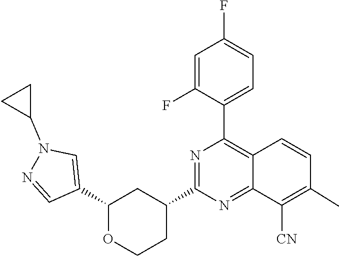 [SMILES: Cc1ccc2c(-c3ccc(F)cc3F)nc([C@@H]3CCO[C@H](c4cnn(C5CC5)c4)C3)nc2c1C#N] |
| I-655 | 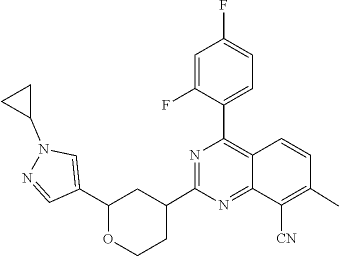 [SMILES: Cc1ccc2c(-c3ccc(F)cc3F)nc(C3CCOC(c4cnn(C5CC5)c4)C3)nc2c1C#N] |
| I-656 | 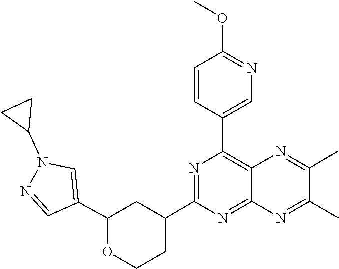 [SMILES: COc1ccc(-c2nc(C3CCOC(c4cnn(C5CC5)c4)C3)nc3nc(C)c(C)nc23)cn1] |
| I-657 | 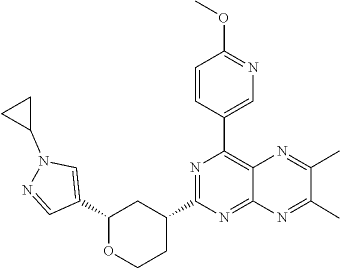 [SMILES: COc1ccc(-c2nc([C@@H]3CCO[C@H](c4cnn(C5CC5)c4)C3)nc3nc(C)c(C)nc23)cn1] |
| I-658 | 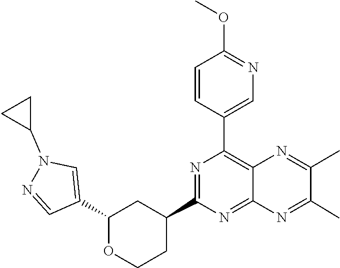 [SMILES: COc1ccc(-c2nc([C@H]3CCO[C@H](c4cnn(C5CC5)c4)C3)nc3nc(C)c(C)nc23)cn1] |
| I-659 | 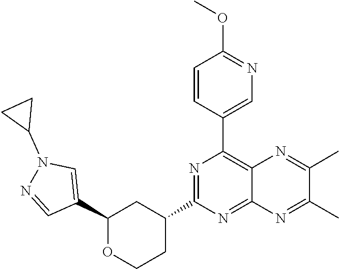 [SMILES: COc1ccc(-c2nc([C@@H]3CCO[C@@H](c4cnn(C5CC5)c4)C3)nc3nc(C)c(C)nc23)cn1] |
| I-660 | 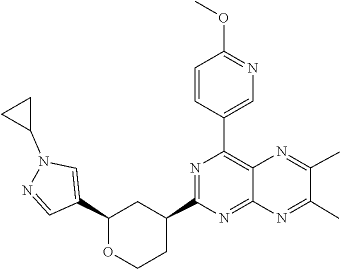 [SMILES: COc1ccc(-c2nc([C@H]3CCO[C@@H](c4cnn(C5CC5)c4)C3)nc3nc(C)c(C)nc23)cn1] |
| I-661 | 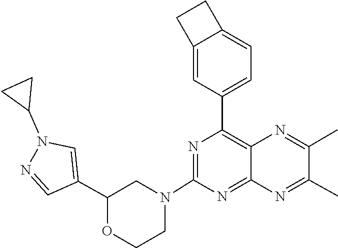 [SMILES: Cc1nc2nc(N3CCOC(c4cnn(C5CC5)c4)C3)nc(-c3ccc4c(c3)CC4)c2nc1C] |
| I-662 | 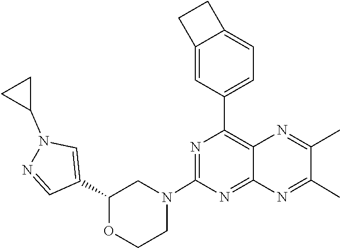 [SMILES: Cc1nc2nc(N3CCO[C@H](c4cnn(C5CC5)c4)C3)nc(-c3ccc4c(c3)CC4)c2nc1C] |
| I-663 | 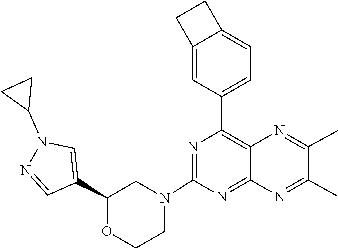 [SMILES: Cc1nc2nc(N3CCO[C@@H](c4cnn(C5CC5)c4)C3)nc(-c3ccc4c(c3)CC4)c2nc1C] |
| I-664 | 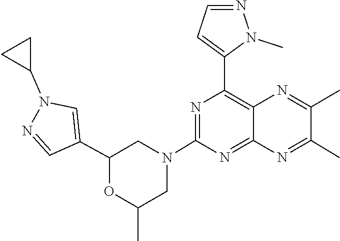 [SMILES: Cc1nc2nc(N3CC(C)OC(c4cnn(C5CC5)c4)C3)nc(-c3ccnn3C)c2nc1C] |
| I-665 | 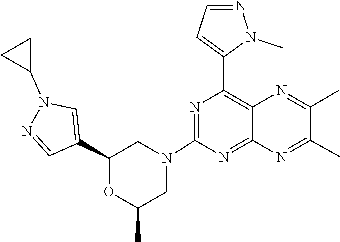 [SMILES: Cc1nc2nc(N3C[C@@H](C)O[C@@H](c4cnn(C5CC5)c4)C3)nc(-c3ccnn3C)c2nc1C] |
| I-666 | 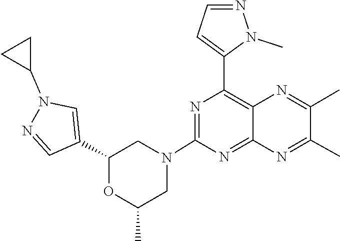 [SMILES: Cc1nc2nc(N3C[C@@H](c4cnn(C5CC5)c4)O[C@@H](C)C3)nc(-c3ccnn3C)c2nc1C] |
| I-667 | 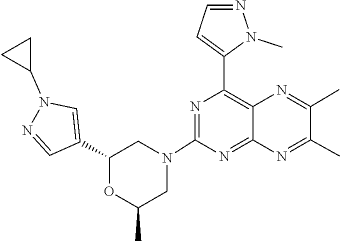 [SMILES: Cc1nc2nc(N3C[C@@H](C)O[C@H](c4cnn(C5CC5)c4)C3)nc(-c3ccnn3C)c2nc1C] |
| I-668 | 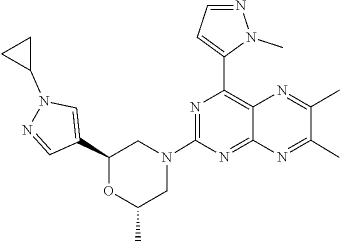 [SMILES: Cc1nc2nc(N3C[C@H](C)O[C@@H](c4cnn(C5CC5)c4)C3)nc(-c3ccnn3C)c2nc1C] |
| I-669 | 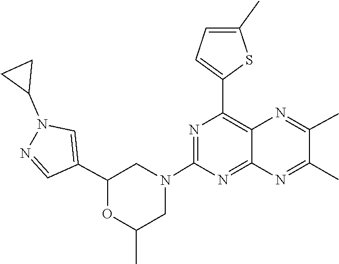 [SMILES: Cc1ccc(-c2nc(N3CC(C)OC(c4cnn(C5CC5)c4)C3)nc3nc(C)c(C)nc23)s1] |
| I-670 | 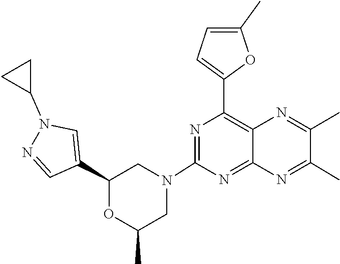 [SMILES: Cc1ccc(-c2nc(N3C[C@@H](C)O[C@@H](c4cnn(C5CC5)c4)C3)nc3nc(C)c(C)nc23)o1] |
| I-671 | 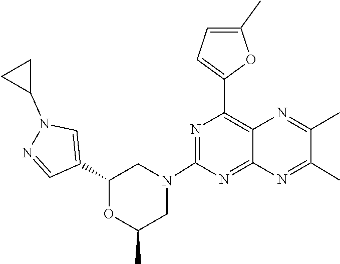 [SMILES: Cc1ccc(-c2nc(N3C[C@@H](C)O[C@H](c4cnn(C5CC5)c4)C3)nc3nc(C)c(C)nc23)o1] |
| I-672 | 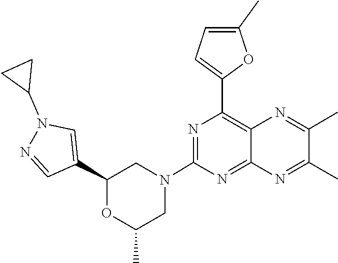 [SMILES: Cc1ccc(-c2nc(N3C[C@H](C)O[C@@H](c4cnn(C5CC5)c4)C3)nc3nc(C)c(C)nc23)o1] |
| I-673 | 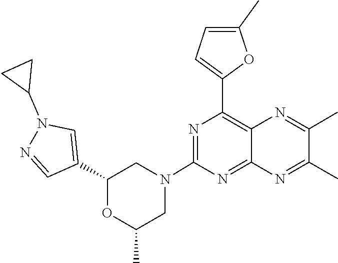 [SMILES: Cc1ccc(-c2nc(N3C[C@@H](c4cnn(C5CC5)c4)O[C@@H](C)C3)nc3nc(C)c(C)nc23)o1] |
| I-674 | 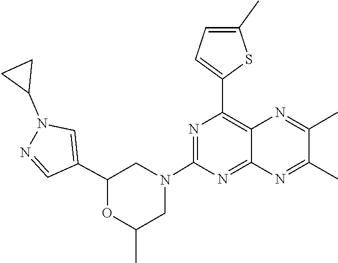 [SMILES: Cc1ccc(-c2nc(N3CC(C)OC(c4cnn(C5CC5)c4)C3)nc3nc(C)c(C)nc23)s1] |
| I-675 | 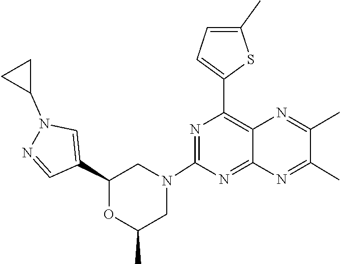 [SMILES: Cc1ccc(-c2nc(N3C[C@@H](C)O[C@@H](c4cnn(C5CC5)c4)C3)nc3nc(C)c(C)nc23)s1] |
| I-676 | 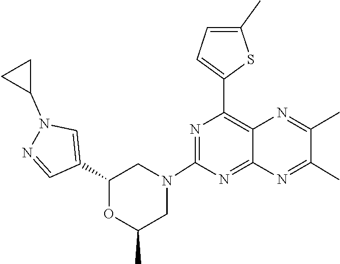 [SMILES: Cc1ccc(-c2nc(N3C[C@@H](C)O[C@H](c4cnn(C5CC5)c4)C3)nc3nc(C)c(C)nc23)s1] |
| I-677 | 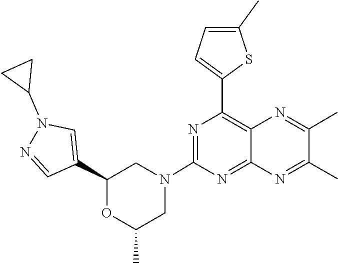 [SMILES: Cc1ccc(-c2nc(N3C[C@H](C)O[C@@H](c4cnn(C5CC5)c4)C3)nc3nc(C)c(C)nc23)s1] |
| I-678 | 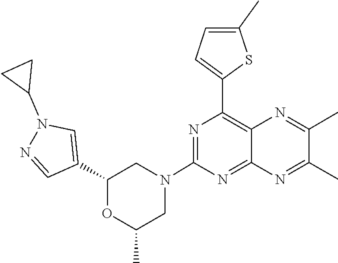 [SMILES: Cc1ccc(-c2nc(N3C[C@@H](c4cnn(C5CC5)c4)O[C@@H](C)C3)nc3nc(C)c(C)nc23)s1] |
| I-679 | 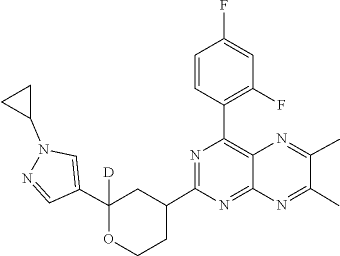 [SMILES: [2H]C1(c2cnn(C3CC3)c2)CC(c2nc(-c3ccc(F)cc3F)c3nc(C)c(C)nc3n2)CCO1] |
| I-680 | 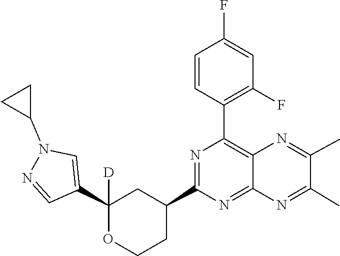 [SMILES: [2H][C@]1(c2cnn(C3CC3)c2)C[C@@H](c2nc(-c3ccc(F)cc3F)c3nc(C)c(C)nc3n2)CCO1] |
| I-681 | 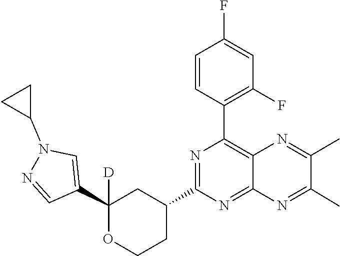 [SMILES: [2H][C@]1(c2cnn(C3CC3)c2)C[C@H](c2nc(-c3ccc(F)cc3F)c3nc(C)c(C)nc3n2)CCO1] |
| I-682 | 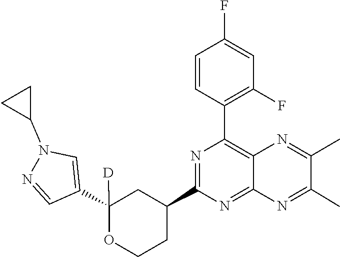 [SMILES: [2H][C@@]1(c2cnn(C3CC3)c2)C[C@@H](c2nc(-c3ccc(F)cc3F)c3nc(C)c(C)nc3n2)CCO1] |
| I-683 | 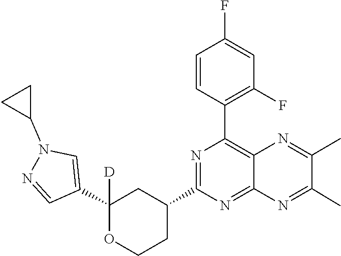 [SMILES: [2H][C@@]1(c2cnn(C3CC3)c2)C[C@H](c2nc(-c3ccc(F)cc3F)c3nc(C)c(C)nc3n2)CCO1] |
| I-684 | 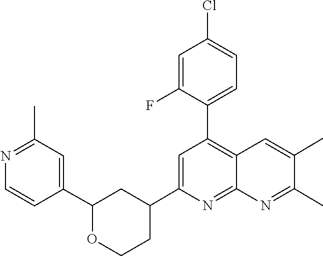 [SMILES: Cc1cc(C2CC(c3cc(-c4ccc(Cl)cc4F)c4cc(C)c(C)nc4n3)CCO2)ccn1] |
| I-685 | 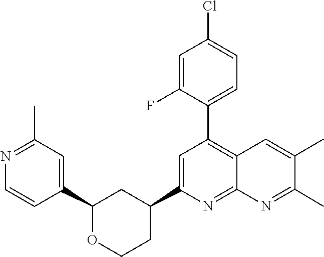 [SMILES: Cc1cc([C@H]2C[C@@H](c3cc(-c4ccc(Cl)cc4F)c4cc(C)c(C)nc4n3)CCO2)ccn1] |
| I-686 | 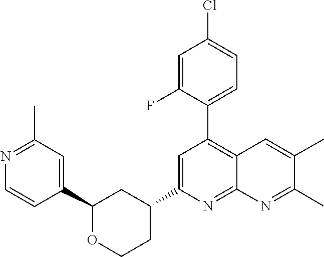 [SMILES: Cc1cc([C@H]2C[C@H](c3cc(-c4ccc(Cl)cc4F)c4cc(C)c(C)nc4n3)CCO2)ccn1] |
| I-687 | 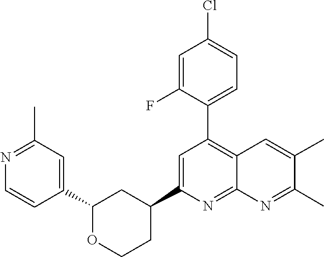 [SMILES: Cc1cc([C@@H]2C[C@@H](c3cc(-c4ccc(Cl)cc4F)c4cc(C)c(C)nc4n3)CCO2)ccn1] |
| I-688 | 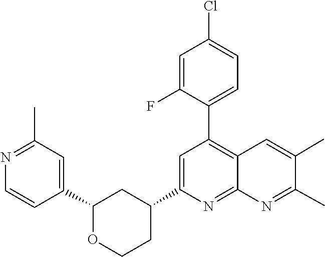 [SMILES: Cc1cc([C@@H]2C[C@H](c3cc(-c4ccc(Cl)cc4F)c4cc(C)c(C)nc4n3)CCO2)ccn1] |
| I-689 | 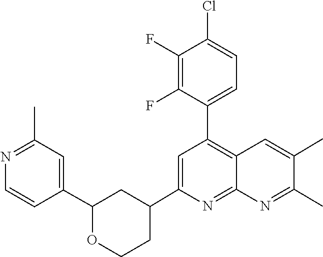 [SMILES: Cc1cc(C2CC(c3cc(-c4ccc(Cl)c(F)c4F)c4cc(C)c(C)nc4n3)CCO2)ccn1] |
| I-690 | 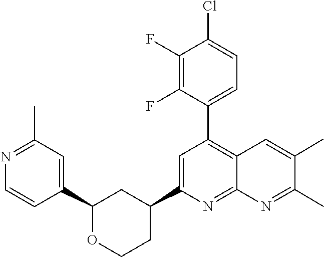 [SMILES: Cc1cc([C@H]2C[C@@H](c3cc(-c4ccc(Cl)c(F)c4F)c4cc(C)c(C)nc4n3)CCO2)ccn1] |
| I-691 | 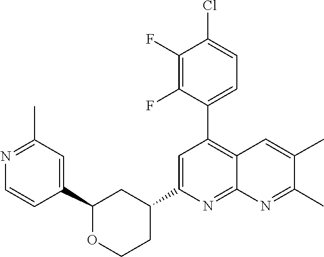 [SMILES: Cc1cc([C@H]2C[C@H](c3cc(-c4ccc(Cl)c(F)c4F)c4cc(C)c(C)nc4n3)CCO2)ccn1] |
| I-692 | 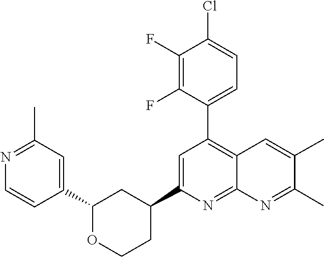 [SMILES: Cc1cc([C@@H]2C[C@@H](c3cc(-c4ccc(Cl)c(F)c4F)c4cc(C)c(C)nc4n3)CCO2)ccn1] |
| I-693 | 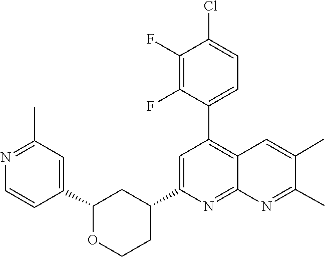 [SMILES: Cc1cc([C@@H]2C[C@H](c3cc(-c4ccc(Cl)c(F)c4F)c4cc(C)c(C)nc4n3)CCO2)ccn1] |
| I-694 | 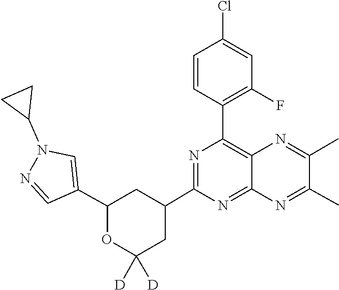 [SMILES: [2H]C1([2H])CC(c2nc(-c3ccc(Cl)cc3F)c3nc(C)c(C)nc3n2)CC(c2cnn(C3CC3)c2)O1] |
| I-695 | 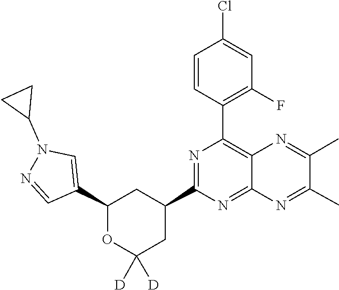 [SMILES: [2H]C1([2H])C[C@H](c2nc(-c3ccc(Cl)cc3F)c3nc(C)c(C)nc3n2)C[C@H](c2cnn(C3CC3)c2)O1] |
| I-696 | 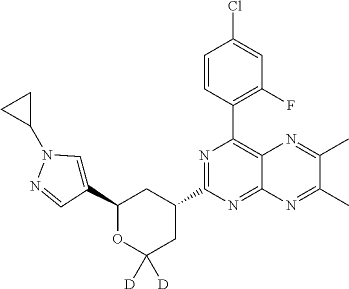 [SMILES: [2H]C1([2H])C[C@@H](c2nc(-c3ccc(Cl)cc3F)c3nc(C)c(C)nc3n2)C[C@H](c2cnn(C3CC3)c2)O1] |
| I-697 | 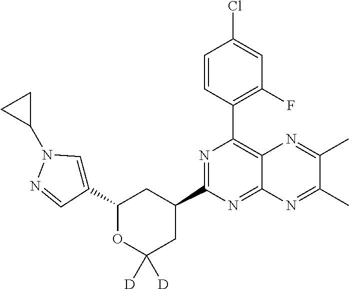 [SMILES: [2H]C1([2H])C[C@H](c2nc(-c3ccc(Cl)cc3F)c3nc(C)c(C)nc3n2)C[C@@H](c2cnn(C3CC3)c2)O1] |
| I-698 | 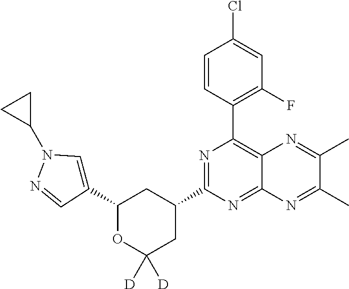 [SMILES: [2H]C1([2H])C[C@@H](c2nc(-c3ccc(Cl)cc3F)c3nc(C)c(C)nc3n2)C[C@@H](c2cnn(C3CC3)c2)O1] |
| I-699 | 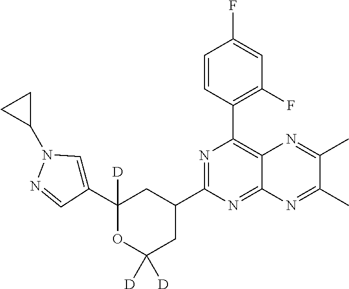 [SMILES: [2H]C1([2H])CC(c2nc(-c3ccc(F)cc3F)c3nc(C)c(C)nc3n2)CC([2H])(c2cnn(C3CC3)c2)O1] |
| I-700 | 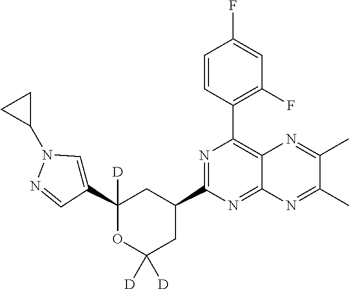 [SMILES: [2H]C1([2H])C[C@H](c2nc(-c3ccc(F)cc3F)c3nc(C)c(C)nc3n2)C[C@]([2H])(c2cnn(C3CC3)c2)O1] |
| I-701 | 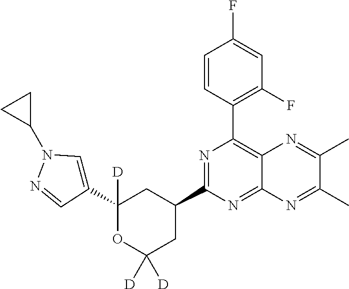 [SMILES: [2H]C1([2H])C[C@H](c2nc(-c3ccc(F)cc3F)c3nc(C)c(C)nc3n2)C[C@@]([2H])(c2cnn(C3CC3)c2)O1] |
| I-702 | 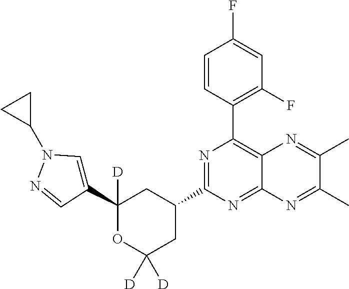 [SMILES: [2H]C1([2H])C[C@@H](c2nc(-c3ccc(F)cc3F)c3nc(C)c(C)nc3n2)C[C@]([2H])(c2cnn(C3CC3)c2)O1] |
| I-703 | 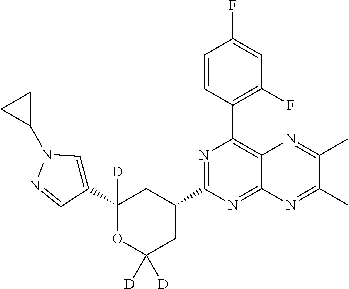 [SMILES: [2H]C1([2H])C[C@@H](c2nc(-c3ccc(F)cc3F)c3nc(C)c(C)nc3n2)C[C@@]([2H])(c2cnn(C3CC3)c2)O1] |
| I-704 | 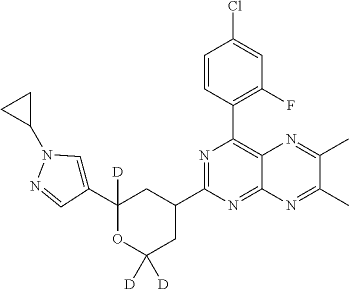 [SMILES: [2H]C1([2H])CC(c2nc(-c3ccc(Cl)cc3F)c3nc(C)c(C)nc3n2)CC([2H])(c2cnn(C3CC3)c2)O1] |
| I-705 | 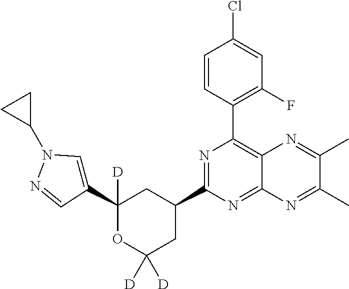 [SMILES: [2H]C1([2H])C[C@H](c2nc(-c3ccc(Cl)cc3F)c3nc(C)c(C)nc3n2)C[C@]([2H])(c2cnn(C3CC3)c2)O1] |
| I-706 | 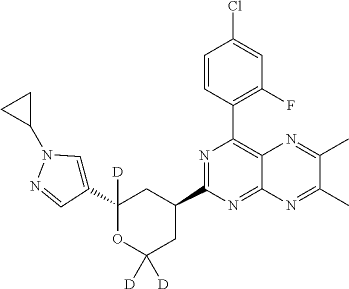 [SMILES: [2H]C1([2H])C[C@H](c2nc(-c3ccc(Cl)cc3F)c3nc(C)c(C)nc3n2)C[C@@]([2H])(c2cnn(C3CC3)c2)O1] |
| I-707 | 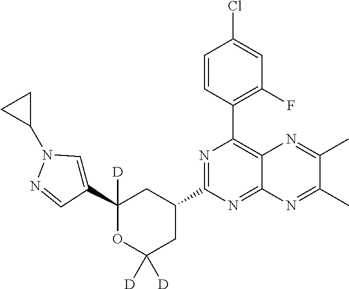 [SMILES: [2H]C1([2H])C[C@@H](c2nc(-c3ccc(Cl)cc3F)c3nc(C)c(C)nc3n2)C[C@]([2H])(c2cnn(C3CC3)c2)O1] |
| I-708 | 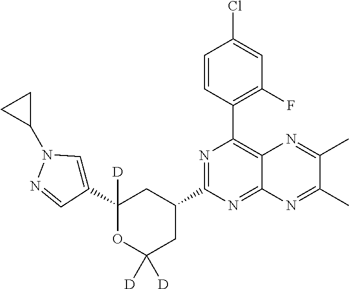 [SMILES: [2H]C1([2H])C[C@@H](c2nc(-c3ccc(Cl)cc3F)c3nc(C)c(C)nc3n2)C[C@@]([2H])(c2cnn(C3CC3)c2)O1] |
| I-709 | 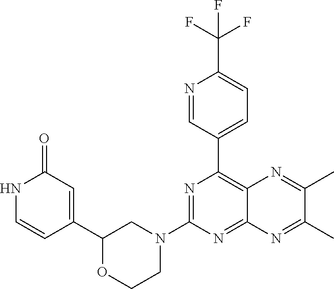 [SMILES: Cc1nc2nc(N3CCOC(c4cc[nH]c(=O)c4)C3)nc(-c3ccc(C(F)(F)F)nc3)c2nc1C] |
| I-710 | 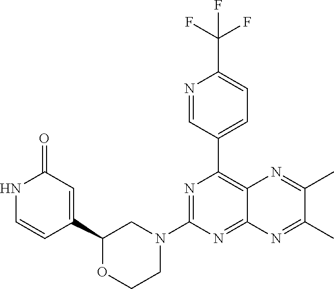 [SMILES: Cc1nc2nc(N3CCO[C@@H](c4cc[nH]c(=O)c4)C3)nc(-c3ccc(C(F)(F)F)nc3)c2nc1C] |
| I-711 | 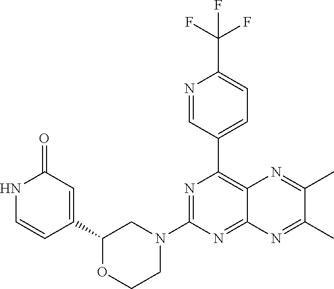 [SMILES: Cc1nc2nc(N3CCO[C@H](c4cc[nH]c(=O)c4)C3)nc(-c3ccc(C(F)(F)F)nc3)c2nc1C] |
| I-712 | 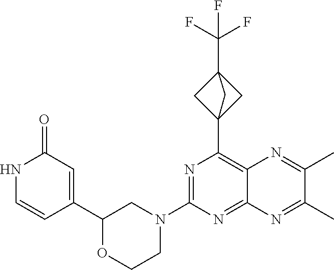 [SMILES: Cc1nc2nc(N3CCOC(c4cc[nH]c(=O)c4)C3)nc(C34CC(C(F)(F)F)(C3)C4)c2nc1C] |
| I-713 | 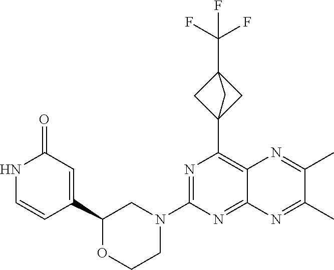 [SMILES: Cc1nc2nc(N3CCO[C@@H](c4cc[nH]c(=O)c4)C3)nc(C34CC(C(F)(F)F)(C3)C4)c2nc1C] |
| I-714 | 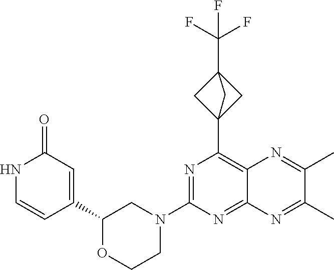 [SMILES: Cc1nc2nc(N3CCO[C@H](c4cc[nH]c(=O)c4)C3)nc(C34CC(C(F)(F)F)(C3)C4)c2nc1C] |
| I-715 | 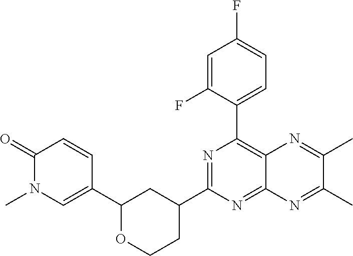 |
| I-716 | 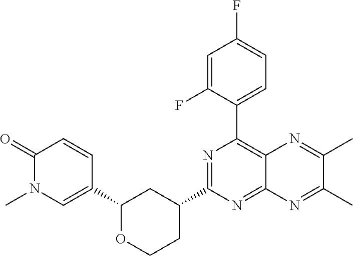 |
| I-717 | 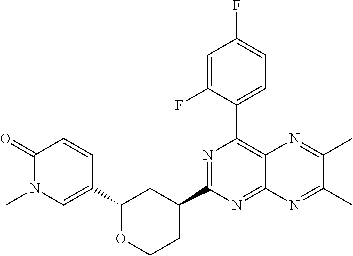 |
| I-718 | 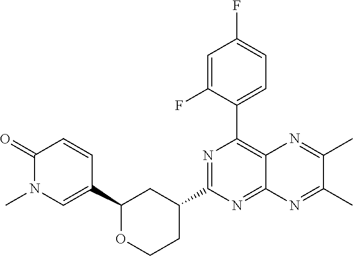 |
| I-719 | 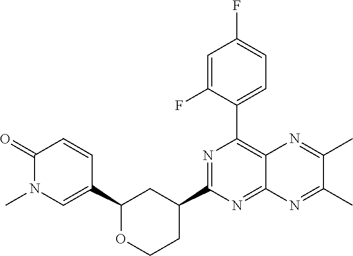 |
| I-720 | 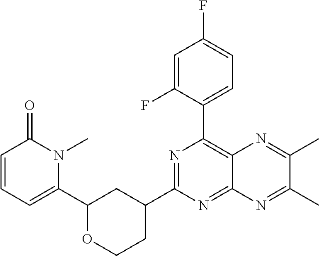 [SMILES: Cc1nc2nc(C3CCOC(c4cccc(=O)n4C)C3)nc(-c3ccc(F)cc3F)c2nc1C] |
| I-721 | 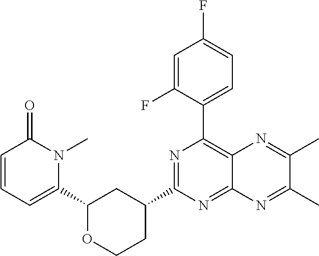 [SMILES: Cc1nc2nc([C@@H]3CCO[C@H](c4cccc(=O)n4C)C3)nc(-c3ccc(F)cc3F)c2nc1C] |
| I-722 | 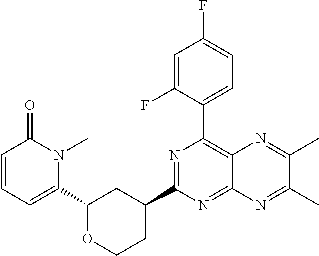 [SMILES: Cc1nc2nc([C@H]3CCO[C@H](c4cccc(=O)n4C)C3)nc(-c3ccc(F)cc3F)c2nc1C] |
| I-723 | 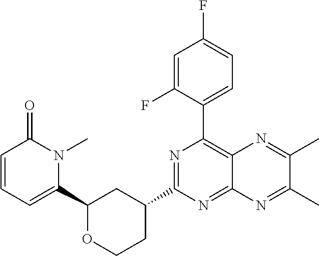 [SMILES: Cc1nc2nc([C@@H]3CCO[C@@H](c4cccc(=O)n4C)C3)nc(-c3ccc(F)cc3F)c2nc1C] |
| I-724 | 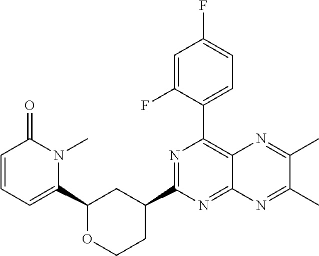 [SMILES: Cc1nc2nc([C@H]3CCO[C@@H](c4cccc(=O)n4C)C3)nc(-c3ccc(F)cc3F)c2nc1C] |
| I-725 | 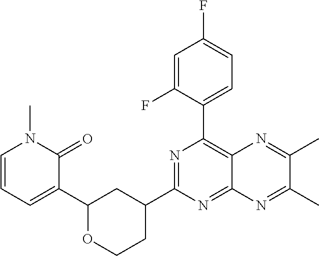 [SMILES: Cc1nc2nc(C3CCOC(c4cccn(C)c4=O)C3)nc(-c3ccc(F)cc3F)c2nc1C] |
| I-726 | 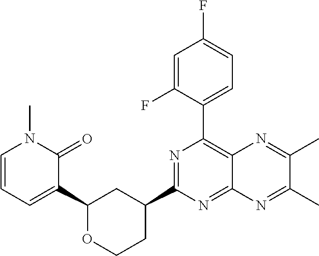 [SMILES: Cc1nc2nc([C@H]3CCO[C@@H](c4cccn(C)c4=O)C3)nc(-c3ccc(F)cc3F)c2nc1C] |
| I-727 | 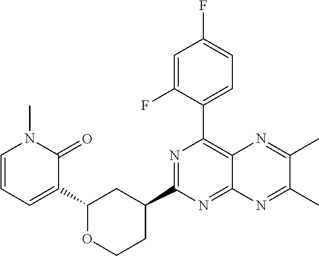 [SMILES: Cc1nc2nc([C@H]3CCO[C@H](c4cccn(C)c4=O)C3)nc(-c3ccc(F)cc3F)c2nc1C] |
| I-728 | 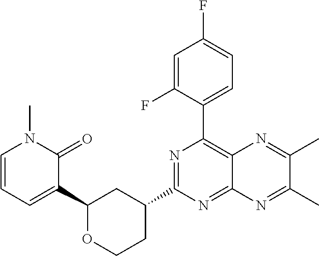 [SMILES: Cc1nc2nc([C@@H]3CCO[C@@H](c4cccn(C)c4=O)C3)nc(-c3ccc(F)cc3F)c2nc1C] |
| I-729 | 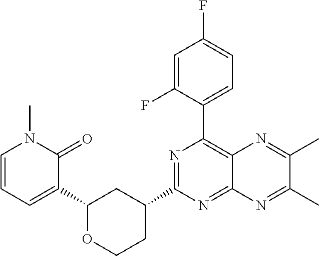 [SMILES: Cc1nc2nc([C@@H]3CCO[C@H](c4cccn(C)c4=O)C3)nc(-c3ccc(F)cc3F)c2nc1C] |
| I-730 | 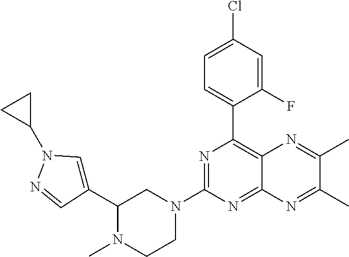 [SMILES: Cc1nc2nc(N3CCN(C)C(c4cnn(C5CC5)c4)C3)nc(-c3ccc(Cl)cc3F)c2nc1C] |
| I-731 | 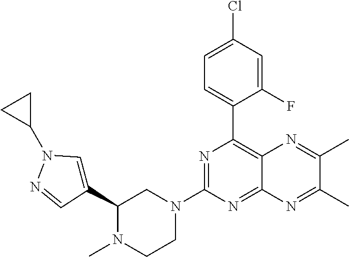 [SMILES: Cc1nc2nc(N3CCN(C)[C@@H](c4cnn(C5CC5)c4)C3)nc(-c3ccc(Cl)cc3F)c2nc1C] |
| I-732 | 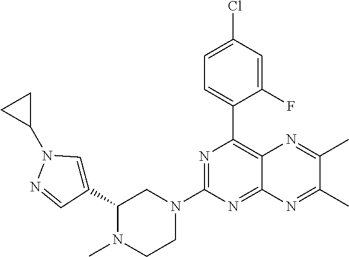 [SMILES: Cc1nc2nc(N3CCN(C)[C@H](c4cnn(C5CC5)c4)C3)nc(-c3ccc(Cl)cc3F)c2nc1C] |
| I-733 | 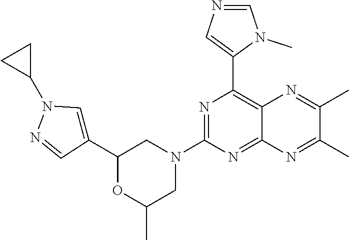 [SMILES: Cc1nc2nc(N3CC(C)OC(c4cnn(C5CC5)c4)C3)nc(-c3cncn3C)c2nc1C] |
| I-734 | 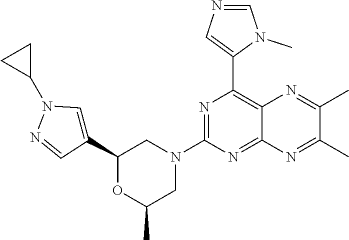 [SMILES: Cc1nc2nc(N3C[C@@H](C)O[C@@H](c4cnn(C5CC5)c4)C3)nc(-c3cncn3C)c2nc1C] |
| I-735 | 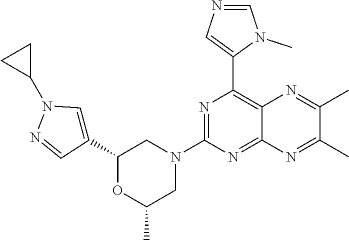 [SMILES: Cc1nc2nc(N3C[C@@H](c4cnn(C5CC5)c4)O[C@@H](C)C3)nc(-c3cncn3C)c2nc1C] |
| I-736 | 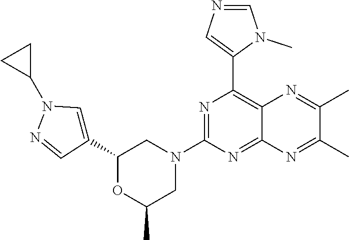 [SMILES: Cc1nc2nc(N3C[C@@H](C)O[C@H](c4cnn(C5CC5)c4)C3)nc(-c3cncn3C)c2nc1C] |
| I-737 | 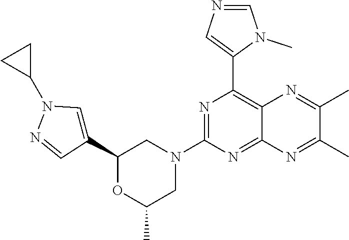 [SMILES: Cc1nc2nc(N3C[C@H](C)O[C@@H](c4cnn(C5CC5)c4)C3)nc(-c3cncn3C)c2nc1C] |
| I-738 | 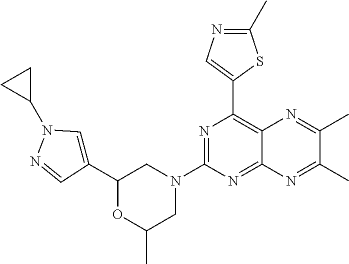 [SMILES: Cc1ncc(-c2nc(N3CC(C)OC(c4cnn(C5CC5)c4)C3)nc3nc(C)c(C)nc23)s1] |
| I-739 | 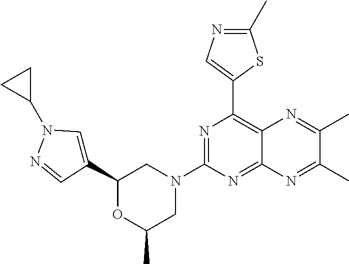 [SMILES: Cc1ncc(-c2nc(N3C[C@@H](C)O[C@@H](c4cnn(C5CC5)c4)C3)nc3nc(C)c(C)nc23)s1] |
| I-740 | 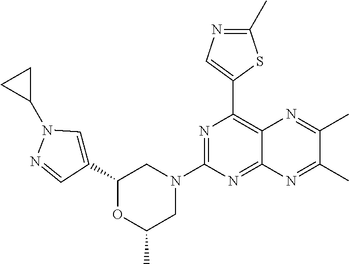 [SMILES: Cc1ncc(-c2nc(N3C[C@@H](c4cnn(C5CC5)c4)O[C@@H](C)C3)nc3nc(C)c(C)nc23)s1] |
| I-741 | 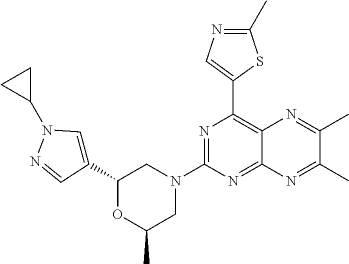 [SMILES: Cc1ncc(-c2nc(N3C[C@@H](C)O[C@H](c4cnn(C5CC5)c4)C3)nc3nc(C)c(C)nc23)s1] |
| I-742 | 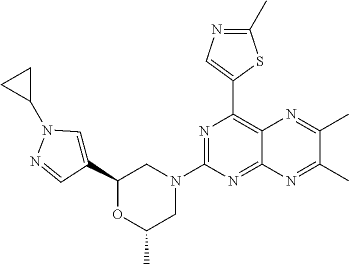 [SMILES: Cc1ncc(-c2nc(N3C[C@H](C)O[C@@H](c4cnn(C5CC5)c4)C3)nc3nc(C)c(C)nc23)s1] |
| I-743 | 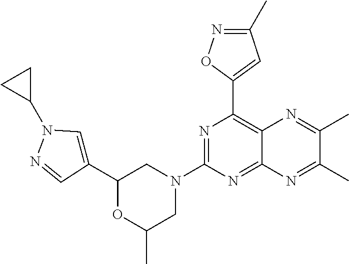 [SMILES: Cc1cc(-c2nc(N3CC(C)OC(c4cnn(C5CC5)c4)C3)nc3nc(C)c(C)nc23)on1] |
| I-744 | 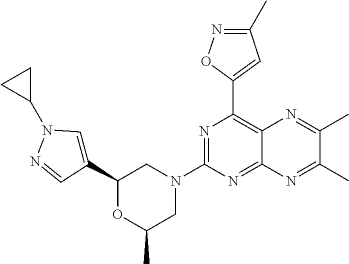 [SMILES: Cc1cc(-c2nc(N3C[C@@H](C)O[C@@H](c4cnn(C5CC5)c4)C3)nc3nc(C)c(C)nc23)on1] |
| I-745 | 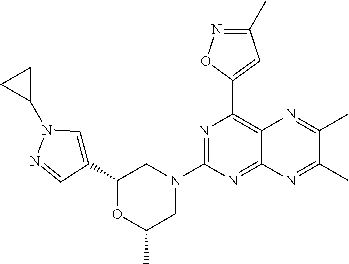 [SMILES: Cc1cc(-c2nc(N3C[C@@H](c4cnn(C5CC5)c4)O[C@@H](C)C3)nc3nc(C)c(C)nc23)on1] |
| I-746 | 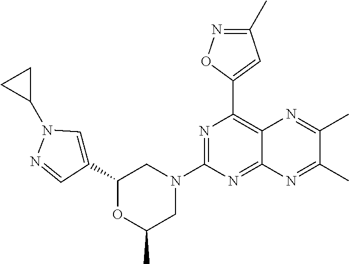 [SMILES: Cc1cc(-c2nc(N3C[C@@H](C)O[C@H](c4cnn(C5CC5)c4)C3)nc3nc(C)c(C)nc23)on1] |
| I-747 | 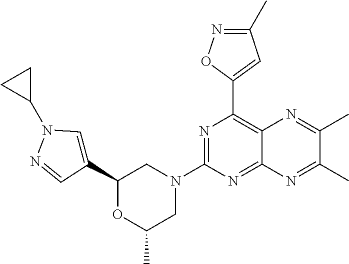 [SMILES: Cc1cc(-c2nc(N3C[C@H](C)O[C@@H](c4cnn(C5CC5)c4)C3)nc3nc(C)c(C)nc23)on1] |
| I-748 | 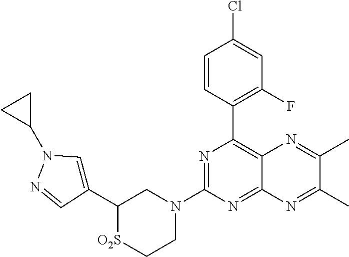 [SMILES: Cc1nc2nc(N3CCS(=O)(=O)C(c4cnn(C5CC5)c4)C3)nc(-c3ccc(Cl)cc3F)c2nc1C] |
| I-749 | 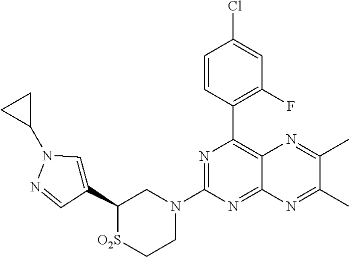 [SMILES: Cc1nc2nc(N3CCS(=O)(=O)[C@@H](c4cnn(C5CC5)c4)C3)nc(-c3ccc(Cl)cc3F)c2nc1C] |
| I-750 | 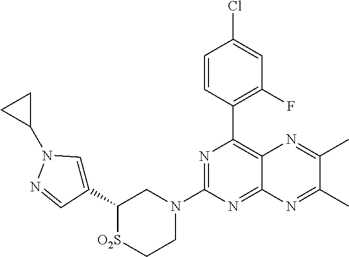 [SMILES: Cc1nc2nc(N3CCS(=O)(=O)[C@H](c4cnn(C5CC5)c4)C3)nc(-c3ccc(Cl)cc3F)c2nc1C] |
| I-751 | 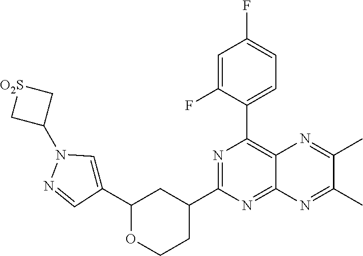 [SMILES: Cc1nc2nc(C3CCOC(c4cnn(C5CS(=O)(=O)C5)c4)C3)nc(-c3ccc(F)cc3F)c2nc1C] |
| I-752 | 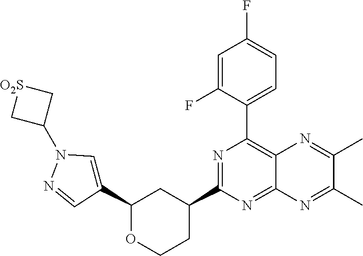 [SMILES: Cc1nc2nc([C@H]3CCO[C@@H](c4cnn(C5CS(=O)(=O)C5)c4)C3)nc(-c3ccc(F)cc3F)c2nc1C] |
| I-753 | 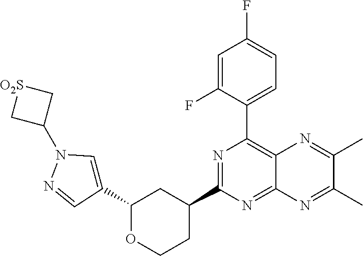 [SMILES: Cc1nc2nc([C@H]3CCO[C@H](c4cnn(C5CS(=O)(=O)C5)c4)C3)nc(-c3ccc(F)cc3F)c2nc1C] |
| I-754 | 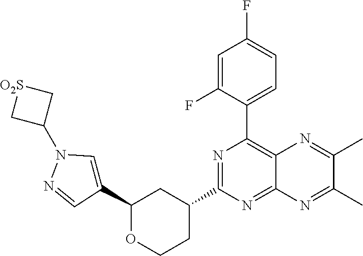 [SMILES: Cc1nc2nc([C@@H]3CCO[C@@H](c4cnn(C5CS(=O)(=O)C5)c4)C3)nc(-c3ccc(F)cc3F)c2nc1C] |
| I-755 | 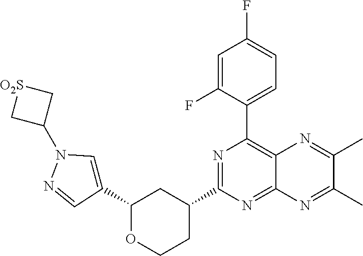 [SMILES: Cc1nc2nc([C@@H]3CCO[C@H](c4cnn(C5CS(=O)(=O)C5)c4)C3)nc(-c3ccc(F)cc3F)c2nc1C] |
| I-756 | 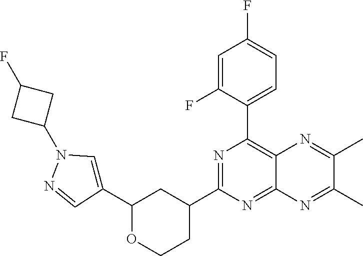 [SMILES: Cc1nc2nc(C3CCOC(c4cnn(C5CC(F)C5)c4)C3)nc(-c3ccc(F)cc3F)c2nc1C] |
| I-757 | 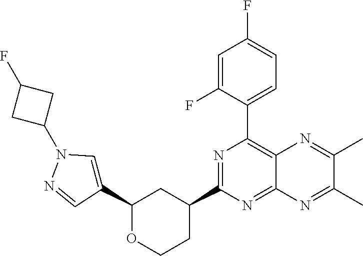 [SMILES: Cc1nc2nc([C@H]3CCO[C@@H](c4cnn(C5CC(F)C5)c4)C3)nc(-c3ccc(F)cc3F)c2nc1C] |
| I-758 | 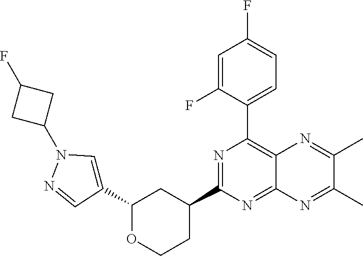 [SMILES: Cc1nc2nc([C@H]3CCO[C@H](c4cnn(C5CC(F)C5)c4)C3)nc(-c3ccc(F)cc3F)c2nc1C] |
| I-759 | 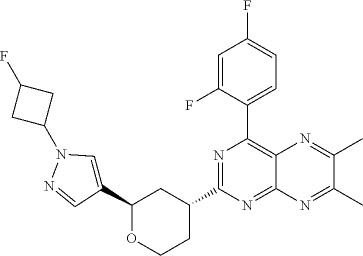 [SMILES: Cc1nc2nc([C@@H]3CCO[C@@H](c4cnn(C5CC(F)C5)c4)C3)nc(-c3ccc(F)cc3F)c2nc1C] |
| I-760 | 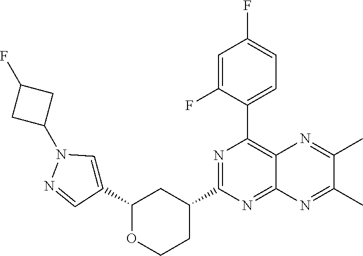 [SMILES: Cc1nc2nc([C@@H]3CCO[C@H](c4cnn(C5CC(F)C5)c4)C3)nc(-c3ccc(F)cc3F)c2nc1C] |
| I-761 | 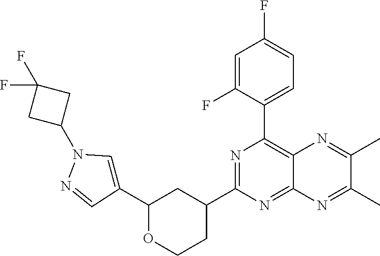 [SMILES: Cc1nc2nc(C3CCOC(c4cnn(C5CC(F)(F)C5)c4)C3)nc(-c3ccc(F)cc3F)c2nc1C] |
| I-762 | 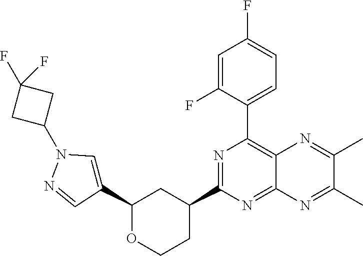 [SMILES: Cc1nc2nc([C@H]3CCO[C@@H](c4cnn(C5CC(F)(F)C5)c4)C3)nc(-c3ccc(F)cc3F)c2nc1C] |
| I-763 | 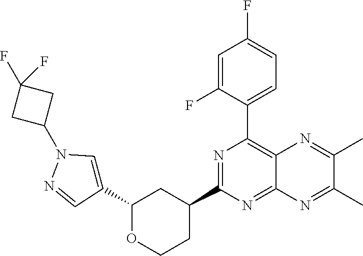 [SMILES: Cc1nc2nc([C@H]3CCO[C@H](c4cnn(C5CC(F)(F)C5)c4)C3)nc(-c3ccc(F)cc3F)c2nc1C] |
| I-764 | 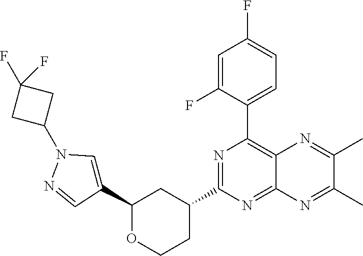 [SMILES: Cc1nc2nc([C@@H]3CCO[C@@H](c4cnn(C5CC(F)(F)C5)c4)C3)nc(-c3ccc(F)cc3F)c2nc1C] |
| I-765 | 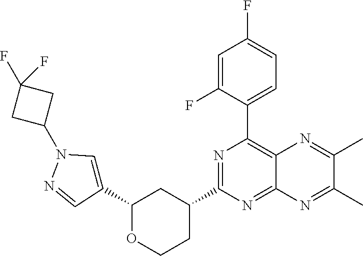 [SMILES: Cc1nc2nc([C@@H]3CCO[C@H](c4cnn(C5CC(F)(F)C5)c4)C3)nc(-c3ccc(F)cc3F)c2nc1C] |
| I-766 | 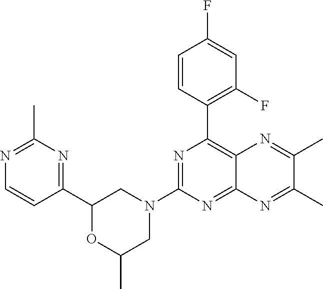 [SMILES: Cc1nccc(C2CN(c3nc(-c4ccc(F)cc4F)c4nc(C)c(C)nc4n3)CC(C)O2)n1] |
| I-767 | 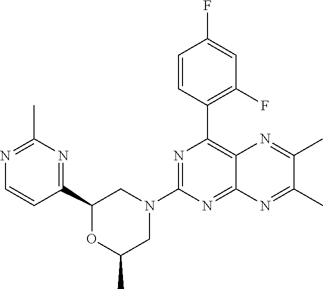 [SMILES: Cc1nccc([C@H]2CN(c3nc(-c4ccc(F)cc4F)c4nc(C)c(C)nc4n3)C[C@@H](C)O2)n1] |
| I-768 | 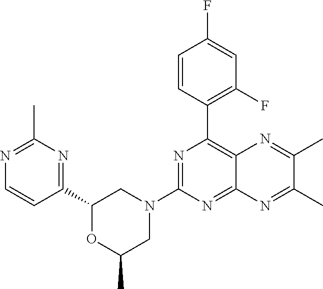 [SMILES: Cc1nccc([C@@H]2CN(c3nc(-c4ccc(F)cc4F)c4nc(C)c(C)nc4n3)C[C@@H](C)O2)n1] |
| I-769 | 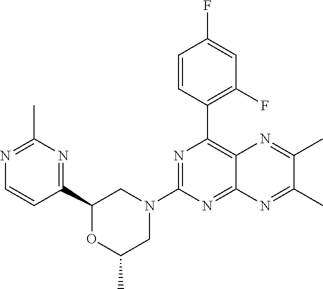 [SMILES: Cc1nccc([C@H]2CN(c3nc(-c4ccc(F)cc4F)c4nc(C)c(C)nc4n3)C[C@H](C)O2)n1] |
| I-770 | 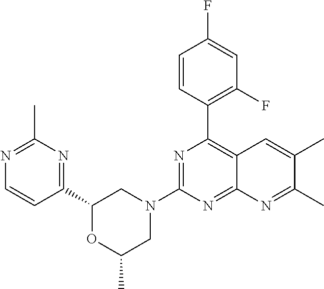 [SMILES: Cc1nccc([C@@H]2CN(c3nc(-c4ccc(F)cc4F)c4cc(C)c(C)nc4n3)C[C@H](C)O2)n1] |
| I-771 | 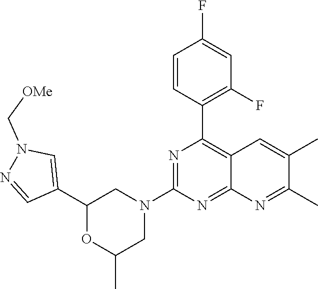 [SMILES: COCn1cc(C2CN(c3nc(-c4ccc(F)cc4F)c4cc(C)c(C)nc4n3)CC(C)O2)cn1] |
| I-772 | 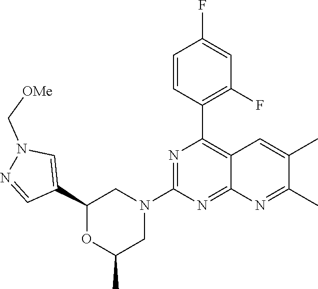 [SMILES: COCn1cc([C@H]2CN(c3nc(-c4ccc(F)cc4F)c4cc(C)c(C)nc4n3)C[C@@H](C)O2)cn1] |
| I-773 | 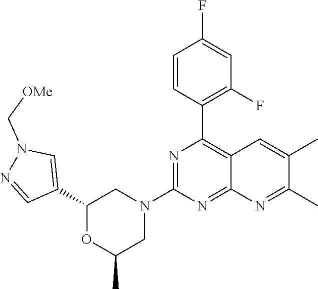 [SMILES: COCn1cc([C@@H]2CN(c3nc(-c4ccc(F)cc4F)c4cc(C)c(C)nc4n3)C[C@@H](C)O2)cn1] |
| I-774 | 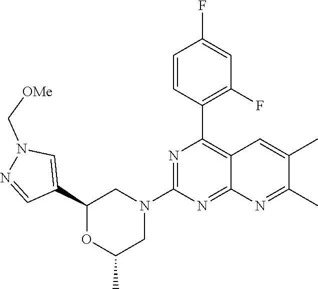 [SMILES: COCn1cc([C@H]2CN(c3nc(-c4ccc(F)cc4F)c4cc(C)c(C)nc4n3)C[C@H](C)O2)cn1] |
| I-775 | 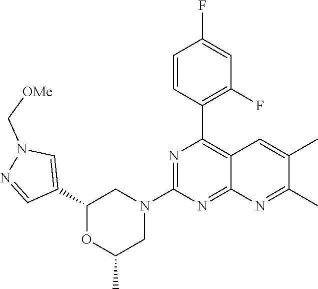 [SMILES: COCn1cc([C@@H]2CN(c3nc(-c4ccc(F)cc4F)c4cc(C)c(C)nc4n3)C[C@H](C)O2)cn1] |
| I-776 | 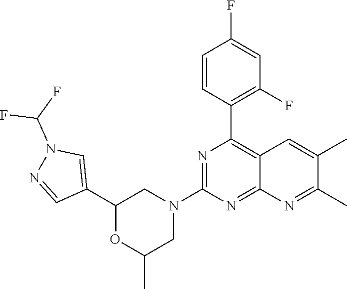 [SMILES: Cc1cc2c(-c3ccc(F)cc3F)nc(N3CC(C)OC(c4cnn(C(F)F)c4)C3)nc2nc1C] |
| I-777 | 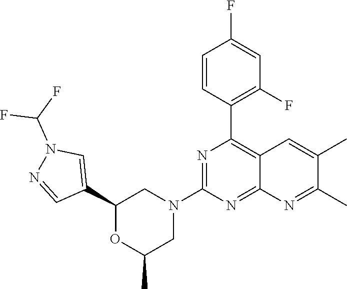 [SMILES: Cc1cc2c(-c3ccc(F)cc3F)nc(N3C[C@@H](C)O[C@@H](c4cnn(C(F)F)c4)C3)nc2nc1C] |
| I-778 | 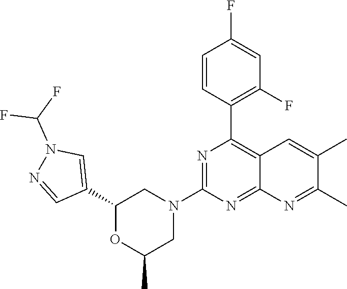 [SMILES: Cc1cc2c(-c3ccc(F)cc3F)nc(N3C[C@@H](C)O[C@H](c4cnn(C(F)F)c4)C3)nc2nc1C] |
| I-779 | 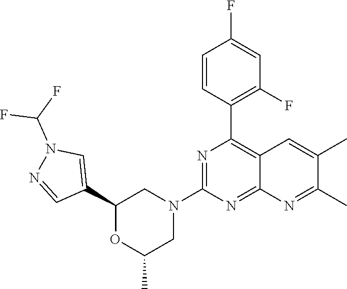 [SMILES: Cc1cc2c(-c3ccc(F)cc3F)nc(N3C[C@H](C)O[C@@H](c4cnn(C(F)F)c4)C3)nc2nc1C] |
| I-780 | 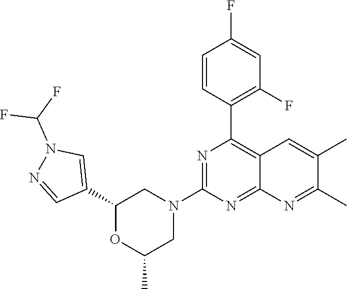 [SMILES: Cc1cc2c(-c3ccc(F)cc3F)nc(N3C[C@@H](c4cnn(C(F)F)c4)O[C@@H](C)C3)nc2nc1C] |
| I-781 | 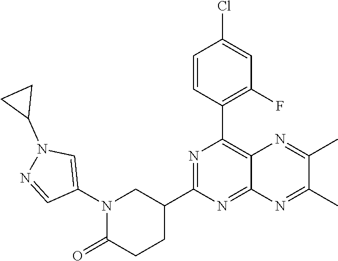 [SMILES: Cc1nc2nc(C3CCC(=O)N(c4cnn(C5CC5)c4)C3)nc(-c3ccc(Cl)cc3F)c2nc1C] |
| I-782 | 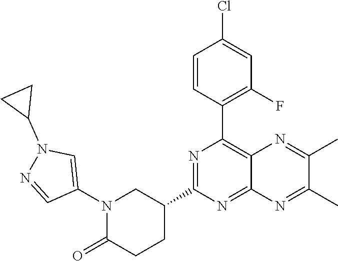 [SMILES: Cc1nc2nc([C@@H]3CCC(=O)N(c4cnn(C5CC5)c4)C3)nc(-c3ccc(Cl)cc3F)c2nc1C] |
| I-783 | 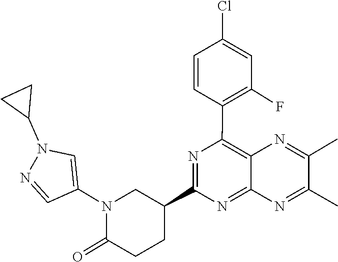 [SMILES: Cc1nc2nc([C@H]3CCC(=O)N(c4cnn(C5CC5)c4)C3)nc(-c3ccc(Cl)cc3F)c2nc1C] |
| I-784 | 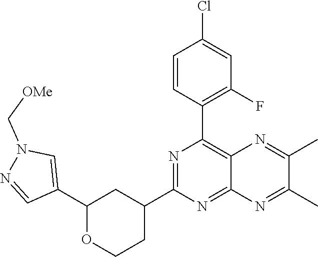 [SMILES: COCn1cc(C2CC(c3nc(-c4ccc(Cl)cc4F)c4nc(C)c(C)nc4n3)CCO2)cn1] |
| I-785 | 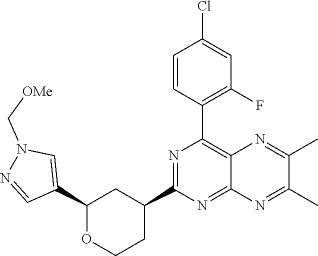 [SMILES: COCn1cc([C@H]2C[C@@H](c3nc(-c4ccc(Cl)cc4F)c4nc(C)c(C)nc4n3)CCO2)cn1] |
| I-786 | 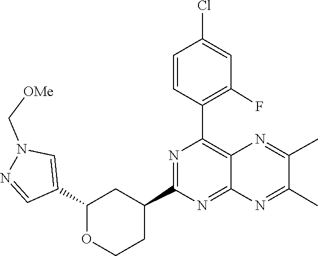 [SMILES: COCn1cc([C@@H]2C[C@@H](c3nc(-c4ccc(Cl)cc4F)c4nc(C)c(C)nc4n3)CCO2)cn1] |
| I-787 | 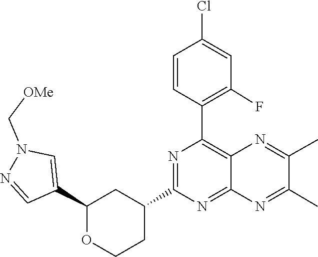 [SMILES: COCn1cc([C@H]2C[C@H](c3nc(-c4ccc(Cl)cc4F)c4nc(C)c(C)nc4n3)CCO2)cn1] |
| I-788 | 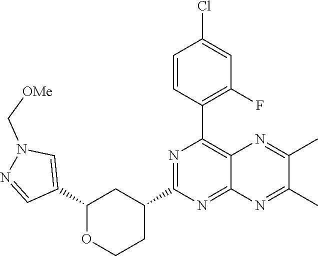 [SMILES: COCn1cc([C@@H]2C[C@H](c3nc(-c4ccc(Cl)cc4F)c4nc(C)c(C)nc4n3)CCO2)cn1] |
| I-789 | 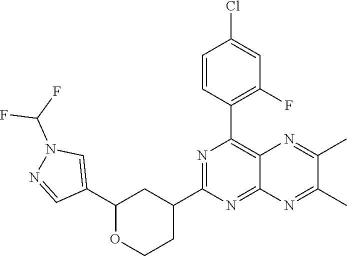 [SMILES: Cc1nc2nc(C3CCOC(c4cnn(C(F)F)c4)C3)nc(-c3ccc(Cl)cc3F)c2nc1C] |
| I-790 | 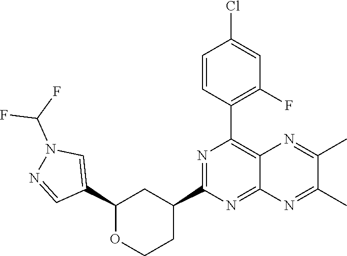 [SMILES: Cc1nc2nc([C@H]3CCO[C@@H](c4cnn(C(F)F)c4)C3)nc(-c3ccc(Cl)cc3F)c2nc1C] |
| I-791 | 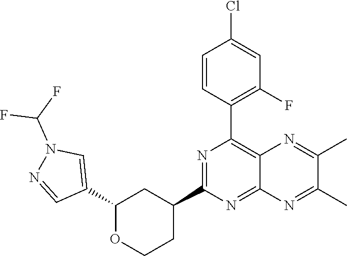 [SMILES: Cc1nc2nc([C@H]3CCO[C@H](c4cnn(C(F)F)c4)C3)nc(-c3ccc(Cl)cc3F)c2nc1C] |
| I-792 | 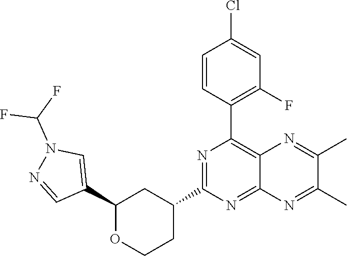 [SMILES: Cc1nc2nc([C@@H]3CCO[C@@H](c4cnn(C(F)F)c4)C3)nc(-c3ccc(Cl)cc3F)c2nc1C] |
| I-793 | 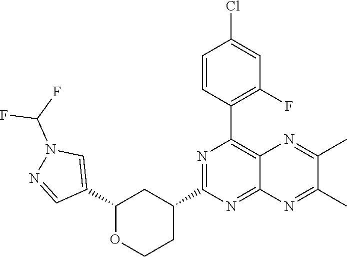 [SMILES: Cc1nc2nc([C@@H]3CCO[C@H](c4cnn(C(F)F)c4)C3)nc(-c3ccc(Cl)cc3F)c2nc1C] |
| I-794 | 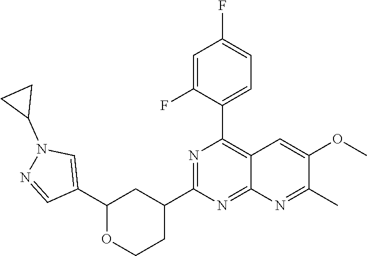 [SMILES: COc1cc2c(-c3ccc(F)cc3F)nc(C3CCOC(c4cnn(C5CC5)c4)C3)nc2nc1C] |
| I-795 | 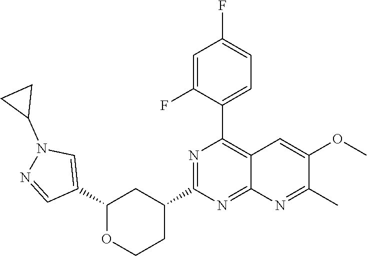 [SMILES: COc1cc2c(-c3ccc(F)cc3F)nc([C@@H]3CCO[C@H](c4cnn(C5CC5)c4)C3)nc2nc1C] |
| I-796 | 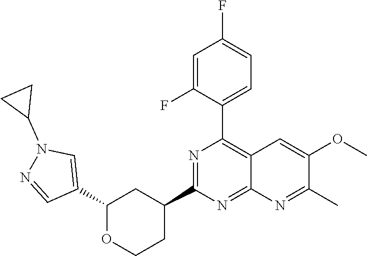 [SMILES: COc1cc2c(-c3ccc(F)cc3F)nc([C@H]3CCO[C@H](c4cnn(C5CC5)c4)C3)nc2nc1C] |
| I-797 | 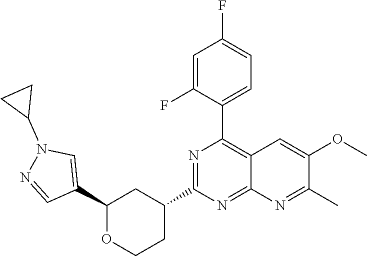 [SMILES: COc1cc2c(-c3ccc(F)cc3F)nc([C@@H]3CCO[C@@H](c4cnn(C5CC5)c4)C3)nc2nc1C] |
| I-798 | 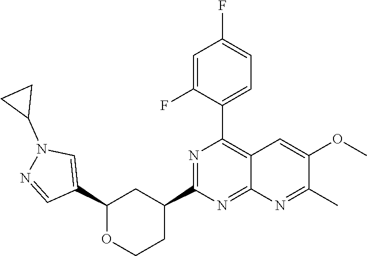 [SMILES: COc1cc2c(-c3ccc(F)cc3F)nc([C@H]3CCO[C@@H](c4cnn(C5CC5)c4)C3)nc2nc1C] |
| I-799 | 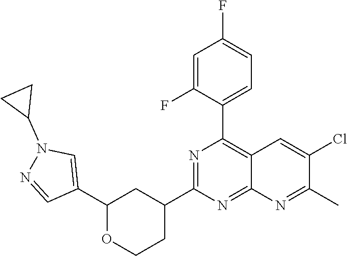 [SMILES: Cc1nc2nc(C3CCOC(c4cnn(C5CC5)c4)C3)nc(-c3ccc(F)cc3F)c2cc1Cl] |
| I-800 | 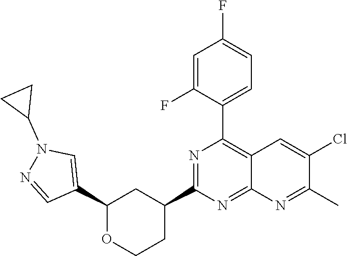 [SMILES: Cc1nc2nc([C@H]3CCO[C@@H](c4cnn(C5CC5)c4)C3)nc(-c3ccc(F)cc3F)c2cc1Cl] |
| I-801 | 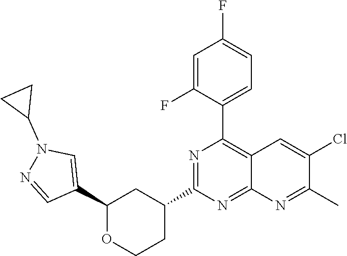 [SMILES: Cc1nc2nc([C@@H]3CCO[C@@H](c4cnn(C5CC5)c4)C3)nc(-c3ccc(F)cc3F)c2cc1Cl] |
| I-802 | 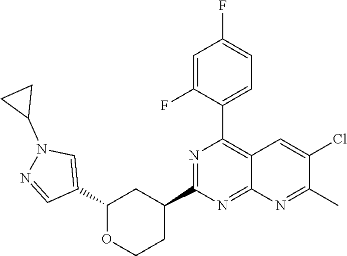 [SMILES: Cc1nc2nc([C@H]3CCO[C@H](c4cnn(C5CC5)c4)C3)nc(-c3ccc(F)cc3F)c2cc1Cl] |
| I-803 | 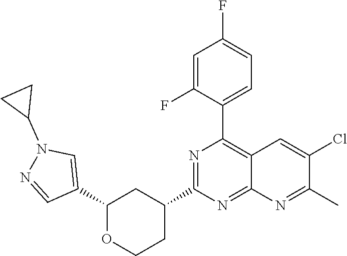 [SMILES: Cc1nc2nc([C@@H]3CCO[C@H](c4cnn(C5CC5)c4)C3)nc(-c3ccc(F)cc3F)c2cc1Cl] |
| I-804 | 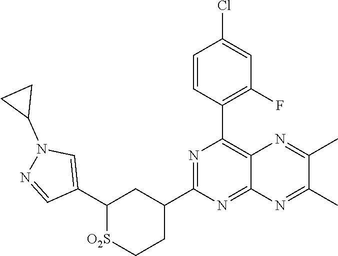 [SMILES: Cc1nc2nc(C3CCS(=O)(=O)C(c4cnn(C5CC5)c4)C3)nc(-c3ccc(Cl)cc3F)c2nc1C] |
| I-805 | 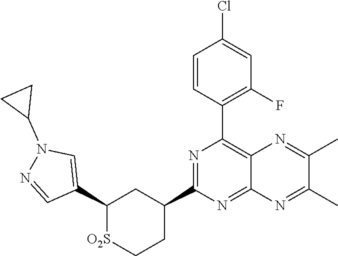 [SMILES: Cc1nc2nc([C@H]3CCS(=O)(=O)[C@@H](c4cnn(C5CC5)c4)C3)nc(-c3ccc(Cl)cc3F)c2nc1C] |
| I-806 | 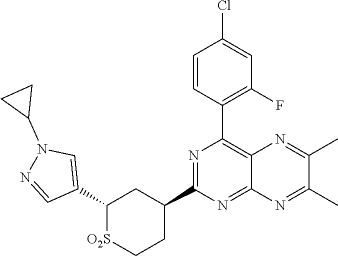 [SMILES: Cc1nc2nc([C@H]3CCS(=O)(=O)[C@H](c4cnn(C5CC5)c4)C3)nc(-c3ccc(Cl)cc3F)c2nc1C] |
| I-807 | 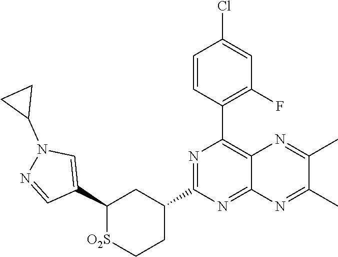 [SMILES: Cc1nc2nc([C@@H]3CCS(=O)(=O)[C@@H](c4cnn(C5CC5)c4)C3)nc(-c3ccc(Cl)cc3F)c2nc1C] |
| I-808 | 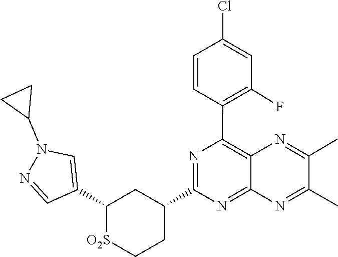 [SMILES: Cc1nc2nc([C@@H]3CCS(=O)(=O)[C@H](c4cnn(C5CC5)c4)C3)nc(-c3ccc(Cl)cc3F)c2nc1C] |
| I-809 | 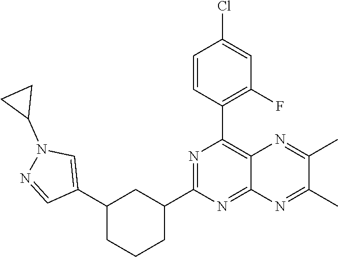 [SMILES: Cc1nc2nc(C3CCCC(c4cnn(C5CC5)c4)C3)nc(-c3ccc(Cl)cc3F)c2nc1C] |
| I-810 | 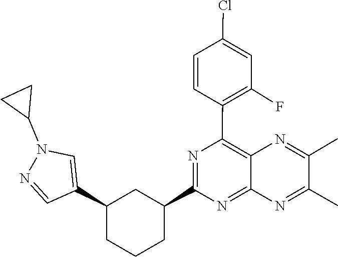 [SMILES: Cc1nc2nc([C@H]3CCC[C@@H](c4cnn(C5CC5)c4)C3)nc(-c3ccc(Cl)cc3F)c2nc1C] |
| I-811 | 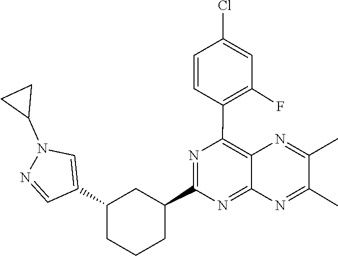 [SMILES: Cc1nc2nc([C@H]3CCC[C@H](c4cnn(C5CC5)c4)C3)nc(-c3ccc(Cl)cc3F)c2nc1C] |
| I-812 | 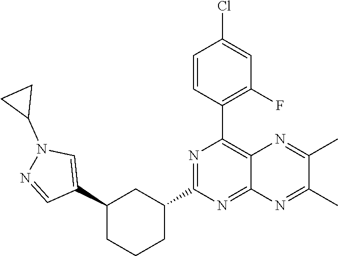 [SMILES: Cc1nc2nc([C@@H]3CCC[C@@H](c4cnn(C5CC5)c4)C3)nc(-c3ccc(Cl)cc3F)c2nc1C] |
| I-813 | 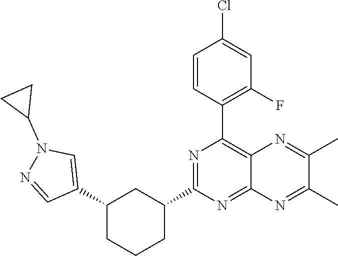 [SMILES: Cc1nc2nc([C@@H]3CCC[C@H](c4cnn(C5CC5)c4)C3)nc(-c3ccc(Cl)cc3F)c2nc1C] |
| I-814 | 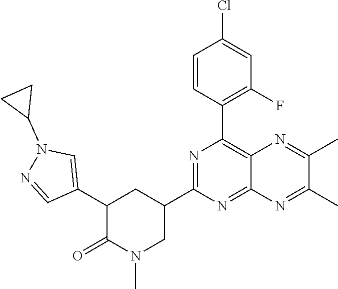 [SMILES: Cc1nc2nc(C3CC(c4cnn(C5CC5)c4)C(=O)N(C)C3)nc(-c3ccc(Cl)cc3F)c2nc1C] |
| I-815 | 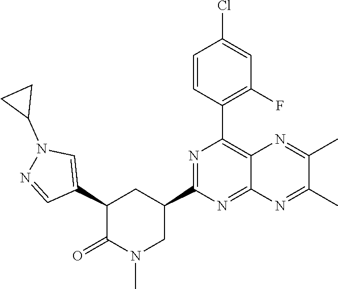 [SMILES: Cc1nc2nc([C@@H]3C[C@H](c4cnn(C5CC5)c4)C(=O)N(C)C3)nc(-c3ccc(Cl)cc3F)c2nc1C] |
| I-816 | 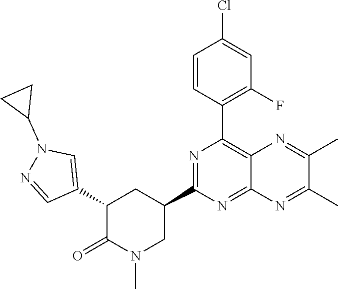 [SMILES: Cc1nc2nc([C@@H]3C[C@@H](c4cnn(C5CC5)c4)C(=O)N(C)C3)nc(-c3ccc(Cl)cc3F)c2nc1C] |
| I-817 | 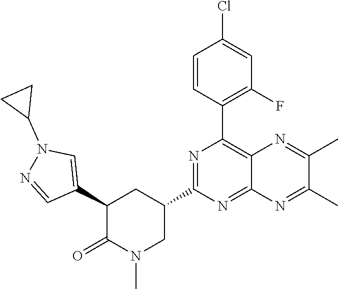 [SMILES: Cc1nc2nc([C@H]3C[C@H](c4cnn(C5CC5)c4)C(=O)N(C)C3)nc(-c3ccc(Cl)cc3F)c2nc1C] |
| I-818 | 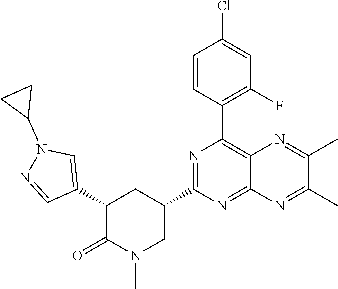 [SMILES: Cc1nc2nc([C@H]3C[C@@H](c4cnn(C5CC5)c4)C(=O)N(C)C3)nc(-c3ccc(Cl)cc3F)c2nc1C] |
| I-819 | 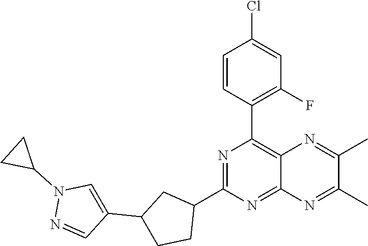 [SMILES: Cc1nc2nc(C3CCC(c4cnn(C5CC5)c4)C3)nc(-c3ccc(Cl)cc3F)c2nc1C] |
| I-820 | 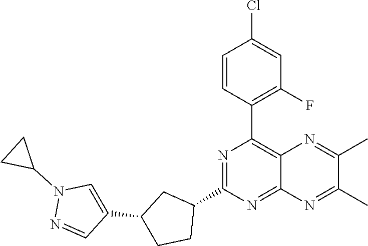 [SMILES: Cc1nc2nc([C@@H]3CC[C@H](c4cnn(C5CC5)c4)C3)nc(-c3ccc(Cl)cc3F)c2nc1C] |
| I-821 | 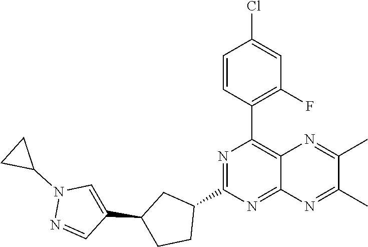 [SMILES: Cc1nc2nc([C@@H]3CC[C@@H](c4cnn(C5CC5)c4)C3)nc(-c3ccc(Cl)cc3F)c2nc1C] |
| I-822 | 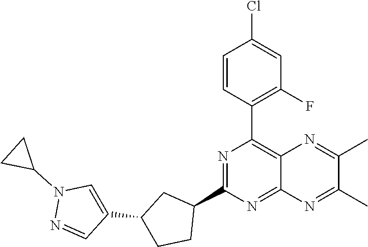 [SMILES: Cc1nc2nc([C@H]3CC[C@H](c4cnn(C5CC5)c4)C3)nc(-c3ccc(Cl)cc3F)c2nc1C] |
| I-823 | 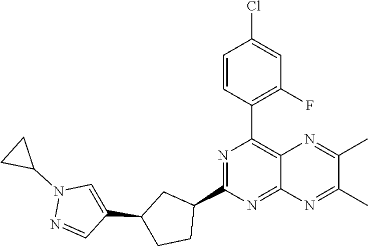 [SMILES: Cc1nc2nc([C@H]3CC[C@@H](c4cnn(C5CC5)c4)C3)nc(-c3ccc(Cl)cc3F)c2nc1C] |
| I-824 | 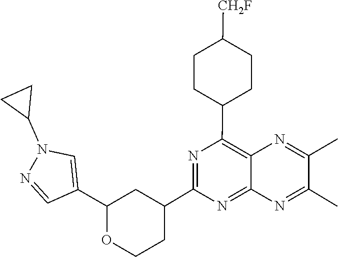 |
| I-825 | 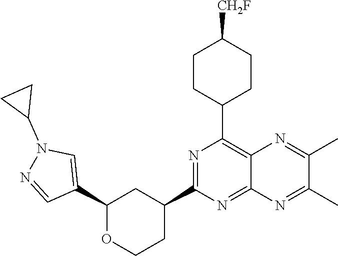 |
| I-826 | 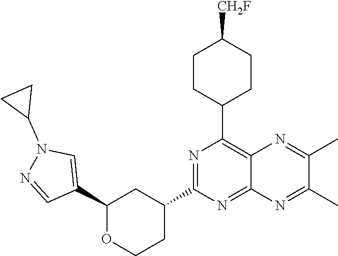 |
| I-827 | 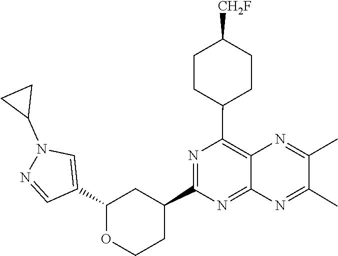 |
| I-828 | 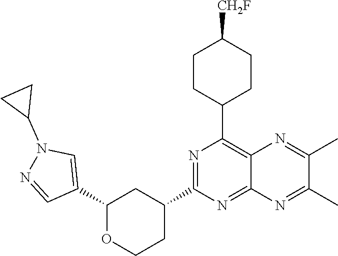 |
| I-829 | 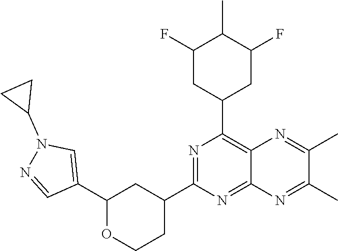 [SMILES: Cc1nc2nc(C3CCOC(c4cnn(C5CC5)c4)C3)nc(C3CC(F)C(C)C(F)C3)c2nc1C] |
| I-830 | 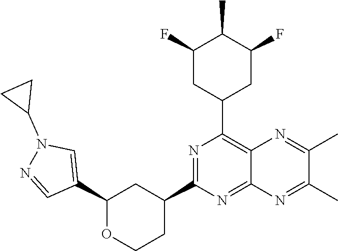 [SMILES: Cc1nc2nc([C@H]3CCO[C@@H](c4cnn(C5CC5)c4)C3)nc(C3C[C@@H](F)[C@@H](C)[C@@H](F)C3)c2nc1C] |
| I-831 | 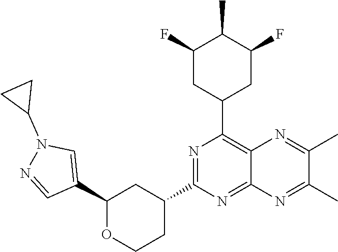 [SMILES: Cc1nc2nc([C@@H]3CCO[C@@H](c4cnn(C5CC5)c4)C3)nc(C3C[C@@H](F)[C@@H](C)[C@@H](F)C3)c2nc1C] |
| I-832 | 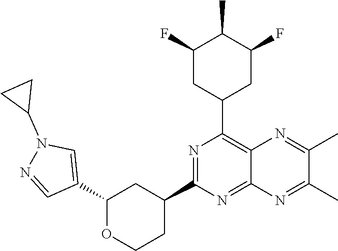 [SMILES: Cc1nc2nc([C@H]3CCO[C@H](c4cnn(C5CC5)c4)C3)nc(C3C[C@@H](F)[C@@H](C)[C@@H](F)C3)c2nc1C] |
| I-833 | 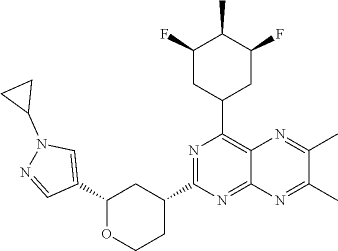 [SMILES: Cc1nc2nc([C@@H]3CCO[C@H](c4cnn(C5CC5)c4)C3)nc(C3C[C@@H](F)[C@@H](C)[C@@H](F)C3)c2nc1C] |
| I-834 | 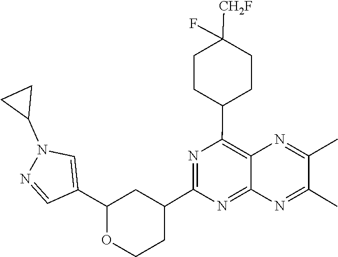 |
| I-835 | 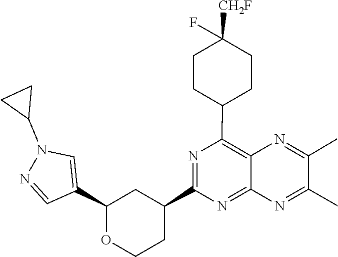 |
| I-836 | 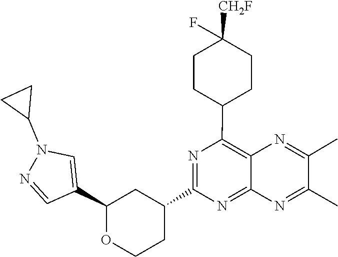 |
| I-837 | 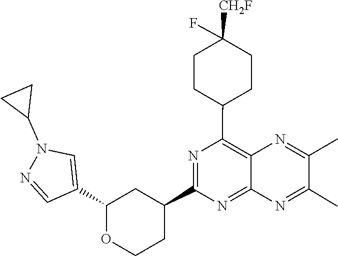 |
| I-838 | 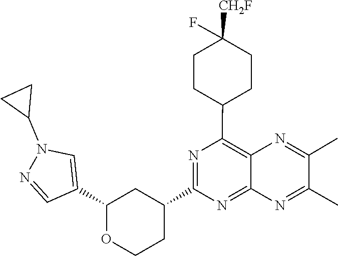 |
| I-839 | 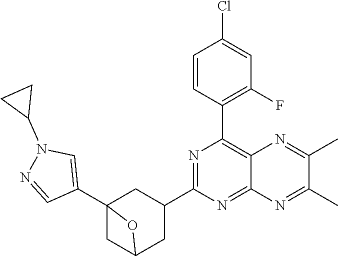 [SMILES: Cc1nc2nc(C3CC4CC(c5cnn(C6CC6)c5)(C3)O4)nc(-c3ccc(Cl)cc3F)c2nc1C] |
| I-840 | 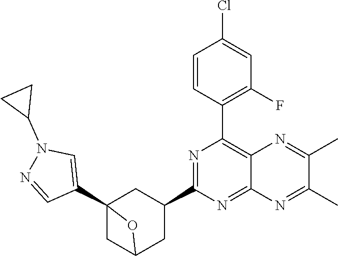 [SMILES: Cc1nc2nc([C@H]3CC4C[C@](c5cnn(C6CC6)c5)(C3)O4)nc(-c3ccc(Cl)cc3F)c2nc1C] |
| I-841 | 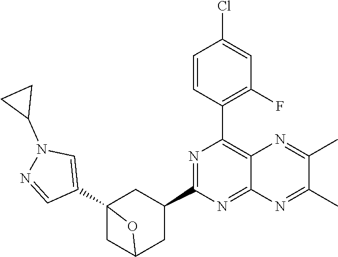 [SMILES: Cc1nc2nc([C@H]3CC4C[C@@](c5cnn(C6CC6)c5)(C3)O4)nc(-c3ccc(Cl)cc3F)c2nc1C] |
| I-842 | 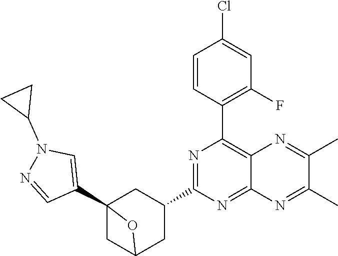 [SMILES: Cc1nc2nc([C@@H]3CC4C[C@](c5cnn(C6CC6)c5)(C3)O4)nc(-c3ccc(Cl)cc3F)c2nc1C] |
| I-843 | 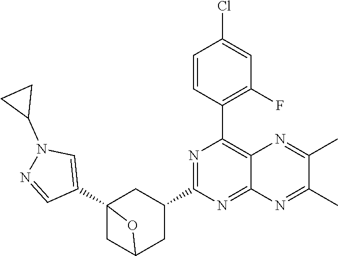 [SMILES: Cc1nc2nc([C@@H]3CC4C[C@@](c5cnn(C6CC6)c5)(C3)O4)nc(-c3ccc(Cl)cc3F)c2nc1C] |
| I-844 | 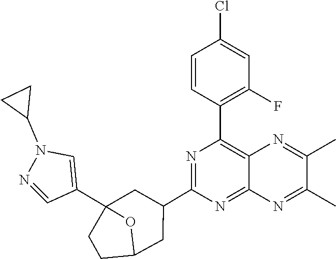 [SMILES: Cc1nc2nc(C3CC4CCC(c5cnn(C6CC6)c5)(C3)O4)nc(-c3ccc(Cl)cc3F)c2nc1C] |
| I-845 | 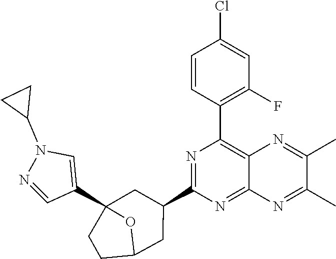 [SMILES: Cc1nc2nc([C@H]3CC4CC[C@](c5cnn(C6CC6)c5)(C3)O4)nc(-c3ccc(Cl)cc3F)c2nc1C] |
| I-846 | 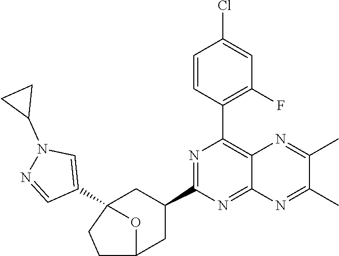 [SMILES: Cc1nc2nc([C@H]3CC4CC[C@@](c5cnn(C6CC6)c5)(C3)O4)nc(-c3ccc(Cl)cc3F)c2nc1C] |
| I-847 | 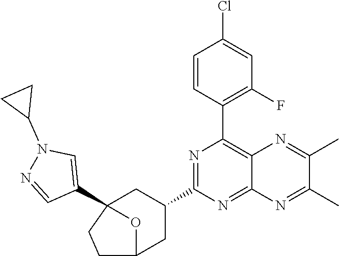 [SMILES: Cc1nc2nc([C@@H]3CC4CC[C@](c5cnn(C6CC6)c5)(C3)O4)nc(-c3ccc(Cl)cc3F)c2nc1C] |
| I-848 | 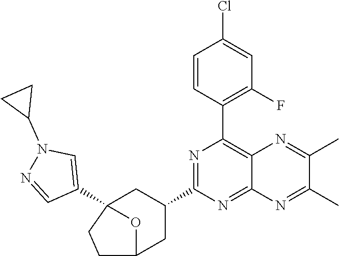 [SMILES: Cc1nc2nc([C@@H]3CC4CC[C@@](c5cnn(C6CC6)c5)(C3)O4)nc(-c3ccc(Cl)cc3F)c2nc1C] |
| I-849 | 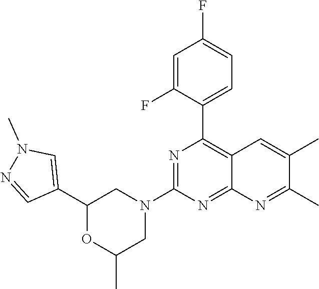 [SMILES: Cc1cc2c(-c3ccc(F)cc3F)nc(N3CC(C)OC(c4cnn(C)c4)C3)nc2nc1C] |
| I-850 | 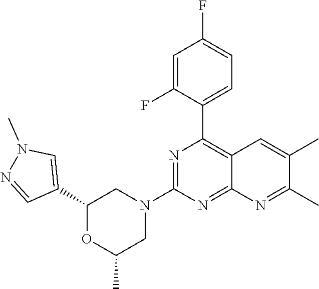 [SMILES: Cc1cc2c(-c3ccc(F)cc3F)nc(N3C[C@@H](c4cnn(C)c4)O[C@@H](C)C3)nc2nc1C] |
| I-851 | 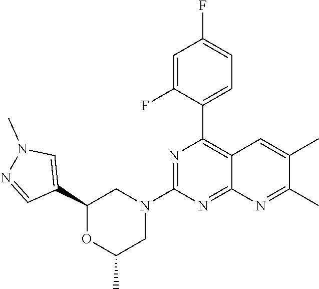 [SMILES: Cc1cc2c(-c3ccc(F)cc3F)nc(N3C[C@H](C)O[C@@H](c4cnn(C)c4)C3)nc2nc1C] |
| I-852 | 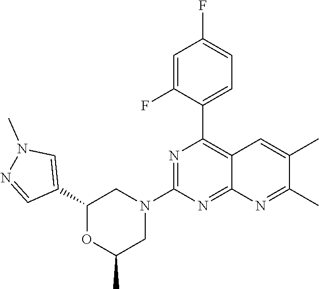 [SMILES: Cc1cc2c(-c3ccc(F)cc3F)nc(N3C[C@@H](C)O[C@H](c4cnn(C)c4)C3)nc2nc1C] |
| I-853 | 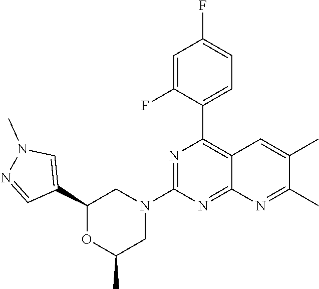 [SMILES: Cc1cc2c(-c3ccc(F)cc3F)nc(N3C[C@@H](C)O[C@@H](c4cnn(C)c4)C3)nc2nc1C] |
| I-854 | 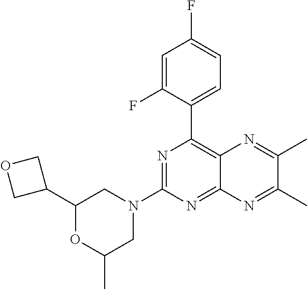 [SMILES: Cc1nc2nc(N3CC(C)OC(C4COC4)C3)nc(-c3ccc(F)cc3F)c2nc1C] |
| I-855 | 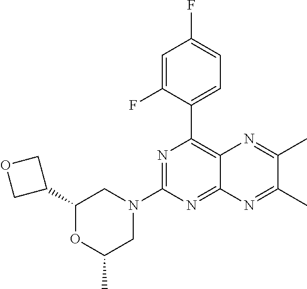 [SMILES: Cc1nc2nc(N3C[C@@H](C4COC4)O[C@@H](C)C3)nc(-c3ccc(F)cc3F)c2nc1C] |
| I-856 | 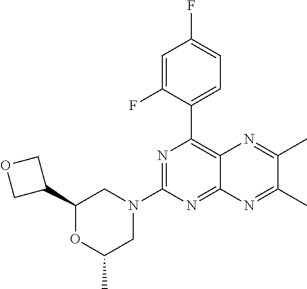 [SMILES: Cc1nc2nc(N3C[C@H](C)O[C@@H](C4COC4)C3)nc(-c3ccc(F)cc3F)c2nc1C] |
| I-857 | 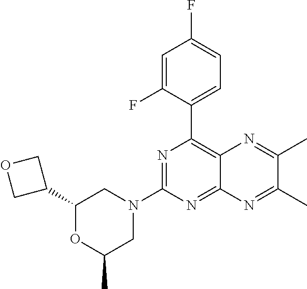 [SMILES: Cc1nc2nc(N3C[C@@H](C)O[C@H](C4COC4)C3)nc(-c3ccc(F)cc3F)c2nc1C] |
| I-858 | 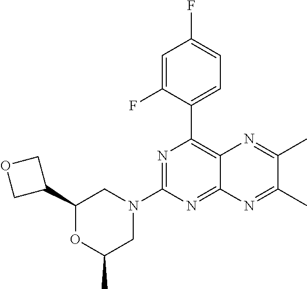 [SMILES: Cc1nc2nc(N3C[C@@H](C)O[C@@H](C4COC4)C3)nc(-c3ccc(F)cc3F)c2nc1C] |
| I-859 | 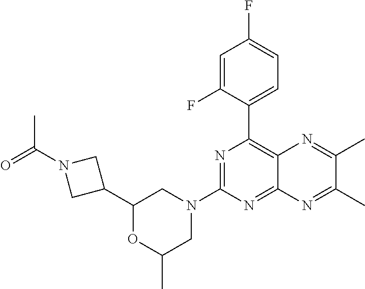 [SMILES: CC(=O)N1CC(C2CN(c3nc(-c4ccc(F)cc4F)c4nc(C)c(C)nc4n3)CC(C)O2)C1] |
| I-860 | 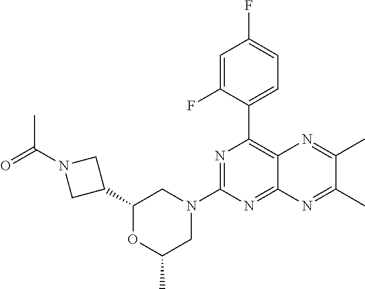 [SMILES: CC(=O)N1CC([C@@H]2CN(c3nc(-c4ccc(F)cc4F)c4nc(C)c(C)nc4n3)C[C@H](C)O2)C1] |
| I-861 | 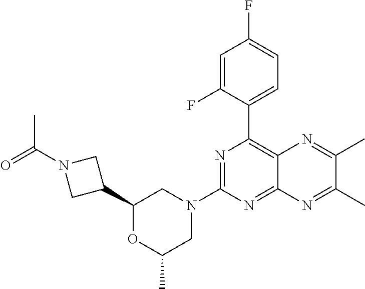 [SMILES: CC(=O)N1CC([C@H]2CN(c3nc(-c4ccc(F)cc4F)c4nc(C)c(C)nc4n3)C[C@H](C)O2)C1] |
| I-862 | 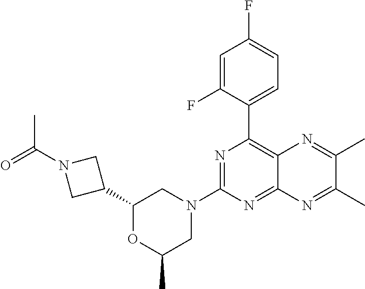 [SMILES: CC(=O)N1CC([C@@H]2CN(c3nc(-c4ccc(F)cc4F)c4nc(C)c(C)nc4n3)C[C@@H](C)O2)C1] |
| I-863 | 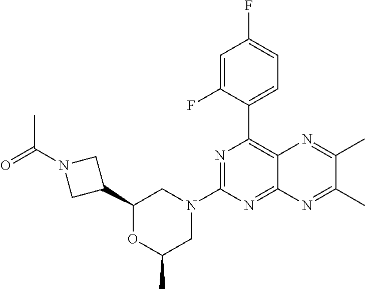 [SMILES: CC(=O)N1CC([C@H]2CN(c3nc(-c4ccc(F)cc4F)c4nc(C)c(C)nc4n3)C[C@@H](C)O2)C1] |
| I-864 | 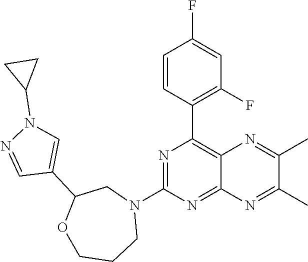 [SMILES: Cc1nc2nc(N3CCCOC(c4cnn(C5CC5)c4)C3)nc(-c3ccc(F)cc3F)c2nc1C] |
| I-865 | 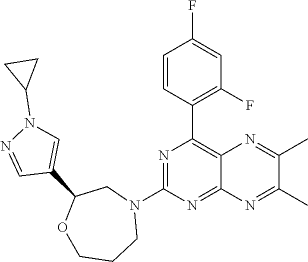 [SMILES: Cc1nc2nc(N3CCCO[C@@H](c4cnn(C5CC5)c4)C3)nc(-c3ccc(F)cc3F)c2nc1C] |
| I-866 | 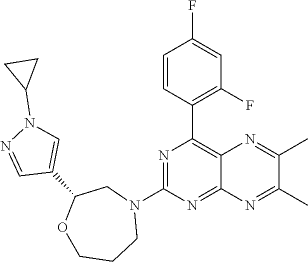 [SMILES: Cc1nc2nc(N3CCCO[C@H](c4cnn(C5CC5)c4)C3)nc(-c3ccc(F)cc3F)c2nc1C] |
| I-867 | 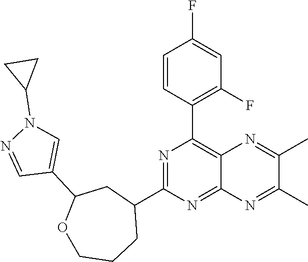 [SMILES: Cc1nc2nc(C3CCCOC(c4cnn(C5CC5)c4)C3)nc(-c3ccc(F)cc3F)c2nc1C] |
| I-868 | 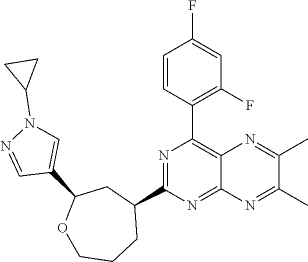 [SMILES: Cc1nc2nc([C@H]3CCCO[C@@H](c4cnn(C5CC5)c4)C3)nc(-c3ccc(F)cc3F)c2nc1C] |
| I-869 | 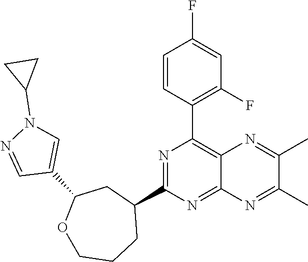 [SMILES: Cc1nc2nc([C@H]3CCCO[C@H](c4cnn(C5CC5)c4)C3)nc(-c3ccc(F)cc3F)c2nc1C] |
| I-870 | 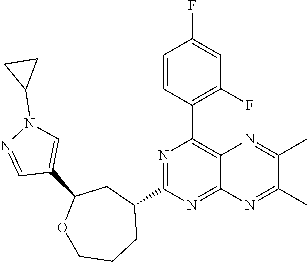 [SMILES: Cc1nc2nc([C@@H]3CCCO[C@@H](c4cnn(C5CC5)c4)C3)nc(-c3ccc(F)cc3F)c2nc1C] |
| I-871 | 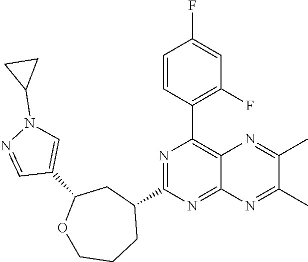 [SMILES: Cc1nc2nc([C@@H]3CCCO[C@H](c4cnn(C5CC5)c4)C3)nc(-c3ccc(F)cc3F)c2nc1C] |
| I-872 | 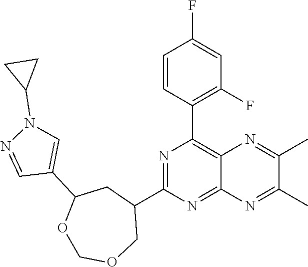 [SMILES: Cc1nc2nc(C3COCOC(c4cnn(C5CC5)c4)C3)nc(-c3ccc(F)cc3F)c2nc1C] |
| I-873 | 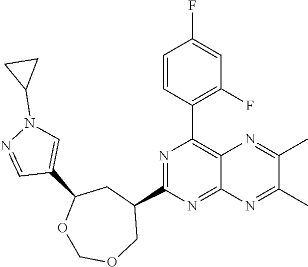 [SMILES: Cc1nc2nc([C@H]3COCO[C@@H](c4cnn(C5CC5)c4)C3)nc(-c3ccc(F)cc3F)c2nc1C] |
| I-874 | 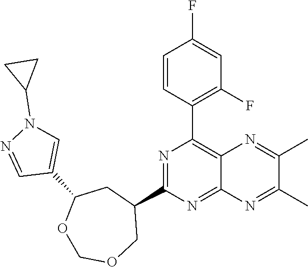 [SMILES: Cc1nc2nc([C@H]3COCO[C@H](c4cnn(C5CC5)c4)C3)nc(-c3ccc(F)cc3F)c2nc1C] |
| I-875 | 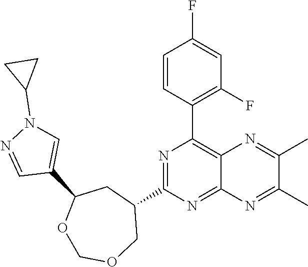 [SMILES: Cc1nc2nc([C@@H]3COCO[C@@H](c4cnn(C5CC5)c4)C3)nc(-c3ccc(F)cc3F)c2nc1C] |
| I-876 | 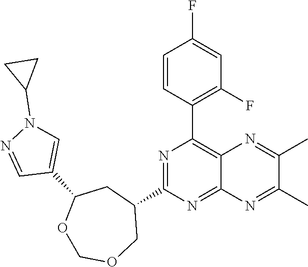 [SMILES: Cc1nc2nc([C@@H]3COCO[C@H](c4cnn(C5CC5)c4)C3)nc(-c3ccc(F)cc3F)c2nc1C] |
| I-877 | 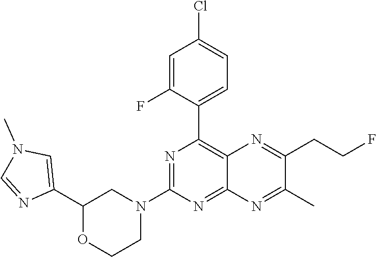 [SMILES: Cc1nc2nc(N3CCOC(c4cn(C)cn4)C3)nc(-c3ccc(Cl)cc3F)c2nc1CCF] |
| I-878 | 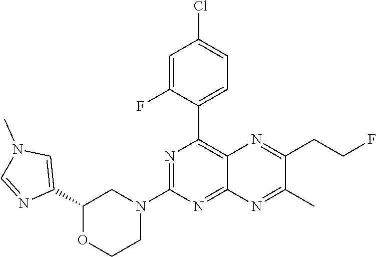 [SMILES: Cc1nc2nc(N3CCO[C@H](c4cn(C)cn4)C3)nc(-c3ccc(Cl)cc3F)c2nc1CCF] |
| I-879 | 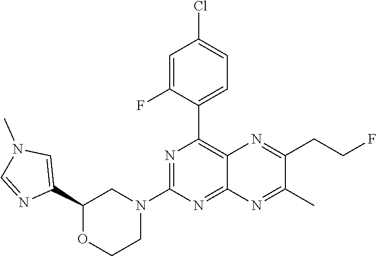 [SMILES: Cc1nc2nc(N3CCO[C@@H](c4cn(C)cn4)C3)nc(-c3ccc(Cl)cc3F)c2nc1CCF] |
| I-880 | 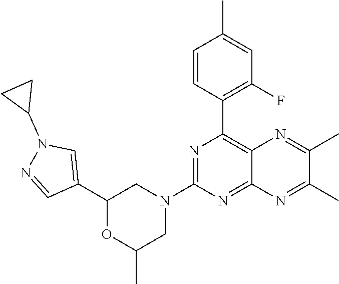 [SMILES: Cc1ccc(-c2nc(N3CC(C)OC(c4cnn(C5CC5)c4)C3)nc3nc(C)c(C)nc23)c(F)c1] |
| I-881 | 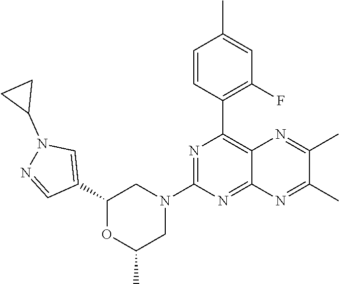 [SMILES: Cc1ccc(-c2nc(N3C[C@@H](c4cnn(C5CC5)c4)O[C@@H](C)C3)nc3nc(C)c(C)nc23)c(F)c1] |
| I-882 | 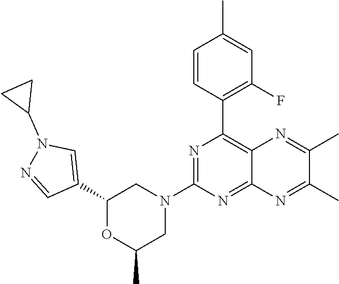 [SMILES: Cc1ccc(-c2nc(N3C[C@@H](C)O[C@H](c4cnn(C5CC5)c4)C3)nc3nc(C)c(C)nc23)c(F)c1] |
| I-883 | 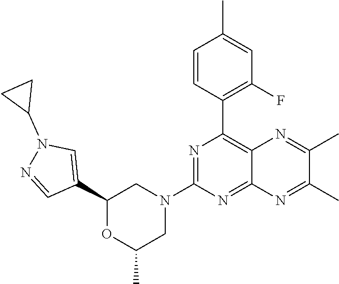 [SMILES: Cc1ccc(-c2nc(N3C[C@H](C)O[C@@H](c4cnn(C5CC5)c4)C3)nc3nc(C)c(C)nc23)c(F)c1] |
| I-884 | 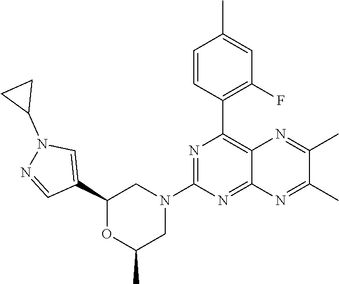 [SMILES: Cc1ccc(-c2nc(N3C[C@@H](C)O[C@@H](c4cnn(C5CC5)c4)C3)nc3nc(C)c(C)nc23)c(F)c1] |
| I-885 | 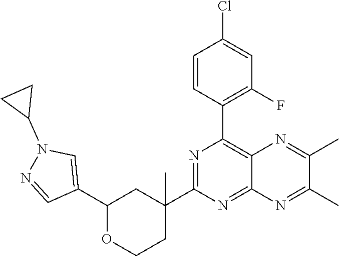 [SMILES: Cc1nc2nc(C3(C)CCOC(c4cnn(C5CC5)c4)C3)nc(-c3ccc(Cl)cc3F)c2nc1C] |
| I-886 | 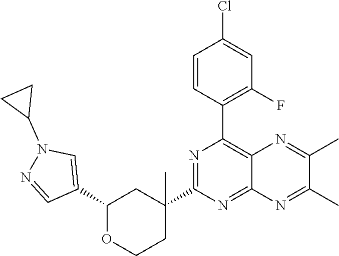 [SMILES: Cc1nc2nc([C@]3(C)CCO[C@H](c4cnn(C5CC5)c4)C3)nc(-c3ccc(Cl)cc3F)c2nc1C] |
| I-887 | 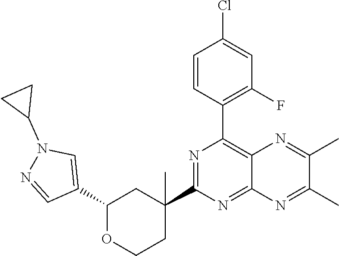 [SMILES: Cc1nc2nc([C@@]3(C)CCO[C@H](c4cnn(C5CC5)c4)C3)nc(-c3ccc(Cl)cc3F)c2nc1C] |
| I-888 | 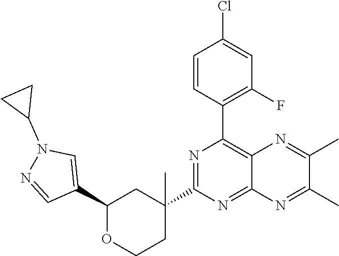 [SMILES: Cc1nc2nc([C@]3(C)CCO[C@@H](c4cnn(C5CC5)c4)C3)nc(-c3ccc(Cl)cc3F)c2nc1C] |
| I-889 | 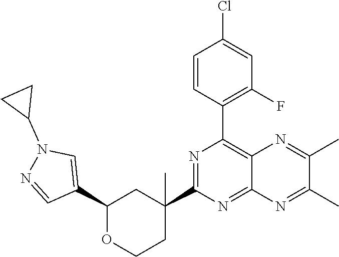 [SMILES: Cc1nc2nc([C@@]3(C)CCO[C@@H](c4cnn(C5CC5)c4)C3)nc(-c3ccc(Cl)cc3F)c2nc1C] |
| I-890 | 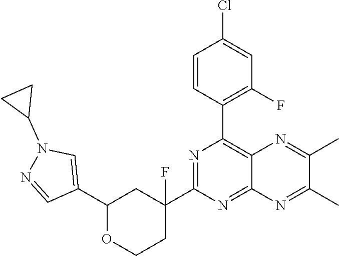 [SMILES: Cc1nc2nc(C3(F)CCOC(c4cnn(C5CC5)c4)C3)nc(-c3ccc(Cl)cc3F)c2nc1C] |
| I-891 | 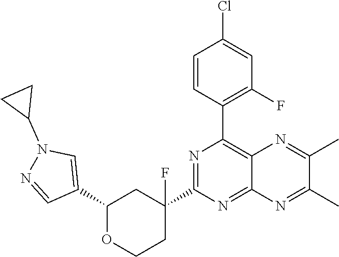 [SMILES: Cc1nc2nc([C@]3(F)CCO[C@H](c4cnn(C5CC5)c4)C3)nc(-c3ccc(Cl)cc3F)c2nc1C] |
| I-892 | 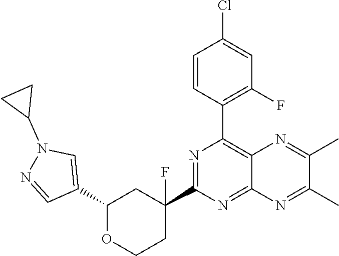 [SMILES: Cc1nc2nc([C@@]3(F)CCO[C@H](c4cnn(C5CC5)c4)C3)nc(-c3ccc(Cl)cc3F)c2nc1C] |
| I-893 | 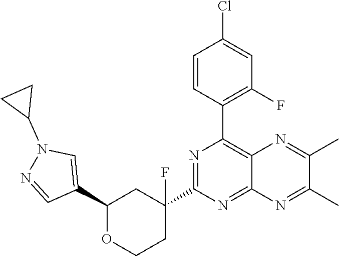 [SMILES: Cc1nc2nc([C@]3(F)CCO[C@@H](c4cnn(C5CC5)c4)C3)nc(-c3ccc(Cl)cc3F)c2nc1C] |
| I-894 | 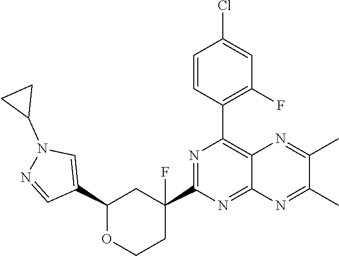 [SMILES: Cc1nc2nc([C@@]3(F)CCO[C@@H](c4cnn(C5CC5)c4)C3)nc(-c3ccc(Cl)cc3F)c2nc1C] |
| I-895 | 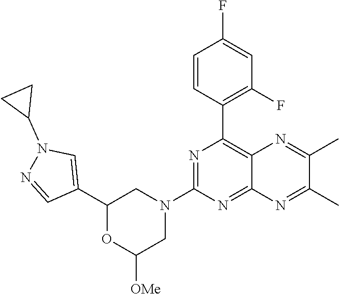 [SMILES: COC1CN(c2nc(-c3ccc(F)cc3F)c3nc(C)c(C)nc3n2)CC(c2cnn(C3CC3)c2)O1] |
| I-896 | 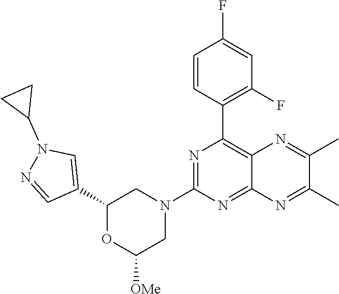 [SMILES: CO[C@H]1CN(c2nc(-c3ccc(F)cc3F)c3nc(C)c(C)nc3n2)C[C@@H](c2cnn(C3CC3)c2)O1] |
| I-897 | 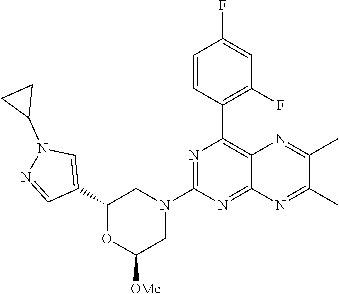 [SMILES: CO[C@@H]1CN(c2nc(-c3ccc(F)cc3F)c3nc(C)c(C)nc3n2)C[C@@H](c2cnn(C3CC3)c2)O1] |
| I-898 | 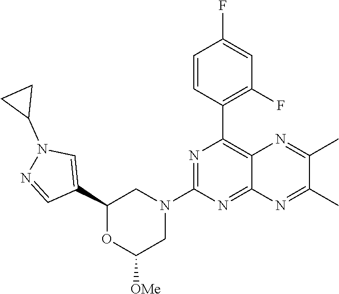 [SMILES: CO[C@H]1CN(c2nc(-c3ccc(F)cc3F)c3nc(C)c(C)nc3n2)C[C@H](c2cnn(C3CC3)c2)O1] |
| I-899 | 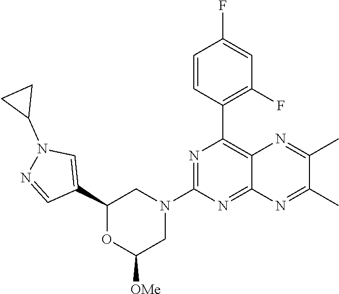 [SMILES: CO[C@@H]1CN(c2nc(-c3ccc(F)cc3F)c3nc(C)c(C)nc3n2)C[C@H](c2cnn(C3CC3)c2)O1] |
| I-900 | 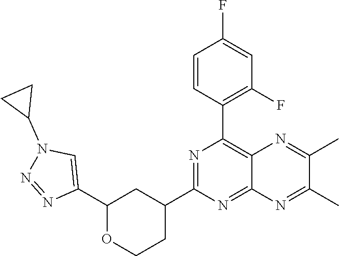 [SMILES: Cc1nc2nc(C3CCOC(c4cn(C5CC5)nn4)C3)nc(-c3ccc(F)cc3F)c2nc1C] |
| I-901 | 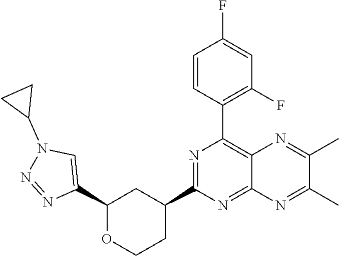 [SMILES: Cc1nc2nc([C@H]3CCO[C@@H](c4cn(C5CC5)nn4)C3)nc(-c3ccc(F)cc3F)c2nc1C] |
| I-902 | 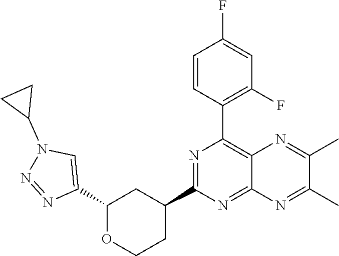 [SMILES: Cc1nc2nc([C@H]3CCO[C@H](c4cn(C5CC5)nn4)C3)nc(-c3ccc(F)cc3F)c2nc1C] |
| I-903 | 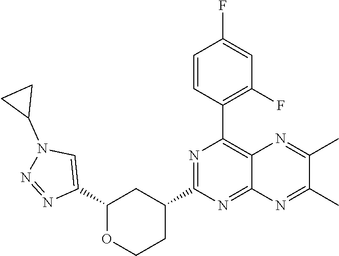 [SMILES: Cc1nc2nc([C@@H]3CCO[C@H](c4cn(C5CC5)nn4)C3)nc(-c3ccc(F)cc3F)c2nc1C] |
| I-904 | 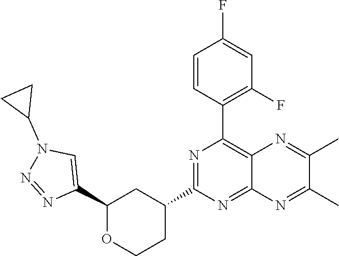 [SMILES: Cc1nc2nc([C@@H]3CCO[C@@H](c4cn(C5CC5)nn4)C3)nc(-c3ccc(F)cc3F)c2nc1C] |
| I-905 | 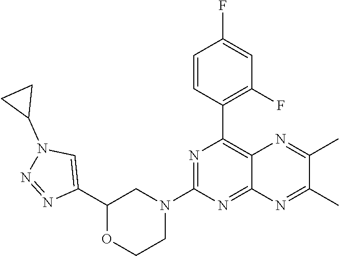 [SMILES: Cc1nc2nc(N3CCOC(c4cn(C5CC5)nn4)C3)nc(-c3ccc(F)cc3F)c2nc1C] |
| I-906 | 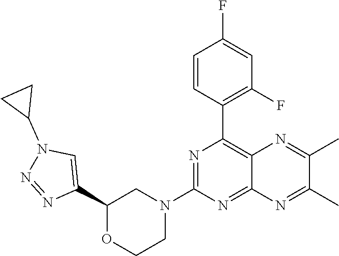 [SMILES: Cc1nc2nc(N3CCO[C@@H](c4cn(C5CC5)nn4)C3)nc(-c3ccc(F)cc3F)c2nc1C] |
| I-907 | 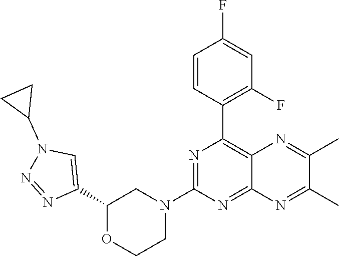 [SMILES: Cc1nc2nc(N3CCO[C@H](c4cn(C5CC5)nn4)C3)nc(-c3ccc(F)cc3F)c2nc1C] |
| I-908 | 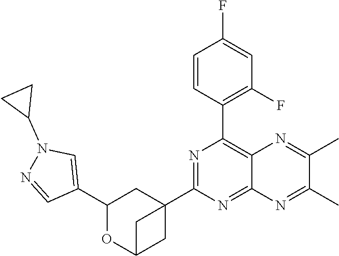 [SMILES: Cc1nc2nc(C34CC(C3)OC(c3cnn(C5CC5)c3)C4)nc(-c3ccc(F)cc3F)c2nc1C] |
| I-909 | 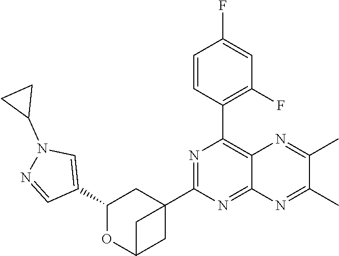 [SMILES: Cc1nc2nc(C34CC(C3)O[C@H](c3cnn(C5CC5)c3)C4)nc(-c3ccc(F)cc3F)c2nc1C] |
| I-910 | 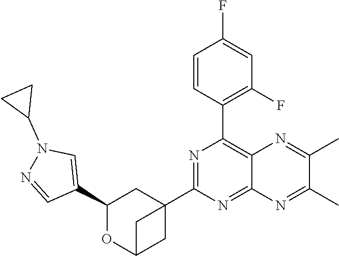 [SMILES: Cc1nc2nc(C34CC(C3)O[C@@H](c3cnn(C5CC5)c3)C4)nc(-c3ccc(F)cc3F)c2nc1C] |
| I-911 | 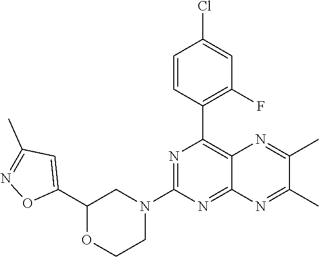 [SMILES: Cc1cc(C2CN(c3nc(-c4ccc(Cl)cc4F)c4nc(C)c(C)nc4n3)CCO2)on1] |
| I-912 | 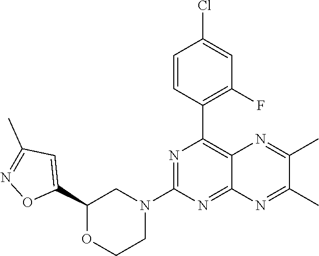 [SMILES: Cc1cc([C@H]2CN(c3nc(-c4ccc(Cl)cc4F)c4nc(C)c(C)nc4n3)CCO2)on1] |
| I-913 | 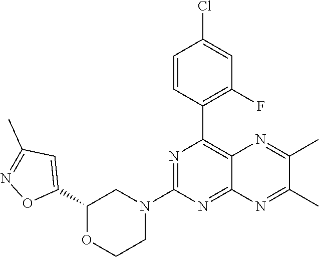 [SMILES: Cc1cc([C@@H]2CN(c3nc(-c4ccc(Cl)cc4F)c4nc(C)c(C)nc4n3)CCO2)on1] |
| I-914 | 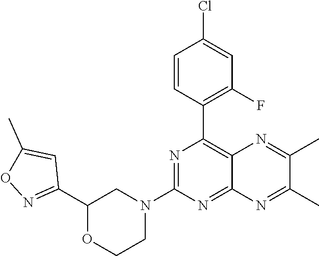 [SMILES: Cc1cc(C2CN(c3nc(-c4ccc(Cl)cc4F)c4nc(C)c(C)nc4n3)CCO2)no1] |
| I-915 | 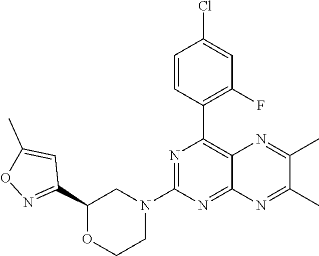 [SMILES: Cc1cc([C@H]2CN(c3nc(-c4ccc(Cl)cc4F)c4nc(C)c(C)nc4n3)CCO2)no1] |
| I-916 | 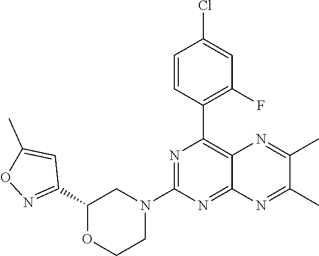 [SMILES: Cc1cc([C@@H]2CN(c3nc(-c4ccc(Cl)cc4F)c4nc(C)c(C)nc4n3)CCO2)no1] |
| I-917 | 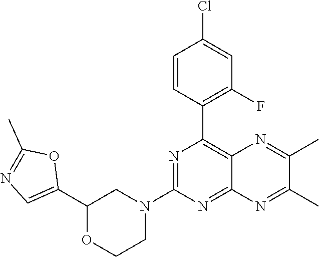 [SMILES: Cc1ncc(C2CN(c3nc(-c4ccc(Cl)cc4F)c4nc(C)c(C)nc4n3)CCO2)o1] |
| I-918 | 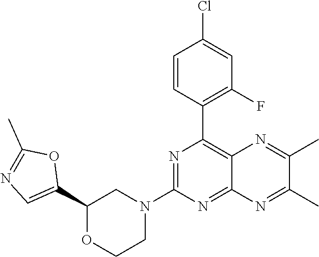 [SMILES: Cc1ncc([C@H]2CN(c3nc(-c4ccc(Cl)cc4F)c4nc(C)c(C)nc4n3)CCO2)o1] |
| I-919 | 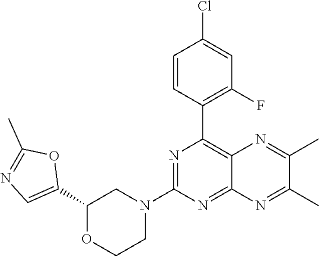 [SMILES: Cc1ncc([C@@H]2CN(c3nc(-c4ccc(Cl)cc4F)c4nc(C)c(C)nc4n3)CCO2)o1] |
| I-920 | 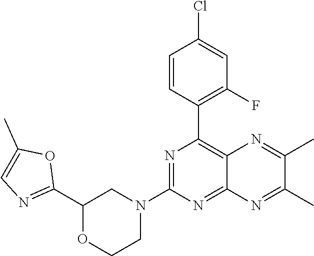 [SMILES: Cc1cnc(C2CN(c3nc(-c4ccc(Cl)cc4F)c4nc(C)c(C)nc4n3)CCO2)o1] |
| I-921 | 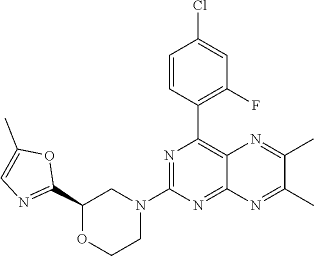 [SMILES: Cc1cnc([C@H]2CN(c3nc(-c4ccc(Cl)cc4F)c4nc(C)c(C)nc4n3)CCO2)o1] |
| I-922 | 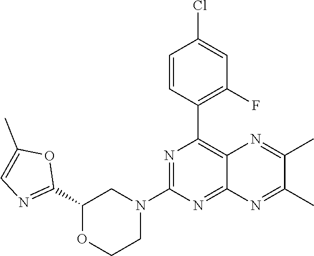 [SMILES: Cc1cnc([C@@H]2CN(c3nc(-c4ccc(Cl)cc4F)c4nc(C)c(C)nc4n3)CCO2)o1] |
| I-923 | 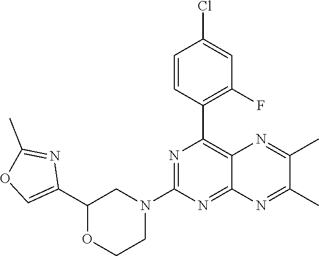 [SMILES: Cc1nc(C2CN(c3nc(-c4ccc(Cl)cc4F)c4nc(C)c(C)nc4n3)CCO2)co1] |
| I-924 | 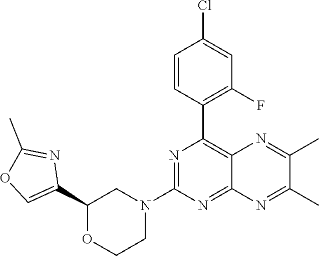 [SMILES: Cc1nc([C@H]2CN(c3nc(-c4ccc(Cl)cc4F)c4nc(C)c(C)nc4n3)CCO2)co1] |
| I-925 | 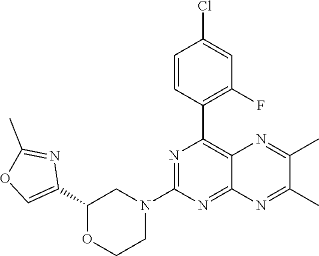 [SMILES: Cc1nc([C@@H]2CN(c3nc(-c4ccc(Cl)cc4F)c4nc(C)c(C)nc4n3)CCO2)co1] |
| I-926 | 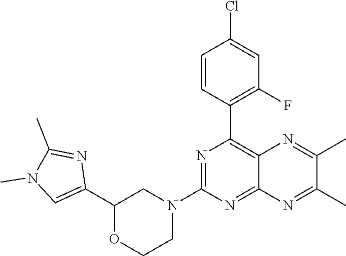 |
| I-927 | 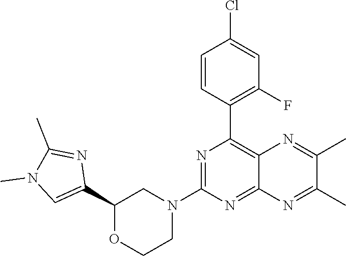 |
| I-928 | 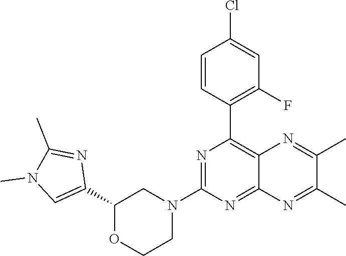 |
| I-929 | 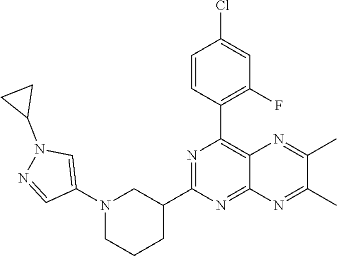 [SMILES: Cc1nc2nc(C3CCCN(c4cnn(C5CC5)c4)C3)nc(-c3ccc(Cl)cc3F)c2nc1C] |
| I-930 | 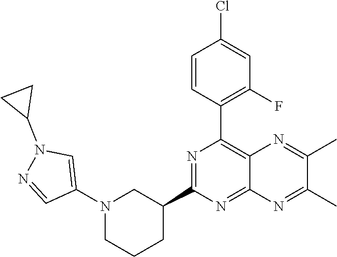 [SMILES: Cc1nc2nc([C@H]3CCCN(c4cnn(C5CC5)c4)C3)nc(-c3ccc(Cl)cc3F)c2nc1C] |
| I-931 | 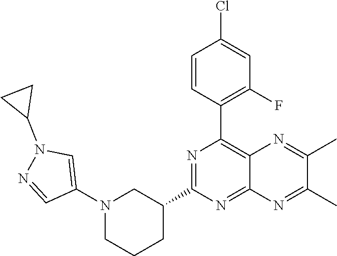 [SMILES: Cc1nc2nc([C@@H]3CCCN(c4cnn(C5CC5)c4)C3)nc(-c3ccc(Cl)cc3F)c2nc1C] |
| I-932 | 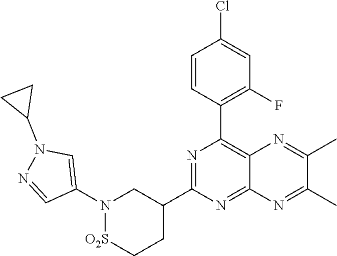 [SMILES: Cc1nc2nc(C3CCS(=O)(=O)N(c4cnn(C5CC5)c4)C3)nc(-c3ccc(Cl)cc3F)c2nc1C] |
| I-933 | 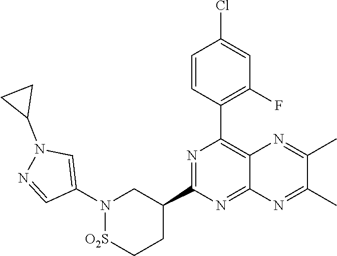 [SMILES: Cc1nc2nc([C@H]3CCS(=O)(=O)N(c4cnn(C5CC5)c4)C3)nc(-c3ccc(Cl)cc3F)c2nc1C] |
| I-934 | 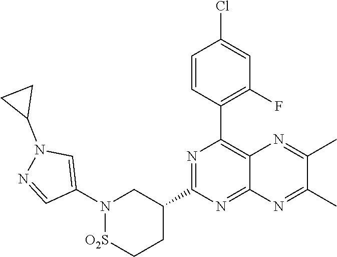 [SMILES: Cc1nc2nc([C@@H]3CCS(=O)(=O)N(c4cnn(C5CC5)c4)C3)nc(-c3ccc(Cl)cc3F)c2nc1C] |
| I-935 | 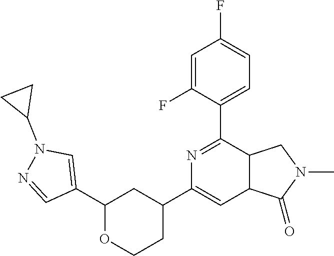 [SMILES: CN1CC2C(c3ccc(F)cc3F)=NC(C3CCOC(c4cnn(C5CC5)c4)C3)=CC2C1=O] |
| I-936 | 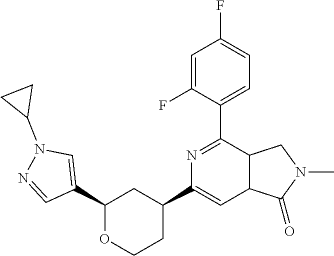 [SMILES: CN1CC2C(c3ccc(F)cc3F)=NC([C@H]3CCO[C@@H](c4cnn(C5CC5)c4)C3)=CC2C1=O] |
| I-937 | 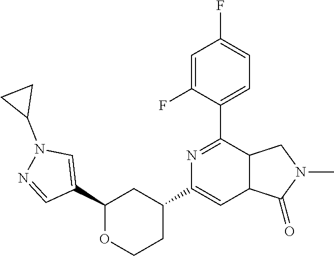 [SMILES: CN1CC2C(c3ccc(F)cc3F)=NC([C@@H]3CCO[C@@H](c4cnn(C5CC5)c4)C3)=CC2C1=O] |
| I-938 | 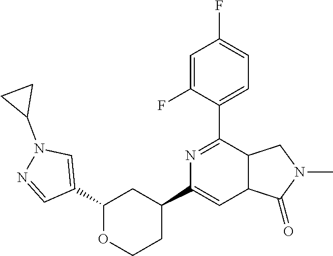 [SMILES: CN1CC2C(c3ccc(F)cc3F)=NC([C@H]3CCO[C@H](c4cnn(C5CC5)c4)C3)=CC2C1=O] |
| I-939 | 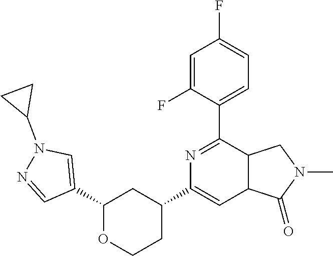 [SMILES: CN1CC2C(c3ccc(F)cc3F)=NC([C@@H]3CCO[C@H](c4cnn(C5CC5)c4)C3)=CC2C1=O] |
| I-940 | 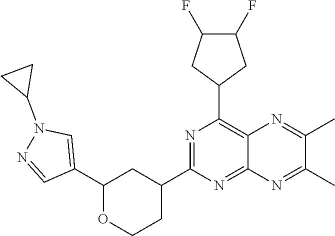 [SMILES: Cc1nc2nc(C3CCOC(c4cnn(C5CC5)c4)C3)nc(C3CC(F)C(F)C3)c2nc1C] |
| I-941 | 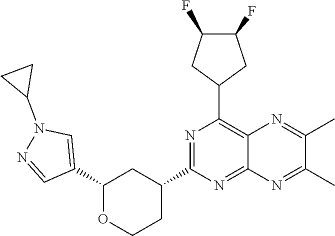 [SMILES: Cc1nc2nc([C@@H]3CCO[C@H](c4cnn(C5CC5)c4)C3)nc(C3C[C@@H](F)[C@@H](F)C3)c2nc1C] |
| I-942 | 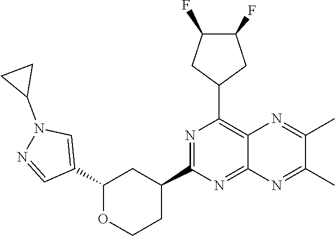 [SMILES: Cc1nc2nc([C@H]3CCO[C@H](c4cnn(C5CC5)c4)C3)nc(C3C[C@@H](F)[C@@H](F)C3)c2nc1C] |
| I-943 | 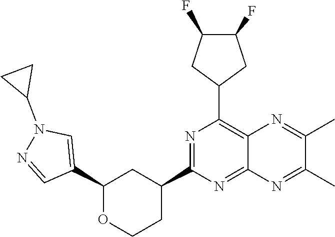 [SMILES: Cc1nc2nc([C@H]3CCO[C@@H](c4cnn(C5CC5)c4)C3)nc(C3C[C@@H](F)[C@@H](F)C3)c2nc1C] |
| I-944 | 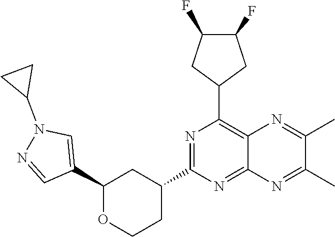 [SMILES: Cc1nc2nc([C@@H]3CCO[C@@H](c4cnn(C5CC5)c4)C3)nc(C3C[C@@H](F)[C@@H](F)C3)c2nc1C] |
| I-945 | 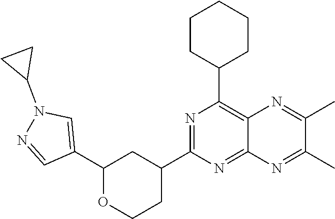 [SMILES: Cc1nc2nc(C3CCOC(c4cnn(C5CC5)c4)C3)nc(C3CCCCC3)c2nc1C] |
| I-946 | 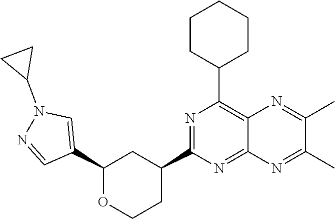 [SMILES: Cc1nc2nc([C@H]3CCO[C@@H](c4cnn(C5CC5)c4)C3)nc(C3CCCCC3)c2nc1C] |
| I-947 | 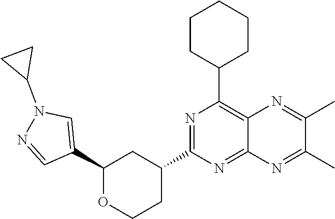 [SMILES: Cc1nc2nc([C@@H]3CCO[C@@H](c4cnn(C5CC5)c4)C3)nc(C3CCCCC3)c2nc1C] |
| I-948 | 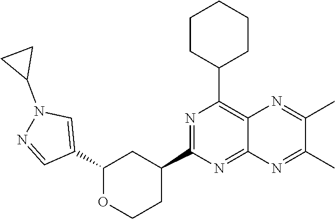 [SMILES: Cc1nc2nc([C@H]3CCO[C@H](c4cnn(C5CC5)c4)C3)nc(C3CCCCC3)c2nc1C] |
| I-949 | 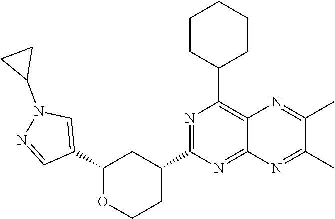 [SMILES: Cc1nc2nc([C@@H]3CCO[C@H](c4cnn(C5CC5)c4)C3)nc(C3CCCCC3)c2nc1C] |
| I-950 | 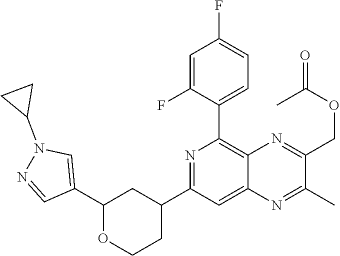 [SMILES: CC(=O)OCc1nc2c(-c3ccc(F)cc3F)nc(C3CCOC(c4cnn(C5CC5)c4)C3)cc2nc1C] |
| I-951 | 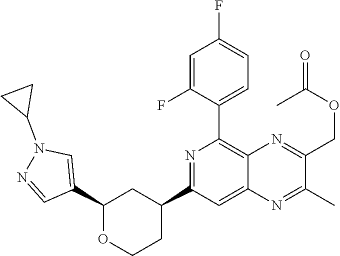 [SMILES: CC(=O)OCc1nc2c(-c3ccc(F)cc3F)nc([C@H]3CCO[C@@H](c4cnn(C5CC5)c4)C3)cc2nc1C] |
| I-952 | 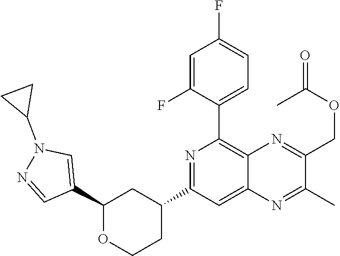 [SMILES: CC(=O)OCc1nc2c(-c3ccc(F)cc3F)nc([C@@H]3CCO[C@@H](c4cnn(C5CC5)c4)C3)cc2nc1C] |
| I-953 | 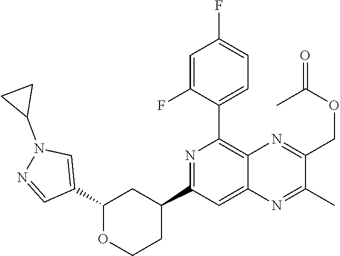 [SMILES: CC(=O)OCc1nc2c(-c3ccc(F)cc3F)nc([C@H]3CCO[C@H](c4cnn(C5CC5)c4)C3)cc2nc1C] |
| I-954 | 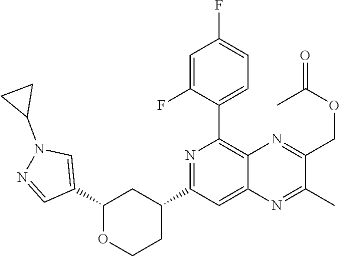 [SMILES: CC(=O)OCc1nc2c(-c3ccc(F)cc3F)nc([C@@H]3CCO[C@H](c4cnn(C5CC5)c4)C3)cc2nc1C] |
| I-955 | 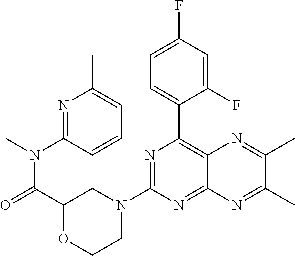 [SMILES: Cc1cccc(N(C)C(=O)C2CN(c3nc(-c4ccc(F)cc4F)c4nc(C)c(C)nc4n3)CCO2)n1] |
| I-956 | 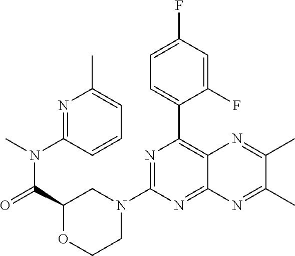 [SMILES: Cc1cccc(N(C)C(=O)[C@H]2CN(c3nc(-c4ccc(F)cc4F)c4nc(C)c(C)nc4n3)CCO2)n1] |
| I-957 | 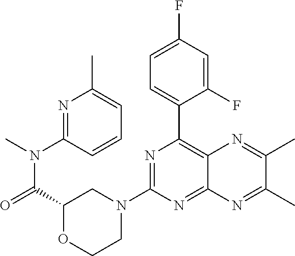 [SMILES: Cc1cccc(N(C)C(=O)[C@@H]2CN(c3nc(-c4ccc(F)cc4F)c4nc(C)c(C)nc4n3)CCO2)n1] |
| I-958 | 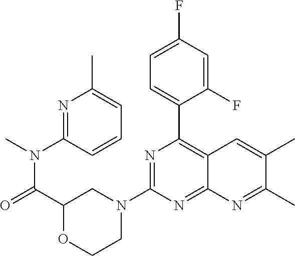 [SMILES: Cc1cccc(N(C)C(=O)C2CN(c3nc(-c4ccc(F)cc4F)c4cc(C)c(C)nc4n3)CCO2)n1] |
| I-959 | 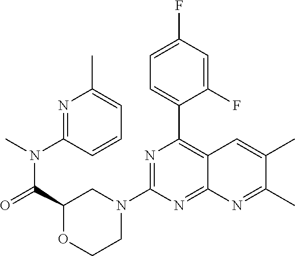 [SMILES: Cc1cccc(N(C)C(=O)[C@H]2CN(c3nc(-c4ccc(F)cc4F)c4cc(C)c(C)nc4n3)CCO2)n1] |
| I-960 | 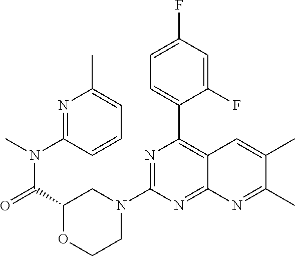 [SMILES: Cc1cccc(N(C)C(=O)[C@@H]2CN(c3nc(-c4ccc(F)cc4F)c4cc(C)c(C)nc4n3)CCO2)n1] |
| I-961 | 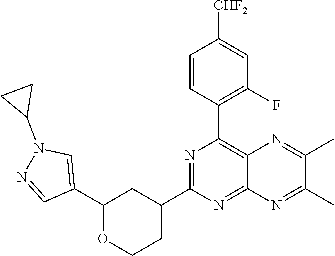 [SMILES: Cc1nc2nc(C3CCOC(c4cnn(C5CC5)c4)C3)nc(-c3ccc(C(F)F)cc3F)c2nc1C] |
| I-962 | 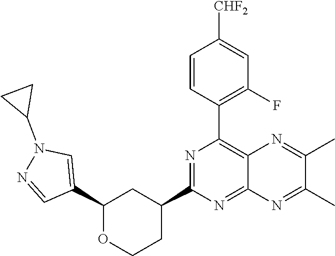 [SMILES: Cc1nc2nc([C@H]3CCO[C@@H](c4cnn(C5CC5)c4)C3)nc(-c3ccc(C(F)F)cc3F)c2nc1C] |
| I-963 | 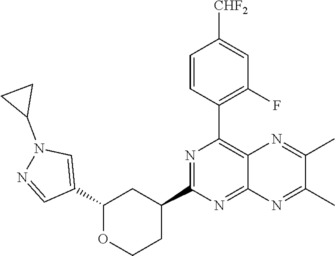 [SMILES: Cc1nc2nc([C@H]3CCO[C@H](c4cnn(C5CC5)c4)C3)nc(-c3ccc(C(F)F)cc3F)c2nc1C] |
| I-964 | 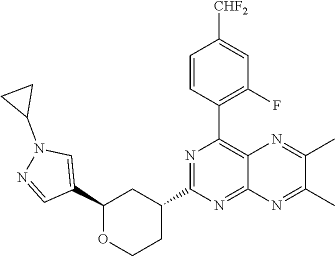 [SMILES: Cc1nc2nc([C@@H]3CCO[C@@H](c4cnn(C5CC5)c4)C3)nc(-c3ccc(C(F)F)cc3F)c2nc1C] |
| I-965 | 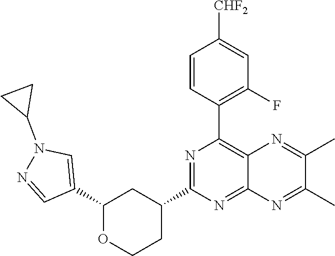 [SMILES: Cc1nc2nc([C@@H]3CCO[C@H](c4cnn(C5CC5)c4)C3)nc(-c3ccc(C(F)F)cc3F)c2nc1C] |
| I-966 | 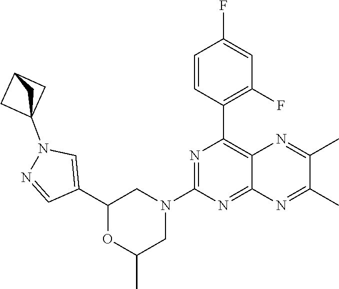 |
| I-967 | 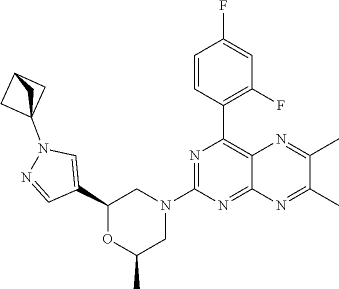 |
| I-968 | 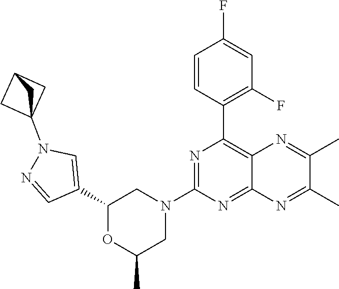 |
| I-969 | 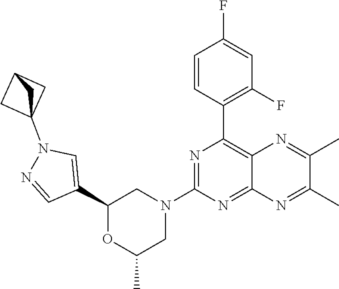 |
| I-970 | 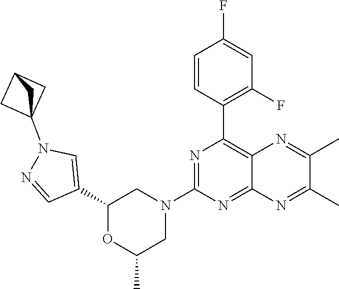 |
| I-971 | 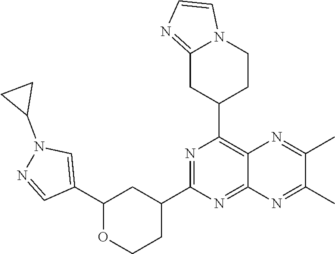 [SMILES: Cc1nc2nc(C3CCOC(c4cnn(C5CC5)c4)C3)nc(C3CCn4ccnc4C3)c2nc1C] |
| I-972 | 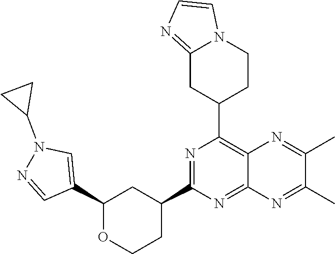 [SMILES: Cc1nc2nc([C@H]3CCO[C@@H](c4cnn(C5CC5)c4)C3)nc(C3CCn4ccnc4C3)c2nc1C] |
| I-973 | 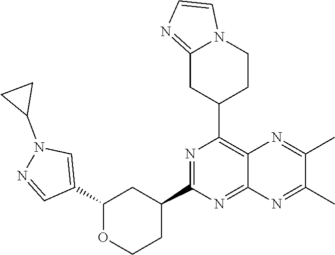 [SMILES: Cc1nc2nc([C@H]3CCO[C@H](c4cnn(C5CC5)c4)C3)nc(C3CCn4ccnc4C3)c2nc1C] |
| I-974 | 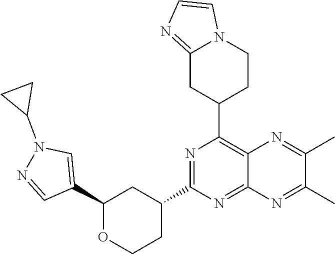 [SMILES: Cc1nc2nc([C@@H]3CCO[C@@H](c4cnn(C5CC5)c4)C3)nc(C3CCn4ccnc4C3)c2nc1C] |
| I-975 | 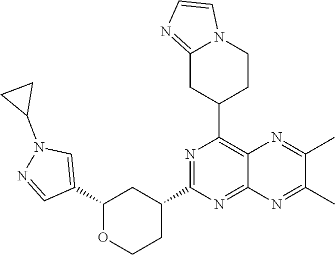 [SMILES: Cc1nc2nc([C@@H]3CCO[C@H](c4cnn(C5CC5)c4)C3)nc(C3CCn4ccnc4C3)c2nc1C] |
| I-976 | 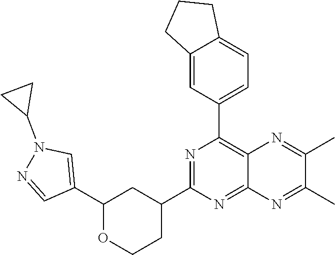 [SMILES: Cc1nc2nc(C3CCOC(c4cnn(C5CC5)c4)C3)nc(-c3ccc4c(c3)CCC4)c2nc1C] |
| I-977 | 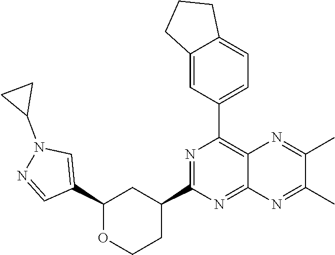 [SMILES: Cc1nc2nc([C@H]3CCO[C@@H](c4cnn(C5CC5)c4)C3)nc(-c3ccc4c(c3)CCC4)c2nc1C] |
| I-978 | 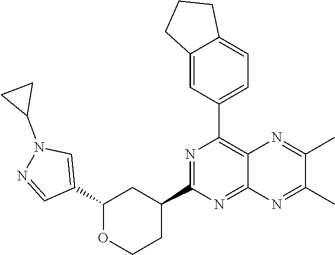 [SMILES: Cc1nc2nc([C@H]3CCO[C@H](c4cnn(C5CC5)c4)C3)nc(-c3ccc4c(c3)CCC4)c2nc1C] |
| I-979 | 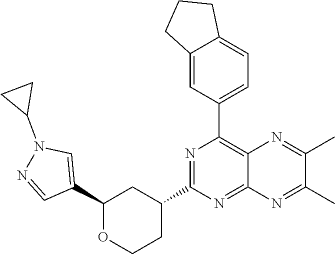 [SMILES: Cc1nc2nc([C@@H]3CCO[C@@H](c4cnn(C5CC5)c4)C3)nc(-c3ccc4c(c3)CCC4)c2nc1C] |
| I-980 | 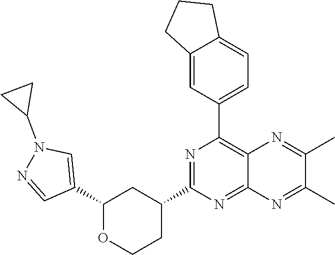 [SMILES: Cc1nc2nc([C@@H]3CCO[C@H](c4cnn(C5CC5)c4)C3)nc(-c3ccc4c(c3)CCC4)c2nc1C] |
| I-981 | 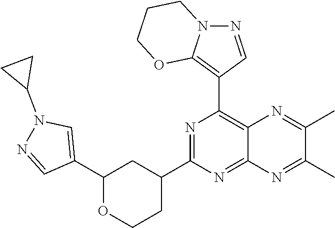 [SMILES: Cc1nc2nc(C3CCOC(c4cnn(C5CC5)c4)C3)nc(-c3cnn4c3OCCC4)c2nc1C] |
| I-982 | 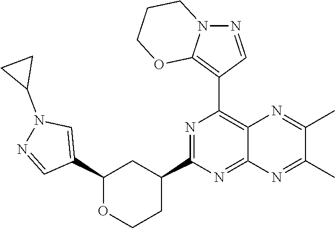 [SMILES: Cc1nc2nc([C@H]3CCO[C@@H](c4cnn(C5CC5)c4)C3)nc(-c3cnn4c3OCCC4)c2nc1C] |
| I-983 | 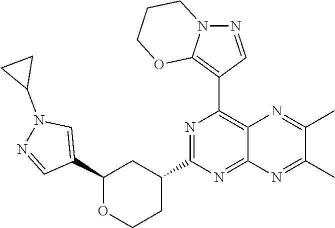 [SMILES: Cc1nc2nc([C@@H]3CCO[C@@H](c4cnn(C5CC5)c4)C3)nc(-c3cnn4c3OCCC4)c2nc1C] |
| I-984 | 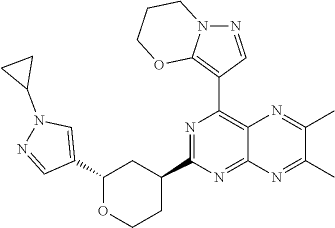 [SMILES: Cc1nc2nc([C@H]3CCO[C@H](c4cnn(C5CC5)c4)C3)nc(-c3cnn4c3OCCC4)c2nc1C] |
| I-985 | 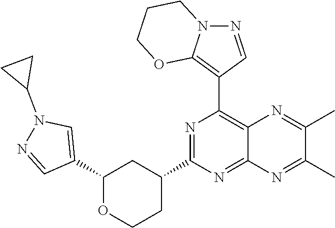 [SMILES: Cc1nc2nc([C@@H]3CCO[C@H](c4cnn(C5CC5)c4)C3)nc(-c3cnn4c3OCCC4)c2nc1C] |
| I-986 | 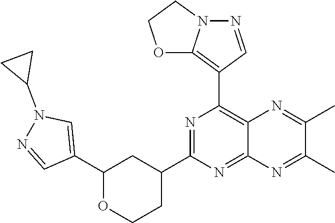 [SMILES: Cc1nc2nc(C3CCOC(c4cnn(C5CC5)c4)C3)nc(-c3cnn4c3OCC4)c2nc1C] |
| I-987 | 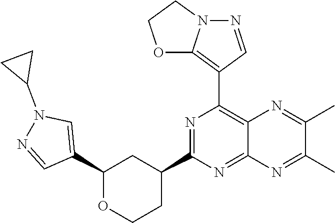 [SMILES: Cc1nc2nc([C@H]3CCO[C@@H](c4cnn(C5CC5)c4)C3)nc(-c3cnn4c3OCC4)c2nc1C] |
| I-988 | 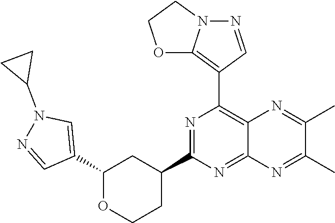 [SMILES: Cc1nc2nc([C@H]3CCO[C@H](c4cnn(C5CC5)c4)C3)nc(-c3cnn4c3OCC4)c2nc1C] |
| I-989 | 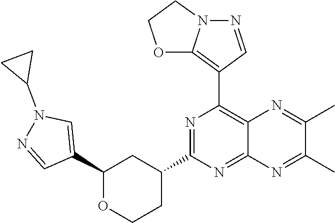 [SMILES: Cc1nc2nc([C@@H]3CCO[C@@H](c4cnn(C5CC5)c4)C3)nc(-c3cnn4c3OCC4)c2nc1C] |
| I-990 | 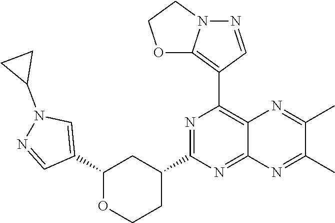 [SMILES: Cc1nc2nc([C@@H]3CCO[C@H](c4cnn(C5CC5)c4)C3)nc(-c3cnn4c3OCC4)c2nc1C] |
| I-991 | 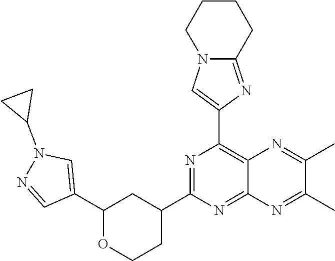 [SMILES: Cc1nc2nc(C3CCOC(c4cnn(C5CC5)c4)C3)nc(-c3cn4c(n3)CCCC4)c2nc1C] |
| I-992 | 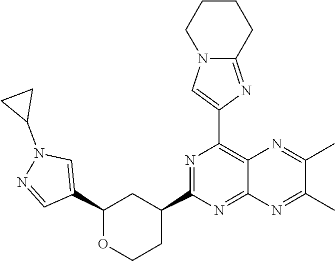 [SMILES: Cc1nc2nc([C@H]3CCO[C@@H](c4cnn(C5CC5)c4)C3)nc(-c3cn4c(n3)CCCC4)c2nc1C] |
| I-993 | 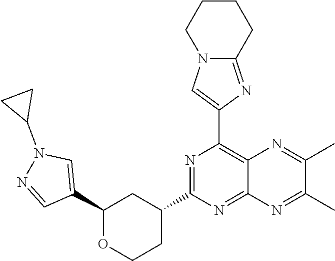 [SMILES: Cc1nc2nc([C@@H]3CCO[C@@H](c4cnn(C5CC5)c4)C3)nc(-c3cn4c(n3)CCCC4)c2nc1C] |
| I-994 | 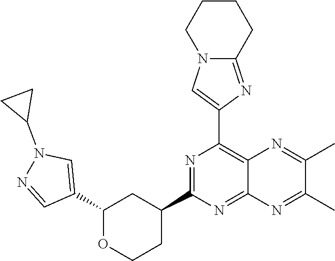 [SMILES: Cc1nc2nc([C@H]3CCO[C@H](c4cnn(C5CC5)c4)C3)nc(-c3cn4c(n3)CCCC4)c2nc1C] |
| I-995 | 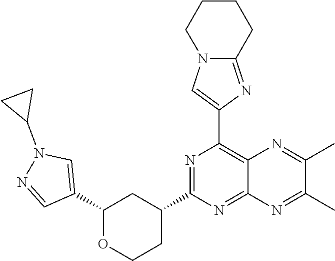 [SMILES: Cc1nc2nc([C@@H]3CCO[C@H](c4cnn(C5CC5)c4)C3)nc(-c3cn4c(n3)CCCC4)c2nc1C] |
| I-996 | 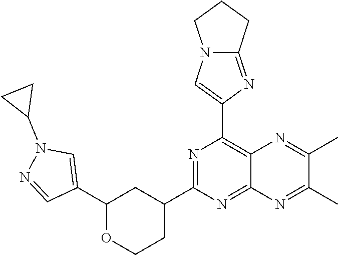 [SMILES: Cc1nc2nc(C3CCOC(c4cnn(C5CC5)c4)C3)nc(-c3cn4c(n3)CCC4)c2nc1C] |
| I-997 | 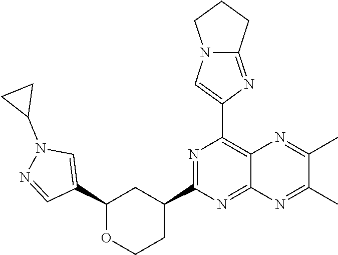 [SMILES: Cc1nc2nc([C@H]3CCO[C@@H](c4cnn(C5CC5)c4)C3)nc(-c3cn4c(n3)CCC4)c2nc1C] |
| I-998 | 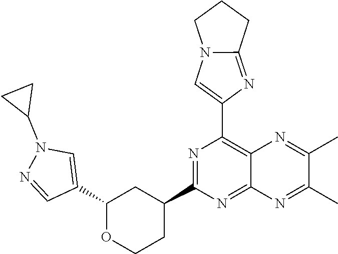 [SMILES: Cc1nc2nc([C@H]3CCO[C@H](c4cnn(C5CC5)c4)C3)nc(-c3cn4c(n3)CCC4)c2nc1C] |
| I-999 | 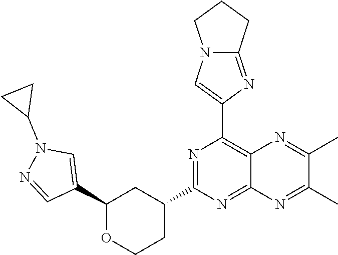 [SMILES: Cc1nc2nc([C@@H]3CCO[C@@H](c4cnn(C5CC5)c4)C3)nc(-c3cn4c(n3)CCC4)c2nc1C] |
| I-1000 | 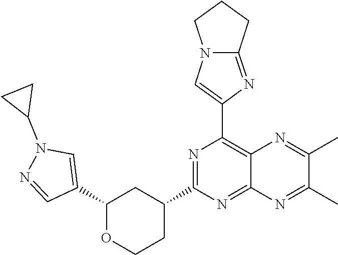 [SMILES: Cc1nc2nc([C@@H]3CCO[C@H](c4cnn(C5CC5)c4)C3)nc(-c3cn4c(n3)CCC4)c2nc1C] |
| I-1001 | 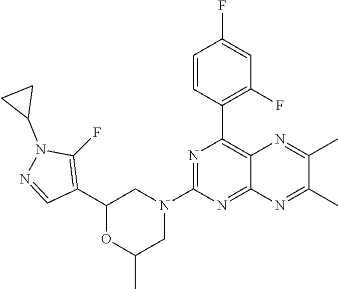 [SMILES: Cc1nc2nc(N3CC(C)OC(c4cnn(C5CC5)c4F)C3)nc(-c3ccc(F)cc3F)c2nc1C] |
| I-1002 | 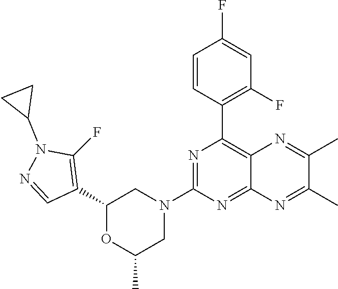 [SMILES: Cc1nc2nc(N3C[C@@H](c4cnn(C5CC5)c4F)O[C@@H](C)C3)nc(-c3ccc(F)cc3F)c2nc1C] |
| I-1003 | 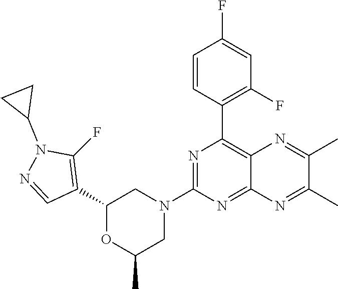 [SMILES: Cc1nc2nc(N3C[C@@H](C)O[C@H](c4cnn(C5CC5)c4F)C3)nc(-c3ccc(F)cc3F)c2nc1C] |
| I-1004 | 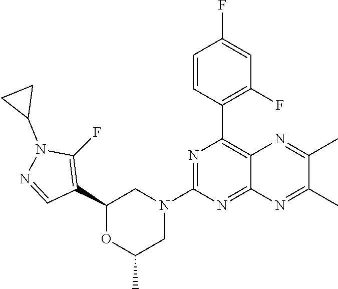 [SMILES: Cc1nc2nc(N3C[C@H](C)O[C@@H](c4cnn(C5CC5)c4F)C3)nc(-c3ccc(F)cc3F)c2nc1C] |
| I-1005 | 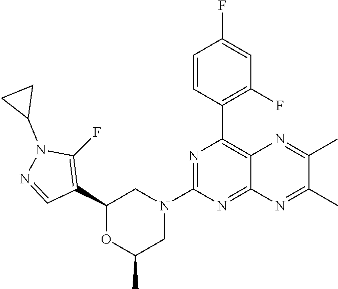 [SMILES: Cc1nc2nc(N3C[C@@H](C)O[C@@H](c4cnn(C5CC5)c4F)C3)nc(-c3ccc(F)cc3F)c2nc1C] |
| I-1006 | 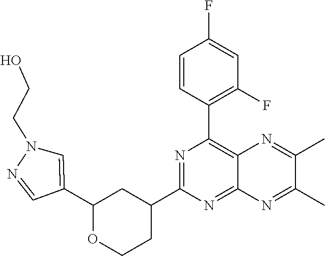 [SMILES: Cc1nc2nc(C3CCOC(c4cnn(CCO)c4)C3)nc(-c3ccc(F)cc3F)c2nc1C] |
| I-1007 | 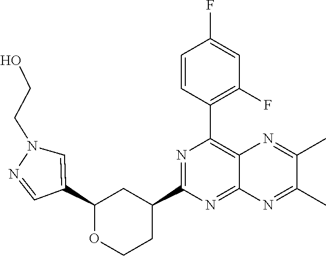 [SMILES: Cc1nc2nc([C@H]3CCO[C@@H](c4cnn(CCO)c4)C3)nc(-c3ccc(F)cc3F)c2nc1C] |
| I-1008 | 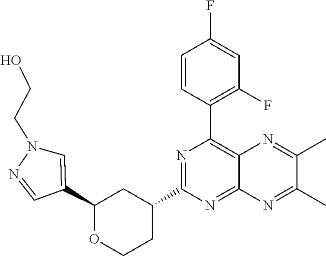 [SMILES: Cc1nc2nc([C@@H]3CCO[C@@H](c4cnn(CCO)c4)C3)nc(-c3ccc(F)cc3F)c2nc1C] |
| I-1009 | 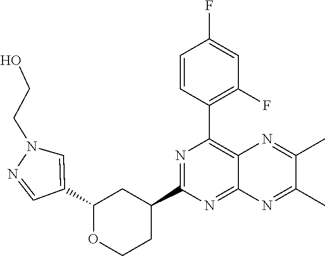 [SMILES: Cc1nc2nc([C@H]3CCO[C@H](c4cnn(CCO)c4)C3)nc(-c3ccc(F)cc3F)c2nc1C] |
| I-1010 | 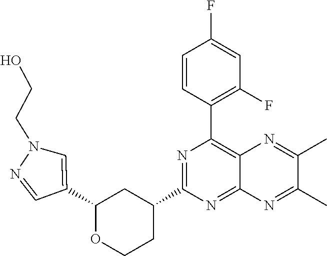 [SMILES: Cc1nc2nc([C@@H]3CCO[C@H](c4cnn(CCO)c4)C3)nc(-c3ccc(F)cc3F)c2nc1C] |
| I-1011 | 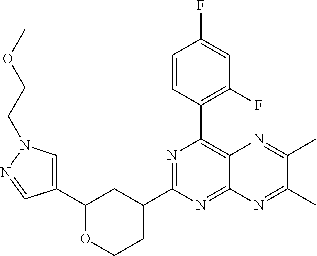 [SMILES: COCCn1cc(C2CC(c3nc(-c4ccc(F)cc4F)c4nc(C)c(C)nc4n3)CCO2)cn1] |
| I-1012 | 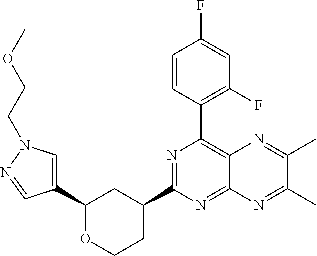 [SMILES: COCCn1cc([C@H]2C[C@@H](c3nc(-c4ccc(F)cc4F)c4nc(C)c(C)nc4n3)CCO2)cn1] |
| I-1013 | 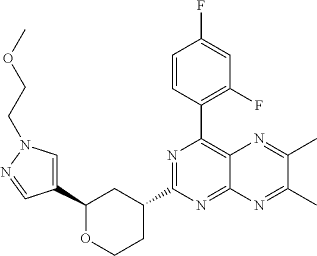 [SMILES: COCCn1cc([C@H]2C[C@H](c3nc(-c4ccc(F)cc4F)c4nc(C)c(C)nc4n3)CCO2)cn1] |
| I-1014 | 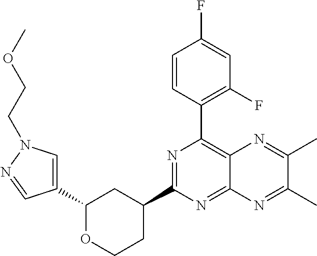 [SMILES: COCCn1cc([C@@H]2C[C@@H](c3nc(-c4ccc(F)cc4F)c4nc(C)c(C)nc4n3)CCO2)cn1] |
| I-1015 | 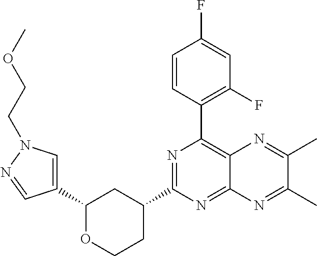 [SMILES: COCCn1cc([C@@H]2C[C@H](c3nc(-c4ccc(F)cc4F)c4nc(C)c(C)nc4n3)CCO2)cn1] |
| I-1016 | 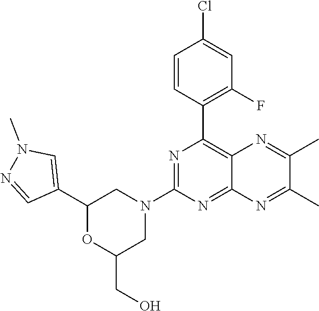 [SMILES: Cc1nc2nc(N3CC(CO)OC(c4cnn(C)c4)C3)nc(-c3ccc(Cl)cc3F)c2nc1C] |
| I-1017 | 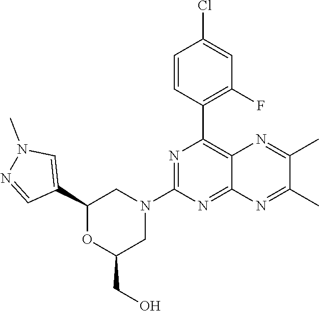 [SMILES: Cc1nc2nc(N3C[C@@H](CO)O[C@@H](c4cnn(C)c4)C3)nc(-c3ccc(Cl)cc3F)c2nc1C] |
| I-1018 | 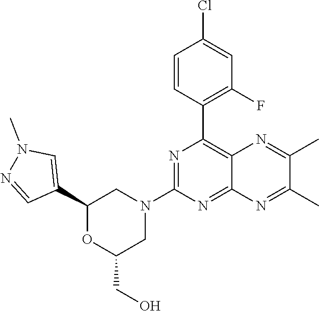 [SMILES: Cc1nc2nc(N3C[C@H](CO)O[C@@H](c4cnn(C)c4)C3)nc(-c3ccc(Cl)cc3F)c2nc1C] |
| I-1019 | 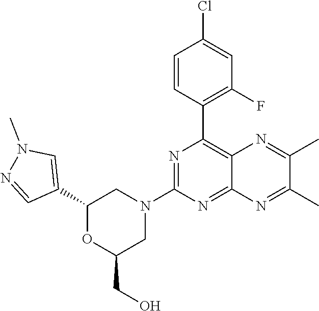 [SMILES: Cc1nc2nc(N3C[C@@H](CO)O[C@H](c4cnn(C)c4)C3)nc(-c3ccc(Cl)cc3F)c2nc1C] |
| I-1020 | 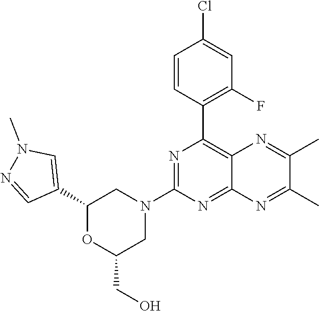 [SMILES: Cc1nc2nc(N3C[C@H](CO)O[C@H](c4cnn(C)c4)C3)nc(-c3ccc(Cl)cc3F)c2nc1C] |
| I-1021 | 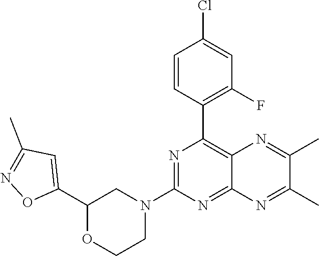 [SMILES: Cc1cc(C2CN(c3nc(-c4ccc(Cl)cc4F)c4nc(C)c(C)nc4n3)CCO2)on1] |
| I-1022 | 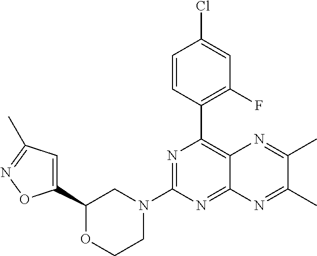 [SMILES: Cc1cc([C@H]2CN(c3nc(-c4ccc(Cl)cc4F)c4nc(C)c(C)nc4n3)CCO2)on1] |
| I-1023 | 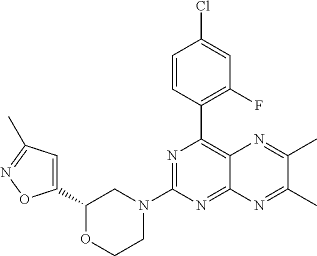 [SMILES: Cc1cc([C@@H]2CN(c3nc(-c4ccc(Cl)cc4F)c4nc(C)c(C)nc4n3)CCO2)on1] |
| I-1024 | 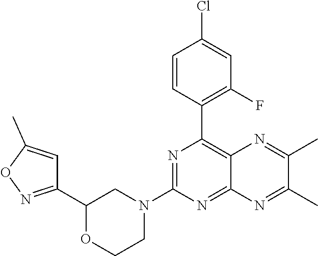 [SMILES: Cc1cc(C2CN(c3nc(-c4ccc(Cl)cc4F)c4nc(C)c(C)nc4n3)CCO2)no1] |
| I-1025 | 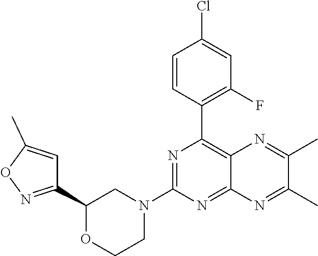 [SMILES: Cc1cc([C@H]2CN(c3nc(-c4ccc(Cl)cc4F)c4nc(C)c(C)nc4n3)CCO2)no1] |
| I-1026 | 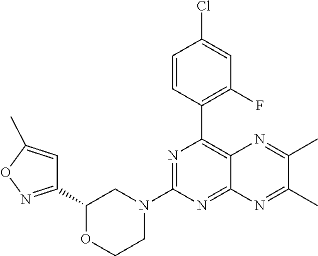 [SMILES: Cc1cc([C@@H]2CN(c3nc(-c4ccc(Cl)cc4F)c4nc(C)c(C)nc4n3)CCO2)no1] |
| I-1027 | 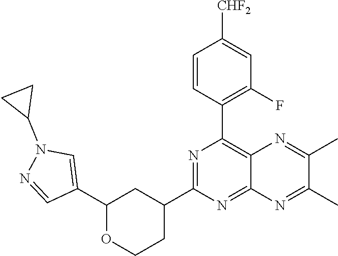 [SMILES: Cc1nc2nc(C3CCOC(c4cnn(C5CC5)c4)C3)nc(-c3ccc(C(F)F)cc3F)c2nc1C] |
| I-1028 | 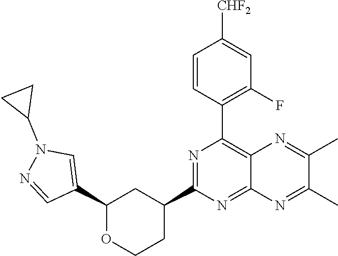 [SMILES: Cc1nc2nc([C@H]3CCO[C@@H](c4cnn(C5CC5)c4)C3)nc(-c3ccc(C(F)F)cc3F)c2nc1C] |
| I-1029 | 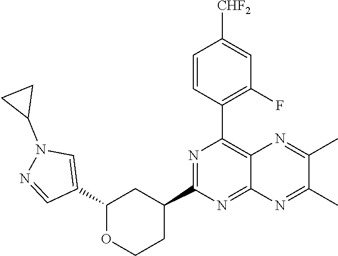 [SMILES: Cc1nc2nc([C@H]3CCO[C@H](c4cnn(C5CC5)c4)C3)nc(-c3ccc(C(F)F)cc3F)c2nc1C] |
| I-1030 | 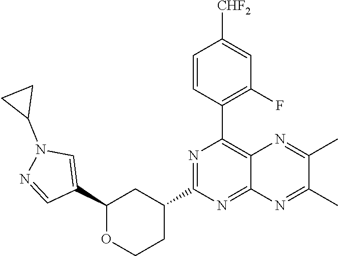 [SMILES: Cc1nc2nc([C@@H]3CCO[C@@H](c4cnn(C5CC5)c4)C3)nc(-c3ccc(C(F)F)cc3F)c2nc1C] |
| I-1031 | 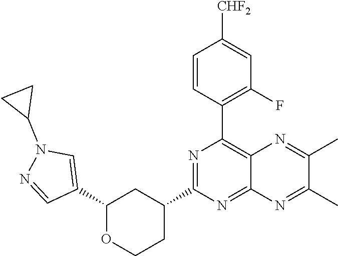 [SMILES: Cc1nc2nc([C@@H]3CCO[C@H](c4cnn(C5CC5)c4)C3)nc(-c3ccc(C(F)F)cc3F)c2nc1C] |
| I-1032 | 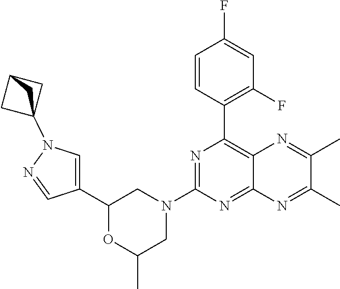 |
| I-1033 | 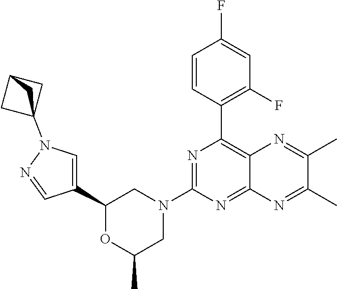 |
| I-1034 | 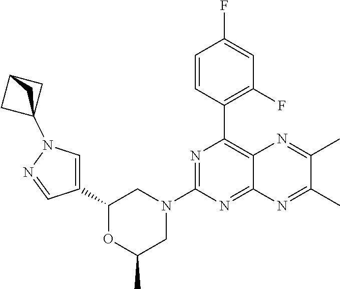 |
| I-1035 | 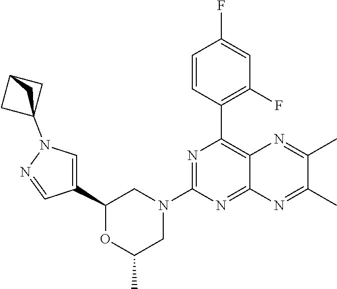 |
| I-1036 | 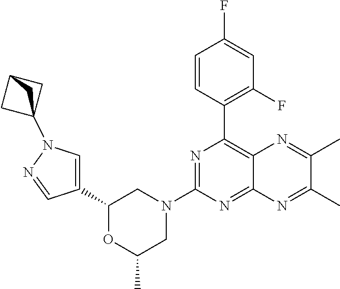 |
| I-1037 | 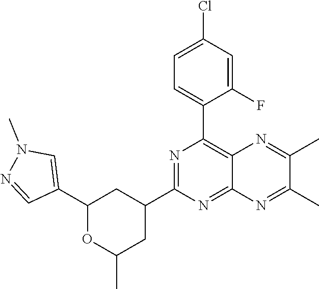 [SMILES: Cc1nc2nc(C3CC(C)OC(c4cnn(C)c4)C3)nc(-c3ccc(Cl)cc3F)c2nc1C] |
| I-1038 | 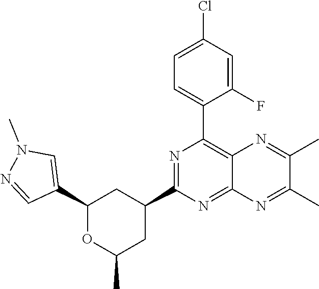 [SMILES: Cc1nc2nc([C@H]3C[C@@H](C)O[C@@H](c4cnn(C)c4)C3)nc(-c3ccc(Cl)cc3F)c2nc1C] |
| I-1039 | 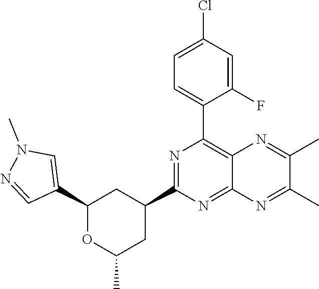 [SMILES: Cc1nc2nc([C@H]3C[C@H](C)O[C@@H](c4cnn(C)c4)C3)nc(-c3ccc(Cl)cc3F)c2nc1C] |
| I-1040 | 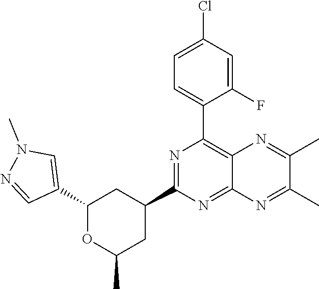 [SMILES: Cc1nc2nc([C@H]3C[C@@H](C)O[C@H](c4cnn(C)c4)C3)nc(-c3ccc(Cl)cc3F)c2nc1C] |
| I-1041 | 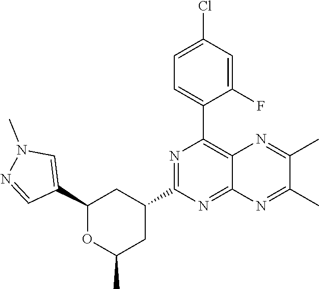 [SMILES: Cc1nc2nc([C@@H]3C[C@@H](C)O[C@@H](c4cnn(C)c4)C3)nc(-c3ccc(Cl)cc3F)c2nc1C] |
| I-1042 | 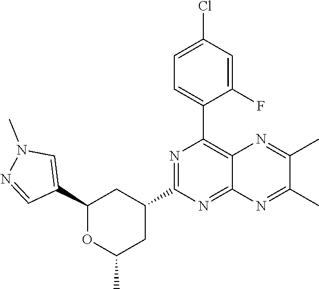 [SMILES: Cc1nc2nc([C@@H]3C[C@H](C)O[C@@H](c4cnn(C)c4)C3)nc(-c3ccc(Cl)cc3F)c2nc1C] |
| I-1043 | 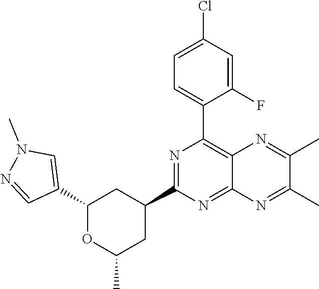 [SMILES: Cc1nc2nc([C@@H]3C[C@@H](c4cnn(C)c4)O[C@@H](C)C3)nc(-c3ccc(Cl)cc3F)c2nc1C] |
| I-1044 | 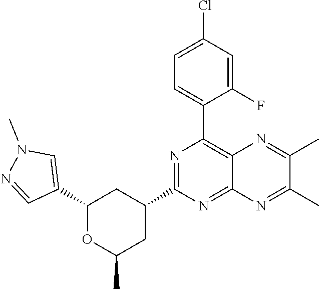 [SMILES: Cc1nc2nc([C@@H]3C[C@@H](C)O[C@H](c4cnn(C)c4)C3)nc(-c3ccc(Cl)cc3F)c2nc1C] |
| I-1045 | 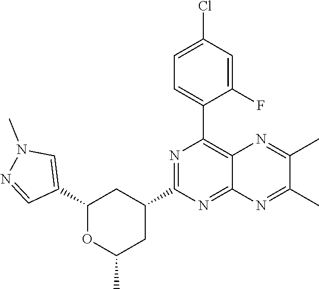 [SMILES: Cc1nc2nc([C@H]3C[C@@H](c4cnn(C)c4)O[C@@H](C)C3)nc(-c3ccc(Cl)cc3F)c2nc1C] |
| I-1046 | 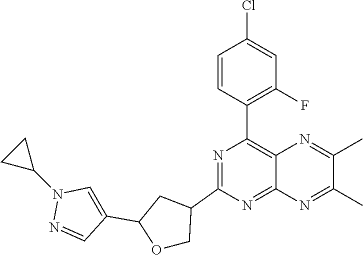 [SMILES: Cc1nc2nc(C3COC(c4cnn(C5CC5)c4)C3)nc(-c3ccc(Cl)cc3F)c2nc1C] |
| I-1047 | 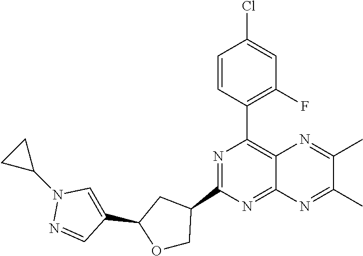 [SMILES: Cc1nc2nc([C@H]3CO[C@@H](c4cnn(C5CC5)c4)C3)nc(-c3ccc(Cl)cc3F)c2nc1C] |
| I-1048 | 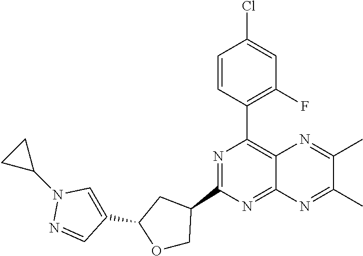 [SMILES: Cc1nc2nc([C@H]3CO[C@H](c4cnn(C5CC5)c4)C3)nc(-c3ccc(Cl)cc3F)c2nc1C] |
| I-1049 | 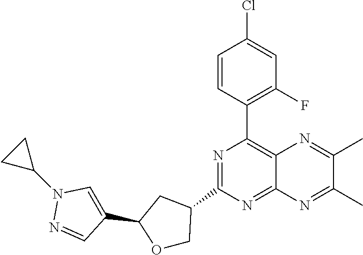 [SMILES: Cc1nc2nc([C@@H]3CO[C@@H](c4cnn(C5CC5)c4)C3)nc(-c3ccc(Cl)cc3F)c2nc1C] |
| I-1050 | 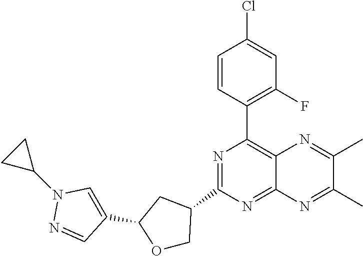 [SMILES: Cc1nc2nc([C@@H]3CO[C@H](c4cnn(C5CC5)c4)C3)nc(-c3ccc(Cl)cc3F)c2nc1C] |
| I-1051 | 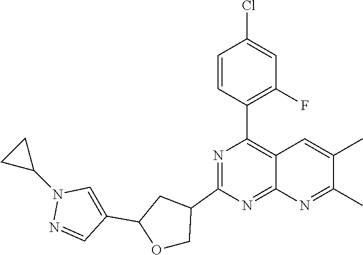 [SMILES: Cc1cc2c(-c3ccc(Cl)cc3F)nc(C3COC(c4cnn(C5CC5)c4)C3)nc2nc1C] |
| I-1052 | 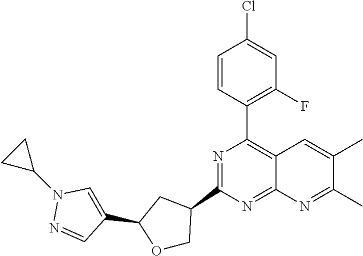 [SMILES: Cc1cc2c(-c3ccc(Cl)cc3F)nc([C@H]3CO[C@@H](c4cnn(C5CC5)c4)C3)nc2nc1C] |
| I-1053 | 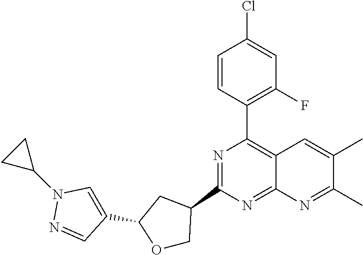 [SMILES: Cc1cc2c(-c3ccc(Cl)cc3F)nc([C@H]3CO[C@H](c4cnn(C5CC5)c4)C3)nc2nc1C] |
| I-1054 | 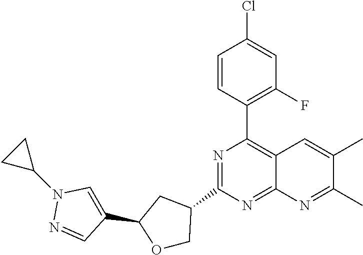 [SMILES: Cc1cc2c(-c3ccc(Cl)cc3F)nc([C@@H]3CO[C@@H](c4cnn(C5CC5)c4)C3)nc2nc1C] |
| I-1055 | 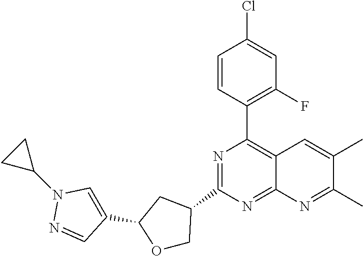 [SMILES: Cc1cc2c(-c3ccc(Cl)cc3F)nc([C@@H]3CO[C@H](c4cnn(C5CC5)c4)C3)nc2nc1C] |
| I-1056 | 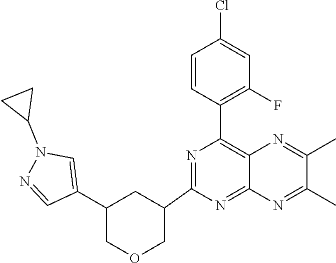 [SMILES: Cc1nc2nc(C3COCC(c4cnn(C5CC5)c4)C3)nc(-c3ccc(Cl)cc3F)c2nc1C] |
| I-1057 | 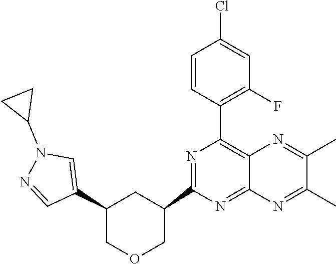 [SMILES: Cc1nc2nc([C@H]3COC[C@@H](c4cnn(C5CC5)c4)C3)nc(-c3ccc(Cl)cc3F)c2nc1C] |
| I-1058 | 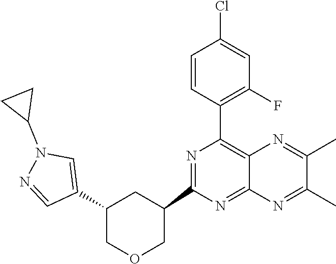 [SMILES: Cc1nc2nc([C@H]3COC[C@H](c4cnn(C5CC5)c4)C3)nc(-c3ccc(Cl)cc3F)c2nc1C] |
| I-1059 | 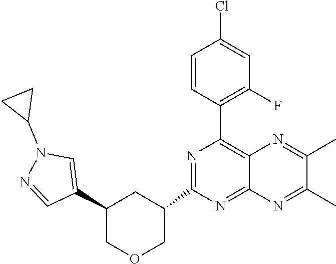 [SMILES: Cc1nc2nc([C@@H]3COC[C@@H](c4cnn(C5CC5)c4)C3)nc(-c3ccc(Cl)cc3F)c2nc1C] |
| I-1060 | 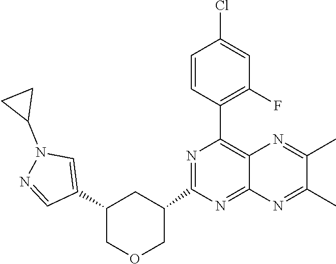 [SMILES: Cc1nc2nc([C@@H]3COC[C@H](c4cnn(C5CC5)c4)C3)nc(-c3ccc(Cl)cc3F)c2nc1C] |
| I-1061 | 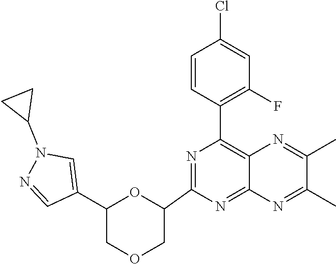 [SMILES: Cc1nc2nc(C3COCC(c4cnn(C5CC5)c4)O3)nc(-c3ccc(Cl)cc3F)c2nc1C] |
| I-1062 | 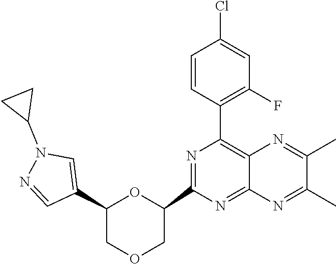 [SMILES: Cc1nc2nc([C@H]3COC[C@@H](c4cnn(C5CC5)c4)O3)nc(-c3ccc(Cl)cc3F)c2nc1C] |
| I-1063 | 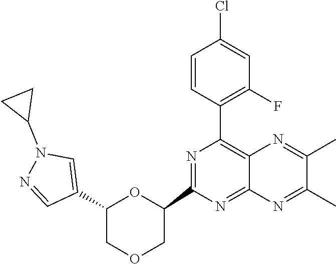 [SMILES: Cc1nc2nc([C@H]3COC[C@H](c4cnn(C5CC5)c4)O3)nc(-c3ccc(Cl)cc3F)c2nc1C] |
| I-1064 | 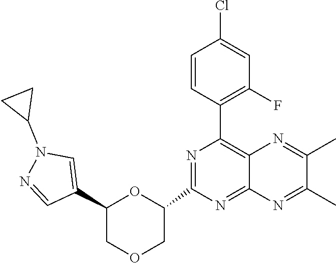 [SMILES: Cc1nc2nc([C@@H]3COC[C@@H](c4cnn(C5CC5)c4)O3)nc(-c3ccc(Cl)cc3F)c2nc1C] |
| I-1065 | 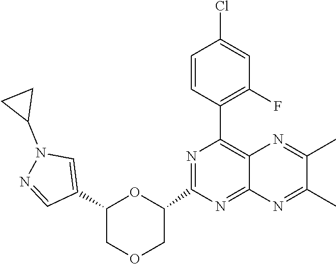 [SMILES: Cc1nc2nc([C@@H]3COC[C@H](c4cnn(C5CC5)c4)O3)nc(-c3ccc(Cl)cc3F)c2nc1C] |
| I-1066 | 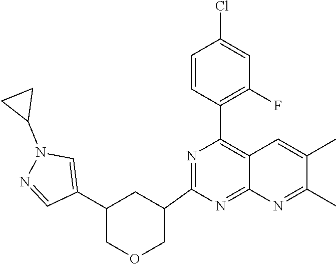 [SMILES: Cc1cc2c(-c3ccc(Cl)cc3F)nc(C3COCC(c4cnn(C5CC5)c4)C3)nc2nc1C] |
| I-1067 | 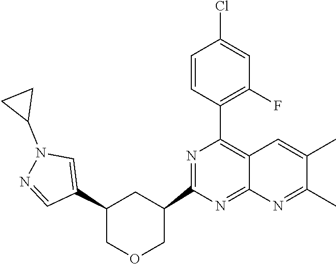 [SMILES: Cc1cc2c(-c3ccc(Cl)cc3F)nc([C@H]3COC[C@@H](c4cnn(C5CC5)c4)C3)nc2nc1C] |
| I-1068 | 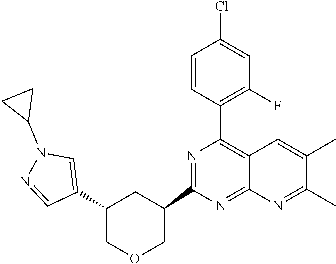 [SMILES: Cc1cc2c(-c3ccc(Cl)cc3F)nc([C@H]3COC[C@H](c4cnn(C5CC5)c4)C3)nc2nc1C] |
| I-1069 | 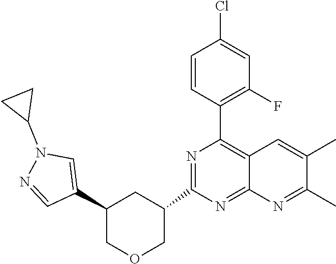 [SMILES: Cc1cc2c(-c3ccc(Cl)cc3F)nc([C@@H]3COC[C@@H](c4cnn(C5CC5)c4)C3)nc2nc1C] |
| I-1070 | 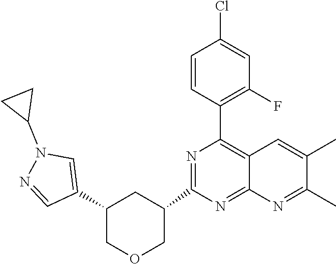 [SMILES: Cc1cc2c(-c3ccc(Cl)cc3F)nc([C@@H]3COC[C@H](c4cnn(C5CC5)c4)C3)nc2nc1C] |

In some embodiments, exemplary compounds of the invention are set forth in Table A-2, below. In some embodiments, the compound is a compound set forth in Table A-2, or a pharmaceutically acceptable salt thereof.

TABLE A-2
Exemplary Compounds

| | |
|---|---|
| 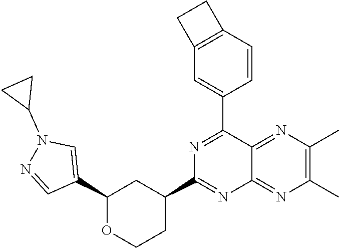 [SMILES: Cc1nc2nc([C@H]3CCO[C@@H](c4cnn(C5CC5)c4)C3)nc(-c3ccc4c(c3)CC4)c2nc1C] | I-1071 |
| 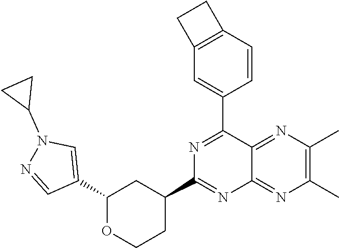 [SMILES: Cc1nc2nc([C@H]3CCO[C@H](c4cnn(C5CC5)c4)C3)nc(-c3ccc4c(c3)CC4)c2nc1C] | I-1072 |
| 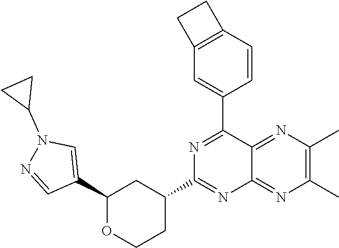 [SMILES: Cc1nc2nc([C@@H]3CCO[C@@H](c4cnn(C5CC5)c4)C3)nc(-c3ccc4c(c3)CC4)c2nc1C] | I-1073 |
| 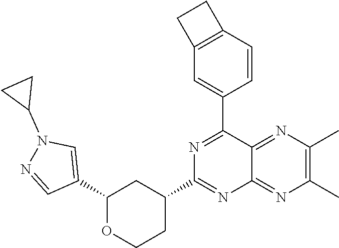 [SMILES: Cc1nc2nc([C@@H]3CCO[C@H](c4cnn(C5CC5)c4)C3)nc(-c3ccc4c(c3)CC4)c2nc1C] | I-1074 |
| 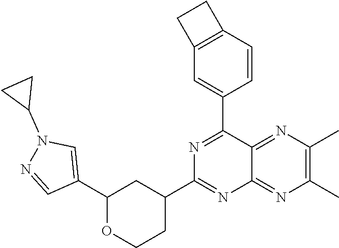 [SMILES: Cc1nc2nc(C3CCOC(c4cnn(C5CC5)c4)C3)nc(-c3ccc4c(c3)CC4)c2nc1C] | I-1075 |
| 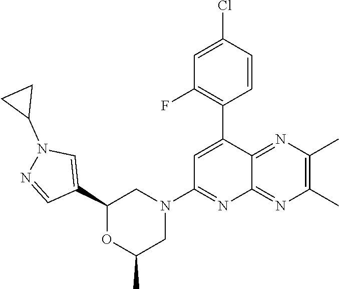 [SMILES: Cc1nc2nc(N3C[C@@H](C)O[C@@H](c4cnn(C5CC5)c4)C3)cc(-c3ccc(Cl)cc3F)c2nc1C] | I-1076 |
| 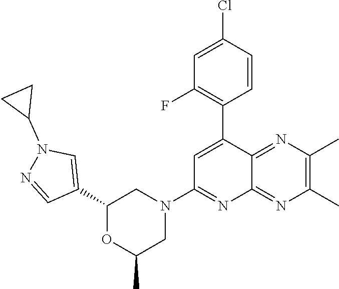 [SMILES: Cc1nc2nc(N3C[C@@H](C)O[C@H](c4cnn(C5CC5)c4)C3)cc(-c3ccc(Cl)cc3F)c2nc1C] | I-1077 |
| 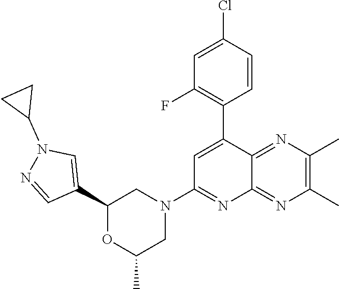 [SMILES: Cc1nc2nc(N3C[C@H](C)O[C@@H](c4cnn(C5CC5)c4)C3)cc(-c3ccc(Cl)cc3F)c2nc1C] | I-1078 |
| 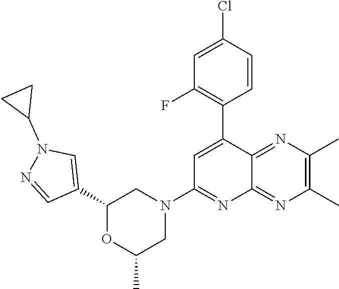 [SMILES: Cc1nc2nc(N3C[C@@H](c4cnn(C5CC5)c4)O[C@@H](C)C3)cc(-c3ccc(Cl)cc3F)c2nc1C] | I-1079 |
| 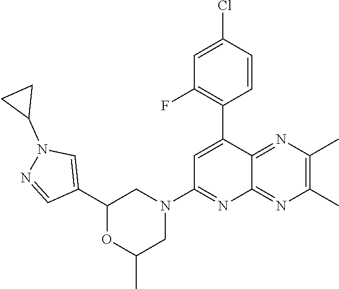 [SMILES: Cc1nc2nc(N3CC(C)OC(c4cnn(C5CC5)c4)C3)cc(-c3ccc(Cl)cc3F)c2nc1C] | I-1080 |
| 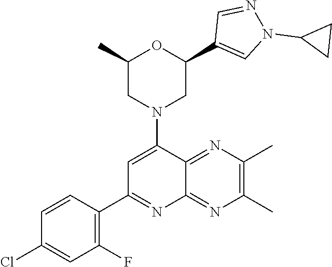 [SMILES: Cc1nc2nc(-c3ccc(Cl)cc3F)cc(N3C[C@@H](C)O[C@@H](c4cnn(C5CC5)c4)C3)c2nc1C] | I-1081 |
| 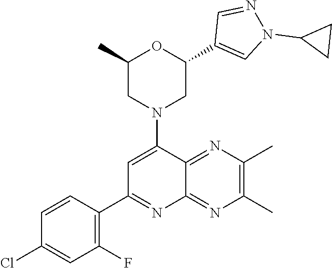 [SMILES: Cc1nc2nc(-c3ccc(Cl)cc3F)cc(N3C[C@@H](C)O[C@H](c4cnn(C5CC5)c4)C3)c2nc1C] | I-1082 |
| 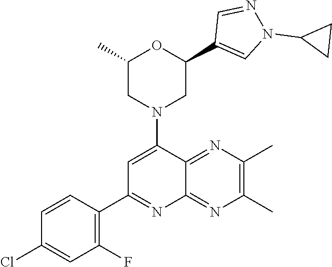 | I-1083 |
| 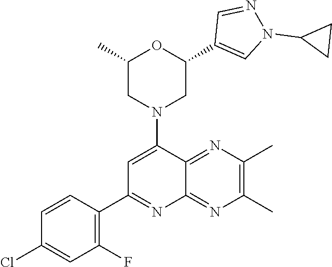 [SMILES: Cc1nc2nc(-c3ccc(Cl)cc3F)cc(N3C[C@@H](c4cnn(C5CC5)c4)O[C@@H](C)C3)c2nc1C] | I-1084 |
| 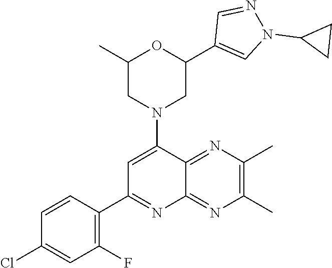 [SMILES: Cc1nc2nc(-c3ccc(Cl)cc3F)cc(N3CC(C)OC(c4cnn(C5CC5)c4)C3)c2nc1C] | I-1085 |
| 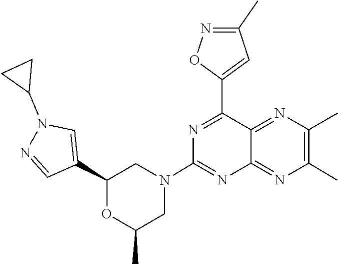 [SMILES: Cc1cc(-c2nc(N3C[C@@H](C)O[C@@H](c4cnn(C5CC5)c4)C3)nc3nc(C)c(C)nc23)on1] | I-1086 |
| 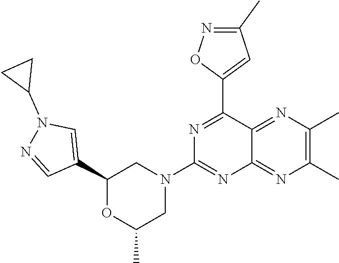 [SMILES: Cc1cc(-c2nc(N3C[C@H](C)O[C@@H](c4cnn(C5CC5)c4)C3)nc3nc(C)c(C)nc23)on1] | I-1087 |
| 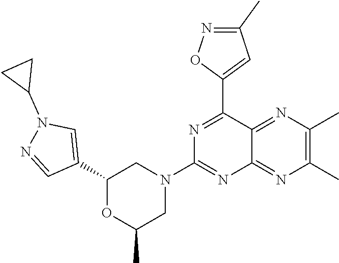 [SMILES: Cc1cc(-c2nc(N3C[C@@H](C)O[C@H](c4cnn(C5CC5)c4)C3)nc3nc(C)c(C)nc23)on1] | I-1088 |
| 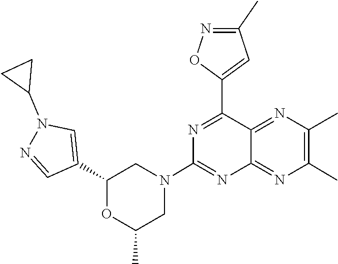 [SMILES: Cc1cc(-c2nc(N3C[C@@H](c4cnn(C5CC5)c4)O[C@@H](C)C3)nc3nc(C)c(C)nc23)on1] | I-1089 |
| 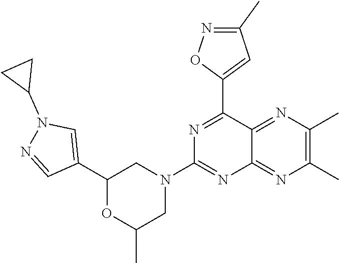 [SMILES: Cc1cc(-c2nc(N3CC(C)OC(c4cnn(C5CC5)c4)C3)nc3nc(C)c(C)nc23)on1] | I-1090 |
| 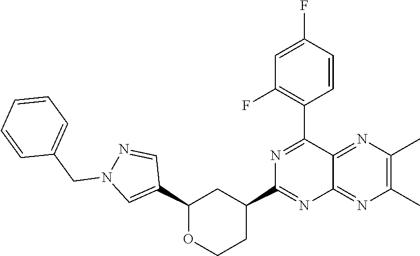 [SMILES: Cc1nc2nc([C@H]3CCO[C@@H](c4cnn(Cc5ccccc5)c4)C3)nc(-c3ccc(F)cc3F)c2nc1C] | I-1091 |
| 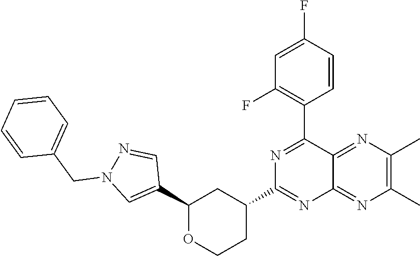 [SMILES: Cc1nc2nc([C@@H]3CCO[C@@H](c4cnn(Cc5ccccc5)c4)C3)nc(-c3ccc(F)cc3F)c2nc1C] | I-1092 |
| 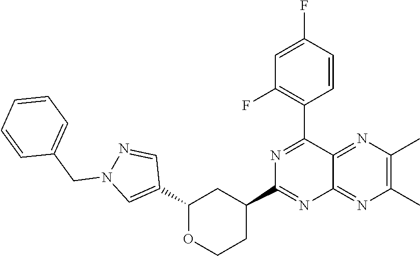 [SMILES: Cc1nc2nc([C@H]3CCO[C@H](c4cnn(Cc5ccccc5)c4)C3)nc(-c3ccc(F)cc3F)c2nc1C] | I-1093 |
| 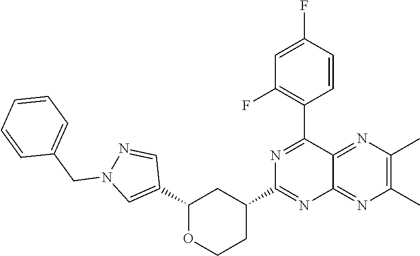 [SMILES: Cc1nc2nc([C@@H]3CCO[C@H](c4cnn(Cc5ccccc5)c4)C3)nc(-c3ccc(F)cc3F)c2nc1C] | I-1094 |
| 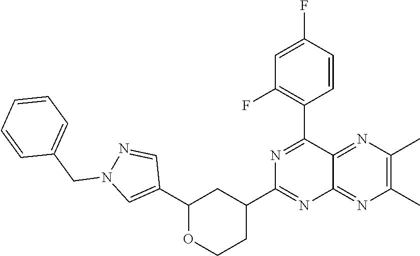 [SMILES: Cc1nc2nc(C3CCOC(c4cnn(Cc5ccccc5)c4)C3)nc(-c3ccc(F)cc3F)c2nc1C] | I-1095 |
| 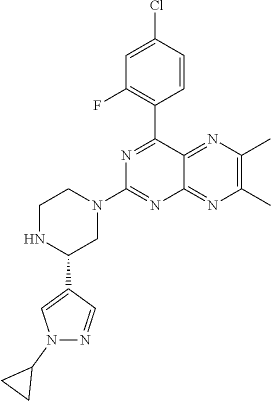 [SMILES: Cc1nc2nc(N3CCN[C@@H](c4cnn(C5CC5)c4)C3)nc(-c3ccc(Cl)cc3F)c2nc1C] | I-1096 |
| 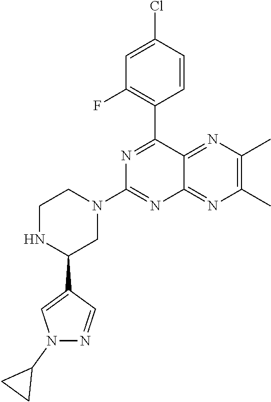 [SMILES: Cc1nc2nc(N3CCN[C@H](c4cnn(C5CC5)c4)C3)nc(-c3ccc(Cl)cc3F)c2nc1C] | I-1097 |
| 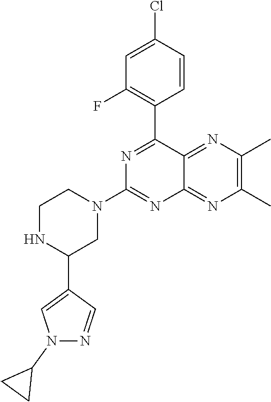 [SMILES: Cc1nc2nc(N3CCNC(c4cnn(C5CC5)c4)C3)nc(-c3ccc(Cl)cc3F)c2nc1C] | I-1098 |
| 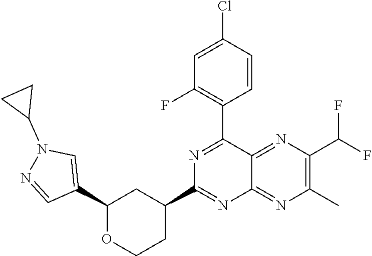 [SMILES: Cc1nc2nc([C@H]3CCO[C@@H](c4cnn(C5CC5)c4)C3)nc(-c3ccc(Cl)cc3F)c2nc1C(F)F] | I-1099 |
| 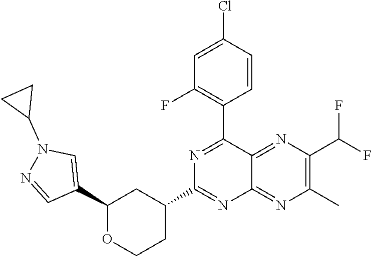 [SMILES: Cc1nc2nc([C@@H]3CCO[C@@H](c4cnn(C5CC5)c4)C3)nc(-c3ccc(Cl)cc3F)c2nc1C(F)F] | I-1100 |
| 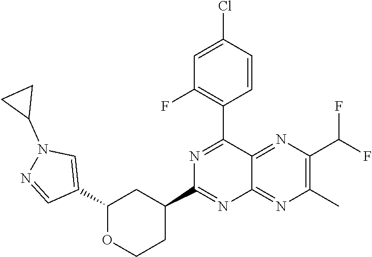 [SMILES: Cc1nc2nc([C@H]3CCO[C@H](c4cnn(C5CC5)c4)C3)nc(-c3ccc(Cl)cc3F)c2nc1C(F)F] | I-1101 |
| 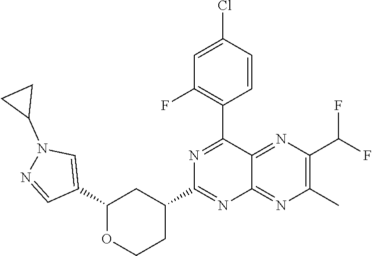 [SMILES: Cc1nc2nc([C@@H]3CCO[C@H](c4cnn(C5CC5)c4)C3)nc(-c3ccc(Cl)cc3F)c2nc1C(F)F] | I-1102 |
| 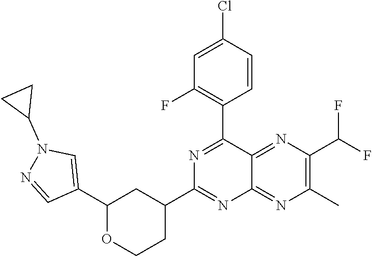 [SMILES: Cc1nc2nc(C3CCOC(c4cnn(C5CC5)c4)C3)nc(-c3ccc(Cl)cc3F)c2nc1C(F)F] | I-1103 |
| 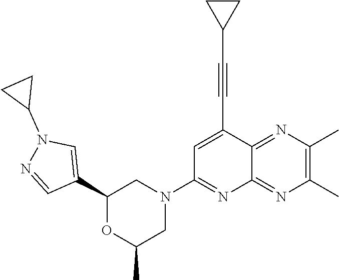 [SMILES: Cc1nc2nc(N3C[C@@H](C)O[C@@H](c4cnn(C5CC5)c4)C3)cc(C#CC3CC3)c2nc1C] | I-1104 |
| 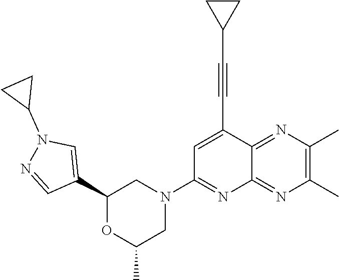 [SMILES: Cc1nc2nc(N3C[C@H](C)O[C@@H](c4cnn(C5CC5)c4)C3)cc(C#CC3CC3)c2nc1C] | I-1105 |
| 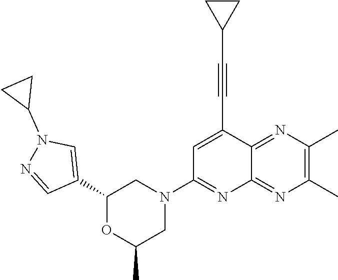 [SMILES: Cc1nc2nc(N3C[C@@H](C)O[C@H](c4cnn(C5CC5)c4)C3)cc(C#CC3CC3)c2nc1C] | I-1106 |
| 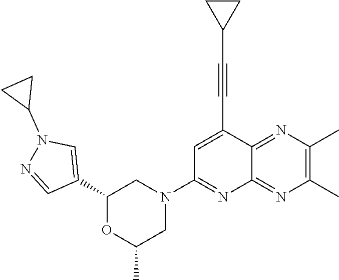 [SMILES: Cc1nc2nc(N3C[C@@H](c4cnn(C5CC5)c4)O[C@@H](C)C3)cc(C#CC3CC3)c2nc1C] | I-1107 |
| 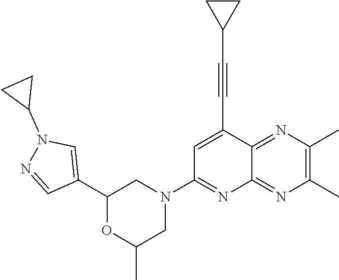 [SMILES: Cc1nc2nc(N3CC(C)OC(c4cnn(C5CC5)c4)C3)cc(C#CC3CC3)c2nc1C] | I-1108 |
| 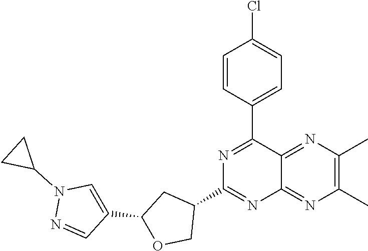 [SMILES: Cc1nc2nc([C@@H]3CO[C@H](c4cnn(C5CC5)c4)C3)nc(-c3ccc(Cl)cc3)c2nc1C] | I-1109 |
| 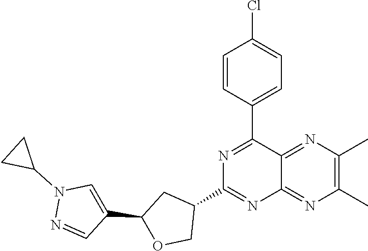 [SMILES: Cc1nc2nc([C@@H]3CO[C@@H](c4cnn(C5CC5)c4)C3)nc(-c3ccc(Cl)cc3)c2nc1C] | I-1110 |
| 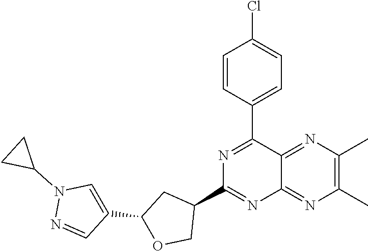 [SMILES: Cc1nc2nc([C@H]3CO[C@H](c4cnn(C5CC5)c4)C3)nc(-c3ccc(Cl)cc3)c2nc1C] | I-1111 |
| 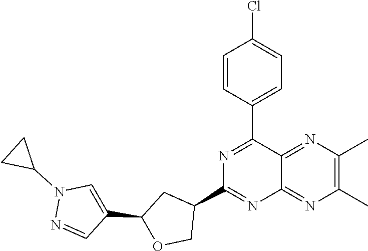 [SMILES: Cc1nc2nc([C@H]3CO[C@@H](c4cnn(C5CC5)c4)C3)nc(-c3ccc(Cl)cc3)c2nc1C] | I-1112 |
| 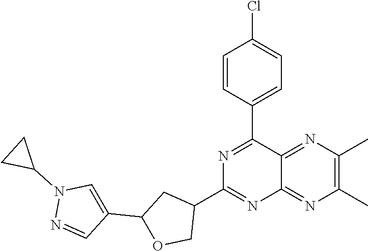 [SMILES: Cc1nc2nc(C3COC(c4cnn(C5CC5)c4)C3)nc(-c3ccc(Cl)cc3)c2nc1C] | I-1113 |
| 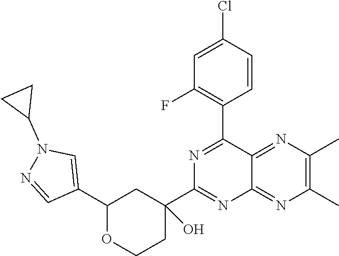 [SMILES: Cc1nc2nc(C3(O)CCOC(c4cnn(C5CC5)c4)C3)nc(-c3ccc(Cl)cc3F)c2nc1C] | I-1114 |
| 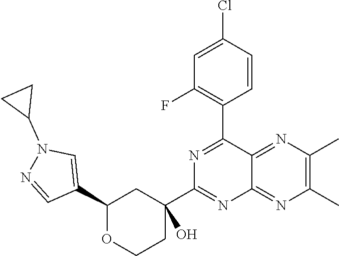 [SMILES: Cc1nc2nc([C@]3(O)CCO[C@@H](c4cnn(C5CC5)c4)C3)nc(-c3ccc(Cl)cc3F)c2nc1C] | I-1115 |
| 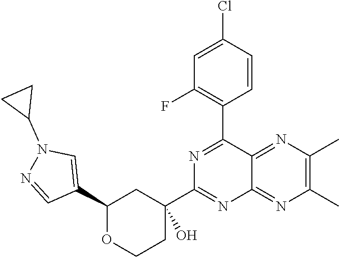 [SMILES: Cc1nc2nc([C@@]3(O)CCO[C@@H](c4cnn(C5CC5)c4)C3)nc(-c3ccc(Cl)cc3F)c2nc1C] | I-1116 |
| 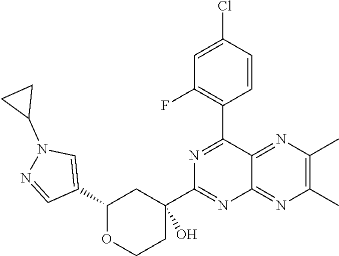 [SMILES: Cc1nc2nc([C@@]3(O)CCO[C@H](c4cnn(C5CC5)c4)C3)nc(-c3ccc(Cl)cc3F)c2nc1C] | I-1117 |
| 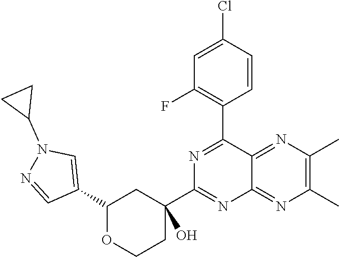 | I-1118 |
| 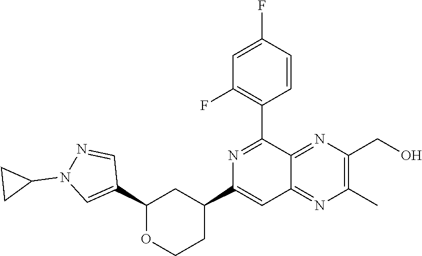 [SMILES: Cc1nc2cc([C@H]3CCO[C@@H](c4cnn(C5CC5)c4)C3)nc(-c3ccc(F)cc3F)c2nc1CO] | I-1119 |
| 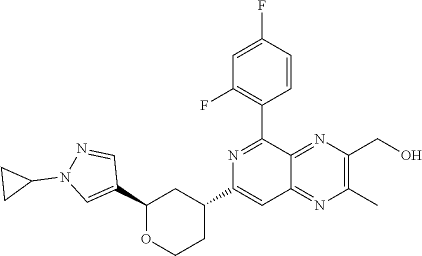 [SMILES: Cc1nc2cc([C@@H]3CCO[C@@H](c4cnn(C5CC5)c4)C3)nc(-c3ccc(F)cc3F)c2nc1CO] | I-1120 |
| 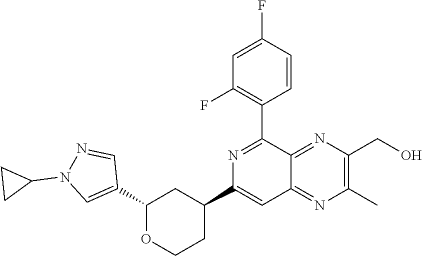 [SMILES: Cc1nc2cc([C@H]3CCO[C@H](c4cnn(C5CC5)c4)C3)nc(-c3ccc(F)cc3F)c2nc1CO] | I-1121 |
| 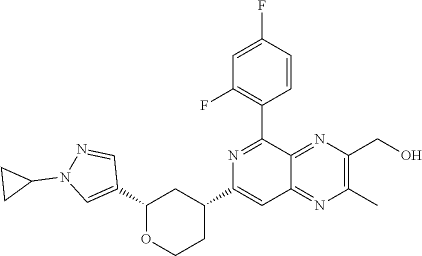 [SMILES: Cc1nc2cc([C@@H]3CCO[C@H](c4cnn(C5CC5)c4)C3)nc(-c3ccc(F)cc3F)c2nc1CO] | I-1122 |
| 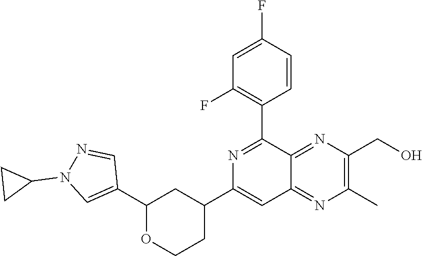 [SMILES: Cc1nc2cc(C3CCOC(c4cnn(C5CC5)c4)C3)nc(-c3ccc(F)cc3F)c2nc1CO] | I-1123 |
| 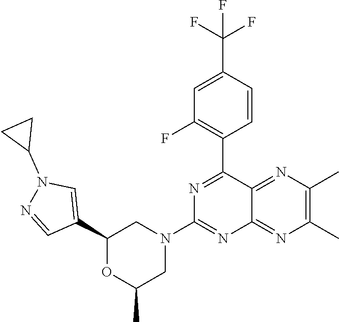 [SMILES: Cc1nc2nc(N3C[C@@H](C)O[C@@H](c4cnn(C5CC5)c4)C3)nc(-c3ccc(C(F)(F)F)cc3F)c2nc1C] | I-1124 |
| 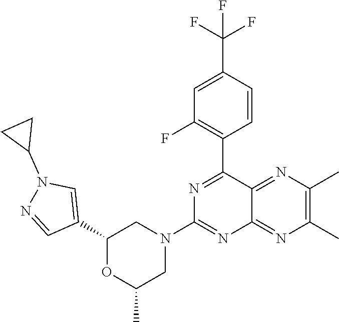 [SMILES: Cc1nc2nc(N3C[C@@H](c4cnn(C5CC5)c4)O[C@@H](C)C3)nc(-c3ccc(C(F)(F)F)cc3F)c2nc1C] | I-1125 |
| 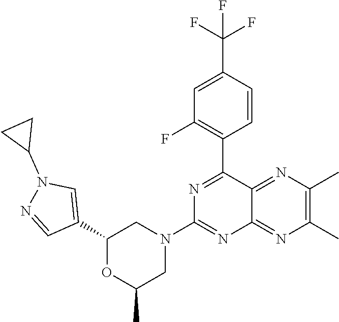 [SMILES: Cc1nc2nc(N3C[C@@H](C)O[C@H](c4cnn(C5CC5)c4)C3)nc(-c3ccc(C(F)(F)F)cc3F)c2nc1C] | I-1126 |
| 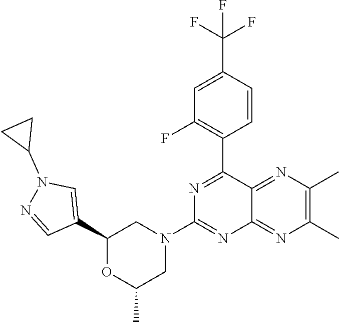 [SMILES: Cc1nc2nc(N3C[C@H](C)O[C@@H](c4cnn(C5CC5)c4)C3)nc(-c3ccc(C(F)(F)F)cc3F)c2nc1C] | I-1127 |
| 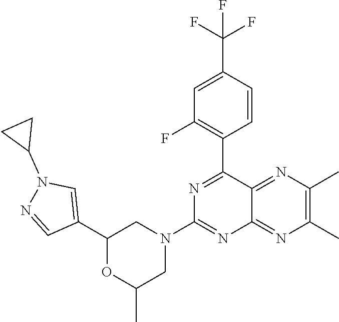 [SMILES: Cc1nc2nc(N3CC(C)OC(c4cnn(C5CC5)c4)C3)nc(-c3ccc(C(F)(F)F)cc3F)c2nc1C] | I-1128 |
| 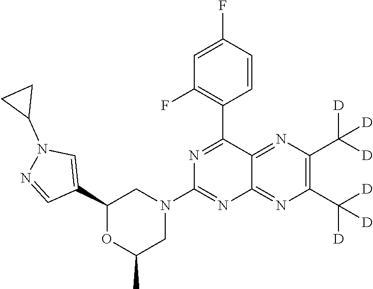 [SMILES: [2H]C([2H])([2H])c1nc2nc(N3C[C@@H](C)O[C@@H](c4cnn(C5CC5)c4)C3)nc(-c3ccc(F)cc3F)c2nc1C([2H])([2H])[2H]] | I-1129 |
| 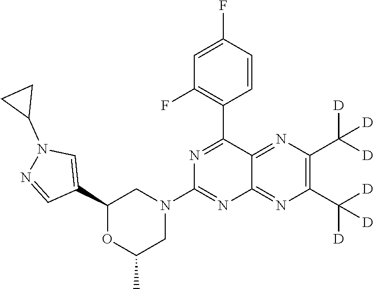 [SMILES: [2H]C([2H])([2H])c1nc2nc(N3C[C@H](C)O[C@@H](c4cnn(C5CC5)c4)C3)nc(-c3ccc(F)cc3F)c2nc1C([2H])([2H])[2H]] | I-1130 |
| 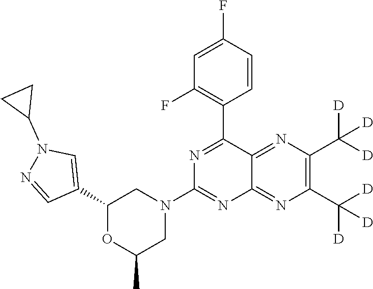 [SMILES: [2H]C([2H])([2H])c1nc2nc(N3C[C@@H](C)O[C@H](c4cnn(C5CC5)c4)C3)nc(-c3ccc(F)cc3F)c2nc1C([2H])([2H])[2H]] | I-1131 |
| 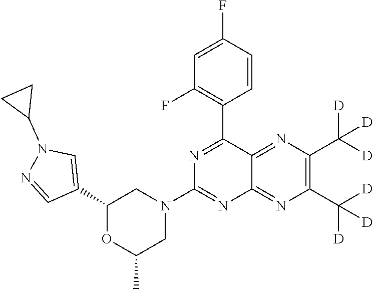 [SMILES: [2H]C([2H])([2H])c1nc2nc(N3C[C@@H](c4cnn(C5CC5)c4)O[C@@H](C)C3)nc(-c3ccc(F)cc3F)c2nc1C([2H])([2H])[2H]] | I-1132 |
| 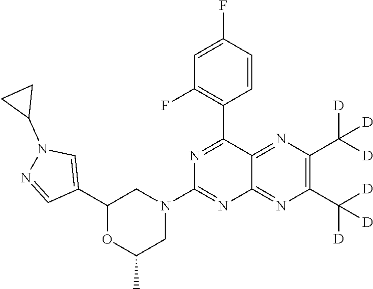 [SMILES: [2H]C([2H])([2H])c1nc2nc(N3CC(c4cnn(C5CC5)c4)O[C@@H](C)C3)nc(-c3ccc(F)cc3F)c2nc1C([2H])([2H])[2H]] | I-1133 |
| 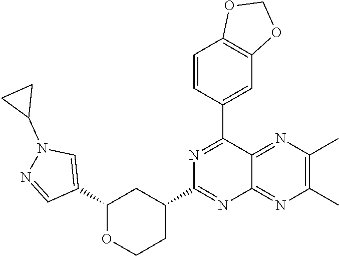 [SMILES: Cc1nc2nc([C@@H]3CCO[C@H](c4cnn(C5CC5)c4)C3)nc(-c3ccc4c(c3)OCO4)c2nc1C] | I-1134 |
| 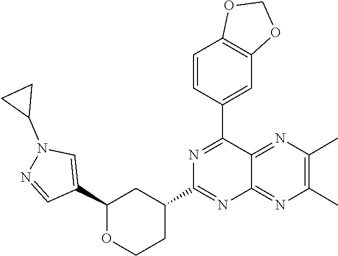 [SMILES: Cc1nc2nc([C@@H]3CCO[C@@H](c4cnn(C5CC5)c4)C3)nc(-c3ccc4c(c3)OCO4)c2nc1C] | I-1135 |
| 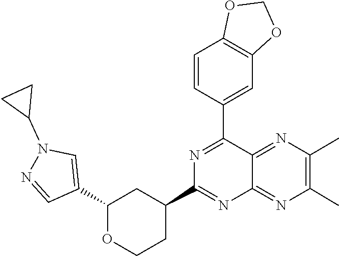 [SMILES: Cc1nc2nc([C@H]3CCO[C@H](c4cnn(C5CC5)c4)C3)nc(-c3ccc4c(c3)OCO4)c2nc1C] | I-1136 |
| 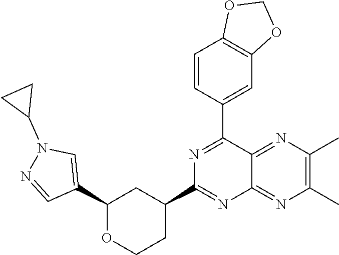 [SMILES: Cc1nc2nc([C@H]3CCO[C@@H](c4cnn(C5CC5)c4)C3)nc(-c3ccc4c(c3)OCO4)c2nc1C] | I-1137 |
| 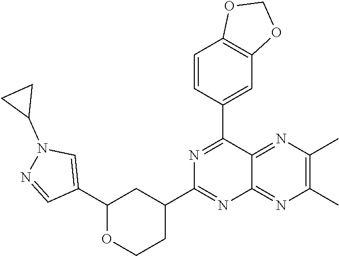 [SMILES: Cc1nc2nc(C3CCOC(c4cnn(C5CC5)c4)C3)nc(-c3ccc4c(c3)OCO4)c2nc1C] | I-1138 |
| 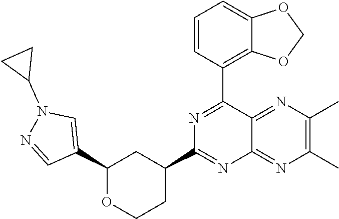 [SMILES: Cc1nc2nc([C@H]3CCO[C@@H](c4cnn(C5CC5)c4)C3)nc(-c3cccc4c3OCO4)c2nc1C] | I-1139 |
| 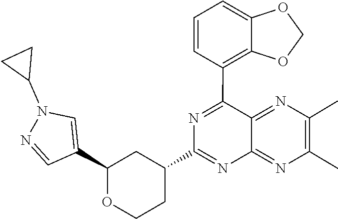 [SMILES: Cc1nc2nc([C@@H]3CCO[C@@H](c4cnn(C5CC5)c4)C3)nc(-c3cccc4c3OCO4)c2nc1C] | I-1140 |
| 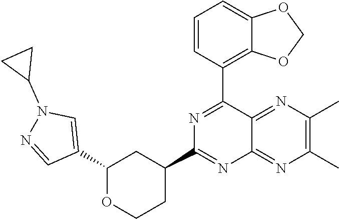 [SMILES: Cc1nc2nc([C@H]3CCO[C@H](c4cnn(C5CC5)c4)C3)nc(-c3cccc4c3OCO4)c2nc1C] | I-1141 |
| 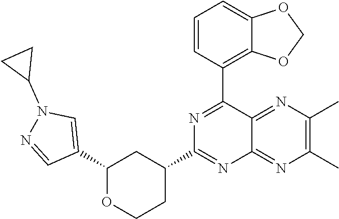 [SMILES: Cc1nc2nc([C@@H]3CCO[C@H](c4cnn(C5CC5)c4)C3)nc(-c3cccc4c3OCO4)c2nc1C] | I-1142 |
| 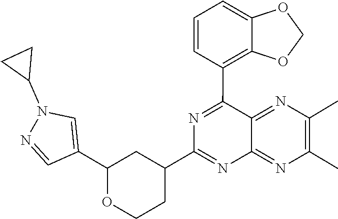 [SMILES: Cc1nc2nc(C3CCOC(c4cnn(C5CC5)c4)C3)nc(-c3cccc4c3OCO4)c2nc1C] | I-1143 |
| 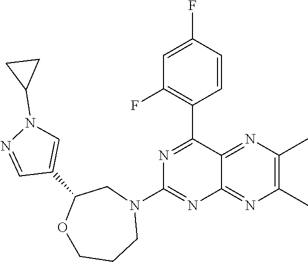 [SMILES: Cc1nc2nc(N3CCCO[C@H](c4cnn(C5CC5)c4)C3)nc(-c3ccc(F)cc3F)c2nc1C] | I-1144 |
| 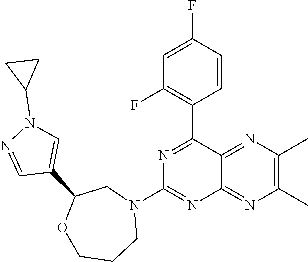 [SMILES: Cc1nc2nc(N3CCCO[C@@H](c4cnn(C5CC5)c4)C3)nc(-c3ccc(F)cc3F)c2nc1C] | I-1145 |
| 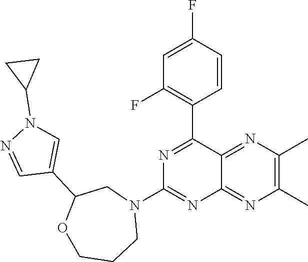 [SMILES: Cc1nc2nc(N3CCCOC(c4cnn(C5CC5)c4)C3)nc(-c3ccc(F)cc3F)c2nc1C] | I-1146 |
| 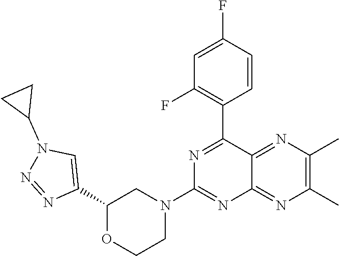 [SMILES: Cc1nc2nc(N3CCO[C@H](c4cn(C5CC5)nn4)C3)nc(-c3ccc(F)cc3F)c2nc1C] | I-1147 |
| 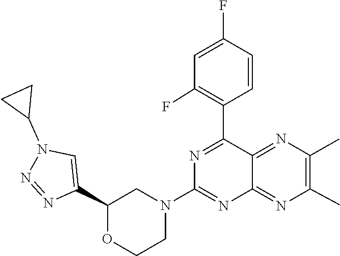 [SMILES: Cc1nc2nc(N3CCO[C@@H](c4cn(C5CC5)nn4)C3)nc(-c3ccc(F)cc3F)c2nc1C] | I-1148 |
| 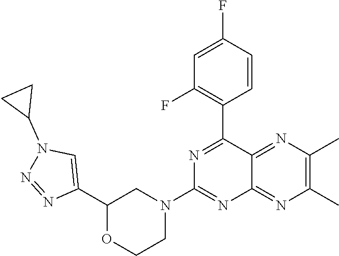 [SMILES: Cc1nc2nc(N3CCOC(c4cn(C5CC5)nn4)C3)nc(-c3ccc(F)cc3F)c2nc1C] | I-1149 |
| 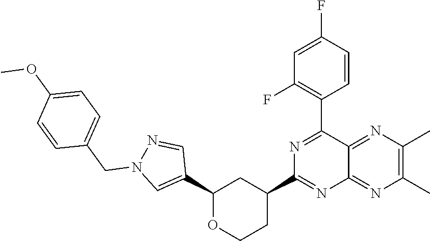 | I-1150 |
| 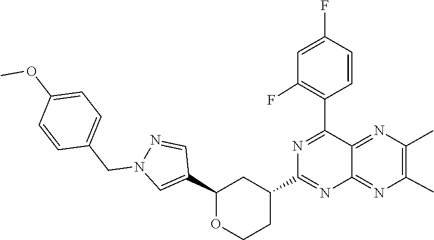 | I-1151 |
| 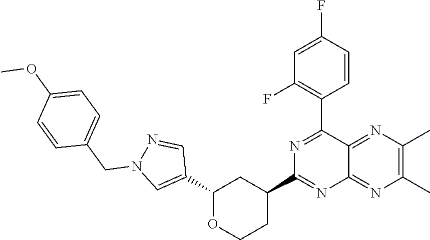 | I-1152 |
| 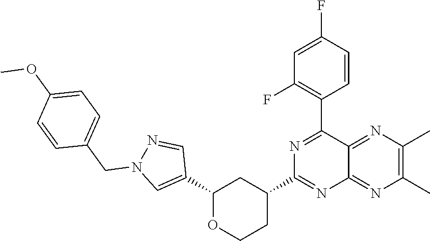 | I-1153 |
| 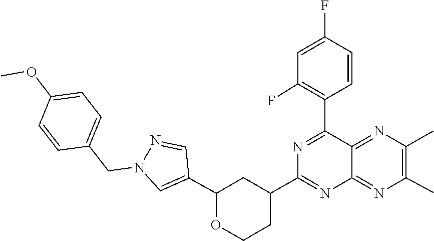 | I-1154 |
| 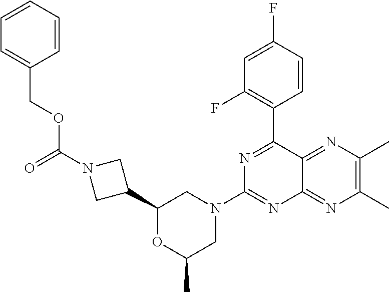 [SMILES: Cc1nc2nc(N3C[C@@H](C)O[C@@H](C4CN(C(=O)OCc5ccccc5)C4)C3)nc(-c3ccc(F)cc3F)c2nc1C] | I-1155 |
| 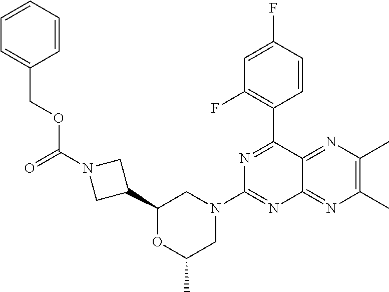 [SMILES: Cc1nc2nc(N3C[C@H](C)O[C@@H](C4CN(C(=O)OCc5ccccc5)C4)C3)nc(-c3ccc(F)cc3F)c2nc1C] | I-1156 |
| 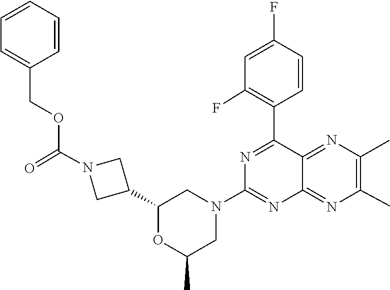 [SMILES: Cc1nc2nc(N3C[C@@H](C)O[C@H](C4CN(C(=O)OCc5ccccc5)C4)C3)nc(-c3ccc(F)cc3F)c2nc1C] | I-1157 |
| 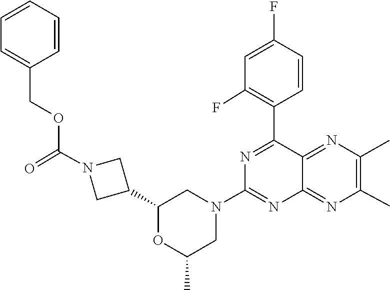 [SMILES: Cc1nc2nc(N3C[C@@H](C4CN(C(=O)OCc5ccccc5)C4)O[C@@H](C)C3)nc(-c3ccc(F)cc3F)c2nc1C] | I-1158 |
| 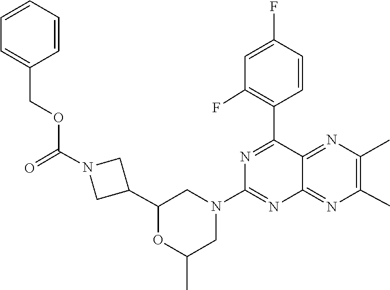 [SMILES: Cc1nc2nc(N3CC(C)OC(C4CN(C(=O)OCc5ccccc5)C4)C3)nc(-c3ccc(F)cc3F)c2nc1C] | I-1159 |
| 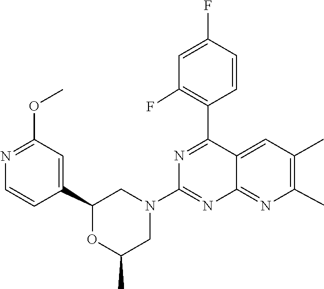 [SMILES: COc1cc([C@H]2CN(c3nc(-c4ccc(F)cc4F)c4cc(C)c(C)nc4n3)C[C@@H](C)O2)ccn1] | I-1160 |
| 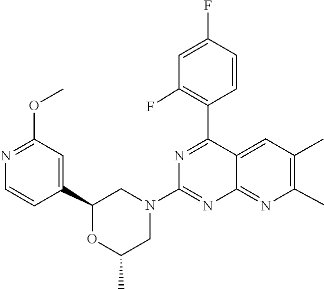 [SMILES: COc1cc([C@H]2CN(c3nc(-c4ccc(F)cc4F)c4cc(C)c(C)nc4n3)C[C@H](C)O2)ccn1] | I-1161 |
| 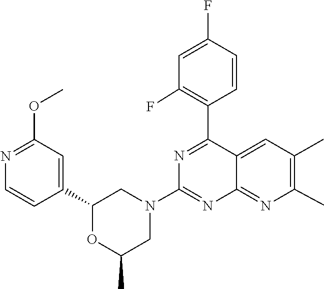 [SMILES: COc1cc([C@@H]2CN(c3nc(-c4ccc(F)cc4F)c4cc(C)c(C)nc4n3)C[C@@H](C)O2)ccn1] | I-1162 |
| 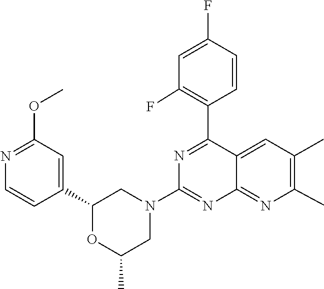 [SMILES: COc1cc([C@@H]2CN(c3nc(-c4ccc(F)cc4F)c4cc(C)c(C)nc4n3)C[C@H](C)O2)ccn1] | I-1163 |
| 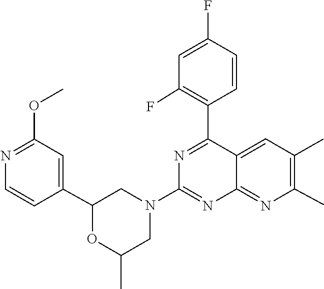 [SMILES: COc1cc(C2CN(c3nc(-c4ccc(F)cc4F)c4cc(C)c(C)nc4n3)CC(C)O2)ccn1] | I-1164 |
| 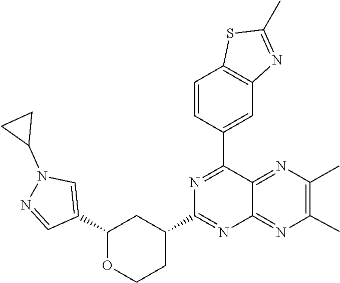 [SMILES: Cc1nc2cc(-c3nc([C@@H]4CCO[C@H](c5cnn(C6CC6)c5)C4)nc4nc(C)c(C)nc34)ccc2s1] | I-1165 |
| 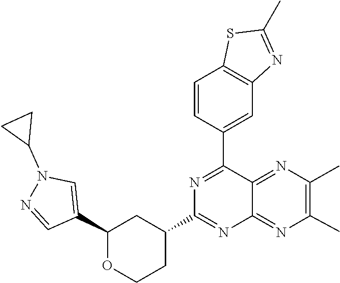 [SMILES: Cc1nc2cc(-c3nc([C@@H]4CCO[C@@H](c5cnn(C6CC6)c5)C4)nc4nc(C)c(C)nc34)ccc2s1] | I-1166 |
| 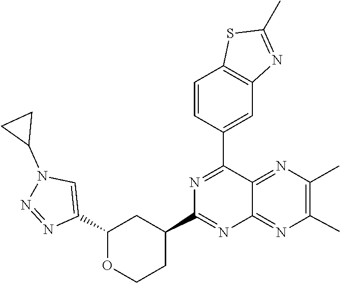 [SMILES: Cc1nc2cc(-c3nc([C@H]4CCO[C@H](c5cn(C6CC6)nn5)C4)nc4nc(C)c(C)nc34)ccc2s1] | I-1167 |
| 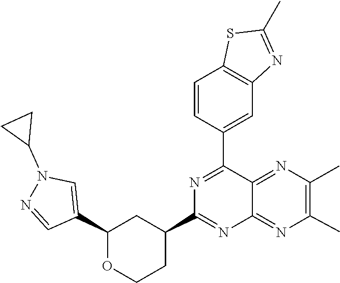 [SMILES: Cc1nc2cc(-c3nc([C@H]4CCO[C@@H](c5cnn(C6CC6)c5)C4)nc4nc(C)c(C)nc34)ccc2s1] | I-1168 |
| 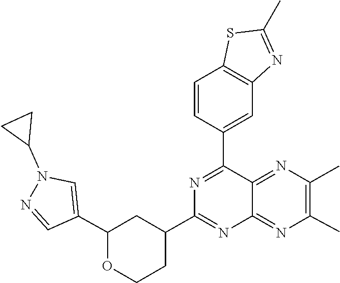 [SMILES: Cc1nc2cc(-c3nc(C4CCOC(c5cnn(C6CC6)c5)C4)nc4nc(C)c(C)nc34)ccc2s1] | I-1169 |
| 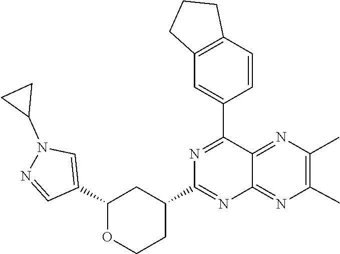 [SMILES: Cc1nc2nc([C@@H]3CCO[C@H](c4cnn(C5CC5)c4)C3)nc(-c3ccc4c(c3)CCC4)c2nc1C] | I-1170 |
| 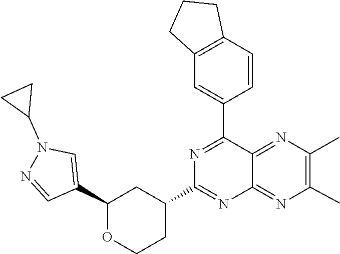 [SMILES: Cc1nc2nc([C@@H]3CCO[C@@H](c4cnn(C5CC5)c4)C3)nc(-c3ccc4c(c3)CCC4)c2nc1C] | I-1171 |
| 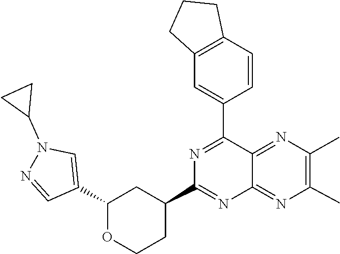 [SMILES: Cc1nc2nc([C@H]3CCO[C@H](c4cnn(C5CC5)c4)C3)nc(-c3ccc4c(c3)CCC4)c2nc1C] | I-1172 |
| 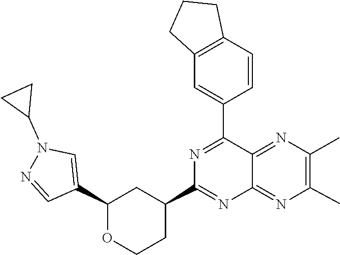 [SMILES: Cc1nc2nc([C@H]3CCO[C@@H](c4cnn(C5CC5)c4)C3)nc(-c3ccc4c(c3)CCC4)c2nc1C] | I-1173 |
| 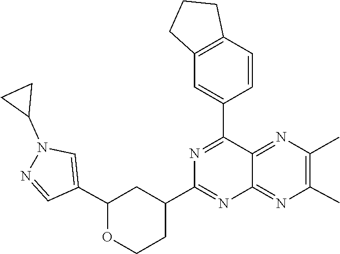 [SMILES: Cc1nc2nc(C3CCOC(c4cnn(C5CC5)c4)C3)nc(-c3ccc4c(c3)CCC4)c2nc1C] | I-1174 |
| 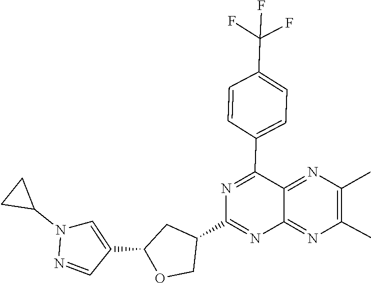 [SMILES: Cc1nc2nc([C@@H]3CO[C@H](c4cnn(C5CC5)c4)C3)nc(-c3ccc(C(F)(F)F)cc3)c2nc1C] | I-1175 |
| 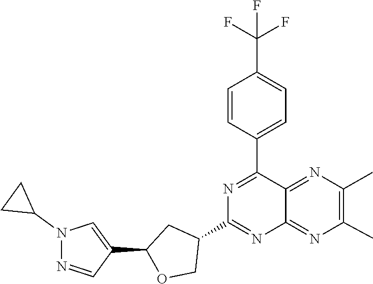 [SMILES: Cc1nc2nc([C@@H]3CO[C@@H](c4cnn(C5CC5)c4)C3)nc(-c3ccc(C(F)(F)F)cc3)c2nc1C] | I-1176 |
| 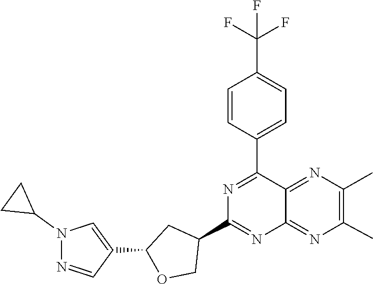 [SMILES: Cc1nc2nc([C@H]3CO[C@H](c4cnn(C5CC5)c4)C3)nc(-c3ccc(C(F)(F)F)cc3)c2nc1C] | I-1177 |
| 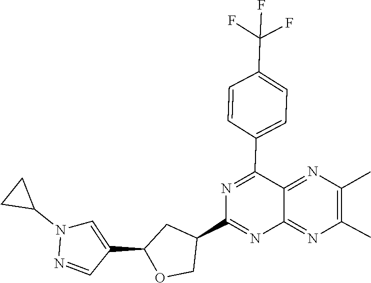 [SMILES: Cc1nc2nc([C@H]3CO[C@@H](c4cnn(C5CC5)c4)C3)nc(-c3ccc(C(F)(F)F)cc3)c2nc1C] | I-1178 |
| 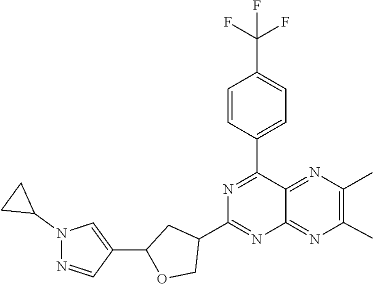 [SMILES: Cc1nc2nc(C3COC(c4cnn(C5CC5)c4)C3)nc(-c3ccc(C(F)(F)F)cc3)c2nc1C] | I-1179 |
| 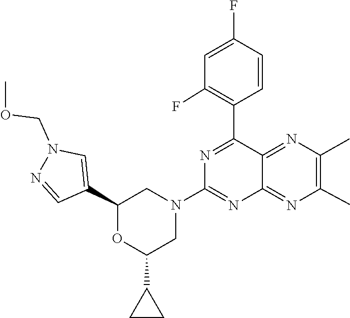 [SMILES: COCn1cc([C@H]2CN(c3nc(-c4ccc(F)cc4F)c4nc(C)c(C)nc4n3)C[C@H](C3CC3)O2)cn1] | I-1180 |
| 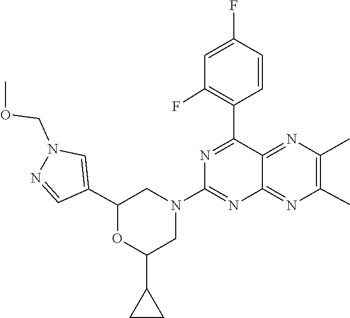 [SMILES: COCn1cc(C2CN(c3nc(-c4ccc(F)cc4F)c4nc(C)c(C)nc4n3)CC(C3CC3)O2)cn1] | I-1181 |
| 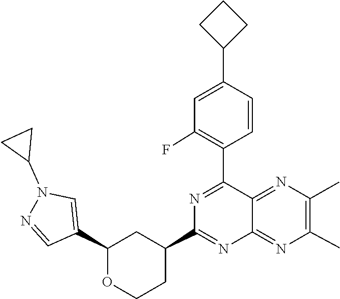 [SMILES: Cc1nc2nc([C@H]3CCO[C@@H](c4cnn(C5CC5)c4)C3)nc(-c3ccc(C4CCC4)cc3F)c2nc1C] | I-1182 |
| 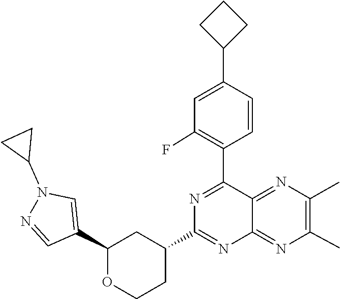 [SMILES: Cc1nc2nc([C@@H]3CCO[C@@H](c4cnn(C5CC5)c4)C3)nc(-c3ccc(C4CCC4)cc3F)c2nc1C] | I-1183 |
| 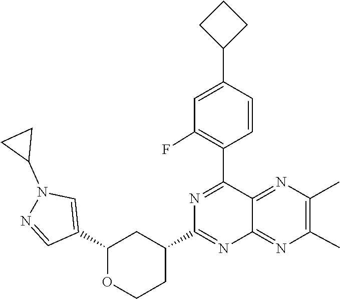 [SMILES: Cc1nc2nc([C@@H]3CCO[C@H](c4cnn(C5CC5)c4)C3)nc(-c3ccc(C4CCC4)cc3F)c2nc1C] | I-1184 |
| 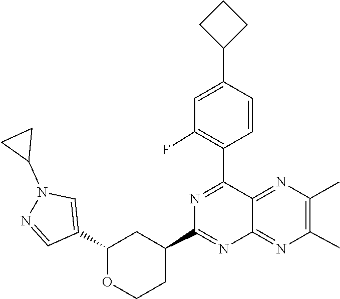 [SMILES: Cc1nc2nc([C@H]3CCO[C@H](c4cnn(C5CC5)c4)C3)nc(-c3ccc(C4CCC4)cc3F)c2nc1C] | I-1185 |
| 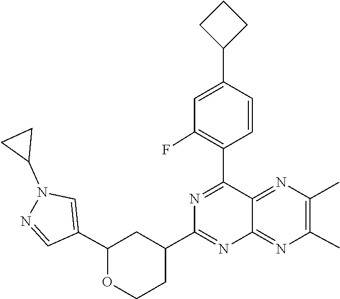 [SMILES: Cc1nc2nc(C3CCOC(c4cnn(C5CC5)c4)C3)nc(-c3ccc(C4CCC4)cc3F)c2nc1C] | I-1186 |
| 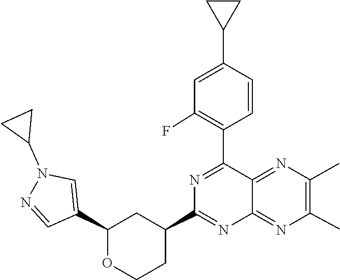 [SMILES: Cc1nc2nc([C@H]3CCO[C@@H](c4cnn(C5CC5)c4)C3)nc(-c3ccc(C4CC4)cc3F)c2nc1C] | I-1187 |
| 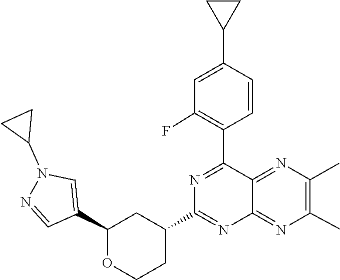 [SMILES: Cc1nc2nc([C@@H]3CCO[C@@H](c4cnn(C5CC5)c4)C3)nc(-c3ccc(C4CC4)cc3F)c2nc1C] | I-1188 |
| 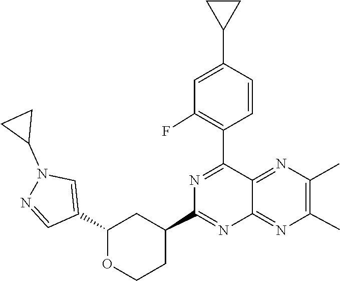 [SMILES: Cc1nc2nc([C@H]3CCO[C@H](c4cnn(C5CC5)c4)C3)nc(-c3ccc(C4CC4)cc3F)c2nc1C] | I-1189 |
| 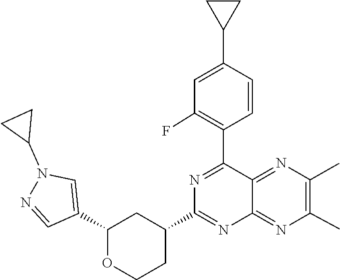 [SMILES: Cc1nc2nc([C@@H]3CCO[C@H](c4cnn(C5CC5)c4)C3)nc(-c3ccc(C4CC4)cc3F)c2nc1C] | I-1190 |
| 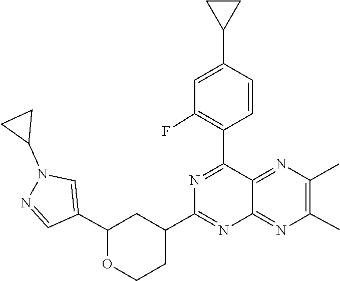 [SMILES: Cc1nc2nc(C3CCOC(c4cnn(C5CC5)c4)C3)nc(-c3ccc(C4CC4)cc3F)c2nc1C] | I-1191 |
| 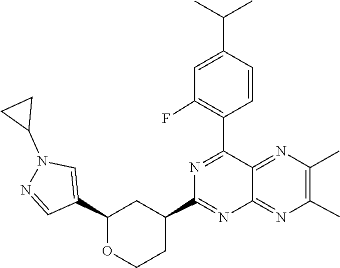 [SMILES: Cc1nc2nc([C@H]3CCO[C@@H](c4cnn(C5CC5)c4)C3)nc(-c3ccc(C(C)C)cc3F)c2nc1C] | I-1192 |
| 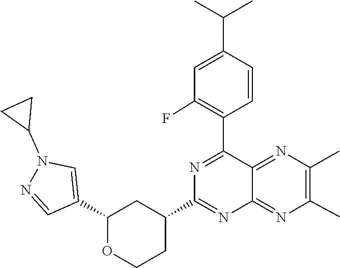 [SMILES: Cc1nc2nc([C@@H]3CCO[C@H](c4cnn(C5CC5)c4)C3)nc(-c3ccc(C(C)C)cc3F)c2nc1C] | I-1193 |
| 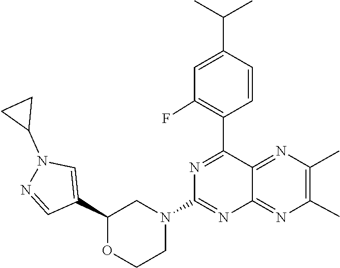 | I-1194 |
| 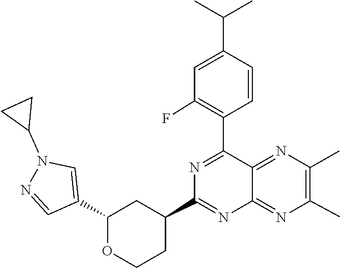 [SMILES: Cc1nc2nc([C@H]3CCO[C@H](c4cnn(C5CC5)c4)C3)nc(-c3ccc(C(C)C)cc3F)c2nc1C] | I-1195 |
| 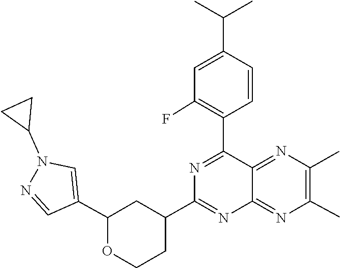 [SMILES: Cc1nc2nc(C3CCOC(c4cnn(C5CC5)c4)C3)nc(-c3ccc(C(C)C)cc3F)c2nc1C] | I-1196 |
| 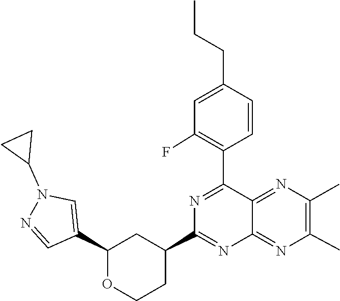 [SMILES: CCCc1ccc(-c2nc([C@H]3CCO[C@@H](c4cnn(C5CC5)c4)C3)nc3nc(C)c(C)nc23)c(F)c1] | I-1197 |
| 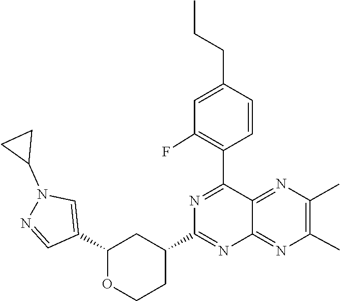 [SMILES: CCCc1ccc(-c2nc([C@@H]3CCO[C@H](c4cnn(C5CC5)c4)C3)nc3nc(C)c(C)nc23)c(F)c1] | I-1198 |
| 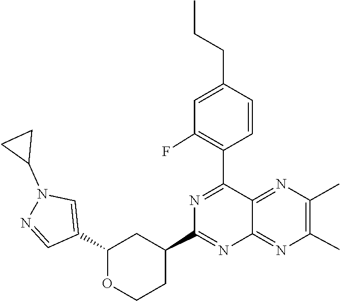 [SMILES: CCCc1ccc(-c2nc([C@H]3CCO[C@H](c4cnn(C5CC5)c4)C3)nc3nc(C)c(C)nc23)c(F)c1] | I-1199 |
| 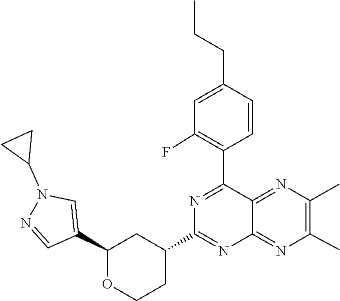 [SMILES: CCCc1ccc(-c2nc([C@@H]3CCO[C@@H](c4cnn(C5CC5)c4)C3)nc3nc(C)c(C)nc23)c(F)c1] | I-1200 |
| 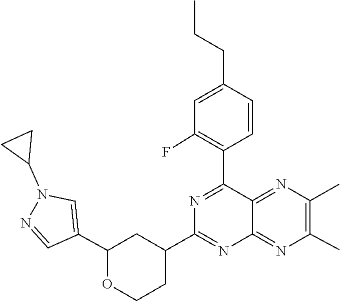 [SMILES: CCCc1ccc(-c2nc(C3CCOC(c4cnn(C5CC5)c4)C3)nc3nc(C)c(C)nc23)c(F)c1] | I-1201 |
| 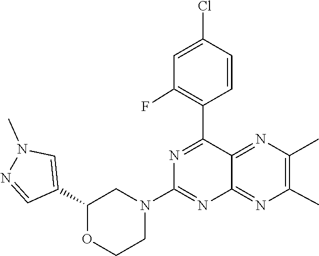 [SMILES: Cc1nc2nc(N3CCO[C@H](c4cnn(C)c4)C3)nc(-c3ccc(Cl)cc3F)c2nc1C] | I-1202 |
| 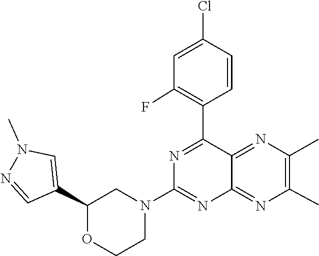 [SMILES: Cc1nc2nc(N3CCO[C@@H](c4cnn(C)c4)C3)nc(-c3ccc(Cl)cc3F)c2nc1C] | I-1203 |
| 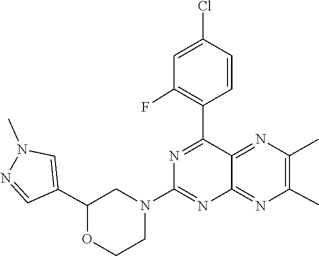 [SMILES: Cc1nc2nc(N3CCOC(c4cnn(C)c4)C3)nc(-c3ccc(Cl)cc3F)c2nc1C] | I-1204 |
| 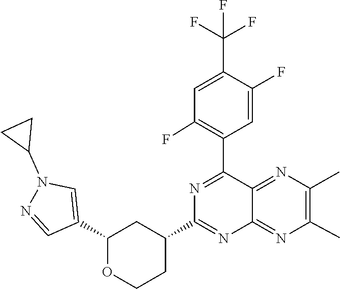 [SMILES: Cc1nc2nc([C@@H]3CCO[C@H](c4cnn(C5CC5)c4)C3)nc(-c3cc(F)c(C(F)(F)F)cc3F)c2nc1C] | I-1205 |
| 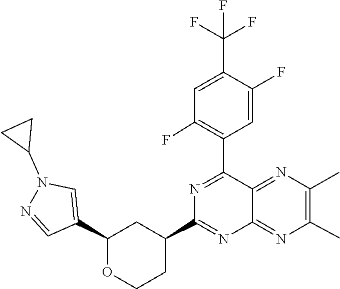 [SMILES: Cc1nc2nc([C@H]3CCO[C@@H](c4cnn(C5CC5)c4)C3)nc(-c3cc(F)c(C(F)(F)F)cc3F)c2nc1C] | I-1206 |
| 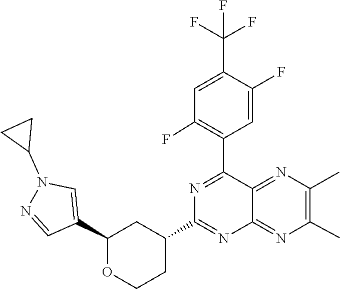 [SMILES: Cc1nc2nc([C@@H]3CCO[C@@H](c4cnn(C5CC5)c4)C3)nc(-c3cc(F)c(C(F)(F)F)cc3F)c2nc1C] | I-1207 |
| 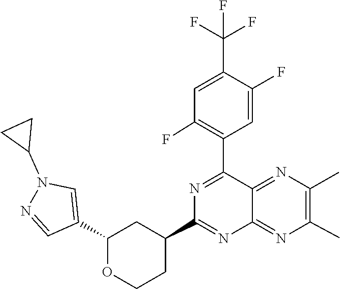 [SMILES: Cc1nc2nc([C@H]3CCO[C@H](c4cnn(C5CC5)c4)C3)nc(-c3cc(F)c(C(F)(F)F)cc3F)c2nc1C] | I-1208 |
| 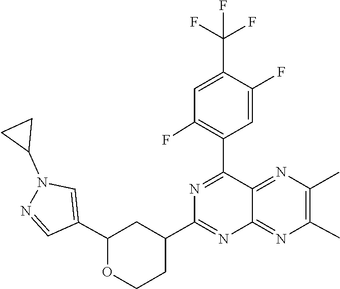 [SMILES: Cc1nc2nc(C3CCOC(c4cnn(C5CC5)c4)C3)nc(-c3cc(F)c(C(F)(F)F)cc3F)c2nc1C] | I-1209 |
| 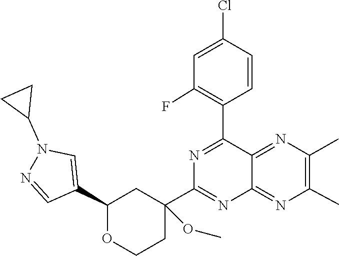 [SMILES: COC1(c2nc(-c3ccc(Cl)cc3F)c3nc(C)c(C)nc3n2)CCO[C@@H](c2cnn(C3CC3)c2)C1] | I-1210 |
| 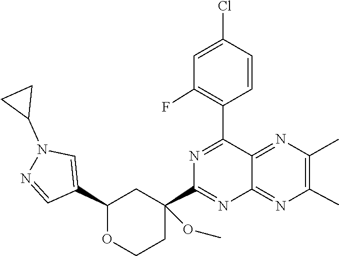 [SMILES: CO[C@]1(c2nc(-c3ccc(Cl)cc3F)c3nc(C)c(C)nc3n2)CCO[C@@H](c2cnn(C3CC3)c2)C1] | I-1211 |
| 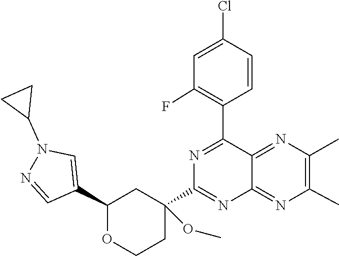 [SMILES: CO[C@@]1(c2nc(-c3ccc(Cl)cc3F)c3nc(C)c(C)nc3n2)CCO[C@@H](c2cnn(C3CC3)c2)C1] | I-1212 |
| 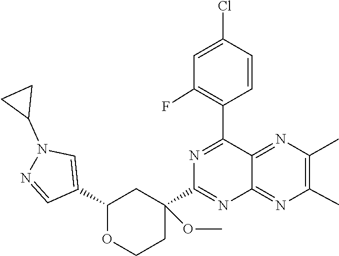 [SMILES: CO[C@@]1(c2nc(-c3ccc(Cl)cc3F)c3nc(C)c(C)nc3n2)CCO[C@H](c2cnn(C3CC3)c2)C1] | I-1213 |
| 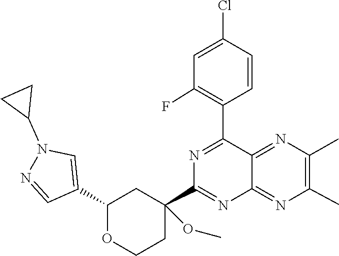 [SMILES: CO[C@]1(c2nc(-c3ccc(Cl)cc3F)c3nc(C)c(C)nc3n2)CCO[C@H](c2cnn(C3CC3)c2)C1] | I-1214 |
| 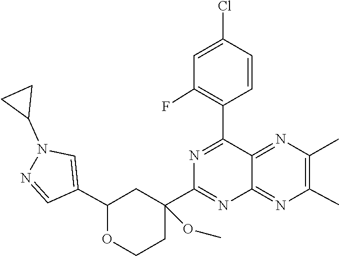 [SMILES: COC1(c2nc(-c3ccc(Cl)cc3F)c3nc(C)c(C)nc3n2)CCOC(c2cnn(C3CC3)c2)C1] | I-1215 |
| 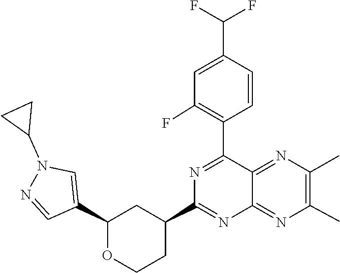 [SMILES: Cc1nc2nc([C@H]3CCO[C@@H](c4cnn(C5CC5)c4)C3)nc(-c3ccc(C(F)F)cc3F)c2nc1C] | I-1216 |
| 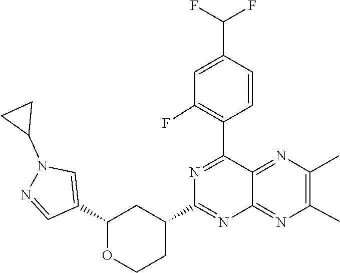 [SMILES: Cc1nc2nc([C@@H]3CCO[C@H](c4cnn(C5CC5)c4)C3)nc(-c3ccc(C(F)F)cc3F)c2nc1C] | I-1217 |
| 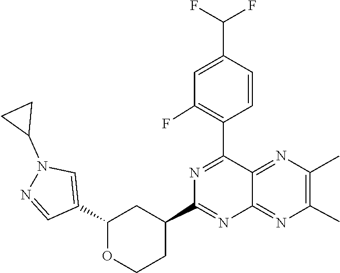 [SMILES: Cc1nc2nc([C@H]3CCO[C@H](c4cnn(C5CC5)c4)C3)nc(-c3ccc(C(F)F)cc3F)c2nc1C] | I-1218 |
| 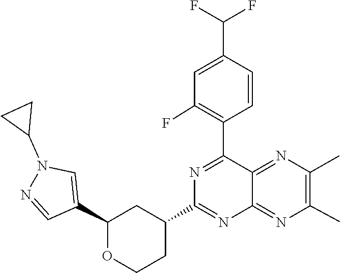 [SMILES: Cc1nc2nc([C@@H]3CCO[C@@H](c4cnn(C5CC5)c4)C3)nc(-c3ccc(C(F)F)cc3F)c2nc1C] | I-1219 |
| 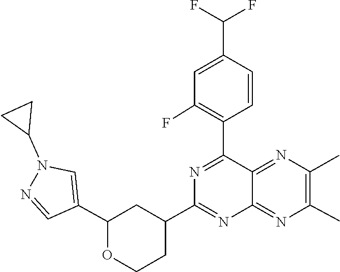 [SMILES: Cc1nc2nc(C3CCOC(c4cnn(C5CC5)c4)C3)nc(-c3ccc(C(F)F)cc3F)c2nc1C] | I-1220 |
| 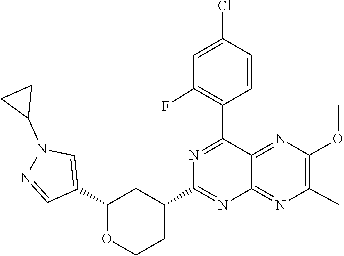 [SMILES: COc1nc2c(-c3ccc(Cl)cc3F)nc([C@@H]3CCO[C@H](c4cnn(C5CC5)c4)C3)nc2nc1C] | I-1221 |
| 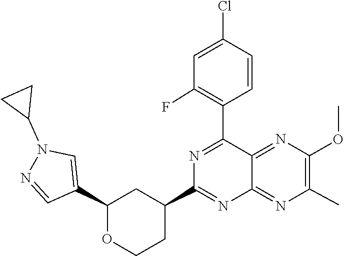 [SMILES: COc1nc2c(-c3ccc(Cl)cc3F)nc([C@H]3CCO[C@@H](c4cnn(C5CC5)c4)C3)nc2nc1C] | I-1222 |
| 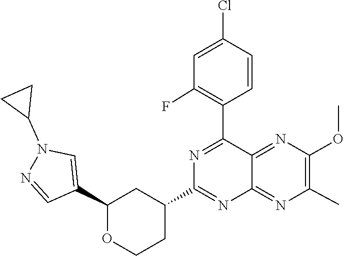 [SMILES: COc1nc2c(-c3ccc(Cl)cc3F)nc([C@@H]3CCO[C@@H](c4cnn(C5CC5)c4)C3)nc2nc1C] | I-1223 |
| 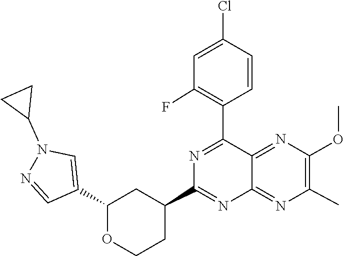 [SMILES: COc1nc2c(-c3ccc(Cl)cc3F)nc([C@H]3CCO[C@H](c4cnn(C5CC5)c4)C3)nc2nc1C] | I-1224 |
| 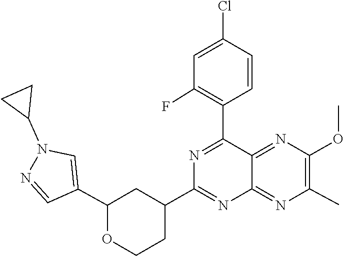 [SMILES: COc1nc2c(-c3ccc(Cl)cc3F)nc(C3CCOC(c4cnn(C5CC5)c4)C3)nc2nc1C] | I-1225 |
| 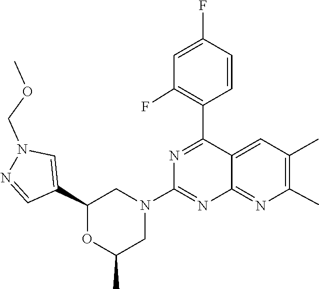 [SMILES: COCn1cc([C@H]2CN(c3nc(-c4ccc(F)cc4F)c4cc(C)c(C)nc4n3)C[C@@H](C)O2)cn1] | I-1226 |
| 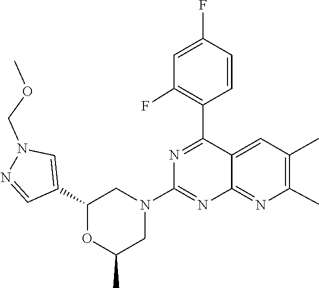 [SMILES: COCn1cc([C@@H]2CN(c3nc(-c4ccc(F)cc4F)c4cc(C)c(C)nc4n3)C[C@@H](C)O2)cn1] | I-1227 |
| 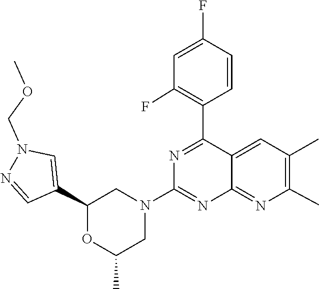 [SMILES: COCn1cc([C@H]2CN(c3nc(-c4ccc(F)cc4F)c4cc(C)c(C)nc4n3)C[C@H](C)O2)cn1] | I-1228 |
| 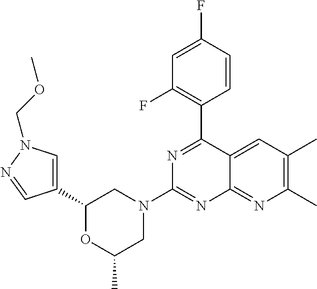 [SMILES: COCn1cc([C@@H]2CN(c3nc(-c4ccc(F)cc4F)c4cc(C)c(C)nc4n3)C[C@H](C)O2)cn1] | I-1229 |
| 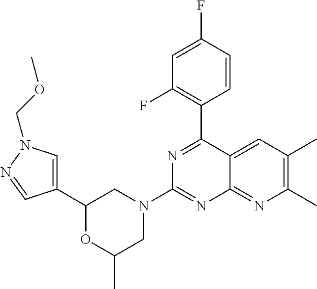 [SMILES: COCn1cc(C2CN(c3nc(-c4ccc(F)cc4F)c4cc(C)c(C)nc4n3)CC(C)O2)cn1] | I-1230 |
| 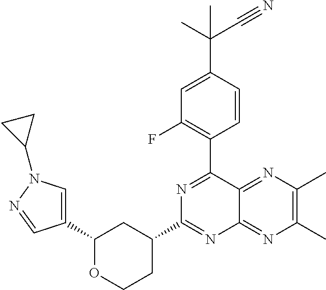 [SMILES: Cc1nc2nc([C@@H]3CCO[C@H](c4cnn(C5CC5)c4)C3)nc(-c3ccc(C(C)(C)C#N)cc3F)c2nc1C] | I-1231 |
| 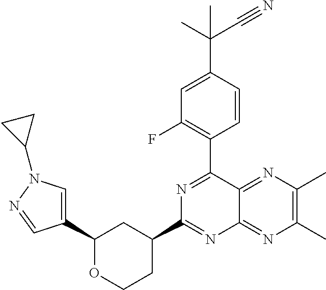 [SMILES: Cc1nc2nc([C@H]3CCO[C@@H](c4cnn(C5CC5)c4)C3)nc(-c3ccc(C(C)(C)C#N)cc3F)c2nc1C] | I-1232 |
| 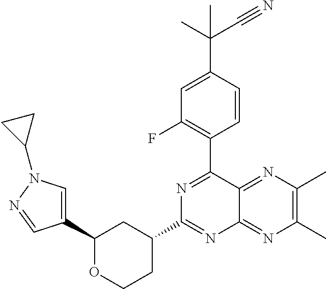 [SMILES: Cc1nc2nc([C@@H]3CCO[C@@H](c4cnn(C5CC5)c4)C3)nc(-c3ccc(C(C)(C)C#N)cc3F)c2nc1C] | I-1233 |
| 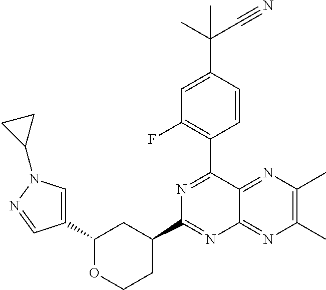 [SMILES: Cc1nc2nc([C@H]3CCO[C@H](c4cnn(C5CC5)c4)C3)nc(-c3ccc(C(C)(C)C#N)cc3F)c2nc1C] | I-1234 |
| 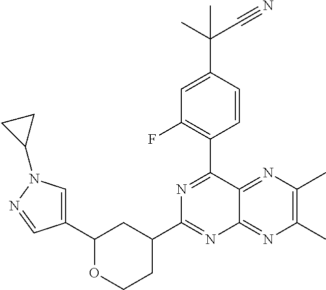 [SMILES: Cc1nc2nc(C3CCOC(c4cnn(C5CC5)c4)C3)nc(-c3ccc(C(C)(C)C#N)cc3F)c2nc1C] | I-1235 |
| 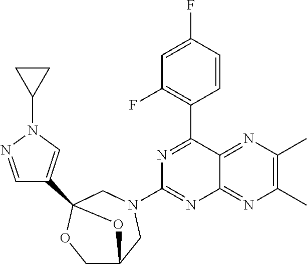 [SMILES: Cc1nc2nc(N3C[C@@H]4CO[C@](c5cnn(C6CC6)c5)(C3)O4)nc(-c3ccc(F)cc3F)c2nc1C] | I-1236 |
| 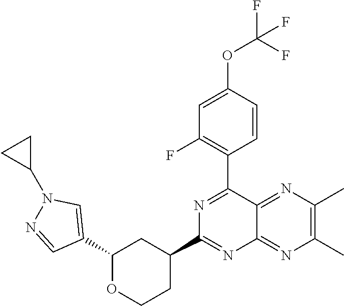 [SMILES: Cc1nc2nc([C@H]3CCO[C@H](c4cnn(C5CC5)c4)C3)nc(-c3ccc(OC(F)(F)F)cc3F)c2nc1C] | I-1237 |
| 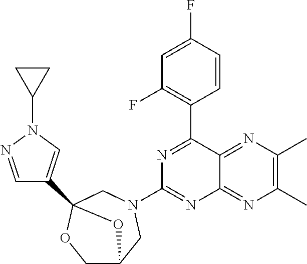 [SMILES: Cc1nc2nc(N3C[C@H]4CO[C@](c5cnn(C6CC6)c5)(C3)O4)nc(-c3ccc(F)cc3F)c2nc1C] | I-1238 |
| 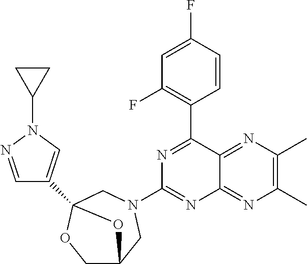 [SMILES: Cc1nc2nc(N3C[C@@H]4CO[C@@](c5cnn(C6CC6)c5)(C3)O4)nc(-c3ccc(F)cc3F)c2nc1C] | I-1239 |
| 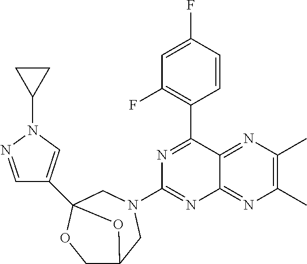 [SMILES: Cc1nc2nc(N3CC4COC(c5cnn(C6CC6)c5)(C3)O4)nc(-c3ccc(F)cc3F)c2nc1C] | I-1240 |
| 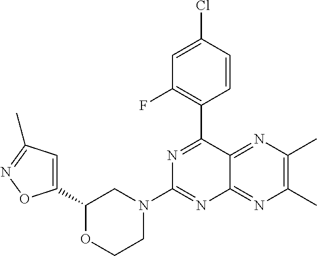 [SMILES: Cc1cc([C@@H]2CN(c3nc(-c4ccc(Cl)cc4F)c4nc(C)c(C)nc4n3)CCO2)on1] | I-1241 |
| 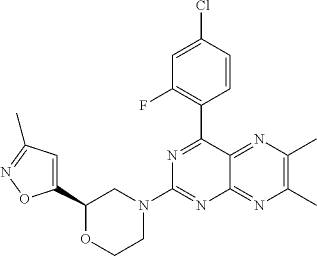 [SMILES: Cc1cc([C@H]2CN(c3nc(-c4ccc(Cl)cc4F)c4nc(C)c(C)nc4n3)CCO2)on1] | I-1242 |
| 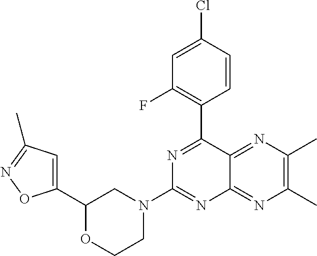 [SMILES: Cc1cc(C2CN(c3nc(-c4ccc(Cl)cc4F)c4nc(C)c(C)nc4n3)CCO2)on1] | I-1243 |
| 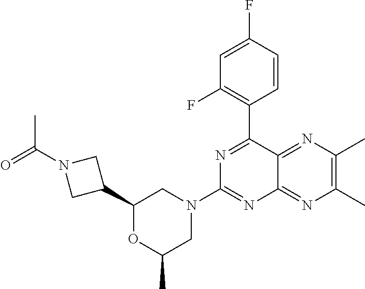 [SMILES: CC(=O)N1CC([C@H]2CN(c3nc(-c4ccc(F)cc4F)c4nc(C)c(C)nc4n3)C[C@@H](C)O2)C1] | I-1244 |
| 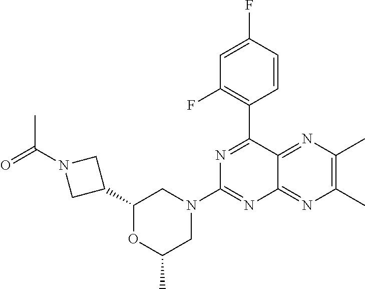 [SMILES: CC(=O)N1CC([C@@H]2CN(c3nc(-c4ccc(F)cc4F)c4nc(C)c(C)nc4n3)C[C@H](C)O2)C1] | I-1245 |
| 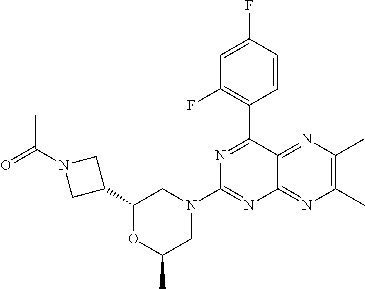 [SMILES: CC(=O)N1CC([C@@H]2CN(c3nc(-c4ccc(F)cc4F)c4nc(C)c(C)nc4n3)C[C@@H](C)O2)C1] | I-1246 |
| 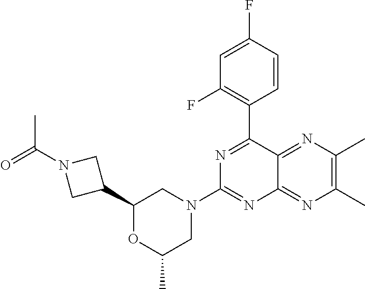 [SMILES: CC(=O)N1CC([C@H]2CN(c3nc(-c4ccc(F)cc4F)c4nc(C)c(C)nc4n3)C[C@H](C)O2)C1] | I-1247 |
| 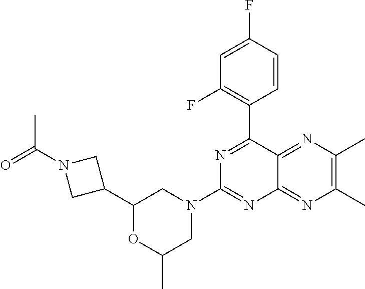 [SMILES: CC(=O)N1CC(C2CN(c3nc(-c4ccc(F)cc4F)c4nc(C)c(C)nc4n3)CC(C)O2)C1] | I-1248 |
| 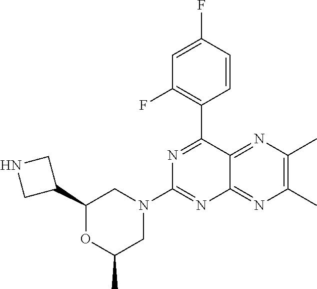 [SMILES: Cc1nc2nc(N3C[C@@H](C)O[C@@H](C4CNC4)C3)nc(-c3ccc(F)cc3F)c2nc1C] | I-1249 |
| 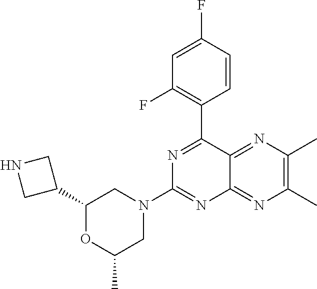 [SMILES: Cc1nc2nc(N3C[C@@H](C4CNC4)O[C@@H](C)C3)nc(-c3ccc(F)cc3F)c2nc1C] | I-1250 |
| 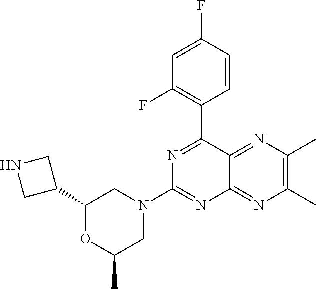 [SMILES: Cc1nc2nc(N3C[C@@H](C)O[C@H](C4CNC4)C3)nc(-c3ccc(F)cc3F)c2nc1C] | I-1251 |
| 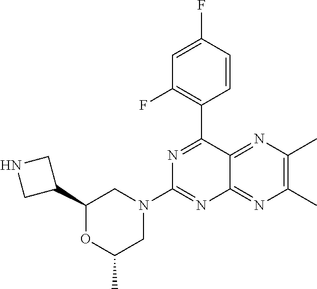 [SMILES: Cc1nc2nc(N3C[C@H](C)O[C@@H](C4CNC4)C3)nc(-c3ccc(F)cc3F)c2nc1C] | I-1252 |
| 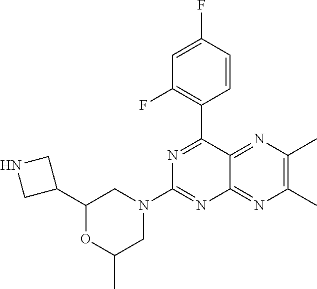 [SMILES: Cc1nc2nc(N3CC(C)OC(C4CNC4)C3)nc(-c3ccc(F)cc3F)c2nc1C] | I-1253 |
| 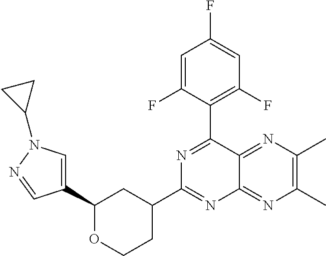 [SMILES: Cc1nc2nc(C3CCO[C@@H](c4cnn(C5CC5)c4)C3)nc(-c3c(F)cc(F)cc3F)c2nc1C] | I-1254 |
| 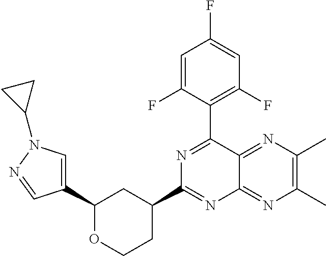 [SMILES: Cc1nc2nc([C@H]3CCO[C@@H](c4cnn(C5CC5)c4)C3)nc(-c3c(F)cc(F)cc3F)c2nc1C] | I-1255 |
| 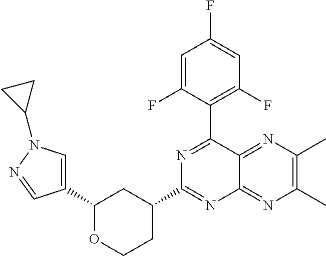 [SMILES: Cc1nc2nc([C@@H]3CCO[C@H](c4cnn(C5CC5)c4)C3)nc(-c3c(F)cc(F)cc3F)c2nc1C] | I-1256 |
| 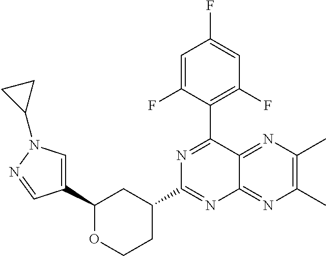 [SMILES: Cc1nc2nc([C@@H]3CCO[C@@H](c4cnn(C5CC5)c4)C3)nc(-c3c(F)cc(F)cc3F)c2nc1C] | I-1257 |
| 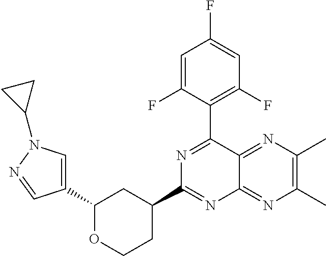 [SMILES: Cc1nc2nc([C@H]3CCO[C@H](c4cnn(C5CC5)c4)C3)nc(-c3c(F)cc(F)cc3F)c2nc1C] | I-1258 |
| 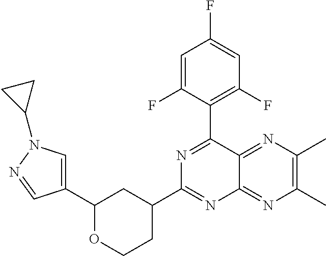 [SMILES: Cc1nc2nc(C3CCOC(c4cnn(C5CC5)c4)C3)nc(-c3c(F)cc(F)cc3F)c2nc1C] | I-1259 |
| 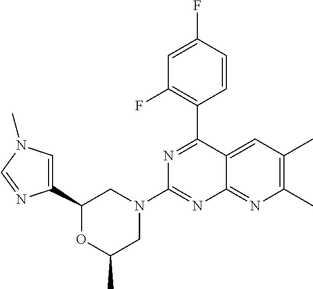 [SMILES: Cc1cc2c(-c3ccc(F)cc3F)nc(N3C[C@@H](C)O[C@@H](c4cn(C)cn4)C3)nc2nc1C] | I-1260 |
| 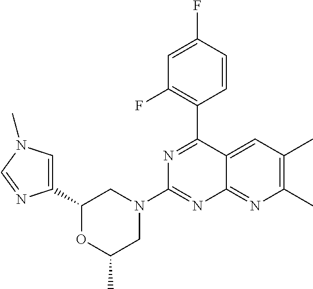 [SMILES: Cc1cc2c(-c3ccc(F)cc3F)nc(N3C[C@@H](c4cn(C)cn4)O[C@@H](C)C3)nc2nc1C] | I-1261 |
| 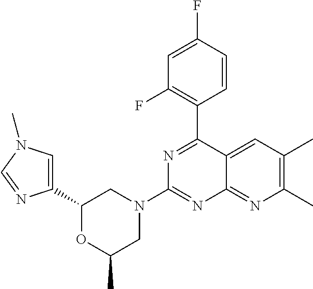 [SMILES: Cc1cc2c(-c3ccc(F)cc3F)nc(N3C[C@@H](C)O[C@H](c4cn(C)cn4)C3)nc2nc1C] | I-1262 |
| 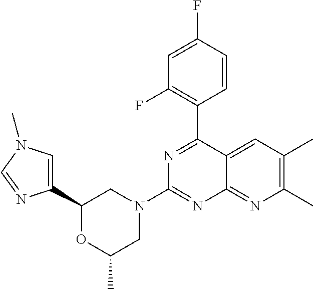 [SMILES: Cc1cc2c(-c3ccc(F)cc3F)nc(N3C[C@H](C)O[C@@H](c4cn(C)cn4)C3)nc2nc1C] | I-1263 |
| 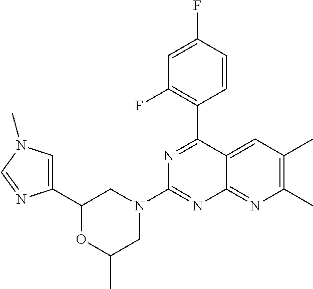 [SMILES: Cc1cc2c(-c3ccc(F)cc3F)nc(N3CC(C)OC(c4cn(C)cn4)C3)nc2nc1C] | I-1264 |
| 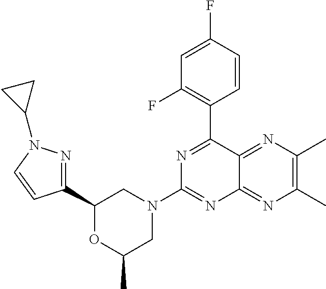 [SMILES: Cc1nc2nc(N3C[C@@H](C)O[C@@H](c4ccn(C5CC5)n4)C3)nc(-c3ccc(F)cc3F)c2nc1C] | I-1265 |
| 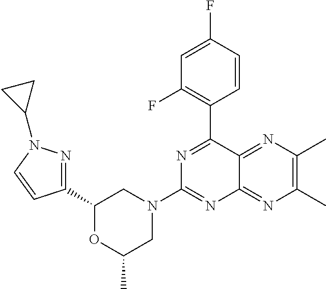 [SMILES: Cc1nc2nc(N3C[C@@H](c4ccn(C5CC5)n4)O[C@@H](C)C3)nc(-c3ccc(F)cc3F)c2nc1C] | I-1266 |
| 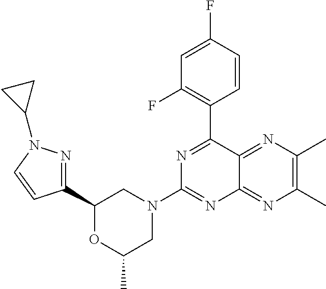 [SMILES: Cc1nc2nc(N3C[C@H](C)O[C@@H](c4ccn(C5CC5)n4)C3)nc(-c3ccc(F)cc3F)c2nc1C] | I-1267 |
| 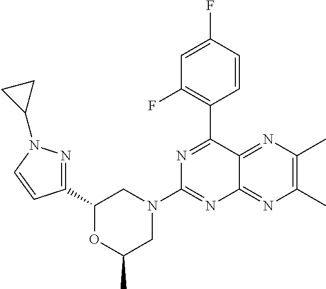 [SMILES: Cc1nc2nc(N3C[C@@H](C)O[C@H](c4ccn(C5CC5)n4)C3)nc(-c3ccc(F)cc3F)c2nc1C] | I-1268 |
| 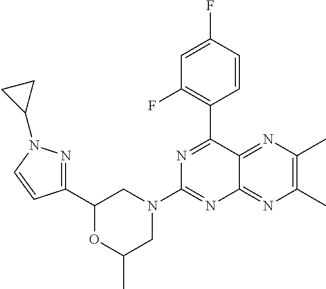 [SMILES: Cc1nc2nc(N3CC(C)OC(c4ccn(C5CC5)n4)C3)nc(-c3ccc(F)cc3F)c2nc1C] | I-1269 |
| 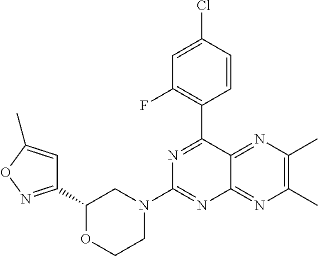 [SMILES: Cc1cc([C@@H]2CN(c3nc(-c4ccc(Cl)cc4F)c4nc(C)c(C)nc4n3)CCO2)no1] | I-1270 |
| 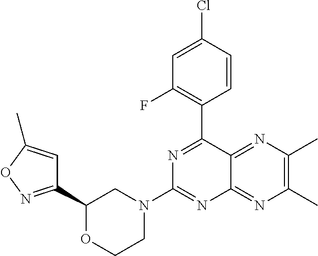 [SMILES: Cc1cc([C@H]2CN(c3nc(-c4ccc(Cl)cc4F)c4nc(C)c(C)nc4n3)CCO2)no1] | I-1271 |
| 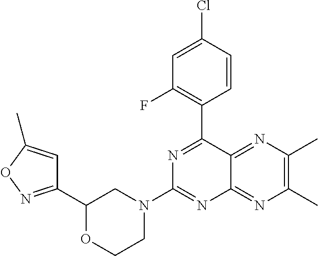 [SMILES: Cc1cc(C2CN(c3nc(-c4ccc(Cl)cc4F)c4nc(C)c(C)nc4n3)CCO2)no1] | I-1272 |
| 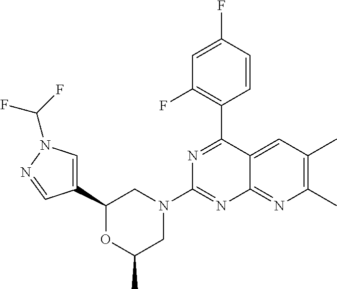 [SMILES: Cc1cc2c(-c3ccc(F)cc3F)nc(N3C[C@@H](C)O[C@@H](c4cnn(C(F)F)c4)C3)nc2nc1C] | I-1273 |
| 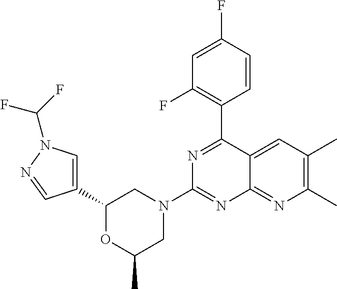 [SMILES: Cc1cc2c(-c3ccc(F)cc3F)nc(N3C[C@@H](C)O[C@H](c4cnn(C(F)F)c4)C3)nc2nc1C] | I-1274 |
| 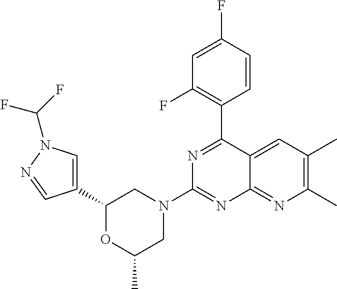 [SMILES: Cc1cc2c(-c3ccc(F)cc3F)nc(N3C[C@@H](c4cnn(C(F)F)c4)O[C@@H](C)C3)nc2nc1C] | I-1275 |
| 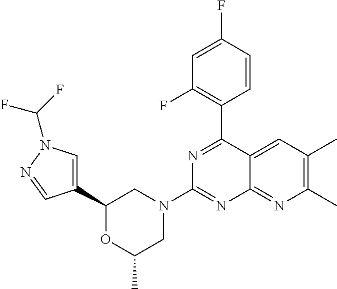 [SMILES: Cc1cc2c(-c3ccc(F)cc3F)nc(N3C[C@H](C)O[C@@H](c4cnn(C(F)F)c4)C3)nc2nc1C] | I-1276 |
| 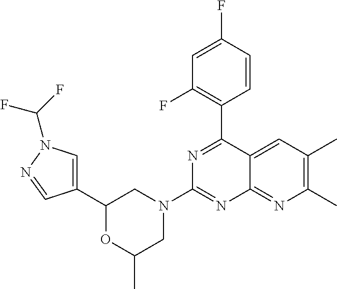 [SMILES: Cc1cc2c(-c3ccc(F)cc3F)nc(N3CC(C)OC(c4cnn(C(F)F)c4)C3)nc2nc1C] | I-1277 |
| 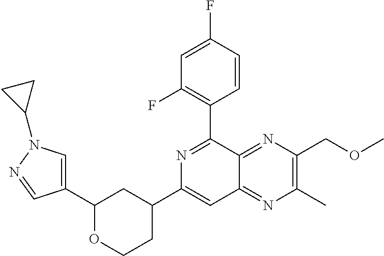 [SMILES: COCc1nc2c(-c3ccc(F)cc3F)nc(C3CCOC(c4cnn(C5CC5)c4)C3)cc2nc1C] | I-1278 |
| 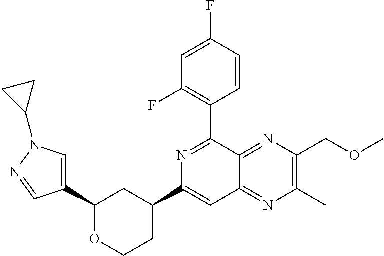 [SMILES: COCc1nc2c(-c3ccc(F)cc3F)nc([C@H]3CCO[C@@H](c4cnn(C5CC5)c4)C3)cc2nc1C] | I-1279 |
| 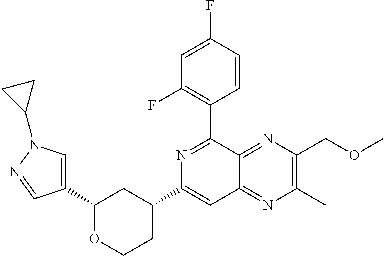 [SMILES: COCc1nc2c(-c3ccc(F)cc3F)nc([C@@H]3CCO[C@H](c4cnn(C5CC5)c4)C3)cc2nc1C] | I-1280 |
| 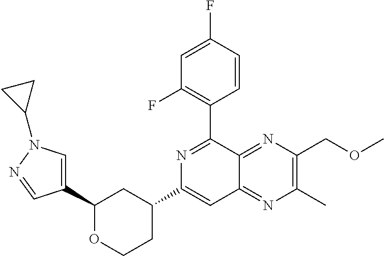 [SMILES: COCc1nc2c(-c3ccc(F)cc3F)nc([C@@H]3CCO[C@@H](c4cnn(C5CC5)c4)C3)cc2nc1C] | I-1281 |
| 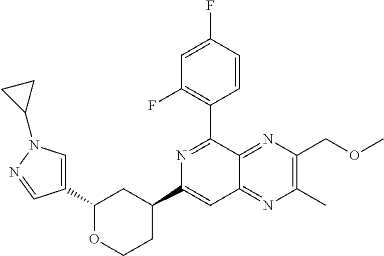 [SMILES: COCc1nc2c(-c3ccc(F)cc3F)nc([C@H]3CCO[C@H](c4cnn(C5CC5)c4)C3)cc2nc1C] | I-1282 |
| 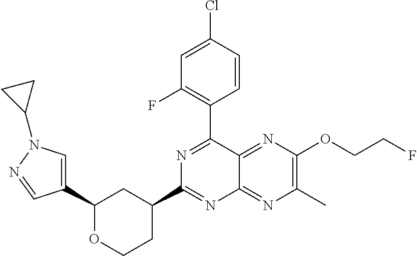 [SMILES: Cc1nc2nc([C@H]3CCO[C@@H](c4cnn(C5CC5)c4)C3)nc(-c3ccc(Cl)cc3F)c2nc1OCCF] | I-1283 |
| 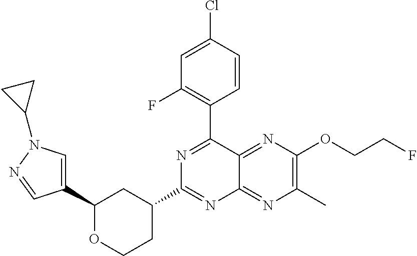 [SMILES: Cc1nc2nc([C@@H]3CCO[C@@H](c4cnn(C5CC5)c4)C3)nc(-c3ccc(Cl)cc3F)c2nc1OCCF] | I-1284 |
| 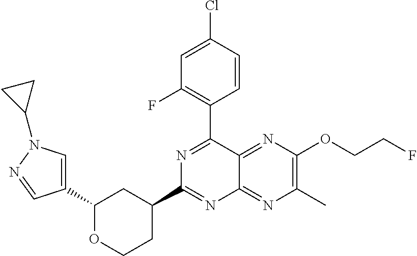 [SMILES: Cc1nc2nc([C@H]3CCO[C@H](c4cnn(C5CC5)c4)C3)nc(-c3ccc(Cl)cc3F)c2nc1OCCF] | I-1285 |
| 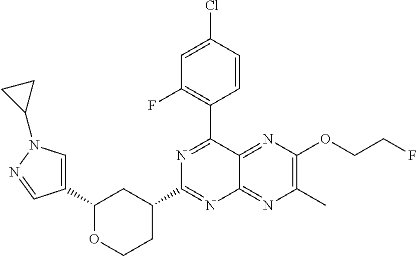 [SMILES: Cc1nc2nc([C@@H]3CCO[C@H](c4cnn(C5CC5)c4)C3)nc(-c3ccc(Cl)cc3F)c2nc1OCCF] | I-1286 |
| 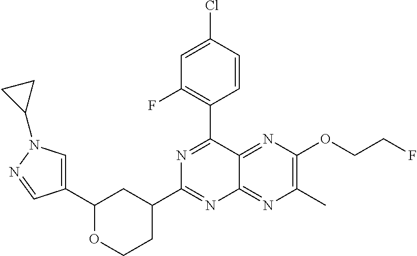 [SMILES: Cc1nc2nc(C3CCOC(c4cnn(C5CC5)c4)C3)nc(-c3ccc(Cl)cc3F)c2nc1OCCF] | I-1287 |
| 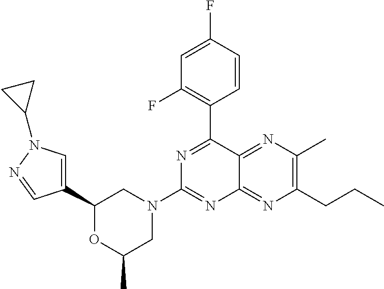 [SMILES: CCCc1nc2nc(N3C[C@@H](C)O[C@@H](c4cnn(C5CC5)c4)C3)nc(-c3ccc(F)cc3F)c2nc1C] | I-1288 |
| 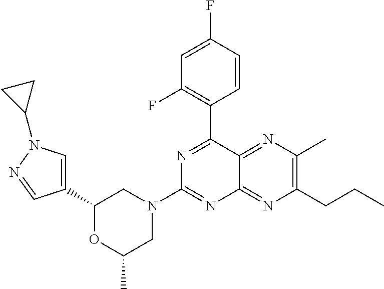 [SMILES: CCCc1nc2nc(N3C[C@@H](c4cnn(C5CC5)c4)O[C@@H](C)C3)nc(-c3ccc(F)cc3F)c2nc1C] | I-1289 |
| 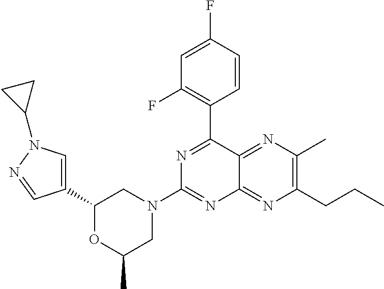 [SMILES: CCCc1nc2nc(N3C[C@@H](C)O[C@H](c4cnn(C5CC5)c4)C3)nc(-c3ccc(F)cc3F)c2nc1C] | I-1290 |
| 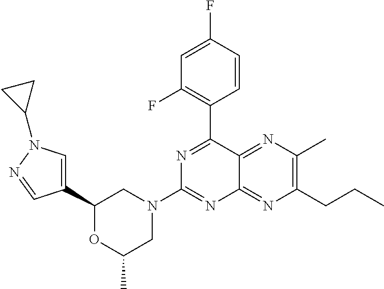 [SMILES: CCCc1nc2nc(N3C[C@H](C)O[C@@H](c4cnn(C5CC5)c4)C3)nc(-c3ccc(F)cc3F)c2nc1C] | I-1291 |
| 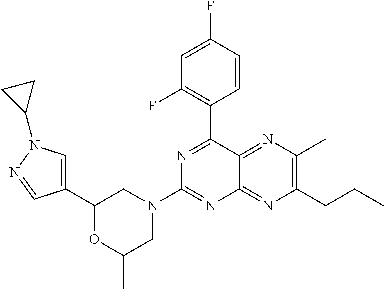 [SMILES: CCCc1nc2nc(N3CC(C)OC(c4cnn(C5CC5)c4)C3)nc(-c3ccc(F)cc3F)c2nc1C] | I-1292 |
| 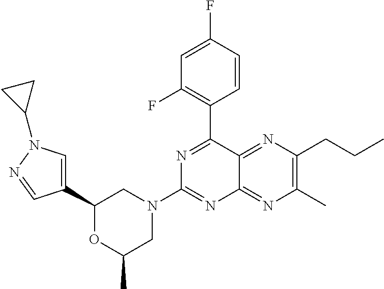 [SMILES: CCCc1nc2c(-c3ccc(F)cc3F)nc(N3C[C@@H](C)O[C@@H](c4cnn(C5CC5)c4)C3)nc2nc1C] | I-1293 |
| 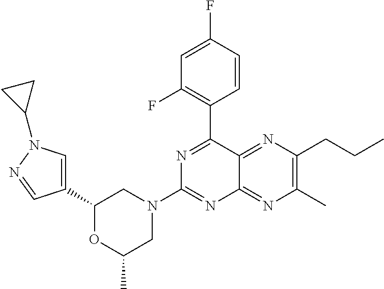 [SMILES: CCCc1nc2c(-c3ccc(F)cc3F)nc(N3C[C@@H](c4cnn(C5CC5)c4)O[C@@H](C)C3)nc2nc1C] | I-1294 |
| 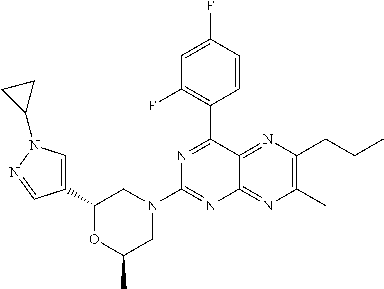 [SMILES: CCCc1nc2c(-c3ccc(F)cc3F)nc(N3C[C@@H](C)O[C@H](c4cnn(C5CC5)c4)C3)nc2nc1C] | I-1295 |
| 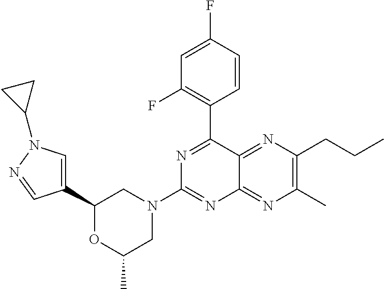 [SMILES: CCCc1nc2c(-c3ccc(F)cc3F)nc(N3C[C@H](C)O[C@@H](c4cnn(C5CC5)c4)C3)nc2nc1C] | I-1296 |
| 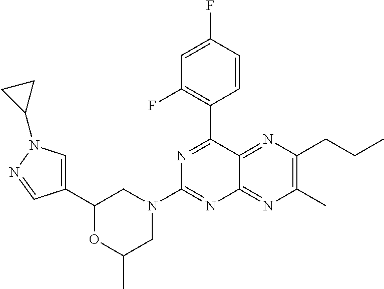 [SMILES: CCCc1nc2c(-c3ccc(F)cc3F)nc(N3CC(C)OC(c4cnn(C5CC5)c4)C3)nc2nc1C] | I-1297 |
| 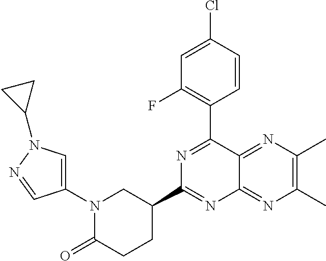 [SMILES: Cc1nc2nc([C@H]3CCC(=O)N(c4cnn(C5CC5)c4)C3)nc(-c3ccc(Cl)cc3F)c2nc1C] | I-1298 |
| 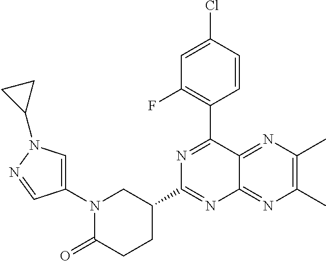 [SMILES: Cc1nc2nc([C@@H]3CCC(=O)N(c4cnn(C5CC5)c4)C3)nc(-c3ccc(Cl)cc3F)c2nc1C] | I-1299 |
| 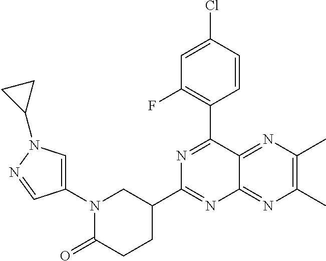 [SMILES: Cc1nc2nc(C3CCC(=O)N(c4cnn(C5CC5)c4)C3)nc(-c3ccc(Cl)cc3F)c2nc1C] | I-1300 |
| 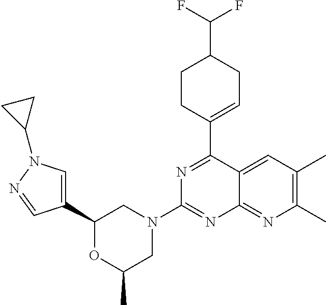 [SMILES: Cc1cc2c(C3=CCC(C(F)F)CC3)nc(N3C[C@@H](C)O[C@@H](c4cnn(C5CC5)c4)C3)nc2nc1C] | I-1301 |
| 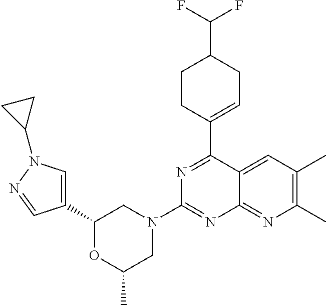 [SMILES: Cc1cc2c(C3=CCC(C(F)F)CC3)nc(N3C[C@@H](c4cnn(C5CC5)c4)O[C@@H](C)C3)nc2nc1C] | I-1302 |
| 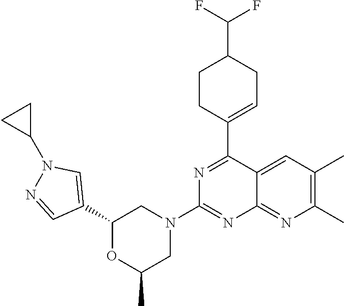 [SMILES: Cc1cc2c(C3=CCC(C(F)F)CC3)nc(N3C[C@@H](C)O[C@H](c4cnn(C5CC5)c4)C3)nc2nc1C] | I-1303 |
| 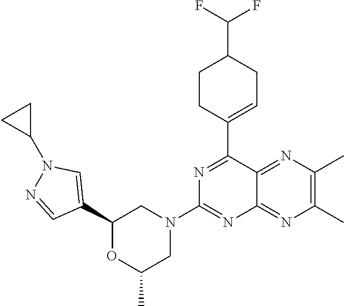 [SMILES: Cc1nc2nc(N3C[C@H](C)O[C@@H](c4cnn(C5CC5)c4)C3)nc(C3=CCC(C(F)F)CC3)c2nc1C] | I-1304 |
| 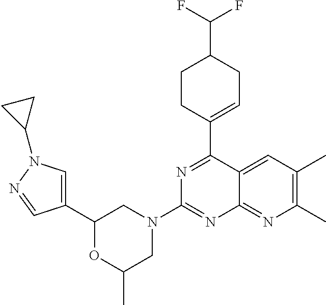 [SMILES: Cc1cc2c(C3=CCC(C(F)F)CC3)nc(N3CC(C)OC(c4cnn(C5CC5)c4)C3)nc2nc1C] | I-1305 |
| 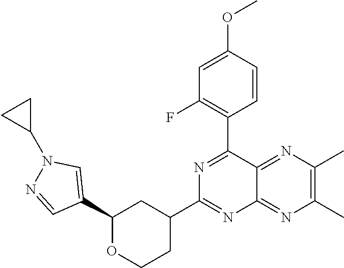 [SMILES: COc1ccc(-c2nc(C3CCO[C@@H](c4cnn(C5CC5)c4)C3)nc3nc(C)c(C)nc23)c(F)c1] | I-1306 |
| 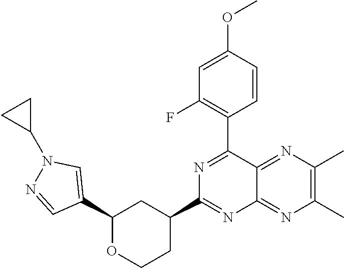 [SMILES: COc1ccc(-c2nc([C@H]3CCO[C@@H](c4cnn(C5CC5)c4)C3)nc3nc(C)c(C)nc23)c(F)c1] | I-1307 |
| 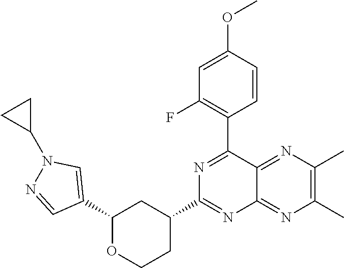 [SMILES: COc1ccc(-c2nc([C@@H]3CCO[C@H](c4cnn(C5CC5)c4)C3)nc3nc(C)c(C)nc23)c(F)c1] | I-1308 |
| 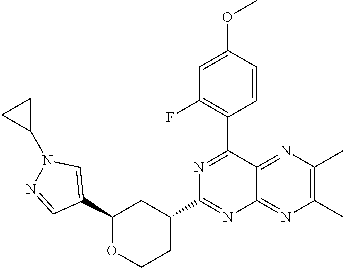 [SMILES: COc1ccc(-c2nc([C@@H]3CCO[C@@H](c4cnn(C5CC5)c4)C3)nc3nc(C)c(C)nc23)c(F)c1] | I-1309 |
| 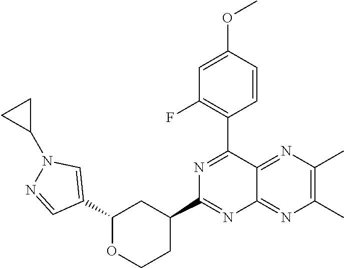 [SMILES: COc1ccc(-c2nc([C@H]3CCO[C@H](c4cnn(C5CC5)c4)C3)nc3nc(C)c(C)nc23)c(F)c1] | I-1310 |
| 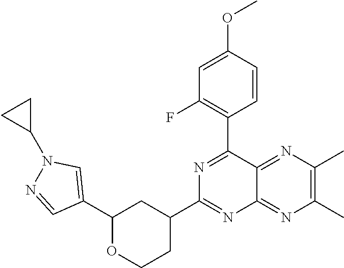 [SMILES: COc1ccc(-c2nc(C3CCOC(c4cnn(C5CC5)c4)C3)nc3nc(C)c(C)nc23)c(F)c1] | I-1311 |
| 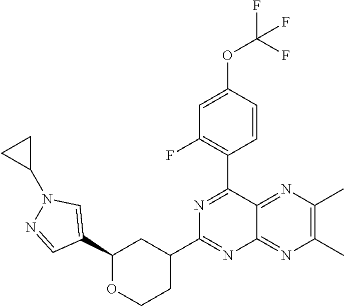 [SMILES: Cc1nc2nc(C3CCO[C@@H](c4cnn(C5CC5)c4)C3)nc(-c3ccc(OC(F)(F)F)cc3F)c2nc1C] | I-1312 |
| 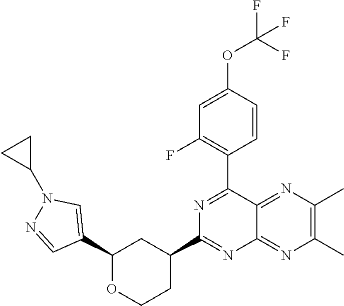 [SMILES: Cc1nc2nc([C@H]3CCO[C@@H](c4cnn(C5CC5)c4)C3)nc(-c3ccc(OC(F)(F)F)cc3F)c2nc1C] | I-1313 |
| 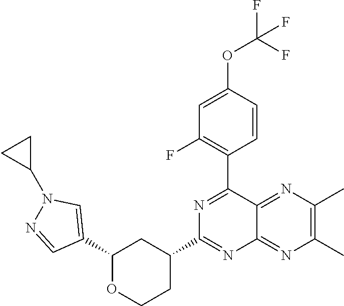 [SMILES: Cc1nc2nc([C@@H]3CCO[C@H](c4cnn(C5CC5)c4)C3)nc(-c3ccc(OC(F)(F)F)cc3F)c2nc1C] | I-1314 |
| 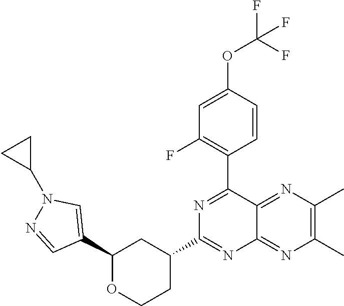 [SMILES: Cc1nc2nc([C@@H]3CCO[C@@H](c4cnn(C5CC5)c4)C3)nc(-c3ccc(OC(F)(F)F)cc3F)c2nc1C] | I-1315 |
| 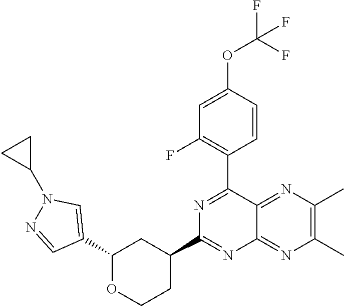 [SMILES: Cc1nc2nc([C@H]3CCO[C@H](c4cnn(C5CC5)c4)C3)nc(-c3ccc(OC(F)(F)F)cc3F)c2nc1C] | I-1316 |
| 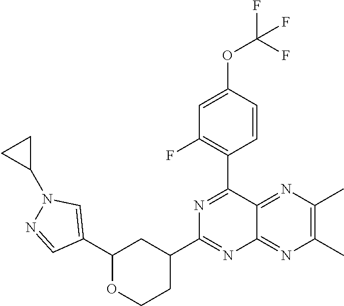 [SMILES: Cc1nc2nc(C3CCOC(c4cnn(C5CC5)c4)C3)nc(-c3ccc(OC(F)(F)F)cc3F)c2nc1C] | I-1317 |
| 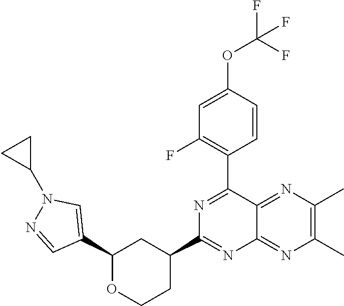 [SMILES: Cc1nc2nc([C@H]3CCO[C@@H](c4cnn(C5CC5)c4)C3)nc(-c3ccc(OC(F)(F)F)cc3F)c2nc1C] | I-1318 |
| 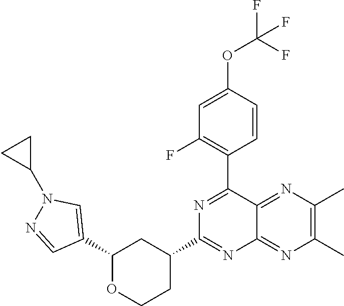 [SMILES: Cc1nc2nc([C@@H]3CCO[C@H](c4cnn(C5CC5)c4)C3)nc(-c3ccc(OC(F)(F)F)cc3F)c2nc1C] | I-1319 |
| 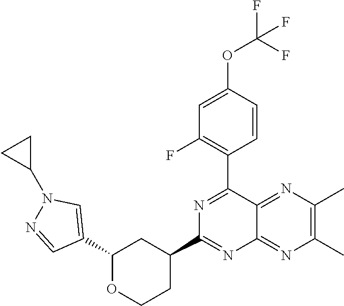 [SMILES: Cc1nc2nc([C@H]3CCO[C@H](c4cnn(C5CC5)c4)C3)nc(-c3ccc(OC(F)(F)F)cc3F)c2nc1C] | I-1320 |
| 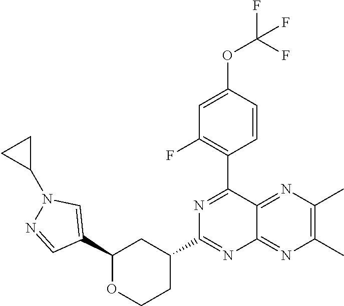 [SMILES: Cc1nc2nc([C@@H]3CCO[C@@H](c4cnn(C5CC5)c4)C3)nc(-c3ccc(OC(F)(F)F)cc3F)c2nc1C] | I-1321 |
| 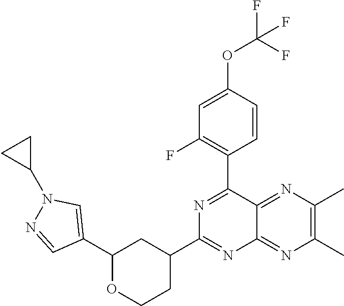 [SMILES: Cc1nc2nc(C3CCOC(c4cnn(C5CC5)c4)C3)nc(-c3ccc(OC(F)(F)F)cc3F)c2nc1C] | I-1322 |
| 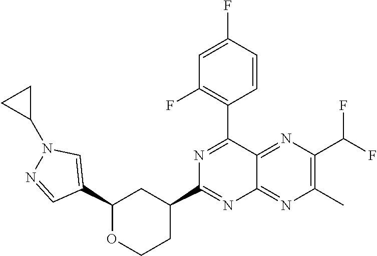 [SMILES: Cc1nc2nc([C@H]3CCO[C@@H](c4cnn(C5CC5)c4)C3)nc(-c3ccc(F)cc3F)c2nc1C(F)F] | I-1323 |
| 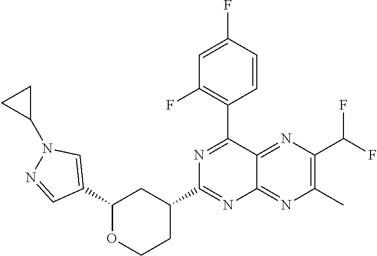 [SMILES: Cc1nc2nc([C@@H]3CCO[C@H](c4cnn(C5CC5)c4)C3)nc(-c3ccc(F)cc3F)c2nc1C(F)F] | I-1324 |
| 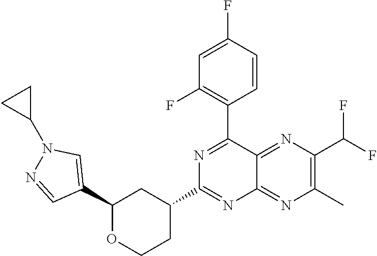 [SMILES: Cc1nc2nc([C@@H]3CCO[C@@H](c4cnn(C5CC5)c4)C3)nc(-c3ccc(F)cc3F)c2nc1C(F)F] | I-1325 |
| 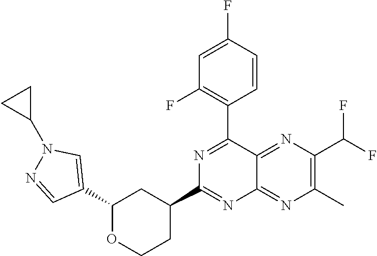 [SMILES: Cc1nc2nc([C@H]3CCO[C@H](c4cnn(C5CC5)c4)C3)nc(-c3ccc(F)cc3F)c2nc1C(F)F] | I-1326 |
| 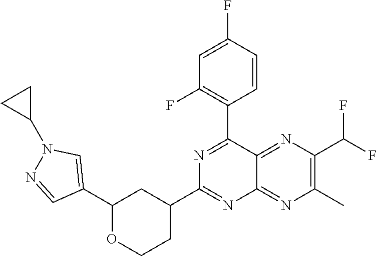 [SMILES: Cc1nc2nc(C3CCOC(c4cnn(C5CC5)c4)C3)nc(-c3ccc(F)cc3F)c2nc1C(F)F] | I-1327 |
| 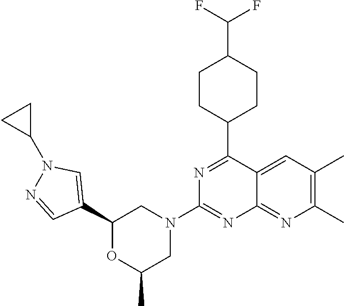 [SMILES: Cc1cc2c(C3CCC(C(F)F)CC3)nc(N3C[C@@H](C)O[C@@H](c4cnn(C5CC5)c4)C3)nc2nc1C] | I-1328 |
| 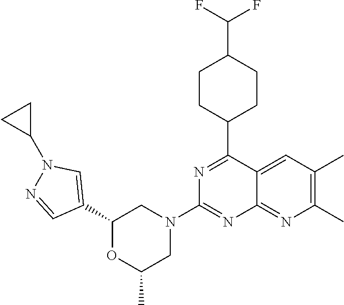 [SMILES: Cc1cc2c(C3CCC(C(F)F)CC3)nc(N3C[C@@H](c4cnn(C5CC5)c4)O[C@@H](C)C3)nc2nc1C] | I-1329 |
| 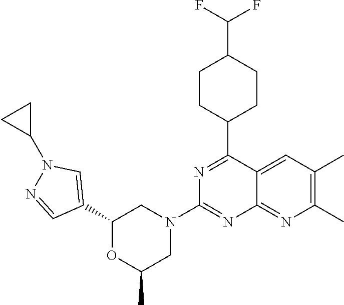 [SMILES: Cc1cc2c(C3CCC(C(F)F)CC3)nc(N3C[C@@H](C)O[C@H](c4cnn(C5CC5)c4)C3)nc2nc1C] | I-1330 |
| 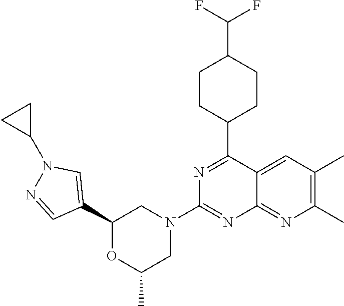 [SMILES: Cc1cc2c(C3CCC(C(F)F)CC3)nc(N3C[C@H](C)O[C@@H](c4cnn(C5CC5)c4)C3)nc2nc1C] | I-1331 |
| 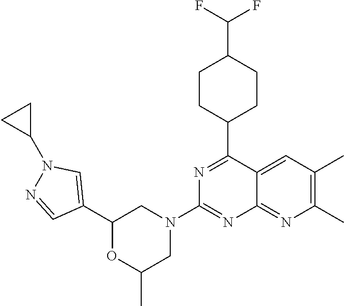 [SMILES: Cc1cc2c(C3CCC(C(F)F)CC3)nc(N3CC(C)OC(c4cnn(C5CC5)c4)C3)nc2nc1C] | I-1332 |
| 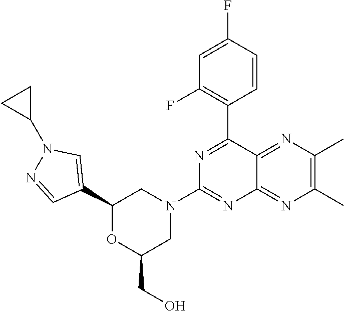 [SMILES: Cc1nc2nc(N3C[C@@H](CO)O[C@@H](c4cnn(C5CC5)c4)C3)nc(-c3ccc(F)cc3F)c2nc1C] | I-1333 |
| 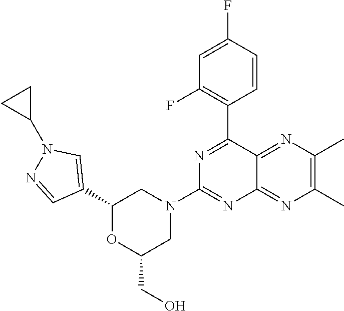 [SMILES: Cc1nc2nc(N3C[C@H](CO)O[C@H](c4cnn(C5CC5)c4)C3)nc(-c3ccc(F)cc3F)c2nc1C] | I-1334 |
| 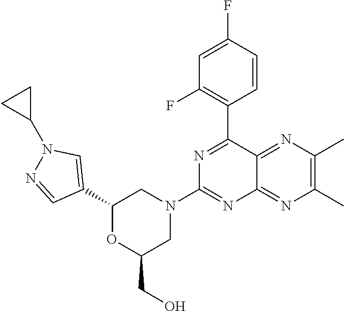 [SMILES: Cc1nc2nc(N3C[C@@H](CO)O[C@H](c4cnn(C5CC5)c4)C3)nc(-c3ccc(F)cc3F)c2nc1C] | I-1335 |
| 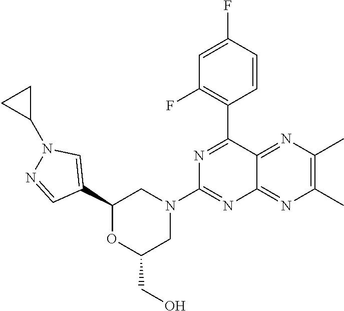 [SMILES: Cc1nc2nc(N3C[C@H](CO)O[C@@H](c4cnn(C5CC5)c4)C3)nc(-c3ccc(F)cc3F)c2nc1C] | I-1336 |
| 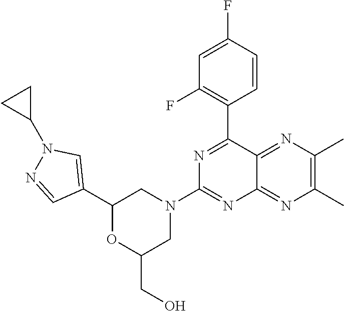 [SMILES: Cc1nc2nc(N3CC(CO)OC(c4cnn(C5CC5)c4)C3)nc(-c3ccc(F)cc3F)c2nc1C] | I-1337 |
| 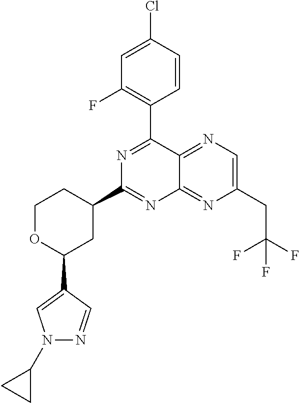 [SMILES: Fc1cc(Cl)ccc1-c1nc([C@@H]2CCO[C@H](c3cnn(C4CC4)c3)C2)nc2nc(CC(F)(F)F)cnc12] | I-1338 |
| 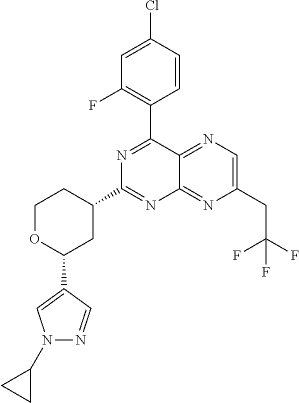 [SMILES: Fc1cc(Cl)ccc1-c1nc([C@H]2CCO[C@@H](c3cnn(C4CC4)c3)C2)nc2nc(CC(F)(F)F)cnc12] | I-1339 |
| 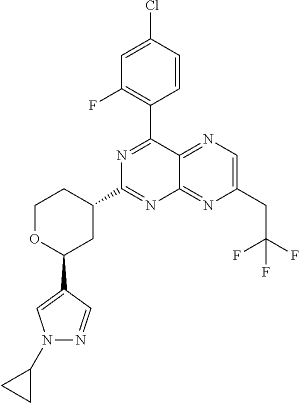 [SMILES: Fc1cc(Cl)ccc1-c1nc([C@H]2CCO[C@H](c3cnn(C4CC4)c3)C2)nc2nc(CC(F)(F)F)cnc12] | I-1340 |
| 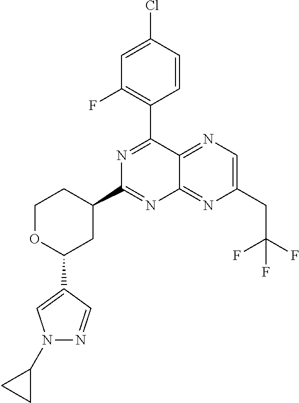 [SMILES: Fc1cc(Cl)ccc1-c1nc([C@@H]2CCO[C@@H](c3cnn(C4CC4)c3)C2)nc2nc(CC(F)(F)F)cnc12] | I-1341 |
| 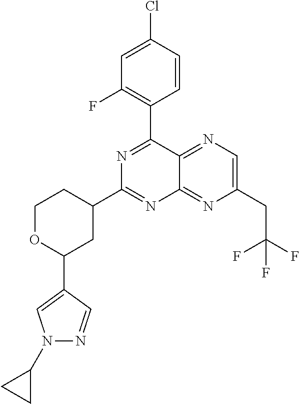 [SMILES: Fc1cc(Cl)ccc1-c1nc(C2CCOC(c3cnn(C4CC4)c3)C2)nc2nc(CC(F)(F)F)cnc12] | I-1342 |
| 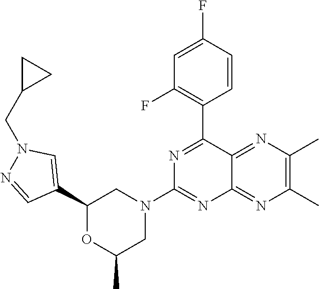 [SMILES: Cc1nc2nc(N3C[C@@H](C)O[C@@H](c4cnn(CC5CC5)c4)C3)nc(-c3ccc(F)cc3F)c2nc1C] | I-1343 |
| 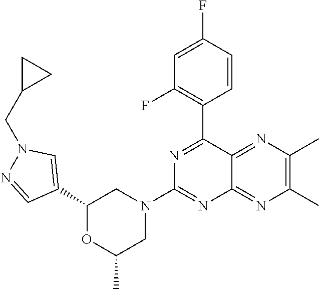 [SMILES: Cc1nc2nc(N3C[C@@H](c4cnn(CC5CC5)c4)O[C@@H](C)C3)nc(-c3ccc(F)cc3F)c2nc1C] | I-1344 |
| 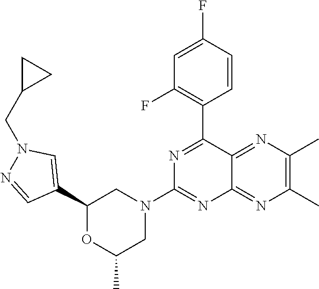 [SMILES: Cc1nc2nc(N3C[C@H](C)O[C@@H](c4cnn(CC5CC5)c4)C3)nc(-c3ccc(F)cc3F)c2nc1C] | I-1345 |
| 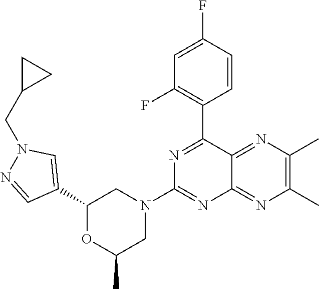 [SMILES: Cc1nc2nc(N3C[C@@H](C)O[C@H](c4cnn(CC5CC5)c4)C3)nc(-c3ccc(F)cc3F)c2nc1C] | I-1346 |
| 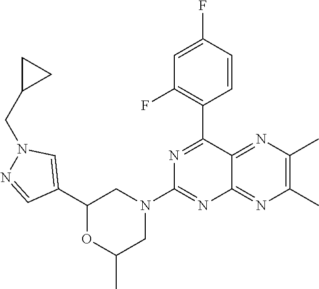 [SMILES: Cc1nc2nc(N3CC(C)OC(c4cnn(CC5CC5)c4)C3)nc(-c3ccc(F)cc3F)c2nc1C] | I-1347 |
| 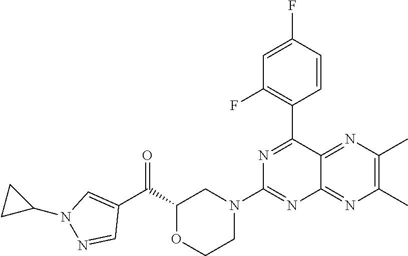 [SMILES: Cc1nc2nc(N3CCO[C@H](C(=O)c4cnn(C5CC5)c4)C3)nc(-c3ccc(F)cc3F)c2nc1C] | I-1348 |
| 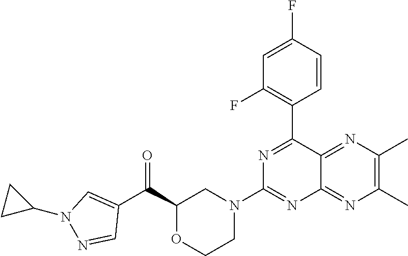 [SMILES: Cc1nc2nc(N3CCO[C@@H](C(=O)c4cnn(C5CC5)c4)C3)nc(-c3ccc(F)cc3F)c2nc1C] | I-1349 |
| 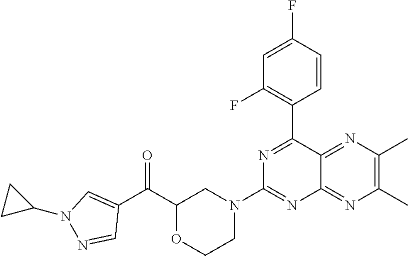 [SMILES: Cc1nc2nc(N3CCOC(C(=O)c4cnn(C5CC5)c4)C3)nc(-c3ccc(F)cc3F)c2nc1C] | I-1350 |
| 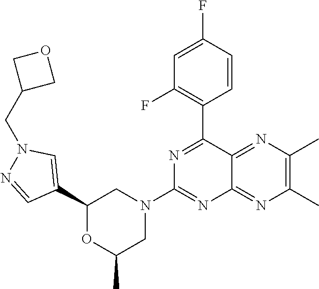 [SMILES: Cc1nc2nc(N3C[C@@H](C)O[C@@H](c4cnn(CC5COC5)c4)C3)nc(-c3ccc(F)cc3F)c2nc1C] | I-1351 |
| 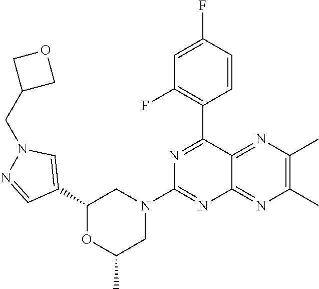 [SMILES: Cc1nc2nc(N3C[C@@H](c4cnn(CC5COC5)c4)O[C@@H](C)C3)nc(-c3ccc(F)cc3F)c2nc1C] | I-1352 |
| 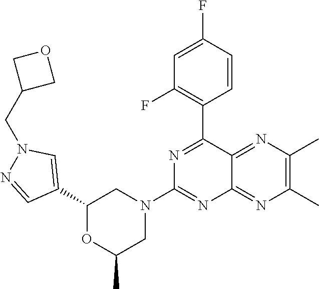 [SMILES: Cc1nc2nc(N3C[C@@H](C)O[C@H](c4cnn(CC5COC5)c4)C3)nc(-c3ccc(F)cc3F)c2nc1C] | I-1353 |
| 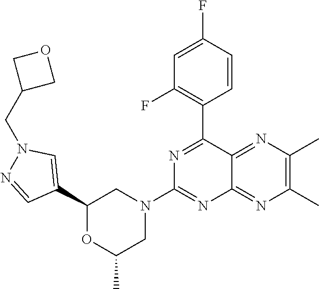 [SMILES: Cc1nc2nc(N3C[C@H](C)O[C@@H](c4cnn(CC5COC5)c4)C3)nc(-c3ccc(F)cc3F)c2nc1C] | I-1354 |
| 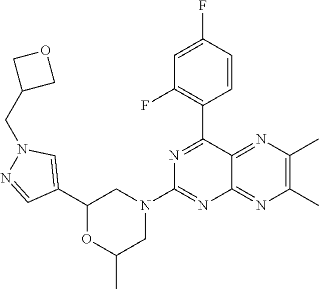 [SMILES: Cc1nc2nc(N3CC(C)OC(c4cnn(CC5COC5)c4)C3)nc(-c3ccc(F)cc3F)c2nc1C] | I-1355 |
| 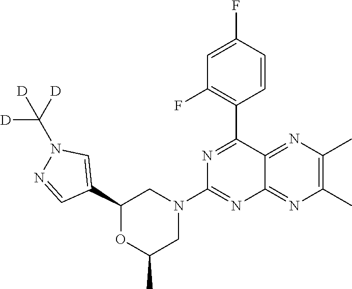 [SMILES: [2H]C([2H])([2H])n1cc([C@H]2CN(c3nc(-c4ccc(F)cc4F)c4nc(C)c(C)nc4n3)C[C@@H](C)O2)cn1] | I-1356 |
| 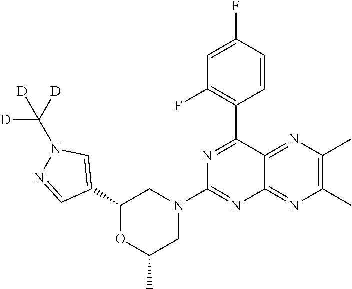 [SMILES: [2H]C([2H])([2H])n1cc([C@@H]2CN(c3nc(-c4ccc(F)cc4F)c4nc(C)c(C)nc4n3)C[C@H](C)O2)cn1] | I-1357 |
| 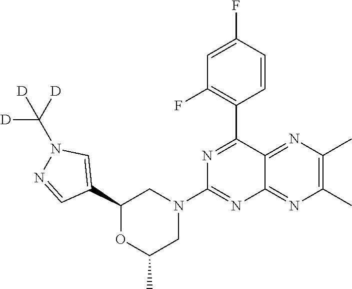 [SMILES: [2H]C([2H])([2H])n1cc([C@H]2CN(c3nc(-c4ccc(F)cc4F)c4nc(C)c(C)nc4n3)C[C@H](C)O2)cn1] | I-1358 |
| 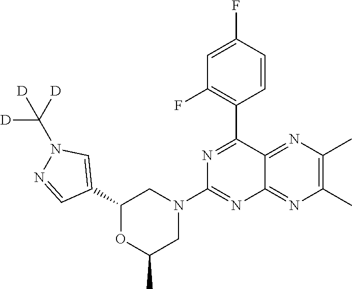 [SMILES: [2H]C([2H])([2H])n1cc([C@@H]2CN(c3nc(-c4ccc(F)cc4F)c4nc(C)c(C)nc4n3)C[C@@H](C)O2)cn1] | I-1359 |
| 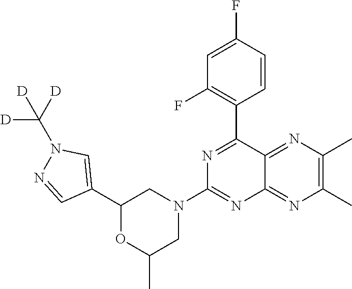 [SMILES: [2H]C([2H])([2H])n1cc(C2CN(c3nc(-c4ccc(F)cc4F)c4nc(C)c(C)nc4n3)CC(C)O2)cn1] | I-1360 |
| 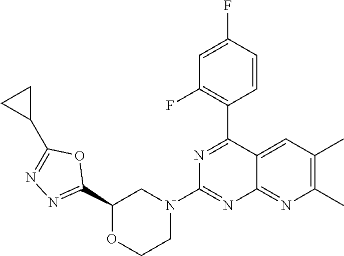 [SMILES: Cc1cc2c(-c3ccc(F)cc3F)nc(N3CCO[C@@H](c4nnc(C5CC5)o4)C3)nc2nc1C] | I-1361 |
| 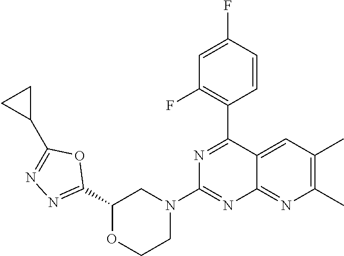 [SMILES: Cc1cc2c(-c3ccc(F)cc3F)nc(N3CCO[C@H](c4nnc(C5CC5)o4)C3)nc2nc1C] | I-1362 |
| 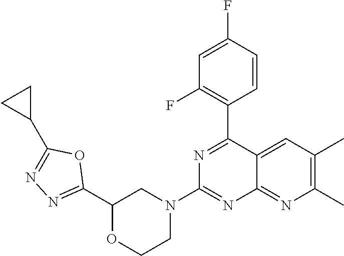 [SMILES: Cc1cc2c(-c3ccc(F)cc3F)nc(N3CCOC(c4nnc(C5CC5)o4)C3)nc2nc1C] | I-1363 |
| 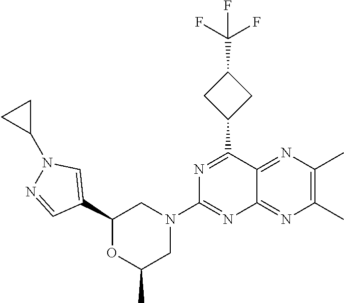 [SMILES: Cc1nc2nc(N3C[C@@H](C)O[C@@H](c4cnn(C5CC5)c4)C3)nc([C@H]3C[C@@H](C(F)(F)F)C3)c2nc1C] | I-1364 |
| 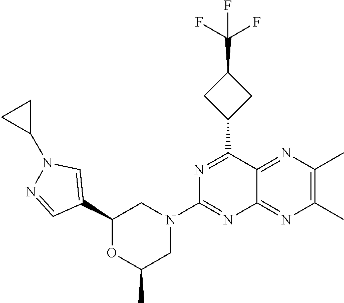 [SMILES: Cc1nc2nc(N3C[C@@H](C)O[C@@H](c4cnn(C5CC5)c4)C3)nc([C@H]3C[C@H](C(F)(F)F)C3)c2nc1C] | I-1365 |
| 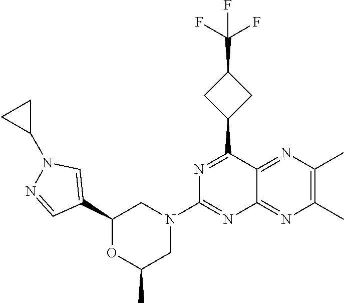 [SMILES: Cc1nc2nc(N3C[C@@H](C)O[C@@H](c4cnn(C5CC5)c4)C3)nc([C@H]3C[C@@H](C(F)(F)F)C3)c2nc1C] | I-1366 |
| 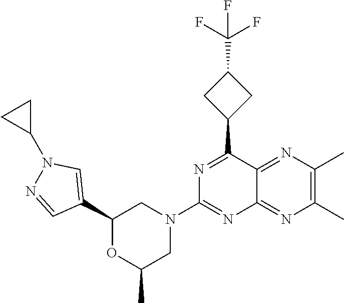 [SMILES: Cc1nc2nc(N3C[C@@H](C)O[C@@H](c4cnn(C5CC5)c4)C3)nc([C@H]3C[C@H](C(F)(F)F)C3)c2nc1C] | I-1367 |
| 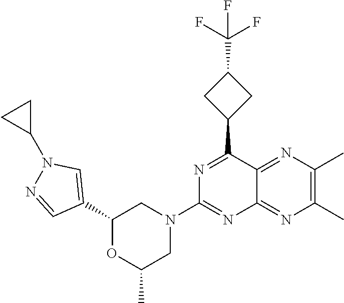 [SMILES: Cc1nc2nc(N3C[C@@H](c4cnn(C5CC5)c4)O[C@@H](C)C3)nc([C@H]3C[C@H](C(F)(F)F)C3)c2nc1C] | I-1368 |
| 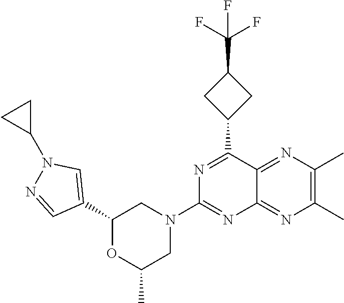 [SMILES: Cc1nc2nc(N3C[C@@H](c4cnn(C5CC5)c4)O[C@@H](C)C3)nc([C@H]3C[C@H](C(F)(F)F)C3)c2nc1C] | I-1369 |
| 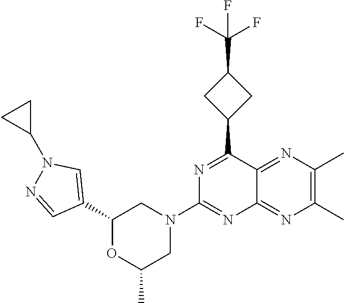 [SMILES: Cc1nc2nc(N3C[C@@H](c4cnn(C5CC5)c4)O[C@@H](C)C3)nc([C@H]3C[C@@H](C(F)(F)F)C3)c2nc1C] | I-1370 |
| 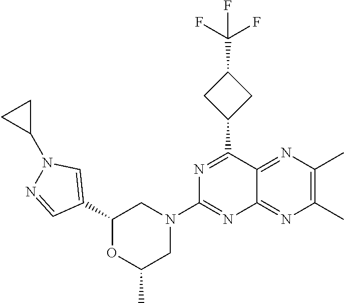 [SMILES: Cc1nc2nc(N3C[C@@H](c4cnn(C5CC5)c4)O[C@@H](C)C3)nc([C@H]3C[C@@H](C(F)(F)F)C3)c2nc1C] | I-1371 |
| 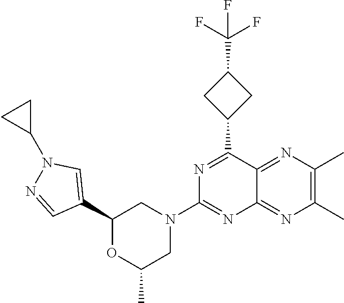 [SMILES: Cc1nc2nc(N3C[C@H](C)O[C@@H](c4cnn(C5CC5)c4)C3)nc([C@H]3C[C@@H](C(F)(F)F)C3)c2nc1C] | I-1372 |
| 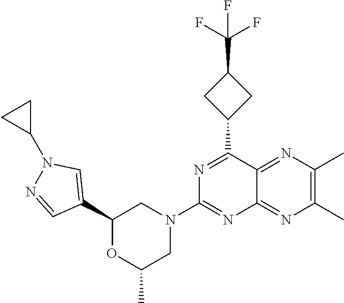 [SMILES: Cc1nc2nc(N3C[C@H](C)O[C@@H](c4cnn(C5CC5)c4)C3)nc([C@H]3C[C@H](C(F)(F)F)C3)c2nc1C] | I-1373 |
| 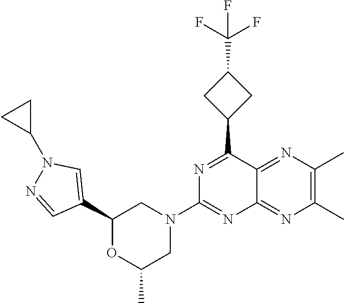 [SMILES: Cc1nc2nc(N3C[C@H](C)O[C@@H](c4cnn(C5CC5)c4)C3)nc([C@H]3C[C@H](C(F)(F)F)C3)c2nc1C] | I-1374 |
| 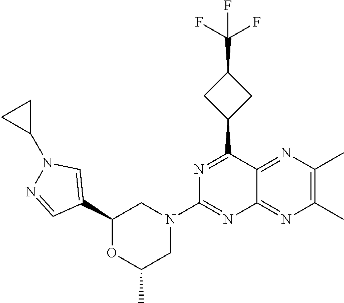 [SMILES: Cc1nc2nc(N3C[C@H](C)O[C@@H](c4cnn(C5CC5)c4)C3)nc([C@H]3C[C@@H](C(F)(F)F)C3)c2nc1C] | I-1375 |
| 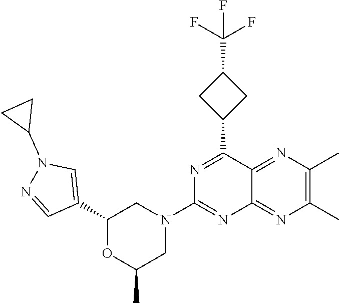 [SMILES: Cc1nc2nc(N3C[C@@H](C)O[C@H](c4cnn(C5CC5)c4)C3)nc([C@H]3C[C@@H](C(F)(F)F)C3)c2nc1C] | I-1376 |
| 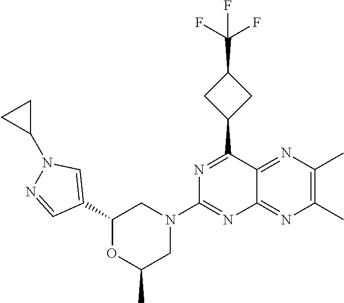 [SMILES: Cc1nc2nc(N3C[C@@H](C)O[C@H](c4cnn(C5CC5)c4)C3)nc([C@H]3C[C@@H](C(F)(F)F)C3)c2nc1C] | I-1377 |
| 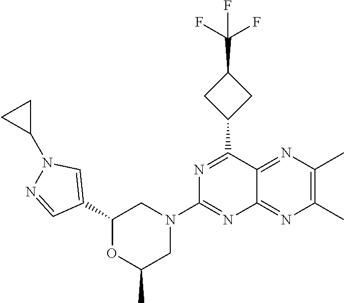 [SMILES: Cc1nc2nc(N3C[C@@H](C)O[C@H](c4cnn(C5CC5)c4)C3)nc([C@H]3C[C@H](C(F)(F)F)C3)c2nc1C] | I-1378 |
| 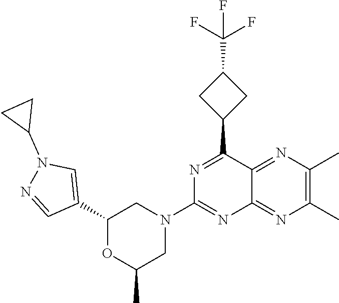 [SMILES: Cc1nc2nc(N3C[C@@H](C)O[C@H](c4cnn(C5CC5)c4)C3)nc([C@H]3C[C@H](C(F)(F)F)C3)c2nc1C] | I-1379 |
| 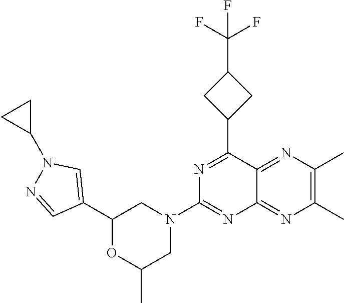 [SMILES: Cc1nc2nc(N3CC(C)OC(c4cnn(C5CC5)c4)C3)nc(C3CC(C(F)(F)F)C3)c2nc1C] | I-1380 |
| 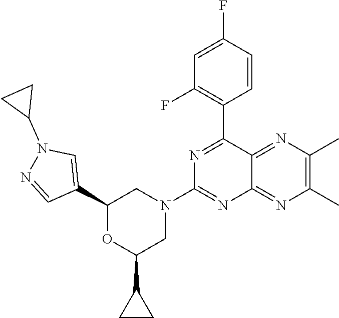 [SMILES: Cc1nc2nc(N3C[C@@H](C4CC4)O[C@@H](c4cnn(C5CC5)c4)C3)nc(-c3ccc(F)cc3F)c2nc1C] | I-1381 |
| 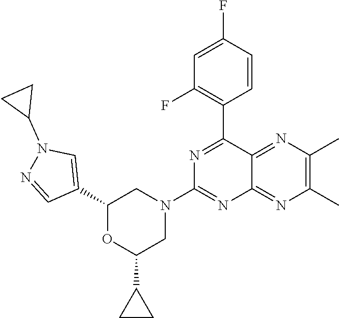 [SMILES: Cc1nc2nc(N3C[C@@H](c4cnn(C5CC5)c4)O[C@@H](C4CC4)C3)nc(-c3ccc(F)cc3F)c2nc1C] | I-1382 |
| 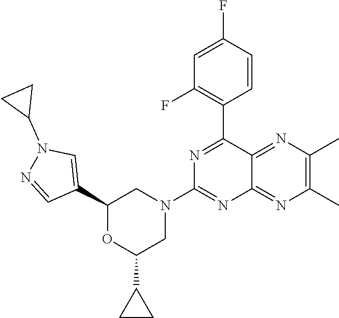 [SMILES: Cc1nc2nc(N3C[C@H](c4cnn(C5CC5)c4)O[C@@H](C4CC4)C3)nc(-c3ccc(F)cc3F)c2nc1C] | I-1383 |
| 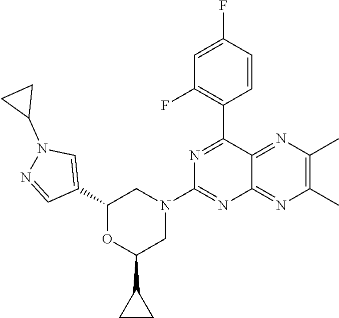 [SMILES: Cc1nc2nc(N3C[C@@H](c4cnn(C5CC5)c4)O[C@H](C4CC4)C3)nc(-c3ccc(F)cc3F)c2nc1C] | I-1384 |
| 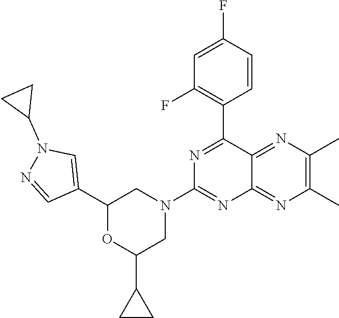 [SMILES: Cc1nc2nc(N3CC(c4cnn(C5CC5)c4)OC(C4CC4)C3)nc(-c3ccc(F)cc3F)c2nc1C] | I-1385 |
| 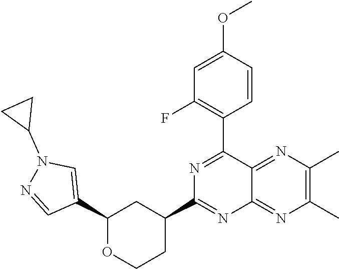 [SMILES: COc1ccc(-c2nc([C@H]3CCO[C@@H](c4cnn(C5CC5)c4)C3)nc3nc(C)c(C)nc23)c(F)c1] | I-1386 |
| 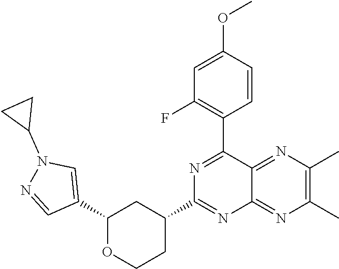 [SMILES: COc1ccc(-c2nc([C@@H]3CCO[C@H](c4cnn(C5CC5)c4)C3)nc3nc(C)c(C)nc23)c(F)c1] | I-1387 |
| 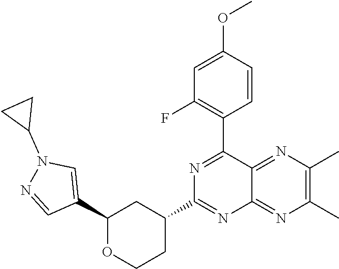 [SMILES: COc1ccc(-c2nc([C@@H]3CCO[C@@H](c4cnn(C5CC5)c4)C3)nc3nc(C)c(C)nc23)c(F)c1] | I-1388 |
| 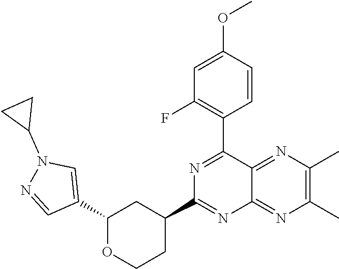 [SMILES: COc1ccc(-c2nc([C@H]3CCO[C@H](c4cnn(C5CC5)c4)C3)nc3nc(C)c(C)nc23)c(F)c1] | I-1389 |
| 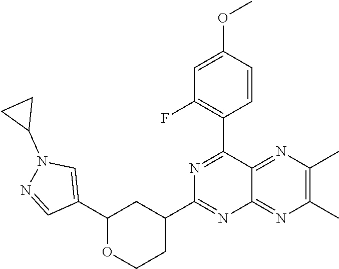 [SMILES: COc1ccc(-c2nc(C3CCOC(c4cnn(C5CC5)c4)C3)nc3nc(C)c(C)nc23)c(F)c1] | I-1390 |
| 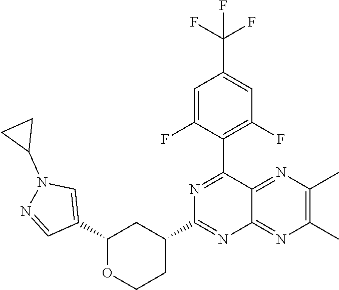 [SMILES: Cc1nc2nc([C@@H]3CCO[C@H](c4cnn(C5CC5)c4)C3)nc(-c3c(F)cc(C(F)(F)F)cc3F)c2nc1C] | I-1391 |
| 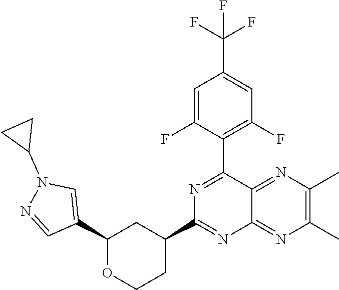 [SMILES: Cc1nc2nc([C@H]3CCO[C@@H](c4cnn(C5CC5)c4)C3)nc(-c3c(F)cc(C(F)(F)F)cc3F)c2nc1C] | I-1392 |
| 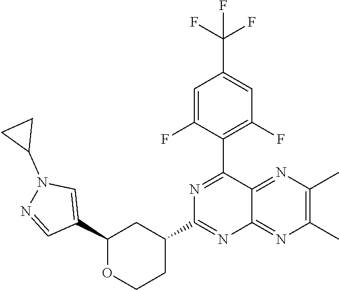 [SMILES: Cc1nc2nc([C@@H]3CCO[C@@H](c4cnn(C5CC5)c4)C3)nc(-c3c(F)cc(C(F)(F)F)cc3F)c2nc1C] | I-1393 |
| 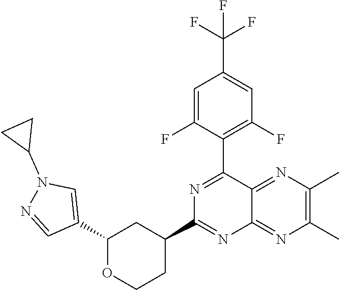 [SMILES: Cc1nc2nc([C@H]3CCO[C@H](c4cnn(C5CC5)c4)C3)nc(-c3c(F)cc(C(F)(F)F)cc3F)c2nc1C] | I-1394 |
| 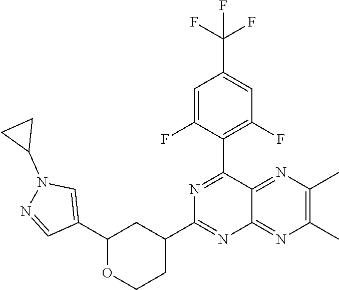 [SMILES: Cc1nc2nc(C3CCOC(c4cnn(C5CC5)c4)C3)nc(-c3c(F)cc(C(F)(F)F)cc3F)c2nc1C] | I-1395 |
| 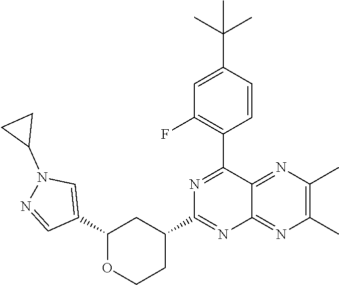 [SMILES: Cc1nc2nc([C@@H]3CCO[C@H](c4cnn(C5CC5)c4)C3)nc(-c3ccc(C(C)(C)C)cc3F)c2nc1C] | I-1396 |
| 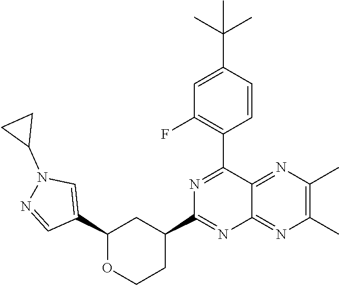 [SMILES: Cc1nc2nc([C@H]3CCO[C@@H](c4cnn(C5CC5)c4)C3)nc(-c3ccc(C(C)(C)C)cc3F)c2nc1C] | I-1397 |
| 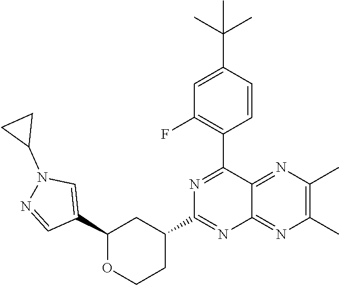 [SMILES: Cc1nc2nc([C@@H]3CCO[C@@H](c4cnn(C5CC5)c4)C3)nc(-c3ccc(C(C)(C)C)cc3F)c2nc1C] | I-1398 |
| 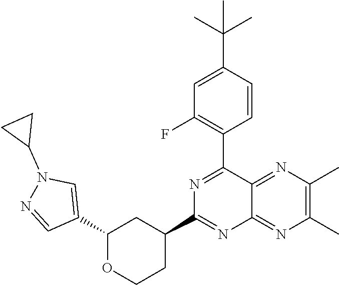 [SMILES: Cc1nc2nc([C@H]3CCO[C@H](c4cnn(C5CC5)c4)C3)nc(-c3ccc(C(C)(C)C)cc3F)c2nc1C] | I-1399 |
| 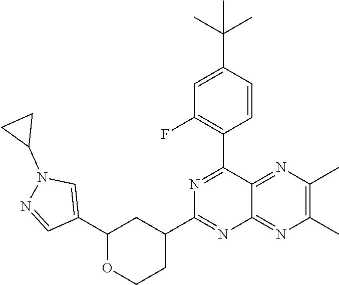 [SMILES: Cc1nc2nc(C3CCOC(c4cnn(C5CC5)c4)C3)nc(-c3ccc(C(C)(C)C)cc3F)c2nc1C] | I-1400 |
| 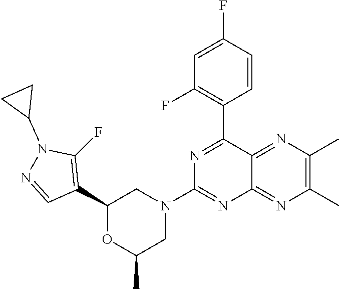 [SMILES: Cc1nc2nc(N3C[C@@H](C)O[C@@H](c4cnn(C5CC5)c4F)C3)nc(-c3ccc(F)cc3F)c2nc1C] | I-1401 |
| 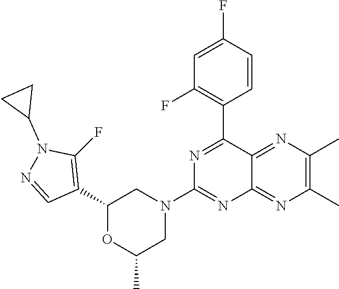 [SMILES: Cc1nc2nc(N3C[C@@H](c4cnn(C5CC5)c4F)O[C@@H](C)C3)nc(-c3ccc(F)cc3F)c2nc1C] | I-1402 |
| 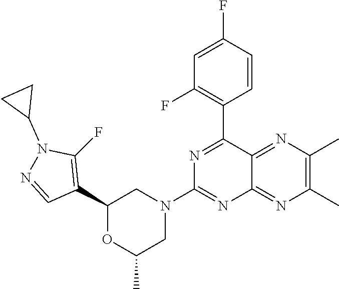 [SMILES: Cc1nc2nc(N3C[C@H](C)O[C@@H](c4cnn(C5CC5)c4F)C3)nc(-c3ccc(F)cc3F)c2nc1C] | I-1403 |
| 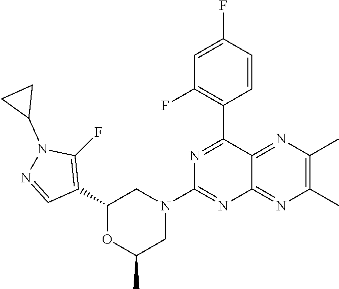 [SMILES: Cc1nc2nc(N3C[C@@H](C)O[C@H](c4cnn(C5CC5)c4F)C3)nc(-c3ccc(F)cc3F)c2nc1C] | I-1404 |
| 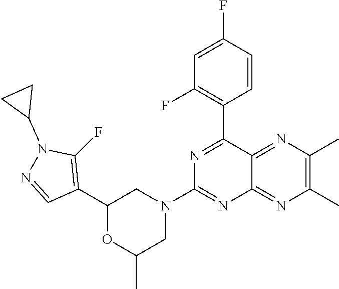 [SMILES: Cc1nc2nc(N3CC(C)OC(c4cnn(C5CC5)c4F)C3)nc(-c3ccc(F)cc3F)c2nc1C] | I-1405 |
| 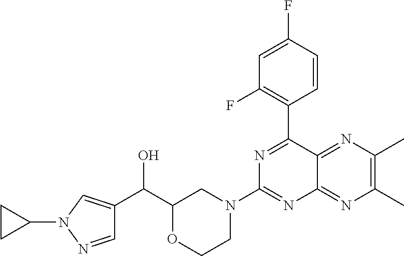 [SMILES: Cc1nc2nc(N3CCOC(C(O)c4cnn(C5CC5)c4)C3)nc(-c3ccc(F)cc3F)c2nc1C] | I-1406 |
| 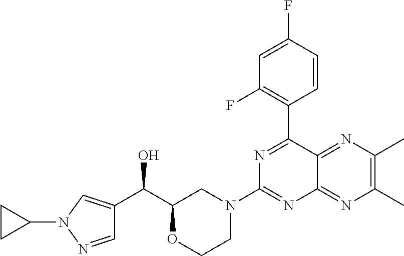 [SMILES: Cc1nc2nc(N3CCO[C@@H]([C@H](O)c4cnn(C5CC5)c4)C3)nc(-c3ccc(F)cc3F)c2nc1C] | I-1407 |
| 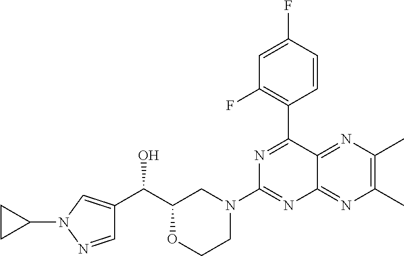 [SMILES: Cc1nc2nc(N3CCO[C@H]([C@@H](O)c4cnn(C5CC5)c4)C3)nc(-c3ccc(F)cc3F)c2nc1C] | I-1408 |
| 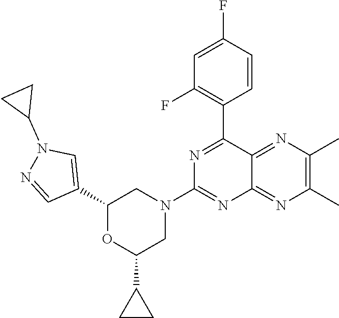 [SMILES: Cc1nc2nc(N3C[C@@H](c4cnn(C5CC5)c4)O[C@@H](C4CC4)C3)nc(-c3ccc(F)cc3F)c2nc1C] | I-1409 |
| 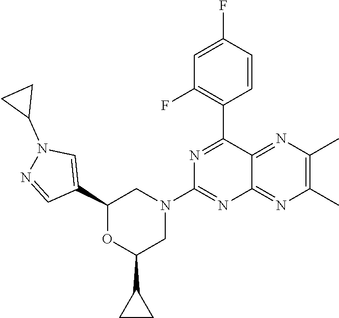 [SMILES: Cc1nc2nc(N3C[C@@H](C4CC4)O[C@@H](c4cnn(C5CC5)c4)C3)nc(-c3ccc(F)cc3F)c2nc1C] | I-1410 |
| 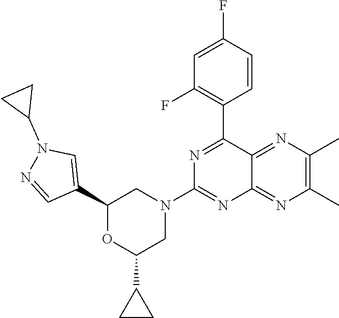 [SMILES: Cc1nc2nc(N3C[C@H](c4cnn(C5CC5)c4)O[C@@H](C4CC4)C3)nc(-c3ccc(F)cc3F)c2nc1C] | I-1411 |
| 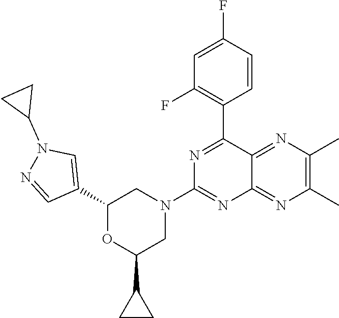 [SMILES: Cc1nc2nc(N3C[C@@H](c4cnn(C5CC5)c4)O[C@H](C4CC4)C3)nc(-c3ccc(F)cc3F)c2nc1C] | I-1412 |
| 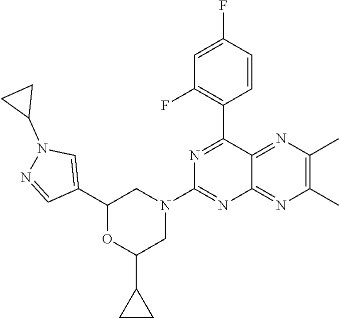 [SMILES: Cc1nc2nc(N3CC(c4cnn(C5CC5)c4)OC(C4CC4)C3)nc(-c3ccc(F)cc3F)c2nc1C] | I-1413 |
| 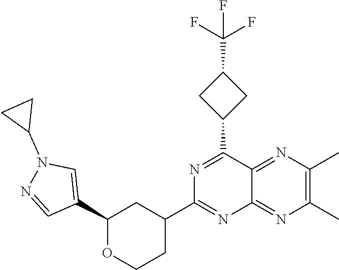 [SMILES: Cc1nc2nc(C3CCO[C@@H](c4cnn(C5CC5)c4)C3)nc([C@H]3C[C@@H](C(F)(F)F)C3)c2nc1C] | I-1414 |
| 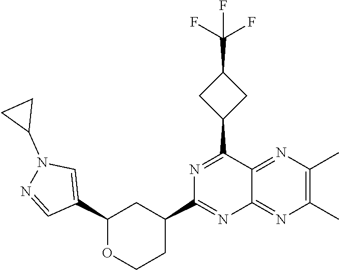 [SMILES: Cc1nc2nc([C@H]3CCO[C@@H](c4cnn(C5CC5)c4)C3)nc([C@H]3C[C@@H](C(F)(F)F)C3)c2nc1C] | I-1415 |
| 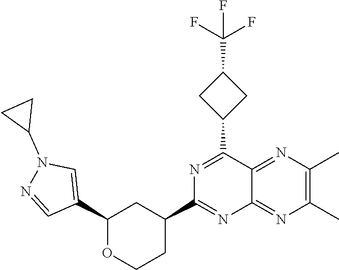 [SMILES: Cc1nc2nc([C@H]3CCO[C@@H](c4cnn(C5CC5)c4)C3)nc([C@H]3C[C@@H](C(F)(F)F)C3)c2nc1C] | I-1416 |
| 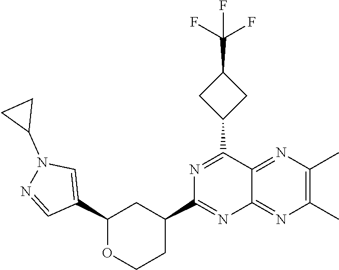 [SMILES: Cc1nc2nc([C@H]3CCO[C@@H](c4cnn(C5CC5)c4)C3)nc([C@H]3C[C@H](C(F)(F)F)C3)c2nc1C] | I-1417 |
| 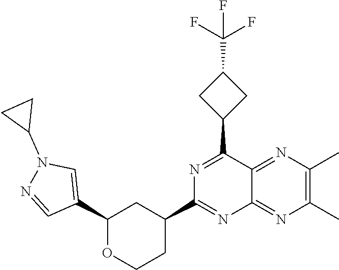 [SMILES: Cc1nc2nc([C@H]3CCO[C@@H](c4cnn(C5CC5)c4)C3)nc([C@H]3C[C@H](C(F)(F)F)C3)c2nc1C] | I-1418 |
| 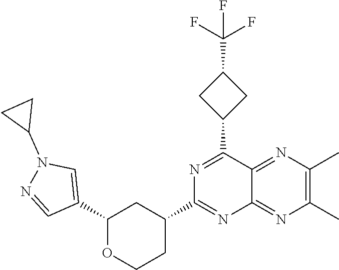 [SMILES: Cc1nc2nc([C@@H]3CCO[C@H](c4cnn(C5CC5)c4)C3)nc([C@H]3C[C@@H](C(F)(F)F)C3)c2nc1C] | I-1419 |
| 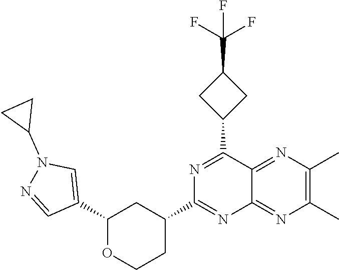 [SMILES: Cc1nc2nc([C@@H]3CCO[C@H](c4cnn(C5CC5)c4)C3)nc([C@H]3C[C@H](C(F)(F)F)C3)c2nc1C] | I-1420 |
| 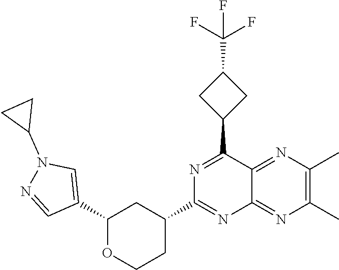 [SMILES: Cc1nc2nc([C@@H]3CCO[C@H](c4cnn(C5CC5)c4)C3)nc([C@H]3C[C@H](C(F)(F)F)C3)c2nc1C] | I-1421 |
| 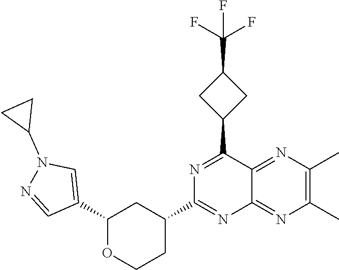 [SMILES: Cc1nc2nc([C@@H]3CCO[C@H](c4cnn(C5CC5)c4)C3)nc([C@H]3C[C@@H](C(F)(F)F)C3)c2nc1C] | I-1422 |
| 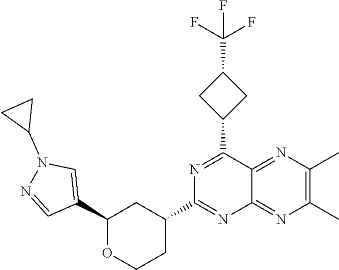 [SMILES: Cc1nc2nc([C@@H]3CCO[C@@H](c4cnn(C5CC5)c4)C3)nc([C@H]3C[C@@H](C(F)(F)F)C3)c2nc1C] | I-1423 |
| 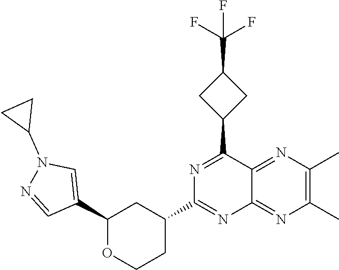 [SMILES: Cc1nc2nc([C@@H]3CCO[C@@H](c4cnn(C5CC5)c4)C3)nc([C@H]3C[C@@H](C(F)(F)F)C3)c2nc1C] | I-1424 |
| 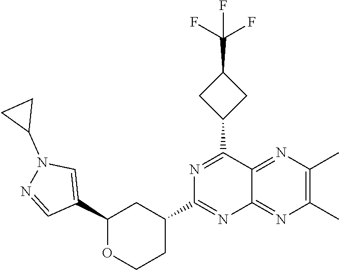 [SMILES: Cc1nc2nc([C@@H]3CCO[C@@H](c4cnn(C5CC5)c4)C3)nc([C@H]3C[C@H](C(F)(F)F)C3)c2nc1C] | I-1425 |
| 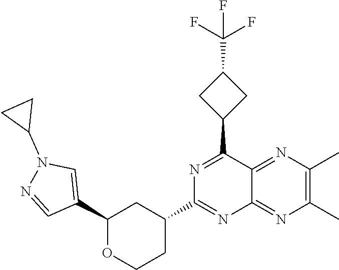 [SMILES: Cc1nc2nc([C@@H]3CCO[C@@H](c4cnn(C5CC5)c4)C3)nc([C@H]3C[C@H](C(F)(F)F)C3)c2nc1C] | I-1426 |
| 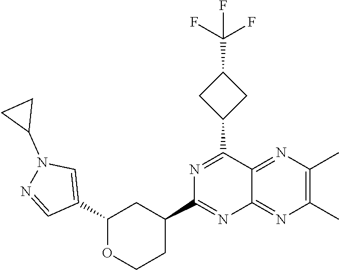 [SMILES: Cc1nc2nc([C@H]3CCO[C@H](c4cnn(C5CC5)c4)C3)nc([C@H]3C[C@@H](C(F)(F)F)C3)c2nc1C] | I-1427 |
| 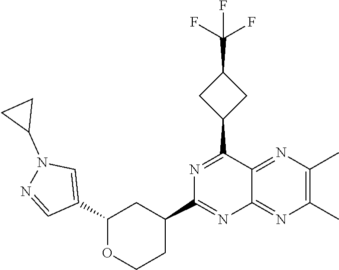 [SMILES: Cc1nc2nc([C@H]3CCO[C@H](c4cnn(C5CC5)c4)C3)nc([C@H]3C[C@@H](C(F)(F)F)C3)c2nc1C] | I-1428 |
| 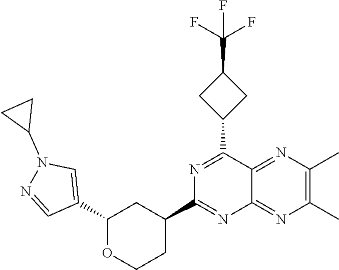 [SMILES: Cc1nc2nc([C@H]3CCO[C@H](c4cnn(C5CC5)c4)C3)nc([C@H]3C[C@H](C(F)(F)F)C3)c2nc1C] | I-1429 |
| 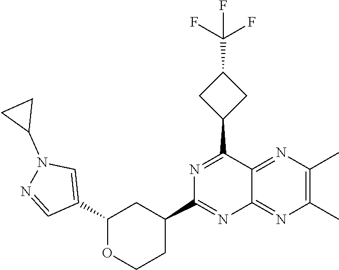 [SMILES: Cc1nc2nc([C@H]3CCO[C@H](c4cnn(C5CC5)c4)C3)nc([C@H]3C[C@H](C(F)(F)F)C3)c2nc1C] | I-1430 |
| 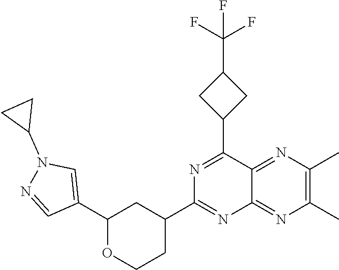 [SMILES: Cc1nc2nc(C3CCOC(c4cnn(C5CC5)c4)C3)nc(C3CC(C(F)(F)F)C3)c2nc1C] | I-1431 |
| 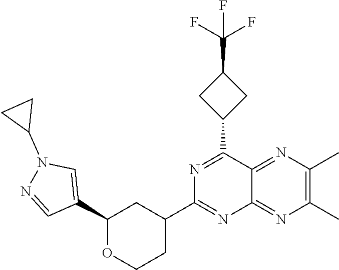 [SMILES: Cc1nc2nc(C3CCO[C@@H](c4cnn(C5CC5)c4)C3)nc([C@H]3C[C@H](C(F)(F)F)C3)c2nc1C] | I-1432 |
| 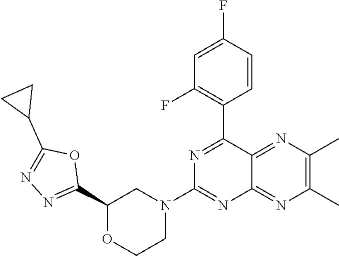 [SMILES: Cc1nc2nc(N3CCO[C@@H](c4nnc(C5CC5)o4)C3)nc(-c3ccc(F)cc3F)c2nc1C] | I-1433 |
| 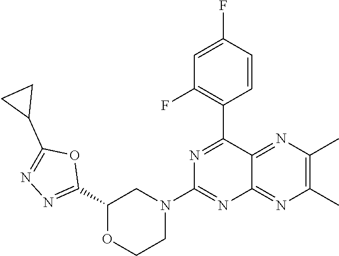 [SMILES: Cc1nc2nc(N3CCO[C@H](c4nnc(C5CC5)o4)C3)nc(-c3ccc(F)cc3F)c2nc1C] | I-1434 |
| 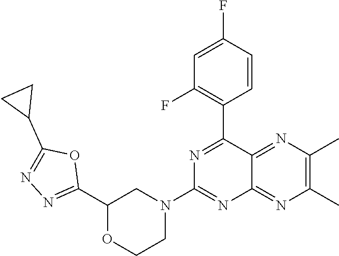 [SMILES: Cc1nc2nc(N3CCOC(c4nnc(C5CC5)o4)C3)nc(-c3ccc(F)cc3F)c2nc1C] | I-1435 |
| 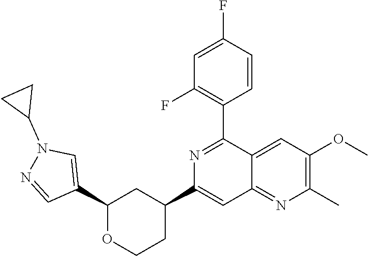 [SMILES: COc1cc2c(-c3ccc(F)cc3F)nc([C@H]3CCO[C@@H](c4cnn(C5CC5)c4)C3)cc2nc1C] | I-1436 |
| 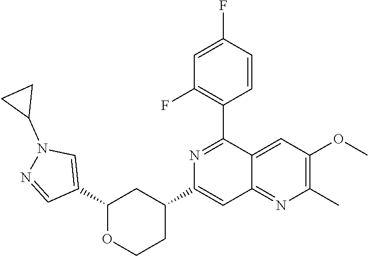 [SMILES: COc1cc2c(-c3ccc(F)cc3F)nc([C@@H]3CCO[C@H](c4cnn(C5CC5)c4)C3)cc2nc1C] | I-1437 |
| 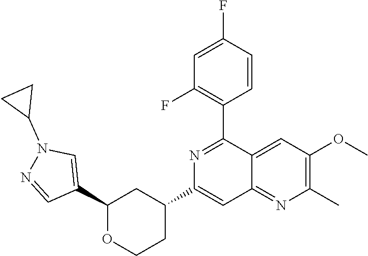 [SMILES: COc1cc2c(-c3ccc(F)cc3F)nc([C@@H]3CCO[C@@H](c4cnn(C5CC5)c4)C3)cc2nc1C] | I-1438 |
| 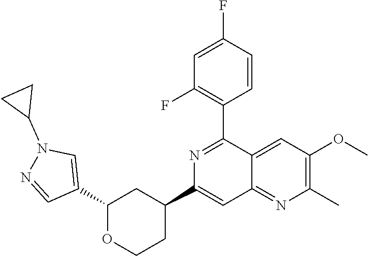 [SMILES: COc1cc2c(-c3ccc(F)cc3F)nc([C@H]3CCO[C@H](c4cnn(C5CC5)c4)C3)cc2nc1C] | I-1439 |
| 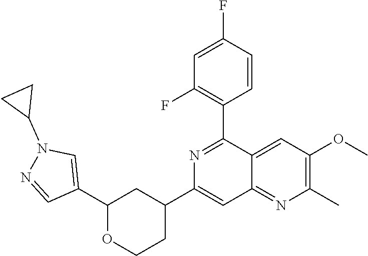 [SMILES: COc1cc2c(-c3ccc(F)cc3F)nc(C3CCOC(c4cnn(C5CC5)c4)C3)cc2nc1C] | I-1440 |
| 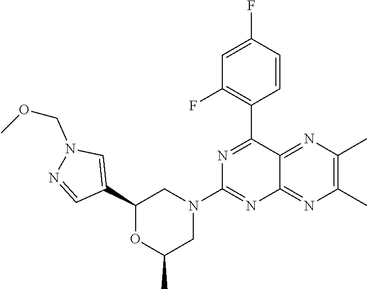 | I-1441 |
| 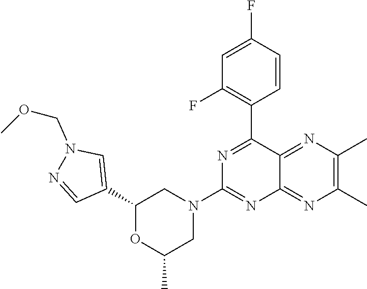 | I-1442 |
| 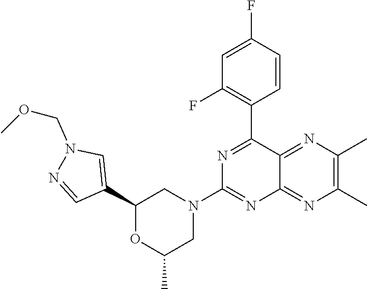 | I-1443 |
| 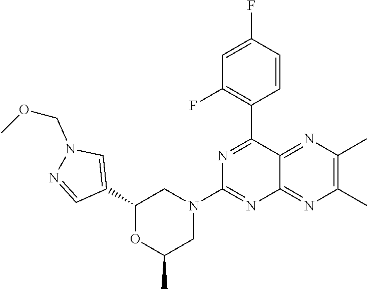 | I-1444 |
| 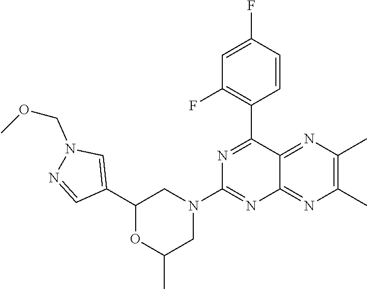 | I-1445 |
| 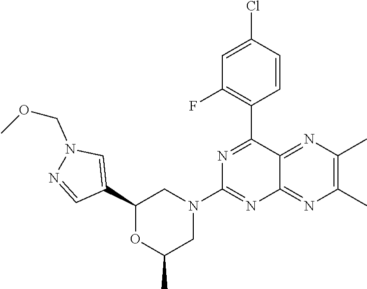 | I-1446 |
| 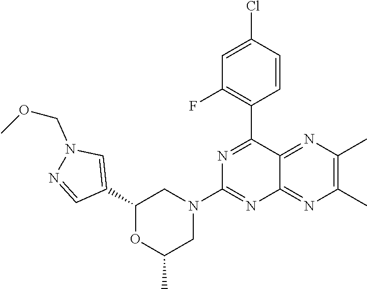 | I-1447 |
| 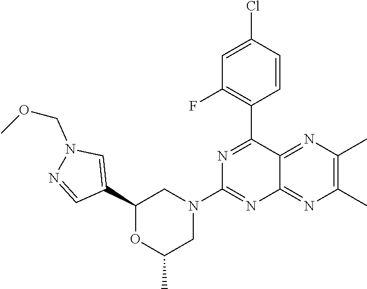 | I-1448 |
| 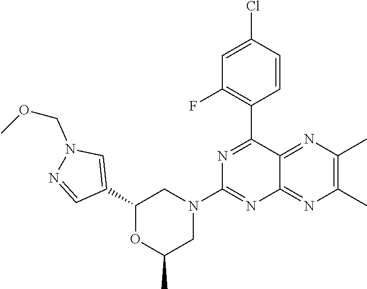 | I-1449 |
| 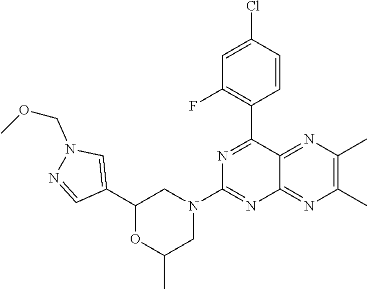 | I-1450 |
| 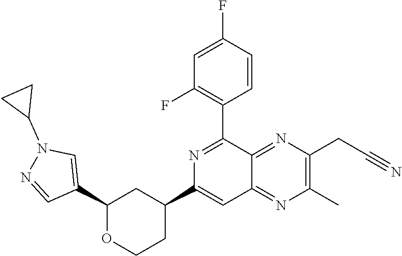 [SMILES: Cc1nc2cc([C@H]3CCO[C@@H](c4cnn(C5CC5)c4)C3)nc(-c3ccc(F)cc3F)c2nc1CC#N] | I-1451 |
| 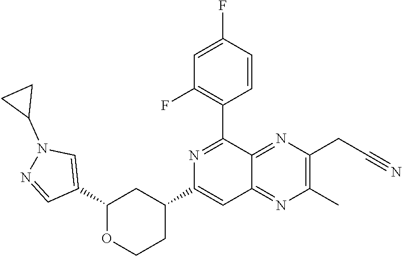 [SMILES: Cc1nc2cc([C@@H]3CCO[C@H](c4cnn(C5CC5)c4)C3)nc(-c3ccc(F)cc3F)c2nc1CC#N] | I-1452 |
| 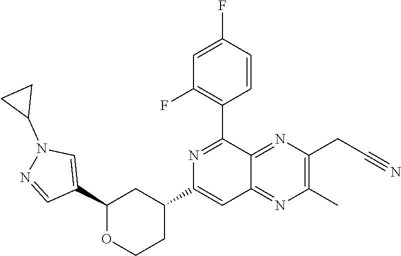 [SMILES: Cc1nc2cc([C@@H]3CCO[C@@H](c4cnn(C5CC5)c4)C3)nc(-c3ccc(F)cc3F)c2nc1CC#N] | I-1453 |
| 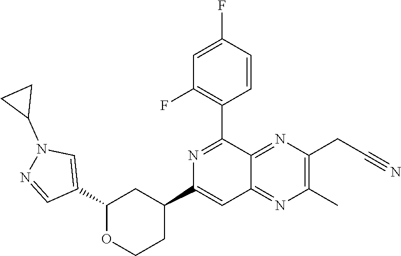 [SMILES: Cc1nc2cc([C@H]3CCO[C@H](c4cnn(C5CC5)c4)C3)nc(-c3ccc(F)cc3F)c2nc1CC#N] | I-1454 |
| 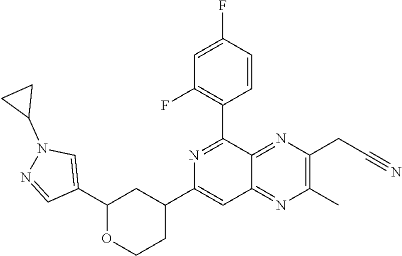 [SMILES: Cc1nc2cc(C3CCOC(c4cnn(C5CC5)c4)C3)nc(-c3ccc(F)cc3F)c2nc1CC#N] | I-1455 |
| 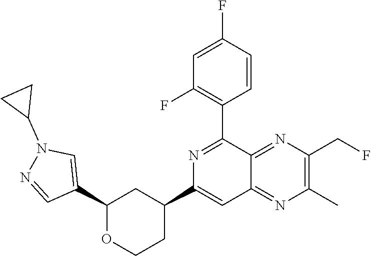 [SMILES: Cc1nc2cc([C@H]3CCO[C@@H](c4cnn(C5CC5)c4)C3)nc(-c3ccc(F)cc3F)c2nc1CF] | I-1456 |
| 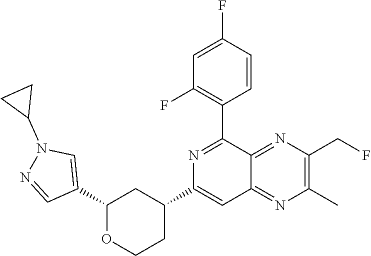 [SMILES: Cc1nc2cc([C@@H]3CCO[C@H](c4cnn(C5CC5)c4)C3)nc(-c3ccc(F)cc3F)c2nc1CF] | I-1457 |
| 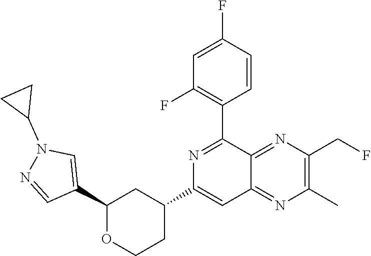 [SMILES: Cc1nc2cc([C@@H]3CCO[C@@H](c4cnn(C5CC5)c4)C3)nc(-c3ccc(F)cc3F)c2nc1CF] | I-1458 |
| 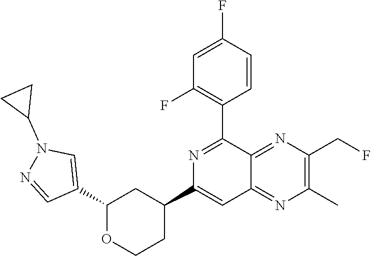 [SMILES: Cc1nc2cc([C@H]3CCO[C@H](c4cnn(C5CC5)c4)C3)nc(-c3ccc(F)cc3F)c2nc1CF] | I-1459 |
| 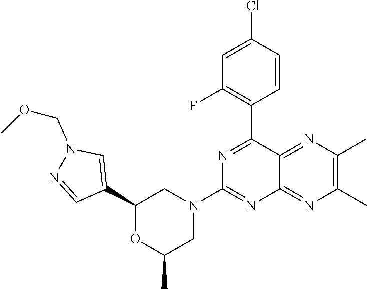 | I-1460 |
| 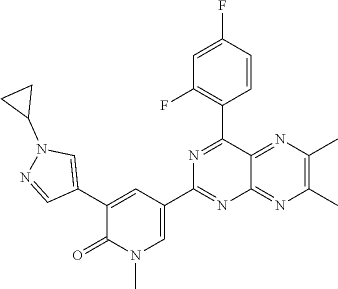 [SMILES: Cc1nc2nc(-c3cc(-c4cnn(C5CC5)c4)c(=O)n(C)c3)nc(-c3ccc(F)cc3F)c2nc1C] | I-1461 |
| 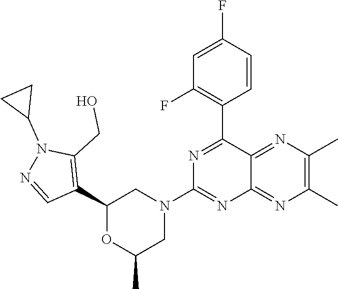 [SMILES: Cc1nc2nc(N3C[C@@H](C)O[C@@H](c4cnn(C5CC5)c4CO)C3)nc(-c3ccc(F)cc3F)c2nc1C] | I-1462 |
| 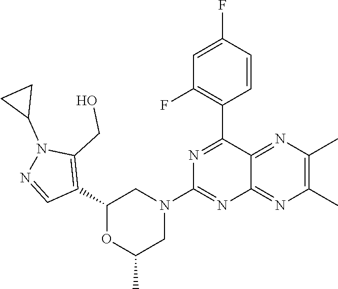 [SMILES: Cc1nc2nc(N3C[C@@H](c4cnn(C5CC5)c4CO)O[C@@H](C)C3)nc(-c3ccc(F)cc3F)c2nc1C] | I-1463 |
| 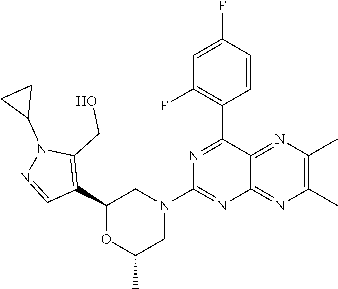 [SMILES: Cc1nc2nc(N3C[C@H](C)O[C@@H](c4cnn(C5CC5)c4CO)C3)nc(-c3ccc(F)cc3F)c2nc1C] | I-1464 |
| 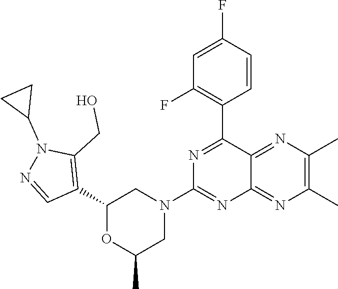 [SMILES: Cc1nc2nc(N3C[C@@H](C)O[C@H](c4cnn(C5CC5)c4CO)C3)nc(-c3ccc(F)cc3F)c2nc1C] | I-1465 |
| 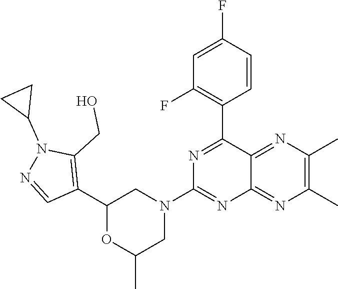 [SMILES: Cc1nc2nc(N3CC(C)OC(c4cnn(C5CC5)c4CO)C3)nc(-c3ccc(F)cc3F)c2nc1C] | I-1466 |
| 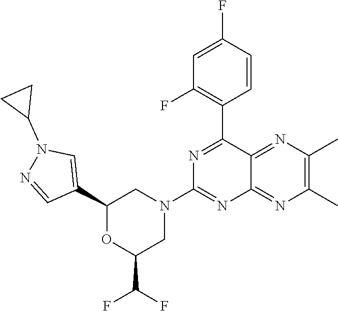 [SMILES: Cc1nc2nc(N3C[C@@H](C(F)F)O[C@@H](c4cnn(C5CC5)c4)C3)nc(-c3ccc(F)cc3F)c2nc1C] | I-1467 |
| 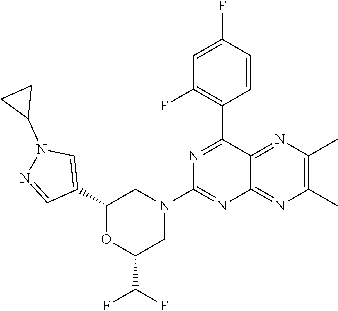 [SMILES: Cc1nc2nc(N3C[C@@H](c4cnn(C5CC5)c4)O[C@@H](C(F)F)C3)nc(-c3ccc(F)cc3F)c2nc1C] | I-1468 |
| 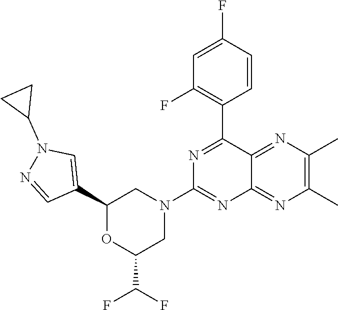 [SMILES: Cc1nc2nc(N3C[C@H](c4cnn(C5CC5)c4)O[C@@H](C(F)F)C3)nc(-c3ccc(F)cc3F)c2nc1C] | I-1469 |
| 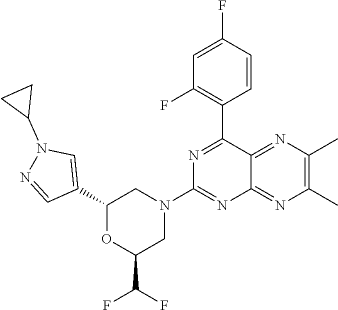 [SMILES: Cc1nc2nc(N3C[C@@H](c4cnn(C5CC5)c4)O[C@H](C(F)F)C3)nc(-c3ccc(F)cc3F)c2nc1C] | I-1470 |
| 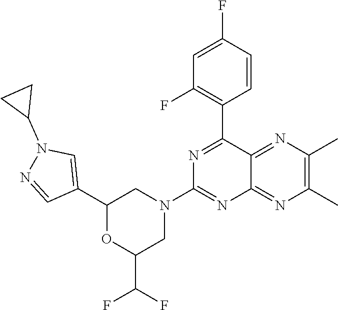 [SMILES: Cc1nc2nc(N3CC(c4cnn(C5CC5)c4)OC(C(F)F)C3)nc(-c3ccc(F)cc3F)c2nc1C] | I-1471 |
| 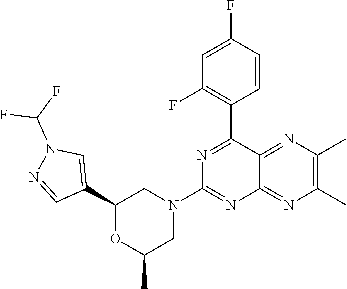 [SMILES: Cc1nc2nc(N3C[C@@H](C)O[C@@H](c4cnn(C(F)F)c4)C3)nc(-c3ccc(F)cc3F)c2nc1C] | I-1472 |
| 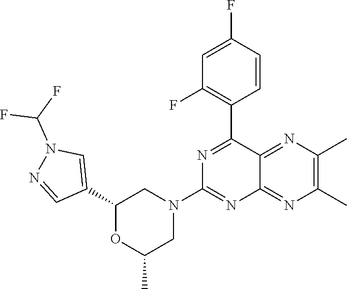 [SMILES: Cc1nc2nc(N3C[C@@H](c4cnn(C(F)F)c4)O[C@@H](C)C3)nc(-c3ccc(F)cc3F)c2nc1C] | I-1473 |
| 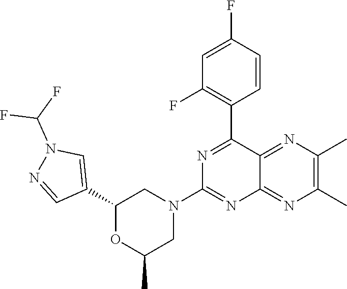 [SMILES: Cc1nc2nc(N3C[C@@H](C)O[C@H](c4cnn(C(F)F)c4)C3)nc(-c3ccc(F)cc3F)c2nc1C] | I-1474 |
| 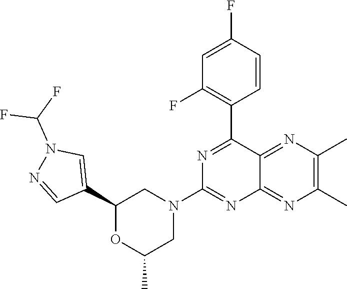 [SMILES: Cc1nc2nc(N3C[C@H](C)O[C@@H](c4cnn(C(F)F)c4)C3)nc(-c3ccc(F)cc3F)c2nc1C] | I-1475 |
| 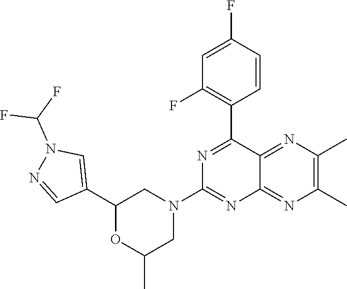 [SMILES: Cc1nc2nc(N3CC(C)OC(c4cnn(C(F)F)c4)C3)nc(-c3ccc(F)cc3F)c2nc1C] | I-1476 |
| 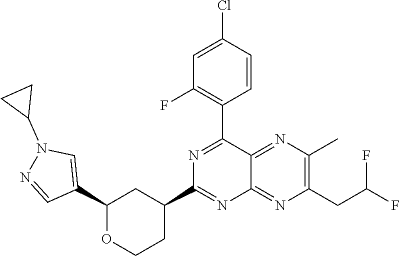 [SMILES: Cc1nc2c(-c3ccc(Cl)cc3F)nc([C@H]3CCO[C@@H](c4cnn(C5CC5)c4)C3)nc2nc1CC(F)F] | I-1477 |
| 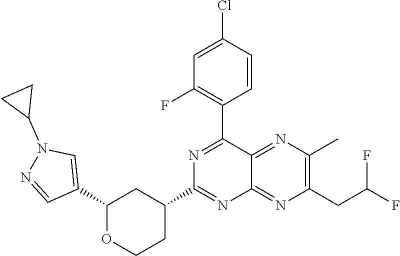 [SMILES: Cc1nc2c(-c3ccc(Cl)cc3F)nc([C@@H]3CCO[C@H](c4cnn(C5CC5)c4)C3)nc2nc1CC(F)F] | I-1478 |
| 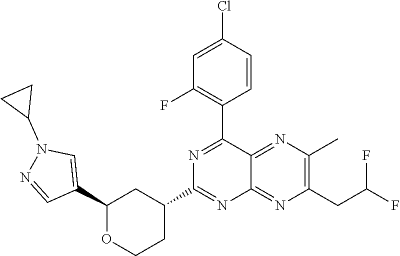 [SMILES: Cc1nc2c(-c3ccc(Cl)cc3F)nc([C@@H]3CCO[C@@H](c4cnn(C5CC5)c4)C3)nc2nc1CC(F)F] | I-1479 |
| 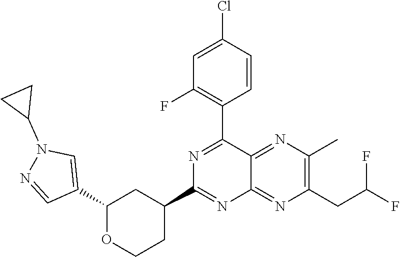 [SMILES: Cc1nc2c(-c3ccc(Cl)cc3F)nc([C@H]3CCO[C@H](c4cnn(C5CC5)c4)C3)nc2nc1CC(F)F] | I-1480 |
| 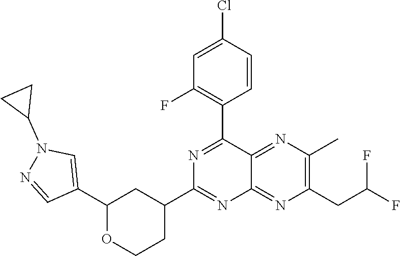 [SMILES: Cc1nc2c(-c3ccc(Cl)cc3F)nc(C3CCOC(c4cnn(C5CC5)c4)C3)nc2nc1CC(F)F] | I-1481 |
| 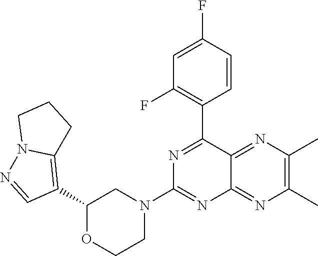 [SMILES: Cc1nc2nc(N3CCO[C@H](c4cnn5c4CCC5)C3)nc(-c3ccc(F)cc3F)c2nc1C] | I-1482 |
| 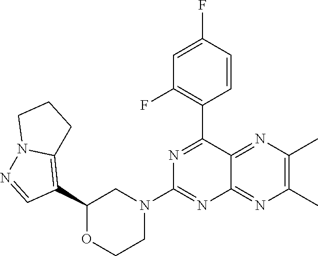 [SMILES: Cc1nc2nc(N3CCO[C@@H](c4cnn5c4CCC5)C3)nc(-c3ccc(F)cc3F)c2nc1C] | I-1483 |
| 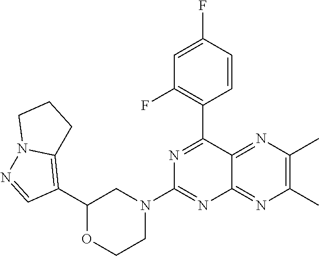 [SMILES: Cc1nc2nc(N3CCOC(c4cnn5c4CCC5)C3)nc(-c3ccc(F)cc3F)c2nc1C] | I-1484 |
| 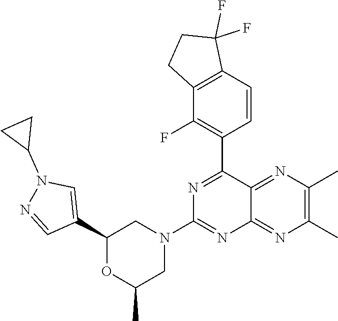 [SMILES: Cc1nc2nc(N3C[C@@H](C)O[C@@H](c4cnn(C5CC5)c4)C3)nc(-c3ccc4c(c3F)CCC4(F)F)c2nc1C] | I-1485 |
| 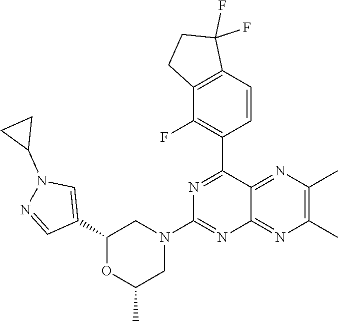 [SMILES: Cc1nc2nc(N3C[C@@H](c4cnn(C5CC5)c4)O[C@@H](C)C3)nc(-c3ccc4c(c3F)CCC4(F)F)c2nc1C] | I-1486 |
| 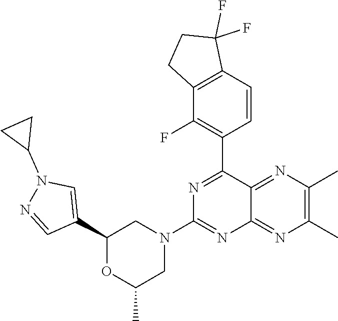 [SMILES: Cc1nc2nc(N3C[C@H](C)O[C@@H](c4cnn(C5CC5)c4)C3)nc(-c3ccc4c(c3F)CCC4(F)F)c2nc1C] | I-1487 |
| 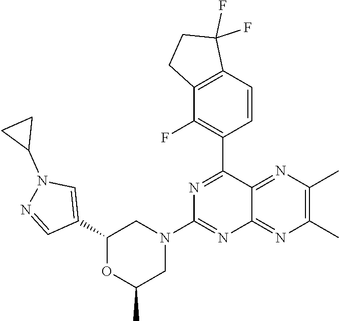 [SMILES: Cc1nc2nc(N3C[C@@H](C)O[C@H](c4cnn(C5CC5)c4)C3)nc(-c3ccc4c(c3F)CCC4(F)F)c2nc1C] | I-1488 |
| 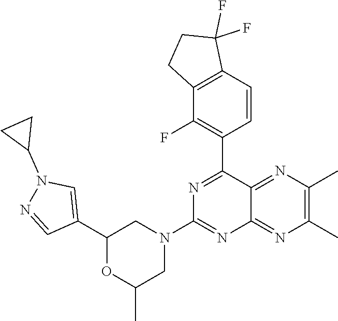 [SMILES: Cc1nc2nc(N3CC(C)OC(c4cnn(C5CC5)c4)C3)nc(-c3ccc4c(c3F)CCC4(F)F)c2nc1C] | I-1489 |
| 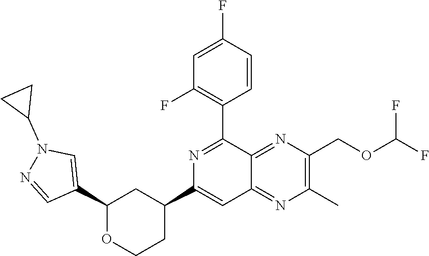 [SMILES: Cc1nc2cc([C@H]3CCO[C@@H](c4cnn(C5CC5)c4)C3)nc(-c3ccc(F)cc3F)c2nc1COC(F)F] | I-1490 |
| 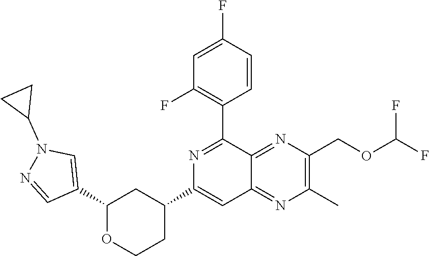 [SMILES: Cc1nc2cc([C@@H]3CCO[C@H](c4cnn(C5CC5)c4)C3)nc(-c3ccc(F)cc3F)c2nc1COC(F)F] | I-1491 |
| 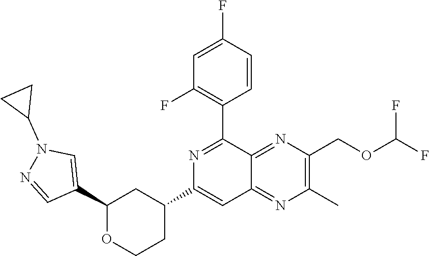 [SMILES: Cc1nc2cc([C@@H]3CCO[C@@H](c4cnn(C5CC5)c4)C3)nc(-c3ccc(F)cc3F)c2nc1COC(F)F] | I-1492 |
| 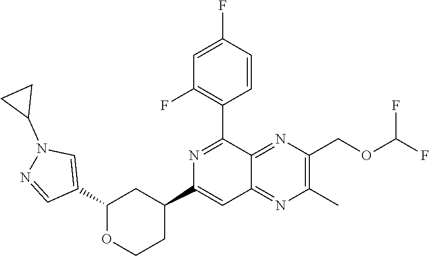 [SMILES: Cc1nc2cc([C@H]3CCO[C@H](c4cnn(C5CC5)c4)C3)nc(-c3ccc(F)cc3F)c2nc1COC(F)F] | I-1493 |
| 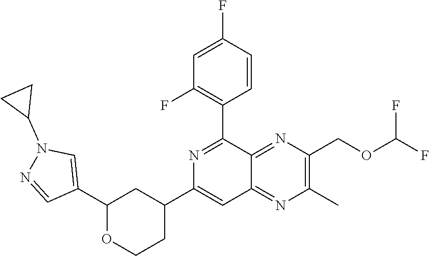 [SMILES: Cc1nc2cc(C3CCOC(c4cnn(C5CC5)c4)C3)nc(-c3ccc(F)cc3F)c2nc1COC(F)F] | I-1494 |
| 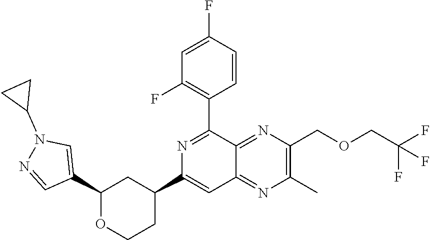 [SMILES: Cc1nc2cc([C@H]3CCO[C@@H](c4cnn(C5CC5)c4)C3)nc(-c3ccc(F)cc3F)c2nc1COCC(F)(F)F] | I-1495 |
| 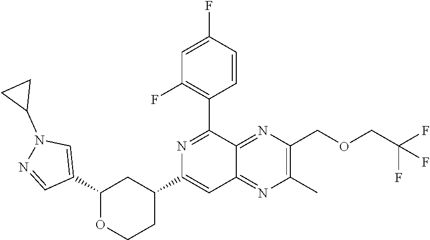 [SMILES: Cc1nc2cc([C@@H]3CCO[C@H](c4cnn(C5CC5)c4)C3)nc(-c3ccc(F)cc3F)c2nc1COCC(F)(F)F] | I-1496 |
| 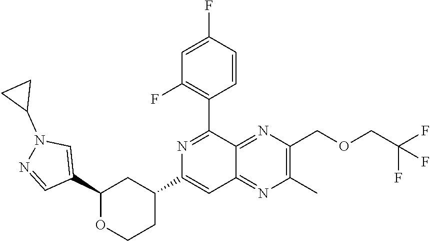 [SMILES: Cc1nc2cc([C@@H]3CCO[C@@H](c4cnn(C5CC5)c4)C3)nc(-c3ccc(F)cc3F)c2nc1COCC(F)(F)F] | I-1497 |
| 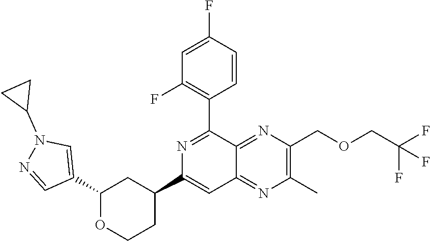 [SMILES: Cc1nc2cc([C@H]3CCO[C@H](c4cnn(C5CC5)c4)C3)nc(-c3ccc(F)cc3F)c2nc1COCC(F)(F)F] | I-1498 |
| 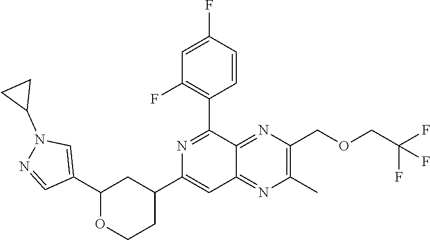 [SMILES: Cc1nc2cc(C3CCOC(c4cnn(C5CC5)c4)C3)nc(-c3ccc(F)cc3F)c2nc1COCC(F)(F)F] | I-1499 |
| 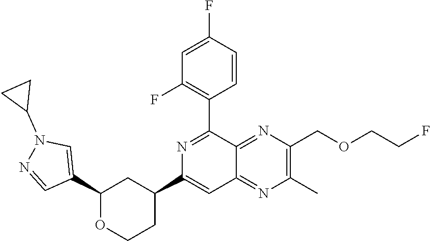 [SMILES: Cc1nc2cc([C@H]3CCO[C@@H](c4cnn(C5CC5)c4)C3)nc(-c3ccc(F)cc3F)c2nc1COCCF] | I-1500 |
| 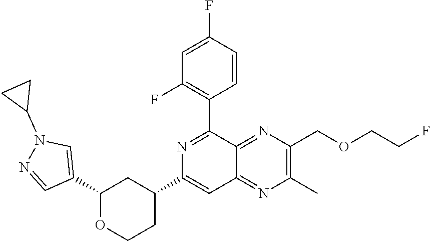 [SMILES: Cc1nc2cc([C@@H]3CCO[C@H](c4cnn(C5CC5)c4)C3)nc(-c3ccc(F)cc3F)c2nc1COCCF] | I-1501 |
| 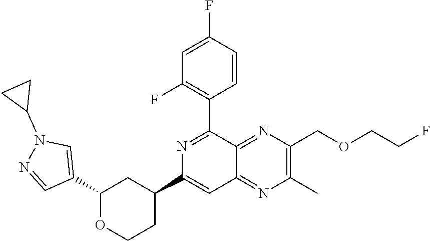 [SMILES: Cc1nc2cc([C@H]3CCO[C@H](c4cnn(C5CC5)c4)C3)nc(-c3ccc(F)cc3F)c2nc1COCCF] | I-1502 |
| 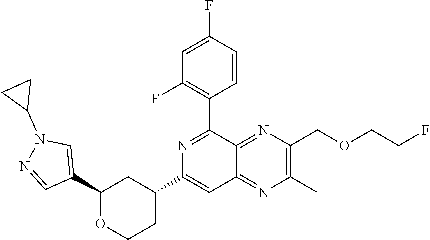 [SMILES: Cc1nc2cc([C@@H]3CCO[C@@H](c4cnn(C5CC5)c4)C3)nc(-c3ccc(F)cc3F)c2nc1COCCF] | I-1503 |
| 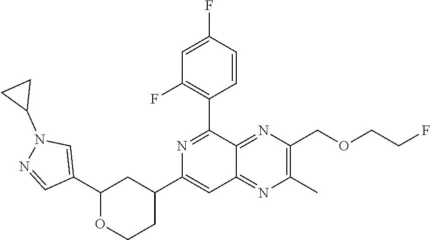 [SMILES: Cc1nc2cc(C3CCOC(c4cnn(C5CC5)c4)C3)nc(-c3ccc(F)cc3F)c2nc1COCCF] | I-1504 |
| 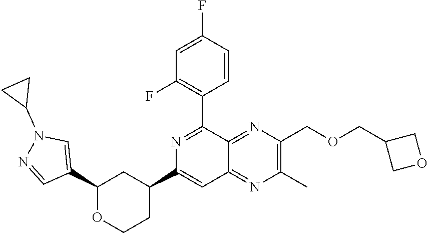 [SMILES: Cc1nc2cc([C@H]3CCO[C@@H](c4cnn(C5CC5)c4)C3)nc(-c3ccc(F)cc3F)c2nc1COCC1COC1] | I-1505 |
| 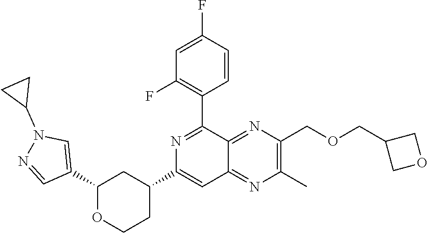 [SMILES: Cc1nc2cc([C@@H]3CCO[C@H](c4cnn(C5CC5)c4)C3)nc(-c3ccc(F)cc3F)c2nc1COCC1COC1] | I-1506 |
| 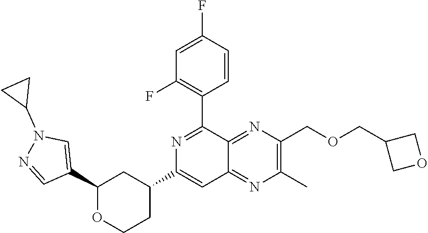 [SMILES: Cc1nc2cc([C@@H]3CCO[C@@H](c4cnn(C5CC5)c4)C3)nc(-c3ccc(F)cc3F)c2nc1COCC1COC1] | I-1507 |
| 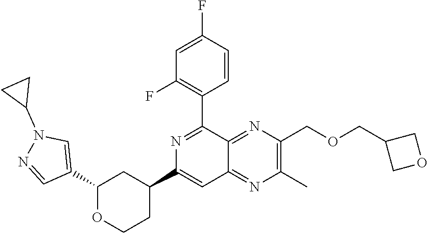 [SMILES: Cc1nc2cc([C@H]3CCO[C@H](c4cnn(C5CC5)c4)C3)nc(-c3ccc(F)cc3F)c2nc1COCC1COC1] | I-1508 |
| 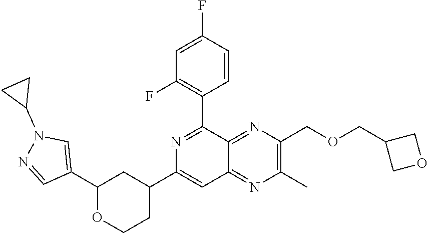 [SMILES: Cc1nc2cc(C3CCOC(c4cnn(C5CC5)c4)C3)nc(-c3ccc(F)cc3F)c2nc1COCC1COC1] | I-1509 |
| 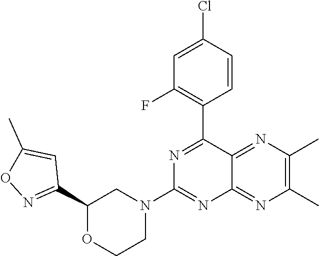 [SMILES: Cc1cc([C@H]2CN(c3nc(-c4ccc(Cl)cc4F)c4nc(C)c(C)nc4n3)CCO2)no1] | I-1510 |
| 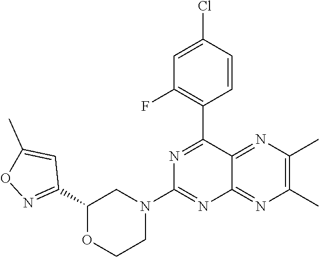 [SMILES: Cc1cc([C@@H]2CN(c3nc(-c4ccc(Cl)cc4F)c4nc(C)c(C)nc4n3)CCO2)no1] | I-1511 |
| 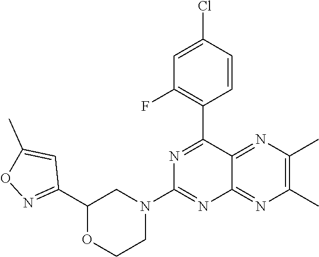 [SMILES: Cc1cc(C2CN(c3nc(-c4ccc(Cl)cc4F)c4nc(C)c(C)nc4n3)CCO2)no1] | I-1512 |
| 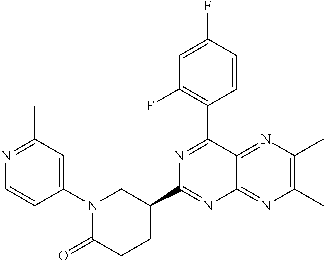 [SMILES: Cc1cc(N2C[C@@H](c3nc(-c4ccc(F)cc4F)c4nc(C)c(C)nc4n3)CCC2=O)ccn1] | I-1513 |
| 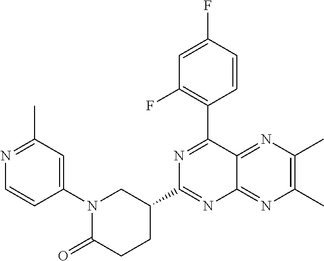 [SMILES: Cc1cc(N2C[C@H](c3nc(-c4ccc(F)cc4F)c4nc(C)c(C)nc4n3)CCC2=O)ccn1] | I-1514 |
| 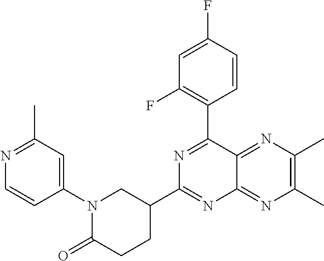 [SMILES: Cc1cc(N2CC(c3nc(-c4ccc(F)cc4F)c4nc(C)c(C)nc4n3)CCC2=O)ccn1] | I-1515 |
| 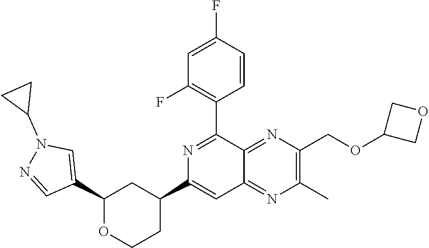 [SMILES: Cc1nc2cc([C@H]3CCO[C@@H](c4cnn(C5CC5)c4)C3)nc(-c3ccc(F)cc3F)c2nc1COC1COC1] | I-1516 |
| 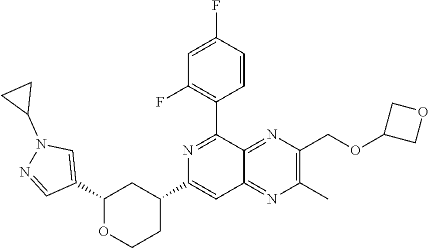 [SMILES: Cc1nc2cc([C@@H]3CCO[C@H](c4cnn(C5CC5)c4)C3)nc(-c3ccc(F)cc3F)c2nc1COC1COC1] | I-1517 |
| 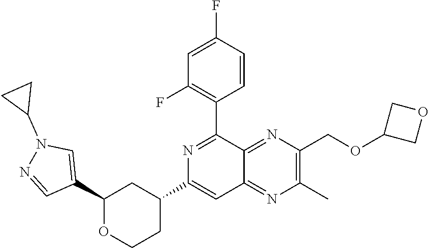 [SMILES: Cc1nc2cc([C@@H]3CCO[C@@H](c4cnn(C5CC5)c4)C3)nc(-c3ccc(F)cc3F)c2nc1COC1COC1] | I-1518 |
| 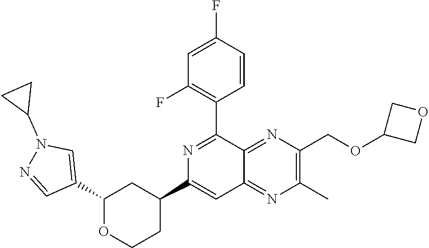 [SMILES: Cc1nc2cc([C@H]3CCO[C@H](c4cnn(C5CC5)c4)C3)nc(-c3ccc(F)cc3F)c2nc1COC1COC1] | I-1519 |
| 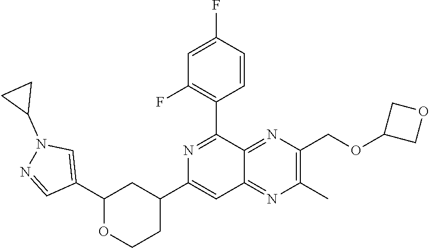 [SMILES: Cc1nc2cc(C3CCOC(c4cnn(C5CC5)c4)C3)nc(-c3ccc(F)cc3F)c2nc1COC1COC1] | I-1520 |
| 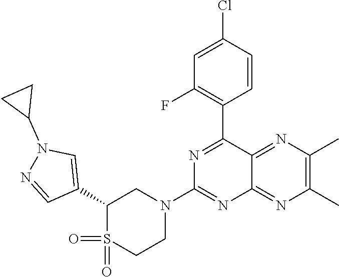 [SMILES: Cc1nc2nc(N3CCS(=O)(=O)[C@H](c4cnn(C5CC5)c4)C3)nc(-c3ccc(Cl)cc3F)c2nc1C] | I-1521 |
| 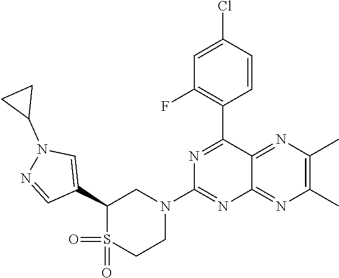 [SMILES: Cc1nc2nc(N3CCS(=O)(=O)[C@@H](c4cnn(C5CC5)c4)C3)nc(-c3ccc(Cl)cc3F)c2nc1C] | I-1522 |
| 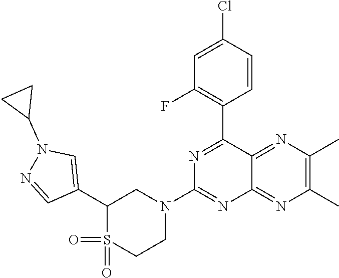 [SMILES: Cc1nc2nc(N3CCS(=O)(=O)C(c4cnn(C5CC5)c4)C3)nc(-c3ccc(Cl)cc3F)c2nc1C] | I-1523 |
| 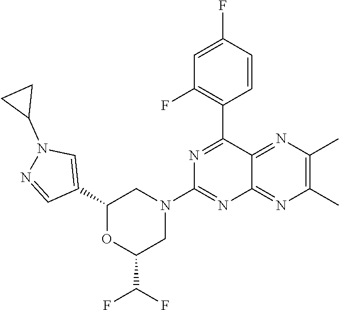 [SMILES: Cc1nc2nc(N3C[C@@H](c4cnn(C5CC5)c4)O[C@@H](C(F)F)C3)nc(-c3ccc(F)cc3F)c2nc1C] | I-1524 |
| 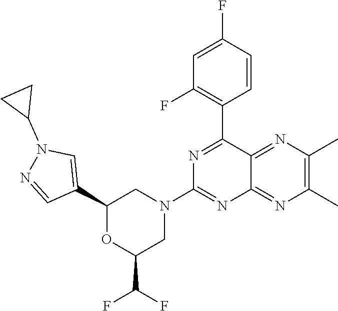 [SMILES: Cc1nc2nc(N3C[C@@H](C(F)F)O[C@@H](c4cnn(C5CC5)c4)C3)nc(-c3ccc(F)cc3F)c2nc1C] | I-1525 |
| 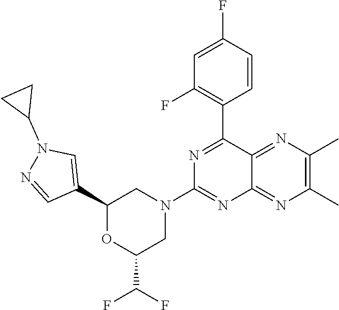 [SMILES: Cc1nc2nc(N3C[C@H](c4cnn(C5CC5)c4)O[C@@H](C(F)F)C3)nc(-c3ccc(F)cc3F)c2nc1C] | I-1526 |
| 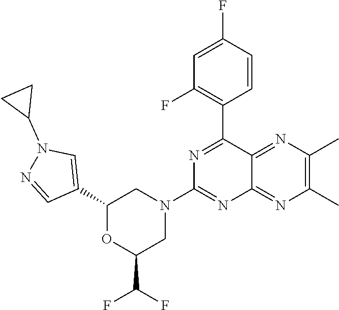 [SMILES: Cc1nc2nc(N3C[C@@H](c4cnn(C5CC5)c4)O[C@H](C(F)F)C3)nc(-c3ccc(F)cc3F)c2nc1C] | I-1527 |
| 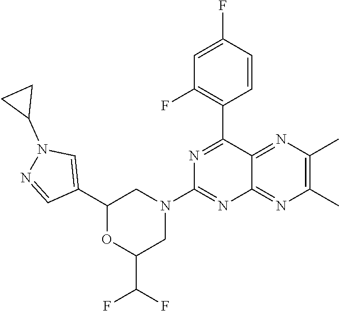 [SMILES: Cc1nc2nc(N3CC(c4cnn(C5CC5)c4)OC(C(F)F)C3)nc(-c3ccc(F)cc3F)c2nc1C] | I-1528 |
| 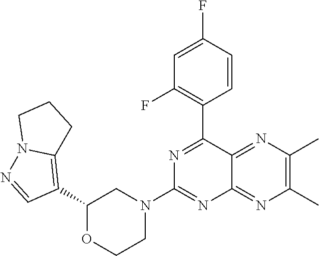 [SMILES: Cc1nc2nc(N3CCO[C@H](c4cnn5c4CCC5)C3)nc(-c3ccc(F)cc3F)c2nc1C] | I-1529 |
| 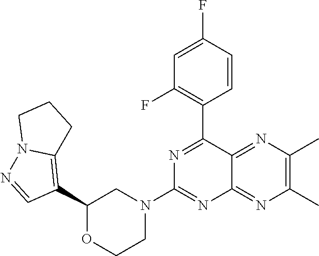 [SMILES: Cc1nc2nc(N3CCO[C@@H](c4cnn5c4CCC5)C3)nc(-c3ccc(F)cc3F)c2nc1C] | I-1530 |
| 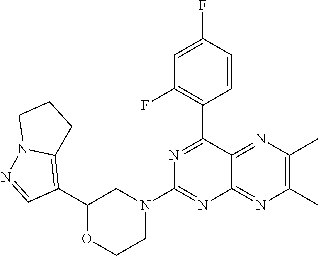 [SMILES: Cc1nc2nc(N3CCOC(c4cnn5c4CCC5)C3)nc(-c3ccc(F)cc3F)c2nc1C] | I-1531 |
| 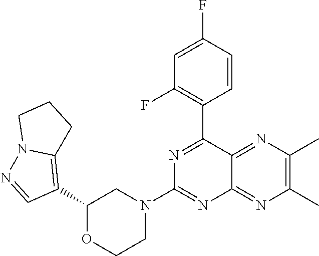 [SMILES: Cc1nc2nc(N3CCO[C@H](c4cnn5c4CCC5)C3)nc(-c3ccc(F)cc3F)c2nc1C] | I-1532 |
| 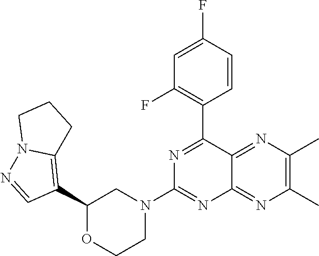 [SMILES: Cc1nc2nc(N3CCO[C@@H](c4cnn5c4CCC5)C3)nc(-c3ccc(F)cc3F)c2nc1C] | I-1533 |
| 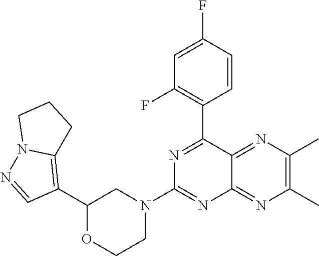 [SMILES: Cc1nc2nc(N3CCOC(c4cnn5c4CCC5)C3)nc(-c3ccc(F)cc3F)c2nc1C] | I-1534 |
| 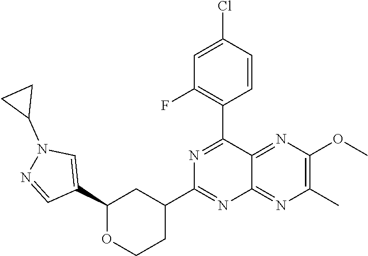 [SMILES: COc1nc2c(-c3ccc(Cl)cc3F)nc(C3CCO[C@@H](c4cnn(C5CC5)c4)C3)nc2nc1C] | I-1535 |
| 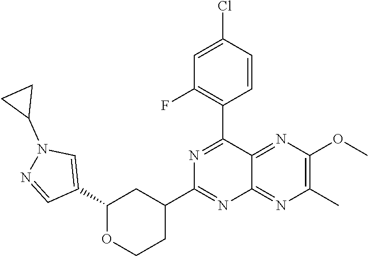 [SMILES: COc1nc2c(-c3ccc(Cl)cc3F)nc(C3CCO[C@H](c4cnn(C5CC5)c4)C3)nc2nc1C] | I-1536 |
| 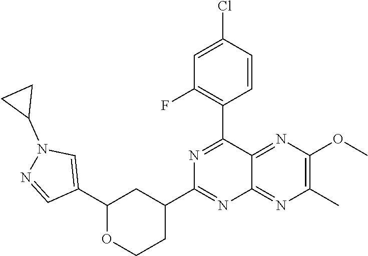 [SMILES: COc1nc2c(-c3ccc(Cl)cc3F)nc(C3CCOC(c4cnn(C5CC5)c4)C3)nc2nc1C] | I-1537 |
| 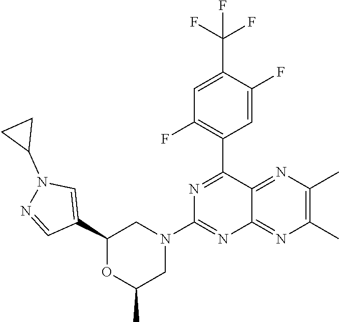 [SMILES: Cc1nc2nc(N3C[C@@H](C)O[C@@H](c4cnn(C5CC5)c4)C3)nc(-c3cc(F)c(C(F)(F)F)cc3F)c2nc1C] | I-1538 |
| 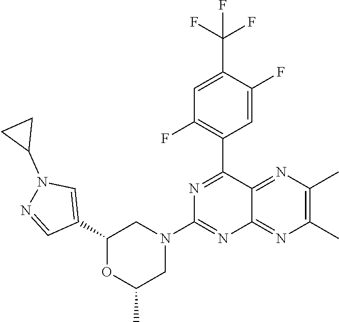 [SMILES: Cc1nc2nc(N3C[C@@H](c4cnn(C5CC5)c4)O[C@@H](C)C3)nc(-c3cc(F)c(C(F)(F)F)cc3F)c2nc1C] | I-1539 |
| 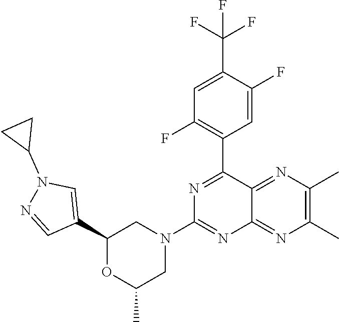 [SMILES: Cc1nc2nc(N3C[C@H](C)O[C@@H](c4cnn(C5CC5)c4)C3)nc(-c3cc(F)c(C(F)(F)F)cc3F)c2nc1C] | I-1540 |
| 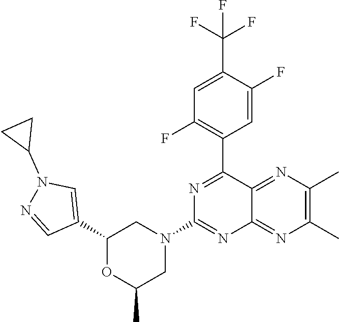 | I-1541 |
| 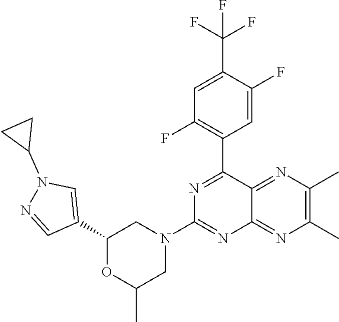 [SMILES: Cc1nc2nc(N3CC(C)O[C@H](c4cnn(C5CC5)c4)C3)nc(-c3cc(F)c(C(F)(F)F)cc3F)c2nc1C] | I-1542 |
| 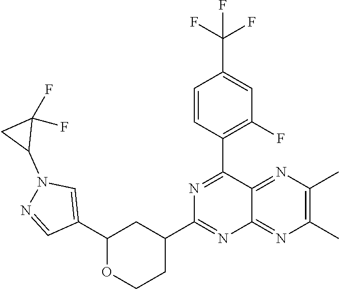 [SMILES: Cc1nc2nc(C3CCOC(c4cnn(C5CC5(F)F)c4)C3)nc(-c3ccc(C(F)(F)F)cc3F)c2nc1C] | I-1543 |
| 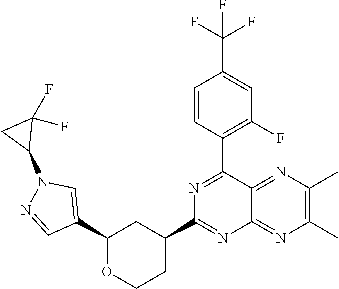 [SMILES: Cc1nc2nc([C@H]3CCO[C@@H](c4cnn([C@H]5CC5(F)F)c4)C3)nc(-c3ccc(C(F)(F)F)cc3F)c2nc1C] | I-1544 |
| 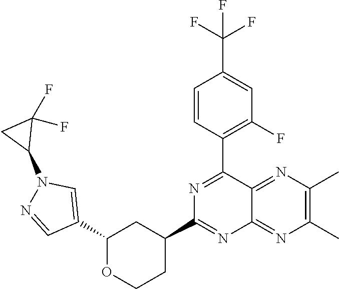 [SMILES: Cc1nc2nc([C@H]3CCO[C@H](c4cnn([C@H]5CC5(F)F)c4)C3)nc(-c3ccc(C(F)(F)F)cc3F)c2nc1C] | I-1545 |
| 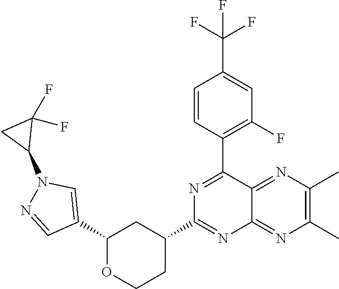 [SMILES: Cc1nc2nc([C@@H]3CCO[C@H](c4cnn([C@H]5CC5(F)F)c4)C3)nc(-c3ccc(C(F)(F)F)cc3F)c2nc1C] | I-1546 |
| 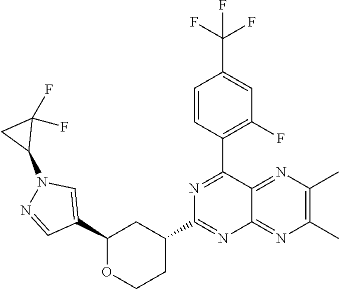 [SMILES: Cc1nc2nc([C@@H]3CCO[C@@H](c4cnn([C@H]5CC5(F)F)c4)C3)nc(-c3ccc(C(F)(F)F)cc3F)c2nc1C] | I-1547 |
| 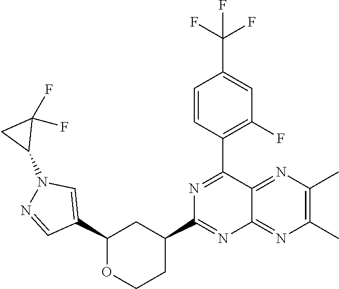 [SMILES: Cc1nc2nc([C@H]3CCO[C@@H](c4cnn([C@@H]5CC5(F)F)c4)C3)nc(-c3ccc(C(F)(F)F)cc3F)c2nc1C] | I-1548 |
| 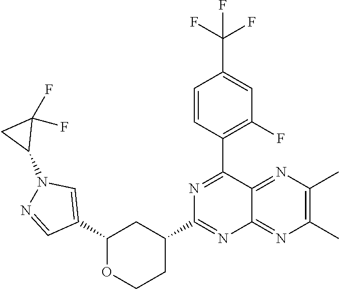 [SMILES: Cc1nc2nc([C@@H]3CCO[C@H](c4cnn([C@@H]5CC5(F)F)c4)C3)nc(-c3ccc(C(F)(F)F)cc3F)c2nc1C] | I-1549 |
| 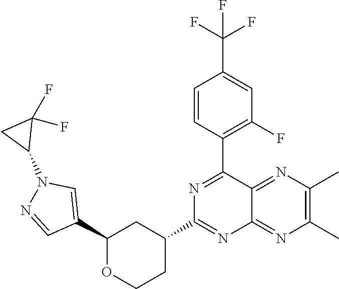 [SMILES: Cc1nc2nc([C@@H]3CCO[C@@H](c4cnn([C@@H]5CC5(F)F)c4)C3)nc(-c3ccc(C(F)(F)F)cc3F)c2nc1C] | I-1550 |
| 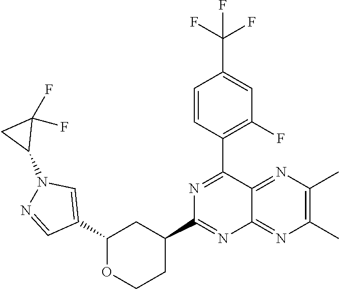 [SMILES: Cc1nc2nc([C@H]3CCO[C@H](c4cnn([C@@H]5CC5(F)F)c4)C3)nc(-c3ccc(C(F)(F)F)cc3F)c2nc1C] | I-1551 |
| 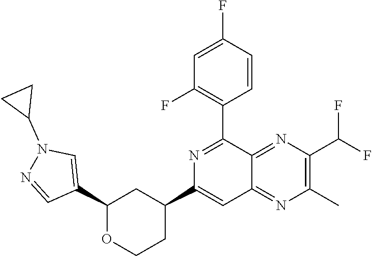 [SMILES: Cc1nc2cc([C@H]3CCO[C@@H](c4cnn(C5CC5)c4)C3)nc(-c3ccc(F)cc3F)c2nc1C(F)F] | I-1552 |
| 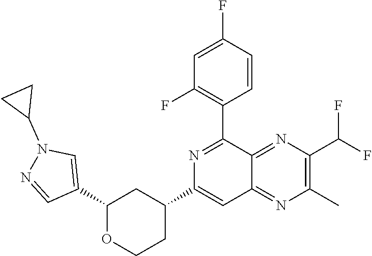 [SMILES: Cc1nc2cc([C@@H]3CCO[C@H](c4cnn(C5CC5)c4)C3)nc(-c3ccc(F)cc3F)c2nc1C(F)F] | I-1553 |
| 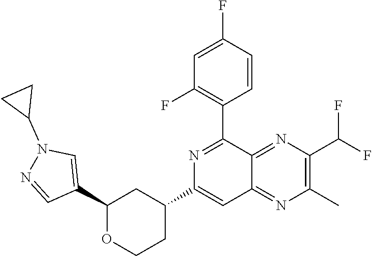 [SMILES: Cc1nc2cc([C@@H]3CCO[C@@H](c4cnn(C5CC5)c4)C3)nc(-c3ccc(F)cc3F)c2nc1C(F)F] | I-1554 |
| 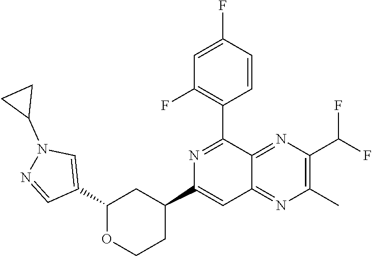 [SMILES: Cc1nc2cc([C@H]3CCO[C@H](c4cnn(C5CC5)c4)C3)nc(-c3ccc(F)cc3F)c2nc1C(F)F] | I-1555 |
| 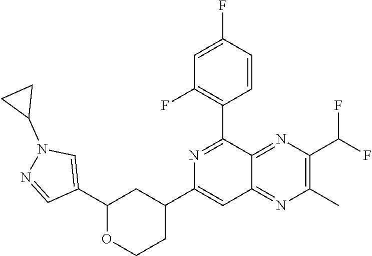 [SMILES: Cc1nc2cc(C3CCOC(c4cnn(C5CC5)c4)C3)nc(-c3ccc(F)cc3F)c2nc1C(F)F] | I-1556 |
| 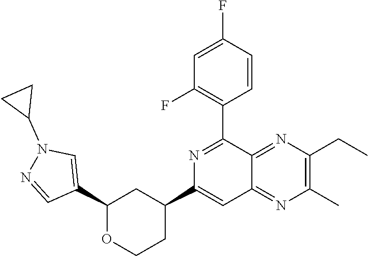 [SMILES: CCc1nc2c(-c3ccc(F)cc3F)nc([C@H]3CCO[C@@H](c4cnn(C5CC5)c4)C3)cc2nc1C] | I-1557 |
| 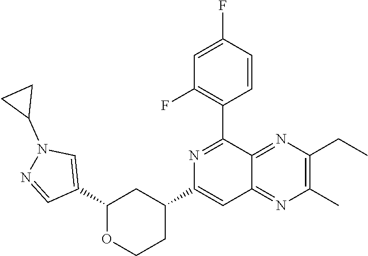 [SMILES: CCc1nc2c(-c3ccc(F)cc3F)nc([C@@H]3CCO[C@H](c4cnn(C5CC5)c4)C3)cc2nc1C] | I-1558 |
| 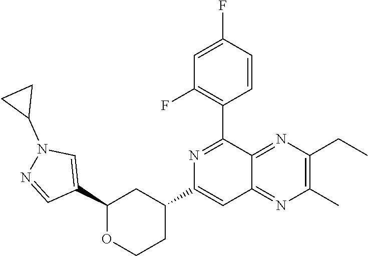 [SMILES: CCc1nc2c(-c3ccc(F)cc3F)nc([C@@H]3CCO[C@@H](c4cnn(C5CC5)c4)C3)cc2nc1C] | I-1559 |
| 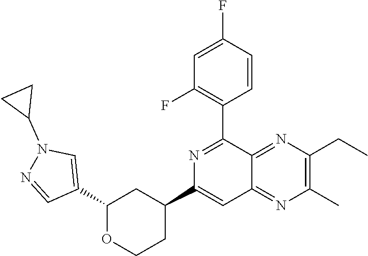 [SMILES: CCc1nc2c(-c3ccc(F)cc3F)nc([C@H]3CCO[C@H](c4cnn(C5CC5)c4)C3)cc2nc1C] | I-1560 |
| 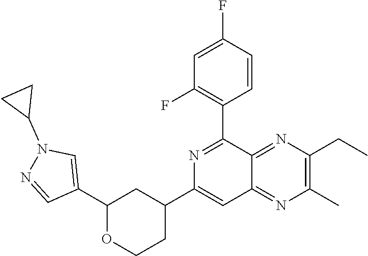 [SMILES: CCc1nc2c(-c3ccc(F)cc3F)nc(C3CCOC(c4cnn(C5CC5)c4)C3)cc2nc1C] | I-1561 |
| 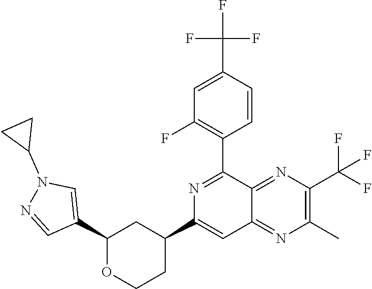 [SMILES: Cc1nc2cc([C@H]3CCO[C@@H](c4cnn(C5CC5)c4)C3)nc(-c3ccc(C(F)(F)F)cc3F)c2nc1C(F)(F)F] | I-1562 |
| 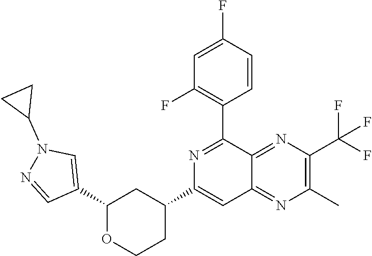 [SMILES: Cc1nc2cc([C@@H]3CCO[C@H](c4cnn(C5CC5)c4)C3)nc(-c3ccc(F)cc3F)c2nc1C(F)(F)F] | I-1563 |
| 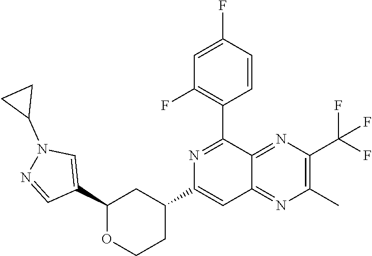 [SMILES: Cc1nc2cc([C@@H]3CCO[C@@H](c4cnn(C5CC5)c4)C3)nc(-c3ccc(F)cc3F)c2nc1C(F)(F)F] | I-1564 |
| 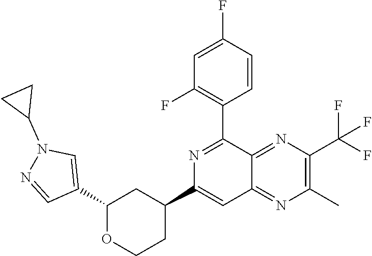 [SMILES: Cc1nc2cc([C@H]3CCO[C@H](c4cnn(C5CC5)c4)C3)nc(-c3ccc(F)cc3F)c2nc1C(F)(F)F] | I-1565 |
| 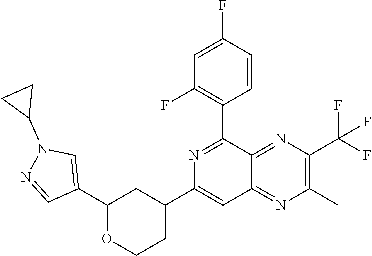 [SMILES: Cc1nc2cc(C3CCOC(c4cnn(C5CC5)c4)C3)nc(-c3ccc(F)cc3F)c2nc1C(F)(F)F] | I-1566 |
| 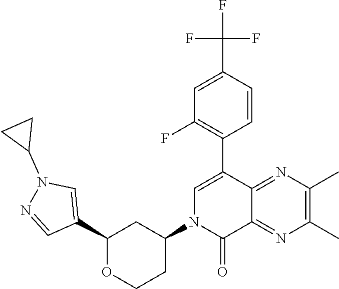 [SMILES: Cc1nc2c(-c3ccc(C(F)(F)F)cc3F)cn([C@H]3CCO[C@@H](c4cnn(C5CC5)c4)C3)c(=O)c2nc1C] | I-1567 |
| 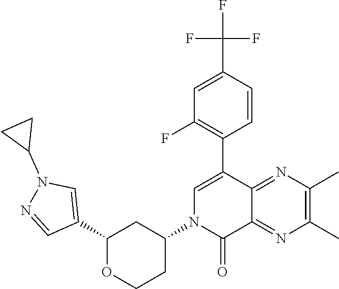 [SMILES: Cc1nc2c(-c3ccc(C(F)(F)F)cc3F)cn([C@@H]3CCO[C@H](c4cnn(C5CC5)c4)C3)c(=O)c2nc1C] | I-1568 |
| 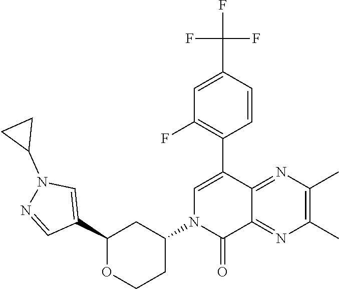 [SMILES: Cc1nc2c(-c3ccc(C(F)(F)F)cc3F)cn([C@@H]3CCO[C@@H](c4cnn(C5CC5)c4)C3)c(=O)c2nc1C] | I-1569 |
| 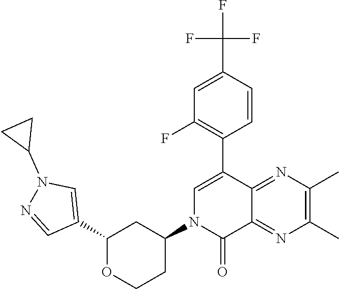 [SMILES: Cc1nc2c(-c3ccc(C(F)(F)F)cc3F)cn([C@H]3CCO[C@H](c4cnn(C5CC5)c4)C3)c(=O)c2nc1C] | I-1570 |
| 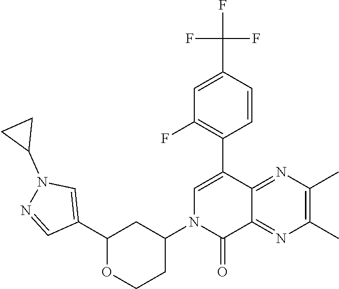 [SMILES: Cc1nc2c(-c3ccc(C(F)(F)F)cc3F)cn(C3CCOC(c4cnn(C5CC5)c4)C3)c(=O)c2nc1C] | I-1571 |
| 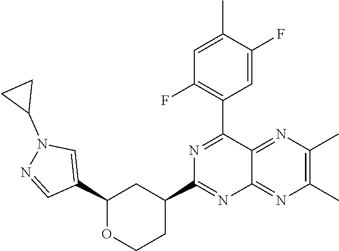 [SMILES: Cc1cc(F)c(-c2nc([C@H]3CCO[C@@H](c4cnn(C5CC5)c4)C3)nc3nc(C)c(C)nc23)cc1F] | I-1572 |
| 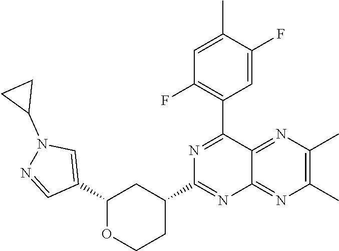 [SMILES: Cc1cc(F)c(-c2nc([C@@H]3CCO[C@H](c4cnn(C5CC5)c4)C3)nc3nc(C)c(C)nc23)cc1F] | I-1573 |
| 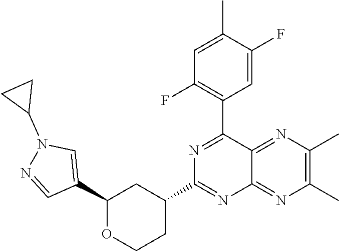 [SMILES: Cc1cc(F)c(-c2nc([C@@H]3CCO[C@@H](c4cnn(C5CC5)c4)C3)nc3nc(C)c(C)nc23)cc1F] | I-1574 |
| 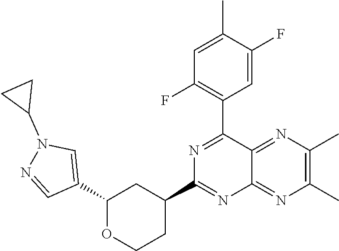 [SMILES: Cc1cc(F)c(-c2nc([C@H]3CCO[C@H](c4cnn(C5CC5)c4)C3)nc3nc(C)c(C)nc23)cc1F] | I-1575 |
| 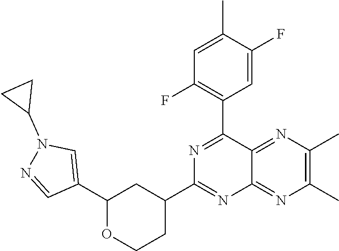 [SMILES: Cc1cc(F)c(-c2nc(C3CCOC(c4cnn(C5CC5)c4)C3)nc3nc(C)c(C)nc23)cc1F] | I-1576 |
| 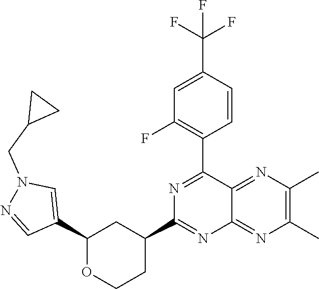 [SMILES: Cc1nc2nc([C@H]3CCO[C@@H](c4cnn(CC5CC5)c4)C3)nc(-c3ccc(C(F)(F)F)cc3F)c2nc1C] | I-1577 |
| 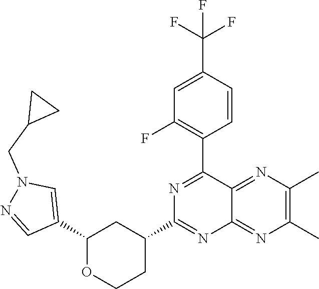 [SMILES: Cc1nc2nc([C@@H]3CCO[C@H](c4cnn(CC5CC5)c4)C3)nc(-c3ccc(C(F)(F)F)cc3F)c2nc1C] | I-1578 |
| 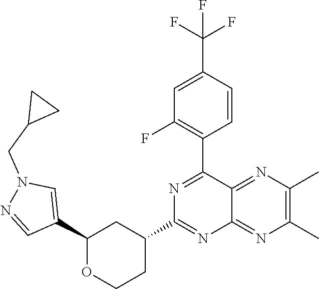 [SMILES: Cc1nc2nc([C@@H]3CCO[C@@H](c4cnn(CC5CC5)c4)C3)nc(-c3ccc(C(F)(F)F)cc3F)c2nc1C] | I-1579 |
| 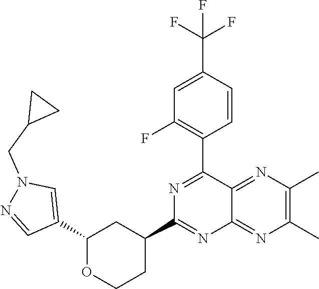 [SMILES: Cc1nc2nc([C@H]3CCO[C@H](c4cnn(CC5CC5)c4)C3)nc(-c3ccc(C(F)(F)F)cc3F)c2nc1C] | I-1580 |
| 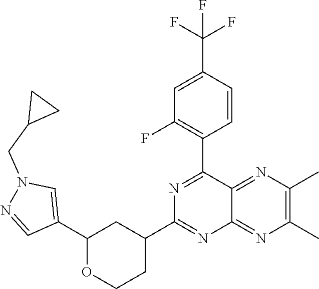 [SMILES: Cc1nc2nc(C3CCOC(c4cnn(CC5CC5)c4)C3)nc(-c3ccc(C(F)(F)F)cc3F)c2nc1C] | I-1581 |
| 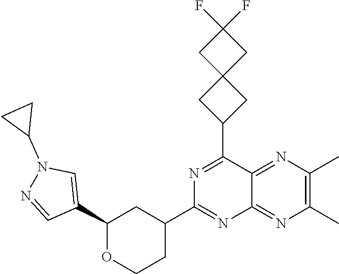 [SMILES: Cc1nc2nc(C3CCO[C@@H](c4cnn(C5CC5)c4)C3)nc(C3CC4(C3)CC(F)(F)C4)c2nc1C] | I-1582 |
| 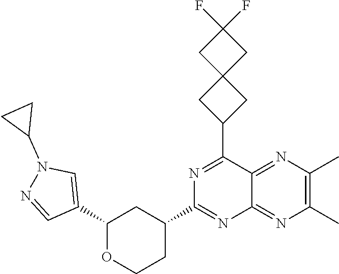 [SMILES: Cc1nc2nc([C@@H]3CCO[C@H](c4cnn(C5CC5)c4)C3)nc(C3CC4(C3)CC(F)(F)C4)c2nc1C] | I-1583 |
| 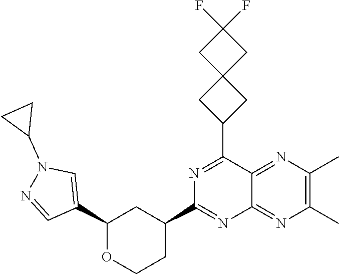 [SMILES: Cc1nc2nc([C@H]3CCO[C@@H](c4cnn(C5CC5)c4)C3)nc(C3CC4(C3)CC(F)(F)C4)c2nc1C] | I-1584 |
| 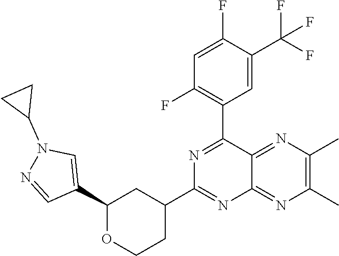 [SMILES: Cc1nc2nc(C3CCO[C@@H](c4cnn(C5CC5)c4)C3)nc(-c3cc(C(F)(F)F)c(F)cc3F)c2nc1C] | I-1585 |
| 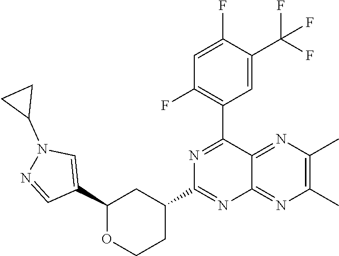 [SMILES: Cc1nc2nc([C@@H]3CCO[C@@H](c4cnn(C5CC5)c4)C3)nc(-c3cc(C(F)(F)F)c(F)cc3F)c2nc1C] | I-1586 |
| 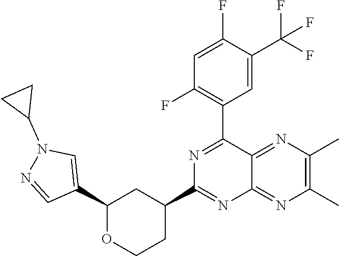 [SMILES: Cc1nc2nc([C@H]3CCO[C@@H](c4cnn(C5CC5)c4)C3)nc(-c3cc(C(F)(F)F)c(F)cc3F)c2nc1C] | I-1587 |
| 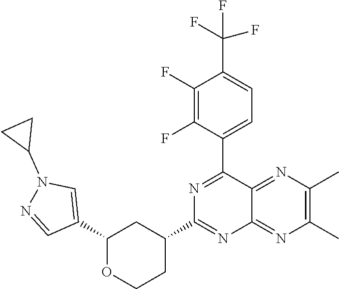 [SMILES: Cc1nc2nc([C@@H]3CCO[C@H](c4cnn(C5CC5)c4)C3)nc(-c3ccc(C(F)(F)F)c(F)c3F)c2nc1C] | I-1588 |
| 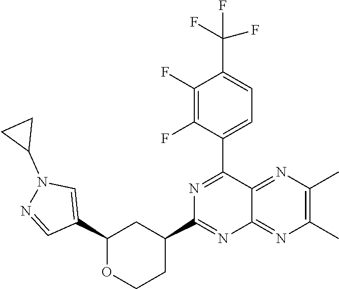 [SMILES: Cc1nc2nc([C@H]3CCO[C@@H](c4cnn(C5CC5)c4)C3)nc(-c3ccc(C(F)(F)F)c(F)c3F)c2nc1C] | I-1589 |
| 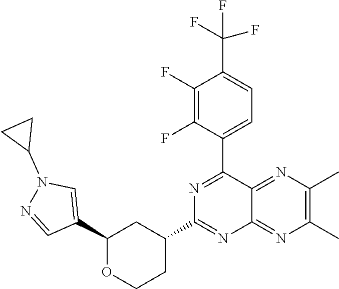 [SMILES: Cc1nc2nc([C@@H]3CCO[C@@H](c4cnn(C5CC5)c4)C3)nc(-c3ccc(C(F)(F)F)c(F)c3F)c2nc1C] | I-1590 |
| 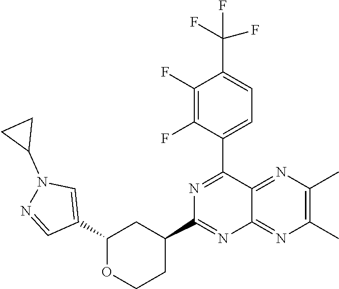 [SMILES: Cc1nc2nc([C@H]3CCO[C@H](c4cnn(C5CC5)c4)C3)nc(-c3ccc(C(F)(F)F)c(F)c3F)c2nc1C] | I-1591 |
| 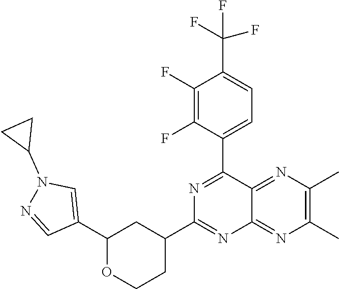 [SMILES: Cc1nc2nc(C3CCOC(c4cnn(C5CC5)c4)C3)nc(-c3ccc(C(F)(F)F)c(F)c3F)c2nc1C] | I-1592 |
| 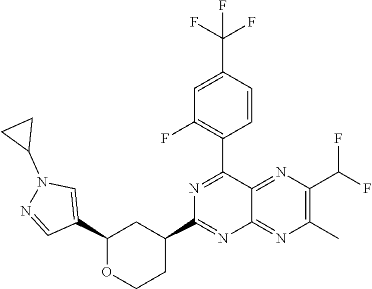 [SMILES: Cc1nc2nc([C@H]3CCO[C@@H](c4cnn(C5CC5)c4)C3)nc(-c3ccc(C(F)(F)F)cc3F)c2nc1C(F)F] | I-1593 |
| 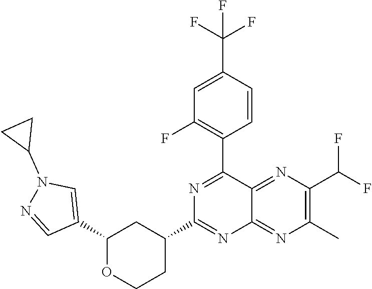 [SMILES: Cc1nc2nc([C@@H]3CCO[C@H](c4cnn(C5CC5)c4)C3)nc(-c3ccc(C(F)(F)F)cc3F)c2nc1C(F)F] | I-1594 |
| 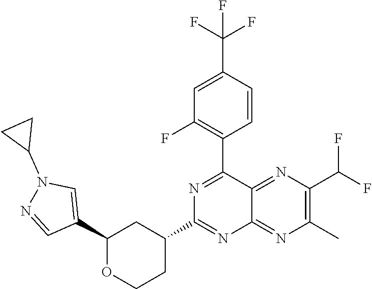 [SMILES: Cc1nc2nc([C@@H]3CCO[C@@H](c4cnn(C5CC5)c4)C3)nc(-c3ccc(C(F)(F)F)cc3F)c2nc1C(F)F] | I-1595 |
| 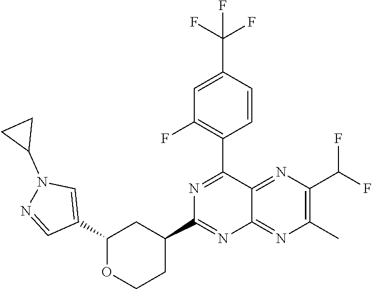 [SMILES: Cc1nc2nc([C@H]3CCO[C@H](c4cnn(C5CC5)c4)C3)nc(-c3ccc(C(F)(F)F)cc3F)c2nc1C(F)F] | I-1596 |
| 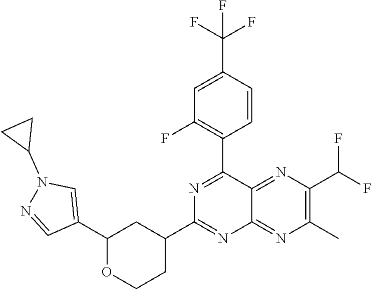 [SMILES: Cc1nc2nc(C3CCOC(c4cnn(C5CC5)c4)C3)nc(-c3ccc(C(F)(F)F)cc3F)c2nc1C(F)F] | I-1597 |
| 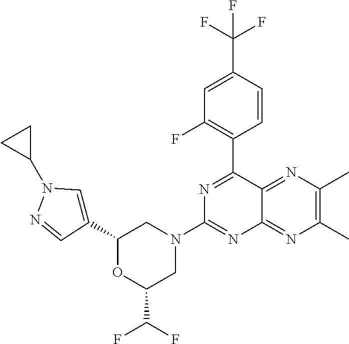 [SMILES: Cc1nc2nc(N3C[C@@H](c4cnn(C5CC5)c4)O[C@@H](C(F)F)C3)nc(-c3ccc(C(F)(F)F)cc3F)c2nc1C] | I-1598 |
| 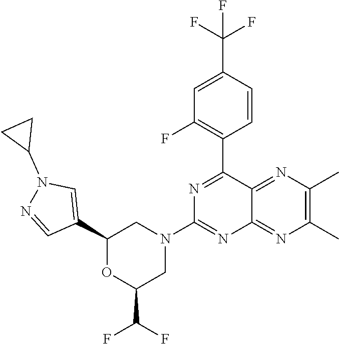 [SMILES: Cc1nc2nc(N3C[C@@H](C(F)F)O[C@@H](c4cnn(C5CC5)c4)C3)nc(-c3ccc(C(F)(F)F)cc3F)c2nc1C] | I-1599 |
| 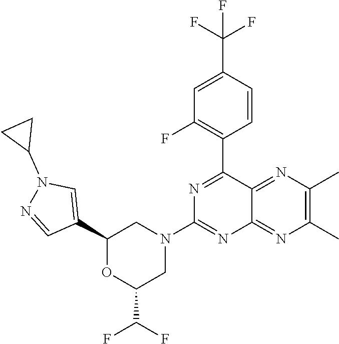 [SMILES: Cc1nc2nc(N3C[C@H](c4cnn(C5CC5)c4)O[C@@H](C(F)F)C3)nc(-c3ccc(C(F)(F)F)cc3F)c2nc1C] | I-1600 |
| 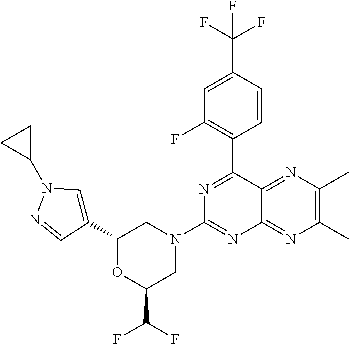 [SMILES: Cc1nc2nc(N3C[C@@H](c4cnn(C5CC5)c4)O[C@H](C(F)F)C3)nc(-c3ccc(C(F)(F)F)cc3F)c2nc1C] | I-1601 |
| 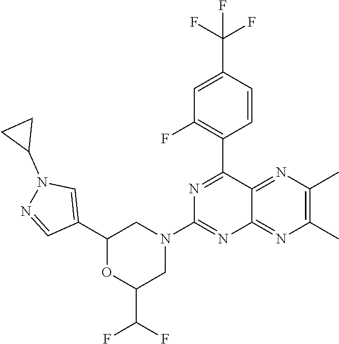 [SMILES: Cc1nc2nc(N3CC(c4cnn(C5CC5)c4)OC(C(F)F)C3)nc(-c3ccc(C(F)(F)F)cc3F)c2nc1C] | I-1602 |
| 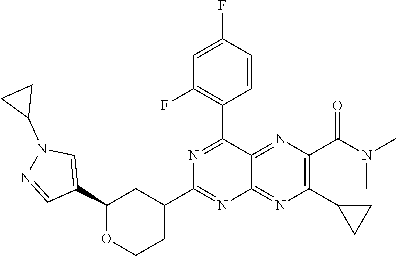 [SMILES: CN(C)C(=O)c1nc2c(-c3ccc(F)cc3F)nc(C3CCO[C@@H](c4cnn(C5CC5)c4)C3)nc2nc1C1CC1] | I-1603 |
| 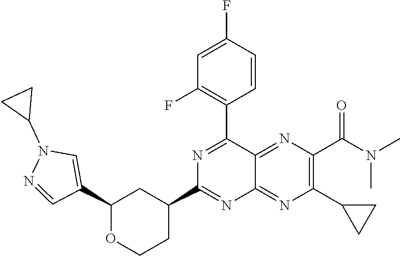 [SMILES: CN(C)C(=O)c1nc2c(-c3ccc(F)cc3F)nc([C@H]3CCO[C@@H](c4cnn(C5CC5)c4)C3)nc2nc1C1CC1] | I-1604 |
| 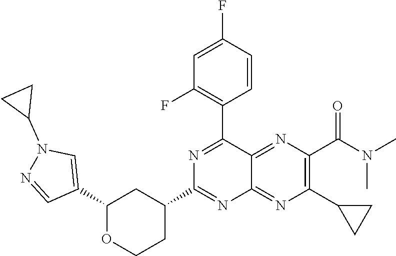 [SMILES: CN(C)C(=O)c1nc2c(-c3ccc(F)cc3F)nc([C@@H]3CCO[C@H](c4cnn(C5CC5)c4)C3)nc2nc1C1CC1] | I-1605 |
| 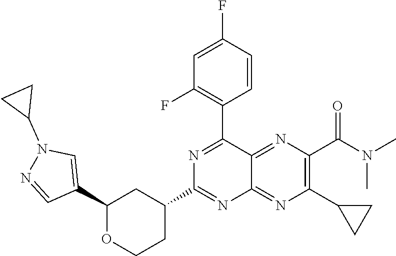 [SMILES: CN(C)C(=O)c1nc2c(-c3ccc(F)cc3F)nc([C@@H]3CCO[C@@H](c4cnn(C5CC5)c4)C3)nc2nc1C1CC1] | I-1606 |
| 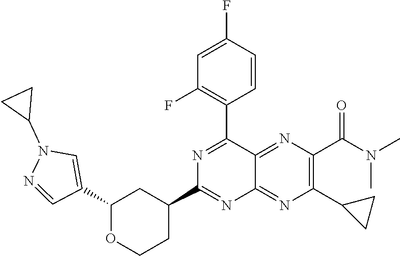 [SMILES: CN(C)C(=O)c1nc2c(-c3ccc(F)cc3F)nc([C@H]3CCO[C@H](c4cnn(C5CC5)c4)C3)nc2nc1C1CC1] | I-1607 |
| 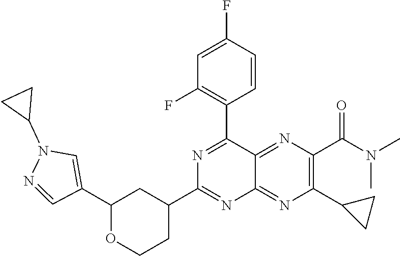 [SMILES: CN(C)C(=O)c1nc2c(-c3ccc(F)cc3F)nc(C3CCOC(c4cnn(C5CC5)c4)C3)nc2nc1C1CC1] | I-1608 |
| 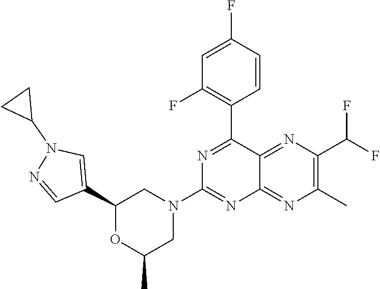 [SMILES: Cc1nc2nc(N3C[C@@H](C)O[C@@H](c4cnn(C5CC5)c4)C3)nc(-c3ccc(F)cc3F)c2nc1C(F)F] | I-1609 |
| 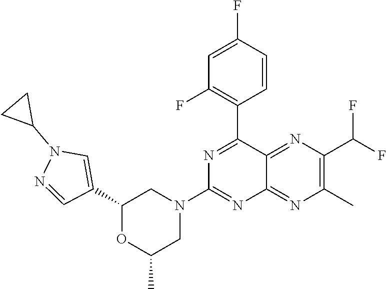 [SMILES: Cc1nc2nc(N3C[C@@H](c4cnn(C5CC5)c4)O[C@@H](C)C3)nc(-c3ccc(F)cc3F)c2nc1C(F)F] | I-1610 |
| 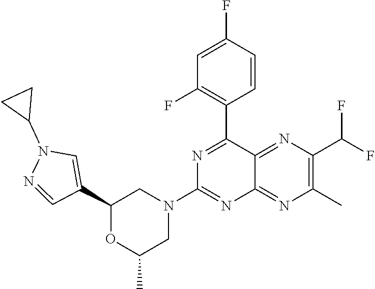 [SMILES: Cc1nc2nc(N3C[C@H](C)O[C@@H](c4cnn(C5CC5)c4)C3)nc(-c3ccc(F)cc3F)c2nc1C(F)F] | I-1611 |
| 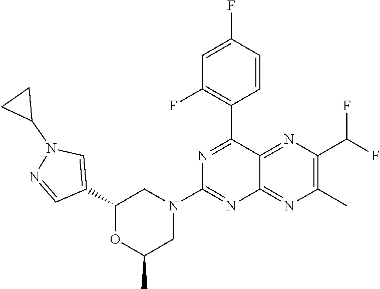 [SMILES: Cc1nc2nc(N3C[C@@H](C)O[C@H](c4cnn(C5CC5)c4)C3)nc(-c3ccc(F)cc3F)c2nc1C(F)F] | I-1612 |
| 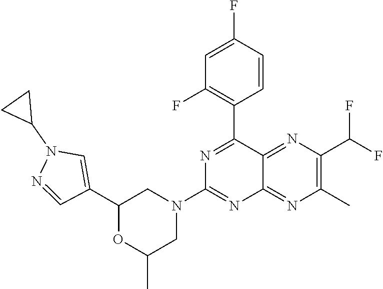 [SMILES: Cc1nc2nc(N3CC(C)OC(c4cnn(C5CC5)c4)C3)nc(-c3ccc(F)cc3F)c2nc1C(F)F] | I-1613 |
| 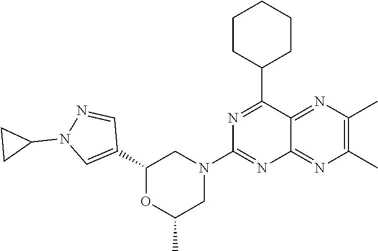 [SMILES: Cc1nc2nc(N3C[C@@H](c4cnn(C5CC5)c4)O[C@@H](C)C3)nc(C3CCCCC3)c2nc1C] | I-1614 |
| 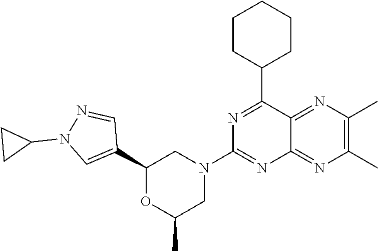 [SMILES: Cc1nc2nc(N3C[C@@H](C)O[C@@H](c4cnn(C5CC5)c4)C3)nc(C3CCCCC3)c2nc1C] | I-1615 |
| 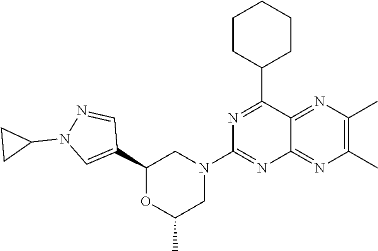 [SMILES: Cc1nc2nc(N3C[C@H](C)O[C@@H](c4cnn(C5CC5)c4)C3)nc(C3CCCCC3)c2nc1C] | I-1616 |
| 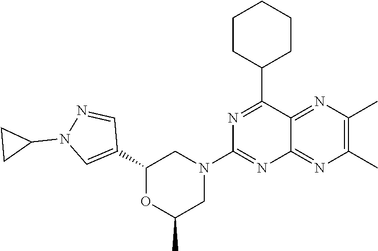 [SMILES: Cc1nc2nc(N3C[C@@H](C)O[C@H](c4cnn(C5CC5)c4)C3)nc(C3CCCCC3)c2nc1C] | I-1617 |
| 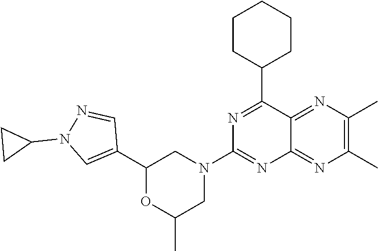 [SMILES: Cc1nc2nc(N3CC(C)OC(c4cnn(C5CC5)c4)C3)nc(C3CCCCC3)c2nc1C] | I-1618 |
| 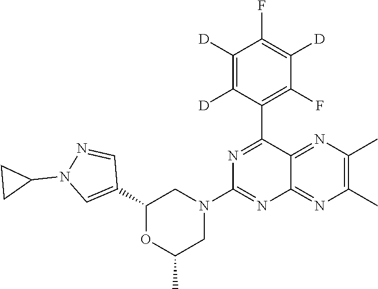 [SMILES: [2H]c1c([2H])c(-c2nc(N3C[C@@H](c4cnn(C5CC5)c4)O[C@@H](C)C3)nc3nc(C)c(C)nc23)c(F)c([2H])c1F] | I-1619 |
| 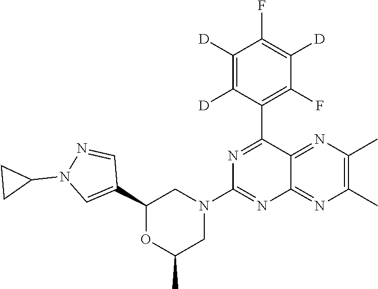 [SMILES: [2H]c1c([2H])c(-c2nc(N3C[C@@H](C)O[C@@H](c4cnn(C5CC5)c4)C3)nc3nc(C)c(C)nc23)c(F)c([2H])c1F] | I-1620 |
| 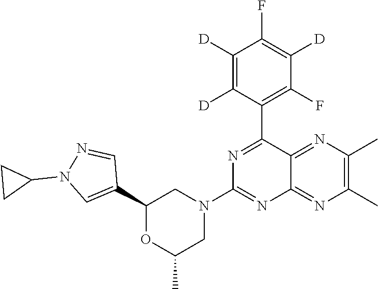 [SMILES: [2H]c1c([2H])c(-c2nc(N3C[C@H](C)O[C@@H](c4cnn(C5CC5)c4)C3)nc3nc(C)c(C)nc23)c(F)c([2H])c1F] | I-1621 |
| 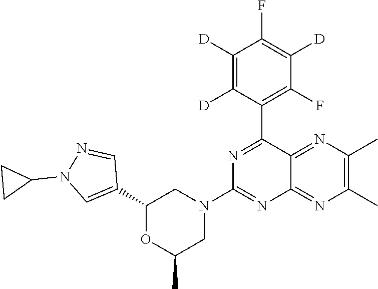 [SMILES: [2H]c1c([2H])c(-c2nc(N3C[C@@H](C)O[C@H](c4cnn(C5CC5)c4)C3)nc3nc(C)c(C)nc23)c(F)c([2H])c1F] | I-1622 |
| 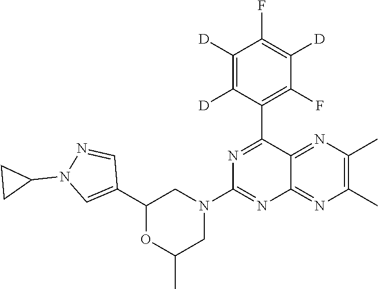 [SMILES: [2H]c1c([2H])c(-c2nc(N3CC(C)OC(c4cnn(C5CC5)c4)C3)nc3nc(C)c(C)nc23)c(F)c([2H])c1F] | I-1623 |
| 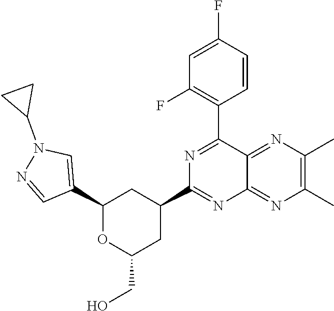 [SMILES: Cc1nc2nc([C@H]3C[C@H](CO)O[C@@H](c4cnn(C5CC5)c4)C3)nc(-c3ccc(F)cc3F)c2nc1C] | I-1624 |
| 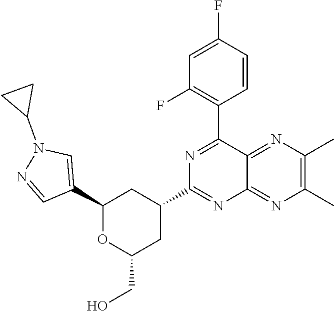 [SMILES: Cc1nc2nc([C@@H]3C[C@H](CO)O[C@@H](c4cnn(C5CC5)c4)C3)nc(-c3ccc(F)cc3F)c2nc1C] | I-1625 |
| 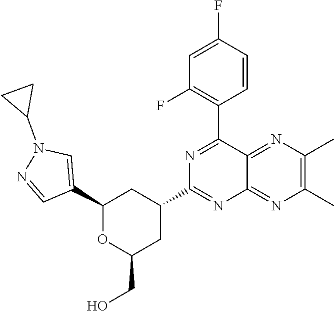 [SMILES: Cc1nc2nc([C@@H]3C[C@@H](CO)O[C@@H](c4cnn(C5CC5)c4)C3)nc(-c3ccc(F)cc3F)c2nc1C] | I-1626 |
| 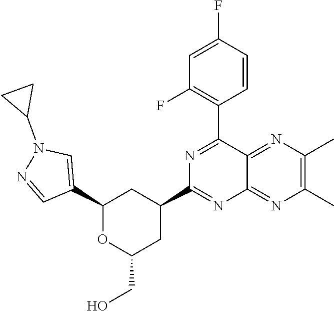 [SMILES: Cc1nc2nc([C@H]3C[C@H](CO)O[C@@H](c4cnn(C5CC5)c4)C3)nc(-c3ccc(F)cc3F)c2nc1C] | I-1627 |
| 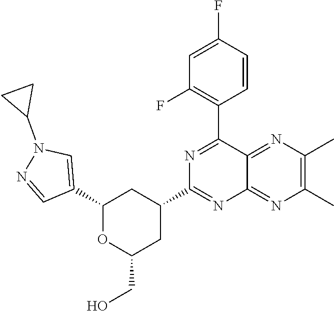 [SMILES: Cc1nc2nc([C@@H]3C[C@H](CO)O[C@H](c4cnn(C5CC5)c4)C3)nc(-c3ccc(F)cc3F)c2nc1C] | I-1628 |
| 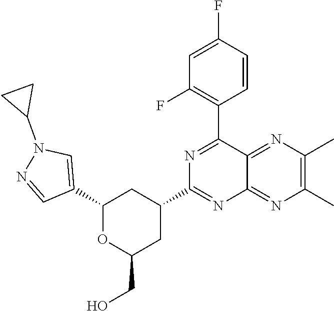 [SMILES: Cc1nc2nc([C@@H]3C[C@@H](CO)O[C@H](c4cnn(C5CC5)c4)C3)nc(-c3ccc(F)cc3F)c2nc1C] | I-1629 |
| 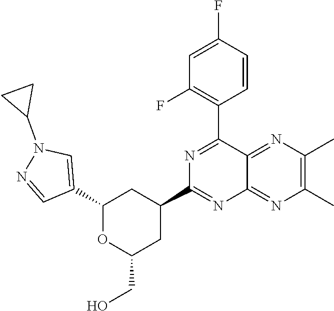 [SMILES: Cc1nc2nc([C@H]3C[C@H](CO)O[C@H](c4cnn(C5CC5)c4)C3)nc(-c3ccc(F)cc3F)c2nc1C] | I-1630 |
| 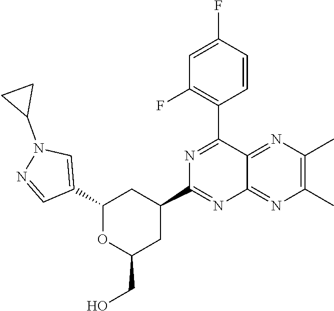 [SMILES: Cc1nc2nc([C@H]3C[C@@H](CO)O[C@H](c4cnn(C5CC5)c4)C3)nc(-c3ccc(F)cc3F)c2nc1C] | I-1631 |
| 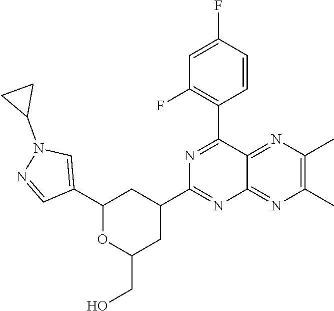 [SMILES: Cc1nc2nc(C3CC(CO)OC(c4cnn(C5CC5)c4)C3)nc(-c3ccc(F)cc3F)c2nc1C] | I-1632 |
| 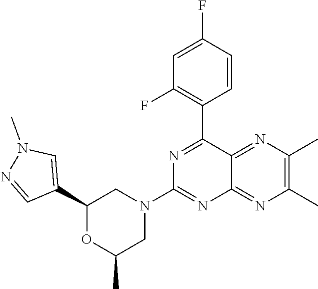 [SMILES: Cc1nc2nc(N3C[C@@H](C)O[C@@H](c4cnn(C)c4)C3)nc(-c3ccc(F)cc3F)c2nc1C] | I-1633 |
| 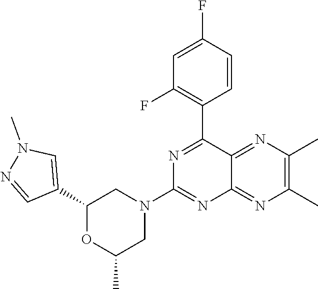 [SMILES: Cc1nc2nc(N3C[C@@H](c4cnn(C)c4)O[C@@H](C)C3)nc(-c3ccc(F)cc3F)c2nc1C] | I-1634 |
| 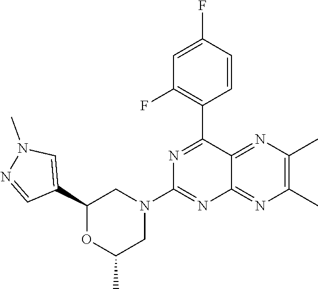 [SMILES: Cc1nc2nc(N3C[C@H](C)O[C@@H](c4cnn(C)c4)C3)nc(-c3ccc(F)cc3F)c2nc1C] | I-1635 |
| 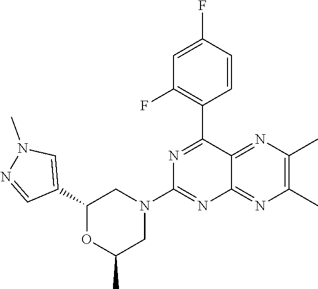 [SMILES: Cc1nc2nc(N3C[C@@H](C)O[C@H](c4cnn(C)c4)C3)nc(-c3ccc(F)cc3F)c2nc1C] | I-1636 |
| 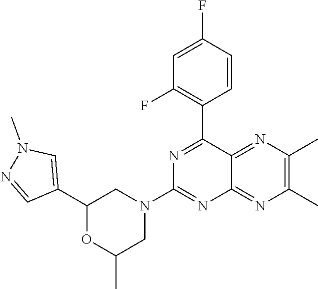 [SMILES: Cc1nc2nc(N3CC(C)OC(c4cnn(C)c4)C3)nc(-c3ccc(F)cc3F)c2nc1C] | I-1637 |
| 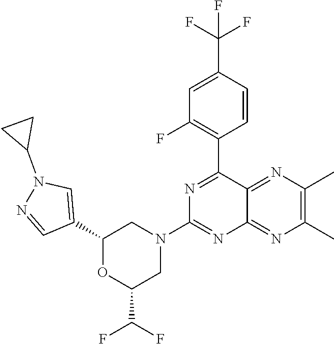 [SMILES: Cc1nc2nc(N3C[C@@H](c4cnn(C5CC5)c4)O[C@@H](C(F)F)C3)nc(-c3ccc(C(F)(F)F)cc3F)c2nc1C] | I-1638 |
| 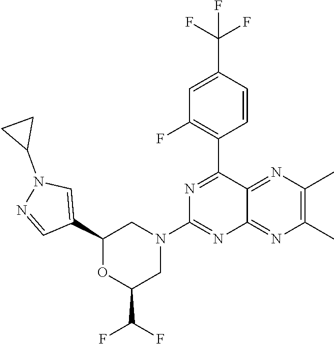 [SMILES: Cc1nc2nc(N3C[C@@H](C(F)F)O[C@@H](c4cnn(C5CC5)c4)C3)nc(-c3ccc(C(F)(F)F)cc3F)c2nc1C] | I-1639 |
| 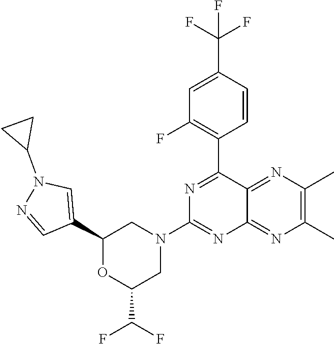 [SMILES: Cc1nc2nc(N3C[C@H](c4cnn(C5CC5)c4)O[C@@H](C(F)F)C3)nc(-c3ccc(C(F)(F)F)cc3F)c2nc1C] | I-1640 |
| 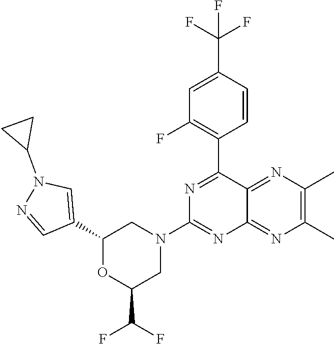 [SMILES: Cc1nc2nc(N3C[C@@H](c4cnn(C5CC5)c4)O[C@H](C(F)F)C3)nc(-c3ccc(C(F)(F)F)cc3F)c2nc1C] | I-1641 |
| 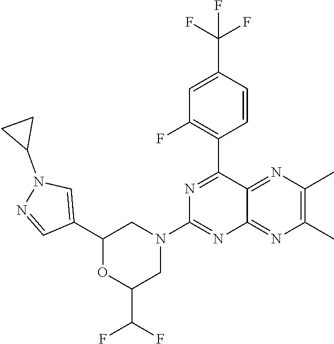 [SMILES: Cc1nc2nc(N3CC(c4cnn(C5CC5)c4)OC(C(F)F)C3)nc(-c3ccc(C(F)(F)F)cc3F)c2nc1C] | I-1642 |
| 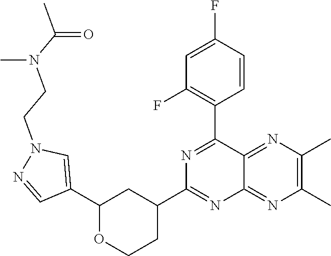 | I-1643 |
| 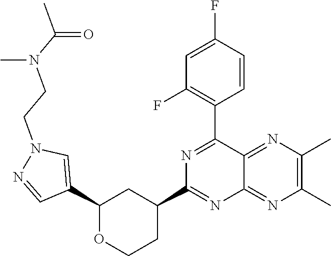 | I-1644 |
| 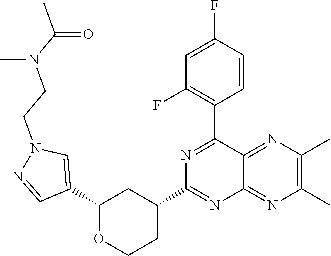 | I-1645 |
| 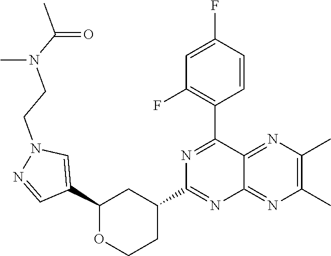 | I-1646 |
| 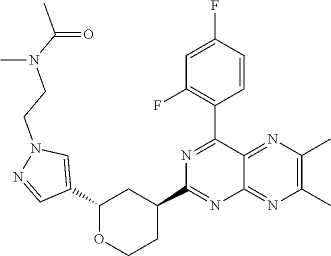 | I-1647 |
| 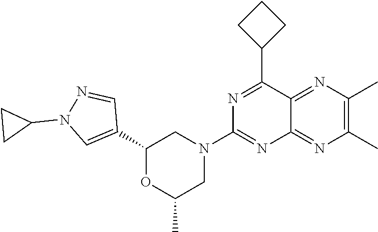 [SMILES: Cc1nc2nc(N3C[C@@H](c4cnn(C5CC5)c4)O[C@@H](C)C3)nc(C3CCC3)c2nc1C] | I-1648 |
| 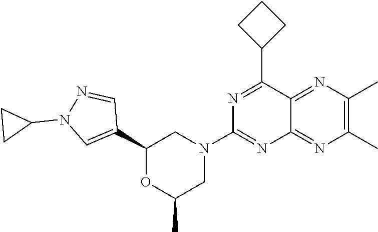 [SMILES: Cc1nc2nc(N3C[C@@H](C)O[C@@H](c4cnn(C5CC5)c4)C3)nc(C3CCC3)c2nc1C] | I-1649 |
| 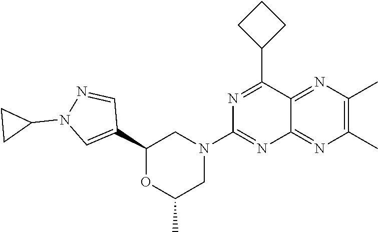 [SMILES: Cc1nc2nc(N3C[C@H](C)O[C@@H](c4cnn(C5CC5)c4)C3)nc(C3CCC3)c2nc1C] | I-1650 |
| 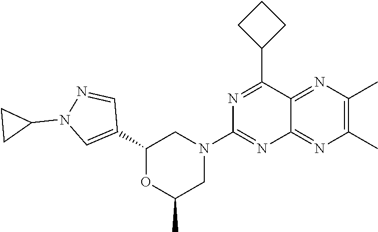 [SMILES: Cc1nc2nc(N3C[C@@H](C)O[C@H](c4cnn(C5CC5)c4)C3)nc(C3CCC3)c2nc1C] | I-1651 |
| 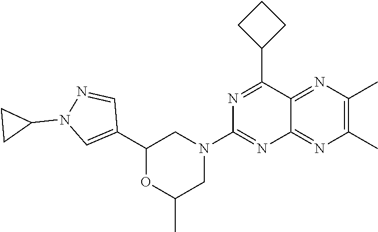 [SMILES: Cc1nc2nc(N3CC(C)OC(c4cnn(C5CC5)c4)C3)nc(C3CCC3)c2nc1C] | I-1652 |
| 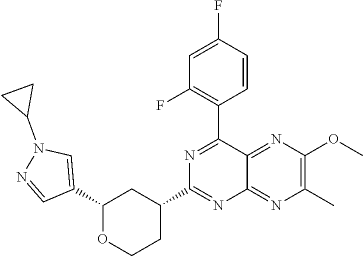 [SMILES: COc1nc2c(-c3ccc(F)cc3F)nc([C@@H]3CCO[C@H](c4cnn(C5CC5)c4)C3)nc2nc1C] | I-1653 |
| 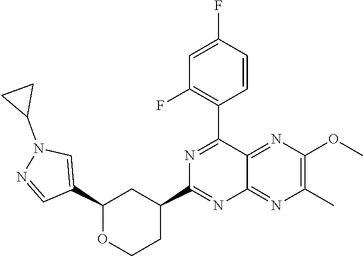 [SMILES: COc1nc2c(-c3ccc(F)cc3F)nc([C@H]3CCO[C@@H](c4cnn(C5CC5)c4)C3)nc2nc1C] | I-1654 |
| 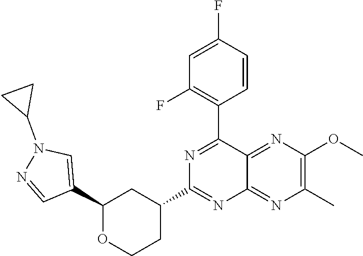 [SMILES: COc1nc2c(-c3ccc(F)cc3F)nc([C@@H]3CCO[C@@H](c4cnn(C5CC5)c4)C3)nc2nc1C] | I-1655 |
| 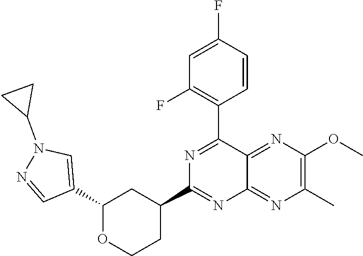 [SMILES: COc1nc2c(-c3ccc(F)cc3F)nc([C@H]3CCO[C@H](c4cnn(C5CC5)c4)C3)nc2nc1C] | I-1656 |
| 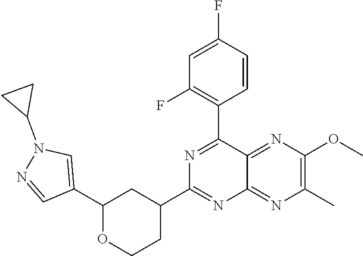 [SMILES: COc1nc2c(-c3ccc(F)cc3F)nc(C3CCOC(c4cnn(C5CC5)c4)C3)nc2nc1C] | I-1657 |
| 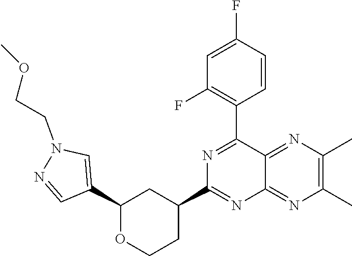 | I-1658 |
| 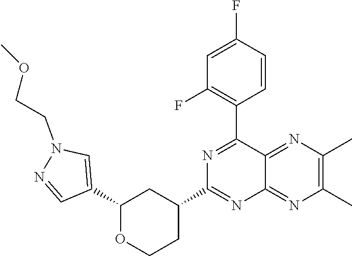 | I-1659 |
| 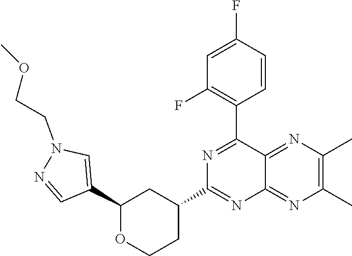 | I-1660 |
| 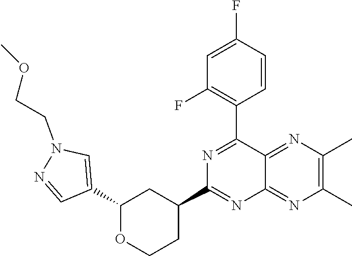 | I-1661 |
| 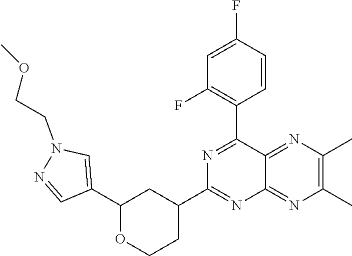 | I-1662 |
| 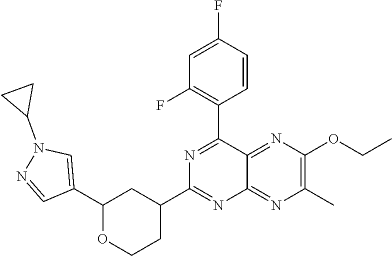 [SMILES: CCOc1nc2c(-c3ccc(F)cc3F)nc(C3CCOC(c4cnn(C5CC5)c4)C3)nc2nc1C] | I-1663 |
| 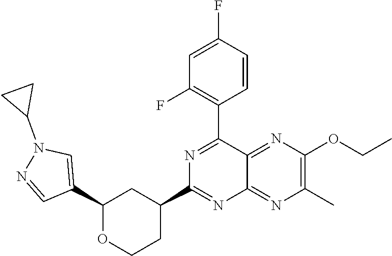 [SMILES: CCOc1nc2c(-c3ccc(F)cc3F)nc([C@H]3CCO[C@@H](c4cnn(C5CC5)c4)C3)nc2nc1C] | I-1664 |
| 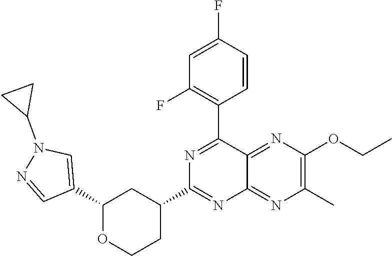 [SMILES: CCOc1nc2c(-c3ccc(F)cc3F)nc([C@@H]3CCO[C@H](c4cnn(C5CC5)c4)C3)nc2nc1C] | I-1665 |
| 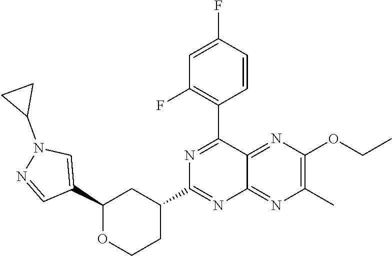 [SMILES: CCOc1nc2c(-c3ccc(F)cc3F)nc([C@@H]3CCO[C@@H](c4cnn(C5CC5)c4)C3)nc2nc1C] | I-1666 |
| 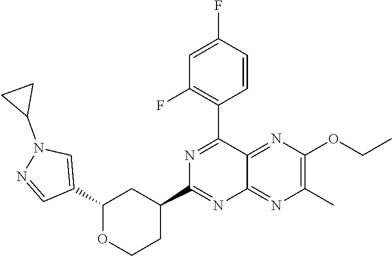 [SMILES: CCOc1nc2c(-c3ccc(F)cc3F)nc([C@H]3CCO[C@H](c4cnn(C5CC5)c4)C3)nc2nc1C] | I-1667 |
| 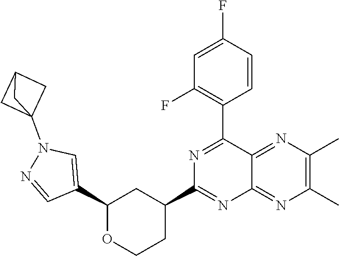 [SMILES: Cc1nc2nc([C@H]3CCO[C@@H](c4cnn(C56CC(C5)C6)c4)C3)nc(-c3ccc(F)cc3F)c2nc1C] | I-1668 |
| 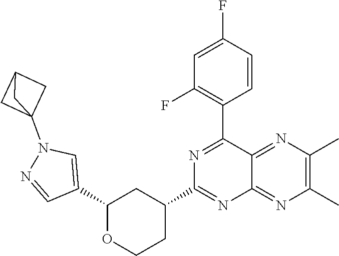 [SMILES: Cc1nc2nc([C@@H]3CCO[C@H](c4cnn(C56CC(C5)C6)c4)C3)nc(-c3ccc(F)cc3F)c2nc1C] | I-1669 |
| 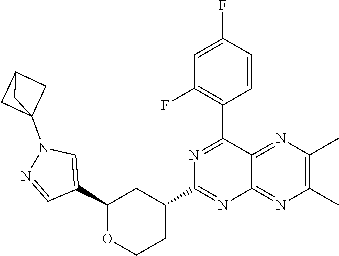 [SMILES: Cc1nc2nc([C@@H]3CCO[C@@H](c4cnn(C56CC(C5)C6)c4)C3)nc(-c3ccc(F)cc3F)c2nc1C] | I-1670 |
| 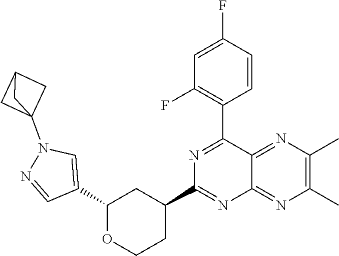 [SMILES: Cc1nc2nc([C@H]3CCO[C@H](c4cnn(C56CC(C5)C6)c4)C3)nc(-c3ccc(F)cc3F)c2nc1C] | I-1671 |
| 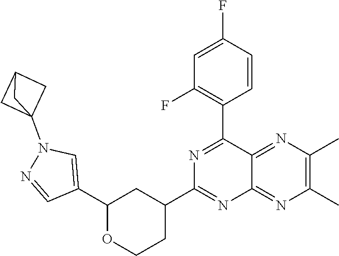 [SMILES: Cc1nc2nc(C3CCOC(c4cnn(C56CC(C5)C6)c4)C3)nc(-c3ccc(F)cc3F)c2nc1C] | I-1672 |
| 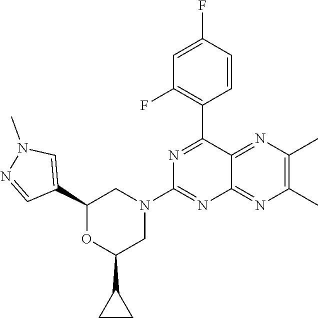 [SMILES: Cc1nc2nc(N3C[C@@H](C4CC4)O[C@@H](c4cnn(C)c4)C3)nc(-c3ccc(F)cc3F)c2nc1C] | I-1673 |
| 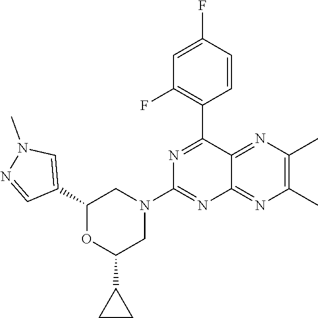 [SMILES: Cc1nc2nc(N3C[C@@H](c4cnn(C)c4)O[C@@H](C4CC4)C3)nc(-c3ccc(F)cc3F)c2nc1C] | I-1674 |
| 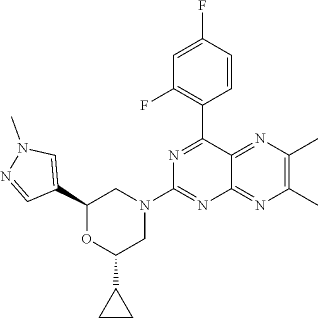 [SMILES: Cc1nc2nc(N3C[C@H](c4cnn(C)c4)O[C@@H](C4CC4)C3)nc(-c3ccc(F)cc3F)c2nc1C] | I-1675 |
| 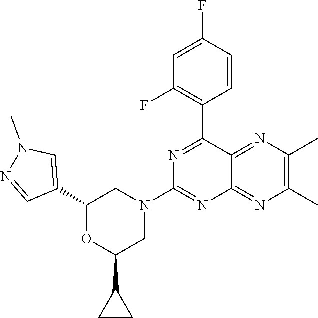 [SMILES: Cc1nc2nc(N3C[C@@H](c4cnn(C)c4)O[C@H](C4CC4)C3)nc(-c3ccc(F)cc3F)c2nc1C] | I-1676 |
| 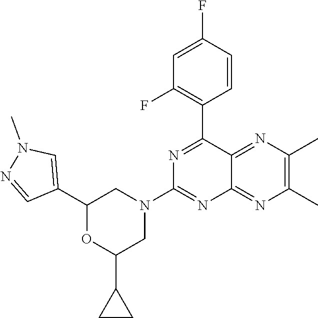 [SMILES: Cc1nc2nc(N3CC(c4cnn(C)c4)OC(C4CC4)C3)nc(-c3ccc(F)cc3F)c2nc1C] | I-1677 |
| 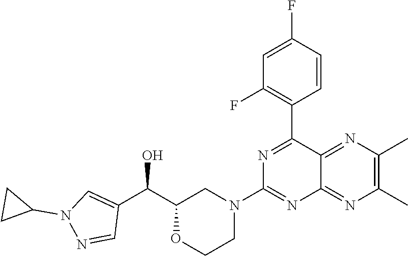 [SMILES: Cc1nc2nc(N3CCO[C@H]([C@H](O)c4cnn(C5CC5)c4)C3)nc(-c3ccc(F)cc3F)c2nc1C] | I-1678 |
| 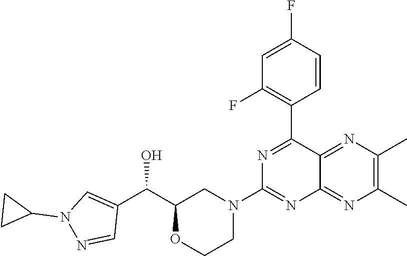 [SMILES: Cc1nc2nc(N3CCO[C@@H]([C@@H](O)c4cnn(C5CC5)c4)C3)nc(-c3ccc(F)cc3F)c2nc1C] | I-1679 |
| 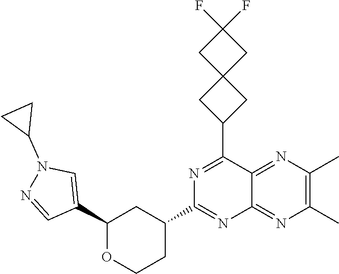 [SMILES: Cc1nc2nc([C@@H]3CCO[C@@H](c4cnn(C5CC5)c4)C3)nc(C3CC4(C3)CC(F)(F)C4)c2nc1C] | I-1680 |
| 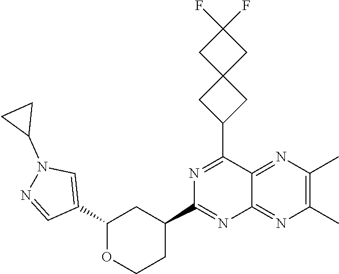 [SMILES: Cc1nc2nc([C@H]3CCO[C@H](c4cnn(C5CC5)c4)C3)nc(C3CC4(C3)CC(F)(F)C4)c2nc1C] | I-1681 |
| 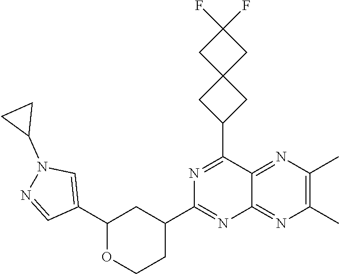 [SMILES: Cc1nc2nc(C3CCOC(c4cnn(C5CC5)c4)C3)nc(C3CC4(C3)CC(F)(F)C4)c2nc1C] | I-1682 |
| 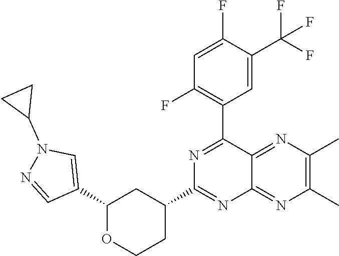 [SMILES: Cc1nc2nc([C@@H]3CCO[C@H](c4cnn(C5CC5)c4)C3)nc(-c3cc(C(F)(F)F)c(F)cc3F)c2nc1C] | I-1683 |
| 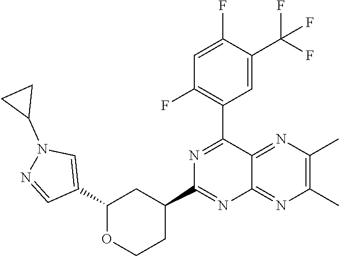 [SMILES: Cc1nc2nc([C@H]3CCO[C@H](c4cnn(C5CC5)c4)C3)nc(-c3cc(C(F)(F)F)c(F)cc3F)c2nc1C] | I-1684 |
| 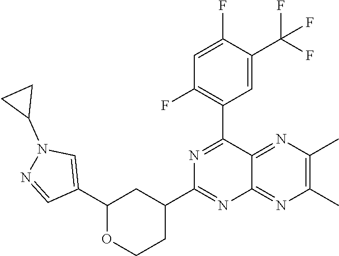 [SMILES: Cc1nc2nc(C3CCOC(c4cnn(C5CC5)c4)C3)nc(-c3cc(C(F)(F)F)c(F)cc3F)c2nc1C] | I-1685 |
| 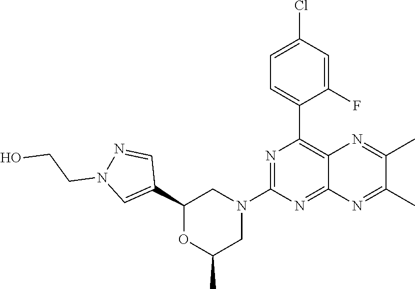 [SMILES: Cc1nc2nc(N3C[C@@H](C)O[C@@H](c4cnn(CCO)c4)C3)nc(-c3ccc(Cl)cc3F)c2nc1C] | I-1686 |
| 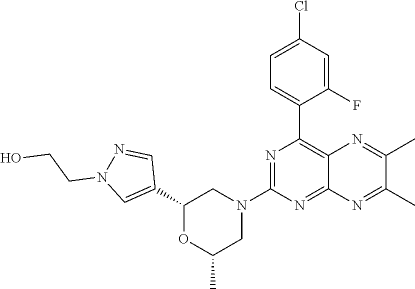 [SMILES: Cc1nc2nc(N3C[C@@H](c4cnn(CCO)c4)O[C@@H](C)C3)nc(-c3ccc(Cl)cc3F)c2nc1C] | I-1687 |
| 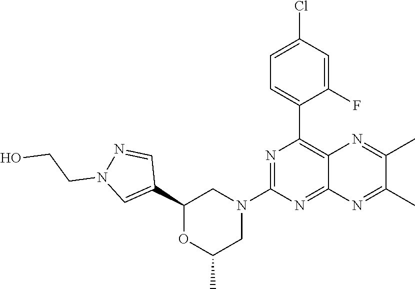 [SMILES: Cc1nc2nc(N3C[C@H](C)O[C@@H](c4cnn(CCO)c4)C3)nc(-c3ccc(Cl)cc3F)c2nc1C] | I-1688 |
| 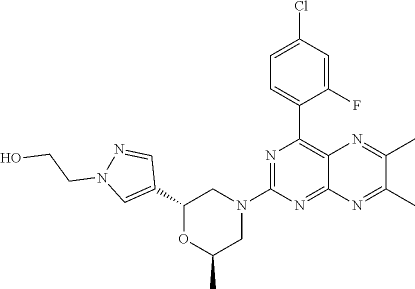 [SMILES: Cc1nc2nc(N3C[C@@H](C)O[C@H](c4cnn(CCO)c4)C3)nc(-c3ccc(Cl)cc3F)c2nc1C] | I-1689 |
| 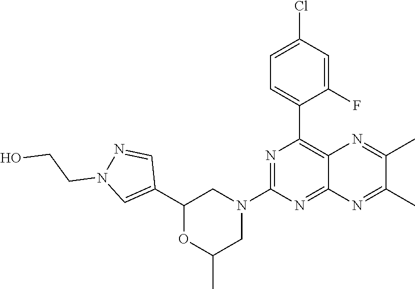 [SMILES: Cc1nc2nc(N3CC(C)OC(c4cnn(CCO)c4)C3)nc(-c3ccc(Cl)cc3F)c2nc1C] | I-1690 |
| 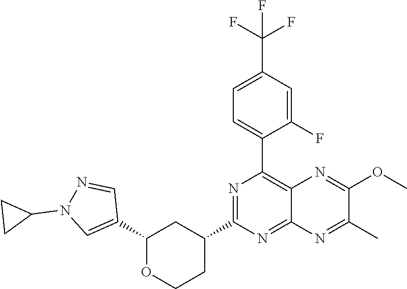 [SMILES: COc1nc2c(-c3ccc(C(F)(F)F)cc3F)nc([C@@H]3CCO[C@H](c4cnn(C5CC5)c4)C3)nc2nc1C] | I-1691 |
| 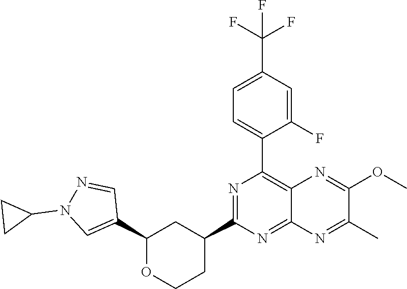 [SMILES: COc1nc2c(-c3ccc(C(F)(F)F)cc3F)nc([C@H]3CCO[C@@H](c4cnn(C5CC5)c4)C3)nc2nc1C] | I-1692 |
| 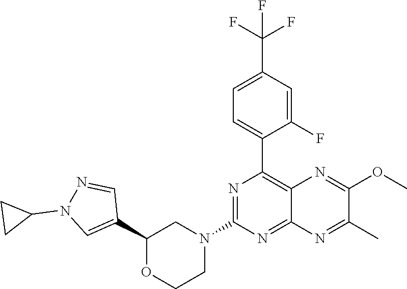 | I-1693 |
| 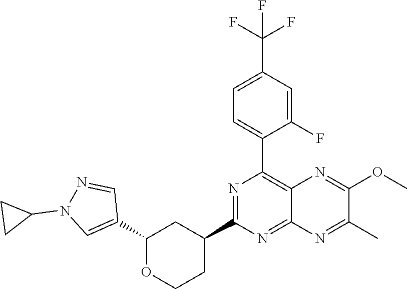 [SMILES: COc1nc2c(-c3ccc(C(F)(F)F)cc3F)nc([C@H]3CCO[C@H](c4cnn(C5CC5)c4)C3)nc2nc1C] | I-1694 |
| 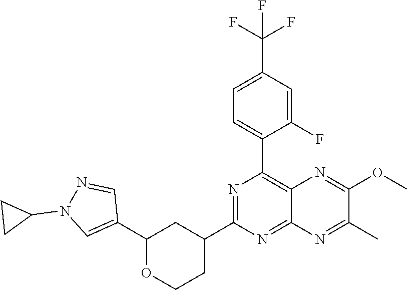 [SMILES: COc1nc2c(-c3ccc(C(F)(F)F)cc3F)nc(C3CCOC(c4cnn(C5CC5)c4)C3)nc2nc1C] | I-1695 |
| 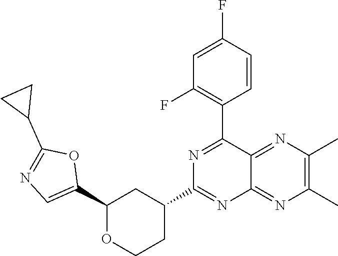 [SMILES: Cc1nc2nc([C@@H]3CCO[C@@H](c4cnc(C5CC5)o4)C3)nc(-c3ccc(F)cc3F)c2nc1C] | I-1696 |
| 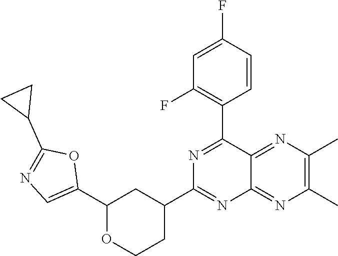 [SMILES: Cc1nc2nc(C3CCOC(c4cnc(C5CC5)o4)C3)nc(-c3ccc(F)cc3F)c2nc1C] | I-1697 |
| 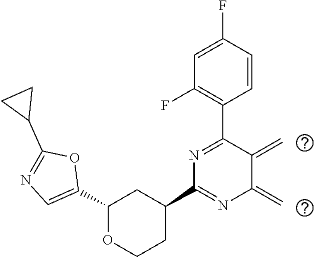 [SMILES: C=c1nc([C@H]2CCO[C@H](c3cnc(C4CC4)o3)C2)nc(-c2ccc(F)cc2F)c1=C] | I-1698 |
| 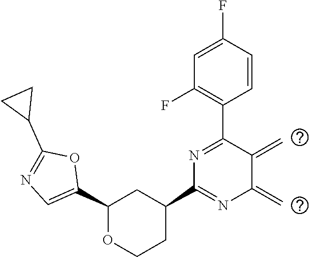 [SMILES: C=c1nc([C@H]2CCO[C@@H](c3cnc(C4CC4)o3)C2)nc(-c2ccc(F)cc2F)c1=C] | I-1699 |
| 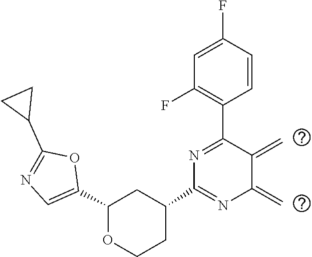 [SMILES: C=c1nc([C@@H]2CCO[C@H](c3cnc(C4CC4)o3)C2)nc(-c2ccc(F)cc2F)c1=C] | I-1700 |
| 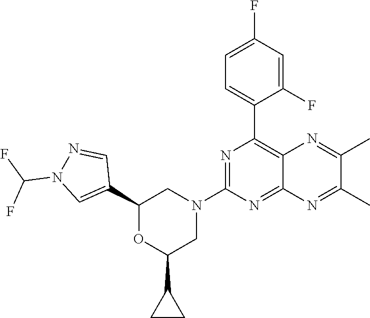 [SMILES: Cc1nc2nc(N3C[C@@H](C4CC4)O[C@@H](c4cnn(C(F)F)c4)C3)nc(-c3ccc(F)cc3F)c2nc1C] | I-1701 |
| 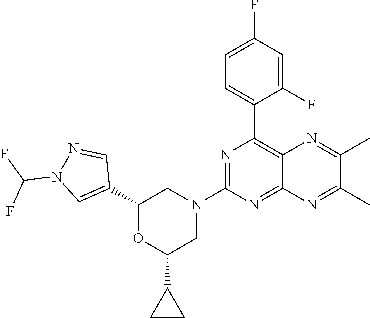 [SMILES: Cc1nc2nc(N3C[C@@H](c4cnn(C(F)F)c4)O[C@@H](C4CC4)C3)nc(-c3ccc(F)cc3F)c2nc1C] | I-1702 |
| 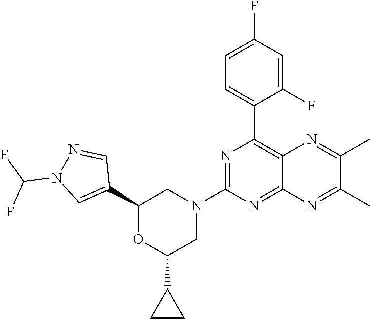 [SMILES: Cc1nc2nc(N3C[C@H](c4cnn(C(F)F)c4)O[C@@H](C4CC4)C3)nc(-c3ccc(F)cc3F)c2nc1C] | I-1703 |
| 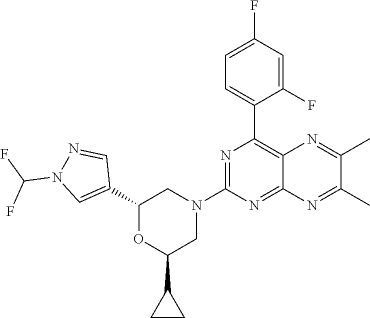 [SMILES: Cc1nc2nc(N3C[C@@H](c4cnn(C(F)F)c4)O[C@H](C4CC4)C3)nc(-c3ccc(F)cc3F)c2nc1C] | I-1704 |
| 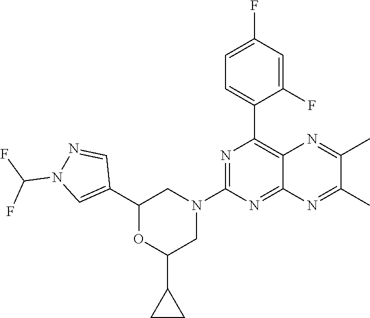 [SMILES: Cc1nc2nc(N3CC(c4cnn(C(F)F)c4)OC(C4CC4)C3)nc(-c3ccc(F)cc3F)c2nc1C] | I-1705 |
| 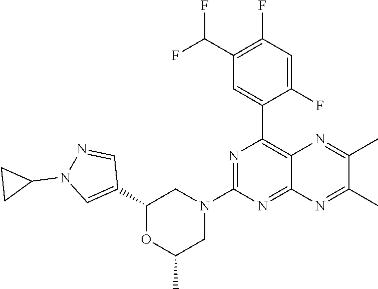 [SMILES: Cc1nc2nc(N3C[C@@H](c4cnn(C5CC5)c4)O[C@@H](C)C3)nc(-c3cc(C(F)F)c(F)cc3F)c2nc1C] | I-1706 |
| 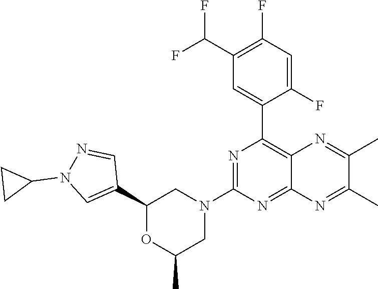 [SMILES: Cc1nc2nc(N3C[C@@H](C)O[C@@H](c4cnn(C5CC5)c4)C3)nc(-c3cc(C(F)F)c(F)cc3F)c2nc1C] | I-1707 |
| 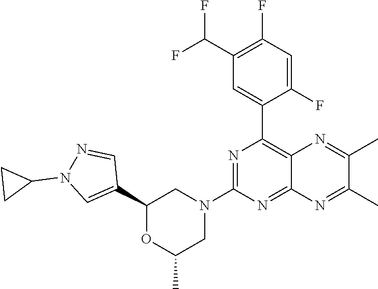 [SMILES: Cc1nc2nc(N3C[C@H](C)O[C@@H](c4cnn(C5CC5)c4)C3)nc(-c3cc(C(F)F)c(F)cc3F)c2nc1C] | I-1708 |
| 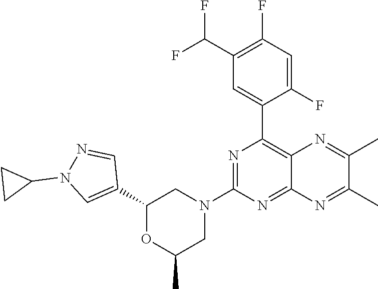 [SMILES: Cc1nc2nc(N3C[C@@H](C)O[C@H](c4cnn(C5CC5)c4)C3)nc(-c3cc(C(F)F)c(F)cc3F)c2nc1C] | I-1709 |
| 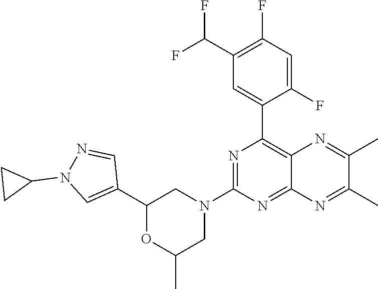 [SMILES: Cc1nc2nc(N3CC(C)OC(c4cnn(C5CC5)c4)C3)nc(-c3cc(C(F)F)c(F)cc3F)c2nc1C] | I-1710 |
| 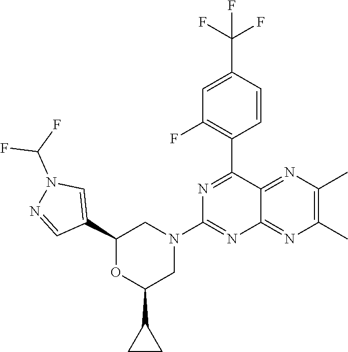 [SMILES: Cc1nc2nc(N3C[C@@H](C4CC4)O[C@@H](c4cnn(C(F)F)c4)C3)nc(-c3ccc(C(F)(F)F)cc3F)c2nc1C] | I-1711 |
| 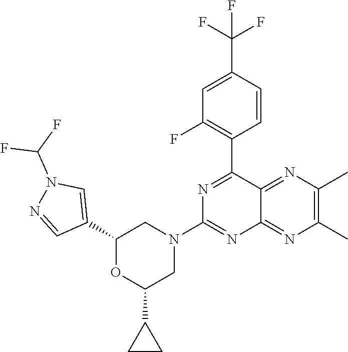 [SMILES: Cc1nc2nc(N3C[C@@H](c4cnn(C(F)F)c4)O[C@@H](C4CC4)C3)nc(-c3ccc(C(F)(F)F)cc3F)c2nc1C] | I-1712 |
| 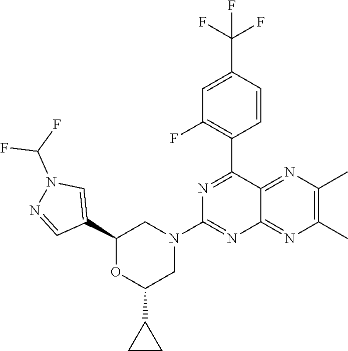 [SMILES: Cc1nc2nc(N3C[C@H](c4cnn(C(F)F)c4)O[C@@H](C4CC4)C3)nc(-c3ccc(C(F)(F)F)cc3F)c2nc1C] | I-1713 |
| 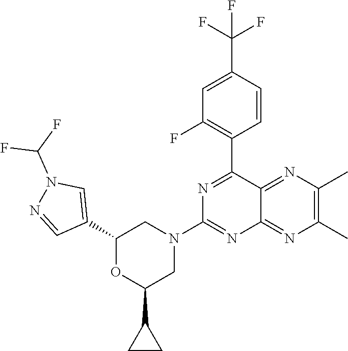 [SMILES: Cc1nc2nc(N3C[C@@H](c4cnn(C(F)F)c4)O[C@H](C4CC4)C3)nc(-c3ccc(C(F)(F)F)cc3F)c2nc1C] | I-1714 |
| 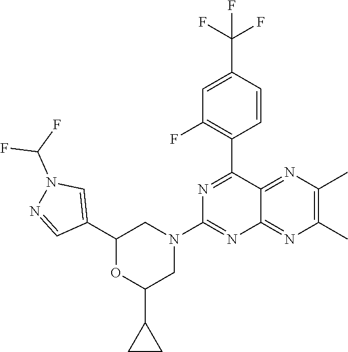 [SMILES: Cc1nc2nc(N3CC(c4cnn(C(F)F)c4)OC(C4CC4)C3)nc(-c3ccc(C(F)(F)F)cc3F)c2nc1C] | I-1715 |
| 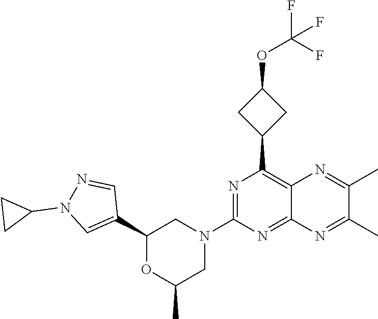 [SMILES: Cc1nc2nc(N3C[C@@H](C)O[C@@H](c4cnn(C5CC5)c4)C3)nc([C@H]3C[C@@H](OC(F)(F)F)C3)c2nc1C] | I-1716 |
| 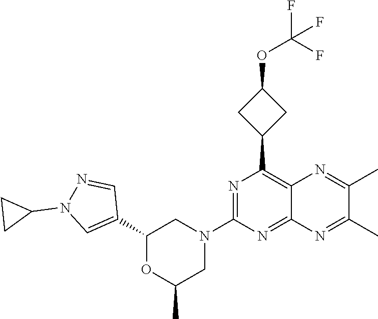 [SMILES: Cc1nc2nc(N3C[C@@H](C)O[C@H](c4cnn(C5CC5)c4)C3)nc([C@H]3C[C@@H](OC(F)(F)F)C3)c2nc1C] | I-1717 |
| 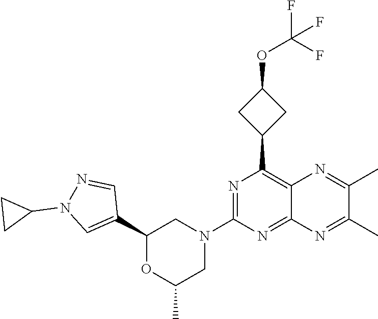 [SMILES: Cc1nc2nc(N3C[C@H](C)O[C@@H](c4cnn(C5CC5)c4)C3)nc([C@H]3C[C@@H](OC(F)(F)F)C3)c2nc1C] | I-1718 |
| 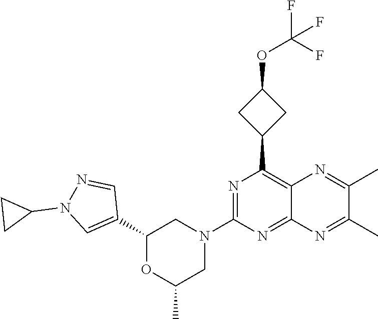 [SMILES: Cc1nc2nc(N3C[C@@H](c4cnn(C5CC5)c4)O[C@@H](C)C3)nc([C@H]3C[C@@H](OC(F)(F)F)C3)c2nc1C] | I-1719 |
| 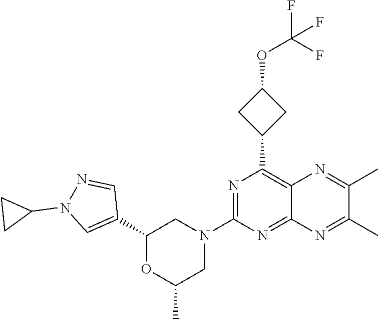 [SMILES: Cc1nc2nc(N3C[C@@H](c4cnn(C5CC5)c4)O[C@@H](C)C3)nc([C@H]3C[C@@H](OC(F)(F)F)C3)c2nc1C] | I-1720 |
| 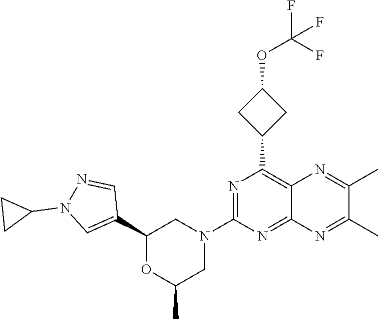 [SMILES: Cc1nc2nc(N3C[C@@H](C)O[C@@H](c4cnn(C5CC5)c4)C3)nc([C@H]3C[C@@H](OC(F)(F)F)C3)c2nc1C] | I-1721 |
| 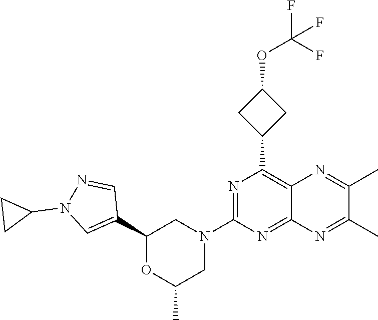 [SMILES: Cc1nc2nc(N3C[C@H](C)O[C@@H](c4cnn(C5CC5)c4)C3)nc([C@H]3C[C@@H](OC(F)(F)F)C3)c2nc1C] | I-1722 |
| 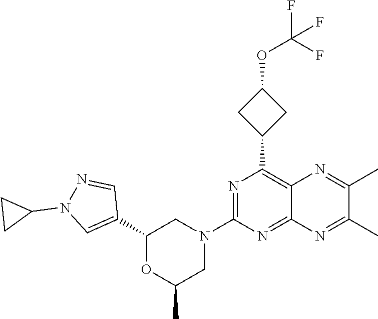 [SMILES: Cc1nc2nc(N3C[C@@H](C)O[C@H](c4cnn(C5CC5)c4)C3)nc([C@H]3C[C@@H](OC(F)(F)F)C3)c2nc1C] | I-1723 |
| 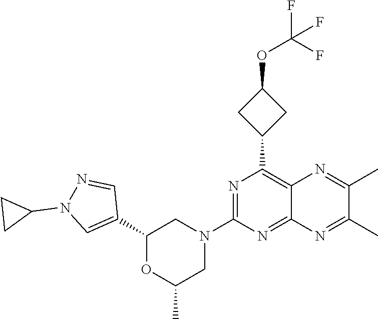 [SMILES: Cc1nc2nc(N3C[C@@H](c4cnn(C5CC5)c4)O[C@@H](C)C3)nc([C@H]3C[C@H](OC(F)(F)F)C3)c2nc1C] | I-1724 |
| 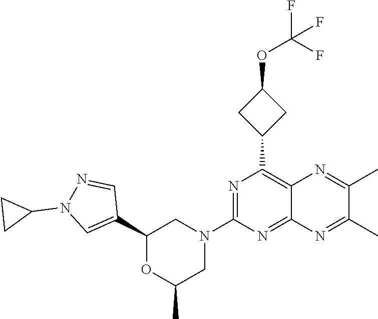 [SMILES: Cc1nc2nc(N3C[C@@H](C)O[C@@H](c4cnn(C5CC5)c4)C3)nc([C@H]3C[C@H](OC(F)(F)F)C3)c2nc1C] | I-1725 |
| 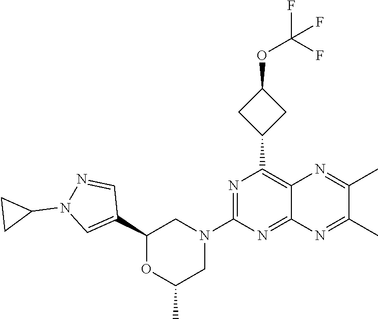 [SMILES: Cc1nc2nc(N3C[C@H](C)O[C@@H](c4cnn(C5CC5)c4)C3)nc([C@H]3C[C@H](OC(F)(F)F)C3)c2nc1C] | I-1726 |
| 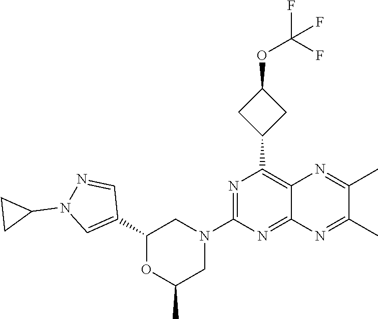 [SMILES: Cc1nc2nc(N3C[C@@H](C)O[C@H](c4cnn(C5CC5)c4)C3)nc([C@H]3C[C@H](OC(F)(F)F)C3)c2nc1C] | I-1727 |
| 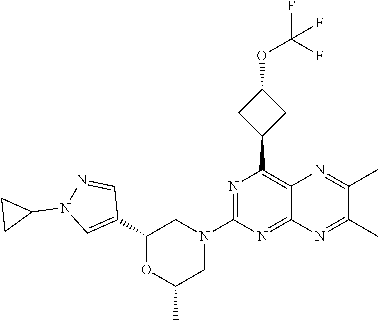 [SMILES: Cc1nc2nc(N3C[C@@H](c4cnn(C5CC5)c4)O[C@@H](C)C3)nc([C@H]3C[C@H](OC(F)(F)F)C3)c2nc1C] | I-1728 |
| 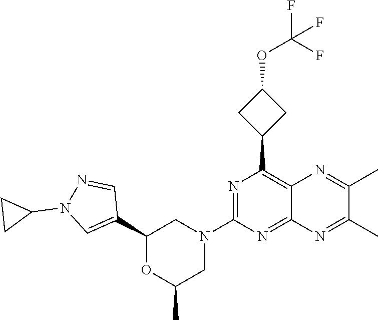 [SMILES: Cc1nc2nc(N3C[C@@H](C)O[C@@H](c4cnn(C5CC5)c4)C3)nc([C@H]3C[C@H](OC(F)(F)F)C3)c2nc1C] | I-1729 |
| 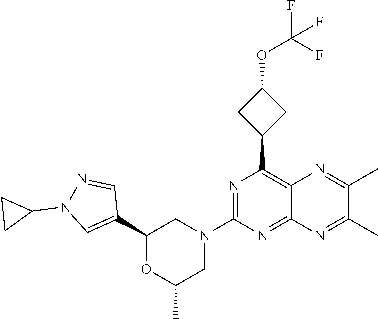 [SMILES: Cc1nc2nc(N3C[C@H](C)O[C@@H](c4cnn(C5CC5)c4)C3)nc([C@H]3C[C@H](OC(F)(F)F)C3)c2nc1C] | I-1730 |
| 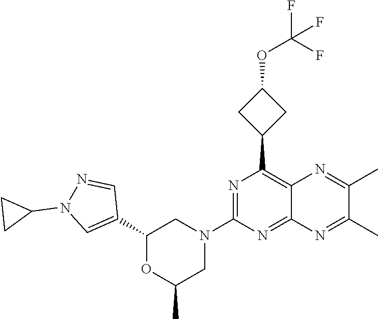 [SMILES: Cc1nc2nc(N3C[C@@H](C)O[C@H](c4cnn(C5CC5)c4)C3)nc([C@H]3C[C@H](OC(F)(F)F)C3)c2nc1C] | I-1731 |
| 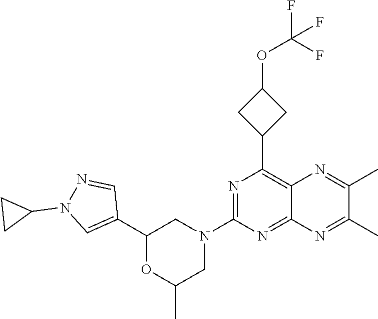 [SMILES: Cc1nc2nc(N3CC(C)OC(c4cnn(C5CC5)c4)C3)nc(C3CC(OC(F)(F)F)C3)c2nc1C] | I-1732 |
| 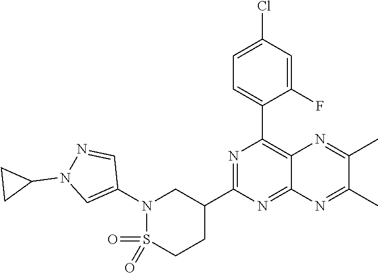 [SMILES: Cc1nc2nc(C3CCS(=O)(=O)N(c4cnn(C5CC5)c4)C3)nc(-c3ccc(Cl)cc3F)c2nc1C] | I-1733 |
| 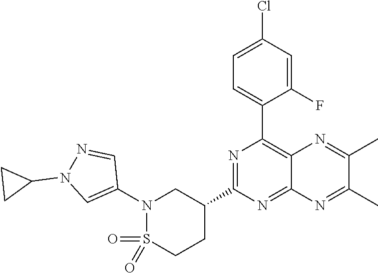 [SMILES: Cc1nc2nc([C@@H]3CCS(=O)(=O)N(c4cnn(C5CC5)c4)C3)nc(-c3ccc(Cl)cc3F)c2nc1C] | I-1734 |
| 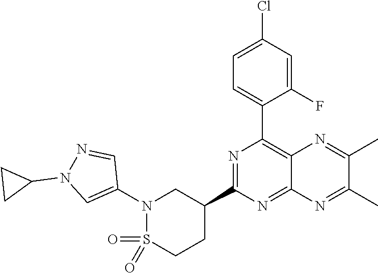 [SMILES: Cc1nc2nc([C@H]3CCS(=O)(=O)N(c4cnn(C5CC5)c4)C3)nc(-c3ccc(Cl)cc3F)c2nc1C] | I-1735 |
| 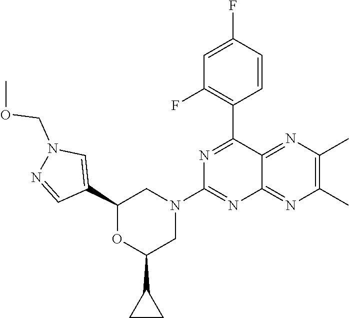 [SMILES: COCn1cc([C@H]2CN(c3nc(-c4ccc(F)cc4F)c4nc(C)c(C)nc4n3)C[C@@H](C3CC3)O2)cn1] | I-1736 |
| 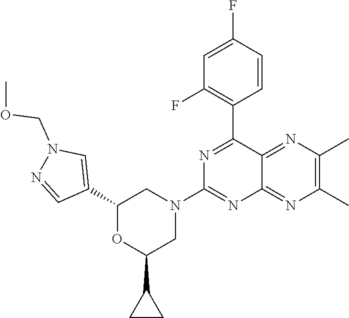 [SMILES: COCn1cc([C@@H]2CN(c3nc(-c4ccc(F)cc4F)c4nc(C)c(C)nc4n3)C[C@@H](C3CC3)O2)cn1] | I-1737 |
| 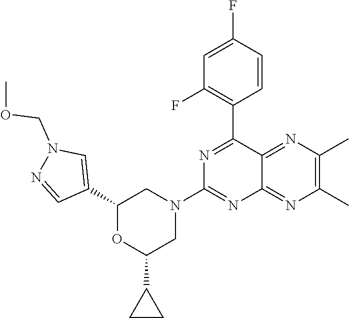 | I-1738 |
| 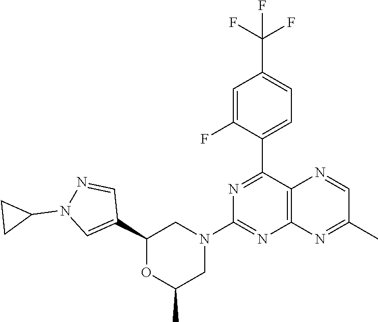 [SMILES: Cc1cnc2c(-c3ccc(C(F)(F)F)cc3F)nc(N3C[C@@H](C)O[C@@H](c4cnn(C5CC5)c4)C3)nc2n1] | I-1739 |
| 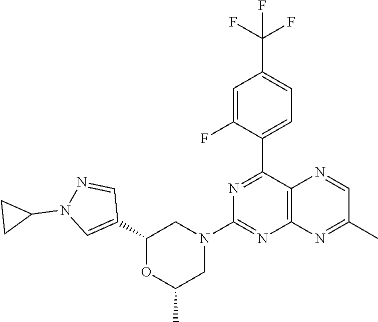 [SMILES: Cc1cnc2c(-c3ccc(C(F)(F)F)cc3F)nc(N3C[C@@H](c4cnn(C5CC5)c4)O[C@@H](C)C3)nc2n1] | I-1740 |
| 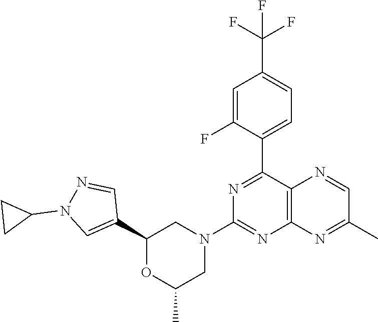 [SMILES: Cc1cnc2c(-c3ccc(C(F)(F)F)cc3F)nc(N3C[C@H](C)O[C@@H](c4cnn(C5CC5)c4)C3)nc2n1] | I-1741 |
| 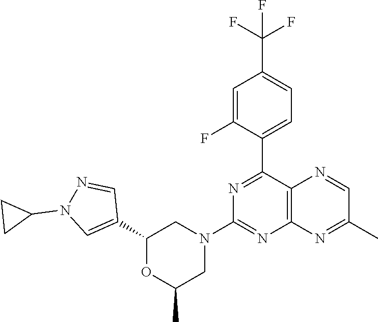 [SMILES: Cc1cnc2c(-c3ccc(C(F)(F)F)cc3F)nc(N3C[C@@H](C)O[C@H](c4cnn(C5CC5)c4)C3)nc2n1] | I-1742 |
| 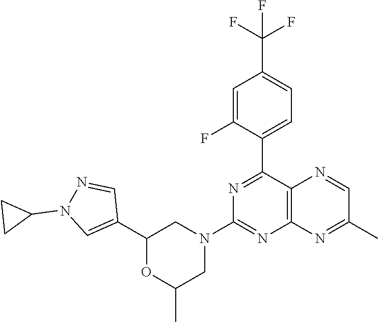 [SMILES: Cc1cnc2c(-c3ccc(C(F)(F)F)cc3F)nc(N3CC(C)OC(c4cnn(C5CC5)c4)C3)nc2n1] | I-1743 |
| 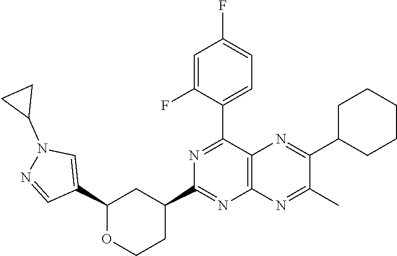 [SMILES: Cc1nc2nc([C@H]3CCO[C@@H](c4cnn(C5CC5)c4)C3)nc(-c3ccc(F)cc3F)c2nc1C1CCCCC1] | I-1744 |
| 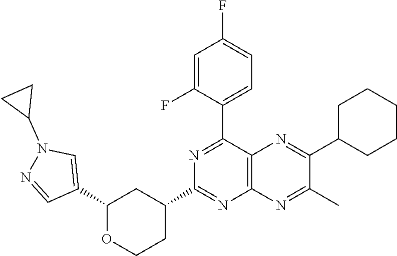 [SMILES: Cc1nc2nc([C@@H]3CCO[C@H](c4cnn(C5CC5)c4)C3)nc(-c3ccc(F)cc3F)c2nc1C1CCCCC1] | I-1745 |
| 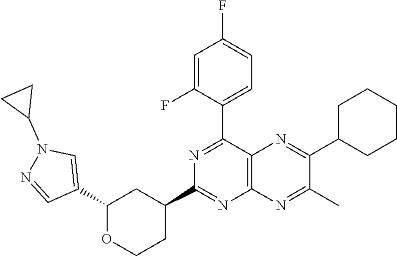 [SMILES: Cc1nc2nc([C@H]3CCO[C@H](c4cnn(C5CC5)c4)C3)nc(-c3ccc(F)cc3F)c2nc1C1CCCCC1] | I-1746 |
| 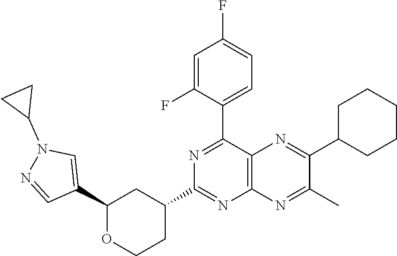 [SMILES: Cc1nc2nc([C@@H]3CCO[C@@H](c4cnn(C5CC5)c4)C3)nc(-c3ccc(F)cc3F)c2nc1C1CCCCC1] | I-1747 |
| 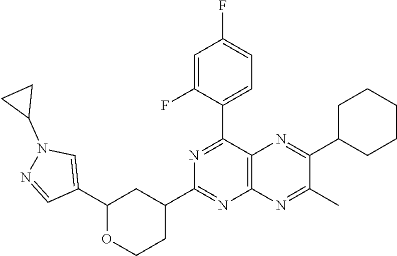 [SMILES: Cc1nc2nc(C3CCOC(c4cnn(C5CC5)c4)C3)nc(-c3ccc(F)cc3F)c2nc1C1CCCCC1] | I-1748 |
| 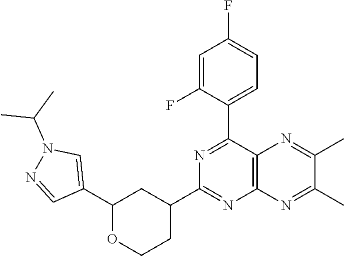 [SMILES: Cc1nc2nc(C3CCOC(c4cnn(C(C)C)c4)C3)nc(-c3ccc(F)cc3F)c2nc1C] | I-1749 |
| 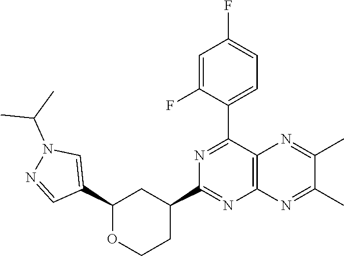 [SMILES: Cc1nc2nc([C@H]3CCO[C@@H](c4cnn(C(C)C)c4)C3)nc(-c3ccc(F)cc3F)c2nc1C] | I-1750 |
| 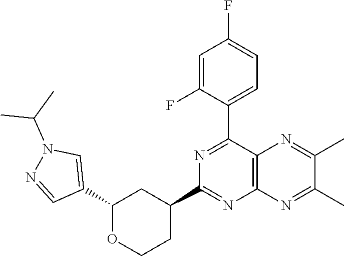 [SMILES: Cc1nc2nc([C@H]3CCO[C@H](c4cnn(C(C)C)c4)C3)nc(-c3ccc(F)cc3F)c2nc1C] | I-1751 |
| 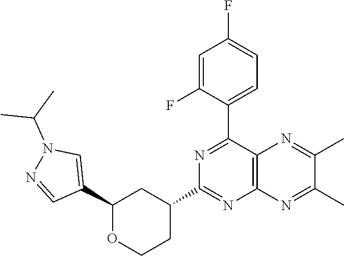 [SMILES: Cc1nc2nc([C@@H]3CCO[C@@H](c4cnn(C(C)C)c4)C3)nc(-c3ccc(F)cc3F)c2nc1C] | I-1752 |
| 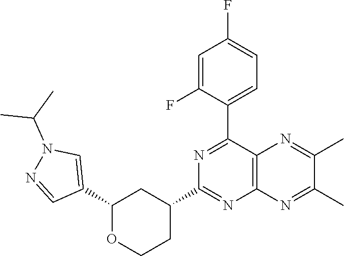 [SMILES: Cc1nc2nc([C@@H]3CCO[C@H](c4cnn(C(C)C)c4)C3)nc(-c3ccc(F)cc3F)c2nc1C] | I-1753 |

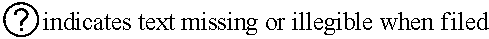

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by G D Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the invention provides a pharmaceutical composition comprising a compound of the present disclosure, or a tautomer thereof, or a pharmaceutically acceptable salt of said compound or said tautomer, and a pharmaceutically acceptable excipient.

In another aspect, the invention provides a compound of the present disclosure, or a tautomer thereof, or a pharmaceutically acceptable salt of said compound or said tautomer, or a pharmaceutical composition comprising said compound, or said tautomer, or said salt, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted: TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R⁴⁷H variant has been identified in genome-wide studies as being associated with increased risk for late-onset AD with an overall adjusted odds ratio (for populations of all ages) of 2.3, second only to the strong genetic association of ApoE to Alzheimer's. The R⁴⁷H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R⁴⁷H mutation are supportive of a novel loss-of-microglial barrier function for the carriers of the mutation, with the R⁴⁷H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et. al. 2019. A variety of animal models also suggest that Toll-Like Receptor (TLR) signaling is important in the pathogenesis of Rheumatoid Arthritis (RA) via persistent expression of pro-inflammatory cytokines by macrophages. Signaling through TREM2/DAP12 inhibits TLR responses by reducing MAPK (Erk1/2) activation, suggesting that TREM2 activation may act as a negative regulator of TLR driven RA pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the invention provides a compound of the present disclosure, or a tautomer thereof, or a pharmaceutically acceptable salt of said compound or said tautomer, or a pharmaceutical composition thereof for use in treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the invention provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the invention provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSF1R

CSF1R is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSF1R is composed of a highly glycosylated extracellular ligand-binding domain, a trans-membrane domain and an intracellular tyrosine-kinase domain. Binding of CSF-1 to CSF1R results in the formation of receptor homodimers and subsequent auto-phosphorylation of several tyrosine residues in the cytoplasmic domain, notably Syk. In the brain, CSF1R is predominantly expressed in microglial cells. It has been found that microglia in CSF1R+/−patients are depleted and show increased apoptosis (Oosterhof et al., 2018).

The present invention relates to the unexpected discovery that administration of a TREM2 agonist can rescue the loss of microglia in cells having mutations in CSF1R. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in CSF1R signaling caused by a decrease in the concentration of its ligand. In a 5×FAD murine Alzheimer's disease model of amyloid pathology, doses of a CSF1R inhibitor that almost completely eliminate microglia in the brains of wild-type animals show surviving microglia clustered around the amyloid plaques (Spangenberg et al, Nature Communications 2019). Plaque amyloid has been demonstrated in the past to be a ligand for TREM2, and it has been shown that microglial engagement with amyloid is dependent on TREM2 (Condello et al, Nat Comm., 2015). The present invention relates to the unexpected discovery that it is activation of TREM2 that rescued the microglia in the presence of the CSF1R inhibitor, and that this effect is also observed in patients suffering from loss of microglia due to CSF1R mutation. This discovery has not been previously taught or suggested in the available art.

To date, no prior study has shown that TREM2 agonism can rescue the loss of microglia in cells where mutations in the CSF1R kinase domain reduce CSF1R activity, rather than the presence of a CSF1R inhibitor or a deficiency in CSF1R ligand. Furthermore, no prior study has taught or suggested that reversal of the loss of microglia due to a CSF1R mutation through TREM2 agonism can be used to treat a disease or disorder caused by and/or associated with a CSF1R mutation.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSF1R, suggesting a reduced level of signaling on the macrophage colony-stimulating factor (M-CSF)/CSF1R axis (Rademakers et al, Nat Genet 2012; Konno et al, Neurology 2018). In one aspect, the present invention relates to the surprising discovery that activation of the TREM2 pathway can rescue the loss of microglia in CSF1R+/−ALSP patients, preventing microglia apoptosis, thereby treating the ALSP condition.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of Colony stimulating factor 1 receptor (CSF1R, also known as macrophage colony-stimulating factor receptor/M-CSFR, or cluster of differentiation 115/CD115).

In one aspect, the invention provides a compound of the present disclosure, or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of CSF1R.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the invention provides a method of treating or preventing a disease or disorder associated with dysfunction of CSF1R in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSF1R gene affecting the function of CSF1R. In some embodiments, the mutation in the CSF1R gene is a mutation that causes a decrease in CSF1R activity or a cessation of CSF1R activity. In some embodiments, the disease or disorder is caused by a heterozygous CSF1R mutation. In some embodiments, the disease or disorder is caused by a homozygous CSF1R mutation. In some embodiments, the disease or disorder is caused by a splice mutation in the csf1r gene. In some embodiments, the disease or disorder is caused by a missense mutation in the csf1r gene. In some embodiments, the disease or disorder is caused by a mutation in the catalytic kinase domain of CSF1R. In some embodiments, the disease or disorder is caused by a mutation in an immunoglobulin domain of CSF1R. In some embodiments, the disease or disorder is caused by a mutation in the ectodomain of CSF1R. In some embodiments, the disease or disorder is a disease or disorder resulting from a change (e.g. increase, decrease or cessation) in the activity of CSF1R. In some embodiments, the disease or disorder is a disease or disorder resulting from a decrease or cessation in the activity of CSF1R. CSF1R related activities that are changed in the disease or disorder include, but are not limited to: decrease or loss of microglia function; increased microglia apoptosis; decrease in Src signaling; decrease in Syk signaling; decreased microglial proliferation; decreased microglial response to cellular debris; decreased phagocytosis; and decreased release of cytokines in response to stimuli. In some embodiments, the disease or disorder is caused by a loss-of-function mutation in CSF1R. In some embodiments, the loss-of-function mutation results in a complete cessation of CSF1R function. In some embodiments, the loss-of-function mutation results in a partial loss of CSF1R function, or a decrease in CSF1R activity.

In another aspect, the invention provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSF1R. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF 1R, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82(5):813-827). It has been shown that severe microglia loss and damage is an early feature in patients with cerebral form of x-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018: 141; 2329-2342). These findings emphasize microglia/monocytes/macrophages as crucial therapeutic targets for preventing or stopping myelin destruction in patients with X-linked adrenoleukodystrophy.

The present invention relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present invention relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

To date, no prior study has shown that TREM2 agonism can rescue the loss of microglia in cells where mutations in the ABCD1 and a VLCFA increase is present. No prior study has taught or suggested that reversal of the loss of microglia due to an ABCD1 mutation through TREM2 agonism can be used to treat a disease or disorder caused by and/or associated with an ABCD1 mutation.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of ATP-binding cassette transporter 1 (ABCD1).

In one aspect, the invention provides a compound of the present disclosure, or a tautomer thereof, or a pharmaceutically acceptable salt of said compound or said tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of ABCD1.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the invention provides a method of treating or preventing a disease or disorder associated with dysfunction of ABCD1 in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the invention provides a method of treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (cALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, or a tautomer thereof, or a pharmaceutically acceptable salt of said compound or said tautomer, or a pharmaceutical composition thereof.

### Autism Spectrum Disorders

It has been found that TREM2 deficient mice exhibit symptoms reminiscent of autism spectrum disorders (ASDs) (Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present invention relates to the unexpected discovery that activation of TREM2, using a compound of the present invention, can rescue microglia in subjects suffering from an ASD. This discovery has not been previously taught or suggested in the available art.

In another aspect, the present invention provides a compound of the present disclosure, or a tautomer thereof, or a pharmaceutically acceptable salt of said compound or said tautomer, or a pharmaceutical composition thereof for use in treating autism or autism spectrum disorders.

In yet another aspect, the present invention provides a compound of the present disclosure, or a tautomer thereof, or a pharmaceutically acceptable salt of said compound or said tautomer, or a pharmaceutical composition thereof for use in the preparation of a medicament for treating autism or autism spectrum disorders.

In yet another aspect, the present invention provides a method of treating autism or autism spectrum disorders in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, or a pharmaceutically acceptable salt thereof with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, N Y, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example, the following is illustrative of tautomers of the compounds of Formula I, wherein Ring A together with the 6-membered ring system to which it is fused forms a bicyclic ring system of formula

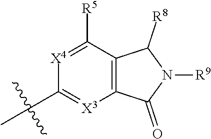

and wherein R⁹ is H:

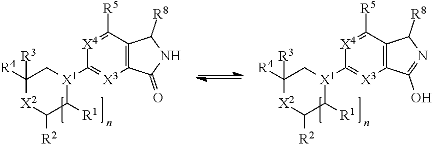

Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

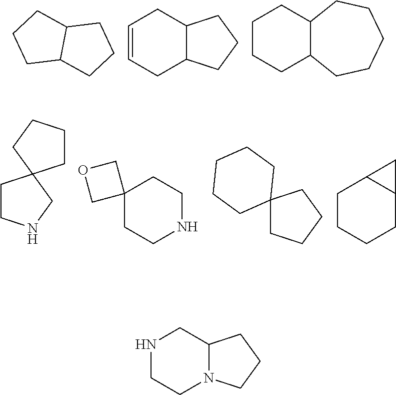

Exemplary bridged bicyclics include:

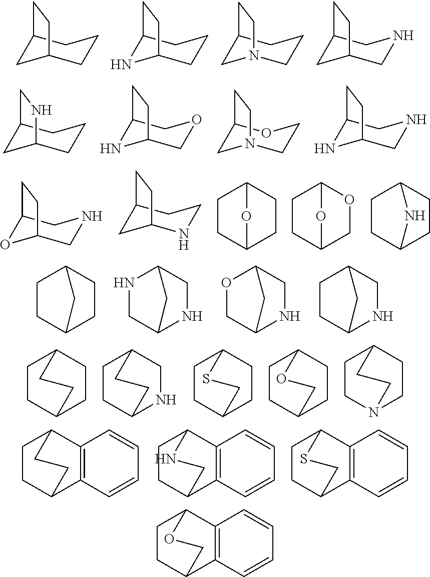

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁-6) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The terms “C₁₋₃alkyl,” “C₁₋₆alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₆alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR #, wherein R #represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “halogen” as used herein refers to —F, —Cl, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₆haloalkyl” refers to a C₁₋₆alkyl as defined herein, wherein one or more hydrogen atoms are substituted with a halogen. Representative examples of C₁₋₆haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃). Further, the term “C₁₋₆haloalkoxy” for example refers to a C₁₋₆alkoxy as defined herein, wherein one or more hydrogen atoms are substituted with a halogen. Representative examples of C₁₋₆haloalkoxy include, but are not limited to, —OCH₂F, —OCHF₂, —OCF₃, —OCHFCl, —OCH₂CF₃, —OCFHCF₃, —OCF₂CF₃, —OCH(CF₃)₂, —OCF(CHF₂)₂, and —OCH(CH₂F)(CF₃).

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1.3}]pentanyl,

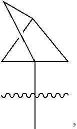 [SMILES: *C12CC(C1)C2]

tricyclo[2.1.1.0^{1.4}]hexanyl, tricyclo[3.1.1.0^{1.5}]hexanyl, and tricyclo[3.2.1.0^{1.5}]octanyl.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆C(O)OR^{∘}; —(CH₂)₀₋₆C(O)SR^{∘}; —(CH₂)₀₋₆C(O)OSiR^{∘}₃; —(CH₂)₀₋₆OC(OO)R^{∘}; —OC(O)(CH₂)₀₋₆SR^{∘},—(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)_{0- 6}C(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆S(O)₂R^{∘}; —(CH₂)₀₋₆S(O)₂OR^{∘}; —(CH₂)₀₋₆OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{•}, -(haloR^{•}), —OH, —OR*, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₄ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following scheme are the variables as defined for Formula I, unless otherwise noted. All starting materials are either commercially available, for example, from Merck Sigma-Aldrich Inc. and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein. As used below, Z is a leaving group, which can include but is not limited to, halogens (e.g. fluoride, chloride, bromide, iodide), sulfonates (e.g. mesylate, tosylate, benzenesulfonate, brosylate, nosylate, triflate), diazonium, and the like. As used below, in certain embodiments Y is an organometal coupling reagent group, which can include but are not limited to, boronic acids and esters, organotin and organozinc reagents.

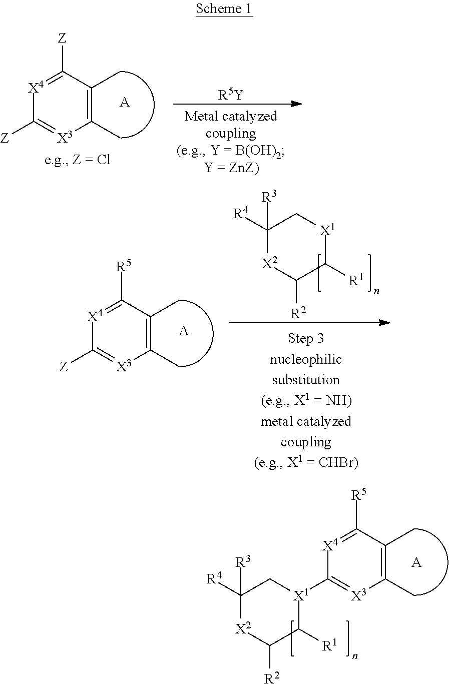

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### EXAMPLES

This section provides specific examples of compounds of Formula I and methods of making the same.

List of Abbreviations

| | |
|---|---|
| aq or aq. | aqueous |
| DCM | dichloromethane |
| DMAP | 4-dimethylaminopyridine |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | equivalent |
| ESI or ES | electrospray ionization |
| Et | ethyl |
| EtOAc or EA | ethyl acetate |
| g | gram(s) |
| h or hr | hour(s) |
| HPLC | high pressure liquid chromatography |
| iPr | isopropyl |
| iPr2NEt or DIPEA | N-ethyl diisopropylamine (Hunig's base) |
| LC MS, LCMS, | liquid chromatography mass spectroscopy |
| LC-MS or LC/MS | |
| m/z | mass divided by charge |
| Me | methyl |
| CH₃CN | acetonitrile |
| MeOH | methanol |
| mg | milligrams |
| min | minutes |
| mL | milliliters |
| MS | mass spectra |
| n-BuLi | n-butyllithium |
| NMR | nuclear magnetic resonance |
| PE | Petroleum ether |
| Ph | phenyl |
| RT or rt or r.t. | room temperature |
| RuPhos Pd G3 | (2-Dicyclohexylphosphino-2′,6′- |
| | diisopropoxy-1,1′-biphenyl)[2-(2′- |
| | amino-1,1′-biphenyl)]palladium (II) |
| | methanesulfonate |
| sat. | saturated |
| SFC | supercritical fluid chromatography |
| TEA or Et₃N | triethylamine |
| THF | tetrahydrofuran |
| Xantphos Pd G3 | [(4,5-Bis(diphenylphosphino)-9,9- |
| | dimethylxanthene)-2-(2′-amino-1,1′- |
| | biphenyl)]palladium(II) methanesulfonate |
| PE | Petroleum ether |

### General Analytical and Purification Methods

Provided in this section are descriptions of the general analytical and purification methods used to prepare the specific compounds provided herein.

### Chromatography:

Unless otherwise indicated, crude product-containing residues were purified by passing the crude material or concentrate through either a Biotage brand silica gel column pre-packed with flash silica (SiO₂) or reverse phase flash silica (C18) and eluting the product off the column with a solvent gradient as indicated. For example, a description of silica gel (0-40% EtOAc/hexane) means the product was obtained by elution from the column packed with silica using a solvent gradient of 0% to 40% EtOAc in hexanes.

### Preparative HPLC Method:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150×30 mm) or (b) Waters X-select CSH column (5 micron, C18, 100×30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

### Analytical HPLC Method:

Where so indicated, the compounds described herein were analyzed using an Agilent 1100 series instrument with DAD detector.

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2×25 cm, 5 μm or (b) Chiralpak AD-H 2×15 cm, 5 μm.

Some CP Analytical-SFC experiments were run on SFC Method Station (Thar, Waters) with the following conditions: Column temperature: 40° C., Mobile phase: CO₂/ Methanol (0.2% Methanol Ammonia)=Flow: 4.0 ml/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/ Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18(3) (150×25 mm, 10); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

### Mass Spectra (MS)

Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### Compound Names

The compounds disclosed and described herein have been named using the IUPAC naming function of ChemDraw Professional 17.0.

### Specific Examples

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example A1: Synthesis of Intermediates

### Method Int-1

### Intermediate 1: 5,7-Dichloro-2,3-Dimethylpyrido[3,4-b]Pyrazine

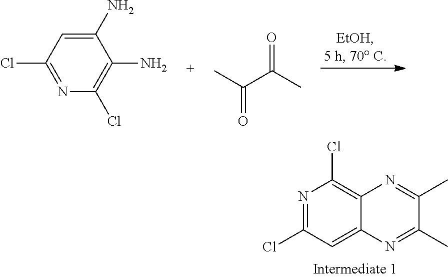

A 500 mL round bottom flask was charged with 3,4-diamino-2,6-dichloropyridine (27 g, 152 mmol) and 2,3-butanedione (15.99 mL, 182 mmol). EtOH (152 mL) was added to the flask and the mixture was heated to 70° C. After 5 h, the mixture was filtered through a fritted funnel and the eluent was concentrated to about 75 mL under reduced pressure. H₂O (150 mL) was added to the solution and the resulting solid was filtered off. The combined solid from both filtrations was washed with H₂O 3 times and was allowed to dry on the filter under air to afford 5,7-dichloro-2,3-dimethylpyrido[3,4-b]pyrazine as a light brown solid (34.5 g, 152 mmol). LC/MS (ESI⁺) m/z=228.0 [M+H]⁺¹H NMR (500 MHz, Chloroform-d) δ ppm 7.82 (s, 1H), 2.83 (s, 3H), 2.80 (s, 3H).

### Method Int-2

### Intermediate 2: 6,8-dichloro-2,3-dimethylpyrido[2,3-b]pyrazine

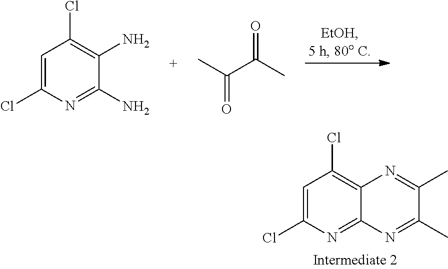

4,6-dichloropyridine-2,3-diamine (30 g, 169 mmol) and butane-2,3-dione (16.12 mL, 185 mmol) were combined in a 1 L round bottom flask. EtOH (600 mL) was added and the mixture was heated to 80° C. for 5 h. After cooling, the solvent was removed under reduced pressure. The resulting solid was triturated with diethyl ether and was filtered to afford 6,8-dichloro-2,3-dimethylpyrido[2,3-b]pyrazine as a light brown solid (36.5 g, 160 mmol). LC/MS (ESI⁺) m/z=228.0 [M+H]⁺¹H NMR (400 MHz, DMSO-d6): δ ppm 8.21 (s, 1H), 2.76 (s, 6H)

### Method Int-3

### Intermediate 3: 2,4-dichloro-6,7-dimethylpteridine

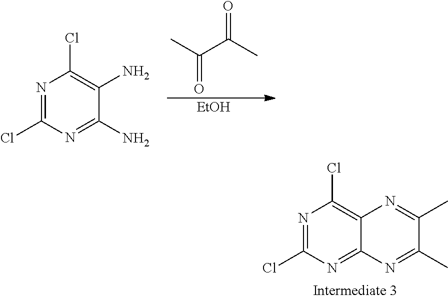

In a 100 mL round bottom flask 2,6-dichloropyrimidine-4,5-diamine (5 g, 27.9 mmol) and butane-2,3-dione (2.91 mL, 33.5 mmol) were combined in EtOH (27.9 mL) and the mixture was stirred at 30° C. for 18 h. After cooling, the solvent was removed under reduced pressure. The resulting solid was triturated with diethyl ether and filtered to afford 2,4-dichloro-6,7-dimethylpteridine (6.02 g, 26.3 mmol) as a light brown solid. LC/MS (ESI⁺) m/z=229.0 [M+H]⁺¹H NMR (500 MHz, Chloroform-d) δ ppm 2.88 (s, 3H), 2.87 (s, 3H).

TABLE1
Intermediate58 wasprepared following theprocedure described in Method Int-8, as follows:

| Int # | Structure | Name | Starting Material 1 | Starting Material 2 |
|---|---|---|---|---|
| 58 | 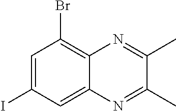 | 5-bromo-7-iodo- 2,3- dimethylquinoxaline | 3-bromo-5-iodo- benzene-1,2-diamine | butane-2,3-dione |

### Method Int-4

### Intermediate 4: 5,7-dichloro-2-methylpyrido[3,4-b]pyrazine

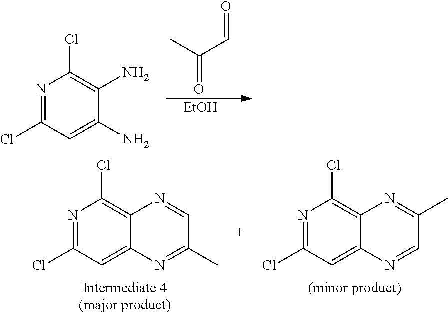

To a 50 mL round bottom flask were added 2,6-dichloropyridine-3,4-diamine (25 g, 140 mmol) and 2-oxopropanal (30.4 g, 169 mmol) in EtOH (250 mL). The reaction mixture was heated at 85° C. for 2 h. The reaction flask was cooled to room temperature. The mixture was diluted with H₂O and the resulting solids were filtered and washed with H₂O. The solid material was dissolved in DCM, dried over Na₂SO₄, filtered and concentrated under reduced pressure to furnish the reaction crude. This crude material was combined with 2,6-dichloropyridine-3,4-diamine from a second batch and the combined crude material was absorbed onto a plug of silica gel and purified by chromatography through a silica gel column, eluting with a gradient of 100% DCM, to provide 5,7-dichloro-2-methylpyrido[3,4-b]pyrazine (25.57 g, 119 mmol) as an off-white solid and 7.8 g of mixture of 2 isomers. Major isomer: LC/MS (ESI⁺) m/z=213.9 [M+H]⁺¹H NMR (400 MHz, DMSO-d6): δ ppm 9.06 (s, 1H), 8.11 (s, 1H), 2.79 (s, 3H). Minor isomer: LC/MS (ESI⁺) m/z=214.0 [M+H]⁺¹H NMR (400 MHz, DMSO-d6): δ ppm 9.16 (s, 1H), 8.20 (s, 1H), 2.79 (s, 3H).

### Method Int-5

### Intermediates 5 and 6: 5,7-dichloro-2,3-dimethyl-1,8-naphthyridine and 2,4-dichloro-7-ethyl-1,8-naphthyridine

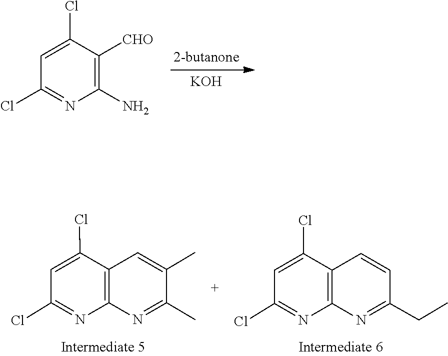

A screw-capped vial was charged with 2-amino-4,6-dichloronicotinaldehyde (0.5 g, 2.62 mmol) and methyl ethyl ketone (2.62 mL). To this solution was added KOH (0.147 g, 2.62 mmol). The reaction was stirred overnight at room temperature. H₂O was added and the aqueous phase was neutralized to a pH of 7 using 1N aqueous HCl. The aqueous phase was extracted with DCM. The organic phase was separated using a phase separator and was concentrated under reduced pressure. The crude material was purified by silica gel chromatography (0-10% MeOH (+1% NH₃) in DCM) to afford 5,7-dichloro-2,3-dimethyl-1,8-naphthyridine (0.284 g, 1.25 mmol, 47.7%). LC/MS (ESI⁺) m/z=227.0 [M+H]⁺ and 2,4-dichloro-7-ethyl-1,8-naphthyridine (0.18 g, 0.79 mmol) LC/MS (ESI⁺) m/z=227.0 [M+H]⁺.

### Method Int-6

### Intermediate 7: 5,7-dichloro-2-methyl-1,6-naphthyridine

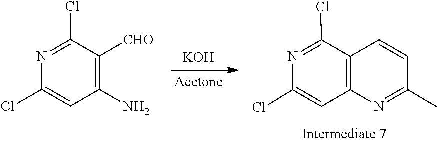

To a 50 mL vial were added 4-amino-2,6-dichloronicotinaldehyde (1.91 g, 10 mmol, JW Pharmlab) and KOH (0.84 g, 15.0 mmol) in acetone (10 mL). The reaction was stirred at rt for 30 min and a precipitate formed. The reaction mixture was diluted with EtOAc, dried, and concentrated. The crude material was purified via chromatography (0-30% EtOAc in DCM) to yield 1.65 g (71%) of 5,7-dichloro-2-methyl-1,6-naphthyridine as an off-white solid.

### Method Int-7

### Intermediate 8: 2,4-dichloro-7-methyl-1,8-naphthyridine

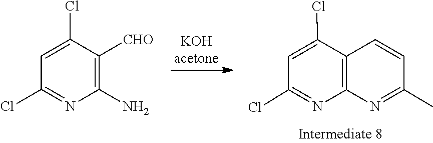

To a 50 mL vial were added 2-amino-4,6-dichloronicotinaldehyde (0.3507 g, 1.836 mmol) and acetone (1.836 mL). To this solution was added KOH (0.155 g, 2.75 mmol). The reaction was stirred at room temperature for 30 minutes. H₂O was added and the aqueous phase was extracted with DCM. The organic phase was separated using a phase separator and was concentrated under reduced pressure to afford 2,4-dichloro-7-methyl-1,8-naphthyridine (0.317 g, 1.49 mmol). LC/MS (ESI⁺) m/z=213.0 [M+H]⁺

### Method Int-8

### Intermediate 9: 2,4-dichloro-7-methylpteridine

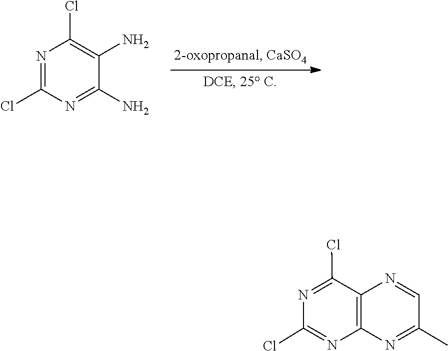

To a suspension of 2,6-dichloropyrimidine-4,5-diamine (5.00 g, 27.9 mmol) in DCE (250 mL) was added calcium sulfate (10.0 g, 73.5 mmol) followed by a dropwise addition of 2-oxopropanal (40% in water, 5.0 ml, 32.1 mmol). The reaction was stirred at 25° C. overnight then filtered through a plug of celite and evaporated under reduced pressure to afford the desired material as a light-yellow solid. (5.3 g, 88%). MS (m/z+): 215.0 [M+1]⁺, ¹H NMR (400 MHz, chloroform-d): 8.93 (1H, s), 2.91 (3H, s).

TABLE 2
Intermediate 10 was prepared following the procedure described in Method Int-8, as
follows:

| Int # | Structure | Name | Starting Material 1 | Starting Material 2 |
|---|---|---|---|---|
| 10 | 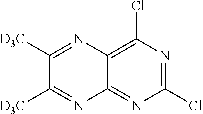 [SMILES: [2H]C([2H])([2H])c1nc2nc(Cl)nc(Cl)c2nc1C([2H])([2H])[2H]] | 2,4-dichloro-6,7- bis(methyl- d3)pteridine | 2,6- dichloropyrimidine- 4,5-diamine | butane-2,3-dione-d6 |

### Method Int-9

### Intermediate 11: 4-((2R,4S)-4-bromotetrahydro-2H-pyran-2-yl)-1-cyclopropyl-1H-pyrazole

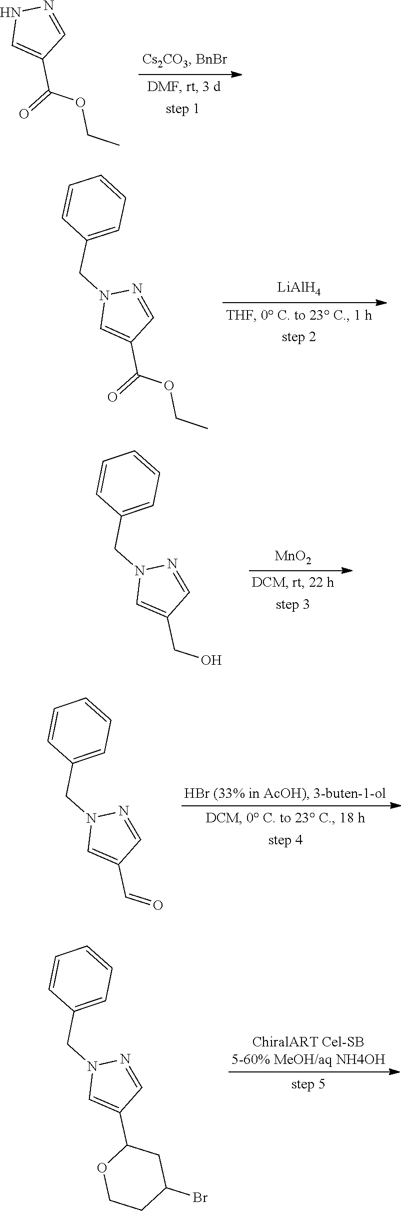

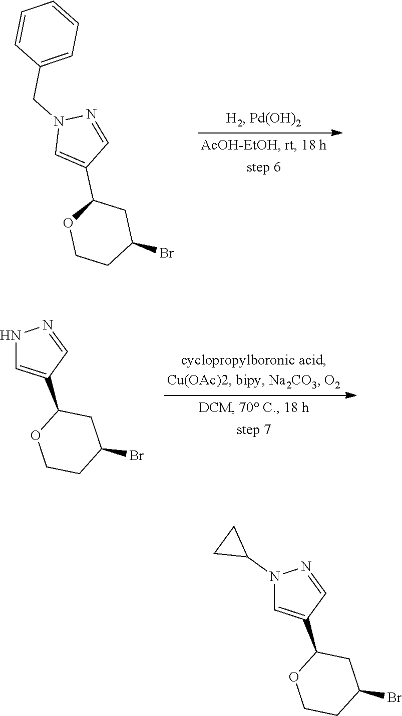

Step 1: To a solution of ethyl 1H-pyrazole-4-carboxylate (11.0 g, 78.5 mmol) in DMF (105 mL) was added cesium carbonate (51.2 g, 157 mmol), followed by benzyl bromide (9.3 mL, 78.4 mmol). The reaction was stirred at r.t. for 3 days. Water was added, and the product was extracted with EtOAc. The combined organic layers were washed several times with H₂O, then brine, dried over Na₂SO₄, filtered, and concentrated in vacuo to provide ethyl 1-benzyl-1H-pyrazole-4-carboxylate as a colorless syrup (16.7 g, 75.3 mmol, 96% yield). ¹H NMR (400 MHz, Chloroform-d) δ ppm 7.94 (s, 1H), 7.85 (s, 1H), 7.43-7.30 (m, 3H), 7.26-7.22 (m, 2H), 5.30 (s, 2H), 4.27 (q, J 7.1 Hz, 2H), 1.32 (t, J 7.1 Hz, 3H). LC/MS (ESI⁺) m/z=231.1 [M+H]⁺.

Step 2: To a solution of ethyl 1-benzyl-1H-pyrazole-4-carboxylate (6.37 g, 27.7 mmol) in THF (69 mL) at 0° C. was added lithium aluminum hydride (2M in THF, 28 mL, 56.0 mmol) slowly. The solution was warmed to r.t. and stirred for 1 hour. The reaction was cooled to 0° C., and water (2.2 mL) was added dropwise, followed by 1M NaOH (6.0 mL) and water (2.2 mL). The solid was filtered through celite, and the filter cake was rinsed with EtOAc. The filtrate was concentrated in vacuo to provide (1-benzyl-1H-pyrazol-4-yl)methanol (4.43 g, 22.8 mmol, 85% yield) as a colorless syrup. ¹H NMR (400 MHz, Chloroform-d) δ ppm 7.54 (s, 1H), 7.41-7.28 (m, 4H), 7.25-7.19 (m, 2H), 5.28 (s, 2H), 4.57 (s, 2H). LC/MS (ESI⁺) m/z=189.1 [M+H]⁺.

Step 3: To a solution of (1-benzyl-1H-pyrazol-4-yl)methanol (4.43 g, 22.8 mmol) in DCM (40 mL) was added activated manganese(IV) oxide (20.7 g, 235 mmol) portionwise. The mixture stirred overnight at r.t. The solid was filtered through celite and rinsed with DCM. The filtrate was concentrated in vacuo, and the crude material was purified by silica gel chromatography eluting with 0-40% EtOAc in hexanes to provide 1-benzyl-1H-pyrazole-4-carbaldehyde-1 (3.41 g, 18.3 mmol, 76% yield) as a colorless syrup. ¹H NMR (400 MHz, Chloroform-d) δ ppm 9.84 (s, 1H), 8.00 (s, 1H), 7.87 (s, 1H), 7.44-7.32 (m, 3H), 7.31-7.21 (m, 2H), 5.34 (s, 2H). LC/MS (ESI⁺) m/z=187.1 [M+H]⁺.

Step 4: To a solution of 1-benzyl-1H-pyrazole-4-carbaldehyde (3.05 g, 16.4 mmol) and 3-buten-1-ol (1.5 mL, 17.0 mmol) in DCM (41 mL) at 0° C. was added hydrobromic acid, 33% in acetic acid (8.1 mL, 49.1 mmol) dropwise. The solution was slowly warmed to r.t. overnight. The solution was then cooled to 0° C. and slowly quenched with saturated NaHCO₃ solution. The product was extracted with DCM. The combined organic layers were washed with brine, dried over Na₂SO₄, filtered, and concentrated in vacuo. The crude material was purified by silica gel chromatography eluting with 0-35% EtOAc in hexanes to provide 1-benzyl-4-(4-bromotetrahydro-2H-pyran-2-yl)-1H-pyrazole (4.13 g, 12.9 mmol, 75% yield) as a 1:1 mixture of cis/trans diastereomers. (¹H NMR reported as a 1:1 mixture of cis and trans.) ¹H NMR (400 MHz, Chloroform-d) δ ppm 7.50 (s, 2H), 7.39-7.27 (m, 8H), 7.24-7.19 (m, 4H), 5.26 (s, 4H), 4.90 (dd, J=9.8, 3.1 Hz, 1H), 4.76 (t, J 3.4 Hz, 1H), 4.33 (dd, J=11.4, 2.0 Hz, 1H), 4.21 (tt, J 11.8, 4.5 Hz, 1H), 4.13-4.01 (m, 2H), 3.92 (dd, J=12.3, 4.7 Hz, 1H), 3.54 (td, J=12.1, 2.3 Hz, 1H), 2.48 (dt, J 14.0, 2.8 Hz, 1H), 2.25-2.18 (m, 2H), 2.18-2.12 (m, 3H), 2.11-2.03 (m, 1H), 1.99-1.87 (m, 1H). LC/MS (ESI⁺) m/z=320.9 [M+H]⁺.

Step 5: The racemic product was purified by chiral SFC on a ChiralART Cel-SB column, 5 to 60% MeOH in aqueous NH₄OH solution to provide 1-benzyl-4-((2R,4S)-4-bromotetrahydro-2H-pyran-2-yl)-1H-pyrazole. ¹H NMR (400 MHz, Chloroform-d) δ ppm 7.50 (s, 1H), 7.44-7.28 (m, 4H), 7.22 (d, J 7.1 Hz, 2H), 5.26 (s, 2H), 4.33 (dd, J=11.4, 2.2 Hz, 1H), 4.26-4.13 (m, 1H), 4.12-3.95 (m, 1H), 3.54 (tt, J=12.1, 2.2 Hz, 1H), 2.48 (ddd, J=13.1, 4.5, 2.2 Hz, 1H), 2.27-2.18 (m, 1H), 2.11 (qd, J=11.9, 5.1 Hz, 2H). LC/MS (ESI⁺) m/z=321.0 [M+H]⁺.

Step 6: A solution 1-benzyl-4-((2R,4S)-4-bromotetrahydro-2H-pyran-2-yl)-1H-pyrazole (400 mg, 1.25 mmol) in EtOH (6.5 mL) and acetic acid (2.2 mL) was purged with argon via balloon and outlet for 10 minutes. Palladium hydroxide on carbon (70 mg, 0.25 mmol) was added quickly, and the solution was purged with argon via balloon and outlet for another 10 minutes. The argon balloon was replaced with a hydrogen balloon, and the reaction stirred at r.t. overnight. The catalyst was removed by filtration over celite and washed with ethanol several times. The filtrate was concentrated in vacuo. The crude material was purified by silica gel chromatography eluting with 30-100% EtOAc in hexanes to provide 4-((2R,4S)-4-bromotetrahydro-2H-pyran-2-yl)-1H-pyrazole (160 mg, 0.692 mmol, 56% yield) as a white solid. ¹H NMR (400 MHz, DMSO-d6) δ ppm 12.70 (s, 1H), 7.68 (s, 1H), 7.44 (s, 1H), 4.50 (td, J=12.0, 5.9 Hz, 1H), 4.37 (dd, J=11.1, 2.1 Hz, 1H), 3.91 (dd, J=11.8, 4.8 Hz, 1H), 3.51 (td, J=12.0, 2.1 Hz, 1H), 2.43 (dt, J 13.0, 2.6 Hz, 1H), 2.26-2.12 (m, 1H), 2.07-1.87 (m, 2H). LC/MS (ESI⁺) m/z=230.0 [M+H]⁺.

Step 7: To a solution of 4-((2R,4S)-4-bromotetrahydro-2H-pyran-2-yl)-1H-pyrazole (150 mg, 0.649 mmol) and cyclopropylboronic acid (112 mg, 1.30 mmol) in dichloroethane (4.3 mL) at 70° C. was added a mixture of copper(II) acetate (119 mg, 0.649 mmol) and 2,2′-dipyridyl (101 mg, 0.649 mmol) in one portion. The mixture was stirred at 70° C. overnight under oxygen atmosphere. The mixture was cooled to r.t., and saturated NaHCO₃ was added. The product was extracted with DCM, and the combined organic layers were washed with brine, dried over Na₂SO₄, filtered, and concentrated in vacuo. The crude product was purified by silica gel chromatography eluting with 10-60% EtOAc in hexanes to provide 4-((2R,4S)-4-bromotetrahydro-2H-pyran-2-yl)-1-cyclopropyl-1H-pyrazole (160 mg, 0.561 mmol, 86% yield) as a yellow oil. ¹H NMR (400 MHz, DMSO-d6) δ ppm 7.73 (s, 1H), 7.36 (s, 1H), 4.49 (tt, J 11.9, 4.4 Hz, 1H), 4.32 (dd, J=11.2, 2.0 Hz, 1H), 3.90 (ddd, J=11.8, 5.0, 1.8 Hz, 1H), 3.65 (tt, J 7.4, 3.9 Hz, 1H), 3.49 (td, J=12.0, 2.1 Hz, 1H), 2.41 (ddt, J 12.6, 4.3, 2.1 Hz, 1H), 2.17 (ddd, J=12.7, 4.5, 2.2 Hz, 1H), 2.05-1.86 (m, 2H), 1.05-0.95 (m, 2H), 0.95-0.87 (m, 2H). LC/MS (ESI⁺) m/z=270.8 [M+H]⁺.

### Method Int-10a

### Intermediate 12: 4-((2R,4S,6R)-4-bromo-6-methyltetrahydro-2H-pyran-2-yl)-1-cyclopropyl-1H-pyrazole

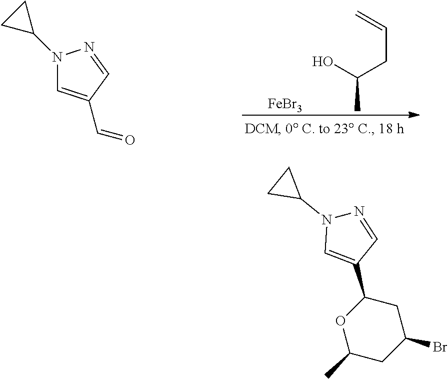

To iron (iii) bromide (3.20 g, 10.8 mmol) in a flame-dried 40 mL pressure vial equipped with a stir bar under argon was added a solution of 1-cyclopropylpyrazole-4-carbaldehyde (1.23 g, 9.03 mmol) and (2R)-pent-4-en-2-ol (778 mg, 9.03 mmol) in DCM (17 mL) under N₂ at 0° C. The reaction mixture was warmed to r.t. and stirred overnight. Water was added (20 mL), and the mixture was stirred for 30 mins. The product was extracted with DCM, and the combined organic layers were washed with brine, dried over Na₂SO₄, filtered, and concentrated in vacuo. The crude material was purified by silica gel chromatography eluting with 0-30% EtOAc in hexanes, followed by reverse phase chromatography eluting with 5-95% MeCN in H₂O to provide 4-[(2R,4S,6R)-4-bromo-6-methyl-tetrahydropyran-2-yl]-1-cyclopropyl-pyrazole (612 mg, 2.10 mmol, 23% yield) as a clear syrup. ¹H NMR (400 MHz, Chloroform-d) δ ppm 7.66-7.34 (m, 2H), 4.36 (dd, J=11.4, 2.0 Hz, 1H), 4.22 (tt, J 12.1, 4.5 Hz, 1H), 3.60 (ddd, J=11.0, 6.2, 1.9 Hz, 1H), 3.54 (tt, J 7.3, 3.9 Hz, 1H), 2.45 (ddt, J 13.0, 4.4, 2.0 Hz, 1H), 2.28 (ddt, J 12.9, 4.1, 2.0 Hz, 1H), 2.06 (q, J 12.0 Hz, 1H), 1.78 (td, J=12.5, 11.0 Hz, 1H), 1.25 (d, J=6.2 Hz, 3H), 1.13-1.05 (m, 2H), 1.04-0.94 (m, 2H). LC/MS (ESI⁺) m/z=285.0 [M+H]⁺.

### Method Int-10b

### Intermediate 52: 4-((2R,6R)-4-iodo-6-methyltetrahydro-2H-pyran-2-yl)-1-cyclopropyl-1H-pyrazole

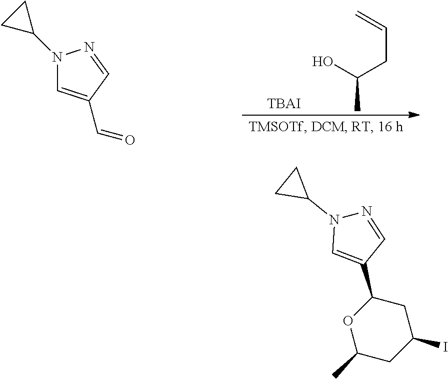

To a solution of 2-cyclopropyl-4H-imidazole-4-carbaldehyde (1.00 eq, 2000 mg, 14.7 mmol), (2R)-pent-4-en-2-ol (1.19 eq, 1500 mg, 17.4 mmol) and tetrabutylammonium iodide (1.20 eq, 6500 mg, 17.6 mmol) was added trimethylsilyl trifluoromethanesulfonate (1.19 eq, 3.2 ml, 17.5 mmol) dropwise. The mixture was stirred at 25° C. for 16 h. The mixture was concentrated under reduced pressure, and the residue was quenched with saturated Na₂S₂O₃ solution and extracted with EtOAc (30 mL*3). The combined organic phases were washed with water and brine, dried over Na₂SO₄, filtered, concentrated and purified by reversed-phase chromatography (45% MeCN in water, 0.1% Formic acid) to give 1-cyclopropyl-4-[(2R,6R)-4-iodo-6-methyl-tetrahydropyran-2-yl]pyrazole (1350 mg, 4.06 mmol, 27.67% yield) as a yellow oil. LCMS: (M+H)⁺=333.0; 100% purity (UV 254 nm); Retention time=1.88 min.

TABLE 3
Intermediate 59 was prepared following the procedure described in Method Int-10b, as
follows:

| Int # | Structure | Name | Starting Material 1 | Starting Material 2 |
|---|---|---|---|---|
| 59 | 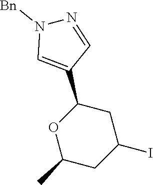 [SMILES: C[C@@H]1CC(I)C[C@H](c2cnn(Cc3ccccc3)c2)O1] | 1-benzyl-4- ((2R,6R)-4-iodo-6- methyltetrahydro- 2H-pyran-2-yl)-1H- pyrazole | 1-benzyl-1H- pyrazole-4- carbaldehyde | (2R)-pent-4-en-2-ol |

### Method Int-11

### Intermediate 13: 4-((2R,4S)-4-bromotetrahydro-2H-pyran-2-yl)-1-methyl-1H-pyrazole

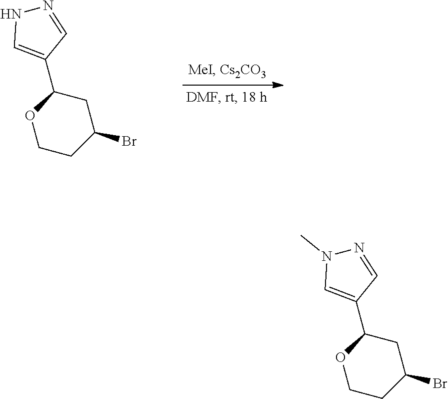

To a solution of 4-((2R,4S)-4-bromotetrahydro-2H-pyran-2-yl)-1H-pyrazole (25 mg, 0.108 mmol) in DMF (2.2 mL) was added cesium carbonate (88 mg, 0.270 mmol), followed by methyl iodide (0.0081 mL, 0.130 mmol). The reaction was stirred at r.t. overnight. Water was added, and the product was extracted with EtOAc. The combined organic layers were washed several times with H₂O, then brine, dried over Na₂SO₄, filtered, and concentrated in vacuo. The crude material was purified by silica gel chromatography eluting with 0-5% MeOH in DCM to provide 4-((2R,4S)-4-bromotetrahydro-2H-pyran-2-yl)-1-methyl-1H-pyrazole (18 mg, 0.0734 mmol, 68% yield) as a colorless solid. ¹H NMR (400 MHz, DMSO-d₆) δ ppm 7.64 (s, 1H), 7.36 (s, 1H), 4.50 (tt, J 12.0, 4.6 Hz, 1H), 4.33 (d, J=11.3 Hz, 1H), 3.90 (dd, J=11.8, 4.8 Hz, 1H), 3.78 (s, 3H), 3.50 (td, J=11.8, 2.0 Hz, 1H), 2.41 (d, J=12.5 Hz, 1H), 2.17 (dd, J=9.9, 6.4 Hz, 1H), 2.04-1.85 (m, 2H). LC/MS (ESI⁺) m/z=245.0 [M+H]⁺. The absolute configuration of the starting material 4-((2R,4S)-4-bromotetrahydro-2H-pyran-2-yl)-1H-pyrazole was elucidated by X-ray crystallography.

### Method Int-12

### Intermediate 14: 2-(2-methylpyridin-4-yl)morpholine

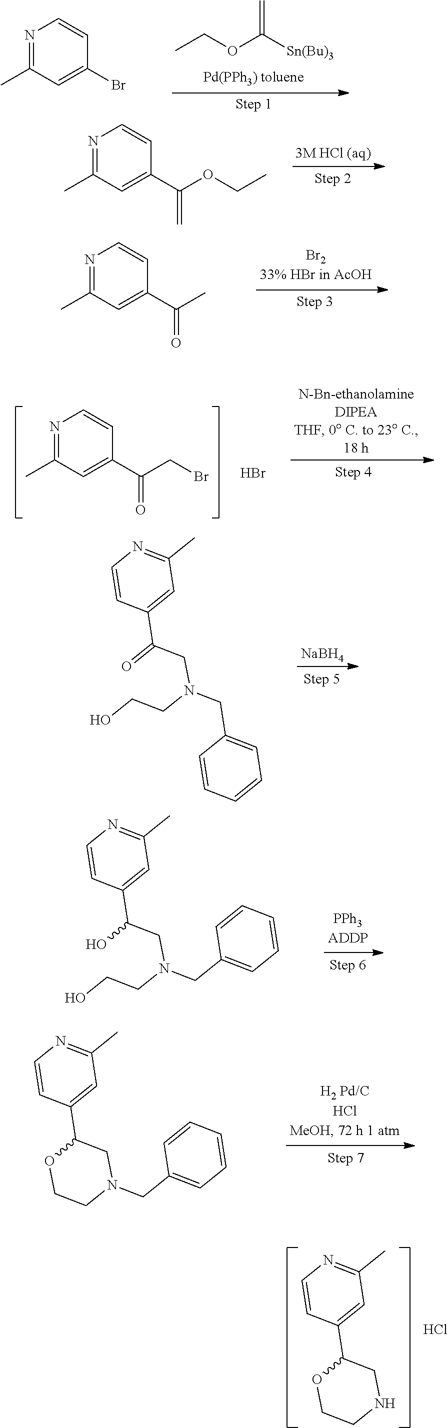

Step 1: A 250 mL pressure vessel was charged with 4-bromo-2-methylpyridine (6.90 mL, 58.1 mmol), 1-ethoxyvinyltributyltin (21.6 mL, 63.9 mmol, 1.1 equiv.) and toluene (100 mL) was purged N₂ gas at rt for 10 min. Tetrakis(triphenylphosphine)palladium (2.04 g, 2.91 mmol, 5 mol %) was added under N₂ atmosphere and the reaction mixture was purged with N₂ gas for 5 min at rt. The reaction vessel was sealed and stirred at 110° C. for 16 h. When the reaction was judged complete by LCMS, the reaction mixture was cooled to rt and KF (3.72 g, 1.1 equiv.), Na₂CO₃ (6.78 g, 1.1 equiv.) and silica (30 g) were added. The reaction mixture was stirred for 10 min and filtered through a pad of celite. The celite bed was washed with hexane (50 mL) and the combined filtrate was concentrated under reduced pressure. The crude residue was purified by column chromatography using silica gel, eluting with 0-5% EtOAc in hexane to afford 4-(1-ethoxyvinyl)-2-methylpyridine as a colorless oil (7.46 g, 79%). ¹H NMR (400 MHz, DMSO-d6): δ_{H} 8.41 (d, J=5.2 Hz, 1H), 7.35 (s, 1H), 8.41 (d, J=4.7 Hz, 1H), 5.01 (s, 1H), 4.46 (s, 1H), 3.91 (q, J 6.9 Hz, 2H), 2.47 (s, 3H), 1.35 (t, J 6.9 Hz, 3H). ESI-MS (m/z+): 164.2 [M+H]⁺, LC-RT: 0.505 min.

Step 2: A suspension of 5-(1-ethoxyvinyl)-2-methylpyridine (7.46 g, 45.7 mmol) in 3M HCl (30.5 mL, 91.4 mmol, 2 equiv.) was stirred at rt for 30 min. When the reaction was judged to be complete by LCMS, the reaction mixture was diluted with water (60 mL), basified to pH 11 with 5M NaOH and extracted with EtOAc (3×60 mL). The organic layer was dried (Na₂SO₄), filtered and concentrated under reduced pressure to afford 1-(2-methylpyridin-4-yl)ethan-1-one as a colorless oil (5.35 g, 82%). ¹H NMR (400 MHz, DMSO-d6): ^{6H} 8.65 (d, J=5.0 Hz, 1H), 7.69 (s, 1H), 7.60 (d, J=4.2 Hz, 1H), 2.49 (s, 3H), 2.57 (s, 3H). ESI-MS (m/z+): 136.10 [M+H]⁺, LC-RT: 0.202 min.

Step 3: A 100 mL round bottom flask was charged with 1-(2-methylpyridin-4-yl)ethan-1-one (5.00 g, 37.0 mmol) and HBr (33% in AcOH, 21 mL). The reaction mixture was cooled to 0° C. using an ice/water bath and a solution of bromine (1.9 mL, 37.0 mmol, 1.0 equiv.) in HBr (33% in AcOH, 7 ml) was added dropwise. The reaction mixture was stirred at 40° C. for 1 h and then further stirred at 80° C. for 1 h. When the reaction was judged complete by LCMS, the reaction mixture was cooled to rt, poured in Et₂O (100 mL) and stirred at rt for 30 min. The precipitate was filtered, washed with Et₂O (50 mL) and dried under reduced pressure to afford 2-bromo-1-(2-methylpyridin-4-yl)ethan-1-one (HBr salt) as a yellow solid (10.7 g, 96%). ESI-MS (m/z+): 274.0 [M+H]⁺, LC-RT: 1.459 min.

Step 4: To a solution of 2-bromo-1-(2-methylpyridin-4-yl)ethan-1-one acetate (10.7 g, 39.0 mmol) in THF (182 mL) at 0° C. was slowly added N-benzylethanolamine (5.54 mL, 39.0 mmol, 1.0 equiv.) followed by DIPEA (13.6 mL, 78.1 mmol). The reaction was slowly warmed to r.t. overnight, after which a precipitate formed. The solvent was removed in vacuo. Water was then added to the reaction mixture and the aqueous phase was extracted with EtOAc (3×100 mL). The combined organic phases were dried over Na₂SO₄, filtered, and concentrated in vacuo to provide 2-(benzyl(2-hydroxyethyl)amino)-1-(2-methylpyridin-4-yl)ethan-1-one (11.1 g, 100%) as a yellow solid. ESI-MS (m/z+): 285.10 [M+H]⁺, LC-RT: 0.642 min.

Step 5: A 500 mL round bottom flask was charged with 2-(benzyl(2-hydroxyethyl)amino)-1-(2-methylpyridin-4-yl)ethan-1-one (11.10 g, 39.0 mmol, 1 equiv.) in methanol (390 mL) and was cooled to 0° C. Sodium borohydride (2.95 g, 78.1 mmol, 2.0 equiv.) was added portion wise then the reaction was gradually warmed to r.t. over 12 h. When the reaction was judged to be complete by LCMS, the solution was cooled to 0° C., and water (250 mL) was added. The product was extracted with EtOAc (3×100 mL), and the combined organic layers were washed with brine, dried over Na₂SO₄, filtered, and concentrated in vacuo to give the pure product 2-(benzyl(2-hydroxyethyl)amino)-1-(2-methylpyridin-4-yl)ethan-1-ol (8.45 g, 29.5 mmol, 75.6%) as a clear oil. ESI-MS (m/z+): 287.20 [M+H]⁺, LC-RT: 0.215 min.

Step 6: A flame-dried 50 mL round bottom flask under nitrogen was charged with 4-benzyl-2-(2-methyl-4-pyridyl)morpholine (1.00 eq, 1.35 g, 5.03 mmol), Pd/C (0.252 eq, 135 mg, 1.27 mmol) and HCl (4M in dioxanes, 1.00 eq, 5.03 mmol). The reaction vial was purged with N₂ then the reaction mixture was bubbled with H₂ for 2 min. The needle was removed from the solution and the reaction was stirred at r.t. under positive pressure of H₂ (balloon) overnight. Complete conversion was observed by TLC and LCMS. The reaction mixture was filtrated on a pad of Celite and the solvent was removed in vacuo to yield the desired 2-(2-methyl-4-pyridyl)morpholine hydrochloride (1.01 g, 4.70 mmol, 93.51%). ESI-MS (m/z+): 179.1 [M+H]⁺, LC-RT: 0.240 min. ¹H NMR (DMSO-d6, 400 MHz): δ_{H} 8.53 (1H, d, J=5.4 Hz), 7.45 (1H, s), 7.36 (1H, d, J=5.3 Hz), 4.94 (1H, d, J=11.0 Hz), 4.13 (1H, d, J=12.7 Hz), 4.00 (1H, t, J 12.3 Hz), 3.52 (1H, d, J=12.7 Hz), 3.06 (1H, t, J 12.4 Hz), 2.90 (1H, t, J 11.9 Hz), 2.54 (3H, s).

### Method Int-13

### Intermediate 15: 2-(1-cyclopropylpyrazol-4-yl)morpholin-4-ium chloride

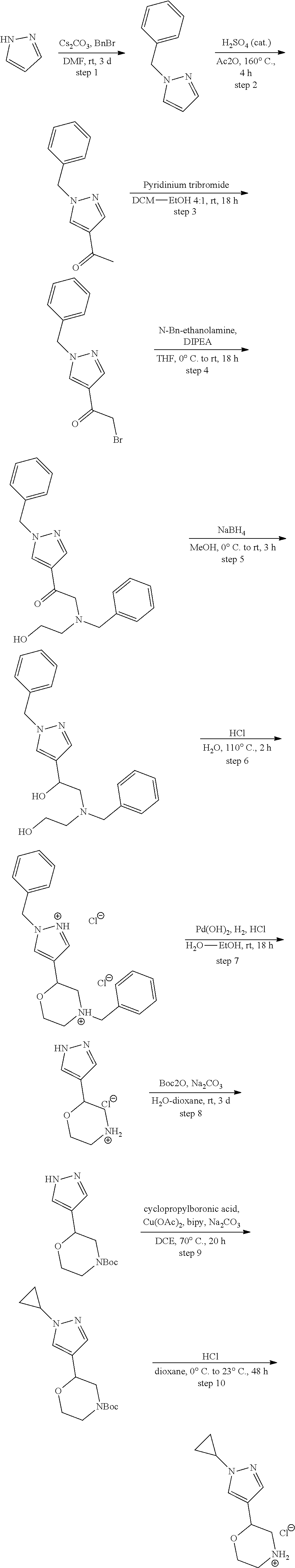

Step 1: To a solution of pyrazole (5.6 g, 81.9 mmol) in DMF (150 mL) at 0° C. was added cesium carbonate (48.5 g, 149 mmol), followed by benzyl bromide (9.2 mL, 74.5 mmol). The reaction stirred for 3 days at r.t. Water was added, and the product was extracted with EtOAc. The combined organic layers were washed several times with H₂O, then brine, dried over Na₂SO₄, filtered, and concentrated in vacuo to provide 1-benzyl-1H-pyrazole (11.8 g, 74.6 mmol, 94% yield) as a yellow liquid, which was taken to the next step without further purification. ¹H NMR (400 MHz, Chloroform-d) δ ppm 7.56 (d, J=1.9 Hz, 1H), 7.41-7.27 (m, 4H), 7.24-7.18 (m, 2H), 6.28 (t, J 2.2 Hz, 1H), 5.33 (s, 2H). LC/MS (ESI⁺) m/z=159.0 [M+H]⁺.

Step 2: To a solution of 1-benzyl-1H-pyrazole (5.1 g, 32.3 mmol) in acetic anhydride (11.0 mL, 116 mmol) was added sulfuric acid (0.17 mL, 3.23 mmol). The solution was refluxed for 4 hours. The reaction was cooled to r.t., and water was added. The mixture was cooled to 0° C. and basified with NaOH to pH >10. The product was extracted with DCM, and the combined organic layers were washed with brine, dried over Na₂SO₄, filtered, and concentrated in vacuo. The crude material was purified by silica gel chromatography eluting with 10-40% EtOAc in hexanes to provide 1-(1-benzyl-1H-pyrazol-4-yl)ethan-1-one-1 (4.21 g, 21.0 mmol, 64% yield) as a beige solid. ¹H NMR (400 MHz, Chloroform-d) δ ppm 7.93 (s, 1H), 7.84 (s, 1H), 7.46-7.31 (m, 3H), 7.29-7.24 (m, 2H), 5.31 (s, 2H), 2.41 (s, 3H). LC/MS (ESI⁺) m/z=201.1 [M+H]⁺.

Step 3: To a solution of 1-(1-benzyl-1H-pyrazol-4-yl)ethan-1-one (10.6 g, 52.9 mmol) in DCM (85 mL) and EtOH (21.2 mL) was added pyridinium tribromide (18.8 g, 52.9 mmol). The reaction stirred overnight at r.t. The reaction was diluted with water (50 mL), and sodium sulfite (1.7 g, 13.2 mmol) was added. The mixture stirred for 20 minutes. The layers were separated, and the product was extracted with DCM. The combined organic layers were washed with brine, dried over Na₂SO₄, filtered, and concentrated in vacuo. The crude material was purified by silica gel chromatography eluting with 0-30% EtOAc in hexanes to provide 1-(1-benzyl-1H-pyrazol-4-yl)-2-bromoethan-1-one (11.5 g, 41.2 mmol, 77% yield) as a white solid. ¹H NMR (400 MHz, Chloroform-d) δ ppm 8.01 (s, 1H), 7.94 (s, 1H), 7.47-7.33 (m, 3H), 7.33-7.21 (m, 2H), 5.33 (s, 2H), 4.16 (d, J=1.4 Hz, 2H). LC/MS (ESI⁺) m/z=279.0 [M+H]⁺.

Step 4: To a solution of 1-(1-benzyl-1H-pyrazol-4-yl)-2-bromoethan-1-one-1 (6.0 g, 21.5 mmol) in THF (100 mL) at 0° C. was slowly added N-benzylethanolamine (3.1 mL, 21.5 mmol) and N,N-diisopropylethylamine (7.5 mL, 43.0 mmol). The reaction was slowly warmed to r.t. overnight. The solvent was removed in vacuo. Water was then added to the reaction mixture, and the product was extracted with EtOAc. The combined organic layers were dried over Na₂SO₄, filtered, and concentrated in vacuo. The crude material was purified by silica gel chromatography eluting with 0-10% MeOH in DCM to provide 2-(benzyl(2-hydroxyethyl)amino)-1-(1-benzyl-1H-pyrazol-4-yl)ethan-1-one (7.0 g, 20.1 mmol, 91% yield) as a yellow semi-solid. ¹H NMR (400 MHz, DMSO-d₆) δ ppm 8.57 (s, 1H), 7.96 (s, 1H), 7.20-7.31 (m, 10H), 5.36 (s, 2H), 4.44 (t, J 5.2 Hz, 1H), 3.68 (d, J=3.1 Hz, 2H), 3.43-3.53 (m, 4H), 2.60 (d, J=6.2 Hz, 2H). LC/MS (ESI⁺) m/z=349.9 [M+H]⁺.

Step 5: To a solution of 2-[benzyl(2-hydroxyethyl)amino]-1-(1-benzyl-1H-pyrazol-4-yl)ethanone (6.7 g, 19.9 mmol) in methanol (133 mL) at 0° C. was added sodium borohydride (1.5 g, 39.8 mmol) very slowly. The reaction mixture was stirred at 0° C. for 30 min and then at r.t. for 3 hours. The solvent was removed in vacuo (˜90%), and the mixture was cooled to 0° C. Water was added slowly, and the product was extracted with EtOAc. The combined organic layers were washed with brine, dried over Na₂SO₄, filtered, and concentrated in vacuo to provide 2-[benzyl(2-hydroxyethyl)amino]-1-(1-benzyl-1H-pyrazol-4-yl)ethan-1-ol (6.6 g, 17.9 mmol, 90% yield) as a yellow semi-solid, which was taken to the next step without purification. ¹H NMR (400 MHz, DMSO-d6) δ ppm 7.62 (s, 1H), 7.45-7.12 (m, 10H), 5.25 (s, 2H), 4.81 (d, J=3.8 Hz, 1H), 4.70-4.54 (m, 1H), 4.37 (t, J 5.6 Hz, 1H), 3.82-3.60 (m, 2H), 3.42 (p, J 5.8 Hz, 2H), 2.73-2.52 (m, 3H). LC/MS (ESI⁺) m/z=352.2 [M+H]⁺.

Step 6: A solution of 2-[benzyl(2-hydroxyethyl)amino]-1-(1-benzyl-1H-pyrazol-4-yl)ethan-1-ol (6.4 g, 18.2 mmol) in 6M aqueous HCl (46 mL, 277 mmol) was refluxed at 110° C. for 2 hours. The solution was concentrated in vacuo and dried under high vacuum to provide 4-benzyl-2-(1-benzyl-1H-pyrazol-2-ium-4-yl)morpholin-4-ium dichloride (7.65 g, 18.8 mmol, quantitative yield) as a beige foam, which was taken to the next step without purification. ¹H NMR (400 MHz, DMSO-d₆) δ ppm 12.17 (s, 1H), 7.87 (s, 1H), 7.71-7.64 (m, 2H), 7.60 (s, 1H), 7.48 (s, 1H), 7.46-7.40 (m, 3H), 7.37-7.24 (m, 3H), 7.24-7.15 (m, 2H), 5.29 (s, 2H), 5.00 (dd, J=11.1, 2.3 Hz, 1H), 4.58-4.22 (m, 2H), 4.16-3.90 (m, 2H), 3.36 (d, J=12.1 Hz, 1H), 3.27-2.98 (m, 3H). LC/MS (ESI⁺) m/z=334.2 [M+H]⁺.

Step 7: To a solution of 4-benzyl-2-(1-benzyl-1H-pyrazol-2-ium-4-yl)morpholin-4-ium dichloride (2.00 g, 4.92 mmol) in ethanol (12 mL) and water (12 mL) was added 2M aqueous HCl (7.4 mL, 14.8 mmol). The solution was purged with argon via balloon and outlet for 5 minutes. Palladium hydroxide on carbon (276 mg, 0.98 mmol) was added quickly, and the mixture was purged with argon via balloon and outlet again for 5 minutes. The argon balloon was replaced with a hydrogen balloon, and the reaction stirred at r.t. overnight. The mixture was filtered over celite and washed with ethanol and water several times. The filtrate was concentrated in vacuo to provide 2-(1H-pyrazol-4-yl)morpholin-4-ium chloride (1.13 g, 4.91 mmol, quantitative yield) as a white solid, which was lyophilized and taken to the next step without purification. ¹H NMR (400 MHz, DMSO-d₆) δ ppm 13.03 (s, 1H), 10.03 (s, 2H), 7.76 (s, 1H), 7.53 (s, 1H), 4.83 (d, J=10.9 Hz, 1H), 3.95 (d, J=7.8 Hz, 2H), 3.25 (d, J=12.5 Hz, 1H), 3.11 (d, J=12.7 Hz, 1H), 2.97 (q, J 11.5, 10.7 Hz, 2H). LC/MS m/z=(ESI⁺) 154.1 [M+H]⁺.

Step 8: To a solution of 2-(1H-pyrazol-4-yl)morpholin-4-ium chloride (1.5 g, 7.91 mmol) in water (100 mL) and 1,4-Dioxane (50 mL) was added sodium carbonate (2.5 g, 23.7 mmol), followed by di-tert-butyl dicarbonate (2.1 g, 9.49 mmol), and the reaction stirred at r.t. for 3 days. The mixture was concentrated in vacuo to dryness and purified directly by silica gel chromatography eluting with 30-100% EtOAc in hexanes to provide tert-butyl 2-(1H-pyrazol-4-yl)morpholine-4-carboxylate (705 mg, 2.78 mmol, 35% yield) as a white solid. ¹H NMR (400 MHz, Chloroform-d) δ ppm 10.52-10.15 (m, 1H), 7.60 (s, 2H), 4.60-4.21 (m, 1H), 4.10-3.99 (m, 1H), 3.98-3.75 (m, 2H), 3.65 (td, J=11.4, 2.8 Hz, 1H), 3.02 (d, J=35.1 Hz, 2H), 1.48 (s, 9H). LC/MS (ESI⁺) m/z=254.2 [M+H]⁺.

Step 9: To a solution of tert-butyl 2-(1H-pyrazol-4-yl)morpholine-4-carboxylate (1.07 g, 4.25 mmol) in dichloroethane (28 mL) was added cyclopropylboronic acid (730 mg, 8.50 mmol) and sodium carbonate (1.35 g, 12.8 mmol). The reaction mixture was heated to 70° C. A solid mixture of copper(II) acetate (781 mg, 4.25 mmol) and 2,2′-dipyridyl (664 mg, 4.25 mmol) was added to the reaction mixture in one portion. The reaction stirred under oxygen atmosphere at 70° C. overnight. The mixture was cooled to r.t. and concentrated in vacuo. To the residue was added saturated NaHCO₃, and the product was extracted with EtOAc. The combined organic layers were washed with brine, dried over Na₂SO₄, filtered, and concentrated in vacuo. The crude material was purified by silica gel chromatography eluting with 0-60% EtOAc in hexanes to provide tert-butyl 2-(1-cyclopropylpyrazol-4-yl)morpholine-4-carboxylate (0.97 g, 3.30 mmol, 78% yield) as a yellow oil. ¹H NMR (400 MHz, Chloroform-d) δ ppm 7.45 (s, 2H), 4.41 (d, J=10.2 Hz, 1H), 4.11-3.71 (m, 3H), 3.68-3.50 (m, 2H), 3.15-2.84 (m, 2H), 1.47 (s, 9H), 1.15-1.04 (m, 2H), 1.02-0.94 (m, 2H). LC/MS (ESI⁺) m/z=294.1 [M+H]⁺.

Step 10: To a solution of tert-butyl 2-(1-cyclopropylpyrazol-4-yl)morpholine-4-carboxylate (1.14 g, 3.88 mmol) in 1,4-dioxane (19 mL) at 0° C. was added HCl (4M in 1,4-dioxane) (8.0 mL, 77.6 mmol) dropwise. The solution was warmed to r.t. and stirred for 2 days. The solution was concentrated in vacuo to dryness to provide 2-(1-cyclopropylpyrazol-4-yl)morpholin-4-ium chloride (894 mg, 3.83 mmol, 99% yield) as a beige solid, which was used in the next step without purification. ¹H NMR (400 MHz, DMSO-d₆) δ ppm 9.96-9.42 (m, 2H), 7.86 (s, 1H), 7.45 (s, 1H), 4.73 (dd, J=11.4, 2.8 Hz, 1H), 3.98 (dd, J=12.7, 3.8 Hz, 1H), 3.88 (dd, J=13.8, 11.1 Hz, 1H), 3.74-3.59 (m, 1H), 3.32 (d, J=12.4 Hz, 1H), 3.19 (d, J=12.6 Hz, 1H), 3.10-2.92 (m, 2H), 1.04-0.96 (m, 2H), 0.97-0.89 (m, 2H). LC/MS (ESI⁺) m/z=194.1 [M+H]⁺.

### Method Int-14

### Intermediate 16: 2-(1-cyclopropyl-1H-pyrazol-4-yl)-6-methylmorpholine

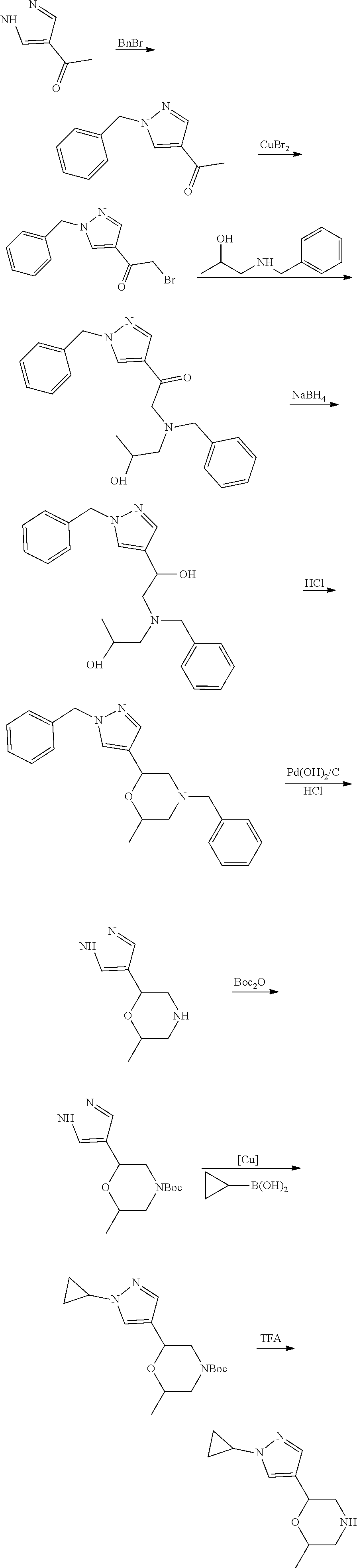

Step 1: To a stirred solution of 1-(1H-pyrazol-4-yl) ethan-1-one (10 g, 0.1 mol) and Cs₂CO₃ (48.3 g, 0.15 mol) in DMF (100 mL) was added (bromomethyl)benzene (20.3 g, 0.12 mol) drop wise at room temperature under N₂. The reaction was stirred at 80° C. for 1 h. The mixture was poured into water (500 mL) and extracted with EA (100 mL×3). The organic phase was washed with brine (100 mL×2), dried over Na₂SO₄ and filtered. The filtration was concentrated under vacuum, the residue was purified by column chromatography on silica gel (PE:EA=5:1) to afford 1-(1-benzyl-1H-pyrazol-4-yl) ethan-1-one (16.0 g) as a light yellow solid. LCMS: (M+H)+=201.1; purity=97.36% (UV 254 nm).

Step 2: To a solution of 1-(1-benzyl-1H-pyrazol-4-yl)ethan-1-one (3.9 g, 19.47 mmol) in 1,4-dioxane (40 mL) was added CuBr₂ (7.23 g, 32.37 mmol) at rt. After addition, the reaction mixture was stirred at 85° C. for 7 h. The reaction mixture was poured into water (160 mL) and extracted with EA (80 mL×3). The combined organic layers were dried over anhydrous Na₂SO₄, filtered and concentrated. The crude product was purified by silica gel column (PE/EA, 1:10 to 1:5) to give 1-(1-benzyl-1H-pyrazol-4-yl)-2-bromoethan-1-one (2.9 g, 10.39 mmol) as a white solid. LCMS: (M+H)+=280.

Step 3: To a solution of compound 1-(1-benzyl-1H-pyrazol-4-yl)-2-bromoethan-1-one (2.9 g, 10.39 mmol) in THF (20 mL) at room temperature was slowly added 1-(benzylamino)propan-2-ol (1.89 g, 11.44 mmol) under N₂. The reaction mixture was stirred at 35° C. for 3 hour to give a yellow solution. Water (20 mL) was added drop wise to quench the reaction. The reaction mixture was extracted with EA (50 mL×3). The combined organic layer was dried over anhydrous Na₂SO₄, filtered and concentrated under reduced pressure. The combined crude material was absorbed onto a plug of silica gel and purified by chromatography through a silica gel column eluting with a silica gel column (PE/EA, 1:10 to 1:2) provide compound 2-(benzyl(2-hydroxypropyl)amino)-1-(1-benzyl-1H-pyrazol-4-yl)ethan-1-one (2.81 g, 7.73 mmol). LCMS: (M+H)⁺=364.

Step 4: To a solution of compound 2-(benzyl(2-hydroxypropyl)amino)-1-(1-benzyl-1H-pyrazol-4-yl)ethan-1-one (2.8 g, 7.70 mmol) in methanol (28 mL) at 0° C. was added sodium tetrahydroborate (0.58 g, 15.40 mmol) portion wise. The reaction mixture was stirred at 0° C. for 30 min and then at room temperature for 2 h. Ice-cooled water (20 mL) was added drop wise to quench the reaction. The reaction mixture was extracted with EA (50 mL×3). The combined organic layers were dried over anhydrous Na₂SO₄, filtered and concentrated to give 1-(benzyl(2-(1-benzyl-1H-pyrazol-4-yl)-2-hydroxyethyl)amino)propan-2-ol (2.8 g, 7.66 mmol) as a yellow liquid compound, which was used directly for next step without further purification. LCMS: (M+H)⁺=366.

Step 5: To a solution of compound 1-(benzyl(2-(1-benzyl-1H-pyrazol-4-yl)-2-hydroxyethyl)amino)propan-2-ol (2.8 g, 7.66 mmol) in 1,4-dioxane (15 mL) at room temperature was slowly added 6M HCl (15 ml). The reaction mixture was stirred at 110° C. for 4 h. 15% KOH was added drop wise to quench the reaction, adjust pH 8-9. The reaction mixture was extracted with EA (100 mL×3). The combined organic layers were dried over anhydrous Na₂SO₄, filtered and concentrated to give 4-benzyl-2-(1-benzyl-1H-pyrazol-4-yl)-6-methylmorpholine (2.39 g, 6.88 mmol) as a yellow liquid compound, which was used directly for next step without further purification. LCMS: (M+H)⁺=348.

Step 6: To a solution of 4-benzyl-2-(1-benzyl-1H-pyrazol-4-yl)-6-methylmorpholine (2.39 g, 6.88 mmol) in methanol (12 mL) and 2.4 mL HCl (6 M) was added Pd(OH)₂C (0.48 g), the reaction mixture was stirred at 30° C. for 16 h. The reaction mixture was filtered and the filtrate was concentrated under vacuum, the residue was adjusted ph to 9-10 by Na₂CO₃ aq. The aqueous phase was directly used in next step. LCMS: (M+H)⁺=168.

Step 7: To a solution of step 6 in water/1,4-dioxane (10 mL/0 mL) was added Na₂CO₃ (0.88 g, 8.30 mml) and BoC₂O (1.58 g, 7.24 mmol). The reaction mixture was stirred at room temperature for 1 h. The reaction mixture was poured into water (20 mL) and extracted with EA (50 mL×3). The combined organic layers were dried over anhydrous Na₂SO₄, filtered and concentrated to give tert-butyl 2-methyl-6-(1H-pyrazol-4-yl)morpholine-4-carboxylate crude. The crude product was directly used in next step. LCMS: (M+H)⁺=268.

Step 8: To a solution of tert-butyl 2-methyl-6-(1H-pyrazol-4-yl)morpholine-4-carboxylate (1.77 g, 6.62 mmol) in DMF (35 mL) was added to cyclopropylboronic acid (1.71 g, 19.9 mmol), Cu(OAc)₂ (1.32 g, 7.27 mmol), Na₂CO₃ (1.40 g, 13.2 mmol), 2,2′-Dipyridyl(1.14 g, 7.30 mmol) at room temperature. The reaction mixture was stirred at 80° C. for 10 h. The mixture was poured into water (100 mL) and extracted with EA (60 mL×3). The organic phase was washed with brine (60 mL×2), dried over Na₂SO₄ and filtered. The filtrate was concentrated under vacuum, The crude product was purified by silica gel column (PE/EA, 1:10 to 1:5) to give tert-butyl 2-(1-cyclopropyl-1H-pyrazol-4-yl)-6-methylmorpholine-4-carboxylate (1.6 g, 5.20 mmol) as a yellow liquid. LCMS: (M+H)⁺=308.

Step 9: To a solution of tert-butyl 2-(1-cyclopropyl-1H-pyrazol-4-yl)-6-methylmorpholine-4-carboxylate (1.6 g, 5.20 mmol) in dichloromethane (10 mL) was added TFA (3 mL), The reaction mixture was stirred at room temperature for 1 h. The filtrate was concentrated under vacuum to give 2-(1-cyclopropyl-1H-pyrazol-4-yl)-6-methylmorpholine (1.02 g, 4.93 mmol) as a yellow liquid. LCMS: (M+H)⁺=208.

### Method Int-15

### Intermediate 17: 7-chloro-2-methyl-5-(3-(trifluoromethyl)bicyclo[1.1.1]pentan-1-yl)pyrido[3,4-b]pyrazine

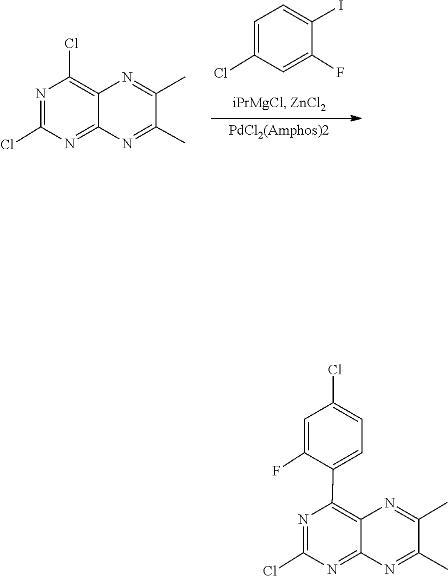

To a 100 mL round-bottomed as was added 4-chloro-2-fluoro-1-iodobenzene (1.0 g, 3.91 mmol) in THF (10 mL). The mixture was cooled to −40° C. and isopropylmagnesium chloride (2.144 mL, 10.73 mmol) was added dropwise at −40° C. and stirred for 30 min. The reaction mixture was then cooled to −78° C. ZnCl₂ (2.05 mL, 3.9 mmol) (2 M solution in THF) was added drop wise and the reaction mixture was allowed to warm r.t, after which 20 mL of THF was added and stirred for 10 min, then the stirring was turned off in order to let the precipitates settle. The reaction mixture was directly used for the next step.

To a dry 100 mL round-bottomed flask was added bis(di-tert-butyl(4-dimethylaminophenyl)phosphine)-dichloropalladium (46.4 mg, 0.065 mmol) and 2,4-dichloro-6,7-dimethylpteridine (0.3 g, 1.31 mmol). The mixture was purged with N₂, and dissolved in THF (3 mL). (4-chloro-2-fluorophenyl)zinc(II) iodide (11.9 mL, 1.31 mmol, as made by the procedure above) was added portion wise to the mixture at r.t. and stirred for 20 min. The reaction was quenched with sat. NaHCO₃ solution (20 mL). The aqueous layer was extracted with ethyl acetate (2×30 mL), dried over anhydrous Na₂SO₄ and concentrated to yield a residue. The residue was purified using an automated silica column (100) with 0-50% ethyl acetate in hexanes (product eluted at 40% ethyl acetate) to obtain 2-chloro-4-(4-chloro-2-fluorophenyl)-6,7-dimethylpteridine (1.0 g, 3.12 mmol, 71% yield) as a purple solid. LCMS: (M+H)⁺=323.0; purity=90.67% (214 nm).

TABLE 4
Intermediates 18 to 28 were prepared following the procedure described in Method Int-15:

| Int # | Structure | Name |
|---|---|---|
| 18 | 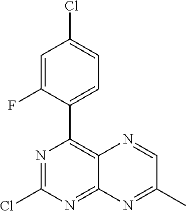 [SMILES: Cc1cnc2c(-c3ccc(Cl)cc3F)nc(Cl)nc2n1] | 2-chloro-4-(4-chloro-2-fluorophenyl)-7-methylpteridine |
| 19 | 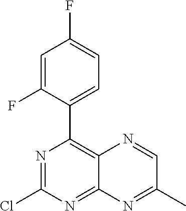 [SMILES: Cc1cnc2c(-c3ccc(F)cc3F)nc(Cl)nc2n1] | 2-chloro-4-(2,4-difluorophenyl)-7-methylpteridine |
| 20 | 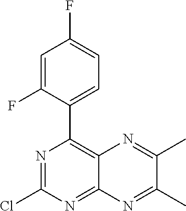 [SMILES: Cc1nc2nc(Cl)nc(-c3ccc(F)cc3F)c2nc1C] | 2-chloro-4-(2,4-difluorophenyl)-6,7-dimethylpteridine |
| 21 | 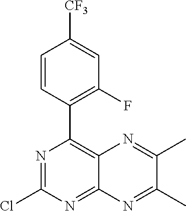 [SMILES: Cc1nc2nc(Cl)nc(-c3ccc(C(F)(F)F)cc3F)c2nc1C] | 2-chloro-4-(2-fluoro-4-(trifluoromethyl)phenyl)-6,7- dimethylpteridine |
| 22 | 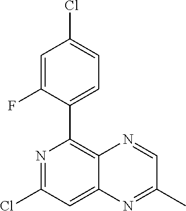 [SMILES: Cc1cnc2c(-c3ccc(Cl)cc3F)nc(Cl)cc2n1] | 7-chloro-5-(4-chloro-2-fluorophenyl)-2- methylpyrido[3,4-b]pyrazine |
| 23 | 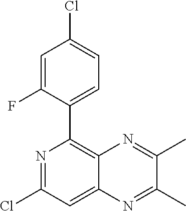 [SMILES: Cc1nc2cc(Cl)nc(-c3ccc(Cl)cc3F)c2nc1C] | 7-chloro-5-(4-chloro-2-fluorophenyl)-2,3- dimethylpyrido[3,4-b]pyrazine |
| 24 | 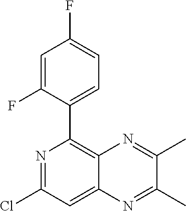 [SMILES: Cc1nc2cc(Cl)nc(-c3ccc(F)cc3F)c2nc1C] | 7-chloro-5-(2,4-difluorophenyl)-2,3-dimethylpyrido[3,4- b]pyrazine |
| 25 | 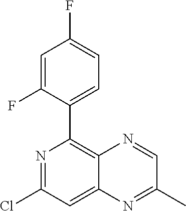 [SMILES: Cc1cnc2c(-c3ccc(F)cc3F)nc(Cl)cc2n1] | 7-chloro-5-(2,4-difluorophenyl)-2-methylpyrido[3,4- b]pyrazine |
| 26 | 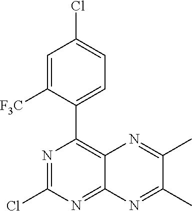 [SMILES: Cc1nc2nc(Cl)nc(-c3ccc(Cl)cc3C(F)(F)F)c2nc1C] | 2-chloro-4-[4-chloro-2-(trifluoromethyl)phenyl]-6,7- dimethyl-pteridine |
| 27 | 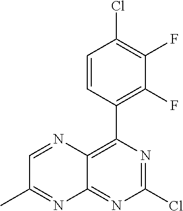 [SMILES: Cc1cnc2c(-c3ccc(Cl)c(F)c3F)nc(Cl)nc2n1] | 2-chloro-4-(4-chloro-2,3-difluoro-phenyl)-7-methyl- pteridine |
| 28 | 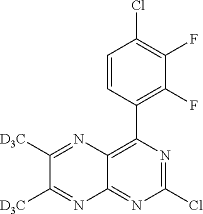 [SMILES: [2H]C([2H])([2H])c1nc2nc(Cl)nc(-c3ccc(Cl)c(F)c3F)c2nc1C([2H])([2H])[2H]] | 2-chloro-4-(4-chloro-2,3-difluoro-phenyl)-6,7- bis(trideuteriomethyl)pteridine |
| 59 | 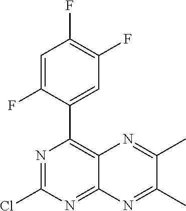 [SMILES: Cc1nc2nc(Cl)nc(-c3cc(F)c(F)cc3F)c2nc1C] | 2-chloro-6,7-dimethyl-4-(2,4,5-trifluorophenyl)pteridine |

### Method Int-16

### Intermediate 29: 2-chloro-4-(4-chloro-2,3-difluoro-phenyl)-6,7-dimethyl-pteridine

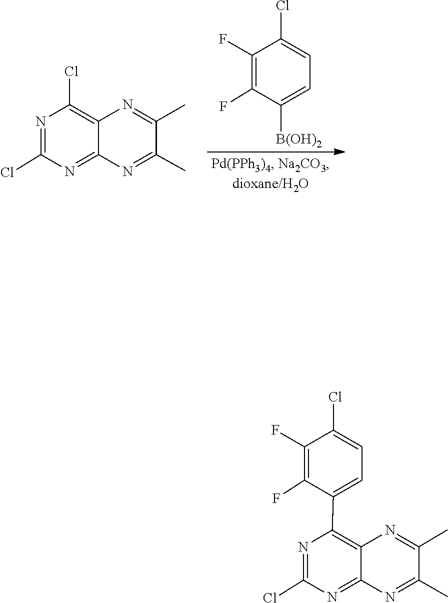

To a 20 mL microwave vial was added 2,4-dichloro-6,7-dimethyl-pteridine (500 mg, 2.18 mmol), (4-chloro-2,3-difluoro-phenyl)boronic acid (420 mg, 2.18 mmol), sodium carbonate (694 mg, 6.55 mmol), 1,4-dioxane (10 mL) and water (3 mL). The reaction mixture was degassed with nitrogen for 10 min. Pd(PPh₃)₄ (126 mg, 0.109 mmol) was added and the reaction mixture was heated at 40° C. for 3.5 h. The mixture was cooled to r.t., diluted with DCM (50 mL) and water (10 mL). The aqueous layer was extracted with DCM (2×20 mL). Combined organic layers were washed with brine (10 mL), dried over Na₂SO₄, and concentrated in vacuo. The crude residue was purified by silica gel chromatography (40 g SilicaSep column) using EtOAc and hexanes (50-60%) to obtain 2-chloro-4-(4-chloro-2,3-difluoro-phenyl)-6,7-dimethyl-pteridine (176 mg, 0.516 mmol, 24%) as a brown solid. ESI-MS (m/z+): 342.0 [M+H]⁺, LC-RT: 3.579 min. ¹H NMR (400 MHz, CDCl₃) δ ppm 7.53-7.46 (m, 1H), 7.43-7.35 (m, 1H), 2.86 (s, 3H), 2.75 (s, 3H). ¹⁹F NMR (376 MHz, CDCl₃) δ ppm −130.92 (s), -137.18 (s).

TABLE 5
Intermediates 30, 42 and 43 were prepared following the procedure described in Method
Int-16 using the starting materials indicated:

| Int # | Structure | Name | Starting Material 1 | Starting Material 2 |
|---|---|---|---|---|
| 30 | 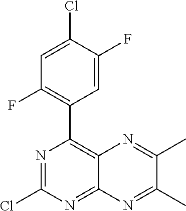 [SMILES: Cc1nc2nc(Cl)nc(-c3cc(F)c(Cl)cc3F)c2nc1C] | 2-chloro-4-(4-chloro- 2,5-difluoro-phenyl)- 6,7-dimethyl-pteridine | 2,4-dichloro-6,7- dimethyl- pteridine | (4-chloro-2,5- difluoro- phenyl)boronic acid |
| 42 | 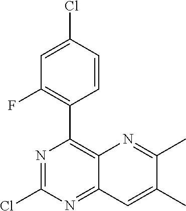 [SMILES: Cc1cc2nc(Cl)nc(-c3ccc(Cl)cc3F)c2nc1C] | 2-chloro-4-(4-chloro- 2-fluorophenyl)-6,7- dimethylpyrido[3,2- d]pyrimidine | 2,4-dichloro-6,7- dimethyl- pyrido[3,2- d]pyrimidine | (4-chloro-2- fluoro- phenyl)boronic acid |
| 43 | 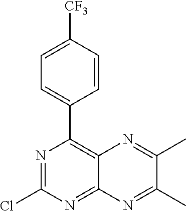 [SMILES: Cc1nc2nc(Cl)nc(-c3ccc(C(F)(F)F)cc3)c2nc1C] | 2-chloro-6,7-dimethyl- 4-(4- (trifluoromethyl)phenyl) pteridine | 2,4-dichloro-6,7- dimethyl- pteridine | (4- (trifluoromethyl) phenyl)boronic acid |

### Method Int-17

### Intermediate 31: 2-chloro-6,7-dimethyl-4-(6-(trifluoromethyl)pyridin-3-yl)pteridine

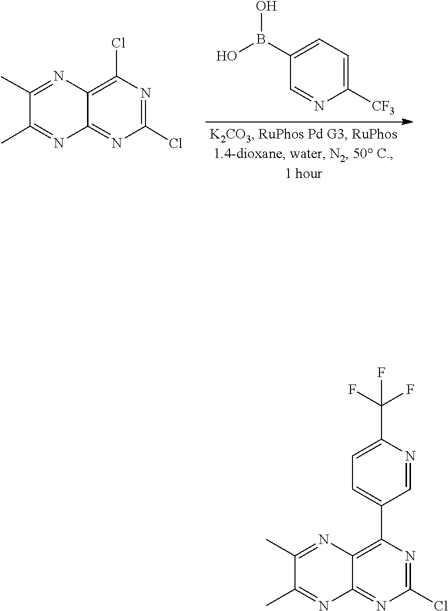 [SMILES: Cc1nc2nc(Cl)nc(Cl)c2nc1C ; OB(O)c1ccc(C(F)(F)F)nc1 ; Cc1nc2nc(Cl)nc(-c3ccc(C(F)(F)F)nc3)c2nc1C]

To a 20 mL sealed tube was added 2,4-dichloro-6,7-dimethylpteridine (2 eq, 1.2 g, 5.24 mmol) and 2-trifluoromethyl-pyridine-5-boronic acid (1 eq, 500 mg, 2.62 mmol), 1,4-dioxane (24.0 mL) and water (4.0 mL). Potassium carbonate (6 eq, 2.18 g, 15.8 mmol) was added and the reaction mixture was degassed with nitrogen for 10 min. RuPhos Pd G3 (0.1 eq, 200 mg, 283 μmol) was added and the reaction mixture was heated at 50° C. for 1 h. The mixture was cooled down to r.t., diluted with water (50.0 mL) and extracted with EtOAc (3×100 mL). The organic extracts were dried over Na₂SO₄, filtered and concentrated in vacuo. The crude material was purified by silica gel chromatography (120 g cartridge) using hexanes and EtOAc (50-60%) to afford 2-chloro-6,7-dimethyl-4-(6-(trifluoromethyl)pyridin-3-yl)pteridine as a brown solid (867 mg, 65% yield). ¹H NMR (400 MHz, CDCl₃) δ ppm 9.88 (s, 1H), 8.94 (d, J=8.3 Hz, 1H), 7.91 (d, J=8.2 Hz, 1H), 2.89 (s, 3H), 2.83 (s, 3H). ¹⁹F NMR (376 MHz, Chloroform-d) δ ppm −68.2 (s). m/z (ESI⁺): 340.0 [M+H]⁺.

### Method Int-18

### Intermediate 32: 7-chloro-2,3-dimethyl-5-(3-(trifluoromethyl)bicyclo[1.1.1]pentan-1-yl)pyrido[3,4-b]pyrazine

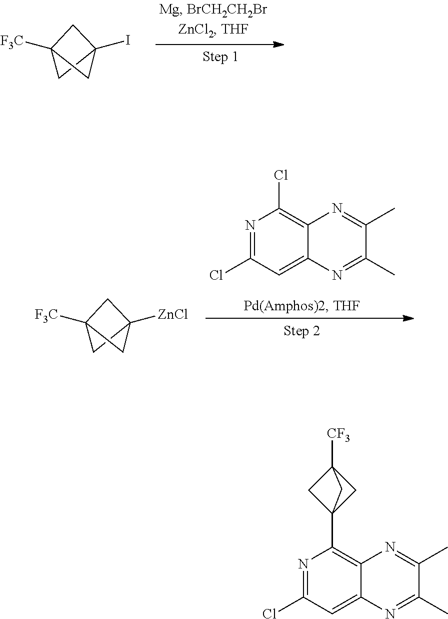

Step 1: To a flame-dried flask charged with magnesium (1.10 eq, 204 mg, 8.4 mmol) in THF (8 mL) was added 1,2-dibromoethane (5 mol %, 33 uL, 0.38 mmol). The resulting mixture was stirred for 30 minutes at r.t. before 1-iodo-3-(trifluoromethyl)bicyclo[1.1.1]pentane (1.00 eq, 2 g, 7.6 mmol) in THF (8 mL) was added. The reaction mixture was heated at 74° C. for 1 hour under vigorous stirring and cooled down to r.t. The resulting solution was added dropwise to a zinc chloride solution (0.5M in THF, 1.10 eq, 16.8 mL, 8.4 mmol) and the reaction mixture was stirred overnight at r.t. The organozinc solution was titrated using the Knochel procedure to provide a 0.12M solution of the corresponding zincate reagent (50% yield).

Step 2: In a flame-dried flask was added 5,7-dichloro-2,3-dimethyl-pyrido[3,4-b]pyrazine (0.80 eq, 701 mg, 3.1 mmol), Pd(amphos)Cl₂ (5 mol %, 136 mg, 0.19 mmol) and THF (7.7 mL). The reaction mixture was degassed for 5 minutes under N₂ and the solution of 3-(trifluoromethyl)-1-bicyclo[1.1.1]pentanyl zinc chloride (1.00 eq, 31 mL, 3.84 mmol) was added dropwise. The reaction mixture was stirred at 45° C. overnight. The reaction mixture was cooled to r.t. and the solvent was removed in vacuo. The residue was taken up in DCM (80 mL) and washed with H₂O (40 mL) and HCl (1 M, 15 mL). The aqueous phase was extracted with DCM (3×25 mL) and the combined organic phases were washed with brine, dried over MgSO₄ and the volatiles were removed in vacuo. The crude material was purified by flash chromatography (Isco RediSep® column 25 g, using a gradient from 100% DCM to 10% MeOH in DCM) to give the titled product 7-chloro-2,3-dimethyl-5-[3-(trifluoromethyl)-1-bicyclo[1.1.1]pentanyl]pyrido[3,4-b]pyrazine (490 mg, 1.50 mmol, 39%) as a white solid. ¹H NMR (400 MHz, CDCl₃) δ ppm 7.75 (s, 1H), 2.75 (s, 3H), 2.74 (s, 3H), 2.63 (s, 6H). ¹⁹F NMR (376 MHz, CDCl₃) δ ppm −73.0 (s). m/z (ESI+): 328.1 [M+H]⁺.

TABLE 6
Intermediates 33 through 36 were prepared following the procedure described in Method
Int-18, using 1-iodo-3-(trifluoromethyl)bicyclo[1.1.1]pentane and the starting materials indicated:

| Int # | Structure | IUPAC Name | Starting Material |
|---|---|---|---|
| 33 | 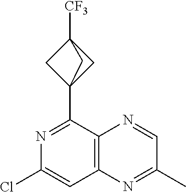 [SMILES: Cc1cnc2c(C34CC(C(F)(F)F)(C3)C4)nc(Cl)cc2n1] | 7-chloro-2-methyl-5-[3- (trifluoromethyl)-1- bicyclo[1.1.1]pentanyl]pyrido [3,4-b]pyrazine | 5,7-dichloro-2- methyl-pyrido[3,4- b]pyrazine |
| 34 | 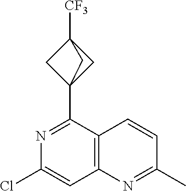 [SMILES: Cc1ccc2c(C34CC(C(F)(F)F)(C3)C4)nc(Cl)cc2n1] | 7-chloro-2-methyl-5-[3- (trifluoromethyl)-1- bicyclo[1.1.1]pentanyl]-1,6- naphthyridine | 5,7-dichloro-2- methyl-1,6- naphthyridine |
| 35 | 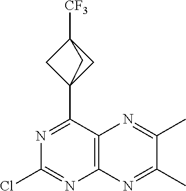 [SMILES: Cc1nc2nc(Cl)nc(C34CC(C(F)(F)F)(C3)C4)c2nc1C] | 2-chloro-6,7-dimethyl-4-[3- (trifluoromethyl)-1- bicyclo[1.1.1]pentanyl]pteridine | 2,4-dichloro-6,7- dimethyl-pteridine |
| 36 | 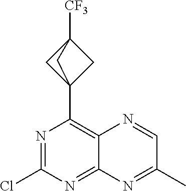 [SMILES: Cc1cnc2c(C34CC(C(F)(F)F)(C3)C4)nc(Cl)nc2n1] | 2-chloro-7-methyl-4-(3- (trifluoromethyl)bicyclo[1.1.1]pentan- 1-yl)pteridine | 2,4-dichloro-7- methylpteridine |

### Method Int-19

### Intermediate 37: 1-methyl-4-(4-(4,4,5,5-tetramethyl-1,3,2-dioxaborolan-2-yl)-5,6-dihydro-2H-pyran-2-yl)-1H-pyrazole

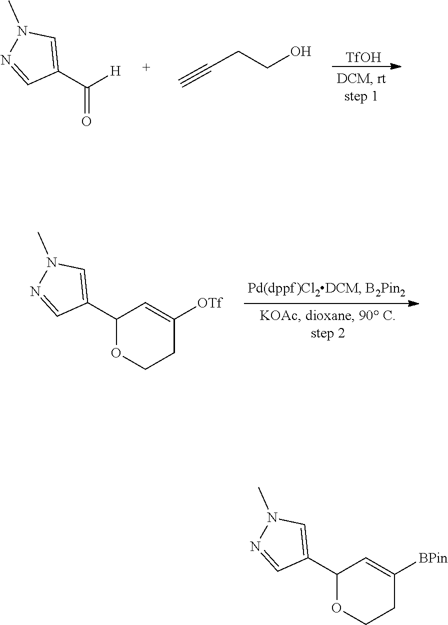

Step 1: To a 20 mL scintillation vial was charged 1-methyl-1H-pyrazole-4-carbaldehyde (200 mg, 1.816 mmol), which was purged with N₂. Then (2-hydroxyethyl)acetylene (191 mg, 206 μl, 2.72 mmol) and DCM (3.6 mL) were added. To the vial was added trifluoromethane sulfonic acid (327 mg, 194 μl, 2.180 mmol) slowly at 0° C. The reaction was warmed to room temperature after 5 min. After 5 h, additional trifluoromethane sulfonic acid (327 mg, 194 μl, 2.180 mmol) was added. After another 18 h, the crude reaction was carefully quenched with saturated NaHCO₃ solution and washed with DCM. The combined organic layers were dried over Na₂SO₄, filtered, and concentrated. The resulting crude material was absorbed onto a plug of silica gel and purified by chromatography through a Redi-Sep pre-packed silica gel column (40 g), eluting with 0% to 70% EtOAc in heptane, to provide 6-(1-methyl-1H-pyrazol-4-yl)-3,6-dihydro-2H-pyran-4-yl trifluoromethanesulfonate (227 mg, 0.727 mmol, 40% yield) as a light yellow oil. m/z (ESI, +ve ion): 313.0 [M+H]⁺. ¹H NMR (500 MHz, Chloroform-d) δ ppm 7.49 (s, 1H), 7.37 (s, 1H), 5.96 (dt, J=2.6, 1.4 Hz, 1H), 5.34 (q, J=2.6 Hz, 1H), 3.98-4.04 (m, 1H), 3.92 (s, 3H), 3.85 (ddd, J=11.5, 6.4, 5.2 Hz, 1H), 2.45-2.60 (m, 2H).

Step 2: To a 20 mL scintillation vial was charged 6-(1-methyl-1H-pyrazol-4-yl)-3,6-dihydro-2H-pyran-4-yl trifluoromethanesulfonate (227 mg, 0.727 mmol), [1,1′-bis(diphenylphosphino)ferrocene]-dichloropalladium(ii), complex with DCM (59.4 mg, 0.073 mmol), bis(pinacolato)diboron (277 mg, 1.09 mmol) and potassium acetate (285 mg, 2.91 mmol). The flask was purged with N₂ and 1,4-dioxane (2.9 mL) was added. The reaction was heated to 90° C. for 2 h and the reaction was cooled to room temperature. The reaction mixture was diluted with EtOAc and filtered through a plug of silica gel. The crude material purified by silica gel chromatography eluting with 0% to 100% EtOAc in heptane, to provide 1-methyl-4-(4-(4,4,5,5-tetramethyl-1,3,2-dioxaborolan-2-yl)-5,6-dihydro-2H-pyran-2-yl)-1H-pyrazole (87 mg, 0.30 mmol, 41% yield) as a red oil. m/z (ESI, +ve ion): 291.2 [M+H]⁺. ¹H NMR (500 MHz, Chloroform-d) δ ppm 7.48 (s, 1H), 7.36 (s, 1H), 6.61 (q, J=1.9 Hz, 1H), 5.20 (q, J=2.6 Hz, 1H), 3.89-3.93 (m, 1H), 3.89 (s, 3H), 3.71-3.78 (m, 1H), 2.28-2.39 (m, 1H), 2.17-2.27 (m, 1H), 1.30 (s, 12H).

TABLE 7
Intermediates 38-40, 44-49, 51, and 54-56 were prepared following the procedure
described in Method Int-19, starting from (2-hydroxyethyl)acetylene and the noted starting
material as follows:

| Int # | Structure | Name | Starting Material |
|---|---|---|---|
| 38 | 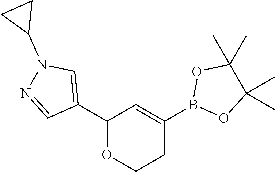 [SMILES: CC1(C)OB(C2=CC(c3cnn(C4CC4)c3)OCC2)OC1(C)C] | 1-cyclopropyl-4-(4-(4,4,5,5- tetramethyl-1,3,2-dioxaborolan- 2-yl)-5,6-dihydro-2H-pyran- 2-yl)-1H-pyrazole | 1-cyclopropyl-1H- pyrazole-4-carbaldehyde |
| 39 | 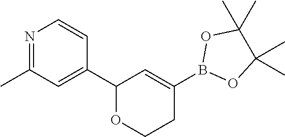 | 2-methyl-4-(4-(4,4,5,5- tetramethyl-1,3,2-dioxaborolan- 2-yl)-5,6-dihydro-2H-pyran- 2-yl)pyridine | 2-methylisonicotinaldehyde |
| 40 | 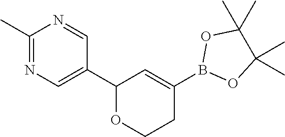 | 2-methyl-5-(4-(4,4,5,5- tetramethyl-1,3,2-dioxaborolan- 2-yl)-5,6-dihydro-2H-pyran- 2-yl)pyrimidine | 2-methylpyrimidine-5- carbaldehyde |
| 44 | 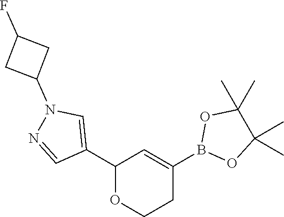 [SMILES: CC1(C)OB(C2=CC(c3cnn(C4CC(F)C4)c3)OCC2)OC1(C)C] | 1-(3-fluorocyclobutyl)-4-(4- (4,4,5,5-tetramethyl-1,3,2- dioxaborolan-2-yl)-5,6- dihydro-2H-pyran-2-yl)-1H- pyrazole | 1-(3-fluorocyclobutyl)-1H- pyrazole-4-carbaldehyde |
| 45 | 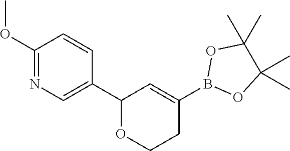 [SMILES: COc1ccc(C2C=C(B3OC(C)(C)C(C)(C)O3)CCO2)cn1] | 2-methoxy-5-(4-(4,4,5,5- tetramethyl-1,3,2-dioxaborolan- 2-yl)-5,6-dihydro-2H-pyran- 2-yl)pyridine | 6-methoxypyridine-3- carbaldehyde |
| 46 | 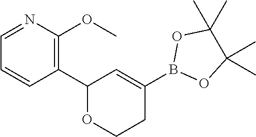 [SMILES: COc1ncccc1C1C=C(B2OC(C)(C)C(C)(C)O2)CCO1] | 2-methoxy-3-(4-(4,4,5,5- tetramethyl-1,3,2-dioxaborolan- 2-yl)-5,6-dihydro-2H-pyran- 2-yl)pyridine | 2-methoxypyridine-3- carbaldehyde |
| 47 | 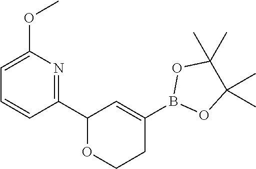 [SMILES: COc1cccc(C2C=C(B3OC(C)(C)C(C)(C)O3)CCO2)n1] | 2-methoxy-6-(4-(4,4,5,5- tetramethyl-1,3,2-dioxaborolan- 2-yl)-5,6-dihydro-2H-pyran- 2-yl)pyridine | 6-methoxypyridine-2- carbaldehyde |
| 48 | 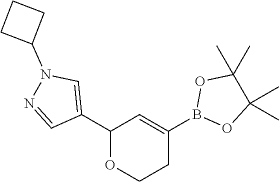 [SMILES: CC1(C)OB(C2=CC(c3cnn(C4CCC4)c3)OCC2)OC1(C)C] | 1-cyclobutyl-4-(4-(4,4,5,5- tetramethyl-1,3,2-dioxaborolan- 2-yl)-5,6-dihydro-2H-pyran- 2-yl)-1H-pyrazole | 1-cyclobutylpyrazole-4- carbaldehyde |
| 49 | 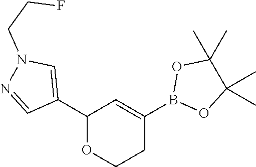 [SMILES: CC1(C)OB(C2=CC(c3cnn(CCF)c3)OCC2)OC1(C)C] | 1-(2-fluoroethyl)-4-(4-(4,4,5,5- tetramethyl-1,3,2-dioxaborolan- 2-yl)-5,6-dihydro-2H-pyran- 2-yl)-1H-pyrazole | 1-(2-fluoroethyl)pyrazole- 4-carbaldehyde |
| 51 | 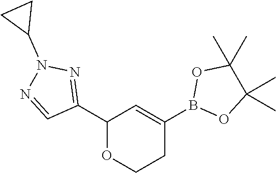 [SMILES: CC1(C)OB(C2=CC(c3cnn(C4CC4)n3)OCC2)OC1(C)C] | 2-cyclopropyl-4-(4-(4,4,5,5- tetramethyl-1,3,2-dioxaborolan- 2-yl)-5,6-dihydro-2H-pyran- 2-yl)-2H-1,2,3-triazole | 2-cyclopropyltriazole-4- carbaldehyde |
| 54 | 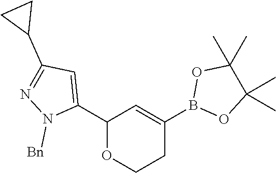 [SMILES: CC1(C)OB(C2=CC(c3cc(C4CC4)nn3Cc3ccccc3)OCC2)OC1(C)C] | 1-benzyl-3-cyclopropyl-5-(4- (4,4,5,5-tetramethyl-1,3,2- dioxaborolan-2-yl)-5,6- dihydro-2H-pyran-2-yl)-4,5- dihydro-1H-pyrazole | 1-benzyl-3-cyclopropyl- 1H-pyrazole-5- carbaldehyde |
| 55 | 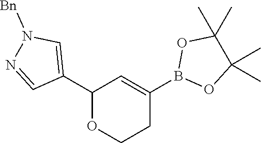 [SMILES: CC1(C)OB(C2=CC(c3cnn(Cc4ccccc4)c3)OCC2)OC1(C)C] | 1-benzyl-4-(4-(4,4,5,5- tetramethyl-1,3,2-dioxaborolan- 2-yl)-5,6-dihydro-2H-pyran- 2-yl)-1H-pyrazole | 1-benzyl-1H-pyrazole-4- carbaldehyde |
| 56 | 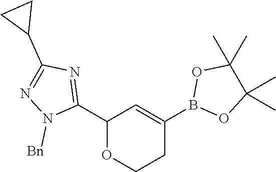 [SMILES: CC1(C)OB(C2=CC(c3nc(C4CC4)nn3Cc3ccccc3)OCC2)OC1(C)C] | 1-benzyl-3-cyclopropyl-5-(4- (4,4,5,5-tetramethyl-1,3,2- dioxaborolan-2-yl)-5,6- dihydro-2H-pyran-2-yl)-1H- 1,2,4-triazole | 2-benzyl-5-cyclopropyl- 1,2,4-triazole-3- carbaldehyde |

### Method Int-20

### Intermediate 41: 5,7-dichloro-2,3-dimethylpyrido[3,4-b]pyrazine

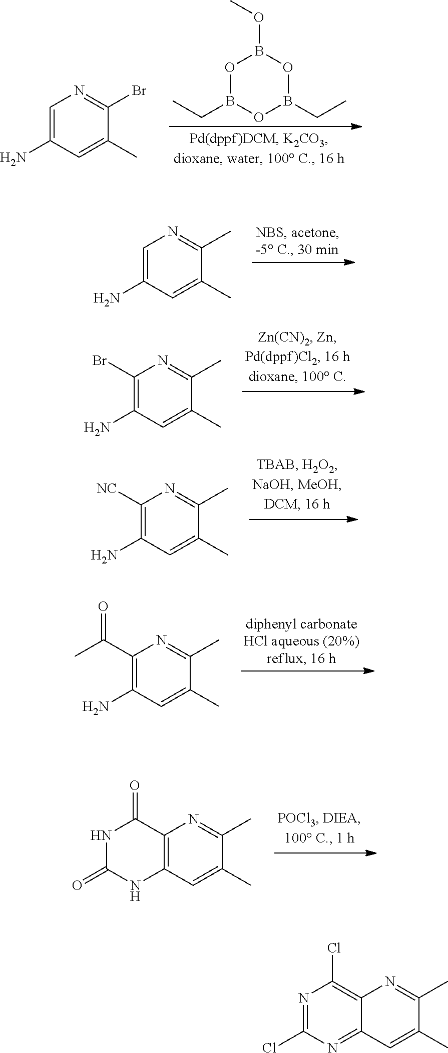

Step 1: To a solution of 6-bromo-5-methylpyridin-3-amine (5 g, 26.9 mmol) in 1,4-dioxane (50 mL) and water (5 mL) was added 2,4,6-trimethoxy-1,3,5,2,4,6-trioxatriborinane (5 g, 28.7 mmol) and potassium carbonate (11.1 mg, 80.4 mmol) and the reaction mixture was purged with nitrogen. Then [1,1′-bis(diphenylphosphino) ferrocene]dichloropalladium(II) (2.2 g, 2.7 mmol) was added and the reaction mixture was heated at 100° C. overnight. The reaction mixture was cooled to r.t and diluted with water, then extracted with DCM (200 mL*3). The combined organic layers were dried over anhydrous sodium sulfate, concentrated under reduced pressure to get a crude residue. The residue was purified by column chromatography on silica gel (PE/EtOAc=1/1) to afford 5,6-dimethylpyridin-3-amine (1.8 g, 28%) as a yellow solid. LCMS: [M+H]⁺=123.0; Retention time=1.18 min.

Step 2: To a solution of 5,6-dimethylpyridin-3-amine (0.95 g, 7.8 mmol) in acetone (20 mL) was added NBS (1.39 g, 7.8 mmol) dropwise at −5° C. and the reaction mixture was stirred for 30 min at room temperature. After completion, the reaction was quenched with water (50 mL). The aqueous layer was extracted with DCM (100 mL*3). The combined organic layer was dried over anhydrous sodium sulfate, concentrated under reduced pressure to get a crude residue. The residue was purified by column chromatography on silica gel (PE/EtOAc=10/1) to afford 2-bromo-5,6-dimethylpyridin-3-amine (1 g, 63%) as a yellow solid. LCMS: [M+H]⁺=203.0; Retention time=1.43 min.

Step 3: To a solution of 2-bromo-5,6-dimethylpyridin-3-amine (1.5 g, 7.5 mmol) in 1,4-dioxane (20 mL) was added zinc cyanide (1.8 g, 15.4 mmol) and zinc powder (0.2 g, 3.1 mmol). The reaction mixture was purged with nitrogen. Then [1,1′-bis(diphenylphosphino)ferrocene]dichloropalladium(II) (0.6 g, 0.74 mmol) was added. The reaction mixture was heated at 100° C. overnight. The reaction mixture was cooled to RT and diluted with water, and then extracted with EtOAc (100 mL*3). The combined organic layer was dried over anhydrous sodium sulfate, concentrated under reduced pressure to get a crude residue. The residue was purified by column chromatography on silica gel (PE/EtOAc=1/1) to afford 3-amino-5,6-dimethylpicolinonitrile (300 mg, 27%) as a yellow solid. LCMS: [M+H]⁺=148.0; Retention time=1.35 min.

Step 4: A mixture of 3-amino-5,6-dimethyl picolinonitrile (300 mg, 2 mmol) in methanol/dichloromethane (1/2, 6 mL) was treated with tetrabutylammonium bromide (217 mg, 0.67 mmol) and 30% aq. hydrogen peroxide (2.1 mL). The reaction was cooled 0° C. and 5 N aq. NaOH solution (6.1 mL) was added. After the addition was complete, the reaction mixture solidified. Additional methanol/dichloromethane (1:2 by volume, 6 mL) was added to dissolve the solids. The reaction was allowed to warm to r.t and was stirred overnight. After completion, the aqueous layer was extracted with ethyl acetate (50 mL*3). The combined organic layer was dried over anhydrous sodium sulfate, concentrated under reduced pressure to get a crude residue. The residue was triturated in MeOH to afford 3-amino-5,6-dimethylpicolinamide (300 mg, 89%) as a yellow solid. LCMS: [M+H]⁺=166.0; Retention time=1.39 min.

Step 5: To a solution of 3-amino-5,6-dimethylpicolinamide (1.0 eq, 500 mg, 2.99 mmol) in hydrochloric acid (20%, 10 mL) was added diphenyl carbonate (1.20 eq, 778.2 mg, 3.64 mmol). The resulting solution was heated to reflux for 3 hours. The reaction mixture was cooled and filtered. The filtrate was concentrated under vacuum. The residue was diluted with water (100 mL) and adjusted to pH=10 with aqueous ammonia (25%). The precipitate was collected by filtration, then washed with water, ethanol and ether and finally dried to give a crude product. The crude solid was triturated with THF/MeOH/EA=1/1/1 by volume, filtered and dried to yield 6,7-dimethylpyrido[3,2-d]pyrimidine-2,4(1H,3H)-dione (350 mg, 1.83 mmol, 61% yield). LCMS: [M+H]⁺=192.1; Retention time: 1.25 min.

Step 6: To a solution of 6,7-dimethyl-1H-pyrido[3,2-d]pyrimidine-2,4-dione (1.00 eq, 1100 mg, 5.75 mmol) in phosphorus oxychloride (20.0 eq, 11 mL, 115 mmol) was added dropwise N,N-diisopropylethylamine (5.00 eq, 5.0 mL, 28.8 mmol) at rt. The mixture was stirred at 100° C. for 1 h under nitrogen. After cooling down, the reaction mixture was concentrated under reduced pressure to remove the phosphorus oxychloride, and then the residue was treated with water (50 mL) and extracted with ethyl acetate (50 mL*3). The organic layers were combined, washed with brine (50 mL), dried with anhydrous sodium sulfate, filtered and concentrated under reduced pressure. The residue was purified by column chromatography (0-50% ethyl acetate in petroleum ether) to give 2,4-dichloro-6,7-dimethyl-pyrido[3,2-d]pyrimidine (1.17 g, 4.76 mmol, 82.71% yield) as a white solid. LCMS: [M+H]⁺=228.1; Retention time: 1.99 min.

### Method Int-21

### Intermediate 50: 2-cyclopropyltriazole-4-carbaldehyde

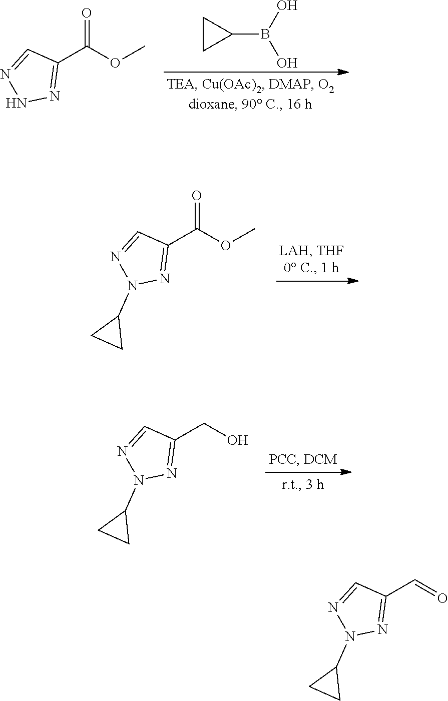

Step 1: A mixture of methyl 2H-triazole-4-carboxylate (1.00 eq, 3000 mg, 23.6 mmol), cyclopropylboronic acid (2.00 eq, 4055 mg, 47.2 mmol), Cu(OAc)₂ (1.00 eq, 4272 mg, 23.6 mmol), and DMAP (3.00 eq, 8639 mg, 70.8 mmol) in 1,4-dioxane (110 mL) was stirred at 90° C. for 16 hrs. The reaction was concentrated, and purified by column chromatography, eluting with DCM to get methyl 2-cyclopropyltriazole-4-carboxylate (1380 mg, 8.26 mmol, 34.97% yield) as a white solid. LCMS: Rt: 1.66 min; [M+H]⁺=168.0; 90.54% purity at 214 nm.

Step 2: To a solution of methyl 2-cyclopropyltriazole-4-carboxylate (1.00 eq, 1.38 g, 8.26 mmol) in THF (28 mL) was added LiAlH₄ (2.50 eq, 21 mL, 20.6 mmol) at 0° C. The reaction was stirred at 0° C. for 1 h under N₂. The reaction was quenched by addition of 0.8 mL of water dropwise at 0° C., followed by 0.8 mL of aq. NaOH (10%), and 2.4 mL of water. The mixture was stirred at r.t for 10 min and MgSO₄ was added. After stirring for an additional 10 min, the mixture was filtered, and the filtrate was concentrated. The crude product was purified by column chromatography eluting with 30% EtOAc in PE to afford (2-cyclopropyltriazol-4-yl)methanol (1050 mg, 7.55 mmol, 91.40% yield) as a white solid. LCMS: Rt: 1.28 min; [M+H]⁺=140.3.

Step 3: To a solution of (2-cyclopropyltriazol-4-yl)methanol (1.00 eq, 950 mg, 6.83 mmol) in DCM (34 mL) was added PCC (3.30 eq, 4844 mg, 22.5 mmol), and the reaction mixture was stirred at 25° C. for 3 h. The mixture was filtered, and the filtrate was concentrated. The crude product was purified by flash column chromatography eluting with 20% EtOAc in PE to afford 2-cyclopropyltriazole-4-carbaldehyde (518 mg, 3.78 mmol, 55.33% yield) as a colorless oil. LC-MS: Rt: 1.58 min; [M+H]⁺=285.3.

### Method Int-22

### Intermediate 53: 1-benzyl-3-cyclopropyl-1H-pyrazole-5-carbaldehyde

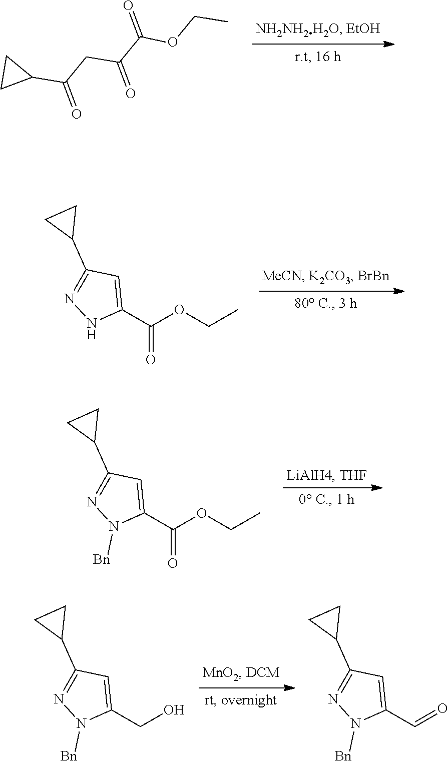 [SMILES: CCOC(=O)C(=O)CC(=O)C1CC1 ; CCOC(=O)c1cc(C2CC2)n[nH]1 ; CCOC(=O)c1cc(C2CC2)nn1Cc1ccccc1 ; OCc1cc(C2CC2)nn1Cc1ccccc1 ; O=Cc1cc(C2CC2)nn1Cc1ccccc1]

Step 1: A solution of ethyl 4-cyclopropyl-2,4-dioxo-butanoate (1.00 eq, 5.00 g, 27.1 mmol) and hydrazinium hydroxide solution (1.00 eq, 1359 mg, 27.1 mmol) in ethanol (30 mL) was stirred at room temperature for 16 hrs. The mixture was concentrated to get the ethyl 3-cyclopropyl-1H-pyrazole-5-carboxylate (4.50 g, 25.0 mmol, 91.99% yield) as a white solid. LCMS: Rt: 1.67 min; [M+H]⁺=180.9; 85.37% purity at 254 nm.

Step 2: To a solution of ethyl 3-cyclopropyl-1H-pyrazole-5-carboxylate (1.00 eq, 4.50 g, 25.0 mmol) in acetonitrile (100 mL) was added potassium carbonate (3.00 eq, 10.35 g, 75.0 mmol) and bromomethylbenzene (1.50 eq, 6.38 g, 37.5 mmol). The reaction was stirred at 80° C. for 3 h. The reaction was filtered, and the filtrate was concentrated to a residue. The residue was purified by flash column chromatography eluting with 20% EtOAc in petroleum ether. The desired fractions were concentrated to dryness in vacuo to get ethyl 2-benzyl-5-cyclopropyl-pyrazole-3-carboxylate (5.50 g, 20.3 mmol, 73.33% yield) as a colorless oil. LC-MS: Rt: 2.08 min; [M+H]⁺=271.2.

Step 3: To a solution of ethyl 2-benzyl-5-cyclopropyl-pyrazole-3-carboxylate (1.00 eq, 5.50 g, 20.3 mmol) in THF (50 mL) was added dropwise lithium aluminum hydride (2.50 eq, 51 mL, 50.9 mmol) at 0° C. under nitrogen. The mixture was allowed to slowly warm to room temperature and stirred for 1 h. The reaction was quenched by addition of NH₄Cl (sat.aq). The reaction mixture was taken up in EtOAc (400 mL) and the organics were washed with 2*100 mL water and then 100 mL of saturated brine solution. The organics were then separated and dried with MgSO₄ and then concentrated to a residue. The crude product was then purified by flash column chromatography eluting with 50% EtOAc in petroleum ether. The desired fractions were concentrated to dryness in vacuo to get (2-benzyl-5-cyclopropyl-pyrazol-3-yl)methanol (4.00 g, 17.5 mmol, 86.12% yield) as a colorless oil. LC-MS: Rt: 1.76 min; [M+H]⁺=229.2.

Step 4: To a solution of (2-benzyl-5-cyclopropyl-pyrazol-3-yl)methanol (1.00 eq, 4.00 g, 17.5 mmol) in dichloromethane (50 mL) was added manganese dioxide (10.0 eq, 15.23 g, 175 mmol) at 0° C. The mixture was stirred at r.t for 16 hrs. The reaction mixture was filtered and concentrated to dryness and the residue was purified by flash column chromatography eluting 20% EtOAc in petroleum ether. The desired fractions were concentrated to dryness in vacuo to get 2-benzyl-5-cyclopropyl-pyrazole-3-carbaldehyde (3.80 g, 16.8 mmol, 95.85% yield) as colorless oil. LC-MS: Rt: 2.04 min, 2.12 min; [M+H]⁺=227.2; 97.58% purity at 254 nm.

### Method Int-23

### Intermediate 57: 2-benzyl-5-cyclopropyl-1,2,4-triazole-3-carbaldehyde

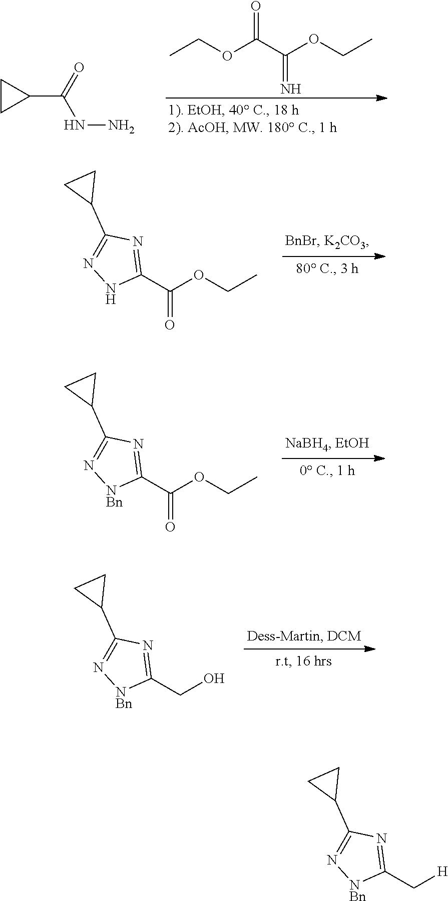

Step 1: A solution of cyclopropanecarbohydrazide (1.00 eq, 6.90 g, 68.9 mmol) and ethyl 2-ethoxy-2-imino-acetate (1.00 eq, 10.00 g, 68.9 mmol) in ethanol (100 mL) was stirred at 40° C. overnight. The reaction mixture was filtered to afford ethyl 2-[2-(cyclopropanecarbonyl)hydrazino]-2-imino-acetate (8.50 g, 42.7 mmol, 61.94% yield) as a white solid. The ethyl 2-[2-(cyclopropanecarbonyl)hydrazino]-2-imino-acetate (1.00 eq, 7.50 g, 37.6 mmol) was added to acetic acid (70 mL) and stirred at 180° C. for 1 h in a microwave. The reaction was concentrated to dryness and the residue was taken up in EtOAc (200 mL) and the organics were washed with saturated NaHCO₃ solution (100 mL*3) and brine (100 mL). The organics were then separated and dried with MgSO₄ before concentrating to a residue. The crude residue was then purified by flash column chromatography, eluting with 50% EtOAc in petroleum ether. The desired fractions were concentrated to dryness in vacuo to afford ethyl 3-cyclopropyl-1H-1,2,4-triazole-5-carboxylate (4.50 g, 24.8 mmol, 65.97% yield) as a yellow oil. LCMS: Rt: 1.41 min; [M+H]⁺=182.2; 74.72% purity at 214 nm.

Step 2: To a solution of ethyl 3-cyclopropyl-1H-1,2,4-triazole-5-carboxylate (1.00 eq, 4.50 g, 24.8 mmol) in acetonitrile (100 mL) was added potassium carbonate (3.00 eq, 10.30 g, 74.5 mmol) and bromomethylbenzene (1.50 eq, 6.37 g, 37.3 mmol). The reaction was stirred at 80° C. for 3 h. The reaction was filtered, and the filtrate was concentrated to dryness and the residue was purified by flash column chromatography eluting with 20% EtOAc in petroleum ether. The desired fractions were concentrated to dryness in vacuo to afford ethyl 2-benzyl-5-cyclopropyl-1,2,4-triazole-3-carboxylate (5.20 g, 19.2 mmol, 77.17% yield) as colorless oil. LC-MS: Rt: 2.03 min; [M+H]⁺=272.3; 92.05% purity at 214 nm.

Step 3: To a solution of ethyl 2-benzyl-5-cyclopropyl-1,2,4-triazole-3-carboxylate (1.00 eq, 5.20 g, 19.2 mmol) in ethanol (100 mL) was added sodium cyanoborohydride (2.50 eq, 3.01 g, 48.0 mmol) dropwise at 0° C. under nitrogen. The reaction was stirred at 0° C. for 1 h. The reaction was concentrated to dryness and the residue was taken up in EtOAc (500 mL) and the organics washed with water (100 mL*3) and brine (100 mL). The organics were then separated and dried with MgSO₄ before concentration to dryness. The crude material was then purified by flash column chromatography eluting with 50% dichloromethane in methanol. The desired fractions were concentrated to dryness in vacuo to afford (2-benzyl-5-cyclopropyl-1,2,4-triazol-3-yl)methanol (3.68 g, 16.1 mmol, 83.74% yield). LC-MS: Rt: 1.60 min, 1.64 min; [M+H]⁺=230.3; 89.22% purity at 214 nm.

Step 4: To a solution of (2-benzyl-5-cyclopropyl-1,2,4-triazol-3-yl)methanol (1.00 eq, 3.10 g, 13.5 mmol) in DCM (200 mL) was added Dess-Martin periodinane (2.00 eq, 11.47 g, 27.0 mmol) at 0° C. in batches. The mixture was stirred at r.t for 16 hrs. The reaction was filtered and the filter cake was washed with DCM (50 mL*2). The filtrate was concentrated to remove DCM, quenched with saturated NaHCO₃ solution (100 mL), and extracted with EtOAc (100 mL*3). The organic layers were combined, washed with brine (100 mL), dried with anhydrous sodium sulfate, filtered and concentrated under reduced pressure. The residue was purified by column chromatography (0-30% ethyl acetate in petroleum ether) to afford 2-benzyl-5-cyclopropyl-1,2,4-triazole-3-carbaldehyde (2.70 g, 11.9 mmol, 87.87% yield) as a yellow oil. LC-MS: Rt=1.82 min; [M+H]⁺=228.1; 100% purity at 254 nm.

### Example A2: Synthesis of Exemplary Compounds

### Method 1

### Example 4: 6,7-dimethyl-2-((2R,4S)-2-(2-methylpyridin-4-yl)tetrahydro-2H-pyran-4-yl)-4-(6-(trifluoromethyl)pyridin-3-yl)pteridine

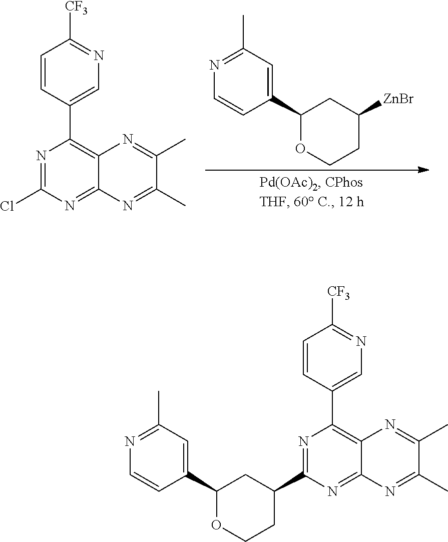

A flame-dried microwave vial under argon was charged with 2-chloro-6,7-dimethyl-4-(6-(trifluoromethyl)pyridin-3-yl)pteridine (105 mg, 308 μmol), CPhos (25.8 mg, 59.0 μmol) and THF (2.70 mL). The reaction mixture was degassed for 5 min with argon then ((2S,4S)-2-(2-methylpyridin-4-yl)tetrahydro-2H-pyran-4-yl)zinc(II) bromide (1.54 mL, 384 μmol) was added dropwise. The reaction vial was sealed and immersed in a pre-heated oil bath at 60° C. The reaction was stirred overnight at 60° C. When the conversion was judged complete by LCMS, the reaction mixture was cooled down to r.t., diluted with EtOAc (5 mL) and passed through a silica pad (1 cm). The silica was rinsed with EtOAc (10 mL) followed by 10% MeOH in CH₂Cl₂. The volatiles were removed in vacuo and the crude material was purified by flash chromatography (Isco RediSep® column 24 g, using a gradient from 50% EtOAc in CH₂Cl₂ to 100% EtOAc followed by 5 CV at 10% MeOH in CH₂Cl₂). The selected fractions were evaporated to yield the desired 6,7-dimethyl-2-((2R,4S)-2-(2-methylpyridin-4-yl)tetrahydro-2H-pyran-4-yl)-4-(6-(trifluoromethyl)pyridin-3-yl)pteridine ((57.2 mg, 39%)). LCMS: m/z (ESI) [M+H]⁺ 481.20, t_{R}=1.302 min. ¹H NMR Major dia. (DMSO-d₆, 400 MHz): δ_{H} 1.77 (1H, q, J 12.2 Hz), 2.06-1.93 (1H, m), 2.16 (1H, d, J=13.1 Hz), 2.44 (3H, s), 2.73 (3H, s), 2.78 (3H, s), 3.59 (1H, t, J 11.6 Hz), 3.80 (1H, t, J 11.8 Hz), 4.25 (1H, dd, J=11.3, 4.2 Hz), 4.62 (1H, d, J=11.2 Hz), 7.19 (1H, d, J=5.3 Hz), 7.27 (1H, s), 8.15 (1H, d, J=8.3 Hz), 8.37 (1H, d, J=5.3 Hz), 8.90 (1H, d, J=8.2 Hz), 9.59 (1H, s).

### Method 2

### Example 15: 4-(4-chloro-2-fluorophenyl)-2-((2S,4S)-2-(1-cyclopropyl-1H-pyrazol-4-yl)tetrahydro-2H-pyran-4-yl)-6,7-dimethylpteridine

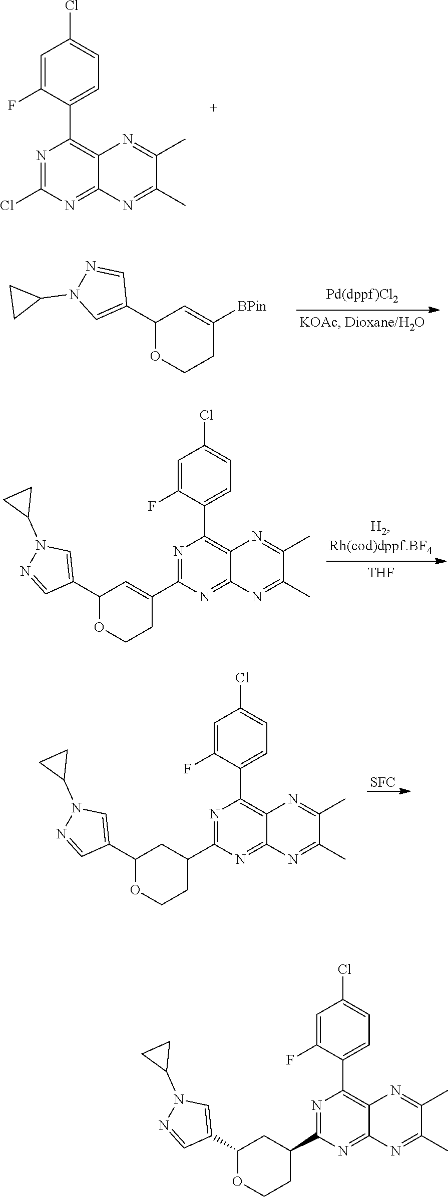

Step 1: To a solution of 2-chloro-4-(4-chloro-2-fluorophenyl)-6,7-dimethylpteridine (735 mg, 2.28 mmol, 1.0 eq) in 1,2-dioxane (8 mL) and H₂O (2 mL) was added 1-cyclopropyl-4-(4-(4,4,5,5-tetramethyl-1,3,2-dioxaborolan-2-yl)-5,6-dihydro-2H-pyran-2-yl)-1H-pyrazole (864 mg, 2.74 mmol, 1.2 eq) and potassium acetate (670 mg, 6.84 mmol, 3.0 eq). The mixture was purged with N₂ for 15 min. Then PdCl₂(dppf)-CH₂Cl₂ (93 mg, 0.114 mmol, 0.05 eq) was added. The reaction was stirred at 80° C. overnight. The reaction was filtered over a celite bed under vacuum, washed with dioxane and concentrated. The residue was purified through a silica column with 10-100% ethyl acetate (EA) in petroleum ether (PE) to give 4-(4-chloro-2-fluorophenyl)-2-(6-(1-cyclopropyl-1H-pyrazol-4-yl)-3,6-dihydro-2H-pyran-4-yl)-6,7-dimethylpteridine (663 mg, 1.39 mmol) as brown solid. LCMS: (M+H)⁺=477.1. Purity=95.03% (214 nm).

Step 2: To a round-bottomed flask was added 4-(4-chloro-2-fluorophenyl)-2-(6-(1-cyclopropyl-1H-pyrazol-4-yl)-3,6-dihydro-2H-pyran-4-yl)-6,7-dimethylpteridine (663 mg, 1.39 mmol) in THF (6 mL). The mixture was degassed with nitrogen for 5 min, then [Rh(dppf)(COD)]BF₄ (202 mg, 0.28 mmol, 0.2 eq) was added and the reaction mixture was stirred under hydrogen gas atmosphere (balloon pressure) at rt for 2 h. The solvent was evaporated under reduced vacuum and the residue was purified by silica column (100) with 5%-100% ether acetate in petroleum ether to 4-(4-chloro-2-fluorophenyl)-2-(2-(1-cyclopropyl-1H-pyrazol-4-yl)tetrahydro-2H-pyran-4-yl)-6,7-dimethylpteridine (500 mg, 1.046 mmol) as a brown solid.

Step 3: The mixture of diastereomers (500 mg, 1.046 mmol) were separated by chiral SFC eluting with CO₂/MeOH (0.2% Methanol Ammonia)=65/35 over a Daicel® AD column (20×250 mm, 10 μm) to give the four diastereomers of 4-(4-chloro-2-fluorophenyl)-2-(2-(1-cyclopropyl-1H-pyrazol-4-yl)tetrahydro-2H-pyran-4-yl)-6,7-dimethylpteridine.

### Method 3

### Example 36: 2-(1-cyclopropylpyrazol-4-yl)-4-[5-(2,4-difluorophenyl)-2-methyl-pyrido[3,4-b]pyrazin-7-yl]morpholine

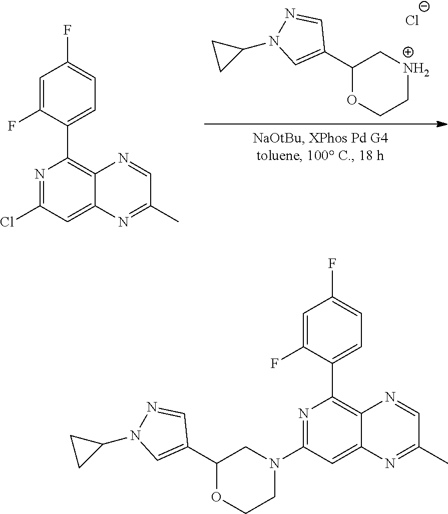 [SMILES: Cc1cnc2c(-c3ccc(F)cc3F)nc(Cl)cc2n1 ; c1nn(C2CC2)cc1C1C[NH2+]CCO1 ; [Cl-] ; Cc1cnc2c(-c3ccc(F)cc3F)nc(N3CCOC(c4cnn(C5CC5)c4)C3)cc2n1]

To a mixture of 7-chloro-5-(2,4-difluorophenyl)-2-methyl-pyrido[3,4-b]pyrazine (90 mg, 0.309 mmol), 2-(1-cyclopropylpyrazol-4-yl)morpholin-4-ium chloride (85 mg, 0.370 mmol), and sodium tert-butoxide (26 mg, 0.269 mmol) in toluene (2.5 mL) was added XPhos Pd G4 (19 mg, 0.022 mmol). The mixture was heated to 100° C. and stirred overnight. The reaction was cooled to r.t., and water was added. The solid was filtered over celite and rinsed with EtOAc. The product was extracted from the filtrate with EtOAc, and the combined organic layers were washed with brine, dried over Na₂SO₄, filtered, and concentrated in vacuo. The crude material was purified by silica gel chromatography eluting with 20-100% EtOAc in hexanes to provide the title compound 2-(1-cyclopropylpyrazol-4-yl)-4-[5-(2,4-difluorophenyl)-2-methyl-pyrido[3,4-b]pyrazin-7-yl]morpholine (65 mg, 0.138 mmol, 45% yield) as an orange solid. ¹H NMR (400 MHz, DMSO-d₆) δ ppm 8.51 (s, 1H), 7.85 (s, 1H), 7.66 (td, J=8.4, 6.6 Hz, 1H), 7.48 (s, 1H), 7.36 (td, J=9.8, 2.5 Hz, 1H), 7.23 (td, J=8.6, 2.6 Hz, 1H), 7.18 (s, 1H), 4.56 (dd, J=10.4, 2.7 Hz, 1H), 4.41 (d, J=13.2 Hz, 1H), 4.29-4.19 (m, 1H), 4.09-3.86 (m, 1H), 3.89-3.52 (m, 2H), 3.21-2.84 (m, 2H), 2.64 (s, 3H), 1.11-0.98 (m, 2H), 0.98-0.89 (m, 2H). LC/MS (ESI+) m/z=449.2 [M+H]⁺

### Method 4

### Example 41: 4-(5-(2,4-Difluorophenyl)-2,3-dimethyl-1,6-naphthyridin-7-yl)-2-(2-methylpyridin-4-yl)morpholine

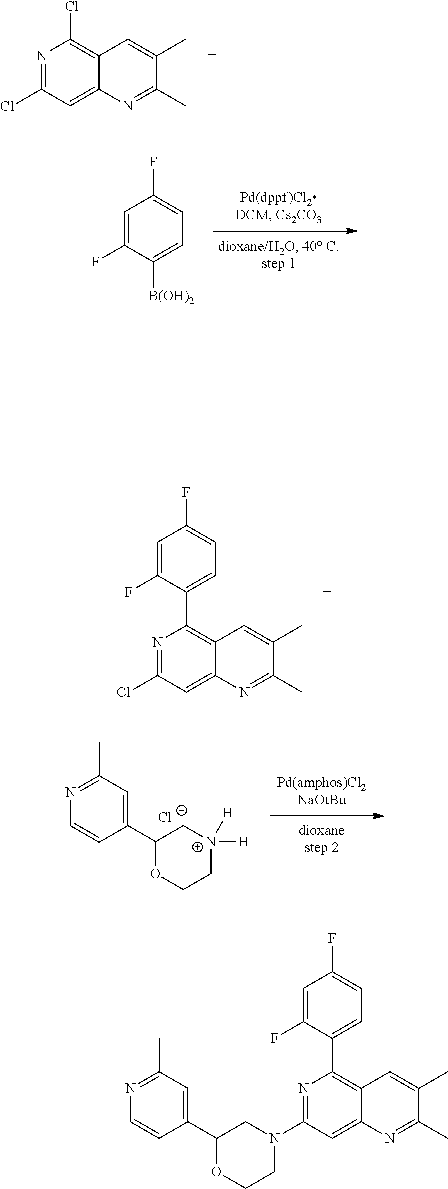 [SMILES: Cc1cc2c(Cl)nc(Cl)cc2nc1C ; OB(O)c1ccc(F)cc1F ; Cc1cc2c(-c3ccc(F)cc3F)nc(Cl)cc2nc1C ; Cc1cc(C2C[NH2+]CCO2)ccn1 ; [Cl-] ; Cc1cc(C2CN(c3cc4nc(C)c(C)cc4c(-c4ccc(F)cc4F)n3)CCO2)ccn1]

Step 1: A 50 mL microwave vial was charged with (2,4-difluorophenyl)boronic acid (556 mg, 3.52 mmol), 5,7-dichloro-2,3-dimethyl-1,6-naphthyridine (800 mg, 3.52 mmol), cesium carbonate (3.44 g, 10.6 mmol), 1,4-dioxane (16 mL) and water (4.8 mL). The reaction mixture was degassed with nitrogen for 10 min. Pd(dppf)Cl₂·CH₂Cl₂ (144 mg, 0.176 mmol) was added, and the mixture was heated at 40° C. for 1 h. The mixture was cooled to r.t., and diluted with DCM (50 mL) and water (10 mL). The aqueous layer was extracted with DCM (2×25 mL). The combined organic layers were washed with brine (10 mL), dried (Na₂SO₄) and concentrated under reduced pressure. The residue was purified by silica gel chromatography (80 g SilicaSep cartridge) using EtOAc and hexanes (30-40%) to obtain 7-chloro-5-(2,4-difluorophenyl)-2,3-dimethyl-1,6-naphthyridine (660 mg, 2.17 mmol, 62%) as a solid. ESI-MS (m/z+): 305.1 [M+H]⁺, LC-RT: 2.09 min. ¹H NMR (400 MHz, CDCl₃) δ ppm 7.92 (s, 1H), 7.70 (d, J=2.7 Hz, 1H), 7.62-7.53 (m, 1H), 7.13-7.05 (m, 1H), 7.04-6.95 (m, 1H), 2.73 (s, 3H), 2.43 (s, 3H). ¹⁹F NMR (376 MHz, CDCl₃) δ ppm −107.43 (s), -109.37 (s).

Step 2: A mixture of 7-chloro-5-(2,4-difluorophenyl)-2,3-dimethyl-1,6-naphthyridine (50 mg, 0.164 mmol), 2-(2-methyl-4-pyridyl)morpholin-4-ium chloride (36 mg, 0.169 mmol), sodium tert-butoxide (63 mg, 0.658 mmol), and Pd(amphos)Cl₂ (12 mg, 0.0164 mmol) in 10 mL microwave vial was subjected to three cycles of vacuum/nitrogen fill. 1,4-Dioxane (2.5 mL) was added, and the mixture was stirred at 80° C. for 5 h. The mixture was cooled to r.t., and diluted with EtOAc (50 mL) and water (20 mL). The layers were separated, and the aqueous layer was extracted with EtOAc (2×50 mL). The combined organic layers were washed with brine (10 mL), dried over Na₂SO₄, and concentrated in vacuo. The residue was purified by silica gel chromatography (SilicaSep 24 g cartridge) using MeOH and dichloromethane (20-30%) to obtain an oil which was further purified by reverse phase chromatography on ACCQ prep HPLC (Gemini 150×30 mm C18 column) using acetonitrile and water (80-90%) to obtain 4-[5-(2,4-difluorophenyl)-2,3-dimethyl-1,6-naphthyridin-7-yl]-2-(2-methyl-4-pyridyl)morpholine (19 mg, 0.0410 mmol, 25%) as a yellow solid. ESI-MS (m/z+): 447.20 [M+H]⁺, LC-RT: 2.313 min. ¹H NMR (400 MHz, CD2Cl2) δ ppm 8.45 (d, J=5.2 Hz, 1H), 7.57-7.49 (m, 2H), 7.25 (s, 1H), 7.18 (d, J=5.1 Hz, 1H), 7.12-6.98 (m, 3H), 4.64 (dd, J=10.4, 2.5 Hz, 1H), 4.46 (d, J=12.4 Hz, 1H), 4.25-4.16 (m, 2H), 3.95-3.86 (m, 1H), 3.19-3.09 (m, 1H), 2.85 (dd, J=12.7, 10.6 Hz, 1H), 2.62 (s, 3H), 2.54 (s, 3H), 2.32 (s, 3H). ¹⁹F NMR (376 MHz, CD₂Cl₂) δ ppm −109.71 (s), -110.69 (s).

### Method 5

### Examples 56 and 57: 2-(1-cyclopropyl-1H-pyrazol-4-yl)-4-(5-(2,4-difluorophenyl)-2-methylpyrido[3,4-b]pyrazin-7-yl)-6-methylmorpholine

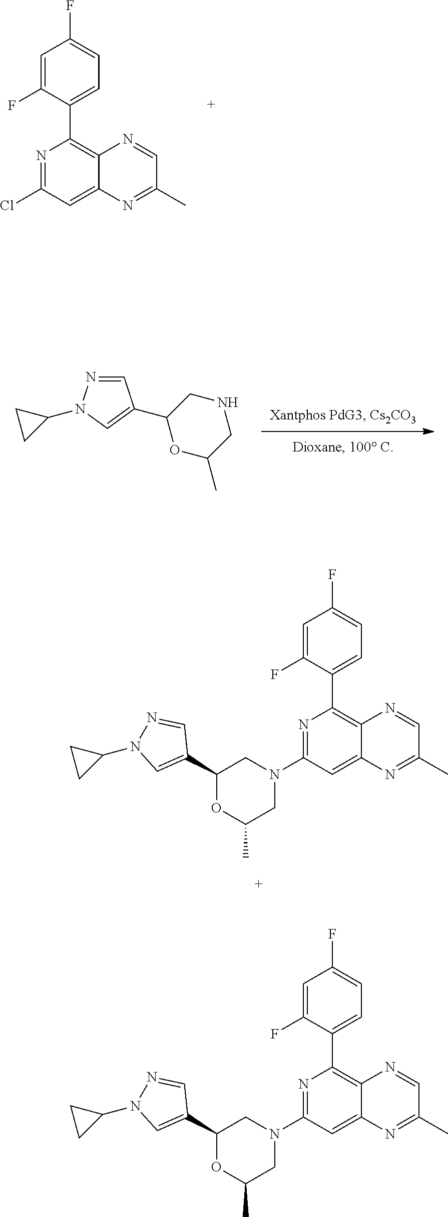 [SMILES: Cc1cnc2c(-c3ccc(F)cc3F)nc(Cl)cc2n1 ; CC1CNCC(c2cnn(C3CC3)c2)O1 ; Cc1cnc2c(-c3ccc(F)cc3F)nc(N3C[C@H](C)O[C@@H](c4cnn(C5CC5)c4)C3)cc2n1 ; Cc1cnc2c(-c3ccc(F)cc3F)nc(N3C[C@@H](C)O[C@@H](c4cnn(C5CC5)c4)C3)cc2n1]

To a suspension of 7-chloro-5-(2,4-difluorophenyl)-2-methylpyrido[3,4-b]pyrazine (400 mg, 1.371 mmol, 1.0 eq), 2-(1-cyclopropyl-1H-pyrazol-4-yl)-6-methylmorpholine (528 mg, 1.645 mmol, 1.2 eq), and Cs₂CO₃ (2.233 g, 6.855 mmol, 5.0 eq) in anhydrous dioxane (15 mL) was added Xantphos PdG3 (195 mg, 0.206 mmol, 0.15 eq) under N₂ and the reaction mixture was purged with N₂ three times and stirred at 100° C. for 16 h to give a brown suspension. The reaction mixture was filtered and washed with DCM (50 mL×3), the combined filtrate was concentrated under vacuum to give a blown solid. The solid was triturated with a mixture solution of DCM (5 mL) and PE (50 mL), then washed with PE (30 mL), and the combined liquids were concentrated under vacuum to give the crude product as an orange solid. The crude product was purified by column (SiO₂, PE:EA=15:1-1:1) and prep HPLC to give the diastereomers P1 (2.6 mg) and P2 (14.4 mg) as yellow solid. The racemate product was purified by SFC (OD-H 4.6×100 cm, 5 μm column; 1% Methanol Ammonial, F=3.0 mL/min) to provide separated cis and trans products. ¹H NMR (400 MHz, CDCl₃) δ ppm 8.48 (s, 1H), 7.66-7.60 (m, 2H), 7.52 (s, 1H), 7.50 (d, J=3.1 Hz, 2H), 7.00 (s, 1H), 5.08 (s, 1H), 4.51-4.47 (m, 1H), 4.01-3.99 (m, 1H), 3.73 (d, J=9.2 Hz, 1H), 3.53 (d, J=3.6 Hz, 2H), 3.25-3.17 (m, 2H), 2.87 (s, 3H), 1.28 (d, J=6.3 Hz, 3H), 1.06 (d, J=4.0 Hz, 1H), 0.98 (d, J=5.3 Hz, 2H). LCMS: (M+H)⁺=463.

### Method 6

### Examples 69 and 70: 4-(4-(4-chloro-2-fluorophenyl)-7-methylpteridin-2-yl)-2-(1-cyclopropyl-1H-pyrazol-4-yl)-6-methylmorpholine

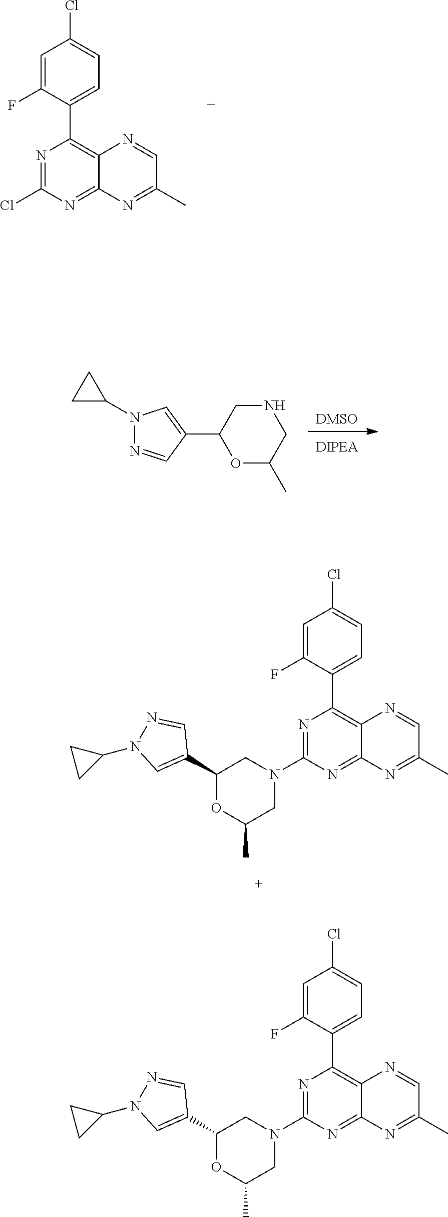

To a solution of 2-chloro-4-(4-chloro-2-fluorophenyl)-7-methylpteridine (400 mg, 1.29 mmol, 1.0 eq) in DMSO (5 mL) was added 2-(1-cyclopropyl-1H-pyrazol-4-yl)-6-methylmorpholine (348 mg, 1.68 mmol, 1.3 eq) and DIPEA (1.07 mL, 6.45 mmol, 5.0 eq). The mixture was stirred at 100° C. for 2 h. After 2 hours, LCMS showed no starting material remained. The reaction mixture was extracted with H₂O (40 mL×2) and EA (20 mL) and the organic layers were combined, dried over Na₂SO₄, and evaporated to dryness to give the crude product. The crude product was purified by prep HPLC to give the trans diastereomer (88 mg) and cis diastereomer (170 mg). The cis diastereomer mixture was separated by chiral SFC-150 eluting with CO₂/IPA (0.2% Methanol Ammonia)=65/35 over an Daicel® OD column (20×250 mm 10 μm) to give the two enantiomers Examples 117 and 118.

### Method 7

### Examples 84 and 85: 4-(4-chloro-3,5-difluoro-phenyl)-6,7-dimethyl-2-[(2R,4S)-2-(2-methyl-4-pyridyl)tetrahydropyran-4-yl]pteridine and 4-(4-chloro-3,5-difluoro-phenyl)-6,7-dimethyl-2-[(2R,4R)-2-(2-methyl-4-pyridyl)tetrahydropyran-4-yl]pteridine

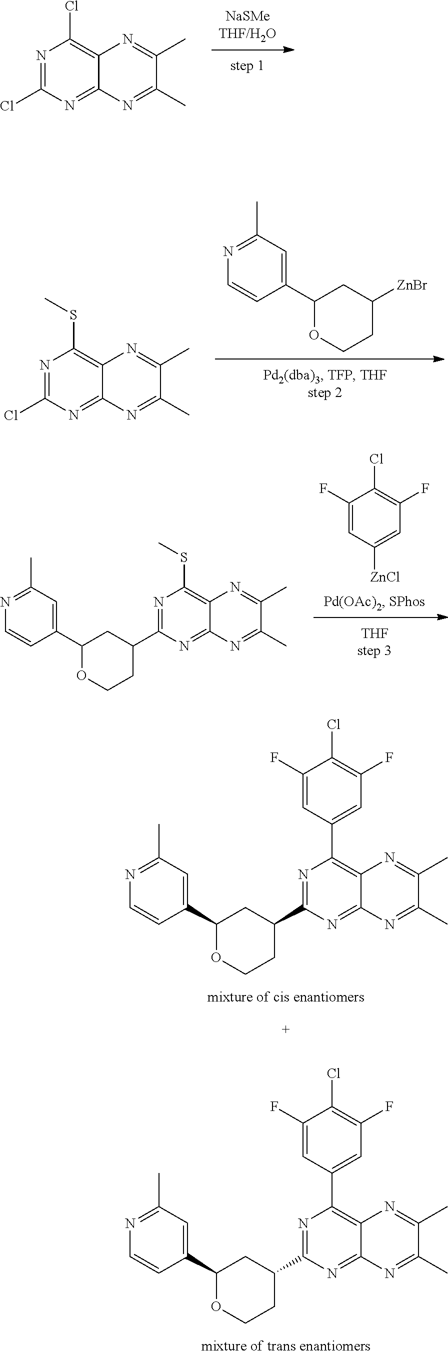

Step 1: A 100 mL round-bottom flask was charged with 2,4-dichloro-6,7-dimethyl-pteridine (3.00 g, 13.1 mmol) and THF (40 mL). The solution was cooled to −10° C. and a suspension of NaSMe (1.01 g, 14.4 mmol) in water (5 mL) was added dropwise. The reaction mixture was warmed to r.t. and stirred for 17 h. The mixture was diluted with DCM (50 mL) and water (10 mL). The aqueous layer was extracted with DCM (2×10 mL). Combined organic layer was dried over Na₂SO₄ and concentrated in vacuo. The crude residue was purified by silica gel chromatography (80 g SilicaSep column) using EtOAc and hexanes (50-60%) to obtain 2-chloro-6,7-dimethyl-4-methylsulfanyl-pteridine (1.92 g, 7.98 mmol, 61%) as a pale yellow solid. ESI-MS (m/z+): 241.0 [M+H]⁺, LC-RT: 2.907 min. ¹H NMR (400 MHz, CDCl₃) δ ppm 2.79 (s, 3H), 2.76 (s, 3H), 2.70 (s, 3H).

Step 2: A 50 mL microwave vial was charged with a solution of 2-chloro-6,7-dimethyl-4-methylsulfanyl-pteridine (600 mg, 2.49 mmol), Pd₂(dba)₃ (36 mg, 0.0626 mmol) and tri(2-furyl)phosphine (30 mg, 0.129 mmol) in THF (12 mL) and subjected to three cycles of vacuum/nitrogen fill. Bromo-[2-(2-methyl-4-pyridyl)tetrahydropyran-4-yl]zinc bromide solution (0.16 M in THF, 23 ml, 3.74 mmol) was then added dropwise at 25° C. and the mixture was stirred for 44 h. The mixture was diluted with DCM (100 mL) and sat. NaHCO₃ (20 mL). The aqueous layer was extracted with DCM (2×50 mL). The combined organic layer was washed with brine, dried over Na₂SO₄, and concentrated in vacuo. The residue was purified by silica gel chromatography (SilicaSep 40 g cartridge) using EtOAc and hexanes (0-100%) then MeOH and DCM (5-15%) to obtain an oil which was further purified by reverse phase chromatography (30 g C-18 cartridge) using acetonitrile and 0.1% aqueous formic acid to obtain 6,7-dimethyl-2-[2-(2-methyl-4-pyridyl)tetrahydropyran-4-yl]-4-methylsulfanyl-pteridine (255 mg, 0.655 mmol, 26%) as a solid. ESI-MS (m/z+): 382.10 [M+H]⁺, LC-RT: 2.136 min. ¹H NMR (400 MHz, CD₂Cl₂) δ ppm 8.41 (d, J=4.9 Hz, 1H), 7.23 (s, 1H), 7.14 (d, J=4.8 Hz, 1H), 4.56-4.49 (m, 1H), 4.37-4.28 (m, 1H), 3.85-3.77 (m, 1H), 3.48-3.38 (m, 1H), 2.74 (s, 3H), 2.72 (s, 3H), 2.66 (s, 3H), 2.52 (s, 3H), 2.43-2.36 (m, 1H), 2.17-2.09 (m, 2H), 1.95-1.84 (m, 1H).

Step 3: In a flame-dried 50 mL microwave vial 6,7-dimethyl-2-[2-(2-methyl-4-pyridyl)tetrahydropyran-4-yl]-4-methylsulfanyl-pteridine (122 mg, 0.320 mmol), Pd(OAc)₂ (1.8 mg, 0.0080 mmol), SPhos (6.6 mg, 0.016 mmol) and THF (1 mL) were added. The reaction mixture was degassed for 5 min under N₂ and chloro-(4-chloro-2,3-difluoro-phenyl)zinc chloride solution (0.089 M in THF) (5.3 mL, 0.4797 mmol) was added dropwise at 25° C. over 30 min. The mixture was stirred at 25° C. for 2 h. The reaction was quenched by addition of sat. NaHCO₃ (20 mL) and the reaction mixture was extracted with DCM (50 mL). The aqueous layer was extracted with (2×50 mL). The combined organic layer was dried over Na₂SO₄ and the solvent was removed in vacuo. The crude material was purified by flash chromatography (Isco RediSep® column 40 g) using EtOAc and hexanes (0-100%) then with MeOH and DCM (10-20%) to obtain solid (34 mg), which was further purified by prep HPLC (Gemini® 5 um NX-C18 110 A, 100×30 mm) using MeOH and aqueous ammonium bicarbonate to obtain a mixture of cis isomers 4-(4-chloro-3,5-difluoro-phenyl)-6,7-dimethyl-2-[rac-(2R,4S)-2-(2-methyl-4-pyridyl) tetrahydropyran-4-yl]pteridine (14 mg, 0.0277 mmol, 9%) as one peak and a mixture of trans isomers 4-(4-chloro-3,5-difluoro-phenyl)-6,7-dimethyl-2-[rac-(2R,4R)-2-(2-methyl-4-ridyl)tetrahydropyran-4-yl]pteridine (4.5 mg, 0.00907 mmol, 3%) as another peak. Cis isomers: ESI-MS (m/z+): 482.2 [M+H]⁺, LC-RT: 1.598 min. ¹H NMR (400 MHz, CD₂Cl₂) δ ppm 8.41 (s, 2H), 8.39 (s, 1H), 7.23 (s, 1H), 7.14 (d, J=4.0 Hz, 1H), 4.56 (dd, J=11.3, 1.1 Hz, 1H), 4.39-4.32 (m, 1H), 3.90-3.79 (m, 1H), 3.64-3.51 (m, 1H), 2.81 (s, 3H), 2.79 (s, 3H), 2.52 (s, 3H), 2.48-2.40 (m, 1H), 2.24-2.13 (m, 2H), 2.01-1.88 (m, 1H). ¹⁹F NMR (376 MHz, CD₂Cl₂) δ ppm −113.77 (s), −113.80 (s). trans isomers: ESI-MS (m/z+): 482.2 [M+H]⁺, LC-RT: 1.560 min. ¹H NMR (400 MHz, CD₂Cl₂) δ ppm 8.42 (d, J=5.0 Hz, 1H), 8.35 (d, J=8.2 Hz, 2H), 7.23 (s, 1H), 7.14 (d, J=4.9 Hz, 1H), 4.69-4.57 (m, 2H), 4.40-4.33 (m, 1H), 3.99-3.89 (m, 1H), 2.83 (s, 3H), 2.82 (s, 3H), 2.52 (s, 3H), 2.34-2.24 (m, 1H), 2.23-2.16 (m, 1H), 2.12-2.01 (m, 1H), 2.01-1.93 (m, 1H). ¹⁹F NMR (376 MHz, CD₂Cl₂) δ ppm −113.54 (s), −113.56 (s).

### Method 8

### Example 87: 7-((2R,4S)-2-(1-cyclopropyl-1H-pyrazol-4-yl)tetrahydro-2H-pyran-4-yl)-2,3-dimethyl-5-(3-(trifluoromethyl)bicyclo[1.1.1]pentan-1-yl)pyrido[3,4-b]pyrazine

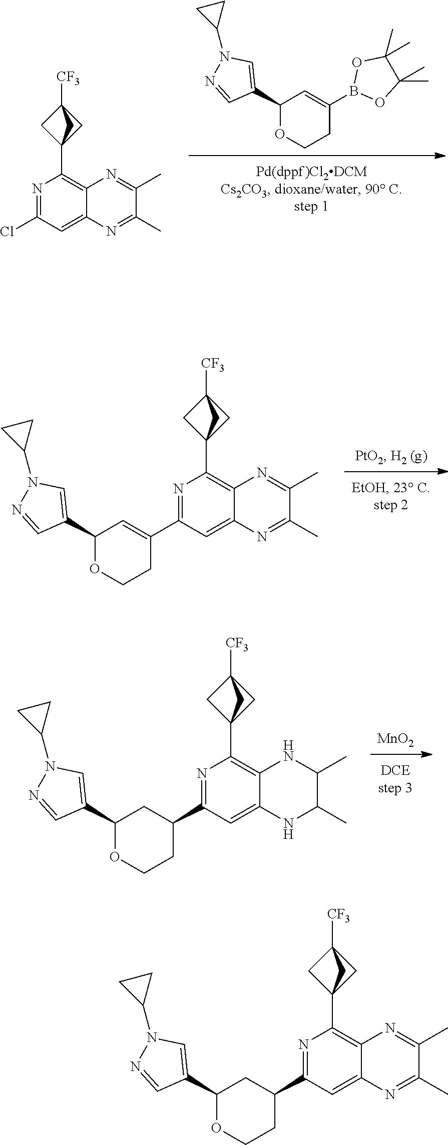

Step 1: To a solution of 7-chloro-2,3-dimethyl-5-[3-(trifluoromethyl)-1-bicyclo[1.1.1]pentanyl]pyrido[3,4-b]pyrazine (490 mg, 1.50 mmol, Intermediate 114) and 1-cyclopropyl-4-[(6R)-4-(4,4,5,5-tetramethyl-1,3,2-dioxaborolan-2-yl)-3,6-dihydro-2H-pyran-6-yl]pyrazole (520 mg, 1.64 mmol) in 1,4-dioxane (10 mL) was added cesium carbonate (1461 mg, 4.49 mmol), water (1 mL) and Pd(dppf)Cl₂ (109 mg, 0.150 mmol). The mixture was then stirred at 90° C. overnight. After completion, the mixture was cooled to r.t., diluted with EtOAc. The organic layer was then washed with water then brine and dried over MgSO₄, filtered through a plug of silica, and concentrated in vacuo. The residue was then purified by flash chromatography using a DCM/EtOAc gradient (20%-100%) to affords the desired material (560 mg, 75%) as a light-yellow foam. ¹H NMR (400 MHz, Chloroform-d): δ_{H} 7.69 (1H, s), 7.53 (1H, s), 7.50 (1H, s), 7.18 (1H, s), 5.42 (1H, d, J=2.9 Hz), 4.09-4.16 (1H, m), 3.93 (1H, m), 3.54-3.60 (1H, m), 2.74 (4H, s), 2.73 (3H, s), 2.67 (1H, m), 2.62 (6H, s), 1.10-1.13 (2H, m), 0.97-1.03 (2H, m).

Step 2: To a flask under argon atmosphere containing 2,3-dimethyl-7-[(6R)-6-(1-cyclopropylpyrazol-4-yl)-3,6-dihydro-2H-pyran-4-yl]-5-[3-(trifluoromethyl)-1-bicyclo[1.1.1]pentanyl]pyrido[3,4-b]pyrazine (1.00 eq, 254 mg, 0.528 mmol) in ethanol (8 mL) was added PtO₂ (0.710 eq, 85 mg, 0.374 mmol). The system was purged with hydrogen and stirred overnight under 1 atm of H₂. When the reaction was judged complete by LCMS and ¹H NMR, the mixture was diluted with EtOAc and filtered through celite and evaporated. The crude material was used in the next step without further purification.

Step 3: To a flask under argon atmosphere containing 2,3-dimethyl-7-[(2R,4S)-2-(1-cyclopropylpyrazol-4-yl)tetrahydropyran-4-yl]-5-[3-(trifluoromethyl)-1-bicyclo[1.1.1]pentanyl]-1,2,3,4-tetrahydropyrido[3,4-b]pyrazine (1.00 eq, 254 mg, 0.521 mmol) in DCE (5 mL) was added MnO₂ (20.1 eq, 900 mg, 10.5 mmol). The reaction was then stirred overnight at 50° C. After completion, the mixture was cooled down to r.t., diluted with EtOAc and filtered through a plug of silica and the solvent was evaporated in vacuo. The residue was purified by column chromatography using a 35%-100% DCM/EtOAc gradient to afford the desired material as a 11:1 diastereomeric mixture. Further purification by reverse phase chromatography using a Gemini® 5 um NX-C18 110 Å, 100×30 mm column and a 55%-75% methanol/water (10 mm ammonium formate) gradient gave the desired material (113 mg, 45%) as a white solid after lyophilization. ¹H NMR (400 MHz, Chloroform-d): δ ppm 7.54 (1H, s), 7.48 (2H, s), 4.55 (1H, d, J=11.2 Hz), 4.25 (1H, d, J=11.4 Hz), 3.84-3.78 (1H, m), 3.59-3.53 (1H, m), 3.22 (1H, m), 2.74 (3H, s), 2.73 (3H, s), 2.61 (6H, s), 2.30 (1H, d, J=13.1 Hz), 2.02-1.95 (3H, m), 1.10 (2H, m), 1.04-0.97 (2H, m).

### Method 9

### Example 89: 4-(4-chloro-2,3-difluorophenyl)-7-methyl-2-(2-(2-methylpyridin-4-yl)tetrahydro-2H-pyran-4-yl)pteridine

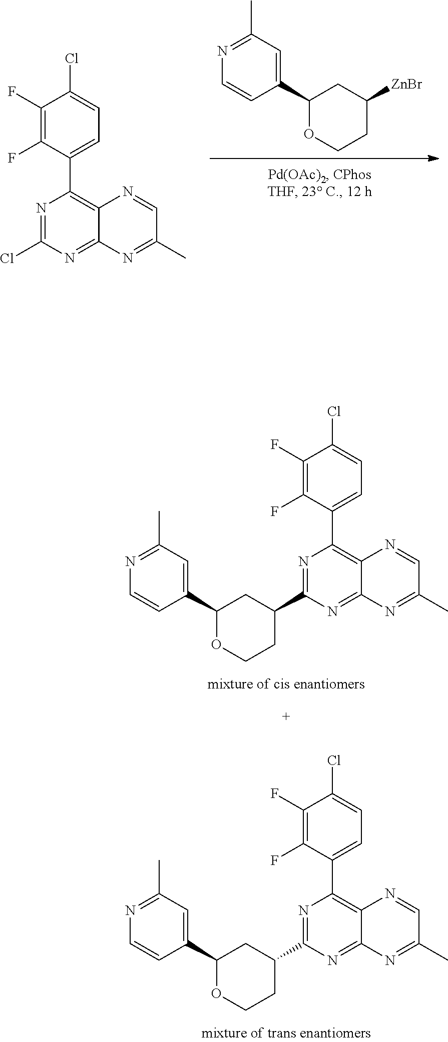

In a flame-dried 50 mL microwave vial, 2-chloro-4-(4-chloro-2,3-difluoro-phenyl)-7-methyl-pteridine (100 mg, 0.306 mmol), palladium acetate (6.9 mg, 0.0306 mmol), C-Phos (0.200 eq, 27 mg, 0.0611 mmol) and THF (3.5 mL) were added. The reaction mixture was degassed for 5 min under N₂ and bromo-[2-(2-methyl-4-pyridyl)tetrahydropyran-4-yl]zinc bromide solution (0.17 M in THF) (1.8 mL, 0.3057 mmol) was added dropwise over 30 min. The mixture was stirred at 22° C. for 2 h. The reaction was quenched by addition of sat. NaHCO₃ (20 mL) and the reaction mixture was extracted with DCM (50 mL). The aqueous layer was extracted with DCM (2×50 mL). The combined organic layer was dried over Na₂SO₄ and the solvent was removed in vacuo. The crude material was purified by flash chromatography (Isco RediSep® column 40 g) using EtOAc and hexanes (0-100%) then using MeOH and DCM (0-10%) to obtain a solid (100 mg) which was further purified by prep HPLC (Gemini® 5 um NX-C18 110 Å, 100×30 mm column) using MeOH and aqueous 10 mM ammonium formate to obtain 4-(4-chloro-2,3-difluoro-phenyl)-7-methyl-2-[rac-(2R,4S)-2-(2-methyl-4-pyridyl)tetrahydropyran-4-yl]pteridine as a mixture of cis diastereomers (32.3 mg, 22%) and 4-(4-chloro-2,3-difluoro-phenyl)-7-methyl-2-[rac-(2R,4R)-2-(2-methyl-4-pyridyl)tetrahydropyran-4-yl]pteridine as a mixture of trans diastereomers (2.8 mg, 2%). Cis isomers: ESI-MS (m/z+): 468.20 [M+H]⁺, LC-RT: 1.307 min. ¹H NMR (400 MHz, CD₂Cl₂) δ 8.81 (s, 1H), 8.41 (s, 1H), 7.50-7.45 (m, 1H), 7.43-7.37 (m, 1H), 7.23 (s, 1H), 7.13 (d, J=4.6 Hz, 1H), 4.55 (d, J=11.5 Hz, 1H), 4.34 (dd, J=10.6, 3.8 Hz, 1H), 3.84 (td, J=11.7, 3.2 Hz, 1H), 3.66-3.57 (m, 1H), 2.86 (s, 3H), 2.51 (s, 3H), 2.47-2.40 (m, 1H), 2.25-2.13 (m, 2H), 2.01-1.90 (m, 1H). ¹⁹F NMR (376 MHz, CD₂Cl₂) δ ppm −133.01 (s), −138.66 (s). Trans isomer: ¹H NMR (400 MHz, CD₂Cl₂) δ ppm 8.85 (s, 1H), 8.42 (d, J=5.5 Hz, 1H), 7.56-7.51 (m, 1H), 7.46-7.38 (m, 1H), 7.21 (s, 1H), 7.12 (d, J=4.7 Hz, 1H), 4.78 (dd, J=9.6, 2.4 Hz, 1H), 4.04-3.97 (m, 1H), 3.90 (td, J=11.3, 2.5 Hz, 1H), 3.76-3.71 (m, 1H), 2.89 (s, 3H), 2.52 (s, 3H), 2.52 (s, 2H), 2.30-2.24 (m, 1H), 2.22-2.16 (m, 1H).

### Method 10

### Example 97: 8-(4-chloro-2-fluorophenyl)-6-(2-(1-cyclopropyl-1H-pyrazol-4-yl)tetrahydro-2H-pyran-4-yl)-2,3-dimethylpyrido[2,3-b]pyrazine

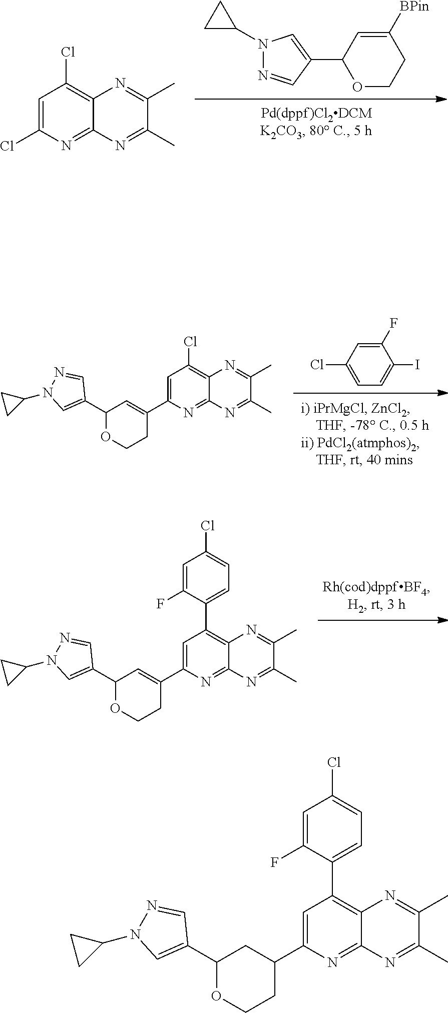

Step 1: To a solution of 6,8-dichloro-2,3-dimethylpyrido[2,3-b]pyrazine (1 g, 4.4 mmol) in dioxane (20 mL) and H₂O (4 mL) was added 1-cyclopropyl-4-(4-(4,4,5,5-tetramethyl-1,3,2-dioxaborolan-2-yl)-5,6-dihydro-2H-pyran-2-yl)-1H-pyrazole (1.4 g, 4.4 mmol) and K₂CO₃ (1.8 g, 13 mmol) and the reaction mixture was purged with nitrogen. Then Pd(dppf)Cl₂-DCM (0.29 g, 0.36 mmol) was added and the reaction mixture was heated at 80° C. for 5 h. The reaction mixture was then cooled to RT and monitored by LCMS. After completion, the aqueous layer was extracted with ethyl acetate (3×200 ml) and the combined organic layers were dried over anhydrous sodium sulfate and concentrated under reduced pressure to get the crude residue. The residue was purified via column chromatography on silica gel (PE:EA=1:1) to afford 8-chloro-6-(6-(1-cyclopropyl-1H-pyrazol-4-yl)-3,6-dihydro-2H-pyran-4-yl)-2,3-dimethylpyrido[2,3-b]pyrazine (1.3 g, 76%) as a purple solid. LCMS: (M+H)⁺=382.0;

Step 2: To a 250 mL round-bottomed flask was added 4-chloro-2-fluoro-1-iodobenzene (2.2 g, 8.6 mmol) in THF (40 mL). The mixture was cooled to −40° C. and iPrMgCl (4.7 mL, 9.5 mmol) (2 M solution in THF) was added dropwise and stirred for 30 min at −40° C., then the reaction mixture was cooled to −78° C. ZnCl₂ (4.3 mL, 8.6 mmol) (2 M solution in THF) was then added dropwise and the reaction mixture was allowed to warm to RT and 40 mL of THF was added and stirred for 10 min to give (4-chloro-2-fluorophenyl)zinc(II) iodide, which was used in the next reaction directly.

Into a 250-mL 3-necked round-bottom flask purged and maintained with N₂, was placed 8-chloro-6-(6-(1-cyclopropyl-1H-pyrazol-4-yl)-3,6-dihydro-2H-pyran-4-yl)-2,3-dimethylpyrido[2,3-b]pyrazine (1.1 g, 2.9 mmol) and PdCl₂(Atmphos)₂ (0.1 g, 0.14 mmol) in THF (10 mL). The reaction mixture was stirred and (4-chloro-2-fluorophenyl)zinc(II) iodide (2.2 g, 8.6 mmol) was added. The reaction mixture was stirred at room temp for 40 min and monitored by LCMS. After completion, the reaction mixture was quenched with H₂O (200 ml). The aqueous layer was extracted with EA (3×200 ml) and the combined organic layers were dried over anhydrous sodium sulfate, and then concentrated under reduced pressure to get the crude residue. The residue was purified via column chromatography on silica gel (PE:EA=1:1) to afford 8-(4-chloro-2-fluorophenyl)-6-(6-(1-cyclopropyl-1H-pyrazol-4-yl)-3,6-dihydro-2H-pyran-4-yl)-2,3-dimethylpyrido[2,3-b]pyrazine (900 mg, 64%) as a white solid. LCMS: (M+1)⁺=476.0.

Step 3: To a solution of 8-(4-chloro-2-fluorophenyl)-6-(6-(1-cyclopropyl-1H-pyrazol-4-yl)-3,6-dihydro-2H-pyran-4-yl)-2,3-dimethylpyrido[2,3-b]pyrazine (400 mg, 0.84 mmol) in THF (8 mL) was added Rh(cod)dppf·BF₄ (122 mg, 0.17 mmol) and the reaction mixture was purged with hydrogen for 3 h at room temp. The reaction was monitored by LCMS. After completion the reaction mixture was evaporated under reduced pressure to get the crude residue. The residue was purified by silica gel chromatography (PE:EA=1:2) to afford 8-(4-chloro-2-fluorophenyl)-6-(2-(1-cyclopropyl-1H-pyrazol-4-yl)tetrahydro-2H-pyran-4-yl)-2,3-dimethylpyrido[2,3-b]pyrazine (123 mg, 31%) as a white solid. LCMS: (M+H)⁺=478.0.

### Method 11

### Example 210: 7-((2R,4S)-2-(1-cyclopropyl-1H-pyrazol-4-yl)tetrahydro-2H-pyran-4-yl)-5-(2,4-difluorophenyl)-2,3-dimethylpyrido[3,4-b]pyrazine

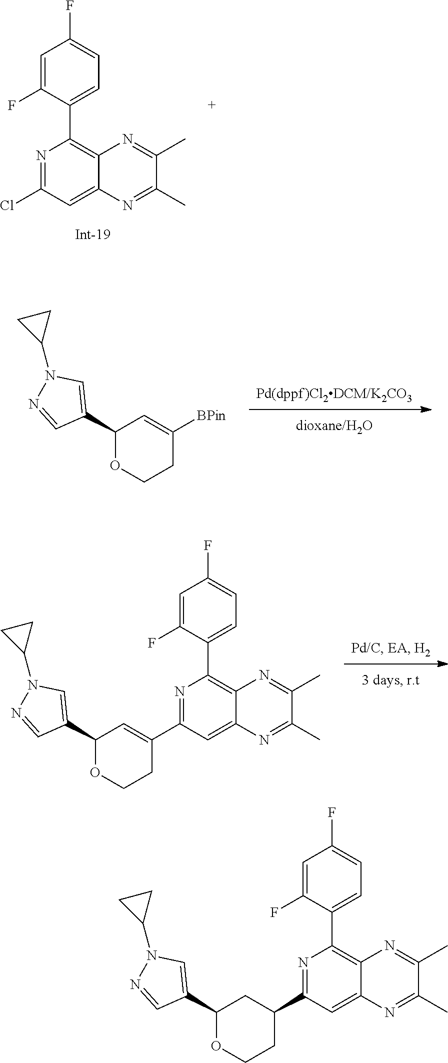

Step 1: To a mixture of 7-chloro-5-(2,4-difluorophenyl)-2,3-dimethylpyrido[3,4-b]pyrazine (583 mg, 1.635 mmol, 1.0 eq), (R)-1-cyclopropyl-4-(4-(4,4,5,5-tetramethyl-1,3,2-dioxaborolan-2-yl)-5,6-dihydro-2H-pyran-2-yl)-1H-pyrazole (371 mg, 1.962 mmol, 1.2 eq) and K₂CO₃ (678 mg, 4.905 mmol, 3.0 eq) in dioxane (10 mL) and H₂O (2 mL) was added Pd(dppf)Cl₂·DCM (107 mg, 0.131 mmol, 0.08 eq) under N₂ and the reaction mixture was purged with N₂ three times and stirred at 80° C. for 5 h to give a brown suspension. The reaction mixture was filtered through diatomite and washed with EtOAc (50 mL * 3), then extracted with EtOAc (150 mL*3). The combined organic phase was washed with brine (100 mL), dried over anhydrous Na₂SO₄, filtered and concentrated in vacuum to give crude product. The crude product was purified by column chromatography (SiO₂, PE/EA=15:1-5:1) to give the desired product of (R)-7-(6-(1-cyclopropyl-1H-pyrazol-4-yl)-3,6-dihydro-2H-pyran-4-yl)-5-(2,4-difluorophenyl)-2,3-dimethylpyrido[3,4-b]pyrazine (513 mg, 68%) as yellow solid. LCMS: (M+H)⁺=460.1; purity=99% (UV 254 nm); Retention time=2.044 min.

Step 2: To a solution of 5-(2,4-difluorophenyl)-2,3-dimethyl-7-[rac-(6R)-6-(1-cyclopropylpyrazol-4-yl)-3,6-dihydro-2H-pyran-4-yl]pyrido[3,4-b]pyrazine (1.0 eq, 35 mg, 0.0762 mmol) in ethyl acetate (4 mL) was added palladium on carbon 10% (15 mg). The reaction was filtered through a diatomite pad. The filtrate was concentrated under reduced pressure. The crude product was purified by column chromatography (DCM/MeOH 20:1 to 10:1), then purified further by prep-HPLC (A: water (NH₄HCO₃), B: acetonitrile) to afford 7-[(2R,4S)-2-(1-cyclopropylpyrazol-4-yl)tetrahydropyran-4-yl]-5-(2,4-difluorophenyl)-2,3-dimethyl-pyrido[3,4-b]pyrazine (9.2 mg, 0.0199 mmol, 26.17%) as a white solid.

### Method 12

### Examples 227 and 228: 5-(4-chloro-2-fluoro-phenyl)-7-[(2S,4R)-2-(1-cyclopropylpyrazol-4-yl)tetrahydropyran-4-yl]-2,3-dimethyl-quinoxaline and 5-(4-chloro-2-fluoro-phenyl)-7-[(2R,4S)-2-(1-cyclopropylpyrazol-4-yl)tetrahydropyran-4-yl]-2,3-dimethyl-quinoxaline

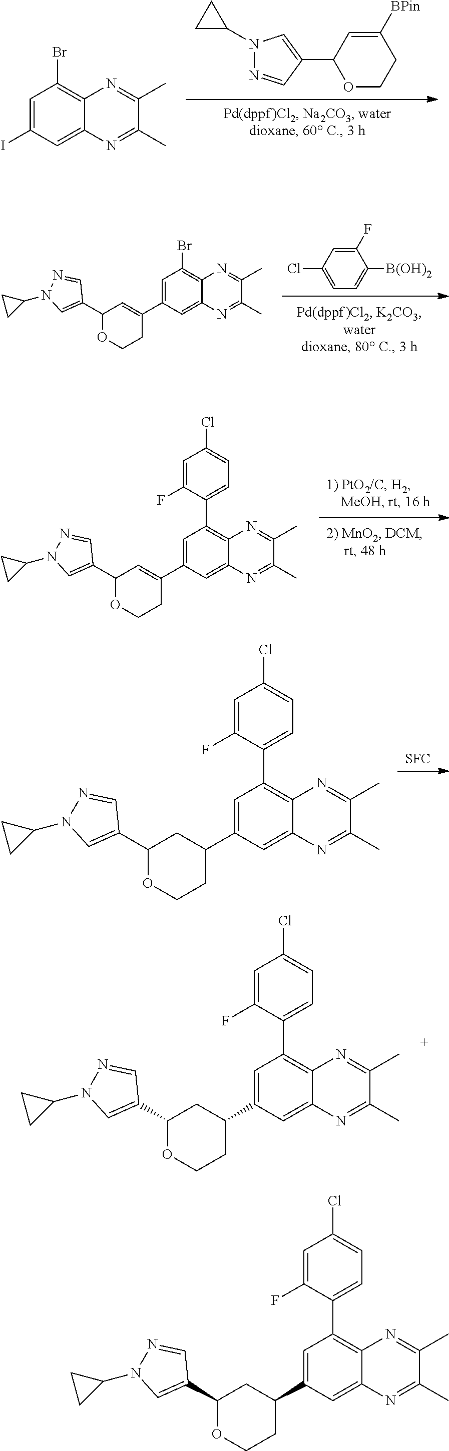

Step 1: A mixture of 5-bromo-7-iodo-2,3-dimethyl-quinoxaline (1.00 eq, 460 mg, 1.27 mmol), 1-cyclopropyl-4-[4-(4,4,5,5-tetramethyl-1,3,2-dioxaborolan-2-yl)-3,6-dihydro-2H-pyran-6-yl]pyrazole (1.00 eq, 401 mg, 1.27 mmol), Pd(dppf)Cl₂ (0.1000 eq, 93 mg, 0.127 mmol) and sodium carbonate (2.00 eq, 269 mg, 2.53 mmol) in 1,4-dioxane (10 mL) and water (1 mL) under argon was stirred at 60° C. for 4 h. The reaction was filtered and concentrated under reduced pressure. The residue was purified by column chromatography (70% EtOAc in PE) to give 5-bromo-7-[6-(1-cyclopropylpyrazol-4-yl)-3,6-dihydro-2H-pyran-4-yl]-2,3-dimethyl-quinoxaline (360 mg, 0.694 mmol, 54.77% yield) as a brown solid. LCMS: Rt: 2.269 min; [M+H]⁺=486.1.

Step 2: To a solution of 1,4-dioxane (8 mL)/water (1 mL) was added 5-bromo-7-[(6R)-6-(1-cyclopropylpyrazol-4-yl)-3,6-dihydro-2H-pyran-4-yl]-2,3-dimethyl-quinoxaline (1.00 eq, 110 mg, 0.259 mmol), (4-chloro-2-fluoro-phenyl)boronic acid (1.00 eq, 20 mg, 0.117 mmol) and KOAc (1.50 eq, 57 mg, 0.176 mmol) at room temperature. Pd(dppf)Cl₂ (0.100 eq, 8.6 mg, 0.0118 mmol) was then added to the solution under N₂ and stirred at 100° C. for 16 h. The mixture was washed with water (30 mL) and extracted with ethyl acetate (30 mL*3). The combined organic layer was dried over anhydrous sodium sulphate, concentrated under reduced pressure to get the crude residue. The residue was purified via column chromatography on silica gel (PE/EtOAc=1/1) to afford 5-(4-chloro-2-fluoro-phenyl)-7-[(6R)-6-(1-cyclopropylpyrazol-4-yl)-3,6-dihydro-2H-pyran-4-yl]-2,3-dimethyl-quinoxaline (76 mg, 0.154 mmol, 67.20% yield). LCMS: Rt: 2.215 min; [M+H]⁺=476.7; 96.67% purity at 254 nm.

Step 3: PtO₂ (1.00 eq, 36 mg, 0.160 mmol) was added to a solution of 5-(4-chloro-2-fluoro-phenyl)-7-[6-(1-cyclopropylpyrazol-4-yl)-3,6-dihydro-2H-pyran-4-yl]-2,3-dimethyl-quinoxaline (1.00 eq, 76 mg, 0.160 mmol) in THF (5 mL) under H₂ atmosphere. The mixture was stirred at 25° C. for 2 hours. The mixture was filtered and concentrated. DCM (5 mL) and MnO₂ (10.0 eq, 139 mg, 1.60 mmol) were added and the mixture was stirred at 25° C. for 16 hours. The mixture was washed with water (30 mL) and extracted with ethyl acetate (30 mL*3). The organic layer was dried with anhydrous sodium sulfate, filtered and concentrated under reduced pressure. The residue was purified by prep-HPLC to obtain a mixture of racemics (50 mg, 100% purity, 65.65% yield) as a white solid. LC-MS: Rt: 2.164 min; [M+H]⁺=477.0; 100% purity at 254 nm.

The racemic mixture was separated by SFC to obtain 5-(4-chloro-2-fluoro-phenyl)-7-[(2S,4R)-2-(1-cyclopropylpyrazol-4-yl)tetrahydropyran-4-yl]-2,3-dimethyl-quinoxaline (7.1 mg, 0.0149 mmol, 9.32% yield) and 5-(4-chloro-2-fluoro-phenyl)-7-[(2R,4S)-2-(1-cyclopropylpyrazol-4-yl)tetrahydropyran-4-yl]-2,3-dimethyl-quinoxaline (7.1 mg, 0.0149 mmol, 9.32% yield) as white solids.

### Method 13

### Example 157 and 158: 2-[(2R,4S,6R)-2-(1-cyclopropylpyrazol-4-yl)-6-methyl-tetrahydropyran-4-yl]-6,7-dimethyl-4-(2,4,5-trifluorophenyl)pteridine and 2-[(2R,4R,6R)-2-(1-cyclopropylpyrazol-4-yl)-6-methyl-tetrahydropyran-4-yl]-6,7-dimethyl-4-(2,4,5-trifluorophenyl)pteridine

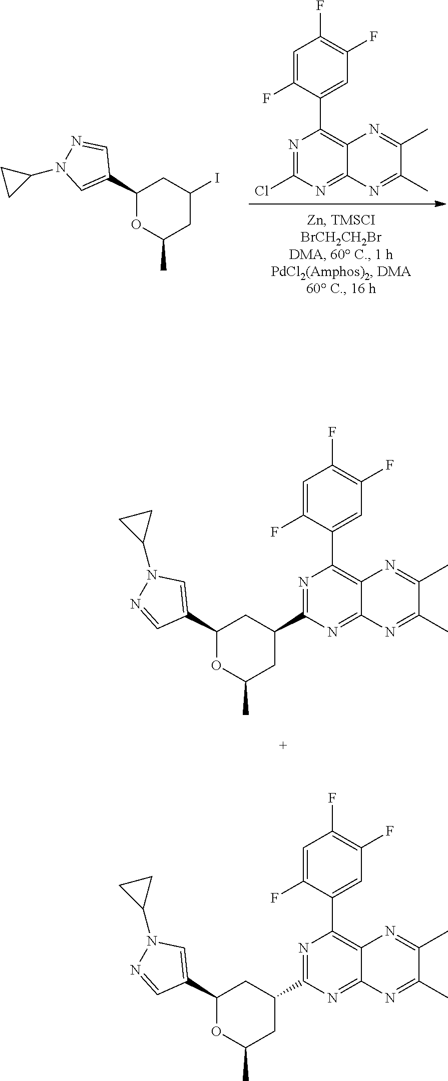 [SMILES: C[C@@H]1CC(I)C[C@H](c2cnn(C3CC3)c2)O1 ; Cc1nc2nc(Cl)nc(-c3cc(F)c(F)cc3F)c2nc1C ; Cc1nc2nc([C@H]3C[C@@H](C)O[C@@H](c4cnn(C5CC5)c4)C3)nc(-c3cc(F)c(F)cc3F)c2nc1C ; Cc1nc2nc([C@@H]3C[C@@H](C)O[C@@H](c4cnn(C5CC5)c4)C3)nc(-c3cc(F)c(F)cc3F)c2nc1C]

To a mixture of Zinc dust (3.00 eq, 392 mg, 6.00 mmol) in DMA (4 mL) was added BrCH₂CH₂Br (1.00 eq, 0.10 mL, 2.00 mmol) under argon protection and the mixture was stirred at r.t for 10 min. TMSCl (1.00 eq, 0.10 mL, 2.00 mmol) was added dropwise and the mixture was stirred at 60° C. for 30 min. A solution of 1-cyclopropyl-4-[(2R,6R)-4-iodo-6-methyl-tetrahydropyran-2-yl]pyrazole (1.00 eq, 664 mg, 2.00 mmol) in DMA (2 mL) was added to the mixture and the mixture was stirred at 60° C. for 1 h. 1 mL of suspension was added to a mixture of 2-chloro-6,7-dimethyl-4-(2,4,5-trifluorophenyl)pteridine (0.251 eq, 163 mg, 0.502 mmol) and PdCl₂(Amphos) (0.0353 eq, 50 mg, 0.0706 mmol) under argon protection. The mixture was stirred at 60° C. for 16 h. The mixture was washed with water (30 mL) and extracted with ethyl acetate (30 mL*3). The organic phase was concentrated and chromatographed on silica gel (DCM/MeOH=25/1) to give the crude (50 mg) as a red solid. It was purified by prep-HPLC to afford a mixture. The mixture of isomers was separated by SFC to afford 2-[(2R,4R,6R)-2-(1-cyclopropylpyrazol-4-yl)-6-methyl-tetrahydropyran-4-yl]-6,7-dimethyl-4-(2,4,5-trifluorophenyl)pteridine (3.6 mg, 0.00728 mmol, 1.80% yield) and 2-[(2R,4S,6R)-2-(1-cyclopropylpyrazol-4-yl)-6-methyl-tetrahydropyran-4-yl]-6,7-dimethyl-4-(2,4,5-trifluorophenyl)pteridine (21 mg, 0.0421 mmol, 10.4% yield) as a yellow oil.

### Method 14

### Example 166: 2-(2-(3-cyclopropyl-1H-pyrazol-5-yl)tetrahydro-2H-pyran-4-yl)-4-(2,4-difluorophenyl)-6,7-dimethylpteridine

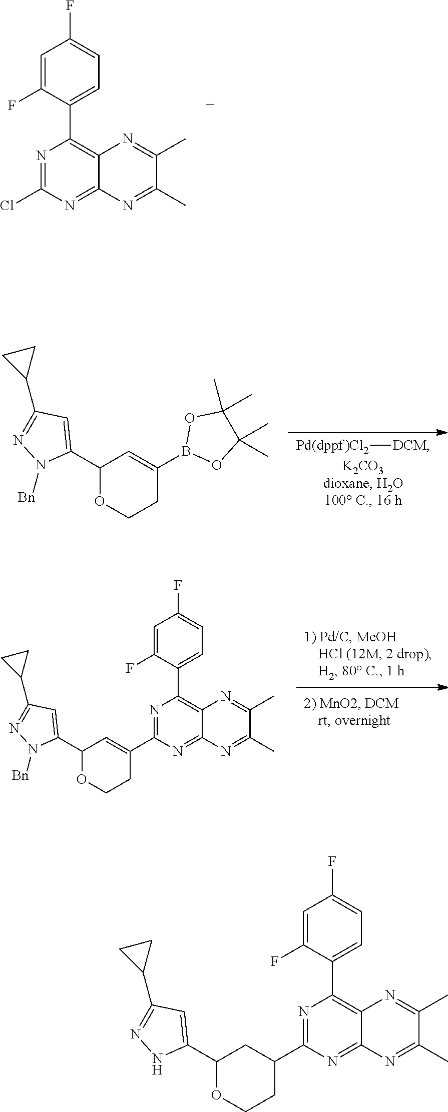

Step 1: To a solution of 1-benzyl-3-cyclopropyl-5-[4-(4,4,5,5-tetramethyl-1,3,2-dioxaborolan-2-yl)-3,6-dihydro-2H-pyran-6-yl]pyrazole (1.00 eq, 250 mg, 0.615 mmol), 2-chloro-4-(2,4-difluorophenyl)-6,7-dimethyl-pteridine (2.00 eq, 287 mg, 0.935 mmol) and potassium carbonate (3.00 eq, 194 mg, 1.40 mmol) in 1,4-dioxane (5 mL) was added 1,1′-Bis(diphenylphosphino)ferrocene-palladium(II)dichloride dichloromethane complex (0.100 eq, 38 mg, 0.0468 mmol) under nitrogen. The reaction was stirred at 100° C. overnight. The reaction was concentrated to dryness and the residue was taken up in EtOAc (200 mL) and the organics washed with 2*50 mL water, followed by 50 mL of saturated brine solution. The organics were then separated and dried with MgSO₄ before concentration to dryness. The crude residue was then purified by flash column chromatography eluting with 40% EtOAc in petroleum ether. The desired fractions were concentrated to dryness in vacuo to afford 2-[6-(2-benzyl-5-cyclopropyl-pyrazol-3-yl)-3,6-dihydro-2H-pyran-4-yl]-4-(2,4-difluorophenyl)-6,7-dimethyl-pteridine (100 mg, 0.182 mmol, 29.60% yield) as a yellow solid. LC-MS: Rt: 2.30 min; [M+H]⁺=551.3.

Step 2: To a solution of 2-[6-(2-benzyl-5-cyclopropyl-pyrazol-3-yl)-3,6-dihydro-2H-pyran-4-yl]-4-(2,4-difluorophenyl)-6,7-dimethyl-pteridine (1.00 eq, 90 mg, 0.163 mmol) in methanol (20 mL) was added Pt/C (1.00 eq, 200 mg, 0.163 mmol) and hydrochloric acid (20 mg). The reaction was stirred at 80° C. for 1 h. The reaction mixture was filtered and concentrated to afford a crude material. The crude material was dissolved in dichloromethane and then NH₃-MeOH (0.5 mL, 7N) was added. The mixture was concentrated to get a crude material. The crude material was dissolved in dichloromethane (20 mL) and manganese dioxide (10.0 eq, 142 mg, 1.63 mmol) was added. The reaction was stirred at 20° C. overnight. The reaction mixture was concentrated and filtered to get the crude product. The crude product was purified by Prep-HPLC to afford 2-[2-(3-cyclopropyl-1H-pyrazol-5-yl)tetrahydropyran-4-yl]-4-(2,4-difluorophenyl)-6,7-dimethyl-pteridine (1.6 mg, 0.00346 mmol, 2.12% yield) as a light yellow solid.

### Method 15

### Example 168: 4-(2,4-difluorophenyl)-6,7-dimethyl-2-((2R,6R)-2-methyl-6-(1H-pyrazol-4-yl tetrahydro-2H-pyran-4-yl pteridine

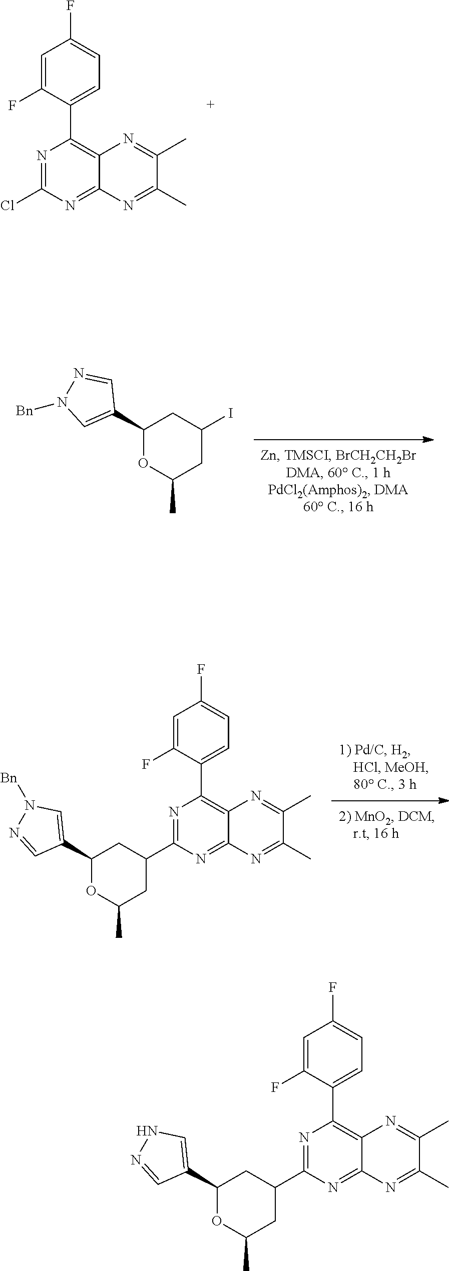 [SMILES: Cc1nc2nc(Cl)nc(-c3ccc(F)cc3F)c2nc1C ; C[C@@H]1CC(I)C[C@H](c2cnn(Cc3ccccc3)c2)O1 ; Cc1nc2nc(C3C[C@@H](C)O[C@@H](c4cnn(Cc5ccccc5)c4)C3)nc(-c3ccc(F)cc3F)c2nc1C ; Cc1nc2nc(C3C[C@@H](C)O[C@@H](c4cn[nH]c4)C3)nc(-c3ccc(F)cc3F)c2nc1C]

Step 1: To a mixture of Zinc dust (6.13 eq, 392 mg, 6.00 mmol) in DMA (4 mL) was added BrCH₂CH₂Br (2.04 eq, 368 mg, 2.00 mmol) in a glove box and the mixture was stirred at r.t for 10 min. TMSCl (2.04 eq, 217 mg, 2.00 mmol) was added dropwise and the mixture was stirred at 60° C. for 30 min. A solution of 1-benzyl-4-[(2R,6R)-4-iodo-6-methyl-tetrahydropyran-2-yl]pyrazole (2.04 eq, 764 mg, 2.00 mmol) in DMA (2 mL) was added to the mixture and the mixture was stirred at 60° C. for 1 h. 1 mL of the suspension was added to a mixture of 2-chloro-4-(2,4-difluorophenyl)-6,7-dimethyl-pteridine (1.00 eq, 300 mg, 0.978 mmol) and PdCl₂(Amphos) (0.0722 eq, 50 mg, 0.0706 mmol) under argon protection. The mixture was stirred at 60° C. for 16 h. The mixture was extracted with EtOAc (30 mL*2) and washed with water (10 mL*2). The organic layer was dried and concentrated. The residue was purified with prep-TLC (UV254, Silica, DCM/MeOH=20/1) to give 2-((2R,6R)-2-(1-benzyl-1H-pyrazol-4-yl)-6-methyltetrahydro-2H-pyran-4-yl)-4-(2,4-difluorophenyl)-6,7-dimethylpteridine as a yellow solid. (100 mg, 19% yield). LC-MS: Rt: 2.003 min; [M+H]⁺=527; 96.90% purity at 214 nm.

Step 2: To a solution of 2-[(2R,6R)-2-(1-benzylpyrazol-4-yl)-6-methyl-tetrahydropyran-4-yl]-4-(2,4-difluorophenyl)-6,7-dimethyl-pteridine (1.00 eq, 80 mg, 0.152 mmol) in methanol (30 mL) was added Pd/C (6.21 eq, 100 mg, 0.943 mmol) and HCl (3 drops). The reaction mixture was stirred at 80° C. under H₂ for 3 h. The mixture was filtered, and the filtrate was poured into NH₃ in MeOH (2 mL, 7 N) and purified by prep-TLC (Silic, UV 254, DCM/MeOH=20/1) to give the over-reduced intermediate as a yellow solid. (50 mg, 89% yield). Then the crude intermediate was dissolved in DCM (20 mL), and MnO₂ (50.0 eq, 660 mg, 7.60 mmol) was added. The mixture was stirred at room temperature overnight. The mixture was then filtered and the filtrate was concentrated, then purified by prep-HPLC (NH₄HCO₃) to afford 4-(2,4-difluorophenyl)-6,7-dimethyl-2-[2-(1H-pyrazol-4-yl)tetrahydropyran-4-yl]pteridine (7.1 mg, 0.0163 mmol, 10.71% yield) as a white solid.

### Method 16

### Example 201: 5-[(2R,4S)-4-[4-(2,4-difluorophenyl)-6,7-dimethyl-pteridin-2-yl]tetrahydropyran-2-yl]-1-methyl-pyridin-2-one

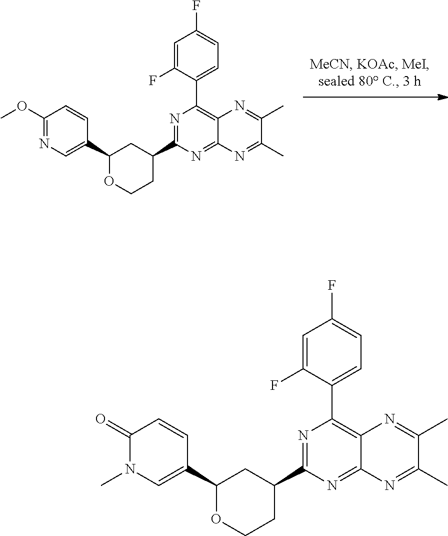

Step 1: A solution of 4-(2,4-difluorophenyl)-2-[(2R,4S)-2-(6-methoxy-3-pyridyl)tetrahydropyran-4-yl]-6,7-dimethyl-pteridine (1.00 eq, 50 mg, 0.108 mmol) and KOAc (2.00 eq, 21 mg, 0.216 mmol) in MeCN (5 mL) was placed under N₂, then it was Mel (1.00 eq, 15 mg, 0.108 mmol) was added and the mixture was stirred at 80° C. for 3 hours. The mixture was purified with prep-HPLC to give 5-[(2R,4S)-4-[4-(2,4-difluorophenyl)-6,7-dimethyl-pteridin-2-yl]tetrahydropyran-2-yl]-1-methyl-pyridin-2-one (15 mg, 0.0324 mmol, 30.00% yield) as green solid.

### Method 17

### Example 203: 3-((2R,4S)-4-(4-(2,4-difluorophenyl)-6,7-dimethylpteridin-2-yl)tetrahydro-2H-pyran-2-yl)-1-methylpyridin-2(1H)-one

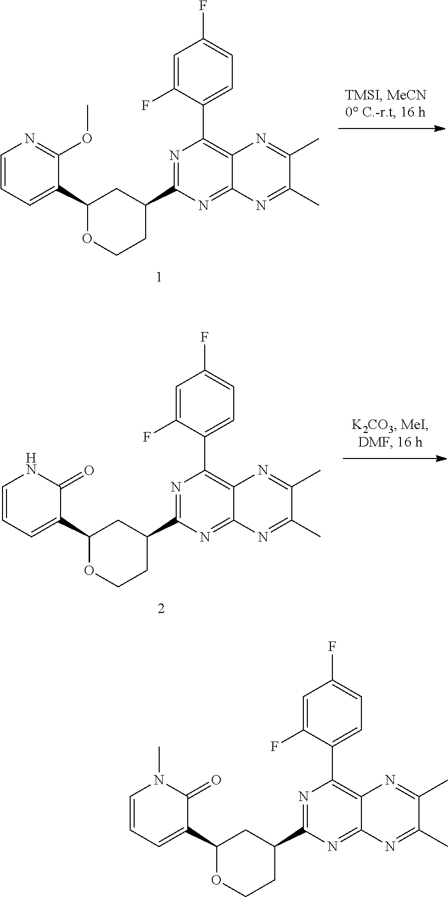 [SMILES: COc1ncccc1[C@H]1C[C@@H](c2nc(-c3ccc(F)cc3F)c3nc(C)c(C)nc3n2)CCO1 ; Cc1nc2nc([C@H]3CCO[C@@H](c4ccc[nH]c4=O)C3)nc(-c3ccc(F)cc3F)c2nc1C ; Cc1nc2nc([C@H]3CCO[C@@H](c4cccn(C)c4=O)C3)nc(-c3ccc(F)cc3F)c2nc1C]

Step 1: To a solution of 4-(2,4-difluorophenyl)-2-[(2R,4S)-2-(2-methoxy-3-pyridyl)tetrahydropyran-4-yl]-6,7-dimethyl-pteridine (1.00 eq, 45 mg, 0.0971 mmol) in MeCN (5 mL) was added TMSI (1.00 eq, 19 mg, 0.0971 mmol) in MeCN (2.5 mL). The mixture was stirred at 0° C. under N₂ protection for 16 hours. After 16 h, LC-MS showed DP/SM=1/2. The mixture was extracted with ethyl acetate (30 mL*2) and washed with water (30 ml*2). The organic layer was concentrated to give crude 3-[(2R,4S)-4-[4-(2,4-difluorophenyl)-6,7-dimethyl-pteridin-2-yl]tetrahydropyran-2-yl]-1H-pyridin-2-one (50 mg, 0.0200 mmol, 20.62% yield) as a yellow solid which was used in the following step without purification. LC-MS: Rt: 1.39 min, m/z: 450.1 [M+H]⁺. 18% purity at 254 nm.

Step 2: A solution of 3-[(2R,4S)-4-[4-(2,4-difluorophenyl)-6,7-dimethyl-pteridin-2-yl]tetrahydropyran-2-yl]-1H-pyridin-2-one (1.00 eq, 50 mg, 0.0200 mmol), K₂CO₃ (5.00 eq, 14 mg, 0.100 mmol) and Mel (5.00 eq, 14 mg, 0.100 mmol) in DMF (3 mL) was stirred at 25° C. for 16 hours. The mixture was extracted with ethyl acetate (30 mL*2) and washed with water (30 ml*2) and brine (50 mL). The organic layer was concentrated and purified with prep-HPLC to give 3-[(2R,4S)-4-[4-(2,4-difluorophenyl)-6,7-dimethyl-pteridin-2-yl]tetrahydropyran-2-yl]-1-methyl-pyridin-2-one (5.0 mg, 0.0108 mmol, 53.87% yield) as a white solid.

TABLE B
Exemplary Compounds
The compounds disclosed below in Table B were made by a method of thepresent disclosure
or a similar method. The appropriate reagents, starting materials and conditions necessary for
synthesizing the compounds of Table B would be apparent to aperson of ordinary skill in the art.
Compounds designated with “(+/−)” were isolated as a mixture of diastereomers sharing the same relative
stereochemistry (ie. cis or trans). Compounds designated with “(rac)” were isolated as a mixture of all
possible stereoisomers of the shown compound. Compounds lacking either designation were isolated
with the specific stereochemistry shown, such that the specific stereoisomer shown made up at least 90%
of the isolatedproduct.

| | | | Method used to |
|---|---|---|---|
| Ex # | Structure | Name | synthesize |
| 1 | 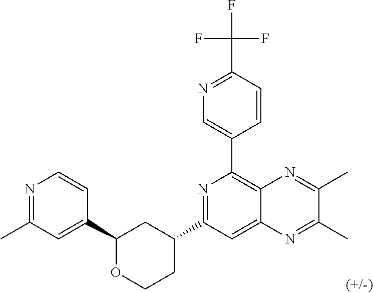 | (2,3-dimethyl-7-(2-(2- methylpyridin-4-yl)tetrahydro- 2H-pyran-4-yl)-5-(6- (trifluoromethyl)pyridin-3- yl)pyrido[3,4-b]pyrazine | Method 1 |
| 2 | 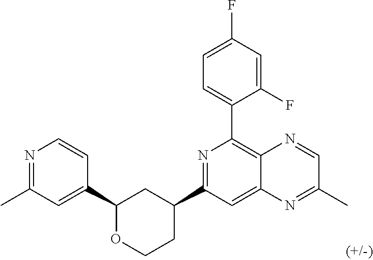 | 5-(2,4-difluorophenyl)-2-methyl- 7-(2-(2-methylpyridin-4- yl)tetrahydro-2H-pyran-4- yl)pyrido[3,4-b]pyrazine | Method 1 |
| 3 | 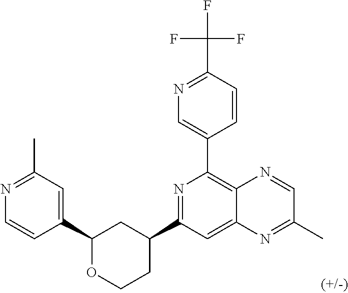 [SMILES: Cc1cc([C@H]2C[C@@H](c3cc4nc(C)cnc4c(-c4ccc(C(F)(F)F)nc4)n3)CCO2)ccn1] | 2-methyl-7-(2-(2-methylpyridin- 4-yl)tetrahydro-2H-pyran-4-yl)- 5-(6-(trifluoromethyl)pyridin-3- yl)pyrido[3,4-b]pyrazine | Method 1 |
| 4 | 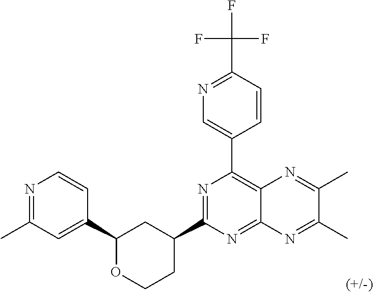 | 6,7-dimethyl-2-(2-(2- methylpyridin-4-yl)tetrahydro- 2H-pyran-4-yl)-4-(6- (trifluoromethyl)pyridin-3- yl)pteridine | Method 1 |
| 5 | 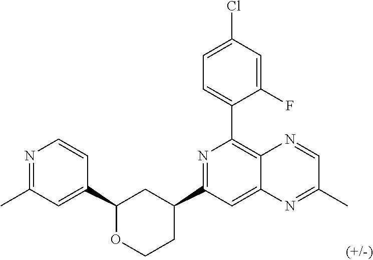 | 5-(4-chloro-2-fluorophenyl)-2- methyl-7-(2-(2-methylpyridin-4- yl)tetrahydro-2H-pyran-4- yl)pyrido[3,4-b]pyrazine | Method 1 |
| 6 | 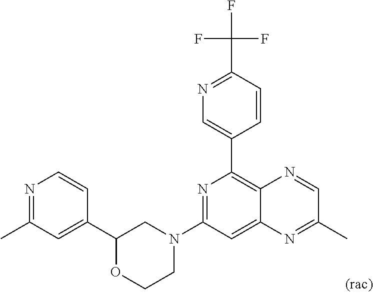 | 4-(2-methyl-5-(6- (trifluoromethyl)pyridin-3- yl)pyrido[3,4-b]pyrazin-7-yl)-2- (2-methylpyridin-4- yl)morpholine | Method 3 |
| 7 | 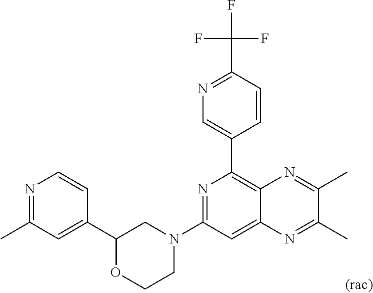 | 4-(2,3-dimethyl-5-(6- (trifluoromethyl)pyridin-3- yl)pyrido[3,4-b]pyrazin-7-yl)-2- (2-methylpyridin-4- yl)morpholine | Method 3 |
| 8 | 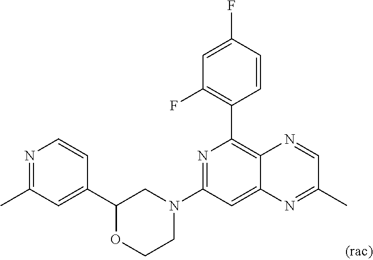 | 4-(5-(2,4-difluorophenyl)-2- methylpyrido[3,4-b]pyrazin-7- yl)-2-(2-methylpyridin-4- yl)morpholine | Method 3 |
| 9 | 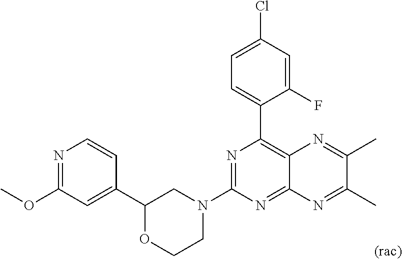 | 4-(4-(4-chloro-2-fluorophenyl)- 6,7-dimethylpteridin-2-yl)-2-(2- methoxypyridin-4-yl)morpholine | Method 6 |
| 10 | 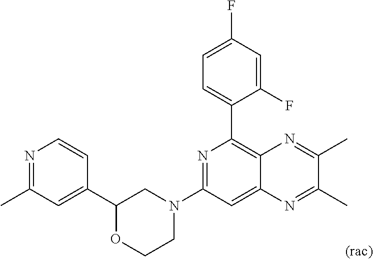 | 4-(5-(2,4-difluorophenyl)-2,3- dimethylpyrido[3,4-b]pyrazin-7- yl)-2-(2-methylpyridin-4- yl)morpholine | Method 3 |

| | | | |
|---|---|---|---|
| 11 | 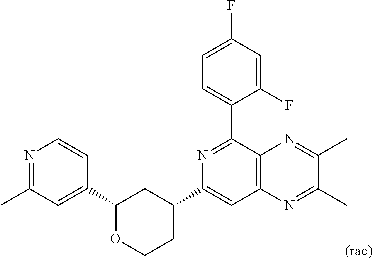 | 5-(2,4-difluorophenyl)-2,3- dimethyl-7-[(2S,4R)-2-(2- methyl-4- pyridyl)tetrahydropyran-4- yl]pyrido[3,4-b]pyrazine | Method 1 |

| | | | |
|---|---|---|---|
| 12 | 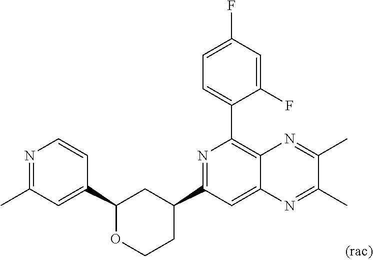 | 5-(2,4-difluorophenyl)-2,3- dimethyl-7-[(2R,4S)-2-(2- methyl-4- pyridyl)tetrahydropyran-4- yl]pyrido[3,4-b]pyrazine | Method 1 |

| | | | |
|---|---|---|---|
| 13 | 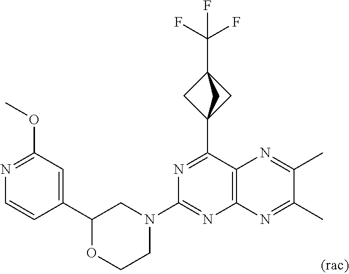 [SMILES: COc1cc(C2CN(c3nc4nc(C)c(C)nc4c([C@]45C[C@](C(F)(F)F)(C4)C5)n3)CCO2)ccn1] | 4-(6,7-dimethyl-4-(3- (trifluoromethyl)bicyclo[1.1.1] pentan-1-yl)pteridin-2-yl)-2-(2- methoxypyridin-4-yl)morpholine | Method 6 |

| | | | |
|---|---|---|---|
| 14 | 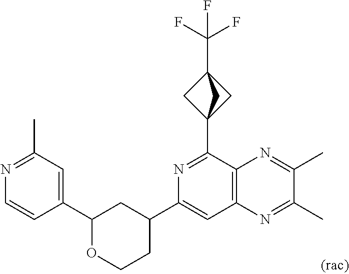 [SMILES: Cc1cc(C2CC(c3cc4nc(C)c(C)nc4c([C@]45C[C@](C(F)(F)F)(C4)C5)n3)CCO2)ccn1] | 2,3-dimethyl-7-(2-(2- methylpyridin-4-yl)tetrahydro- 2H-pyran-4-yl)-5-(3- (trifluoromethyl)bicyclo[1.1.1] pentan-1-yl)pyrido[3,4-b]pyrazine | Method 1 |

| | | | |
|---|---|---|---|
| 15 | 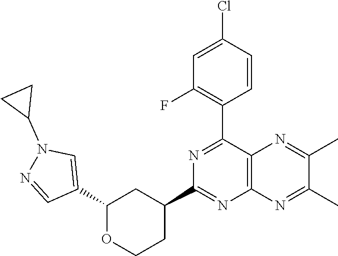 [SMILES: Cc1nc2nc([C@H]3CCO[C@H](c4cnn(C5CC5)c4)C3)nc(-c3ccc(Cl)cc3F)c2nc1C] | 4-(4-chloro-2-fluorophenyl)-2- ((2S,4S)-2-(1-cyclopropyl-1H- pyrazol-4-yl)tetrahydro-2H- pyran-4-yl)-6,7- dimethylpteridine | Method 2 |

| | | | |
|---|---|---|---|
| 16 | 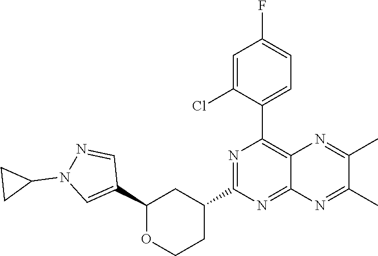 [SMILES: Cc1nc2nc([C@@H]3CCO[C@@H](c4cnn(C5CC5)c4)C3)nc(-c3ccc(F)cc3Cl)c2nc1C] | 4-(2-chloro-4-fluorophenyl)-2- ((2R,4R)-2-(1-cyclopropyl-1H- pyrazol-4-yl)tetrahydro-2H- pyran-4-yl)-6,7- dimethylpteridine | Method 2 |

| | | | |
|---|---|---|---|
| 17 | 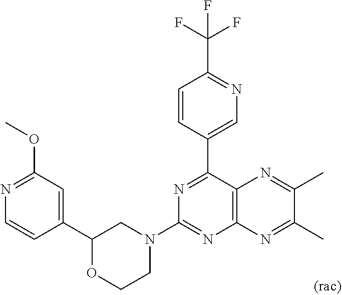 [SMILES: COc1cc(C2CN(c3nc(-c4ccc(C(F)(F)F)nc4)c4nc(C)c(C)nc4n3)CCO2)ccn1] | 4-(6,7-dimethyl-4-(6- (trifluoromethyl)pyridin-3- yl)pteridin-2-yl)-2-(2- methoxypyridin-4-yl)morpholine | Method 1 |

| | | | |
|---|---|---|---|
| 18 | 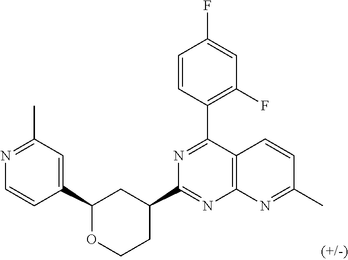 [SMILES: Cc1cc([C@H]2C[C@@H](c3nc(-c4ccc(F)cc4F)c4ccc(C)nc4n3)CCO2)ccn1] | 4-(2,4-difluorophenyl)-7-methyl- 2-(2-(2-methylpyridin-4- yl)tetrahydro-2H-pyran-4- yl)pyrido[2,3-d]pyrimidine | Method 1 |

| | | | |
|---|---|---|---|
| 19 | 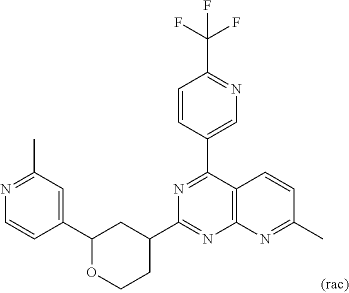 [SMILES: Cc1cc(C2CC(c3nc(-c4ccc(C(F)(F)F)nc4)c4ccc(C)nc4n3)CCO2)ccn1] | 7-methyl-2-(2-(2-methylpyridin- 4-yl)tetrahydro-2H-pyran-4-yl)- 4-(6-(trifluoromethyl)pyridin-3- yl)pyrido[2,3-d]pyrimidine | Method 1 |

| | | | |
|---|---|---|---|
| 20 | 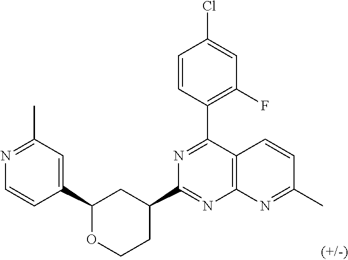 [SMILES: Cc1cc([C@H]2C[C@@H](c3nc(-c4ccc(Cl)cc4F)c4ccc(C)nc4n3)CCO2)ccn1] | 4-(4-chloro-2-fluorophenyl)-7- methyl-2-(2-(2-methylpyridin-4- yl)tetrahydro-2H-pyran-4- yl)pyrido[2,3-d]pyrimidine | Method 1 |

| | | | |
|---|---|---|---|
| 21 | 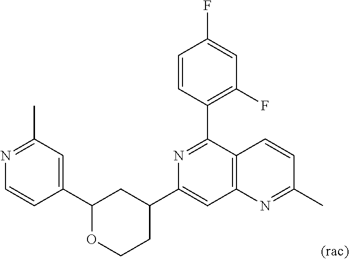 [SMILES: Cc1cc(C2CC(c3cc4nc(C)ccc4c(-c4ccc(F)cc4F)n3)CCO2)ccn1] | 5-(2,4-difluorophenyl)-2-methyl- 7-(2-(2-methylpyridin-4- yl)tetrahydro-2H-pyran-4-yl)- 1,6-naphthyridine | Method 1 |

| | | | |
|---|---|---|---|
| 22 | 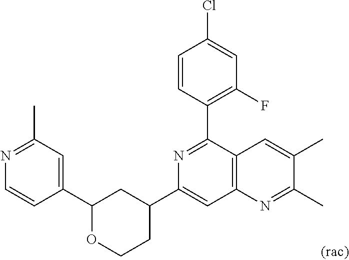 [SMILES: Cc1cc(C2CC(c3cc4nc(C)c(C)cc4c(-c4ccc(Cl)cc4F)n3)CCO2)ccn1] | 5-(4-chloro-2-fluorophenyl)-2,3- dimethyl-7-(2-(2-methylpyridin- 4-yl)tetrahydro-2H-pyran-4-yl)- 1,6-naphthyridine | Method 1 |

| | | | |
|---|---|---|---|
| 23 | 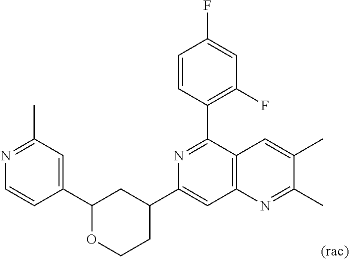 [SMILES: Cc1cc(C2CC(c3cc4nc(C)c(C)cc4c(-c4ccc(F)cc4F)n3)CCO2)ccn1] | 5-(2,4-difluorophenyl)-2,3- dimethyl-7-(2-(2-methylpyridin- 4-yl)tetrahydro-2H-pyran-4-yl)- 1,6-naphthyridine | Method 1 |

| | | | |
|---|---|---|---|
| 24 | 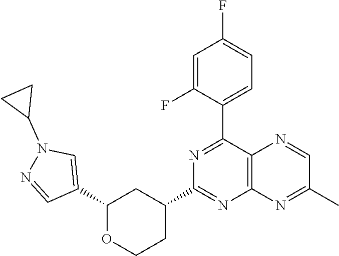 [SMILES: Cc1cnc2c(-c3ccc(F)cc3F)nc([C@@H]3CCO[C@H](c4cnn(C5CC5)c4)C3)nc2n1] | 2-[(2S,4R)-2-(1- cyclopropylpyrazol-4- yl)tetrahydropyran-4-yl]-4-(2,4- difluorophenyl)-7-methyl- pteridine | Method 2 |

| | | | |
|---|---|---|---|
| 25 | 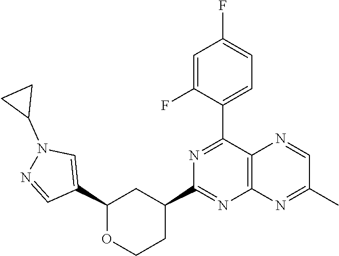 [SMILES: Cc1cnc2c(-c3ccc(F)cc3F)nc([C@H]3CCO[C@@H](c4cnn(C5CC5)c4)C3)nc2n1] | 2-[(2R,4S)-2-(1- cyclopropylpyrazol-4- yl)tetrahydropyran-4-yl]-4-(2,4- difluorophenyl)-7-methyl- pteridine | Method 2 |

| | | | |
|---|---|---|---|
| 26 | 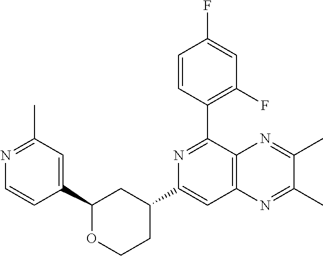 [SMILES: Cc1cc([C@H]2C[C@H](c3cc4nc(C)c(C)nc4c(-c4ccc(F)cc4F)n3)CCO2)ccn1] | 5-(2,4-difluorophenyl)-2,3- dimethyl-7-((2R,4R)-2-(2- methylpyridin-4-yl)tetrahydro- 2H-pyran-4-yl)pyrido[3,4- b]pyrazine | Method 3 |

| | | | |
|---|---|---|---|
| 27 | 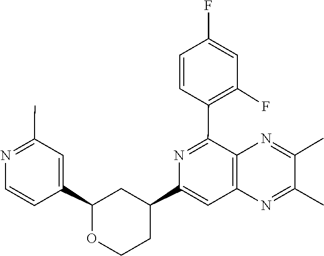 [SMILES: Cc1cc([C@H]2C[C@@H](c3cc4nc(C)c(C)nc4c(-c4ccc(F)cc4F)n3)CCO2)ccn1] | 5-(2,4-difluorophenyl)-2,3- dimethyl-7-((2R,4S)-2-(2- methylpyridin-4-yl)tetrahydro- 2H-pyran-4-yl)pyrido[3,4- b]pyrazine | Method 3 |

| | | | |
|---|---|---|---|
| 28 | 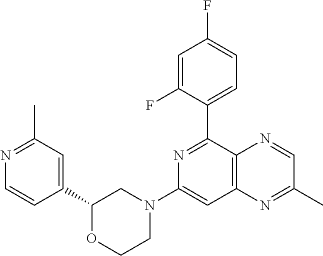 [SMILES: Cc1cc([C@@H]2CN(c3cc4nc(C)cnc4c(-c4ccc(F)cc4F)n3)CCO2)ccn1] | (R)-4-(5-(2,4-difluorophenyl)-2- methylpyrido[3,4-b]pyrazin-7- yl)-2-(2-methylpyridin-4- yl)morpholine | Method 3 |

| | | | |
|---|---|---|---|
| 29 | 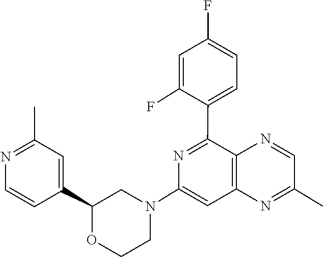 [SMILES: Cc1cc([C@H]2CN(c3cc4nc(C)cnc4c(-c4ccc(F)cc4F)n3)CCO2)ccn1] | (S)-4-(5-(2,4-difluorophenyl)-2- methylpyrido[3,4-b]pyrazin-7- yl)-2-(2-methylpyridin-4- yl)morpholine | Method 3 |

| | | | |
|---|---|---|---|
| 30 | 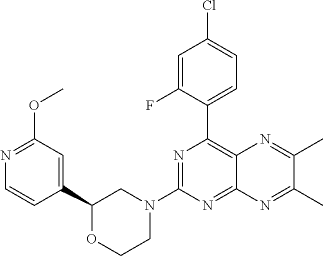 [SMILES: COc1cc([C@H]2CN(c3nc(-c4ccc(Cl)cc4F)c4nc(C)c(C)nc4n3)CCO2)ccn1] | (S)-4-(4-(4-chloro-2- fluorophenyl)-6,7- dimethylpteridin-2-yl)-2-(2- methoxypyridin-4-yl)morpholine | Method 6 |

| | | | |
|---|---|---|---|
| 31 | 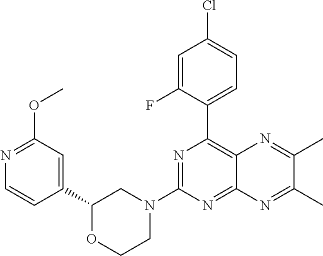 [SMILES: COc1cc([C@@H]2CN(c3nc(-c4ccc(Cl)cc4F)c4nc(C)c(C)nc4n3)CCO2)ccn1] | (R)-4-(4-(4-chloro-2- fluorophenyl)-6,7- dimethylpteridin-2-yl)-2-(2- methoxypyridin-4-yl)morpholine | Method 6 |

| | | | |
|---|---|---|---|
| 32 | 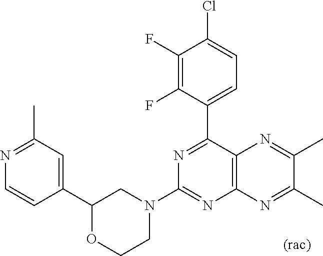 [SMILES: Cc1cc(C2CN(c3nc(-c4ccc(Cl)c(F)c4F)c4nc(C)c(C)nc4n3)CCO2)ccn1] | 4-(4-(4-chloro-2,3- difluorophenyl)-6,7- dimethylpteridin-2-yl)-2-(2- methylpyridin-4-yl)morpholine | Method 6 |

| | | | |
|---|---|---|---|
| 33 | 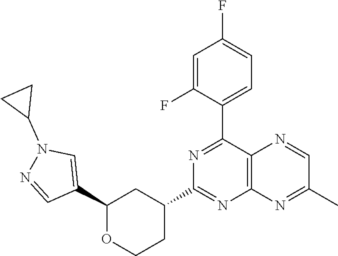 [SMILES: Cc1cnc2c(-c3ccc(F)cc3F)nc([C@@H]3CCO[C@@H](c4cnn(C5CC5)c4)C3)nc2n1] | 2-((2R,4R)-2-(1-cyclopropyl- 1H-pyrazol-4-yl)tetrahydro-2H- pyran-4-yl)-4-(2,4- difluorophenyl)-7- methylpteridine | Method 2 |

| | | | |
|---|---|---|---|
| 34 | 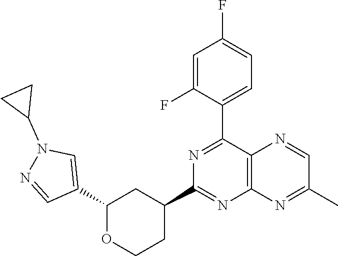 [SMILES: Cc1cnc2c(-c3ccc(F)cc3F)nc([C@H]3CCO[C@H](c4cnn(C5CC5)c4)C3)nc2n1] | 2-((2S,4S)-2-(1-cyclopropyl-1H- pyrazol-4-yl)tetrahydro-2H- pyran-4-yl)-4-(2,4- difluorophenyl)-7- methylpteridine | Method 2 |

| | | | |
|---|---|---|---|
| 35 | 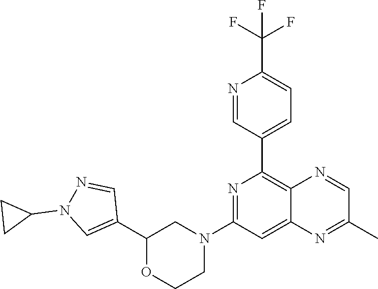 [SMILES: Cc1cnc2c(-c3ccc(C(F)(F)F)nc3)nc(N3CCOC(c4cnn(C5CC5)c4)C3)cc2n1] | 2-(1-cyclopropyl-1H-pyrazol-4- yl)-4-(2-methyl-5-(6- (trifluoromethyl)pyridin-3- yl)pyrido[3,4-b]pyrazin-7- yl)morpholine | Method 3 |

| | | | |
|---|---|---|---|
| 36 | 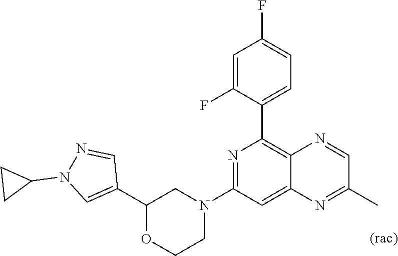 [SMILES: Cc1cnc2c(-c3ccc(F)cc3F)nc(N3CCOC(c4cnn(C5CC5)c4)C3)cc2n1] | 2-(1-cyclopropyl-1H-pyrazol-4- yl)-4-(5-(2,4-difluorophenyl)-2- methylpyrido[3,4-b]pyrazin-7- yl)morpholine | Method 3 |

| | | | |
|---|---|---|---|
| 37 | 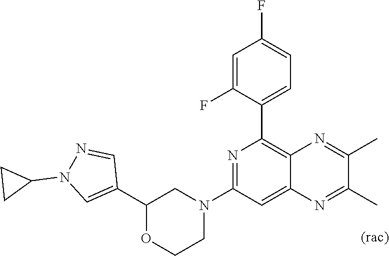 [SMILES: Cc1nc2cc(N3CCOC(c4cnn(C5CC5)c4)C3)nc(-c3ccc(F)cc3F)c2nc1C] | 2-(1-cyclopropyl-1H-pyrazol-4- yl)-4-(5-(2,4-difluorophenyl)- 2,3-dimethylpyrido[3,4- b]pyrazin-7-yl)morpholine | Method 3 |

| | | | |
|---|---|---|---|
| 38 | 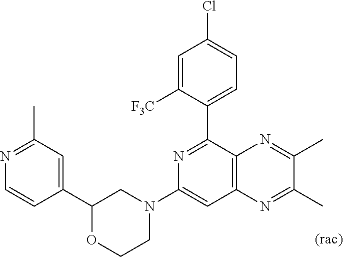 [SMILES: Cc1cc(C2CN(c3cc4nc(C)c(C)nc4c(-c4ccc(Cl)cc4C(F)(F)F)n3)CCO2)ccn1] | 4-(4-(4-chloro-2- (trifluoromethyl)phenyl)-6,7- dimethylpteridin-2-yl)-2-(2- methylpyridin-4-yl)morpholine | Method 6 |

| | | | |
|---|---|---|---|
| 39 | 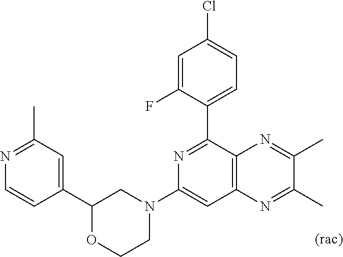 [SMILES: Cc1cc(C2CN(c3cc4nc(C)c(C)nc4c(-c4ccc(Cl)cc4F)n3)CCO2)ccn1] | 4-(5-(4-chloro-2-fluorophenyl)- 2,3-dimethyl-1,6-naphthyridin-7- yl)-2-(2-methylpyridin-4- yl)morpholine | Method 4 |

| | | | |
|---|---|---|---|
| 40 | 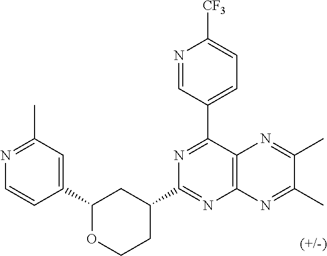 [SMILES: Cc1cc([C@@H]2C[C@H](c3nc(-c4ccc(C(F)(F)F)nc4)c4nc(C)c(C)nc4n3)CCO2)ccn1] | 6,7-dimethyl-2-(2-(2- methylpyridin-4-yl)tetrahydro- 2H-pyran-4-yl)-4-(6- (trifluoromethyl)pyridin-3- yl)pteridine | Method 1 |

| | | | |
|---|---|---|---|
| 41 | 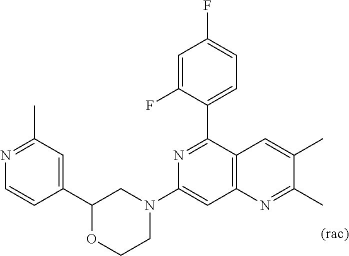 [SMILES: Cc1cc(C2CN(c3cc4nc(C)c(C)cc4c(-c4ccc(F)cc4F)n3)CCO2)ccn1] | 4-(5-(2,4-difluorophenyl)-2,3- dimethyl-1,6-naphthyridin-7-yl)- 2-(2-methylpyridin-4- yl)morpholine | Method 4 |

| | | | |
|---|---|---|---|
| 42 | 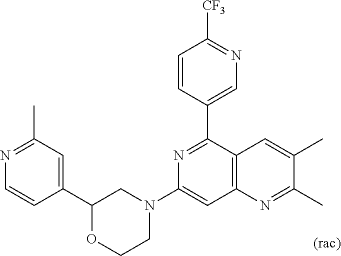 [SMILES: Cc1cc(C2CN(c3cc4nc(C)c(C)cc4c(-c4ccc(C(F)(F)F)nc4)n3)CCO2)ccn1] | 4-(2,3-dimethyl-5-(6- (trifluoromethyl)pyridin-3-yl)- 1,6-naphthyridin-7-yl)-2-(2- methylpyridin-4-yl)morpholine | Method 4 |

| | | | |
|---|---|---|---|
| 43 | 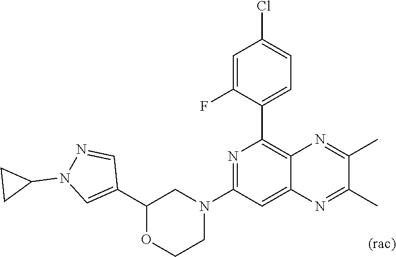 [SMILES: Cc1nc2cc(N3CCOC(c4cnn(C5CC5)c4)C3)nc(-c3ccc(Cl)cc3F)c2nc1C] | 4-(5-(4-chloro-2-fluorophenyl)- 2,3-dimethylpyrido[3,4- b]pyrazin-7-yl)-2-(1- cyclopropyl-1H-pyrazol-4- yl)morpholine | Method 3 |

| | | | |
|---|---|---|---|
| 44 | 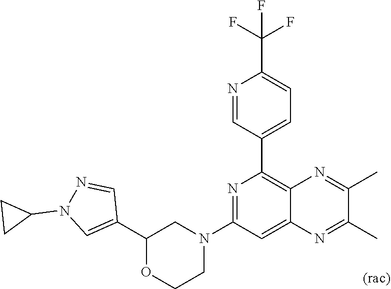 [SMILES: Cc1nc2cc(N3CCOC(c4cnn(C5CC5)c4)C3)nc(-c3ccc(C(F)(F)F)nc3)c2nc1C] | 2-(1-cyclopropyl-1H-pyrazol-4- yl)-4-(2,3-dimethyl-5-(6- (trifluoromethyl)pyridin-3- yl)pyrido[3,4-b]pyrazin-7- yl)morpholine | Method 3 |

| | | | |
|---|---|---|---|
| 45 | 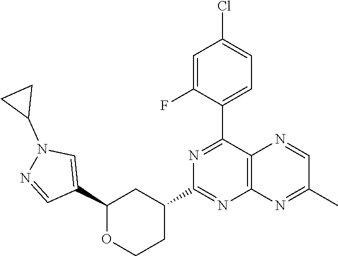 [SMILES: Cc1cnc2c(-c3ccc(Cl)cc3F)nc([C@@H]3CCO[C@@H](c4cnn(C5CC5)c4)C3)nc2n1] | 4-(4-chloro-2-fluorophenyl)-2- ((2R,4R)-2-(1-cyclopropyl-1H- pyrazol-4-yl)tetrahydro-2H- pyran-4-yl)-7-methylpteridine | Method 2 |

| | | | |
|---|---|---|---|
| 46 | 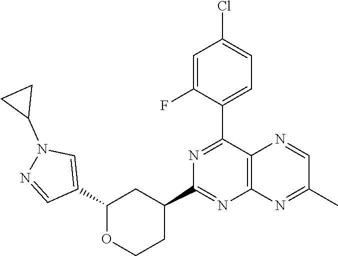 [SMILES: Cc1cnc2c(-c3ccc(Cl)cc3F)nc([C@H]3CCO[C@H](c4cnn(C5CC5)c4)C3)nc2n1] | 4-(4-chloro-2-fluorophenyl)-2- (2-(1-cyclopropyl-1H-pyrazol-4- yl)tetrahydro-2H-pyran-4-yl)-7- methylpteridine | Method 2 |

| | | | |
|---|---|---|---|
| 47 | 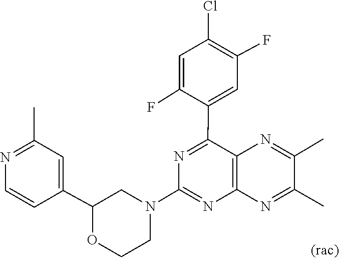 [SMILES: Cc1cc(C2CN(c3nc(-c4cc(F)c(Cl)cc4F)c4nc(C)c(C)nc4n3)CCO2)ccn1] | 4-(4-(4-chloro-2,5- difluorophenyl)-6,7- dimethylpteridin-2-yl)-2-(2- methylpyridin-4-yl)morpholine | Method 6 |

| | | | |
|---|---|---|---|
| 48 | 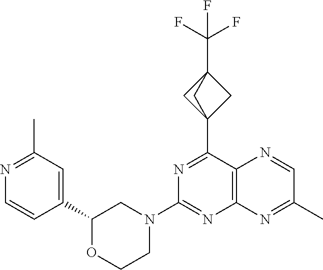 [SMILES: Cc1cc([C@@H]2CN(c3nc(C45CC(C(F)(F)F)(C4)C5)c4ncc(C)nc4n3)CCO2)ccn1] | (R)-4-(7-methyl-4-(3- (trifluoromethyl)bicyclo[1.1.1] pentan-1-yl)pteridin-2-yl)-2-(2- methylpyridin-4-yl)morpholine | Method 6 |

| | | | |
|---|---|---|---|
| 49 | 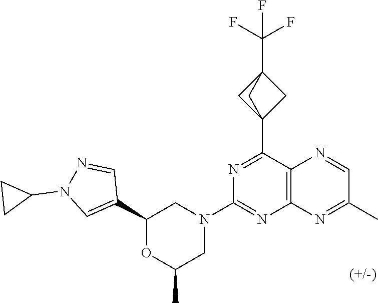 [SMILES: Cc1cnc2c(C34CC(C(F)(F)F)(C3)C4)nc(N3C[C@@H](C)O[C@@H](c4cnn(C5CC5)c4)C3)nc2n1] | 2-(1-cyclopropyl-1H-pyrazol-4- yl)-6-methyl-4-(7-methyl-4-(3- (trifluoromethyl)bicyclo[1.1.1] pentan-1-yl)pteridin-2- yl)morpholine | Method 6 |

| | | | |
|---|---|---|---|
| 50 | 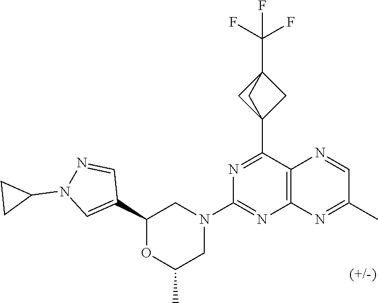 [SMILES: Cc1cnc2c(C34CC(C(F)(F)F)(C3)C4)nc(N3C[C@H](C)O[C@@H](c4cnn(C5CC5)c4)C3)nc2n1] | 2-(1-cyclopropyl-1H-pyrazol-4- yl)-6-methyl-4-(7-methyl-4-(3- (trifluoromethyl)bicyclo[1.1.1] pentan-1-yl)pteridin-2- yl)morpholine | Method 6 |

| | | | |
|---|---|---|---|
| 51 | 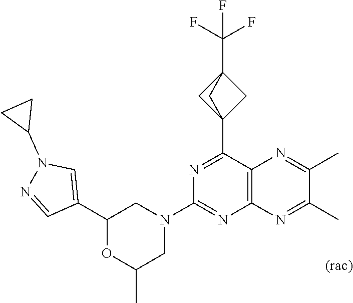 [SMILES: Cc1nc2nc(N3CC(C)OC(c4cnn(C5CC5)c4)C3)nc(C34CC(C(F)(F)F)(C3)C4)c2nc1C] | 2-(1-cyclopropyl-1H-pyrazol-4- yl)-4-(6,7-dimethyl-4-(3- (trifluoromethyl)bicyclo[1.1.1] pentan-1-yl)pteridin-2-yl)-6- methylmorpholine | Method 6 |

| | | | |
|---|---|---|---|
| 52 | 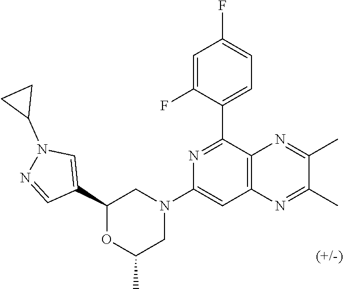 [SMILES: Cc1nc2cc(N3C[C@H](C)O[C@@H](c4cnn(C5CC5)c4)C3)nc(-c3ccc(F)cc3F)c2nc1C] | 2-(1-cyclopropyl-1H-pyrazol-4- yl)-4-(5-(2,4-difluorophenyl)- 2,3-dimethylpyrido[3,4- b]pyrazin-7-yl)-6- methylmorpholine | Method 5 |

| | | | |
|---|---|---|---|
| 53 | 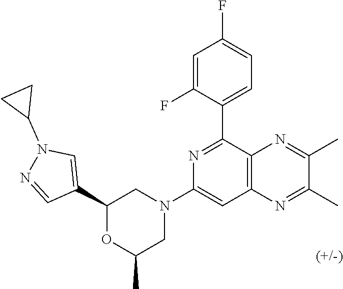 [SMILES: Cc1nc2cc(N3C[C@@H](C)O[C@@H](c4cnn(C5CC5)c4)C3)nc(-c3ccc(F)cc3F)c2nc1C] | 2-(1-cyclopropyl-1H-pyrazol-4- yl)-4-(5-(2,4-difluorophenyl)- 2,3-dimethylpyrido[3,4- b]pyrazin-7-yl)-6- methylmorpholine | Method 5 |

| | | | |
|---|---|---|---|
| 54 | 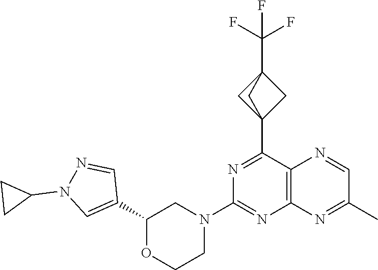 [SMILES: Cc1cnc2c(C34CC(C(F)(F)F)(C3)C4)nc(N3CCO[C@H](c4cnn(C5CC5)c4)C3)nc2n1] | (R)-2-(1-cyclopropyl-1H- pyrazol-4-yl)-4-(7-methyl-4-(3- (trifluoromethyl)bicyclo[1.1.1] pentan-1-yl)pteridin-2- yl)morpholine | Method 6 |

| | | | |
|---|---|---|---|
| 55 | 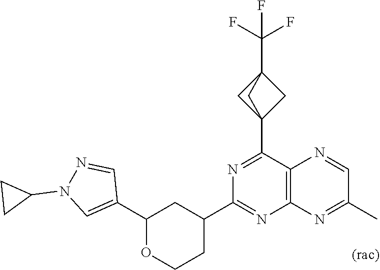 [SMILES: Cc1cnc2c(C34CC(C(F)(F)F)(C3)C4)nc(C3CCOC(c4cnn(C5CC5)c4)C3)nc2n1] | 2-(2-(1-cyclopropyl-1H-pyrazol- 4-yl)tetrahydro-2H-pyran-4-yl)- 7-methyl-4-(3- (trifluoromethyl)bicyclo[1.1.1] pentan-1-yl)pteridine | Method 2 |

| | | | |
|---|---|---|---|
| 56 | 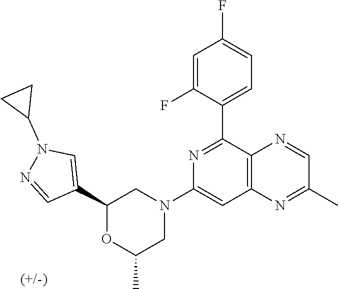 [SMILES: Cc1cnc2c(-c3ccc(F)cc3F)nc(N3C[C@H](C)O[C@@H](c4cnn(C5CC5)c4)C3)cc2n1] | 2-(1-cyclopropyl-1H-pyrazol-4- yl)-4-(5-(2,4-difluorophenyl)-2- methylpyrido[3,4-b]pyrazin-7- yl)-6-methylmorpholine | Method 5 |

| | | | |
|---|---|---|---|
| 57 | 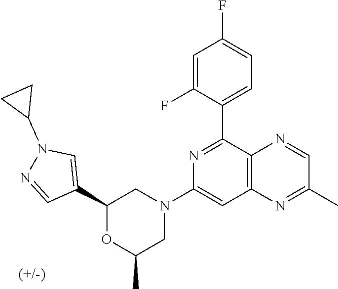 [SMILES: Cc1cnc2c(-c3ccc(F)cc3F)nc(N3C[C@@H](C)O[C@@H](c4cnn(C5CC5)c4)C3)cc2n1] | 2-(1-cyclopropyl-1H-pyrazol-4- yl)-4-(5-(2,4-difluorophenyl)-2- methylpyrido[3,4-b]pyrazin-7- yl)-6-methylmorpholine | Method 5 |

| | | | |
|---|---|---|---|
| 58 | 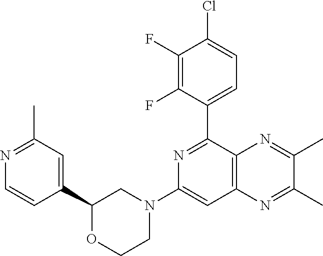 [SMILES: Cc1cc([C@H]2CN(c3cc4nc(C)c(C)nc4c(-c4ccc(Cl)c(F)c4F)n3)CCO2)ccn1] | (S)-4-(4-(4-chloro-2,3- difluorophenyl)-6,7- dimethylpteridin-2-yl)-2-(2- methylpyridin-4-yl)morpholine | Method 6 |

| | | | |
|---|---|---|---|
| 59 | 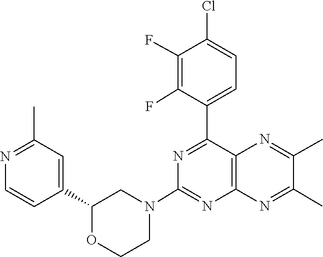 [SMILES: Cc1cc([C@@H]2CN(c3nc(-c4ccc(Cl)c(F)c4F)c4nc(C)c(C)nc4n3)CCO2)ccn1] | (R)-4-(4-(4-chloro-2,3- difluorophenyl)-6,7- dimethylpteridin-2-yl)-2-(2- methylpyridin-4-yl)morpholine | Method 6 |

| | | | |
|---|---|---|---|
| 60 | 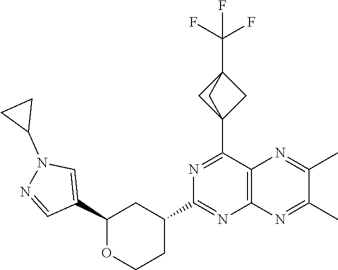 [SMILES: Cc1nc2nc([C@@H]3CCO[C@@H](c4cnn(C5CC5)c4)C3)nc(C34CC(C(F)(F)F)(C3)C4)c2nc1C] | 2-(2-(1-cyclopropyl-1H-pyrazol- 4-yl)tetrahydro-2H-pyran-4-yl)- 6,7-dimethyl-4-(3- (trifluoromethyl)bicyclo[1.1.1] pentan-1-yl)pteridine | Method 2 |

| | | | |
|---|---|---|---|
| 61 | 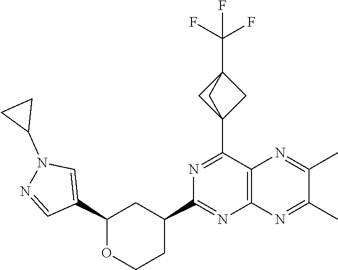 [SMILES: Cc1nc2nc([C@H]3CCO[C@@H](c4cnn(C5CC5)c4)C3)nc(C34CC(C(F)(F)F)(C3)C4)c2nc1C] | 2-(2-(1-cyclopropyl-1H-pyrazol- 4-yl)tetrahydro-2H-pyran-4-yl)- 6,7-dimethyl-4-(3- (trifluoromethyl)bicyclo[1.1.1] pentan-1-yl)pteridine | Method 2 |

| | | | |
|---|---|---|---|
| 62 | 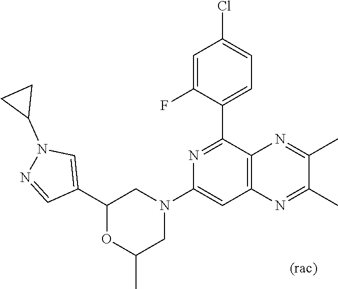 [SMILES: Cc1nc2cc(N3CC(C)OC(c4cnn(C5CC5)c4)C3)nc(-c3ccc(Cl)cc3F)c2nc1C] | 4-(5-(4-chloro-2-fluorophenyl)- 2,3-dimethylpyrido[3,4- b]pyrazin-7-yl)-2-(1- cyclopropyl-1H-pyrazol-4-yl)-6- methylmorpholine | Method 5 |

| | | | |
|---|---|---|---|
| 63 | 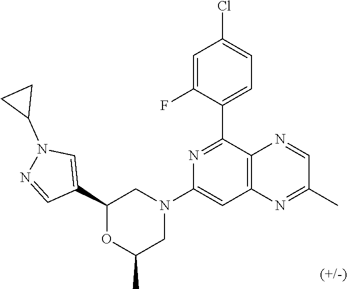 [SMILES: Cc1cnc2c(-c3ccc(Cl)cc3F)nc(N3C[C@@H](C)O[C@@H](c4cnn(C5CC5)c4)C3)cc2n1] | 4-(5-(4-chloro-2-fluorophenyl)- 2-methylpyrido[3,4-b]pyrazin-7- yl)-2-(1-cyclopropyl-1H- pyrazol-4-yl)-6- methylmorpholine | Method 5 |

| | | | |
|---|---|---|---|
| 64 | 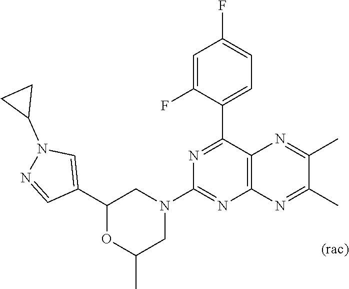 [SMILES: Cc1nc2nc(N3CC(C)OC(c4cnn(C5CC5)c4)C3)nc(-c3ccc(F)cc3F)c2nc1C] | 2-(1-cyclopropyl-1H-pyrazol-4- yl)-4-(4-(2,4-difluorophenyl)- 6,7-dimethylpteridin-2-yl)-6- methylmorpholine | Method 6 |

| | | | |
|---|---|---|---|
| 65 | 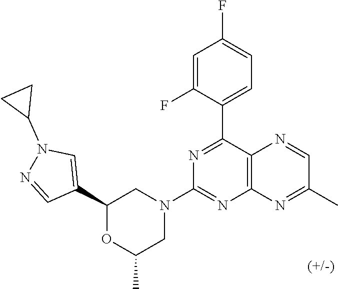 [SMILES: Cc1cnc2c(-c3ccc(F)cc3F)nc(N3C[C@H](C)O[C@@H](c4cnn(C5CC5)c4)C3)nc2n1] | 2-(1-cyclopropyl-1H-pyrazol-4- yl)-4-(4-(2,4-difluorophenyl)-7- methylpteridin-2-yl)-6- methylmorpholine | Method 6 |

| | | | |
|---|---|---|---|
| 66 | 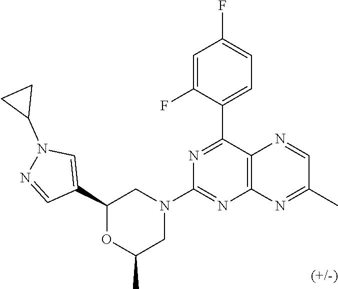 [SMILES: Cc1cnc2c(-c3ccc(F)cc3F)nc(N3C[C@@H](C)O[C@@H](c4cnn(C5CC5)c4)C3)nc2n1] | 2-(1-cyclopropyl-1H-pyrazol-4- yl)-4-(4-(2,4-difluorophenyl)-7- methylpteridin-2-yl)-6- methylmorpholine | Method 6 |

| | | | |
|---|---|---|---|
| 67 | 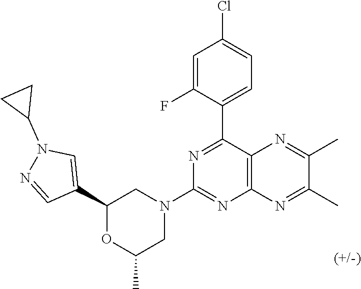 [SMILES: Cc1nc2nc(N3C[C@H](C)O[C@@H](c4cnn(C5CC5)c4)C3)nc(-c3ccc(Cl)cc3F)c2nc1C] | 4-(4-(4-chloro-2-fluorophenyl)- 6,7-dimethylpteridin-2-yl)-2-(1- cyclopropyl-1H-pyrazol-4-yl)-6- methylmorpholine | Method 6 |

| | | | |
|---|---|---|---|
| 68 | 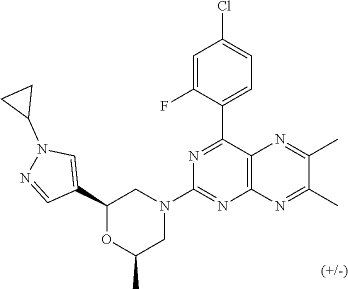 [SMILES: Cc1nc2nc(N3C[C@@H](C)O[C@@H](c4cnn(C5CC5)c4)C3)nc(-c3ccc(Cl)cc3F)c2nc1C] | 4-(4-(4-chloro-2-fluorophenyl)- 6,7-dimethylpteridin-2-yl)-2-(1- cyclopropyl-1H-pyrazol-4-yl)-6- methylmorpholine | Method 6 |

| | | | |
|---|---|---|---|
| 69 | 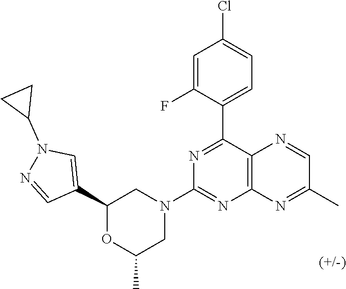 [SMILES: Cc1cnc2c(-c3ccc(Cl)cc3F)nc(N3C[C@H](C)O[C@@H](c4cnn(C5CC5)c4)C3)nc2n1] | 4-(4-(4-chloro-2-fluorophenyl)- 7-methylpteridin-2-yl)-2-(1- cyclopropyl-1H-pyrazol-4-yl)-6- methylmorpholine | Method 6 |

| | | | |
|---|---|---|---|
| 70 | 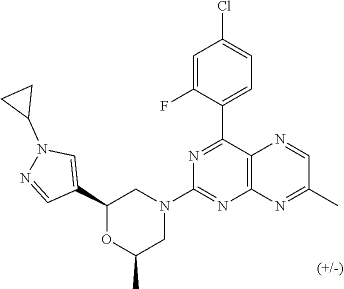 [SMILES: Cc1cnc2c(-c3ccc(Cl)cc3F)nc(N3C[C@@H](C)O[C@@H](c4cnn(C5CC5)c4)C3)nc2n1] | 4-(4-(4-chloro-2-fluorophenyl)- 7-methylpteridin-2-yl)-2-(1- cyclopropyl-1H-pyrazol-4-yl)-6- methylmorpholine | Method 6 |

| | | | |
|---|---|---|---|
| 71 | 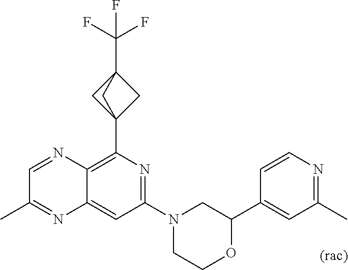 | 2-(2-methyl-4-pyridyl)-4-[2- methyl-5-[3-(trifluoromethyl)-1- bicyclo[1.1.1]pentanyl]pyrido[3, 4-b]pyrazin-7-yl]morpholine | Method 5 |
| 72 | 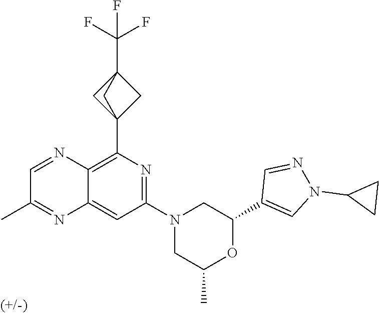 | 2-(1-cyclopropylpyrazol-4-yl)-6- methyl-4-[2-methyl-5-[3- (trifluoromethyl)-1- bicyclo[1.1.1]pentanyl]pyrido[3, 4-b]pyrazin-7-yl] morpholine | Method 5 |
| 73 | 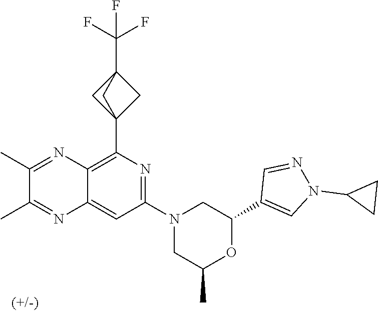 | 2-(1-cyclopropylpyrazol-4-yl)-4- [2,3-dimethyl-5-[3- (trifluoromethyl)-1- bicyclo[1.1.1]pentanyl]pyrido[3, 4-b]pyrazin-7-yl]-6-methyl- morpholine | Method 5 |
| 74 | 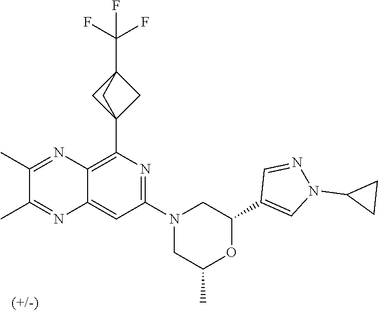 | 2-(1-cyclopropylpyrazol-4-yl)-4- [2,3-dimethyl-5-[3- (trifluoromethyl)-1- bicyclo[1.1.1]pentanyl]pyrido[3, 4-b]pyrazin-7-yl]-6-methyl- morpholine | Method 5 |
| 75 | 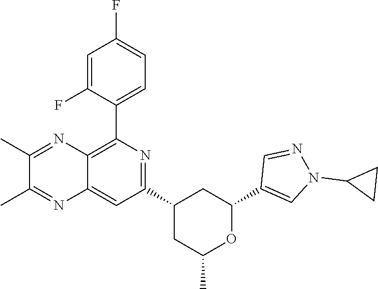 | 7-[ (2R,4S,6R)-2-(1- cyclopropylpyrazol-4-yl)-6- methyl-tetrahydropyran-4-yl]-5- (2,4-difluorophenyl)-2,3- dimethyl-pyrido[3,4-b]pyrazine | Method 1 |
| 76 | 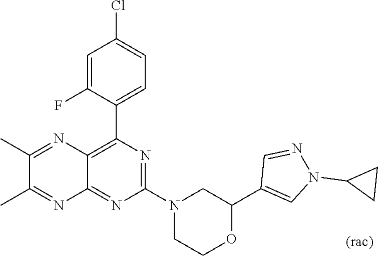 | 4-[ 4-(4-chloro-2-fluoro-phenyl)- 6,7-dimethyl-pteridin-2-yl]-2-(1- cyclopropylpyrazol-4- yl)morpholine | Method 6 |
| 77 | 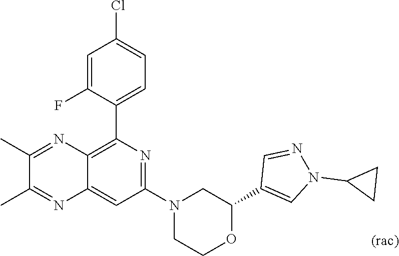 | (2S)-4-[5-(4-chloro-2-fluoro- phenyl)-2,3-dimethyl- pyrido[3,4-b]pyrazin-7-yl]-2-(1- cyclopropylpyrazol-4- yl)morpholine | Method 3 |
| 78 | 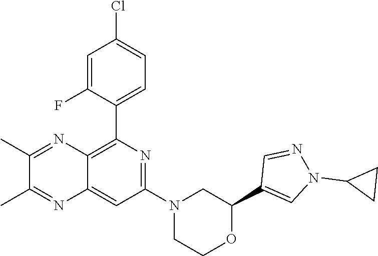 | (2R)-4-[5-(4-chloro-2-fluoro- phenyl)-2,3-dimethyl- pyrido[3,4-b]pyrazin-7-yl]-2-(1- cyclopropylpyrazol-4- yl)morpholine | Method 3 |
| 79 | 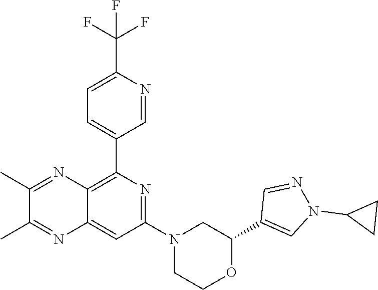 | (2S)-2-(1-cyclopropylpyrazol-4- yl)-4-[2,3-dimethyl-5-[6- (trifluoromethyl)-3- pyridyl]pyrido[3,4-b]pyrazin-7- yl] morpholine | Method 3 |
| 80 | 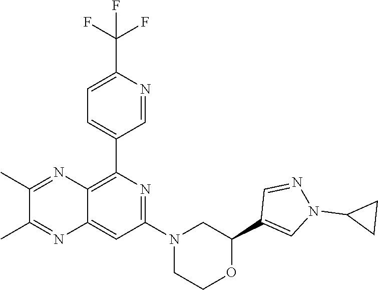 | (2R)-2-(1-cyclopropylpyrazol-4- yl)-4-[2,3-dimethyl-5-[6- (trifluoromethyl)-3- pyridyl]pyrido[3,4-b]pyrazin-7- yl]morpholine | Method 3 |
| 81 | 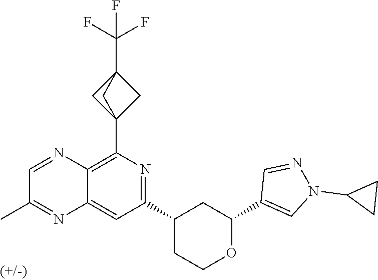 | 2-methyl-7-[2-(1- cyclopropylpyrazol-4- yl)tetrahydropyran-4-yl]-5-[3- (trifluoromethyl)-1- bicyclo[1.1.1]pentanyl]pyrido[3, 4-b]pyrazine | Method 8 |
| 82 | 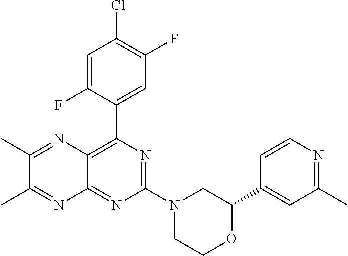 | (2S)-4-[ 4-(4-chloro-2,5-difluoro- phenyl)-6,7-dimethyl-pteridin-2- yl]-2-(2-methyl-4- pyridyl)morpholine | Method 6 |
| 83 | 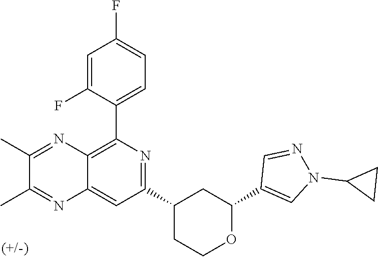 | 5-(2,4-difluorophenyl)-2-methyl- 7-[ 2-(1-cyclopropylpyrazol-4- yl)tetrahydropyran-4- yl]pyrido[3,4-b]pyrazine | Method 8 |
| 84 | 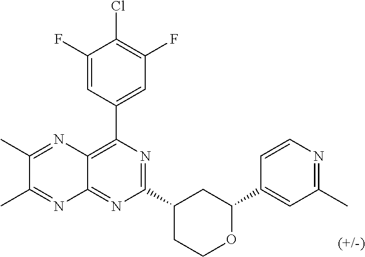 | 4-(4-chloro-3,5-difluoro- phenyl)-6,7-dimethyl-2-[2-(2- methyl-4- pyridyl)tetrahydropyran-4- yl]pteridine | Method 7 |
| 85 | 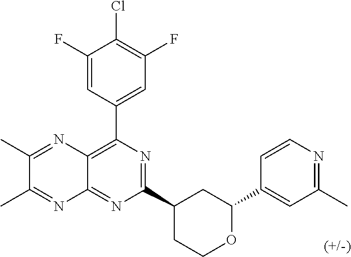 | 4-(4-chloro-3,5-difluoro- phenyl)-6,7-dimethyl-2-[2-(2- methyl-4- pyridyl)tetrahydropyran-4- yl]pteridine | Method 7 |
| 86 | 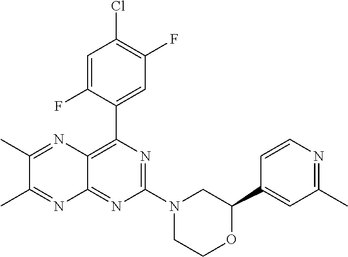 | (2R)-4-[ 4-(4-chloro-2,5- difluoro-phenyl)-6,7-dimethyl- pteridin-2-yl]-2-(2-methyl-4- pyridyl)morpholine | Method 6 |
| 87 | 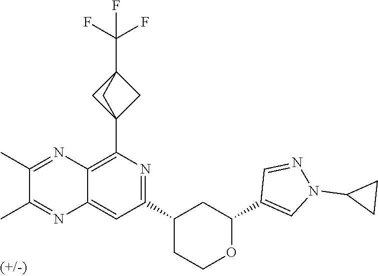 | 2,3-dimethyl-7-[ 2-(1- cyclopropylpyrazol-4- yl)tetrahydropyran-4-yl]-5-[3- (trifluoromethyl)-1- bicyclo[1.1.1]pentanyl]pyrido[3, 4-b]pyrazine | Method 8 |
| 88 | 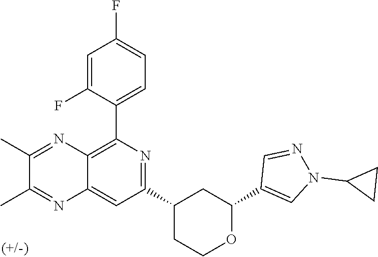 | 7-[ 2-(1-cyclopropylpyrazol-4- yl)tetrahydropyran-4-yl]-5-(2,4- difluorophenyl)-2,3-dimethyl- pyrido[3,4-b]pyrazine | Method 8 |
| 89 | 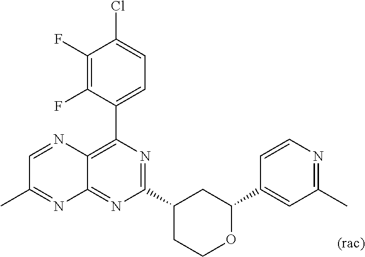 | 4-(4-chloro-2,3-difluoro- phenyl)-7-methyl-2-[2-(2- methyl-4- pyridyl)tetrahydropyran-4- yl]pteridine | Method 9 |
| 90 | 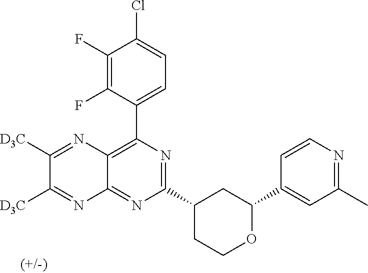 [SMILES: [2H]C([2H])([2H])c1nc2nc([C@H]3CCO[C@@H](c4ccnc(C)c4)C3)nc(-c3ccc(Cl)c(F)c3F)c2nc1C([2H])([2H])[2H]] | 4-(4-chloro-2,3-difluoro- phenyl)-2-[ 2-(2-methyl-4- pyridyl)tetrahydropyran-4-yl]- 6,7- bis(trideuteriomethyl)pteridine | Method 9 |
| 91 | 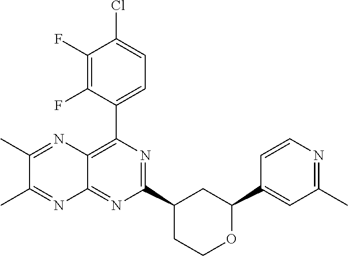 | 4-(4-chloro-2,3-difluorophenyl)- 6,7-dimethyl-2-((2S,4R)-2-(2- methylpyridin-4-yl)tetrahydro- 2H-pyran-4-yl)pteridine | Method 7 |
| 92 | 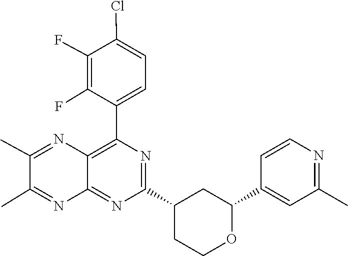 | 4-(4-chloro-2,3-difluorophenyl)- 6,7-dimethyl-2-((2R,4S)-2-(2- methylpyridin-4-yl)tetrahydro- 2H-pyran-4-yl)pteridine | Method 7 |
| 93 | 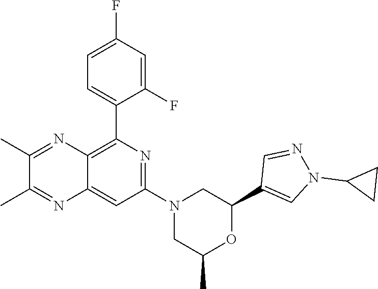 | (2R,6S)-2-(1-cyclopropyl-1H- pyrazol-4-yl)-4-(5-(2,4- difluorophenyl)-2,3- dimethylpyrido[3,4-b]pyrazin-7- yl)-6-methylmorpholine | Method 5 |
| 94 | 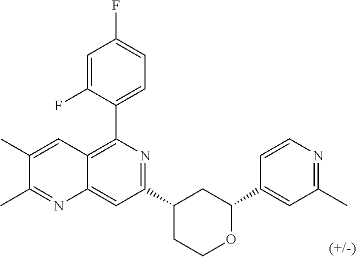 | 5-(2,4-difluorophenyl)-2,3- dimethyl-7-[2-(2-methyl-4- pyridyl)tetrahydropyran-4-yl]- 1,6-naphthyridine | |
| 95 | 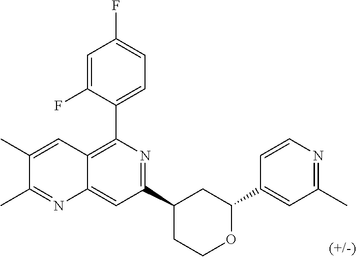 | 5-(2,4-difluorophenyl)-2,3- dimethyl-7-[2-(2-methyl-4- pyridyl)tetrahydropyran-4-yl]- 1,6-naphthyridine | |
| 96 | 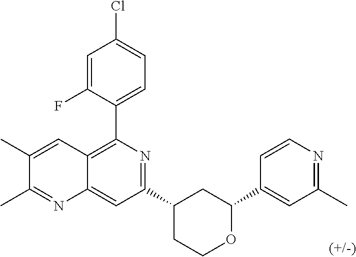 | 5-(4-chloro-2-fluoro-phenyl)- 2,3-dimethyl-7-[ 2-(2-methyl-4- pyridyl)tetrahydropyran-4-yl]- 1,6-naphthyridine | |
| 97 | 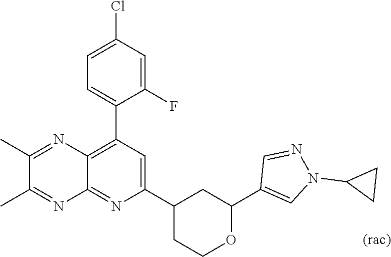 | 8-(4-chloro-2-fluorophenyl)-6- (2-(1-cyclopropyl-1H-pyrazol-4- yl)tetrahydro-2H-pyran-4-yl)- 2,3-dimethylpyrido[2,3- b]pyrazine | Method 10 |
| 98 | 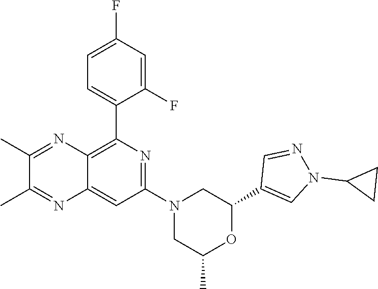 | (2S,6R)-2-(1-cyclopropyl-1H- pyrazol-4-yl)-4-(5-(2,4- difluorophenyl)-2,3- dimethylpyrido[3,4-b]pyrazin-7- yl)-6-methylmorpholine | Method 5 |
| 99 | 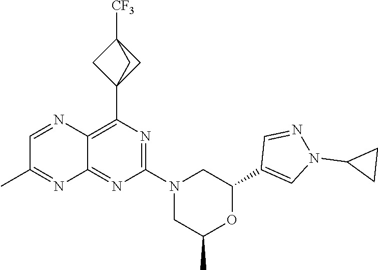 [SMILES: Cc1cnc2c(C34CC(C(F)(F)F)(C3)C4)nc(N3C[C@H](C)O[C@@H](c4cnn(C5CC5)c4)C3)nc2n1] | (2S,6S)-2-(1-cyclopropyl-1H- pyrazol-4-yl)-6-methyl-4-(7- methyl-4-(3- (trifluoromethyl)bicyclo[1.1.1] pentan-1-yl)pteridin-2- yl)morpholine | Method 6 |
| 100 | 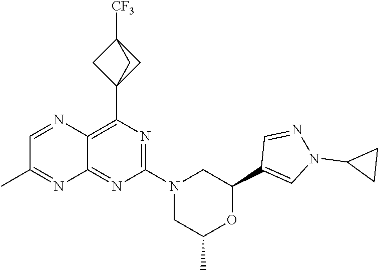 | (2R,6R)-2-(1-cyclopropyl-1H- pyrazol-4-yl)-6-methyl-4-(7- methyl-4-(3- (trifluoromethyl)bicyclo[1.1.1] pentan-1-yl)pteridin-2- yl)morpholine | Method 6 |
| 101 | 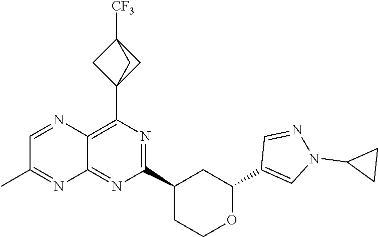 | 2-((2R,4R)-2-(1-cyclopropyl- 1H-pyrazol-4-yl)tetrahydro-2H- pyran-4-yl)-7-methyl-4-(3- (trifluoromethyl)bicyclo[1.1.1] pentan-1-yl)pteridine | Method 2 |
| 102 | 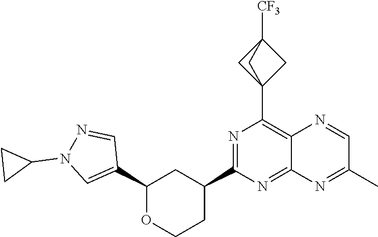 | 2-((2R,4S)-2-(1-cyclopropyl-1H- pyrazol-4-yl)tetrahydro-2H- pyran-4-yl)-7-methyl-4-(3- (trifluoromethyl)bicyclo[1.1.1] pentan-1-yl)pteridine | Method 2 |
| 103 | 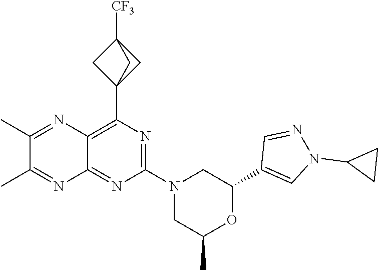 | (2S,6S)-2-(1-cyclopropyl-1H- pyrazol-4-yl)-4-(6,7-dimethyl-4- (3- (trifluoromethyl)bicyclo[1.1.1] pentan-1-yl)pteridin-2-yl)-6- methylmorpholine | Method 6 |
| 104 | 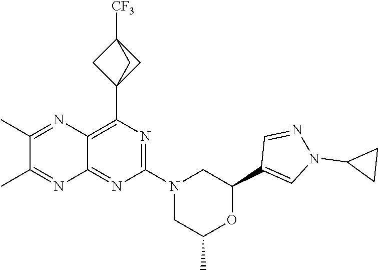 | (2R,6R)-2-(1-cyclopropyl-1H- pyrazol-4-yl)-4-(6,7-dimethyl-4- (3- (trifluoromethyl)bicyclo[1.1.1] pentan-1-yl)pteridin-2-yl)-6- methylmorpholine | Method 6 |
| 105 | 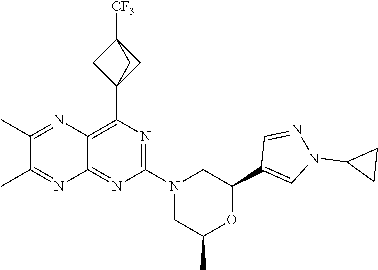 | (2R,6S)-2-(1-cyclopropyl-1H- pyrazol-4-yl)-4-(6,7-dimethyl-4- (3- (trifluoromethyl)bicyclo[1.1.1] pentan-1-yl)pteridin-2-yl)-6- methylmorpholine | Method 6 |
| 106 | 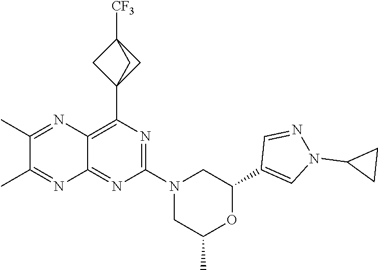 | (2S,6R)-2-(1-cyclopropyl-1H- pyrazol-4-yl)-4-(6,7-dimethyl-4- (3- (trifluoromethyl)bicyclo[1.1.1] pentan-1-yl)pteridin-2-yl)-6- methylmorpholine | Method 6 |
| 107 | 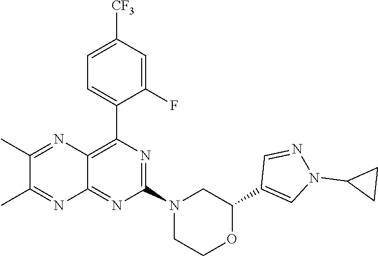 | 2-((2R,4R)-2-(1-cyclopropyl- 1H-pyrazol-4-yl)tetrahydro-2H- pyran-4-yl)-4-(2-fluoro-4- (trifluoromethyl)phenyl)-6,7- dimethylpteridine | Method 2 |
| 108 | 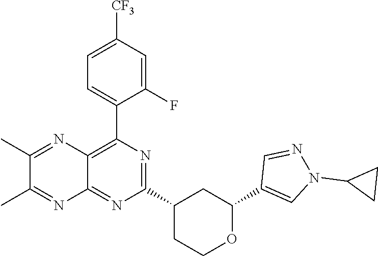 | 2-((2R,4S)-2-(1-cyclopropyl-1H- pyrazol-4-yl)tetrahydro-2H- pyran-4-yl)-4-(2-fluoro-4- (trifluoromethyl)phenyl)-6,7- dimethylpteridine | Method 2 |
| 109 | 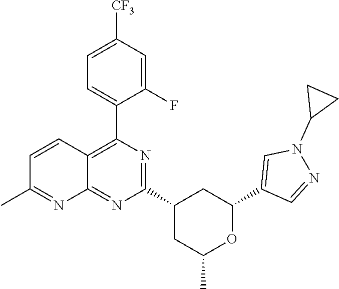 | 4-(4-chloro-2-fluorophenyl)-2- ((2R,4S,6R)-2-(1-cyclopropyl- 1H-pyrazol-4-yl)-6- methyltetrahydro-2H-pyran-4- yl)-7-methylpyrido[ 2,3- d]pyrimidine | |
| 110 | 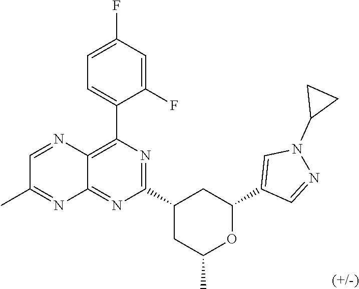 | 2-((2R,4S,6R)-2-(1-cyclopropyl- 1H-pyrazol-4-yl)-6- methyltetrahydro-2H-pyran-4- yl)-4-(2,4-difluorophenyl)-7- methylpteridine | |
| 111 | 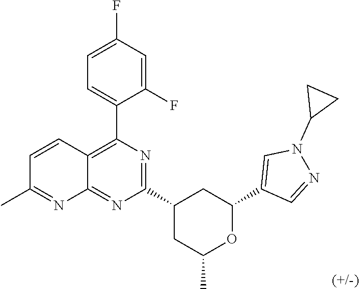 | 2-((2R,4S,6R)-2-(1-cyclopropyl- 1H-pyrazol-4-yl)-6- methyltetrahydro-2H-pyran-4- yl)-4-(2,4-difluorophenyl)-7- methylpyrido[ 2,3-d]pyrimidine | |
| 112 | 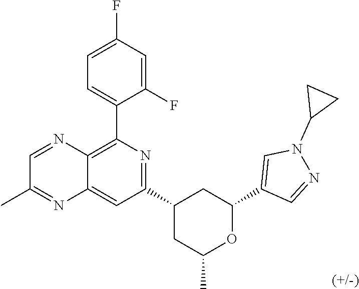 | 7-((2R,4S,6R)-2-(1-cyclopropyl- 1H-pyrazol-4-yl)-6- methyltetrahydro-2H-pyran-4- yl)-5-(2,4-difluorophenyl)-2- methylpyrido[ 3,4-b]pyrazine | |
| 113 | 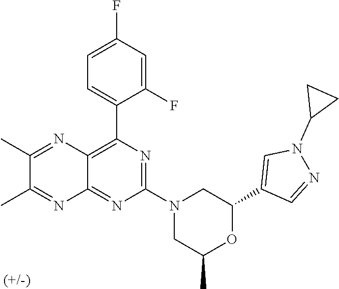 | 2-(1-cyclopropyl-1H-pyrazol-4- yl)-4-(4-(2,4-difluorophenyl)- 6,7-dimethylpteridin-2-yl)-6- methylmorpholine | Method 6 |
| 114 | 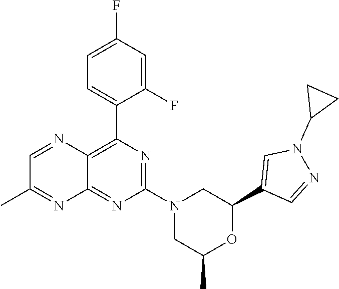 | (2R,6S)-2-(1-cyclopropyl-1H- pyrazol-4-yl)-4-(4-(2,4- difluorophenyl)-7- methylpteridin-2-yl)-6- methylmorpholine | Method 6 |
| 115 | 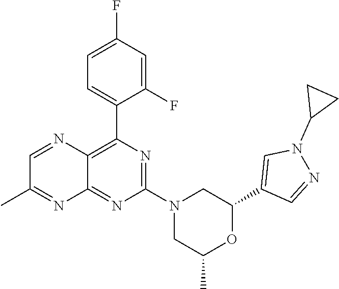 | (2S,6R)-2-(1-cyclopropyl-1H- pyrazol-4-yl)-4-(4-(2,4- difluorophenyl)-7- methylpteridin-2-yl)-6- methylmorpholine | Method 6 |
| 116 | 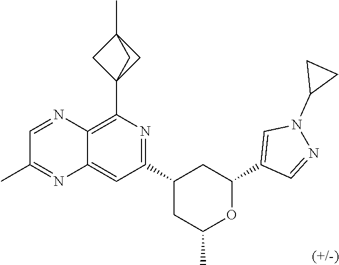 | 7-((2R,4S,6R)-2-(1-cyclopropyl- 1H-pyrazol-4-yl)-6- methyltetrahydro-2H-pyran-4- yl)-2-methyl-5-(3- methylbicyclo[1.1.1]pentan-1- yl)pyrido[ 3,4-b]pyrazine | |
| 117 | 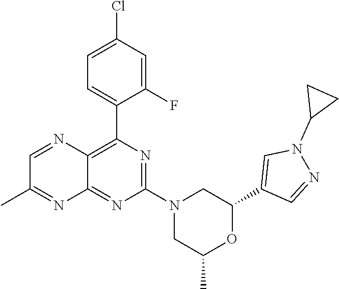 | (2S,6R)-4-(4-(4-chloro-2- fluorophenyl)-7-methylpteridin- 2-yl)-2-(1-cyclopropyl-1H- pyrazol-4-yl)-6- methylmorpholine | Method 6 |
| 118 | 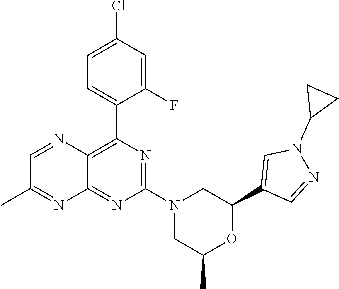 | (2R,6S)-4-(4-(4-chloro-2- fluorophenyl)-7-methylpteridin- 2-yl)-2-(1-cyclopropyl-1H- pyrazol-4-yl)-6- methylmorpholine | Method 6 |
| 119 | 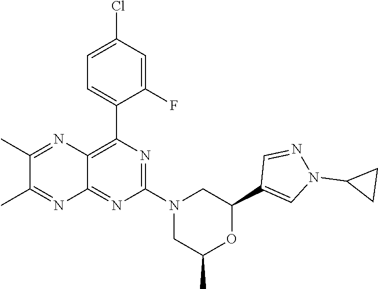 [SMILES: Cc1nc2nc(N3C[C@@H](c4cnn(C5CC5)c4)O[C@@H](C)C3)nc(-c3ccc(Cl)cc3F)c2nc1C] | (2R,6S)-4-(4-(4-chloro-2- fluorophenyl)-6,7- dimethylpteridin-2-yl)-2-(1- cyclopropyl-1H-pyrazol-4-yl)-6- methylmorpholine | Method 6 |
| 120 | 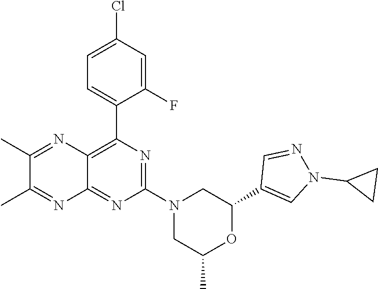 [SMILES: Cc1nc2nc(N3C[C@@H](C)O[C@@H](c4cnn(C5CC5)c4)C3)nc(-c3ccc(Cl)cc3F)c2nc1C] | (2S,6R)-4-(4-(4-chloro-2- fluorophenyl)-6,7- dimethylpteridin-2-yl)-2-(1- cyclopropyl-1H-pyrazol-4-yl)-6- methylmorpholine | Method 6 |
| 121 | 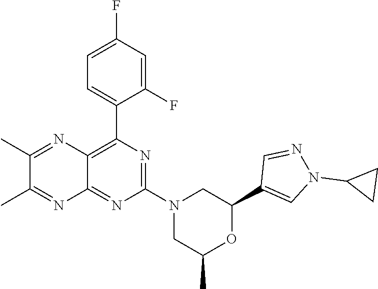 [SMILES: Cc1nc2nc(N3C[C@@H](c4cnn(C5CC5)c4)O[C@@H](C)C3)nc(-c3ccc(F)cc3F)c2nc1C] | (2R,6S)-2-(1-cyclopropyl-1H- pyrazol-4-yl)-4-(4-(2,4- difluorophenyl)-6,7- dimethylpteridin-2-yl)-6- methylmorpholine | Method 6 |
| 122 | 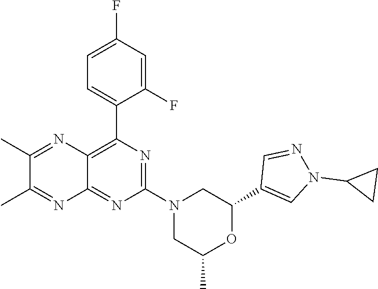 [SMILES: Cc1nc2nc(N3C[C@@H](C)O[C@@H](c4cnn(C5CC5)c4)C3)nc(-c3ccc(F)cc3F)c2nc1C] | (2S,6R)-2-(1-cyclopropyl-1H- pyrazol-4-yl)-4-(4-(2,4- difluorophenyl)-6,7- dimethylpteridin-2-yl)-6- methylmorpholine | Method 6 |
| 123 | 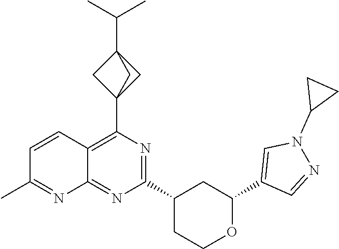 [SMILES: Cc1ccc2c(C34CC(C(C)C)(C3)C4)nc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-4-(3- isopropylbicyclo[1.1.1]pentan-1- yl)-7-methylpyrido[ 2,3- d]pyrimidine | |
| 124 | 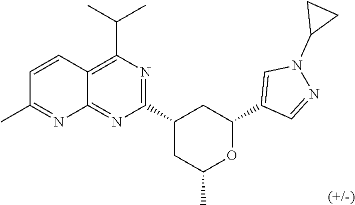 [SMILES: Cc1ccc2c(C(C)C)nc([C@H]3C[C@@H](C)O[C@@H](c4cnn(C5CC5)c4)C3)nc2n1] | 2-((2R,4S,6R)-2-(1-cyclopropyl- 1H-pyrazol-4-yl)-6- methyltetrahydro-2H-pyran-4- yl)-4-isopropyl-7- methylpyrido[2,3-d]pyrimidine | |
| 125 | 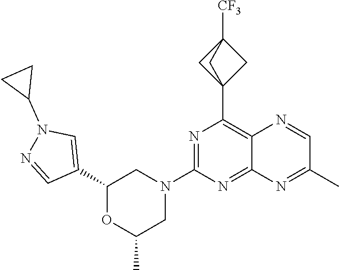 [SMILES: Cc1cnc2c(C34CC(C(F)(F)F)(C3)C4)nc(N3C[C@@H](c4cnn(C5CC5)c4)O[C@@H](C)C3)nc2n1] | (2R,6S)-2-(1-cyclopropyl-1H- pyrazol-4-yl)-6-methyl-4-(7- methyl-4-(3- (trifluoromethyl)bicyclo[1.1.1] pentan-1-yl)pteridin-2- yl)morpholine | Method 6 |
| 126 | 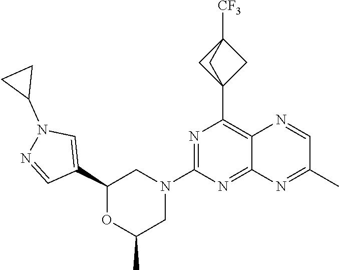 [SMILES: Cc1cnc2c(C34CC(C(F)(F)F)(C3)C4)nc(N3C[C@@H](C)O[C@@H](c4cnn(C5CC5)c4)C3)nc2n1] | (2S,6R)-2-(1-cyclopropyl-1H- pyrazol-4-yl)-6-methyl-4-(7- methyl-4-(3- (trifluoromethyl)bicyclo[1.1.1] pentan-1-yl)pteridin-2- yl)morpholine | Method 6 |
| 127 | 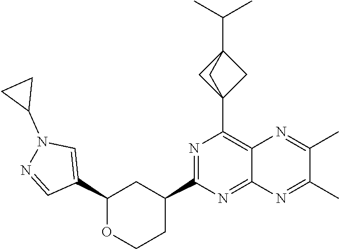 [SMILES: Cc1nc2nc([C@H]3CCO[C@@H](c4cnn(C5CC5)c4)C3)nc(C34CC(C(C)C)(C3)C4)c2nc1C] | 2-((2R,4S)-2-(1-cyclopropyl-1H- pyrazol-4-yl)tetrahydro-2H- pyran-4-yl)-4-(3- isopropylbicyclo[1.1.1]pentan-1- yl)-6,7-dimethylpteridine | |
| 128 | 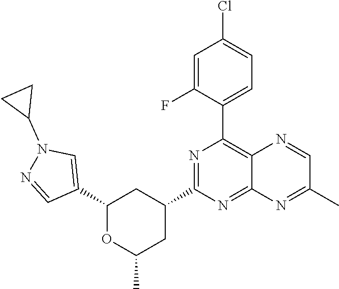 [SMILES: Cc1cnc2c(-c3ccc(Cl)cc3F)nc([C@H]3C[C@@H](c4cnn(C5CC5)c4)O[C@@H](C)C3)nc2n1] | 4-(4-chloro-2-fluorophenyl)-2- ((2S,4R,6S)-2-(1-cyclopropyl- 1H-pyrazol-4-yl)-6- methyltetrahydro-2H-pyran-4- yl)-7-methylpteridine | |
| 129 | 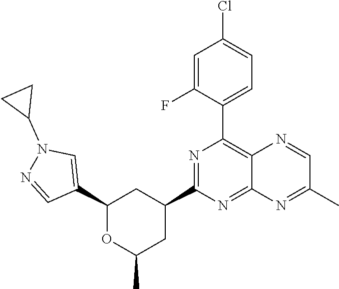 [SMILES: Cc1cnc2c(-c3ccc(Cl)cc3F)nc([C@H]3C[C@@H](C)O[C@@H](c4cnn(C5CC5)c4)C3)nc2n1] | 4-(4-chloro-2-fluorophenyl)-2- ((2R,4S,6R)-2-(1-cyclopropyl- 1H-pyrazol-4-yl)-6- methyltetrahydro-2H-pyran-4- yl)-7-methylpteridine | |
| 130 | 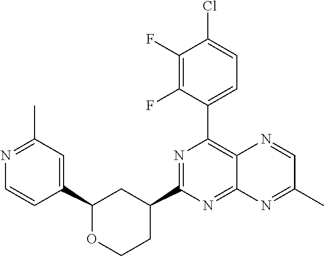 [SMILES: Cc1cc([C@H]2C[C@@H](c3nc(-c4ccc(Cl)c(F)c4F)c4ncc(C)nc4n3)CCO2)ccn1] | 4-(4-chloro-2,3-difluorophenyl)- 7-methyl-2-((2R,4S)-2-(2- methylpyridin-4-yl)tetrahydro- 2H-pyran-4-yl)pteridine | |
| 131 | 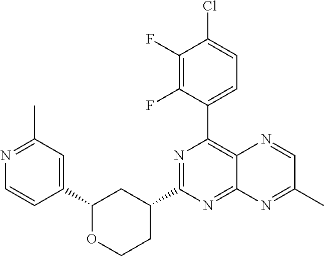 [SMILES: Cc1cc([C@@H]2C[C@H](c3nc(-c4ccc(Cl)c(F)c4F)c4ncc(C)nc4n3)CCO2)ccn1] | 4-(4-chloro-2,3-difluorophenyl)- 7-methyl-2-((2S,4R)-2-(2- methylpyridin-4-yl)tetrahydro- 2H-pyran-4-yl)pteridine | |
| 132 | 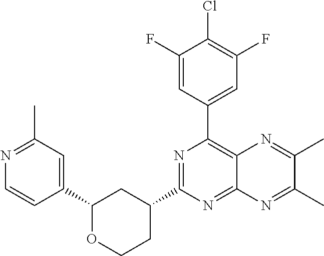 [SMILES: Cc1cc([C@@H]2C[C@H](c3nc(-c4cc(F)c(Cl)c(F)c4)c4nc(C)c(C)nc4n3)CCO2)ccn1] | 4-(4-chloro-3,5-difluorophenyl)- 6,7-dimethyl-2-((2S,4R)-2-(2- methylpyridin-4-yl)tetrahydro- 2H-pyran-4-yl)pteridine | |
| 133 | 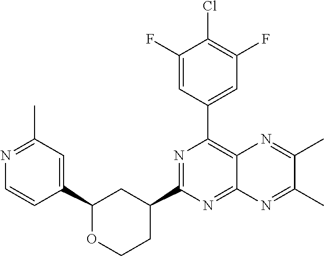 [SMILES: Cc1cc([C@H]2C[C@@H](c3nc(-c4cc(F)c(Cl)c(F)c4)c4nc(C)c(C)nc4n3)CCO2)ccn1] | 4-(4-chloro-3,5-difluorophenyl)- 6,7-dimethyl-2-((2R,4S)-2-(2- methylpyridin-4-yl)tetrahydro- 2H-pyran-4-yl)pteridine | |
| 134 | 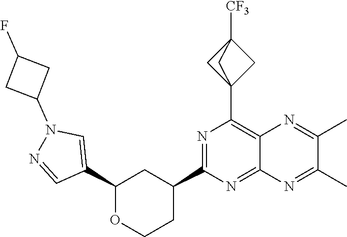 [SMILES: Cc1nc2nc([C@H]3CCO[C@@H](c4cnn(C5CC(F)C5)c4)C3)nc(C34CC(C(F)(F)F)(C3)C4)c2nc1C] | 2-((2S,4R)-2-(1-(3- fluorocyclobutyl)-1H-pyrazol-4- yl)tetrahydro-2H-pyran-4-yl)- 6,7-dimethyl-4-(3- (trifluoromethyl)bicyclo[1.1.1] pentan-1-yl)pteridine | Method 8 |
| 135 | 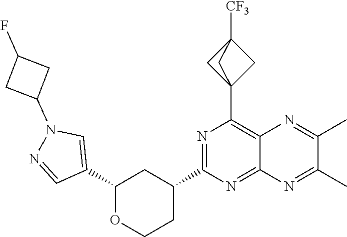 [SMILES: Cc1nc2nc([C@@H]3CCO[C@H](c4cnn(C5CC(F)C5)c4)C3)nc(C34CC(C(F)(F)F)(C3)C4)c2nc1C] | 2-((2R,4S)-2-(1-(3- fluorocyclobutyl)-1H-pyrazol-4- yl)tetrahydro-2H-pyran-4-yl)- 6,7-dimethyl-4-(3- (trifluoromethyl)bicyclo[1.1.1] pentan-1-yl)pteridine | Method 8 |
| 136 | 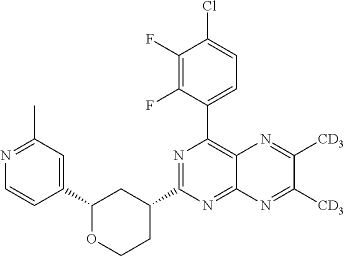 [SMILES: [2H]C([2H])([2H])c1nc2nc([C@@H]3CCO[C@H](c4ccnc(C)c4)C3)nc(-c3ccc(Cl)c(F)c3F)c2nc1C([2H])([2H])[2H]] | 4-(4-chloro-2,3-difluorophenyl)- 6,7-bis(methyl-d3)-2-((2S,4R)-2- (2-methylpyridin-4- yl)tetrahydro-2H-pyran-4- yl)pteridine | |
| 137 | 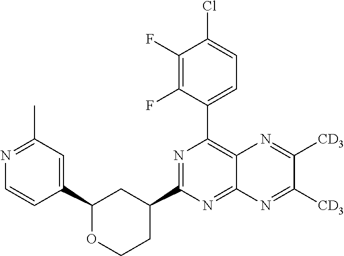 [SMILES: [2H]C([2H])([2H])c1nc2nc([C@H]3CCO[C@@H](c4ccnc(C)c4)C3)nc(-c3ccc(Cl)c(F)c3F)c2nc1C([2H])([2H])[2H]] | 4-(4-chloro-2,3-difluorophenyl)- 6,7-bis(methyl-d3)-2-((2R,4S)-2- (2-methylpyridin-4- yl)tetrahydro-2H-pyran-4- yl)pteridine | |
| 138 | 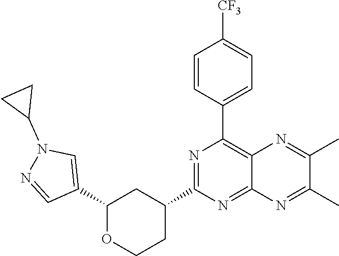 [SMILES: Cc1nc2nc([C@@H]3CCO[C@H](c4cnn(C5CC5)c4)C3)nc(-c3ccc(C(F)(F)F)cc3)c2nc1C] | 2-((2S,4R)-2-(1-cyclopropyl-1H- pyrazol-4-yl)tetrahydro-2H- pyran-4-yl)-6,7-dimethyl-4-(4- (trifluoromethyl)phenyl)pteridine | Method 8 |
| 139 | 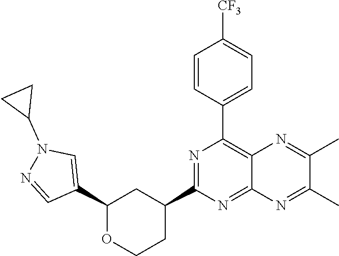 [SMILES: Cc1nc2nc([C@H]3CCO[C@@H](c4cnn(C5CC5)c4)C3)nc(-c3ccc(C(F)(F)F)cc3)c2nc1C] | 2-((2R,4S)-2-(1-cyclopropyl-1H- pyrazol-4-yl)tetrahydro-2H- pyran-4-yl)-6,7-dimethyl-4-(4- trifluoromethyl)phenyl)pteridine | Method 8 |
| 140 | 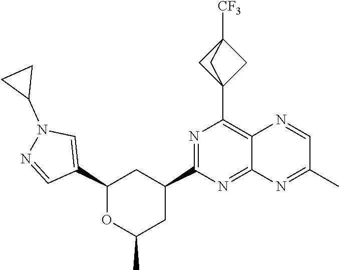 [SMILES: Cc1cnc2c(C34CC(C(F)(F)F)(C3)C4)nc([C@H]3C[C@@H](C)O[C@@H](c4cnn(C5CC5)c4)C3)nc2n1] | 2-((2R,4S,6R)-2-(1-cyclopropyl- 1H-pyrazol-4-yl)-6- methyltetrahydro-2H-pyran-4- yl)-7-methyl-4-(3- (trifluoromethyl)bicyclo[1.1.1] pentan-1-yl)pteridine | |
| 141 | 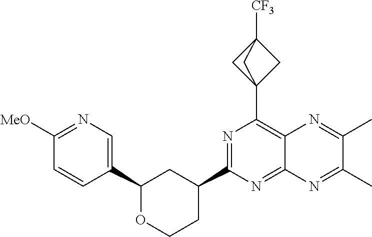 [SMILES: COc1ccc([C@H]2C[C@@H](c3nc(C45CC(C(F)(F)F)(C4)C5)c4nc(C)c(C)nc4n3)CCO2)cn1] | 2-((2R,4S)-2-(6- methoxypyridin-3-yl)tetrahydro- 2H-pyran-4-yl)-6,7-dimethyl-4- (3- (trifluoromethyl)bicyclo[1.1.1] pentan-1-yl)pteridine | Method 8 |
| 142 | 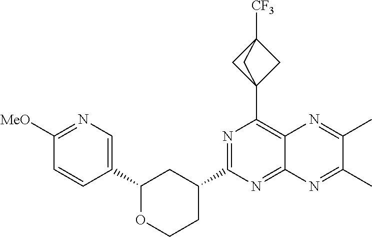 [SMILES: COc1ccc([C@@H]2C[C@H](c3nc(C45CC(C(F)(F)F)(C4)C5)c4nc(C)c(C)nc4n3)CCO2)cn1] | 2-((2S,4R)-2-(6- methoxypyridin-3-yl)tetrahydro- 2H-pyran-4-yl)-6,7-dimethyl-4- (3- (trifluoromethyl)bicyclo[1.1.1] pentan-1-yl)pteridine | Method 8 |
| 143 | 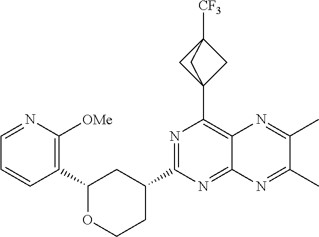 [SMILES: COc1ncccc1[C@@H]1C[C@H](c2nc(C34CC(C(F)(F)F)(C3)C4)c3nc(C)c(C)nc3n2)CCO1] | 2-((2S,4R)-2-(2- methoxypyridin-3-yl)tetrahydro- 2H-pyran-4-yl)-6,7-dimethyl-4- (3- (trifluoromethyl)bicyclo[1.1.1] pentan-1-yl)pteridine | Method 8 |
| 144 | 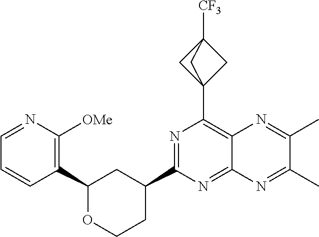 [SMILES: COc1ncccc1[C@H]1C[C@@H](c2nc(C34CC(C(F)(F)F)(C3)C4)c3nc(C)c(C)nc3n2)CCO1] | 2-((2R,4S)-2-(2- methoxypyridin-3-yl)tetrahydro- 2H-pyran-4-yl)-6,7-dimethyl-4- (3- (trifluoromethyl)bicyclo[1.1.1] pentan-1-yl)pteridine | Method 8 |
| 145 | 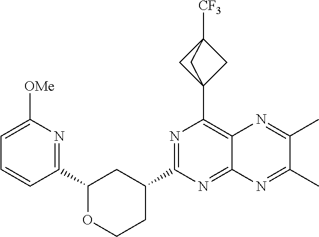 [SMILES: COc1cccc([C@@H]2C[C@H](c3nc(C45CC(C(F)(F)F)(C4)C5)c4nc(C)c(C)nc4n3)CCO2)n1] | 2-[ (2S,4R)-2-(6-methoxy-2- pyridyl)tetrahydropyran-4-yl] - 6,7-dimethyl-4-[ 3- (trifluoromethyl)-1- bicyclo[1.1.1]pentanyl]pteridine | Method 8 |
| 146 | 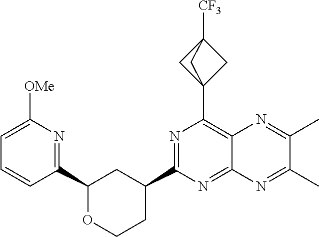 [SMILES: COc1cccc([C@H]2C[C@@H](c3nc(C45CC(C(F)(F)F)(C4)C5)c4nc(C)c(C)nc4n3)CCO2)n1] | 2-[ (2R,4S)-2-(6-methoxy-2- pyridyl)tetrahydropyran-4-yl] - 6,7-dimethyl-4-[ 3- (trifluoromethyl)-1- bicyclo[1.1.1]pentanyl]pteridine | Method 8 |
| 147 | 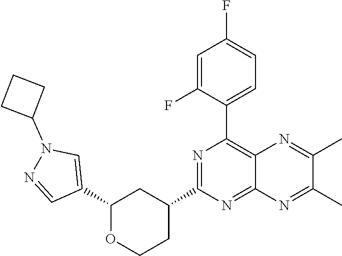 [SMILES: Cc1nc2nc([C@@H]3CCO[C@H](c4cnn(C5CCC5)c4)C3)nc(-c3ccc(F)cc3F)c2nc1C] | 2-((2S,4R)-2-(1-cyclobutyl-1H- pyrazol-4-yl)tetrahydro-2H- pyran-4-yl)-4-(2,4- difluorophenyl)-6,7- dimethylpteridine | Method 8 |
| 148 | 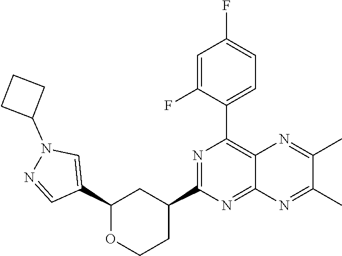 [SMILES: Cc1nc2nc([C@H]3CCO[C@@H](c4cnn(C5CCC5)c4)C3)nc(-c3ccc(F)cc3F)c2nc1C] | 2-((2R,4S)-2-(1-cyclobutyl-1H- pyrazol-4-yl)tetrahydro-2H- pyran-4-yl)-4-(2,4- difluorophenyl)-6,7- dimethylpteridine | Method 8 |
| 149 | 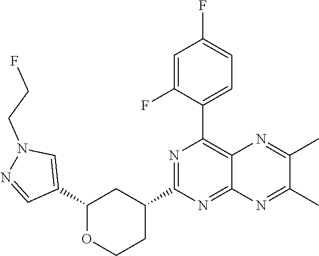 [SMILES: Cc1nc2nc([C@@H]3CCO[C@H](c4cnn(CCF)c4)C3)nc(-c3ccc(F)cc3F)c2nc1C] | 4-(2,4-difluorophenyl)-2- ((2S,4R)-2-(1-(2-fluoroethyl)- 1H-pyrazol-4-yl)tetrahydro-2H- pyran-4-yl)-6,7- dimethylpteridine | Method 8 |
| 150 | 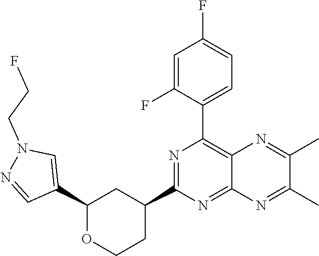 [SMILES: Cc1nc2nc([C@H]3CCO[C@@H](c4cnn(CCF)c4)C3)nc(-c3ccc(F)cc3F)c2nc1C] | 4-(2,4-difluorophenyl)-2- ((2R,4S)-2-(1-(2-fluoroethyl)- 1H-pyrazol-4-yl)tetrahydro-2H- pyran-4-yl)-6,7- dimethylpteridine | Method 8 |
| 151 | 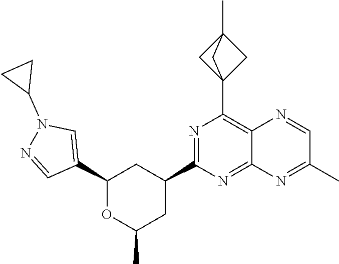 [SMILES: Cc1cnc2c(C34CC(C)(C3)C4)nc([C@H]3C[C@@H](C)O[C@@H](c4cnn(C5CC5)c4)C3)nc2n1] | 2-((2R,4S,6R)-2-(1-cyclopropyl- 1H-pyrazol-4-yl)-6- methyltetrahydro-2H-pyran-4- yl)-7-methyl-4-(3- methylbicyclo[1.1.1]pentan-1- yl)pteridine | |
| 152 | 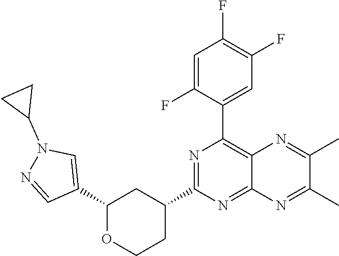 [SMILES: Cc1nc2nc([C@@H]3CCO[C@H](c4cnn(C5CC5)c4)C3)nc(-c3cc(F)c(F)cc3F)c2nc1C] | 2-((2S,4R)-2-(1-cyclopropyl-1H- pyrazol-4-yl)tetrahydro-2H- pyran-4-yl)-6,7-dimethyl-4- (2,4,5-trifluorophenyl)pteridine | Method 8 |
| 153 | 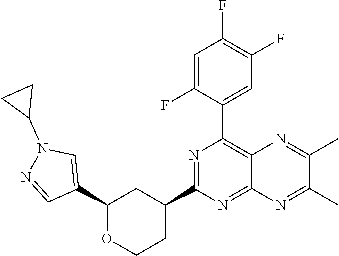 [SMILES: Cc1nc2nc([C@H]3CCO[C@@H](c4cnn(C5CC5)c4)C3)nc(-c3cc(F)c(F)cc3F)c2nc1C] | 2-((2R,4S)-2-(1-cyclopropyl-1H- pyrazol-4-yl)tetrahydro-2H- pyran-4-yl)-6,7-dimethyl-4- (2,4,5-trifluorophenyl)pteridine | Method 8 |
| 154 | 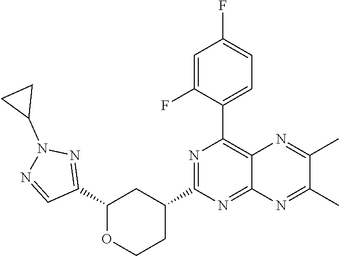 [SMILES: Cc1nc2nc([C@@H]3CCO[C@H](c4cnn(C5CC5)n4)C3)nc(-c3ccc(F)cc3F)c2nc1C] | 2-((2S,4R)-2-(2-cyclopropyl-2H- 1,2,3-triazol-4-yl)tetrahydro-2H- pyran-4-yl)-4-(2,4- difluorophenyl)-6,7- dimethylpteridine | Method 8 |
| 155 | 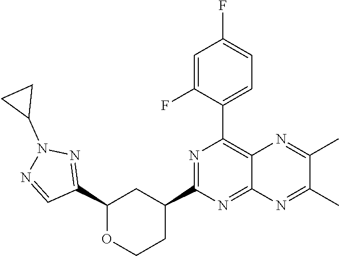 [SMILES: Cc1nc2nc([C@H]3CCO[C@@H](c4cnn(C5CC5)n4)C3)nc(-c3ccc(F)cc3F)c2nc1C] | 2-((2R,4S)-2-(2-cyclopropyl-2H- 1,2,3-triazol-4-yl)tetrahydro-2H- pyran-4-yl)-4-(2,4- difluorophenyl)-6,7- dimethylpteridine | Method 8 |
| 156 | 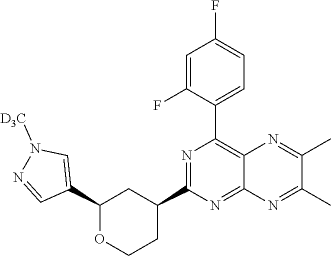 [SMILES: [2H]C([2H])([2H])n1cc([C@H]2C[C@@H](c3nc(-c4ccc(F)cc4F)c4nc(C)c(C)nc4n3)CCO2)cn1] | 4-(2-fluoro-4- (trifluoromethyl)phenyl)-6,7- dimethyl-2-((2R,4S)-2-(1- (methyl-d3)-1H-pyrazol-4- yl)tetrahydro-2H-pyran-4- yl)pteridine | |
| 157 | 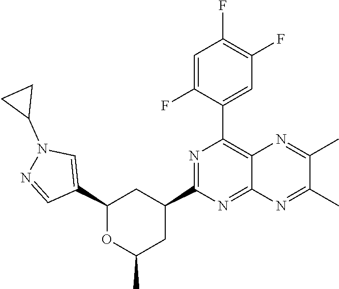 [SMILES: Cc1nc2nc([C@H]3C[C@@H](C)O[C@@H](c4cnn(C5CC5)c4)C3)nc(-c3cc(F)c(F)cc3F)c2nc1C] | 2-((2R,4S,6R)-2-(1-cyclopropyl- 1H-pyrazol-4-yl)-6- methyltetrahydro-2H-pyran-4- yl)-6,7-dimethyl-4-(2,4,5- trifluorophenyl)pteridine | Method 13 |
| 158 | 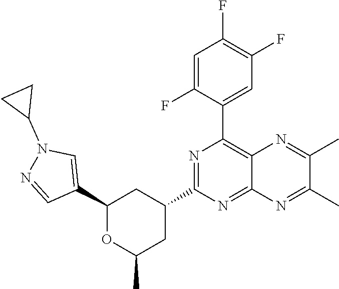 [SMILES: Cc1nc2nc([C@@H]3C[C@@H](C)O[C@@H](c4cnn(C5CC5)c4)C3)nc(-c3cc(F)c(F)cc3F)c2nc1C] | 2-((2R,4R,6R)-2-(1-cyclopropyl- 1H-pyrazol-4-yl)-6- methyltetrahydro-2H-pyran-4- yl)-6,7-dimethyl-4-(2,4,5- trifluorophenyl)pteridine | Method 13 |
| 159 | 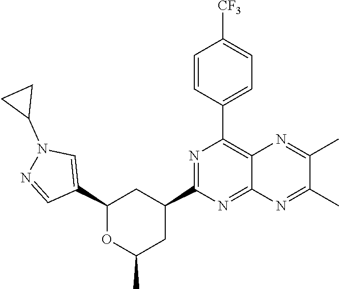 [SMILES: Cc1nc2nc([C@H]3C[C@@H](C)O[C@@H](c4cnn(C5CC5)c4)C3)nc(-c3ccc(C(F)(F)F)cc3)c2nc1C] | 2-((2R,4S,6R)-2-(1-cyclopropyl- 1H-pyrazol-4-yl)-6- methyltetrahydro-2H-pyran-4- yl)-6,7-dimethyl-4-(4- (trifluoromethyl)phenyl) pteridine | Method 13 |
| 160 | 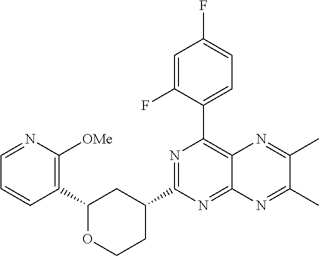 [SMILES: COc1ncccc1[C@@H]1C[C@H](c2nc(-c3ccc(F)cc3F)c3nc(C)c(C)nc3n2)CCO1] | 4-(2,4-difluorophenyl)-2- ((2S,4R)-2-(2-methoxypyridin- 3-yl)tetrahydro-2H-pyran-4-yl)- 6,7-dimethylpteridine | Method 8 |
| 161 | 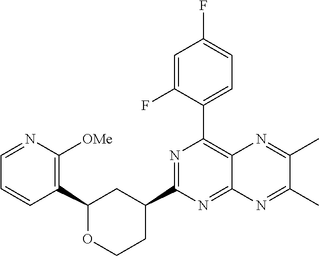 [SMILES: COc1ncccc1[C@H]1C[C@@H](c2nc(-c3ccc(F)cc3F)c3nc(C)c(C)nc3n2)CCO1] | 4-(2,4-difluorophenyl)-2- ((2R,4S)-2-(2-methoxypyridin- 3-yl)tetrahydro-2H-pyran-4-yl)- 6,7-dimethylpteridine | Method 8 |
| 162 | 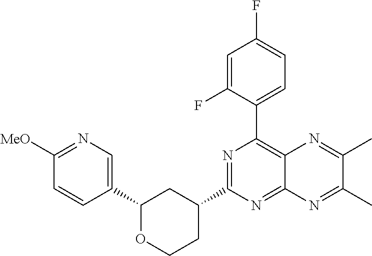 [SMILES: COc1ccc([C@@H]2C[C@H](c3nc(-c4ccc(F)cc4F)c4nc(C)c(C)nc4n3)CCO2)cn1] | 4-(2,4-difluorophenyl)-2- ((2S,4R)-2-(6-methoxypyridin- 3-yl)tetrahydro-2H-pyran-4-yl)- 6,7-dimethylpteridine | Method 8 |
| 163 | 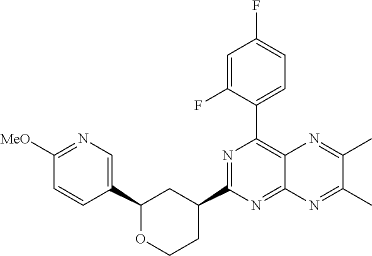 [SMILES: COc1ccc([C@H]2C[C@@H](c3nc(-c4ccc(F)cc4F)c4nc(C)c(C)nc4n3)CCO2)cn1] | 4-(2,4-difluorophenyl)-2- ((2R,4S)-2-(6-methoxypyridin- 3-yl)tetrahydro-2H-pyran-4-yl)- 6,7-dimethylpteridine | Method 8 |
| 164 | 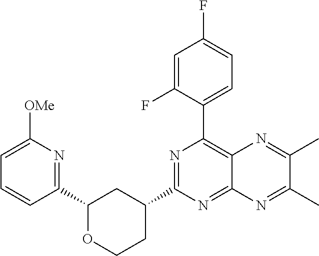 [SMILES: COc1cccc([C@@H]2C[C@H](c3nc(-c4ccc(F)cc4F)c4nc(C)c(C)nc4n3)CCO2)n1] | 4-(2,4-difluorophenyl)-2- ((2S,4R)-2-(6-methoxypyridin- 2-yl)tetrahydro-2H-pyran-4-yl)- 6,7-dimethylpteridine | Method 8 |
| 165 | 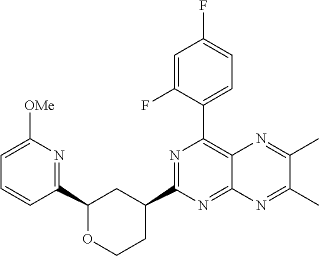 [SMILES: COc1cccc([C@H]2C[C@@H](c3nc(-c4ccc(F)cc4F)c4nc(C)c(C)nc4n3)CCO2)n1] | 4-(2,4-difluorophenyl)-2- ((2R,4S)-2-(6-methoxypyridin- 2-yl)tetrahydro-2H-pyran-4-yl)- 6,7-dimethylpteridine | Method 8 |
| 166 | 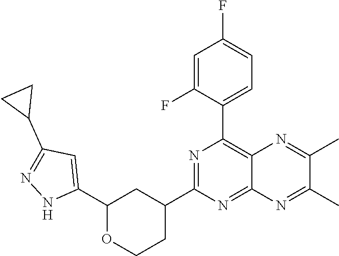 [SMILES: Cc1nc2nc(C3CCOC(c4cc(C5CC5)n[nH]4)C3)nc(-c3ccc(F)cc3F)c2nc1C] | 2-(2-(3-cyclopropyl-1H-pyrazol- 5-yl)tetrahydro-2H-pyran-4-yl)- 4-(2,4-difluorophenyl)-6,7- dimethylpteridine | Method 14 |
| 167 | 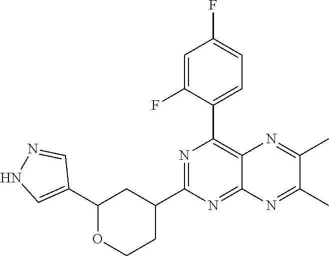 [SMILES: Cc1nc2nc(C3CCOC(c4cn[nH]c4)C3)nc(-c3ccc(F)cc3F)c2nc1C] | 2-(2-(1H-pyrazol-4- yl)tetrahydro-2H-pyran-4-yl)-4- (2,4-difluorophenyl)-6,7- dimethylpteridine | Method 14 |
| 168 | 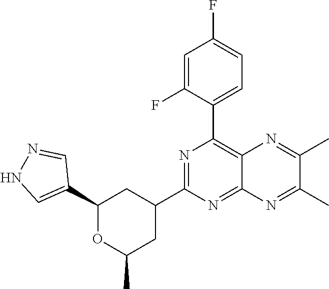 [SMILES: Cc1nc2nc(C3C[C@@H](C)O[C@@H](c4cn[nH]c4)C3)nc(-c3ccc(F)cc3F)c2nc1C] | 4-(2-fluoro-4- (trifluoromethyl)phenyl)-6,7- dimethyl-2-((2R,6R)-2-methyl- 6-(1H-pyrazol-4-yl)tetrahydro- 2H-pyran-4-yl)pteridine | Method 15 |
| 169 | 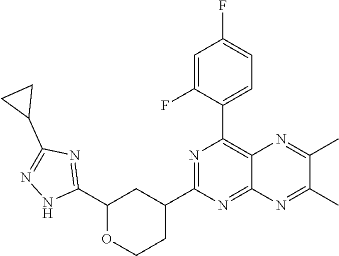 [SMILES: Cc1nc2nc(C3CCOC(c4nc(C5CC5)n[nH]4)C3)nc(-c3ccc(F)cc3F)c2nc1C] | 2-(2-(3-cyclopropyl-1H-1,2,4- triazol-5-yl)tetrahydro-2H- pyran-4-yl)-4-(2-fluoro-4- (trifluoromethyl)phenyl)-6,7- dimethylpteridine | Method 14 |
| 170 | 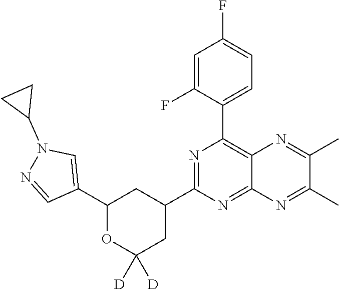 [SMILES: [2H]C1([2H])CC(c2nc(-c3ccc(F)cc3F)c3nc(C)c(C)nc3n2)CC(c2cnn(C3CC3)c2)O1] | 2-((2S,4R)-2-(1-cyclopropyl-1H- pyrazol-4-yl)tetrahydro-2H- pyran-4-yl-6,6-d2)-4-(2,4- difluorophenyl)-6,7- dimethylpteridine | |
| 171 | 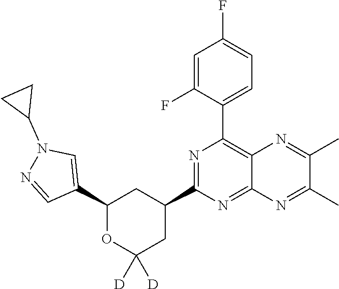 [SMILES: [2H]C1([2H])C[C@H](c2nc(-c3ccc(F)cc3F)c3nc(C)c(C)nc3n2)C[C@H](c2cnn(C3CC3)c2)O1] | 2-((2R,4S)-2-(1-cyclopropyl-1H- pyrazol-4-yl)tetrahydro-2H- pyran-4-yl-6,6-d2)-4-(2,4- difluorophenyl)-6,7- dimethylpteridine | |
| 172 | 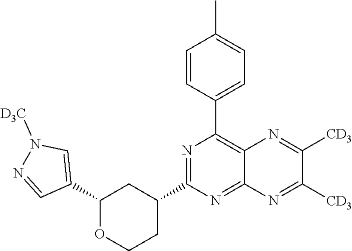 [SMILES: [2H]C([2H])([2H])c1nc2nc([C@@H]3CCO[C@H](c4cnn(C([2H])([2H])[2H])c4)C3)nc(-c3ccc(C)cc3)c2nc1C([2H])([2H])[2H]] | 6,7-bis(methyl-d3)-2-((2S,4R)-2- (1-(methyl-d3)-1H-pyrazol-4- yl)tetrahydro-2H-pyran-4-yl)-4- (p-tolyl)pteridine | |
| 173 | 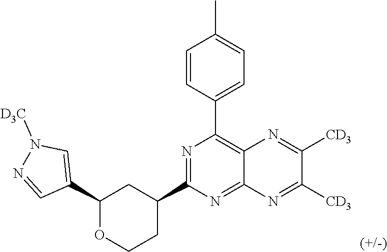 [SMILES: [2H]C([2H])([2H])c1nc2nc([C@H]3CCO[C@@H](c4cnn(C([2H])([2H])[2H])c4)C3)nc(-c3ccc(C)cc3)c2nc1C([2H])([2H])[2H]] | 6,7-bis(methyl-d3)-2-((2R,4S)-2- (1-(methyl-d3)-1H-pyrazol-4- yl)tetrahydro-2H-pyran-4-yl)-4- (p-tolyl)pteridine | |
| 174 | 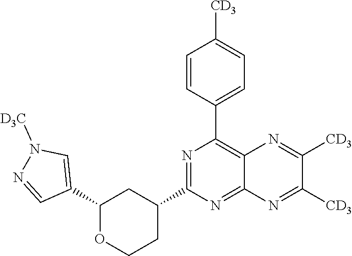 [SMILES: [2H]C([2H])([2H])c1ccc(-c2nc([C@@H]3CCO[C@H](c4cnn(C([2H])([2H])[2H])c4)C3)nc3nc(C([2H])([2H])[2H])c(C([2H])([2H])[2H])nc23)cc1] | 6,7-bis(methyl-d3)-2-((2S,4R)-2- (1-(methyl-d3)-1H-pyrazol-4- yl)tetrahydro-2H-pyran-4-yl)-4- (4-(methyl-d3)phenyl)pteridine | |
| 175 | 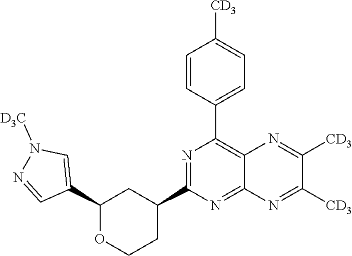 [SMILES: [2H]C([2H])([2H])c1ccc(-c2nc([C@H]3CCO[C@@H](c4cnn(C([2H])([2H])[2H])c4)C3)nc3nc(C([2H])([2H])[2H])c(C([2H])([2H])[2H])nc23)cc1] | 6,7-bis(methyl-d3)-2-((2R,4S)-2- (1-(methyl-d3)-1H-pyrazol-4- yl)tetrahydro-2H-pyran-4-yl)-4- (4-(methyl-d3)phenyl)pteridine | |
| 176 | 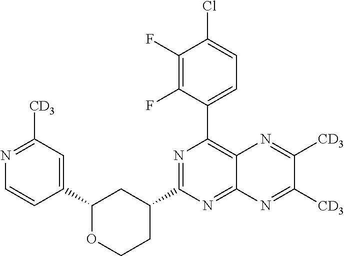 [SMILES: [2H]C([2H])([2H])c1cc([C@@H]2C[C@H](c3nc(-c4ccc(Cl)c(F)c4F)c4nc(C([2H])([2H])[2H])c(C([2H])([2H])[2H])nc4n3)CCO2)ccn1] | 4-(2,3-difluoro-4-(methyl- d3)phenyl)-6,7-bis(methyl-d3)- 2-((2S,4R)-2-(2-(methyl- d3)pyridin-4-yl)tetrahydro-2H- pyran-4-yl)pteridine | |
| 177 | 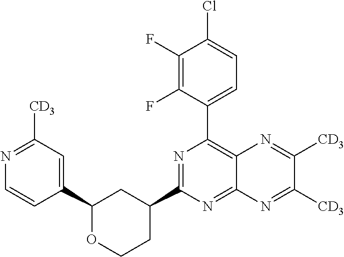 [SMILES: [2H]C([2H])([2H])c1cc([C@H]2C[C@@H](c3nc(-c4ccc(Cl)c(F)c4F)c4nc(C([2H])([2H])[2H])c(C([2H])([2H])[2H])nc4n3)CCO2)ccn1] | 4-(2,3-difluoro-4-(methyl- d3)phenyl)-6,7-bis(methyl-d3)- 2-((2R,4S)-2-(2-(methyl- d3)pyridin-4-yl)tetrahydro-2H- pyran-4-yl)pteridine | |
| 178 | 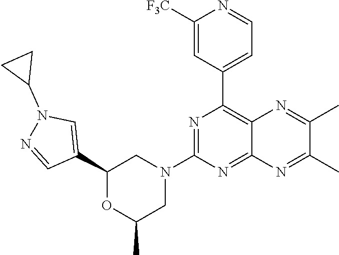 [SMILES: Cc1nc2nc(N3C[C@@H](C)O[C@@H](c4cnn(C5CC5)c4)C3)nc(-c3ccnc(C(F)(F)F)c3)c2nc1C] | 2S,6R)-2-(1-cyclopropyl-1H- pyrazol-4-yl)-4-(6,7-dimethyl-4- (2-(trifluoromethyl)pyridin-4- yl)pteridin-2-yl)-6- methylmorpholine | |
| 179 | 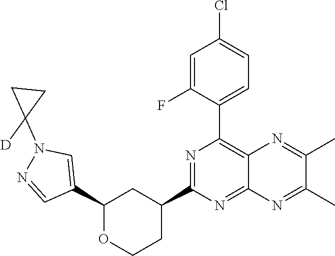 [SMILES: [2H]C1(n2cc([C@H]3C[C@@H](c4nc(-c5ccc(Cl)cc5F)c5nc(C)c(C)nc5n4)CCO3)cn2)CC1] | 4-(4-chloro-2-fluorophenyl)-2- ((2R,4S)-2-(1-(cyclopropyl-1-d)- 1H-pyrazol-4-yl)tetrahydro-2H- pyran-4-yl)-6,7- dimethylpteridine | |
| 180 | 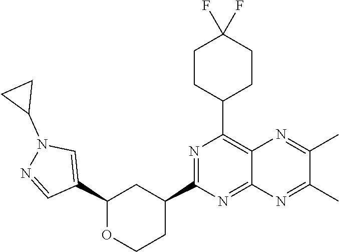 [SMILES: Cc1nc2nc([C@H]3CCO[C@@H](c4cnn(C5CC5)c4)C3)nc(C3CCC(F)(F)CC3)c2nc1C] | 2-((2R,4S)-2-(1-cyclopropyl-1H- pyrazol-4-yl)tetrahydro-2H- pyran-4-yl)-4-(4,4- difluorocyclohexyl)-6,7- dimethylpteridine | |
| 181 | 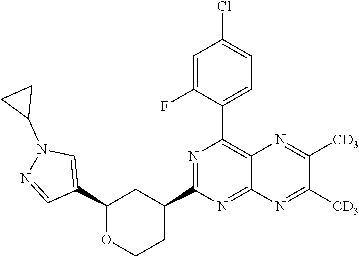 [SMILES: [2H]C([2H])([2H])c1nc2nc([C@H]3CCO[C@@H](c4cnn(C5CC5)c4)C3)nc(-c3ccc(Cl)cc3F)c2nc1C([2H])([2H])[2H]] | 4-(4-chloro-2-fluorophenyl)-2- ((2R,4S)-2-(1-cyclopropyl-1H- pyrazol-4-yl)tetrahydro-2H- pyran-4-yl)-6,7-bis(methyl- d3)pteridine | |
| 182 | 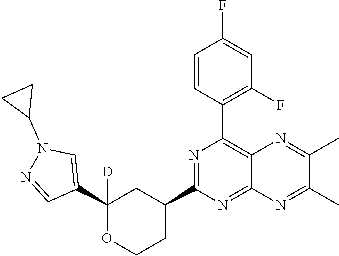 [SMILES: [2H][C@]1(c2cnn(C3CC3)c2)C[C@@H](c2nc(-c3ccc(F)cc3F)c3nc(C)c(C)nc3n2)CCO1] | 2-((2R,4S)-2-(1-cyclopropyl-1H- pyrazol-4-yl)tetrahydro-2H- pyran-4-yl-2-d)-4-(2,4- difluorophenyl)-6,7- dimethylpteridine | |
| 183 | 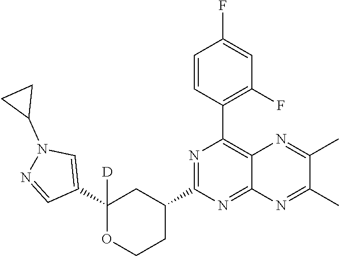 [SMILES: [2H][C@@]1(c2cnn(C3CC3)c2)C[C@H](c2nc(-c3ccc(F)cc3F)c3nc(C)c(C)nc3n2)CCO1] | 2-((2S,4R)-2-(1-cyclopropyl-1H- pyrazol-4-yl)tetrahydro-2H- pyran-4-yl-2-d)-4-(2,4- difluorophenyl)-6,7- dimethylpteridine | |
| 184 | 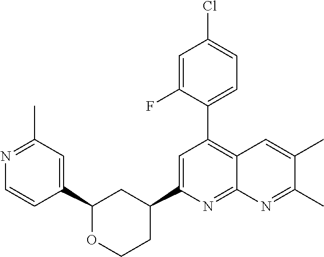 [SMILES: Cc1cc([C@H]2C[C@@H](c3cc(-c4ccc(Cl)cc4F)c4cc(C)c(C)nc4n3)CCO2)ccn1] | 5-(4-chloro-2-fluorophenyl)-2,3- dimethyl-7-((2R,4S)-2-(2- methylpyridin-4-yl)tetrahydro- 2H-pyran-4-yl)-1,8- naphthyridine | |
| 185 | 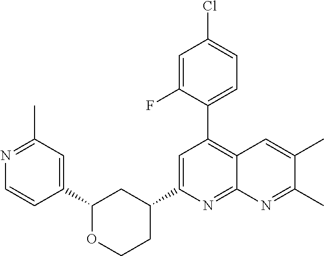 [SMILES: Cc1cc([C@@H]2C[C@H](c3cc(-c4ccc(Cl)cc4F)c4cc(C)c(C)nc4n3)CCO2)ccn1] | 5-(4-chloro-2-fluorophenyl)-2,3- dimethyl-7-((2S,4R)-2-(2- methylpyridin-4-yl)tetrahydro- 2H-pyran-4-yl)-1,8- naphthyridine | |
| 186 | 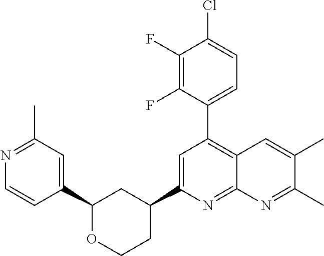 [SMILES: Cc1cc([C@H]2C[C@@H](c3cc(-c4ccc(Cl)c(F)c4F)c4cc(C)c(C)nc4n3)CCO2)ccn1] | 5-(4-chloro-2,3-difluorophenyl)- 2,3-dimethyl-7-((2R,4S)-2-(2- methylpyridin-4-yl)tetrahydro- 2H-pyran-4-yl)-1,8- naphthyridine | |
| 187 | 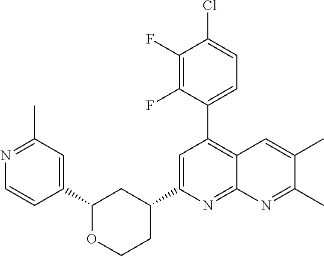 [SMILES: Cc1cc([C@@H]2C[C@H](c3cc(-c4ccc(Cl)c(F)c4F)c4cc(C)c(C)nc4n3)CCO2)ccn1] | 5-(4-chloro-2,3-difluorophenyl)- 2,3-dimethyl-7-((2S,4R)-2-(2- methylpyridin-4-yl)tetrahydro- 2H-pyran-4-yl)-1,8- naphthyridine | |
| 188 | 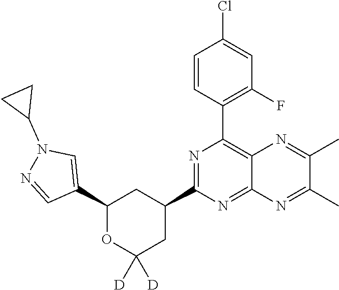 [SMILES: [2H]C1([2H])C[C@H](c2nc(-c3ccc(Cl)cc3F)c3nc(C)c(C)nc3n2)C[C@H](c2cnn(C3CC3)c2)O1] | 4-(4-chloro-2-fluorophenyl)-2- ((2R,4S)-2-(1-cyclopropyl-1H- pyrazol-4-yl)tetrahydro-2H- pyran-4-yl-6,6-d2)-6,7- dimethylpteridine | |
| 189 | 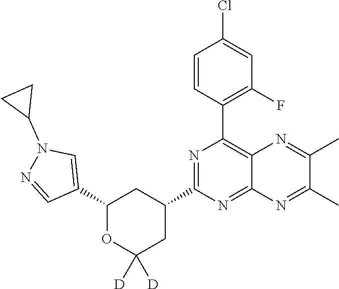 [SMILES: [2H]C1([2H])C[C@@H](c2nc(-c3ccc(Cl)cc3F)c3nc(C)c(C)nc3n2)C[C@@H](c2cnn(C3CC3)c2)O1] | 4-(4-chloro-2-fluorophenyl)-2- ((2S,4R)-2-(1-cyclopropyl-1H- pyrazol-4-yl)tetrahydro-2H- pyran-4-yl-6,6-d2)-6,7- dimethylpteridine | |
| 190 | 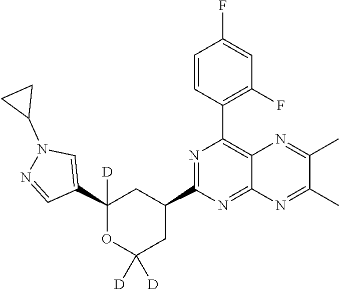 [SMILES: [2H]C1([2H])C[C@H](c2nc(-c3ccc(F)cc3F)c3nc(C)c(C)nc3n2)C[C@]([2H])(c2cnn(C3CC3)c2)O1] | 2-((2R,4S)-2-(1-cyclopropyl-1H- pyrazol-4-yl)tetrahydro-2H- pyran-4-yl-2,6,6-d3)-4-(2,4- difluorophenyl)-6,7- dimethylpteridine | |
| 191 | 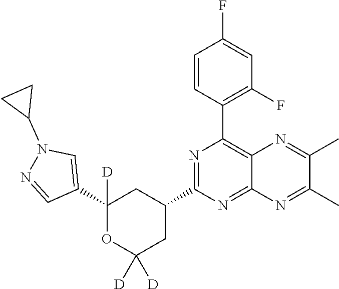 [SMILES: [2H]C1([2H])C[C@@H](c2nc(-c3ccc(F)cc3F)c3nc(C)c(C)nc3n2)C[C@@]([2H])(c2cnn(C3CC3)c2)O1] | 2-((2S,4R)-2-(1-cyclopropyl-1H- pyrazol-4-yl)tetrahydro-2H- pyran-4-yl-2,6,6-d3)-4-(2,4- difluorophenyl)-6,7- dimethylpteridine | |
| 192 | 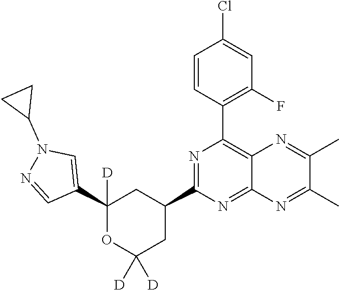 [SMILES: [2H]C1([2H])C[C@H](c2nc(-c3ccc(Cl)cc3F)c3nc(C)c(C)nc3n2)C[C@]([2H])(c2cnn(C3CC3)c2)O1] | 4-(4-chloro-2-fluorophenyl)-2- ((2R,4S)-2-(1-cyclopropyl-1H- pyrazol-4-yl)tetrahydro-2H- pyran-4-yl-2,6,6-d3)-6,7- dimethylpteridine | |
| 193 | 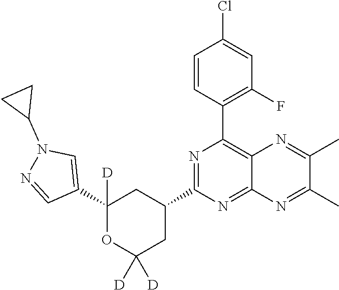 [SMILES: [2H]C1([2H])C[C@@H](c2nc(-c3ccc(Cl)cc3F)c3nc(C)c(C)nc3n2)C[C@@]([2H])(c2cnn(C3CC3)c2)O1] | 4-(4-chloro-2-fluorophenyl)-2- ((2S,4R)-2-(1-cyclopropyl-1H- pyrazol-4-yl)tetrahydro-2H- pyran-4-yl-2,6,6-d3)-6,7- dimethylpteridine | |
| 194 | 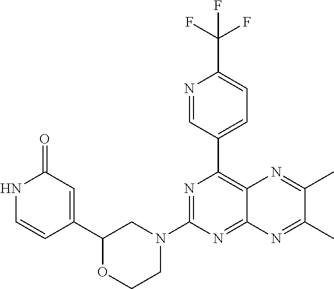 [SMILES: Cc1nc2nc(N3CCOC(c4cc[nH]c(=O)c4)C3)nc(-c3ccc(C(F)(F)F)nc3)c2nc1C] | 4-(4-(6,7-dimethyl-4-(6- (trifluoromethyl)pyridin-3- yl)pteridin-2-yl)morpholin-2- yl)pyridin-2(1H)-one | |
| 195 | 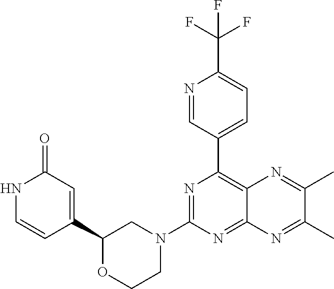 [SMILES: Cc1nc2nc(N3CCO[C@@H](c4cc[nH]c(=O)c4)C3)nc(-c3ccc(C(F)(F)F)nc3)c2nc1C] | (S)-4-(4-(6,7-dimethyl-4-(6- (trifluoromethyl)pyridin-3- yl)pteridin-2-yl)morpholin-2- yl)pyridin-2(1H)-one | |
| 196 | 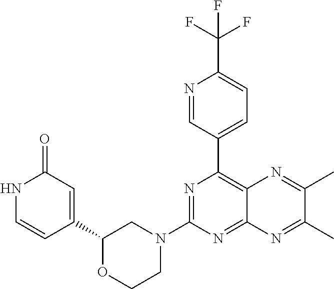 [SMILES: Cc1nc2nc(N3CCO[C@H](c4cc[nH]c(=O)c4)C3)nc(-c3ccc(C(F)(F)F)nc3)c2nc1C] | (R)-4-(4-(6,7-dimethyl-4-(6- (trifluoromethyl)pyridin-3- yl)pteridin-2-yl)morpholin-2- yl)pyridin-2(1H)-one | |
| 197 | 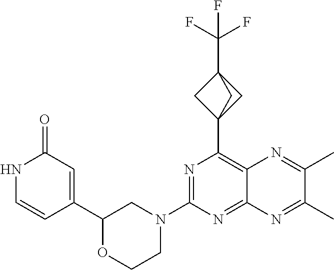 [SMILES: Cc1nc2nc(N3CCOC(c4cc[nH]c(=O)c4)C3)nc(C34CC(C(F)(F)F)(C3)C4)c2nc1C] | 4-(4-(6,7-dimethyl-4-(3- (trifluoromethyl)bicyclo[1.1.1] pentan-1-yl)pteridin-2- yl)morpholin-2-yl)pyridin- 2(1H)-one | |
| 198 | 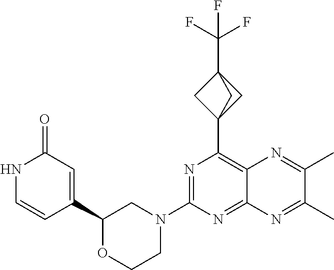 [SMILES: Cc1nc2nc(N3CCO[C@@H](c4cc[nH]c(=O)c4)C3)nc(C34CC(C(F)(F)F)(C3)C4)c2nc1C] | (S)-4-(4-(6,7-dimethyl-4-(3- (trifluoromethyl)bicyclo[1.1.1] pentan-1-yl)pteridin-2- yl)morpholin-2-yl)pyridin- 2(1H)-one | |
| 199 | 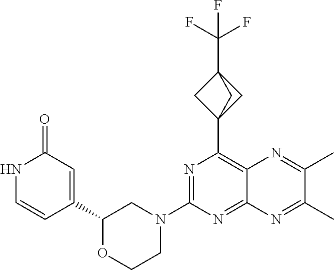 [SMILES: Cc1nc2nc(N3CCO[C@H](c4cc[nH]c(=O)c4)C3)nc(C34CC(C(F)(F)F)(C3)C4)c2nc1C] | (R)-4-(4-(6,7-dimethyl-4-(3- (trifluoromethyl)bicyclo[1.1.1] pentan-1-yl)pteridin-2- yl)morpholin-2-yl)pyridin- 2(1H)-one | |
| 200 | 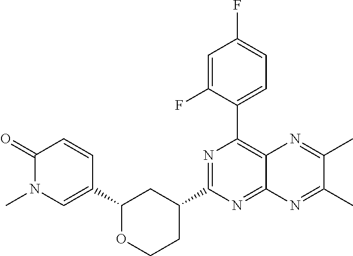 [SMILES: Cc1nc2nc([C@@H]3CCO[C@H](c4ccc(=O)n(C)c4)C3)nc(-c3ccc(F)cc3F)c2nc1C] | 5-((2S,4R)-4-(4-(2-fluoro-4- (trifluoromethyl)phenyl)-6,7- dimethylpteridin-2- yl)tetrahydro-2H-pyran-2-yl)-1- methylpyridin-2(1H)-one | Method 16 |
| 201 | 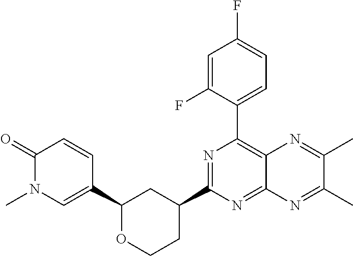 [SMILES: Cc1nc2nc([C@H]3CCO[C@@H](c4ccc(=O)n(C)c4)C3)nc(-c3ccc(F)cc3F)c2nc1C] | 5-((2R,4S)-4-(4-(2-fluoro-4- (trifluoromethyl)phenyl)-6,7- dimethylpteridin-2- yl)tetrahydro-2H-pyran-2-yl)-1- methylpyridin-2(1H)-one | Method 16 |
| 202 | 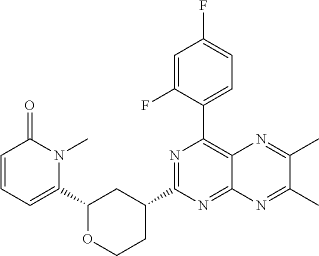 [SMILES: Cc1nc2nc([C@@H]3CCO[C@H](c4cccc(=O)n4C)C3)nc(-c3ccc(F)cc3F)c2nc1C] | 6-((2S,4R)-4-(4-(2-fluoro-4- (trifluoromethyl)phenyl)-6,7- dimethylpteridin-2- yl)tetrahydro-2H-pyran-2-yl)-1- methylpyridin-2(1H)-one | Method 17 |
| 203 | 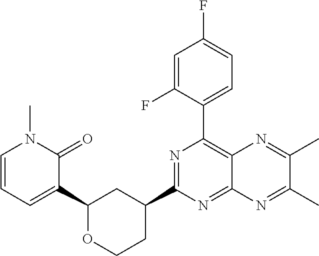 [SMILES: Cc1nc2nc([C@H]3CCO[C@@H](c4cccn(C)c4=O)C3)nc(-c3ccc(F)cc3F)c2nc1C] | 3-((2R,4S)-4-(4-(2-fluoro-4- (trifluoromethyl)phenyl)-6,7- dimethylpteridin-2- yl)tetrahydro-2H-pyran-2-yl)-1- methylpyridin-2(1H)-one | Method 17 |
| 204 | 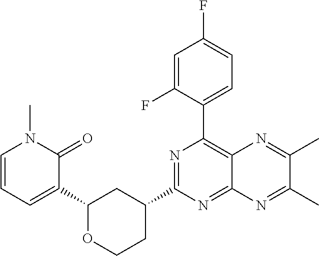 [SMILES: Cc1nc2nc([C@@H]3CCO[C@H](c4cccn(C)c4=O)C3)nc(-c3ccc(F)cc3F)c2nc1C] | 3-((2S,4R)-4-(4-(2-fluoro-4- (trifluoromethyl)phenyl)-6,7- dimethylpteridin-2- yl)tetrahydro-2H-pyran-2-yl)-1- methylpyridin-2(1H)-one | Method 17 |
| 205 | 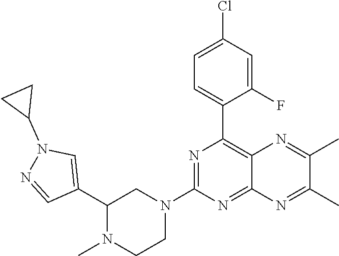 [SMILES: Cc1nc2nc(N3CCN(C)C(c4cnn(C5CC5)c4)C3)nc(-c3ccc(Cl)cc3F)c2nc1C] | 4-(4-chloro-2-fluorophenyl)-2- (3-(1-cyclopropyl-1H-pyrazol-4- yl)-4-methylpiperazin-1-yl)-6,7- dimethylpteridine | |
| 206 | 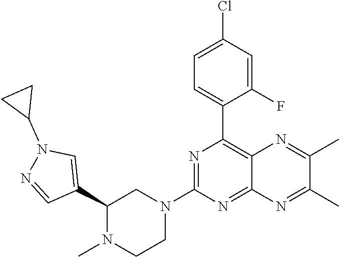 [SMILES: Cc1nc2nc(N3CCN(C)[C@@H](c4cnn(C5CC5)c4)C3)nc(-c3ccc(Cl)cc3F)c2nc1C] | (S)-4-(4-chloro-2-fluorophenyl)- 2-(3-(1-cyclopropyl-1H-pyrazol- 4-yl)-4-methylpiperazin-1-yl)- 6,7-dimethylpteridine | |
| 207 | 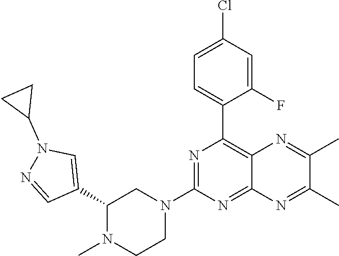 [SMILES: Cc1nc2nc(N3CCN(C)[C@H](c4cnn(C5CC5)c4)C3)nc(-c3ccc(Cl)cc3F)c2nc1C] | (R)-4-(4-chloro-2-fluorophenyl)- 2-(3-(1-cyclopropyl-1H-pyrazol- 4-yl)-4-methylpiperazin-1-yl)- 6,7-dimethylpteridine | |
| 208 | 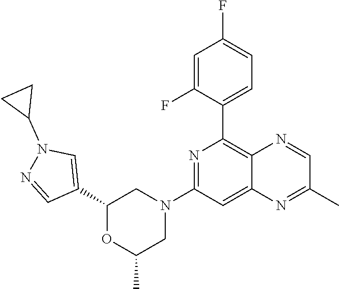 [SMILES: Cc1cnc2c(-c3ccc(F)cc3F)nc(N3C[C@@H](c4cnn(C5CC5)c4)O[C@@H](C)C3)cc2n1] | (2R,6S)-2-(1-cyclopropyl-1H- pyrazol-4-yl)-4-(5-(2,4- difluorophenyl)-2- methylpyrido[3,4-b]pyrazin-7- yl)-6-methylmorpholine | Method 5 |
| 209 | 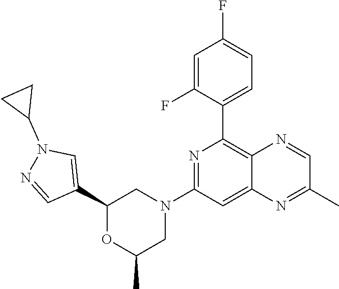 [SMILES: Cc1cnc2c(-c3ccc(F)cc3F)nc(N3C[C@@H](C)O[C@@H](c4cnn(C5CC5)c4)C3)cc2n1] | (2S,6R)-2-(1-cyclopropyl-1H- pyrazol-4-yl)-4-(5-(2,4- difluorophenyl)-2- methylpyrido[3,4-b]pyrazin-7- yl)-6-methylmorpholine | Method 5 |
| 210 | 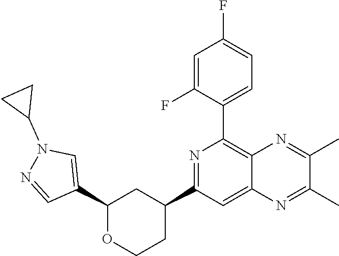 [SMILES: Cc1nc2cc([C@H]3CCO[C@@H](c4cnn(C5CC5)c4)C3)nc(-c3ccc(F)cc3F)c2nc1C] | 7-((2R,4S)-2-(1-cyclopropyl-1H- pyrazol-4-yl)tetrahydro-2H- pyran-4-yl)-5-(2,4- difluorophenyl)-2,3- dimethylpyrido[3,4-b]pyrazine | Method 11 |
| 211 | 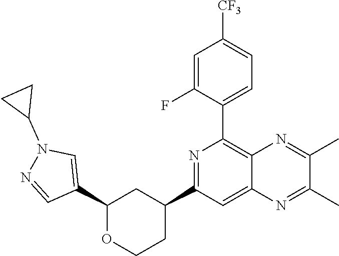 [SMILES: Cc1nc2cc([C@H]3CCO[C@@H](c4cnn(C5CC5)c4)C3)nc(-c3ccc(C(F)(F)F)cc3F)c2nc1C] | 7-((2R,4S)-2-(1-cyclopropyl-1H- pyrazol-4-yl)tetrahydro-2H- pyran-4-yl)-5-(2-fluoro-4- (trifluoromethyl)phenyl)-2,3- dimethylpyrido[3,4-b]pyrazine | Method 11 |
| 212 | 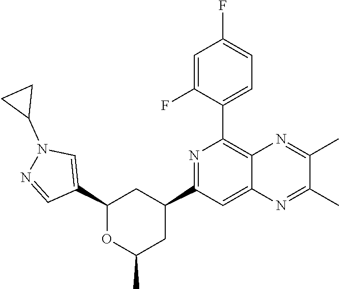 [SMILES: Cc1nc2cc([C@H]3C[C@@H](C)O[C@@H](c4cnn(C5CC5)c4)C3)nc(-c3ccc(F)cc3F)c2nc1C] | 7-((2R,4S,6R)-2-(1-cyclopropyl- 1H-pyrazol-4-yl)-6- methyltetrahydro-2H-pyran-4- yl)-5-(2,4-difluorophenyl)-2,3- dimethylpyrido[3,4-b]pyrazine | |
| 213 | 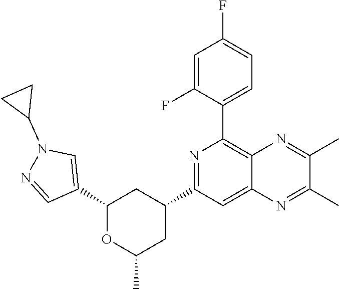 [SMILES: Cc1nc2cc([C@H]3C[C@@H](c4cnn(C5CC5)c4)O[C@@H](C)C3)nc(-c3ccc(F)cc3F)c2nc1C] | 7-((2S,4R,6S)-2-(1-cyclopropyl- 1H-pyrazol-4-yl)-6- methyltetrahydro-2H-pyran-4- yl)-5-(2,4-difluorophenyl)-2,3- dimethylpyrido[3,4-b]pyrazine | |
| 214 | 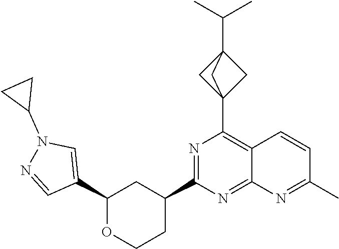 [SMILES: Cc1ccc2c(C34CC(C(C)C)(C3)C4)nc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-4-(3- isopropylbicyclo[1.1.1]pentan-1- yl)-7-methylpyrido[2,3- d]pyrimidine | |
| 215 | 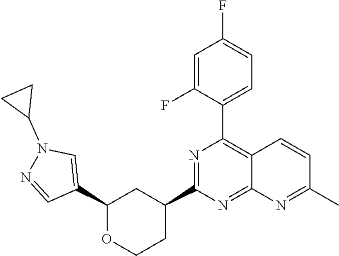 [SMILES: Cc1ccc2c(-c3ccc(F)cc3F)nc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-4-(2,4- difluorophenyl)-7- methylpyrido[2,3-d]pyrimidine | |
| 216 | 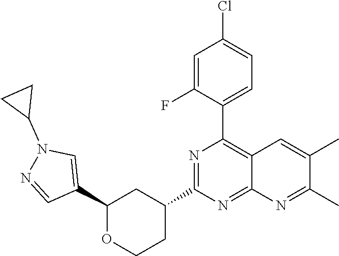 [SMILES: Cc1cc2c(-c3ccc(Cl)cc3F)nc([C@@H]3CCO[C@@H](c4cnn(C5CC5)c4)C3)nc2nc1C] | 4-(4-chloro-2-fluorophenyl)-2- ((2R,4R)-2-(1-cyclopropyl-1H- pyrazol-4-yl)tetrahydro-2H- pyran-4-yl)-6,7- dimethylpyrido[2,3-d]pyrimidine | Method 2 |
| 217 | 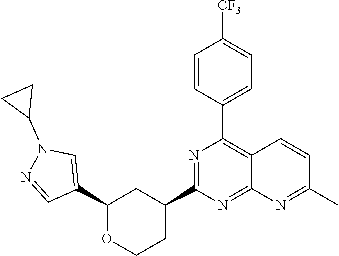 [SMILES: Cc1ccc2c(-c3ccc(C(F)(F)F)cc3)nc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-7-methyl-4-(4- (trifluoromethyl)phenyl)pyrido [2,3-d]pyrimidine | |
| 218 | 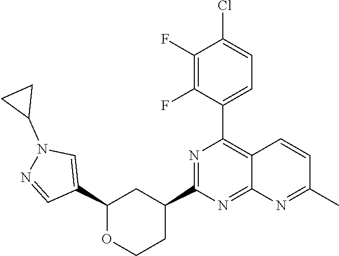 [SMILES: Cc1ccc2c(-c3ccc(Cl)c(F)c3F)nc([C@H]3CCO[C@@H](c4cnn(C5CC5)c4)C3)nc2n1] | 4-(4-chloro-2,3-difluorophenyl)- 2-((2R,4S)-2-(1-cyclopropyl-1H- pyrazol-4-yl)tetrahydro-2H- pyran-4-yl)-7-methylpyrido[2,3- d]pyrimidine | |
| 219 | 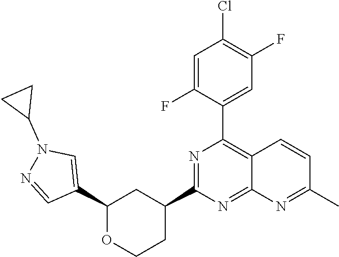 [SMILES: Cc1ccc2c(-c3cc(F)c(Cl)cc3F)nc([C@H]3CCO[C@@H](c4cnn(C5CC5)c4)C3)nc2n1] | 4-(4-chloro-2,5-difluorophenyl)- 2-((2R,4S)-2-(1-cyclopropyl-1H- pyrazol-4-yl)tetrahydro-2H- pyran-4-yl)-7-methylpyrido[2,3- d]pyrimidine | |
| 220 | 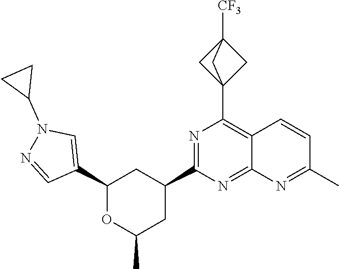 [SMILES: Cc1ccc2c(C34CC(C(F)(F)F)(C3)C4)nc([C@H]3C[C@@H](C)O[C@@H](c4cnn(C5CC5)c4)C3)nc2n1] | 2-((2R,4S,6R)-2-(1-cyclopropyl- 1H-pyrazol-4-yl)-6- methyltetrahydro-2H-pyran-4- yl)-7-methyl-4-(3- (trifluoromethyl)bicyclo[1.1.1] pentan-1-yl)pyrido[2,3- d]pyrimidine | |
| 221 | 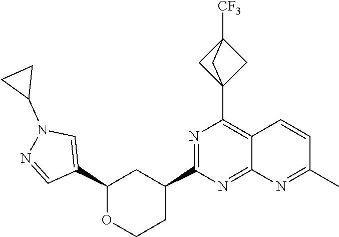 [SMILES: Cc1ccc2c(C34CC(C(F)(F)F)(C3)C4)nc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-7-methyl-4-(3- (trifluoromethyl)bicyclo[1.1.1] pentan-1-yl)pyrido[2,3- d]pyrimidine | |
| 222 | 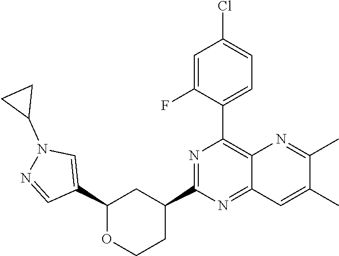 [SMILES: Cc1cc2nc([C@H]3CCO[C@@H](c4cnn(C5CC5)c4)C3)nc(-c3ccc(Cl)cc3F)c2nc1C] | 4-(4-chloro-2-fluorophenyl)-2- ((2R,4S)-2-(1-cyclopropyl-1H- pyrazol-4-yl)tetrahydro-2H- pyran-4-yl)-6,7- dimethylpyrido[3,2-d]pyrimidine | Method 11 |
| 223 | 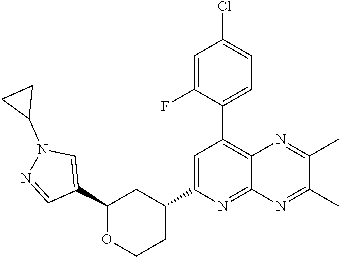 [SMILES: Cc1nc2nc([C@@H]3CCO[C@@H](c4cnn(C5CC5)c4)C3)cc(-c3ccc(Cl)cc3F)c2nc1C] | 8-(4-chloro-2-fluorophenyl)-6- ((2R,4R)-2-(1-cyclopropyl-1H- pyrazol-4-yl)tetrahydro-2H- pyran-4-yl)-2,3- dimethylpyrido[2,3-b]pyrazine | Method 10 |
| 224 | 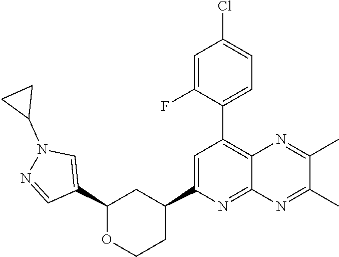 [SMILES: Cc1nc2nc([C@H]3CCO[C@@H](c4cnn(C5CC5)c4)C3)cc(-c3ccc(Cl)cc3F)c2nc1C] | 8-(4-chloro-2-fluorophenyl)-6- ((2R,4S)-2-(1-cyclopropyl-1H- pyrazol-4-yl)tetrahydro-2H- pyran-4-yl)-2,3- dimethylpyrido[2,3-b]pyrazine | Method 10 |
| 225 | 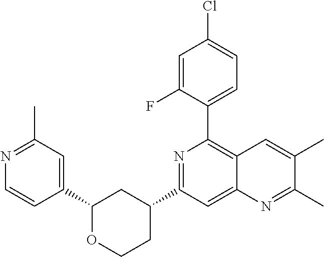 [SMILES: Cc1cc([C@@H]2C[C@H](c3cc4nc(C)c(C)cc4c(-c4ccc(Cl)cc4F)n3)CCO2)ccn1] | 5-(4-chloro-2-fluorophenyl)-2,3- dimethyl-7-((2S,4R)-2-(2- methylpyridin-4-yl)tetrahydro- 2H-pyran-4-yl)-1,6- naphthyridine | |
| 226 | 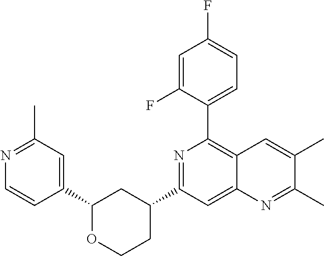 [SMILES: Cc1cc([C@@H]2C[C@H](c3cc4nc(C)c(C)cc4c(-c4ccc(F)cc4F)n3)CCO2)ccn1] | 5-(2,4-difluorophenyl)-2,3- dimethyl-7-((2S,4R)-2-(2- methylpyridin-4-yl)tetrahydro- 2H-pyran-4-yl)-1,6- naphthyridine | |
| 227 | 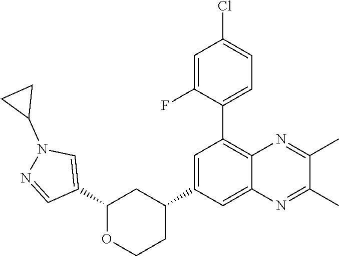 [SMILES: Cc1nc2cc([C@@H]3CCO[C@H](c4cnn(C5CC5)c4)C3)cc(-c3ccc(Cl)cc3F)c2nc1C] | 5-(4-chloro-2-fluorophenyl)-7- ((2S,4R)-2-(1-cyclopropyl-1H- pyrazol-4-yl)tetrahydro-2H- pyran-4-yl)-2,3- dimethylquinoxaline | Method 8 |
| 228 | 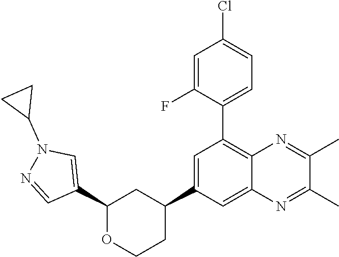 [SMILES: Cc1nc2cc([C@H]3CCO[C@@H](c4cnn(C5CC5)c4)C3)cc(-c3ccc(Cl)cc3F)c2nc1C] | 5-(4-chloro-2-fluorophenyl)-7- ((2R,4S)-2-(1-cyclopropyl-1H- pyrazol-4-yl)tetrahydro-2H- pyran-4-yl)-2,3- dimethylquinoxaline | Method 8 |
| 229 | 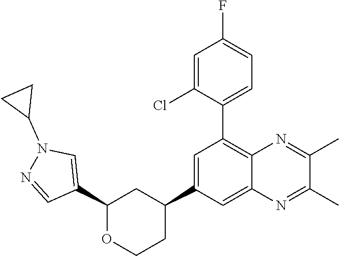 [SMILES: Cc1nc2cc([C@H]3CCO[C@@H](c4cnn(C5CC5)c4)C3)cc(-c3ccc(F)cc3Cl)c2nc1C] | 5-(2-chloro-4-fluorophenyl)-7- ((2R,4S)-2-(1-cyclopropyl-1H- pyrazol-4-yl)tetrahydro-2H- pyran-4-yl)-2,3- dimethylquinoxaline | |
| 230 | 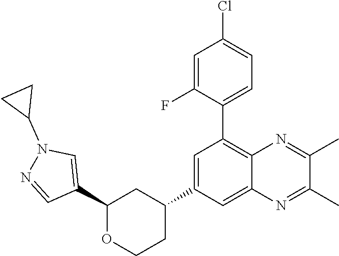 [SMILES: Cc1nc2cc([C@@H]3CCO[C@@H](c4cnn(C5CC5)c4)C3)cc(-c3ccc(Cl)cc3F)c2nc1C] | 5-(4-chloro-2-fluorophenyl)-7- ((2R,4R)-2-(1-cyclopropyl-1H- pyrazol-4-yl)tetrahydro-2H- pyran-4-yl)-2,3- dimethylquinoxaline | |
| 231 | 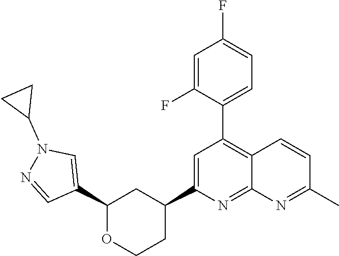 [SMILES: Cc1ccc2c(-c3ccc(F)cc3F)cc([C@H]3CCO[C@@H](c4cnn(C5CC5)c4)C3)nc2n1] | 2-((2R,4S)-2-(1-cyclopropyl-1H- pyrazol-4-yl)tetrahydro-2H- pyran-4-yl)-4-(2-fluoro-4- (trifluoromethyl)phenyl)-7- methyl-1,8-naphthyridine | |
| 232 | 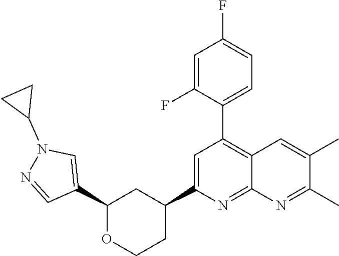 [SMILES: Cc1cc2c(-c3ccc(F)cc3F)cc([C@H]3CCO[C@@H](c4cnn(C5CC5)c4)C3)nc2nc1C] | 7-((2R,4S)-2-(1-cyclopropyl-1H- pyrazol-4-yl)tetrahydro-2H- pyran-4-yl)-5-(2-fluoro-4- (trifluoromethyl)phenyl)-2,3- dimethyl-1,8-naphthyridine | |
| 233 | 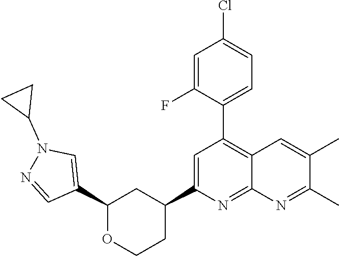 [SMILES: Cc1cc2c(-c3ccc(Cl)cc3F)cc([C@H]3CCO[C@@H](c4cnn(C5CC5)c4)C3)nc2nc1C] | 5-(4-chloro-2-fluoro-phenyl)-7- [(2R,4S)-2-(1- cyclopropylpyrazol-4- yl)tetrahydropyran-4-yl]-2,3- dimethyl-1,8-naphthyridine | |
| 234 | 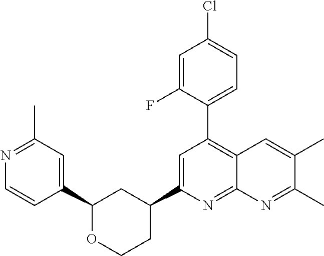 [SMILES: Cc1cc([C@H]2C[C@@H](c3cc(-c4ccc(Cl)cc4F)c4cc(C)c(C)nc4n3)CCO2)ccn1] | 5-(4-chloro-2-fluorophenyl)-2,3- dimethyl-7-((2R,4S)-2-(2- methylpyridin-4-yl)tetrahydro- 2H-pyran-4-yl)-1,8- naphthyridine | |
| 235 | 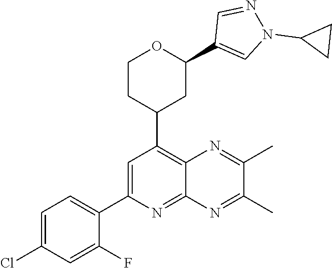 | 6-(4-chloro-2-fluorophenyl)-8- ((2R)-2-(1-cyclopropyl-1H- pyrazol-4-yl)tetrahydro-2H- pyran-4-yl)-2,3- dimethylpyrido[2,3-b]pyrazine | Method 10, byproduct of synthesis of Ex.s223/224 |
| 236 | 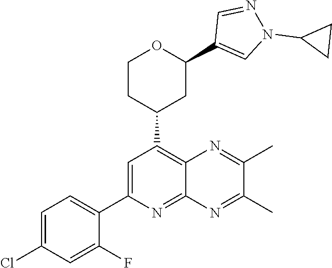 [SMILES: Cc1nc2nc(-c3ccc(Cl)cc3F)cc([C@@H]3CCO[C@@H](c4cnn(C5CC5)c4)C3)c2nc1C] | 6-(4-chloro-2-fluorophenyl)-8- ((2R,4R)-2-(1-cyclopropyl-1H- pyrazol-4-yl)tetrahydro-2H- pyran-4-yl)-2,3- dimethylpyrido[2,3-b]pyrazine | Method 10, byproduct of synthesis of Ex.s223/224 |
| 237 | 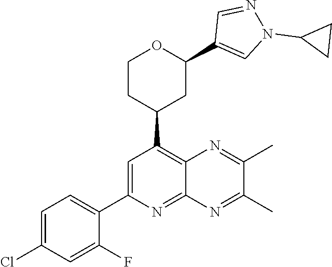 [SMILES: Cc1nc2nc(-c3ccc(Cl)cc3F)cc([C@H]3CCO[C@@H](c4cnn(C5CC5)c4)C3)c2nc1C] | 6-(4-chloro-2-fluorophenyl)-8- ((2R,4S)-2-(1-cyclopropyl-1H- pyrazol-4-yl)tetrahydro-2H- pyran-4-yl)-2,3- dimethylpyrido[2,3-b]pyrazine | Method 10, byproduct of synthesis of Ex.s223/224 |
| 238 | 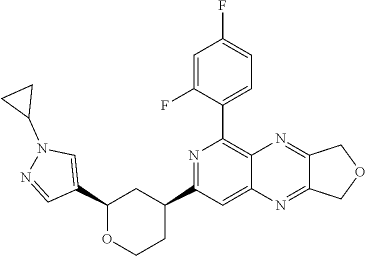 [SMILES: Fc1ccc(-c2nc([C@H]3CCO[C@@H](c4cnn(C5CC5)c4)C3)cc3nc4c(nc23)COC4)c(F)c1] | 7-((2R,4S)-2-(1-cyclopropyl-1H- pyrazol-4-yl)tetrahydro-2H- pyran-4-yl)-5-(2,4- difluorophenyl)-1,3- dihydrofuro[3,4-b]pyrido[3,4- e]pyrazine | |
| 239 | 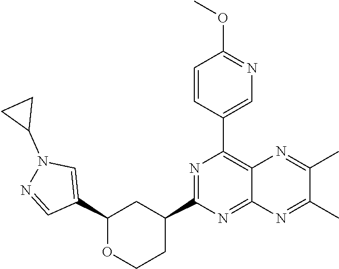 [SMILES: COc1ccc(-c2nc([C@H]3CCO[C@@H](c4cnn(C5CC5)c4)C3)nc3nc(C)c(C)nc23)cn1] | 2-((2R,4S)-2-(1-cyclopropyl-1H- pyrazol-4-yl)tetrahydro-2H- pyran-4-yl)-4-(6- methoxypyridin-3-yl)-6,7- dimethylpteridine | |
| 240 | 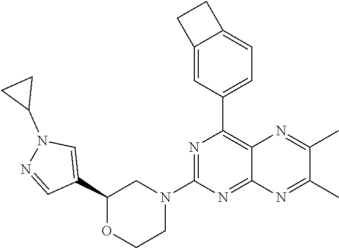 [SMILES: Cc1nc2nc(N3CCO[C@@H](c4cnn(C5CC5)c4)C3)nc(-c3ccc4c(c3)CC4)c2nc1C] | (S)-4-(4-(bicyclo[4.2.0]octa- 1,3,5-trien-3-yl)-6,7- dimethylpteridin-2-yl)-2-(1- cyclopropyl-1H-pyrazol-4- yl)morpholine | |
| 241 | 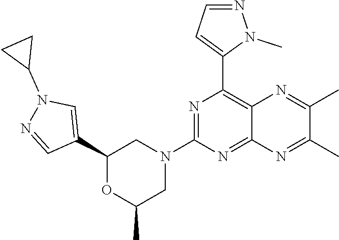 [SMILES: Cc1nc2nc(N3C[C@@H](C)O[C@@H](c4cnn(C5CC5)c4)C3)nc(-c3ccnn3C)c2nc1C] | (2S,6R)-2-(1-cyclopropyl-1H- pyrazol-4-yl)-4-(6,7-dimethyl-4- (1-methyl-1H-pyrazol-5- yl)pteridin-2-yl)-6- methylmorpholine | |
| 242 | 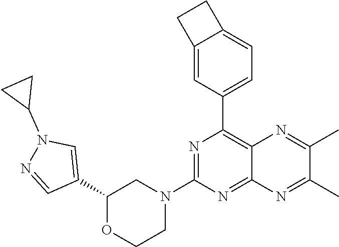 [SMILES: Cc1nc2nc(N3CCO[C@H](c4cnn(C5CC5)c4)C3)nc(-c3ccc4c(c3)CC4)c2nc1C] | (R)-4-(4-(bicyclo[ 4.2.0]octa- 1,3,5-trien-3-yl)-6,7- dimethylpteridin-2-yl)-2-(1- cyclopropyl-1H-pyrazol-4- yl)morpholine | |
| 243 | 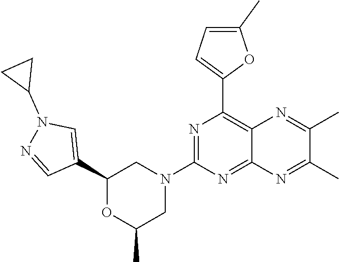 [SMILES: Cc1ccc(-c2nc(N3C[C@@H](C)O[C@@H](c4cnn(C5CC5)c4)C3)nc3nc(C)c(C)nc23)o1] | (2S,6R)-2-(1-cyclopropyl-1H- pyrazol-4-yl)-4-(6,7-dimethyl-4- (5-methylfuran-2-yl)pteridin-2- yl)-6-methylmorpholine | |
| 244 | 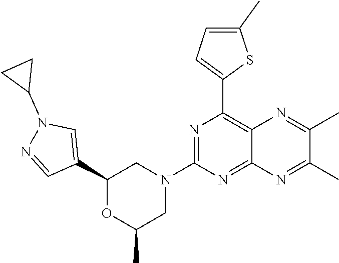 [SMILES: Cc1ccc(-c2nc(N3C[C@@H](C)O[C@@H](c4cnn(C5CC5)c4)C3)nc3nc(C)c(C)nc23)s1] | (2S,6R)-2-(1-cyclopropyl-1H- pyrazol-4-yl)-4-(6,7-dimethyl-4- (5-methylthiophen-2-yl)pteridin- 2-yl)-6-methylmorpholine | |
| 245 | 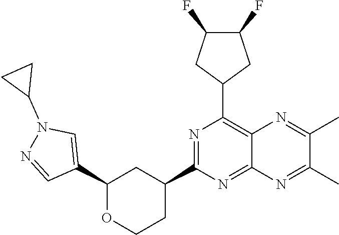 [SMILES: Cc1nc2nc([C@H]3CCO[C@@H](c4cnn(C5CC5)c4)C3)nc(C3C[C@@H](F)[C@@H](F)C3)c2nc1C] | 2-((2R,4S)-2-(1-cyclopropyl-1H- pyrazol-4-yl)tetrahydro-2H- pyran-4-yl)-4-((3R,4S)-3,4- difluorocyclopentyl)-6,7- dimethylpteridine | |
| 246 | 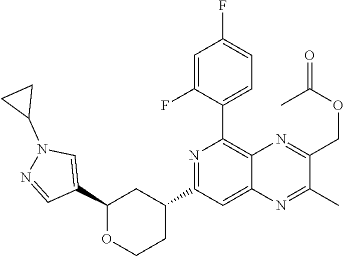 [SMILES: CC(=O)OCc1nc2c(-c3ccc(F)cc3F)nc([C@@H]3CCO[C@@H](c4cnn(C5CC5)c4)C3)cc2nc1C] | (7-((2R,4R)-2-(1-cyclopropyl- 1H-pyrazol-4-yl)tetrahydro-2H- pyran-4-yl)-5-(2,4- difluorophenyl)-2- methylpyrido[3,4-b]pyrazin-3- yl)methylacetate | |
| 247 | 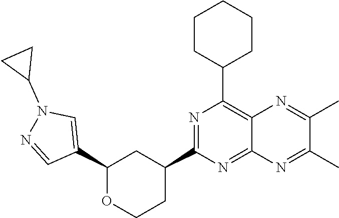 [SMILES: Cc1nc2nc([C@H]3CCO[C@@H](c4cnn(C5CC5)c4)C3)nc(C3CCCCC3)c2nc1C] | 4-cyclohexyl-2-((2R,4S)-2-(1- cyclopropyl-1H-pyrazol-4- yl)tetrahydro-2H-pyran-4-yl)- 6,7-dimethylpteridine | |
| 248 | 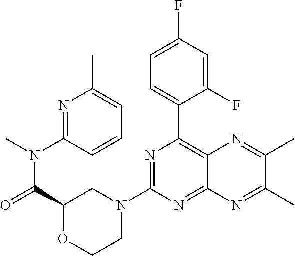 [SMILES: Cc1cccc(N(C)C(=O)[C@H]2CN(c3nc(-c4ccc(F)cc4F)c4nc(C)c(C)nc4n3)CCO2)n1] | (R)-4-(4-(2,4-difluorophenyl)- 6,7-dimethylpteridin-2-yl)-N- methyl-N-(6-methylpyridin-2- yl)morpholine-2-carboxamide | |

TABLE C
Analytical data for compounds of Table B

| Ex # | NMR | M + H |
|---|---|---|
| 1 | ¹H NMR (400 MHz, DMSO-d6) δ ppm 8.87 (1H, s), 8.39 (1H, d, J = 5.2 Hz), | 480.2 |
| | 7.85 (1H, s), 7.66 (1H, q, J = 7.7 Hz), 7.37-7.32 (1H, m), 7.31-7.29 (1H, m), | |
| | 7.26-7.20 (2H, m), 4.59 (1H, d, J = 11.1 Hz), 4.24-4.21 (1H, m), 3.80-3.73 | |
| | (1H, m), 2.73 (3H, s), 2.45 (3H, s), 2.28 (1H, d, J = 13.6 Hz), 2.00-1.96 (2H, | |
| | m), 1.72 (1H, q, J = 12.1 Hz). | |
| 2 | ¹H NMR (400 MHz, DMSO-d6) δ ppm 9.43 (1H, s), 9.00 (1H, s), 8.75 (1H, d, | 433.2 |
| | J = 8.2 Hz), 8.42 (1H, d, J = 5.2 Hz), 8.14-8.10 (2H, m), 7.94 (1H, s), 7.34 | |
| | (2H, s), 7.26 (2H, d, J = 5.1 Hz), 4.65 (1H, d, J = 11.1 Hz), 4.28 (1H, d, J = | |
| | 11.3 Hz), 3.89-3.79 (2H, m), 3.53 (2H, br s), 3.34 (3H, s), 2.80 (3H, s), 2.49 | |
| | (4H, s), 2.36 (2H, d, J = 12.8 Hz), 2.07-2.05 (2H, m), 1.82 (1H, q, J = 12.1 Hz). | |
| 3 | ¹H NMR (400 MHz, DMSO-d6) δ ppm 9.59 (1H, s), 8.90 (1H, d, J = 8.2 Hz), | 466.2 |
| | 8.37 (1H, d, J = 5.3 Hz), 8.15 (1H, d, J = 8.3 Hz), 7.27 (1H, s), 7.19 (1H, d, J = | |
| | 5.3 Hz), 4.62 (1H, d, J = 11.2 Hz), 4.25 (1H, dd, J = 11.3, 4.2 Hz), 3.80 (1H, | |
| | t, J = 11.8 Hz), 3.59 (1H, t, J = 11.6 Hz), 2.78 (3H, s), 2.73 (3H, s), 2.44 (4H, | |
| | s), 2.16 (1H, d, J = 13.1 Hz), 2.06-1.93 (1H, m), 1.77 (1H, q, J = 12.2 Hz). | |
| 4 | ¹H NMR (400 MHz, DMSO-d6): δ 9.59 (s, 1H), 8.90 (d, 1H), 8.37 (d, | 481.2 |
| | 1H), 8.15 (d, 1H), 7.27 (s, 1H), 7.19 (d, 1H), 4.62 (d, 1H), 4.25 (d, 1H), | |
| | 3.80 (t, 1H), 3.59 (s, 1H), 2.78 (s, 3H), 2.73 (s, 3H), 2.39-2.44 (m, 4H), | |
| | 2.16 (d, 1H), 1.99 (d, 1H), 1.77 (q, 1H). | |
| 5 | ¹H NMR (400 MHz, Chloroform-d): δH ppm 8.78 (1H, s), 8.74 (3H, s), 8.46 | 449.1 |
| | (4H, t, J = 5.5 Hz), 7.86 (1H, s), 7.74 (3H, s), 7.64 (1H, t, J = 7.9 Hz), 7.58 | |
| | (3H, t, J = 7.8 Hz), 7.33 (5H, t, J = 8.4 Hz), 7.22 (5H, s), 7.11 (5H, d, J = 5.0 | |
| | Hz), 4.91-4.88 (1H, m), 4.55 (4H, d, J = 11.2 Hz), 4.38 (4H, d, J = 11.5 Hz), | |
| | 3.96 (2H, dd, J = 6.4, 4.3 Hz), 3.87-3.81 (3H, m), 3.55-3.52 (1H, m), | |
| | 3.47-3.39 (3H, m), 2.80 (10H, s), 2.56-2.55 (14H, m), 2.33-2.24 (3H, m), | |
| | 2.15-2.07 (7H, m), 1.84 (4H, q, J = 12.2 Hz). | |
| 6 | ¹H NMR (400 MHz, Chloroform-d) δ ppm 9.60 (1H, s), 8.75 (1H, d, J = 8.2 | 446.8 |
| | Hz), 8.53 (1H, d, J = 5.1 Hz), 7.83 (1H, d, J = 8.1 Hz), 7.18 (1H, d, J = 5.1 | |
| | Hz), 7.03 (1H, s), 4.65 (1H, d, J = 10.5 Hz), 4.52 (1H, d, J = 13.0 Hz), 4.28 | |
| | (2H, t, J = 9.1 Hz), 3.94 (1H, dd, J = 12.7, 10.5 Hz), 3.23 (1H, dd, J = 13.3, | |
| | 10.4 Hz), 2.95 (1H, dd, J = 12.7, 10.6 Hz), 2.71 (3H, s), 2.67 (3H, s), 2.60 | |
| | (3H, s). Note: One aromatic proton is obscured by solvent signal. 19F NMR | |
| | (376 MHz, Chloroform-d) δ ppm-67.9. | |
| 7 | ¹H NMR (400 MHz, Chloroform-d) δ ppm 8.51 (d, J = 4.9 Hz, 1H), 8.46 (s, | 480.4 |
| | 1H), 7.66-7.58 (m, 1H), 7.18 (d, J = 5.5 Hz, 1H), 7.07-7.01 (m, 1H), | |
| | 7.00-6.93 (m, 2H), 4.64 (d, J = 9.9 Hz, 1H), 4.53 (d, J = 12.6 Hz, 1H), | |
| | 4.32-4.20 (m, 2H), 3.97-3.88 (m, 1H), 3.23 (td, J = 12.7, 3.3 Hz, 1H), | |
| | 3.00-2.89 (m, 1H), 2.71 (s, 3H), 2.59 (s, 3H). Note: One aromatic proton | |
| | is obscured by solvent signal. 19F NMR (376 MHz, Chloroform-d) | |
| | δ ppm-108.30 (s), -108.66 (s). | |
| 8 | ¹H NMR (400 MHz, CD2Cl2) δ ppm 8.41 (d, J = 5.1 Hz, 1H), 7.51-7.44 (m, | 434.8 |
| | 1H), 7.44-7.36 (m, 1H), 7.22 (s, 1H), 7.13 (d, J = 4.7 Hz, 1H), 4.54 (dd, J = | |
| | 11.6, 1.2 Hz, 1H), 4.37-4.30 (m, 1H), 3.88-3.77 (m, 1H), 3.63-3.52 (m, | |
| | 1H), 2.80 (s, 3H), 2.70 (s, 3H), 2.51 (s, 3H), 2.46-2.39 (m, 1H), 2.23-2.11 | |
| | (m, 2H), 2.00-1.89 (m, 1H) | |
| 9 | ¹H NMR (400 MHz, DMSO-d6) δ ppm 8.18 (d, J = 5.3 Hz, 1H), 7.74 (t, J = | 481.2 |
| | 7.9 Hz, 1H), 7.65 (d, J = 9.7 Hz, 1H), 7.50 (d, J = 8.3 Hz, 1H), 7.06 (d, J = 5.3 | |
| | Hz, 1H), 6.87 (s, 1H), 4.81 (d, J = 13.2 Hz, 1H), 4.66 (t, J = 12.5 Hz, 2H), | |
| | 4.14 (d, J = 11.5 Hz, 1H), 3.86 (s, 3H), 3.83-3.64 (m, 1H), 3.30-3.22 (m, | |
| | 1H), 3.04 (dd, J = 13.1, 10.5 Hz, 1H), 2.66 (s, 3H), 2.52 (s, 3H). | |
| 10 | 1H NMR (400 MHz, DMSO-d6) δ ppm 8.44 (d, J = 5.1 Hz, 1H), 7.66 (q, J = | 448.2 |
| | 7.8 Hz, 1H), 7.44-7.32 (m, 2H), 7.30-7.19 (m, 3H), 4.78-4.57 (m, 1H), | |
| | 4.58-4.39 (m, 1H), 4.32 (d, J = 12.9 Hz, 1H), 4.15 (d, J = 11.8 Hz, 1H), 3.81 | |
| | (t, J = 11.5 Hz, 1H), 3.07 (dd, J = 13.4, 10.1 Hz, 1H), 2.94-2.73 (m, 1H), | |
| | 2.64 (s, 3H), 2.52 (s, 3H), 2.49 (s, 3H). | |
| 11 | ¹H NMR (400 MHz, Chloroform-d) δ ppm 8.46 (3H, t, J = 5.2 Hz), 7.82 (1H, | 448.2 |
| | s), 7.72-7.68 (2H, m), 7.66-7.60 (3H, m), 7.22 (3H, s), 7.12 (3H, d, J = 5.1 | |
| | Hz), 7.08-7.00 (3H, m), 6.99-6.93 (3H, m), 4.89 (1H, dd, J = 8.6, 3.1 Hz), | |
| | 4.54 (2H, d, J = 11.2 Hz), 4.39-4.35 (2H, m), 3.96 (2H, dd, J = 6.6, 4.2 Hz), | |
| | 3.83 (2H, td, J = 11.4, 3.2 Hz), 3.53 (1H, t, J = 5.2 Hz), 3.41 (2H, tt, J = 11.7, | |
| | 4.0 Hz), 2.78 (3H, s), 2.75 (6H, s), 2.71 (3H, s), 2.67 (6H, s), 2.56 (10H, m), | |
| | 2.39 (2H, d, J = 13.4 Hz), 2.29-2.23 (3H, m), 2.14-2.05 (4H, m), 1.86-1.77 | |
| | (2H, m). | |
| 12 | ¹H NMR (400 MHz, Chloroform-d) δ ppm 8.46 (1H, d, J = 5.1 Hz), 7.69 (1H, | 448.2 |
| | s), 7.63 (1H, m), 7.26 (1H, s), 7.18 (1H, s), 7.05 (1H, m), 6.96 (1H, m), 4.56 | |
| | (1H, d, J = 11.2 Hz), 4.38 (1H, d, J = 11.5 Hz), 3.84 (1H, m), 3.41 (1H, m), | |
| | 2.75 (3H, s), 2.67 (3H, s), 2.60 (3H, s), 2.40 (1H, d, J = 13.3 Hz), 2.12 (2H, | |
| | m), 1.80 (1H, m). | |
| 13 | ¹H NMR (400 MHz, DMSO-d6) δ ppm 11.51-11.55 (1H, m), 7.35-7.38 (1H, | 487.2 |
| | m), 6.34 (1H, s), 6.19-6.22 (1H, m), 4.59-4.81 (1H, m), 4.38-4.41 (1H, m), | |
| | 4.07-4.11 (1H, m), 3.62-3.69 (1H, m), 3.16-3.25 (1H, m), 2.92-3.01 (1H, m), | |
| | 2.60-2.61 (3H, m), 2.59 (3H, s), 2.56 (6H, s). | |
| 14 | ¹H NMR (400 MHz, Chloroform-d): δ ppm 8.47 (1H, m), 7.53 (1H, s), 7.23 | 469.2 |
| | (1H, s), 7.13 (1H, m), 4.54 (1H, d, J = 11.3 Hz), 4.36 (1H, d, J = 11.4 Hz), | |
| | 3.86-3.80 (1H, m), 3.29 (1H, m), 2.73 (3H, s), 2.72 (3H, s) 2.60 (6H, s), 2.57 | |
| | (3H, s), 2.30 (1H, d, J = 13.5 Hz), 2.22-2.15 (1H, m), 2.05 (2H, m), 1.82-1.73 | |
| | (1H, m). | |
| 15 | ¹H NMR (400 MHz, DMSO-d6) δ ppm 7.79 (d, J = 7.9 Hz, 1H), 7.75 (d, J = | 479.0 |
| | 7.2 Hz, 1H), 7.69 (dd, J = 9.8, 2.0 Hz, 1H), 7.54 (dd, J = 8.3, 2.0 Hz, 1H), | |
| | 7.39 (s, 1H), 4.52 (dd, J = 11.4, 2.1 Hz, 1H), 4.11 (dd, J = 11.1, 4.2 Hz, 1H), | |
| | 3.71-3.77 (m, 1H), 3.62-3.71 (m, 1H), 3.43-3.53 (m, 1H), 2.79 (s, 3H), | |
| | 2.67 (s, 3H), 2.30 (s, 2H), 1.24 (s, 2H), 0.97-1.04 (m, 2H), 0.92 (td, J = 7.3, | |
| | 5.1 Hz, 2H) | |
| 16 | ¹H NMR (400 MHz, DMSO-d6) δ ppm 7.79 (d, J = 7.9 Hz, 1H), 7.75 (d, J = | 478.9 |
| | 7.2 Hz, 1H), 7.69 (dd, J = 9.8, 2.0 Hz, 1H), 7.54 (dd, J = 8.3, 2.0 Hz, 1H), | |
| | 7.39 (s, 1H), 4.52 (dd, J = 11.4, 2.1 Hz, 1H), 4.11 (dd, J = 11.1, 4.2 Hz, 1H), | |
| | 3.71-3.77 (m, 1H), 3.62-3.71 (m, 1H), 3.43-3.53 (m, 1H), 2.79 (s, 3H), | |
| | 2.67 (s, 3H), 2.30 (s, 2H), 1.24 (s, 2H), 0.97-1.04 (m, 2H), 0.92 (td, J = 7.3, | |
| | 5.1 Hz, 2H) | |
| 17 | ¹H NMR (DMSO-d6, 400 MHz): δH 9.57 (1H, s), 8.87-8.90 (1H, m), 8.18 | 498.2 |
| | (1H, d, J = 5.1 Hz), 8.13 (1H, d, J = 8.3 Hz), 7.07-7.08 (1H, m), 6.89 (1H, s), | |
| | 4.82 (2H, bd, J = 64.4 Hz), 4.64 (1H, d, J = 10.1 Hz), 4.13-4.18 (1H, m), 3.85 | |
| | (3H, s), 3.71-3.79 (1H, m), 3.00-3.12 (1H, m), 2.71-2.77 (1H, m), 2.65-2.67 | |
| | (3H, s), 2.61-2.61 (3H, s). | |
| 18 | ¹H NMR (400 MHz, Chloroform-d) δ ppm 8.46 (1H, d, J = 5.3 Hz), 8.07 (1H, | 434.2 |
| | dd, J = 8.5, 3.8 Hz), 7.69-7.63 (1H, m), 7.46-7.43 (1H, m), 7.32-7.31 (2H, | |
| | m), 7.17-7.10 (1H, m), 7.12-7.03 (1H, m), 4.85 (1H, dd, J = 9.7, 2.7 Hz), | |
| | 4.04-3.97 (2H, m), 3.97-3.93 (1H, m), 3.74-3.69 (1H, m), 2.88 (3H, s), | |
| | 2.62-2.57 (4H, m), 2.32-2.23 (1H, m), 2.22-2.14 (1H, m). | |
| 19 | ¹H NMR (400 MHz, Chloroform-d) δ ppm 8.48 (1H, d, J = 5.3 Hz), 8.22 (1H, | 467.2 |
| | m), 8.07 (1H, d, J = 8.2 Hz), 7.59 (1H, t, J = 7.9 Hz), 7.47-7.35 (4H, m), 4.86 | |
| | (1H, d, J = 9.9 Hz), 4.01-3.94 (2H, m), 3.72 (1H, m), 2.92-2.88 (4H, m), 2.63 | |
| | (4H, s), 2.25 (1H, m), 2.16 (1H, m). 19F NMR (376 MHz, Chloroform-d) | |
| | δF-110.9. | |
| 20 | ¹H NMR (400 MHz, Chloroform-d) δ ppm 8.46 (2H, m), 7.99 (1H, dd, J = 8.6, | 450.1 |
| | 3.3 Hz), 7.92 (1H, dd, J = 8.7, 3.5 Hz), 7.87 (1H, s), 7.73 (1H, s), 7.61-7.52 | |
| | (2H, m), 7.37-7.34 (1H, m), 7.32-7.29 (3H, m), 7.23-7.17 (2H, m), 7.12-7.05 | |
| | (2H, m), 7.03-6.97 (2H, m), 4.90-4.87 (1H, m), 4.56 (1H, dd, J = 11.2, 2.0 | |
| | Hz), 4.40-4.36 (1H, m), 3.98-3.94 (2H, m), 3.86-3.79 (1H, m), 3.54-3.51 (1H, | |
| | m), 3.42-3.35 (1H, m), 2.80 (3H, s), 2.77 (3H, s), 2.66-2.59 (7H, m), | |
| | 2.45-2.37 (2H, m), 2.32-2.20 (2H, m), 2.15-2.03 (2H, m), 1.87-1.75 (1H, m). | |
| 21 | ¹H NMR (400 MHz, Chloroform-d) δ ppm 8.46-8.43 (2H, m), 7.84 (1H, s), | 432.2 |
| | 7.74-7.71 (3H, m), 7.67-7.65 (3H, m), 7.54-7.47 (2H, m), 7.45-7.33 (2H, m), | |
| | 7.19-7.12 (3H, m), 4.88-4.85 (1H, m), 4.57-4.53 (1H, m), 4.39-4.34 (1H, m), | |
| | 3.98-3.94 (1H, m), 3.85-3.78 (2H, m), 3.52-3.49 (1H, m), 3.41-3.36 (1H, m), | |
| | 2.74 (6H, m), 2.58 (6H, s), 2.42 (6H, m), 2.33-2.16 (3H, m), 2.14-1.99 (4H, | |
| | m), 1.85-1.74 (1H, m). | |
| 22 | ¹H NMR (400 MHz, Chloroform-d) δ ppm 8.46 (2H, m), 7.84 (1H, s), | 462.2 |
| | 7.74-7.73 (1H, m), 7.71 (1H, s), 7.67 (1H, m), 7.58-7.49 (2H, m), 7.22-7.16 | |
| | (2H, m), 7.12-6.97 (6H, m), 4.90-4.86 (1H, m), 4.56-4.53 (1H, m), 4.39-4.34 | |
| | (1H, m), 3.97-3.94 (2H, m), 3.85-3.78 (1H, m), 3.54-3.50 (1H, m), 3.42-3.34 | |
| | (1H, m), 2.74 (3H, s), 2.71 (3H, s), 2.64-2.55 (6H, m), 2.45 (3H, s), 2.42 (3H, | |
| | s), 2.39-2.35 (2H, m), 2.32-2.18 (2H, m), 2.14-2.03 (3H, m), 1.84-1.73 (1H, m). | |
| 23 | ¹H NMR (400 MHz, Chloroform-d) δ ppm 8.48-8.46 (1H, m), 7.82 (1H, s), | 446.2 |
| | 7.71-7.67 (1H, m), 7.33 (2H, m), 7.08-7.03 (1H, m), 7.02-6.96 (1H, m), 4.96- | |
| | 4.92 (1H, m), 3.98 (2H, m), 3.56-3.53 (1H, m), 2.78 (3H, s), 2.70 (3H, s), | |
| | 2.66-2.60 (4H, m), 2.30-2.17 (3H, m). | |
| 24 | ¹H NMR (400 MHz, DMSO-d6) δ ppm 8.98 (d, J = 14.8 Hz, 1H), 7.83 (td, J = | 449.1 |
| | 8.4, 6.7 Hz, 1H), 7.74 (s, 1H), 7.53-7.43 (m, 1H), 7.39 (s, 1H), 7.33 (td, J = | |
| | 8.3, 2.3 Hz, 1H), 4.57-4.47 (m, 1H), 4.11 (dd, J = 11.4, 3.2 Hz, 1H), 3.78-3.60 | |
| | (m, 2H), 3.51 (s, 1H), 2.81 (s, 3H), 2.32 (d, J = 12.1 Hz, 1H), 2.08 (d, J = 12.8 | |
| | Hz, 1H), 2.02-1.85 (m, 2H), 1.03-0.84 (m, 4H). | |
| 25 | ¹H NMR (400 MHz, DMSO-d6) δ ppm 9.00 (s, 1H), 7.83 (td, J = 8.4, 6.7 Hz, | 449.1 |
| | 1H), 7.74 (s, 1H), 7.49 (td, J = 10.1, 2.5 Hz, 1H), 7.39 (s, 1H), 7.33 (td, J = | |
| | 8.4, 2.0 Hz, 1H), 4.58-4.46 (m, 1H), 4.11 (dd, J = 11.4, 3.2 Hz, 1H), 3.80-3.59 | |
| | (m, 2H), 3.51 (ddd, J = 12.1, 8.3, 3.6 Hz, 1H), 2.81 (s, 3H), 2.32 (d, J = 11.9 | |
| | Hz, 1H), 2.08 (d, J = 11.7 Hz, 1H), 1.99-1.88 (m, 2H), 1.04-0.97 (m, 2H), | |
| | 0.94-0.87 (m, 2H). | |
| 26 | ¹H NMR (400 MHz, Chloroform-d) δ ppm 8.46 (1H, d, J = 5.1 Hz), 7.69 (1H, | 448.2 |
| | s), 7.63 (1H, m), 7.26 (1H, s), 7.18 (1H, s), 7.05 (1H, m), 6.96 (1H, m), 4.56 | |
| | (1H, d, J = 11.2 Hz), 4.38 (1H, d, J = 11.5 Hz), 3.84 (1H, m), 3.41 (1H, m), | |
| | 2.75 (3H, s), 2.67 (3H, s), 2.60 (3H, s), 2.40 (1H, d, J = 13.3 Hz), 2.12 (2H, | |
| | m), 1.80 (1H, m). | |
| 27 | ¹H NMR (400 MHz, Chloroform-d) δ ppm 8.53 (s, 1H), 8.44 (d, J = 5.1 Hz, | 448.2 |
| | 1H), 7.72-7.64 (m, 1H), 7.41-7.33 (m, 2H), 7.29 (d, J = 6.0 Hz, 1H), | |
| | 7.26-7.20 (m, 2H), 4.66 (d, J = 8.9 Hz, 1H), 4.51 (d, J = 12.1 Hz, 1H), 4.36 | |
| | (d, J = 12.4 Hz, 1H), 4.16 (d, J = 10.9 Hz, 1H), 3.81 (t, J = 11.5 Hz, 1H), 3.11 | |
| | (t, J = 11.1 Hz, 1H), 2.92-2.82 (m, 1H), 2.65 (s, 3H). Note: One methyl | |
| | signal is obscured by solvent peak. 19F NMR (376 MHz, DMSO-d6) | |
| | δ ppm-108.64 (s), -109.12 (s). | |
| 28 | ¹H NMR (400 MHz, DMSO-d6) δ ppm 8.53 (s, 1H), 8.44 (d, J = 5.1 Hz, 1H), | 434.2 |
| | 7.72-7.63 (m, 1H), 7.42-7.34 (m, 2H), 7.29 (d, J = 4.2 Hz, 1H), 7.27-7.20 | |
| | (m, 2H), 4.66 (d, J = 10.2 Hz, 1H), 4.51 (d, J = 13.0 Hz, 1H), 4.36 (d, J = 12.1 | |
| | Hz, 1H), 4.16 (d, J = 8.5 Hz, 1H), 3.81 (t, J = 10.5 Hz, 1H), 3.11 (t, J = 10.6 | |
| | Hz, 1H), 2.93-2.82 (m, 1H), 2.65 (s, 3H). Note: One methyl signal is | |
| | obscured by solvent peak. 19F NMR (376 MHz, DMSO-d6) δ ppm-108.64 | |
| | (s), -109.12 (s). | |
| 29 | ¹H NMR (400 MHz, DMSO-d6) δ ppm 8.18 (d, J = 5.2 Hz, 1H), 7.74 (t, J = | 434.2 |
| | 7.9 Hz, 1H), 7.65 (dd, J = 9.8, 2.1 Hz, 1H), 7.50 (dd, J = 8.2, 2.0 Hz, 1H), | |
| | 7.05 (d, J = 5.3 Hz, 1H), 6.87 (s, 1H), 4.81 (d, J = 13.2 Hz, 1H), 4.65 (t, J = | |
| | 12.5 Hz, 2H), 4.14 (d, J = 11.5 Hz, 1H), 3.86 (s, 3H), 3.75 (t, J = 11.5 Hz, | |
| | 1H), 3.30-3.19 (m, 1H), 3.04 (dd, J = 13.2, 10.5 Hz, 1H), 2.65 (s, 3H), 2.52 | |
| | (s, 3H). | |
| 30 | ¹H NMR (400 MHz, DMSO-d6) δ ppm 8.18 (d, J = 5.2 Hz, 1H), 7.74 (t, J = | 481.1 |
| | 7.9 Hz, 1H), 7.65 (dd, J = 9.8, 2.0 Hz, 1H), 7.54-7.43 (m, 1H), 7.06 (d, J = | |
| | 5.3 Hz, 1H), 6.87 (s, 1H), 4.81 (d, J = 13.2 Hz, 1H), 4.65 (t, J = 12.5 Hz, 2H), | |
| | 4.20-4.08 (m, 1H), 3.86 (s, 3H), 3.79-3.64 (m, 1H), 3.26 (d, J = 14.6 Hz, | |
| | 1H), 3.04 (dd, J = 13.2, 10.5 Hz, 1H), 2.66 (s, 3H), 2.52 (s, 3H). | |
| 31 | v NMR (400 MHz, Chloroform-d) δ ppm 7.71 (1H, s), 7.61 (1H, t, J = 7.8 | 481.1 |
| | Hz), 7.48-7.45 (2H, m), 7.31 (1H, d, J = 8.4 Hz), 7.23 (1H, m), 4.56 (1H, d, | |
| | J = 11.2 Hz), 4.26 (1H, d, J = 11.5 Hz), 3.83-3.78 (1H, m), 3.57-3.53 (1H, m), | |
| | 3.34 (1H, m), 2.76 (3H, s), 2.68-2.67 (3H, s), 2.38 (1H, d, J = 13.2 Hz), | |
| | 2.08-1.93 (3H, m), 1.09 (2H, t, J = 3.9 Hz), 1.00-0.95 (2H, m). 19 F NMR | |
| | (376 MHz, Chloroform-d) δF-109.5 | |
| 32 | ¹H NMR (400 MHz, Chloroform-d) δ ppm 7.71 (1H, s), 7.61 (1H, t, J = 7.8 | 483.2 |
| | Hz), 7.48-7.45 (2H, m), 7.31 (1H, d, J = 8.4 Hz), 7.23 (1H, m), 4.56 (1H, d, | |
| | J = 11.2 Hz), 4.26 (1H, d, J = 11.5 Hz), 3.83-3.78 (1H, m), 3.57-3.53 (1H, m), | |
| | 3.34 (1H, m), 2.76 (3H, s), 2.68-2.67 (3H, s), 2.38 (1H, d, J = 13.2 Hz), 2.08- | |
| | 1.93 (3H, m), 1.09 (2H, t, J = 3.9 Hz), 1.00-0.95 (2H, m). 19 F NMR (376 | |
| | MHz, Chloroform-d) δF-109.5 | |
| 33 | ¹H NMR (400 MHz, DMSO-d6) δ ppm 8.98 (d, J = 14.8 Hz, 1H), 7.83 (td, | 449.1 |
| | J = 8.4, 6.7 Hz, 1H), 7.74 (s, 1H), 7.53-7.43 (m, 1H), 7.39 (s, 1H), 7.33 (td, J = | |
| | 8.3, 2.3 Hz, 1H), 4.57-4.47 (m, 1H), 4.11 (dd, J = 11.4, 3.2 Hz, 1H), 3.78-3.60 | |
| | (m, 2H), 3.51 (s, 1H), 2.81 (s, 3H), 2.32 (d, J = 12.1 Hz, 1H), 2.08 (d, J = 12.8 | |
| | Hz, 1H), 2.02-1.85 (m, 2H), 1.03-0.84 (m, 4H). | |
| 34 | ¹H NMR (400 MHz, DMSO-d6) δ ppm 9.00 (s, 1H), 7.83 (td, J = 8.4, 6.7 Hz, | 449.1 |
| | 1H), 7.74 (s, 1H), 7.49 (td, J = 10.1, 2.5 Hz, 1H), 7.39 (s, 1H), 7.33 (td, J = | |
| | 8.4, 2.0 Hz, 1H), 4.58-4.46 (m, 1H), 4.11 (dd, J = 11.4, 3.2 Hz, 1H), 3.80-3.59 | |
| | (m, 2H), 3.51 (ddd, J = 12.1, 8.3, 3.6 Hz, 1H), 2.81 (s, 3H), 2.32 (d, J = 11.9 | |
| | Hz, 1H), 2.08 (d, J = 11.7 Hz, 1H), 1.99-1.88 (m, 2H), 1.04-0.97 (m, 2H), | |
| | 0.94-0.87 (m, 2H). | |
| 35 | ¹H NMR (400 MHz, DMSO-d6) δ ppm 8.51 (s, 1H), 7.85 (s, 1H), 7.66 (td, J = | 483.2 |
| | 8.4, 6.6 Hz, 1H), 7.48 (s, 1H), 7.36 (td, J = 9.8, 2.5 Hz, 1H), 7.23 (td, J = 8.6, | |
| | 2.6 Hz, 1H), 7.18 (s, 1H), 4.56 (dd, J = 10.4, 2.7 Hz, 1H), 4.41 (d, J = 13.2 | |
| | Hz, 1H), 4.29-4.19 (m, 1H), 4.09-3.86 (m, 1H), 3.89-3.52 (m, 2H), | |
| | 3.21-2.84 (m, 2H), 2.64 (s, 3H), 1.11-0.98 (m, 2H), 0.98-0.89 (m, 2H). | |
| 36 | ¹H NMR (400 MHz, DMSO-d6) δ ppm 7.84 (s, 1H), 7.65 (td, J = 8.4, 6.7 Hz, | 449.2 |
| | 1H), 7.47 (s, 1H), 7.36 (td, J = 9.8, 2.5 Hz, 1H), 7.22 (td, J = 9.5, 9.0, 3.1 Hz, | |
| | 1H), 7.18 (s, 1H), 4.55 (dd, J = 10.5, 2.7 Hz, 1H), 4.37 (d, J = 12.6 Hz, 1H), | |
| | 4.22 (d, J = 12.8 Hz, 1H), 4.02 (dd, J = 11.6, 3.2 Hz, 1H), 3.82-3.55 (m, | |
| | 2H), 3.18-2.84 (m, 2H), 2.64 (s, 3H), 2.51 (s, 3H), 1.08-0.99 (m, 2H), | |
| | 0.98-0.89 (m, 2H). | |
| 37 | ¹H NMR (400 MHz, CD2Cl2) δ ppm 8.45 (d, J = 4.7 Hz, 1H), 7.86 (s, 1H), | 463.2 |
| | 7.67 (d, J = 8.2 Hz, 1H), 7.52 (d, J = 8.2 Hz, 1H), 7.25 (s, 1H), 7.17 (s, 1H), | |
| | 5.01 (d, J = 11.9 Hz, 1H), 4.83 (d, J = 13.0 Hz, 1H), 4.54 (d, J = 10.8 Hz, 1H), | |
| | 4.17 (d, J = 9.9 Hz, 1H), 3.81 (t, J = 10.5 Hz, 1H), 3.28 (t, J = 12.4 Hz, 1H), | |
| | 3.00 (dd, J = 13.3, 10.7 Hz, 1H), 2.67 (s, 3H), 2.54 (s, 3H), 2.51 (s, 3H). 19F | |
| | NMR (376 MHz, CD2Cl2) δ ppm-57.89 (s). | |
| 38 | ¹H NMR (400 MHz, CD2Cl2) δ ppm 8.46 (d, J = 5.2 Hz, 1H), 7.64 (s, 1H), | 515.20 |
| | 7.50 (t, J = 7.9 Hz, 1H), 7.40-7.30 (m, 3H), 7.26 (s, 2H), 4.66 (dd, J = 10.3, | |
| | 1.6 Hz, 1H), 4.53 (dd, J = 12.1, 1.7 Hz, 1H), 4.27-4.17 (m, 2H), 3.90 (td, J = | |
| | 11.7, 2.9 Hz, 1H), 3.19 (t, J = 11.1 Hz, 1H), 2.93-2.84 (m, 1H), 2.70 (s, 3H), | |
| | 2.60 (s, 3H), 2.34 (s, 3H). 19F NMR (376 MHz, CD2Cl2) δ ppm-112.30 (s). | |
| 39 | ¹H NMR (400 MHz, CD2Cl2) δ ppm 8.46 (d, J = 5.2 Hz, 1H), 7.64 (s, 1H), | 463.2 |
| | 7.50 (t, J = 7.9 Hz, 1H), 7.40-7.30 (m, 3H), 7.26 (s, 2H), 4.66 (dd, J = 10.3, | |
| | 1.6 Hz, 1H), 4.53 (dd, J = 12.1, 1.7 Hz, 1H), 4.27-4.17 (m, 2H), 3.90 (td, | |
| | J = 11.7, 2.9 Hz, 1H), 3.19 (t, J = 11.1 Hz, 1H), 2.93-2.84 (m, 1H), 2.70 (s, | |
| | 3H), 2.60 (s, 3H), 2.34 (s, 3H). ¹⁹F NMR (376 MHz, CD2Cl2) δ ppm-112.30. | |
| 40 | ¹H NMR (400 MHz, Chloroform-d): δ ppm 9.84 (1H, s), 8.95 (1H, d, J = 8.3 | 481.2 |
| | Hz), 8.47-8.44 (1H, m), 7.91-7.87 (1H, m), 7.26-7.24 (3H, m), 7.15-7.08 (1H, | |
| | m), 4.58-4.52 (1H, m), 4.41-4.37 (1H, m), 3.89-3.83 (1H, m), 3.67-3.59 (1H, | |
| | m), 2.87 (3H, s), 2.81 (3H, s), 2.56 (3H, s), 2.50-2.46 (1H, m), 2.29-2.21 (2H, | |
| | m), 2.09-2.00 (1H, m). 19F NMR (376 MHz, Chloroform-d): δ ppm-68.1. | |
| 41 | ¹H NMR (400 MHz, CD2Cl2) δ ppm 9.03 (s, 1H), 8.46 (d, J = 5.1 Hz, 1H), | 447.2 |
| | 8.20 (d, J = 8.0 Hz, 1H), 7.87 (d, J = 8.0 Hz, 1H), 7.80 (s, 1H), 7.27 (s, 1H), | |
| | 7.19 (d, J = 4.9 Hz, 1H), 7.10 (s, 1H), 4.65 (dd, J = 10.4, 2.5 Hz, 1H), 4.50 | |
| | (d, J = 13.0 Hz, 1H), 4.29-4.17 (m, 2H), 3.96-3.87 (m, 1H), 3.22-3.11 | |
| | (m, 1H), 2.87 (dd, J = 12.7, 10.6 Hz, 1H), 2.64 (s, 3H), 2.56 (s, 3H), 2.35 (s, | |
| | 3H). 19F NMR (376 MHz, CD2Cl2) δ ppm-68.18 (s) | |
| 42 | ¹H NMR (400 MHz, CD2Cl2) δ ppm 9.03 (s, 1H), 8.46 (d, J = 5.1 Hz, 1H), | 480.2 |
| | 8.20 (d, J = 8.0 Hz, 1H), 7.87 (d, J = 8.0 Hz, 1H), 7.80 (s, 1H), 7.27 (s, 1H), | |
| | 7.19 (d, J = 4.9 Hz, 1H), 7.10 (s, 1H), 4.65 (dd, J = 10.4, 2.5 Hz, 1H), 4.50 | |
| | (d, J = 13.0 Hz, 1H), 4.29-4.17 (m, 2H), 3.96-3.87 (m, 1H), 3.22-3.11 | |
| | (m, 1H), 2.87 (dd, J = 12.7, 10.6 Hz, 1H), 2.64 (s, 3H), 2.56 (s, 3H), 2.35 (s, | |
| | 3H). ¹⁹F NMR (376 MHz, CD2Cl2) δ ppm-68.18. | |
| 43 | ¹H NMR (400 MHz, DMSO-d6) δ ppm 9.48 (d, J = 2.0 Hz, 1H), 8.78 (dd, J = | 479.1 |
| | 8.1, 2.1 Hz, 1H), 8.07 (d, J = 8.2 Hz, 1H), 7.86 (s, 1H), 7.50 (s, 1H), 7.25 (s, | |
| | 1H), 4.58 (dd, J = 10.5, 2.7 Hz, 1H), 4.46 (d, J = 12.7 Hz, 1H), 4.32 (d, J = | |
| | 12.8 Hz, 1H), 4.14-3.98 (m, 1H), 3.75 (dd, J = 10.7, 2.3 Hz, 1H), 3.69 | |
| | (dq, J = 7.4, 3.8 Hz, 1H), 3.09 (dd, J = 12.5, 3.5 Hz, 1H), 3.02 (dd, J = 12.9, | |
| | 10.5 Hz, 1H), 2.66 (s, 3H), 2.61 (s, 3H), 1.06-1.00 (m, 2H), 0.95 (td, J = 7.4, | |
| | 5.1 Hz, 2H). | |
| 44 | ¹H NMR (400 MHz, CD2Cl2) δ ppm 9.67-9.68 (1H, m), 8.87-8.90 (1H, m), | 496.2 |
| | 7.86-7.88 (1H, m), 7.28-7.31 (1H, m), 6.61-6.63 (1H, m), 6.33-6.35 (1H, m), | |
| | 5.02-5.09 (1H, m), 4.86-4.91 (1H, m), 4.43-4.47 (1H, m), 4.17-4.21 (1H, m), | |
| | 3.78-3.85 (1H, m), 3.32-3.36 (1H, m), 3.04-3.11 (1H, m), 2.71 (3H, s), 2.65 | |
| | (3H, s). Note: The exchangeable proton was not observed. 19F NMR | |
| | (CH2Cl2-d2, 376 MHz) δ ppm-68.4 | |
| 45 | ¹H NMR (400 MHz, DMSO-d6) δ ppm 9.00 (s, 1H), 7.78-7.84 (m, 1H), | 465.0 |
| | 7.74 (s, 1H), 7.70 (d, J = 9.9 Hz, 1H), 7.55 (d, J = 8.3 Hz, 1H), 7.40 (s, 1H), | |
| | 4.53 (d, J = 9.9 Hz, 1H), 4.16-4.08 (m, 1H), 3.74-3.62 (m, 2H), 3.56-3.45 | |
| | (m, 1H), 2.82 (s, 3H), 2.31 (d, J = 13.2 Hz, 1H), 2.08 (d, J = 13.1 Hz, | |
| | 1H), 1.87-1.99 (m, 2H), 1.06-0.96 (m, 2H), 0.94-0.83 (m, 2H) | |
| 46 | ¹H NMR (400 MHz, DMSO-d6) δ ppm 9.00 (s, 1H), 7.78-7.84 (m, 1H), | 465.0 |
| | 7.74 (s, 1H), 7.70 (d, J = 9.9 Hz, 1H), 7.55 (d, J = 8.3 Hz, 1H), 7.40 (s, 1H), | |
| | 4.53 (d, J = 9.9 Hz, 1H), 4.16-4.08 (m, 1H), 3.74-3.62 (m, 2H), 3.56-3.45 | |
| | (m, 1H), 2.82 (s, 3H), 2.31 (d, J = 13.2 Hz, 1H), 2.08 (d, J = 13.1 Hz, 1H), | |
| | 1.87-1.99 (m, 2H), 1.06-0.96 (m, 2H), 0.94-0.83 (m, 2H) | |
| 47 | ¹H NMR (400 MHz, CD2Cl2) δ ppm 8.46 (d, J = 5.0 Hz, 1H), 7.49-7.42 (m, | 483.2 |
| | 1H), 7.39-7.34 (m, 1H), 7.27 (s, 1H), 7.19 (d, J = 4.4 Hz, 1H), 5.02 (d, J = | |
| | 13.6 Hz, 1H), 4.85 (d, J = 13.3 Hz, 1H), 4.56 (dd, J = 10.6, 1.5 Hz, 1H), 4.18 | |
| | (dd, J = 12.0, 2.5 Hz, 1H), 3.88-3.77 (m, 1H), 3.36-3.25 (m, 1H), 3.03 | |
| | (dd, J = 13.3, 10.7 Hz, 1H), 2.69 (s, 3H), 2.57 (s, 3H), 2.55 (s, 3H). 19F NMR | |
| | (376 MHz, CD2Cl2) δ ppm-133.16 (s), -139.03 (s). | |
| 48 | ¹H NMR (600 MHz, DMSO-d6) δ ppm 8.56 (s, 1H), 8.43-8.51 (m, 1H), | 457.0 |
| | 7.35 (s, 1H), 7.25-7.32 (m, 1H), 4.87 (br d, J = 11.63 Hz, 1H), 4.61 (br dd, = | |
| | 10.44, 2.45 Hz, 1H), 4.15 (br dd, J = 11.90, 2.27 Hz, 1H), 3.68-3.77 (m, | |
| | 1H), 2.98-3.10 (m, 1H), 2.64 (s, 3H), 2.58 (s, 5H), 2.52-2.53 (m, 1H), | |
| | 2.43-2.49 (m, 1H), 0.97-1.07 (m, 1H) | |
| 49 | ¹H NMR (400 MHz, CDCl3) δ ppm 8.38 (s, 1H), 7.66-7.36 (m, 2H), 5.06 (s, | 486.0 |
| | 1H), 4.70 (dt, J = 100.5, 50.4 Hz, 2H), 4.05-3.68 (m, 2H), 3.49 (dq, J = 7.3, | |
| | 3.8 Hz, 1H), 3.27 (d, J = 87.1 Hz, 1H), 2.70 (s, 3H), 2.59 (d, J = 10.7 Hz, 6H), | |
| | 1.24 (s, 3H), 1.02 (dd, J = 8.4, 4.9 Hz, 2H), 0.93 (t, J = 16.8 Hz, 2H). | |
| 50 | ¹H NMR (400 MHz, CDCl3) δ ppm 8.38 (s, 1H), 7.54 (d, J = 8.6 Hz, 2H), | 486.0 |
| | 5.06 (d, J = 44.1 Hz, 2H), 4.57 (dd, J = 10.9, 2.4 Hz, 1H), 3.80 (ddd, J = 10.6, | |
| | 6.3, 2.5 Hz, 1H), 3.65-3.52 (m, 1H), 3.05 (s, 1H), 2.81 (dd, J = 13.2, 10.8 Hz, | |
| | 1H), 2.71 (s, 3H), 2.58 (s, 6H), 1.33 (d, J = 4.2 Hz, 3H), 1.13 (s, 2H), 0.99 | |
| | (dd, J = 27.6, 6.8 Hz, 2H). | |
| 51 | ¹H NMR (400 MHz, CDCl3) δ ppm 7.46 (s, 2H), 5.05 (t, J = 3.5 Hz, 1H), 4.86 | 500.0 |
| | (s, 1H), 4.60 (s, 1H), 3.90 (s, 2H), 3.49 (ddd, J = 11.1, 7.3, 3.8 Hz, 1H), 3.15 | |
| | (s, 1H), 2.70 (d, J = 14.3 Hz, 3H), 2.63 (s, 3H), 2.60 (s, 6H), 1.26-1.22 (m, | |
| | 3H), 1.05-0.99 (m, 2H), 0.95 (d, J = 14.3 Hz, 2H). | |
| 52 | ¹H NMR (400 MHz, CDCl3) δ ppm 7.65 (dd, J = 14.9, 8.2 Hz, 1H), 7.53 (d, | 477.0 |
| | J = 6.8 Hz, 2H), 7.05-6.88 (m, 3H), 4.69 (dd, J = 10.8, 2.6 Hz, 1H), 4.44 (d, J = | |
| | 12.7 Hz, 1H), 4.27 (d, J = 12.7 Hz, 1H), 3.95-3.86 (m, 1H), 3.58 (ddd, J = | |
| | 11.0, 7.3, 3.8 Hz, 1H), 2.99-2.92 (m, 1H), 2.79-2.65 (m, 4H), 2.60 (s, 3H), | |
| | 1.34 (d, J = 6.2 Hz, 3H), 1.12 (dd, J = 7.0, 4.4 Hz, 2H), 1.00 (t, J = 5.9 Hz, | |
| | 2H). | |
| 53 | ¹H NMR (400 MHz, CDCl3) δ ppm 7.65 (dd, J = 14.9, 8.2 Hz, 1H), 7.54 (s, | 477.0 |
| | 2H), 7.04-6.91 (m, 3H), 4.69 (dd, J = 10.8, 2.5 Hz, 1H), 4.45 (d, J = 12.8 Hz, | |
| | 1H), 4.27 (d, J = 12.6 Hz, 1H), 3.96-3.88 (m, 1H), 3.60-3.55 (m, 1H), | |
| | 3.01-2.93 (m, 1H), 2.78-2.68 (m, 4H), 2.60 (s, 3H), 1.34 (d, J = 6.2 Hz, 3H), | |
| | 1.12 (dd, J = 7.1, 4.4 Hz, 2H), 1.00 (t, J = 6.1 Hz, 2H). | |
| 54 | ¹H NMR (600 MHz, DMSO-d6) δ ppm 8.53-8.56 (m, 1H), 7.84 (s, 1H), | 472.0 |
| | 7.47 (s, 1H), 4.76 (br d, J = 12.53 Hz, 1H), 4.62 (br s, 1H), 4.49 (dd, J = | |
| | 10.35, 2.72 Hz, 1H), 3.98-4.05 (m, 1H), 3.68-3.73 (m, 1H), 3.60-3.68 | |
| | (m, 1H), 3.14-3.25 (m, 1H), 2.63 (s, 3H), 2.57 (s, 6H), 1.19-1.32 (m, 1H), | |
| | 0.93-1.05 (m, 5H). | |
| 55 | ¹H NMR (400 MHz, CDCl3) δ ppm 8.75 (d, J = 7.1 Hz, 1H), 7.56 (d, J = 3.9 | 471.0 |
| | Hz, 1H), 7.47 (d, J = 15.4 Hz, 1H), 4.86-4.13 (m, 2H), 3.88-3.75 (m, 1H), | |
| | 3.51 (t, J = 33.6 Hz, 2H), 2.81 (d, J = 5.2 Hz, 3H), 2.59 (s, 7H), 2.32 (d, J = | |
| | 13.7 Hz, 1H), 2.08 (dd, J = 20.6, 8.9 Hz, 2H), 1.03 (d, J = 5.0 Hz, 4H). | |
| 56 | ¹H NMR (400 MHz, CDCl3) δ ppm 8.48 (s, 1H), 7.66-7.60 (m, 2H), 7.52 (s, | 463.0 |
| | 1H), 7.50 (d, J = 3.1 Hz, 2H), 7.00 (s, 1H), 5.08 (s, 1H), 4.51-4.47 (m, 1H), | |
| | 4.01-3.99 (m, 1H), 3.73 (d, J = 9.2 Hz, 1H), 3.53 (d, J = 3.6 Hz, 2H), | |
| | 3.25-3.17 (m, 2H), 2.87 (s, 3H), 1.28 (d, J = 6.3 Hz, 3H), 1.06 (d, J = 4.0 Hz, | |
| | 1H), 0.98 (d, J = 5.3 Hz, 2H). | |
| 57 | ¹H NMR (400 MHz, CDCl3) δ ppm 8.48 (s, 1H), 7.66-7.60 (m, 2H), 7.52 (s, | 462.9 |
| | 1H), 7.50 (d, J = 3.1 Hz, 2H), 7.00 (s, 1H), 5.08 (s, 1H), 4.51-4.47 (m, 1H), | |
| | 4.01-3.99 (m, 1H), 3.73 (d, J = 9.2 Hz, 1H), 3.53 (d, J = 3.6 Hz, 2H), | |
| | 3.25-3.17 (m, 2H), 2.87 (s, 3H), 1.28 (d, J = 6.3 Hz, 3H), 1.06 (d, J = 4.0 Hz, | |
| | 1H), 0.98 (d, J = 5.3 Hz, 2H). | |
| 58 | ¹H NMR (400 MHz, CD2Cl2) δ ppm 8.47 (d, J = 4.9 Hz, 1H), 7.49-7.41 (m, | 483.2 |
| | 1H), 7.39-7.33 (m, 1H), 7.27 (s, 1H), 7.19 (d, J = 3.7 Hz, 1H), 5.02 (d, J = | |
| | 13.0 Hz, 1H), 4.86 (d, J = 13.1 Hz, 1H), 4.56 (d, J = 10.2 Hz, 1H), 4.19 | |
| | (dd, J = 11.8, 2.1 Hz, 1H), 3.88-3.77 (m, 1H), 3.37-3.26 (m, 1H), 3.03 | |
| | (dd, J = 13.2, 10.7 Hz, 1H), 2.69 (s, 3H), 2.57 (s, 3H), 2.55 (s, 3H). 19F NMR | |
| | (376 MHz, CD2Cl2) δ ppm-133.15 (s), -139.04 (s). | |
| 59 | ¹H NMR (400 MHz, CD2Cl2) δ ppm 11.45 (s, 1H), 7.34 (d, J = 6.8 Hz, 1H), | 483.1 |
| | 6.61 (s, 1H), 6.35 (d, J = 8.2 Hz, 1H), 4.99 (d, J = 13.7 Hz, 1H), 4.82 (d, J = | |
| | 12.8 Hz, 1H), 4.41 (d, J = 7.8 Hz, 1H), 4.15 (d, J = 11.3 Hz, 1H), 3.81-3.72 | |
| | (m, 1H), 3.31-3.20 (m, 1H), 3.06-2.95 (m, 1H), 2.65 (s, 3H), 2.62 (s, 3H), | |
| | 2.60 (s, 6H). 19F NMR (376 MHz, CD2Cl2) δ ppm-73.46 (s) | |
| 60 | ¹H NMR (400 MHz, CDCl3) δ ppm 7.42 (t, J = 12.8 Hz, 2H), 4.77 (dd, J = | 485.2 |
| | 8.0, 3.4 Hz, 1H), 3.92-3.73 (m, 2H), 3.64-3.41 (m, 2H), 2.72 (t, J = 11.3 Hz, | |
| | 6H), 2.57 (s, 6H), 2.39-2.30 (m, 1H), 2.28-2.18 (m, 1H), 2.08 (qd, J = 8.7, 4.6 | |
| | Hz, 1H), 1.18 (s, 1H), 1.06-1.01 (m, 2H), 0.93 (qd, J = 5.5, 1.2 Hz, 2H). | |
| 61 | ¹H NMR (400 MHz, CDCl3) δ ppm 7.44 (t, J = 5.8 Hz, 2H), 4.47 (dd, J = | 485.3 |
| | 11.4, 1.9 Hz, 1H), 4.18 (dt, J = 6.0, 3.3 Hz, 1H), 3.79-3.66 (m, 1H), 3.49 | |
| | (tt, J = 7.3, 3.8 Hz, 1H), 3.37 (ddd, J = 15.8, 11.8, 3.7 Hz, 1H), 2.79-2.65 (m, | |
| | 6H), 2.58 (s, 6H), 2.30 (d, J = 13.2 Hz, 1H), 2.07 (ddd, J = 11.6, 9.9, 4.2 Hz, | |
| | 3H), 1.20(d, J = 12.5 Hz, 1H), 1.06-0.96 (m, 2H), 0.92(td, J = 7.1, 4.9 Hz, | |
| | 2H). | |
| 62 | ¹H NMR (400 MHz, CDCl3) δ ppm 7.70-7.56 (m, 5H), 7.55-7.47 (m, 8H), | 493.0 |
| | 7.00 (s, 2H), 6.95 (s, 3H), 5.07 (s, 3H), 4.33 (s, 3H), 3.91 (d, J = 6.4 Hz, 4H), | |
| | 3.66-3.58 (m, 3H), 3.51 (dd, J = 7.4, 3.6 Hz, 3H), 3.13-3.02 (m, 3H), 2.77 (s, | |
| | 4H), 2.77 (s, 4H), 2.69 (d, J = 1.9 Hz, 13H), 2.60 (s, 10H), 1.27 (d, J = 6.4 | |
| | Hz, 11H), 1.04 (d, J = 4.2 Hz, 7H), 1.00-0.89 (m, 7H). | |
| 63 | ¹H NMR (400 MHz, CDCl3) δ ppm 8.43 (d, J = 4.4 Hz, 1H), 7.58 (t, J = 7.9 | 478.9 |
| | Hz, 1H), 7.54 (s, 2H), 7.31-7.28 (m, 1H), 7.25-7.22 (m, 1H), 6.95 (s, 1H), | |
| | 4.68 (dd, J = 10.9, 2.5 Hz, 1H), 4.46 (d, J = 12.5 Hz, 1H), 4.29 (d, J = 12.4 | |
| | Hz, 1H), 3.94-3.86 (m, 1H), 3.57 (td, J = 7.3, 3.7 Hz, 1H), 2.99 (dd, J = 12.7, | |
| | 11.0 Hz, 1H), 2.77 (dd, J = 12.6, 10.7 Hz, 1H), 2.70 (s, 3H), 1.34 (d, J = 6.2 | |
| | Hz, 3H), 1.12 (td, J = 7.3, 4.4 Hz, 2H), 1.04-0.99 (m, 2H). | |
| 64 | ¹H NMR (400 MHz, CDCl3) δ 7.72 (dd, J = 14.8, 8.1 Hz, 1H), 7.51 (d, J = 9.1 | 478.1 |
| | Hz, 2H), 7.11-6.89 (m, 2H), 5.06 (s, 1H), 4.75 (d, J = 69.4 Hz, 2H), 3.86 (d, | |
| | J = 64.9 Hz, 2H), 3.49 (s, 1H), 3.21 (s, 1H), 2.72 (s, 3H), 2.60 (s, 3H), 1.25 (d, | |
| | J = 6.3 Hz, 3H), 1.01 (s, 2H), 0.93 (dd, J = 6.7, 3.1 Hz, 2H). | |
| 65 | ¹H NMR (400 MHz, CDCl3) δ 8.41 (s, 1H), 7.69 (dd, J = 14.8, 7.8 Hz, 1H), | 464.0 |
| | 7.50 (dd, J = 27.7, 12.3 Hz, 2H), 7.03 (dt, J = 17.4, 8.2 Hz, 2H), 5.07 (s, 1H), | |
| | 4.83 (t, J = 28.4 Hz, 1H), 4.70 (d, J = 12.6 Hz, 1H), 4.06-3.72 (m, 2H), | |
| | 3.58-3.17 (m, 2H), 2.74 (s, 3H), 1.26 (d, J = 6.3 Hz, 3H), 1.01 (s, 2H), | |
| | 0.94 (d, J = 6.6 Hz, 2H). | |
| 66 | ¹H NMR (400 MHz, CDCl3) δ 8.47-8.35 (m, 1H), 7.68 (dd, J = 14.5, 7.4 Hz, | 464.0 |
| | 1H), 7.53 (d, J = 5.3 Hz, 2H), 7.13-6.92 (m, 2H), 5.04 (s, 2H), 4.60 (d, J = | |
| | 10.6 Hz, 1H), 3.91-3.76 (m, 1H), 3.57 (ddd, J = 10.9, 7.3, 3.7 Hz, 1H), 3.10 | |
| | (dd, J = 13.4, 11.0 Hz, 1H), 2.86 (dd, J = 13.4, 10.7 Hz, 1H), 2.74 (s, 3H), | |
| | 1.33 (d, J = 6.2 Hz, 3H), 1.17-1.07 (m, 2H), 1.06-0.96 (m, 2H). | |
| 67 | ¹H NMR (400 MHz, CDCl3) δ ppm 7.65 (t, J = 7.8 Hz, 1H), 7.53 (t, J = 4.1 | 494.0 |
| | Hz, 2H), 7.30 (t, J = 6.0 Hz, 1H), 7.24 (d, J = 2.0 Hz, 1H), 4.97 (s, 2H), 4.60 | |
| | (d, J = 8.7 Hz, 1H), 3.89-3.76 (m, 1H), 3.57 (ddd, J = 11.1, 7.3, 3.9 Hz, 1H), | |
| | 3.07 (dd, J = 13.3, 11.0 Hz, 1H), 2.84 (dd, J = 13.4, 10.7 Hz, 1H), 2.71 (s, | |
| | 3H), 2.59 (s, 3H), 1.32 (d, J = 6.2 Hz, 3H), 1.15-1.07 (m, 2H), 1.01 (dt, J = | |
| | 12.3, 6.3 Hz, 2H). | |
| 68 | ¹H NMR (400 MHz, CDCl3) δ 7.65 (t, J = 7.8 Hz, 1H), 7.53 (t, J = 4.5 Hz, | 494.0 |
| | 2H), 7.33-7.28 (m, 1H), 7.24 (d, J = 1.9 Hz, 1H), 4.97 (s, 2H), 4.60 (d, J = 8.6 | |
| | Hz, 1H), 3.88-3.77 (m, 1H), 3.62-3.53 (m, 1H), 3.07 (dd, J = 13.3, 11.0 Hz, | |
| | 1H), 2.84 (dd, J = 13.3, 10.7 Hz, 1H), 2.71 (s, 3H), 2.59 (s, 3H), 1.32 (d, J = | |
| | 6.2 Hz, 3H), 1.16-1.08 (m, 2H), 1.01 (q, J = 6.7 Hz, 2H). | |
| 69 | ¹H NMR (400 MHz, CDCl3) δ ppm 8.41 (s, 1H), 7.62 (s, 1H), 7.58-7.50 (m, | 481.0 |
| | 2H), 7.38-7.27 (m, 2H), 5.04 (s, 2H), 4.60 (d, J = 9.1 Hz, 1H), 3.83 (s, 1H), | |
| | 3.58 (s, 1H), 3.09 (dd, J = 13.4, 11.0 Hz, 1H), 2.86 (dd, J = 13.3, 10.7 Hz, | |
| | 1H), 2.74 (s, 3H), 1.33 (d, J = 6.2 Hz, 3H), 1.18-1.07 (m, 2H), 1.06-0.95 (m, | |
| | 2H). | |
| 70 | ¹H NMR (400 MHz, CDCl3) δ ppm 8.41 (s, 1H), 7.62 (s, 1H), 7.53 (d, J = 5.8 | 481.0 |
| | Hz, 2H), 7.30 (dd, J = 15.3, 7.4 Hz, 2H), 5.04 (s, 2H), 4.60 (d, J = 9.4 Hz, | |
| | 1H), 3.83 (s, 1H), 3.58 (s, 1H), 3.10 (dd, J = 13.4, 11.0 Hz, 1H), 2.86 (dd, J = | |
| | 13.4, 10.7 Hz, 1H), 2.74 (s, 3H), 1.33 (d, J = 6.2 Hz, 3H), 1.12 (dt, J = 8.3, | |
| | 4.3 Hz, 2H), 1.06-0.98 (m, 2H). | |
| 71 | ¹H NMR (400 MHz, Chloroform-d) δ ppm 7.70 (1H, s), 7.68-7.62 (1H, m), | 456.20 |
| | 7.49 (2H, s), 7.05 (1H, t, J = 8.4 Hz), 6.96 (1H, td, J = 9.4, 2.4 Hz), 4.61 (1H, | |
| | d, J = 11.2 Hz), 3.85 (1H, dd, J = 10.8, 6.0 Hz), 3.54 (1H, tt, J = 7.2, 3.8 Hz), | |
| | 2.76 (3H, s), 3.39-3.33 (1H, m), 2.68 (3H, s), 2.35 (1H, d, J = 13.1 Hz), 2.15 | |
| | (1H, d, J = 13.1 Hz), 1.93 (1H, q, J = 12.2 Hz), 1.70-1.61 (1H, m), 1.33 (3H, | |
| | d, J = 6.2 Hz), 1.09 (2H, t, J = 3.5 Hz), 0.99 (2H, t, J = 6.6 Hz). 19F | |
| | NMR (376 MHz, Chloroform-d) δ ppm-109.3, -109.3, -109.2, -109.2, -109.2, | |
| | -109.2, -109.2, -107.7, -107.7, -107.7, -107.7. | |
| 72 | ¹H NMR (400 MHz, CDCl3) δ ppm 8.33 (d, J = 7.0 Hz, 1H), 7.56-7.42 (m, | 485.0 |
| | 2H), 6.69 (d, J = 21.2 Hz, 1H), 4.60 (dd, J = 10.8, 2.6 Hz, 1H), 4.37 (d, J = | |
| | 12.6 Hz, 1H), 4.19 (d, J = 12.3 Hz, 1H), 3.82 (ddd, J = 10.4, 6.4, 2.6 Hz, 1H), | |
| | 3.52 (tt, J = 7.3, 3.8 Hz, 1H), 2.95-2.75 (m, 1H), 2.70-2.57 (m, 4H), 2.52-2.47 | |
| | (m, 6H), 1.27 (d, J = 6.2 Hz, 3H), 1.07 (td, J = 7.3, 4.6 Hz, 2H), 0.98-0.92 (m, | |
| | 2H). | |
| 73 | ¹H NMR (400 MHz, CDCl3) δ 7.47 (s, 2H), 6.73 (s, 1H), 4.61 (dd, J = 10.8, | 499.1 |
| | 2.6 Hz, 1H), 4.34 (d, J = 12.6 Hz, 1H), 4.16 (d, J = 12.5 Hz, 1H), 3.88-3.78 | |
| | (m, 1H), 3.52 (ddd, J = 11.1, 7.4, 3.8 Hz, 1H), 2.88-2.79 (m, 1H), 2.65-2.60 | |
| | (m, 1H), 2.58 (s, 6H), 2.50 (s, 5H), 1.26 (d, J = 6.2 Hz, 3H), 1.18 (s, 1H), | |
| | 1.09-1.04 (m, 2H), 0.98-0.93 (m, 2H). | |
| 74 | ¹H NMR (400 MHz, CDCl3) δ ppm 7.47 (s, 2H), 6.73 (s, 1H), 4.61 (dd, J = | 499.1 |
| | 10.8, 2.6 Hz, 1H), 4.34 (d, J = 12.6 Hz, 1H), 4.16 (d, J = 12.5 Hz, 1H), | |
| | 3.88-3.78 (m, 1H), 3.52 (ddd, J = 11.1, 7.4, 3.8 Hz, 1H), 2.88-2.79 (m, 1H), | |
| | 2.65-2.60 (m, 1H), 2.58 (s, 6H), 2.50 (s, 5H), 1.26 (d, J = 6.2 Hz, 3H), | |
| | 1.18 (s, 1H), 1.09-1.04 (m, 2H), 0.98-0.93 (m, 2H). | |
| 75 | ¹H NMR (400 MHz, Chloroform-d) δ ppm 9.58 (s, 1H), 8.78 (dd, J = 8.0, 2.1 | 476.2 |
| | Hz, 1H), 7.81 (d, J = 8.2 Hz, 1H), 7.54 (s, 1H), 7.48 (s, 1H), 7.01 (s, 1H), 4.64 | |
| | (dd, J = 10.4, 2.8 Hz, 1H), 4.52-4.37 (m, 1H), 4.22 (d, J = 12.7 Hz, 1H), | |
| | 4.17-4.06 (m, 1H), 3.87 (td, J = 11.6, 2.8 Hz, 1H), 3.58 (tt, J = 7.4, 3.8 Hz, | |
| | 1H), 3.20 (td, J = 12.1, 3.6 Hz, 1H), 3.10 (dd, J = 12.8, 10.4 Hz, 1H), 2.67 (s, | |
| | 3H), 2.64 (s, 3H), 1.09 (td, J = 4.7, 2.9 Hz, 2H), 1.06-0.94 (m, 2H). | |
| 76 | ¹H NMR (400 MHz, DMSO-d6) δ ppm 7.84 (s, 1H), 7.72 (t, J = 7.9 Hz, 1H), | 496.2 |
| | 7.64 (dd, J = 9.8, 2.0 Hz, 1H), 7.56-7.38 (m, 2H), 4.73 (d, J = 13.2 Hz, 1H), | |
| | 4.61 (d, J = 13.3 Hz, 1H), 4.51 (dd, J = 10.5, 2.7 Hz, 1H), 4.01 (d, J = 11.5 | |
| | Hz, 1H), 3.76-3.60 (m, 2H), 3.28-3.15 (m, 2H), 2.65 (s, 3H), 2.51 (s, 3H), | |
| | 1.08-0.98 (m, 2H), 0.98-0.84 (m, 2H). | |
| 77 | ¹H NMR (400 MHz, CD2Cl2) δ ppm 7.59 (t, J = 7.9 Hz, 1H), 7.52 (s, 1H), | 479.1 |
| | 7.46 (s, 1H), 7.29 (dd, J = 8.4, 2.1 Hz, 1H), 7.24 (dd, J = 9.3, 2.0 Hz, 1H), | |
| | 6.97 (s, 1H), 4.63 (dd, J = 10.3, 2.8 Hz, 1H), 4.44-4.27 (m, 1H), 4.20-4.12 | |
| | (m, 1H), 4.12-4.05 (m, 1H), 3.85 (td, J = 11.5, 2.8 Hz, 1H), 3.57 (dq, J = | |
| | 7.4, 3.7 Hz, 1H), 3.15 (td, J = 12.1, 3.7 Hz, 1H), 3.05 (dd, J = 12.8, 10.3 Hz, | |
| | 1H), 2.65 (s, 3H), 2.56 (s, 3H), 1.14-1.04 (m, 2H), 1.02-0.94 (m, 2H). | |
| 78 | ¹H NMR (400 MHz, CD2Cl2) δ ppm 7.59 (t, J = 7.8 Hz, 1H), 7.52 (s, 1H), | 479.2 |
| | 7.46 (s, 1H), 7.29 (dd, J = 8.9, 1.6 Hz, 1H), 7.24 (dd, J = 9.5, 2.0 Hz, 1H), | |
| | 6.97 (s, 1H), 4.63 (dd, J = 10.3, 2.8 Hz, 1H), 4.38 (d, J = 12.8 Hz, 1H), 4.15 | |
| | (d, J = 12.9 Hz, 1H), 4.09 (d, J = 12.0 Hz, 1H), 3.85 (td, J = 11.5, 2.8 Hz, | |
| | 1H), 3.57 (tt, J = 7.2, 3.7 Hz, 1H), 3.15 (td, J = 12.0, 3.5 Hz, 1H), 3.05 | |
| | (dd, J = 12.8, 10.3 Hz, 1H), 2.65 (s, 3H), 2.56 (s, 3H), 1.14-1.03 (m, 2H), | |
| | 1.02-0.92 (m, 2H). | |
| 79 | ¹H NMR (400 MHz, CD2Cl2) δ ppm 7.59 (t, J = 7.8 Hz, 1H), 7.52 (s, 1H), | 496.2 |
| | 7.46 (s, 1H), 7.29 (dd, J = 8.9, 1.6 Hz, 1H), 7.24 (dd, J = 9.5, 2.0 Hz, 1H), | |
| | 6.97 (s, 1H), 4.63 (dd, J = 10.3, 2.8 Hz, 1H), 4.38 (d, J = 12.8 Hz, 1H), 4.15 | |
| | (d, J = 12.9 Hz, 1H), 4.09 (d, J = 12.0 Hz, 1H), 3.85 (td, J = 11.5, 2.8 Hz, 1H), | |
| | 3.57 (tt, J = 7.2, 3.7 Hz, 1H), 3.15 (td, J = 12.0, 3.5 Hz, 1H), 3.05 (dd, J = | |
| | 12.8, 10.3 Hz, 1H), 2.65 (s, 3H), 2.56 (s, 3H), 1.14-1.03 (m, 2H), | |
| | 1.02-0.92 (m, 2H). | |
| 80 | ¹H NMR (400 MHz, CD2Cl2) δ ppm 9.58 (d, J = 1.6 Hz, 1H), 8.78 (dd, J = | 496.2 |
| | 8.1, 2.0 Hz, 1H), 7.81 (d, J = 8.2 Hz, 1H), 7.54 (s, 1H), 7.48 (s, 1H), 7.01 (s, | |
| | 1H), 4.64 (dd, J = 10.4, 2.8 Hz, 1H), 4.44 (dd, J = 12.5, 2.8 Hz, 1H), 4.22 | |
| | (d, J = 12.7 Hz, 1H), 4.11 (d, J = 3.4 Hz, 1H), 3.87 (td, J = 11.6, 2.9 Hz, 1H), | |
| | 3.58 (tt, J = 7.5, 3.8 Hz, 1H), 3.20 (td, J = 12.1, 3.6 Hz, 1H), 3.10 (dd, J = | |
| | 12.8, 10.4 Hz, 1H), 2.67 (s, 3H), 2.64 (s, 3H), 1.14-1.03 (m, 2H), | |
| | 1.03-0.94 (m, 2H). | |
| 81 | ¹H NMR (400 MHz, Chloroform-d) δ ppm 8.72 (1H, s), 7.57 (1H, s), 7.48 | 470.2 |
| | (2H, s), 4.56 (1H, d, J = 11.2 Hz), 4.27-4.23 (1H, m), 3.85-3.77 (1H, m), 3.56 | |
| | (1H, m), 3.30-3.21 (1H, m), 2.78 (3H, s), 2.61 (6H, s), 2.31 (1H, d, J = 13.2 | |
| | Hz), 2.04-1.92 (3H, m), 1.12-1.06 (2H, m), 1.03-0.94 (2H, m). 19F NMR | |
| | (376 MHz, Chloroform-d) δ ppm-73.0 | |
| 82 | ¹H NMR (400 MHz, CD2Cl2) δ ppm 9.70 (s, 1H), 8.91 (d, J = 8.4 Hz, 1H), | 483.10 |
| | 7.88 (d, J = 8.2 Hz, 1H), 7.33 (d, J = 4.5 Hz, 1H), 6.65 (s, 1H), 6.34 (d, J = 7.2 | |
| | Hz, 1H), 5.11-4.99 (m, 1H), 4.90 (d, J = 13.5 Hz, 1H), 4.42 (d, J = 8.7 Hz, | |
| | 1H), 4.20 (d, J = 12.1 Hz, 1H), 3.82 (t, J = 11.2 Hz, 1H), 3.38-3.26 (m, 1H), | |
| | 3.06 (dd, J = 13.7, 9.8 Hz, 1H), 2.72 (s, 3H), 2.67 (s, 3H). one exchangeable | |
| | proton is not visible. 19F NMR (376 MHz, CD2Cl2) δ-68.40. | |
| 83 | ¹H NMR (400 MHz, CD2Cl2) δ ppm 8.47 (s, 1H), 7.55 (dd, J = 8.9, 5.9 Hz, | 448.2 |
| | 1H), 7.34 (dd, J = 8.8, 5.9 Hz, 1H), 7.28 (s, 1H), 7.20 (s, 1H), 5.02 (dd, J = | |
| | 13.6, 0.8 Hz, 1H), 4.85 (d, J = 13.6 Hz, 1H), 4.56 (d, J = 8.3 Hz, 1H), 4.18 | |
| | (dd, J = 11.6, 2.5 Hz, 1H), 3.87-3.79 (m, 1H), 3.36-3.25 (m, 1H), 3.03 | |
| | (dd, J = 13.4, 10.6 Hz, 1H), 2.68 (s, 3H), 2.57 (s, 3H), 2.55 (s, 3H). 19F NMR | |
| | (376 MHz, CD2Cl2) δ ppm-115.09 (s), -122.08 (s). | |
| 84 | ¹H NMR (400 MHz, DMSO-d6) δ ppm 8.87 (1H, s), 7.85 (1H, s), 7.66-7.72 | 482.2 |
| | (2H, m), 7.37-7.40 (2H, m), 7.25 (1H, t, J = 8.4 Hz), 4.48 (1H, d, J = 11.0 | |
| | Hz), 4.08 (1H, d, J = 11.2 Hz), 3.61-3.71 (2H, m), 2.74 (3H, s), 2.21 (1H, d, | |
| | J = 13.0 Hz), 1.85-1.97 (3H, m), 0.97 (2H, d, J = 4.2 Hz), 0.89 (2H, d, J = 7.3 | |
| | Hz). | |
| 85 | ¹H NMR (400 MHz, CD2Cl2) δ ppm 8.41 (s, 2H), 8.39 (s, 1H), 7.23 (s, 1H), | 482.2 |
| | 7.14 (d, J = 4.0 Hz, 1H), 4.56 (dd, J = 11.3, 1.1 Hz, 1H), 4.39-4.32 (m, 1H), | |
| | 3.90-3.79 (m, 1H), 3.64-3.51 (m, 1H), 2.81 (s, 3H), 2.79 (s, 3H), 2.52 (s, | |
| | 3H), 2.48-2.40 (m, 1H), 2.24-2.13 (m, 2H), 2.01-1.88 (m, 1H). 19F | |
| | NMR (376 MHz, CD2Cl2) δ ppm-113.77 (s), -113.80 (s). | |
| 86 | ¹H NMR (400 MHz, CD2Cl2) δ ppm 8.42 (d, J = 5.0 Hz, 1H), 8.35 (d, J = 8.2 | 483.10 |
| | Hz, 2H), 7.23 (s, 1H), 7.14 (d, J = 4.9 Hz, 1H), 4.69-4.57 (m, 2H), 4.40-4.33 | |
| | (m, 1H), 3.99-3.89 (m, 1H), 2.83 (s, 3H), 2.82 (s, 3H), 2.52 (s, 3H), | |
| | 2.34-2.24 (m, 1H), 2.23-2.16 (m, 1H), 2.12-2.01 (m, 1H), 2.01-1.93 (m, | |
| | 1H). 19F NMR (376 MHz, CD2Cl2) δ ppm-113.54 (s), -113.56 (s). | |
| 87 | ¹H NMR (400 MHz, Chloroform-d): δ ppm 7.54 (1H, s), 7.48 (2H, s), 4.55 | 484.2 |
| | (1H, d, J = 11.2 Hz), 4.25 (1H, d, J = 11.4 Hz), 3.78-3.84 (1H, m), 3.53-3.59 | |
| | (1H, m), 3.22 (1H, s), 2.73 (6H, d, J = 2.2 Hz), 2.61 (6H, s), 2.30 (1H, d, J = | |
| | 13.1 Hz), 1.95-2.02 (3H, m), 1.10 (2H, t, J = 3.5 Hz), 0.99 (2H, t, J = 6.7 Hz). | |
| 88 | ¹H NMR (400 MHz, CD2Cl2) δ ppm 8.47 (d, J = 4.9 Hz, 1H), 7.55 (dd, J = | 462.2 |
| | 8.9, 5.9 Hz, 1H), 7.34 (dd, J = 8.8, 5.9 Hz, 1H), 7.27 (s, 1H), 7.20 (d, J = 4.4 | |
| | Hz, 1H), 5.02 (d, J = 13.5 Hz, 1H), 4.85 (d, J = 13.4 Hz, 1H), 4.56 (dd, J = | |
| | 10.3, 2.0 Hz, 1H), 4.18 (dd, J = 11.5, 2.4 Hz, 1H), 3.83 (td, J = 11.7, 2.7 Hz, | |
| | 1H), 3.31 (td, J = 13.4, 3.5 Hz, 1H), 3.03 (dd, J = 13.4, 10.6 Hz, 1H), 2.68 (s, | |
| | 3H), 2.57 (s, 3H), 2.55 (s, 3H). 19F NMR (376 MHz, CD2Cl2) δ ppm-115.10 | |
| | (s), -122.08 (s). | |
| 89 | ¹H NMR (400 MHz, Chloroform-d) δ ppm 7.62 (t, J = 7.8 Hz, 1H), 7.48 (s, | 468.20 |
| | 1H), 7.42 (s, 1H), 7.31 (d, J = 8.5 Hz, 1H), 7.26 (d, J = 9.8 Hz, 1H), 4.84 | |
| | (d, J = 13.4 Hz, 1H), 4.77 (d, J = 13.5 Hz, 1H), 3.66-3.50 (m, 1H), 3.41 | |
| | (d, J = 11.9 Hz, 1H), 3.35-3.08 (m, 2H), 3.10-2.95 (m, 1H), 2.65 (s, 3H), | |
| | 2.54 (d, J = 2.5 Hz, 3H), 2.36 (t, J = 11.9 Hz, 1H), 2.16 (s, 3H), 1.08 (q, J = | |
| | 3.5 Hz, 2H), 0.98 (d, J = 6.8 Hz, 2H). | |
| 90 | ¹H NMR (400 MHz, Chloroform-d) δ ppm 7.67-7.58 (m, 1H), 7.48 (s, 1H), | 488.20 |
| | 7.42 (s, 1H), 7.31 (d, J = 8.4 Hz, 1H), 7.26 (d, J = 9.8 Hz, 1H), 4.91-4.80 | |
| | (m, 1H), 4.81-4.68 (m, 1H), 3.57 (tq, J = 7.5, 4.1 Hz, 1H), 3.48-3.36 (m, | |
| | 1H), 3.32-3.20 (m, 1H), 3.20-3.11 (m, 1H), 3.10-2.97 (m, 1H), 2.65 (s, | |
| | 3H), 2.54 (d, J = 2.8 Hz, 3H), 2.42-2.31 (m, 1H), 2.16 (s, 3H), 1.14-1.05 | |
| | (m, 2H), 1.02-0.92 (m, 2H). | |
| 91 | ¹H NMR (400 MHz, CD2Cl2δ ppm 8.81 (s, 1H), 8.41 (s, 1H), 7.50-7.45 (m, | 482.20 |
| | 1H), 7.43-7.37 (m, 1H), 7.23 (s, 1H), 7.13 (d, J = 4.6 Hz, 1H), 4.55 (d, J = | |
| | 11.5 Hz, 1H), 4.34 (dd, J = 10.6, 3.8 Hz, 1H), 3.84 (td, J = 11.7, 3.2 Hz, 1H), | |
| | 3.66-3.57 (m, 1H), 2.86 (s, 3H), 2.51 (s, 3H), 2.47-2.40 (m, 1H), 2.25-2.13 | |
| | (m, 2H), 2.01-1.90 (m, 1H). 19F NMR (376 MHz, CD2Cl2) | |
| | δ ppm-133.01 (s), -138.66 (s). | |
| 92 | ¹H NMR (400 MHz, CD2Cl2) δ ppm 8.41 (d, J = 5.1 Hz, 1H), 7.51-7.44 (m, | 482.20 |
| | 1H), 7.44-7.36 (m, 1H), 7.22 (s, 1H), 7.13 (d, J = 4.7 Hz, 1H), 4.54 (dd, J = | |
| | 11.6, 1.2 Hz, 1H), 4.37-4.30 (m, 1H), 3.88-3.77 (m, 1H), 3.63-3.52 (m, | |
| | 1H), 2.80 (s, 3H), 2.70 (s, 3H), 2.51 (s, 3H), 2.46-2.39 (m, 1H), 2.23-2.11 | |
| | (m, 2H), 2.00-1.89 (m, 1H) | |
| 93 | ¹H NMR (400 MHz, CDCl3) δ ppm 7.66 (dd, J = 14.9, 8.2 Hz, 1H), 7.52 (d, | 477.1 |
| | J = 6.9 Hz, 2H), 7.06-6.92 (m, 2H), 6.90 (s, 1H), 5.06 (t, J = 3.6 Hz, 1H), 4.34 | |
| | (dd, J = 12.9, 3.3 Hz, 1H), 4.07-3.87 (m, 2H), 3.62 (dd, J = 12.9, 3.8 Hz, 1H), | |
| | 3.51 (ddd, J = 11.0, 7.4, 3.8 Hz, 1H), 3.06 (dt, J = 22.0, 11.0 Hz, 1H), 2.63 (d, | |
| | J = 32.2 Hz, 6H), 1.26 (d, J = 6.3 Hz, 3H), 1.07-1.00 (m, 2H), 0.98-0.88 (m, | |
| | 2H). | |
| 94 | ¹H NMR (400 MHz, Chloroform-d) δ ppm 8.48-8.46 (1H, m), 7.82 (1H, s), | 446.2 |
| | 7.71-7.67 (1H, m), 7.33 (2H, m), 7.08-7.03 (1H, m), 7.02-6.96 (1H, m), | |
| | 4.96-4.92 (1H, m), 3.98 (2H, m), 3.56-3.53 (1H, m), 2.78 (3H, s), 2.70 (3H, s), | |
| | 2.66-2.60 (4H, m), 2.30-2.17 (3H, m). | |
| 95 | ¹H NMR (400 MHz, Chloroform-d) δ ppm 8.48-8.46 (1H, m), 7.82 (1H, s), | 446.2 |
| | 7.71-7.67 (1H, m), 7.33 (2H, m), 7.08-7.03 (1H, m), 7.02-6.96 (1H, m), | |
| | 4.96-4.92 (1H, m), 3.98 (2H, m), 3.56-3.53 (1H, m), 2.78 (3H, s), 2.70 (3H, s), | |
| | 2.66-2.60 (4H, m), 2.30-2.17 (3H, m). | |
| 96 | ¹H NMR (CHCl3-d, 400 MHz): δH 8.45 (1H, d, J = 5.1 Hz), 7.71 (1H, s), | 463.1 |
| | 7.66 (1H, s), 7.50 (1H, t, J = 7.9 Hz), 7.34 (1H, d, J = 8.4 Hz), 7.30 (1H, s), | |
| | 7.21 (1H, s), 7.11 (1H, s), 4.53 (1H, d, J = 11.3 Hz), 4.36 (1H, d, J = 11.5 | |
| | Hz), 3.82 (1H, t, J = 11.5 Hz), 3.37 (1H, m), 2.71 (3H, s), 2.55 (3H, s), 2.42 | |
| | (3H, s), 2.37 (1H, s), 2.05-2.10 (2H, m), 1.80 (1H, d, J = 12.3 Hz). | |
| 97 | | 478.0 |
| 98 | ¹H NMR (400 MHz, CDCl3) δ ppm 7.66 (dd, J = 14.9, 8.2 Hz, 1H), 7.52 (d, | 477.1 |
| | J = 6.9 Hz, 2H), 7.06-6.92 (m, 2H), 6.90 (s, 1H), 5.06 (t, J = 3.6 Hz, 1H), 4.34 | |
| | (dd, J = 12.9, 3.3 Hz, 1H), 4.07-3.87 (m, 2H), 3.62 (dd, J = 12.9, 3.8 Hz, 1H), | |
| | 3.51 (ddd, J = 11.0, 7.4, 3.8 Hz, 1H), 3.06 (dt, J = 22.0, 11.0 Hz, 1H), 2.63 (d, | |
| | J = 32.2 Hz, 6H), 1.26 (d, J = 6.3 Hz, 3H), 1.07-1.00 (m, 2H), 0.98-0.88 (m, | |
| | 2H). | |
| 99 | ¹H NMR (400 MHz, CDCl3) δ ppm 8.38 (s, 1H), 7.66-7.36 (m, 2H), 5.06 (s, | 486.0 |
| | 1H), 4.70 (dt, J = 100.5, 50.4 Hz, 2H), 4.05-3.68 (m, 2H), 3.49 (dq, J = 7.3, | |
| | 3.8 Hz, 1H), 3.27 (d, J = 87.1 Hz, 1H), 2.70 (s, 3H), 2.59 (d, J = 10.7 Hz, 6H), | |
| | 1.24 (s, 3H), 1.02 (dd, J = 8.4, 4.9 Hz, 2H), 0.93 (t, J = 16.8 Hz, 2H). | |
| 100 | ¹H NMR (400 MHz, CDCl3) δ ppm 8.38 (s, 1H), 7.54 (d, J = 8.6 Hz, 2H), | 486.0 |
| | 5.06 (d, J = 44.1 Hz, 2H), 4.57 (dd, J = 10.9, 2.4 Hz, 1H), 3.80 (ddd, J = 10.6, | |
| | 6.3, 2.5 Hz, 1H), 3.65-3.52 (m, 1H), 3.05 (s, 1H), 2.81 (dd, J = 13.2, 10.8 Hz, | |
| | 1H), 2.71 (s, 3H), 2.58 (s, 6H), 1.33 (d, J = 4.2 Hz, 3H), 1.13 (s, 2H), 0.99 | |
| | (dd, J = 27.6, 6.8 Hz, 2H). | |
| 101 | ¹H NMR (400 MHz, CDCl3) δ ppm 8.75 (d, J = 7.1 Hz, 1H), 7.56 (d, J = 3.9 | 471.0 |
| | Hz, 1H), 7.47 (d, J = 15.4 Hz, 1H), 4.86-4.13 (m, 2H), 3.88-3.75 (m, 1H), | |
| | 3.51 (t, J = 33.6 Hz, 2H), 2.81 (d, J = 5.2 Hz, 3H), 2.59 (s, 7H), 2.32 (d, J = | |
| | 13.7 Hz, 1H), 2.08 (dd, J = 20.6, 8.9 Hz, 2H), 1.03 (d, J = 5.0 Hz, 4H). | |
| 102 | ¹H NMR (400 MHz, CDCl3) δ ppm 8.75 (d, J = 7.1 Hz, 1H), 7.56 (d, J = 3.9 | 471.0 |
| | Hz, 1H), 7.47 (d, J = 15.4 Hz, 1H), 4.86-4.13 (m, 2H), 3.88-3.75 (m, 1H), | |
| | 3.51 (t, J = 33.6 Hz, 2H), 2.81 (d, J = 5.2 Hz, 3H), 2.59 (s, 7H), 2.32 (d, J = | |
| | 13.7 Hz, 1H), 2.08 (dd, J = 20.6, 8.9 Hz, 2H), 1.03 (d, J = 5.0 Hz, 4H). | |
| 103 | ¹H NMR (400 MHz, CDCl3) δ ppm 7.46 (s, 2H), 5.05 (t, J = 3.5 Hz, 1H), 4.86 | 500.0 |
| | (s, 1H), 4.60 (s, 1H), 3.90 (s, 2H), 3.49 (ddd, J = 11.1, 7.3, 3.8 Hz, 1H), 3.15 | |
| | (s, 1H), 2.70 (d, J = 14.3 Hz, 3H), 2.63 (s, 3H), 2.60 (s, 6H), 1.26-1.22 (m, | |
| | 3H), 1.05-0.99 (m, 2H), 0.95 (d, J = 14.3 Hz, 2H). | |
| 104 | ¹H NMR (400 MHz, CDCl3) δ ppm 7.46 (s, 2H), 5.05 (t, J = 3.4 Hz, 1H), 4.87 | 500.0 |
| | (s, 1H), 4.60 (s, 1H), 3.89 (s, 2H), 3.49 (ddd, J = 11.1, 7.3, 3.8 Hz, 1H), 3.14 | |
| | (s, 1H), 2.66 (d, J = 10.5 Hz, 3H), 2.63 (s, 3H), 2.60 (s, 6H), 1.23 (d, J = 6.2 | |
| | Hz, 3H), 1.05-0.99 (m, 2H), 0.94 (s, 2H). | |
| 105 | ¹H NMR (400 MHz, CDCl3) δ ppm 7.58-7.50 (m, 2H), 5.09-4.89 (m, 2H), | 500.0 |
| | 4.57 (dd, J = 10.9, 2.6 Hz, 1H), 3.80 (ddd, J = 10.6, 6.3, 2.6 Hz, 1H), 3.58 (tt, | |
| | J = 7.3, 3.8 Hz, 1H), 3.07-2.99 (m, 1H), 2.79 (dd, J = 13.3, 10.7 Hz, 1H), 2.68 | |
| | (s, 3H), 2.63 (s, 3H), 2.58 (s, 6H), 1.32 (d, J = 6.2 Hz, 3H), 1.13 (s, 2H), | |
| | 1.05-0.98 (m, 2H). | |
| 106 | ¹H NMR (400 MHz, CDCl3) δ ppm 7.57-7.51 (m, 2H), 4.98 (d, J = 46.1 Hz, | 500.0 |
| | 2H), 4.57 (dd, J = 10.9, 2.6 Hz, 1H), 3.80 (ddd, J = 10.5, 6.2, 2.5 Hz, 1H), | |
| | 3.58 (tt, J = 7.3, 3.8 Hz, 1H), 3.07-2.98 (m, 1H), 2.79 (dd, J = 13.3, 10.7 Hz, | |
| | 1H), 2.68 (s, 3H), 2.63 (s, 3H), 2.58 (s, 6H), 1.32 (d, J = 6.1 Hz, 3H), 1.10 (d, | |
| | J = 16.9 Hz, 2H), 1.05-0.98 (m, 2H). | |
| 107 | ¹H NMR (400 MHz, CDCl3) δ 7.86 (d, J = 6.7 Hz, 1H), 7.63 (d, J = 7.7 Hz, | 513.0 |
| | 1H), 7.52 (s, 1H), 7.50 (s, 1H), 7.47 (s, 1H), 4.88 (d, J = 4.6 Hz, 1H), 3.94 (t, | |
| | J = 5.2 Hz, 2H), 3.77-3.67 (m, 1H), 3.61-3.52 (m, 1H), 2.87 (s, 3H), 2.76 | |
| | (s, 1H), 2.74 (s, 3H), 1.69 (s, 3H), 1.11 (d, J = 3.5 Hz, 2H), 1.00 (d, J = 5.3 | |
| | Hz, 2H). | |
| 108 | ¹H NMR (400 MHz, CDCl3) δ 7.86 (t, J = 7.2 Hz, 1H), 7.63 (d, J = 8.0 Hz, | 513.0 |
| | 1H), 7.57-7.47 (m, 3H), 4.27 (dt, J = 5.8, 3.1 Hz, 1H), 3.81 (ddd, J = 18.8, | |
| | 12.5, 6.7 Hz, 1H), 3.61-3.50 (m, 2H), 2.85 (d, J = 10.7 Hz, 3H), 2.74 (s, | |
| | 3H), 2.45 (d, J = 13.3 Hz, 1H), 2.29-2.12 (m, 3H), 1.79 (s, 1H), 1.15-1.05 | |
| | (m, 2H), 0.99 (dt, J = 12.5, 6.2 Hz, 2H). | |
| 109 | 1H NMR (CHCl3-d, 400 MHz): δH 8.00 (1H, dd, J = 8.5, 4.0 Hz), 7.59 | 478.2 |
| | (1H, t, J = 7.9 Hz), 7.50 (2H, d, J = 6.7 Hz), 7.40 (2H, t, J = 7.4 Hz), 7.32 | |
| | (1H, d, J = 9.6 Hz), 4.60 (1H, d, J = 11.4 Hz), 3.84 (1H, dd, J = 11.0, 6.1 | |
| | Hz), 3.51-3.56 (2H, m), 2.86 (3H, s), 2.39 (1H, d, J = 13.3 Hz), 2.10-2.23 | |
| | (2H, m), 1.87 (1H, q, J = 12.1 Hz), 1.32 (3H, d, J = 6.1 Hz), 1.09 (2H, t, J = | |
| | 3.5 Hz), 0.98 (2H, t, J = 6.7 Hz). | |
| 110 | ¹H NMR (400 MHz, Methylene Chloride-d2) δ 8.80 (s, 1H), 7.75 (td, J = 8.2, | 463.1 |
| | 6.4 Hz, 1H), 7.50 (s, 1H), 7.42 (s, 1H), 7.12 (td, J = 7.8, 2.0 Hz, 1H), 7.03 | |
| | (td, J = 9.6, 2.4 Hz, 1H), 4.60 (dd, J = 11.5, 2.1 Hz, 1H), 3.83 (dtd, J = 12.4, | |
| | 6.2, 2.0 Hz, 1H), 3.63-3.39 (m, 2H), 2.85 (s, 3H), 2.37 (ddt, J = 13.1, 4.0, | |
| | 2.1 Hz, 1H), 2.21 (ddt, J = 13.2, 4.0, 2.0 Hz, 1H), 2.03 (q, J = 11.8 Hz, 1H), | |
| | 1.76 (d, J = 12.3 Hz, 1H), 1.29 (d, J = 6.2 Hz, 3H), 1.12-1.02 (m, 2H), | |
| | 1.01-0.87 (m, 2H). | |
| 111 | 1H NMR (400 MHz, Methylene Chloride-d2) δ 8.00 (dd, J = 8.4, 3.9 Hz, 1H), | 462.2 |
| | 7.65 (td, J = 8.3, 6.3 Hz, 1H), 7.50 (s, 1H), 7.41 (d, J = 8.8 Hz, 2H), 7.15 | |
| | (td, J = 8.3, 2.5 Hz, 1H), 7.06 (td, J = 9.5, 2.4 Hz, 1H), 4.59 (dd, J = 11.5, 2.1 | |
| | Hz, 1H), 3.83 (dqt, J = 12.4, 6.2, 2.6 Hz, 1H), 3.55 (dq, J = 7.3, 3.7 Hz, 1H), | |
| | 3.49 (dt, J = 12.2, 3.8 Hz, 1H), 2.80 (s, 3H), 2.36 (ddt, J = 13.1, 4.0, 2.0 Hz, | |
| | 1H), 2.20 (ddt, J = 13.1, 3.9, 2.0 Hz, 1H), 2.03 (dt, J = 13.3, 11.8 Hz, 1H), | |
| | 1.86-1.69 (m, 1H), 1.29 (d, J = 6.2 Hz, 3H), 1.12-1.02 (m, 2H), 1.01-0.84 | |
| | (m, 2H). | |
| 112 | 1H NMR (400 MHz, Methylene Chloride-d2) δ 8.00 (dd, J = 8.4, 3.9 Hz, 1H), | 462.2 |
| | 7.65 (td, J = 8.3, 6.3 Hz, 1H), 7.50 (s, 1H), 7.41 (d, J = 8.8 Hz, 2H), 7.15 | |
| | (td, J = 8.3, 2.5 Hz, 1H), 7.06 (td, J = 9.5, 2.4 Hz, 1H), 4.59 (dd, J = 11.5, 2.1 | |
| | Hz, 1H), 3.83 (dqt, J = 12.4, 6.2, 2.6 Hz, 1H), 3.55 (dq, J = 7.3, 3.7 Hz, 1H), | |
| | 3.49 (dt, J = 12.2, 3.8 Hz, 1H), 2.80 (s, 3H), 2.36 (ddt, J = 13.1, 4.0, 2.0 Hz, | |
| | 1H), 2.20 (ddt, J = 13.1, 3.9, 2.0 Hz, 1H), 2.03 (dt, J = 13.3, 11.8 Hz, 1H), | |
| | 1.86-1.69 (m, 1H), 1.29 (d, J = 6.2 Hz, 3H), 1.12-1.02 (m, 2H), 1.01-0.84 | |
| | (m, 2H). | |
| 113 | ¹H NMR (400 MHz, CDCl3) δ 7.72 (dd, J = 14.8, 8.1 Hz, 1H), 7.51 (d, J = 9.1 | 478.1 |
| | Hz, 2H), 7.11-6.89 (m, 2H), 5.06 (s, 1H), 4.75 (d, J = 69.4 Hz, 2H), 3.86 (d, | |
| | J = 64.9 Hz, 2H), 3.49 (s, 1H), 3.21 (s, 1H), 2.72 (s, 3H), 2.60 (s, 3H), 1.25 (d, | |
| | J = 6.3 Hz, 3H), 1.01 (s, 2H), 0.93 (dd, J = 6.7, 3.1 Hz, 2H). | |
| 114 | ¹H NMR (400 MHz, CDCl3) δ ppm 8.42 (s, 1H), 7.66-7.58(m, 1H), 7.51(d, | 464.0 |
| | J = 8.2 Hz, 2H), 7.08-6.94(m, 2H), 6.90(s, 1H), 5.07 (t, J = 3.7 Hz, 1H), 4.38 | |
| | (dd, J = 12.8, 3.4 Hz, 1H), 4.03-3.89 (m, 2H), 3.66 (dd, J = 13.0, 3.9 Hz, | |
| | 1H), 3.51 (td, J = 7.3, 3.7 Hz, 1H), 3.12 (dd, J = 12.5, 8.6 Hz, 1H), 2.70 (s, | |
| | 3H), 1.27 (d, J = 6.3 Hz, 3H), 1.07-1.01 (m, 2H), 0.99-0.91 (m, 2H). | |
| 115 | ¹H NMR (400 MHz, CDCl3) δ 8.41 (s, 1H), 7.69-7.66 (m, 1H), 7.53 (s, 2H), | 464.0 |
| | 7.08-6.96 (m, 2H), 5.04-4.99 (m, 2H), 4.60 (d, J = 10.0 Hz, 1H), 3.85-3.81 | |
| | (m, 1H), 3.60-3.55 (m, 1H), 3.01 (s, J = 10.8 Hz, 1H), 2.86 (t, J = 10.8 Hz, | |
| | 1H), 2.72 (s, 3H), 1.32 (dd, J = 6.0 Hz, 3H), 1.14-1.06 (m, 2H), 1.04-0.99 | |
| | (m, 2H). | |
| 116 | 1H NMR (CHCl3-d, 400 MHz): δH 8.68 (1H, s), 7.50 (3H, s), 4.88 (2H, s), | 430.3 |
| | 4.61 (1H, d, J = 11.3 Hz), 3.85 (1H, d, J = 8.9 Hz), 3.51 (3H, d, J = 17.8 Hz), | |
| | 3.27 (1H, t, J = 12.3 Hz), 2.95 (2H, d, J = 16.4 Hz), 2.76 (3H, s), 2.30 (1H, | |
| | d, J = 13.0 Hz), 2.10 (1H, d, J = 13.2 Hz), 1.93 (1H, t, J = 12.2 Hz), 1.74 (3H, | |
| | s), 1.68-1.59 (1H, m), 1.33 (3H, d, J = 6.2 Hz), 1.10 (2H, s), 0.98 (2H, d, J = | |
| | 7.1 Hz) | |
| 117 | ¹H NMR (400 MHz, CDCl3) δ ppm 8.41 (s, 1H), 7.62 (s, 1H), 7.53 (d, J = 5.8 | 481.0 |
| | Hz, 2H), 7.30 (dd, J = 15.3, 7.4 Hz, 2H), 5.04 (s, 2H), 4.60 (d, J = 9.4 Hz, | |
| | 1H), 3.83 (s, 1H), 3.58 (s, 1H), 3.10 (dd, J = 13.4, 11.0 Hz, 1H), 2.86 (dd, J = | |
| | 13.4, 10.7 Hz, 1H), 2.74 (s, 3H), 1.33 (d, J = 6.2 Hz, 3H), 1.12 (dt, J = 8.3, 4.3 | |
| | Hz, 2H), 1.06-0.98 (m, 2H). | |
| 118 | ¹H NMR (400 MHz, CDCl3) δ ppm 8.41 (s, 1H), 7.62 (s, 1H), 7.58-7.50 (m, | 481.0 |
| | 2H), 7.38-7.27 (m, 2H), 5.04 (s, 2H), 4.60 (d, J = 9.1 Hz, 1H), 3.83 (s, 1H), | |
| | 3.58 (s, 1H), 3.09 (dd, J = 13.4, 11.0 Hz, 1H), 2.86 (dd, J = 13.3, 10.7 Hz, | |
| | 1H), 2.74 (s, 3H), 1.33 (d, J = 6.2 Hz, 3H), 1.18-1.07 (m, 2H), 1.06-0.95 (m, | |
| | 2H). | |
| 119 | ¹H NMR (400 MHz, CDCl3) δ ppm 7.66 (t, J = 7.8 Hz, 1H), 7.52 (s, 2H), | 494.0 |
| | 7.37-7.27 (m, 2H), 5.06 (s, 1H), 4.76 (d, J = 57.5 Hz, 2H), 3.86 (d, J = 65.7 | |
| | Hz, 2H), 3.50 (s, 1H), 3.21 (s, 1H), 2.72 (s, 3H), 2.59 (s, 3H), 1.25 (d, J = 6.2 | |
| | Hz, 3H), 1.02 (s, 2H), 0.95 (s, 2H). | |
| 120 | ¹H NMR (400 MHz, CDCl3) δ ppm 7.65 (t, J = 7.8 Hz, 1H), 7.53 (t, J = 4.1 | 494.0 |
| | Hz, 2H), 7.30 (t, J = 6.0 Hz, 1H), 7.24 (d, J = 2.0 Hz, 1H), 4.97 (s, 2H), 4.60 | |
| | (d, J = 8.7 Hz, 1H), 3.89-3.76 (m, 1H), 3.57 (ddd, J = 11.1, 7.3, 3.9 Hz, 1H), | |
| | 3.07 (dd, J = 13.3, 11.0 Hz, 1H), 2.84 (dd, J = 13.4, 10.7 Hz, 1H), 2.71 (s, | |
| | 3H), 2.59 (s, 3H), 1.32 (d, J = 6.2 Hz, 3H), 1.15-1.07 (m, 2H), 1.01 (dt, J = | |
| | 12.3, 6.3 Hz, 2H). | |
| 121 | ¹H NMR (400 MHz, CDCl3) δ 7.71 (dd, J = 14.9, 7.8 Hz, 1H), 7.54 (t, J = 4.8 | 478.1 |
| | Hz, 2H), 7.10-6.87 (m, 2H), 5.03 (d, J = 37.5 Hz, 2H), 4.60 (d, J = 8.5 Hz, | |
| | 1H), 3.92-3.72 (m, 1H), 3.57 (ddd, J = 10.9, 7.2, 3.7 Hz, 1H), 3.08 (dd, J = | |
| | 13.4, 11.0 Hz, 1H), 2.84 (dd, J = 13.3, 10.7 Hz, 1H), 2.71 (s, 3H), 2.59 (s, | |
| | 3H), 1.33 (d, J = 6.2 Hz, 3H), 1.16-1.08 (m, 2H), 1.01 (q, J = 6.8 Hz, 2H). | |
| 122 | ¹H NMR (400 MHz, CDCl3) δ 7.71 (dd, J = 15.0, 7.9 Hz, 1H), 7.54 (t, J = 5.2 | 478.1 |
| | Hz, 2H), 7.07-6.93 (m, 2H), 5.03 (d, J = 37.1 Hz, 3H), 4.60 (d, J = 8.6 Hz, | |
| | 1H), 3.91-3.74 (m, 1H), 3.65-3.47 (m, 1H), 3.08 (dd, J = 13.3, 11.0 Hz, 1H), | |
| | 2.84 (dd, J = 13.4, 10.7 Hz, 1H), 2.71 (s, 3H), 2.59 (s, 3H), 1.33 (d, J = 6.2 | |
| | Hz, 3H), 1.16-1.07 (m, 2H), 1.06-0.97 (m, 2H). | |
| 123 | ¹H NMR (DMSO-d6, 400 MHz): δH 8.73 (1H, d, J = 8.5 Hz), 7.71 (1H, s), | 443.6 |
| | 7.53 (1H, d, J = 8.5 Hz), 7.36 (1H, s), 4.48 (1H, d, J = 11.2 Hz), 4.09-4.05 | |
| | (1H, m), 3.66 (2H, d, J = 13.7 Hz), 3.27 (2H, s), 2.69 (3H, s), 2.17 (7H, s), | |
| | 1.96 (1H, s), 1.86 (2H, br s), 1.77 (1H, s), 1.22 (1H, s), 0.98 (2H, s), 0.89 (6H, | |
| | d, J = 6.8 Hz) | |
| 124 | ¹H NMR (CHCl3-d, 400 MHz): δH 8.34 (1H, d, J = 8.5 Hz), 7.49 (1.5H, | 392.3 |
| | d, J = 4.2 Hz), 7.42 (0.5H, d, J = 3.7 Hz), 7.38 (1H, d, J = 8.4 Hz), 4.59 (1H, | |
| | d, J = 11.5 Hz), 3.84-3.77 (2H, m), 3.56-3.50 (1H, m), 3.46-3.40 (1H, m), | |
| | 2.80 (3H, s), 2.35 (1H, d, J = 13.3 Hz), 2.17 (1H, d, J = 14.5 Hz), 2.09 (1H, | |
| | q, J = 12.2 Hz), 1.81 (1H, q, J = 12.2 Hz), 1.40 (6H, d, J = 6.8 Hz), 1.31 (3H, | |
| | d, J = 6.2 Hz), 1.25-1.21 (1H, m), 1.08 (2H, s), 0.96 (2H, t, J = 6.7 Hz) | |
| 125 | 1H NMR (400 MHz, CDCl3) δ ppm 8.38 (s, 1H), 7.54 (d, J = 8.6 Hz, 2H), | 486.0 |
| | 5.06 (d, J = 44.1 Hz, 2H), 4.57 (dd, J = 10.9, 2.4 Hz, 1H), 3.80 (ddd, J = 10.6, | |
| | 6.3, 2.5 Hz, 1H), 3.65-3.52 (m, 1H), 3.05 (s, 1H), 2.81 (dd, J = 13.2, 10.8 Hz, | |
| | 1H), 2.71 (s, 3H), 2.58 (s, 6H), 1.33 (d, J = 4.2 Hz, 3H), 1.13 (s, 2H), 0.99 | |
| | (dd, J = 27.6, 6.8 Hz, 2H). | |
| 126 | 1H NMR (400 MHz, CDCl3) δ ppm 8.38 (s, 1H), 7.66-7.36 (m, 2H), 5.06 (s, | 486.0 |
| | 1H), 4.70 (dt, J = 100.5, 50.4 Hz, 2H), 4.05-3.68 (m, 2H), 3.49 (dq, J = 7.3, | |
| | 3.8 Hz, 1H), 3.27 (d, J = 87.1 Hz, 1H), 2.70 (s, 3H), 2.59 (d, J = 10.7 Hz, 6H), | |
| | 1.24 (s, 3H), 1.02 (dd, J = 8.4, 4.9 Hz, 2H), 0.93 (t, J = 16.8 Hz, 2H). | |
| 127 | 1H NMR (DMSO-d6, 400 MHz): δH 7.70 (1H, s), 7.36 (1H, s), 4.48 (1H, | 459.3 |
| | d, J = 11.2 Hz), 4.07 (1H, dd, J = 11.2, 4.1 Hz), 3.69-3.60 (2H, m), 3.36-3.33 | |
| | (1H, m), 2.71 (6H, s), 2.22 (1H, d, J = 12.9 Hz), 2.17 (6H, s), 1.97 (1H, d, J = | |
| | 13.1 Hz), 1.92-1.74 (3H, m), 1.00-0.97 (2H, m), 0.88 (8H, d, J = 6.7 Hz) | |
| 128 | 1H NMR (CHCl3-d, 400 MHz): δH 8.82 (1H, s), 7.69 (1H, t, J = 7.8 Hz), | 479.2 |
| | 7.52 (1H, s), 7.51 (1H, s), 7.37 (1H, d, J = 8.4 Hz), 7.30 (1H, dd, J = 9.5, | |
| | 1.9 Hz), 4.61 (1H, d, J = 11.3 Hz), 3.85 (1H, m), 3.51-3.61 (2H, m), 2.90 | |
| | (3H, s), 2.42 (1H, d, J = 13.3 Hz), 2.25 (1H, d, J = 13.3 Hz), 2.14 (1H, q, | |
| | J = 12.3 Hz), 1.86 (1H, q, J = 12.1 Hz), 1.33 (3H, d, J = 6.2 Hz), 1.09 (2H, | |
| | d, J = 3.9 Hz), 0.95-1.00 (2H, m). | |
| 129 | 1H NMR (CHCl3-d, 400 MHz): δH 8.82 (1H, s), 7.69 (1H, t, J = 7.8 Hz), | 479.2 |
| | 7.52 (1H, s), 7.51 (1H, s), 7.37 (1H, d, J = 8.4 Hz), 7.30 (1H, dd, J = 9.5, | |
| | 1.9 Hz), 4.61 (1H, d, J = 11.3 Hz), 3.85 (1H, m), 3.51-3.61 (2H, m), 2.90 | |
| | (3H, s), 2.42 (1H, d, J = 13.3 Hz), 2.25 (1H, d, J = 13.3 Hz), 2.14 (1H, q, | |
| | J = 12.3 Hz), 1.86 (1H, q, J = 12.1 Hz), 1.33 (3H, d, J = 6.2 Hz), 1.09 (2H, | |
| | d, J = 3.9 Hz), 0.95-1.00 (2H, m). | |
| 130 | 1H NMR (400 MHz, CD2Cl2) δ 8.81 (s, 1H), 8.41 (d, J = 4.9 Hz, 1H), | 468.100 |
| | 7.51-7.45 (m, 1H), 7.43-7.38 (m, 1H), 7.23 (s, 1H), 7.14 (d, J = 5.3 Hz, | |
| | 1H), 4.55 (dd, J = 11.0, 0.9 Hz, 1H), 4.34 (ddd, J = 5.2, 4.3, 1.5 Hz, 1H), | |
| | 3.84 (td, J = 11.7, 2.8 Hz, 1H), 3.66-3.57 (m, 1H), 2.86 (s, 3H), 2.52 (s, 3H), | |
| | 2.48-2.41 (m, 1H), 2.25-2.10 (m, 2H), 2.01-1.90 (m, 1H). 19F NMR | |
| | (376 MHz, CD2Cl2) δ-133.01 (s), -138.75 (s). | |
| 131 | 1H NMR (400 MHz, CD2Cl2) δ 8.81 (s, 1H), 8.41 (d, J = 4.9 Hz, 1H), | 468.100 |
| | 7.52-7.44 (m, 1H), 7.44-7.37 (m, 1H), 7.23 (s, 1H), 7.13 (d, J = 5.0 Hz, 1H), | |
| | 4.55 (dd, J = 11.1, 1.2 Hz, 1H), 4.34 (ddd, J = 11.4, 4.1, 1.0 Hz, 1H), 3.83 | |
| | (td, J = 11.6, 2.8 Hz, 1H), 3.66-3.57 (m, 1H), 2.86 (s, 3H), 2.52 (s, 3H), | |
| | 2.45 (dd, J = 7.4, 6.7 Hz, 1H), 2.25-2.11 (m, 2H), 2.02-1.90 (m, 1H). 19F | |
| | NMR (376 MHz, CD2Cl2) δ-133.00 (s), -138.75 (s). | |
| 132 | 1H NMR (400 MHz, CD2Cl2) δ 8.44-8.37 (m, 3H), 7.24 (s, 1H), 7.14 | 482.200 |
| | (d, J = 4.5 Hz, 1H), 4.56 (dd, J = 11.2, 1.2 Hz, 1H), 4.35 (ddd, J = 11.8, 4.3, | |
| | 1.5 Hz, 1H), 3.85 (td, J = 11.6, 3.3 Hz, 1H), 3.63-3.53 (m, 1H), 2.81 (s, 3H), | |
| | 2.79 (s, 3H), 2.52 (s, 3H), 2.48-2.41 (m, 1H), 2.25-2.11 (m, 2H), 2.01-1.89 | |
| | (m, 1H). 19F NMR (376 MHz, CD2Cl2) δ-113.77 (s), -113.80 (s). | |
| 133 | 1H NMR (400 MHz, CD2Cl2) δ 8.44-8.37 (m, 3H), 7.24 (s, 1H), 7.15 | 482.150 |
| | (d, J = 5.2 Hz, 1H), 4.56 (dd, J = 11.5, 1.7 Hz, 1H), 4.35 (ddd, J = 11.5, 4.1, | |
| | 1.6 Hz, 1H), 3.85 (td, J = 11.6, 3.1 Hz, 1H), 3.63-3.53 (m, 1H), 2.81 (s, 3H), | |
| | 2.79 (s, 3H), 2.52 (s, 3H), 2.48-2.41 (m, 1H), 2.25-2.10 (m, 2H), 2.00-1.89 | |
| | (m, 1H). 19F NMR (376 MHz, CD2Cl2) δ-113.77 (s), -113.79 (s). | |
| 134 | 1H NMR (400 MHz, CDCl3) δ 7.56 (s, 1H), 7.53 (s, 1H), 5.02-4.81 (m, 1H), | 517.2 |
| | 4.57 (dd, J = 11.5, 1.9 Hz, 1H), 4.34-4.24 (m, 2H), 3.90-3.73 (m, 1H), | |
| | 3.49-3.41 (m, 1H), 3.03-2.92 (m, 2H), 2.87-2.77 (m, 8H), 2.65 (s, 6H), | |
| | 2.41-2.35 (m, 1H), 2.22-2.08 (m, 3H). | |
| 135 | 1H NMR (400 MHz, CDCl3) δ 7.56 (s, 1H), 7.53 (s, 1H), 5.02-4.81 (m, 1H), | 517.2 |
| | 4.57 (dd, J = 11.5, 1.9 Hz, 1H), 4.34-4.24 (m, 2H), 3.90-3.73 (m, 1H), | |
| | 3.49-3.41 (m, 1H), 3.03-2.92 (m, 2H), 2.87-2.77 (m, 8H), 2.65 (s, 6H), | |
| | 2.41-2.35 (m, 1H), 2.22-2.08 (m, 3H). | |
| 136 | 1H NMR (400 MHz, CD2Cl2) δ 8.40 (d, J = 4.3 Hz, 1H), 7.50-7.44 (m, | 488.2 |
| | 1H), 7.43-7.37 (m, 1H), 7.22 (s, 1H), 7.13 (d, J = 4.5 Hz, 1H), 4.54 (dd, J = | |
| | 11.5, 1.6 Hz, 1H), 4.37-4.30 (m, 1H), 3.83 (td, J = 11.6, 3.0 Hz, 1H), 3.63-3.53 | |
| | (m, 1H), 2.51 (s, 3H), 2.46-2.40 (m, 1H), 2.24-2.10 (m, 2H), 2.00-1.89 | |
| | (m, 1H). Note: H/D isotopic signals for dimethyl group are seen at 2.79 | |
| | and 2.6δ ppm. 19F NMR (376 MHz, CD2Cl2) δ-132.77 (s), -138.83 (s). | |
| 137 | 1H NMR (400 MHz, CD2Cl2) δ 8.41 (d, J = 5.1 Hz, 1H), 7.50-7.46 (m, | 488.2 |
| | 1H), 7.42-7.37 (m, 1H), 7.23 (s, 1H), 7.14 (d, J = 5.0 Hz, 1H), 4.55 (dd, J = | |
| | 11.6, 1.1 Hz, 1H), 4.37-4.31 (m, 1H), 3.83 (td, J = 11.6, 2.9 Hz, 1H), | |
| | 3.63-3.54 (m, 1H), 2.52 (s, 3H), 2.43 (dd, J = 13.1, 1.9 Hz, 1H), 2.24-2.09 (m, | |
| | 2H), 2.00-1.89 (m, 1H). Note: H/D isotopic signals for dimethyl group are | |
| | seen at 2.79 and 2.6δ ppm. 19F NMR (376 MHz, CD2Cl2) δ-132.84 (s), | |
| | -138.82 (s). | |
| 138 | 1H NMR (500 MHz, CDCl3) δ 8.56 (d, J = 8.5 Hz, 2H), 7.81 (d, J = 8.5 Hz, | 495.2 |
| | 2H), 7.51 (s, 2H), 4.58 (dd, J = 11.4, 1.8 Hz, 1H), 4.28 (dd, J = 5.8, 3.3 Hz, | |
| | 1H), 3.88-3.79 (m, 1H), 3.61-3.46 (m, 2H), 2.89 (s, 3H), 2.82 (s, 3H), 2.46 | |
| | (d, J = 13.3 Hz, 1H), 2.28-2.17 (m, 3H), 1.11-1.06 (m, 2H), 1.01-0.94 | |
| | (m, 2H) | |
| 139 | 1H NMR (500 MHz, CDCl3) δ 8.55 (d, J = 8.1 Hz, 2H), 7.82 (d, J = 8.2 Hz, | 495.2 |
| | 2H), 7.49 (d, J = 19.1 Hz, 2H), 4.58 (dd, J = 11.4, 1.9 Hz, 1H), 4.28 (dd, J = | |
| | 6.0, 3.3 Hz, 1H), 3.91-3.76 (m, 1H), 3.61-3.49 (m, 2H), 2.86 (s, 3H), 2.80 | |
| | (s, 3H), 2.46 (d, J = 13.4 Hz, 1H), 2.27-2.16 (m, 3H), 1.13-1.04 (m, 2H), | |
| | 1.03-0.96 (m, 2H) | |
| 140 | 1H NMR (DMSO-d6, 400 MHz): δH 8.98 (1H, s), 7.72 (1H, s), 7.37 (1H, s), | 485.3 |
| | 4.54 (1H, d, J = 11.3 Hz), 3.77 (1H, s), 3.64 (1H, d, J = 6.1 Hz), 3.42 (1H, s), | |
| | 2.76 (3H, s), 2.61 (7H, s), 2.19 (1H, d, J = 13.3 Hz), 2.06 (1H, d, J = 12.6 Hz), | |
| | 1.88-1.81 (1H, m), 1.56 (1H, t, J = 12.1 Hz), 1.22-1.18 (4H, m), 0.99 (2H, s), | |
| | 0.93-0.88 (2H, m) | |
| 141 | 1H NMR (400 MHz, CDCl3) δ 8.1 (d, J = 2.4 Hz, 1H), 7.68 (dd, J = 2.8 Hz, J = | 486.1 |
| | 8.8 Hz, 1H), 6.74 (d, J = 8.4 Hz, 1H), 7.32-7.21 (m, 1H), 4.52 (dd, J = 2 Hz, | |
| | J = 11.2 Hz, 1H), 4.34-4.30 (m, 1H), 3.92(s, 3H), 3.86-3.80 (m, 1H), | |
| | 3.51-3.46 (m, 1H), 3.30 (d, J = m, 1H), 2.80 (s, 3H), 2.77 (s, 3H), | |
| | 2.64 (s, 6H), 2.32-2.29 (m, 1H), 2.20-2.04 (m, 3H). | |
| 142 | 1H NMR (400 MHz, CDCl3) δ 8.1 (d, J = 2.4 Hz, 1H), 7.85 (dd, J = 2.8 Hz, J = | 486.1 |
| | 8.8 Hz, 1H), 6.74 (d, J = 8.4 Hz, 1H), 7.32-7.21 (m, 1H), 4.52 (dd, J = 2 Hz, | |
| | J = 11.2 Hz, 1H), 4.34-4.30 (m, 1H), 3.92(s, 3H), 3.86-3.80 (m, 1H), | |
| | 3.51-3.46 (m, 1H), 3.30 (d, J = m, 1H), 2.80 (s, 3H), 2.77 (s, 3H), 2.64 (s, 6H), | |
| | 2.32-2.29 (m, 1H), 2.20-2.0 (m, 3H). | |
| 143 | 1H NMR (400 MHz, CDCl3) δ 8.06 (dd, J = 2 Hz, J = 5.2 Hz, 1H), 7.79 (dd, | 486.3 |
| | J = 2 Hz, J = 7.2 Hz, 1H), 6.92-6.89 (m, 1H), 4.82-4.79 (m, 1H), 4.36-4.32 | |
| | (m, 1H), 3.95(s, 3H), 3.89-3.82 (m, 1H), 3.53-3.51 (m, 1H), 2.76 (s, 3H), 2.46 | |
| | (s, 3H), 2.64 (s, 6H), 2.48-2.45 (m, 1H), 2.18-2.12 (m, 2H), 1.91-1.82 (m, | |
| | 1H). | |
| 144 | 1H NMR (400 MHz, CDCl3) δ 8.06 (dd, J = 2 Hz, J = 5.2 Hz, 1H), 7.79 (dd, | 486.3 |
| | J = 1.6 Hz, J = 7.6 Hz, 1H), 6.92-6.89 (m, 1H), 4.82-4.79 (m, 1H), 4.36-4.32 | |
| | (m, 1H), 3.95(s, 3H), 3.89-3.82 (m, 1H), 3.53-3.51 (m, 1H), 2.79 (s, 3H), | |
| | 2.76 (s, 3H), 2.64 (s, 6H), 2.48-2.45 (m, 1H), 2.18-2.12 (m, 2H), 1.91-1.82 | |
| | (m, 1H). | |
| 145 | 1H NMR (400 MHz, CDCl3) δ 7.56 (dd, J = 17.8, 10.0 Hz, 1H), 7.10 (t, J = | 485.7 |
| | 8.7 Hz, 1H), 6.61 (d, J = 8.2 Hz, 1H), 4.56 (d, J = 10.1 Hz, 1H), 4.41-4.29 | |
| | (m, 1H), 4.01-3.76 (m, 4H), 3.58-3.44 (m, 1H), 2.79 (dd, J = 14.3, 8.6 Hz, | |
| | 6H), 2.68-2.55 (m, 7H), 2.18 (dt, J = 26.4, 9.5 Hz, 2H), 2.00 (dd, J = 24.8, | |
| | 12.1 Hz, 1H). | |
| 146 | 1H NMR (400 MHz, CDCl3) δ 7.60-7.53 (m, 1H), 7.09 (d, J = 7.3 Hz, 1H), | 485.7 |
| | 6.61 (d, J = 8.2 Hz, 1H), 4.56 (dd, J = 11.4, 2.0 Hz, 1H), 4.35 (dt, J = 17.3, 8.5 | |
| | Hz, 1H), 3.95-3.77 (m, 4H), 3.53 (tt, J = 11.8, 3.9 Hz, 1H), 2.78 (d, J = 13.2 | |
| | Hz, 6H), 2.62 (d, J = 16.0 Hz, 7H), 2.23-2.08 (m, 2H), 2.00 (dd, J = 25.0, | |
| | 11.9 Hz, 1H). | |
| 147 | 1H NMR (400 MHz, CDCl3) δ 7.75 (dd, J = 15.7, 7.1 Hz, 1H), 7.53 (d, J = | 477.1 |
| | 6.8 Hz, 2H), 7.12-7.07 (m, 1H), 7.00 (td, J = 9.5, 2.4 Hz, 1H), 4.72 (dd, J = | |
| | 16.4, 8.0 Hz, 1H), 4.58 (dd, J = 11.4, 2.0 Hz, 1H), 4.31-4.24 (m, 1H), | |
| | 3.87-3.78 (m, 1H), 3.60-3.51 (m, 1H), 2.83 (d, J = 10.1 Hz, 3H), 2.73 (s, 3H), | |
| | 2.56-2.43 (m, 5H), 2.27-2.16 (m, 3H), 1.89-1.80 (m, 2H). | |
| 148 | 1H NMR (400 MHz, CDCl3) δ 7.75 (dd, J = 15.7, 7.1 Hz, 1H), 7.53 (d, J = | 477.1 |
| | 6.7 Hz, 2H), 7.14-7.06 (m, 1H), 7.00 (td, J = 9.5, 2.4 Hz, 1H), 4.72 (dd, J = | |
| | 16.6, 8.4 Hz, 1H), 4.58 (dd, J = 11.4, 1.9 Hz, 1H), 4.30-4.23 (m, 1H), | |
| | 3.88-3.76 (m, 1H), 3.61-3.47 (m, 1H), 2.84 (d, J = 6.6 Hz, 3H), 2.73 (s, 3H), | |
| | 2.58-2.39 (m, 5H), 2.29-2.12 (m, 3H), 1.91-1.76 (m, 2H). | |
| 149 | 1H NMR (400 MHz, CDCl3) δ 7.80-7.72 (m, 1H), 7.55 (d, J = 16.5 Hz, 2H), | 469.2 |
| | 7.13-7.05 (m, 1H), 7.00 (td, J = 9.4, 2.4 Hz, 1H), 4.80 (t, J = 4.8 Hz, 1H), 4.68 | |
| | (t, J = 4.8 Hz, 1H), 4.59 (dd, J = 11.4, 2.0 Hz, 1H), 4.42 (t, J = 4.8 Hz, 1H), | |
| | 4.35 (t, J = 4.8 Hz, 1H), 4.28 (dt, J = 6.0, 3.3 Hz, 1H), 3.86-3.77 (m, 1H), 3.55 | |
| | (ddd, J = 15.7, 11.8, 3.6 Hz, 1H), 2.85 (d, J = 3.5 Hz, 3H), 2.74 (d, J = 4.2 Hz, | |
| | 3H), 2.46 (d, J = 13.3 Hz, 1H), 2.28-2.14 (m, 3H). | |
| 150 | 1H NMR (400 MHz, CDCl3) δ 7.80-7.71 (m, 1H), 7.57 (s, 1H), 7.53 (d, J = | 469.2 |
| | 3.3 Hz, 1H), 7.09 (ddd, J = 11.2, 5.9, 2.9 Hz, 1H), 7.00 (td, J = 9.4, 2.4 Hz, | |
| | 1H), 4.80 (t, J = 4.8 Hz, 1H), 4.68 (t, J = 4.8 Hz, 1H), 4.59 (dd, J = 11.4, 1.9 | |
| | Hz, 1H), 4.42 (t, J = 4.8 Hz, 1H), 4.35 (t, J = 4.8 Hz, 1H), 4.31-4.24 (m, 1H), | |
| | 3.87-3.77 (m, 1H), 3.54 (td, J = 11.9, 6.0 Hz, 1H), 2.85 (s, 3H), 2.73 (s, 3H), | |
| | 2.46 (d, J = 13.2 Hz, 1H), 2.28-2.13 (m, 3H). | |
| 151 | 1H NMR (DMSO-d6, 400 MHz): δH 8.95 (1H, s), 7.71 (1H, s), 7.37 (1H, s), | 431.3 |
| | 4.53 (1H, d, J = 11.2 Hz), 3.76 (1H, s), 3.64 (1H, t, J = 5.8 Hz), 2.74 (3H, s), | |
| | 2.26 (7H, s), 2.17 (2H, d, J = 13.3 Hz), 2.04 (1H, d, J = 13.4 Hz), 1.85-1.76 | |
| | (1H, m), 1.53 (1H, q, J = 12.1 Hz), 1.27 (3H, s), 1.18 (4H, d, J = 6.1 Hz), 0.99 | |
| | (3H, s), 0.90 (2H, d, J = 7.1 Hz) | |
| 152 | 1H NMR (400 MHz, CDCl3) δ 7.67-7.57 (m, 1H), 7.49 (t, J = 7.9 Hz, 2H), | 481.0 |
| | 7.16-7.00 (m, 1H), 4.56 (dd, J = 11.4, 1.9 Hz, 1H), 4.27 (dd, J = 8.2, 2.7 Hz, | |
| | 1H), 3.87-3.70 (m, 1H), 3.64-3.42 (m, 2H), 2.85 (s, 3H), 2.74 (s, 3H), 2.41 | |
| | (t, J = 19.9 Hz, 1H), 2.18 (ddd, J = 27.7, 13.5, 10.5 Hz, 3H), 1.14-1.02 (m, | |
| | 2H), 1.00-0.85 (m, 2H). | |
| 153 | 1H NMR (400 MHz, CDCl3) δ 7.67-7.54 (m, 1H), 7.50 (d, J = 2.7 Hz, 2H), | 481.0 |
| | 7.12 (td, J = 9.5, 6.3 Hz, 1H), 4.56 (dd, J = 11.4, 1.9 Hz, 1H), 4.27 (dd, J = | |
| | 8.1, 2.8 Hz, 1H), 3.89-3.69 (m, 1H), 3.62-3.40 (m, 2H), 2.85 (s, 3H), 2.74 | |
| | (s, 3H), 2.43 (d, J = 13.2 Hz, 1H), 2.26-2.05 (m, 3H), 1.12-1.04 (m, 2H), | |
| | 1.00-0.91 (m, 2H). | |
| 154 | 1HN MR: (400 MHz, CDCl3) δ 7.76 (dd, J = 15.3, 7.6 Hz, 1H), 7.58 (s, 1H), | 464.1 |
| | 7.11-7.07 (m, 1H), 7.03-6.98 (m, 1H), 4.76 (dd, J = 11.6, 2.1 Hz, 1H), 4.32 | |
| | (dt, J = 5.9, 3.3 Hz, 1H), 3.98-3.92 (m, 1H), 3.87-3.82 (m, 1H), 3.62-3.54 (m, | |
| | 1H), 2.84 (s, 3H), 2.73 (s, 3H), 2.57-2.49 (m, 1H), 2.38-2.29 (m, 1H), | |
| | 2.27-2.20 (m, 2H), 1.37-1.29 (m, 2H), 1.10-1.02 (m, 2H). | |
| 155 | 1H NMR: (400 MHz, CDCl3) δ 7.76 (dd, J = 15.3, 7.6 Hz, 1H), 7.58 (s, 1H), | 464.1 |
| | 7.11-7.07 (m, 1H), 7.03-6.98 (m, 1H), 4.76 (dd, J = 11.6, 2.1 Hz, 1H), 4.32 | |
| | (dt, J = 5.9, 3.3 Hz, 1H), 3.98-3.92 (m, 1H), 3.87-3.82 (m, 1H), 3.62-3.54 (m, | |
| | 1H), 2.84 (s, 3H), 2.73 (s, 3H), 2.57-2.49 (m, 1H), 2.38-2.29 (m, 1H), | |
| | 2.27-2.20 (m, 2H), 1.37-1.29 (m, 2H), 1.10-1.02 (m, 2H). | |
| 156 | 1H NMR (400 MHz, CD2Cl2) δ 7.75 (dd, J = 15.1, 8.0 Hz, 1H), 7.42 (s, | 440.2 |
| | 1H), 7.39 (s, 1H), 7.14-7.07 (m, 1H), 7.05-6.98 (m, 1H), 4.56 (dd, J = | |
| | 11.2, 0.9 Hz, 1H), 4.22 (ddd, J = 13.3, 4.1, 2.2 Hz, 1H), 3.84-3.75 (m, 1H), | |
| | 3.55-3.45 (m, 1H), 2.81 (s, 3H), 2.70 (s, 3H), 2.44-2.37 (m, 1H), | |
| | 2.16-2.04 (m, 3H). 19F NMR (376 MHz, CD2Cl2) δ-107.15 (s), -107.49 (s). | |
| 157 | 1H NMR (400 MHz, CDCl3) δ 7.61 (ddd, J = 10.0, 8.8, 6.2 Hz, 1H), 7.51 (d, | 495.4 |
| | J = 6.2 Hz, 2H), 7.18-7.06 (m, 1H), 4.61 (dd, J = 11.4, 1.9 Hz, 1H), 3.85 | |
| | (ddd, J = 11.1, 6.2, 1.9 Hz, 1H), 3.55 (dt, J = 10.7, 3.6 Hz, 2H), 2.85 (s, 3H), | |
| | 2.74 (s, 3H), 2.46-2.34 (m, 1H), 2.27-2.19 (m, 1H), 2.13 (dd, J = 24.8, | |
| | 12.0 Hz, 1H), 1.85 (dd, J = 24.2, 12.3 Hz, 1H), 1.33 (d, J = 6.2 Hz, 3H), 1.09 | |
| | (dd, J = 3.8, 2.6 Hz, 2H), 0.98 (dd, J = 7.2, 2.3 Hz, 2H). | |
| 158 | 1H NMR (400 MHz, CDCl3) δ 7.66-7.58 (m, 1H), 7.47 (d, J = 3.0 Hz, 2H), | 495.0 |
| | 7.13 (td, J = 9.5, 6.4 Hz, 1H), 4.74 (d, J = 10.0 Hz, 1H), 3.99 (dd, J = 9.6, 6.1 | |
| | Hz, 1H), 3.81 (s, 1H), 3.59-3.48 (m, 1H), 2.86 (s, 3H), 2.83 (d, J = 3.4 Hz, | |
| | 1H), 2.75 (s, 3H), 2.70-2.61 (m, 1H), 2.22-2.13 (m, 1H), 1.93-1.78 (m, | |
| | 1H), 1.26 (d, J = 6.1 Hz, 3H), 1.10-1.05 (m, 2H), 0.98 (dt, J = 7.2, 3.5 Hz, | |
| | 2H). | |
| 159 | 1H NMR (400 MHz, CDCl3) δ 8.54 (d, J = 8.1 Hz, 2H), 7.82 (d, J = 8.3 Hz, | 509.2 |
| | 2H), 7.52 (d, J = 5.0 Hz, 2H), 4.63 (dd, J = 11.5, 1.9 Hz, 1H), 3.87 (ddd, J = | |
| | 11.1, 6.2, 1.9 Hz, 1H), 3.55 (dd, J = 10.2, 6.6 Hz, 2H), 2.86 (s, 3H), 2.80 (s, | |
| | 3H), 2.49-2.38 (m, 1H), 2.33-2.08 (m, 2H), 1.92-1.82 (m, 1H), 1.34 (d, J = | |
| | 6.2 Hz, 3H), 1.09 (dt, J = 7.2, 3.6 Hz, 2H), 0.99 (dt, J = 7.2, 3.8 Hz, 2H). | |
| 160 | 1H NMR (500 MHz, CDCl3) δ 8.06 (d, J = 4.9 Hz, 1H), 8.06 (d, J = 4.9 Hz, | 486.3 |
| | 1H), 8.35-7.70 (m, 3H), 7.77 (dd, J = 29.0, 7.4 Hz, 2H), 7.08 (s, 1H), 6.99 | |
| | (s, 1H), 6.90 (t, J = 6.1 Hz, 1H), 4.83 (d, J = 11.1 Hz, 1H), 4.35 (d, J = 9.7 Hz, | |
| | 1H), 3.95 (s, 3H), 3.85 (s, 1H), 3.61 (d, J = 11.5 Hz, 1H), 2.85 (d, J = 21.4 Hz, | |
| | 3H), 2.74 (d, J = 20.4 Hz, 3H), 2.53 (d, J = 12.8 Hz, 1H), 2.22 (s, 2H), 1.94 | |
| | (d, J = 12.3 Hz, 1H). | |
| 161 | 1H NMR (500 MHz, CDCl3) δ 8.06 (dd, J = 5.0, 1.7 Hz, 1H), 7.80 (dd, J = | 486.3 |
| | 7.3, 1.5 Hz, 1H), 7.74 (d, J = 6.8 Hz, 1H), 7.08 (d, J = 2.0 Hz, 1H), 6.99 | |
| | (d, J = 2.0 Hz, 1H), 6.90 (dd, J = 7.3, 5.1 Hz, 1H), 4.83 (d, J = 10.0 Hz, 1H), | |
| | 4.35 (dd, J = 10.4, 3.0 Hz, 1H), 3.95 (s, 3H), 3.85 (s, 1H), 3.63 (m, 1H), 2.82 | |
| | (s, 3H), 2.72 (s, 3H), 2.55 (t, J = 17.5 Hz, 1H), 2.21 (dd, J = 9.9, 3.9 Hz, 2H), | |
| | 2.08-1.85 (m, 1H). | |
| 162 | 1H NMR (400 MHz, CDCl3) δ 8.1 (d, J = 2.4 Hz, 1H), 7.77-7.68 (m, 2H), | 462.1 |
| | 7.11-6.97 (m, 2H), 6.73 (d, J = 8.8 Hz, 1H), 4.53 (dd, J = 1.6 Hz, J = 11.6 Hz, | |
| | 1H), 4.35-4.32 (m, 1H), 3.92(s, 3H), 3.87-3.34 (m, 1H), 3.59 (m, 1H), 2.84 | |
| | (s, 3H), 2.73 (s, 3H), 2.37 (dd, J = 2.4 Hz, J = 12.8 Hz, 1H), 2.24-2.14 (m, | |
| | 3H). | |
| 163 | 1H NMR (400 MHz, CDCl3) δ 8.1 (d, J = 2.4 Hz, 1H), 7.77-7.68 (m, 2H), | 462.1 |
| | 7.11-6.98 (m, 2H), 6.73 (d, J = 8.4 Hz, 1H), 4.53 (dd, J = 1.6 Hz, J = 11.2 Hz, | |
| | 1H), 4.35-4.32 (m, 1H), 3.92(s, 3H), 3.87-3.34 (m, 1H), 3.59 (m, 1H), 2.84 | |
| | (s, 3H), 2.75 (s, 3H), 2.37 (dd, J = 2.0 Hz, J = 13.6 Hz, 1H), 2.23-2.11 (m, | |
| | 3H). | |
| 164 | ¹H NMR (400 MHz, CDCl3) δ 7.75 (dd, J = 14.8, 8.0 Hz, 1H), 7.60-7.52 (m, | 464.4 |
| | 1H), 7.08 (dd, J = 10.2, 4.9 Hz, 2H), 7.04-6.94 (m, 1H), 6.61 (d, J = 8.2 Hz, | |
| | 1H), 4.63-4.49 (m, 1H), 4.44-4.30 (m, 1H), 3.90 (s, 3H), 3.85 (dd, J = | |
| | 11.8, 2.9 Hz, 1H), 3.69-3.55 (m, 1H), 2.83 (s, 3H), 2.72 (s, 3H), 2.68 (d, J = | |
| | 11.4 Hz, 1H), 2.33-2.16 (m, 2H), 2.07 (dd, J = 25.0, 12.0 Hz, 1H) | |
| 165 | ¹H NMR (400 MHz, CDCl3) δ 7.75 (dd, J = 14.7, 8.1 Hz, 1H), 7.56 (dd, J = | 464.4 |
| | 17.1, 9.3 Hz, 1H), 7.08 (dd, J = 10.5, 4.8 Hz, 2H), 6.99 (td, J = 9.4, 2.4 Hz, | |
| | 1H), 6.61 (d, J = 8.2 Hz, 1H), 4.58 (d, J = 9.4 Hz, 1H), 4.44-4.27 (m, 1H), | |
| | 3.90 (s, 3H), 3.85 (dd, J = 11.8, 2.8 Hz, 1H), 3.67-3.54 (m, 1H), 2.83 (s, | |
| | 3H), 2.72 (s, 3H), 2.68 (d, J = 11.2 Hz, 1H), 2.34-2.14 (m, 2H), 2.07 (dd, | |
| | J = 24.9, 12.0 Hz, 1H) | |
| 166 | ¹H NMR (400 MHz, CDCl3) δ 7.75 (dd, J = 15.0, 7.8 Hz, 1H), 7.08 (t, J = 8.3 | 463.2 |
| | Hz, 1H), 7.00 (t, J = 9.5 Hz, 1H), 5.93 (s, 1H), 4.66 (d, J = 11.3 Hz, 1H), 4.30 | |
| | (d, J = 11.4 Hz, 1H), 3.84 (d, J = 13.5 Hz, 1H), 3.55 (s, 1H), 2.84 (s, 3H), | |
| | 2.73 (s, 3H), 2.50 (d, J = 13.7 Hz, 1H), 2.24 (s, 1H), 2.01 (d, J = 5.6 Hz, 1H), | |
| | 1.87 (d, J = 3.9 Hz, 1H), 1.64 (s, 1H), 0.89 (d, J = 7.0 Hz, 2H), 0.70 (d, J = | |
| | 5.2 Hz, 2H) | |
| 167 | ¹H NMR (400 MHz, CDCl3) δ 7.79-7.64 (m, 3H), 7.11-7.07 (t, J = 8 Hz, 1H), | 423 |
| | 7.03-6.98 (t, J = 8.8 Hz, 1H), 4.66-4.63 (d, J = 11.2 Hz, 1H), 4.31-4.27 (d, J = | |
| | 11.6 Hz, 1H), 3.95-3.80 (m, 1H), 3.60-3.53 (m, 1H), 2.848 (s, 3H), 2.734 (s, | |
| | 3H), 2.65 (d, J = 13.2 Hz, 1H), 2.29-2.20 (m, 3H). | |
| 168 | ¹H NMR (400 MHz, CDCl3) δ 7.8-7.67 (m, 3H), 7.12-6.98 (m, 2H), 4.87-4.68 | 437 |
| | (m, 1H), 4.04-3.56 (m, 2H), 2.85 (d, J = 4.8 Hz, 3H), 2.74 (d, J = 6 Hz, 3H), | |
| | 2.44 (d, J = 13.2 Hz, 1H), 2.27-2.11 (m, 2H), 1.93-1.84 (m, 1H), 1.35-1.23 | |
| | (dd, J = 6 Hz, J = 30 Hz, 3H). | |
| 169 | ¹H NMR (400 MHz, CDCl3) δ 7.80-7.70 (m, 1H), 7.12-7.04 (m, 1H), | 464.3 |
| | 7.03-6.95 (m, 1H), 4.82-4.72 (m, 1H), 4.38-4.23 (m, 1H), 3.90-3.78 (m, 1H), | |
| | 3.63-3.50 (m, 1H), 2.83 (s, 3H), 2.72 (s, 3H), 2.71-2.64 (m, 1H), 2.26-2.17 | |
| | (m, 3H), 2.04-1.98 (m, 1H), 1.03-0.93 (m, 4H). | |
| 170 | ¹H NMR (DMSO-d6, 400 MHz): δH 7.78-7.84 (1H, m), 7.73 (1H, s), 7.48 | 465.2 |
| | (1H, t, J = 10.0 Hz), 7.39 (1H, s), 7.30-7.33 (1H, m), 4.51 (1H, d, J = 11.2 | |
| | Hz), 3.65 (1H, s), 3.43-3.47 (1H, m), 2.78 (3H, s), 2.66 (3H, s), 2.30 (1H, d, | |
| | J = 13.6 Hz), 2.05 (1H, d, J = 13.5 Hz), 1.93 (2H, t, J = 12.5 Hz), 0.99 (2H, | |
| | s), 0.91 (3H, s). | |
| 171 | 1H NMR (DMSO-d6, 400 MHz): δH 7.78-7.84 (1H, m), 7.73 (1H, s), 7.48 | 465.2 |
| | (1H, t, J = 9.7 Hz), 7.38 (1H, s), 7.32 (1H, t, J = 8.6 Hz), 4.51 (1H, d, J = | |
| | 11.2 Hz), 3.65 (1H, s), 3.43-3.47 (1H, m), 2.78 (3H, s), 2.66 (3H, s), 2.30 (1H, | |
| | d, J = 13.4 Hz), 2.05 (1H, d, J = 13.4 Hz), 1.93 (2H, t, J = 12.4 Hz), 0.99 | |
| | (2H, s), 0.91 (2H, s). | |
| 172 | 1H NMR (400 MHz, CDCl3) a 8.36 (d, J = 7.9 Hz, 2H), 7.51 (s, 1H), 7.41 | 424.0 |
| | (s, 1H), 7.37 (d, J = 7.9 Hz, 2H), 4.58 (d, J = 11.3 Hz, 1H), 4.32-4.23 (m, | |
| | 1H), 3.87-3.79 (m, 1H), 3.57-3.48 (m, 1H), 2.47 (s, 3H), 2.44 (s, 1H), | |
| | 2.29-2.15 (m, 3H). | |
| 173 | 1H NMR (400 MHz, CDCl3) δ 8.37 (d, J = 7.6 Hz, 2H), 7.51 (s, 1H), 7.41 (s, | 424.05 |
| | 1H), 7.37 (d, J = 7.6 Hz, 2H), 4.58 (d, J = 11.2 Hz, 1H), 4.32-4.21 (m, 1H), | |
| | 3.88-3.77 (m, 1H), 3.58-3.48 (m, 1H), 2.47 (s, 3H), 2.44 (s, 1H), | |
| | 2.28-2.16 (m, 3H). | |
| 174 | 1H NMR (400 MHz, CDCl3) δ 8.37 (d, J = 7.8 Hz, 2H), 7.51 (s, 1H), 7.41 | 427.1 |
| | (s, 1H), 7.37 (d, J = 7.7 Hz, 2H), 4.58 (d, J = 11.5 Hz, 1H), 4.31-4.22 (m, | |
| | 1H), 3.88-3.77 (m, 1H), 3.58-3.46 (m, 1H), 2.50-2.41 (m, 1H), | |
| | 2.29-2.15 (m, 3H). | |
| 175 | 1H NMR (400 MHz, CDCl3) δ 8.37 (d, J = 8.0 Hz, 2H), 7.51 (s, 1H), 7.41 (s, | 427.1 |
| | 1H), 7.37 (d, J = 7.9 Hz, 2H), 4.58 (d, J = 11.2 Hz, 1H), 4.27 (d, J = 11.5 Hz, | |
| | 1H), 3.87-3.78 (m, 1H), 3.58-3.48 (m, 1H), 2.46 (d, J = 13.8 Hz, 1H), | |
| | 2.29-2.17 (m, 3H). | |
| 176 | 1H NMR (400 MHz, CDCl3) δ 8.45 (d, J = 5.1 Hz, 1H), 7.45 (dd, J = 7.8, | 492.9 |
| | 5.3 Hz, 1H), 7.37 (dd, J = 8.0, 6.7 Hz, 1H), 7.22 (s, 1H), 7.12 (d, J = 4.9 | |
| | Hz, 1H), 4.53 (d, J = 11.4 Hz, 1H), 4.40-4.33 (m, 1H), 3.88-3.78 (m, 1H), | |
| | 3.66-3.54 (m, 1H), 2.47-2.41 (m, 1H), 2.25-2.19 (m, 2H), 2.10-1.98 (m, | |
| | 1H). Note: H/D isotopic peaks are seen at 2.71 and 2.82 ppm. 19F NMR (376 | |
| | MHz, CDCl3) δ-131.93 (s), -137.57 (s). | |
| 177 | 1H NMR (400 MHz, CDCl3) δ 8.45 (d, J = 5.4 Hz, 1H), 7.46 (dd, J = 9.4, 5.8 | 492.9 |
| | Hz, 1H), 7.37 (dd, J = 7.7, 6.0 Hz, 1H), 7.22 (s, 1H), 7.12 (d, J = 4.6 Hz, 1H), | |
| | 4.53 (d, J = 11.1 Hz, 1H), 4.40-4.33 (m, 1H), 3.88-3.78 (m, 1H), 3.65-3.55 | |
| | (m, 1H), 2.48-2.40 (m, 1H), 2.26-2.18 (m, 2H), 2.11-1.98 (m, 1H). | |
| | Note: H/D isotopic peaks are seen at 2.71 and 2.83 ppm. 19F NMR (376 | |
| | MHz, CDCl3) δ-131.84 (s), -137.59 (s). | |
| 178 | | |
| 179 | 1H NMR (400 MHz, CDCl3) δ 7.72-7.66 (m, 1H), 7.50 (d, J = 1.3 Hz, | 480.9 |
| | 2H), 7.35 (dd, J = 8.3, 1.8 Hz, 1H), 7.29 (dd, J = 9.6, 1.8 Hz, 1H), 4.55 (dd, | |
| | J = 11.4, 1.7 Hz, 1H), 4.26 (dt, J = 5.7, 3.2 Hz, 1H), 3.86-3.75 (m, 1H), | |
| | 3.53 (ddd, J = 12.2, 6.6, 2.7 Hz, 1H), 2.84 (s, 3H), 2.73 (s, 3H), 2.47-2.40 | |
| | (m, 1H), 2.28-2.15 (m, 3H), 1.08 (t, J = 6.2 Hz, 2H), 0.97 (t, J = 6.1 Hz, | |
| | 2H). 19F NMR (376 MHz, CDCl3) δ-108.28 (s). | |
| 194 | 1H NMR (400 MHz, Chloroform-d) δ ppm 8.45 (1H, d, J = 5.2 Hz), 8.01 (1H, | 484.2 |
| | dd, J = 8.5, 4.0 Hz), 7.65-7.59 (1H, m), 7.45 (1H, m), 7.27-7.26 (1H, m), | |
| | 7.23-7.02 (3H, m), 4.55 (1H, d, J = 11.5 Hz), 4.37 (1H, d, J = 11.5 Hz), | |
| | 3.85-3.78 (1H, m), 3.62-3.54 (1H, m), 2.85 (3H, s), 2.59 (3H, s), 2.46-2.42 | |
| | (1H, m), 2.27-2.18 (2H, m), 2.12-1.98 (1H, m). 19F NMR (376 MHz, | |
| | Chloroform-d) δF-105.6, -108.9. | |
| 195 | 1H NMR (400 MHz, Methylene Chloride-d2) δ ppm 9.70 (s, 1H), 8.91 (d, J = | 484.10 |
| | 8.4 Hz, 1H), 7.88 (d, J = 8.2 Hz, 1H), 7.33 (d, J = 4.5 Hz, 1H), 6.65 (s, 1H), | |
| | 6.34 (d, J = 7.2 Hz, 1H), 5.11-4.99 (m, 1H), 4.90 (d, J = 13.5 Hz, 1H), 4.42 | |
| | (d, J = 8.7 Hz, 1H), 4.20 (d, J = 12.1 Hz, 1H), 3.82 (t, J = 11.2 Hz, 1H), | |
| | 3.38-3.26 (m, 1H), 3.06 (dd, J = 13.7, 9.8 Hz, 1H), 2.72 (s, 3H), 2.67 (s, 3H). | |
| | Note: (1) One exchangeable proton is not visible. 19F NMR (376 MHz, | |
| | Methylene Chloride-d2) δ ppm-68.40 | |
| 196 | 1H NMR (400 MHz, Methylene Chloride-d2) δ ppm 9.70 (s, 1H), 8.91 (d, J = | 484.15 |
| | 8.4 Hz, 1H), 7.88 (d, J = 8.2 Hz, 1H), 7.33 (d, J = 4.5 Hz, 1H), 6.65 (s, 1H), | |
| | 6.34 (d, J = 7.2 Hz, 1H), 5.11-4.99 (m, 1H), 4.90 (d, J = 13.5 Hz, 1H), 4.42 | |
| | (d, J = 8.7 Hz, 1H), 4.20 (d, J = 12.1 Hz, 1H), 3.82 (t, J = 11.2 Hz, 1H), | |
| | 3.38-3.26 (m, 1H), 3.06 (dd, J = 13.7, 9.8 Hz, 1H), 2.72 (s, 3H), 2.67 (s, 3H). | |
| | Note: (1) One exchangeable proton is not visible. 19F NMR (376 MHz, | |
| | Methylene Chloride-d2) δ ppm-68.40 | |
| 197 | 1H NMR (400 MHz, Chloroform-d) δ ppm 8.47 (1H, m), 7.53 (1H, s), 7.23 | 473.8 |
| | (1H, s), 7.13 (1H, m), 4.54 (1H, d, J = 11.3 Hz), 4.36 (1H, d, J = 11.4 Hz), | |
| | 3.86-3.80 (1H, m), 3.29 (1H, m), 2.73 (3H, s), 2.72 (3H, s), 2.60 (6H, s), 2.57 | |
| | (3H, s), 2.30 (1H, d, J = 13.5 Hz), 2.22-2.15 (1H, m), 2.05 (2H, m), 1.82-1.73 | |
| | (1H, m). | |
| 198 | 1H NMR (400 MHz, Methylene Chloride-d2) δ ppm 11.33 (s, 1H), 7.34 | 473.20 |
| | (d, J = 6.8 Hz, 1H), 6.61 (s, 1H), 6.34 (d, J = 6.8 Hz, 1H), 4.99 (d, J = 13.0 | |
| | Hz, 1H), 4.82 (d, J = 14.0 Hz, 1H), 4.41 (d, J = 8.3 Hz, 1H), 4.15 (d, J = 11.4 | |
| | Hz, 1H), 3.82-3.72 (m, 1H), 3.30-3.20 (m, 1H), 3.00 (dd, J = 13.4, 10.5 | |
| | Hz, 1H), 2.65 (s, 3H), 2.62 (s, 3H), 2.60 (s, 6H). 19F NMR (376 MHz, | |
| | Methylene Chloride-d2) δ ppm-73.46 (s). | |
| 199 | 1H NMR (400 MHz, Methylene Chloride-d2) δ ppm 8.48 (d, J = 5.0 Hz, 1H), | 473.20 |
| | 8.42 (s, 1H), 7.26 (s, 1H), 7.17 (d, J = 5.2 Hz, 1H), 6.82 (s, 1H), 4.61 (dd, J = | |
| | 10.4, 2.7 Hz, 1H), 4.50 (d, J = 13.1 Hz, 1H), 4.27 (d, J = 12.7 Hz, 1H), | |
| | 4.23-4.16 (m, 1H), 3.92-3.84 (m, 1H), 3.20-3.10 (m, 1H), 2.87 (dd, J = 12.9, | |
| | 10.5 Hz, 1H), 2.64 (s, 3H), 2.57 (s, 6H), 2.56 (s, 3H). 19F NMR (376 MHz, | |
| | Methylene Chloride-d2) δ ppm-73.39 (s). | |
| 200 | ¹H NMR (400 MHz, CDCl3) δ 7.775-7.718 (m, 1H), 7.420-7.392 (m, 2H), | 464.2 |
| | 7.116-7.070 (m, 1H), 7.033-6.980 (m, 1H), 6.582-6.557 (m, 1H), 4.352-4.279 | |
| | (m, 1H), 3.832-3.766 (m, 1H), 3.575-3.520 (m, 4H), 2.845 (s, 3H), 2.732 (s, | |
| | 3H), 2.37 (d, J = 13.2 Hz, 1H), 2.216-2.051 (m, 3H). | |
| 201 | ¹H NMR (400 MHz, CDCl3) δ 7.775-7.718 (m, 1H), 7.420-7.392 (m, 2H), | 464.2 |
| | 7.116-7.070 (m, 1H), 7.033-6.980 (m, 1H), 6.582-6.557 (m, 1H), 4.352-4.279 | |
| | (m, 1H), 3.832-3.766 (m, 1H), 3.575-3.520 (m, 4H), 2.845 (s, 3H), 2.732 (s, | |
| | 3H), 2.37 (d, J = 13.2 Hz, 1H), 2.216-2.051 (m, 3H). | |
| 202 | ¹H NMR (400 MHz, CDCl3) δ 7.76 (d, J = 6.6 Hz, 1H), 7.31 (s, 1H), 7.10 (s, | 464.7 |
| | 1H), 7.00 (s, 1H), 6.56 (d, J = 8.3 Hz, 1H), 6.43 (d, J = 6.5 Hz, 1H), 4.54 (d, | |
| | J = 9.7 Hz, 1H), 4.38-4.32 (m, 1H), 3.88-3.78 (m, 1H), 3.65 (s, 3H), 3.59-3.55 | |
| | (m, 1H), 2.85 (s, 3H), 2.74 (s, 3H), 2.26-2.18 (m, 2H), 2.05-1.97 (m, 2H). | |
| 203 | ¹H NMR (400 MHz, CDCl₃) δ 7.77-7.71 (m, 1H), 7.56 (d, J = 5.6 Hz, 1H), | 464.1 |
| | 7.29 (dd, J = 1.6 Hz, J = 6.8 Hz, 1H), 7.1-7.05 (m, 1H), 7.0-6.95 (m, 1H), | |
| | 6.20 (t, 1H), 4.83 (d, J = 10.4 Hz, 1H), 4.34-4.30 (m, 1H), 3.90-3.83 (m, | |
| | 1H), 3.66-3.64 (m, 1H), 3.54 (s, 3H), 2.82. (s, 3H), 2.71 (s, 3H), 2.65 (d, J = | |
| | 13.6 Hz, 1H), 2.19-2.14 (m, 2H), 1.95-1.88 (m, 1H). | |
| 204 | ¹H NMR (400 MHz, CDCl₃) δ 7.77-7.71 (m, 1H), 7.56 (d, J = 5.6 Hz, 1H), | 464.1 |
| | 7.29 (dd, J = 1.6 Hz, J = 6.8 Hz, 1H), 7.1-7.05 (m, 1H), 7.0-6.95 (m, 1H), | |
| | 6.20 (t, 1H), 4.83 (d, J = 10.4 Hz, 1H), 4.34-4.30 (m, 1H), 3.90-3.83 (m, | |
| | 1H), 3.66-3.64 (m, 1H), 3.54 (s, 3H), 2.82. (s, 3H), 2.71 (s, 3H), 2.65 (d, J = | |
| | 13.6 Hz, 1H), 2.19-2.14 (m, 2H), 1.95-1.88 (m, 1H). | |
| 208 | ¹H NMR (400 MHz, CDCl3) δ 8.44 (s, 1H), 7.65-7.59 (m, 1H), 7.54 (s, 2H), | 463.1 |
| | 7.03 (td, J = 8.2, 2.4 Hz, 1H), 6.98 (s, 1H), 6.95 (dd, J = 9.5, 2.4 Hz, 1H), 4.68 | |
| | (dd, J = 10.9, 2.6 Hz, 1H), 4.48 (d, J = 12.6 Hz, 1H), 4.31 (d, J = 12.7 Hz, | |
| | 1H), 3.93-3.86 (m, 1H), 3.57 (td, J = 7.2, 3.6 Hz, 1H), 3.03-2.96 (m, 1H), | |
| | 2.81-2.74 (m, 1H), 2.73 (s, 3H), 1.34 (d, J = 6.2 Hz, 3H), 1.12 (dd, J = 6.9, | |
| | 4.3 Hz, 2H), 1.01 (t, J = 6.0 Hz, 2H). | |
| 209 | 1H NMR (400 MHz, CDCl3) δ 8.43 (s, 1H), 7.62 (td, J = 8.2, 6.6 Hz, 1H), | 463.1 |
| | 7.53 (d, J = 7.1 Hz, 2H), 7.03 (td, J = 8.3, 2.1 Hz, 1H), 6.98 (dd, J = 9.1, 6.6 | |
| | Hz, 1H), 6.96-6.92 (m, 1H), 4.68 (dd, J = 10.9, 2.7 Hz, 1H), 4.47 (d, J = 12.6 | |
| | Hz, 1H), 4.30 (d, J = 12.7 Hz, 1H), 3.91 (ddd, J = 10.5, 6.3, 2.7 Hz, 1H), 3.58 | |
| | (ddd, J = 11.0, 7.3, 3.7 Hz, 1H), 2.99 (dd, J = 12.8, 11.0 Hz, 1H), 2.77 (dd, J = | |
| | 12.7, 10.7 Hz, 1H), 2.71 (s, 3H), 1.34 (d, J = 6.2 Hz, 3H), 1.15-1.10 (m, 2H), | |
| | 1.01 (t, J = 6.1 Hz, 2H). | |
| 210 | 1H-NMR (400 MHz, DMSO) δ 7.83 (s, 1H), 7.83-7.63 (m, 2H), 7.50-7.35 (m, | 462.1 |
| | 2H), 7.32-7.21 (m, 1H), 4.49 (d, J =9.5 Hz, 1H), 4.17-4.02 (m, 1H), 3.73-3.61 | |
| | (m, 2H), 3.30 (d, J =11.7 Hz, 1H), 2.73 (s, 3H), 2.65 (d, J = 20.7 Hz, 3H), 2.21 | |
| | (d, J = 13.2Hz, 1H), 2.00-1.81 (m, 3H), 1.00-0.83 (m, 4H). | |
| 211 | 1H NMR (400 MHz, CDCl3) δ 7.84-7.76 (s, 1H), 7.75 (s, 1H), 7.59 (d, J = | 512.3 |
| | 7.9 Hz, 1H), 7.52-7.44 (m, 2H), 4.56 (dd, J = 11.2, 1.8 Hz, 1H), 4.27 (dd, J = | |
| | 11.6, 2.7 Hz, 1H), 3.81 (m, J = 11.6, 2.7 Hz, 1H), 3.56 (m, J = 11.1, 7.4, 3.8 | |
| | Hz, 1H), 3.35 (m, J = 15.7, 7.9, 3.7 Hz, 1H), 2.77 (s, 3H), 2.69 (d, J = 4.1 Hz, | |
| | 3H), 2.39 (d, J = 13.0 Hz, 1H), 2.15-1.88 (m, 3H), 1.10 (m, J = 7.1, 4.4 Hz, | |
| | 2H), 1.02-0.95 (m, 2H). | |
| 212 | 1H NMR (DMSO-d6, 400 MHz): δH 7.80 (1H, s), 7.73 (1H, s), 7.64-7.70 | 476.200 |
| | (1H, m), 7.35-7.41 (2H, m), 7.24 (1H, td, J = 8.5, 2.5 Hz), 4.52 (1H, d, J = | |
| | 11.1 Hz), 3.75 (1H, dd, J = 10.8, 6.2 Hz), 3.63 (1H, tt, J = 7.3, 3.9 Hz), 3.35 | |
| | (1H, m), 2.71 (3H, s), 2.60 (3H, s), 2.14 (1H, d, J = 13.0 Hz), 2.00 (1H, d, | |
| | J = 12.9 Hz), 1.85 (1H, q, J = 12.1 Hz), 1.53 (1H, q, J = 12.0 Hz), 1.18 (3H, | |
| | d, J = 6.1 Hz), 0.95-0.98 (2H, m), 0.86-0.93 (2H, m). | |
| 213 | 1H NMR (CHCl3-d, 400 MHz): δH 7.70 (1H, s), 7.62-7.68 (1H, m), 7.49 | 476.200 |
| | (2H, s), 7.05 (1H, t, J = 8.4 Hz), 6.96 (1H, td, J = 9.4, 2.4 Hz), 4.61 (1H, d, | |
| | J = 11.2 Hz), 3.85 (1H, dd, J = 10.9, 6.0 Hz), 3.51-3.56 (1H, m), 3.36 (1H, | |
| | t, J = 12.1 Hz), 2.76 (3H, s), 2.68 (3H, s), 2.35 (1H, d, J = 13.1 Hz), 2.15 | |
| | (1H, d, J = 13.1 Hz), 1.93 (1H, q, J = 12.2 Hz), 1.61-1.70 (1H, m), 1.33 | |
| | (3H, d, J = 6.2 Hz), 1.10 (2H, t, J = 3.5 Hz), 0.95-1.00 (2H, m). | |
| 214 | 1H NMR (DMSO-d6, 400 MHz): δH 8.73 (1H, d, J = 8.5 Hz), 7.71 (1H, s), | 443.6 |
| | 7.53 (1H, d, J = 8.5 Hz), 7.36 (1H, s), 4.48 (1H, d, J = 11.2 Hz), 4.09-4.05 | |
| | (1H, m), 3.66 (2H, d, J = 13.7 Hz), 3.27 (2H, s), 2.69 (3H, s), 2.17 (7H, s), | |
| | 1.96 (1H, s), 1.86 (2H, br s), 1.77 (1H, s), 1.22 (1H, s), 0.98 (2H, s), 0.89 (6H, | |
| | d, J = 6.8 Hz) | |
| 215 | 1H NMR (400 MHz, CD2Cl2) δ 8.01 (dd, J = 8.5, 4.0 Hz, 1H), 7.69-7.61 | 448.2 |
| | (m, 1H), 7.48 (s, 1H), 7.43-7.38 (m, 2H), 7.15 (td, J = 8.2, 2.0 Hz, 1H), | |
| | 7.09-7.02 (m, 1H), 4.53 (dd, J = 11.4, 1.8 Hz, 1H), 4.25-4.16 (m, 1H), 3.79 | |
| | (ddd, J = 14.8, 8.7, 3.2 Hz, 1H), 3.58-3.50 (m, 1H), 3.49-3.42 (m, 1H), | |
| | 2.81 (s, 3H), 2.44-2.35 (m, 1H), 2.17-2.03 (m, 3H), 1.09-1.03 (m, 2H), | |
| | 1.00-0.91 (m, 2H). 19F NMR (376 MHz, CD2Cl2) δ-107.07 (s), -109.74 (s) | |
| 216 | 1H NMR (400 MHz, MeOD) δ 7.95 (d, J = 3.2 Hz, 1H), 7.72-7.66 (m, 2H), | 478.2 |
| | 7.55-7.47 (m, 3H), 4.97 (dd, J = 8.4, 8.0 Hz, 1H), 3.87 (m, 2H), 3.64 (m, | |
| | 2H), 2.79 (s, 3H), 2.70 (m, 1H), 2.49 (s, 3H), 2.42 (m, 1H), 2.32 (m, 1H), 2.16 | |
| | (m, 1H), 1.04 (m, 4H). | |
| 217 | 1H NMR (400 MHz, CD2Cl2) δ 8.24 (d, J = 8.5 Hz, 1H), 7.92-7.84 (m, | 480.2 |
| | 4H), 7.48 (s, 1H), 7.42 (d, J = 8.3 Hz, 2H), 4.54 (dd, J = 11.3, 1.7 Hz, 1H), | |
| | 4.25-4.18 (m, 1H), 3.84-3.74 (m, 1H), 3.58-3.44 (m, 2H), 2.81 (s, 3H), | |
| | 2.45-2.34 (m, 1H), 2.18-2.05 (m, 3H), 1.10-1.03 (m, 2H), 0.99-0.92 (m, | |
| | 2H). 19F NMR (376 MHz, CD2Cl2) δ-63.13 (s) | |
| 218 | 1H NMR (400 MHz, CD2Cl2) δ 7.99 (dd, J = 8.6, 3.8 Hz, 1H), 7.50-7.40 | 482.1 |
| | (m, 4H), 7.40-7.34 (m, 1H), 4.53 (dd, J = 11.5, 1.9 Hz, 1H), 4.25-4.16 (m, | |
| | 1H), 3.82-3.73 (m, 1H), 3.57-3.50 (m, 1H), 3.50-3.43 (m, 1H), 2.82 (s, | |
| | 3H), 2.43-2.35 (m, 1H), 2.17-2.02 (m, 3H), 1.09-1.03 (m, 2H), 0.99-0.91 | |
| | (m, 2H). 19F NMR (376 MHz, CD2Cl2) δ-135.53 (s), -137.81 (s). | |
| 219 | 1H NMR (400 MHz, CD2Cl2) δ 8.00 (dd, J = 8.5, 4.2 Hz, 1H), 7.52-7.46 | 482.200 |
| | (m, 2H), 7.45-7.36 (m, 3H), 4.54 (dd, J = 11.3, 1.8 Hz, 1H), 4.25-4.17 (m, | |
| | 1H), 3.78 (ddd, J = 11.5, 4.0, 3.4 Hz, 1H), 3.54 (ddd, J = 11.4, 7.6, 3.9 Hz, | |
| | 1H), 3.50-3.42 (m, 1H), 2.81 (s, 3H), 2.39 (dd, J = 13.1, 1.8 Hz, 1H), | |
| | 2.18-2.03 (m, 3H), 1.11-1.03 (m, 2H), 0.96 (dt, J = 12.5, 6.2 Hz, 2H). | |
| | 19F NMR (376 MHz, CD2Cl2) δ-117.12 (s), -120.40 (s). | |
| 220 | 1H NMR (DMSO-d6, 400 MHz): δH 8.76 (1H, d, J = 8.5 Hz), 7.71 (1H, s), | 484.3 |
| | 7.56 (1H, d, J = 8.5 Hz), 7.37 (1H, s), 4.53 (1H, d, J = 11.3 Hz), 3.76 (1H, s), | |
| | 3.64 (1H, d, J = 6.0 Hz), 2.71 (3H, s), 2.64 (6H, s), 2.18 (1H, d, J = 13.1 Hz), | |
| | 2.04 (1H, d, J = 13.4 Hz), 1.82 (1H, q, J = 12.2 Hz), 1.54 (1H, q, J = 12.1 Hz), | |
| | 1.22 (1H, s), 1.19 (3H, d, J = 6.1 Hz), 0.99 (2H, s), 0.90 (2H, d, J = 7.1 Hz) | |
| 221 | 1H NMR (CHCl3-d, 400 MHz): δH 8.44 (1H, d, J = 8.5 Hz), 7.49 (2H, s), | 470.3 |
| | 7.41 (1H, d, J = 8.5 Hz), 4.54 (1H, d, J = 11.4 Hz), 4.25 (1H, d, J = 11.5 Hz), | |
| | 3.80 (1H, s), 3.55 (1H, s), 3.42 (1H, br s), 2.83 (3H, s), 2.62 (6H, s), 2.38 (1H, | |
| | d, J = 13.3 Hz), 2.15 (3H, d, J = 18.0 Hz), 1.09 (2H, s), 0.98 (2H, d, J = 7.0 | |
| | Hz) | |
| 222 | 1H NMR: (400 MHz, DMSO-d6) δ 8.17 (s, 1H), 7.79-7.70 (m, 2H), 7.65 (dd, | 478.2 |
| | J = 9.8, 1.9 Hz, 1H), 7.50 (dd, J = 8.3, 1.9 Hz, 1H), 7.38 (s, 1H), 4.50 (d, J = | |
| | 9.6 Hz, 1H), 4.15-4.04 (m, 1H), 3.75-3.60 (m, 2H), 3.47-3.41 (m, 1H), | |
| | 2.58 (s, 3H), 2.52 (s, 3H), 2.32-2.21 (m, 1H), 2.06-1.86 (m, 3H), | |
| | 1.02-0.96 (m, 2H), 0.95-0.88 (m, 2H). | |
| 223 | 1H NMR (400 MHz, CDCl3) δ 7.58-7.45 (m, 4H), 7.33-7.25 (m, 2H), 4.55 | 478.2 |
| | (dd, J = 11.2, 2.0 Hz, 1H), 4.31-4.19 (m, 1H), 3.80 (td, J = 11.9, 2.2 Hz, | |
| | 1H), 3.55 (dq, J = 11.1, 3.8 Hz, 1H), 3.38 (tt, J = 11.9, 3.7 Hz, 1H), 2.81 (s, | |
| | 3H), 2.69 (s, 3H), 2.32 (dd, J = 11.5, 1.6 Hz, 1H), 2.25-2.10 (m, 2H), | |
| | 2.08-1.97 (m, 1H), 1.14-1.03 (m, 2H), 1.01-0.84 (m, 2H). | |
| 224 | 1H NMR (400 MHz, CDCl3) δ 7.61-7.42 (m, 4H), 7.30-7.28 (m, 2H), 5.05 | 478.1 |
| | (t, J = 4.8 Hz, 1H), 4.02-3.80 (m, 2H), 3.57 (tdd, J = 12.1, 7.8, 3.9 Hz, 2H), | |
| | 2.82 (s, 3H), 2.69 (s, 3H), 2.60-2.50 (m, 1H), 2.41-2.19 (m, 2H), 2.11-1.98 | |
| | (m, 1H), 1.14-1.06 (m, 2H), 1.00 (d, J =8 Hz 2H). | |
| 225 | 1H NMR (400 MHz, CD2Cl2) δ 8.40 (d, J = 5.3 Hz, 1H), 7.68 (s, 1H), 7.67 | 462.2 |
| | (d, J = 2.9 Hz, 1H), 7.53-7.47 (m, 1H), 7.35 (dd, J = 8.2, 1.9 Hz, 1H), | |
| | 7.33-7.26 (m, 2H), 7.21 (s, 1H), 4.57 (dd, J = 11.6, 1.5 Hz, 1H), 4.37-4.30 | |
| | (m, 1H), 3.86-3.77 (m, 1H), 3.40-3.31 (m, 1H), 2.68 (s, 3H), 2.57 (s, | |
| | 3H), 2.39 (s, 3H), 2.37-2.30 (m, 1H), 2.10-2.03 (m, 2H), 1.86-1.75 (m, | |
| | 1H). 19F NMR (376 MHz, CD2Cl2) δ-112.30 (s). | |
| 226 | 1H NMR (400 MHz, CD2Cl2) δ 8.39 (d, J = 5.1 Hz, 1H), 7.67 (s, 2H), | 446.2 |
| | 7.58-7.50 (m, 1H), 7.20 (s, 1H), 7.14-7.07 (m, 2H), 7.02 (td, J = 9.4, 2.4 Hz, | |
| | 1H), 4.53 (dd, J = 11.1, 1.3 Hz, 1H), 4.32 (dt, J = 5.9, 3.2 Hz, 1H), 3.85-3.77 | |
| | (m, 1H), 3.39-3.30 (m, 1H), 2.68 (s, 3H), 2.50 (s, 3H), 2.39 (s, 3H), | |
| | 2.33 (dd, J = 8.6, 6.5 Hz, 1H), 2.10-2.03 (m, 2H), 1.86-1.75 (m, 1H). 19F | |
| | NMR (376 MHz, CD2Cl2) δ-109.57 (s), -110.63 (s). | |
| 227 | 1H NMR (400 MHz, CDCl3) δ 8.02-7.75 (m, 1H), 7.68-7.49 (m, 1H), 7.45 | 477.0 |
| | (dd, J = 14.0, 6.0 Hz, 3H), 7.25 (s, 1H), 7.22 (d, J = 2.0 Hz, 1H), 4.54 (dd, J = | |
| | 11.1, 1.7 Hz, 1H), 4.25 (dd, J = 8.4, 2.7 Hz, 1H), 3.84-3.73 (m, 1H), 3.56 (tt, | |
| | J = 7.3, 3.8 Hz, 1H), 3.13 (dd, J = 9.7, 6.3 Hz, 1H), 2.72 (s, 3H), 2.64 (s, 3H), | |
| | 2.24 (d, J = 13.4 Hz, 1H), 2.03-1.89 (m, 3H), 1.16-1.05 (m, 2H), 0.99 (q, | |
| | J = 7.3 Hz, 2H). | |
| 228 | 1H NMR (400 MHz, CDCl3) δ 7.88 (d, J = 1.7 Hz, 1H), 7.56 (s, 1H), | 477.0 |
| | 7.50-7.40 (m, 3H), 7.25 (s, 1H), 7.23 (d, J = 1.9 Hz, 1H), 4.54 (d, J = 9.5 Hz, | |
| | 1H), 4.25 (d, J = 11.1 Hz, 1H), 3.85-3.73 (m, 1H), 3.56 (tt, J = 7.4, 3.9 Hz, 1H), | |
| | 3.24-3.05 (m, 1H), 2.72 (s, 3H), 2.64 (s, 3H), 2.24 (d, J = 12.8 Hz, 1H), | |
| | 2.01-1.89 (m, 3H), 1.13-1.04 (m, 2H), 0.99 (q, J = 7.3 Hz, 2H). | |
| 229 | | |
| 230 | | |
| 231 | 1H NMR (DMSO-d6, 400 MHz): δH 7.88 (1H, d, J = 8.4 Hz), 7.72 (1H, s), | 447.3 |
| | 7.61 (1H, d, J = 7.8 Hz), 7.55 (1H, s), 7.52 (1H, d, J = 10.1 Hz), 7.45 (1H, | |
| | d, J = 8.4 Hz), 7.38 (1H, s), 7.32 (1H, d, J = 8.7 Hz), 4.47 (1H, d, J = 11.1 | |
| | Hz), 4.09 (1H, d, J = 11.2 Hz), 3.65 (3H, br s), 3.58 (1H, s), 2.69 (3H, s), 2.19 | |
| | (1H, d, J = 13.1 Hz), 1.94 (3H, d, J = 10.0 Hz), 1.82 (1H, d, J = 60.5 Hz), 0.98 | |
| | (2H, s), 0.90 (2H, d, J = 7.1 Hz). | |
| 232 | 1H NMR (DMSO-d6, 400 MHz): δH 7.73 (1H, s), 7.67 (1H, s), 7.58-7.64 | 461.2 |
| | (1H, m), 7.48-7.54 (2H, m), 7.39 (1H, s), 7.32 (1H, td, J = 8.5, 2.5 Hz), 4.48 | |
| | (1H, d, J = 11.1 Hz), 4.10 (1H, d, J = 11.3 Hz), 3.62-3.71 (2H, m), 2.65 | |
| | (3H, s), 2.38 (3H, s), 2.20 (1H, d, J = 13.3 Hz), 1.89-1.98 (3H, m), 0.98-1.02 | |
| | (2H, m), 0.88-0.95 (2H, m). | |
| 233 | 1H NMR (DMSO-d6, 400 MHz): ?H 7.73 (1H, s), 7.68-7.70 (2H, m), 7.58 | 477.2 |
| | (1H, t, J = 7.9 Hz), 7.52 (3H, s), 7.39 (1H, s), 4.48 (1H, d, J = 11.2 Hz), | |
| | 4.10 (1H, d, J = 11.1 Hz), 3.65-3.71 (2H, m), 3.33 (1H, s), 2.65 (4H, s), 2.38 | |
| | (4H, s), 2.20 (1H, d, J = 13.2 Hz), 1.89-1.98 (3H, m), 0.99 (2H, s), 0.91 (2H, | |
| | d, J = 6.8 Hz). | |
| 236 | ¹H NMR (400 MHz, CDCl3) δ 7.58-7.44 (m, 1H), 7.34-7.29 (m, 3H), 5.04 | 477.2 |
| | (t, J = 4.8 Hz, 2H), 3.92 (tdd, J = 19.6, 11.6, 3.6 Hz, 1H), 3.63-3.41 (m, 2H), | |
| | 2.82 (s, 2H), 2.69 (s, 3H), 2.61-2.48 (m, 3H), 2.41-2.22 (m, 1H), 2.07 (d, | |
| | J = 4.4 Hz, 3H), 1.15-1.06 (m, 2H), 1.01 (dd, J = 10.2, 2.9 Hz, 2H). | |
| 237 | ¹H NMR (400 MHz, CDCl3) δ 7.56-7.44 (m, 4H), 7.34-7.28 (m, 2H), 4.55 | 477.2 |
| | (dd, J = 11.2, 2.0 Hz, 1H), 4.26 (dd, J = 11.5, 2.9 Hz, 1H), 3.80 (td, J = 12.0, | |
| | 2.3 Hz, 1H), 3.55 (ddd, J = 11.1, 7.4, 3.8 Hz, 1H), 3.38 (ddd, J = 12.1, 8.3, | |
| | 3.9 Hz, 1H), 2.81 (s, 3H), 2.68 (s, 3H), 2.31 (d, J = 12.8 Hz, 1H), 2.22-1.97 | |
| | (m, 3H), 1.10-1.03 (m, 2H), 0.98 (t, J = 6.3 Hz, 2H). | |

### Example A2-2: Synthesis of Exemplary Compounds

### Synthesis of I-1076 and I-1081

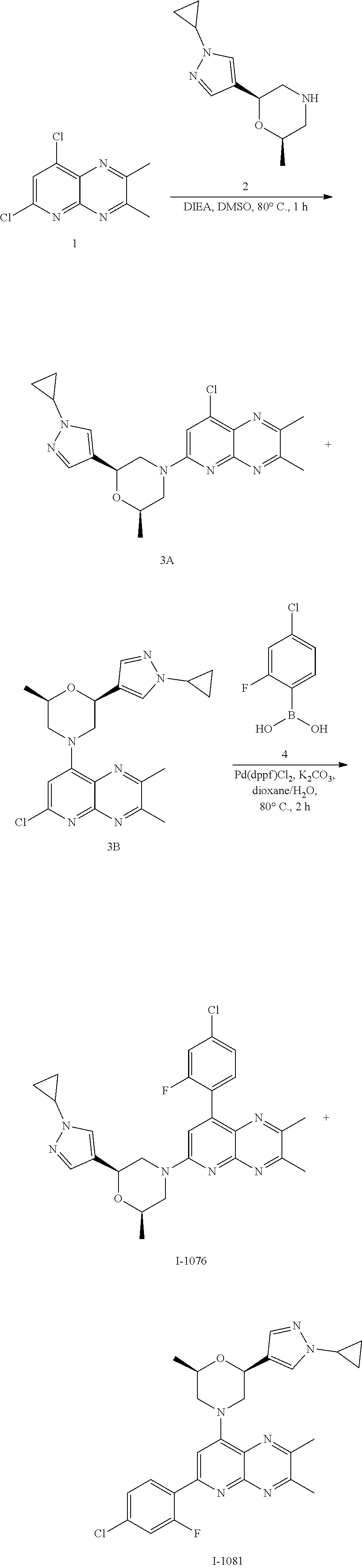

Step 1: A mixture of 6,8-dichloro-2,3-dimethyl-pyrido[2,3-b]pyrazine (1.00 eq, 400 mg, 1.75 mmol), (2S,6R)-2-(1-cyclopropylpyrazol-4-yl)-6-methyl-morpholine (1.50 eq, 545 mg, 2.63 mmol) and DIEA (3.00 eq, 0.87 mL, 5.26 mmol) in DMSO (8 mL) was stirred at 80° C. for 1 h. LCMS showed starting material was consumed completely and desired mass was detected (31%, 399.3 [M+H]⁺, ESI⁺ pos). The crude product was purified by reversed-phase HPLC (0.1% FA condition) to give the mixture of (2S,6R)-4-(8-chloro-2,3-dimethyl-pyrido[2,3-b]pyrazin-6-yl)-2-(1-cyclopropylpyrazol-4-yl)-6-methyl-morpholine 3A 150 mg, 0.376 mmol, 21.44% yield) and (2S,6R)-4-(6-chloro-2,3-dimethyl-pyrido[2,3-b]pyrazin-8-yl)-2-(1-cyclopropylpyrazol-4-yl)-6-methyl-morpholine 3B (150 mg, 0.376 mmol, 21% yield) as yellow oil. LCMS (M+H)⁺=398.9; purity=88% (220 nm). Retention time=0.873 min.

Step 2: A mixture of (2S,6R)-4-(8-chloro-2,3-dimethyl-pyrido[2,3-b]pyrazin-6-yl)-2-(1-cyclopropylpyrazol-4-yl)-6-methyl-morpholine (1.00 eq, 50 mg, 0.125 mmol) and (2S,6R)-4-(6-chloro-2,3-dimethyl-pyrido[2,3-b]pyrazin-8-yl)-2-(1-cyclopropylpyrazol-4-yl)-6-methyl-morpholine (1.00 eq, 50 mg, 0.125 mmol) in 1,4-Dioxane (1 mL) and water (0.10 mL) was added K₂CO₃ (4.00 eq, 42 mg, 0.501 mmol) and (4-chloro-2-fluoro-phenyl)boronic acid (6.00 eq, 131 mg, 0.752 mmol). Then [1,1′-Bis(diphenylphosphino)ferrocene]dichloropalladium(II) (0.100 eq, 9.2 mg, 0.0125 mmol) was added under N₂. The mixture was stirred at 80° C. for 2 h. LCMS showed starting material was consumed completely and two peaks with desired mass was detected (20% and 39%, 493.2 [M+H]⁺, ESI⁺ pos). The mixture was concentrated under reduced pressure to get the crude residue. The residue was purified by prep-HPLC (water-FA)-ACN, Phenomenex Luna C18 150*25 mm*10 um) and lyophilized to give the two crude products. The crude product 1 was purified by prep-TLC (SiO₂, Petroleum ether/Ethyl acetate=0:1, R_{f}=0.4) and lyophilized to give (2S,6R)-4-[8-(4-chloro-2-fluoro-phenyl)-2,3-dimethyl-pyrido[2,3-b]pyrazin-6-yl]-2-(1-cyclopropylpyrazol-4-yl)-6-methyl-morpholine (31 mg, 0.0627 mmol, 50% yield) as yellow solid. The crude product 2 was purified by prep-TLC (SiO₂, Petroleum ether/Ethyl acetate=0:1, R_{f}=0.4) and lyophilized to give (2S,6R)-4-[6-(4-chloro-2-fluoro-phenyl)-2,3-dimethyl-pyrido[2,3-b]pyrazin-8-yl]-2-(1-cyclopropylpyrazol-4-yl)-6-methyl-morpholine, I-1081 (32 mg, 0.0645 mmol, 51% yield) as yellow solid. LCMS: (M+H)⁺=493.3; purity=100% (220 nm). Retention time=0.946 min. ¹H NMR (400 MHz, DMSO-d6) δ ppm 0.91-1.05 (m, 4H) 1.23 (d, J=6.24 Hz, 3H) 2.47-2.48 (m, 3H) 2.59 (s, 3H) 2.68-2.75 (m, 1H) 2.98 (dd, J=12.78, 11.43 Hz, 1H) 3.69 (tt, J=7.32, 3.74 Hz, 1H) 3.73-3.81 (m, 1H) 4.52-4.57 (m, 1H) 4.57-4.66 (m, 2H) 7.44 (dd, J=8.25, 1.90 Hz, 1H) 7.48 (s, 1H) 7.54 (s, 1H) 7.55-7.61 (m, 2H) 7.84 (s, 1H). Peak 2, LCMS: (M+H)⁺=493.3; purity=100% (220 nm). Retention time=0.853 min. ¹H NMR (400 MHz, DMSO-d6) δ ppm 0.90-1.06 (m, 4H) 1.16-1.25 (m, 3H) 2.68 (d, J=3.42 Hz, 6H) 2.77-2.90 (m, 1H) 3.00-3.15 (m, 1H) 3.69 (dt, J=7.27, 3.58 Hz, 1H) 3.94-4.07 (m, 1H) 4.42 (br d, J=12.10 Hz, 1H) 4.58 (br d, J=12.59 Hz, 1H) 4.79 (dd, J=10.64, 2.08 Hz, 1H) 7.33 (s, 1H) 7.43-7.51 (m, 2H) 7.61 (dd, J=10.94, 1.90 Hz, 1H) 7.82 (s, 1H) 8.02 (t, J=8.56 Hz, 1H).

### Synthesis of I-1086

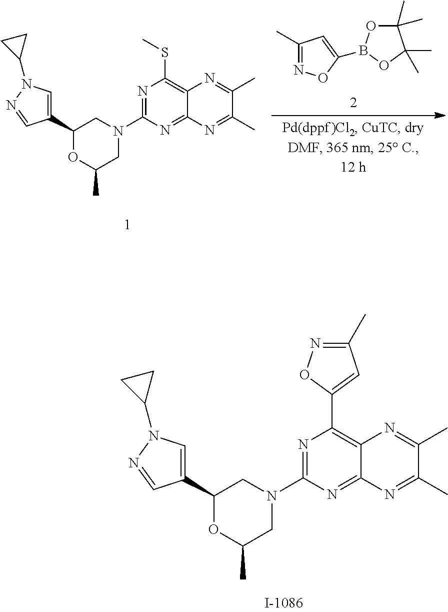

Step 1: A solution containing 3-methyl-5-(4,4,5,5-tetramethyl-1,3,2-dioxaborolan-2-yl)isoxazole (3.00 eq, 152 mg, 0.72 mmol), (2S,6R)-2-(1-cyclopropylpyrazol-4-yl)-4-(6,7-dimethyl-4-methylsulfanyl-pteridin-2-yl)-6-methyl-morpholine (1.00 eq, 100 mg, 0.24 mmol), CuTC (2.20 eq, 102 mg, 0.53 mmol) and Pd(dppf)Cl₂·CH₂Cl₂ (0.30 eq, 53 mg, 0.07 mmol) in dry DMF (5 mL) was flushed with argon for 3 min. The brown suspension was irradiated (365 nm) under argon for 8 h. LCMS showed starting material consumed and desired product (55%, Rt: 0.933 min; [M+H]⁺=447.4 at 220 nm) was detected. The reaction mixture was poured into water (20 mL), extracted with EtOAc (20 mL three times). The combined organic phase was washed by brine (20 mL), dried by Na₂SO₄ to give a crude product. The product was purified by prep-HPLC (Column: Phenomenex Synergi Polar-RP 100*25 mm*4 um; Condition: Water (TFA)-ACN) and lyophilized to give (2S,6R)-2-(1-cyclopropylpyrazol-4-yl)-4-[6,7-dimethyl-4-(3-methylisoxazol-5-yl)pteridin-2-yl]-6-methyl-morpholine (69 mg, 0.15 mmol, 63% yield) as yellow solid. [M+H]⁺=447.4. Retention time=0.933 min. LC-MS. Rt: 0.651 min, m/z: 447.1 [M+H]⁺. 100% purity at 220 nm. ¹H NMR (400 MHz, CDCl3) δ=7.73 (s, 1H), 7.62 (s, 1H), 7.57 (s, 1H), 5.15 (br d, J=13.1 Hz, 1H), 5.11-4.98 (m, 1H), 4.62 (dd, J=2.6, 10.9 Hz, 1H), 3.90-3.77 (m, 1H), 3.61 (tt, J=3.7, 7.3 Hz, 1H), 3.17-3.04 (m, 1H), 2.88 (br t, J=12.1 Hz, 1H), 2.74 (s, 3H), 2.72 (s, 3H), 2.48 (s, 3H), 1.36 (br d, J=6.0 Hz, 3H), 1.14 (br d, J=2.8 Hz, 2H), 1.08-1.02 (m, 2H). ¹⁹F NMR (376 MHz, CDCl₃) δ=−75.99 (s, 1F).

### Synthesis of Compound I-1096

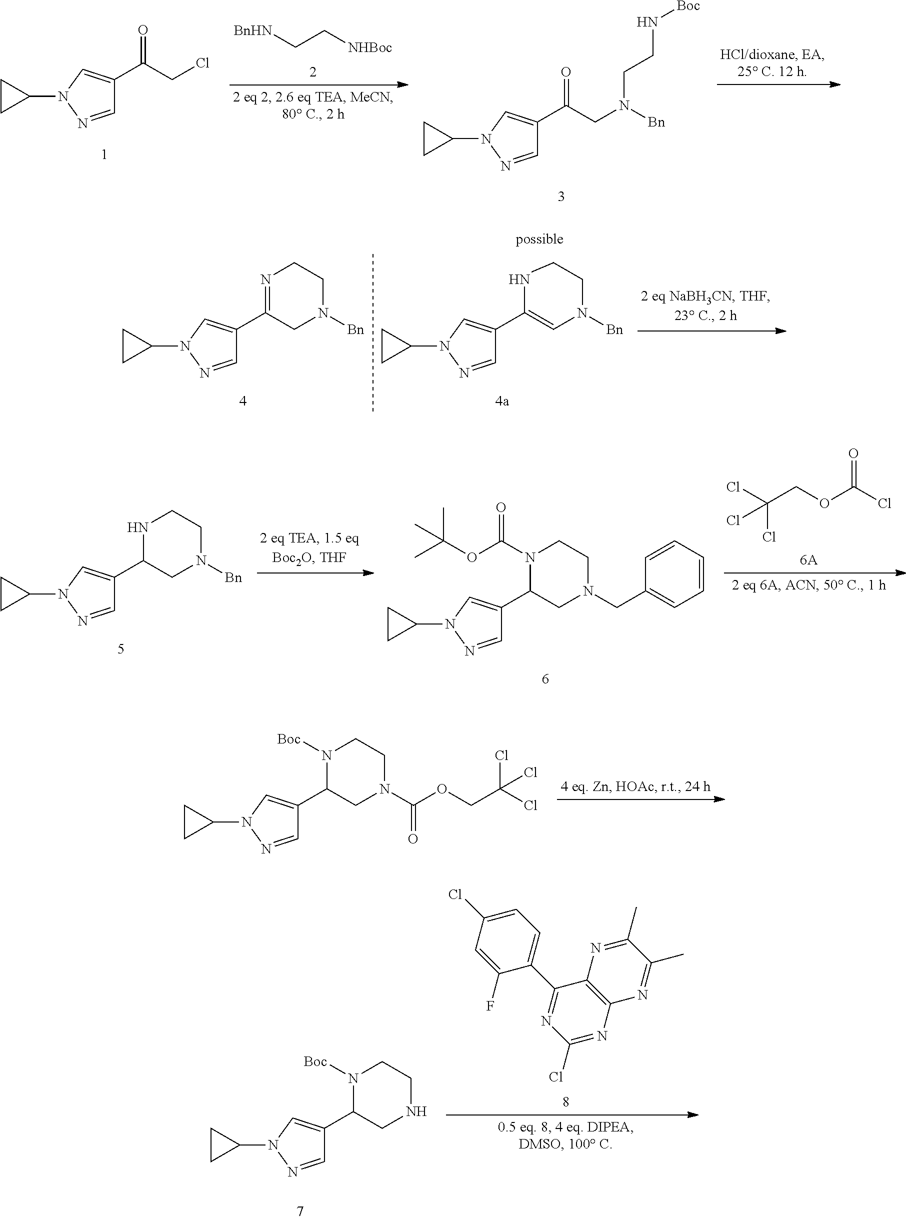

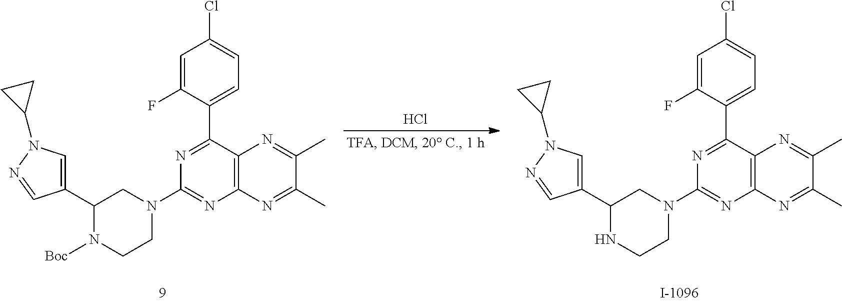 [SMILES: Cc1nc2nc(N3CCN(C(=O)OC(C)(C)C)C(c4cnn(C5CC5)c4)C3)nc(-c3ccc(Cl)cc3F)c2nc1C ; Cl ; Cc1nc2nc(N3CCNC(c4cnn(C5CC5)c4)C3)nc(-c3ccc(Cl)cc3F)c2nc1C]

Step 1: To a solution of 2-chloro-1-(1-cyclopropylpyrazol-4-yl)ethanone (1.00 eq, 700 mg, 3.79 mmol) and tert-butyl N-[2-(benzylamino)ethyl]carbamate (2.00 eq, 1898 mg, 7.58 mmol) in MeCN (30 mL) was added TEA (2.60 eq, 998 mg, 9.86 mmol), the mixture stirred at 80° C. for 1 h. LCMS showed the starting material was consumed. The mixture was poured into water. The aqueous layer was extracted with EA (100 mL) three times. The combined organic layers were washed with brine (100*5) mL and dried over Na₂SO₄.The crude product was purified by TLC (PE:EtOAc=1: 1) Rf=0.5. Tert-butyl N-[2-[benzyl-[2-(1-cyclopropylpyrazol-4-yl)-2-oxo-ethyl] amino] ethyl] carbamate (1000 mg, 2.26 mmol, 60% yield) obtained as yellow oil. 1H NMR (400 MHz, CDCl3) δ=7.90 (s, 1H), 7.84 (s, 1H), 7.32 (s, 5H), 5.21 (br s, 1H), 3.76 (s, 2H), 3.64 (s, 2H), 3.62-3.57 (m, 1H), 3.24 (br d, J=5.5 Hz, 2H), 2.76 (t, J=5.9 Hz, 2H), 1.45 (s, 9H), 1.16-1.02 (m, 4H).

Step 2: To a solution was tert-butyl N-[2-[benzyl-[2-(1-cyclopropylpyrazol-4-yl)-2-oxo-ethyl]amino]ethyl]carbamate (1.00 eq, 300 mg, 0.753 mmol) in HCl 4M in dioxanes (1.00 eq, 2.0 mL, 0.753 mmol) and Ethyl acetate (6 mL), the mixture was stirred at 25° C. for 12 h. LCMS showed no starting material remained. Yellow solid was precipitated. The mixture was added PE (10 mL) and stirred at 25° C. for 0.5 h, The mixture was filtered and the filter cake was washed with PE to give a crude product. 4-benzyl-6-(1-cyclopropylpyrazol-4-yl)-3, 5-dihydro-2H-pyrazine (200 mg, 0.713 mmol, 95% yield) obtained as yellow solid. MS (ESI): m/z=281.3 [M+H]+.

Step 3: To a solution of 4-benzyl-6-(1-cyclopropylpyrazol-4-yl)-3,5-dihydro-2H-pyrazine (1.00 eq, 100 mg, 0.357 mmol) in THF (10 mL) was added sodium cyanoborohydride (2.00 eq, 45 mg, 0.713 mmol) at 25° C., the mixture stirred at 25° C. for 1 h. LCMS showed some starting material remained, added more sodium cyanoborohydride (3.00 eq, 67 mg, 1.07 mmol). After 1 hour, LCMS showed the starting material was remained, the starting material was formed. After 12 hours, LCMS showed the reaction was completed. The crude product was used directly in the next step without further purification. 1-benzyl-3-(1-cyclopropylpyrazol-4-yl) piperazine (100 mg, 0.269 mmol, 75% yield) was obtained as yellow solid. MS (ESI): m/z=283.3 [M+H]+.ESI+.

Step 4: To a solution of 1-benzyl-3-(1-cyclopropylpyrazol-4-yl)piperazine (1.00 eq, 100 mg, 0.354 mmol) in THF (10 mL) was added TEA (2.00 eq, 0.061 mL, 0.708 mmol) and Boc₂O (1.50 eq, 116 mg, 0.531 mmol), the mixture stirred at 25° C. for 3 h. LCMS showed the completed reaction. The mixture was poured into water. The aqueous layer was extracted with EA (50 mL) three times. The combined organic layers were washed with brine (50*5) mL and dried over Na₂SO₄.The crude product was purified by prep-TLC (PE:EtOAc=1:1), desired product Rf=0.5. Tert-butyl 4-benzyl-2-(1-cyclopropylpyrazol-4-yl) piperazine-1-carboxylate (70 mg, 0.165 mmol, 47% yield) obtained as colorless oil, MS (ESI): m/z=383.3 [M+H]⁺.

Step 5: To a solution of tert-butyl 4-benzyl-2-(1-cyclopropylpyrazol-4-yl)piperazine-1-carboxylate (1.00 eq, 50 mg, 0.131 mmol) in MeCN (1 mL) was added 2,2,2-trichloroethyl carbonochloridate (1.00 eq, 27 mg, 0.131 mmol), the mixture was stirred at 50° C. for 1 h. LCMS showed the reaction was complete. The residue was purified by preparative HPLC (column: Phenomenex luna C18 150*25 mm*10 um; mobile phase: [water(FA)-ACN]; B %: 52%-82%, 10 min) and lyophilized. O1-tert-butyl O4-(2, 2, 2-trichloroethyl) 2-(1-cyclopropylpyrazol-4-yl) piperazine-1, 4-dicarboxylate (35 mg, 0.0599 mmol, 45.79% yield) obtained as colorless oil. LCMS. MS (ESI): m/z=467.0 [M+H]+

Step 6: To a solution of 01-tert-butyl 04-(2,2,2-trichloroethyl) 2-(1-cyclopropylpyrazol-4-yl)piperazine-1,4-dicarboxylate (1.00 eq, 35 mg, 0.0748 mmol) in acetic acid (2 mL) was added Zinc powder (2.04 eq, 10 mg, 0.153 mmol), The mixture was stirred at 20° C. for 2 h under N2.LCMS showed the starting material was remained. The reaction was stirred at 20° C. for 12 h. LCMS showed no starting material remained. Zinc powder (2.04 eq, 10 mg, 0.153 mmol) was added and the mixture was stirred at 20° C. for 2 h. LCMS showed the desired product was formed. The mixture was filtered and the filter cake was poured into 1 N HCl (50 ml) stirred until no gas was produced. The filter liquor was adjusted PH to 7.0 by NH₃·H₂O. The aqueous layer was extracted with DCM:MeOH=10:1 (30 mL) three times and dried over Na₂SO₄. Tert-butyl 2-(1-cyclopropylpyrazol-4-yl) piperazine-1-carboxylate (20 mg, 0.0417 mmol, 55.77% yield) obtained as colorless oil. MS (ESI): m/z=293.3 [M+H]+.

Step 7: To a solution of tert-butyl 2-(1-cyclopropylpyrazol-4-yl)piperazine-1-carboxylate (1.00 eq, 20 mg, 0.0684 mmol) and 2-chloro-4-(4-chloro-2-fluoro-phenyl)-6,7-dimethyl-pteridine (0.500 eq, 11 mg, 0.0342 mmol) in DMSO (2 mL) was added DIPEA (4.00 eq, 0.048 mL, 0.274 mmol), the mixture was stirred at 100° C. for 30 min. LCMS showed the reaction was completed. The mixture was poured into Water, the aqueous layer was extracted with EA (50 mL) three times. The combined organic layers were washed with brine (50×5) mL and dried over Na₂SO₄.The crude product was used directly in the next step without further purification. Tert-butyl 4-[4-(4-chloro-2-fluoro-phenyl)-6,7-dimethyl-pteridin-2-yl]-2-(1-cyclopropylpyrazol-4-yl) piperazine-1-carboxylate (20 mg, 0.0242 mmol, 35.34% yield) obtained as yellow solid. MS (ESI): m/z=579.4 [M+H]⁺.

Step 8: To a solution of tert-butyl 4-[4-(4-chloro-2-fluoro-phenyl)-6,7-dimethyl-pteridin-2-yl]-2-(1-cyclopropylpyrazol-4-yl)piperazine-1-carboxylate (1.00 eq, 20 mg, 0.0345 mmol) in DCM (1 mL) was added TFA (189 eq, 0.50 mL, 6.53 mmol), the mixture was stirred 20° C. for 1 h. LCMS showed the reaction was completed. The mixture pH was adjusted to 7 by NH3 H2O and concentrated to give a crude product. The residue was purified by preparative HPLC (column: Phenomenex Luna C18, 150*25 mm*10 um; mobile phase: [water (FA)-ACN]; B %: 17%-47%, 10 min) and lyophilized. [00480] 4-(4-chloro-2-fluoro-phenyl)-2-[3-(1-cyclopropylpyrazol-4-yl) piperazin-1-yl]-6, 7-dimethyl-pteridine (2.0 mg, 0.00384 mmol, 11.11% yield) was obtained as yellow solid. MS (ESI): m/z=479.1 [M+H]⁺. 1H NMR (400 MHz, CDCl3) δ=7.65 (t, J=7.8 Hz, 1H), 7.51 (d, J=3.8 Hz, 2H), 7.29 (br d, J=8.3 Hz, 2H), 5.02-5.01 (m, 1H), 5.12-4.99 (m, 1H), 4.95 (br d, J=13.1 Hz, 1H), 3.97-3.84 (m, 1H), 3.57 (br s, 1H), 3.30-3.08 (m, 3H), 3.34-3.07 (m, 4H), 3.06-2.96 (m, 1H), 2.70 (s, 3H), 2.60-2.56 (m, 3H), 1.13-1.08 (m, 2H), 1.00 (br d, J=6.6 Hz, 2H).

### Synthesis of Compounds I-1099

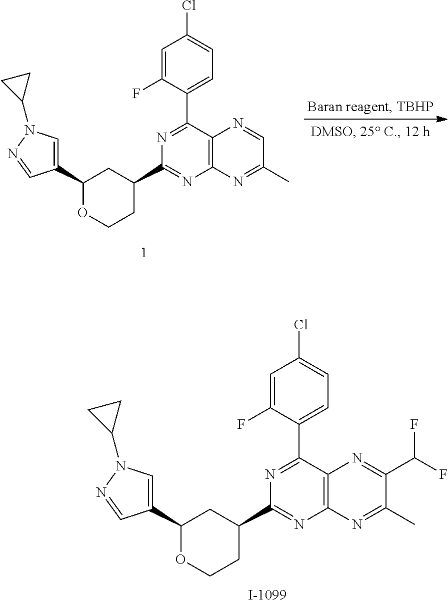 [SMILES: Cc1cnc2c(-c3ccc(Cl)cc3F)nc([C@H]3CCO[C@@H](c4cnn(C5CC5)c4)C3)nc2n1 ; Cc1nc2nc([C@H]3CCO[C@@H](c4cnn(C5CC5)c4)C3)nc(-c3ccc(Cl)cc3F)c2nc1C(F)F]

To a solution of 4-(4-chloro-2-fluoro-phenyl)-2-[(2R,4S)-2-(1-cyclopropylpyrazol-4-yl)tetrahydropyran-4-yl]-7-methyl-pteridine (1.00 eq, 250 mg, 0.538 mmol) and zinc difluoromethanesulfinate (4.00 eq, 632 mg, 2.15 mmol) in DMSO (4 mL) at 25° C. was added tert-butylhydroperoxide (6.00 eq, 415 mg, 3.23 mmol) with vigorous stirring and bubbled with N₂ for 30 seconds. The reaction solution was stirred at 25° C. for 12 hrs. LCMS showed 49% of reactant was consumed and 30% of desired mass was detected, the reaction solution was purified with reversed column (FA) and lyophilized to give the crude, which was then purified with prep-HPLC (FA) and lyophilized to give 4-(4-chloro-2-fluoro-phenyl)-2-[(2R,4S)-2-(1-cyclopropylpyrazol-4-yl)tetrahydropyran-4-yl]-6-(difluoromethyl)-7-methyl-pteridine (19 mg, 0.0367 mmol, 6.82% yield) as yellow solid. LCMS (M+H)+=514.9; purity=98.4% (220 nm). Retention time=1.025 min. 1H NMR (400 MHz, CDCl₃) δ=7.78-7.69 (m, 1H), 7.52 (d, J=1.2 Hz, 2H), 7.40 (dd, J=1.7, 8.3 Hz, 1H), 7.36-7.30 (m, 1H), 6.94-6.60 (m, 1H), 4.67-4.53 (m, 1H), 4.35-4.25 (m, 1H), 3.92-3.79 (m, 1H), 3.69-3.49 (m, 2H), 3.08 (s, 3H), 2.56-2.41 (m, 1H), 2.31-2.13 (m, 3H), 1.15-1.08 (m, 2H), 1.04-0.97 (m, 2H).

### Synthesis of Compound I-1104

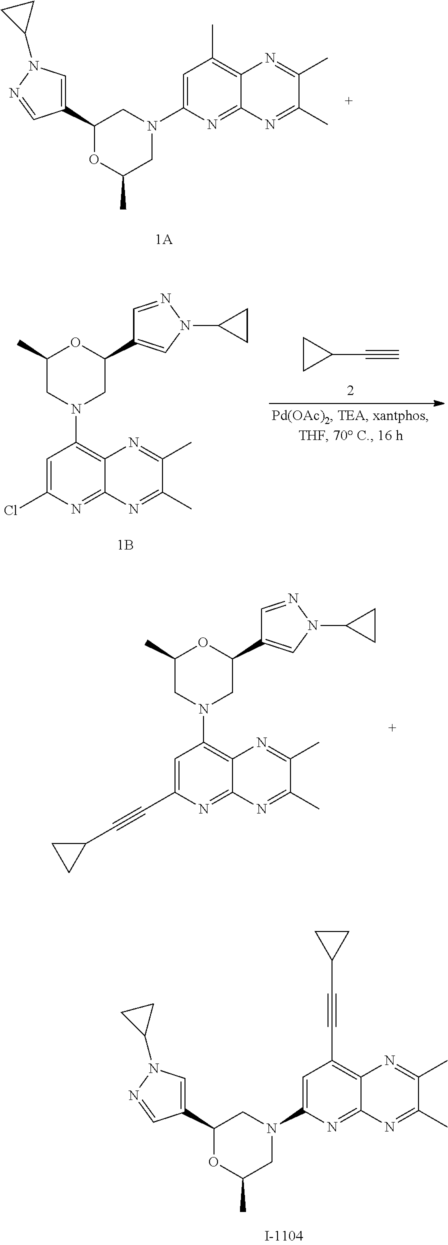

To a solution of (2S,6R)-4-(8-chloro-2,3-dimethylpyrido[2,3-b]pyrazin-6-yl)-2-(1-cyclopropyl-1H-pyrazol-4-yl)-6-methylmorpholine and (2S,6R)-4-(6-chloro-2,3-dimethylpyrido[2,3-b]pyrazin-8-yl)-2-(1-cyclopropyl-1H-pyrazol-4-yl)-6-methylmorpholine (1.00 eq, 90 mg, 0.226 mmol), ethynylcyclopropane (3.00 eq, 45 mg, 0.677 mmol) and xantphos (0.1000 eq, 13 mg, 0.0226 mmol) in THF (1 mL) and TEA (2.00 eq, 0.063 mL, 0.451 mmol) was added Pd(OAc)₂ (0.0500 eq, 2.5 mg, 0.0113 mmol) under atmosphere of N₂. Then the mixture was stirred at 70° C. for 16 h. LCMS showed starting material was consumed completely and desired mass was detected (14% and 20%, 429.3 [M+H]⁺, ESI⁺ pos). The mixture was filtered and the filtrate was concentrated under reduced pressure to get the crude residue. The residue was purified by prep-HPLC (water (FA)-ACN, Phenomenex luna C18 150*25 mm*10 um) and lyophilized to give (2S,6R)-4-[6-(2-cyclopropylethynyl)-2,3-dimethyl-pyrido[2,3-b]pyrazin-8-yl]-2-(1-cyclopropylpyrazol-4-yl)-6-methyl-morpholine (13 mg, 0.0291 mmol, 12.90% yield) as yellow solid. The crude product was purified by prep-TLC (SiO₂, Petroleum ether/Ethyl acetate=0:1, rf=0.3) to give (2S,6R)-4-[8-(2-cyclopropylethynyl)-2,3-dimethyl-pyrido[2,3-b]pyrazin-6-yl]-2-(1-cyclopropylpyrazol-4-yl)-6-methyl-morpholine (7.9 mg, 0.0181 mmol, 8.02% yield) as yellow solid. (M+H)⁺=429.2; purity=98.2% (220 nm). Retention time=0.836 min. ¹H NMR (400 MHz, DMSO-d6) δ ppm0.80-0.86 (m, 2H) 0.91-1.03 (m, 6H) 1.19 (d, J=6.25 Hz, 3H) 1.62 (tt, J=8.27, 4.99 Hz, 1H) 2.63 (d, J=3.75 Hz, 6H) 2.74 (dd, J=12.01, 10.76 Hz, 1H) 2.95-3.05 (m, 1H) 3.69 (tt, J=7.38, 3.69 Hz, 1H) 3.90-4.00 (m, 1H) 4.38 (br d, J=12.38 Hz, 1H) 4.51 (br d, J=12.38 Hz, 1H) 4.73 (dd, J=10.57, 2.31 Hz, 1H) 6.99 (s, 1H) 7.45 (s, 1H) 7.82 (s, 1H). LCMS: (M+H)⁺=429.2; purity=98.2% (220 nm). Retention time=0.903 min. ¹H NMR (400 MHz, DMSO-d6) δ ppm 0.80-0.88 (m, 2H) 0.92-1.05 (m, 6H) 1.21 (br d, J=6.11 Hz, 3H) 1.62-1.75 (m, 1H) 2.58 (d, J=2.81 Hz, 6H) 2.64-2.70 (m, 1H) 2.93 (br t, J=11.62 Hz, 1H) 3.64-3.78 (m, 2H) 4.45-4.62 (m, 3H) 7.48 (s, 1H) 7.52 (s, 1H) 7.84 (s, 1H).

### Synthesis of 1-1119

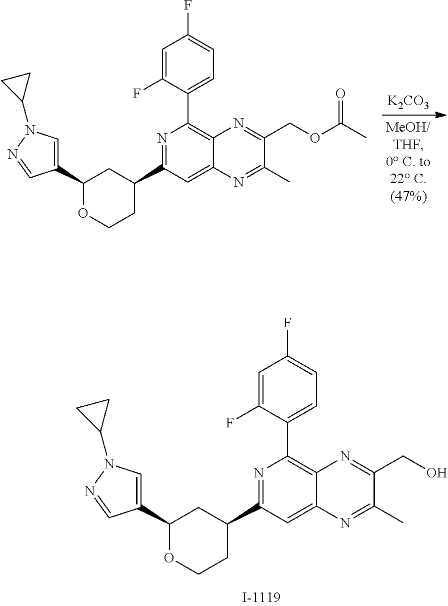

A glass vial equipped with a Teflon-coated magnetic stirring bar was charged with (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methyl acetate (1.0 equiv, 10 mg, 0.019 mmol), MeOH (0.25 mL) and THF (0.25 mL). The vial was cooled in an ice bath at 0° C. with stirring. Potassium carbonate (1.0 equiv, 2.7 mg, 0.019 mmol) was added and the reaction mixture was stirred at 0° C. for 90 min and 22° C. for 4 days. The reaction mixture was quenched with 5% citric acid (aq) (25 μL) and evaporated to dryness under reduced pressure. The crude residual material was purified by silica gel flash chromatography (Pre-packed Teledyne RediSep® GOLD column, 12 g SiO₂) using an elution gradient of 0% to 10% MeOH in DCM to afford (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methanol (4.3 mg, 0.009 mmol, 47% yield) as an off-white solid. LC-MS(ESI+): Tr=1.38 min; [M+H]+ 478.2 (obs). ¹H NMR (DMSO-d₆, 400 MHz): δ_{H} 7.87 (1H, s), 7.68-7.73 (2H, m), 7.37-7.43 (2H, m), 7.26 (1H, dd, J=9.8, 7.6 Hz), 5.37 (1H, t, J=5.7 Hz), 4.71 (2H, d, J=5.8 Hz), 4.49 (1H, d, J=11.1 Hz), 4.09 (1H, d, J=11.3 Hz), 3.70 (1H, t, J=11.3 Hz), 3.63-3.67 (1H, m), 2.81 (3H, s), 2.22 (1H, d, J=13.2 Hz), 1.85-1.94 (3H, m), 0.97-1.00 (2H, m), 0.89-0.95 (2H, m). All temperatures are in reported in degrees Celsius (° C.) and are uncorrected. Reagent grade chemicals and anhydrous solvent were purchased from commercial sources and unless otherwise mentioned, were used without further purification. Flash chromatography was performed on Teledyne Isco instruments using pre-packaged disposable SiO₂ stationary phase columns with eluent flow rate range of 15 to 200 mL/min, UV detection (254 and 220 nm). The analytical HPLC chromatograms were performed using an Agilent 1100 series instrument with DAD detector (190 nm to 300 nm). The mass spectra were recorded with a Waters Micromass ZQ detector at 130° C. The mass spectrometer was equipped with an electrospray ion source (ESI) operated in a positive ion mode and was set to scan between m/z 150-800 with a scan time of 0.3 s. Products and intermediates were analyzed by UPLC/MS on a Gemini-NX (5 M, 2.0×30 mm) using a low pH buffer gradient of 10% to 95% of ACN in H₂O (0.1% HCOOH) over 5 min at 1.0 mL/min for a 3.5 min run. The ¹H NMR spectra were recorded on a Varian NMR (AS 400). The chemical shifts are reported in part-per-million from a tetramethylsilane standard.

### Synthesis of Compound I-1124

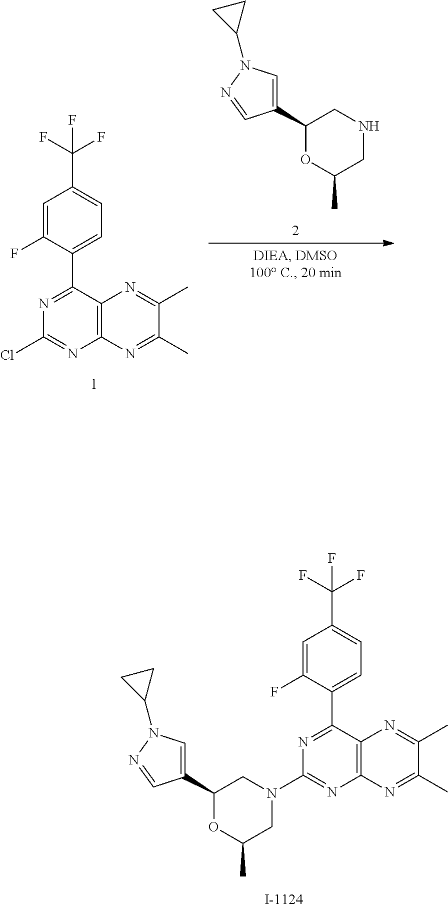 [SMILES: Cc1nc2nc(Cl)nc(-c3ccc(C(F)(F)F)cc3F)c2nc1C ; C[C@@H]1CNC[C@H](c2cnn(C3CC3)c2)O1 ; Cc1nc2nc(N3C[C@@H](C)O[C@@H](c4cnn(C5CC5)c4)C3)nc(-c3ccc(C(F)(F)F)cc3F)c2nc1C]

To a solution of 2-chloro-4-[2-fluoro-4-(trifluoromethyl)phenyl]-6,7-dimethyl-pteridine (1.00 eq, 80 mg, 0.224 mmol) and (2S,6R)-2-(1-cyclopropylpyrazol-4-yl)-6-methyl-morpholine (1.10 eq, 51 mg, 0.247 mmol) in DMSO (2 mL) was added DIEA (5.00 eq, 0.54 mL, 3.26 mmol), then the mixture was stirred at 100° C. for 20 min. LCMS (5-95AB/1.5 min): RT=0.702 min, 528.3=[M+H]⁺, ESI⁺ showed starting material was consumed completely and desired mass was detected. The reaction mixture was diluted with water 10 mL and extracted with EA (10 mL*2). The combined organic layers were dried over [Na₂SO₄], filtered and concentrated under reduced pressure to give a residue. The residue was purified by prep-HPLC (water (FA)-ACN, Phenomenex luna C18 150*25 mm, 10 um) to give (2S,6R)-2-(1-cyclopropylpyrazol-4-yl)-4-[4-[2-fluoro-4-(trifluoromethyl)phenyl]-6,7-dimethyl-pteridin-2-yl]-6-methyl-morpholine (47 mg, 0.0889 mmol, 39.65% yield) as yellow solid (single enantiomer), LCMS: (M+H)⁺=528.1; purity=99.04% (220 nm); Retention time=1.063 min. ¹H NMR (400 MHz, CDCl₃) δ ppm 1.02 (br d, J=6.25 Hz, 2H) 1.08-1.16 (m, 2H) 1.33 (d, J=6.13 Hz, 3H) 2.59 (s, 3H) 2.73 (s, 3H) 2.85 (dd, J=12.63, 11.51 Hz, 1H) 3.04-3.14 (m, 1H) 3.51-3.64 (m, 1H) 3.77-3.89 (m, 1H) 4.61 (br d, J=10.13 Hz, 1H) 4.86-5.21 (m, 2H) 7.48-7.63 (m, 4H) 7.78-7.85 (m, 1H).

### Synthesis of Compound I-1129

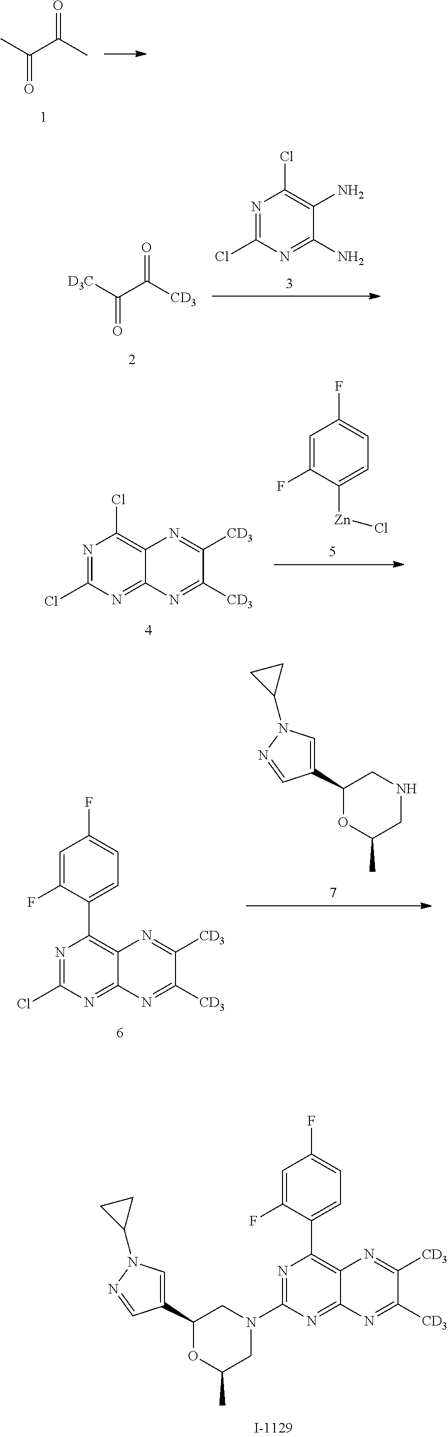

Step 1: Butane-2,3-dione was deuterated by using D₂O and D₂SO₄ for eight cycles. For each cycle, the amounts of D₂O and D₂SO₄ were adjusted depending on the amount of butanedione used in the cycle. For the first cycle, butane-2,3-dione (1.00 eq, 50.00 g, 581 mmol) in D₂O (8.61 eq, 100.00 g, 5000 mmol) was added D₂SO₄ (1.0 mL) and the mixture was stirred for 12 h at 95° C. The partially deuterated butane-2,3-dione was isolated by distillation under atmospheric pressure at 98° C. Partially deuterated butane-2,3-dione thus isolated was used without further purification in the next cycle. After the final cycle, the 1,1,1,4,4,4-hexadeuteriobutane-2,3-dione (12.50 g, 136 mmol, 68.71% yield) was isolated by distillation under atmospheric pressure at 98° C. The thus isolated was used without further purification in the next reaction. 98.92 atom % D for 1,1,1,4,4,4-hexadeuteriobutane-2,3-dione was H NMR by using MeOH as internal standard.

Step 2: To a solution of 2,6-dichloropyrimidine-4,5-diamine (1.00 eq, 180 mg, 1.01 mmol) in DCE (10 mL) was added CaSO₄ (5.00 eq, 684 mg, 5.03 mmol) and 1,1,1,4,4,4-hexadeuteriobutane-2,3-dione (1.50 eq, 139 mg, 1.51 mmol) and then the mixture was stirred for 16 h at 85° C. LCMS showed raw material was consumed completely and the major peak showed MS (233.7[M]+; ESI+, LC-RT: 0.748 min). The mixture was cooled to room temperature and diluted with acetonitrile (50 mL) and filtered through celite. The filter cake was washed with acetonitrile (20 mL×2). The combined filtrate was concentrated and purified by silica gel column (PE/EA=3/1, R_{f}=0.5) to give 2,4-dichloro-6,7-bis(trideuteriomethyl)pteridine (190 mg, 0.808 mmol, 80.37% yield) as light brown solid. A three necked was equipped with 2,4-difluoro-1-iodo-benzene (1.00 eq, 500 mg, 2.08 mmol), the flash was sealed and purged with N₂ for 3 times, THF (10 mL) was added and the solution was cooled to −40° C. with stirring, iPrMgCl—LiCl (1.3 M in THF) (1.10 eq, 1.8 mL, 2.29 mmol) was added dropwise at −40° C. and stirred for 30 min at this temperature, The reaction mixture was further cooled to −60° C. and ZnCl₂ (0.5 M in THF, 1.00 eq, 4.2 mL, 2.08 mmol) was added dropwise, the reaction solution turned into white floc, the reaction mixture was allowed to warm to room temperature gradually and stirred for 1 hr. the white floc turned into colorless solution and then use for next step.

Step 3 A sealed bottle under N₂ atmosphere was charged with 2,4-dichloro-6,7-bis(trideuteriomethyl)pteridine (1.00 eq, 40 mg, 0.170 mmol) and PdCl₂(Amphos) (0.0500 eq, 6.0 mg, 0.00851 mmol) in THF (2 mL) and purged with N₂ three times, then cooled to 0° C., chloro-(2,4-difluorophenyl)zinc (1.20 eq, 44 mg, 0.204 mmol) was added dropwise at 0° C. and warmed to 25° C., stirred for 2 h at 25° C. The reaction solution was changed from yellow to dark brown. LCMS showed raw material was consumed and the major peak showed desired MS (311.7 [M+H−1]+; ESI+). The reaction was added water (5 mL) and then extracted with EtOAc (5 mL*2) and the organics washed with 5 mL saturated brine solution. The organics were then separated and dried (Na₂SO₄) before concentration to dryness. The crude was then purified by prep-TLC (PE/EA=0/1, R_{f}=0.7) to give 2-chloro-4-(2,4-difluorophenyl)-6,7-bis(trideuteriomethyl)pteridine (25 mg, 0.0799 mmol, 46.99% yield) as white solid. 1H NMR (400 MHz, CDCl₃) δ=7.84-7.76 (m, 1H), 7.14-7.08 (m, 1H), 7.01 (ddd, J=2.4, 8.9, 9.8 Hz, 1H).

Step 4: (2S,6R)-2-(1-cyclopropylpyrazol-4-yl)-4-[4-(2,4-difluorophenyl)-6,7-bis(trideuteriomethyl)pteridin-2-yl]-6-methyl-morpholine. To a solution of 2-chloro-4-(2,4-difluorophenyl)-6,7-bis(trideuteriomethyl)pteridine (1.00 eq, 25 mg, 0.0799 mmol) and (2S,6R)-2-(1-cyclopropylpyrazol-4-yl)-6-methyl-morpholine (1.50 eq, 25 mg, 0.120 mmol) in DMSO (1 mL) was added DIEA (4.00 eq, 41 mg, 0.320 mmol) and then the mixture was stirred for 20 min at 100° C. The reaction from light-yellow to brown. LCMS showed raw material was consumed and the major peak showed desired MS-2 (482.0 [M+H]+; ESI+, LC-RT: 1.014 min).The reaction mixture was poured into water (10 mL) and then extracted with EtOAc (5 mL*2) and the organics washed with 5 ml saturated brine solution. The organics were then separated and dried (Na₂SO₄) before concentration to dryness. The crude was then purified by prep-TLC (PE/EA=0/1, R_{f}=0.5) to give the solid and then freeze-drying to give (2S,6R)-2-(1-cyclopropylpyrazol-4-yl)-4-[4-(2,4-difluorophenyl)-6,7-bis(trideuteriomethyl)pteridin-2-yl]-6-methyl-morpholine (5.3 mg, 0.0108 mmol, 13.53% yield) as yellow solid which was H NMR. LCMS: (M+H)⁺=481.9; purity=98.67% (UV=220 nm). Retention time=1.011 min. 1H NMR (400 MHz, CDCl3) δ=5.15-4.93 (m, 2H), 4.64-4.58 (m, 1H), 3.89-3.80 (m, 1H), 3.63-3.54 (m, 1H), 3.13-3.04 (m, 1H), 2.89-2.81 (m, 1H), 2.73-2.68 (m, 2H), 2.61-2.56 (m, 1H), 1.35-1.32 (m, 3H), 1.15-1.10 (m, 2H), 1.05-0.99 (m, 2H).

### Synthesis of Compound I-1144

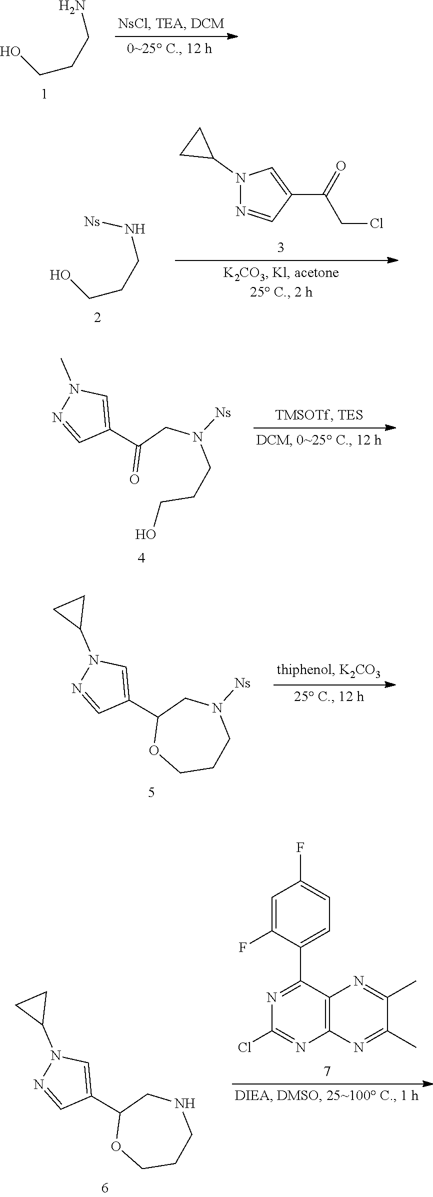

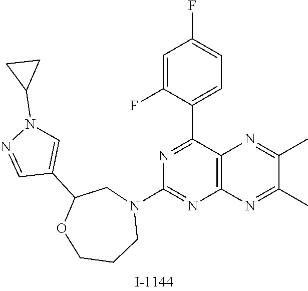 [SMILES: Cc1nc2nc(N3CCCOC(c4cnn(C5CC5)c4)C3)nc(-c3ccc(F)cc3F)c2nc1C]

Step 1: A solution of 3-aminopropan-1-ol (1.00 eq, 2000 mg, 26.6 mmol) and NsCl (1.20 eq, 7081 mg, 32.0 mmol) in DCM (40 mL) was added TEA (3.00 eq, 6.9 mL, 79.9 mmol) at 0° C. then stirred at 25° C. for 12 h. LCMS showed the starting material was consumed completely and a major peak without desired MS (85%, Rt: 0.533 min; [M+H]⁺=344.2 at 220 nm). The mixture was diluted with water (40 mL) at 0° C. and extracted with DCM (40 mL) twice. The combined organic layers were dried over Na₂SO₄. The solvent was filtered and concentrated under reduced pressure. The crude product was purified by column chromatography on silica gel (SiO₂, PE/EtOAc=1/1 to 0/1; PE/EtOAc=1/1, the desired product R_{f}=0.6) to give N-(3-hydroxypropyl)-4-nitrobenzenesulfonamide (6.20 g, 23.8 mmol, 89.46% yield) as white solid, checked by LCMS: (M−H₂O)⁺=242.7; purity=91% (220 nm). Retention time=0.396 min. ¹H NMR (400 MHz, CDCl₃) δ=8.38 (d, J=8.8 Hz, 2H), 8.07 (d, J=8.8 Hz, 2H), 5.40 (br t, J=5.4 Hz, 1H), 3.89-3.65 (m, 2H), 3.21 (q, J=6.0 Hz, 2H), 1.76 (td, J=5.8, 11.6 Hz, 2H), 1.71 (br s, 1H).

Step 2: To a solution of N-(3-hydroxypropyl)-4-nitrobenzenesulfonamide (1.50 eq, 423 mg, 1.62 mmol) and 2-chloro-1-(1-cyclopropyl-1H-pyrazol-4-yl)ethan-1-one (1.00 eq, 200 mg, 1.08 mmol) in Acetone (10 mL) was added K₂CO₃ (3.00 eq, 449 mg, 3.25 mmol), and KI (1.00 eq, 180 mg, 1.08 mmol). The mixture stirred at 25° C. for 2 h. LCMS showed the starting material was consumed and a major peak with desired mass (35%, Rt: 0.832 min; [M+H]⁺=409.2 at 220 nm). The mixture was poured into 1 N HCl (100 mL), The aqueous layer was extracted with EtOAc (100 mL) twice. The combined organic layers were dried over Na₂SO₄ and concentrated under reduced pressure to give a residue. The crude product was purified by column chromatography on silica gel eluted with PE/EtOAc=I/O to 0/1 (PE/EtOAc=1/1, the desired product R_{f}=0.5) to give N-(2-(1-cyclopropyl-1H-pyrazol-4-yl)-2-oxoethyl)-N-(3-hydroxypropyl)-4-nitrobenzenesulfonamide (280 mg, 0.617 mmol, 56.95% yield) as yellow solid, checked by LCMS, (M+H)⁺=408.9; purity=100% (220 nm). Retention time=0.614 min. H NMR (400 MHz, CDCl₃) δ=8.39-8.33 (m, 2H), 8.06-8.00 (m, 2H), 7.99 (s, 1H), 7.88 (s, 1H), 4.61 (s, 2H), 3.75 (t, J=5.6 Hz, 2H), 3.69-3.62 (m, 1H), 3.47 (t, J=6.5 Hz, 2H), 1.77 (quin, J=6.1 Hz, 2H), 1.20-1.15 (m, 2H), 1.14-1.09 (m, 2H).

Step 3: To a solution of N-(2-(1-cyclopropyl-1H-pyrazol-4-yl)-2-oxoethyl)-N-(3-hydroxypropyl)-4-nitrobenzenesulfonamide (1.00 eq, 260 mg, 0.637 mmol) in DCM (26 mL) was added TES (5.00 eq, 369 mg, 3.18 mmol). Then TMSOTf (5.00 eq, 0.58 mL, 3.18 mmol) was added at 0° C. under N₂. The mixture was stirred at 25° C. for 12 h. LCMS showed that the starting material was consumed completely and major peak with desired mass (70%, Rt: 0.590 min; [M+H]⁺=393.0 at 220 nm). The reaction mixture was poured into sat. NaHCO₃ solution (20 mL), the aqueous phase was extracted with DCM (20 mL×3). The combined organic phase was washed with brine (20 mL), dried with anhydrous Na₂SO₄, filtered and concentrated to give a crude product in vacuum. The crude product was purified by column chromatography on silica gel eluted (SiO₂, PE/EtOAc=1/0 to 0/1; PE/EtOAc=1/1, the desired product R_{f}=0.6) to afford 2-(1-cyclopropyl-1H-pyrazol-4-yl)-4-((4-nitrophenyl)sulfonyl)-1,4-oxazepane (240 mg, 0.612 mmol, 96.07% yield) as yellow solid, checked by LCMS [M+H]⁺=393.1; purity=99% (220 nm). Retention time=0.587 min. ¹H NMR (400 MHz, CDCl₃) δ=8.40-8.35 (m, 2H), 8.02-7.96 (m, 2H), 7.43 (d, J=7.8 Hz, 2H), 4.66 (dd, J=2.7, 10.0 Hz, 1H), 4.20-4.11 (m, 1H), 4.01-3.94 (m, 1H), 3.90-3.80 (m, 2H), 3.56 (td, J=3.5, 7.2 Hz, 1H), 3.12 (ddd, J=5.9, 8.2, 13.8 Hz, 1H), 2.96 (dd, J=10.0, 13.9 Hz, 1H), 2.17-2.06 (m, 2H), 1.13-1.07 (m, 2H), 1.05-0.99 (m, 2H).

Step 4: A mixture of 2-(1-cyclopropyl-1H-pyrazol-4-yl)-4-((4-nitrophenyl)sulfonyl)-1,4-oxazepane (1.00 eq, 180 mg, 0.459 mmol), K₂CO₃ (5.00 eq, 317 mg, 2.29 mmol) in MeCN (5 mL) was added thiophenol (5.00 eq, 252 mg, 2.29 mmol), then the mixture was stirred at 25° C. for 12 hrs. LCMS showed that the starting material was consumed completely and the desired mass was detected (12%, Rt: 0.559 min; [M+H]⁺=208.1 at 220 nm) and 66% of thiophenol. The reaction mixture was quenched by addition water (20 mL), and then washed with EtOAc (15 mL). The aqueous phase was lyophilized and triturated in DCM/MeOH=10/1 (15 mL). Then the mixture was filtered and the filtrate was concentrated under vacuum to give 2-(1-cyclopropyl-1H-pyrazol-4-yl)-1,4-oxazepane (90 mg, 0.434 mmol, 94.67% yield) as yellow oil, which was checked by LCMS [M+H]⁺=208.1; purity=64.6% (220 nm). Retention time=0.627 min. ¹H NMR (400 MHz, CDCl₃) δ=7.41 (d, J=5.0 Hz, 2H), 4.57 (dd, J=3.1, 9.4 Hz, 1H), 4.03 (td, J=5.5, 12.5 Hz, 1H), 3.83 (ddd, J=4.9, 8.0, 12.6 Hz, 1H), 3.55 (tt, J=3.8, 7.3 Hz, 1H), 3.28 (dd, J=3.2, 13.8 Hz, 1H), 3.14 (td, J=5.5, 13.5 Hz, 1H), 3.00-2.88 (m, 2H), 2.04-1.85 (m, 2H), 1.19-1.07 (m, 2H), 1.05-0.92 (m, 2H).

Step 5: To a solution of 2-(1-cyclopropyl-1H-pyrazol-4-yl)-1,4-oxazepane (1.00 eq, 14 mg, 0.0652 mmol) and DIEA (3.00 eq, 0.032 mL, 0.196 mmol) in DMSO (1 mL) was added 2-chloro-4-(2,4-difluorophenyl)-6,7-dimethylpteridine (1.00 eq, 20 mg, 0.0652 mmol) at 25° C. Then the reaction mixture was stirred at 100° C. for 1 h. LCMS showed 94% of desired product (94%, Rt: 0.946 min; [M+H]⁺=478.3 at 220 nm). The reaction was diluted with water (10 mL) and then extracted with ethyl acetate (15 mL×3). The combined organic layers were dried over Na₂SO₄, filtered and concentrated under reduced pressure to give a residue. The residue was purified by prep-TLC (SiO₂, PE/EtOAc=1/1, Rf=0.4) to afford 2-(1-cyclopropyl-1H-pyrazol-4-yl)-4-(4-(2,4-difluorophenyl)-6,7-dimethylpteridin-2-yl)-1,4-oxazepane (12 mg, 0.0258 mmol, 39.50% yield) as yellow solid, LCMS (5-95AB/1.5 min). [M+H]⁺=478.3; purity=100% (220 nm). Retention time=0.939 min. ¹H NMR (400 MHz, DMSO-d₆) δ ppm 0.90-0.97 (m, 2H) 1.03 (br s, 2H) 1.21 (br d, J=5.63 Hz, 3H) 2.29 (s, 3H) 2.57 (s, 3H) 2.74 (dd, J=13.13, 10.76 Hz, 1H) 3.03 (br t, J=11.88 Hz, 1H) 3.65-3.80 (m, 2H) 4.53 (br d, J=10.51 Hz, 1H) 4.67-4.84 (m, 2H) 7.27-7.37 (m, 1H) 7.44-7.55 (m, 2H) 7.58 (d, J=2.63 Hz, 1H) 7.65-7.74 (m, 1H) 7.84 (s, 1H). ¹H NMR (400 MHz, DMSO-d₆, 80° C.) δ ppm 7.83-7.71 (m, 2H), 7.45-7.32 (m, 2H), 7.26 (dt, J=2.5, 8.4 Hz, 1H), 4.82-4.65 (m, 2H), 4.51 (td, J=5.5, 13.8 Hz, 1H), 4.02 (td, J=4.8, 12.8 Hz, 1H), 3.73-3.57 (m, 3H), 3.53 (ddd, J=3.8, 8.9, 12.8 Hz, 1H), 2.66 (s, 3H), 2.53 (s, 3H), 2.16-1.92 (m, 2H), 1.04 (br s, 2H), 1.00-0.92 (m, 2H). 19F NMR (376 MHz, DMSO-d₆) δ=−107.22-−107.44 (m, 1F), -107.71 (br dd, J=9.9, 24.0 Hz, 1F).

### Synthesis of Compounds I-1147

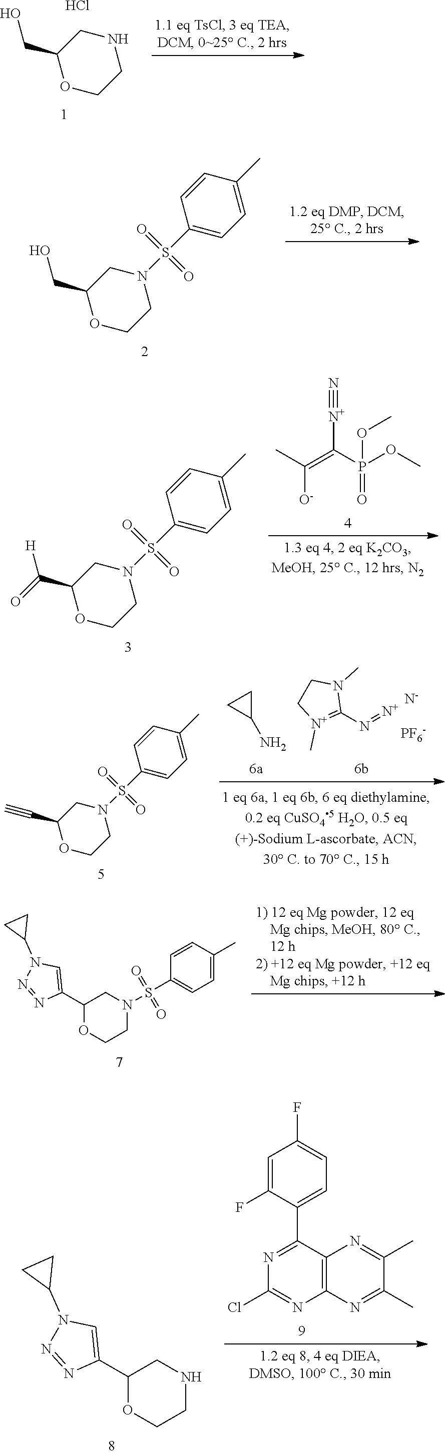

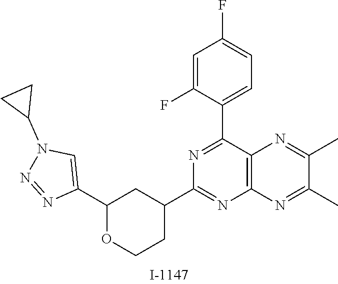 [SMILES: Cc1nc2nc(C3CCOC(c4cn(C5CC5)nn4)C3)nc(-c3ccc(F)cc3F)c2nc1C]

Step 1: To a solution of [(2R)-morpholin-2-yl]methanol hydrochloride (1.00 eq, 500 mg, 3.25 mmol) in DCM (10 mL) was added TsCl (1.20 eq, 745 mg, 3.91 mmol) dropwise at 0° C., Then the reaction mixture was stirred at 25° C. for 2 hours. LCMS (5-95AB/1.5 min): RT=0.789 min, 272.2=[M+H]⁺, ESI⁺ showed 93.6% of desired product. The reaction was diluted with water (50 mL) and then extracted with DCM (50 mL*3). The combined organic layers were washed with brine, dried over Na₂SO₄, filtered and concentrated under reduced pressure to give [(2R)-4-(p-tolylsulfonyl)morpholin-2-yl]methanol (720 mg, 2.50 mmol, 76.88% yield) as colorless oil. LCMS: Rt: 0.468 min; [M+H]⁺=272.0; 94.3% purity at 220 nm.

Step 2: To a solution of [(2R)-4-(p-tolylsulfonyl)morpholin-2-yl]methanol (1.00 eq, 4.00 g, 14.7 mmol) in DCM (60 mL) was added Dess-Martin periodinane (1.20 eq, 7503 mg, 17.7 mmol) at 0° C. Then the reaction solution was warmed to 25° C. and stirred for 16 hours. LCMS (5-95AB/1.5 min): RT=0.446 min, 270.0=[M+H]⁺, ESI⁺ showed 61% of desired product. The reaction mixture was quenched with saturated sodium thiosulfate solution (80 mL) and adjusted pH to 7-8 by sodium bicarbonate saturated solution. The mixture was extracted with ethyl acetate (80 mL*3). The combined organic layers were washed with brine (80 mL), dried over anhydrous sodium sulfate, filtered and concentrated in vacuo to give a residue. The residue was purified by flash chromatography on silica gel eluting with PE/EtOAc (2:1) (TLC, PE:EtOAc=0:1, R_{f}=0.55) to afford (2R)-4-(p-tolylsulfonyl)morpholine-2-carbaldehyde (3.10 g, 11.5 mmol, 78.08% yield) a white crystal, which ¹H NMR (400 MHz, DMSO-d6) δ ppm 2.42 (s, 3H) 3.18-3.31 (m, 1H) 3.37-3.54 (m, 3H) 3.66 (ddd, J=11.88, 9.07, 2.94 Hz, 1H) 3.81-3.89 (m, 1H) 4.22 (dd, J=8.57, 3.31 Hz, 1H) 7.48 (br d, J=8.25 Hz, 2H) 7.58-7.66 (m, 2H) 9.52 (s, 1H).

Step 3: To a solution of 1-diazo-1-dimethoxyphosphoryl-propan-2-one (1.30 eq, 2689 mg, 14.0 mmol) in Methanol (60 mL) was added K₂CO₃ (2.00 eq, 2976 mg, 21.5 mmol). Then 1-diazo-1-dimethoxyphosphoryl-propan-2-one (1.30 eq, 2689 mg, 14.0 mmol) was dissolved in Methanol (60 mL) was added dropwise at 25° C. under N₂ atmosphere. The reaction mixture was stirred at 25° C. for 12 hours. LCMS (5-95AB/1.5 min): RT=0.568 min, 266.0=[M+H]⁺, ESI⁺ showed 83% of desired product. The reaction mixture was filtered through a pad of celite. The filter cake was washed with MeOH (100 mL). The filtrate was concentrated under reduced pressure to afford a residue. The residue was diluted with water (80 mL) and then extracted with ethyl acetate (100 mL*3). The combined organic layers were washed with brine, dried over Na₂SO₄, filtered and concentrated under reduced pressure to give a residue. The residue was purified by flash chromatography on silica gel eluting with PE/EtOAc (2:1) (TLC, PE:EtOAc=0:1, Rf=0.70) to afford (2S)-2-ethynyl-4-(p-tolylsulfonyl)morpholine (2.50 g, 8.38 mmol, 77.79% yield) as withe solid. LCMS: Rt: 0.570 min; [M+H]⁺=266.0; 88.9% purity at 220 nm.

Step 4: To a stirred solution of cyclopropanamine (1.20 eq, 0.32 mL, 4.52 mmol) in MeCN (20 mL) was added diethylamine (6.00 eq, 2.4 mL, 22.6 mmol) and 2-azido-1,3-dimethyl-4,5-dihydroimidazol-1-ium; hexafluorophosphate (1.00 eq, 1075 mg, 3.77 mmol). The reaction mixture was stirred at 30° C. for 1 hour under N₂ atmosphere. Then (2S)-2-ethynyl-4-(p-tolylsulfonyl)morpholine (1.00 eq, 1000 mg, 3.77 mmol), copper(II) sulfate pentahydrate (0.200 eq, 188 mg, 0.754 mmol) and (+)-Sodium L-ascorbate (0.500 eq, 373 mg, 1.88 mmol) aqueous solution was added. The reaction mixture was stirred at 70° C. for 15 hours. LCMS (5-95AB/1.5 min): RT=0.851 min, 349.1=[M+H]⁺, ESI⁺ showed 93.3% of desired product. The reaction mixture was filtered through a filter. The filter cake was washed with MeCN (80 mL). The filtrate was concentrated under reduced pressure to afford a residue. The residue was diluted with water (80 mL) and then extracted with ethyl acetate (100 mL*3). The combined organic layers were washed with brine, dried over Na₂SO₄, filtered and concentrated under reduced pressure to give a residue. The residue was purified by flash chromatography on silica gel eluting with PE/EtOAc (1:1) (TLC, PE:EtOAc=0:1, R_{f}=0.35) to afford (2R)-2-(1-cyclopropyltriazol-4-yl)-4-(p-tolylsulfonyl)morpholine (1.17 g, 3.36 mmol, 89.10% yield) as a light-yellow solid, which LCMS (5-95AB/1.5 min): RT=0.858 min, 349.1=[M+H]⁺, ESI⁺ showed 99.1% of desired product. LCMS: Rt: 0.858 min; [M+H]⁺=349.1; 99.1% purity at 220 nm.

Step 5: To the mixture of (2R)-2-(1-cyclopropyltriazol-4-yl)-4-(p-tolylsulfonyl)morpholine (1.00 eq, 500 mg, 1.44 mmol) in Methanol (50 mL) was added Mg (12.0 eq, 413 mg, 17.2 mmol) (powder) and Mg (12.0 eq, 413 mg, 17.2 mmol) (chips) at 25° C. and the reaction mixture was stirred for 12 hours at 80° C. under N₂ atmosphere. LCMS (5-95CD/1.5 min): RT=0.234 min, 195.1=[M+H]⁺, ESI⁺ showed 50.8% of desired product and RT=0.870 min, 349.1=[M+H]⁺, ESI⁺ showed 47.7% of starting material. Then Mg (12.0 eq, 413 mg, 17.2 mmol) (powder) and Mg (12.0 eq, 413 mg, 17.2 mmol) (chips) was added and the mixture was stirred for 12 hours at 80° C. LCMS (5-95CD/1.5 min): RT=0.292 min, 195.1=[M+H]⁺, ESI⁺ showed 74.3% of desired product and RT=0.885 min, 349.1=[M+H]⁺, ESI⁺ showed 20.8% of starting material. The reaction mixture was filtered through a pad of celite. The filter cake was washed with MeOH (100 mL). The filtrate was concentrated under reduced pressure to afford crude product (2R)-2-(1-cyclopropyltriazol-4-yl)morpholine;4-methylbenzenesulfonic acid (770 mg, 1.03 mmol, 71.75% yield) as white solid. The crude product was used to next step without further purification.

Step 6: To a solution of 2-chloro-4-(2,4-difluorophenyl)-6,7-dimethyl-pteridine (1.00 eq, 50 mg, 0.137 mmol) and DIEA (4.00 eq, 0.091 mL, 0.548 mmol) in DMSO (2 mL) was added 2-(1-cyclopropyltriazol-4-yl)morpholine;4-methylbenzenesulfonic acid (1.20 eq, 60 mg, 0.164 mmol) at 25° C. Then the reaction mixture was stirred at 100° C. for 20 min. LCMS (5-95AB/1.5 min): RT=0.933 min, 465.2=[M+H]⁺, ESI⁺ showed 48.8% of desired product. The reaction was diluted with water (10 mL) and then extracted with ethyl acetate (15 mL*3). The combined organic layers were dried over Na₂SO₄, filtered and concentrated under reduced pressure to give a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 39%-69%; Detector, UV 254 nm. RT: [7 min]) to afford 2-(1-cyclopropyltriazol-4-yl)-4-[4-(2,4-difluorophenyl)-6,7-dimethyl-pteridin-2-yl]morpholine (32 mg, 0.0680 mmol, 49.68% yield) as a light-yellow solid. SFC (Column: Chiralpak IG-3 50×4.6 mm I.D., 3 um Mobile phase: Phase A for CO₂, and Phase B for MeOH+ACN (0.05% DEA); Gradient elution: 40% MeOH+ACN (0.05% DEA) in CO₂ Flow rate: 3 mL/min; Detector: PDA; Column Temp: 35° C.; Back Pressure: 100 Bar) showed two peaks and ratio was 25.9: 74.1. 2.2 mg product was delivered and 29.4 mg was kept in hand. I-1147, LCMS: Rt: 0.920 min; [M+H]⁺=465.2; 100% purity at 220 nm. ¹H NMR (400 MHz, CDCl3) δ ppm 1.14-1.22 (m, 2H) 1.23-1.28 (m, 2H) 2.60 (s, 3H) 2.72 (s, 3H) 3.33-3.43 (m, 2H) 3.78 (tt, J=7.37, 3.82 Hz, 1H) 3.86 (td, J=11.58, 2.75 Hz, 1H) 4.13 (dd, J=11.55, 1.90 Hz, 1H) 4.82 (dd, J=10.45, 2.75 Hz, 1H) 4.91 (br d, J=13.20 Hz, 1H) 5.21 (br d, J=12.84 Hz, 1H) 6.97 (td, J=9.48, 2.32 Hz, 1H) 7.04 (td, J=8.25, 1.83 Hz, 1H) 7.63 (s, 1H) 7.67-7.77 (m, 1H).

### Synthesis of Compounds 1-1155, 1-1249 and I-1244

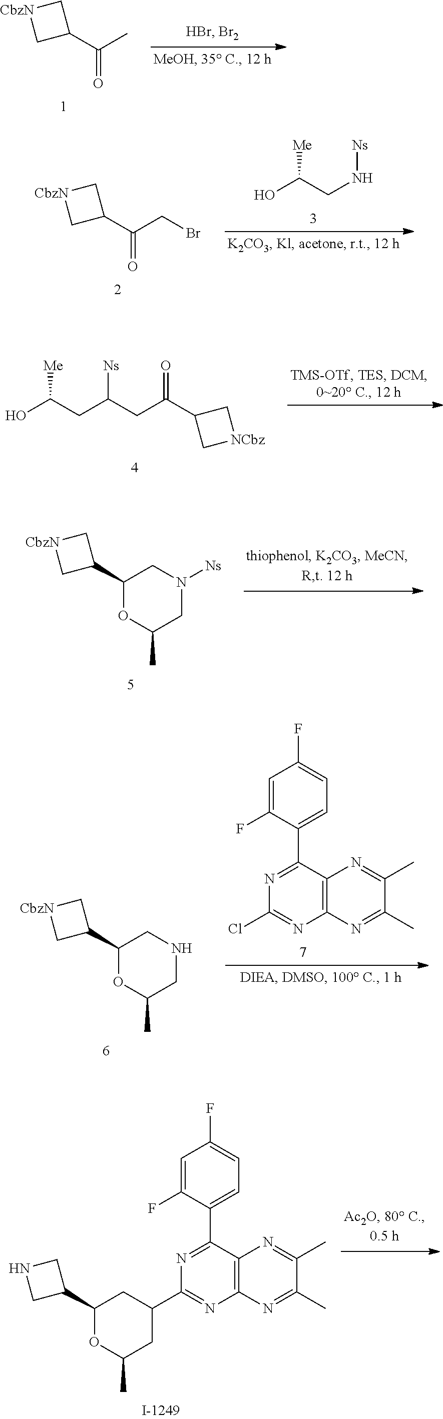

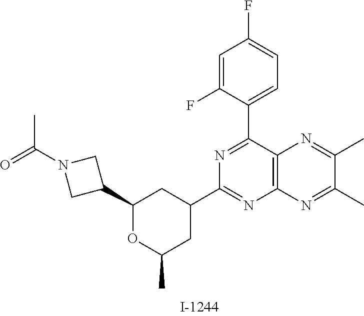 [SMILES: CC(=O)N1CC([C@H]2CC(c3nc(-c4ccc(F)cc4F)c4nc(C)c(C)nc4n3)C[C@@H](C)O2)C1]

Step 1: A mixture of benzyl 3-acetylazetidine-1-carboxylate (1.00 eq, 1000 mg, 4.29 mmol), HBr (0.100 eq, 86 mg, 0.429 mmol) in Methanol (10 mL) was added bromine (1.00 eq, 0.22 mL, 4.29 mmol) at 0° C., then the mixture was stirred at 35° C. for 12 hr. LCMS showed 14% starting material was remained and 33% desired MS (311.7 [M+1]⁺, ESI pos) was found. The pH was adjusted to 7 with NaHCO₃, and the mixture was extracted with ethyl acetate (50 mL×3) and concentrated to give a residue. The solution was purified by column on silica (PE:EA=2:1) and concentrated under reduced pressure to give a yellow oil. Benzyl 3-(2-bromoacetyl)azetidine-1-carboxylate (327 mg, 1.05 mmol, 24.43% yield) was obtained as a yellow oil and used to the next step. ¹H NMR (400 MHz, CDCl3) δ=7.40-7.30 (m, 5H), 5.11 (s, 2H), 4.19 (d, J=7.5 Hz, 4H), 3.94-3.82 (m, 3H).

Step 2: A mixture of benzyl 3-(2-bromoacetyl)azetidine-1-carboxylate (1.00 eq, 300 mg, 0.961 mmol), N-[(2R)-2-hydroxypropyl]-4-nitro-benzenesulfonamide (1.50 eq, 375 mg, 1.44 mmol), K₂CO₃ (3.00 eq, 398 mg, 2.88 mmol) and KI (1.00 eq, 160 mg, 0.961 mmol) in Acetone (8 mL) was stirred at 20° C. for 12 hr. LCMS showed the starting material was consumed completely and 71% desired MS (492.1.0 [M+1]⁺, ESI pos) was found. The mixture was poured into water (50 mL) and extracted by ethyl acetate (3*50 mL), the organic phase was dried over Na₂SO₄, filtered and concentrated in vacuum. The solution was purified by column on silica (PE:EA=1: 1) and concentrated under reduced pressure to give a yellow oil. Benzyl 3-[2-[(4-nitrophenyl)sulfonyl-[rac-(2R)-2-hydroxypropyl]amino]acetyl]azetidine-1-carboxylate (270 mg, 0.522 mmol, 54.30% yield) was obtained as a yellow oil. ¹H NMR (400 MHz, CDCl3) δ=8.41 (d, J=8.8 Hz, 2H), 7.96 (d, J=8.8 Hz, 2H), 7.44-7.27 (m, 5H), 5.09 (s, 2H), 4.21-4.15 (m, 1H), 4.06-3.92 (m, 4H), 3.71 (br d, J=11.4 Hz, 1H), 3.57 (br d, J=10.6 Hz, 1H), 3.45 (br s, 1H), 2.75-2.63 (m, 1H), 2.32-2.23 (m, 1H), 2.12 (br t, J=11.2 Hz, 1H), 1.13 (d, J=6.2 Hz, 3H).

Step 3: A mixture of benzyl 3-[2-[[(2R)-2-hydroxypropyl]-(4-nitrophenyl)sulfonyl-amino]acetyl]azetidine-1-carboxylate (1.00 eq, 270 mg, 0.549 mmol) in DCM (10 mL) was added TES (5.00 eq, 630 mg, 2.75 mmol) at 0° C., TMSOTf (5.00 eq, 610 mg, 2.75 mmol) was added at 0° C., then the mixture was stirred at 0° C. for 2 hr. LCMS showed the starting material was consumed completely and 88% desired MS (476.0 [M+1]⁺, ESI pos) was found. The combined mixture was poured into saturated aq NaHCO₃ (20 mL) and extracted by ethyl acetate (3*20 mL), the organic phase was dried over Na₂SO₄, filtered and concentrated in vacuum. The solution was purified by flash chromatography (0.7 g SiO₂ cartridge, PE:EA=1: 1, detection at 254 nm) and concentrated under reduced pressure to give a yellow oil. Benzyl 3-[(2S,6R)-6-methyl-4-(4-nitrophenyl)sulfonyl-morpholin-2-yl]azetidine-1-carboxylate (260 mg, 0.492 mmol, 89.58% yield) was obtained as a yellow oil. ¹H NMR (400 MHz, CDCl3) δ=8.46-8.38 (m, 2H), 7.97-7.90 (m, 2H), 7.40-7.29 (m, 5H), 5.09 (s, 2H), 4.08-4.02 (m, 1H), 4.02-3.96 (m, 1H), 3.91 (dd, J=5.6, 8.7 Hz, 1H), 3.86 (dd, J=5.7, 8.7 Hz, 1H), 3.78-3.70 (m, 2H), 3.65 (td, J=1.9, 11.3 Hz, 1H), 3.58 (br d, J=11.0 Hz, 1H), 2.60-2.49 (m, 1H), 2.05-1.91 (m, 2H), 1.16 (d, J=6.3 Hz, 3H).

Step 4: A mixture of benzyl 3-[(2S,6R)-6-methyl-4-(4-nitrophenyl)sulfonyl-morpholin-2-yl]azetidine-1-carboxylate (1.00 eq, 260 mg, 0.547 mmol) and K₂CO₃ (5.00 eq, 378 mg, 2.73 mmol) in MeCN (5 mL) was added thiophenol (5.00 eq, 301 mg, 2.73 mmol), then the mixture was stirred at 15° C. for 12 hr. LCMS showed the starting material was consumed completely and 50% desired MS (290.9 [M+1]⁺, ESI pos) was found. The mixture was quenched with saturated aq NaHCO₃ (30 mL) and extracted with EtOAc (3*50 mL), the organic phase was concentrated to give a residue. The combined aqueous phase was poured into NaCl aqueous (50 mL) and standing overnight and poured into recycling bucket. The solution was purified by prep-HPLC (NH₄HCO₃, column: Waters Xbridge 150*25 mm * 5 um; mobile phase: [water (NH₄HCO₃)-ACN]; B %: 18%-48%, 10 min), the purified solution was lyophilized to give a brown oil. benzyl 3-[(2S,6R)-6-methylmorpholin-2-yl]azetidine-1-carboxylate (74 mg, 0.255 mmol, 46.61% yield) was obtained as a brown oil. ¹H NMR (400 MHz, CDCl3) δ=7.40-7.27 (m, 5H), 5.10 (s, 2H), 4.10-3.96 (m, 3H), 3.81 (dd, J=5.8, 8.4 Hz, 1H), 3.64-3.52 (m, 2H), 2.90-2.75 (m, 2H), 2.63-2.49 (m, 1H), 2.47-2.30 (m, 2H), 1.12 (d, J=6.3 Hz, 3H).

Step 5: To a mixture of benzyl 3-[(2S,6R)-6-methylmorpholin-2-yl]azetidine-1-carboxylate (1.00 eq, 10 mg, 0.0344 mmol) in DMSO (1 mL) was added 2-chloro-4-(2,4-difluorophenyl)-6,7-dimethyl-pteridine (1.00 eq, 11 mg, 0.0344 mmol) and DIPEA (3.00 eq, 0.018 mL, 0.103 mmol), then the mixture was stirred at 100° C. for 1 hr. LCMS showed the starting material was consumed completely and 51% desired MS (561.2 [M+1]⁺, ESI pos) was found. The reaction mixture was cooled to room temperature. The mixture was purified by prep-HPLC (FA, column: Phenomenex luna C18 150*25 mm* 10 um; mobile phase: [water (FA)-ACN]; B %: 65%-85%, 10 min), the purified solution was lyophilized to give a yellow solid. I-1155 (3.4 mg, 0.00598 mmol, 17.36% yield) was obtained as a yellow solid. LCMS Rt: 1.047 min, m/z: 561.3 [M+H]⁺. 97.441% purity at 214 nm. ¹H NMR (400 MHz, CDCl3) δ=7.76-7.66 (m, 1H), 7.43-7.27 (m, 5H), 7.09-7.01 (m, 1H), 6.98 (dt, J=2.3, 9.4 Hz, 1H), 5.11 (s, 2H), 4.91 (br d, J=7.0 Hz, 1H), 4.84 (br d, J=12.9 Hz, 1H), 4.15-4.06 (m, 3H), 4.02-3.90 (m, 1H), 3.76-3.64 (m, 2H), 2.78-2.65 (m, 6H), 2.59 (s, 3H), 1.28 (d, J=6.1 Hz, 3H). HPLC: Rt: 2.683 min, 99.297% purity at 214 nm. Chiral Purity: Rt: 1.988 min, 100.00%

Step 6: To a mixture of 1-[3-[(2S,6R)-4-[4-(2,4-difluorophenyl)-6,7-dimethyl-pteridin-2-yl]-6-methyl-morpholin-2-yl]azetidin-1-yl]-2,2,2-trifluoro-ethanone (1.00 eq, 10 mg, 0.0191 mmol) in Ethanol (0.5000 mL) and Water (0.5000 mL) was added K₂CO₃ (3.00 eq, 7.9 mg, 0.0574 mmol), then the mixture was stirred at 40° C. for 1 hr to give a yellow solution. LCMS showed the starting material was consumed completely and 59% desired MS (427.1 [M+1]⁺, ESI pos) was found. The reaction mixture was cooled to room temperature. The solution was purified by prep-HPLC (column: Shim-pack C18 150* 25* 10 um; mobile phase: [water (FA)-ACN]; B %: 20%-50%, 10 min), the purified solution was lyophilized to give a yellow solid. I-1249 (3.1 mg, 0.00594 mmol, 31.05% yield) was obtained as a yellow solid. (90% purity, mixed with impurity and ACN). I-1249: LC-MS: Rt: 0.652 min, m/z: 427.0 [M+H]⁺. 96.118% purity at 214 nm. ¹H NMR (400 MHz, DMSO+D2O) δ=8.62 (s, 1H), 7.76-7.67 (m, 1H), 7.06 (dt, J=2.1, 8.2 Hz, 1H), 6.98 (dt, J=2.3, 9.5 Hz, 1H), 5.02-4.72 (m, 2H), 4.21-3.93 (m, 4H), 3.84-3.66 (m, 2H), 3.13-2.95 (m, 2H), 2.74 (br d, J=2.6 Hz, 1H), 2.72 (s, 3H), 2.60 (s, 3H), 1.35-1.27 (m, 3H). HPLC: Rt: 1.647 min, 89.991% purity at 214 nm

Step 7: To a mixture of (2S,6R)-2-(azetidin-3-yl)-4-[4-(2,4-difluorophenyl)-6,7-dimethyl-pteridin-2-yl]-6-methyl-morpholine (1.00 eq, 13 mg, 0.0305 mmol) in acetic anhydride (348 eq, 1.0 mL, 10.6 mmol) was stirred at 80° C. for 0.5 hr to give a red solution. LCMS showed the starting material was consumed completely and 56.6% desired MS (469.1 [M+1]⁺, ESI pos) was found. The reaction mixture was cooled to room temperature. The solution was purified by prep-HPLC (FA, column: Phenomenex luna C18 150*25 mm*10 um; mobile phase: [water (FA)-ACN]; B %: 43%-73%, 2 min), the purified solution was lyophilized to give a brown oil. LCMS showed 67.4% desired MS (469.0 [M+1]⁺, ESI pos) was found. The solution was re-purified by prep-TLC (DCM:MeOH=30:1, Rf=0.3), the purified solution was concentrated to give a yellow oil. The purified solution was lyophilized to give a yellow solid. 1-1244 (2.7 mg, 0.00526 mmol, 17.27% yield) was obtained as a yellow solid. (90% purity, mixed with impurity and ACN). 1-1244: LC-MS: Rt: 0.768 min, m/z: 469.0 [M+H]⁺. 95.999% purity at 214 nm. ¹H NMR (400 MHz, CDCl3) δ=7.75-7.66 (m, 1H), 7.08-7.02 (m, 1H), 6.97 (dt, J=2.3, 9.5 Hz, 1H), 4.99-4.79 (m, 2H), 4.09 (br s, 4H), 3.76-3.64 (m, 2H), 2.78-2.65 (m, 5H), 2.59 (s, 3H), 1.87 (s, 3H), 1.28 (d, J=6.3 Hz, 6H). HPLC: Rt: 2.136 min, 94.721% purity at 214 nm.

### Synthesis of Compound I-1160

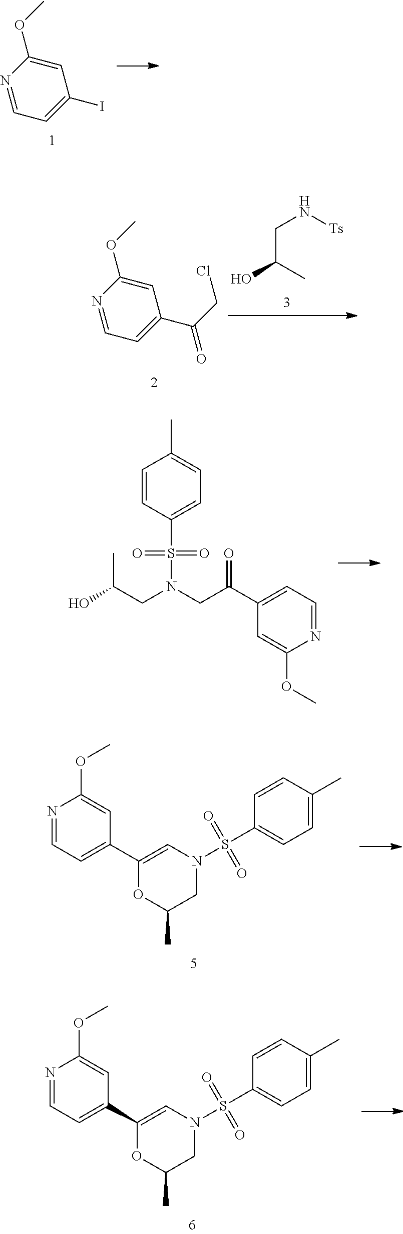

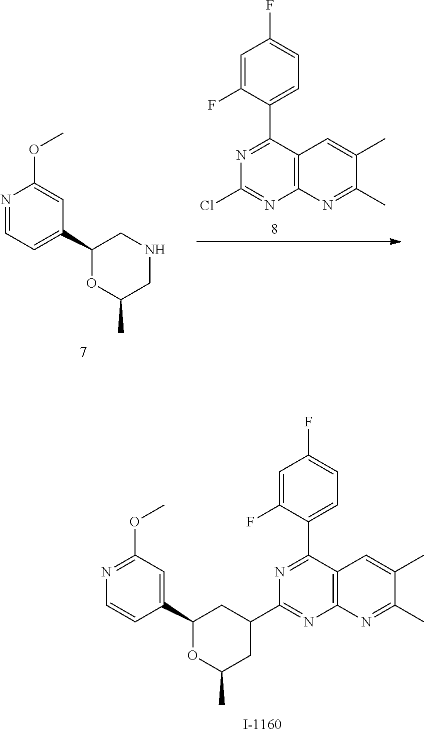 [SMILES: COc1cc([C@H]2CNC[C@@H](C)O2)ccn1 ; Cc1cc2c(-c3ccc(F)cc3F)nc(Cl)nc2nc1C ; COc1cc([C@H]2CC(c3nc(-c4ccc(F)cc4F)c4cc(C)c(C)nc4n3)C[C@@H](C)O2)ccn1]

Step 1: To a solution of 4-iodo-2-methoxy-pyridine (1.00 eq, 1000 mg, 4.25 mmol) in THF (15 mL) was isopropylmagnesium chloride lithium chloride complex (1.20 eq, 3.9 mL, 5.11 mmol) at −78° C. and stirred for 30 min, then 2-chloro-N-methoxy-N-methylacetamide (1.10 eq, 644 mg, 4.68 mmol) was added to the mixture and stirred at 0° C. for 1 hr. LCMS showed raw material was consumed completely and the major peak with desired MS (185.7 [M+H]⁺; ESI⁺) was detected. The reaction was quenched by NH₄Cl (aq. 20 mL) and then extracted with EtOAc (20 mL*2) and the organics washed with 10 mL saturated brine solution. The organics were then separated and dried (Na₂SO₄) before concentration to dryness. The crude was then purified by silica gel column (PE/EA=1/1, Rf=0.6) to give 2-chloro-1-(2-methoxy-3,4-dihydropyridin-4-yl)ethanone (460 mg, 2.45 mmol, 57.62% yield) as a light-yellow solid. 1H NMR (400 MHz, CDCl3) δ=8.38-8.33 (m, 1H), 7.32-7.29 (m, 1H), 7.20-7.16 (m, 1H), 4.67-4.64 (m, 2H), 4.02-3.98 (in, 3H).

Step 2: To a solution of N-[(2R)-2-hydroxypropyl]-4-methyl-benzenesulfonamide (1.50 eq, 4077 mg, 17.8 mmol) and 2-chloro-1-(2-methoxy-4-pyridyl)ethanone (1.00 eq, 2200 mg, 11.9 mmol) in Acetone (20 mL) was added K₂CO₃ (3.00 eq, 4914 mg, 35.6 mmol) and KI (1.00 eq, 1968 mg, 11.9 mmol) and the mixture was stirred for 2 h at 25° C. LCMS showed raw material was consumed and the major peak showed desired MS (378.8[M+H]+,ESI+; LC-RT: 0.911 min). The reaction was poured into water (30 mL) and then extracted with EtOAc (20 mL*3) and the organics washed with 20 mL saturated brine solution. The organics were then separated and dried (Na₂SO₄) before concentration to dryness. The crude was then purified by silica gel column (PE/EA=3/1, R_{f}=0.5) to give N-[(2R)-2-hydroxypropyl]-N-[2-(2-methoxy-4-pyridyl)-2-oxo-ethyl]-4-methyl-benzenesulfonamide (1300 mg, 3.44 mmol, 28.98% yield) as a light-yellow solid. 1H NMR (400 MHz, CDCl3) δ=8.16 (d, J=5.4 Hz, 1H), 7.63-7.57 (m, 2H), 7.35-7.31 (m, 2H), 7.08-7.04 (m, 1H), 6.95-6.91 (m, 1H), 4.42-4.34 (m, 1H), 4.01-3.96 (m, 2H), 3.95-3.92 (m, 3H), 3.76-3.65 (m, 3H), 2.45-2.43 (m, 3H), 1.25-1.22 (m, 3H).

Step 3: To a solution of N-[(2R)-2-hydroxypropyl]-N-[2-(2-methoxy-4-pyridyl)-2-oxo-ethyl]-4-methyl-benzenesulfonamide (1.00 eq, 1400 mg, 3.70 mmol) in DCM (100 mL) was added triethylsilane (5.00 eq, 2146 mg, 18.5 mmol) and trimethylsilyl trifluoromethanesulfonate (5.00 eq, 3.3 mL, 18.5 mmol) at 0° C. and the mixture was stirred for 12 h at 25° C. LCMS showed the raw material was consumed completely and the major peak showed desired MS (360.8 [M+H]⁺; ESI⁺, LC-RT=0.940 min). The reaction was wash with water (100 mL) and then the organics were then separated and dried (Na₂SO₄) before concentration to dryness and give (2R)-6-(2-methoxy-4-pyridyl)-2-methyl-4-(p-tolylsulfonyl)-2,3-dihydro-1,4-oxazine (1250 mg, 3.47 mmol, 93.75% yield) as a light-yellow oil.

Step 4: To a solution of (2R)-6-(2-methoxy-4-pyridyl)-2-methyl-4-(p-tolylsulfonyl)-2,3-dihydro-1,4-oxazine (1.00 eq, 300 mg, 0.832 mmol) in Methanol (10 mL) was added Pd/C (3.40 eq, 300 mg, 2.83 mmol) under N₂ atmosphere. The mixture was purged with H₂ 3 times, then the mixture was stirred at 30° C. under H₂ (15 psi) for 12 h. LCMS showed raw material was consumed completely and the major peak showed desired MS (362.8 [M+H]+; ESI+, LC-RT=0.970 min). The reaction was filtered and the filtrate was concentrated in vacuum to give the residue and then purified by silica gel column (PE/EA=1/3, R_{f}=0.6) to give (2S,6R)-2-(2-methoxy-4-pyridyl)-6-methyl-4-(p-tolylsulfonyl)morpholine (200 mg, 0.552 mmol, 66.30% yield) as white solid.

Step 5: To a solution of (2S,6R)-2-(2-methoxy-4-pyridyl)-6-methyl-4-(p-tolylsulfonyl)morpholine (1.00 eq, 150 mg, 0.414 mmol) in Methanol (10 mL) was added Mg (powder, 10.6 eq, 105 mg, 4.38 mmol) and Mg (chips, 10.6 eq, 105 mg, 4.38 mmol) at 25° C. and then the mixture was stirred for 12 h at 80° C. under N₂. White suspension formed. LCMS showed raw material was consumed and the major peak with desired MS (208.8 [M+H]+; ESI+, LC-RT: 0.251 min). The reaction was added MeOH (20 mL) and filtered, the filter cake was washed with MeOH (20 mL*2). The filtrate was concentrated in vacuum to give crude (2S,6R)-2-(2-methoxy-4-pyridyl)-6-methyl-morpholine (130 mg, 0.624 mmol, 150.83% yield) as white solid.

Step 6: To a solution of (2S,6R)-2-(2-methoxy-4-pyridyl)-6-methyl-morpholine (1.00 eq, 100 mg, 0.480 mmol), 2-chloro-4-(2,4-difluorophenyl)-6,7-dimethyl-pyrido[2,3-d]pyrimidine (1.00 eq, 171 mg, 0.480 mmol) in DMSO (3 mL) was added DIEA (4.00 eq, 248 mg, 1.92 mmol) and then the mixture was stirred for 20 min at 100° C. LCMS showed raw material was consumed and the major peak showed desired MS (478.2 [M+H]+; ESI+, LC-RT: 0.888 min). The reaction mixture was poured into water (15 mL) and then extracted with EtOAc (10 mL*2) and the organics washed with 5 mL saturated brine solution. The organics were then separated and dried (Na₂SO₄) before concentration to dryness. The crude mixture was then purified by prep-HPLC (Phenomenex Luna C18 150*25 mm*10 um, water (FA)-ACN) to give (2S,6R)-4-[4-(2,4-difluorophenyl)-6,7-dimethyl-pyrido[2,3-d]pyrimidin-2-yl]-2-(2-methoxy-4-pyridyl)-6-methyl-morpholine (4.9 mg, 0.0101 mmol, 2.11% yield) as yellow solid which was H NMR. LCMS: (M+H)+=478.2; purity=98.55% (UV=220 nm). Retention time=0.910 min. 1H NMR (400 MHz, CDCl3) δ=8.18-8.14 (m, 1H), 7.60-7.50 (m, 2H), 7.14-6.97 (m, 3H), 6.91-6.86 (m, 1H), 5.20-4.96 (m, 2H), 4.63 (s, 1H), 3.98-3.93 (m, 3H), 3.90-3.81 (m, 1H), 2.94-2.75 (m, 2H), 2.72-2.65 (m, 3H), 2.38-2.31 (m, 3H), 1.39-1.34 (m, 3H).

### Synthesis of 1-1170

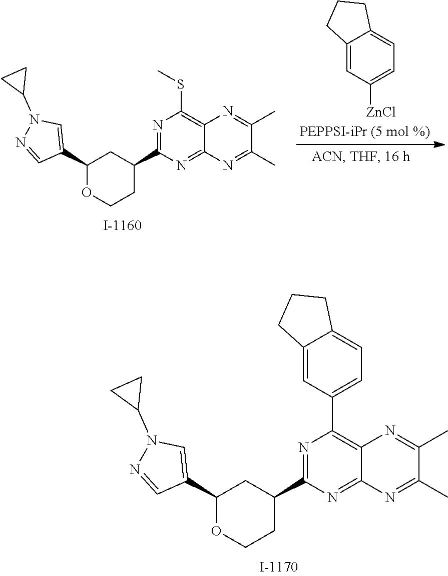

A flame-dried microwave vial under N₂ was charged with PEPPSI™-SIPr (0.05 eq, 4.3 mg, 0.006 mmol), chloro(indan-5-yl)zinc (4.00 eq, 3.9 mL, 0.50 mmol) and the mixture was then cooled to 0° C. MeCN (0.42 mL) was added followed by 2-[(2R,4S)-2-(1-cyclopropylpyrazol-4-yl)tetrahydropyran-4-yl]-6,7-dimethyl-4-methylsulfanyl-pteridine (1.00 eq, 50 mg, 0.13 mmol) and the resulting mixture was stirred at room temperature for 12 h. The mixture was filtered through a pad of Celite, washed with DCM (2×10 mL), and concentrated under reduced pressure. The crude material was purified by normal phase flash chromatography on a 10 g Biotage column using a gradient from 50% to 100% EtOAc in Hexanes followed by a 0 to 20% MeOH in DCM wash. The desired fractions were evaporated under reduced pressure to afford the desired analog as a mixture of diastereoisomers. The desired cis diastereoisomer was isolated by Prep-HPLC purification (Gemini® 5 um NX-C18 110 Å, 100×30 mm) using aqueous 10 mM Ammonium Bicarbonate (pH=10.0) and ACN (65-85%). 4-indan-5-yl-6,7-dimethyl-2-[(2R,4S)-2-(1-cyclopropylpyrazol-4-yl) tetrahydropyran-4-yl]pteridine (22 mg, 0.05 mmol, 37% yield). ESI-MS (m/z+): 467.3 [M+H]⁺. ¹H NMR (CHCl₃-d, 400 MHz): δ_{H} 8.16-8.20 (2H, m), 7.49 (2H, s), 7.39 (1H, d, J=7.8 Hz), 4.54 (1H, dd, J=11.4, 2.1 Hz), 4.25 (1H, d, J=11.5 Hz), 3.77-3.84 (1H, m), 3.50-3.56 (1H, m), 3.01 (4H, dt, J=11.6, 7.4 Hz),2.79 (6H, dd, J=20.2, 0.0 Hz), 2.43 (1H, d, J=13.4 Hz), 2.10-2.26 (5H, m), 1.05-1.09 (2H, m), 0.93-0.98 (2H, m).

### Synthesis of I-1182

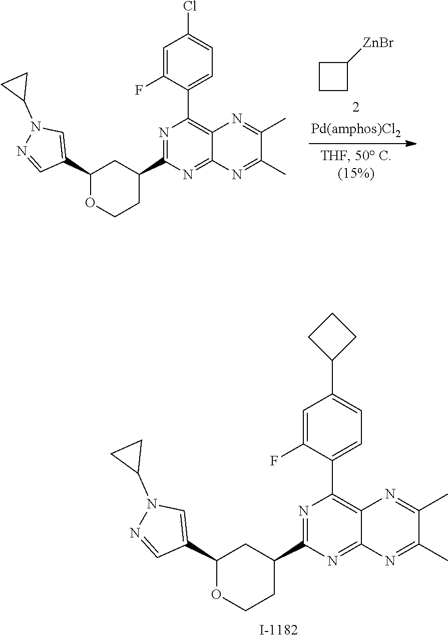

A flame-dried glass vial equipped with a Teflon-coated magnetic stirring bar was charged with 4-(4-chloro-2-fluoro-phenyl)-2-[(2R,4S)-2-(1-cyclopropylpyrazol-4-yl)tetrahydropyran-4-yl]-6,7-dimethyl-pteridine (prepared via Method 37) (1.00 equiv, 25 mg, 0.052 mmol) and Pd(amphos)Cl₂ (0.25 eq, 9.2 mg, 0.013 mmol), under Ar (g). Degassed, anhydrous THF (1.0 mL) was added, and the vial was sealed and purged under Ar (g). A 0.37 M solution of bromo(cyclobutyl)zinc (1.5 equiv, 0.21 mL, 0.078 mmol) in THF was added and the reaction mixture was stirred at 50° C. for 6 h. The reaction mixture was cooled to RT, diluted with EtOAc and quenched with sat. NH₄Cl (aq). The layers were separated, and the organic layer was washed with sat. NH₄Cl (aq) and brine. The organic layer was collected, dried over Na₂SO₄, filtered and evaporated to dryness under reduced pressure. The crude residual material was purified by flash chromatography (Teledyne RediSep GOLD column, 12 g SiO₂) using an elution gradient of 0% to 10% MeOH in DCM to yield 22 mg of crude product. This was further purified by prep HPLC (Gemini® 5 um NX-C18 110 Å, 100×30 mm column) using an elution gradient of MeCN in 10 mM aqueous ammonium formate pH 3.8 (50-70%) to afford 4-(4-cyclobutyl-2-fluoro-phenyl)-2-[(2R,4S)-2-(1-cyclopropylpyrazol-4-yl)tetrahydropyran-4-yl]-6,7-dimethyl-pteridine (3.5 mg, 0.007 mmol, 13% yield) as a light-yellow solid. ESI-MS (m/z+): 499.3 [M+1]⁺, LC-RT: 1.80 min. ¹H NMR (DMSO-d₆, 400 MHz): δ_{H} 7.73 (1H, s), 7.65 (1H, t, J=7.6 Hz), 7.39 (1H, s), 7.28 (1H, s), 7.26 (1H, d, J=2.9 Hz), 4.51 (1H, d, J=11.2 Hz), 4.10 (1H, dd, J=11.3, 4.3 Hz), 3.63-3.74 (4H, m), 3.41-3.50 (1H, m), 2.77 (3H, s), 2.65 (3H, s), 2.28-2.37 (2H, m), 2.14-2.24 (2H, m), 1.87-2.04 (6H, m), 0.98-1.00 (2H, m), 0.89-0.94 (2H, m).

### Synthesis of I-1187 (Same general method for I-1192 and I-1197)

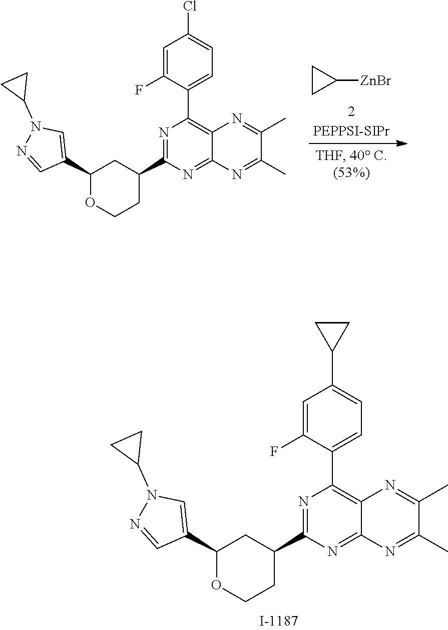

A flame-dried glass vial equipped with a Teflon-coated magnetic stirring bar was charged with 4-(4-chloro-2-fluoro-phenyl)-2-[(2R,4S)-2-(1-cyclopropylpyrazol-4-yl)tetrahydropyran-4-yl]-6,7-dimethyl-pteridine (prepared via Method 37) (1.00 equiv, 50 mg, 0.10 mmol) and PEPPSI-SIPr (0.25 equiv, 18 mg, 0.026 mmol), under Ar (g). Degassed, anhydrous THF (1.5 mL) was added, and the vial was sealed and purged under Ar (g). The reaction mixture was heated to 40° C. with stirring and bromo(cyclopropyl)zinc 2 (0.39 M in THF) (3.0 equiv, 0.80 mL, 0.31 mmol) was added dropwise over 1 h. The reaction mixture was stirred at 40° C. for an additional 1 h. The reaction mixture was cooled to RT, diluted with EtOAc and quenched with sat. NH₄Cl (aq). The layers were separated, and the organic layer was washed with sat. NH₄Cl (aq) and brine. The organic layer was collected, dried over Na₂SO₄, filtered and evaporated to dryness under reduced pressure. The crude residual material was purified by silica gel flash chromatography (Teledyne RediSep GOLD column, 12 g SiO₂) using an elution gradient of 0% to 10% MeOH in DCM to yield 44 mg of crude product. This was further purified by prep HPLC (Gemini® 5 um NX-C18 110 Å, 100×30 mm column) using an elution gradient of MeCN in 10 mM aqueous ammonium formate pH 3.8 (40-60%) to afford 2-((2R,4S)-2-(1-cyclopropyl-1H-pyrazol-4-yl)tetrahydro-2H-pyran-4-yl)-4-(4-cyclopropyl-2-fluorophenyl)-6,7-dimethylpteridine (27 mg, 0.056 mmol, 53% yield) as a yellow solid. ESI-MS (m/z+): 485.2 [M+1]⁺, LC-RT: 1.64 min. ¹H NMR (DMSO-d₆, 400 MHz): δ_{H} 7.73 (1H, s), 7.59 (1H, t, J=7.7 Hz), 7.38 (1H, s), 7.13 (1H, dd, J=8.0, 1.6 Hz), 7.10 (1H, dd, J=11.6, 1.6 Hz), 4.51 (1H, d, J=11.2 Hz), 4.10 (1H, dd, J=11.3, 4.1 Hz), 3.61-3.72 (2H, m), 3.42-3.48 (1H, m), 2.77 (3H, s), 2.65 (3H, s), 2.30 (1H, d, J=13.4 Hz), 2.03-2.08 (2H, m), 1.87-1.99 (2H, m), 1.05-1.11 (2H, m), 0.96-1.01 (2H, m), 0.87-0.94 (2H, m), 0.81-0.84 (2H, m).

### Synthesis of I-1202

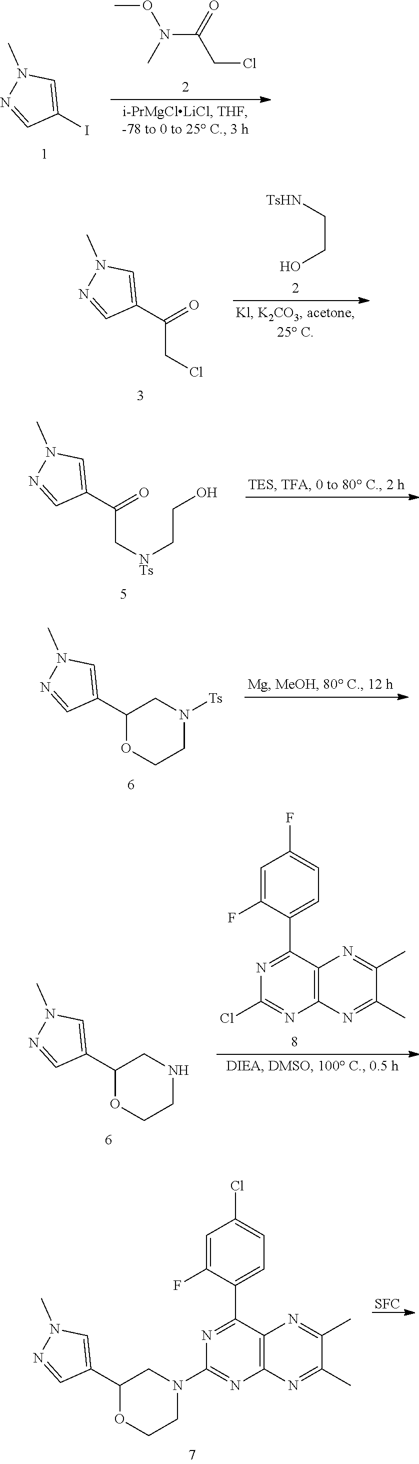

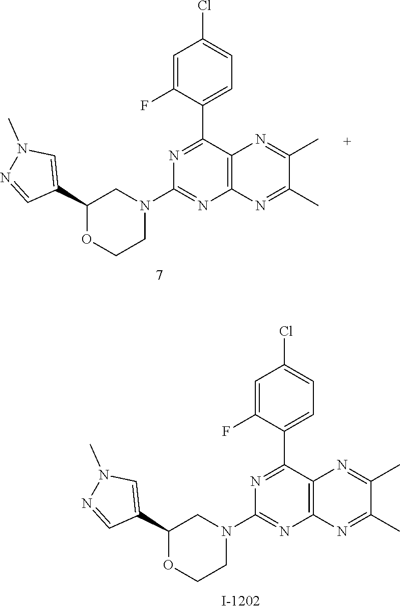 [SMILES: Cc1nc2nc(N3CCO[C@@H](c4cnn(C)c4)C3)nc(-c3ccc(Cl)cc3F)c2nc1C ; Cc1nc2nc(N3CCO[C@@H](c4cnn(C)c4)C3)nc(-c3ccc(Cl)cc3F)c2nc1C]

Step 1: To a solution of 4-iodo-1-methyl-pyrazole (1.00 eq, 5000 mg, 24.0 mmol) in THF (100 mL) was added Isopropylmagnesium chloride lithium chloride complex (1.3 M solution in THF) (1.20 eq, 22 mL, 28.8 mmol) at −78° C., the mixture was stirred at −78° C. for 0.5 h. Then added 2-chloro-N-methoxy-N-methylacetamide (3.00 eq, 9920 mg, 72.1 mmol) at −78° C. and the mixture was stirred at 0° C. for 1 h. LCMS showed starting material consumed and desired product (20%, Rt: 0.541 min; [M+H]⁺=159.1 at 220 nm) was formed. The reaction mixture was quenched with sat. NH₄Cl (200 mL), extracted with EtOAc (100 mL three times). The combined organic phase was washed by brine (50 mL), dried by Na₂SO₄, purified by flash column (PE to EtOAc condition, 70% to 100%) and concentrated to give 2-chloro-1-(1-methylpyrazol-4-yl)ethanone (1.50 g, 9.46 mmol, 39.35% yield) as off-white solid which 1H NMR. [M+H]⁺=159.1. Retention time=0.541 min. ¹H NMR (400 MHz, CDCl3) δ=7.99 (s, 1H), 7.96 (s, 1H), 4.40 (s, 2H), 3.96 (s, 3H).

Step 2: A mixture of 2-chloro-1-(1-methylpyrazol-4-yl)ethanone (1.00 eq, 500 mg, 3.15 mmol), N-(2-hydroxyethyl)-4-methyl-benzenesulfonamide (2.00 eq, 1357 mg, 6.31 mmol), KI (1.00 eq, 523 mg, 3.15 mmol) and K₂CO₃ (3.00 eq, 1307 mg, 9.46 mmol) in Acetone (10 mL) was stirred at 25° C. for 1 h. LCMS showed starting material consumed and desired product (39%, Rt: 0.489 min; [M+H]⁺=338.0 at 220 nm) was formed. The reaction mixture was poured into water (50 mL), extracted with EtOAc (30 mL three times). The combined organic phase was washed by brine (30 mL), dried by Na₂SO₄, purified by flash column(PE to EtOAc condition, 30% to 100%) and concentrated to give N-(2-hydroxyethyl)-4-methyl-N-[2-(1-methylpyrazol-4-yl)-2-oxo-ethyl]benzenesulfonamide (510 mg, 1.51 mmol, 47.95% yield) as off-white solid. [M+H]⁺=338.0. Retention time=0.489 min. ¹H NMR (400 MHz, CDCl3) δ=8.01 (s, 1H), 7.95 (s, 1H), 7.75 (d, J=8.1 Hz, 2H), 7.33 (d, J=8.3 Hz, 2H), 4.49 (s, 2H), 3.97 (s, 3H), 3.64 (br s, 2H), 3.37 (t, J=4.8 Hz, 2H), 2.44 (s, 3H).

Step 3: A solution of N-(2-hydroxyethyl)-4-methyl-N-[2-(1-methylpyrazol-4-yl)-2-oxo-ethyl]benzenesulfonamide (1.00 eq, 696 mg, 2.06 mmol) in TFA (95.0 eq, 15 mL, 196 mmol) was added triethylsilane (15.2 eq, 5.0 mL, 31.3 mmol) at 0° C. slowly and then stirred at 80° C. for 2 h. LCMS showed starting material consumed and desired product (99%, Rt: 0.549 min; [M+H]⁺=322.1 at 220 nm) is formed. The reaction mixture was quenched with sat. NaHCO₃ (100 mL)(adjusted to pH >8), extracted with EtOAc (50 mL three times). The combined organic phase was washed by brine (50 mL), dried by Na₂SO₄, purified by flash column (PE to EtOAc condition, 60% to 80%) and concentrated to give 2-(1-methylpyrazol-4-yl)-4-(p-tolylsulfonyl)morpholine (634 mg, 1.97 mmol, 95.63% yield) as a light-yellow oil which ¹H NMR: [M+H]⁺=322.1. Retention time=0.549 min. ¹H NMR (400 MHz, CDCl3) δ=7.64 (d, J=8.3 Hz, 2H), 7.41 (s, 1H), 7.36 (s, 1H), 7.34 (s, 1H), 7.33 (s, 1H), 4.61 (dd, J=2.7, 9.8 Hz, 1H), 4.01-3.94 (m, 1H), 3.87 (s, 3H), 3.79 (dt, J=2.7, 11.3 Hz, 1H), 3.67 (td, J=2.1, 11.5 Hz, 1H), 3.53 (dd, J=1.9, 11.4 Hz, 1H), 2.54 (dt, J=3.3, 11.2 Hz, 1H), 2.45 (s, 3H), 2.40 (dd, J=9.9, 11.4 Hz, 1H).

Step 4: To the mixture of 2-(1-methylpyrazol-4-yl)-4-(p-tolylsulfonyl)morpholine (1.00 eq, 700 mg, 2.18 mmol) in Methanol (70 mL) was added Mg (12.0 eq, 627 mg, 26.1 mmol)(as powder) and Mg (12.0 eq, 627 mg, 26.1 mmol) (as chips) at 25° C. and the reaction mixture was stirred for 12 hours at 80° C. under N₂ atmosphere. LCMS showed starting material consumed and desired product (87%, Rt: 0.428 min; [M+H]⁺=168.1 at 220 nm) was formed. The reaction mixture was filtered and the filtrate was concentrated under reduced pressure to give 4-methylbenzenesulfonic acid; 2-(1-methylpyrazol-4-yl)morpholine (640 mg, 1.89 mmol, 86.57% yield) as yellow solid. [M+H]⁺=168.1. Retention time=0.428 min. ¹H NMR (400 MHz, DMSO-d6) δ=7.59 (s, 1H), 7.44-7.35 (m, 1H), 7.33 (s, 1H), 7.12 (br d, J=3.1 Hz, 1H), 4.30 (dd, J=2.4, 10.1 Hz, 1H), 3.78 (s, 3H), 3.75 (br d, J=11.4 Hz, 1H), 3.57-3.48 (m, 1H), 3.17 (d, J=5.3 Hz, 4H), 2.86 (td, J=2.6, 12.3 Hz, 1H), 2.71-2.66 (m, 2H), 2.64-2.54 (m, 2H).

Step 5: 4-[4-(4-chloro-2-fluoro-phenyl)-6,7-dimethyl-pteridin-2-yl]-2-(1-methylpyrazol-4-yl)morpholine. A mixture of 4-methylbenzenesulfonic acid;2-(1-methylpyrazol-4-yl)morpholine (1.50 eq, 473 mg, 1.39 mmol) and 2-chloro-4-(4-chloro-2-fluoro-phenyl)-6,7-dimethyl-pteridine (1.00 eq, 300 mg, 0.928 mmol) in DMSO (15 mL) was added DIPEA (5.00 eq, 0.81 mL, 4.64 mmol) then stirred at 100° C. for 0.5 h. LCMS showed starting material consumed and desired product (454.2, [M+H]⁺, ESI⁺) is formed. LCMS showed starting material consumed and desired product (39%, Rt: 0.859 min; [M+H]⁺=454.2 at 220 nm) was formed. The reaction mixture was diluted with water (30 mL) and EtOAc (30 mL), then the mixture was filtered and the filtrate was extracted with EtOAc (30 mL five times). The combined organic layers were washed with brine (20 mL), dried over [Drying Na2SO4] and purified by prep-HPLC (Column: Phenomenex luna C18 150*40 mm*15 um; Condition: water(FA)-ACN) and lyophilized to give 4-[4-(4-chloro-2-fluoro-phenyl)-6,7-dimethyl-pteridin-2-yl]-2-(1-methylpyrazol-4-yl)morpholine (130 mg, 0.284 mmol, 30.60% yield) as yellow solid. [M+H]⁺=454.2. Retention time=0.859 min

Step 6: 4-[4-(4-chloro-2-fluoro-phenyl)-6,7-dimethyl-pteridin-2-yl]-2-(1-methylpyrazol-4-yl)morpholine (1.00 eq, 130 mg, 0.28 mmol) was separated by SFC (Column: DAICEL CHIRALCEL OJ (250 mm*30 mm, 10 um); Condition: 0.1% NH₃H₂O MEOH) and lyophilized to give (2S)-4-[4-(4-chloro-2-fluoro-phenyl)-6,7-dimethyl-pteridin-2-yl]-2-(1-methylpyrazol-4-yl)morpholine (16 mg, 0.0344 mmol, 12.00% yield) (Peak 2 in SFC) as yellow solid and (2R)-4-[4-(4-chloro-2-fluoro-phenyl)-6,7-dimethyl-pteridin-2-yl]-2-(1-methylpyrazol-4-yl)morpholine (14 mg, 0.0306 mmol, 10.69% yield) (Peak 1 in SFC as yellow solid. 1-1202: Rt: 0.679 min, m/z=454.0 [M+H]⁺, 100% purity at 220 nm. ¹H NMR (400 MHz, CDCl3) δ=7.58 (t, J=7.8 Hz, 1H), 7.48 (s, 1H), 7.36 (s, 1H), 7.23 (dd, J=1.9, 8.1 Hz, 1H), 7.17 (br d, J=2.0 Hz, 1H), 5.04-4.86 (m, 1H), 4.76 (br d, J=13.5 Hz, 1H), 4.53 (dd, J=2.6, 10.1 Hz, 1H), 4.02 (br dd, J=1.5, 11.5 Hz, 1H), 3.83 (s, 3H), 3.73 (dt, J=2.8, 11.5 Hz, 1H), 3.34-3.25 (m, 1H), 3.21 (dd, J=10.4, 13.4 Hz, 1H), 2.64 (s, 3H), 2.52 (s, 3H). ¹⁹F NMR (377 MHz, CDCl3) δ=−108.43 (s, 1F).

### Synthesis of 1-1210

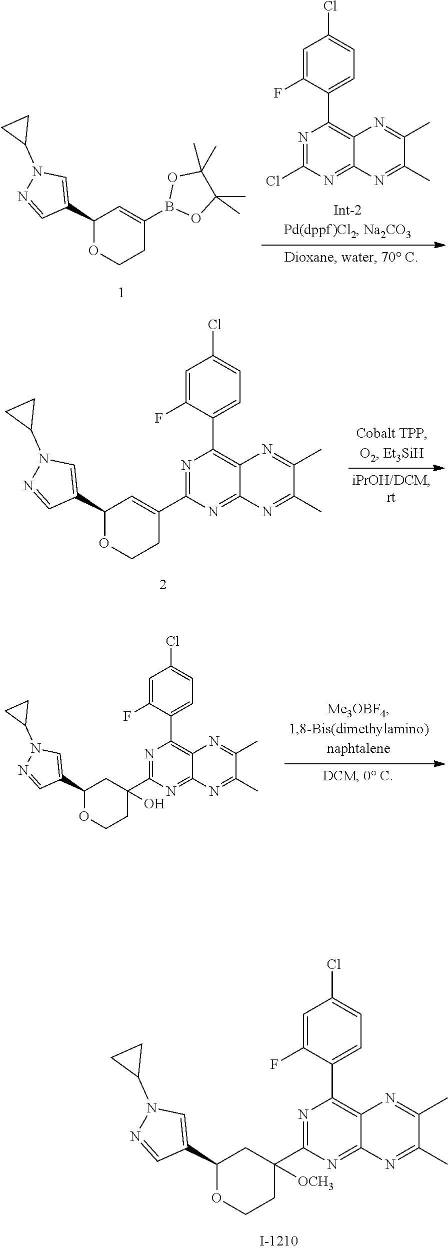

Step 1: To a solution of compound 1 (900 mg, 2.85 mmol, 1.00 eq) and INT-2 (920 mg, 2.85 mmol, 1.0 eq) in 1,4-dioxane (10 mL) and H₂O (1 mL) were added Pd(dppf)Cl₂ (104 mg, 0.14 mmol, 0.05 eq) and Na₂CO₃ (603 mg, 5.69 mmol, 2 eq). The reaction mixture was stirred at 70° C. under N₂ for 4 hours. LCMS: RT=1.50 min showed (R)-4-(4-chloro-2-fluorophenyl)-2-(6-(1-cyclopropyl-1H-pyrazol-4-yl)-3,6-dihydro-2H-pyran-4-yl)-6,7-dimethylpteridine was consumed and one main peak with desired m/z was detected. The mixture was filtered and the filtrate was evaporated. The crude product was purified by flash chromatography (Petroleum ether:EtOAc=1:1) to afford the desired product as a brown solid (900 mg, 66.2%).

Step 2: To a solution of compound 2 (900 mg, 1.90 mmol, 1.0 eq) in CH₂Cl₂ (10 mL) and i-PrOH (10 mL) were added Cobalt TPP (128 mg, 0.19 mmol. 0.1 eq) and Et₃SiH (440 mg, 3.80 mmol, 2.0 eq) at 0° C. Then the reaction mixture was stirred at 0° C. for 1 hour under O₂. LC-MS: RT=1.50 min showed (2R)-4-(4-(4-chloro-2-fluorophenyl)-6,7-dimethylpteridin-2-yl)-2-(1-cyclopropyl-1H-pyrazol-4-yl)tetrahydro-2H-pyran-4-ol was consumed and one main peak with desired m/z was detected. The solvent was evaporated and the residue was purified by flash column chromatography (eluting with DCM: MeOH=95:5) to afford (2R)-4-(4-(4-chloro-2-fluorophenyl)-6,7-dimethylpteridin-2-yl)-2-(1-cyclopropyl-1H-pyrazol-4-yl)tetrahydro-2H-pyran-4-ol as a brown solid (500 mg, 53%).

Step 3: To a solution of (2R)-4-(4-(4-chloro-2-fluorophenyl)-6,7-dimethylpteridin-2-yl)-2-(1-cyclopropyl-1H-pyrazol-4-yl)tetrahydro-2H-pyran-4-ol (300 mg, 0.61 mmol, 1.0 eq) in CH₂Cl₂ (10 mL) were added Me₃OBF₄ (108 mg, 0.73 mmol. 1.2 eq) and 1,8-bis(dimethylamino)naphthalene (257 mg, 1.20 mmol, 2.0 eq) at 0° C. Then the reaction mixture was stirred at 0° C. for 1 hour under N₂. LC-MS showed 4-(4-chloro-2-fluorophenyl)-2-((2R)-2-(1-cyclopropyl-1H-pyrazol-4-yl)-4-methoxytetrahydro-2H-pyran-4-yl)-6,7-dimethylpteridine was consumed and one main peak with desired m/z was detected. The solvent was evaporated and the residue was purified by prep-HPLC (ACN-H₂O (0.1% TFA); gradient: 5-95) and lyophilized to afford 4-(4-chloro-2-fluorophenyl)-2-((2R)-2-(1-cyclopropyl-1H-pyrazol-4-yl)-4-methoxytetrahydro-2H-pyran-4-yl)-6,7-dimethylpteridine as a white solid (31.1 mg, 10%). LCMS: (M+H)⁺=509.2. Retention time=1.018 min. HPLC: purity=95.664% (254 nm); purity=98.768% (214 nm). Retention time=2.833 min. ¹H NMR (400 MHz, DMSO) δ 8.60 (s, 1H), 8.59 (s, 1H), 7.80 (t, J=8.0 Hz, 1H), 7.72-7.55 (m, 2H), 4.92-4.89 (m, 1H), 4.13 (s, 3H), 4.06-3.99 (m, 1H), 3.84-3.81 (m, 1H), 2.79 (s, 3H), 2.68 (s, 3H), 2.27-1.95 (m, 2H), 1.36-1.19 (m, 4H).

### Synthesis of Compound I-1216

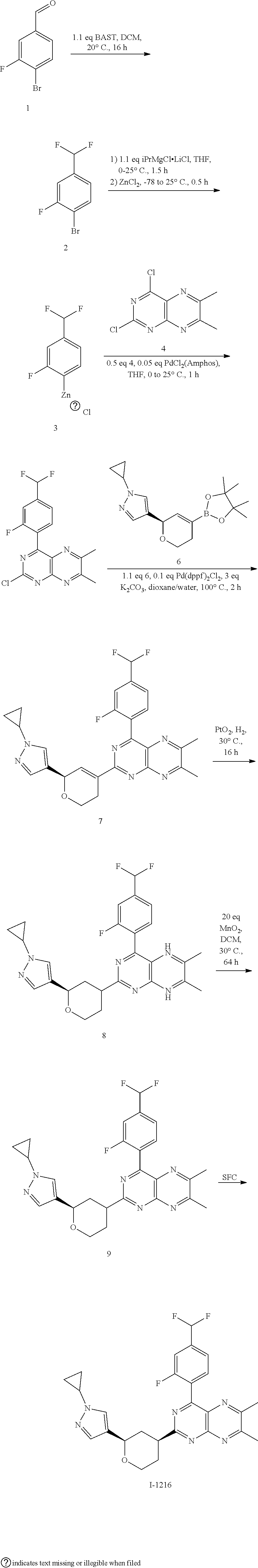

Step 1: To a solution of 4-bromo-3-fluoro-benzaldehyde (1.00 eq, 10.00 g, 49.3 mmol) in DCM (100 mL) was added BAST (1.11 eq, 10 mL, 54.8 mmol) at 0° C., the mixture was stirred at 25° C. for 12 hours. TLC (PE, UV) indicated starting material was consumed completely and one major new spot (R_{f}=0.5) formed. The reaction solution was concentrated under reduced pressure. The residue was purified with silica gel column chromatography (Petroleum ether) to give 1-bromo-4-(difluoromethyl)-2-fluoro-benzene (7900 mg, 35.1 mmol, 71.28% yield) as colorless oil ¹H NMR (400 MHz, CDCl₃) δ ppm 6.44-6.75 (m, 1H) 7.18 (d, J=7.88 Hz, 1H) 7.24-7.31 (m, 1H) 7.65 (t, J=7.50 Hz, 1H)

Step 2: To a solution of 1-bromo-4-(difluoromethyl)-2-fluoro-benzene (1.00 eq, 500 mg, 2.22 mmol) in THF (5 mL) was added iPrMgCl—LiCl (1.10 eq, 1.9 mL, 2.44 mmol) at 0° C. under N₂, the mixture was stirred at 20° C. for 1.5 h. ZnCl₂ (1.20 eq, 3.8 mL, 2.67 mmol) was added at −78° C. under N₂ and the mixture was stirred at 20° C. for 1 h. The reaction mixture was used directly for the next step.

Step 3: A sealed bottle under a N₂ atmosphere was charged with 2,4-dichloro-6,7-dimethyl-pteridine (1.00 eq, 150 mg, 0.655 mmol) and PdCl₂ (Amphos) (0.0500 eq, 23 mg, 0.0327 mmol) and THF (2 mL) and purged with N₂ for three times, then cooled to 0° C., chloro-[4-(difluoromethyl)-2-fluoro-phenyl] zinc (2.00 eq, 6.5 mL, 1.31 mmol) was added dropwise to the reaction solution at 0° C., then warmed to 25° C. and stirred for 1 hour. The reaction solution was changed from yellow to dark brown. LCMS (5-95AB/1.5 min): RT=0.890 min, 399.2=[M+H]⁺, ESI⁺ showed starting material was consumed completely and desired product was detected. The reaction solution was quenched with saturated NH₄Cl solution (10 mL), then extracted with EtOAc (20 mL*3). The combined organic layers was evaporated under reduced pressure to give the residue, which was then purified with flash column (TLC, PE:EA=3:1, R_{f}=0.5) and dried in vacuo to give 2-chloro-4-[4-(difluoromethyl)-2-fluoro-phenyl]-6,7-dimethyl-pteridine (220 mg, 0.650 mmol, 99.19% yield) as yellow solid ¹H NMR (400 MHz, DMSO-d6) δ ppm 2.68 (s, 3H) 2.81 (s, 3H) 7.04-7.34 (m, 1H) 7.64-7.71 (m, 2H) 7.88 (t, J=7.40 Hz, 1H).

Step 4: To a solution of 2-chloro-4-[4-(difluoromethyl)-2-fluoro-phenyl]-6,7-dimethyl-pteridine (1.00 eq, 150 mg, 0.443 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 154 mg, 0.487 mmol) and K₂CO₃ (3.00 eq, 112 mg, 1.33 mmol) in 1,4-Dioxane (2 mL) and Water (0.2000 mL) was added [1,1′-Bis(diphenylphosphino)ferrocene]dichloropalladium(II) (0.0909 eq, 29 mg, 0.0403 mmol) at 20° C. The mixture was stirred at 100° C. for 2 hours. LCMS (5-95AB/1.5 min): RT=0.974 min, 493.1=[M+H]⁺, ESI⁺ showed starting material was consumed completely and desired mass was detected. The mixture was concentrated under reduced pressure to get the crude residue. The residue was purified via column chromatography on silica gel (PE:EA=1/0 to 0/1) to give 2-[(6R)-6-(1-cyclopropylpyrazol-4-yl)-3,6-dihydro-2H-pyran-4-yl]-4-[4-(difluoromethyl)-2-fluoro-phenyl]-6,7-dimethyl-pteridine (240 mg, 0.467 mmol) as brown solid. ¹H NMR (400 MHz, CDCl₃) δ ppm 0.96-1.03 (m, 2H) 1.09-1.14 (m, 2H) 2.73 (s, 3H) 2.86 (s, 3H) 2.92-3.04 (m, 2H) 3.57 (dt, J=7.24, 3.53 Hz, 1H) 3.95 (ddd, J=11.58, 6.82, 4.95 Hz, 1H) 4.10-4.18 (m, 1H) 5.46 (d, J=2.32 Hz, 1H) 6.60-6.91 (m, 1H) 7.42 (d, J=9.78 Hz, 1H) 7.49 (s, 3H) 7.67 (s, 1H) 7.85 (t, J=7.34 Hz, 1H).

Step 5: To a solution of 2-[(6R)-6-(1-cyclopropylpyrazol-4-yl)-3, 6-dihydro-2H-pyran-4-yl]-4-[4-(difluoromethyl)-2-fluoro-phenyl]-6,7-dimethyl-pteridine (1.00 eq, 240 mg, 0.487 mmol) in Ethanol (5 mL) was added PtO₂ (0.434 eq, 48 mg, 0.211 mmol) under N₂. The suspension was degassed under vacuum and purged with H₂ several times. The mixture was stirred under H₂ (15 psi) at 30° C. for 16 hours. LCMS (5-95AB/1.5 min): RT=0.474 min, 499.3=[M+H]⁺, ESI⁺ showed starting material was consumed completely and one major peak with desired mass was detected. The reaction mixture was filtered and concentrated under reduced pressure to give 2-[(2R)-2-(1-cyclopropylpyrazol-4-yl) tetrahydropyran-4-yl]-4-[4-(difluoromethyl)-2-fluoro-phenyl]-6, 7-dimethyl-5,6,7,8-tetrahydropteridine (230 mg, 0.461 mmol, 94.67% yield) as yellow oil. The crude product was used to next step directly. [M+H]⁺=499.3; purity=97.6% (220 nm). Retention time=0.474 min.

Step 6: The mixture of 2-[(2R)-2-(1-cyclopropylpyrazol-4-yl)tetrahydropyran-4-yl]-4-[4-(difluoromethyl)-2-fluoro-phenyl]-6,7-dimethyl-5,6,7,8-tetrahydropteridine (1.00 eq, 230 mg, 0.461 mmol) in DCM (10 mL) was added MnO₂ (20.0 eq, 802 mg, 9.23 mmol), then the reaction was stirred at 30° C. for 16 h. LCMS (5-95AB/1.5 min): RT=0.845 min, 495.3=[M+H]⁺, ESI⁺ showed 60% of desired compound was detected. The reaction was stirred at 30° C. for another 48 h. LCMS (5-95AB/1.5 min): RT=0.954 min, 495.1=[M+H]⁺, ESI⁺ showed 90% of desired compound was detected. The reaction mixture was filtered and concentrated under reduced pressure to give a residue. The residue was purified by prep-HPLC (Phenomenex Luna C18 150*25 mm*10 um, water (FA)-ACN) to give 2-[(2R)-2-(1-cyclopropylpyrazol-4-yl)tetrahydropyran-4-yl]-4-[4-(difluoromethyl)-2-fluoro-phenyl]-6,7-dimethyl-pteridine (140 mg, 0.283 mmol, 61.37% yield) as yellow solid, which LCMS: [M+H]⁺=495.1; purity=98.7% (220 nm). Retention time=0.955 min.

Step 7: 2-[(2R,4S)-2-(1-cyclopropylpyrazol-4-yl)tetrahydropyran-4-yl]-4-[4-(difluoromethyl)-2-fluoro-phenyl]-6,7-dimethyl-pteridine 1-1216.2-[(2R)-2-(1-cyclopropylpyrazol-4-yl)tetrahydropyran-4-yl]-4-[4-(difluoromethyl)-2-fluoro-phenyl]-6,7-dimethyl-pteridine (1.00 eq, 140 mg, 0.284 mmol) was purified by SFC (DAICEL CHIRALPAK AD (Column: Chiralpak AD-3 50×4.6 mm I.D., 3 um Mobile phase: Phase A for CO₂, and Phase B for IPA (0.05% DEA); Gradient elution: 40% IPA (0.05% DEA) in CO₂; Flow rate: 3 mL/min; Detector: PDA; Column Temp: 35C; Back Pressure: 100 Bar). 2-[(2R,4S)-2-(1-cyclopropylpyrazol-4-yl)tetrahydropyran-4-yl]-4-[4-(difluoromethyl)-2-fluoro-phenyl]-6,7-dimethyl-pteridine (36 mg, 0.0702 mmol, 24.70% yield) was obtained as yellow solid. LCMS: (M+H)⁺=495.3; purity=97.8% (220 nm). Retention time=0.848 min. ¹H NMR (400 MHz, DMSO-d6) δ ppm 0.80-0.86 (m, 2H) 0.91-1.03 (m, 6H) 1.19 (d, J=6.25 Hz, 3H) 1.62 (tt, J=8.27, 4.99 Hz, 1H) 2.63 (d, J=3.75 Hz, 6H) 2.74 (dd, J=12.01, 10.76 Hz, 1H) 2.95-3.05 (m, 1H) 3.69 (tt, J=7.38, 3.69 Hz, 1H) 3.90-4.00 (m, 1H) 4.38 (br d, J=12.38 Hz, 1H) 4.51 (br d, J=12.38 Hz, 1H) 4.73 (dd, J=10.57, 2.31 Hz, 1H) 6.99 (s, 1H) 7.45 (s, 1H) 7.82 (s, 1H).

### Synthesis of I-1221

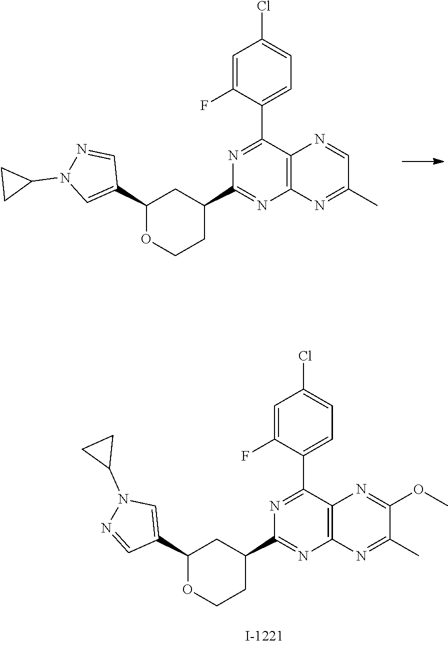 [SMILES: Cc1cnc2c(-c3ccc(Cl)cc3F)nc([C@H]3CCO[C@@H](c4cnn(C5CC5)c4)C3)nc2n1 ; COc1nc2c(-c3ccc(Cl)cc3F)nc([C@H]3CCO[C@@H](c4cnn(C5CC5)c4)C3)nc2nc1C]

To a solution of 4-(4-chloro-2-fluoro-phenyl)-7-methyl-2-[rac-(2S,4R)-2-(1-cyclopropylpyrazol-4-yl)-tetrahydropyran-4-yl]pteridine (1.00 eq, 50 mg, 0.108 mmol) in Methanol (2 mL) was added CAN (1.50 eq, 88 mg, 0.161 mmol) in N2, the mixture was stirred at 60° C. for 4 h. The mixture was concentrated to give a crude product in vacuum. The crude product was purified by prep-HPLC (FA, column: Phenomenex Luna C18 150*25 mm*10 um; mobile phase: [water(FA)-ACN];B %: 54%-84%,10 min). The purified solution was lyophilized. 4-(4-chloro-2-fluoro-phenyl) −6-methoxy-7-methyl-2-[rac-(2S,4R)-2-(1-cyclopropylpyrazol-4-yl)tetrahydropyran-4-yl]pteridine (2.1 mg, 0.00400 mmol, 3.72% yield) was obtained as white solid. LC-MS: Rt: 0.994 min; 495.1=[M+H]⁺, ESI⁺; 94.3% purity at 220 nm. ¹H NMR (400 MHz, CDCl3) δ=7.69-7.57 (m, 1H), 7.43 (s, 2H), 7.27 (dd, J=1.8, 8.1 Hz, 1H), 7.21 (d, J=1.9 Hz, 1H), 4.48 (dd, J=2.0, 11.4 Hz, 1H), 4.26-4.14 (m, 1H), 3.99-3.88 (m, 3H), 3.78-3.64 (m, 1H), 3.54-3.39 (m, 2H), 2.69 (s, 3H), 2.44-2.29 (m, 1H), 2.17-2.05 (m, 3H), 1.09-0.98 (m, 2H), 0.94-0.85 (m, 2H).

### Synthesis of Compounds I-1226

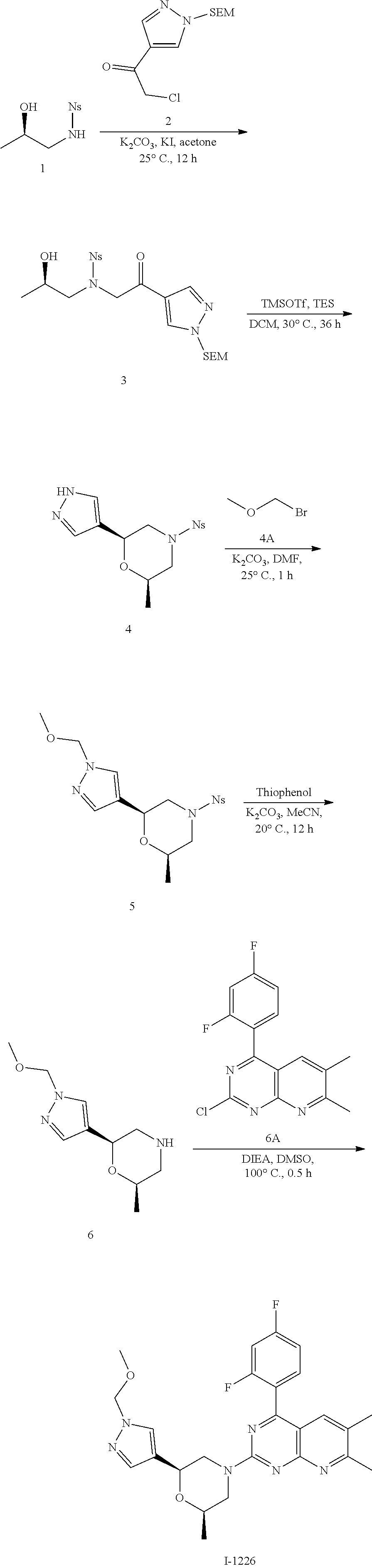

Step 1: To a solution of N-[(2R)-2-hydroxypropyl]-4-nitro-benzenesulfonamide (1.00 eq, 0.95 g, 3.64 mmol) and K₂CO₃ (1.50 eq, 0.75 g, 5.46 mmol) in Acetone (40 mL) was added 2-chloro-1-[1-(2-trimethylsilylethoxymethyl)pyrazol-4-yl]ethanone (1.00 eq, 1.00 g, 3.64 mmol) at 0° C., the mixture was stirred at 25° C. for 12 h. LCMS showed the starting material was consumed completely and a major peak with desired MS (57%, MS: 499.2 [M+H]⁺, ESI pos). The reaction mixture was partitioned between EtOAc (200 mL) and water (200 mL). The combined organic layers were washed with brine (50 mL), dried over Na₂SO₄, filtered and concentrated under reduced pressure to give a residue. The residue was purified by prep-TLC (SiO₂, DCM:MeOH=20:1; R_{f}=0.4) to afford N-[(2R)-2-hydroxypropyl]-4-nitro-N-[2-oxo-2-[1-(2-trimethylsilylethoxymethyl) pyrazol-4-yl] ethyl] benzenesulfonamide 3 (400 mg, 0.802 mmol, 22.05% yield) as white solid. LCMS (M+H)⁺=499.2; purity=98% (220 nm). Retention time=0.943 min. ¹H NMR (400 MHz, DMSO-d₆) δ ppm −0.04 (s, 9H) 0.82-0.87 (m, 3H) 1.00 (d, J=6.25 Hz, 4H) 3.08 (dd, J=14.32, 7.69 Hz, 2H) 3.24 (d, J=3.88 Hz, 1H) 3.51-3.60 (m, 3H) 4.72 (d, J=4.75 Hz, 1H) 4.80 (d, J=2.50 Hz, 2H) 5.45 (s, 2H) 8.05 (s, 1H) 8.09 (d, J=8.76 Hz, 2H) 8.38 (d, J=8.88 Hz, 2H) 8.67 (s, 1H).

Step 2: To a solution of N-[(2R)-2-hydroxypropyl]-4-nitro-N-[2-oxo-2-[1-(2-trimethylsilylethoxymethyl)pyrazol-4-yl]ethyl]benzenesulfonamide (1.00 eq, 390 mg, 0.782 mmol) in DCM (4 mL) was added TES (15.0 eq, 2690 mg, 11.7 mmol) and TMSOTf (5.00 eq, 0.71 mL, 3.91 mmol) at 0° C., the mixture was stirred at 30° C. for 12 h. LCMS showed the starting material was consumed completely and a major peak with desired MS (MS: 353.1 [M+H]⁺, ESI pos). The reaction mixture was filtered and concentrated under reduced pressure to give a residue. The residue was purified by prep-TLC (SiO₂, DCM:MeOH=20:1; R_{f}=0.4) to afford (2R,6S)-2-methyl-4-(4-nitrophenyl) sulfonyl-6-(1H-pyrazol-4-yl) morpholine 4 (205 mg, 0.582 mmol, 74.38% yield) as white solid. LCMS (M+H)⁺=353.1; purity=99% (220 nm). Retention time=0.810 min. ¹H NMR (400 MHz, DMSO-d6) δ ppm 1.10 (d, J=6.13 Hz, 3H) 2.06 (t, J=10.94 Hz, 1H) 3.65 (br t, J=9.69 Hz, 2H) 3.71-3.80 (m, 1H) 4.03 (q, J=7.13 Hz, 1H) 4.11 (q, J=7.13 Hz, 1H) 4.61 (dd, J=10.44, 2.31 Hz, 1H) 5.75 (s, 1H) 7.49-7.66 (m, 1H) 8.05 (d, J=8.76 Hz, 2H) 8.44 (d, J=8.88 Hz, 2H).

Step 3: To a solution of bromo (methoxy) methane (1.70 eq, 60 mg, 0.482 mmol) and K₂CO₃ (2.00 eq, 78 mg, 0.568 mmol) in DMF (3 mL) was added (2R,6S)-2-methyl-4-(4-nitrophenyl) sulfonyl-6-(1H-pyrazol-4-yl) morpholine (1.00 eq, 100 mg, 0.284 mmol), the mixture was stirred at 25° C. for 1 h. LCMS showed the starting material was consumed completely and a major peak with desired product mass (96%, MS: 397.1 [M+H]⁺, ESI pos). The reaction mixture was partitioned between EtOAc (40 mL) and water (40 mL). The separated organic layer was washed with water, dried over Na₂SO₄ and evaporated to dryness. The reaction mixture was filtered and concentrated under reduced pressure to give a residue. The residue was purified by prep-TLC (SiO₂, DCM:MeOH=20:1; R_{f}=0.4) to afford (2S, 6R)-2-[1-(methoxymethyl)pyrazol-4-yl]-6-methyl-4-(4-nitrophenyl) sulfonyl-morpholine 5 (130 mg, 0.328 mmol, 115.55% yield) as white solid. Yield >100% because there are some DMF shown in H NMR. LCMS (M+H)⁺=397.1; purity=97% (220 nm). Retention time=0.841 min. ¹H NMR (400 MHz, CDCl3) δ ppm 1.23 (d, J=6.25 Hz, 3H) 2.13 (t, J=10.82 Hz, 1H) 2.30 (t, J=11.01 Hz, 1H) 3.33 (s, 3H) 3.68-3.73 (m, 1H) 3.81 (dd, J=11.44, 2.31 Hz, 1H) 3.85-3.91 (m, 1H) 4.70 (dd, J=10.44, 2.56 Hz, 1H) 5.34 (s, 2H) 7.52 (d, J=11.88 Hz, 2H) 7.96 (d, J=8.88 Hz, 2H) 8.42 (d, J=8.88 Hz, 2H).

Step 4: To a solution of (2S,6R)-2-[1-(methoxymethyl) pyrazol-4-yl]-6-methyl-4-(4-nitrophenyl) sulfonyl-morpholine (1.00 eq, 100 mg, 0.252 mmol), K₂CO₃ (5.00 eq, 174 mg, 1.26 mmol) in MeCN (5 mL) was added thiophenol (5.00 eq, 139 mg, 1.26 mmol), then the mixture was stirred at 25° C. for 12 h. LCMS showed starting material consumed and desired product (212.1, [M+H]⁺, ESI⁺) is formed. The combined reaction mixture was poured into water (20 mL), and then extracted with EtOAc (20 mL, three times). LCMS showed desired compound mass in aqueous phase which lyophilized to give crude (2S,6R)-2-[1-(methoxymethyl) pyrazol-4-yl]-6-methyl-morpholine 6 (36 mg, 0.170 mmol, 67.55% yield) as yellow foam. LCMS: (M+H)⁺=212.2; purity=83% (220 nm). Retention time=0.148 min.

Step 5: To a solution of (2S,6R)-2-[1-(methoxymethyl)pyrazol-4-yl]-6-methyl-morpholine (1.00 eq, 36 mg, 0.170 mmol) and 2-chloro-4-(2,4-difluorophenyl)-6,7-dimethyl-pyrido[2,3-d]pyrimidine (1.00 eq, 52 mg, 0.170 mmol) in DMSO (0.5000 mL) was added DIEA (4.00 eq, 88 mg, 0.682 mmol). The mixture was stirred at 100° C. for 0.5 hour. LCMS showed the starting material was consumed completely and a major peak with desired product mass (Rt: 0.813 min, m/z: 481.2 [M+H]⁺, 62% purity at 254 nm). The reaction was filtered and the filtrate was purified by prep-HPLC (flow: 25 mL/min; gradient: from 22-52% water (0.1% FA)-ACN over 7 min; column: Unisil 3-100 C18 Ultra 150 * 50 mm*3 um) and lyophilized to afford (2S,6R)-4-[4-(2,4-difluorophenyl)-6,7-dimethyl-pyrido[2,3-d]pyrimidin-2-yl]-2-[1-(methoxymethyl)pyrazol-4-yl]-6-methyl-morpholine (25 mg, 0.0524 mmol, 30.78% yield) as yellow solid. LCMS (M+H)⁺=481.2; purity=100% (220 nm). Retention time=0.812 min. HPLC: Retention time: 1.751 min, 92% purity at 220 nm. 1H NMR (400 MHz, DMSO-d6) δ ppm 1.23 (br d, J=5.50 Hz, 3H) 2.29 (s, 3H) 2.57 (s, 3H) 2.77 (dd, J=13.01, 10.63 Hz, 1H) 3.04 (br t, J=11.88 Hz, 1H) 3.23 (s, 3H) 3.73-3.82 (m, 1H) 4.60 (dd, J=10.63, 1.75 Hz, 1H) 4.70-4.87 (m, 2H) 5.36 (s, 2H) 7.28-7.36 (m, 1H) 7.46-7.54 (m, 1H) 7.56-7.63 (m, 2H) 7.66-7.76 (m, 1H) 7.98 (s, 1H).

### Synthesis of Compound I-1236

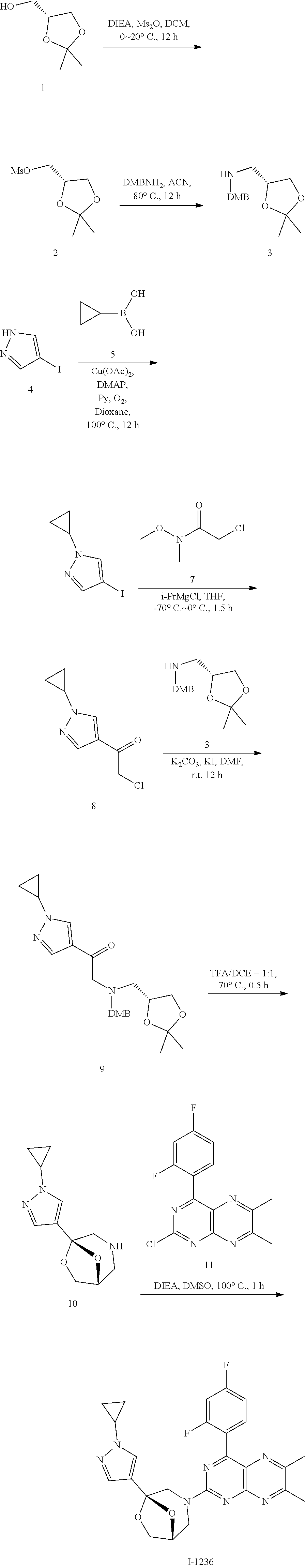

Step 1: To a mixture of [(4R)-2,2-dimethyl-1,3-dioxolan-4-yl]methanol (1.00 eq, 1.00 g, 7.57 mmol), DIPEA (3.00 eq, 4.0 mL, 22.7 mmol) in DCM (10 mL) was added methylsulfonyl methanesulfonate (1.50 eq, 1.97 g, 11.3 mmol) at 0° C., then the mixture was stirred at 25° C. for 12 hr. The mixture was poured into water (50 mL) and extracted with DCM (3*50 mL), the organic phase was concentrated to give a residue. The solution was purified by column on silica (3 g SiO₂ cartridge, PE:EA=1:1, detection at phosphomolybdic acid) and concentrated under reduced pressure to give a yellow oil. [(4S)-2,2-dimethyl-1,3-dioxolan-4-yl]methyl methanesulfonate (1150 mg, 5.20 mmol, 68.67% yield) was obtained as a yellow oil and used to the next step. ¹H NMR (400 MHz, CDCl3) δ=4.44-4.34 (m, 1H), 4.23 (d, J=5.3 Hz, 2H), 4.11 (dd, J=6.5, 8.8 Hz, 1H), 3.84 (dd, J=5.4, 8.7 Hz, 1H), 3.08 (s, 3H), 1.45 (s, 3H), 1.38 (s, 3H).

Step 2: To a yellow solution of [(4S)-2,2-dimethyl-1,3-dioxolan-4-yl]methyl methanesulfonate (1.00 eq, 500 mg, 2.38 mmol) in MeCN (6.8 mL) was added (2,4-dimethoxyphenyl)methanamine (4.00 eq, 1591 mg, 9.51 mmol) to give a dark yellow solution, then the mixture was stirred at 80° C. for 12 hr. LCMS showed the starting material was consumed completely and 41% desired MS (282.2 [M+1]⁺, ESI pos) was found. The mixture was cooled to the room temperature. The combined mixture was poured into water (20 mL) and extracted by ethyl acetate (3*50 mL), the organic phase was concentrated to give a residue. The solution was purified by column on silica (2 g SiO₂ cartridge, EA/MeOH=1.2%, 1% NH₃·H₂O, detection at 254 nm) and concentrated under reduced pressure to give a brown oil. 1-(2,4-dimethoxyphenyl)-N-[[(4R)-2,2-dimethyl-1,3-dioxolan-4-yl]methyl]methanamine (400 mg, 1.39 mmol, 58.59% yield) was obtained as a brown oil. ¹H NMR (400 MHz, CDCl3) δ=7.13 (d, J=8.0 Hz, 1H), 6.49-6.40 (m, 2H), 4.31-4.20 (m, 1H), 4.04 (dd, J=6.5, 7.9 Hz, 1H), 3.81 (d, J=3.8 Hz, 6H), 3.76 (s, 2H), 3.71-3.63 (m, 1H), 2.77-2.64 (m, 2H), 1.40 (s, 3H), 1.35 (s, 3H).

Step 3: To a solution of 4-iodo-1H-pyrazole (1.00 eq, 10.00 g, 51.6 mmol) in 1,4-Dioxane (200 mL) was added cyclopropylboronic acid (2.00 eq, 8.86 g, 103 mmol), Cu(OAc)₂ (1.00 eq, 9.36 g, 51.6 mmol), DMAP (4.00 eq, 25.16 g, 206 mmol) and pyridine (2.50 eq, 10 mL, 129 mmol). The resulting mixture was stirred at 100° C. for 16 h under oxygen atmosphere. Color of the solution was changed from blue to black. TLC (PE:EA=1: 1) showed starting material was consumed completely and two new spots was formed. LCMS showed starting material was consumed completely and 96% desired product was formed. The mixture was poured into water (1000 mL) and was extracted with EA (1000 mL×3). The combined organic layers were washed with brine (1000 mL×1), dried over Na₂SO₄ and concentrated to give the crude. The crude was purified by column chromatography on silica gel eluted with (PE:EA=2: 1-1: 1) to give 1-cyclopropyl-4-iodo-pyrazole (11.40 g, 48.7 mmol, 94.4% yield) as a light-yellow oil. ¹H NMR (400 MHz, CDCl3) δ=7.52 (s, 1H), 7.49 (s, 1H), 3.62 (tt, J=3.7, 7.3 Hz, 1H), 1.15-1.10 (m, 2H), 1.08-1.01 (m, 2H).

Step 4: To a solution of 1-cyclopropyl-4-iodo-pyrazole (1.00 eq, 10.40 g, 44.4 mmol) in THF (200 mL) was added dropwise i-PrMgCl (1.50 eq, 33 mL, 66.7 mmol) at −70° C. under N₂ and then the reaction mixture was stirred at −70° C. for 30 mins, then 2-CHLORO-N-METHOXY-N-METHYLACETAMIDE (1.20 eq, 7.34 g, 53.3 mmol) in THF (20 mL) was added dropwise and stirred at 0° C. for 1 hr. LCMS showed the reactant was consumed and the desired mass was detected, the reaction solution was quenched with saturated NH₄C1, poured into H₂O, extracted with EtOAc and evaporated under reduced pressure to give the residue, which was then purified with Flash column (PE:EA=0-60%) and evaporated under reduced pressure to give 2-chloro-1-(1-cyclopropylpyrazol-4-yl)ethanone (8.10 g, 43.9 mmol, 98.73% yield) as colorless oil. After cooled to room temperature, the product turned into off-white solid. ¹H NMR (400 MHz, CDCl3) δ=7.99 (s, 1H), 7.87 (s, 1H), 4.33 (s, 2H), 3.59 (tt, J=3.8, 7.3 Hz, 1H), 1.13-1.07 (m, 2H), 1.07-1.00 (m, 2H).

Step 5: To a purple mixture of 2-chloro-1-(1-cyclopropylpyrazol-4-yl)ethanone (1.00 eq, 170 mg, 0.921 mmol) in DMF (10 mL) was added 1-(2,4-dimethoxyphenyl)-N-[[(4R)-2,2-dimethyl-1,3-dioxolan-4-yl]methyl]methanamine (1.50 eq, 389 mg, 1.38 mmol), K₂CO₃ (2.00 eq, 255 mg, 1.84 mmol) and KI (1.50 eq, 229 mg, 1.38 mmol), then the yellow mixture was stirred at 15° C. for 12 hr. LCMS showed the starting material was consumed completely and 60% desired MS (430.3 [M+1]⁺, ESI pos) was found. The mixture was poured into water (60 mL) and extracted by ethyl acetate (3*50 mL), the organic phase was concentrated to give a residue. The solution was purified by column on silica (1 g SiO₂ cartridge, EA, detection at 254 nm) and concentrated under reduced pressure to give a yellow oil. 1-(1-cyclopropylpyrazol-4-yl)-2-[(2,4-dimethoxyphenyl)methyl-[[(4R)-2,2-dimethyl-1,3-dioxolan-4-yl]methyl]amino] ethanone (320 mg, 0.633 mmol, 68.77% yield) was obtained as a yellow oil. ¹H NMR (400 MHz, CDCl3) δ=8.08 (s, 1H), 7.92 (s, 1H), 7.19-7.09 (m, 1H), 6.45-6.42 (m, 2H), 4.34 (quin, J=6.2 Hz, 1H), 4.04-3.97 (m, 1H), 3.78 (d, J=15.5 Hz, 8H), 3.63-3.52 (m, 4H), 2.79-2.63 (m, 2H), 1.35 (d, J=4.3 Hz, 6H), 1.15-1.02 (m, 4H).

Step 6: To a mixture of 1-(1-cyclopropylpyrazol-4-yl)-2-[(2,4-dimethoxyphenyl)methyl-[[(4R)-2,2-dimethyl-1,3-dioxolan-4-yl]methyl]amino]ethanone (1.00 eq, 200 mg, 0.466 mmol) in DCE (1 mL) was added TFA (28.0 eq, 1.0 mL, 13.1 mmol), then the yellow mixture was stirred at 70° C. for 1 hr to give a red solution. LCMS showed the starting material was consumed completely and 11% desired MS (222.1[M+1]⁺, ESI pos) and 13% by product (372.1[M+1]⁺, ESI pos) was found. The reaction mixture was cooled to room temperature and concentrated to give a yellow oil. (1R,5S)-5-(1-cyclopropylpyrazol-4-yl)-6,8-dioxa-3-azabicyclo[3.2.1]octane (300 mg, 1.36 mmol, 291.18% yield) was obtained as a yellow oil.

Step 7: To a yellow solution of 2-chloro-4-(2,4-difluorophenyl)-6,7-dimethyl-pteridine (1.00 eq, 40 mg, 0.130 mmol), (1R,5S)-5-(1-cyclopropylpyrazol-4-yl)-6,8-dioxa-3-azabicyclo[3.2.1]octane (5.00 eq, 144 mg, 0.652 mmol) in DMSO (2 mL) was added DIPEA (4.00 eq, 0.091 mL, 0.522 mmol), then the mixture was stirred at 100° C. for 1 hr to give a brown solution. LCMS showed the starting material was consumed completely and 23.7% desired MS (492.0 [M+1]⁺, ESI pos) was found. The mixture was cooled to room temperature. Then the mixture was poured into water (20 mL) and extracted by ethyl acetate (3×30 mL), the combined organic layers were washed with brine, dried over anhydrous sodium sulfate, filtered and concentrated in vacuo. The solution was purified by prep-TLC (EA, Rf=0.5), the purified solution was concentrated to give a brown oil. Then the solution was lyophilized to give a brown solid. SFC showed 55% desired MS (492.2 [M+1]⁺, ESI pos) (peak 2) was found and 19% MS (532.1[M+18+Na]⁺, ESI pos, possible ring-open BP) was found and 25% desired mass (492.1[M+1]⁺, ESI pos) (peak 1) was found. The solution was re-purified by SFC (column: DAICEL CHIRALPAK AD (250 mm*30 mm, 10 um); mobile phase: 0.1% NH3·H2O ETOH; B %: 60%-60%, 9.8 min), the purified solution was concentrated to give a yellow oil. Then the solution was lyophilized to give a yellow solid. (1R,5S)-5-(1-cyclopropylpyrazol-4-yl)-3-[4-(2,4-difluorophenyl)-6,7-dimethyl-pteridin-2-yl]-6,8-dioxa-3-azabicyclo[3.2.1]octane (3.7 mg, 0.00719 mmol, 5.51% yield) was obtained as a yellow solid. The product was the peak 2 in SFC. Peak 1 mixed with impurity and was isolated unsuccessfully. 1H NMR for DP (peak 2 in SFC): LCMS. Rt: 0.899 min; m/z: 492.1 [M+H]⁺. 100% purity at 214 nm. ¹H NMR (400 MHz, CDCl3) δ=7.76-7.70 (m, 1H), 7.69-7.60 (m, 2H), 7.04 (br t, J=7.8 Hz, 1H), 7.00-6.91 (m, 1H), 4.99-4.72 (m, 3H), 4.18-3.99 (m, 2H), 3.65-3.50 (m, 2H), 3.49-3.39 (m, 1H), 2.72 (s, 3H), 2.59 (s, 3H), 1.13 (br s, 2H), 1.02 (br d, J=5.9 Hz, 2H). HPLC: Rt: 2.720 min; 94.564% purity at 214 nm.

### Synthesis of Compounds I-1241 and I-1270

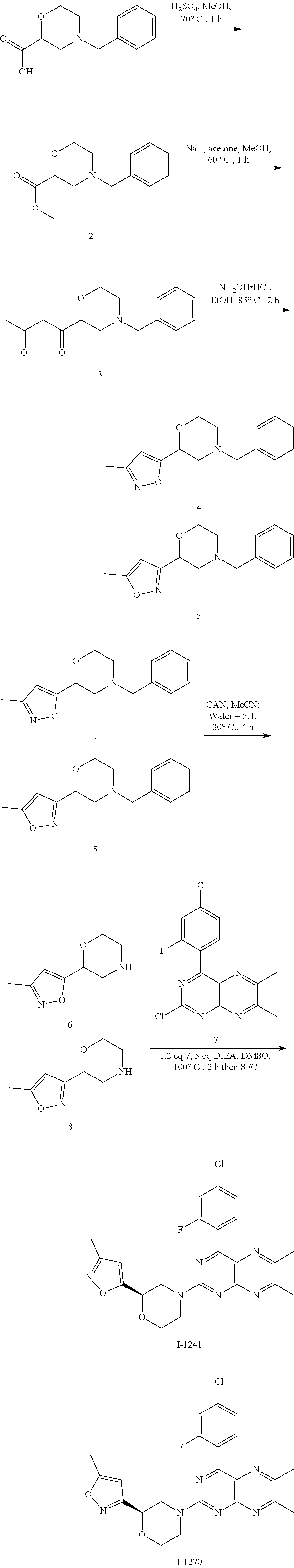

Step 1: A solution of 4-benzylmorpholine-2-carboxylic acid (1.00 eq, 5.00 g, 22.6 mmol) and H₂SO₄ (221 eq, 5.0 mL, 5000 mmol) in Methanol (100 mL) was stirred at 70° C. for 1 hour. LCMS, product: RT=0.846 min) showed the starting material was consumed completely, and 100% desired mass was detected. The reaction mixture was poured into saturated aqueous NaHCO₃ (100 mL) slowly and stirred at 0° C. for 10 min, then the mixture was extracted with EtOAc (100 mL*3) and water (100 mL*3). The organic layer was washed with brine, dried by Na₂SO₄. The solution was concentrated to give the residue. The crude product was purified by column chromatography on silica gel eluted with petroleum ether/ethyl acetate=100/1 to 0/1, (DCM:MeOH=10:1, the desired product R_{f}=0.3 (I₂)) to give 4-benzylmorpholine-2-carboxylate (4300 mg, 17.9 mmol, 79.25% yield) was obtained as colorless oil. (M+H)⁺=236.1; purity=98% (220 nm). Retention time=0.846 min. 1H NMR (400 MHz, CDCl₃) δ=7.36-7.31 (m, 4H), 7.36-7.28 (m, 1H), 4.25 (dd, J=2.9, 9.1 Hz, 1H), 4.02 (td, J=3.2, 11.4 Hz, 1H), 3.78-3.75 (m, 3H), 3.75-3.68 (m, 1H), 3.58-3.45 (m, 2H), 3.03-2.94 (m, 1H), 2.73-2.57 (m, 1H), 2.40-2.22 (m, 2H).

Step 2: To a solution of methyl 4-benzylmorpholine-2-carboxylate (1.00 eq, 2.00 g, 8.50 mmol) and acetone (2.20 eq, 1.4 mL, 18.7 mmol) in THF (40 mL) was bubbled with N₂ for 30 seconds, then NaH (2.20 eq, 748 mg, 18.7 mmol) was added slowly and stirred at 60° C. for 1 h. LCMS, rt=0.614 min) showed 70% purity desired mass ((M+H)⁺=262.1) was detected. The residue was partitioned between ethyl acetate (50*3 mL) and water (50*3 mL). The separated organic layer was washed with water, dried over Na₂SO₄ and evaporated to dryness. The crude product was purified by column chromatography on silica gel eluted with petroleum ether/ethyl acetate=100/1 to 0/1 (petroleum ether/ethyl acetate=0/1, the desired product R_{f}=0.4) to give 1-(4-benzylmorpholin-2-yl)butane-1,3-dione (800 mg, 2.91 mmol, 34.21% yield) was obtained as yellow oil: (M+H)⁺=262.1; purity=78% (220 nm). Retention time=0.614 min. 1H NMR (400 MHz, CDCl₃) δ=7.39-7.25 (m, 5H), 4.18-4.09 (m, 1H), 3.94 (br d, J=11.2 Hz, 1H), 3.78-3.61 (m, 1H), 3.60-3.49 (m, 2H), 3.07 (br d, J=11.0 Hz, 1H), 2.68 (br d, J=11.1 Hz, 1H), 2.26-2.17 (m, 1H), 2.12-2.07 (m, 3H), 2.05 (s, 1H), 1.27 (t, J=7.2 Hz, 1H).

Step 3: To a solution of 1-(4-benzylmorpholin-2-yl) butane-1,3-dione (1.00 eq, 800 mg, 3.06 mmol) in Ethanol (13 mL) was added NH₂OH HCl (1.30 eq, 277 mg, 3.98 mmol) stirred at 85° C. for 2 hours. LCMS (5-95AB/1.5 min): RT=0.320 min, 259.3=[M+H]⁺, ESI⁺ showed 97% of desired mass. The residue was evaporated to dryness for further purification. The residue was purified by flash silica gel chromatography petroleum ether/ethyl acetate=100/1 to 0/1 (DCM:MeOH=10:1, the desired product R_{f}=0.3). The purified solution was concentrated to give 4-benzyl-2-(3-methylisoxazol-5-yl) morpholine and 4-benzyl-2-(5-methylisoxazol-3-yl) morpholine (500 mg, 1.84 mmol, 60.06% yield) was obtained as white gum, LCMS (M+H)⁺=259.1; purity=95% (220 nm). Retention time=0.905 min. 1H NMR (400 MHz, DMSO-d6) δ=7.63-7.42 (m, 5H), 6.43-6.23 (m, 1H), 5.20-4.88 (m, 1H), 4.53-4.29 (m, 1H), 2.71-2.63 (m, 2H), 2.42-2.39 (m, 2H), 2.34-2.31 (m, 2H), 2.25-2.20 (m, 2H), 1.99 (s, 1H), 1.94-1.89 (m, 1H), 1.26-1.22 (m, 1H).

Step 4: To a solution of 4-benzyl-2-(3-methylisoxazol-5-yl)morpholine (1.00 eq, 500 mg, 1.94 mmol) in MeCN (2.5 mL) and Water (0.5 mL) was added Ammonium cerium (IV) nitrate (2.00 eq, 2122 mg, 3.87 mmol). The mixture was stirred at 25° C. for 2 hours. LCMS (0-60AB/1.5 min): RT=0.115 min, 169.2=[M+H]⁺, ESI⁺ showed 80% of desired mass. The residue was evaporated to dryness for further purification. The residue was purified by flash silica gel chromatography petroleum ether/ethyl acetate=100/1 to 0/1 (DCM:MeOH=10:1, the desired product R_{f}=0.3). The purified solution was concentrated to give 2-(3-methylisoxazol-5-yl)morpholine and 2-(5-methylisoxazol-3-yl)morpholine (300 mg, 1.43 mmol, 73.72% yield) was obtained as white gum. (M+H)⁺=169.2; purity=80% (220 nm). Retention time=0.115 min. 1H NMR (400 MHz, CDCl₃) δ=7.52-7.44 (m, 8H), 6.18 (s, 1H), 6.09-6.02 (m, 1H), 5.17 (dd, J=1.7, 10.9 Hz, 1H), 5.09 (br d, J=9.3 Hz, 1H), 4.40-4.10 (m, 8H), 3.85-3.49 (m, 4H), 3.16-2.92 (m, 3H), 2.43 (s, 2H), 2.34-2.27 (m, 4H), 1.32-1.24 (m, 3H).

Step 5: To a solution of 2-(3-methylisoxazol-5-yl)morpholine (1.00 eq, 100 mg, 0.595 mmol) and 2-chloro-4-(4-chloro-2-fluoro-phenyl)-6,7-dimethyl-pteridine (1.20 eq, 231 mg, 0.713 mmol) in DMSO (3 mL) was added DIEA (5.00 eq, 0.50 mL, 2.97 mmol), then the mixture was stirred at 100° C. for 2 h. LCMS (5-95AB/1.5 min): RT=0.975 min, 455.2=[M+H]⁺, ESI⁺ showed 94% of desired product. The reaction mixture was poured into H₂O (50 mL) and extracted with organic solvent (50 mL twice). The combined organic layers were washed with brine (50 mL), dried over Na₂SO₄ and concentrated in vacuo to give the residue. The residue was purified by prep-HPLC (Unisil 3-100 C18 Ultra 150*50 mm*3 um, water (FA)-ACN) and lyophilized to give 4-[4-(4-chloro-2-fluoro-phenyl)-6,7-dimethyl-pteridin-2-yl]-2-(3-methylisoxazol-5-yl)morpholine (6.1 mg, 0.0127 mmol, 2.14% yield) as yellow solid and 4-[4-(4-chloro-2-fluoro-phenyl)-6,7-dimethyl-pteridin-2-yl]-2-(5-methylisoxazol-3-yl)morpholine (50 mg, 0.104 mmol, 17.56% yield) as yellow solid. 1H NMR (400 MHz, CDCl₃) δ=7.70-7.64 (m, 1H), 7.34-7.28 (m, 3H), 6.18 (s, 1H), 4.83-4.75 (m, 2H), 4.16-4.08 (m, 1H), 3.89-3.80 (m, 1H), 3.56-3.45 (m, 2H), 2.75-2.71 (m, 3H), 2.61 (s, 3H), 2.32 (s, 3H), and 1H NMR (400 MHz, CDCl₃) δ=7.71-7.63 (m, 1H), 7.34-7.31 (m, 1H), 7.28 (d, J=1.9 Hz, 1H), 6.18 (s, 1H), 6.11 (d, J=0.6 Hz, 1H), 5.21-5.04 (m, 1H), 4.93-4.72 (m, 2H), 4.18-4.08 (m, 1H), 3.89-3.80 (m, 1H), 3.55-3.45 (m, 1H), 3.42-3.32 (m, 1H), 2.75-2.71 (m, 3H), 2.62-2.59 (m, 3H), 2.45 (s, 1H), 2.32 (s, 2H).

Step 6: The mixture was separated by SFC (Column: Chiralpak IC-3 50×4.6 mm I.D., 3 um; Mobile phase: Phase A for CO₂, and Phase B for IPA+ACN (0.05% DEA); Gradient elution: 40% IPA+ACN (0.05% DEA) in CO₂) and lyophilized to give 4-[4-(4-chloro-2-fluoro-phenyl)-6,7-dimethyl-pteridin-2-yl]-2-(3-methylisoxazol-5-yl)morpholine (17 mg, 0.0347 mmol, 157.70% yield) as yellow solid and 4-[4-(4-chloro-2-fluoro-phenyl)-6,7-dimethyl-pteridin-2-yl]-2-(5-methylisoxazol-3-yl)morpholine (6.2 mg, 0.0129 mmol, 58.52% yield) as yellow solid. 1H NMR (400 MHz, CDCl₃) δ=7.70-7.64 (m, 1H), 7.34-7.28 (m, 2H), 6.18 (s, 1H), 5.16-5.01 (m, 1H), 4.86-4.72 (m, 2H), 4.17-4.07 (m, 1H), 3.84 (dt, J=2.8, 11.2 Hz, 1H), 3.58-3.40 (m, 2H), 2.74 (s, 3H), 2.61 (s, 3H), 2.32 (s, 3H), 1.49-1.46 (m, 1H). LCMS: RT=0.980 min, 455.2=[M+H]⁺ 1H NMR (400 MHz, CDCl₃) δ=7.63 (s, 1H), 7.31 (br dd, J=2.0, 8.3 Hz, 2H), 6.11 (d, J=0.7 Hz, 1H), 5.22-5.11 (m, 1H), 4.96-4.72 (m, 2H), 4.20-4.11 (m, 1H), 3.92-3.75 (m, 1H), 3.46-3.31 (m, 2H), 2.73 (s, 3H), 2.60 (s, 3H), 2.45 (d, J=0.6 Hz, 3H). LCMS: RT=0.991 min, 455.2=[M+H]+

### Synthesis of Compound I-1254

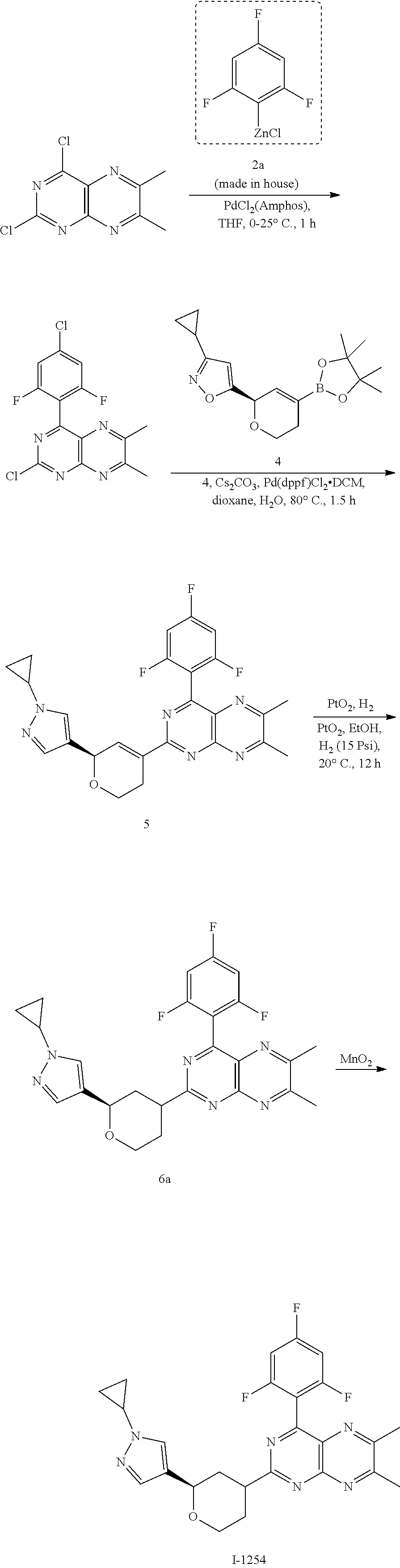

Step 1: A solution of 2,4-dichloro-6,7-dimethyl-pteridine (1.00 eq, 200 mg, 0.873 mmol) and PdCl₂ (amphos) (0.0500 eq, 31 mg, 0.0437 mmol) in THF (10 mL) under N₂ was cooled to 0° C., then chloro-(2,4,6-trifluorophenyl)zinc (1.20 eq, 7.5 mL, 1.05 mmol) was added dropwise at 0° C. The mixture was warmed to 25° C. and stirred for 1 h. The reaction solution was changed from pink to blue-black. LCMS showed the raw material was consumed most and the major peak showed desired MS (325.0[M+H]⁺; ESI⁺, LC-RT: 0.908 min). Water (30 mL) was added to the mixture followed by extraction with EtOAc (20 mL*2) and the organics backwashed with 10 ml saturated brine solution. The organics were then separated and dried (Na₂SO₄) before concentration to dryness. The crude was then purified by silica gel column (PE/EA=2/1,Rf=0.5) to give 2-chloro-6,7-dimethyl-4-(2,4,6-trifluorophenyl)pteridine (350 mg, 0.830 mmol, 95.07% yield) as a blue-black solid which was LCMS (77% purity, mixed with 22% di-Negishi BP). ¹H NMR (400 MHz, CDCl3) δ=6.90-6.83 (m, 2H), 2.88-2.85 (m, 3H), 2.75-2.72 (m, 3H). LCMS: (M+H)⁺=302.0.

Step 2: To a solution of 2-chloro-6,7-dimethyl-4-(2,4,6-trifluorophenyl)pteridine (1.00 eq, 200 mg, 0.616 mmol) and 1-cyclopropyl-4-[rac-(6R)-4-(4,4,5,5-tetramethyl-1,3,2-dioxaborolan-2-yl)-3,6-dihydro-2H-pyran-6-yl]pyrazole (1.10 eq, 214 mg, 0.678 mmol) and Cs₂CO₃ (1.00 eq, 200 mg, 0.616 mmol) in 1,4-Dioxane (7 mL) and Water (0.7000 mL), was added Pd(dppf)Cl₂ DCM (0.0500 eq, 25 mg, 0.0308 mmol), the mixture was stirred at 80° C. for 1.5 h. LCMS showed the desired product was formed. The mixture was concentrated to give a crude product. The residue was purified by preparative HPLC (column: Phenomenex Luna C18 150*25 mm*10 um; mobile phase: [water (FA)-ACN] B %: 46%-76%, 12 min) and lyophilized. 6,7-dimethyl-2-[rac-(6R)-6-(1-cyclopropylpyrazol-4-yl)-3, 6-dihydro-2H-pyran-4-yl]-4-(2, 4, 6-trifluorophenyl) pteridine (120 mg, 0.243 mmol, 39.49% yield) was obtained as gray solid. LCMS, 479.2 [M+H]⁺, ESI⁺

Step 3: To a solution of 2-[(6R)-6-(1-cyclopropylpyrazol-4-yl)-3,6-dihydro-2H-pyran-4-yl]-6,7-dimethyl-4-(2,4,6-trifluorophenyl)pteridine (1.00 eq, 110 mg, 0.230 mmol) in Ethanol (20 mL) was added PtO2 (0.930 eq, 49 mg, 0.214 mmol), the mixture was stirred at 20° C. for 12 h under H₂ (15 Psi). LCMS showed the starting material was consumed completely and desired product was formed. The mixture was filtered and filter liquor was concentrated to give a crude product. The crude product was used directly in the next step without further purification. LCMS: 485.1 [M+H]⁺, ESI⁺.

Step 4: To a solution of 2-[(2R)-2-(1-cyclopropylpyrazol-4-yl)tetrahydropyran-4-yl]-6,7-dimethyl-4-(2,4,6-trifluorophenyl)-5,6,7,8-tetrahydropteridine (1.00 eq, 120 mg, 0.248 mmol) in DCE (5 mL) was added MnO2 (20.0 eq, 431 mg, 4.95 mmol), the mixture was stirred at 20° C. for 12 h. The mixture was added MnO2 (20.0 eq, 431 mg, 4.95 mmol),The mixture was stirred at 20° C. for 12 h. LCMS showed the reaction was completed. The mixture was filtered and filter liquor was concentrated to give a crude product. The crude product was purified by preparative HPLC (column: Phenomenex Luna C18 75*30 mm*3 um; mobile phase: [water (FA)-ACN]; B %: 42%-72%, 7 min) and lyophilized. 2-[(2R)-2-(1-cyclopropylpyrazol-4-yl) tetrahydropyran-4-yl]-6, 7-dimethyl-4-(2, 4, 6-trifluorophenyl)pteridine (51 mg, 0.101 mmol, 40.98% yield) was obtained as yellow solid. SFC showed two peaks (ratio was 94:6). LCMS: 481.2 [M+H]⁺, ESI⁺, ¹H NMR (400 MHz, CDCl3) δ=7.49 (s, 2H), 6.85 (t, J=8.3 Hz, 2H), 4.54 (dd, J=1.6, 11.4 Hz, 1H), 4.30-4.20 (m, 1H), 3.87-3.73 (m, 1H), 3.54 (td, J=3.5, 7.3 Hz, 2H), 2.84 (s, 3H), 2.73-2.69 (m, 3H), 2.43 (br d, J=13.3 Hz, 1H), 2.27-2.13 (m, 3H), 1.15-1.03 (m, 2H), 1.01-0.89 (m, 2H).

### Synthesis of I-1260

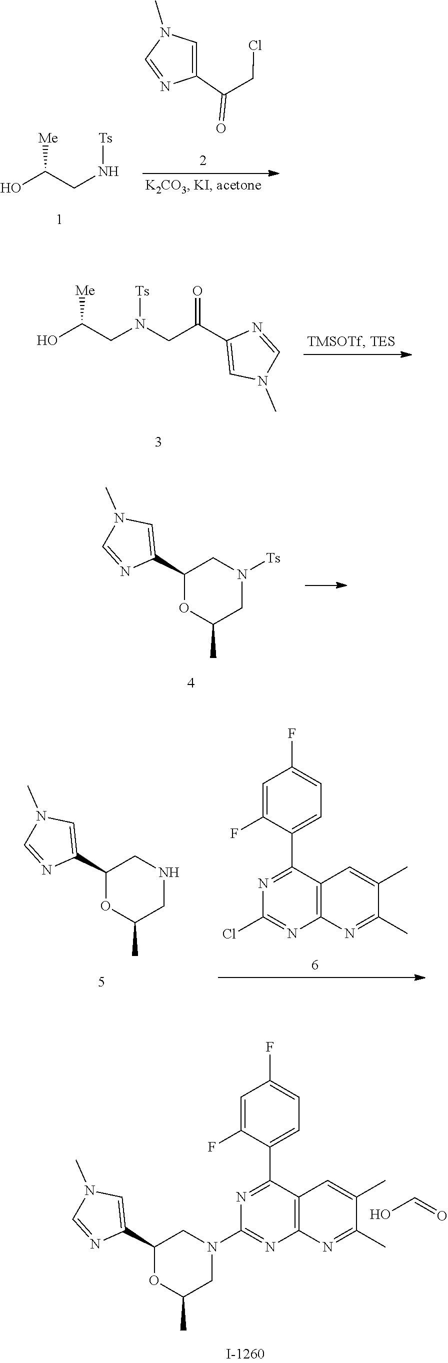

Step 1: To a yellow solution of N-[(2R)-2-hydroxypropyl]-4-methyl-benzenesulfonamide (1.00 eq, 1.00 g, 4.36 mmol) in Acetone (15 mL) was added 2-chloro-1-(1-methylimidazol-4-yl)ethanone (1.50 eq, 1037 mg, 6.54 mmol), K₂CO₃ (3.00 eq, 1808 mg, 13.1 mmol) and KI (1.00 eq, 724 mg, 4.36 mmol), to give a yellow suspension, the mixture was stirred at 30° C. for 12 h. The mixture was a red suspension. The mixture was filtered and the filtrate was concentrated in vacuo. N-[(2R)-2-hydroxypropyl]-4-methyl-N-[2-(1-methylimidazol-4-yl)-2-oxo-ethyl]benzenesulfonamide (1.40 g, 3.70 mmol, 84.95% yield) was obtained as yellow oil, LC-MS: Rt: 0.703 min; 334.0=[M+H−18]⁺, ESI⁺; 94.6% purity at 220 nm.

Step 2: To a yellow solution of N-[(2R)-2-hydroxypropyl]-4-methyl-N-[2-(1-methylimidazol-4-yl)-2-oxo-ethyl]benzenesulfonamide (1.00 eq, 1.40 g, 3.98 mmol) in DCM (20 mL) was added TES (10.0 eq, 13 mL, 39.8 mmol) and TMSOTf (10.0 eq, 7.2 mL, 39.8 mmol) at 0° C., to give yellow solution, the mixture was stirred at 20° C. for 12 h. The mixture was yellow solution. The reaction mixture was poured into NaHCO₃ solution (50 mL), the aqueous phase was extracted with DCM (50 mL* 3). The combined organic phase was washed with brine (50 mL*3), dried with anhydrous Na₂SO₄, filtered and concentrated to give a crude product in vacuum. The crude product was purified by column chromatography on silica gel eluted with EA (0-100%) in PE. (2R,6R)-2-methyl-6-(1-methylimidazol-4-yl)-4-(p-tolylsulfonyl)morpholine (100 mg, 0.268 mmol, 6.74% yield) was obtained as colorless oil, LC-MS: Rt: 0.16 min; 336.0=[M+H]⁺, ESI⁺; 100% purity at 220 nm. ¹H NMR (400 MHz, CDCl3) δ=7.65 (d, J=8.3 Hz, 2H), 7.41-7.30 (m, 3H), 6.86 (d, J=1.1 Hz, 1H), 4.65 (dd, J=2.6, 10.6 Hz, 1H), 3.94-3.76 (m, 2H), 3.69-3.59 (m, 4H), 2.59 (t, J=11.1 Hz, 1H), 2.44 (s, 3H), 2.18-2.08 (m, 1H), 1.20 (d, J=6.3 Hz, 3H).

Step 3: To a yellow solution of (2R,6R)-2-methyl-6-(1-methylimidazol-4-yl)-4-(p-tolylsulfonyl)morpholine (1.00 eq, 90 mg, 0.268 mmol) in Methanol (2 mL) was added Mg (Chips) (10.0 eq, 64 mg, 2.68 mmol) in N₂, to give a yellow suspension. The mixture was stirred at 80° C. for 12 h. The mixture was white suspension. The mixture was filtered and the filtrate was concentrated in vacuo. (2R,6R)-2-methyl-6-(1-methylimidazol-4-yl)morpholine (60 mg, 0.265 mmol, 98.71% yield) was obtained as white gum, LC-MS: Rt: 0.262 min; 182.1=[M+H]⁺, ESI⁺.

Step 4: To a white suspension of (2R,6R)-2-methyl-6-(1-methylimidazol-4-yl)morpholine (1.00 eq, 48 mg, 0.265 mmol) in DMSO (2 mL) was added 2-chloro-4-(2,4-difluorophenyl)-6,7-dimethyl-pyrido[2,3-d]pyrimidine (1.00 eq, 81 mg, 0.265 mmol) and DIPEA (4.00 eq, 0.18 mL, 1.06 mmol), to give a white suspension, the mixture was stirred at 100° C. for 1 h. The mixture was red solution. LCMS showed 22% starting material still remained and 34% desired mass was detected. The mixture was concentrated to give a crude product in vacuum. The crude product was purified by prep-HPLC (FA, column: Phenomenex Luna C18 150*25 mm*10 um; mobile phase: [water(FA)-ACN]; B %: 7%-37%, 10 min) and lyophilized. LCMS showed the product was impure. The crude product was re-purified by prep-HPLC (FA, column: Phenomenex Luna C18 150*25 mm*10 um; mobile phase: [water(FA)-ACN]; B %: 9%-39%, 10 min) and lyophilized. (2R,6R)-4-[4-(2,4-difluorophenyl)-6,7-dimethyl-pyrido[2,3-d]pyrimidin-2-yl]-2-methyl-6-(1-methylimidazol-4-yl)morpholine; formic acid (18 mg, 0.0302 mmol, 11.42% yield) was obtained as yellow solid, I-1260: LC-MS: Rt: 0.736 min; 451.2=[M+H]⁺, ESI⁺; 85.8% purity at 220 nm. 1H NMR (400 MHz, CDCl3) δ=7.83 (d, J=8.1 Hz, 1H), 7.68-7.55 (m, 2H), 7.17-6.97 (m, 3H), 5.31-5.19 (m, 1H), 5.13-4.96 (m, 1H), 4.80-4.66 (m, 1H), 3.87 (ddd, J=2.5, 6.4, 10.3 Hz, 1H), 3.77 (s, 3H), 3.22 (t, J=12.3 Hz, 1H), 2.87 (br dd, J=11.1, 13.3 Hz, 1H), 2.71 (s, 3H), 2.36 (s, 3H), 1.34 (d, J=6.2 Hz, 3H).

### Synthesis of Compounds I-1265

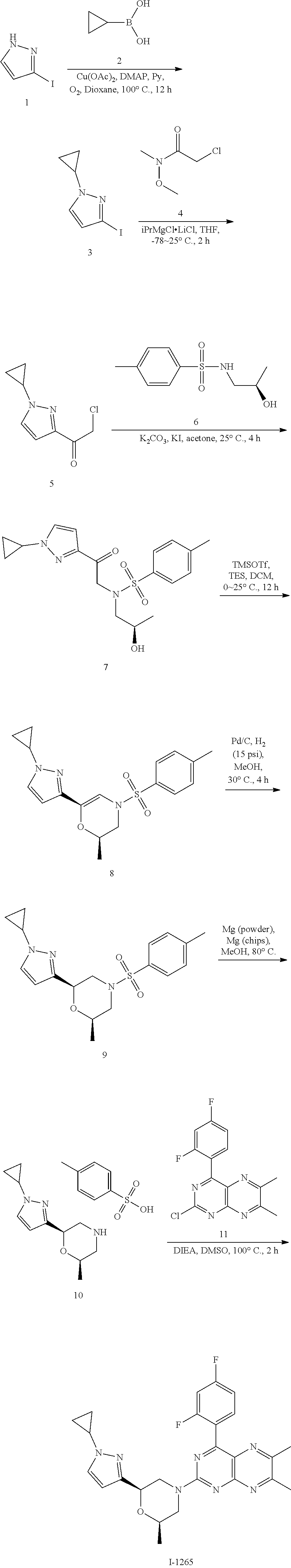

Step 1: To a solution of 4-iodo-1H-pyrazole (1.00 eq, 5.00 g, 25.8 mmol) in 1,4-Dioxane (150 mL) was added cyclopropylboronic acid (2.00 eq, 4.43 g, 51.6 mmol), Cu(OAc)₂ (1.00 eq, 4.68 g, 25.8 mmol), DMAP (4.00 eq, 12.58 g, 103 mmol) and pyridine (2.50 eq, 5.2 mL, 64.4 mmol). The resulting mixture was stirred at 100° C. for 16 hours under oxygen atmosphere (15 psi). Color of the solution was changed from blue to black. LCMS. Retention time=0.847 showed starting material was consumed completely and 94% desired mass was detected. The mixture was poured into water (100 mL) and was extracted with EtOAc (200 mL*3).The combined organic layers was washed with brine (100 mL), dried over Na₂SO₄ and concentrated to give the crude. The crude was purified by column chromatography on silica gel eluted with petroleum ether/ethyl acetate=100/1 to 1/1 (petroleum ether/ethyl acetate=5/1, the desired product R_{f}=0.3) to give 1-cyclopropyl-4-iodo-pyrazole (4.50 g, 18.3 mmol, 70.86% yield) as a light-yellow oil. (M+H)⁺=236.1; purity=64% (220 nm). Retention time=0.847 min. ¹H NMR (400 MHz, CDCl₃) δ=7.28 (d, J=2.4 Hz, 1H), 6.38 (d, J=2.3 Hz, 1H), 3.63-3.53 (m, 1H), 1.15-1.10 (m, 2H), 1.05-0.99 (m, 2H).

Step 2: To a solution of 1-cyclopropyl-3-iodo-pyrazole (1.00 eq, 1000 mg, 4.27 mmol) in THF (60 mL) was add isopropylmagnesium chloride (1.20 eq, 3.9 mL, 5.13 mmol) at −78° C. and stirred for 30 min, then 2-chloro-N-methoxy-N-methylacetamide (1.10 eq, 647 mg, 4.70 mmol) was added and stirred at 25° C. for 1 hour. LCMS (5-95AB/1.5 min): RT=0.663 min, 185.3=[M+H]⁺, ESI⁺ showed 80% of desired mass. The reaction mixture was poured into saturated aqueous NH₄Cl (100 mL) slowly and stirred at 0° C. for 10 min, then the mixture was extracted with EtOAc (100 mL*3). The organic layer was washed with brine, dried by Na₂SO₄. The solution was concentrated to give the residue. The residue was purified by flash silica gel chromatography on silica gel eluted with petroleum ether/ethyl acetate=100/1 to 1/1 (petroleum ether/ethyl acetate=5/1, the desired product R_{f}=0.3) to give 2-chloro-1-(1-cyclopropylpyrazol-3-yl) ethanone (1200 mg, 6.04 mmol, 40.42% yield) was obtained as colorless solid, LCMS: (M+H)⁺=185.2; purity=96% (220 nm). Retention time=0.668 min.

Step 3: To a solution of 2-chloro-1-(1-cyclopropylpyrazol-3-yl)ethanone (1.00 eq, 500 mg, 2.71 mmol) and 4-methyl-N-[(2R)-2-hydroxypropyl]benzenesulfonamide (1.20 eq, 745 mg, 3.25 mmol) in Acetone (12 mL) was added KI (1.00 eq, 450 mg, 2.71 mmol) and K₂CO₃ (3.00 eq, 1123 mg, 8.12 mmol), the mixture stirred at 25° C. for 2 hours. LCMS: RT=0.895 min) showed the starting material was consumed completely, and 45% desired mass was detected. The reaction mixture was poured into water (50 mL) and extracted by EtOAc (50*3 mL). The organic layer was washed with brine, dried by Na₂SO₄. The solution was concentrated to give the residue. The crude product was purified by column chromatography on silica gel eluted with petroleum ether/ethyl acetate=100/1 to 1/1, (petroleum ether/ethyl acetate=5/1, the desired product R_{f}=0.4) to give N-[(2R)-2-hydroxyN-[2-(1-cyclopropylpyrazol-3-yl)-2-oxo-ethyl]-4-methyl-N-[rac-(2R)-2-hydroxypropyl]benzenesulfonamid (200 mg, 0.477 mmol, 17.61% yield) was obtained as a colorless oil. (M+H)⁺=360.2; purity=85% (220 nm). Retention time=0.865 min.

Step 4: To a solution of N-[2-(1-cyclopropylpyrazol-3-yl)-2-oxo-ethyl]-N-[(2R)-2-hydroxypropyl]-4-methyl-benzenesulfonamide (1.00 eq, 200 mg, 0.530 mmol), TES (10.0 eq, 1.7 mL, 5.30 mmol) in DCM (2.5 mL), then TMSOTf (8.00 eq, 0.77 mL, 4.24 mmol) was added into the mixture at 0° C. The mixture was stirred at 30° C. for 12 hours. LCMS (5-95AB/1.5 min): RT=0.928 min, 360.2=[M+H]⁺, ESI⁺ showed 90% of desired product. The reaction mixture was poured into water (20 mL) and extracted by EtOAc (20*3 mL). The separated organic layer was washed with water, dried over Na₂SO₄ and evaporated to dryness. The crude product was purified by column chromatography on silica gel eluted with petroleum ether/ethyl acetate=100/1 to 1/1 (petroleum ether/ethyl acetate=3/1, the desired product R_{f}=0.4) to give (2R,6R)-2-(1-cyclopropylpyrazol-3-yl)-6-methyl-4-(p-tolylsulfonyl)morpholine (200 mg, 0.470 mmol, 88.76% yield) was obtained as colorless oil. (M+H)⁺=360.0; purity=90% (220 nm). Retention time=0.928 min. 1H NMR (400 MHz, CDCl₃) δ=7.67 (dd, J=8.3, 11.6 Hz, 4H), 7.36 (dd, J=2.3, 5.4 Hz, 3H), 7.33-7.29 (m, 3H), 6.74 (s, 1H), 6.28-6.25 (m, 1H), 6.17-6.13 (m, 1H), 4.77-4.68 (m, 1H), 3.90-3.76 (m, 3H), 3.67-3.49 (m, 5H), 2.98-2.91 (m, 1H), 2.53-2.39 (m, 8H), 2.19-2.10 (m, 1H), 1.31-1.16 (m, 10H), 1.15-1.05 (m, 5H), 1.05-0.87 (m, 1H).

Step 5: To a solution of (2R)-6-(1-cyclopropylpyrazol-3-yl)-2-methyl-4-(p-tolylsulfonyl)-2,3-dihydro-1,4-oxazine (1.00 eq, 200 mg, 0.556 mmol) in Methanol (10 mL) was added Pd/C (3.39 eq, 200 mg, 1.89 mmol) under N₂ atmosphere. The mixture was purged with H₂ 3 times, then the mixture was stirred at 30° C. for 12 hours under H₂ (15 psi). LCMS (5-95AB/1.5 min): RT=0.903 min, 362.2=[M+H]⁺, ESI⁺ showed 100% of desired product. The reaction mixture was filtered through celite, the filtrate was evaporated under reduced pressure to give the crude product. The crude product was used for the next step directly. (M+H)⁺=362.2; purity=100% (220 nm). Retention time=0.903 min.

Step 6: To a solution of (2R,6R)-2-(1-cyclopropylpyrazol-3-yl)-6-methyl-4-(p-tolylsulfonyl)morpholine (1.00 eq, 200 mg, 0.553 mmol) in Methanol (20 mL) was added Mg (powder) (9.79 eq, 130 mg, 5.42 mmol) and Mg (chips) (9.79 eq, 130 mg, 5.42 mmol) at 25° C. and then the mixture was stirred for 12 hours under N₂ at 80° C. LCMS showed 60% of desired product was detected and 30% of starting material was remained. The reaction mixture was filtered through celite, the filtrate was evaporated under reduced pressure to give the crude product, which was then dissolved into Methanol (20 mL), Mg (powder) (10.0 eq, 133 mg, 5.53 mmol) and Mg (chips) (10.0 eq, 133 mg, 5.53 mmol) were added to the solution, purged with N₂ for 3 times and stirred at 80° C. for another 12 hours under N₂. TLC indicated Reactant 1 was consumed completely and one new spot formed. (PE/EtOAc=3/1, starting material R_{f}=0.3; product R_{f}=0.5). The reaction mixture was filtered by celite and the filtrate was evaporated under reduced pressure to give the crude product. The crude product was used for the next step directly. (M+H)⁺=362.2; purity=33% (220 nm). Retention time=0.899 min.

Step 7: (2R,6R)-2-(1-cyclopropylpyrazol-3-yl)-4-[4-(2,4-difluorophenyl)-6,7-dimethyl-pteridin-2-yl]-6-methyl-morpholine. To a solution of (2R,6R)-2-(1-cyclopropylpyrazol-3-yl)-6-methyl-morpholine (1.00 eq, 210 mg, 1.01 mmol) and 2-chloro-4-(2,4-difluorophenyl)-6,7-dimethyl-pteridine (1.20 eq, 373 mg, 1.22 mmol) in DMSO (0.5000 mL) was added DIEA (5.00 eq, 23 mg, 0.178 mmol), then the mixture was stirred at 100° C. for 2 hours. LCMS (5-95AB/1.5 min): RT=0.980 min, 478.3=[M+H]⁺, ESI⁺ showed 70% of desired product. The reaction mixture was poured into H₂O (50 mL) and extracted with organic solvent (50 mL twice). The combined organic layers were washed with brine (50 mL), dried over Na₂SO₄ and concentrated in vacuo to give the residue. The residue was purified by prep-HPLC (Unisil 3-100 C18 Ultra 150*50 mm*3 um water (FA)-ACN) to afford as a yellow solid. LCMS: RT=0.982 min, 478.2=[M+H]⁺ 1H NMR: (400 MHz, CDCl₃) δ=7.71 (q, J=7.2 Hz, 1H), 7.41 (d, J=2.3 Hz, 1H), 7.08-6.92 (m, 2H), 6.30 (d, J=2.2 Hz, 1H), 5.21-4.92 (m, 2H), 4.72 (dd, J=2.6, 11.1 Hz, 1H), 3.93-3.82 (m, 1H), 3.63-3.53 (m, 1H), 3.33-3.23 (m, 1H), 2.95-2.85 (m, 1H), 2.71 (s, 3H), 2.59 (s, 3H), 1.35 (d, J=6.1 Hz, 3H), 1.14-1.08 (m, 2H), 1.05-0.99 (m, 2H).

### Synthesis of Compounds I-1273

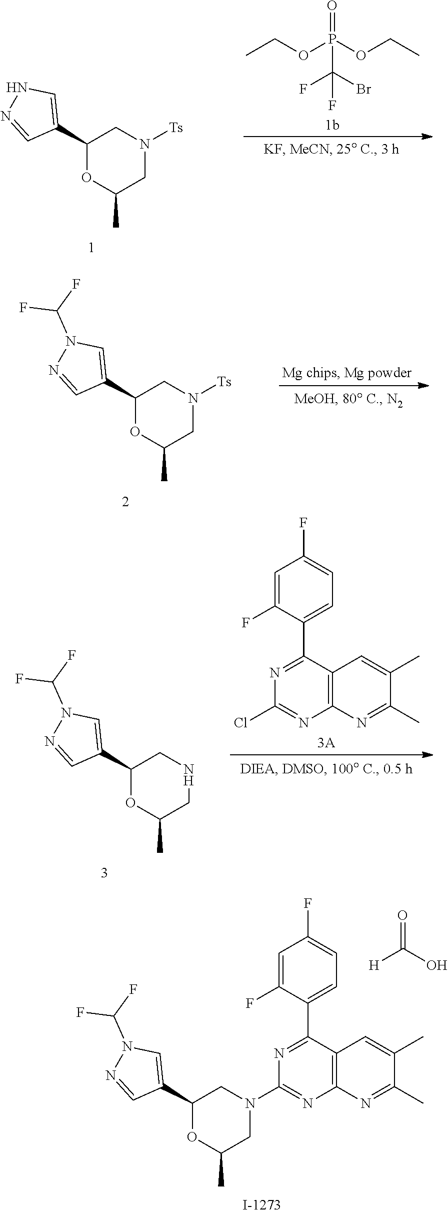

Step 1: To a solution of (2R,6S)-2-methyl-4-(p-tolylsulfonyl)-6-(1H-pyrazol-4-yl)morpholine (1.00 eq, 130 mg, 0.404 mmol), KF (2.00 eq, 47 mg, 0.809 mmol) and 1-[[bromo (difluoro) methyl]-ethoxy-phosphoryl]oxyethane (1.50 eq, 162 mg, 0.607 mmol) in MeCN (5 mL) then the mixture was stirred at 40° C. for 12 h. LCMS showed the starting material was consumed completely and a major peak with mass (97%, MS: 372.1 [M+H]⁺, ESI pos). The reaction mixture was partitioned between EtOAc (40 mL) and water (40 mL). The separated organic layer was washed with water, dried over Na₂SO₄ and evaporated to dryness. The reaction mixture was filtered and concentrated under reduced pressure to give a residue. The residue was purified by prep-TLC (SiO₂, PE:EtOAc=1:1; R_{f}=0.4) to afford (2S,6R)-2-[1-(difluoromethyl)pyrazol-4-yl]-6-methyl-4-(p-tolylsulfonyl) morpholine 2 (150 mg, 0.404 mmol, 99.85% yield) as white solid. LCMS (M+H)⁺=372.1; purity=98% (220 nm). Retention time=0.892 min. ¹H NMR (400 MHz, DMSO-d6) δ ppm 1.10 (d, J=6.24 Hz, 3H) 2.41 (s, 3H) 3.58 (br d, J=11.25 Hz, 1H) 3.70 (br d, J=11.37 Hz, 1H) 3.77 (ddd, J=10.15, 6.30, 2.38 Hz, 1H) 4.30-4.35 (m, 2H) 4.66 (dd, J=10.45, 2.38 Hz, 1H) 7.46 (d, J=8.07 Hz, 2H) 7.59 (s, 1H) 7.66 (d, J=8.31 Hz, 2H) 7.74 (s, 1H) 7.78 (s, 1H) 7.89 (s, 1H) 8.22 (s, 1H).

Step 2: To a solution of (2S,6R)-2-[1-(difluoromethyl)pyrazol-4-yl]-6-methyl-4-(p-tolylsulfonyl)morpholine (1.00 eq, 150 mg, 0.404 mmol) in Methanol (10 mL) was added Mg (powder) (10.0 eq, 97 mg, 4.04 mmol) and Mg (chips) (10.0 eq, 97 mg, 4.04 mmol) at 25° C. and then the mixture was stirred at 80° C. for 12 h under N₂. LCMS showed the starting material was consumed completely but only a trace amount of desired compound was detected. (MS: 218.1 [M+H]⁺, ESI pos). The reaction mixture was filtered by celite to afford crude product as white solid. LCMS (M+H)⁺=218.1; purity=2% (220 nm).

Step 3: To a solution of (2S,6R)-2-[1-(difluoromethyl)pyrazol-4-yl]-6-methyl-morpholine (1.00 eq, 200 mg, 0.921 mmol) and 2-chloro-4-(2,4-difluorophenyl)-6,7-dimethyl-pyrido[2,3-d]pyrimidine (1.00 eq, 281 mg, 0.921 mmol) in DMSO (3 mL) was added DIEA (4.00 eq, 476 mg, 3.68 mmol). The mixture was stirred at 100° C. for 0.5 hour. LCMS (product: RT=0.826 min; m/z: 487.2 [M+H]⁺. 10% purity at 220 nm) showed the starting material was consumed completely and desire MS was detected. The reaction was filtered and the filtrate was purified by prep-HPLC (flow: 25 mL/min; gradient: from 27-57% water (0.1% FA)-ACN over 7 min; column: Unisil 3-100 C18 Ultra 150*50 mm*3 um) and lyophilized to afford (2S,6R)-2-[1-(difluoromethyl)pyrazol-4-yl]-4-[4-(2,4-difluorophenyl)-6,7-dimethyl-pyrido[2,3-d]pyrimidin-2-yl]-6-methyl-morpholine; formic acid (8.5 mg, 0.0145 mmol, 1.58% yield) as yellow solid. LCMS (M+H)⁺=487.2; purity=100% (220 nm). Retention time=0.826 min. HPLC: Retention time: 1.848 min, 90.857% purity at 220 nm. 1H NMR (400 MHz, DMSO-d6) δ ppm 1.24 (br s, 3H) 2.30 (s, 3H) 2.57 (s, 3H) 2.72-2.84 (m, 1H) 2.99-3.12 (m, 1H) 3.73-3.84 (m, 1H) 4.65 (br d, J=10.88 Hz, 1H) 4.71-4.92 (m, 2H) 7.28-7.37 (m, 1H) 7.46-7.53 (m, 1H) 7.54-7.63 (m, 2H) 7.65 (br s, 1H) 7.68-7.76 (m, 1H) 7.79 (s, 1H) 7.89 (br s, 1H) 7.94 (s, 1H).

### Synthesis of I-1283

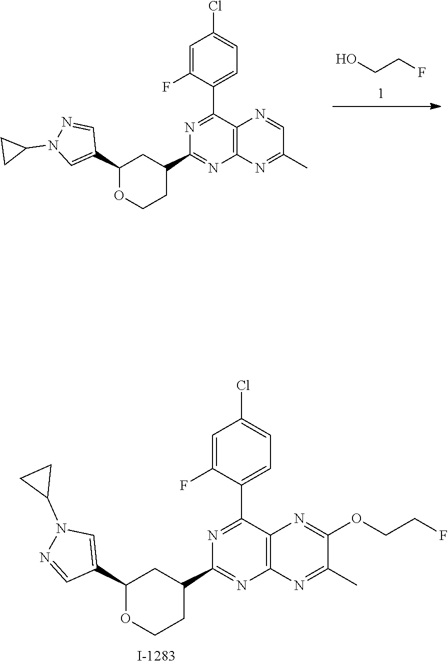

To a yellow solution of 4-(4-chloro-2-fluoro-phenyl)-2-[(2R,4S)-2-(1-cyclopropylpyrazol-4-yl)tetrahydropyran-4-yl]-7-methyl-pteridine (1.00 eq, 100 mg, 0.215 mmol) in 2-FLUOROETHANOL (160 eq, 2.0 mL, 34.3 mmol) was added CAN (1.50 eq, 177 mg, 0.323 mmol), to give a red solution, the mixture was stirred at 20° C. for 2 h. The mixture was yellow solid. LCMS showed 31% starting material still remained and 45% desired mass was detected. The reaction mixture was poured into Na₂SO₃ solution (20 mL) and EA (20 mL), the mixture was filtered and the filtrate was extracted with EA (20 mL*3). The combined organic phase was washed with brine (20 mL*3), dried with anhydrous Na₂SO₄, filtered and concentrated to give a crude product in vacuum. The crude product was purified by prep-HPLC (FA, column: Phenomenex Luna C18 150*25 mm*10 um; mobile phase: [water (FA)-ACN]; B %: 52%-82%, 10 min) and lyophilized. 4-(4-chloro-2-fluoro-phenyl)-2-[(2R,4S)-2-(1-cyclopropylpyrazol-4-yl)tetrahydropyran-4-yl]-6-(2-fluoroethoxy)-7-methyl-pteridine (7.7 mg, 0.0145 mmol, 6.75% yield) was obtained as yellow solid. I-1283: LC-MS: Rt: 0.906 min; 527.2=[M+H]+, ESI+; 99.4% purity at 220 nm. 1H NMR (400 MHz, CDCl3) δ=7.69 (t, J=7.8 Hz, 1H), 7.50 (d, J=1.5 Hz, 2H), 7.35 (dd, J=1.9, 8.3 Hz, 1H), 7.29 (d, J=1.9 Hz, 1H), 4.92-4.83 (m, 1H), 4.79-4.70 (m, 1H), 4.68-4.52 (m, 3H), 4.26 (d, J=10.8 Hz, 1H), 3.88-3.70 (m, 1H), 3.62-3.46 (m, 2H), 2.81 (s, 3H), 2.42 (d, J=13.3 Hz, 1H), 2.26-2.15 (m, 3H), 1.14-1.06 (m, 2H), 1.03-0.94 (m, 2H).

### Synthesis of Compounds I-1288 and I-1293

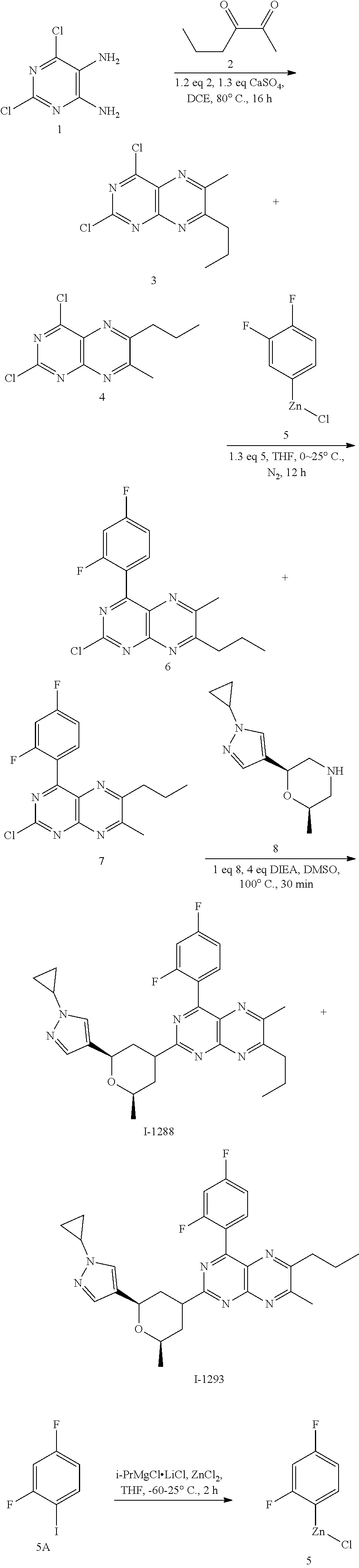

Step 1:To a solution of hexane-2,3-dione (1.20 eq, 765 mg, 6.70 mmol) in DCE (100 mL) was added CaSO₄ (1.30 eq, 989 mg, 7.26 mmol) followed by 2,6-dichloropyrimidine-4,5-diamine (1.00 eq, 1000 mg, 5.59 mmol). The reaction mixture was stirred at 80° C. for 16 hr. LCMS (5-95AB/1.5 min): RT=0.895 min, 257.1=[M+H]⁺, ESI⁺ showed desired product MS. The reaction was diluted with EtOAc (200 mL) and then filtered through a pad of celite. The filter cake was washed with MeOH (100 mL) and EtOAc (150 mL) and DCM (150 mL). The combined filtrate was concentrated under reduced pressure to afford a residue. The residue was purified by flash chromatography on silica gel eluting with PE/EtOAc (9:1 to 8:1 to 1:1) to afford product as yellow solid, which LCMS (5-95AB/1.5 min): RT=0.853 min, 257.1=[M+H]⁺, ESI⁺ showed 100% of crude product, product as yellow oil, which LCMS (5-95AB/1.5 min): RT=0.862 min, 257.1=[M+H]⁺, ESI⁺ showed 100% of crude product and recycled starting material (870 mg) as yellow solid. The crude product was further purified by prep-TLC (PE: EtOAc=3:1, R_{f}=0.55) to afford 2,4-dichloro-6-methyl-7-propyl-pteridine (170 mg, 0.661 mmol, 11.84% yield) as yellow solid. LCMS: Rt: 0.875 min; [M+H]⁺=257.1; 96.9% purity at 220 nm. The crude product was further purified by prep-TLC (PE:EtOAc=3:1, R_{f}=0.55) to afford 2,4-dichloro-7-methyl-6-propyl-pteridine (150 mg, 0.583 mmol, 10.44% yield) as yellow oil. LCMS: Rt: 0.889 min; [M+H]⁺=257.1; 99.2% purity at 220 nm.

Step 2: A three necked bottle was equipped with 4-chloro-2-fluoro-1-iodo-benzene (1.00 eq, 1000 mg, 3.90 mmol), the bottle was sealed and purged with N₂ for 3 times, THF (10 mL) was added and the solution was cooled to −40° C. with stirring, iPrMgCl·LiCl (1.3 M in THF) (1.10 eq, 3.3 mL, 4.29 mmol) was added dropwise at −40° C. and the mixture was stirred for 30 min at this temperature. The reaction mixture was further cooled to −60° C. and ZnCl₂ (0.5 M in THF) (1.00 eq, 7.8 mL, 3.90 mmol) was added dropwise, the reaction solution turned into white floc, the reaction mixture was allowed to warm to room temperature gradually and stirred for 1 hr. the white floc turned into colorless solution, chloro-(4-chloro-2-fluoro-phenyl)zinc (898 mg, 3.90 mmol, 99.96% yield) was obtained as colorless solution in THF. The crude product was used to next step without purification.

Step 3: A sealed bottle under a N₂ atmosphere was charged with 2,4-dichloro-6-methyl-7-propyl-pteridine (1.00 eq, 170 mg, 0.661 mmol) and PdCl₂(Amphos) (0.100 eq, 47 mg, 0.0661 mmol) and THF (6 mL) and purged with N₂ three times, then cooled to 0° C. Chloro-(2,4-difluorophenyl)zinc (1.10 eq, 156 mg, 0.727 mmol) was added dropwise to the reaction solution at 0° C., then the mixture was warmed to 25° C. and stirred for 12 hrs. LCMS (5-95AB/1.5 min): RT=0.977 min, 335.1=[M+H]⁺, ESI⁺ showed 50.7% of desired product. The reaction mixture was combined with to work up. The combined reaction was diluted with water (50 mL) and then extracted with ethyl acetate (50 mL*3). The combined organic layers were washed with brine, dried over Na₂SO₄, filtered and concentrated under reduced pressure to give a residue. The residue was purified by flash chromatography on silica gel eluting with PE/EtOAc (5:1) (TLC, PE:EtOAc, R_{f}=0.45) to afford 2-chloro-4-(2,4-difluorophenyl)-7-methyl-6-propyl-pteridine (80 mg, 0.108 mmol, 16.27% yield) as yellow oil and 2-chloro-4-(2,4-difluorophenyl)-6-methyl-7-propyl-pteridine (140 mg, 0.188 mmol, 28.46% yield) as yellow oil. The two product was combined. LCMS: Rt: 0.960 min; [M+H]⁺=335.1; 45% purity at 220 nm.

Step 4: To a solution of 2-chloro-4-(2,4-difluorophenyl)-6-methyl-7-propyl-pteridine (1.00 eq, 80 mg, 0.108 mmol), 2-chloro-4-(2,4-difluorophenyl)-7-methyl-6-propyl-pteridine (1.75 eq, 140 mg, 0.188 mmol) and DIEA (4.00 eq, 56 mg, 0.430 mmol) in DMSO (10 mL) was added (2S,6R)-2-(1-cyclopropylpyrazol-4-yl)-6-methyl-morpholine (1.20 eq, 67 mg, 0.129 mmol) at 25° C. Then the reaction mixture was stirred at 100° C. for 30 min. LCMS (5-95AB/1.5 min): RT=1.083 min, 506.3=[M+H]⁺, ESI⁺ showed 57% of desired product. The reaction mixture was combined with for further purification. The combined reaction mixture was diluted with water (50 mL) and then extracted with ethyl acetate (50 mL*3). The combined organic layers were washed with brine, dried over Na₂SO₄, filtered and concentrated under reduced pressure to give a residue. The residue was purified by prep-HPLC (Column, [Unisil 3-100 C18 Ultra 150*50 mm*3 um]; mobile phase: [ACN] and [H₂O] (conditions: [water (0.225% FA)-ACN], B %: 55%-85%; Detector, UV 254 nm. RT: [7 min]) to afford a mixture of two product (40 mg) as yellow solid, LCMS (5-95AB/1.5 min): RT=0.770 min, 506.1=[M+H]⁺, ESI⁺ showed 100% of the mixture products. The mixture of products was separated by SFC (Column: DAICEL CHIRALPAK IC (250 mm*30 mm, 10 um); Mobile phase: Phase A for CO₂, and Phase B for MeOH+ACN (0.05% DEA); Gradient elution: 40% MeOH+CAN (0.05% DEA) in CO₂ Flow rate: 3 mL/min; Detector: PDA Column Temp: 35C; Back Pressure: 100 Bar) to give two products. (2S,6R)-2-(1-cyclopropylpyrazol-4-yl)-4-[4-(2,4-difluorophenyl)-6-methyl-7-propyl-pteridin-2-yl]-6-methyl-morpholine (8.1 mg, 0.0155 mmol, 14.44% yield) (peak 1 in SFC) was afforded as yellow solid. (2S,6R)-2-(1-cyclopropylpyrazol-4-yl)-4-[4-(2,4-difluorophenyl)-7-methyl-6-propyl-pteridin-2-yl]-6-methyl-morpholine (14 mg, 0.0275 mmol, 25.53% yield) (peak 2 in SFC) was afforded as yellow solid. I-1288, LCMS: Rt: 740 min; [M+H]⁺=506.3; 97.3% purity at 220 nm and ¹H NMR (400 MHz, CDCl3) δ ppm 0.98-1.16 (m, 7H) 1.33 (d, J=6.13 Hz, 3H) 1.86 (dq, J=15.21, 7.44 Hz, 2H) 2.62 (s, 3H) 2.84 (br dd, J=12.94, 11.07 Hz, 1H) 2.90-3.00 (m, 2H) 3.08 (dd, J=13.13, 11.26 Hz, 1H) 3.58 (tt, J=7.00, 3.63 Hz, 1H) 3.77-3.89 (m, 1H) 4.61 (brd, J=9.13 Hz, 1H) 4.86-5.24 (m, 2H) 6.92-7.09 (m, 2H) 7.55 (brd, J=3.63 Hz, 2H) 7.71 (q, J=7.75 Hz, 1H); LCMS: Rt: 0.736 min; [M+H]⁺=506.3; 100% purity at 220 nm and ¹H NMR (400 MHz, CDCl3) δ ppm 0.95-1.05 (m, 5H) 1.08-1.15 (m, 2H) 1.33 (d, J=6.25 Hz, 3H) 1.76 (sxt, J=7.40 Hz, 2H) 2.73 (s, 3H) 2.80-2.88 (m, 3H) 3.08 (dd, J=13.20, 11.07 Hz, 1H) 3.58 (tt, J=7.25, 3.69 Hz, 1H) 3.83 (ddd, J=10.22, 6.35, 2.44 Hz, 1H) 4.60 (dd, J=10.76, 2.00 Hz, 1H) 4.90-5.19 (m, 2H) 6.93-7.00 (m, 1H) 7.04 (br t, J=8.13 Hz, 1H) 7.54 (d, J=3.75 Hz, 2H) 7.72 (q, J=7.71 Hz, 1H).

### Synthesis of I-1298

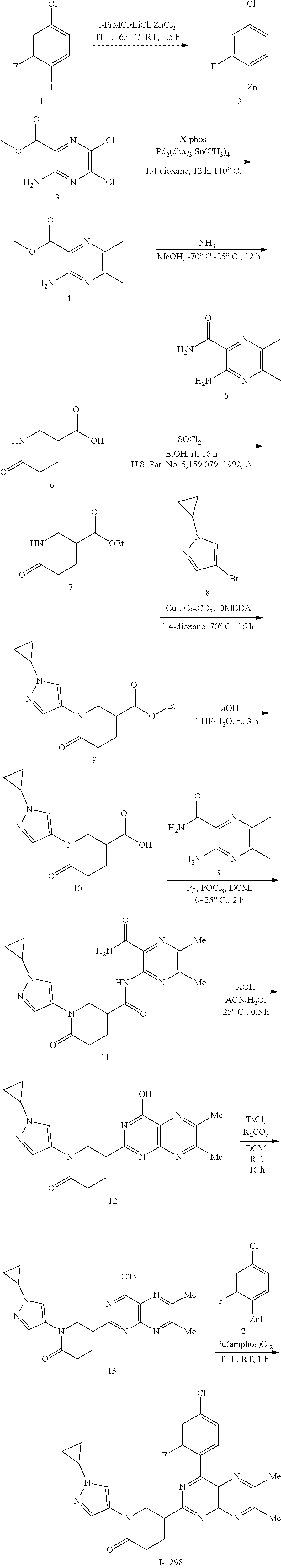

Step 1: To a mixture of methyl 3-amino-5,6-dichloro-pyrazine-2-carboxylate (4 g, 18.0 mmol, 1.0 eq) and tetramethylstannane (8.05 g, 45.0 mmol, 2.5 eq) in 1,4-dioxane (50 mL) were added X-phos (3.43 g, 7.2 mmol, 0.4 eq) and Pd₂(dba)₃ (1.03 g, 1.8 mmol, 0.1 eq). The mixture was degassed and stirred under N₂, then warmed up to 110° C. and stirred overnight. LCMS showed the main peak was the desired product and no starting material remained. The mixture was cooled to room temperature, diluted with water (50 mL), and extracted with CH₂Cl₂ (100 mL×3). The combined organics were washed with NaHCO₃ (aq) (100 mL) and brine (100 mL), dried over Na₂SO₄, and concentrated in vacuo to afford the crude product. The crude product was purified by FCC (Petroleum ether:EtOAc=52:48) to afford the desired product (3 g, 87.3%) as a yellow solid. LCMS: M+H=182, Ref time=0.85

Step 2: In a sealed tube, methyl 3-amino-5,6-dimethyl-pyrazine-2-carboxylate (300 mg, 1.66 mmol) was added to the solution of NH₃ in MeOH (2 mL, 7 mol/L)). The mixture was warmed up to 80° C. and stirred overnight. The reaction was concentrated and the residue was washed with MTBE (50 mL). The solid was dried in vacuum to afford the desired product (295 mg, 96.5%). LCMS: M+H=167.2, Ref time=0.572 min

Step 3: To a mixture of 6-oxopiperidine-3-carboxylic acid (500 mg, 3.49 mmol, 1.0 eq) in EtOH (3 mL) was added thionyl chloride (5.24 mmol, 0.38 mL, 1.5 eq) dropwise. The mixture was stirred at room temperature overnight. The mixture was concentrated in vacuum to afford the crude product. After washed with EtOAc (2 mL), the solid was collected. The solid was dried in vacuum to afford the desired product (480 mg, 72.2%). LCMS: M+H=172.2, Ref time=0.514. 1H NMR (400 MHz, DMSO) δ 10.28 (s, 1H), 4.13-4.07 (m, 2H), 3.34-3.23 (m, 2H), 2.83-2.77 (m, 1H), 2.27-2.12 (m, 2H), 2.02-1.95 (m, 1H), 1.88-1.79 (m, 1H), 1.21-1.17 (t, J=6.8 Hz, J=14 Hz, 3H)

Step 4: To a mixture of ethyl 6-oxopiperidine-3-carboxylate (300 mg, 1.75 mmol, 95% purity, 1.0 eq), CuI (166.9 mg, 0.88 mmol, 0.53 eq) and Cs₂CO₃ (1.08 g, 3.33 mmol, 2.0 eq) in 1,4-dioxane (5 mL) were added 4-bromo-1-cyclopropyl-pyrazole (426 mg, 2.28 mmol, 1.4 eq) and DMEDA (155 mg, 1.75 mmol, 1.05 eq). The mixture was degassed for 3 times and stirred under N₂. The mixture was warmed up to 70° C. overnight. The reaction was diluted with water (100 mL) and extracted with EtOAc (100 mL×3). The organic was combined and washed with NaHCO₃ (aq) (100 mL), dried over Na₂SO₄, and concentrated in vacuum to afford the crude product. The crude product was purified by FCC (Petroleum ether:EtOAc=72:28) to afford the desired product (180 mg, 35.2%) as a yellow oil. M+H=278 Ref time=0.98 min. 1H NMR (400 MHz, CDCl₃) δ 8.04 (s, 1H), 7.51 (s, 1H), 4.23-4.18 (dd, J=6.8, J=14, 2H), 3.90-3.80 (m, 2H), 3.60-3.55 (m, 1H), 2.96-2.91 (m, 1H), 2.67-2.51 (m, 2H), 2.23-2.18 (m, 1H), 2.11-2.03 (m, 1H), 1.30-1.25 (t, J=7.2, J=14, 3H), 1.14-1.10 (m, 2H), 1.02-0.97 (m, 2H).

Step 5: To a solution of ethyl 1-(1-cyclopropylpyrazol-4-yl)-6-oxo-piperidine-3-carboxylate (180 mg, 0.65 mmol, 1.0 eq) in THF (2 mL) was added the solution of 2M LiOH (2 mL, 3.24 mmol, 5.0 eq). The reaction was stirred at room temperature for 3 hours. The reaction was adjusted pH=4 with 1M HCl and freeze-dried. The residue was extracted with CH₂Cl₂ (100 mL×3). The organic was combined and concentrated in vacuum to afford the crude product (280 mg, 86.5%) as a yellow oil. LCMS: M+H=250 Ref time=0.552

Step 6: To a mixture of 1-(1-cyclopropylpyrazol-4-yl)-6-oxo-piperidine-3-carboxylic acid (500 mg, 2.0 mmol, 1.0 eq) in CH₂Cl₂ (8 mL) were added 3-amino-5,6-dimethyl-pyrazine-2-carboxamide (366 mg, 2.2 mmol, 1.1 eq) and pyridine (793 mg, 10.03 mmol, 5.0 eq). The mixture was stirred and cooled to 0° C., and then POCl₃ (615 mg, 4.0 mmol, 2.0 eq) in DCM (8 mL) was added dropwise. After completion, the mixture was warmed up to 25° C., and stirred for 3 hours. The mixture was quenched with CH₂Cl₂ (100 mL) and washed with 0.5M HCl (30 mL). The organic was washed with NaHCO₃ (aq) (50 mL) and brine (50 mL), dried over Na₂SO₄, concentrated in vacuum to afford the crude product (780 mg, 39.1%). The crude was subjected to next step without purification.

Step 7: To a mixture of 3-[[1-(1-cyclopropylpyrazol-4-yl)-6-oxo-piperidine-3-carbonyl]amino]-5,6-dimethyl-pyrazine-2-carboxamide (1 g, 2.52 mmol, 1.0 eq) in MeCN (5 mL) was added a solution of KOH (181 mg, 7.54 mmol, 3.0 eq) in water (15 mL). The mixture was stirred at room temperature for 1 hour. The mixture was adjusted pH=2 with 1M HCl and extracted with DCM (50 mL×3). The organic was washed with NaHCO₃ (aq) (50 mL) and brine (50 mL). The combined organics were dried over Na₂SO₄ and concentrated in vacuum. The crude product was purified by FCC (CH₂Cl₂: MeOH=100-90%) to afford the desired product (180 mg).

Step 8: To a solution of compound 12 (100 mg, 0.26 mmol, 1.00 eq) and TsCl (65 mg, 0.34 mmol, 1.3 eq) in DCM (10 mL) were added Et₃N (79 mg, 0.78 mmol, 3 eq). The reaction mixture was stirred at 25° C. under N₂ for 0.5 hour. LC-MS showed 2-(1-(1-cyclopropyl-1H-pyrazol-4-yl)-6-oxopiperidin-3-yl)-6,7-dimethylpteridin-4-yl 4-methylbenzenesulfonate was consumed and one main peak with desired m/z was detected. The mixture was evaporated. The crude was subjected to next step without further purification.

Step 9: To a round-bottomed flask was added 4-chloro-2-fluoro-1-iodobenzene (1.0 g, 3.1 mmol, 1.00 eq) in THF (12 mL) was added i-PrMgCl (2.00 M, 2.2 mL, 1.13 eq) dropwise at −40° C. The mixture was stirred at the same temperature for 30 minutes. Then the reaction mixture was cooled to −78° C., and ZnCl₂ (2.00 M, 2 mL, 1.03 eq) was added dropwise and the reaction mixture was allowed to warm to 20° C. for 1 hour, and a white turbid liquid formed. The crude product was used directly for next reaction.

Step 10: (80 mg, 0.15 mmol, 1 eq) and Pd(amphos)Cl₂ (6 mg, 0.008 mmol, 0.05 eq) in THF (5 mL) was added a solution of Compound 2 (242 mg, 0.75 mmol, 5 eq) in THF (5 mL). The mixture was stirred at room temperature for 3 hours. LC-MS showed 2-(1-(1-cyclopropyl-1H-pyrazol-4-yl)-6-oxopiperidin-3-yl)-6,7-dimethylpteridin-4-yl 4-methylbenzenesulfonate was consumed and one main peak with desired m/z was detected. The mixture was filtered and evaporated and purified by prep-HPLC (ACN-H₂O (0.1% FA); gradient: 5-95) and lyophilized to afford 5-(4-(4-chloro-2-fluorophenyl)-6,7-dimethylpteridin-2-yl)-1-(1-cyclopropyl-1H-pyrazol-4-yl)piperidin-2-one as a yellow solid (4.15 mg, 5.6%). LCMS: (M+H)⁺=592.3. Retention time=1.135 min. HPLC: purity=96.809% (254 nm); purity=97.767% (214 nm). Retention time=3.269 min. ¹H NMR (400 MHz, DMSO) δ 8.06 (s, 1H), 7.78 (t, J=8.0 Hz, 1H), 7.70 (dd, J=9.8, 2.0 Hz, 1H), 7.66 (s, 1H), 7.54 (dd, J=8.4, 2.0 Hz, 1H), 4.14-4.06 (m, 2H), 3.83-3.79 (m, 1H), 3.72-3.68 (m, 1H), 2.79 (s, 3H), 2.67 (s, 3H), 2.62 (dd, J=10.0, 6.8 Hz, 1H), 2.53 (d, J=5.4 Hz, 1H), 2.39-2.28 (m, 2H), 1.03-0.92 (m, 4H).

### Synthesis of Compounds I-1301 and I-1328

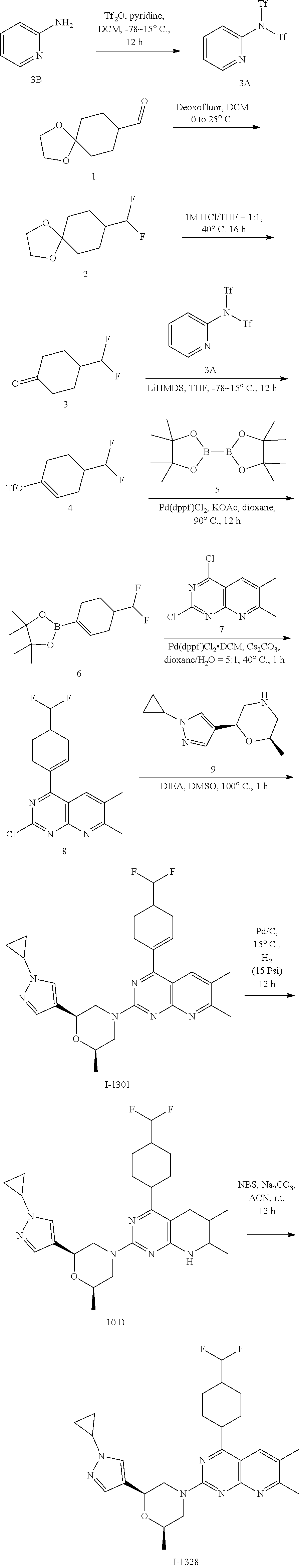

Step 1: To a colorless mixture of 2-AMINOPYRIDINE (1.00 eq, 5.00 g, 53.1 mmol), pyridine (2.10 eq, 9.0 mL, 112 mmol) in DCM (90 mL) under N₂ atmosphere. The reaction mixture was cooled to −78° C. and a solution of Triflic anhydride (2.10 eq, 31.48 g, 112 mmol) in DCM (10 mL) was added dropwise via a cannula with vigorous stirring. After the solution was stirred for 2 hr at −78° C., the cooling bath was removed and stirring was continued at 15° C. for 12 hr to give a yellow solution. LCMS showed the starting material was consumed completely and 46% desired MS (358.7[M+1]⁺, ESI pos) was found. The mixture was poured into cold water (60 mL) and extracted by DCM (3×60 mL), the combined organic layers were washed with brine, dried over anhydrous sodium sulfate, filtered and concentrated in vacuo. The crude material was purified by column on silica (20 g SiO₂ cartridge, PE:EA=10%, detection at 254 nm) and concentrated under reduced pressure to give white solid. 1,1,1-trifluoro-N-(2-pyridyl)-N-(trifluoromethylsulfonyl)methanesulfonamide (10.15 g, 26.9 mmol, 50.66% yield) was obtained as white solid. ¹H NMR (400 MHz, CDCl3) δ=8.66 (dd, J=1.4, 4.8 Hz, 1H), 7.96 (dt, J=1.9, 7.8 Hz, 1H), 7.56 (ddd, J=0.8, 4.8, 7.6 Hz, 1H), 7.48 (d, J=7.9 Hz, 1H).

Step 2: To a colorless mixture of 1,4-dioxaspiro[4.5]decane-8-carbaldehyde (1.00 eq, 4000 mg, 23.5 mmol) in DCM (80 mL) was added BAST (1.10 eq, 5 mL, 25.9 mmol) at 0° C., then the mixture was stirred at 15° C. for 12 h to give a yellow mixture. The mixture was dropwise added to saturated aq NaHCO₃ (50 mL), then the combined solution was extracted by DCM (3×60 mL), the combined organic layers were washed with brine, dried over anhydrous sodium sulfate, filtered, and concentrated in vacuo. The solution was purified by column on silica (10 g SiO2 cartridge, PE/EA=10%, detection at phosphomolybdic acid) and concentrated under reduced pressure to give colorless oil. 8-(difluoromethyl)-1,4-dioxaspiro[4.5]decane (3800 mg, 18.8 mmol, 79.92% yield) was obtained as colorless oil. ¹H NMR (400 MHz, CDCl3) δ=5.80-5.39 (m, 1H), 4.02-3.89 (m, 4H), 1.90-1.70 (m, 5H), 1.62-1.42 (m, 4H).

Step 3: To a colorless mixture of 8-(difluoromethyl)-1,4-dioxaspiro[4.5]decane (1.00 eq, 3.80 g, 19.8 mmol) in THF (5 mL) was added 1 M HCl (1.00 eq, 5.0 mL, 19.8 mmol), then the mixture was stirred at 40° C. for 24 hr. TLC (PE:EA=5:1, phosphomolybdic acid) showed the starting material was remained (Rf=0.5) and new spot was formed (Rf=0.4). The pH was adjusted to 7 with saturated aq NaHCO₃ (15 mL), the combined mixture was extracted by ethyl acetate (3×60 mL), the organic phase was concentrated to give a residue. The solution was purified by column on silica (8 g SiO2 cartridge, PE/EA=6%-10%, detection at phosphomolybdic acid) and concentrated under reduced pressure to give a colorless oil. 4-(difluoromethyl)cyclohexanone (1.50 g, 9.62 mmol, 48.65% yield) was obtained as colorless oil. ¹H NMR (400 MHz, CDCl3) δ=5.92-5.52 (m, 1H), 2.55-2.45 (m, 2H), 2.43-2.31 (m, 2H), 2.29-2.13 (m, 3H), 1.76-1.61 (m, 2H).

Step 4: To a colorless mixture of 4-(difluoromethyl)cyclohexanone (1.00 eq, 500 mg, 3.37 mmol) in THF (25 mL) was added LiHMDS (1.20 eq, 4.0 mL, 4.05 mmol) at −78° C. under N₂ atmosphere. then the mixture was stirred at −78° C. for 1 hr, 1,1,1-trifluoro-N-(2-pyridyl)-N-(trifluoromethylsulfonyl)methanesulfonamide (1.20 eq, 1451 mg, 4.05 mmol) was added at −78° C. under N₂ atmosphere, then the mixture was stirred at −78° C. for 0.5 hr, then the mixture was stirred at 15° C. for 12 hr to give red solution. TLC (PE:EA=5:1, phosphomolybdic acid) showed the starting material (Rf=0.3) was consumed completely and new spot (Rf=0.5) was found. The mixture was poured into NH₄Cl aqueous (30 mL) and extracted by ethyl acetate (3×50 mL), the combined organic layers were washed with brine, dried over anhydrous sodium sulfate, filtered, and concentrated in vacuo. The mixture was purified by prep-TLC (PE:EA=5:1, Rf=0.5), the purified solution was concentrated to give colorless oil. [4-(difluoromethyl)cyclohexen-1-yl] trifluoromethanesulfonate (589 mg, 2.06 mmol, 61.04% yield) was obtained as colorless oil. ¹H NMR (400 MHz, CDCl3) δ=5.85 (d, J=4.3 Hz, 1H), 5.82-5.76 (m, 1H), 5.71 (d, J=4.4 Hz, 1H), 5.57 (d, J=4.3 Hz, 1H), 2.55-2.31 (m, 3H), 2.27-1.99 (m, 3H), 1.76-1.60 (m, 1H).

Step 5: To a colorless mixture of [4-(difluoromethyl)cyclohexen-1-yl]trifluoromethanesulfonate (1.00 eq, 250 mg, 0.892 mmol) in 1,4-Dioxane (5 mL) was added BIS(PINACOLATO)DIBORON (1.50 eq, 340 mg, 1.34 mmol), potassium acetate (3.00 eq, 263 mg, 2.68 mmol), Pd(dppf)Cl₂ (0.100 eq, 65 mg, 0.0892 mmol), then the brown mixture was stirred at 90° C. for 12 hr under N₂ atmosphere to give black solution. TLC (PE:EA=8:1, cerium ammonium molybdate) showed the starting material was consumed completely and new spot was found (Rf=0.5). The reaction mixture was cooled to room temperature. The mixture was poured into water (20 mL) and extracted by ethyl acetate (3×30 mL), the combined organic layers were washed with brine, dried over anhydrous sodium sulfate, filtered, and concentrated in vacuo. The crude material was purified by column on silica (0.5 g SiO₂ cartridge, PE:EA=1% -10%, detection at cerium ammonium molybdate) and concentrated under reduced pressure to give white solid. 2-[4-(difluoromethyl)cyclohexen-1-yl]-4,4,5,5-tetramethyl-1,3,2-dioxaborolane (135 mg, 0.471 mmol, 52.76% yield) was obtained as white solid. ¹H NMR (400 MHz, CDCl3) δ=6.55 (br d, J=2.0 Hz, 1H), 5.77 (d, J=4.3 Hz, 1H), 5.63 (d, J=4.4 Hz, 1H), 5.49 (d, J=4.5 Hz, 1H), 2.36-1.94 (m, 6H), 1.92-1.81 (m, 1H), 1.27 (s, 12H).

Step 6: To a colorless mixture of 2-[4-(difluoromethyl)cyclohexen-1-yl]-4,4,5,5-tetramethyl-1,3,2-dioxaborolane (1.00 eq, 110 mg, 0.426 mmol) in 1,4-Dioxane (5 mL) and Water (1 mL) was added 2,4-dichloro-6,7-dimethyl-pyrido[2,3-d]pyrimidine (1.00 eq, 97 mg, 0.426 mmol), Cesium carbonate (3.00 eq, 417 mg, 1.28 mmol), Pd(dppf)Cl₂ DCM (0.0700 eq, 24 mg, 0.0298 mmol), then the reaction mixture was degassed with nitrogen for 3 times. The reaction mixture was stirred at 40° C. for 0.5 hr under N₂ atmosphere to give red solution. LCMS showed the starting material was consumed completely and 43% desired MS (323.9[M+1]⁺, ESI pos) was found. The reaction mixture was cooled to room temperature. The mixture was poured into water (30 mL) and extracted by ethyl acetate (3×30 mL), the combined organic layers were washed with brine, dried over anhydrous sodium sulfate, filtered, and concentrated in vacuo. The solution was purified by column on silica (0.2 g SiO₂ cartridge, PE:EA=42%, detection at 254 nm) and concentrated under reduced pressure to give red oil. 2-chloro-4-[4-(difluoromethyl)cyclohexen-1-yl]-6,7-dimethyl-pyrido[2,3-d]pyrimidine (112 mg, 0.311 mmol, 73.05% yield) was obtained as red oil. ¹H NMR (400 MHz, CDCl3) δ=8.12 (s, 1H), 6.21 (br s, 1H), 5.94 (d, J=4.2 Hz, 1H), 5.80 (d, J=4.2 Hz, 1H), 5.65 (d, J=4.4 Hz, 1H), 2.87-2.79 (m, 1H), 2.78 (s, 3H), 2.73-2.61 (m, 1H), 2.60-2.53 (m, 1H), 2.51 (s, 3H), 2.43-2.21 (m, 2H), 2.20-2.09 (m, 1H), 1.79-1.68 (m, 1H).

Step 7: To a yellow mixture of 2-chloro-4-[4-(difluoromethyl)cyclohexen-1-yl]-6,7-dimethyl-pyrido[2,3-d]pyrimidine (1.00 eq, 110 mg, 0.340 mmol) in DMSO (2 mL) was added (2S,6R)-2-(1-cyclopropylpyrazol-4-yl)-6-methyl-morpholine (1.50 eq, 106 mg, 0.510 mmol) and DIPEA (3.00 eq, 0.18 mL, 1.02 mmol), then the mixture was stirred at 100° C. for 1 hr to give yellow solution. LCMS showed the starting material was remained and 69.8% desired MS (495.2[M+1]⁺, ESI pos) was found. The reaction mixture was cooled to room temperature. The mixture was poured into water (30 mL) and extracted by ethyl acetate (3×30 mL), the combined organic layers were washed with brine, dried over anhydrous sodium sulfate, filtered, and concentrated in vacuo. The solution was purified by column on silica (0.5 g SiO₂ cartridge, PE:EA=1:1, detection at 254 nm) and concentrated under reduced pressure to give yellow solid. (2S,6R)-2-(1-cyclopropylpyrazol-4-yl)-4-[4-[4-(difluoromethyl)cyclohexen-1-yl]-6,7-dimethyl-pyrido[2,3-d]pyrimidin-2-yl]-6-methyl-morpholine (130 mg, 0.236 mmol, 69.4% yield) was obtained as yellow solid. LCMS showed 89% desired MS (495.3[M+1]⁺, ESI pos) was found. The solution was purified by prep-HPLC (NEU, column: Waters Xbridge 150*25 mm*5 um; mobile phase: water (NH₄HCO₃)-ACN; B %: 53%-83%, 8 min), the purified solution was lyophilized to give yellow solid. (2S,6R)-2-(1-cyclopropylpyrazol-4-yl)-4-[4-[4-(difluoromethyl)cyclohexen-1-yl]-6,7-dimethyl-pyrido[2,3-d]pyrimidin-2-yl]-6-methyl-morpholine;2,2,2-trifluoroacetic acid (2.8 mg, 0.00451 mmol, 8.92% yield) was obtained as yellow solid. LC-MS: Rt: 1.069 min; m/z: 495.1 [M+H]⁺. 100% purity at 214 nm. ¹H NMR (400 MHz, CDCl3) δ=7.99 (s, 1H), 7.55 (d, J=2.5 Hz, 2H), 6.14 (br s, 1H), 5.92 (d, J=3.8 Hz, 1H), 5.78 (d, J=4.1 Hz, 1H), 5.64 (d, J=4.3 Hz, 1H), 5.12-4.99 (m, 1H), 4.94 (br d, J=12.9 Hz, 1H), 4.57 (dd, J=2.4, 10.9 Hz, 1H), 3.85-3.73 (m, 1H), 3.58 (tt, J=3.8, 7.3 Hz, 1H), 3.02 (dd, J=11.1, 13.2 Hz, 1H), 2.83-2.69 (m, 5H), 2.61-2.45 (m, 2H), 2.39 (s, 3H), 2.35-2.19 (m, 2H), 2.12 (br d, J=11.9 Hz, 1H), 1.75-1.63 (m, 1H), 1.32 (d, J=6.1 Hz, 3H), 1.17-1.09 (m, 2H), 1.05-0.98 (m, 2H). HPLC: Rt: 2.735 min; 98.585% purity at 214 nm.

Step 8: To a yellow mixture of (2S,6R)-2-(1-cyclopropylpyrazol-4-yl)-4-[4-[4-(difluoromethyl)cyclohexen-1-yl]-6,7-dimethyl-pyrido[2,3-d]pyrimidin-2-yl]-6-methyl-morpholine (1.00 eq, 50 mg, 0.101 mmol) in Ethanol (1 mL) was added 10% Pd/C (1.00 eq, 5.0 mg, 0.101 mmol). Then the reaction mixture was degassed with H₂ for 3 times. The reaction mixture was stirred at 15° C. for 12 hr under H₂ atmosphere (15 psi). LCMS showed the starting material was consumed completely and 53.7% desired MS (501.3 [M+1]⁺, ESI pos) was found. The solution was filtered under N₂ atmosphere, the filtrate was concentrated under reduced pressure to give yellow oil. 2-[(2S,6R)-2-(1-cyclopropylpyrazol-4-yl)-6-methyl-morpholin-4-yl]-6-[4-(difluoromethyl)cyclohexyl]-5-ethyl-N-sec-butyl-pyrimidin-4-amine (50 mg, 0.0513 mmol, 50.73% yield) was obtained as yellow oil.

Step 9: To a colorless mixture of (2S,6R)-2-(1-cyclopropylpyrazol-4-yl)-4-[4-[4-(difluoromethyl)cyclohexyl]-6,7-dimethyl-5,6,7,8-tetrahydropyrido[2,3-d]pyrimidin-2-yl]-6-methyl-morpholine (1.00 eq, 40 mg, 0.0799 mmol) in MeCN (3 mL) was added NBS (2.00 eq, 28 mg, 0.160 mmol), Na₂CO₃ (3.00 eq, 25 mg, 0.240 mmol), then the mixture was stirred at 15° C. for 12 hr. LCMS showed the starting material was consumed completely and 59% desired MS (497.2[M+1]⁺, ESI pos) was found. The mixture was poured into Na₂SO₃ (20 mL) and extracted by ethyl acetate (3×30 mL), the combined organic layers were washed with brine, dried over anhydrous sodium sulfate, filtered, and concentrated in vacuo. The solution was purified by prep-TLC (EA), the purified solution was concentrated to give yellow solid. The solution was lyophilized to give yellow solid. (2S,6R)-2-(1-cyclopropylpyrazol-4-yl)-4-[4-[4-(difluoromethyl)cyclohexyl]-6,7-dimethyl-pyrido[2,3-d]pyrimidin-2-yl]-6-methyl-morpholine (8.1 mg, 0.0155 mmol, 19.40% yield) was obtained as yellow solid. LCMS: Rt: 0.843 min; m/z: 497.4 [M+H]⁺. 97.789% purity at 214 nm. ¹H NMR (400 MHz, CDCl3) δ=8.03 (br s, 1H), 7.59-7.50 (m, 2H), 6.21-5.31 (m, 1H), 5.22-4.82 (m, 2H), 4.56 (br d, J=10.5 Hz, 1H), 3.83-3.74 (m, 1H), 3.64-3.23 (m, 2H), 3.10-3.01 (m, 1H), 2.88-2.80 (m, 3H), 2.46-2.37 (m, 3H), 2.10-1.92 (m, 5H), 1.91-1.70 (m, 3H), 1.52-1.42 (m, 1H), 1.33 (d, J=6.2 Hz, 3H), 1.26 (br d, J=4.5 Hz, 1H), 1.14 (br d, J=1.1 Hz, 2H), 1.02 (br d, J=6.8 Hz, 2H) HPLC: Rt: 2.761 min; 91.781% purity at 214 nm.

### Synthesis of Compound I-1313

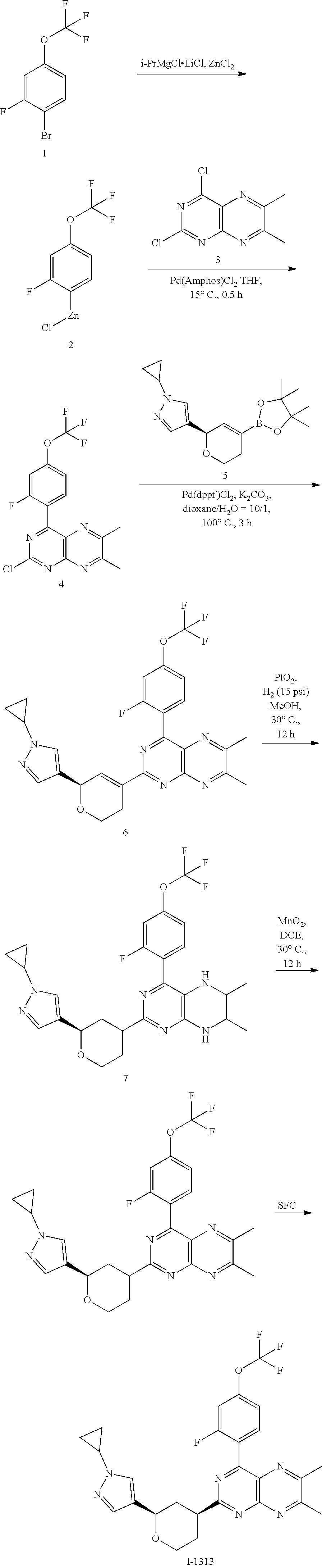

Step 1: To a solution of 1-bromo-2-fluoro-4-(trifluoromethoxy)benzene (1.00 eq, 2000 mg, 7.72 mmol) in THF (20 mL) was added iPrMgCl LiCl (1.11 eq, 6.6 mL, 8.54 mmol) at 0° C. under N₂. The mixture was stirred at 15° C. for 1.5 h. ZnCl₂ (0.5 M in THF, 1.21 eq, 19 mL, 9.38 mmol) was added at −78° C. under N₂ and the mixture was stirred at 15° C. for 1 h. The reaction mixture was used directly for the next step.

Step 2: A sealed bottle under N₂ atmosphere was charged with 2,4-dichloro-6,7-dimethyl-pteridine (1.00 eq, 500 mg, 2.18 mmol) and Pd(Amphos)Cl₂ (0.0500 eq, 77 mg, 0.109 mmol) and THF (20 mL) and purged with N₂ three times, then cooled to 0° C., chloro-[2-fluoro-4-(trifluoromethoxy)phenyl]zinc (1.50 eq, 19 mL, 3.27 mmol) was added dropwise to the reaction solution at 0° C., then warmed to 20° C. and stirred for 0.5 hr. The reaction solution was changed from orange to dark purple. LCMS showed that 10% the starting material remained and 69% of the desired mass was detected. (373.1, [M+H]⁺, ESI pos). The reaction solution was combined and quenched by saturated NH₄Cl solution (100 mL), then extracted with EtOAc (100 mL×2) and the organics was evaporated under reduced pressure to give the residue, which was then purified with Flash column (PE:EA=0-40%, PE/EtOAc=3/1, Rf=0.5) and concentrated in vacuo to give 2-chloro-4-(2-fluoro-4-(trifluoromethoxy)phenyl)-6,7-dimethylpteridine (770 mg, 2.07 mmol, 94.65% yield) as yellow solid. (M+H)⁺=373.0; purity=85% (220 nm). Retention time=0.872 min. ¹H NMR (400 MHz, CDCl₃) δ=7.83 (t, J=8.1 Hz, 1H), 7.24 (br d, J=8.4 Hz, 1H), 7.18-7.13 (m, 1H), 2.90-2.87 (m, 3H), 2.79-2.76 (m, 3H).

Step 3: To a solution of 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 653 mg, 2.07 mmol), 2-chloro-4-[2-fluoro-4-(trifluoromethoxy)phenyl]-6,7-dimethyl-pteridine (1.00 eq, 700 mg, 1.88 mmol) and K₂CO₃ (3.00 eq, 473 mg, 5.63 mmol) in 1,4-Dioxane (40 mL) and Water (4 mL) was added Pd(dppf)Cl₂ (0.0909 eq, 125 mg, 0.171 mmol) at 20° C. The mixture was stirred at 100° C. for 3 h. LCMS showed the starting material was consumed completely and a major peak with desired MS (72.3%, MS: 527.2 [M+H]⁺, ESI pos).The reaction mixture was partitioned between EtOAc (100 mL) and water (100 mL).The combined organic layers were dried over Na₂SO₄, filtered and concentrated under reduced pressure to give a residue. The crude product was purified by column chromatography on silica gel chromatography (PE/EtOAc=0-100%, PE/EtOAc=1/1, the desired product R_{f}=0.6) to afford (R)-2-(6-(1-cyclopropyl-1H-pyrazol-4-yl)-3,6-dihydro-2H-pyran-4-yl)-4-(2-fluoro-4-(trifluoromethoxy)phenyl)-6,7-dimethylpteridine (760 mg, 1.44 mmol, 76.86% yield) as brown solid. (M+H)⁺=527.0; purity=96% (220 nm). Retention time=0.875 min. ¹H NMR (400 MHz, CDCl₃) δ=7.83 (t, J=8.1 Hz, 1H), 7.66 (d, J=2.4 Hz, 1H), 7.52 (d, J=11.6 Hz, 2H), 7.26-7.11 (m, 2H), 5.46 (d, J=2.6 Hz, 1H), 4.16-4.11 (m, 1H), 3.94 (ddd, J=4.9, 7.0, 11.6 Hz, 1H), 3.56 (tt, J=3.7, 7.3 Hz, 1H), 3.04-2.93 (m, 2H), 2.85 (s, 3H), 2.74 (s, 3H), 1.13-1.09 (m, 2H), 1.02-0.96 (m, 2H).

Step 4: To a solution of 2-[(6R)-6-(1-cyclopropylpyrazol-4-yl)-3,6-dihydro-2H-pyran-4-yl]-4-[2-fluoro-4-(trifluoromethoxy)phenyl]-6,7-dimethyl-pteridine (1.00 eq, 100 mg, 0.190 mmol) in Methanol (5 mL) was added PtO₂ (0.517 eq, 22 mg, 0.0982 mmol) under N₂ atmosphere. The mixture was purged with H₂ (15 psi) 3 times, then the mixture was stirred at 30° C. for 12 h. LCMS (5-95AB/1.5 min): RT=0.837 min, 533.2=[M+H]⁺, ESI⁺ showed 98.8% of desired product. The reaction mixture was filtered and concentrated under reduced pressure to give crude 2-((2R)-2-(1-cyclopropyl-1H-pyrazol-4-yl)tetrahydro-2H-pyran-4-yl)-4-(2-fluoro-4-(trifluoromethoxy)phenyl)-6,7-dimethyl-5,6,7,8-tetrahydropteridine (101 mg, 0.190 mmol, 100.00% yield). (M+H)⁺=533.2; purity=98.8% (220 nm). Retention time=0.837 min.

Step 5: To a solution of 2-[(2R)-2-(1-cyclopropylpyrazol-4-yl)tetrahydropyran-4-yl]-4-[2-fluoro-4-(trifluoromethoxy)phenyl]-6,7-dimethyl-5,6,7,8-tetrahydropteridine (1.00 eq, 100 mg, 0.188 mmol) in DCE (8 mL) was added MnO₂ (20.0 eq, 327 mg, 3.76 mmol), then the mixture was stirred at 30° C. for 12 h. LCMS showed the starting material still remained and a peak with desired MS (9%, MS: 529.0 [M+H]⁺, ESI pos). The reaction mixture was filtered and concentrated under reduced pressure to give a residue. A mixture of the residue in DCE (8 mL) was added MnO₂ (20.0 eq, 327 mg, 3.76 mmol) then the mixture was stirred at 30° C. for 12 h. LCMS showed some starting material remained and a peak with desired MS (21%, MS: 529.0 [M+H]⁺,ESI pos). The reaction mixture was filtered and concentrated under reduced pressure to give a residue. A mixture of the residue in DCE (8 mL) was added MnO₂ (20.0 eq, 327 mg, 3.76 mmol), then the mixture was stirred at 30° C. for 12 h. LCMS showed the starting material was consumed with a major peak with desired MS (96%, MS: 529.1 [M+H]⁺, ESI pos). The reaction mixture was filtered and the filtrate was concentrated under reduced pressure to give a residue. The residue was purified by prep-HPLC (Phenomenex C18 75×30 mm×3 um; mobile phase: [water (0.1% FA)-ACN]; B %: 42%-72%, 9 min) and lyophilized to afford 65.9 mg. 2-((2R)-2-(1-cyclopropyl-1H-pyrazol-4-yl)tetrahydro-2H-pyran-4-yl)-4-(2-fluoro-4-(trifluoromethoxy)phenyl)-6,7-dimethylpteridine (5.9 mg, 0.0112 mmol, 5.94% yield) was delivered, checked by LCMS (M+H)⁺=529.1; purity=100% (220 nm). Retention time=0.973 min. ¹H NMR (400 MHz, CDCl₃) δ=7.80 (t, J=8.1 Hz, 1H), 7.51 (s, 2H), 7.24 (br d, J=8.7 Hz, 1H), 7.16 (br d, J=10.3 Hz, 1H), 4.56 (br d, J=9.9 Hz, 1H), 4.32-4.23 (m, 1H), 3.87-3.77 (m, 1H), 3.61-3.50 (m, 2H), 2.86 (s, 3H), 2.75 (s, 3H), 2.45 (br d, J=13.6 Hz, 1H), 2.27-2.15 (m, 3H), 1.16-1.05 (m, 2H), 1.03-0.96 (m, 2H).

Step 6: The 2-[(2R)-2-(1-cyclopropylpyrazol-4-yl)tetrahydropyran-4-yl]-4-[2-fluoro-4-(trifluoromethoxy)phenyl]-6,7-dimethyl-pteridine (1.00 eq, 60 mg, 0.114 mmol) was purified by SFC [Column: Chiralcel OD-3 50×4.6 mm I.D., 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35C; Back Pressure: 100 Bar] to afford 2-((2R,4S)-2-(1-cyclopropyl-1H-pyrazol-4-yl)tetrahydro-2H-pyran-4-yl)-4-(2-fluoro-4-(trifluoromethoxy)phenyl)-6,7-dimethylpteridine (28 mg, 0.0525 mmol, 46.20% yield) as off-white solid, checked by LCMS (M+H)⁺=529.3; purity=99% (220 nm). Retention time=0.979 min. ¹H NMR (400 MHz, CDCl₃) δ=7.80 (t, J=8.1 Hz, 1H), 7.52 (s, 2H), 7.24 (br d, J=8.8 Hz, 1H), 7.16 (br d, J=9.9 Hz, 1H), 4.56 (dd, J=1.8, 11.4 Hz, 1H), 4.27 (td, J=3.1, 11.2 Hz, 1H), 3.86-3.78 (m, 1H), 3.60-3.51 (m, 2H), 2.86 (s, 3H), 2.75 (s, 3H), 2.45 (br d, J=13.4 Hz, 1H), 2.24-2.17 (m, 3H), 1.13-1.08 (m, 2H), 1.03-0.98 (m, 2H).

### Synthesis of Compound I-1318

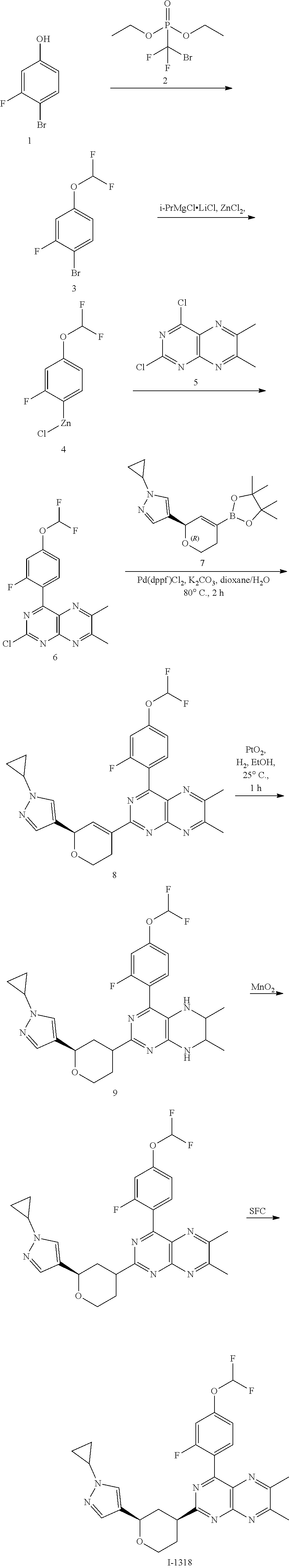

Step 1: To a mixture of 4-bromo-3-fluoro-phenol (1.00 eq, 5000 mg, 26.2 mmol) in MeCN (250 mL) was added KOH (10.0 eq, 14688 mg, 262 mmol) in H₂O (60 mL) was added, followed by 1-[[bromo(difluoro)methyl]-ethoxy-phosphoryl]oxyethane (4.00 eq, 27959 mg, 105 mmol). The mixture was stirred at 30° C. for 12 h. LCMS showed the starting material was consumed completely and one major peak was detected (no mass signal). The mixture was diluted with 100 mL H₂O, extracted with EA (100 mL*3), the combine organic layers was dried over anhydrous Na₂SO₄, filtered, and concentrated under reduced pressure to give the residue. The residue was purified by silica column chromatography (PE:EA=1:0 to 3:1, TLC (DP): PE:EA=3:1, R_{f}=0.5) to give the 1-bromo-4-(difluoromethoxy)-2-fluoro-benzene (4200 mg, 17.4 mmol, 66.57% yield) as colorless oil. 1H NMR (400 MHz, CDCl3) δ=7.55 (dd, J=7.8, 8.7 Hz, 1H), 6.97 (dd, J=2.70, 9.0 Hz, 1H), 6.90-6.82 (m, 1H), 6.73-6.31 (m, 1H).

Step 2: To a solution of 1-bromo-4-(difluoromethoxy)-2-fluoro-benzene (1.00 eq, 2000 mg, 8.30 mmol) in THF (20 mL) was added iPrMgCl LiCl (1.11 eq, 7.1 mL, 9.18 mmol) at 0° C. under N₂. The mixture was stirred at 15° C. for 1.5 h. ZnCl₂ (0.5 M in THF, 1.21 eq, 20 mL, 10.1 mmol) was added at −78° C. under N₂ and the mixture was stirred at 15° C. for 1 h. the reaction mixture was used directly for the next step.

Step 3: A sealed bottle under N₂ atmosphere was charged with 2,4-dichloro-6,7-dimethyl-pteridine (1.00 eq, 500 mg, 2.18 mmol) and PdCl₂(Amphos) (0.0500 eq, 77 mg, 0.109 mmol) and THF (5 mL) and purged with N₂ three times, then cooled to 0° C., chloro-[4-(difluoromethoxy)-2-fluoro-phenyl]zinc (1.30 eq, 16 mL, 2.84 mmol) was added dropwise to the reaction solution at 0° C., then warmed to 25° C. and stirred for 1 h. The reaction solution was changed from orange to dark purple, LCMS showed 75% of desired product was detected (MS: 355.1 [M+H]⁺, ESI pos, RT=0.911 min). The reaction solution was quenched by saturation NH₄Cl solution (100 mL), extracted with EtOAc (150 mL * 3), the combine organic layers was dried over anhydrous Na₂SO₄, filtered, and concentrated under reduced pressure to give the residue. The residue was purified by Flash column (PE:EA=1:0 to 1:1, TLC (DP): PE:EA=1:1, R_{f}=0.5) and concentrated under reduced pressure to give 2-chloro-4-[4-(difluoromethoxy)-2-fluoro-phenyl]-6,7-dimethyl-pteridine (600 mg, 1.69 mmol, 77.50% yield) as yellow solid, checked by LCMS (355.1 [M+H]⁺, ESI pos, RT=0.897 min). LCMS: 355.1 [M+H]⁺, ESI pos, RT=0.897 min. ¹H NMR (400 MHz, CDCl3) δ=7.81 (t, J=8.10 Hz, 1H), 7.19-7.10 (m, 1H), 7.09-7.02 (m, 1H), 6.85-6.44 (m, 1H), 2.86 (s, 3H), 2.76 (s, 3H).

Step 4: To a solution of 1-cyclopropyl-4-[(6R)-4-(4,4,5,5-tetramethyl-1,3,2-dioxaborolan-2-yl)-3,6-dihydro-2H-pyran-6-yl]pyrazole (1.15 eq, 595 mg, 1.88 mmol), 2-chloro-4-[4-(difluoromethoxy)-2-fluoro-phenyl]-6,7-dimethyl-pteridine (1.00 eq, 580 mg, 1.64 mmol) and K₂CO₃ (3.00 eq, 412 mg, 4.91 mmol) in 1,4-Dioxane (30 mL) and Water (3 mL) was added Pd(dppf)Cl₂ DCM (0.120 eq, 144 mg, 0.196 mmol). The reaction mixture was stirred at 80° C. for 12 h under N₂ atmosphere. LCMS showed the starting material was consumed completely and 85% of desired product was detected (509.2, [M+H]⁺, ESI⁺, RT=0.955 min). The reaction mixture was poured into 250 mL H₂O, extracted with EA (150 mL×3), the combine organic layers was dried over anhydrous Na₂SO₄, filtered, and concentrated under reduced pressure to give the residue. The residue was purified by silica column chromatography (PE:EA=1:0 to 0:1, TLC (DP): PE:EA=1:1, R_{f}=0.3) to give the 4-[4-(difluoromethoxy)-2-fluoro-phenyl]-6,7-dimethyl-2-[(6R)-6-(1-cyclopropylpyrazol-4-yl)-3,6-dihydro-2H-pyran-4-yl]pteridine (600 mg, 1.18 mmol, 72.16% yield) as red solid, LCMS: (509.2, [M+H]⁺, ESI⁺, RT=0.950 min). LCMS: 509.2, [M+H]⁺, ESI⁺, RT=0.950 min. 1H NMR (400 MHz, CDCl3) δ=7.80 (t, J=8.1 Hz, 1H), 7.66 (d, J=2.3 Hz, 1H), 7.52 (d, J=12.6 Hz, 2H), 7.12 (dd, J=2.1, 8.5 Hz, 1H), 7.05 (dd, J=2.2, 10.3 Hz, 1H), 6.83-6.44 (m, 1H), 5.46 (d, J=2.6 Hz, 1H), 4.16-4.12 (m, 1H), 3.94 (ddd, J=4.9, 6.9, 11.6 Hz, 1H), 3.57 (tt, J=3.7, 7.3 Hz, 1H), 3.02-2.94 (m, 2H), 2.85 (s, 3H), 2.74 (s, 3H), 1.15-1.09 (m, 2H), 1.03-0.96 (m, 2H)).

Step 5: To a solution of 2-[(6R)-6-(1-cyclopropylpyrazol-4-yl)-3,6-dihydro-2H-pyran-4-yl]-4-[4-(difluoromethoxy)-2-fluoro-phenyl]-6,7-dimethyl-pteridine (1.00 eq, 500 mg, 0.983 mmol) in Ethanol (20 mL) was added PtO₂ (0.515 eq, 115 mg, 0.506 mmol) under N₂ atmosphere. The mixture was purged with H₂ (15 psi) 3 times, then the mixture was stirred at 30° C. under H₂ (15 psi) for 12 h. LCMS showed the starting material was consumed completely and one major peak (desired product) was detected (515.2, [M+H]⁺, ESI⁺, RT=0.950 min). The mixture was filtered and the filter cake was washed with EtOH (20 mL×3), the combine organic layers was concentrated under reduced pressure to give the 2-[(2R)-2-(1-cyclopropylpyrazol-4-yl)tetrahydropyran-4-yl]-4-[4-(difluoromethoxy)-2-fluoro-phenyl]-6,7-dimethyl-5,6,7,8-tetrahydropteridine (500 mg, 0.972 mmol, 98.83% yield) as yellow solid and the product was used next step directly. LCMS: 515.2, [M+H]⁺, ESI⁺, RT=0.950 min.

Step 6: To a solution of 2-[(2R)-2-(1-cyclopropylpyrazol-4-yl)tetrahydropyran-4-yl]-4-[4-(difluoromethoxy)-2-fluoro-phenyl]-6,7-dimethyl-5,6,7,8-tetrahydropteridine (1.00 eq, 500 mg, 0.972 mmol) in DCE (80 mL) was added MnO₂ (20.0 eq, 1690 mg, 19.4 mmol). The mixture was stirred at 30° C. for 12 h. LCMS showed 50% of intermediate product (513.2, [M+H]⁺, ESI⁺, RT=0.805 min) and 37% of desired product (511.2, [M+H]⁺, ESI⁺, RT=0.935 min) was detected. The mixture was filtered and then the organic layers was concentrated under reduced pressure to give the residue. The residue was redissolved in DCE (80 mL) was added MnO₂ (20.0 eq, 1690 mg, 19.4 mmol), then the mixture was stirred at 30° C. for 12 h. LCMS showed 22% of intermediate product (513.2, [M+H]⁺, ESI⁺, RT=0.805 min) and 64% of desired product (511.2, [M+H]⁺, ESI⁺, RT=0.935 min) was detected. The mixture was stirred at 30° C. for another 12 h. LCMS showed 95% of desired product (511.2, [M+H]⁺, ESI⁺, RT=0.935 min) was detected. The mixture was filtered and concentrated under reduced pressure to give the residue. The residue was purified by Prep-HPLC (Phenomenex luna C18 150*40 mm*15 um, water (FA)-ACN) and lyophilized to give the 2-[(2R,4S)-2-(1-cyclopropylpyrazol-4-yl)tetrahydropyran-4-yl]-4-[4-(difluoromethoxy)-2-fluoro-phenyl]-6,7-dimethyl-pteridine (290 mg, 0.568 mmol, 58.46% yield) as yellow solid, LCMS (511.2, [M+H]⁺, ESI⁺, RT=0.938 min). SFC showed 90.5% ee. LCMS: 511.2, [M+H]⁺, ESI⁺, RT=0.938 min. 1H NMR (400 MHz, CDCl3) δ=7.78 (t, J=8.1 Hz, 1H), 7.51 (s, 2H), 7.13 (dd, J=2.0, 8.5 Hz, 1H), 7.06 (dd, J=2.2, 10.3 Hz, 1H), 6.84-6.41 (m, 1H), 4.56 (dd, J=1.9, 11.4 Hz, 1H), 4.27 (td, J=3.1, 11.2 Hz, 1H), 3.88-3.77 (m, 1H), 3.61-3.49 (m, 2H), 2.89-2.82 (m, 3H), 2.74 (s, 3H), 2.45 (br d, J=13.4 Hz, 1H), 2.28-2.22 (m, 1H), 2.21-2.13 (m, 2H), 1.15-0.91 (m, 4H).

Step 7: The product (80 mg) was purified by SFC (Column: Chiralpak AD-3 50×4.6 mm I.D., 3 um Mobile phase: Phase A for CO₂, and Phase B for EtOH (0.05% DEA); Gradient elution:EtOH (0.05% DEA) in CO₂ from 5% to 40% Flow rate: 3 mL/min; Detector: PDA Column Temp: 35C; Back Pressure: 100 Bar”) and lyophilized to give the 2-[(2R,4S)-2-(1-cyclopropylpyrazol-4-yl)tetrahydropyran-4-yl]-4-[4-(difluoromethoxy)-2-fluoro-phenyl]-6,7-dimethyl-pteridine (47 mg, 0.0910 mmol, 58.08% yield) as white solid, LCMS: (511.2, [M+H]⁺, ESI⁺, RT=0.936 min). SFC showed 100% ee. LCMS: 511.2, [M+H]⁺, ESI⁺, RT=0.936 min. 1H NMR (400 MHz, CDCl3) δ=7.78 (t, J=8.1 Hz, 1H), 7.51 (d, J=1.4 Hz, 2H), 7.13 (dd, J=1.9, 8.4 Hz, 1H), 7.08-7.03 (m, 1H), 6.85-6.42 (m, 1H), 4.56 (dd, J=1.9, 11.4 Hz, 1H), 4.27 (td, J=3.0, 11.1 Hz, 1H), 3.88-3.73 (m, 1H), 3.61-3.49 (m, 2H), 2.45 (br d, J=13.3 Hz, 1H), 2.27-2.17 (m, 3H), 1.14-1.07 (m, 2H), 1.03-0.95 (m, 2H).

### Synthesis of Compound I-1323

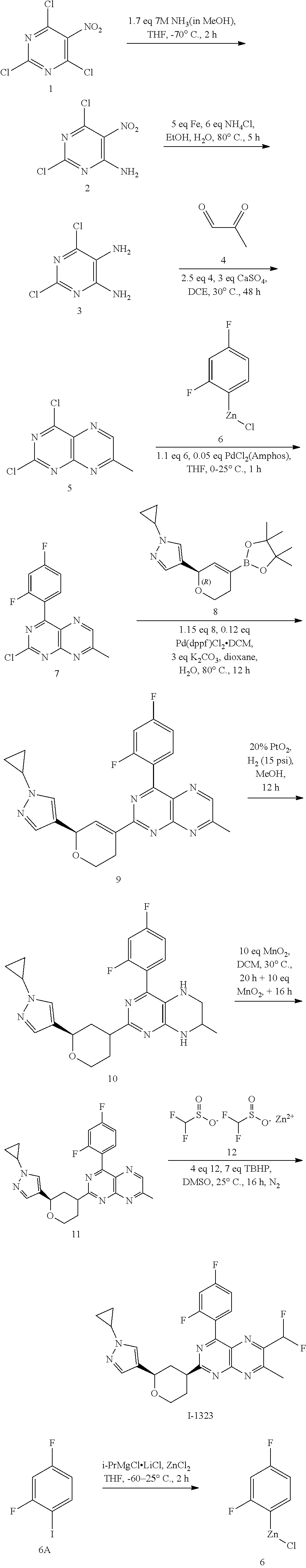

Step 1: To a solution of 2,4,6-trichloro-5-nitro-pyrimidine (1.00 eq, 13.00 g, 56.9 mmol) in THF (200 mL) was added a solution of NH₃ in MeOH (1.70 eq, 14 mL, 96.8 mmol) dropwise at −65° C. The reaction was stirred at −65° C. for 3 h. LCMS (5-95AB/1.5 min): RT=0.510 min, 209.0=[M+H]⁺, ESI⁺ showed 74.3% of desired product. The reaction mixture was adjusted to pH=5-6 with glacial acetic acid. The reaction mixture was concentrated under reduced pressure to afford a crude product 2, 6-dichloro-5-nitro-pyrimidin-4-amine (15.30 g, 54.2 mmol, 95.19% yield) as yellow solid. The crude product was used to next step without further purification.

Step 2: To a solution of 2,6-dichloro-5-nitro-pyrimidin-4-amine (1.00 eq, 15.30 g, 54.2 mmol) in Ethanol (150 mL) and Water (30 mL) was added Fe (5.00 eq, 15128 mg, 271 mmol) and NH₄Cl (6.00 eq, 17387 mg, 325 mmol). The reaction mixture was stirred at 60° C. for 12 hours. TLC (PE:EtOAc=0:1) showed the starting material was consumed and the desired product was formed (R_{f}=0.45). The reaction was diluted with MeOH (500 mL) and stirred for 30 min. The suspension was filtered through a pad of celite. The filter cake was washed with MeOH (200 mL). The filtrate was concentrated under reduced pressure to afford a residue. The residue was diluted with water (100 mL) and then extracted with ethyl acetate (100 mL×3). The combined organic layers were washed with brine, dried over Na₂SO₄, filtered, and concentrated under reduced pressure to give a residue. The residue was purified by flash chromatography on silica gel eluting with PE/EtOAc (1:1 to 1:3) (TLC, PE:EtOAc=0:1, R_{f}=0.45) to afford 2,6-dichloropyrimidine-4,5-diamine (6.40 g, 33.1 mmol, 61.11% yield) as yellow solid. LCMS: Rt: 0.250 min; [M+H]⁺=179.0; 92.6% purity at 220 nm.

Step 3: To a solution of 2-oxopropanal (2.50 eq, 1006 mg, 14.0 mmol) in DCE (60 mL) was added CaSO₄ (3.00 eq, 2281 mg, 16.8 mmol) followed by 2,6-dichloropyrimidine-4,5-diamine (1.00 eq, 1000 mg, 5.59 mmol). The reaction mixture was stirred at 25° C. for 48 hr. LCMS (5-60AB/1.5 min): RT=0.786 min, 215.0=[M+H]⁺, ESI⁺ showed 97.4% of desired product MS. The reaction was diluted with EtOAc (100 mL) and then filtered through a pad of celite. The filter cake was washed with MeOH (100 mL) and EtOAc (100 mL) and DCM (100 mL). The combine filtrate was concentrated under reduced pressure to afford a residue. The residue was purified by flash chromatography on silica gel eluting with PE/EtOAc (3:1) (TLC, PE:EtOAc=3:1, R_{f}=0.40) to afford 2,4-dichloro-7-methyl-pteridine (1000 mg, 4.65 mmol, 83.24% yield) as yellow solid. LCMS: Rt: 0.802 min; [M+H]⁺=215.0; 99.1% purity at 220 nm.

Step 4: A three necked was equipped with 2,4-difluoro-1-iodo-benzene (1.00 eq, 6000 mg, 25.0 mmol), the flash was sealed and purged with N₂ for 3 times, THF (60 mL) was added and the solution was cooled to −40° C. with stirring. iPrMgCl·LiCl (1.3 M in THF) (1.10 eq, 21 mL, 27.5 mmol) was added dropwise at −40° C. and the mixture was stirred for 30 min at this temperature, The reaction mixture was further cooled to −60° C. and ZnCl₂ (0.5 M in THF) (1.00 eq, 50 mL, 25.0 mmol) was added dropwise, the reaction solution turned into white floc, the reaction mixture was allowed to warm to room temperature gradually and stirred for 1 h. The mixture was used directly without further workup and purification.

Step 5: A sealed bottle under N₂ atmosphere was charged with 2,4-dichloro-7-methyl-pteridine (1.00 eq, 1000 mg, 4.65 mmol) and PdCl₂(Amphos) (0.0600 eq, 198 mg, 0.279 mmol) and THF (10 mL) and purged with N₂ three times, then cooled to 0° C., chloro-(2,4-difluorophenyl)zinc (1.10 eq, 19 mL, 5.12 mmol) was added dropwise to the reaction solution at 0° C., then warmed to 25° C. and stirred for 2 hrs. LCMS (5-95AB/1.5 min): RT=0.846 min, 293.0=[M+H]⁺, ESI⁺ showed 61.8% of desired product. The reaction was diluted with water (50 mL) and then extracted with ethyl acetate (50 mL×3). The combined organic layers were washed with brine, dried over Na₂SO₄, filtered, and concentrated under reduced pressure to give a residue. The residue was purified by flash chromatography on silica gel eluting with PE/EtOAc (3:1) (TLC, PE:EtOAc=3:1, R_{f}=0.45) to afford 2-chloro-4-(2,4-difluorophenyl)-7-methyl-pteridine (915 mg, 2.59 mmol, 55.80% yield) as brown solid. LCMS: Rt: 0.849 min; [M+H]⁺=293.0; 83.2% purity at 220 nm.

Step 6: To a solution of 2-chloro-4-(2,4-difluorophenyl)-7-methyl-pteridine (1.00 eq, 860 mg, 2.35 mmol), Pd(dppf)Cl₂ DCM (0.120 eq, 206 mg, 0.282 mmol) and K₂CO₃ (3.00 eq, 975 mg, 7.05 mmol) in 1,4-Dioxane (30 mL) and Water (3 mL) was added Pd(dppf)Cl₂ DCM (0.120 eq, 206 mg, 0.282 mmol). The reaction mixture was stirred at 80° C. for 5 hours under N₂ atmosphere. LCMS (5-95AB/1.5 min): RT=0.652 min, 447.0=[M+H]⁺, ESI⁺ showed 28.9% of desired product. The reaction mixture was combined with for further purification. The reaction was diluted with water (100 mL) and then extracted with ethyl acetate (100 mL×3). The combined organic layers were washed with brine, dried over Na₂SO₄, filtered, and concentrated under reduced pressure to give a residue. The residue was purified by flash chromatography on silica gel eluting with PE/EtOAc (1:3) (TLC, PE:EtOAc=0:1, R_{f}=0.50) to afford 4-(2,4-difluorophenyl)-7-methyl-2-[rac-(6R)-6-(1-cyclopropylpyrazol-4-yl)-3,6-dihydro-2H-pyran-4-yl]pteridine (500 mg, 0.999 mmol, 42.50% yield) as a purple solid. LCMS: Rt: 0.648 min; [M+H]⁺=447.0; 89.2% purity at 220 nm.

Step 7: To a solution of 2-[(6R)-6-(1-cyclopropylpyrazol-4-yl)-3,6-dihydro-2H-pyran-4-yl]-4-(2,4-difluorophenyl)-7-methyl-pteridine (1.00 eq, 500 mg, 1.12 mmol) in Methanol (20 mL) was added PtO₂ (0.492 eq, 125 mg, 0.551 mmol) under N₂ atmosphere. The mixture was purged with H₂ (15 psi) 3 times, then the mixture was stirred at 30° C. under H₂ (15 psi) for 48 hours. LCMS (5-95AB/1.5 min): RT=0.760 min, 453.2=[M+H]⁺, ESI⁺ showed 98% of desired product and HPLC (10-80AB/1.5 min): RT=1.433 min, showed 86.8% of desired product. The reaction mixture was filtered through a pad of celite. The filter cake was washed with MeOH (100 mL) and EtOAc (100 mL). The filtrate was concentrated under reduced pressure to afford crude product 2-[(2R)-2-(1-cyclopropylpyrazol-4-yl)tetrahydropyran-4-yl]-4-(2,4-difluorophenyl)-7-methyl-5,6,7,8-tetrahydropteridine (520 mg, 0.997 mmol, 89.07% yield) as yellow gum. The crude product was used to next step without further purification.

Step 8: To a solution of 2-[(2R)-2-(1-cyclopropylpyrazol-4-yl)tetrahydropyran-4-yl]-4-(2,4-difluorophenyl)-7-methyl-5,6,7,8-tetrahydropteridine (1.00 eq, 400 mg, 0.767 mmol) in DCE (50 mL) was added MnO₂ (25.0 eq, 1668 mg, 19.2 mmol) at 30° C. and stirred for 16 hours. LCMS (5-95AB/1.5 min): RT=0.890 min, 449.2=[M+H]⁺, ESI⁺ showed 12.8% of desired product and RT=0.766 min, 453.2=[M+H]⁺, ESI⁺ showed 19.7% of starting material. The reaction mixture was filtered through a pad of celite. The filter cake was washed with DCM (80 mL). The filtrate was concentrated under reduced pressure to afford a residue. The residue was dissolved in DCE (40 mL) and MnO₂ (25.0 eq, 1668 mg, 19.2 mmol) was added. The reaction mixture was stirred at 30° C. for 16 hours. LCMS (5-95AB/1.5 min): RT=0.891 min, 449.2=[M+H]⁺, ESI⁺ showed 46.2% of desired product. The reaction mixture was filtered through a pad of celite. The filter cake was washed with DCM (80 mL). The filtrate was concentrated under reduced pressure to afford a residue. The residue was purified by flash chromatography on silica gel eluting with [petroleum ether]/[ethyl acetate] (1:2 to 1:3) to afford 2-[(2R)-2-(1-cyclopropylpyrazol-4-yl)tetrahydropyran-4-yl]-4-(2,4-difluorophenyl)-7-methyl-pteridine (155 mg, 0.290 mmol, 37.84% yield) as yellow solid. LCMS: Rt: 0.889 min; [M+H]⁺=449.2; 83.4% purity at 220 nm. ¹H NMR (400 MHz, CDCl3) δ ppm 0.96-1.02 (m, 2H) 1.08-1.12 (m, 2H) 2.18-2.25 (m, 3H) 2.46 (brd, J=13.51 Hz, 1H)_{2.91} (s, 3H) 3.53-3.61 (m, 2H) 3.78-3.87 (m, 1H)_{4.28} (dt, J=11.16, 3.17 Hz, 1H) 4.52-4.62 (m, 1H) 6.98-7.06 (m, 1H) 7.11 (td, J=8.25, 1.88 Hz, 1H) 7.51 (d, J=1.50 Hz, 2H) 7.72-7.80 (m, 1H) 8.75-8.92 (m, 1H).

Step 9: To a solution of 2-[(2R)-2-(1-cyclopropylpyrazol-4-yl)tetrahydropyran-4-yl]-4-(2,4-difluorophenyl)-7-methyl-pteridine (1.00 eq, 100 mg, 0.223 mmol) and zinc difluoromethanesulfinate (4.00 eq, 262 mg, 0.892 mmol) in DMSO (3 mL) at 25° C. was added tert-butylhydroperoxide (7.00 eq, 201 mg, 1.56 mmol) with vigorous stirring and bubbled with N₂ for 30 seconds. The reaction solution was stirred at 25° C. for 12 hrs. LCMS (5-95AB/1.5 min): RT=0.685 min, 499.0=[M+H]⁺, ESI⁺ showed 42% of desired product. The combined reaction solution was purified with reversed column ([Phenomenex luna C18]; mobile phase: [ACN] and [H₂O] (conditions: [water (0.1% FA)-ACN], B %: 85%-90%; Detector, UV 254 nm) and lyophilized to give the crude (50 mg), which LCMS (5-95AB/1.5 min): RT=0.683 min, 449.1=[M+H]⁺, ESI⁺ showed 92.6% of crude product. The crude product was further purified SFC (Column: OD 50×4.6 mm I.D., 3 um; Mobile phase: Phase A for CO₂, and Phase B for MeOH (0.05% DEA); Gradient elution: MeOH (0.05% DEA) in CO₂ from 5% to 40%; Flow rate: 3 mL/min; Detector: PDA; Column Temp: 35C; Back Pressure: 100 Bar). 2-[(2R,4S)-2-(1-cyclopropylpyrazol-4-yl)tetrahydropyran-4-yl]-6-(difluoromethyl)-4-(2,4-difluorophenyl)-7-methyl-pteridine (9.5 mg, 0.0188 mmol, 8.43% yield) was afforded as a yellow solid. LCMS: Rt: 0.654 min; [M+H]⁺=449.2; 99.0% purity at 220 nm and ¹H NMR (400 MHz, CDCl3) δ ppm 0.95-1.03 (m, 2H) 1.07-1.16 (m, 2H) 2.16-2.28 (m, 3H) 2.46 (br d, J=13.26 Hz, 1H) 3.06 (s, 3H) 3.52-3.65 (m, 2H) 3.76-3.89 (m, 1H) 4.28 (dt, J=11.26, 3.13 Hz, 1H) 4.57 (dd, J=11.38, 1.88 Hz, 1H) 6.58-6.91 (m, 1H) 6.99-7.06 (m, 1H) 7.12 (td, J=8.10, 2.06 Hz, 1H) 7.51 (d, J=2.75 Hz, 2H) 7.78 (td, J=8.16, 6.44 Hz, 1H).

### Synthesis of Compounds I-1333 and I-1334

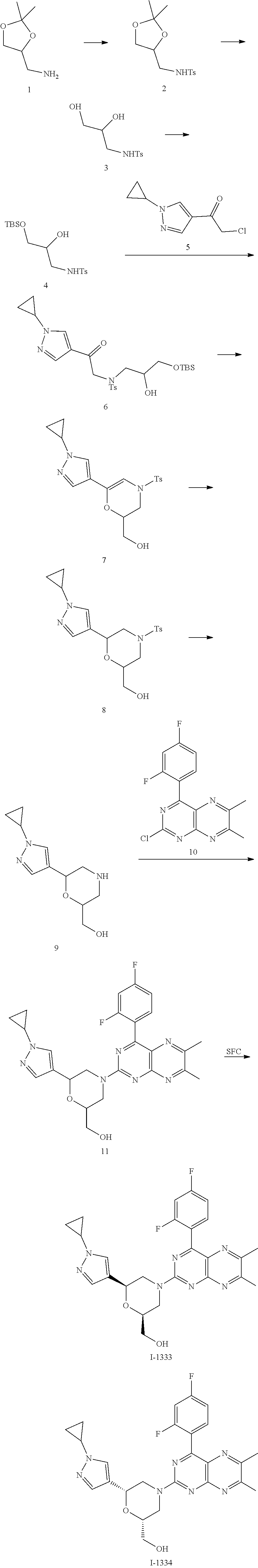

Step 1: To a solution of (2,2-dimethyl-1,3-dioxolan-4-yl)methanamine (1.00 eq, 1000 mg, 7.62 mmol) in DCM (30 mL) was added TEA (3.00 eq, 3.2 mL, 22.9 mmol). And then TsCl (1.20 eq, 1738 mg, 9.15 mmol) was added to the mixture at 0° C. The reaction was stirred for 12 h at 25° C. LCMS showed raw material was consumed completely and the major peak showed desired MS (M+H)⁺=286.1; purity=99.768% (220 nm). Retention time=0.816 min. The reaction was added water (50 mL) and then the organics were separated and dried (Na₂SO₄) before concentration to dryness to give N-[(2,2-dimethyl-1,3-dioxolan-4-yl)methyl]-4-methyl-benzenesulfonamide (2.00 g, 7.01 mmol, 91.93% yield) as white solid. LCMS: (M+H)⁺=286.1; purity=99.768% (220 nm). Retention time=0.816 min. 1H NMR (400 MHz, CDCl3) δ=7.77-7.73 (m, 2H), 7.35-7.30 (m, 2H), 4.79-4.70 (m, 1H), 4.22-4.16 (m, 1H), 4.03-3.97 (m, 1H), 3.72-3.66 (m, 1H), 3.18-3.11 (m, 1H), 3.01-2.93 (m, 1H), 2.49-2.40 (m, 3H), 1.38-1.34 (m, 3H), 1.33-1.30 (m, 3H).

Step 2: To a solution of N-[(2,2-dimethyl-1,3-dioxolan-4-yl)methyl]-4-methyl-benzenesulfonamide (1.00 eq, 1900 mg, 6.66 mmol) in HCl/MeOH (4 M, 22.8 eq, 38 mL, 152 mmol) was stirred for 1 h at 25° C. LCMS showed raw material was consumed and the major peak showed the desired MS (M+H)⁺=246.1; purity=96.23% (220 nm). Retention time=0.628 min. The reaction was concentrated in vacuo to give the crude residue N-(2,3-dihydroxypropyl)-4-methyl-benzenesulfonamide (1.60 g, 6.52 mmol, 97.96% yield) as red solid. MS (M+H)⁺=246.1; purity=96.23% (220 nm). Retention time=0.628 min.

Step 3: To a solution of N-(2,3-dihydroxypropyl)-4-methyl-benzenesulfonamide (1.00 eq, 1500 mg, 6.11 mmol) in THF (25 mL) was added imidazole (1.00 eq, 416 mg, 6.11 mmol) and then TBSCl (1.00 eq, 922 mg, 6.11 mmol) was added to the mixture at 0° C. and the mixture was stirred for 5 h at 25° C. LCMS showed the raw material was consumed most and the major peak showed desired MS (M+H)⁺=360.1; purity=65.12% (220 nm). Retention time=0.998 min). The reaction was combined with and added water (50 mL) and then extracted with EtOAc (30 mL*2) and the organics washed with 10 mL saturated brine solution. The organics were then separated and dried (Na₂SO₄) before concentration to dryness. The crude was then purified by silica gel column (PE/EA=2/1, Rf=0.4) to give N-[3-[tert-butyl(dimethyl)silyl]oxy-2-hydroxy-propyl]-4-methyl-benzenesulfonamide (1500 mg, 4.17 mmol, 68.22% yield) as a light-yellow solid. MS (M+H)⁺=360.1; purity=65.12% (220 nm). Retention time=0.998 min. 1H NMR (400 MHz, CDCl3) δ=7.77-7.72 (m, 2H), 7.34-7.28 (m, 2H), 5.16-4.98 (m, 1H), 3.77-3.69 (m, 1H), 3.63-3.49 (m, 2H), 3.16-3.07 (m, 1H), 2.97-2.87 (m, 1H), 2.65-2.54 (m, 1H), 2.48-2.35 (m, 3H), 0.95-0.80 (m, 9H), 0.09-0.01 (m, 6H).

Step 4: To a solution of N-[3-[tert-butyl(dimethyl)silyl]oxy-2-hydroxy-propyl]-4-methyl-benzenesulfonamide (1.00 eq, 1000 mg, 2.78 mmol) and 2-chloro-1-(1-cyclopropylpyrazol-4-yl)ethanone (1.10 eq, 565 mg, 3.06 mmol) in acetone (30 mL) was added K₂CO₃ (3.00 eq, 1153 mg, 8.34 mmol) and KI (1.00 eq, 462 mg, 2.78 mmol) and then the mixture was stirred for 12 h at 30° C. LCMS showed raw material was consumed and the major peak showed desired MS (M-18+H=490.2; purity=45.95%. Retention time=1.026 min. The reaction was added water (50 mL) and then extracted with EtOAc (20 mL*3) and the organics washed with 10 mL saturated brine solution. The organics were then separated and dried (Na₂SO₄) before concentration to dryness. The crude was then purified by silica gel column (PE/EA=1/1, Rf=0.5) to give N-[3-[tert-butyl(dimethyl)silyl]oxy-2-hydroxy-propyl]-N-[2-(1-cyclopropylpyrazol-4-yl)-2-oxo-ethyl]-4-methyl-benzenesulfonamide (600 mg, 1.18 mmol, 42.49% yield) as a light-yellow solid MS (M-18+H)⁺=490.2; purity=45.95% (220 nm). Retention time=1.026 min. ¹H NMR (400 MHz, CDCl3) δ=8.07-8.03 (m, 1H), 7.93-7.89 (m, 1H), 7.76-7.72 (m, 2H), 7.35-7.29 (m, 2H), 4.62-4.54 (m, 2H), 3.83-3.73 (m, 2H), 3.65 (tt, J=3.8, 7.4 Hz, 1H), 3.60-3.49 (m, 3H), 3.46-3.33 (m, 1H), 3.29-3.16 (m, 2H), 2.45-2.43 (m, 3H), 0.88-0.86 (m, 1H), 0.86-0.83 (m, 9H), 0.04-0.02 (m, 6H).

Step 5: To a solution of N-[2-(1-cyclopropylpyrazol-4-yl)-2-oxo-ethyl]-N-[2-hydroxy-3-[isopropyl(dimethyl)silyl]oxy-propyl]-4-methyl-benzenesulfonamide (1.00 eq, 780 mg, 1.58 mmol) in DCM (20 mL) was added triethylsilane (5.00 eq, 916 mg, 7.90 mmol) and trimethylsilyl trifluoromethanesulfonate (5.00 eq, 1.4 mL, 7.90 mmol) at 0° C. and then stirred for 16 h at 30° C. LCMS showed raw material was consumed and the major peak showed desired MS (M+H)⁺=376.1; purity=90.11% (220 nm). Retention time=0.837 min). The reaction was added water (20 mL) and then extracted with DCM (10 mL*2) and the organics washed with 10 mL saturated brine solution. The organics were then separated and dried (Na₂SO₄) before concentration to dryness to give 600 mg crude product and use for next step without purification. MS (M+H)⁺=376.1; purity=90.11% (220 nm). Retention time=0.837 min.

Step 6: To a solution of [6-(1-cyclopropylpyrazol-4-yl)-4-(p-tolylsulfonyl)morpholin-2-yl]methanol (1.00 eq, 200 mg, 0.530 mmol) in Methanol (10 mL) was added Mg (powder) (15.7 eq, 200 mg, 8.33 mmol) and Mg (chips) (15.7 eq, 200 mg, 8.33 mmol) at 25° C. and then the mixture was stirred for 16 h at 80° C. LCMS showed the most raw material was consumed and the major peak showed the desired MS (M+H)⁺=224.1; purity=92.29% (220 nm). Retention time=0.248 min). MeOH (20 mL) was added followed by filtration. The filtrate was concentrated in vacuo to give crude [6-(1-cyclopropylpyrazol-4-yl)morpholin-2-yl]methanol (200 mg, 0.896 mmol, 169.06% yield) as white solid. LCMS (M+H)⁺=224.1; purity=92.29% (220 nm). Retention time=0.248 min.

Step 7: To a solution of [6-(1-cyclopropylpyrazol-4-yl)morpholin-2-yl]methanol (1.00 eq, 200 mg, 0.896 mmol) in DMSO (2 mL) was added 2-chloro-4-(2,4-difluorophenyl)-6,7-dimethyl-pteridine (1.20 eq, 330 mg, 1.07 mmol) and DIEA (3.00 eq, 347 mg, 2.69 mmol) and then the mixture was stirred for 20 min at 100° C. LCMS showed raw material was consumed and the major peak showed desired MS (M+H)⁺=494.2; purity=60.85% (220 nm). Retention time=0.894 min). The reaction was added water 20 mL and then extracted with EtOAc (5 mL*3) and the organics washed with 5 mL saturated brine solution. The organics were then separated and dried (Na₂SO₄) before concentration to dryness. The crude was then purified by prep-HPLC (Phenomenex luna C18 150*25 mm*10 um, water (FA)-ACN)) and freeze-drying to give [6-(1-cyclopropylpyrazol-4-yl)-4-[4-(2,4-difluorophenyl)-6,7-dimethyl-pteridin-2-yl]morpholin-2-yl]methanol (80 mg, 0.162 mmol, 18.10% yield) as yellow solid. LCMS (M+H)⁺=494.2; purity=60.85% (220 nm). Retention time=0.894 min. ¹H NMR (400 MHz, CDCl3) δ=7.77-7.67 (m, 1H), 7.59-7.52 (m, 2H), 7.09-6.94 (m, 2H), 5.22-5.06 (m, 1H), 5.04-4.93 (m, 1H), 4.72-4.61 (m, 1H), 3.90-3.82 (m, 2H), 3.79-3.71 (m, 1H), 3.63-3.56 (m, 1H), 3.16-3.00 (m, 2H), 2.74-2.70 (m, 3H), 2.62-2.58 (m, 3H), 1.29-1.25 (m, 1H), 1.17-1.11 (m, 2H), 1.07-1.00 (m, 2H).

Step 8: The racemate product was purified by SFC (REGIS(S,S)WHELK-01 (250 mm * 25 mm, 10 um), MeOH-ACN) to give [(2S,6S)-6-(1-cyclopropylpyrazol-4-yl)-4-[4-(2,4-difluorophenyl)-6,7-dimethyl-pteridin-2-yl]morpholin-2-yl]methanol (39 mg, 0.0747 mmol, 46.09% yield) as yellow solid and [(2R,6R)-6-(1-cyclopropylpyrazol-4-yl)-4-[4-(2,4-difluorophenyl)-6,7-dimethyl-pteridin-2-yl]morpholin-2-yl]methanol (40 mg, 0.0774 mmol, 47.76% yield) as yellow solid. LCMS (M+H)⁺=494.2; purity=95.529% (220 nm). Retention time=0.881 min. ¹H NMR (400 MHz, CDCl3) δ ppm 1.00-1.07 (m, 2H)_{1.11}-1.17 (m, 2H)_{2.57}-2.64 (m, 3H)_{2.73} (s, 3H)_{3.01}-3.16 (m, 2H)_{3.56}-3.63 (m, 1H) 3.71-3.78 (m, 1H) 3.82-3.91 (m, 2H) 4.63-4.70 (m, 1H) 4.94-5.04 (m, 1H) 5.06-5.19 (m, 1H) 6.94-7.09 (m, 2H) 7.53-7.60 (m, 2H) 7.68-7.76 (m, 1H). LCMS (M+H)⁺=494.2; purity=96.555% (220 nm). Retention time=0.876 min. ¹H NMR (400 MHz, CDCl3) δ ppm 1.00-1.07 (m, 2H) 1.11-1.17 (m, 2H) 2.58-2.63 (m, 3H) 2.70-2.75 (m, 3H) 3.01-3.16 (m, 2H) 3.55-3.64 (m, 1H) 3.70-3.79 (m, 1H) 3.81-3.91 (m, 2H) 4.62-4.70 (m, 1H) 4.93-5.04 (m, 1H) 5.06-5.20 (m, 1H) 6.94-7.09 (m, 2H) 7.53-7.59 (m, 2H) 7.67-7.76 (m, 1H).

### Synthesis of Compound I-1338

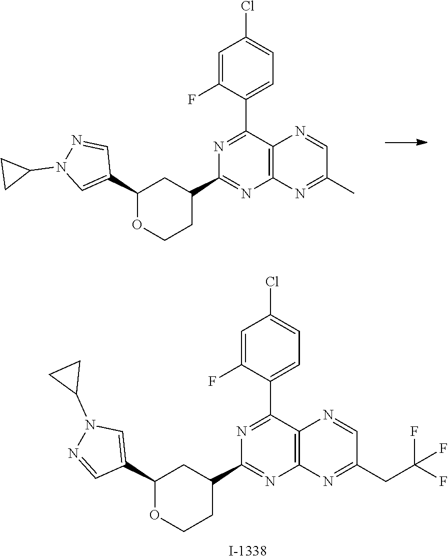

To a yellow solution of 4-(4-chloro-2-fluoro-phenyl)-7-methyl-2-[(2S,4R)-2-(1-cyclopropylpyrazol-4-yl)tetrahydropyran-4-yl]pteridine (1.00 eq, 100 mg, 0.215 mmol) in MeCN (2 mL) was added urea hydrogen peroxide adduct (2.00 eq, 40 mg, 0.430 mmol) at 0° C. to give a yellow solution. Then to the mixture was added TFAA (1.90 eq, 0.058 mL, 0.409 mmol) at 0° C. to give a yellow solution. The red solution was stirred at 15° C. for 12 h. LCMS showed the starting material was consumed completely and desired mass was detected. The reaction mixture was poured into saturated aqueous NaHCO₃ (20 mL) and stirred for 2 h. The resulting solution was extracted by DCM (20*3 mL) and the organic phase was dried and concentrated under vacuo to give a residue. The crude product was purified by prep-HPLC (FA, column: Phenomenex Luna C18 150*25 mm*10 um; mobile phase: [water (FA)-ACN]; B %: 39%-69%, 10 min) and lyophilized. 4-(4-chloro-2-fluoro-phenyl)-2-[(2S,4R)-2-(1-cyclopropylpyrazol-4-yl)tetrahydropyran-4-yl]-7-(2,2,2-trifluoroethyl)pteridine (14 mg, 0.0239 mmol, 11% yield) was obtained as yellow solid, LCMS [M+H]⁺=533.2; purity=94% (220 nm). Retention time=0.908 min. 1H NMR (400 MHz, CDCl3) δ=9.01 (s, 1H), 7.79-7.65 (m, 1H), 7.51 (d, J=2.3 Hz, 2H), 7.43-7.28 (m, 2H), 4.58 (dd, J=1.9, 11.4 Hz, 1H), 4.33-4.22 (m, 1H), 4.00 (q, J=10.3 Hz, 2H), 3.88-3.76 (m, 1H), 3.67-3.47 (m, 2H), 2.46 (d, J=13.2 Hz, 1H), 2.28-2.14 (m, 3H), 1.19-1.03 (m, 2H), 1.02-0.92 (m, 2H).

### Synthesis of Compound I-1343

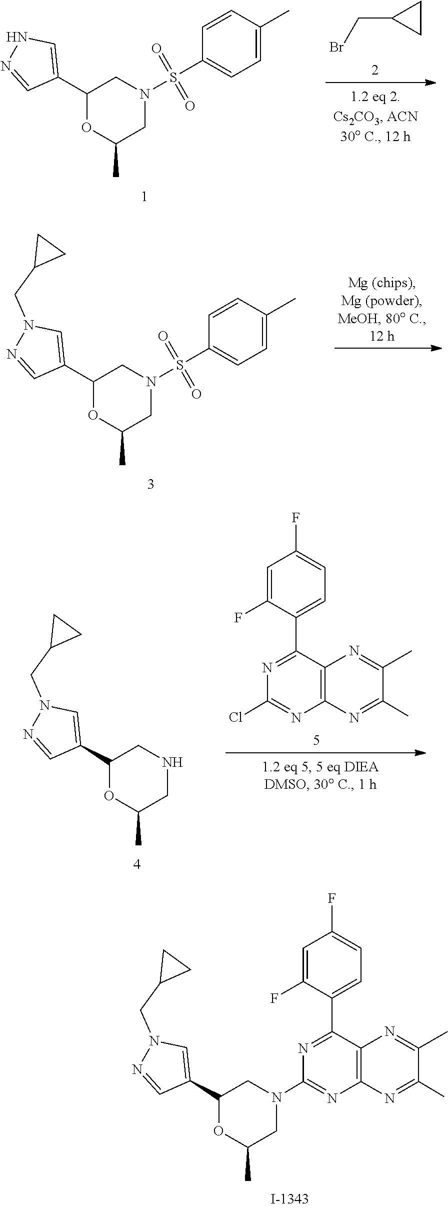

Step 1: A solution of (2R,6S)-2-methyl-4-(p-tolylsulfonyl)-6-(1H-pyrazol-4-yl)morpholine (1.00 eq, 150 mg, 0.467 mmol) and Cs₂CO₃ (3.00 eq, 455 mg, 1.40 mmol) in MeCN (0.5 mL) was added bromomethylcyclopropane (1.20 eq, 76 mg, 0.560 mmol) stirred at 30° C. for 2 hours. LCMS (5-95AB/1.5 min): RT=0.918 min, 376.3=[M+H]⁺, ESI⁺ showed 91% of desired product. The reaction mixture was filtered through celite, the filtrate was evaporated under reduced pressure to give the crude product, the crude product was for the next step without further workup. (M+H)⁺=376.3; purity=91% (220 nm). Retention time=0.918 min.

Step 2: To a solution of (6R)-2-[1-(cyclopropylmethyl)pyrazol-4-yl]-6-methyl-4-(p-tolylsulfonyl)morpholine (1.00 eq, 70 mg, 0.186 mmol) in Methanol (3 mL) was added Mg (powder) (32.3 eq, 144 mg, 6.02 mmol) and Mg (chips) (32.3 eq, 144 mg, 6.02 mmol) at 25° C. and then the mixture was stirred at 80° C. for 12 hours. LCMS (5-95AB/1.5 min): RT=0.689 min, 222.1=[M+H]⁺, ESI⁺ showed 88% of desired product. The reaction mixture was filtered through celite, the filtrate was evaporated under reduced pressure to give the crude product without further workup for the next step. (M+H)⁺=222.1; purity=88% (220 nm). Retention time=0.689 min.

Step 3: To a solution of (2S,6R)-2-[1-(cyclopropylmethyl)pyrazol-4-yl]-6-methyl-morpholine (1.00 eq, 40 mg, 0.181 mmol) and 2-chloro-4-(2,4-difluorophenyl)-6,7-dimethyl-pteridine (1.50 eq, 83 mg, 0.271 mmol) in DMSO (10 mL) was added DIEA (1.00 eq, 23 mg, 0.181 mmol), then the mixture was stirred at 100° C. for 20 min. LCMS (5-95AB/1.5 min): RT=0.986 min, 492.2=[M+H]⁺, ESI⁺ showed 68% of desired product. The reaction mixture was poured into H₂O (50 mL) and extracted with EtOAc (50 mL twice). The combined organic layers were washed with brine (50 mL), dried over Na₂SO₄ and concentrated in vacuo to give the residue. The residue was purified by prep-HPLC (Unisil 3-100 C18 Ultra 150*50 mm*3 um, water (FA)-ACN) to afford (2S,6R)-2-[1-(cyclopropylmethyl)pyrazol-4-yl]-4-[4-(2,4-difluorophenyl)-6,7-dimethyl-pteridin-2-yl]-6-methyl-morpholine 15 mg (mixed with impurity which shown in NMR). The product was purified by SFC (Column: Chiralpak AD-3 50×4.6 mm I.D., 3 um; Mobile phase: Phase A for C02, and Phase B for MeOH (0.05% DEA); Gradient elution: MeOH (0.05% DEA) in CO₂ from 5% to 40%; Flow rate: 3 mL/min; Detector: PDA; Column Temp: 35C; Back Pressure: 100 Bar”) and lyophilized to give (2S,6R)-2-[1-(cyclopropylmethyl)pyrazol-4-yl]-4-[4-(2,4-difluorophenyl)-6,7-dimethyl-pteridin-2-yl]-6-methyl-morpholine (2.4 mg, 0.00483 mmol, 2.67% yield) as yellow solid. (M+H)⁺=492.2; purity=68% (220 nm). Retention time=0.986 min. 1H NMR (400 MHz, CDCl3) δ=7.76-7.69 (m, 1H), 7.64-7.55 (m, 2H), 7.09-6.94 (m, 2H), 5.19-4.92 (m, 2H), 4.69-4.60 (m, 1H), 3.98 (br d, J=7.0 Hz, 2H), 3.89-3.81 (m, 1H), 3.12 (dd, J=11.2, 13.1 Hz, 1H), 2.90-2.82 (m, 1H), 2.72 (s, 3H), 2.60 (s, 3H), 1.34 (d, J=6.2 Hz, 3H), 1.31-1.26 (m, 1H), 0.70-0.65 (m, 2H), 0.40 (q, J=5.0 Hz, 2H).

### Synthesis of Compounds I-1348 and I-1406

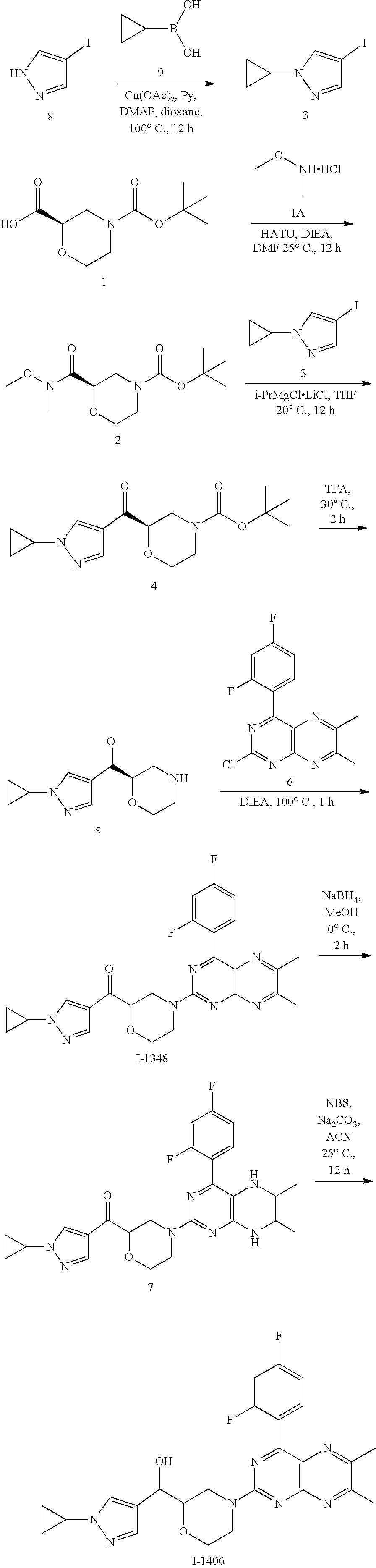

Step 1: To a solution of 4-iodo-1H-pyrazole (1.00 eq, 5.00 g, 25.8 mmol) in 1,4-Dioxane (150 mL) was added cyclopropylboronic acid (2.00 eq, 4.43 g, 51.6 mmol), Cu(OAc)₂ (1.00 eq, 5.15 g, 25.8 mmol), DMAP (4.00 eq, 12.58 g, 103 mmol) and Pyridine (2.50 eq, 50 mL, 64.4 mmol). The resulting mixture was stirred at 100° C. for 16 h under oxygen atmosphere. Color of the solution was changed from blue to black. LCMS showed the starting material was consumed completely and 97% desired MS (235.0 [M+1]⁺, ESI pos) was found. The reaction mixture was cooled to room temperature. The mixture was poured into water (500 mL) and extracted by ethyl acetate (3×500 mL), the combined organic layers were washed with brine, dried over anhydrous sodium sulfate, filtered and concentrated in vacuo. The solution was purified by column on silica (20 g SiO₂ cartridge, PE:EA=2:1, detection at 254 nm) and concentrated under reduced pressure to give 1-cyclopropyl-4-iodo-pyrazole (5.60 g, 22.7 mmol, 88.18% yield) as yellow oil. [M+H]⁺=235.0; purity=97% (220 nm). Retention time=0.763 min. ¹H NMR (400 MHz, CDCl3) δ=7.50 (s, 1H), 7.47 (s, 1H), 3.60 (tt, J=3.7, 7.3 Hz, 1H), 1.14-1.08 (m, 2H), 1.06-0.99 (m, 2H)

Step 2: To a colorless mixture of (2R)-4-tert-butoxycarbonylmorpholine-2-carboxylic acid (1.00 eq, 4.50 g, 19.5 mmol) in DMF (30 mL) was added DIEA (3.00 eq, 7.54 g, 58.4 mmol), HATU (1.20 eq, 8.88 g, 23.4 mmol), then the mixture was stirred at 15° C. for 10 min to give yellow solution, then N,O-dimethylhydroxylamine hydrochloride (1.10 eq, 2.09 g, 21.4 mmol) was added, then the mixture was stirred at 15° C. for 12 hr to give yellow solution. LCMS showed the starting material was consumed completely and 91% desired MS (219.2 [M-C₄H₈+H]⁺, ESI pos) was found. TLC (EA) showed the starting material was consumed completely and new spots observed. The mixture was poured into water (150 mL) and extracted by ethyl acetate (3×150 mL), the combined organic layers were washed with brine, dried over anhydrous sodium sulfate, filtered and concentrated in vacuo. The solution was purified by column on silica (10 g SiO₂ cartridge, PE:EA=1:1, detection at phosphomolybdic acid) and concentrated under reduced pressure to give tert-butyl (2R)-2-[methoxy(methyl)carbamoyl]morpholine-4-carboxylate (2.10 g, 7.66 mmol, 39.34% yield) as colorless oil. [M-C₄H₈+H]⁺=219.2; purity=91% (220 nm). Retention time=0.770 min. ¹H NMR (400 MHz, CDCl3) δ=4.35 (br d, J=4.3 Hz, 1H), 4.10-3.97 (m, 2H), 3.94-3.82 (m, 1H), 3.76 (s, 3H), 3.64-3.55 (m, 1H), 3.22 (s, 3H), 3.06 (br d, J=8.2 Hz, 2H), 1.48 (s, 9H).

Step 3: To a colorless mixture of 1-cyclopropyl-4-iodo-pyrazole (1.00 eq, 800 mg, 3.42 mmol) in THF (10 mL) was added iPrMgCl LiCl (1.20 eq, 3.2 mL, 4.10 mmol) at 20° C. The mixture was stirred at 20° C. for 1 hr. TLC showed 1-cyclopropyl-4-iodo-pyrazole (EA:PE=1:5, Rf=0.3) was consumed and a new spot (EA:PE=1:5, Rf=0.03) was detected. To the solution of tert-butyl (2R)-2-[methoxy(methyl)carbamoyl]morpholine-4-carboxylate (1.40 eq, 1313 mg, 4.79 mmol) in THF (4 mL) was added prepared Grignard reagent at 5° C., then the mixture was stirred at 20° C. for 2 hr under N₂ atmosphere. LC-MS showed that some of the Grignard reagent remained but the desired product (MS=266 [M-56+H]⁺, ESI, POS) was detected. The mixture was stirred at 20° C. for another 10 hrs. LC-MS showed the Grignard reagent was consumed and desired MS (MS=266.0 [M-56+H]⁺, ESI, POS) was detected. The reaction mixture was quenched with saturated NH₄Cl solution (50 mL) and extracted with EtOAc (50 mL×3). The organic phase washed with brine (50 mL). After concentration, the residue was purified by prep-TLC (EA:PE=1:1, Rf=0.3). Tert-butyl (2R)-2-(1-cyclopropylpyrazole-4-carbonyl)morpholine-4-carboxylate (820 mg, 2.16 mmol, 63.08% yield) was obtained. [M-56+H]⁺=266.0; purity=84.5% (220 nm). Retention time=0.849 min.

Step 4: To a colorless mixture of tert-butyl (2R)-2-(1-cyclopropylpyrazole-4-carbonyl)morpholine-4-carboxylate (1.00 eq, 100 mg, 0.311 mmol) in TFA (83.9 eq, 2.0 mL, 26.1 mmol), then the mixture was stirred at 30° C. for 2 hrs. LCMS showed the starting material was consumed completely and 54% desired MS (222.1 [M+H]⁺, ESI pos) was found. The pH was adjusted to 7 with sat. aq. NaHCO₃ (5 mL), and the mixture was extracted with DCM (20 mL×3). The combined organic layers were washed with brine, dried over anhydrous sodium sulfate, filtered and concentrated in vacuo. (1-cyclopropylpyrazol-4-yl)-[(2R)-morpholin-2-yl]methanone (80 mg, 0.210 mmol, 67.39% yield) was obtained as colorless oil. [M+H]⁺=222.1; purity=54% (220 nm).

Step 5: To a yellow mixture of (1-cyclopropylpyrazol-4-yl)-[(2R)-morpholin-2-yl]methanone (1.00 eq, 40 mg, 0.154 mmol) in DMSO (5 mL) was added 2-chloro-4-(2,4-difluorophenyl)-6,7-dimethyl-pteridine (1.00 eq, 55 mg, 0.154 mmol), DIPEA (3.00 eq, 0.080 mL, 0.461 mmol), then the mixture was stirred at 100° C. for 1 hr to give brown solution. LCMS showed the starting material was consumed completely and 73% desired MS (492.1 [M+H]⁺, ESI pos) was found. The combined mixture was cooled to room temperature. The mixture was purified by prep-HPLC (column: Waters Xbridge 150*25 mm*5 um; mobile phase: water (NH₄HCO₃)-ACN; B %: 45%-75%, 8 min), the purified solution was lyophilized to give (1-cyclopropylpyrazol-4-yl)-[4-[4-(2,4-difluorophenyl)-6,7-dimethyl-pteridin-2-yl]morpholin-2-yl]methanone (2.5 mg, 0.00505 mmol, 3% yield) as yellow solid. LCMS Rt: 0.947 min; m/z: 492.1 [M+H]⁺, 100% purity at 220 nm. ¹H NMR (400 MHz, CDCl3) δ=8.20 (s, 1H), 8.08 (s, 1H), 7.74 (dt, J=6.6, 8.1 Hz, 1H), 7.10-6.93 (m, 2H), 5.16 (br d, J=13.5 Hz, 1H), 4.87 (br d, J=13.5 Hz, 1H), 4.48 (br d, J=7.9 Hz, 1H), 4.25-4.15 (m, 1H), 3.83 (dt, J=2.7, 11.4 Hz, 1H), 3.65 (tt, J=3.7, 7.3 Hz, 1H), 3.47-3.32 (m, 2H), 2.73 (s, 3H), 2.61 (s, 3H), 1.23-1.15 (m, 2H), 1.13-1.06 (m, 2H).

Step 6: To a yellow mixture of (1-cyclopropylpyrazol-4-yl)-[(2R)-4-[4-(2,4-difluorophenyl)-6,7-dimethyl-pteridin-2-yl]morpholin-2-yl]methanone (1.00 eq, 20 mg, 0.0407 mmol) in Methanol (1 mL) was added NaBH₄ (5.00 eq, 7.7 mg, 0.203 mmol) at 0° C. under N₂ atmosphere, then the mixture was stirred at 0° C. for 2 hr under N₂ atmosphere. LCMS showed the starting material was consumed completely and 93% desired MS (498.0 [M+H]⁺, ESI pos) was found. The mixture was poured into NH₄Cl aqueous (5 mL) and extracted by ethyl acetate (20 mL×3), the combined organic layers were washed with brine, dried over anhydrous sodium sulfate, filtered and concentrated in vacuo. (1-cyclopropylpyrazol-4-yl)-[(2R)-4-[4-(2,4-difluorophenyl)-6,7-dimethyl-5,6,7,8-tetrahydropteridin-2-yl]morpholin-2-yl]methanol (20 mg, 0.0374 mmol, 91.87% yield) was obtained as yellow solid. [M+H]⁺=498.0; purity=93% (220 nm). Retention time=0.591 min.

Step 7: To a yellow mixture of (1-cyclopropylpyrazol-4-yl)-[4-[4-(2,4-difluorophenyl)-6,7-dimethyl-5,6,7,8-tetrahydropteridin-2-yl]morpholin-2-yl]methanol (1.00 eq, 20 mg, 0.0402 mmol) in MeCN (0.5000 mL) was added NBS (2.00 eq, 14 mg, 0.0804 mmol) and Na₂CO₃ (3.00 eq, 13 mg, 0.121 mmol), then the mixture was stirred at 15° C. for 12 hr to give yellow solution. LCMS showed the starting material was consumed completely and 49% desired MS (494.3 [M+H]⁺, ESI pos) was found. The mixture was poured into Na₂SO₃ (10 mL) and extracted by ethyl acetate (20 mL×3), the combined organic layers were washed with brine, dried over anhydrous sodium sulfate, filtered and concentrated in vacuo. The solution was purified by prep-HPLC (column: Waters Xbridge 150*25 mm*5 um; mobile phase: water (NH₄HCO₃)-ACN; B %: 38%-68%, 8 min), the purified solution was lyophilized to give (1-cyclopropylpyrazol-4-yl)-[4-[4-(2,4-difluorophenyl)-6, 7-dimethyl-pteridin-2-yl]morpholin-2-yl] methanol (2.2 mg, 0.00448 mmol, 11.14% yield) as yellow solid (racemic): LC-MS Rt: 0.864 min; m/z: [M+H]⁺=494.2, 100% purity at 220 nm. ¹H NMR (400 MHz, CDCl3) δ=7.74-7.63 (m, 1H), 7.52 (s, 2H), 7.04 (dt, J=2.4, 8.3 Hz, 1H), 7.00-6.91 (m, 1H), 4.89-4.81 (m, 2H), 4.80-4.62 (m, 1H), 4.20-4.06 (m, 1H), 3.86-3.65 (m, 2H), 3.58 (tt, J=3.7, 7.2 Hz, 1H), 3.35-3.18 (m, 1H), 3.16-2.98 (m, 1H), 2.71 (s, 3H), 2.59 (s, 3H), 1.11 (br d, J=2.8 Hz, 2H), 1.05-0.94 (m, 2H).

### Synthesis of Compound I-1351

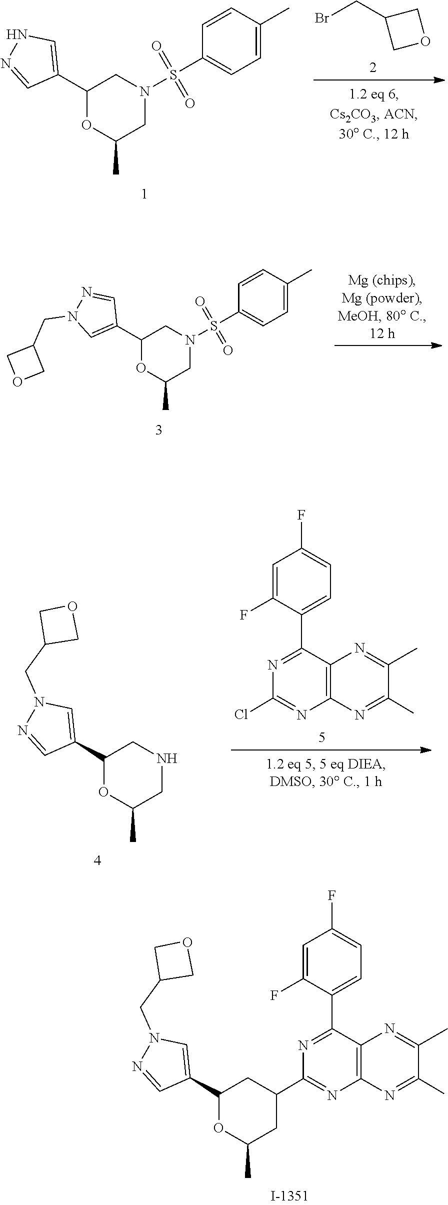

Step 1: solution of (2R,6S)-2-methyl-4-(p-tolylsulfonyl)-6-(1H-pyrazol-4-yl)morpholine (1.00 eq, 100 mg, 0.311 mmol) and Cs₂CO₃ (3.00 eq, 303 mg, 0.933 mmol) in MeCN (0.5 mL) was added 3-(bromomethyl)oxetane (1.20 eq, 56 mg, 0.373 mmol) stirred at 30° C. for 12 hours. LCMS (5-95AB/1.5 min): RT=0.858 min, 392.1=[M+H]⁺, ESI⁺ showed 71% of desired product. The reaction mixture was filtered, the filtrate was purified by prep-HPLC (Unisil 3-100 C18 Ultra 150*50 mm*3 um, water (FA)-ACN) to afford (2R,6S)-2-methyl-4-(p-tolylsulfonyl)-6-(1H-pyrazol-4-yl)morpholine (1.00 eq, 100 mg, 0.311 mmol) as white solid, which LCMS (M+H)⁺=376.3; purity=93% (220 nm). Retention time=0.854 min.

Step 2: To a solution of (2R,6S)-2-methyl-6-[1-(oxetan-3-ylmethyl)pyrazol-4-yl]-4-(p-tolylsulfonyl)morpholine (1.00 eq, 100 mg, 0.255 mmol) in Methanol (7 mL) was added Mg (chips) (16.3 eq, 100 mg, 4.17 mmol) and Mg (powder) (16.3 eq, 100 mg, 4.17 mmol) at 25° C. and then the mixture was stirred for 12 hours at 80° C. LCMS showed 64% of desired product (MS (238.8[M+H] *, ESI⁺, LC-RT: 0.287 min) was detected and 34% of starting material was remained. The reaction mixture was filtered through celite, the filtrate was evaporated under reduced pressure to give the crude product, which was then dissolved to Methanol (7 mL), Mg (chips) (16.3 eq, 100 mg, 4.17 mmol) and Mg (powder) (16.3 eq, 100 mg, 4.17 mmol) were added to the solution, purged with N₂ for 3 times and stirred at 80° C. for another 12 hours. LCMS showed 78% of desired product (MS (238.2 [M+H]⁺, ESI⁺, LC-RT: 0.274 min) was detected. The reaction mixture was filtered through celite, the filtrate was evaporated under reduced pressure to give the crude product without further workup for the next step. (M+H)⁺=238.8; purity=78% (220 nm). Retention time=0.283 min.

Step 3: To a solution of (2R,6S)-2-methyl-6-[1-(oxetan-3-ylmethyl)pyrazol-4-yl]morpholine (1.00 eq, 60 mg, 0.253 mmol) in DMSO (2.5 mL) was added 2-chloro-4-(2,4-difluorophenyl)-6,7-dimethyl-pteridine (1.50 eq, 116 mg, 0.379 mmol) and DIEA (5.00 eq, 163 mg, 1.26 mmol), then the mixture was stirred at 100° C. for 20 min. LCMS (5-95AB/1.5 min): RT=0.926 min, 508.3=[M+H]⁺, ESI⁺ showed 54% of desired product. The reaction mixture was poured into H₂O (50 mL) and extracted with EtOAc (50 mL) twice. The combined organic layers were washed with brine (50 mL), dried over Na₂SO₄ and concentrated in vacuo to give the residue. The residue was purified by prep-HPLC (Column: REGIS(S,S)WHELK-01 (250 mm*25 mm, 10 um), Condition: ACN/IPA (0.1% NH₃H₂O), Gradient Time (min): 3.7). The purified solution was lyophilized to afford the product 35 mg as yellow solid (mixed with impurity which shown in NMR). Then the residue was purified by prep-TLC (PE:EtOAc=0:1, Rf=0.65, UV) again to afford the product as yellow solid 27 mg (mixed with impurity which shown in NMR). Then the residue were separated by SFC (Column: Kromasil (S,S)Whelk-O1 50×4.6 mm I.D., 3.5 um, Mobile phase: Phase A for CO₂, and Phase B: IPA+CAN (0.05% DEA), 40% B in CO₂;Flow rate: 3 mL/min; Detector: PDA; Column Temp: 35C; Back Pressure: 100 Bar”) and lyophilized to give (2R,6S)-4-[4-(2,4-difluorophenyl)-6,7-dimethyl-pteridin-2-yl]-2-methyl-6-[1-(oxetan-3-ylmethyl)pyrazol-4-yl]morpholine (15 mg, 0.0261 mmol, 10.34% yield) as yellow solid. (M+H)+=508.3; purity=54% (220 nm). Retention time=0.926 min. 1H NMR (400 MHz, CDCl3) δ=7.72 (br d, J=6.6 Hz, 1H), 7.58 (s, 1H), 7.53 (s, 1H), 7.54-7.52 (m, 1H), 7.46 (s, 1H), 7.07-6.96 (m, 2H), 5.17-4.94 (m, 2H), 4.88-4.81 (m, 2H), 4.68-4.58 (m, 1H), 4.56-4.48 (m, 2H), 4.45-4.38 (m, 2H), 3.90-3.79 (m, 1H), 3.58-3.48 (m, 1H), 3.14-3.02 (m, 1H), 2.90-2.82 (m, 1H), 2.72 (s, 3H), 2.60 (s, 3H), 1.33 (d, J=6.2 Hz, 3H).

### Synthesis of Compound 1-1356

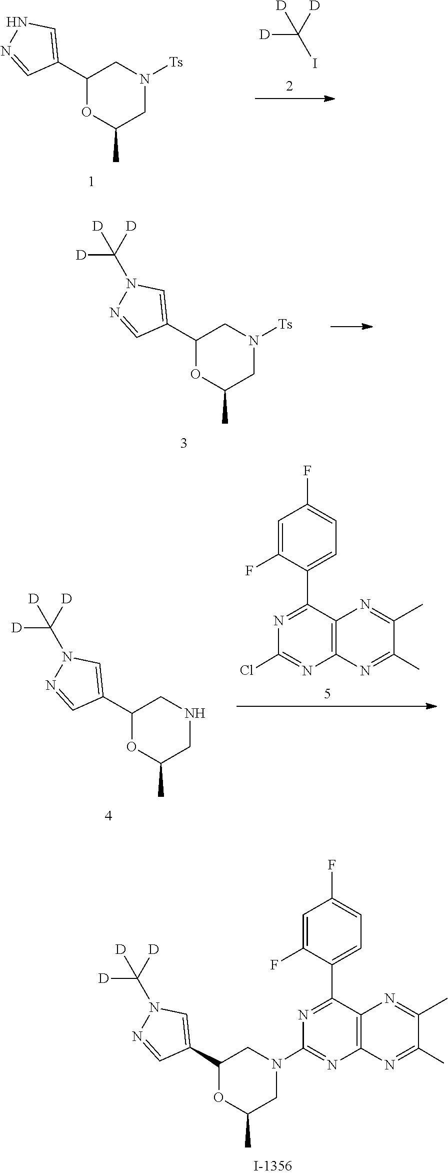

Step 1: To a solution of (2R,6S)-2-methyl-4-(p-tolylsulfonyl)-6-(1H-pyrazol-4-yl)morpholine (1.00 eq, 100 mg, 0.311 mmol) in DMF (1.5 mL) was added K₂CO₃ (2.00 eq, 86 mg, 0.622 mmol) and trideuterio(iodo)methane (1.20 eq, 54 mg, 0.373 mmol). The mixture was stirred at 30° C. for 12 h. LCMS showed one major peak with desired product was detected (82.5%, Rt: 0.863 min; [M+H]⁺=339.1 at 220 nm). The mixture was diluted with 5 mL H₂O, extracted with EA (10 mL*3), the combine organic layers was dried over anhydrous Na₂SO₄, filtered and concentrated under reduced pressure to give the (2R,6S)-2-methyl-4-(p-tolylsulfonyl)-6-[1-(trideuteriomethyl)pyrazol-4-yl]morpholine (90 mg, 0.266 mmol, 85.47% yield) as yellow solid and the mixture was used to the next step directly. [M+H]⁺=339.1; purity=82.5% (220 nm). Retention time=0.863 min.

Step 2: To a solution of (2R,6S)-2-methyl-4-(p-tolylsulfonyl)-6-[1-(trideuteriomethyl)pyrazol-4-yl]morpholine (1.00 eq, 80 mg, 0.236 mmol) in Methanol (16 mL) was added Mg (powder) (15.9 eq, 90 mg, 3.75 mmol) and Mg (chips) (15.9 eq, 90 mg, 3.75 mmol) at 25° C. and then the mixture was stirred for 16 h at 80° C. LCMS showed 60% of starting material was remained and one new peak was detected (no desired product mass signal). Mg (chips) (15.9 eq, 90 mg, 3.75 mmol) was added to the reaction mixture and then the mixture was stirred at 80° C. for another 12 h. LCMS showed the starting material was consumed completely and one new major peak was detected (no desired product mass signal). The mixture was filtered and washed with MeOH (20 mL*3), the combine organic layers was concentrated under reduced pressure to give crude (2R,6S)-2-methyl-6-[1-(trideuteriomethyl)pyrazol-4-yl]morpholine (88 mg, 0.478 mmol, 202.05% yield) as white solid and the residue was used to the next step directly. HPLC purity=84.8% (220 nm). Retention time=0.222 min.

Step 3: To a solution of 2-chloro-4-(2,4-difluorophenyl)-6,7-dimethyl-pteridine (1.00 eq, 85 mg, 0.277 mmol) in DMSO (2.5 mL) was added (2R,6S)-2-methyl-6-[1-(trideuteriomethyl)pyrazol-4-yl]morpholine (1.50 eq, 77 mg, 0.416 mmol) and DIPEA (4.00 eq, 0.19 mL, 1.11 mmol). The mixture was stirred at 100° C. for 20 min. LCMS showed the starting material was consumed completely and desired product was detected (32%, Rt: 0.932 min; [M+H]⁺=455.2 at 220 nm). The combined mixture was diluted with 20 mL H₂O, extracted with EA (30 mL*3), the combine organic layers was dried over anhydrous Na₂SO₄, filtered and concentrated under reduced pressure to give the residue. The residue was purified by column silica chromatography (PE/EtOAc=0-60%, PE/EtOAc=1/1, the desired product Rf=0.5) to give the crude product. The crude product was purified again by Prep-HPLC (Phenomenex Synergi C18 150*25 mm*10 um, water (FA)-ACN) and lyophilized to give the (2R,6S)-4-[4-(2,4-difluorophenyl)-6,7-dimethyl-pteridin-2-yl]-2-methyl-6-[1-(trideuteriomethyl)pyrazol-4-yl]morpholine (6.6 mg, 0.0143 mmol, 5.17% yield) as yellow solid, LCMS [M+H]⁺=455.2, purity=98.71% (220 nm). Retention time=0.929 min. 1H NMR (400 MHz, CDCl3) δ=7.76-7.68 (m, 1H), 7.56 (s, 1H), 7.46 (s, 1H), 7.08-7.02 (m, 1H), 7.01-6.94 (m, 1H), 5.19-4.91 (m, 2H), 4.63 (dd, J=2.3, 10.8 Hz, 1H), 3.84 (ddd, J=2.5, 6.3, 10.3 Hz, 1H), 3.09 (dd, J=11.0, 13.3 Hz, 1H), 2.85 (dd, J=10.8, 13.3 Hz, 1H), 2.72 (s, 3H), 2.60 (s, 3H), 1.33 (d, J=6.3 Hz, 3H). SFC showed 100% ee.

### Synthesis of Compounds I-1361

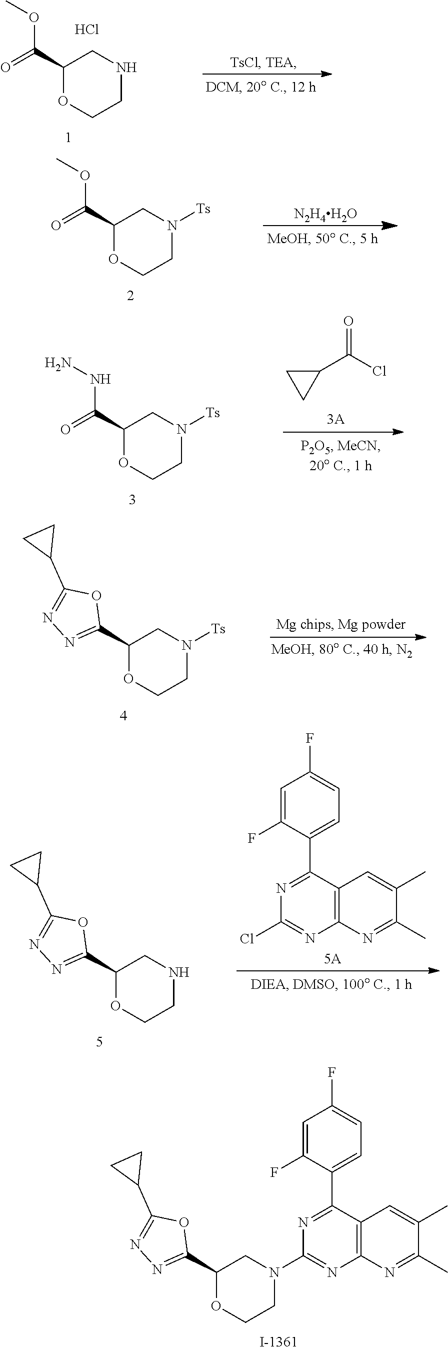

Step 1: To a solution of methyl (2R)-morpholine-2-carboxylate; hydrochloride (1.00 eq, 950 mg, 5.23 mmol) and TEA (3.00 eq, 2.2 mL, 15.7 mmol) in DCM (10 mL) was added TsCl (1.20 eq, 1391 mg, 6.28 mmol) at 0° C. The mixture was stirred at 25° C. for 12 hours. LCMS showed the starting material was consumed completely and a major peak with desired mass (98%, MS: 300.1 [M+H]⁺, ESI pos). The reaction mixture was partitioned between DCM (200 mL) and water (200 mL).The combined organic layer was dried over anhydrous sodium sulphate, concentrated under reduced pressure to get the crude residue. The residue was purified by column chromatography on silica gel (eluted with petroleum ether/ethyl acetate=100: 1 to 1: 1, R_{f}=0.6) to afford methyl (2R)-4-(p-tolylsulfonyl) morpholine-2-carboxylate (1.40 g, 4.68 mmol, 89.41% yield) as white solid. (M+H)⁺=300.1; purity=99% (220 nm). Retention time=0.806 min. ¹H NMR (400 MHz, CDCl3) δ ppm 2.44 (s, 3H) 2.52-2.64 (m, 2H) 3.43 (br dd, J=11.76, 1.50 Hz, 1H) 3.68-3.76 (m, 2H) 3.78 (s, 3H) 4.05 (dt, J=11.63, 3.00 Hz, 1H) 4.24 (dd, J=9.57, 3.06 Hz, 1H) 7.35 (d, J=8.13 Hz, 2H) 7.64 (d, J=8.25 Hz, 2H).

Step 2: To a solution of methyl (2R)-4-(p-tolylsulfonyl) morpholine-2-carboxylate (1.00 eq, 700 mg, 2.34 mmol) in Methanol (6 mL) was added hydrazine monohydrate (1.60 eq, 0.12 mL, 3.74 mmol) at 25° C. The reaction mixture was heated at 50° C. for 5 hours. LCMS showed the starting material was consumed completely and a major peak with desired mass (97%, MS: 300.1 [M+H]⁺, ESI pos). The resulting mixture was cooled to room temperature and poured into water (50 mL) then extracted with EtOAc (3*25 mL). The combined organic layer was dried over anhydrous sodium sulphate, concentrated under reduced pressure to afford crude (2R)-4-(p-tolylsulfonyl)morpholine-2-carbohydrazide (750 mg, 2.51 mmol, 107.14% yield) as colorless solid. (M+H)⁺=300.1; purity=97% (220 nm). Retention time=0.650 min. ¹H NMR (400 MHz, DMSO-d6) δ ppm 2.17 (t, J=10.88 Hz, 1H) 2.29 (td, J=11.38, 3.13 Hz, 1H) 2.42 (s, 3H) 3.17 (s, 1H) 3.41 (br s, 1H) 3.51-3.64 (m, 3H) 3.90 (br d, J=11.13 Hz, 1H) 4.02 (dd, J=10.13, 2.63 Hz, 1H) 4.15-4.47 (m, 1H) 7.48 (br d, J=8.00 Hz, 2H) 7.63 (d, J=8.13 Hz, 2H) 8.57-9.51 (m, 1H).

Step 3: To a solution of (2R)-4-(p-tolylsulfonyl) morpholine-2-carbohydrazide (1.00 eq, 750 mg, 2.51 mmol) and P₂O₅ (6.00 eq, 2134 mg, 15.0 mmol) in MeCN (20 mL) was added cyclopropanecarbonyl chloride (1.20 eq, 314 mg, 3.01 mmol) at room temperature. The reaction mixture was stirred at 50° C. for 1 hour. LCMS showed the starting material was consumed completely and a major peak with desired mass (91%, MS: 350.1 [M+H]⁺, ESI pos). The mixture was filtered through a celite pad, and the filtrate was concentrated to give the crude product, the crude product was used directly for the next step. (M+H)⁺=350.1; purity=87% (220 nm). Retention time=0.839 min.

Step 4: To a solution of (2R)-2-(5-cyclopropyl-1,3,4-oxadiazol-2-yl)-4-(p-tolylsulfonyl)morpholine (1.00 eq, 200 mg, 0.572 mmol) in Methanol (25 mL) was added Mg (powder) (10.0 eq, 137 mg, 5.72 mmol) and Mg (chips) (10.0 eq, 137 mg, 5.72 mmol) at 25° C. and then the mixture was stirred at 80° C. for 12 hours under N₂. LCMS showed the starting material was consumed completely but only 16% desired compound was detected (16%, MS: 196.1 [M+H]⁺, ESI pos). The reaction mixture was filtered by celite to afford crude product as white solid. (M+H)⁺=196.1; purity=16% (220 nm). Retention time=0.123 min.

Step 5: To a solution of 2-chloro-4-(2,4-difluorophenyl)-6,7-dimethyl-pyrido[2,3-d]pyrimidine (5.00 eq, 250 mg, 0.818 mmol) and (2R)-2-(5-cyclopropyl-1,3,4-oxadiazol-2-yl)morpholine (1.00 eq, 32 mg, 0.164 mmol) in DMSO (2 mL) was added DIEA (1.33 eq, 28 mg, 0.218 mmol). The mixture was stirred at 100° C. for 1 hour. LCMS showed the starting material was consumed completely and a major peak with desired product mass (Rt: 0.795 min, m/z: 465.2 [M+H]⁺, 43% purity at 220 nm). The reaction was filtered and the filtrate was purified by prep-HPLC (flow: 25 mL/min; gradient: from 29-59% water (0.1% FA)-ACN over 7 min; column: Unisil 3-100 C18 Ultra 150*25 mm*5 um) and lyophilized to afford (2R)-2-(5-cyclopropyl-1,3,4-oxadiazol-2-yl)-4-[4-(2,4-difluorophenyl)-6,7-dimethyl-pyrido[2,3-d]pyrimidin-2-yl]morpholine (4.1 mg, 0.00853 mmol, 5.22% yield) as yellow solid, which was LCMS (M+H)⁺=465.2; purity=100% (220 nm). Retention time=0.830 min. 1H NMR (400 MHz, CDCl3) δ ppm 1.12-1.19 (m, 4H) 2.12-2.21 (m, 1H) 2.35 (s, 3H) 2.70 (s, 3H) 3.40-3.51 (m, 1H) 3.63 (dd, J=13.38, 10.13 Hz, 1H) 3.80-3.89 (m, 1H) 4.12-4.18 (m, 1H) 4.79-4.91 (m, 2H) 5.13-5.21 (m, 1H) 6.97-7.14 (m, 2H) 7.52-7.61 (m, 2H).

### Synthesis of Compounds I-1364 and I-1365

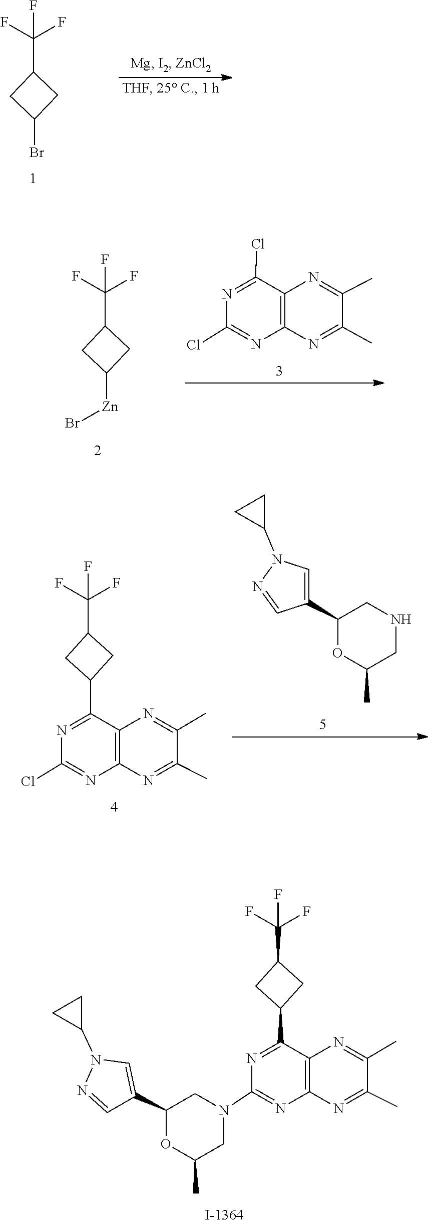

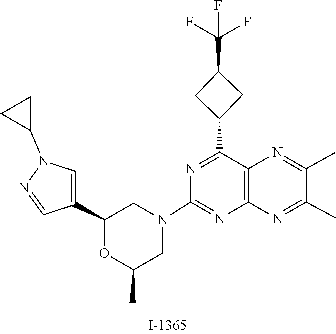 [SMILES: Cc1nc2nc(N3C[C@@H](C)O[C@@H](c4cnn(C5CC5)c4)C3)nc([C@H]3C[C@H](C(F)(F)F)C3)c2nc1C]

Step 1: Iodine (0.0500 eq, 6.3 mg, 0.0246 mmol) in THF (0.1 mL, extra dry) was added to a suspension of Mg (1.27 eq, 15 mg, 0.626 mmol) in dry THF (1.5 mL) under N₂ atmosphere, 1-bromo-3-(trifluoromethyl)cyclobutane (1.00 eq, 100 mg, 0.493 mmol) was added at 25° C. and heated until the yellow solution turned into colorless and then stirred at 25° C. for 1 h and a milky suspension was formed. ZnCl₂ (0.900 eq, 0.89 mL, 0.443 mmol) was added dropwise and the mixture was stirred for 30 minutes. A white precipitate formed. The reaction was used directly.

Step 2: A sealed bottle under N₂ atmosphere was charged with 2,4-dichloro-6,7-dimethyl-pteridine (1.00 eq, 25 mg, 0.109 mmol) and PdCl₂(Amphos) (0.0500 eq, 3.9 mg, 0.00546 mmol) and THF (1.5 mL) and purged with N₂ three times, chloro-[3-(trifluoromethyl)cyclobutyl]zinc (4.50 eq, 110 mg, 0.491 mmol) was added dropwise to the reaction solution at 25° C., then warmed to 40° C. and stirred for 1 hr. The reaction solution was changed from yellow to dark brown, the reaction was finished, LCMS showed the reactant was consumed and 71% of desired mass was detected, the reaction solution was poured into H₂O (5 mL), extracted with EtOAc (5 mL), dried over Na₂SO₄ and evaporated under reduced pressure to give the residue, which was then purified with Flash column (PE:EA=3:1, Rf=0.3) and dried in vacuo to give 2-chloro-6,7-dimethyl-4-[3-(trifluoromethyl)cyclobutyl]pteridine (18 mg, 0.0568 mmol, 52.08% yield) as yellow oil. [M+H]⁺=317.0; Retention time=0.930 min.

Step 3: A solution of 2-chloro-6,7-dimethyl-4-[3-(trifluoromethyl)cyclobutyl]pteridine (1.00 eq, 16 mg, 0.0505 mmol), (2S,6R)-2-(1-cyclopropylpyrazol-4-yl)-6-methyl-morpholine (1.00 eq, 10 mg, 0.0505 mmol) and DIEA (3.00 eq, 20 mg, 0.152 mmol) in DMSO (0.5 mL) was stirred at 100° C. for 1 h. LCMS showed the reactant was consumed and 50% of desired mass was detected, the reaction solution was poured into H₂O (5 mL), extracted with EtOAc (5 mL*3) and evaporated under reduced pressure to give the residue, which was then purified with Prep-HPLC (FA) and lyophilized to give (2S,6R)-2-(1-cyclopropylpyrazol-4-yl)-4-[6,7-dimethyl-4-[3-(trifluoromethyl)cyclobutyl]pteridin-2-yl]-6-methyl-morpholine (1.4 mg, 0.00288 mmol, 5.70% yield) as yellow solid and (2S,6R)-2-(1-cyclopropylpyrazol-4-yl)-4-[6,7-dimethyl-4-[3-(trifluoromethyl)cyclobutyl]pteridin-2-yl]-6-methyl-morpholine (2.7 mg, 0.00556 mmol, 11.00% yield) as yellow solid. [M+H]⁺=488.2; purity=98.1% (220 nm). Retention time=0.980 min. 1H NMR (400 MHz, CDCl3) δ=7.60-7.51 (m, 2H), 7.31-7.30 (m, 1H), 7.25-7.25 (m, 1H), 5.17-5.11 (m, 1H), 5.05-4.97 (m, 1H), 4.65-4.49 (m, 2H), 3.89-3.77 (m, 1H), 3.63-3.55 (m, 1H), 3.17-2.99 (m, 2H), 2.88-2.79 (m, 1H), 2.75-2.54 (m, 10H), 1.39-1.31 (m, 3H), 1.17-1.07 (m, 2H), 1.05-0.98 (m, 2H). [M+H]⁺=488.2; purity=100% (220 nm). Retention time=1.003 min. 1H NMR (400 MHz, CDCl3) δ=7.58-7.54 (m, 2H), 5.15-5.07 (m, 1H), 5.00 (br d, J=13.1 Hz, 1H), 4.73 (br t, J=8.3 Hz, 1H), 4.61 (dd, J=2.6, 10.9 Hz, 1H), 3.84 (ddd, J=2.6, 6.4, 10.2 Hz, 1H), 3.60 (td, J=3.5, 7.2 Hz, 1H), 3.08 (br t, J=11.7 Hz, 2H), 2.84 (br dd, J=11.1, 12.7 Hz, 1H), 2.75-2.64 (m, 7H), 2.63 (s, 3H), 1.35 (br d, J=6.0 Hz, 3H), 1.14 (br d, J=2.6 Hz, 2H), 1.03 (br d, J=5.3 Hz, 2H).

### Synthesis of Compounds I-1381 and I-1409

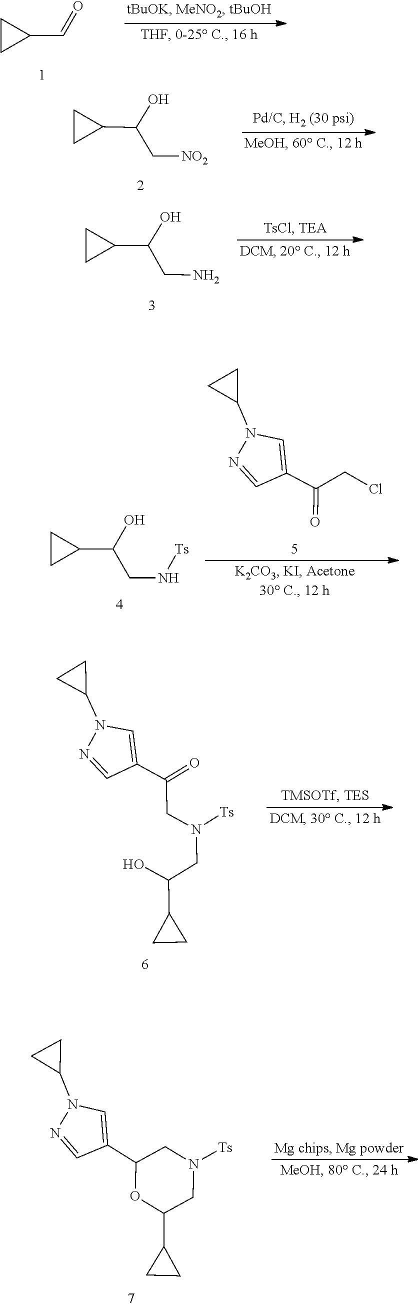

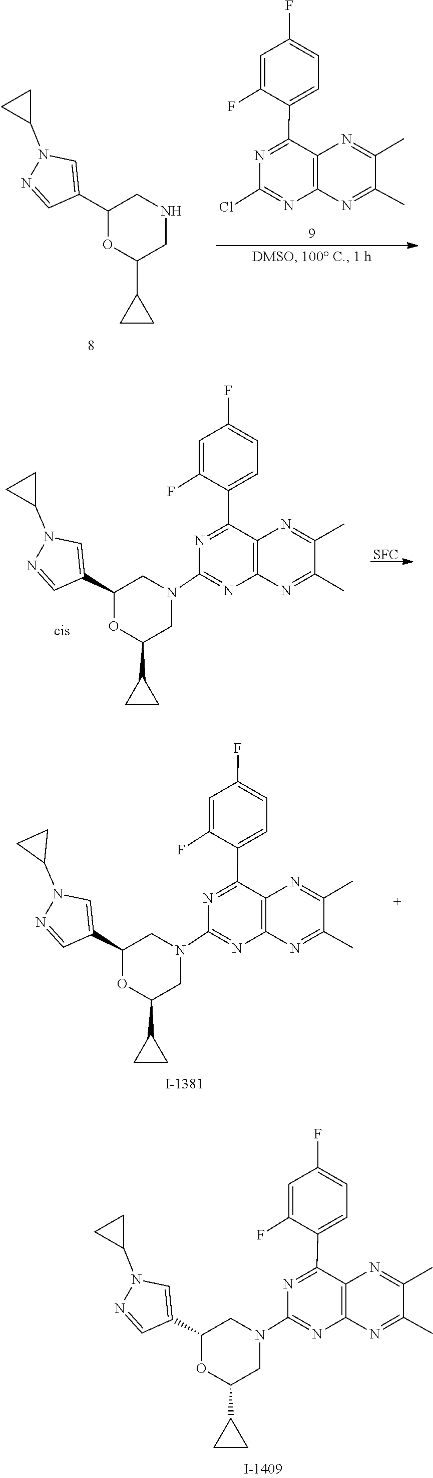

Step 1: To the mixture was added cyclopropanecarbaldehyde (1.00 eq, 5.4 mL, 71.3 mmol) and MeNO₂ (1.50 eq, 5.8 mL, 107 mmol). After stirring 5 minutes t-BuOK (0.200 eq, 14 mL, 14.3 mmol) was added slowly. During the addition, a white solid formed. The mixture was allowed to warm to 20° C. and stirred for 16 h. LCMS showed the starting material was consumed completely and one major peak was detected (98% at 220 nm, Rt: 0.307 min; no desired mass signal). The mixture was diluted with saturated aqueous NH₄Cl (150 mL) and extracted with DCM (50 mL×3). The combined organic layers were dried over Na₂SO₄ and filtered. The filtrate was concentrated under vacuum at 50° C. to afford crude 1-cyclopropyl-2-nitro-ethanol (9.50 g, 72.4 mmol, 101.56% yield) as yellow oil and H NMR which was used directly for the next step. ¹H NMR (400 MHz, CDCl₃) δ=4.60-4.49 (m, 2H), 3.68 (tt, J=4.1, 8.0 Hz, 1H), 2.45 (d, J=4.1 Hz, 1H), 0.96 (tq, J=4.8, 8.2 Hz, 1H), 0.71-0.57 (m, 2H), 0.54-0.43 (m, 1H), 0.40-0.30 (m, 1H).

Step 2: To the mixture of 1-cyclopropyl-2-nitro-ethanol (1.00 eq, 9.50 g, 72.4 mmol) in MeOH (100 mL) was added Pd/C (0.0124 eq, 0.95 g, 0.896 mmol) at 25° C. under N₂. Then the reaction mixture was stirred at 25° C. for 12 h under H₂ (30 psi). TLC (DCM/MeOH=10/1, the desired product R_{f}=0.4) indicated the starting material was consumed completely and one new spot formed. LCMS monitored the reaction (no desired mass signal). The reaction mixture was filtered and the filter was concentrated to give 2-amino-1-cyclopropyl-ethanol (7.80 g, 77.1 mmol, 106.44% yield) (crude) as yellow oil. ¹H NMR (400 MHz, CDCl₃) δ=3.27-3.14 (m, 1H), 2.97-2.92 (m, 1H), 2.91-2.69 (m, 1H), 0.93-0.79 (m, 1H), 0.60-0.44 (m, 2H), 0.43-0.17 (m, 2H). To the above mixture of 1-cyclopropyl-2-nitro-ethanol (1.00 eq, 7.00 g, 53.4 mmol) in MeOH (70 mL) was added Pd/C (0.0124 eq, 0.70 g, 0.660 mmol) at 25° C. under N₂, then the reaction mixture was stirred at 60° C. for 12 h under H₂ (30 Psi). TLC (DCM/MeOH=10/1, the desired product R_{f}=0.4) indicated the starting material was consumed completely and one new spot formed. LCMS monitored the reaction. The reaction mixture was filtered and the filter was concentrated to give crude 2-amino-1-cyclopropyl-ethanol (6.00 g, 59.3 mmol, 111.12% yield) as yellow oil which was used directly for the next step. ¹H NMR (400 MHz, CDCl₃) δ=2.95 (dd, J=3.3, 12.4 Hz, 1H), 2.84 (dt, J=3.4, 8.0 Hz, 1H), 2.77-2.67 (m, 1H), 0.89-0.79 (m, 1H), 0.58-0.45 (m, 2H), 0.39-0.30 (m, 1H), 0.26-0.17 (m, 1H).

Step 3: To the mixture of 2-amino-1-cyclopropyl-ethanol (1.00 eq, 0.50 g, 4.94 mmol) in DCM (5 mL) was added TEA (1.50 eq, 749 mg, 7.41 mmol), TsCl (1.10 eq, 1033 mg, 5.44 mmol) at 0° C., then the reaction mixture was stirred at 20° C. for 12 h. LCMS showed the starting material was consumed completely and one major peak with desired mass was detected (74%, Rt: 0.934 min; [M+H]⁺=238.1 at 220 nm). The reaction mixture was quenched by addition water (10 mL) at 20° C., and then diluted with EtOAc (20 mL) and extracted with EtOAc (20 mL×3). The combined organic layers were washed with sat. NaCl (aq., 10 mL), dried over drying Na₂SO₄, filtered and concentrated under reduced pressure to give a residue. The residue was purified by flash silica gel chromatography (ISCO®; 40 g SepaFlash® Silica Flash Column, Eluent of 0-50% EtOAc/PE; gradient @ 40 mL/min, PE/EtOAc=3/1, the desired product R_{f}=0.6) to give N-(2-cyclopropyl-2-hydroxy-ethyl)-4-methyl-benzenesulfonamide (1.15 g, 4.50 mmol, 91.12% yield) as colorless oil. LCMS: [M+H]⁺=238.1; purity=74.152% (220 nm). Retention time=0.934 min. ¹H NMR (400 MHz, CDCl₃) δ=7.76 (d, J=8.3 Hz, 2H), 7.32 (d, J=8.2 Hz, 2H), 4.97 (br s, 1H), 3.26-3.14 (m, 1H), 3.03-2.89 (m, 2H), 2.44 (s, 3H), 2.04-1.83 (m, 1H), 0.92-0.79 (m, 1H), 0.58-0.46 (m, 2H), 0.35-0.15 (m, 2H).

Step 4: A solution of N-(2-cyclopropyl-2-hydroxy-ethyl)-4-methyl-benzenesulfonamide (1.00 eq, 950 mg, 3.72 mmol) and K₂CO₃ (1.50 eq, 771 mg, 5.58 mmol), KI (1.00 eq, 618 mg, 3.72 mmol) in acetone (30 mL) was added 2-chloro-1-(1-cyclopropyl-1H-pyrazol-4-yl)ethan-1-one (1.30 eq, 893 mg, 4.84 mmol) at 0° C., then stirred at 30° C. for 12 h. LCMS and showed most of starting material was consumed and a major peak with desired mass (58.6%, Rt: 0.873 min; [M+Na]=426.1 at 220 nm). The reaction mixture was partitioned between ethyl acetate (50×2 mL) and water (100 mL). The combined organic layers were dried over Na₂SO₄, filtered and concentrated under reduced pressure to give a residue. The crude product was purified by column chromatography on silica gel eluted with PE/EtOAc from 1/0 to 0/1 (TLC:PE/EtOAc=1/1, the desired product R_{f}=0.2) to afford N-(2-cyclopropyl-2-hydroxy-ethyl)-N-[2-(1-cyclopropylpyrazol-4-yl)-2-oxo-ethyl]-4-methyl-benzenesulfonamide (1100 mg, 2.73 mmol, 73.27% yield) as a light-yellow oil, checked by LCMS. [M+H]⁺=403.1; purity=93% (220 nm). Retention time=0.558 min. ¹H NMR (400 MHz, CDCl₃) δ=8.05 (s, 1H), 7.91 (s, 1H), 7.74 (d, J=8.1 Hz, 2H), 7.32 (d, J=8.0 Hz, 2H), 4.67-4.57 (m, 1H), 4.48-4.37 (m, 1H), 3.65 (tt, J=3.7, 7.2 Hz, 1H), 3.42-3.36 (m, 1H), 3.33-3.26 (m, 1H), 3.09-2.97 (m, 1H), 2.44 (s, 3H), 1.17 (br d, J=4.4 Hz, 2H), 1.11 (br s, 2H), 1.08-0.97 (m, 1H), 0.81-0.72 (m, 1H), 0.53-0.41 (m, 2H), 0.33 (td, J=4.7, 9.0 Hz, 1H), 0.17 (qd, J=4.6, 9.2 Hz, 1H).

Step 5: To a solution of N-(2-cyclopropyl-2-hydroxy-ethyl)-N-[2-(1-cyclopropylpyrazol-4-yl)-2-oxo-ethyl]-4-methyl-benzenesulfonamide (1.00 eq, 1000 mg, 2.48 mmol) and TES (10.0 eq, 7.8 mL, 24.8 mmol) in DCM (20 mL), then TMSOTf (10.0 eq, 4.5 mL, 24.8 mmol) was added to the mixture at 0° C. The mixture was stirred at 30° C. for 12 h. LCMS showed the starting material was consumed completely and a major peak with desired mass (97%, Rt: 0.659 min; MS: 388.1 [M+H]⁺ at 220 nm). The reaction mixture was partitioned between EtOAc (50×2 mL) and water (100 mL).The combined organic layers were dried over Na₂SO₄, filtered and concentrated under reduced pressure to give a residue. The crude product was purified by column chromatography on silica gel (eluted with PE/EtOAc=1/0 to 0/1, TLC: PE/EtOAc=1/1, R_{f}=0.6) to afford 2-cyclopropyl-6-(1-cyclopropylpyrazol-4-yl)-4-(p-tolylsulfonyl)morpholine (530 mg, 1.37 mmol, 55.19% yield) as oil, checked by LCMS [M+H]⁺=388.3; purity=98.7% (220 nm). Retention time=0.867 min. ¹H NMR (400 MHz, DMSO-d₆) δ=7.75 (s, 1H), 7.66 (d, J=8.2 Hz, 2H), 7.47 (d, J=7.9 Hz, 2H), 7.36 (s, 1H), 4.47 (dd, J=2.3, 10.5 Hz, 1H), 3.70-3.50 (m, 3H), 3.07-2.95 (m, 1H), 2.42 (s, 3H), 2.10 (td, J=11.0, 16.6 Hz, 2H), 1.00-0.96 (m, 2H), 0.94-0.89 (m, 2H), 0.83-0.73 (m, 1H), 0.47-0.39 (m, 2H), 0.29 (br d, J=4.8 Hz, 2H).

Step 6: To the mixture of 2-cyclopropyl-6-(1-cyclopropylpyrazol-4-yl)-4-(p-tolylsulfonyl)morpholine (1.00 eq, 100 mg, 0.258 mmol) in MeOH (5 mL) was added Mg chips (10.0 eq, 62 mg, 2.58 mmol) and Mg powder (10.0 eq, 62 mg, 2.58 mmol) at 25° C. and the reaction mixture was stirred for 12 hours at 80° C. LCMS showed some starting material remained. Mg chips (10.0 eq, 62 mg, 2.58 mmol) was added to the mixture and the reaction mixture was stirred for 12 hours at 80° C. LCMS showed the starting material was consumed completely and a major peak with desired MS (92%, Rt: 0.248 min; MS: 234.2 [M+H]⁺ at 220 nm). The reaction mixture was concentrated under reduced pressure to give 2-cyclopropyl-6-(1-cyclopropylpyrazol-4-yl)morpholine (45 mg, 0.193 mmol, 74.74% yield) as white solid, which was used directly for the next step. [M+H]⁺=234.2; purity=92.637% (220 nm). Retention time=0.248 min.

Step 7: To a solution of 2-chloro-4-(2,4-difluorophenyl)-6,7-dimethyl-pteridine (1.00 eq, 60 mg, 0.196 mmol) and DIEA (3.00 eq, 0.097 mL, 0.587 mmol) in DMSO (2 mL) was added (2R,6S)-2-cyclopropyl-6-(1-cyclopropylpyrazol-4-yl)morpholine (1.00 eq, 46 mg, 0.196 mmol) (cis) at 25° C. Then the reaction mixture was stirred at 100° C. for 1 h. LCMS showed the starting material was consumed completely and the desired mass was detected (60%, Rt: 0.845 min; [M+H]⁺=504.4 at 220 nm). The reaction mixture was quenched by addition NH₄Cl (20 mL) at 0° C., and then extracted with EtOAc (15 mL×3). The combined organic layers were washed with brine (10 mL), dried over Na₂SO₄, filtered and concentrated under reduced pressure to give a residue. The residue was purified by prep-HPLC (column: Phenomenex luna C18 150×25 mm×10 um; mobile phase: [water (FA)-ACN]; B %: 56%-86%, 12 min) and lyophilized to give (2R,6S)-2-cyclopropyl-6-(1-cyclopropylpyrazol-4-yl)-4-[4-(2,4-difluorophenyl)-6,7-dimethyl-pteridin-2-yl]morpholine (25 mg, 0.0487 mmol, 24.87% yield) (cis) as yellow solid. LCMS, HPLC and SFC (Column: Chiralpak IC-3 50×4.6 mm I.D., 3 um; Mobile phase: Phase A for CO₂, and Phase B for IPA+ACN (0.05% DEA); Gradient elution: 40% IPA+ACN (0.05% DEA) in CO₂; Flow rate: 3 mL/min; Detector: PDA; Column Temp: 35C; Back Pressure: 100 Bar). (cis) [M+H]⁺=504.4; purity=98% (220 nm). Retention time=0.841 min. SFC peak 1 Retention time=1.306 min, peak 2 Retention time=1.939 min; peak 1/peak 2=1:1. ¹H NMR (400 MHz, CDCl₃) δ=7.64 (q, J=7.8 Hz, 1H), 7.47 (s, 2H), 7.04-6.84 (m, 2H), 4.98 (br d, J=9.0 Hz, 2H), 4.45 (dd, J=2.0, 10.8 Hz, 1H), 3.50 (tt, J=3.6, 7.3 Hz, 1H), 3.08-2.88 (m, 3H), 2.64 (s, 3H), 2.52 (s, 3H), 1.11-1.00 (m, 2H), 0.98-0.85 (m, 3H), 0.60-0.47 (m, 2H), 0.45-0.27 (m, 2H).

Step 8: A mixture of 2R,6S)-2-cyclopropyl-6-(1-cyclopropylpyrazol-4-yl)-4-[4-(2,4-difluorophenyl)-6,7-dimethyl-pteridin-2-yl]morpholine and (2S,6R)-2-cyclopropyl-6-(1-cyclopropylpyrazol-4-yl)-4-[4-(2,4-difluorophenyl)-6,7-dimethyl-pteridin-2-yl]morpholine. (2R,6S)-2-cyclopropyl-6-(1-cyclopropylpyrazol-4-yl)-4-[4-(2,4-difluorophenyl)-6,7-dimethyl-pteridin-2-yl]morpholine (1.00 eq, 20 mg, 0.0397 mmol) (cis) was purified by SFC (Column: DAICEL CHIRALPAK IC (250 mm×30 mm, 10 um); Mobile phase: Phase A for CO₂, and Phase B for IPA+CAN (0.1% NH₃H₂O IPA); Gradient elution: 50% IPA+ACN (0.1% NH₃H₂O IPA) in CO₂; Flow rate: 70 mL/min; Detector: PDA; Column Temp: 35C; Gradient Time: 4.2 min) to give (2R,6S)-2-cyclopropyl-6-(1-cyclopropylpyrazol-4-yl)-4-[4-(2,4-difluorophenyl)-6,7-dimethyl-pteridin-2-yl]morpholine (7.5 mg, 0.0149 mmol, 37.50% yield) as yellow solid and (2S,6R)-2-cyclopropyl-6-(1-cyclopropylpyrazol-4-yl)-4-[4-(2,4-difluorophenyl)-6,7-dimethyl-pteridin-2-yl]morpholine (8.8 mg, 0.0175 mmol, 44.00% yield) as yellow solid. LCMS, [M+H]⁺=504.3; purity=100% (220 nm). Retention time=0.993 min, ee=99.97%. ¹H NMR (400 MHz, CDCl₃) δ=7.77-7.67 (m, 1H), 7.55 (s, 2H), 7.09-6.93 (m, 2H), 5.06 (br d, J=10.1 Hz, 2H), 4.53 (dd, J=2.1, 10.8 Hz, 1H), 3.68-3.52 (m, 1H), 3.14-2.96 (m, 3H), 2.72 (s, 3H), 2.60 (s, 3H), 1.15-1.08 (m, 2H), 1.06-0.96 (m, 3H), 0.67-0.55 (m, 2H), 0.52-0.36 (m, 2H). Peak 2 in SFC: [M+H]⁺=504.3; purity=100% (220 nm). Retention time=0.990 min, ee=99.24%. ¹H NMR (400 MHz, CDCl₃) δ=7.76-7.66 (m, 1H), 7.55 (s, 2H), 7.09-6.93 (m, 2H), 5.06 (br d, J=10.6 Hz, 2H), 4.53 (br d, J=9.4 Hz, 1H), 3.58 (td, J=3.4, 6.8 Hz, 1H), 3.15-2.96 (m, 3H), 2.72 (s, 3H), 2.60 (s, 3H), 1.17-1.08 (m, 2H), 1.06-0.96 (m, 3H), 0.68-0.55 (m, 2H), 0.52-0.36 (m, 2H).

### Synthesis of I-1391 (Same general method as I-1588 and I-1396)

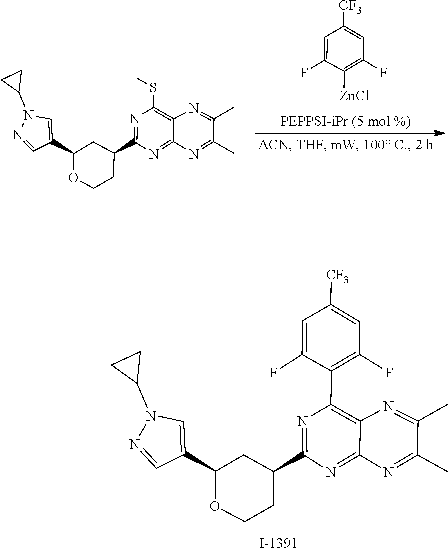

A flame-dried 10 ml microwave vial under N₂ was charged with PEPPSI™-SIPr (0.05 eq, 4.3 mg, 0.006 mmol) and chloro-[2,6-difluoro-4-(trifluoromethyl)phenyl]zinc (4.00 eq, 3.9 mL, 0.5 mmol). The mixture was then cooled to 0° C. MeCN (0.42 mL) was added followed by 2-[(2R,4S)-2-(1-cyclopropylpyrazol-4-yl)tetrahydropyran-4-yl]-6,7-dimethyl-4-methylsulfanyl-pteridine (1.00 eq, 50 mg, 0.13 mmol). The resulting mixture was purged with N₂ and irradiated under constant microwave for 2 h with the reaction temperature controlled at 100° C. The reaction mixture was diluted with water (20 mL) and EtOAc (20 mL). The aqueous layer was collected, and the organic layer was washed with water (2×30 mL). The organic phase was collected, dried over Na₂SO₄, filtered through a pad of Celite and concentrated under reduced pressure. The resulting material was purified by flash chromatography on a 10 g Biotage column using as gradient from 40-100% EtOAc in Hexanes followed by a wash using 0 to 20% MeOH in DCM. The desired fractions were evaporated to afford the desired analog as a mixture of diastereoisomers. The desired cis diastereoisomer was isolated by Prep-HPLC purification (Gemini® 5 um NX-C18 110 Å, 100×30 mm) using aqueous 10 mM ammonium formate (PH=3.8) and ACN (45-65%). 4-[2,6-difluoro-4-(trifluoromethyl)phenyl]-6,7-dimethyl-2-[(2R,4S)-2-(1-cyclopropylpyrazol-4-yl)tetrahydropyran-4-yl]pteridine (25 mg, 0.0471 mmol, 37% yield) as a yellow solid. ESI-MS (m/z+): 531.1 [M+H]⁺. ¹H NMR (CHCl₃-d, 400 MHz): ^{6H} 7.48 (2H, d, J=2.0 Hz), 7.35 (2H, d, J=7.5 Hz), 4.53 (1H, dd, J=11.4, 2.1 Hz), 4.24 (1H, d, J=11.5 Hz), 3.78 (1H, s), 3.53 (2H, td, J=7.3, 3.7 Hz), 2.84 (3H, s), 2.69 (3H, s), 2.40 (1H, s), 2.13-2.22 (3H, m), 1.05-1.08 (2H, m), 0.95-0.99 (2H, m).

### Synthesis of Compound I-1401

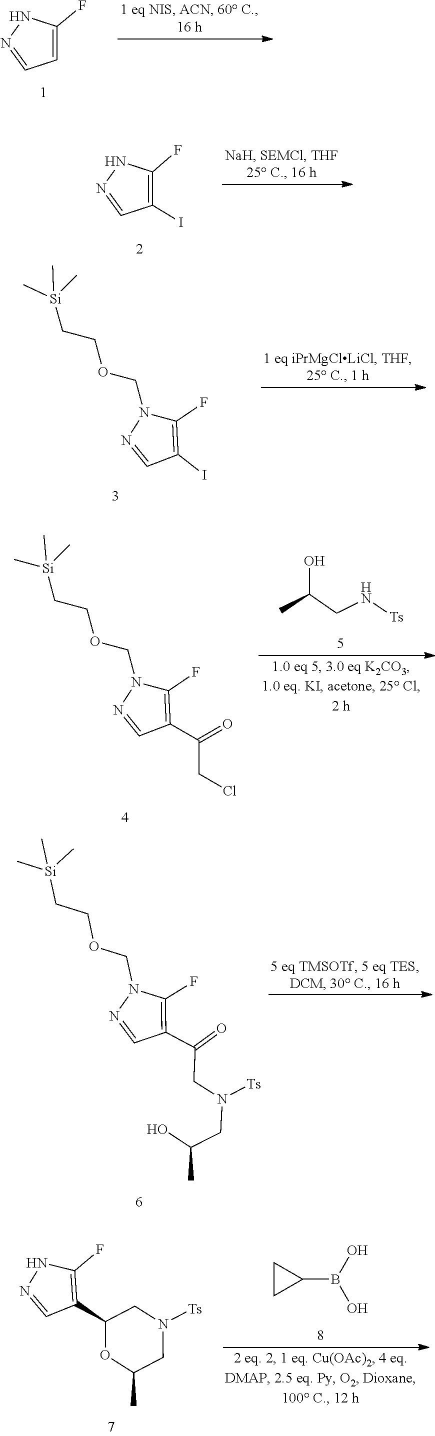

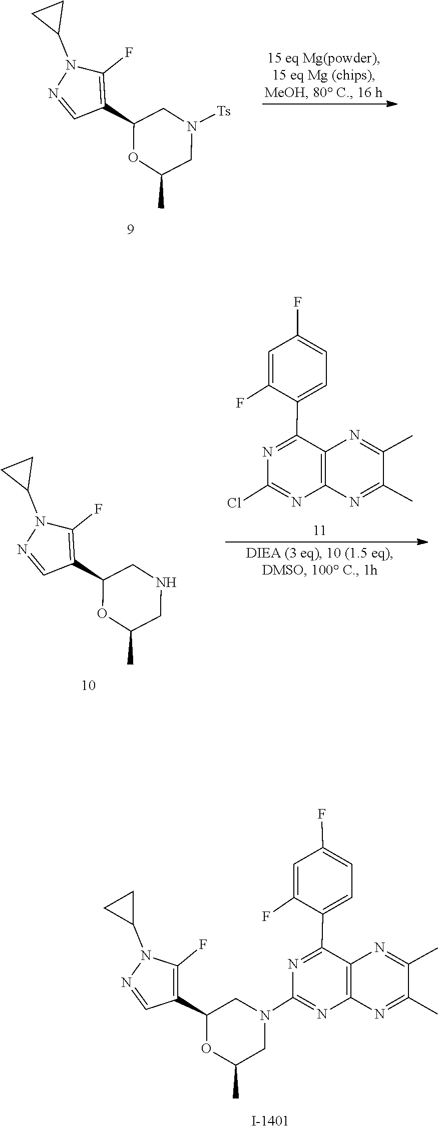 [SMILES: Cc1ccc(S(=O)(=O)N2C[C@@H](C)O[C@@H](c3cnn(C4CC4)c3F)C2)cc1 ; C[C@@H]1CNC[C@H](c2cnn(C3CC3)c2F)O1 ; Cc1nc2nc(Cl)nc(-c3ccc(F)cc3F)c2nc1C ; Cc1nc2nc(N3C[C@@H](C)O[C@@H](c4cnn(C5CC5)c4F)C3)nc(-c3ccc(F)cc3F)c2nc1C]

Step 1: A mixture of 5-fluoro-1H-pyrazole (1.00 eq, 900 mg, 10.5 mmol) in MeCN (10 mL) was added NIS (1.00 eq, 2353 mg, 10.5 mmol) at 25° C. Then mixture was stirred at 60° C. for 16 hours. TLC (PE:EA=3:1, new spot R_{f}=0.5) indicated stating material was consumed completely and one major new spot formed. The reaction mixture was diluted with water 20 mL and extracted with EA (50 mL*2). The combined organic layers were washed with aq. Na₂SO₃ (100 mL*2), dried over [Na₂SO₄], filtered and concentrated under reduced pressure to give a residue. The residue was purified by flash silica gel chromatography (Eluent of 0-15% Ethyl acetate/Petroleum ether) to give 5-fluoro-4-iodo-1H-pyrazole (1300 mg, 6.13 mmol, 58.65% yield) as yellow solid. ¹H NMR (400 MHz, CDCl₃) δ ppm 7.48 (d, J=1.38 Hz, 1H) 10.05-10.58 (m, 1H) 10.06-10.06 (m, 1H).

Step 2: A solution of 5-fluoro-4-iodo-1H-pyrazole (1.00 eq, 1300 mg, 6.13 mmol) in THF (20 mL) was purged with N₂ for 3 times at 0° C., NaH (1.20 eq, 294 mg, 7.36 mmol) was added slowly with temperature kept at 0° C. and stirred for 0.5 hour, then SEMCl (1.50 eq, 1.6 mL, 9.20 mmol) was added and warmed to 25° C. and stirred for 12 hours. TLC (PE/EA=5/1, new spot R_{f}=0.7) indicated starting material was consumed completely and one new spot formed. The reaction mixture was poured into saturated aqueous NH₄Cl (50 mL) slowly then the mixture was extracted with EA (80 mL*3). The organic layer was washed with brine, dried by Na₂SO₄. The solution was concentrated to give the residue. The crude product was purified by flash silica gel chromatography (Eluent of 0-15% Ethyl acetate/Petroleum ether) to give 2-[(5-fluoro-4-iodo-pyrazol-1-yl)methoxy]ethyl-trimethyl-silane (2100 mg, 5.83 mmol, 95.05% yield) was obtained as colorless oil. ¹H NMR (400 MHz, CDCl₃) δ ppm 0.00 (d, J=0.88 Hz, 9H) 0.88-0.94 (m, 2H) 3.55-3.61 (m, 2H) 5.26 (s, 2H) 7.45 (d, J=2.00 Hz, 1H).

Step 3: To a solution of 2-[(5-fluoro-4-iodo-pyrazol-1-yl)methoxy]ethyl-trimethyl-silane (1.00 eq, 1900 mg, 4.44 mmol) in THF (30 mL) was added iPrMgC·LiCl (1.00 eq, 3.4 mL, 4.44 mmol) at −78° C. and stirred for 30 mins, then 2-chloro-N-methoxy-N-methylacetamide (1.10 eq, 672 mg, 4.89 mmol) was added at 0° C. and stirred at 25° C. for 1 hour. TLC (PE/EA=5/1, starting material R_{f}=0.7, new spot R_{f}=0.3) indicated starting material was consumed completely and one new spot formed. The reaction mixture was poured into saturated aqueous NH₄Cl (100 mL) slowly and stirred at 0° C. for 10 mins, then the mixture was extracted with EtOAc (100 mL*3). The organic layer was washed with brine, dried by Na₂SO₄. The solution was concentrated to give the residue. The residue was purified by flash silica gel chromatography (Eluent of 0-13% Ethyl acetate/Petroleum ether) to give 2-chloro-1-[5-fluoro-1-(2-trimethylsilylethoxymethyl)pyrazol-4-yl]ethanone (950 mg, 3.08 mmol, 69.39% yield) as a yellow oil. 1H NMR (400 MHz, CDCl₃) δ ppm −0.01-0.02 (m, 9H) 0.92-0.97 (m, 2H) 3.61-3.66 (m, 2H) 4.48 (s, 2H) 5.31 (s, 2H) 8.04 (d, J=1.50 Hz, 1H).

Step 4: To a solution of 2-chloro-1-[5-fluoro-1-(2-trimethylsilylethoxymethyl)pyrazol-4-yl]ethanone (1.00 eq, 900 mg, 3.07 mmol) in Acetone (20 mL) was added N-[(2R)-2-hydroxypropyl]-4-methyl-benzenesulfonamide (1.20 eq, 846 mg, 3.69 mmol), K₂CO₃ (3.00 eq, 1274 mg, 9.22 mmol) and KI (1.00 eq, 510 mg, 3.07 mmol) and the solution was stirred for 16 hours at 30° C. LCMS (5-95AB/1.5 min): RT=0.834 min, 486.2=[M+H]⁺, ESI⁺ showed desired mass was detected. The residue was purified by flash silica gel chromatography (Eluent of 0-17% Ethyl acetate/Petroleum ether) to give N-[2-[5-fluoro-1-(2-trimethylsilylethoxymethyl)pyrazol-4-yl]-2-oxo-ethyl]-N-[(2R)-2-hydroxypropyl]-4-methyl-benzenesulfonamide (800 mg, 1.65 mmol, 53.59% yield)(crude) as a yellow oil. ¹H NMR (400 MHz, CDCl₃) δ ppm 0.02 (s, 9H) 0.93-0.98 (m, 2H) 1.13 (br d, J=6.13 Hz, 3H) 2.45 (s, 3H) 3.55-3.66 (m, 3H) 3.77-3.95 (m, 2H) 4.34-4.40 (m, 1H) 4.58-4.64 (m, 1H) 5.21 (s, 1H) 5.30 (s, 2H) 7.34 (br d, J=7.63 Hz, 2H) 7.64 (br d, J=7.88 Hz, 1H) 7.75 (br d, J=8.00 Hz, 2H).

Step 5: To a solution of N-[2-[5-fluoro-1-(2-trimethylsilylethoxymethyl)pyrazol-4-yl]-2-oxo-ethyl]-N-[(2R)-2-hydroxypropyl]-4-methyl-benzenesulfonamide (1.00 eq, 800 mg, 1.65 mmol) in DCM (16 mL) was added triethylsilane (5.00 eq, 955 mg, 8.24 mmol) and trimethylsilyl trifluoromethanesulfonate (5.00 eq, 1.5 mL, 8.24 mmol) at 0° C. and then stirred for 16 hours at 25° C. LCMS (5-95AB/1.5 min): RT=0.853 min, 340.1=[M+H]⁺, ESI⁺ showed starting material was consumed completely and one major peak with desired mass was detected. TLC (EA, new spot R_{f}=0.4) indicated reactant was consumed completely and one new spot formed. The reaction mixture was quenched by addition water 5 mL and extracted with EA (10 mL*2). The combined organic layers were dried over [Na₂SO₄], filtered and concentrated under reduced pressure to give a residue. The residue was purified by flash silica gel chromatography (Eluent of 0-60% Ethyl acetate/Petroleum ether) to give (2S,6R)-2-(5-fluoro-1H-pyrazol-4-yl)-6-methyl-4-(p-tolylsulfonyl)morpholine (350 mg, 1.03 mmol, 62.60% yield) as yellow oil. ¹H NMR (400 MHz, CDCl₃) δ ppm 1.15-1.21 (m, 3H) 2.06-2.12 (m, 1H) 2.32 (t, J=11.00 Hz, 1H) 2.46 (s, 3H) 3.64 (dt, J=11.34, 2.03 Hz, 1H) 3.78 (dt, J=11.49, 1.96 Hz, 1H) 3.85 (ddd, J=10.21, 6.30, 2.45 Hz, 1H) 4.65 (dd, J=10.64, 2.57 Hz, 1H) 7.33-7.40 (m, 3H) 7.65 (d, J=8.19 Hz, 2H).

Step 6: A mixture of (2S,6R)-2-(5-fluoro-1H-pyrazol-4-yl)-6-methyl-4-(p-tolylsulfonyl)morpholine (1.00 eq, 100 mg, 0.295 mmol), cyclopropylboronic acid (2.00 eq, 51 mg, 0.589 mmol), DMAP (4.00 eq, 144 mg, 1.18 mmol), Pyridine (2.50 eq, 0.060 mL, 0.737 mmol) and Cu(OAc)₂ (1.00 eq, 54 mg, 0.295 mmol) in 1,4-Dioxane (10 mL) was stirred at 100° C. for 12 hours under 02 (15 psi). LCMS (5-95AB/1.5 min): RT=0.777 min, 380.3=[M+H]⁺, ESI⁺ showed starting material was consumed completely and desired mass was detected. The reaction mixture was filtered and concentrated under reduced pressure to give a residue. The residue was purified by flash silica gel chromatography (Eluent of 0-50% Ethyl acetate/Petroleum ether) (TLC:EA, UV, R_{f}=0.7) to give (2S,6R)-2-(1-cyclopropyl-5-fluoro-pyrazol-4-yl)-6-methyl-4-(p-tolylsulfonyl)morpholine (110 mg, 0.290 mmol, 98.39% yield) as yellow oil. ¹H NMR (400 MHz, CDCl₃) δ ppm 0.92-1.07 (m, 4H) 1.18 (d, J=6.25 Hz, 3H) 2.03-2.09 (m, 2H) 2.31 (t, J=11.07 Hz, 1H) 2.46 (s, 3H) 3.38-3.45 (m, 1H) 3.63 (dt, J=11.41, 2.05 Hz, 1H) 3.75 (dt, J=11.44, 2.10 Hz, 1H) 3.79-3.87 (m, 1H) 4.58 (dd, J=10.63, 2.63 Hz, 1H) 7.23-7.27 (m, 1H) 7.36 (d, J=8.00 Hz, 2H) 7.65 (d, J=8.25 Hz, 2H).

Step 7: To a solution of (2S,6R)-2-(1-cyclopropyl-5-fluoro-pyrazol-4-yl)-6-methyl-4-(p-tolylsulfonyl)morpholine (1.00 eq, 110 mg, 0.290 mmol) in Methanol (10 mL) was added Mg (powder) (15.0 eq, 104 mg, 4.35 mmol) and Mg (chips) (15.0 eq, 104 mg, 4.35 mmol) at 25° C. and then the mixture was stirred for 16 hours at 80° C. LCMS (5-95AB/1.5 min): RT=0.336 min, 226.3=[M+H]⁺, ESI⁺ showed starting material was consumed completely and one major peak with desired mass was detected. The reaction mixture was filtered and concentrated under reduced pressure to give (2S,6R)-2-(1-cyclopropyl-5-fluoro-pyrazol-4-yl)-6-methyl-morpholine (60 mg, 0.266 mmol, 91.88% yield) as white solid, which was directly used into next step.

Step 8: To a solution of 2-chloro-4-(2,4-difluorophenyl)-6,7-dimethyl-pteridine (1.00 eq, 30 mg, 0.0978 mmol) and (2S,6R)-2-(1-cyclopropyl-5-fluoro-pyrazol-4-yl)-6-methyl-morpholine (1.50 eq, 33 mg, 0.147 mmol) in DMSO (0.5 mL) was added DIEA (5.00 eq, 0.08 mL, 0.489 mmol), then the mixture was stirred at 100° C. for 20 min. LCMS (5-95AB/1.5 min): RT=1.019 min, 496.2=[M+H]⁺, ESI⁺ showed reactant was consumed completely and desired mass was detected. The reaction mixture was diluted with water 10 mL and extracted with EA (10 mL*3). The combined organic layers were dried over [Na₂SO₄], filtered and concentrated under reduced pressure to give a residue. The residue was purified by prep-HPLC (water (0.225% FA)-ACN, Phenomenex luna C18 150*25 mm*10 um) to give (2S,6R)-2-(1-cyclopropyl-5-fluoro-pyrazol-4-yl)-4-[4-(2,4-difluorophenyl)-6,7-dimethyl-pteridin-2-yl]-6-methyl-morpholine (11 mg, 0.0210 mmol, 21.51% yield) as yellow solid. LCMS: (M+H)⁺=496.2; purity=94.5% (220 nm). Retention time=0.702 min. ¹H NMR (400 MHz, CDCl₃) δ ppm 0.94-1.02 (m, 2H) 1.03-1.11 (m, 2H) 1.32 (d, J=6.25 Hz, 3H) 2.60 (s, 3H) 2.72 (s, 3H) 2.83-2.89 (m, 1H) 3.06-3.22 (m, 1H) 3.46 (tt, J=7.13, 3.75 Hz, 1H) 3.79-3.88 (m, 1H) 4.57 (dd, J=11.01, 2.50 Hz, 1H) 4.90-5.15 (m, 2H) 6.94-7.08 (m, 2H) 7.40 (d, J=2.25 Hz, 1H) 7.67-7.77 (m, 1H).

### Synthesis of Compounds I-1414 and I-1432

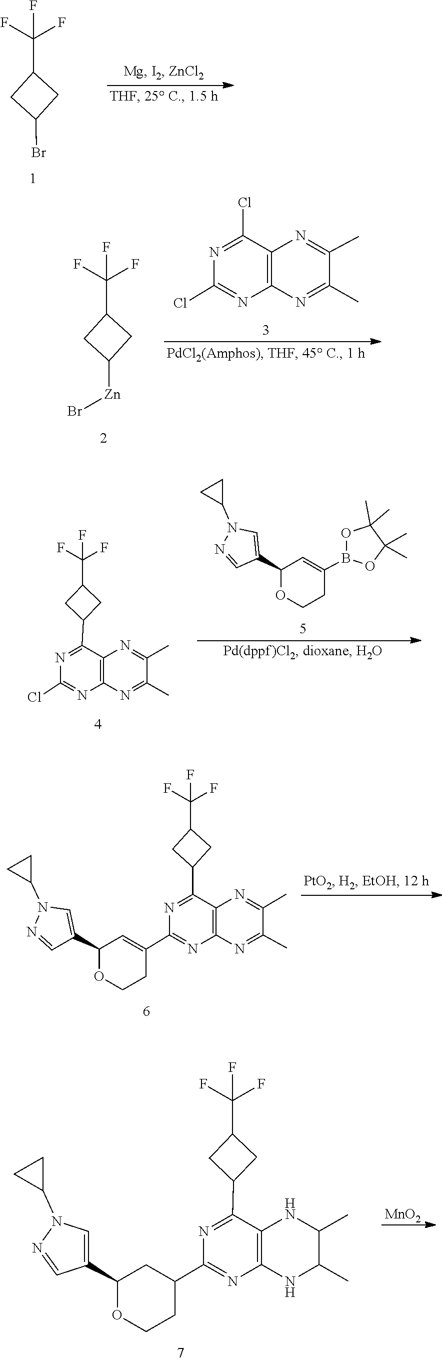

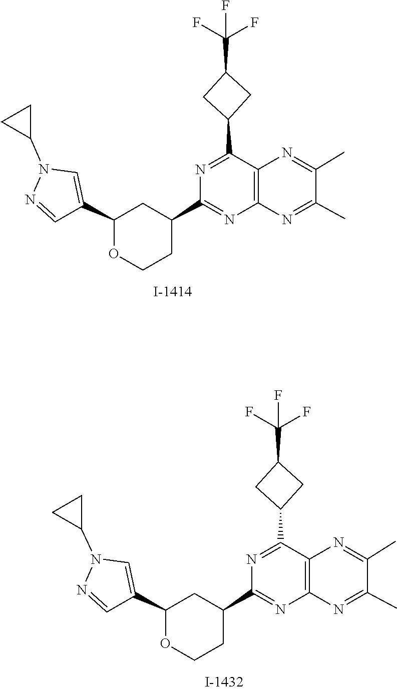 [SMILES: Cc1nc2nc([C@H]3CCO[C@@H](c4cnn(C5CC5)c4)C3)nc([C@H]3C[C@@H](C(F)(F)F)C3)c2nc1C ; Cc1nc2nc([C@H]3CCO[C@@H](c4cnn(C5CC5)c4)C3)nc([C@H]3C[C@H](C(F)(F)F)C3)c2nc1C]

Step 1: Iodine (0.050 eq, 25 mg, 0.099 mmol) in THF (0.5 mL) was added to a suspension of Mg (1.27 eq, 60 mg, 2.50 mmol) in dry THF (4 mL) under N₂ atmosphere, 1-bromo-3-(trifluoromethyl)cyclobutane (1.00 eq, 400 mg, 1.97 mmol) was added at 25° C., heated until the yellow solution turned into colorless solution, and then stirred at 25° C. for 1 h, a milky suspension was formed. ZnCl₂ (0.900 eq, 3.5 mL, 1.77 mmol) was added dropwise and the mixture was stirred for 30 minutes. A white precipitate formed. The reaction solution was used directly with syringe.

Step 2: A sealed bottle under N₂ atmosphere was charged with 2,4-dichloro-6,7-dimethyl-pteridine (1.00 eq, 100 mg, 0.437 mmol) and PdCl₂(Amphos) (0.050 eq, 15 mg, 0.0218 mmol) and THF (3 mL) and purged with N₂ three times, chloro-[3-(trifluoromethyl)cyclobutyl]zinc (4.51 eq, 441 mg, 1.97 mmol) was added dropwise to the reaction solution at 25° C., then heated to 45° C. and stirred for 1 hr. The reaction solution was changed from yellow to dark brown, the reaction was finished, the reaction solution was poured into H₂O (10 mL), extracted with EtOAc (10 mL*2), dried over Na₂SO₄ and evaporated under reduced pressure to give 2-chloro-6,7-dimethyl-4-[3-(trifluoromethyl)cyclobutyl]pteridine (60 mg, 0.189 mmol, 43.40% yield) as yellow solid. [M+H]⁺=317.0; purity=92.4% (220 nm). Retention time=0.922 min. ¹H NMR (400 MHz, CDCl₃) δ=5.00-4.83 (m, 1H), 3.28-3.11 (m, 1H), 2.85-2.74 (m, 10H).

Step 3: To a solution of 2-chloro-6,7-dimethyl-4-[3-(trifluoromethyl)cyclobutyl]pteridine (1.00 eq, 60 mg, 0.189 mmol),1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 66 mg, 0.208 mmol) and K₂CO₃ (2.00 eq, 32 mg, 0.379 mmol) in 1,4-Dioxane (2 mL) and water (0.4 mL), [1,1′-Bis(diphenylphosphino)ferrocene]dichloropalladium(II) (0.0800 eq, 11 mg, 0.0152 mmol) was added and purged with N₂ for 3 times, the reaction solution was stirred at 100° C. for 2 hrs, LCMS showed the reactant was consumed and 61% of desired mass was detected. the reaction solution was poured into H₂O (5 mL), extracted with EtOAc (5 mL*2), dried over Na₂SO₄ and evaporated under reduced pressure to give the residue, which was then purified with Prep-TLC (pure EA, Rf=0.4) to give 2-[(6R)-6-(1-cyclopropylpyrazol-4-yl)-3,6-dihydro-2H-pyran-4-yl]-6,7-dimethyl-4-[3-(trifluoromethyl)cyclobutyl]pteridine (56 mg, 0.119 mmol, 62.83% yield) as yellow solid. [M+H]⁺=471.2; purity=94.5% (220 nm). Retention time=0.953 min.

Step 4: To a solution of 2-[(6R)-6-(1-cyclopropylpyrazol-4-yl)-3,6-dihydro-2H-pyran-4-yl]-6,7-dimethyl-4-[3-(trifluoromethyl)cyclobutyl]pteridine (1.00 eq, 56 mg, 0.119 mmol) in ethanol (5 mL) was added PtO₂ (1.00 eq, 27 mg, 0.119 mmol) under N₂ atmosphere. The mixture was purged with H₂ for 3 times, then the mixture was stirred under H₂ atmosphere (15 psi) at 25° C. for 12 h. LCMS showed the reactant was consumed and 95% of desired mass was detected, the reaction mixture was filtered through celite and evaporated under reduced pressure to give 2-[(2R)-2-(1-cyclopropylpyrazol-4-yl)tetrahydropyran-4-yl]-6,7-dimethyl-4-[3-(trifluoromethyl)cyclobutyl]-5,6,7,8-tetrahydropteridine (55 mg, 0.115 mmol, 96.97% yield) as yellow oil. [M+H]⁺=477.1; purity=95% (220 nm). Retention time=0.725 min.

Step 5: To a solution of 2-[(2R)-2-(1-cyclopropylpyrazol-4-yl)tetrahydropyran-4-yl]-6,7-dimethyl-4-[3-(trifluoromethyl)cyclobutyl]-5,6,7,8-tetrahydropteridine (1.00 eq, 55 mg, 0.115 mmol) in DCM (4 mL) was added MnO₂ (15.0 eq, 151 mg, 1.73 mmol) and stirred at 25° C. for 12 h. LCMS showed the reactant was consumed and 90% of desired mass was detected, the reaction mixture was filtered through celite, evaporated under reduced pressure to give the residue, which was then purified with Prep-HPLC (FA) and lyophilized to give 2-[(2R)-2-(1-cyclopropylpyrazol-4-yl)tetrahydropyran-4-yl]-6,7-dimethyl-4-[3-(trifluoromethyl)cyclobutyl]pteridine (5.7 mg, 0.0121 mmol, 10.45% yield) as off-white solid and 2-[(2R)-2-(1-cyclopropylpyrazol-4-yl)tetrahydropyran-4-yl]-6,7-dimethyl-4-[3-(trifluoromethyl)cyclobutyl]pteridine (22 mg, 0.0463 mmol, 40.09% yield) as off-white solid. [M+H]⁺=473.2; purity=99.6% (220 nm). Retention time=2.387 min. ¹H NMR (400 MHz, CDCl₃) δ=7.52 (s, 2H), 4.68 (t, J=9.0 Hz, 1H), 4.61-4.52 (m, 1H), 4.31-4.24 (m, 1H), 3.89-3.76 (m, 1H), 3.62-3.52 (m, 1H), 3.52-3.43 (m, 1H), 3.23-3.06 (m, 1H), 2.84-2.74 (m, 8H), 2.70-2.60 (m, 2H), 2.48-2.41 (m, 1H), 2.26-2.10 (m, 3H), 1.15-1.08 (m, 2H), 1.04-0.96 (m, 2H). [M+H]⁺=473.2; purity=97.9% (220 nm). Retention time=2.443 min. ¹H NMR (400 MHz, CDCl₃) δ=7.52 (d, J=1.3 Hz, 2H), 4.90 (quin, J=8.1 Hz, 1H), 4.58 (dd, J=1.9, 11.4 Hz, 1H), 4.32-4.24 (m, 1H), 3.87-3.77 (m, 1H), 3.58-3.42 (m, 2H), 3.26-3.08 (m, 1H), 2.86-2.74 (m, 9H), 2.44 (br d, J=13.3 Hz, 1H), 2.21-2.11 (m, 3H), 1.12-1.07 (m, 2H), 1.02-0.95 (m, 2H).

### Synthesis of Compound I-1433

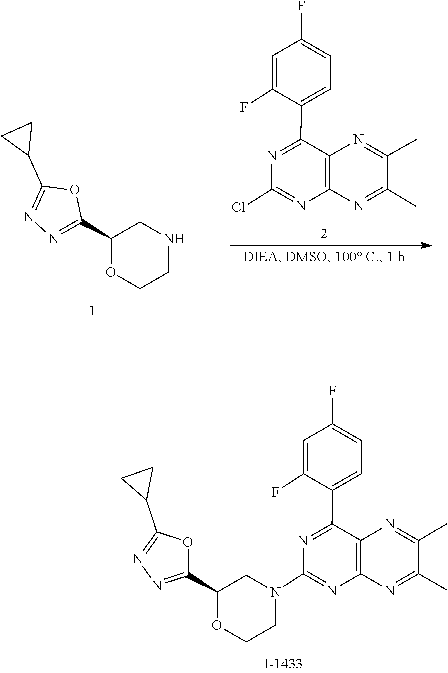 [SMILES: C1CO[C@@H](c2nnc(C3CC3)o2)CN1 ; Cc1nc2nc(Cl)nc(-c3ccc(F)cc3F)c2nc1C ; Cc1nc2nc(N3CCO[C@@H](c4nnc(C5CC5)o4)C3)nc(-c3ccc(F)cc3F)c2nc1C]

To a solution of (2R)-2-(5-cyclopropyl-1, 3, 4-oxadiazol-2-yl) morpholine (1.00 eq, 100 mg, 0.512 mmol) and 2-chloro-4-(2, 4-difluorophenyl)-6, 7-dimethyl-pteridine (0.500 eq, 79 mg, 0.256 mmol) in DMSO (1 mL) was added DIEA (4.00 eq, 0.34 mL, 2.05 mmol). The mixture was stirred at 100° C. for 1 hour. LCMS showed the starting material was consumed completely and a new peak with desired mass (12%, MS: 466.1 [M+H]⁺, ESI pos). The reaction was filtered and the filtrate was purified by prep-HPLC (flow: 25 mL/min; gradient: from 38-68% water (0.1% FA)-ACN over 7 min; column: Unisil 3-100 C18 Ultra 150*25 mm*10 um) and lyophilized to afford (2R)-2-(5-cyclopropyl-1,3,4-oxadiazol-2-yl)-4-[4-(2,4-difluorophenyl)-6,7-dimethyl-pteridin-2-yl]morpholine (2.3 mg, 0.00434 mmol, 0.8500% yield) as brown solid. Rt: 0.926 min, m/z: 466.1 [M+H]⁺. 96.54% purity at 220 nm. 1H NMR (400 MHz, CDCl3) δ ppm 1.11-1.21 (m, 4H) 2.13-2.21 (m, 1H) 2.61 (s, 3H) 2.73 (s, 3H) 3.50 (ddd, J=13.76, 10.88, 3.38 Hz, 1H) 3.64-3.75 (m, 1H) 3.85 (td, J=11.07, 2.38 Hz, 1H) 4.17 (dt, J=11.44, 2.78 Hz, 1H) 4.85 (dd, J=10.13, 2.88 Hz, 2H) 5.17 (br d, J=12.51 Hz, 1H) 6.94-7.08 (m, 2H) 7.72 (td, J=8.13, 6.75 Hz, 1H).

### Synthesis of Compound I-1436

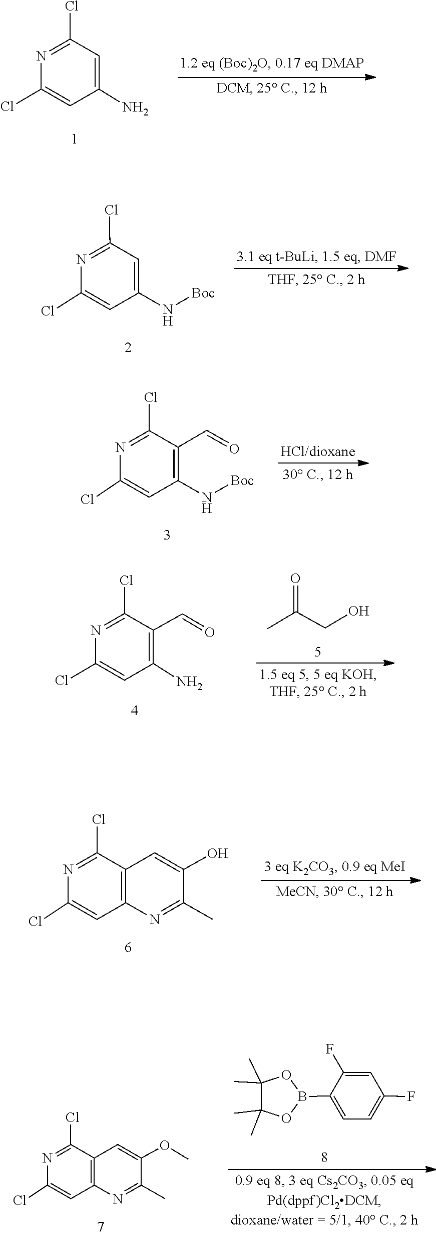

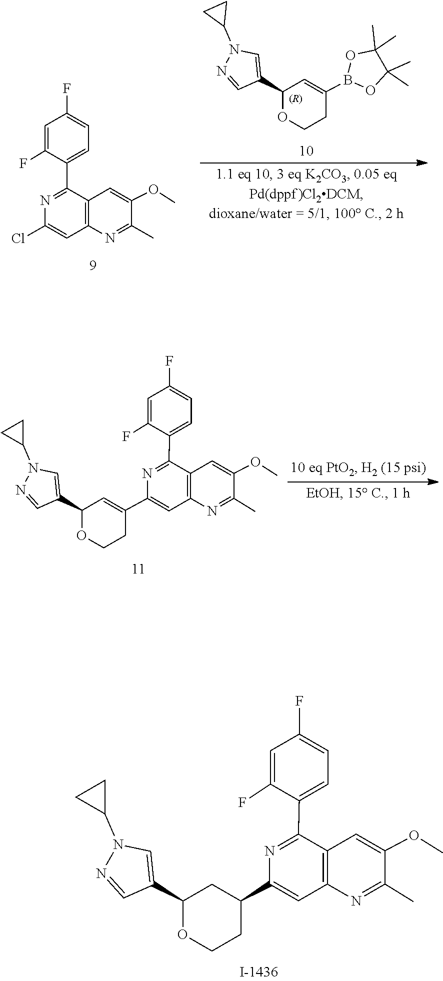

Step 1: To a solution of 2, 6-dichloropyridin-4-amine (1.00 eq, 5000 mg, 30.7 mmol) in DCM (150 mL) was added (Boc)₂O (1.00 eq, 7.1 mL, 30.7 mmol) and DMAP (0.170 eq, 636 mg, 5.21 mmol) at 0° C., then stirred at 30° C. for 16 hours. TLC (PE/EA=3/1, starting material Rf=0.1; PE/EA=3/1, new spot Rf=0.5) showed a new spot was detected. The reaction mixture was poured into saturated NH₄Cl aqueous solution (100 mL) and then extracted with ethyl acetate (100 mL*3). The combined organic layers were dried over Na₂SO₄, filtered and concentrated under reduced pressure to give a residue. The residue was purified by flash chromatography on silica gel eluting with PE/EtOAc (100/1 to 3/1) (TLC, PE:EtOAc=5:1, Rf=0.60) to afford the product tert-butyl N-(2,6-dichloro-4-pyridyl) carbamate (7000 mg, 21.3 mmol, 69.38% yield), which LCMS (M-56+H)⁺=207.1; purity=80% (254 nm). Retention time=0.930 min.

Step 2: A solution of tert-butyl N-(2,6-dichloro-4-pyridyl)carbamate (1.00 eq, 500 mg, 1.90 mmol) in THF (8 mL) was purged with N₂ for 3 times at −78° C., t-butyl lithium/pentane (3.10 eq, 4.5 mL, 5.89 mmol) was added slowly with temperature kept at 0° C. and stirred for 0.5 hour, then DMF (1.50 eq, 208 mg, 2.85 mmol) was added and warmed to 25° C. and stirred for 2 hours. TLC (PE/EA=5/1, starting material Rf=0.1; new spot Rf=0.5, UV) showed a new spot was detected. The reaction mixture was poured into saturated NH₄Cl aqueous solution (100 mL) and then extracted with ethyl acetate (10 mL* 3). The combined organic layers were dried over Na₂SO₄, filtered and concentrated under reduced pressure to give a residue. The residue was purified by flash chromatography on silica gel eluting with PE/EtOAc (100/1 to 3/1) (TLC, PE:EtOAc=5:1, Rf=0.50) to afford the product tert-butyl N-(2,6-dichloro-4-pyridyl)carbamate (7000 mg, 21.3 mmol, 69.38% yield), which was H NMR. 1H NMR (400 MHz, CDCl₃) δ=11.11-10.98 (m, 1H), 10.47-10.45 (m, 1H), 8.48 (s, 1H), 1.55 (s, 9H).

Step 3: A solution of tert-butyl N-(2,6-dichloro-3-formyl-4-pyridyl)carbamate (1.00 eq, 250 mg, 0.859 mmol) in HCl/dioxane (4 M, 11.6 eq, 2.5 mL, 10.0 mmol) was stirred at 30° C. for 1 hour. LCMS (5-95AB/1.5 min): RT=0.564 min, 191.0=[M+H]⁺, ESI⁺ showed 91% of desired product. The reaction mixture was poured into saturated NaHCO₃ aqueous solution (20 mL) and then extracted with ethyl acetate (20 mL*3). The combined organic layers were dried over Na₂SO₄, filtered and concentrated under reduced pressure to give crude 4-amino-2,6-dichloro-pyridine-3-carbaldehyde (200 mg, 0.984 mmol, 114.62% yield), which was LCMS: (M+H)⁺=191.0; purity=94% (220 nm). Retention time=0.564 min.

Step 4: To a solution of 4-amino-2,6-dichloro-pyridine-3-carbaldehyde (1.00 eq, 200 mg, 1.05 mmol) in THF (5 mL) was added 1-hydroxypropan-2-one (1.50 eq, 116 mg, 1.57 mmol) and KOH (5.00 eq, 293 mg, 5.24 mmol), then the mixture was stirred at 25° C. for 12 hours. LCMS (5-95AB/1.5 min): RT=0.812 min, 229.0=[M]⁺, ESI⁺ showed 90% of desired product. The reaction mixture was poured into saturated NH₄Cl aqueous solution (20 mL) and then extracted with ethyl acetate (20 mL*3). The combined organic layers were dried over Na₂SO₄, filtered and concentrated under reduced pressure to give a residue. (M+H)⁺=229.0; purity=90% (220 nm). Retention time=0.812 min.

Step 5: To a solution suspension of 5,7-dichloro-2-methyl-1,6-naphthyridin-3-ol (1.00 eq, 150 mg, 0.655 mmol) and K₂CO₃ (3.00 eq, 272 mg, 1.96 mmol) in MeCN (3 mL) was added and Mel (0.900 eq, 0.037 mL, 0.589 mmol) stirred at 30° C. for 1 hour. LCMS (5-95AB/1.5 min): RT=0.899 min, 243.0=[M]⁺, ESI⁺ showed 28% of desired product and RT=0.816 min, 229.0=[M]⁺, ESI⁺ showed 70% of starting material. After 12 h, LCMS (5-95AB/1.5 min): RT=0.896 min, 243.0=[M]⁺, ESI⁺ showed 90% of desired product. The reaction mixture was poured into saturated NH₄Cl aqueous solution (5 mL) and then extracted with ethyl acetate (20 mL*3). The combined organic layers were dried over Na₂SO₄, filtered and concentrated under reduced pressure to give crude 5,7-dichloro-3-methoxy-2-methyl-1,6-naphthyridine (180 mg, 0.740 mmol, 113.07% yield), which was LCMS: (M+H)⁺=243.0; purity=97% (220 nm). Retention time=0.889 min.

Step 6: A solution of 5,7-dichloro-3-methoxy-2-methyl-1,6-naphthyridine (1.00 eq, 180 mg, 0.740 mmol) and 2-(2,4-difluorophenyl)-4,4,5,5-tetramethyl-1,3,2-dioxaborolane (0.900 eq, 160 mg, 0.666 mmol) and Cs₂CO₃ (3.00 eq, 722 mg, 2.22 mmol) and Pd(dppf)Cl₂·CH₂Cl₂ (0.0500 eq, 30 mg, 0.0370 mmol) in 1,4-Dioxane (5 mL) and Water (1 mL) was purged with N₂ for 3 times and stirred at 40° C. for 2 hours. LCMS (5-95AB/1.5 min): RT=0.951 min, 321.2=[M+H]⁺, ESI⁺ showed 40% of desired product. The reaction mixture was extracted with ethyl acetate (20 mL*3). The combined organic layers were dried over Na₂SO₄, filtered and concentrated under reduced pressure to give a residue. The residue was purified by prep-TLC (PE:EtOAc=1:1, Rf=0.55) to afford 7-chloro-5-(2,4-difluorophenyl)-3-methoxy-2-methyl-1,6-naphthyridine (120 mg, 0.299 mmol, 40.42% yield) as yellow solid. (M+H)⁺=321.2; purity=40% (220 nm). Retention time=0.951 min. 1H NMR (400 MHz, CDCl₃) δ=7.91 (s, 1H), 7.67-7.59 (m, 2H), 7.53-7.37 (m, 3H), 7.13-7.01 (m, 3H), 3.87-3.84 (m, 3H), 2.70 (s, 3H).

Step 7: A solution of 7-chloro-5-(2,4-difluorophenyl)-3-methoxy-2-methyl-1,6-naphthyridine (1.00 eq, 100 mg, 0.312 mmol) and 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 108 mg, 0.343 mmol) and K₂CO₃ (3.00 eq, 129 mg, 0.935 mmol) and Pd(dppf)Cl₂CH₂Cl₂ (0.0900 eq, 23 mg, 0.0281 mmol) in 1,4-Dioxane (0.5 mL) and water (0.1 mL) was purged with N₂ for 3 times stirred at 100° C. for 2 hours. LCMS (5-95AB/1.5 min): RT=0.939 min, 475.2=[M+H]⁺, ESI⁺ showed 59% of desired product. The reaction mixture was poured into saturated NH₄Cl aqueous solution (20 mL) and then extracted with ethyl acetate (20 mL*3). The combined organic layers were dried over Na₂SO₄, filtered and concentrated under reduced pressure to give a residue. The residue was purified by prep-TLC (PE:EtOAc=0:1, Rf=0.55) to afford 7-[(6R)-6-(1-cyclopropylpyrazol-4-yl)-3,6-dihydro-2H-pyran-4-yl]-5-(2,4-difluorophenyl)-3-methoxy-2-methyl-1,6-naphthyridine (40 mg, 0.0531 mmol, 17.03% yield) as a yellow oil, which LCMS (5-95AB/1.5 min): RT=0.960 min, 475.2=[M+H]⁺, ESI⁺ showed 63% of desired mass. (M+H)⁺=475.2; purity=63% (220 nm). Retention time=0.960 min.

Step 8: A solution of 7-[(6R)-6-(1-cyclopropylpyrazol-4-yl)-3,6-dihydro-2H-pyran-4-yl]-5-(2,4-difluorophenyl)-3-methoxy-2-methyl-1,6-naphthyridine (1.00 eq, 40 mg, 0.0843 mmol) and PtO₂ (0.500 eq, 9.6 mg, 0.0421 mmol) in Ethanol (2 mL) was purged with H₂ for 3 times and stirred at 30° C. for 2 hours. LCMS (5-95AB/1.5 min): RT=0.892 min, 477.2=[M+H]⁺, ESI⁺ showed 38% of desired product. The reaction mixture was filtered by celite and extracted with EtOAc (50 mL) twice. The combined organic layers were washed with brine (50 mL), dried over Na₂SO₄ and concentrated in vacuo to give the residue. The residue was purified by prep-HPLC (Column: Unisil 3-100 C18 Ultra 150*50 mm*3 um), Condition: water (FA)-ACN, Gradient Time (min): 7). The purified solution was lyophilized to afford the product 7-[(2R,4S)-2-(1-cyclopropylpyrazol-4-yl)tetrahydropyran-4-yl]-5-(2,4-difluorophenyl)-3-methoxy-2-methyl-1,6-naphthyridine (2.4 mg, 0.00488 mmol, 5.79% yield) as white solid (M+H)⁺=477.2; purity=100% (220 nm). Retention time=0.923 min. 1H NMR (400 MHz, CDCl3) δ=7.73 (s, 1H), 7.60 (dt, J=6.5, 8.3 Hz, 1H), 7.48 (d, J=3.7 Hz, 2H), 7.13-7.07 (m, 2H), 7.01 (dt, J=2.4, 9.4 Hz, 1H), 4.56 (dd, J=1.7, 11.2 Hz, 1H), 4.29-4.23 (m, 1H), 3.85 (s, 3H), 3.81-3.77 (m, 1H), 3.56 (td, J=3.6, 7.3 Hz, 1H), 3.31 (tt, J=3.6, 11.9 Hz, 1H), 2.69 (s, 3H), 2.42-2.35 (m, 1H), 2.10-1.94 (m, 3H), 1.13-1.07 (m, 2H), 1.01-0.96 (m, 2H).

### Synthesis of Compound 1-1441

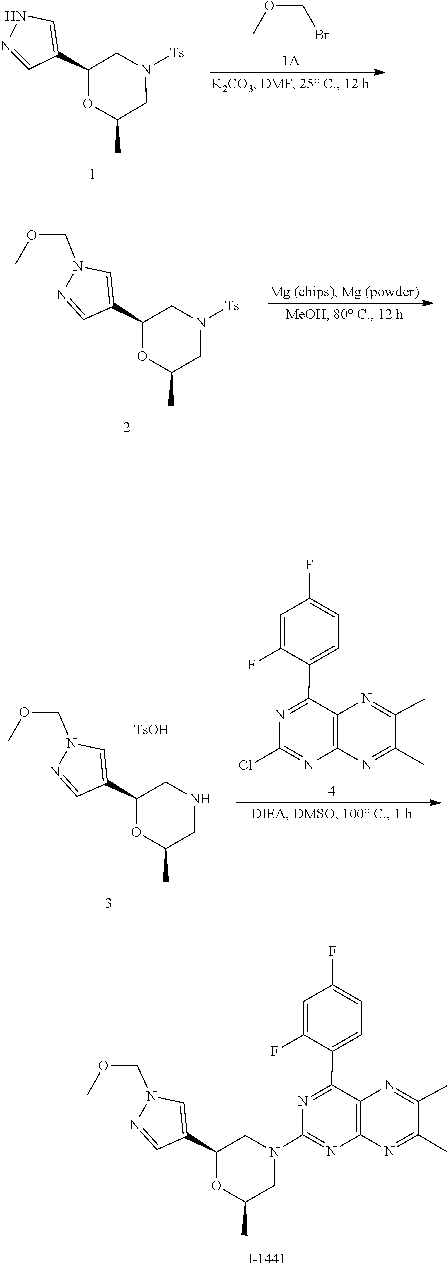

Step 1: A solution of (2R,6S)-2-methyl-4-(p-tolylsulfonyl)-6-(1H-pyrazol-4-yl)morpholine (1.00 eq, 200 mg, 0.622 mmol), bromo(methoxy)methane (1.70 eq, 132 mg, 1.06 mmol) and K₂CO₃ (2.00 eq, 172 mg, 1.24 mmol) in DMF (2 mL) was stirred at 25° C. for 1 h. LCMS showed the reactant was consumed and the desired mass was detected, the reaction solution was poured into H₂O (10 mL), extracted with EtOAc (10 mL*2) to give the organic layer, which was then washed with saturated NaCl solution, dried over Na₂SO₄ and evaporated to give (2R,6S)-2-[1-(methoxymethyl)pyrazol-4-yl]-6-methyl-4-(p-tolylsulfonyl)morpholine (150 mg, 0.410 mmol, 65.96% yield) as yellow oil. [M+H]⁺=366.1; purity=97.4% (220 nm). Retention time=0.851 min.

Step 2: To a solution of (2R,6S)-2-[1-(methoxymethyl)pyrazol-4-yl]-6-methyl-4-(p-tolylsulfonyl)morpholine (1.00 eq, 150 mg, 0.410 mmol) in methanol (25 mL) was added Mg (powder) (12.5 eq, 124 mg, 5.15 mmol) and Mg (chips) (12.5 eq, 124 mg, 5.15 mmol) at 25° C. and then purged with N₂ for 3 times and stirred for 12 h at 80° C. LCMS showed 72% of desired mass was detected and 16% of SM remained, then another Mg (chips) (12.5 eq, 124 mg, 5.15 mmol) and Mg (powder) (12.5 eq, 124 mg, 5.15 mmol) was added and stirred at 80° C. for another 12 h. LCMS showed 78% of desired mass was detected. the reaction mixture was filtered through celite with MeOH and EtOAc, then dried in vacuo to give (2S,6R)-2-[1-(methoxymethyl)pyrazol-4-yl]-6-methyl-morpholine;4-methylbenzenesulfonic acid (162 mg, 0.422 mmol, 102.93% yield) as yellow solid. [M+H]⁺=212.2; purity=78% (220 nm). Retention time=0.248 min.

Step 3: To a solution of (2S,6R)-2-[1-(methoxymethyl)pyrazol-4-yl]-6-methyl-morpholine;4-methylbenzenesulfonic acid (1.00 eq, 80 mg, 0.209 mmol) and 2-chloro-4-(2,4-difluorophenyl)-6,7-dimethyl-pteridine (1.00 eq, 64 mg, 0.209 mmol) in DMSO (2 mL) was added DIEA (5.00 eq, 135 mg, 1.04 mmol), then the mixture was stirred at 100° C. for 1 h. LCMS showed the reactant was consumed and 41% of desired mass was detected. the reaction solution was poured into H₂O (10 mL), extracted with EtOAc (10 mL*2), dried over Na₂SO₄ and evaporated under reduced pressure to give the residue, which was then purified with prep-HPLC (FA) and lyophilized to give (2S,6R)-4-[4-(2,4-difluorophenyl)-6,7-dimethyl-pteridin-2-yl]-2-[1-(methoxymethyl)pyrazol-4-yl]-6-methyl-morpholine (7.2 mg, 0.0148 mmol, 7.10% yield) as yellow solid. [M+H]⁺=482.1; purity=98.7% (220 nm). Retention time=0.956 min. 1H NMR (400 MHz, CDCl₃) δ=7.66 (d, J=3.1 Hz, 3H), 7.07-6.94 (m, 2H), 5.38 (s, 2H), 5.21-4.92 (m, 2H), 4.66 (dd, J=2.8, 11.0 Hz, 1H), 3.85 (ddd, J=2.5, 6.3, 10.4 Hz, 1H), 3.35 (s, 3H), 3.09 (dd, J=10.9, 13.3 Hz, 1H), 2.90-2.83 (m, 1H), 2.72 (s, 3H), 2.60 (s, 3H), 1.34 (d, J=6.1 Hz, 3H).

### Synthesis of Compound 1-1446

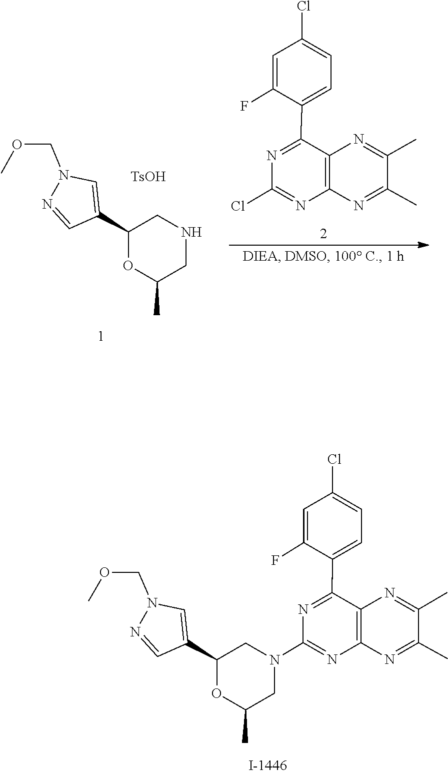

To a solution of (2S,6R)-2-[1-(methoxymethyl)pyrazol-4-yl]-6-methyl-morpholine;4-methylbenzenesulfonic acid (1.00 eq, 80 mg, 0.209 mmol) and 2-chloro-4-(4-chloro-2-fluoro-phenyl)-6,7-dimethyl-pteridine (1.00 eq, 67 mg, 0.209 mmol) in DMSO (2 mL) was added DIEA (5.00 eq, 135 mg, 1.04 mmol), then the mixture was stirred at 100° C. for 1 h. LCMS showed the reactant was consumed and 45% of desired mass was detected. the reaction solution was poured into H₂O (10 mL), extracted with EtOAc (10*2 mL), dried over Na₂SO₄ and evaporated under reduced pressure to give the residue, which was then purified with prep-HPLC (FA) and lyophilized to give (2S,6R)-4-[4-(4-chloro-2-fluoro-phenyl)-6,7-dimethyl-pteridin-2-yl]-2-[1-(methoxymethyl)pyrazol-4-yl]-6-methyl-morpholine (8.6 mg, 0.0173 mmol, 8.30% yield) as yellow solid. [M+H]⁺=498.2; purity=100% (220 nm). Retention time=0.978 min. 1H NMR (400 MHz, CDCl₃) δ=7.69-7.62 (m, 3H), 7.32 (br d, J=8.1 Hz, 2H), 5.38 (s, 2H), 5.20-4.89 (m, 2H), 4.66 (dd, J=1.9, 10.6 Hz, 1H), 3.85 (ddd, J=2.5, 6.4, 10.6 Hz, 1H), 3.35 (s, 3H), 3.09 (dd, J=10.9, 13.3 Hz, 1H), 2.86 (dd, J=10.7, 13.3 Hz, 1H), 2.72 (s, 3H), 2.60 (s, 3H), 1.34 (d, J=6.3 Hz, 3H).

### Synthesis of I-1451 (Same general method for I-1495 and I-1500)

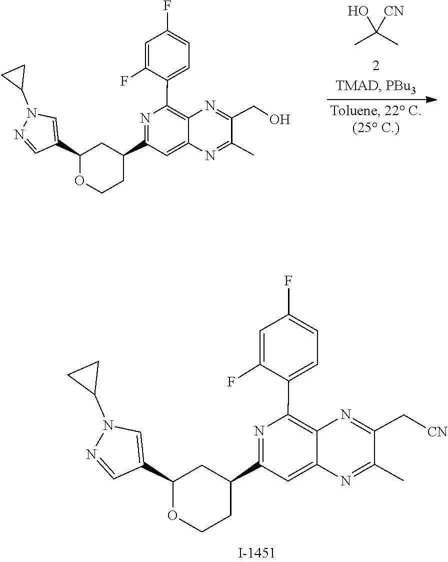

A flame-dried round-bottomed flask equipped with a Teflon-coated magnetic stirring bar was charged with (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methanol (1.0 equiv, 48 mg, 0.10 mmol). The flask was sealed and purged under Ar (g). Anhydrous toluene (3.0 mL) was added, and the resulting solution was stirred at 22° C. Acetone cyanohydrin 2 (2.00 equiv, 0.018 mL, 0.201 mmol), (3E)-3-(dimethylcarbamoylimino)-1,1-dimethyl-urea (1.50 equiv, 26 mg, 0.151 mmol) and tri-n-butylphosphine (1.50 equiv, 0.037 mL, 0.151 mmol) were added sequentially. The formation of an orange precipitate was noted. The reaction mixture was stirred at 22° C. for 24 h. The reaction mixture was diluted with EtOAc and washed sequentially with sat. NH₄Cl (aq), sat. NaHCO₃ (aq) and brine. The organic layer was dried over Na₂SO₄, filtered and evaporated to dryness under reduced pressure. The crude residual mixture was purified by silica gel flash column chromatography (Teledyne RediSep® GOLD column, 24 g SiO₂) using an elution gradient of 0% to 10% MeOH in DCM to afford 54 mg of the crude product as a yellow-orange syrup. The product was further purified by preparative HPLC (Gemini® 5 um NX-C18 110 Å, 100×30 mm column) using an elution gradient of MeOH in 10 mM aqueous ammonium formate pH 3.8 (50-70%) to afford 2-(7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)acetonitrile (12 mg, 0.025 mmol, 25% yield) as a white solid. LC-MS(ESI⁺): Tr=1.44 min; [M+H]⁺ 487.3 (obs). ¹H NMR (DMSO-d₆, 400 MHz): ^{6H} 7.89 (1H, s), 7.71-7.77 (2H, m), 7.36-7.40 (2H, m), 7.26 (1H, td, J=8.4, 2.6 Hz), 4.56 (2H, s), 4.49 (1H, d, J=11.1 Hz), 4.09 (1H, dd, J=11.2, 4.0 Hz), 3.60-3.71 (2H, m), 2.71 (3H, s), 2.22 (1H, d, J=13.0 Hz), 1.86-1.95 (4H, m), 1.23 (1H, s), 0.95-1.00 (2H, m), 0.88-0.93 (2H, m).

### Synthesis of 1-1456

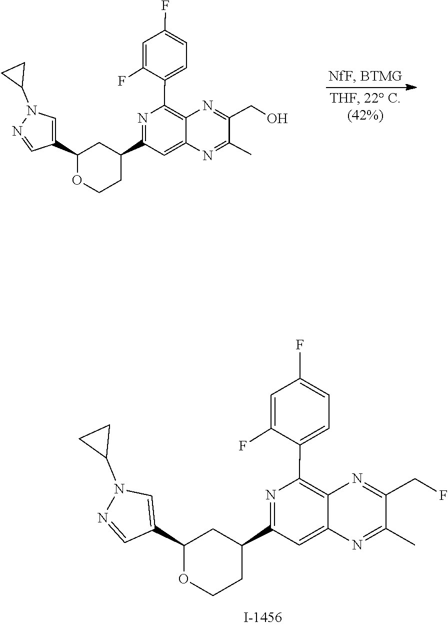 [SMILES: Cc1nc2cc([C@H]3CCO[C@@H](c4cnn(C5CC5)c4)C3)nc(-c3ccc(F)cc3F)c2nc1CO ; Cc1nc2cc([C@H]3CCO[C@@H](c4cnn(C5CC5)c4)C3)nc(-c3ccc(F)cc3F)c2nc1CF]

A flame-dried round-bottomed flask equipped with a Teflon-coated magnetic stirring bar was charged with (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methanol (1.0 equiv, 51 mg, 0.10 mmol) and anhydrous THF (1.5 mL). The flask was sealed, purged under Ar (g) and cooled in an ice bath at 0° C. with stirring. Perfluorobutanesulfonyl fluoride (1.20 equiv, 0.022 mL, 0.121 mmol) was added, followed by 2-tert-butyl-1,1,3,3-tetramethylguanidine (1.10 equiv, 0.023 mL, 0.111 mmol). The reaction mixture was stirred at 22° C. for 22 h, after which additional perfluorobutanesulfonyl fluoride (1.20 equiv, 0.022 mL, 0.121 mmol) and 2-tert-butyl-1,1,3,3-tetramethylguanidine (1.10 equiv, 0.023 mL, 0.111 mmol) were added. The reaction mixture was stirred at 22° C. for an additional 24 h, after which additional perfluorobutanesulfonyl fluoride (1.20 equiv, 0.022 mL, 0.121 mmol) and 2-tert-butyl-1,1,3,3-tetramethylguanidine (1.10 equiv, 0.023 mL, 0.111 mmol) were added. After 72 h reaction time, the flask was cooled to 0° C. in an ice bath and the reaction mixture was quenched with sat. NaHCO₃ (aq). The reaction mixture was warmed to 22° C., diluted with EtOAc and washed successively with sat. NH₄Cl (aq), sat. NaHCO₃ (aq) and brine. The organic layer was dried over Na₂SO₄, filtered and evaporated to dryness under reduced pressure. The crude residual mixture was purified by silica gel flash column chromatography (Teledyne RediSep® GOLD column, 24 g SiO₂) using an elution gradient of 0% to 10% MeOH in DCM to afford 40 mg of the crude product as a white foam. The product was further purified by preparative HPLC (Gemini® 5 um NX-C18 110 Å, 100×30 mm column) using an elution gradient of MeOH in 10 mM aqueous ammonium formate pH 3.8 (55-75%) to afford 7-((2R,4S)-2-(1-cyclopropyl-1H-pyrazol-4-yl)tetrahydro-2H-pyran-4-yl)-5-(2,4-difluorophenyl)-3-(fluoromethyl)-2-methylpyrido[3,4-b]pyrazine (20 mg, 0.04 mmol, 42% yield) as a white solid. LC-MS(ESI+): Tr=1.60 min; [M+H]+ 480.3 (obs). ¹H NMR (DMSO-d₆, 400 MHz): ^{6H} 7.91 (1H, s), 7.69-7.74 (2H, m), 7.39-7.44 (2H, m), 7.27 (1H, t, J=8.6 Hz), 5.70 (2H, d, J=46.5 Hz), 4.49 (1H, d, J=11.1 Hz), 4.09 (1H, d, J=11.2 Hz), 3.62-3.72 (2H, m), 3.33-3.35 (2H, m), 2.79 (3H, s), 2.23 (1H, d, J=13.1 Hz), 1.86-1.98 (3H, m), 0.98-0.99 (2H, m), 0.90-0.92 (2H, m).

### Synthesis of I-1461

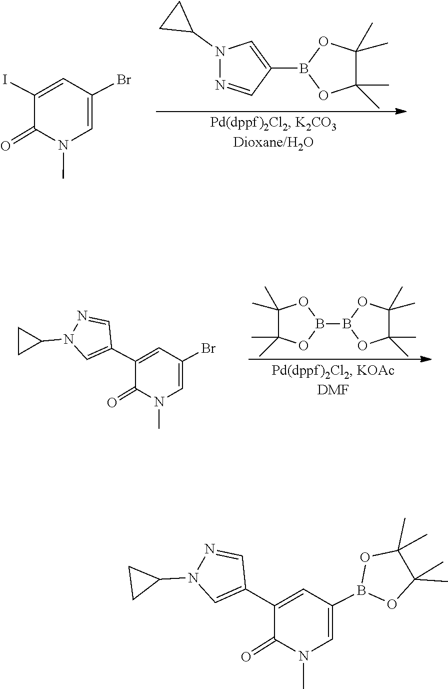

Step 1: To a solution of 5-bromo-3-iodo-1-methyl-pyridin-2-one (1.0 eq, 3.00 g, 9.6 mmol) and 1-cyclopropyl-4-(4,4,5,5-tetramethyl-1,3,2-dioxaborolan-2-yl)pyrazole (1.1 eq, 2.46 g, 10.5 mmol) in 1,4-dioxane (60 mL) was added water (15 ml), K₂CO₃ (3.00 eq, 3.96 g, 28.7 mmol) and Pd(dppf)₂Cl₂ (0.05 eq, 390 mg, 0.5 mmol). The mixture was stirred at 80° C. until completion was indicated by TLC. The reaction mixture was cooled down then water and EtOAc were added, and the mixture was extracted with EtOAc. The combined organic layers were washed with brine, dried over MgSO₄, filtered, and concentrated in vacuo. The crude material was purified by silica gel chromatography using a 120 g pre-packed column eluting with 60 to 90% EtOAc in Hexanes to provide 5-bromo-3-(1-cyclopropyl-1H-pyrazol-4-yl)-1-methylpyridin-2(1H)-one (2.36 g, 8.0 mmol, 84% yield) as a light-yellow solid. ¹H NMR (400 MHz, Chloroform-d)^{6H} 8.36 (1H, s), 7.78 (1H, d, J=0.7 Hz), 7.57 (1H, d, J=2.6 Hz), 3.59-3.54 (1H, m), 3.53 (3H, s), 1.11-1.07 (2H, m), 0.99-0.94 (2H, m).

Step 2: To a solution of 5-bromo-3-(1-cyclopropylpyrazol-4-yl)-1-methyl-pyridin-2-one (1.0 eq, 1.50 g, 5.1 mmol) in DMF (20 mL) was added bis(pinacolato)diboron (1.1 eq, 1.42 g, 5.6 mmol), K₂CO₃ (3.0 eq, 1.50 g, 15.3 mmol) and Pd(dppf)Cl₂ (0.05 eq, 208 mg, 0.26 mmol). The mixture was stirred at 80° C. until completion was indicated by TLC. The reaction mixture was cooled down to room temperature and water and EtOAc were added. The organic phase was collected, and the aqueous layer was extracted with EtOAc. The combined organic layers were washed with water, brine, dried over MgSO₄, filtered, and concentrated in vacuo. The crude material was purified by silica gel chromatography using a 120 g pre-packed column eluting with 60 to 90% EtOAc in Hexanes to provide 3-(1-cyclopropyl-1H-pyrazol-4-yl)-1-methyl-5-(4,4,5,5-tetramethyl-1,3,2-dioxaborolan-2-yl)pyridin-2(1H)-one (1.5 g, 4.37 mmol, 86% yield) as a light-yellow solid. ¹H NMR (400 MHz, Chloroform-d) δ_{H} 8.20 (1H, s), 7.75 (1H, d, J=0.7 Hz), 7.70 (1H, d, J=1.9 Hz), 7.54 (1H, d, J=1.9 Hz), 3.44-3.42 (4H, m), 1.14 (12H, s), 0.99-0.91 (2H, m), 0.86-0.79 (2H, m). LC/MS (ESI⁺) m z=342.1 [M+1]⁺.

Step 3: To a solution 2-chloro-4-(2,4-difluorophenyl)-6,7-dimethyl-pteridine (1.0 eq, 0.70 g, 2.05 mmol) and 3-(1-cyclopropylpyrazol-4-yl)-1-methyl-5-(4,4,5,5-tetramethyl-1,3,2-dioxaborolan-2-yl)pyridin-2-one (1.0 eq, 0.70 g, 2.05 mmol) in 1,4-dioxane (15 mL) was added Cs₂CO₃ (3.0 eq, 2.0 g, 6.15 mmol), water (3 mL) and Pd(dppf)Cl₂ (0.05 eq, 208 mg, 0.26 mmol). The mixture was stirred at 80° C. until completion was indicated by TLC. The reaction mixture was cooled down to room temperature then water and EtOAc were added. The organic phase was collected, and the aqueous layer was extracted with EtOAc. The combined organic layers were washed with water, brine, dried over MgSO₄, filtered, and concentrated in vacuo. The crude material was purified by silica gel chromatography using a 80 g pre-packed column eluting with EtOAc in DCM (30 to 100%) and then MeOH in DCM (5 to 20%) to provide 3-(1-cyclopropyl-1H-pyrazol-4-yl)-5-(4-(2,4-difluorophenyl)-6,7-dimethylpteridin-2-yl)-1-methylpyridin-2(1H)-one (0.87 g, 1.8 mmol, 87% yield) as a yellow solid. ¹H NMR (400 MHz, Chloroform-d) δ_{H} 8.87 (1H, d, J=2.4 Hz), 8.78 (1H, d, J=2.4 Hz), 8.43 (1H, s), 8.08 (1H, s), 7.84-7.78 (1H, m), 7.24 (1H, s), 7.13-6.99 (2H, m), 3.74 (3H, s), 3.63 (1H, tt, J=7.3, 3.8 Hz), 2.84 (3H, s), 2.71 (3H, s), 1.19-1.11 (2H, m), 1.06-0.99 (2H, m). LC/MS (ESI⁺) m/z=486.3 [M+1]⁺.

### Synthesis of Compound I-1462

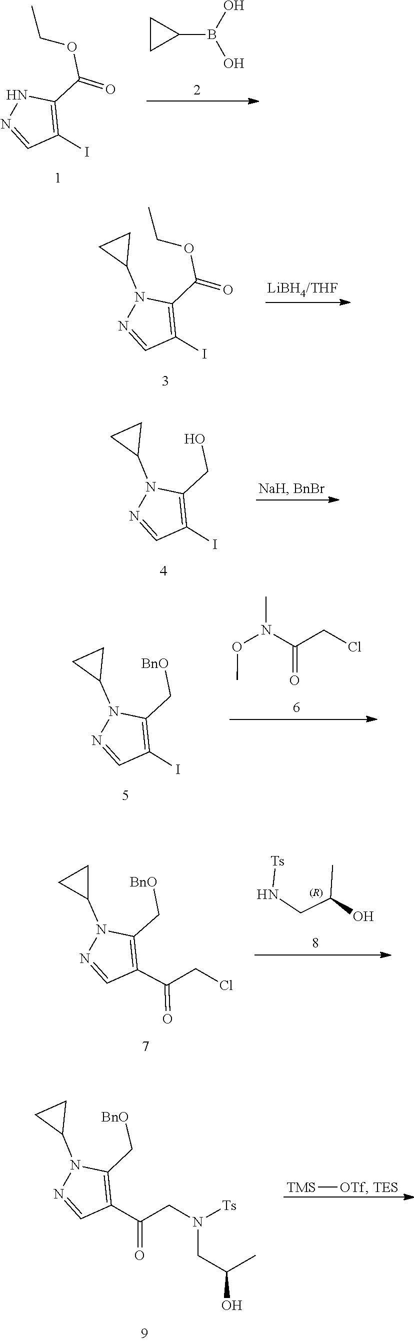

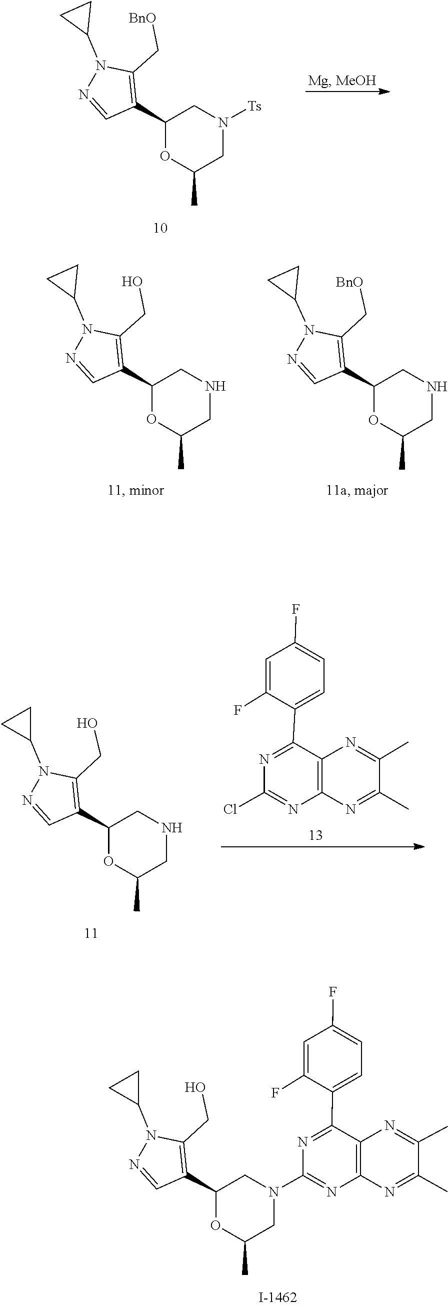 [SMILES: Cc1ccc(S(=O)(=O)N2C[C@@H](C)O[C@@H](c3cnn(C4CC4)c3COCc3ccccc3)C2)cc1 ; C[C@@H]1CNC[C@H](c2cnn(C3CC3)c2CO)O1 ; C[C@@H]1CNC[C@H](c2cnn(C3CC3)c2COCc2ccccc2)O1 ; C[C@@H]1CNC[C@H](c2cnn(C3CC3)c2CO)O1 ; Cc1nc2nc(Cl)nc(-c3ccc(F)cc3F)c2nc1C ; Cc1nc2nc(N3C[C@@H](C)O[C@@H](c4cnn(C5CC5)c4CO)C3)nc(-c3ccc(F)cc3F)c2nc1C]

Step 1: The mixture of ethyl 4-iodo-1H-pyrazole-5-carboxylate (1.00 eq, 4.00 g, 15.0 mmol), cyclopropylboronic acid (2.00 eq, 2583 mg, 30.1 mmol), Cs₂CO₃ (2.50 eq, 12216 mg, 37.6 mmol), Cu(OAc)₂ (0.840 eq, 2522 mg, 12.6 mmol) and DMAP (4.00 eq, 7337 mg, 60.1 mmol) in 1,4-Dioxane (100 mL) was stirred at 50° C. for 12 h under N₂ atmosphere. TLC (EA/PE=1/10, the desired product Rf=0.5) showed ethyl 4-iodo-1H-pyrazole-5-carboxylate was consumed and the new spot was detected. The reaction mixture was cooled to room temperature. Then EtOAc (200 mL) was added and the resulting mixture was stirred for 1 hour. After filtration, the filtrate was concentrated to dryness to give the crude product. The crude was purified by silica gel column chromatography (EA/PE=0/1 to 1/10, the desired product Rf=0.5) to give the ethyl 2-cyclopropyl-4-iodo-pyrazole-3-carboxylate (1900 mg, 6.11 mmol, 40.62% yield) as colorless oil, checked by LCMS [M+H]⁺=282.2; purity=97.5% (220 nm). Retention time=0.914 min. 1H NMR (400 MHz, CDCl₃) δ 8.02 (s, 1H), 7.71-7.63 (m, 1H), 7.11-7.03 (m, 1H), 6.99-6.94 (m, 1H), 5.26 (s, 2H), 5.19 (s, 2H).

Step 2: To the mixture of ethyl 2-cyclopropyl-4-iodo-pyrazole-3-carboxylate (1.00 eq, 1900 mg, 6.21 mmol) in THF (100 mL) was added LiBH₄ (3.00 eq, 406 mg, 18.6 mmol) at 0° C. The mixture was stirred at 40° C. for 3 h. LC-MS showed starting material was consumed completely and desired mass was detected (86%, Rt: 0.443 min; [M+H]⁺=265.0 at 220 nm). The mixture was added into sat aq NH₄Cl (200 mL). The mixture was extracted with EtOAc (200 mL*2) and the organic phase was concentrated to dryness to give a residue. The residue was used for next step directly without purification. (2-cyclopropyl-4-iodo-pyrazol-3-yl) methanol (1600 mg, 5.39 mmol, 86.88% yield) was obtained as yellow oil, checked by LCMS [M+H]⁺=265.0; purity=86.88% (220 nm). Retention time=0.443 min.

Step 3: To a solution of (2-cyclopropyl-4-iodo-pyrazol-3-yl)methanol (1.00 eq, 1000 mg, 3.79 mmol) in DMF (50 mL) was added NaH (1.50 eq, 341 mg, 5.68 mmol) at 0° C. and stirred for 0.5 h, then BnBr (1.50 eq, 0.68 mL, 5.68 mmol) was added to the reaction mixture and stirred at 25° C. for 12 h. LC-MS showed starting material was consumed completely and desired mass was detected (88%, Rt: 0.919 min; [M+H]⁺=355.1 at 220 nm). The reaction mixture was poured into 100 mL of water carefully and extracted with EA (100 mL×3), the combined organic phase was dried over Na₂SO₄ and concentrated to give a residue. The residue was purified by column chromatography (SiO2, PE:EA=10/1, the desired product Rf=0.7) and concentrated to give 5-(benzyloxymethyl)-1-cyclopropyl-4-iodo-pyrazole (750 mg, 2.09 mmol, 55.13% yield) as white solid, checked by LCMS [M+H]⁺=355.1; purity=98.6% (220 nm). Retention time=0.919 min.

Step 4: To a solution of 5-(benzyloxymethyl)-1-cyclopropyl-4-iodo-pyrazole (1.00 eq, 380 mg, 1.07 mmol) in THF (20 mL) was added i-PrMgCl LiCl (1.10 eq, 0.91 mL, 1.18 mmol) at 20° C. under N₂ atmosphere for 0.5 h, then 2-chloro-N-methoxy-N-methylacetamide (3.00 eq, 443 mg, 3.22 mmol) was added to the reaction mixture at 20° C. and stirred at 20° C. for 1 h. LCMS showed starting material was consumed completely and desired mass was detected (67%, Rt: 0.89 min; [M+H]⁺=305.3 at 254 nm). The reaction solution was quenched with saturated NH₄C1, poured into H₂O, extracted with EtOAc and evaporated under reduced pressure to give the residue. The residue was purified by column chromatography (SiO2, PE:EA=5/1, the desired product Rf=0.5) and concentrated to give 1-[5-(benzyloxymethyl)-1-cyclopropyl-pyrazol-4-yl]-2-chloro-ethanone (420 mg, 1.10 mmol, 102.76% yield) as colorless oil. [M+H]⁺: (M+H)=305.3; purity=67.8% (254 nm). Retention time=0.89 min.

Step 5: To a solution of 1-[5-(benzyloxymethyl)-1-cyclopropyl-pyrazol-4-yl]-2-chloro-ethanone (1.10 eq, 414 mg, 1.34 mmol) in acetone (20 mL) was added N-[(2R)-2-hydroxypropyl]-4-methyl-benzenesulfonamide (1.00 eq, 280 mg, 1.22 mmol), K₂CO₃ (3.00 eq, 506 mg, 3.66 mmol) and KI (1.00 eq, 203 mg, 1.22 mmol), the reaction mixture was stirred at 25° C. for 12 h. LCMS showed starting material was consumed completely and desired mass was detected (15%, Rt: 0.796 min; [M+Na]⁺=520.1 at 254 nm). The reaction was concentrated to give a residue. The residue was purified by column chromatography (SiO2, PE:EA=1/1, the desired product Rf=0.2) and concentrated to give N-[2-[5-(benzyloxymethyl)-1-cyclopropyl-pyrazol-4-yl]-2-oxo-ethyl]-4-methyl-N-[(2S)-2-hydroxypropyl]benzenesulfonamide (435 mg, 0.542 mmol, 44.39% yield) as white solid, checked by LCMS [M+Na]⁺=520.1; purity=90.4% (UV 254 nm). Retention time=0.901 min.

Step 6: To a solution of N-[2-[5-(benzyloxymethyl)-1-cyclopropyl-pyrazol-4-yl]-2-oxo-ethyl]-N-[(2R)-2-hydroxypropyl]-4-methyl-benzenesulfonamide (1.00 eq, 435 mg, 0.874 mmol), TES (10.0 eq, 2.7 mL, 8.74 mmol) in DCM (20 mL) was added TMSOTf (8.00 eq, 1.3 mL, 6.99 mmol) at 0° C., the reaction mixture was stirred at 30° C. for 12 h. LCMS showed starting material was consumed completely and desired mass was detected (36%, Rt: 0.951 min; [M+H]⁺=482.3 at 220 nm). The reaction was concentrated to give a residue. The residue was prep-HPLC (column: Waters Xbridge 150*25 mm * 5 um; mobile phase: [water (NH₄HCO₃)-ACN]; B %: 50%-80%, 8 min) and lyophilized to give [2-cyclopropyl-4-[(2S,6R)-6-methyl-4-(p-tolylsulfonyl) morpholin-2-yl] pyrazol-3-yl] methanol (90 mg, 0.207 mmol, 23.67% yield) as white solid. [M+H]⁺=482.2; purity=25.4% (UV 254 nm). Retention time=0.933 min.

Step 7: To a solution of (2S,6R)-2-[5-(benzyloxymethyl)-1-cyclopropyl-pyrazol-4-yl]-6-methyl-4-(p-tolylsulfonyl)morpholine (1.00 eq, 80 mg, 0.166 mmol) in Methanol (16 mL) was added Mg (Chips) (20.0 eq, 80 mg, 3.32 mmol) and Mg (powder) (20.0 eq, 80 mg, 3.32 mmol), the reaction was stirred at 80° C. for 12 h under Ar atmosphere. LCMS showed 50% starting material was remained and 40% desired mass was detected. Then Mg (Chips) (20.0 eq, 80 mg, 3.32 mmol) and Mg (powder) (20.0 eq, 80 mg, 3.32 mmol) was added to the reaction and stirred at 80° C. for 12 h under Ar atmosphere. LCMS showed 25% starting material was remained and desired mass was detected (4%, Rt: 0.458 min; [M+H]⁺=238.1 at 220 nm). The mixture was filtered and the filtrate was concentrated in vacuo. The residue was purified by prep-HPLC (column: Shim-pack C18 150*25*10 um; mobile phase: [water (FA)-ACN]; B %: 2%-32%, 10 min) and concentrated to give [2-cyclopropyl-4-[(2S,6R)-6-methylmorpholin-2-yl]pyrazol-3-yl]methanol (3.0 mg, 0.0126 mmol, 7.61% yield) as white solid. [M+H]⁺=238.1; purity=3.6% (UV 220 nm). Retention time=0.455 min.

Step 8: To a solution of [2-cyclopropyl-4-[(2S,6R)-6-methylmorpholin-2-yl]pyrazol-3-yl]methanol (1.00 eq, 3.0 mg, 0.0126 mmol) in DMSO (1 mL) was added 2-chloro-4-(2,4-difluorophenyl)-6,7-dimethyl-pteridine (1.50 eq, 5.8 mg, 0.0190 mmol) and DIEA (3.00 eq, 4.9 mg, 0.0379 mmol), the reaction was stirred at 100° C. for 1 h. LCMS showed starting material was consumed completely and desired mass was detected (41%, Rt: 0.872 min; [M+H]⁺=508.1 at 220 nm). The reaction was purified by prep-HPLC (column: Unisil 3-100 C18 Ultra 150*50 mm*3 um; mobile phase: [water (FA)-ACN]; B %: 6%-36%, 7 min), the solution was lyophilized to give [2-cyclopropyl-4-[(2S,6R)-4-[4-(2,4-difluorophenyl)-6,7-dimethyl-pteridin-2-yl]-6-methyl-morpholin-2-yl]pyrazol-3-yl]methanol (1.6 mg, 0.00315 mmol, 24.94% yield) as white solid. LCMS: (M+H)=508.1; purity=96.8% (UV 220 nm). Retention time=0.943 min. ¹H NMR (400 MHz, CDCl₃) δ 7.76-7.65 (m, 1H), 7.42 (s, 1H), 7.07-6.93 (m, 2H), 5.26-5.09 (m, 1H), 5.04-5.01 (m, 1H), 4.90-4.77 (m, 2H), 4.74-4.70 (m, 1H), 3.94-3.88 (m, 1H), 3.51-3.42 (m, 1H), 3.25-3.17 (m, 1H), 2.91-2.83 (m, 1H), 2.71 (s, 3H), 2.59 (s, 3H), 1.35 (d, J=6.1 Hz, 3H), 1.23-1.20 (m, 2H), 1.09-1.06 (m, 2H).

### Synthesis of Compounds I-1467 and I-1524

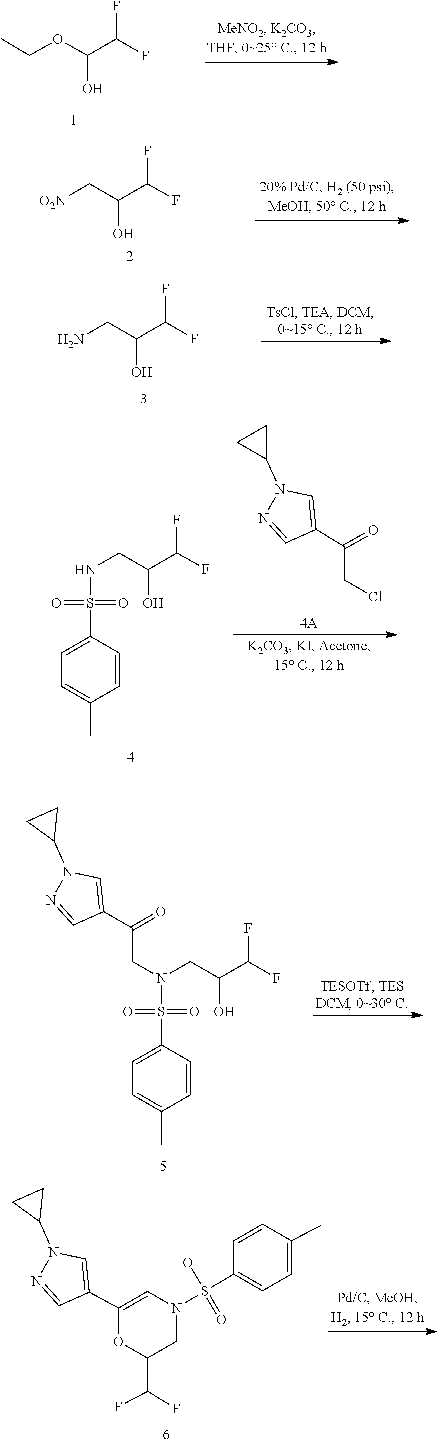

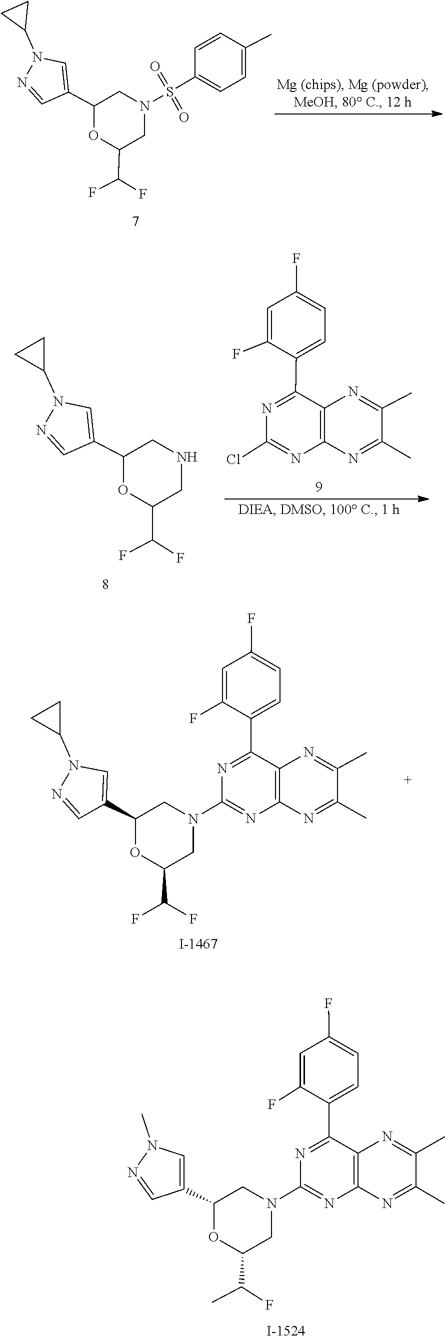

1: To a colorless mixture of 1-ethoxy-2,2-difluoro-ethanol (1.00 eq, 4.00 g, 31.7 mmol) in THF (160 mL) was added K₂CO₃ (0.1000 eq, 438 mg, 3.17 mmol), then nitromethane (1.50 eq, 2904 mg, 47.6 mmol) was added at 0° C., then the mixture was stirred at 15° C. for 12 hr to give yellow solution. TLC (PE:EA=2:1, phosphomolybdic acid, Rf=0.5) showed the starting material was consumed completely and new spots was found. The mixture was poured into 1N HCl (160 mL) and extracted by MTBE (500 mL×3), the combined organic layers were washed with brine, dried over anhydrous sodium sulfate, filtered and concentrated in vacuo. 1,1-difluoro-3-nitro-propan-2-ol (4.50 g, 25.5 mmol, 80.45% yield) was obtained as yellow oil and used to the next step.

Step 2: To a mixture of 1,1-difluoro-3-nitro-propan-2-ol (1.00 eq, 1.00 g, 7.09 mmol) in Methanol (20 mL) was added 10% Pd/C (1.00 eq, 100 mg, 7.09 mmol). Then the reaction mixture was degassed with H₂ for 3 times. The reaction mixture was stirred at 50° C. for 12 hr under H₂ atmosphere (50 psi). TLC (PE:EA=2:1, I₂, Rf=0.01) showed the starting material was consumed completely and new spot was found. The solution was filtered under N₂ atmosphere, the filtrate was concentrated under reduced pressure to give yellow solid, 3-amino-1,1-difluoro-propan-2-ol (400 mg, 2.16 mmol, 30.4% yield) was obtained as yellow solid.

Step 3: To a mixture of 3-amino-1,1-difluoro-propan-2-ol (1.00 eq, 350 mg, 3.15 mmol) in DCM (10 mL) was added TEA (3.00 eq, 0.82 mL, 9.45 mmol), then TsCl (1.00 eq, 601 mg, 3.15 mmol) was added at 0° C., then the mixture was stirred at 15° C. for 12 hr. LCMS showed the starting material was consumed completely and 68% desired MS (Rt: 0.587 min; [M+H]⁺=265.8 at 220 nm) was found. TLC (PE:EA=1:1, Rf=0.5) showed the starting material was consumed completely and new spots was found. The work up and purification was combined with. The combined mixture was poured into water (30 mL) and extracted by ethyl acetate (40 mL×3), the combined organic layers were washed with brine, dried over anhydrous sodium sulfate, filtered and concentrated in vacuo. The solution was purified by column on silica (5 g SiO2 cartridge, PE:EA=32%, detection at 254 nm, Rf=0.5) and concentrated under reduced pressure to give crude N-(3,3-difluoro-2-hydroxy-propyl)-4-methyl-benzenesulfonamide (1.40 g, 5.01 mmol, 159.13% yield) as white solid. [M+H]⁺=265.8; purity=68% (220 nm). Retention time=0.587 min. ¹H NMR (400 MHz, CDCl3) δ=7.77 (d, J=8.3 Hz, 2H), 7.35 (d, J=8.0 Hz, 2H), 5.87 (d, J=4.4 Hz, 1H), 5.73 (d, J=4.3 Hz, 1H), 5.59 (d, J=4.3 Hz, 1H), 4.88 (br d, J=5.5 Hz, 1H), 3.98-3.85 (m, 1H), 3.27 (ddd, J=3.6, 7.3, 13.8 Hz, 1H), 3.15-3.07 (m, 1H), 2.70 (br d, J=4.9 Hz, 1H), 2.45 (s, 3H).

Step 4: To a mixture of N-(3,3-difluoro-2-hydroxy-propyl)-4-methyl-benzenesulfonamide (1.00 eq, 700 mg, 2.64 mmol) in acetone (20 mL) was added 2-chloro-1-(1-cyclopropylpyrazol-4-yl)ethanone (1.00 eq, 487 mg, 2.64 mmol), KI (1.00 eq, 438 mg, 2.64 mmol), K₂CO₃ (3.00 eq, 1094 mg, 7.92 mmol), then the mixture was stirred at 15° C. for 12 hr. LCMS showed the starting material was consumed completely and 71% desired MS (Rt: 0.829 min; [M+H]⁺=414.1 at 220 nm) was found. The work up and purification was combined with. The combined mixture was poured into water (50 mL) and extracted by ethyl acetate (50 mL×3), the combined organic layers were washed with brine, dried over anhydrous sodium sulfate, filtered and concentrated in vacuo. The solution was purified by column on silica (2 g SiO₂ cartridge, PE:EA=45%, detection at 254 nm, Rf=0.3) and concentrated under reduced pressure to give N-[2-(1-cyclopropylpyrazol-4-yl)-2-oxo-ethyl]-N-(3,3-difluoro-2-hydroxy-propyl)-4-methyl-benzenesulfonamide (650 mg, 1.49 mmol, 56.60% yield) as colorless oil. [M+H]⁺=414.1; purity=71% (220 nm). Retention time=0.829 min. ¹H NMR (400 MHz, CDCl3) δ=8.05 (s, 1H), 7.91 (s, 1H), 7.73 (d, J=8.3 Hz, 2H), 7.35 (d, J=8.0 Hz, 2H), 5.92 (d, J=3.4 Hz, 1H), 5.78 (d, J=3.3 Hz, 1H), 5.64 (d, J=3.4 Hz, 1H), 4.52 (s, 2H), 4.35-4.22 (m, 1H), 4.08-3.95 (m, 1H), 3.67 (tt, J=3.8, 7.3 Hz, 1H), 3.52-3.44 (m, 1H), 3.38-3.30 (m, 1H), 2.46 (s, 3H), 1.21-1.15 (m, 2H), 1.15-1.09 (m, 2H).

Step 5: To a colorless mixture of N-[2-(1-cyclopropylpyrazol-4-yl)-2-oxo-ethyl]-N-(3,3-difluoro-2-hydroxy-propyl)-4-methyl-benzenesulfonamide (1.00 eq, 300 mg, 0.726 mmol) in DCM (5 mL) was added TES (5.00 eq, 832 mg, 3.63 mmol) and TMSOTf (5.00 eq, 0.66 mL, 3.63 mmol) at 0° C., then the mixture was stirred at 30° C. for 12 hr to give colorless mixture. LCMS showed the starting material was consumed completely and 70.9% desired MS (Rt: 0.786 min; [M+H]⁺=395.9 at 220 nm) was found. The mixture was quenched with sat. aq. NaHCO₃ (20 mL) and extracted with EtOAc (30 mL×3), the combined organic layers were washed with brine, dried over anhydrous sodium sulfate, filtered and concentrated in vacuo. 6-(1-cyclopropylpyrazol-4-yl)-2-(difluoromethyl)-4-(p-tolylsulfonyl)-2,3-dihydro-1,4-oxazine (280 mg, 0.496 mmol, 68.31% yield) was obtained as white solid. [M+H]⁺=395.9; purity=70.9% (220 nm). Retention time=0.786 min.

Step 6: To a colorless mixture of 6-(1-cyclopropylpyrazol-4-yl)-2-(difluoromethyl)-4-(p-tolylsulfonyl)-2,3-dihydro-1,4-oxazine (1.00 eq, 230 mg, 0.582 mmol) in methanol (5 mL) was added 10% Pd/C (1.00 eq, 25 mg, 0.582 mmol). Then the reaction mixture was degassed with H₂ for 3 times. The reaction mixture was stirred at 30° C. for 2 hr under H₂ atmosphere (15 psi). LCMS showed 57% starting material still remained with 38% desired mass (397.9 [M+H]⁺, ESI pos). 10% Pd/C (1.00 eq, 50 mg, 0.582 mmol) was added, then the mixture was stirred at 30° C. for 6 hr under H₂ atmosphere (15 psi). LCMS showed the starting material was consumed completely and 95.3% desired MS (Rt: 0.952 min; [M+H]⁺=398.1 at 220 nm) was found. The solution was filtered under N₂ atmosphere, the filtrate was concentrated under reduced pressure to give a residue. 2-(1-cyclopropylpyrazol-4-yl)-6-(difluoromethyl)-4-(p-tolylsulfonyl)morpholine (230 mg, 0.550 mmol, 94.52% yield) was obtained as colorless oil. [M+H]⁺=398.1; purity=95.3% (220 nm). Retention time=0.952 min.

Step 7: To a colorless mixture of 2-(1-cyclopropylpyrazol-4-yl)-6-(difluoromethyl)-4-(p-tolylsulfonyl)morpholine (1.00 eq, 230 mg, 0.579 mmol) in methanol (5 mL) was added Mg (chips) (10.0 eq, 139 mg, 5.79 mmol) and Mg (powder) (10.0 eq, 139 mg, 5.79 mmol), then the mixture was stirred at 80° C. for 12 hr under N₂ atmosphere to give a white suspension. LCMS showed the starting material was consumed completely and desired MS (Rt: 0.332 min; [M+H]⁺=244.5 at 220 nm) was found. The reaction mixture was cooled to room temperature. The reaction mixture was filtered, the filtrate was concentrated under reduced pressure to give white solid. 2-(1-cyclopropylpyrazol-4-yl)-6-(difluoromethyl)morpholine (200 mg, 0.493 mmol, 85.25% yield) was obtained as white solid. [M+H]⁺=244.5; purity=90% (220 nm). Retention time=0.332 min.

Step 8: To a brown mixture of 2-chloro-4-(2,4-difluorophenyl)-6,7-dimethyl-pteridine (1.00 eq, 177 mg, 0.576 mmol) in DMSO (5 mL) was added 2-(1-cyclopropylpyrazol-4-yl)-6-(difluoromethyl)morpholine (1.00 eq, 140 mg, 0.576 mmol) and DIEA (3.00 eq, 223 mg, 1.73 mmol), then the mixture was stirred at 100° C. for 1 hr to give brown solution. LCMS showed the starting material was consumed completely and 74% desired MS (Rt: 1.049 min; [M+H]⁺=514.0 at 220 nm) was found. The reaction mixture was cooled to room temperature. The mixture was poured into water (30 mL) and extracted by ethyl acetate (50 mL×3), the combined organic layers were washed with brine, dried over anhydrous sodium sulfate, filtered and concentrated in vacuo. The solution was purified by prep-HPLC (column: Waters Xbridge 150*25 mm*5 um; mobile phase: water (NH₄HCO₃)-ACN; B %: 53%-83%, 8 min), the purified solution was lyophilized to give yellow solid. The solution was repurified by SFC (column: DAICEL CHIRALPAK IC (250 mm*30 mm, 10 um); mobile phase: ACN/MeOH (0.1% NH₃H₂O); B %: 30%-30%, 3.6 min), the purified solution was lyophilized to give (2S,6S)-2-(1-cyclopropylpyrazol-4-yl)-6-(difluoromethyl)-4-[4-(2,4-difluorophenyl)-6,7-dimethyl-pteridin-2-yl]morpholine (22 mg, 0.0423 mmol, 7.35% yield) as yellow solid. (2R,6R)-2-(1-cyclopropylpyrazol-4-yl)-6-(difluoromethyl)-4-[4-(2,4-difluorophenyl)-6,7-dimethyl-pteridin-2-yl]morpholine (22 mg, 0.0420 mmol, 7.31% yield) was obtained as yellow solid. LCMS (peak 1 in SFC) [M+H]⁺=514.1; purity=100% (220 nm). Retention time=1.047 min. ee. =99.58%. ¹H NMR (400 MHz, CDCl3) δ=7.77-7.68 (m, 1H), 7.56 (s, 2H), 7.10-7.03 (m, 1H), 6.99 (dt, J=2.3, 9.5 Hz, 1H), 6.03-5.99 (m, 1H), 5.87 (d, J=3.7 Hz, 1H), 5.73 (d, J=3.7 Hz, 1H), 5.16 (br d, J=12.5 Hz, 2H), 4.67 (dd, J=2.4, 10.9 Hz, 1H), 4.05-3.93 (m, 1H), 3.59 (tt, J=3.7, 7.3 Hz, 1H), 3.14 (ddd, J=7.6, 11.1, 13.4 Hz, 2H), 2.74 (s, 3H), 2.62 (s, 3H), 1.17-1.10 (m, 2H), 1.08-0.98 (m, 2H). Peak 2 in SFC) [M+H]⁺=514.0; purity=100% (220 nm). Retention time=1.046 min. ee.=96.226%. ¹H NMR (400 MHz, CDCl3) δ=7.78-7.68 (m, 1H), 7.56 (s, 2H), 7.06 (dt, J=1.9, 8.2 Hz, 1H), 6.98 (dt, J=2.3, 9.5 Hz, 1H), 6.01 (d, J=3.7 Hz, 1H), 5.87 (d, J=3.7 Hz, 1H), 5.73 (d, J=3.7 Hz, 1H), 5.16 (br d, J=13.4 Hz, 2H), 4.67 (dd, J=2.4, 10.9 Hz, 1H), 4.05-3.93 (m, 1H), 3.59 (tt, J=3.7, 7.3 Hz, 1H), 3.14 (ddd, J=7.6, 11.1, 13.4 Hz, 2H), 2.74 (s, 3H), 2.62 (s, 3H), 1.16-1.08 (m, 2H), 1.07-0.99 (m, 2H).

### Synthesis of Compound I-1472

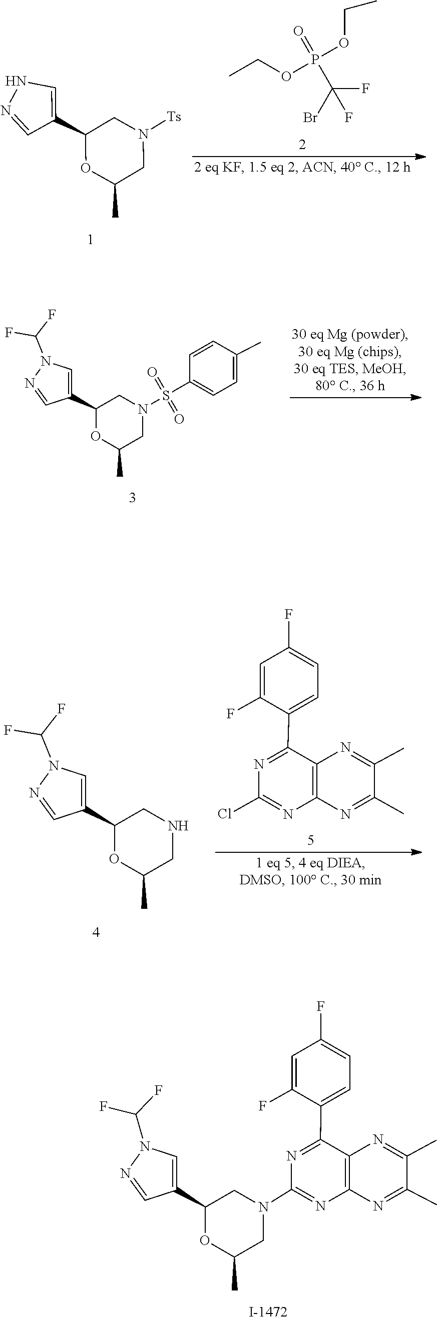 [SMILES: Cc1ccc(S(=O)(=O)N2C[C@@H](C)O[C@@H](c3cn[nH]c3)C2)cc1 ; CCOP(=O)(OCC)C(F)(F)Br ; Cc1ccc(S(=O)(=O)N2C[C@@H](C)O[C@@H](c3cnn(C(F)F)c3)C2)cc1 ; C[C@@H]1CNC[C@H](c2cnn(C(F)F)c2)O1 ; Cc1nc2nc(Cl)nc(-c3ccc(F)cc3F)c2nc1C ; Cc1nc2nc(N3C[C@@H](C)O[C@@H](c4cnn(C(F)F)c4)C3)nc(-c3ccc(F)cc3F)c2nc1C]

Step 1: To a solution of (2R,6S)-2-methyl-4-(p-tolylsulfonyl)-6-(1H-pyrazol-4-yl)morpholine (1.00 eq, 500 mg, 1.56 mmol), KF (2.00 eq, 181 mg, 3.11 mmol) and 1-[[bromo(difluoro)methyl]-ethoxy-phosphoryl]oxyethane (1.50 eq, 623 mg, 2.33 mmol) in MeCN (15 mL) then the mixture was stirred at 40° C. for 12 hours. LCMS (5-95AB/1.5 min): RT=0.630 min, 372.0=[M+H]⁺, ESI⁺ showed 45.3% of desired product and RT=0.543 min, 322.0=[M+H]⁺, ESI⁺ showed 54% of starting material. Then the reaction mixture was stirred at 50° C. for 5 hours. LCMS (5-95AB/1.5 min): RT=0.629 min, 372.1=[M+H]⁺, ESI⁺ showed 90.0% of desired product. The reaction was diluted with water (50 mL) and then extracted with ethyl acetate (50 mL*3). The combined organic layers were washed with brine, dried over Na₂SO₄, filtered and concentrated under reduced pressure to give a residue. The residue was purified by flash chromatography on silica gel eluting with PE/EtOAc (4:1) (TLC, PE: EtOAc=1:1, Rf=0.60) to afford (2S,6R)-2-[1-(difluoromethyl)pyrazol-4-yl]-6-methyl-4-(p-tolylsulfonyl)morpholine (284 mg, 0.765 mmol, 49.15% yield) as colorless gum. [M+H]⁺=372.1; purity=100% (220 nm). Retention time=0.725 min. ¹H NMR (400 MHz, CDCl3) δ ppm 1.20-1.25 (m, 3H) 2.04-2.08 (m, 1H) 2.17-2.27 (m, 1H) 2.43-2.50 (m, 3H) 3.67 (br dd, J=11.32, 1.94 Hz, 1H) 3.75-3.83 (m, 1H) 3.84-3.94 (m, 1H) 4.72 (dd, J=10.44, 2.44 Hz, 1H) 6.98-7.20 (m, 1H) 7.37 (br d, J=8.00 Hz, 2H) 7.60-7.70 (m, 3H) 7.76-7.81 (m, 1H).

Step 2: To the mixture of (2S,6R)-2-[1-(difluoromethyl)pyrazol-4-yl]-6-methyl-4-(p-tolylsulfonyl)morpholine (1.00 eq, 50 mg, 0.135 mmol) and Et₃SiH (46.5 eq, 1.0 mL, 6.26 mmol) in Methanol (10 mL) was added Mg powder (30.0 eq, 97 mg, 4.04 mmol) and Mg chips (30.0 eq, 97 mg, 4.04 mmol) at 25° C. and the reaction mixture was stirred for 12 hours at 80° C. under N₂ atmosphere. LCMS (0-60AB/1.5 min): RT=0.353 min, 218.1=[M+H]⁺, ESI⁺ showed 16.7% of desired product and RT=0.935 min, 372.1=[M+H]⁺, ESI⁺ showed 45% of starting material. Then Mg powder (20.0 eq, 65 mg, 2.69 mmol) and Mg chips (20.0 eq, 65 mg, 2.69 mmol) was added and the reaction mixture was stirred at 80° C. for 12 hours under N₂ atmosphere. LCMS (0-60AB/1.5 min): RT=0.381 min, 218.1=[M+H]⁺, ESI⁺ showed 30.4% of desired product. The reaction mixture was filtered through a pad of celite. The filter cake was washed with MeOH (50 mL). The filtrate was concentrated under reduced pressure to afford crude product (2S,6R)-2-[1-(difluoromethyl)pyrazol-4-yl]-6-methyl-morpholine (50 mg, 0.113 mmol, 83.78% yield) as white solid. It was used to next step without further purification.

Step 3: To a solution of (2S,6R)-2-[1-(difluoromethyl)pyrazol-4-yl]-6-methyl-morpholine (1.00 eq, 50 mg, 0.113 mmol) and 2-chloro-4-(2,4-difluorophenyl)-6,7-dimethyl-pteridine (1.00 eq, 47 mg, 0.113 mmol) in DMSO (2 mL) was added DIEA (4.00 eq, 58 mg, 0.451 mmol) at 25° C. Then the reaction mixture was stirred at 100° C. for 30 min. LCMS (5-95AB/1.5 min): RT=0.793 min, 488.1=[M+H]⁺, ESI⁺ showed 39.2% of desired product. The reaction was diluted with water (20 mL) and then extracted with ethyl acetate (20 mL*3). The combined organic layers were washed with brine, dried over Na₂SO₄, filtered and concentrated under reduced pressure to give a residue. The residue was purified by prep-HPLC (Column, [Unisil 3-100 C18 Ultra 150*50 mm*3 um]; mobile phase: [ACN] and [H₂O](conditions: [water (0.225% FA)-ACN], B %: 41%-71%; Detector, UV 254 nm. RT: [7 min]) to afford (2S,6R)-2-[1-(difluoromethyl)pyrazol-4-yl]-4-[4-(2,4-difluorophenyl)-6,7-dimethyl-pteridin-2-yl]-6-methyl-morpholine (20 mg, 0.0415 mmol, 36.77% yield) as yellow solid. [M+H]⁺=488.1; purity=98.9% (220 nm). Retention time=0.789 min; ¹H NMR (400 MHz, CDCl3) δ ppm 1.35 (d, J=6.25 Hz, 3H) 2.61 (s, 3H) 2.73 (s, 3H) 2.82-2.91 (m, 1H) 3.06 (dd, J=13.32, 10.94 Hz, 1H) 3.80-3.92 (m, 1H) 4.68 (dd, J=10.88, 2.38 Hz, 1H) 4.95-5.06 (m, 1H) 5.08-5.23 (m, 1H) 6.98 (td, J=9.54, 2.31 Hz, 1H) 7.04 (s, 1H) 7.04-7.09 (m, 1H) 7.19 (s, 1H) 7.34 (s, 1H) 7.68-7.78 (m, 2H) 7.91 (s, 1H).

### Synthesis of Compound I-1477

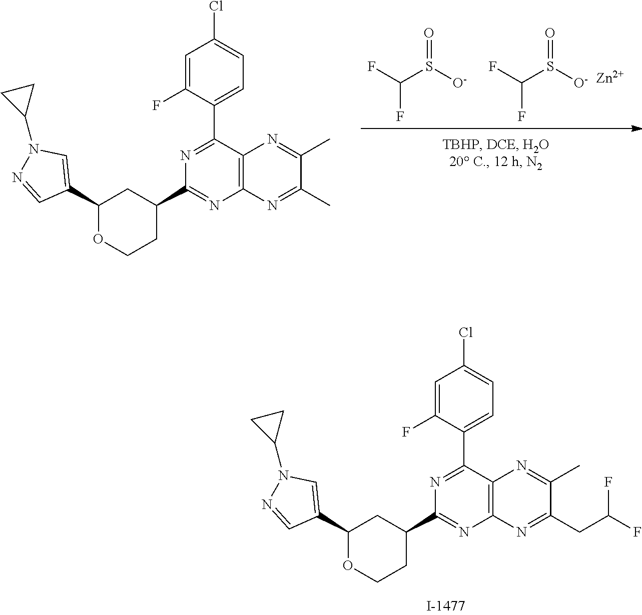 [SMILES: Cc1nc2nc([C@H]3CCO[C@@H](c4cnn(C5CC5)c4)C3)nc(-c3ccc(Cl)cc3F)c2nc1C ; O=S([O-])C(F)F ; O=S([O-])C(F)F ; [Zn+2] ; Cc1nc2c(-c3ccc(Cl)cc3F)nc([C@H]3CCO[C@@H](c4cnn(C5CC5)c4)C3)nc2nc1CC(F)F]

To a solution of 4-(4-chloro-2-fluoro-phenyl)-2-[(2R,4S)-2-(1-cyclopropylpyrazol-4-yl)tetrahydropyran-4-yl]-6,7-dimethyl-pteridine (1.00 eq, 150 mg, 0.313 mmol) and zinc difluoromethanesulfinate (4.00 eq, 368 mg, 1.25 mmol) in DCE (2 mL) and Water (0.8 mL) at 20° C. was added tert-butylhydroperoxide (7.00 eq, 197 mg, 2.19 mmol) with vigorous stirring and bubbled with N₂ for 30 seconds. The reaction solution was stirred at 20° C. for 1 hr. LCMS showed most of the starting material still remained. Then the reaction solution was stirred at 20° C. for 12 hrs. LCMS showed most of the starting material still remained and desired MS (529.2 [M+H]⁺, RT=1.008 min) was detected. Then the reaction solution was stirred at 30° C. for another 12 hrs. LCMS showed 67% of starting material still remained and 19% desired MS (529.2 [M+H]⁺, RT=1.018 min) was detected. The mixture was poured into 30 mL ice saturation Na₂SO₃ solution, extracted with EA (15 mL*3), the combine organic layers was dried over anhydrous Na₂SO₄, filtered and concentrated under reduced pressure to give the residue. The residue was purified by prep-TLC (DCM:MeOH=20:1, Rf=0.4) to give the residue (40 mg, 39% purity in LCMS). The residue was purified by prep-HPLC (flow: 25 mL/min; gradient: from 40-70% water (0.1% FA)-ACN over 10 min; column: Shim-pack C18 150*25*10 um) and lyophilized to afford 4-(4-chloro-2-fluoro-phenyl)-2-[(2R,4S)-2-(1-cyclopropylpyrazol-4-yl)tetrahydropyran-4-yl]-7-(2,2-difluoroethyl)-6-methyl-pteridine (4.9 mg, 0.00741 mmol, 2.37% yield) as yellow solid. [M+H]⁺=529.3; purity=98.819% (220 nm). Retention time=0.987 min. ¹H NMR (400 MHz, CDCl3) δ=7.70 (t, J=7.6 Hz, 1H), 7.49 (s, 2H), 7.38-7.28 (m, 2H), 6.75-6.42 (m, 1H), 4.56 (dd, J=1.6, 11.2 Hz, 1H), 4.30-4.23 (m, 1H), 3.81 (dt, J=2.8, 11.6 Hz, 1H), 3.65 (dt, J=4.8, 15.2 Hz, 2H), 3.56 (td, J=3.6, 10.8 Hz, 2H), 2.79 (s, 3H), 2.47-2.37 (m, 1H), 2.25-2.14 (m, 3H), 1.12-1.07 (m, 2H), 1.01-0.95 (m, 2H).

### Synthesis of Compound I-1485

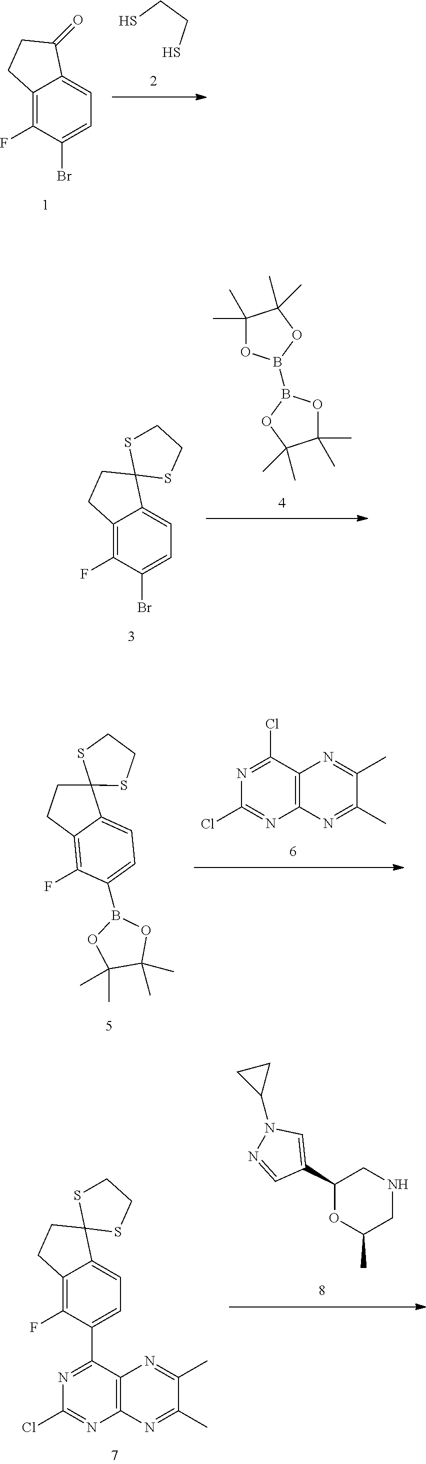

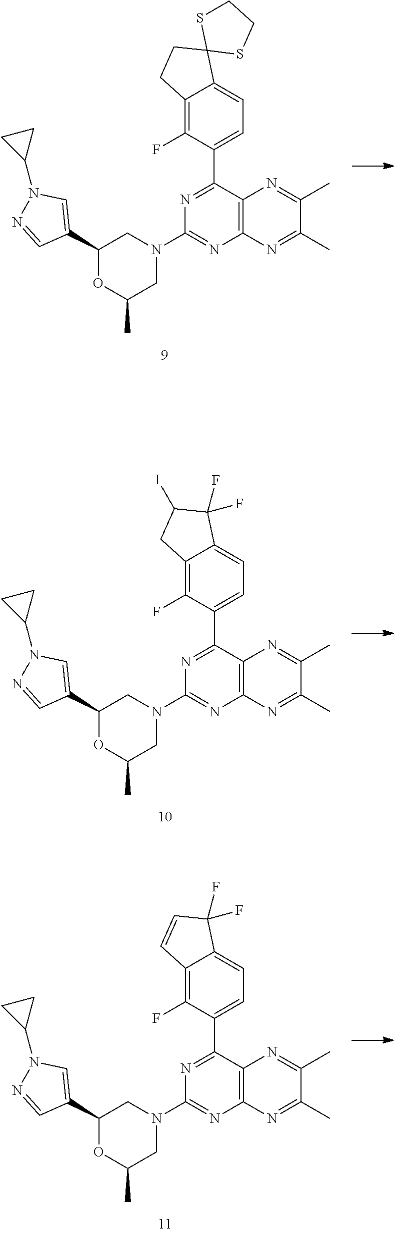 [SMILES: Cc1nc2nc(N3C[C@@H](C)O[C@@H](c4cnn(C5CC5)c4)C3)nc(-c3ccc4c(c3F)CCC43SCCS3)c2nc1C ; Cc1nc2nc(N3C[C@@H](C)O[C@@H](c4cnn(C5CC5)c4)C3)nc(-c3ccc4c(c3F)CC(I)C4(F)F)c2nc1C ; Cc1nc2nc(N3C[C@@H](C)O[C@@H](c4cnn(C5CC5)c4)C3)nc(-c3ccc4c(c3F)C=CC4(F)F)c2nc1C]

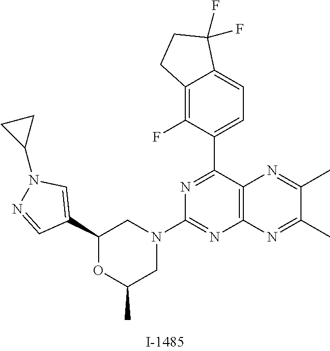 [SMILES: Cc1nc2nc(N3C[C@@H](C)O[C@@H](c4cnn(C5CC5)c4)C3)nc(-c3ccc4c(c3F)CCC4(F)F)c2nc1C]

Step 1: To a solution of 5-bromo-4-fluoro-indan-1-one (1.00 eq, 1000 mg, 4.37 mmol) and TsOH (0.200 eq, 150 mg, 0.873 mmol) in Toluene (10 mL) was added ethane-1,2-dithiol (1.02 eq, 419 mg, 4.45 mmol) and then the mixture was stirred for 16 h at 100° C. LCMS showed the major peak formed but without desired MS. The reaction solution was added NaOH (aq. 1 M)(20 mL) and then extracted with ethyl acetate (10 mL*3) and the organics washed with 10 mL saturated brine solution. The organics were then separated and dried (Na₂SO₄) before concentration to dryness. The crude was then purified by silica gel column (PE/EA=5/1) to give 5′-bromo-4′-fluoro-spiro[1,3-dithiolane-2,1′-indane](1250 mg, 4.10 mmol, 93.80% yield) as white solid. ¹H NMR (400 MHz, CDCl3) δ ppm 2.73 (t, J=6.69 Hz, 2H) 3.02 (t, J=6.69 Hz, 2H) 3.40-3.59 (m, 4H) 7.22 (d, J=8.13 Hz, 1H) 7.42 (dd, J=8.00, 6.38 Hz, 1H).

Step 2: To a solution of 5′-bromo-4′-fluoro-spiro[1,3-dithiolane-2,1′-indane] (1.00 eq, 1250 mg, 4.10 mmol) in 1,4-Dioxane (20 mL) was added KOAc (2.50 eq, 1005 mg, 10.2 mmol) and 4,4,5,5-tetramethyl-2-(4,4,5,5-tetramethyl-1,3,2-dioxaborolan-2-yl)-1,3,2-dioxaborolane (1.50 eq, 1560 mg, 6.14 mmol), then Pd(dppf)Cl₂·CH₂Cl₂ (0.100 eq, 332 mg, 0.410 mmol) was added to the mixture under N₂. And then the mixture was stirred for 16 h at 100° C. LCMS showed starting material was consumed and the major peak showed desired MS (M+H)⁺=353.1, purity=23.96%, uv=220 nm. Retention time=0.752 min. The reaction solution was poured into water (50 mL) and then extracted with ethyl acetate (20 mL*2) and the organics washed with 10 mL saturated brine solution. The organics were then separated and dried (Na₂SO₄) before concentration to dryness. The crude was then purified by silica gel column (PE/EA=5/1, Rf=0.5) to give 2-(4′-fluorospiro[1,3-dithiolane-2,1′-indane]-5′-yl)-4,4,5,5-tetramethyl-1,3,2-dioxaborolane (1300 mg, 3.69 mmol, 90.11% yield) as white solid. ¹H NMR (400 MHz, CDCl₃) δ ppm 1.27 (s, 11H) 2.67-2.73 (m, 2H) 2.98 (t, J=6.75 Hz, 2H) 3.42-3.49 (m, 2H) 3.50-3.57 (m, 2H) 7.32 (d, J=7.50 Hz, 1H) 7.60-7.68 (m, 1H). MS (M+H)⁺=353.1, purity=23.9%, uv=220 nm. Retention time=0.752 min.

Step 3: To a solution of 2-(4′-fluorospiro[1,3-dithiolane-2,1′-indane]-5′-yl)-4,4,5,5-tetramethyl-1,3,2-dioxaborolane (1.00 eq, 1400 mg, 3.97 mmol) in Water (2 mL) and Toluene (20 mL) was added K₃PO₄ (3.00 eq, 2527 mg, 11.9 mmol) and PdCl₂ (amphos) (0.0500 eq, 141 mg, 0.199 mmol) under N₂ and then the mixture was stirred for 16 h at 55° C. LCMS showed raw material was consumed and the major peak showed desired MS (M+H)⁺=419.0, purity=23.3%, uv=220 nm. Retention time=0.985 min. The reaction solution was poured into water (50 mL) and then extracted with ethyl acetate (20 mL*2) and the organics washed with 10 mL saturated brine solution. The organics were then separated and dried (Na₂SO₄) before concentration to dryness. The crude was then purified by silica gel column (PE/EA=1/1, Rf=0.5) to give 2-chloro-4-(4′-fluorospiro[1,3-dithiolane-2,1′-indane]-5′-yl)-6,7-dimethyl-pteridine (950 mg, 2.27 mmol, 57.06% yield) as red solid. ¹H NMR (400 MHz, CDCl3) δ ppm 2.76 (s, 3H) 2.81 (t, J=6.69 Hz, 2H) 2.84-2.87 (m, 3H) 3.09 (t, J=6.69 Hz, 2H) 3.47-3.54 (m, 2H) 3.55-3.62 (m, 2H) 7.52 (d, J=7.88 Hz, 1H) 7.61-7.70 (m, 1H). LCMS (M+H)⁺=419.0, purity=23.3%, uv=220 nm. Retention time=0.985 min.

Step 4: To a solution of 2-chloro-4-(4′-fluorospiro[1,3-dithiolane-2,1′-indane]-5′-yl)-6,7-dimethyl-pteridine (1.00 eq, 950 mg, 2.27 mmol) and (6R)-2-(1-cyclopropylpyrazol-4-yl)-6-methyl-morpholine (1.50 eq, 705 mg, 3.40 mmol) in DMSO (10 mL) was added DIEA (3.00 eq, 879 mg, 6.80 mmol) and then stirred for 20 min at 100° C. LCMS showed raw material was consumed completely and the major peak showed desired MS (M+H)⁺=590.2, purity=88.17%, UV=220 nm. Retention time=1.052 min. The reaction mixture was poured into water (50 mL) and then extracted with ethyl acetate (30 mL*2) and the organics was washed with saturated brine solution (10 mL). The organics were then separated and dried (Na₂SO₄) before concentration to dryness. The crude was then purified by silica gel column (PE/EA=1/1, Rf=0.5) to give (2S,6R)-2-(1-cyclopropylpyrazol-4-yl)-4-[4-(4′-fluorospiro[1,3-dithiolane-2,1′-indane]-5′-yl)-6,7-dimethyl-pteridin-2-yl]-6-methyl-morpholine (900 mg, 1.53 mmol, 67.30% yield) as red solid. ¹H NMR (400 MHz, CDCl3) δ ppm 1.01 (q, J=6.32 Hz, 2H) 1.07-1.16 (m, 2 H)_{1.33} (d, J=6.11 Hz, 3H)_{2.61} (s, 3H)_{2.72} (s, 3H)_{3.04}-3.11 (m, 3H)_{3.47}-3.52 (m, 2H)_{3.55}-3.61 (m, 3H) 3.75-3.88 (m, 1H) 4.60 (br d, J=10.76 Hz, 1H) 4.90-5.03 (m, 1H) 7.48 (d, J=7.58 Hz, 1 H) 7.52-7.61 (m, 3H). LCMS (M+H)⁺=590.2, purity=88.17%, UV=220 nm. Retention time=1.052 min.

Step 5: A solution of NIS (4.00 eq, 76 mg, 0.339 mmol) in DCM (1 mL) was cooled to −78° C. in two plastic round bottom flask. HF-Py (70%, 0.10 mL, 0.0848 mmol) was added to the mixture respectively and stirred for 30 min, then followed by a solution of (2S,6R)-2-(1-cyclopropylpyrazol-4-yl)-4-[4-(4′-fluorospiro[1,3-dithiolane-2,1′-indane]-5′-yl)-6,7-dimethyl-pteridin-2-yl]-6-methyl-morpholine (1.00 eq, 50 mg, 0.0848 mmol) in DCM (0.50 mL) were added dropwise. Upon completion of addition the reaction mixture was warmed to −50° C. and stirred for 3 h. LCMS showed the raw material was consumed and the major peak showed desired MS (M+H)⁺=563.2, and MS (M+H)⁺=662.1. The reaction was poured into the mixture ice water (5 mL) and NaHCO₃ (aq. 10 mL) and then extracted with ethyl acetate (5 mL*3) and the organics washed with 5 mL saturated brine solution. The organics were then separated and dried (Na₂SO₄) before concentration to dryness. The crude was then purified by prep-TLC (PE/EA=1/2, Rf=0.4) to give (2S,6R)-2-(1-cyclopropylpyrazol-4-yl)-4-[6,7-dimethyl-4-(1,1,4-trifluoroindan-5-yl)pteridin-2-yl]-6-methyl-morpholine (20 mg, 0.0373 mmol, 22.02% yield) and (2S,6R)-2-(1-cyclopropylpyrazol-4-yl)-4-[6,7-dimethyl-4-(1,1,4-trifluoro-2-iodo-indan-5-yl)pteridin-2-yl]-6-methyl-morpholine (35 mg, 0.0529 mmol, 31.21% yield) as red solid. MS (M+H)⁺=536.2 and 662.1, purity=27.67% and 71.84%.

Step 6: To a solution of (2S,6R)-2-(1-cyclopropylpyrazol-4-yl)-4-[6,7-dimethyl-4-(1,1,4-trifluoro-2-iodo-indan-5-yl)pteridin-2-yl]-6-methyl-morpholine (1.00 eq, 55 mg, 0.0529 mmol) in DCM (1 mL) was added DBU (1.50 eq, 12 mg, 0.0793 mmol) and then the mixture was stirred at 20° C. for 2 h. LCMS showed raw material was consumed completely and the major peak showed desired MS (M+H)⁺=534.1, purity=87.56%, UV=220 nm. Retention time=1.010 min. The reaction was added HCl (1 M, 1 mL) and then 5 mL water was added, and then extracted with ethyl acetate (2 mL*2) and the organics washed with 3 mL saturated brine solution. The organics were then separated and dried (Na₂SO₄) before concentration to dryness and give crude (2S,6R)-2-(1-cyclopropylpyrazol-4-yl)-4-[6,7-dimethyl-4-(1,1,4-trifluoroinden-5-yl)pteridin-2-yl]-6-methyl-morpholine (60 mg, 0.112 mmol, 212.65% yield) as yellow solid and used to next step without further purification. MS (M+H)⁺=534.1, purity=87.56%, UV=220 nm. Retention time=1.01 min.

Step 7: To a solution of (2S,6R)-2-(1-cyclopropylpyrazol-4-yl)-4-[6,7-dimethyl-4-(1,1,4-trifluoroinden-5-yl)pteridin-2-yl]-6-methyl-morpholine (1.00 eq, 45 mg, 0.0843 mmol) in MeCN (1 mL) was added K₃PO₄ (0.200 eq, 3.6 mg, 0.0169 mmol) and 2-nitrobenzenesulfonohydrazide (2.00 eq, 37 mg, 0.169 mmol) to the mixture and then the mixture was stirred at 20° C. for 16 h. LCMS showed raw material was consumed and the major peak showed desired MS (M+H)⁺=536.2, purity=86.22%, UV=220 nm. Retention time=1.003 min. The reaction was added HCl (aq. 1 M) (1 mL) and then poured into water (5 mL), then extracted with ethyl acetate (3 mL*2), the organics washed with 3 mL saturated brine solution. The organics were then separated and dried (Na₂SO₄) before concentration to dryness. The crude was then purified by prep-TLC (PE/EA=1/3, Rf=0.3) twice to give (2S,6R)-2-(1-cyclopropylpyrazol-4-yl)-4-[6,7-dimethyl-4-(1,1,4-trifluoroindan-5-yl)pteridin-2-yl]-6-methyl-morpholine (15 mg, 0.0280 mmol, 33.25% yield) as a light-yellow solid. MS (M+H)⁺=536.2, purity=100%, uv=220 nm. Retention time=1.008 min. ¹H NMR (400 MHz, CDCl3) δ ppm 0.98-1.05 (m, 2H) 1.09-1.15 (m, 2H) 1.33 (d, J=6.11 Hz, 3H) 2.60 (s, 3H) 2.66-2.79 (m, 5H) 2.85 (dd, J=13.20, 10.76 Hz, 1H) 3.03-3.12 (m, 1H) 3.13-3.20 (m, 2H) 3.58 (tt, J=7.23, 3.65 Hz, 1H) 3.77-3.89 (m, 1H) 4.61 (br d, J=8.93 Hz, 1 H) 4.85-5.23 (m, 2H) 7.49 (br d, J=7.82 Hz, 1H) 7.52-7.58 (m, 2H) 7.62-7.70 (m, 1H).

### Synthesis of I-1490

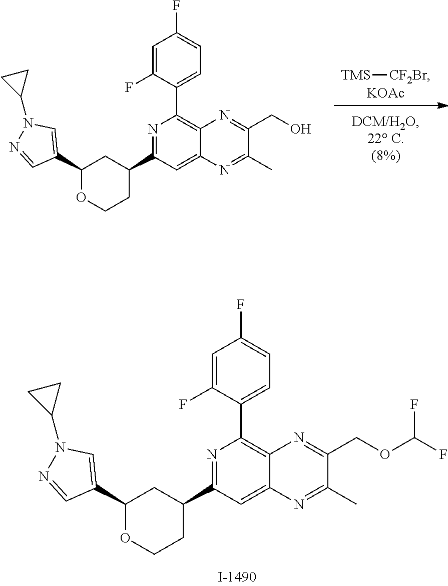

A glass vial equipped with a Teflon-coated magnetic stirring bar was charged with (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methanol (1.0 equiv, 119 mg, 0.25 mmol), potassium acetate (4.0 equiv, 98 mg, 1.0 mmol), DCM (0.15 mL) and water (0.15 mL). The resulting solution was cooled in an ice bath at 0° C. with stirring. [bromo(difluoro)methyl]-trimethyl-silane (2.0 equiv, 0.079 mL, 0.50 mmol) was added and the reaction mixture was stirred at 22° C. for 17 h. The reaction mixture was diluted with DCM and water. The layers were separated, and the aqueous layer was extracted with DCM (3×). The combined organic layers were dried over Na₂SO₄, filtered and evaporated to dryness under reduced pressure. The crude residual mixture was purified by silica gel flash column chromatography (Teledyne RediSep® GOLD column, 24 g SiO₂) using an elution gradient of 1% to 10% MeOH in DCM to afford 31 mg of the crude product. The product was further purified by preparative HPLC (Gemini® 5 um NX-C18 110 Å, 100×30 mm column) using an elution gradient of MeOH in 10 mM aqueous ammonium formate pH 3.8 (30-50%) to afford 7-((2R,4S)-2-(1-cyclopropyl-1H-pyrazol-4-yl)tetrahydro-2H-pyran-4-yl)-3-((difluoromethoxy)methyl)-5-(2,4-difluorophenyl)-2-methylpyrido[3,4-b]pyrazine (11 mg, 0.02 mmol, 8% yield) as an off-white solid. The product was a 3:1 mixture of cis:trans diastereomers. LC-MS(ESI⁺): Tr=0.94 min; [M+H]⁺ 528.3 (obs). ¹H NMR (DMSO-d₆, 400 MHz): ^{6H} 9.14-9.16 (4H, m), 9.05 (4H, s), 8.43 (9H, s), 7.97 (1H, s), 7.87 (3H, s), 7.63-7.70 (4H, m), 7.38 (4H, t, J=9.8 Hz), 7.24 (4H, t, J=8.5 Hz), 5.02 (1H, t, J=4.8 Hz), 4.65-4.69 (10H, m), 4.15 (3H, d, J=11.2 Hz), 4.01 (5H, s), 3.78 (5H, d, J=13.4 Hz), 2.77-2.78 (10H, m), 2.35 (4H, d, J=13.7 Hz), 1.85-1.98 (9H, m), 1.41-1.44 (9H, m), 1.19-1.24 (10H, m).

### Synthesis of 1505 (Same general method for I-1278)

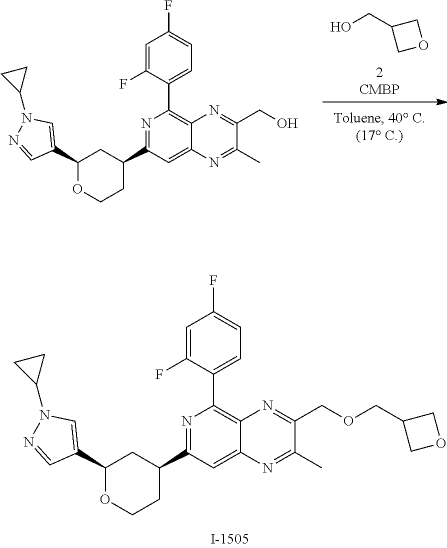

A flame-dried round-bottomed flask equipped with a Teflon-coated magnetic stirring bar was charged with (7-((2R,4S)-2-(1-cyclopropyl-1H-pyrazol-4-yl)tetrahydro-2H-pyran-4-yl)-5-(2,4-difluorophenyl)-2-methylpyrido[3,4-b]pyrazin-3-yl)methanol (1.0 equiv, 60 mg, 0.126 mmol). The flask was sealed and purged under Ar (g). Anhydrous toluene (1.5 mL) was added, followed by oxetan-3-ylmethanol (10.0 equiv, 0.10 mL, 1.26 mmol) and 2-(tributyl-,5-phosphanylidene)acetonitrile (3.00 equiv, 0.099 mL, 0.377 mmol). The resulting solution was stirred at 40° C. for 20 h. The reaction mixture cooled to 22° C., diluted with EtOAc and washed sequentially with sat. NH₄Cl (aq), sat. NaHCO₃ (aq) and brine. The organic layer was dried over Na₂SO₄, filtered and evaporated to dryness under reduced pressure. The crude residual mixture was purified by silica gel flash column chromatography (Teledyne RediSep® GOLD column, 24 g SiO₂) using an elution gradient of 0% to 10% MeOH in DCM to afford 296 mg of the crude product as a dark brown syrup. The product was further purified by preparative HPLC (Gemini® 5 um NX-C18 110 Å, 100×30 mm column) using an elution gradient of MeOH in 10 mM aqueous ammonium formate pH 3.8 (48-68%) to afford 7-((2R,4S)-2-(1-cyclopropyl-1H-pyrazol-4-yl)tetrahydro-2H-pyran-4-yl)-5-(2,4-difluorophenyl)-2-methyl-3-((oxetan-3-ylmethoxy)methyl)pyrido[3,4-b]pyrazine (12 mg, 0.021 mmol, 17% yield) as a white solid. LC-MS(ESI⁺): Tr=1.47 min; [M+H]⁺ 548.3 (obs). ¹H NMR (DMSO-d₆, 400 MHz): δ_{H} 7.88 (1H, s), 7.70-7.74 (2H, m), 7.39-7.43 (2H, m), 7.27 (1H, td, J=8.5, 2.6 Hz), 4.79 (2H, s), 4.55 (2H, dd, J=7.9, 5.9 Hz), 4.49 (1H, dd, J=10.8, 1.9 Hz), 4.23 (2H, t, J=6.0 Hz), 4.09 (1H, dd, J=11.2, 3.9 Hz), 3.63-3.72 (4H, m), 3.09-3.16 (1H, m), 2.76 (2H, s), 2.22 (1H, d, J=13.0 Hz), 1.87-1.96 (3H, m), 0.97-0.99 (2H, m), 0.88-0.93 (3H, m).

### Synthesis of Compounds I-1510 and I-1511

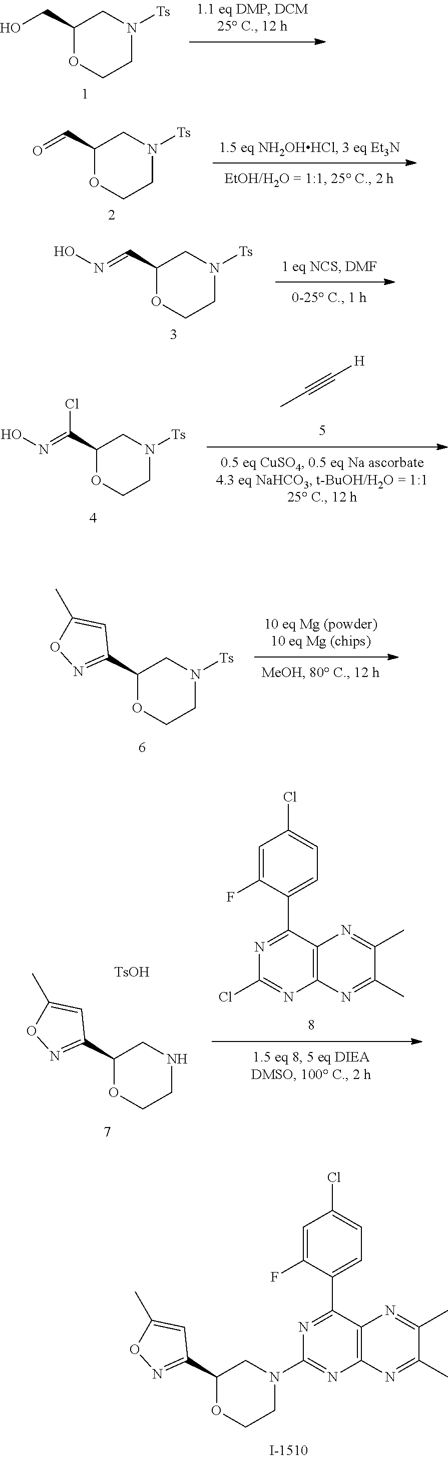

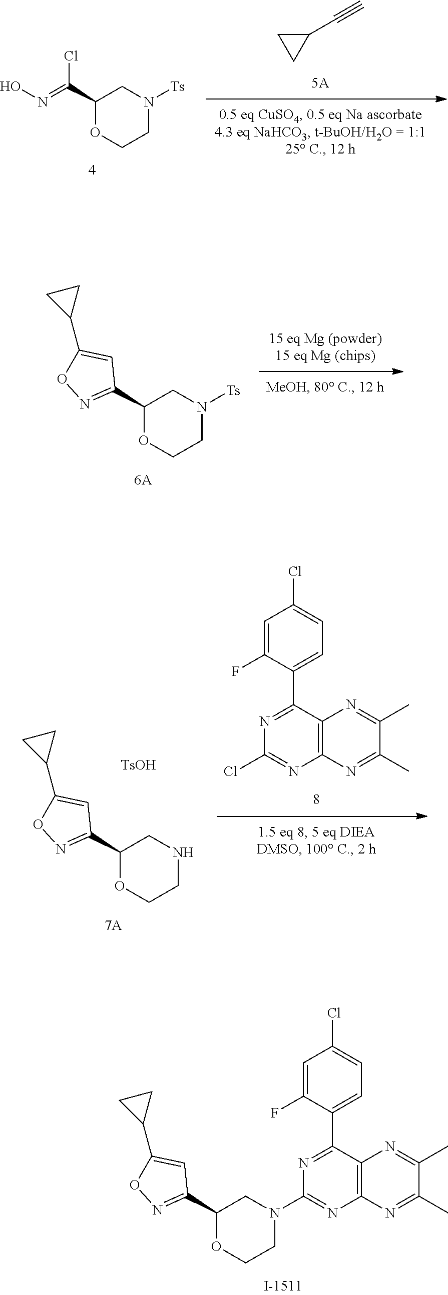

Step 1: To a solution of [(2R)-4-(p-tolylsulfonyl)morpholin-2-yl]methanol (1.00 eq, 1000 mg, 3.69 mmol) in DCM (20 mL) was added Dess-Martin periodinane (1.10 eq, 1720 mg, 4.05 mmol) at 0° C. Then the reaction solution was warmed to 25° C. and stirred for 16 hours. LCMS (5-95AB/1.5 min): RT=0.427 min, 270.0=[M+H]⁺, ESI⁺ showed 55% of desired product and RT=0.630 min, 272.1=[M+H]⁺, ESI⁺ showed 10% of starting material, after night, LCMS showed starting material was consumed completely. The reaction mixture was poured into saturated NH₄Cl aqueous solution (20 mL) and then extracted with ethyl acetate (20 mL*3). The combined organic layers were dried over Na₂SO₄, filtered and concentrated under reduced pressure to give a residue. The residue was purified by flash chromatography on silica gel eluting with [petroleum ether]/[ethyl acetate]=(100/1-3/1) (PE/EA=5:1, Rf=0.5) to afford (2R)-4-(p-tolylsulfonyl)morpholine-2-carbaldehyde (520 mg, 1.54 mmol, 41.91% yield) as a white solid, which LCMS [M+H]⁺=270.0; purity=84% (220 nm). Retention time=0.751 min.

Step 2: To a solution of NH₂OH·HCl (1.50 eq, 194 mg, 2.78 mmol) and (2R)-4-(p-tolylsulfonyl) morpholine-2-carbaldehyde (1.00 eq, 500 mg, 1.86 mmol) in Ethanol (4 mL) and Water (0.1000 mL), TEA (3.00 eq, 0.77 mL, 5.57 mmol) was added and stirred at 25° C. for 2 hours. LCMS (5-95AB/1.5 min): RT=0.786 min, 285.1=[M+H]⁺, ESI⁺ showed 71.5% of desired product. The reaction mixture was filtered. The filter cake was washed with water (2 mL*3). The filtrate was concentrated under reduced pressure to afford (2E,2R)-4-(p-tolylsulfonyl)morpholine-2-carbaldehyde oxime (285 mg, 0.952 mmol, 51.29% yield), as a white solid, which LCMS [M+H]⁺=285.1; purity=95% (220 nm). Retention time=0.494 min.

Step 3: A solution of (2E,2R)-4-(p-tolylsulfonyl)morpholine-2-carbaldehyde oxime (1.00 eq, 285 mg, 1.00 mmol) and NCS (1.00 eq, 178 mg, 1.00 mmol) in DMF (5 mL) was purged with N₂ for 3 times and stirred at 30° C. for 1 hour. It was used for the next step without further workup. [M+H]+=319.0; purity=90% (220 nm). Retention time=0.840 min.

Step 4: Both tert-butanol (2 mL) and water (2 mL) were added in reaction sealed tube, Na ascorbate (0.500 eq, 20 mg, 0.290 mmol), NaHCO₃ (4.30 eq, 210 mg, 2.50 mmol) and CuSO₄·H₂O (0.0500 eq, 7.2 mg, 0.0290 mmol) was added, the mixture was turned into brown at that time, then added prop-1-yne (1.00 eq, 0.58 mL, 0.580 mmol) slowly at N₂ atmosphere, finally, (2Z,2R)—N-hydroxy-4-(p-tolylsulfonyl)morpholine-2-carboximidoyl chloride (1.00 eq, 185 mg, 0.580 mmol) was added slowly in 0° C., during the time, the color changed from brown to yellow, light-yellow, colorless and light bule, eventually stirred at 30° C. for 2 hours. LCMS (5-95AB/1.5 min): RT=0.873 min, 323.1=[M+H]⁺, ESI⁺ showed 22.5% of desired product and no starting material. The reaction mixture was extracted with ethyl acetate (20 mL*3). The combined organic layers were dried over Na₂SO₄, filtered and concentrated under reduced pressure to give a residue. The residue was purified by prep-HPLC (Column, [Phenomenex luna C18 250*50 mm*10 um]; mobile phase: [ACN] and [H2O] (conditions: [water (0.225% FA)-ACN], B %: 65%-90%; Detector, UV 254 nm. RT: [22 min]) and lyophilized to afford 2-(5-methylisoxazol-3-yl)-4-(p-tolylsulfonyl) morpholine (20 mg, 0.0620 mmol, 10.69% yield) as a white solid, which LCMS. [M+H]⁺=323.1; purity=100% (220 nm). Retention time=0.876 min.

Step 5: To a solution of (2R)-2-(5-methylisoxazol-3-yl)-4-(p-tolylsulfonyl)morpholine (1.00 eq, 20 mg, 0.0620 mmol) in Methanol (2 mL) was added Mg (powder) (15.0 eq, 22 mg, 0.931 mmol) and Mg (chips) (15.0 eq, 22 mg, 0.931 mmol) at 25° C. and then purged with N₂ for 3 times and stirred for 12 hours at 80° C. LCMS (5-95AB/1.5 min) showed 28% of starting material. Then the mixture in Methanol (6 mL) was added Mg (powder) (20.0 eq, 30 mg, 1.24 mmol) and Mg (chips) (20.0 eq, 30 mg, 1.24 mmol) at 25° C. and then purged with N₂ for 3 times and stirred for 12 hours at 80° C. The reaction mixture was filtered through celatom, the filtrate was evaporated under reduced pressure to give 4-methylbenzenesulfonic acid; (2R)-2-(5-methylisoxazol-3-yl) morpholine (30 mg, 142.06% yield) as crude product. [M+H]⁺=323.1; purity=28% (220 nm). Retention time=0.883 min.

Step 6: To a solution of 4-methylbenzenesulfonic acid; (2R)-2-(5-methylisoxazol-3-yl)morpholine (1.20 eq, 29 mg, 0.0856 mmol) and 2-chloro-4-(4-chloro-2-fluoro-phenyl)-6,7-dimethyl-pteridine (1.00 eq, 23 mg, 0.0713 mmol) in DMSO (0.5000 mL) was added DIEA (4.17 eq, 38 mg, 0.297 mmol)) at 100° C. stirred for 2 hours. LCMS (5-95AB/1.5 min): RT=0.991 min, 455.1=[M+H]⁺, ESI⁺ showed 33.5% of desired product. The reaction mixture was poured into H₂O (20 mL) and extracted with ethyl acetate (20 mL*3). The combined organic layers were dried over Na₂SO₄, filtered and concentrated under reduced pressure to give a residue. The residue was purified by prep-HPLC (Phenomenex Synergi C18 150*25 mm*10 um), Condition: water(FA)-ACN, Gradient Time(min): 10) and lyophilized to afford (2R)-4-[4-(4-chloro-2-fluoro-phenyl)-6,7-dimethyl-pteridin-2-yl]-2-(5-methylisoxazol-3-yl)morpholine (2.4 mg, 0.00512 mmol, 7.18% yield) as yellow solid. [M+H]⁺=455.1; purity=33.5% (220 nm). Retention time=0.991 min. 1H NMR (400 MHz, CDCl₃) δ=7.66 (t, J=7.8 Hz, 1H), 7.31 (br d, J=8.2 Hz, 1H), 7.25 (s, 1H), 6.11 (s, 1H), 5.16 (br d, J=12.7 Hz, 1H), 4.90 (br d, J=13.8 Hz, 1H), 4.78-4.71 (m, 1H), 4.15 (br d, J=9.7 Hz, 1H), 3.89-3.80 (m, 1H), 3.44-3.30 (m, 2H), 2.72 (s, 3H), 2.60 (s, 3H), 2.45 (s, 3H).

Step 7: Both tert-butanol (2.5 mL) and Water (2.5 mL) were added in sealed tube, Na ascorbate (0.500 eq, 21 mg, 0.308 mmol), NaHCO₃ (4.30 eq, 223 mg, 2.65 mmol) and CuSO₄·H₂O (0.0500 eq, 7.7 mg, 0.0308 mmol) was added, the reaction mixture turned into brown, then ethynylcyclopropane (1.00 eq, 41 mg, 0.617 mmol) was slowly added at N₂ atmosphere, finally, (2Z,2R)—N-hydroxy-4-(p-tolylsulfonyl)morpholine-2-carboximidoyl chloride (1.00 eq, 197 mg, 0.617 mmol) was added at 0° C., during the time, the reaction color changed from brown to yellow, light-yellow, colorless and light bule, eventually stirred at 30° C. for 2 hours. LCMS (5-95AB/1.5 min): RT=0.912 min, 349.1=[M+H]⁺, ESI⁺ showed 60% of desired product. The reaction mixture was extracted with ethyl acetate (20 mL*3). The combined organic layers were dried over Na₂SO₄, filtered and concentrated under reduced pressure to afford (2R)-2-(5-cyclopropylisoxazol-3-yl)-4-(p-tolylsulfonyl)morpholine (25 mg, 0.0718 mmol, 11.63% yield) as a white solid. [M+H]⁺=349.1; purity=60% (220 nm). Retention time=0.912 min.

Step 8: a solution of (2R)-2-(5-cyclopropylisoxazol-3-yl)-4-(p-tolylsulfonyl)morpholine (1.00 eq, 25 mg, 0.0718 mmol) in Methanol (6 mL) was added Mg(powder) (15.0 eq, 26 mg, 1.08 mmol) and Mg(chips) (15.0 eq, 26 mg, 1.08 mmol) at 25° C. and then purged with N₂ for 3 times and stirred for 12 hours at 80° C. LCMS (5-95AB/1.5 min): RT=0.206 min, 195.3=[M+H]⁺, ESI⁺ showed 43% of desired product and RT=0.932 min, 349.2=[M+H]⁺, ESI⁺ showed 48.6% of starting material. Then Mg(powder) (15.0 eq, 26 mg, 1.08 mmol) and Mg(filings) (15.0 eq, 26 mg, 1.08 mmol) was added at 25° C. and then purged with N₂ for 3 times and stirred for 12 hour at 80° C. The reaction mixture was filtered through celatom, the filtrate was evaporated under reduced pressure to give 4-methylbenzenesulfonic acid; (2R)-2-(5-cyclopropylisoxazol-3-yl) morpholine (50 mg, 0.136 mmol, 190.17% yield) as white solid. [M+H]⁺=195.3; purity=43% (220 nm). Retention time=0.206 min.

Step 9: A solution of 4-methylbenzenesulfonic acid; (2R)-2-(5-cyclopropylisoxazol-3-yl)morpholine (2.21 eq, 50 mg, 0.136 mmol) and 2-chloro-4-(4-chloro-2-fluoro-phenyl)-6,7-dimethyl-pteridine (1.00 eq, 20 mg, 0.0618 mmol) in DMSO (1 mL) was added DIEA (4.17 eq, 33 mg, 0.257 mmol) and stirred at 100° C. for 2 hours. LCMS (5-95AB/1.5 min): RT=1.030 min, 481.1=[M+H]⁺, ESI⁺ showed 44% of desired product. The reaction mixture was poured into H₂O (20 mL) and extracted with ethyl acetate (20 mL*3). The combined organic layers were dried over Na₂SO₄, filtered and concentrated under reduced pressure to give a residue. The residue was purified by prep-HPLC (Phenomenex Synergi C18 150*25 mm*10 um), Condition: water(FA)-ACN, Gradient Time(min): 10) and lyophilized to afford (2R)-4-[4-(4-chloro-2-fluoro-phenyl)-6,7-dimethyl-pteridin-2-yl]-2-(5-cyclopropylisoxazol-3-yl)morpholine (2.3 mg, 0.00462 mmol, 7.48% yield) as yellow solid. LCMS (M+H)⁺=481.1; purity=44% (220 nm). Retention time=1.030 min. 1H NMR (400 MHz, CDCl₃) δ=7.66 (t, J=7.8 Hz, 1H), 7.31 (br d, J=8.6 Hz, 1H), 7.25 (d, J=1.6 Hz, 1H), 6.01 (s, 1H), 5.21-5.10 (m, 1H), 4.89 (br d, J=13.6 Hz, 1H), 4.73 (dd, J=2.6, 10.3 Hz, 1H), 4.15 (br dd, J=1.5, 11.4 Hz, 1H), 3.89-3.78 (m, 1H), 3.42-3.30 (m, 2H), 2.72 (s, 3H), 2.60 (s, 3H), 2.10-2.00 (m, 1H), 1.12-1.04 (m, 2H), 1.00-0.94 (m, 2H).

### Synthesis of I-1513

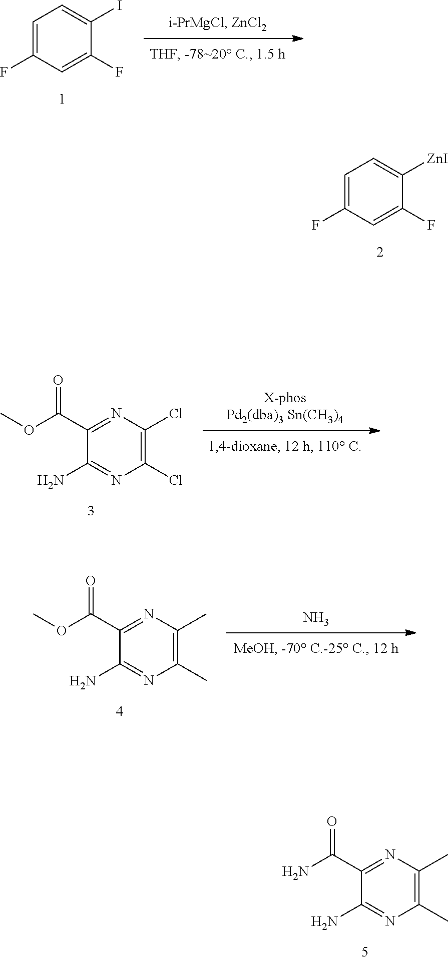

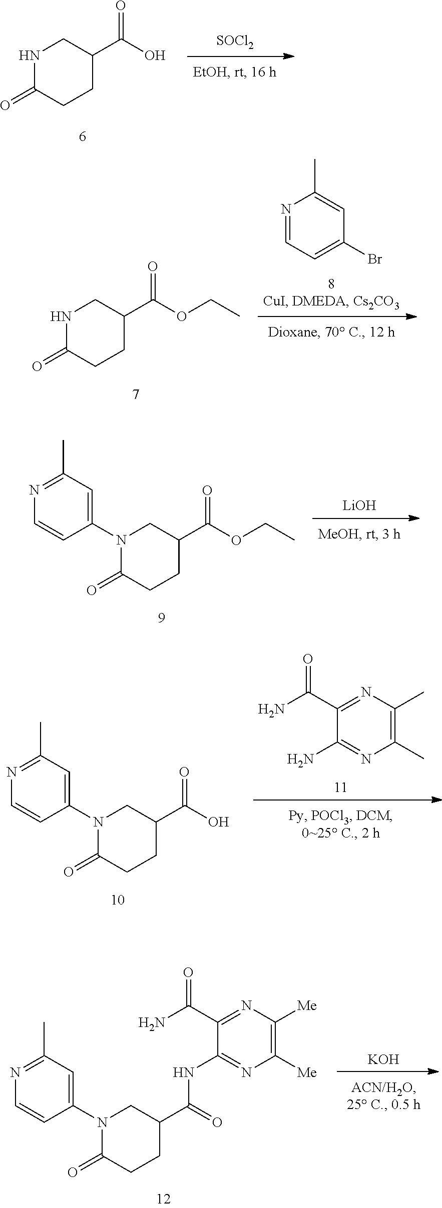

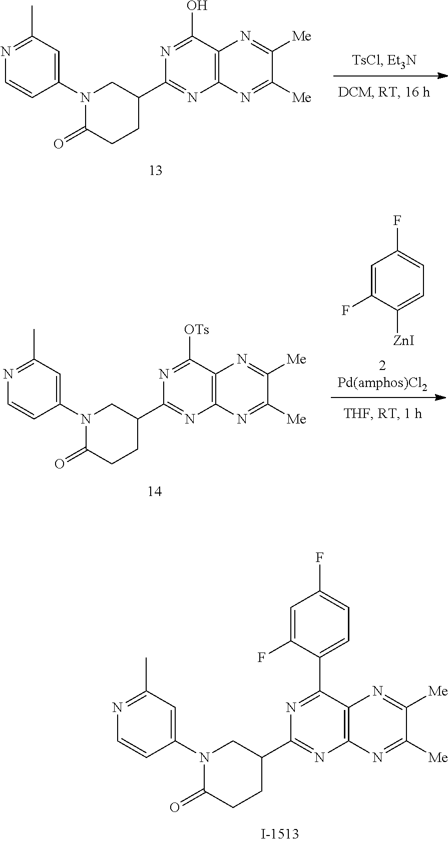

Step 1: To a mixture of methyl 3-amino-5,6-dichloro-pyrazine-2-carboxylate (4 g, 18.0 mmol, 1.0 eq) and tetramethylstannane (8.05 g, 45.0 mmol, 2.5 eq) in 1,4-dioxane (50 mL) were added X-phos (3.43 g, 7.2 mmol, 0.4 eq) and Pd₂(dba)₃ (1.03 g, 1.8 mmol, 0.1 eq). The mixture was degassed and stirred under N₂, then warmed up to 110° C. and stirred overnight. LCMS showed the main peak was the desired product and no starting material remained. The mixture was cooled to room temperature, diluted with water (50 mL), and extracted with CH₂Cl₂ (100 mL×3). The combined organics were washed with NaHCO₃ (aq) (100 mL) and brine (100 mL), dried over Na₂SO₄, and concentrated in vacuum to afford the crude product. The crude product was purified by FCC (Petroleum ether:Ethyl acetate=52:48) to afford the desired product (3 g, 87.3%) as a yellow solid. LCMS: M+H=182, Ref time=0.85.

Step 2: In a sealed tube, methyl 3-amino-5,6-dimethyl-pyrazine-2-carboxylate (300 mg, 1.66 mmol) was added to a solution of NH₃ in MeOH (2 mL, 7 mol/L). The mixture was warmed up to 80° C. and stirred overnight. The reaction was concentrated, and the residue was washed with MTBE (50 mL). The solid was dried in vacuum to afford the desired product (295 mg, 96.5%). LCMS: M+H=167.2, Ref time=0.572 min

Step 3: To a mixture of 6-oxopiperidine-3-carboxylic acid (5 g, 34.9 mmol, 1.0 eq) in EtOH (50 mL) was added thionyl chloride (52.4 mmol, 3.8 mL, 1.5 eq) dropwise. The mixture was stirred at room temperature overnight. The mixture was concentrated in vacuum to afford the crude product. After washed with MTBE (100 mL), the solid was collected. The solid was dried in vacuum to afford the desired product (7 g, 100%). LCMS: M+H=172.2, Ref time=0.514. 1H NMR (400 MHz, DMSO) δ 10.28 (s, 1H), 4.13-4.07 (m, 2H), 3.34-3.23 (m, 2H), 2.83-2.77 (m, 1H), 2.27-2.12 (m, 2H), 2.02-1.95 (m, 1H), 1.88-1.79 (m, 1H), 1.21-1.17 (t, J=6.8 Hz, 3H).

Step 4: To a mixture of ethyl 6-oxopiperidine-3-carboxylate (1.4 g, 8.18 mmol, 95% purity, 1.0 eq), 4-bromo-2-methylpyridine (1.40 g, 8.18 mmol, 1.0 eq) and DMEDA (793 mg, 9 mmol, 1.1 eq) CuI (779 mg, 4.09 mmol, 0.5 eq) in 1,4-dioxane (30 mL) was added Cs₂CO₃ (5.32 g, 16.36 mmol, 2.0 eq) The mixture was degassed 3 times and stirred under N₂. The mixture was warmed up to 110° C. overnight. The mixture was filtered and the organic collected, dried with Na₂SO₄, concentrated in vacuum to give the crude product. The crude product was purified by FCC (DCM:MeOH=10:1) to give the desired product (1.4 g, 65.3%) as a yellow oil. M+H=263.1 Ref time=0.546 min.

Step 5: To a solution of ethyl 1-(2-methyl-4-pyridyl)-6-oxo-piperidine-3-carboxylate (2.5 g, 9.53 mmol, 1.0 eq) in MeOH (40 mL) was added a solution of LiOH (229 mg, 9.53 mmol, 1.0 eq) in H₂O (40 mL). After stirring 30 mins, LCMS showed the starting material was consumed. The reaction was adjusted pH=4 with 1 M HCl and freeze-dried to get the product (1.5 g, 67.2%). LCMS: M+H=235.1 Ref time=0.246.

Step 6: A mixture of 1-(2-methyl-4-pyridyl)-6-oxo-piperidine-3-carboxylic acid (800 mg, 3.42 mmol, 1.0 eq) in DCM (15 mL) was added 3-amino-5,6-dimethyl-pyrazine-2-carboxamide (567 mg, 3.45 mmol, 1.0 eq) and pyridine (1.62 g, 20.5 mmol, 6.0 eq), the mixture was stirred and cooled down to 0° C., then POCl₃ (944 mg, 6.83 mmol, 2.0 eq) in DCM (5 mL) was added dropwise. After completion, the mixture was warmed up to 25° C., and stirred for 3 hours. The mixture was concentrated in vacuum and used for next step. LCMS: M+H=383.4 Ref time=1.057.

Step 7: To a mixture of 5,6-dimethyl-3-[[1-(2-methyl-4-pyridyl)-6-oxo-piperidine-3-carbonyl]amino]pyrazine-2-carboxamide (1.5 g, 3.9 mmol, 1.0 eq) in MeCN (20 mL) was added a solution of KOH (2.2 g, 39 mmol, 10 eq) in water (20 mL). The mixture was stirred at room temperature for 1 hour. The mixture was freeze-dried and was purified by FCC (CH₂Cl₂: MeOH=100-90%) to afford the desired product (300 mg).

Step 8: To a solution of 5-(4-hydroxy-6,7-dimethyl-pteridin-2-yl)-1-(2-methyl-4-pyridyl)piperidin-2-one (260 mg, 0.71 mmol, 1.00 eq) and TsCl (204 mg, 1.07 mmol, 1.5 eq) in DCM and MeCN (15+15 mL) was added Et₃N (216 mg, 2.14 mmol, 3 eq). The reaction mixture was stirred at 25° C. under N₂ for 5 hours. LC-MS showed starting material was consumed and one main peak with desired m/z was detected. The mixture was evaporated and purified by FCC (CH₂Cl₂: MeOH=100-95%) to afford the desired product. LCMS: M+H=519.5 Ref time=1.49.

Step 9: To a solution of 2,4-difluoro-1-iodo-benzene (300 mg, 1.25 mmol, 1.00 eq) in THF (5 mL) was added i-PrMgCl (0.65 mL, 2.00 M, 1.1 eq) dropwise at −40° C. The mixture was stirred at the same temperature for 30 minutes. Then the reaction mixture was cooled to −78° C., and ZnCl₂ (180 mg in 2 mL THF, 2.00 M, 1.05 eq) was added dropwise and the reaction mixture was allowed to warm to 20° C. for 1 hour, and a white turbid liquid formed. The crude product was used directly for next reaction.

Step 10: To a mixture of [6,7-dimethyl-2-[1-(2-methyl-4-pyridyl)-6-oxo-3-piperidyl]pteridin-4-yl] 4-methylbenzenesulfonate (300 mg, 0.58 mmol, 1 eq) and Pd(amphos)Cl₂ (24 mg, 0.03 mmol, 0.05 eq) in THF (5 mL) was added a solution of (2,4-difluorophenyl)-iodo-zinc in THF (5 mL). The mixture was stirred at room temperature for 3 hours under N₂. The mixture was filtered and evaporated and purified by prep-HPLC (ACN-H₂O (0.1% NH₃); gradient: 5-95) and lyophilized to afford the desired product as a yellow solid (48.14 mg, 20% yield). LCMS: (M+H)⁺=461.5. Retention time=1.492 min. HPLC: purity=98.3% (254 nm); purity=98.9% (214 nm). Retention time=2.370 min. ¹H NMR (400 MHz, DMSO) δ 8.39 (d, J=5.5 Hz, 1H), 7.82-7.73 (m, 1H), 7.53-7.45 (m, 1H), 7.36-7.29 (m, 2H), 7.27-7.22 (m, 1H), 4.33-4.28 (m, 1H), 4.15-4.11 (m, 1H), 3.89-3.80 (m, 1H), 2.79 (s, 3H), 2.7-2.70 (m, 1H), 2.67 (s, 3H), 2.60-2.55 (m, 1H), 2.44 (s, 3H), 2.41-2.38 (m, 2H).

### Synthesis of Compounds I-1529 and I-1532

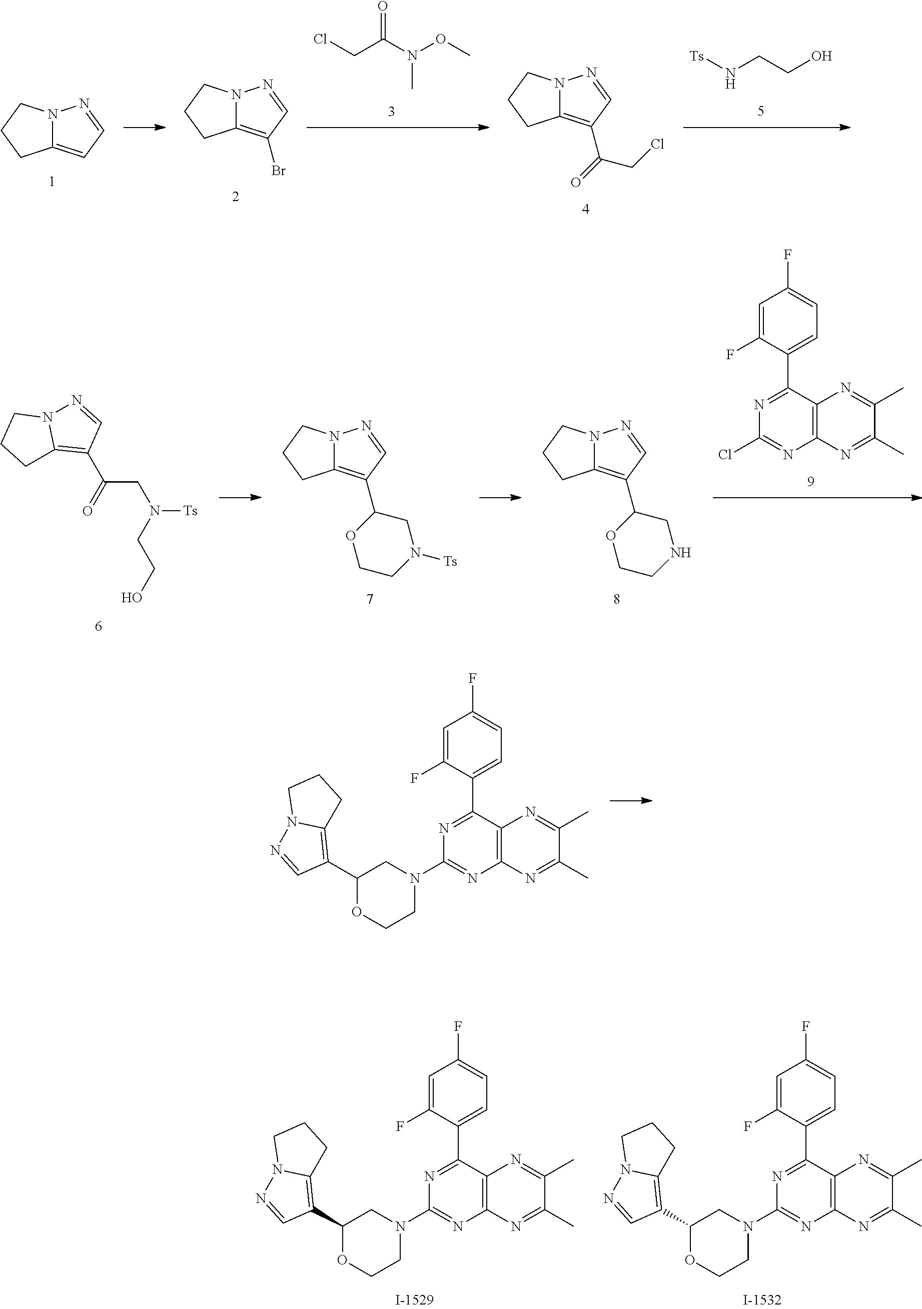

Step 1: To a solution of 5,6-dihydro-4H-pyrrolo[1,2-b]pyrazole (1.00 eq, 500 mg, 4.62 mmol) in DMF (10 mL) was added NBS (1.00 eq, 823 mg, 4.62 mmol) and then stirred for 16 h at 20° C. LCMS showed raw material was consumed and the major peak showed desired MS (187.0 [M+H]⁺, LC-RT=0.698 min). TLC (PE/EA=1/1) showed raw material was consumed and the new spot formed. The reaction was added water (30 mL) and then extracted with ethyl acetate (10 mL*3) and the organics washed with 10 mL saturated brine solution. The organics were then separated and dried (Na₂SO₄) before concentration to dryness. The crude was then purified by silica gel column (PE/EA=1/1) to give 3-bromo-5,6-dihydro-4H-pyrrolo[1,2-b]pyrazole (500 mg, 2.67 mmol, 57.82% yield) as white solid. MS (M+H)⁺=187.0; purity=100% (220 nm). Retention time=0.698 min. 1H NMR (400 MHz, CDCl3) δ ppm 2.58-2.68 (m, 2H) 2.82-2.93 (m, 2H) 4.14-4.24 (m, 2H) 7.42-7.46 (m, 1H).

Step 2. To a solution of 3-bromo-5,6-dihydro-4H-pyrrolo[1,2-b]pyrazole (1.00 eq, 350 mg, 1.87 mmol) in THF (10 mL) was added i-PrMgCl LiCl (1.50 eq, 2.2 mL, 2.81 mmol) at 0° C. and stirred for 5 h at 15° C., 2-chloro-N-methoxy-N-methylacetamide (1.10 eq, 283 mg, 2.06 mmol) was added to the mixture at 0° C. and then stirred for 10 min at 15° C. LCMS showed raw material was consumed most and the major peak of 74.6% showed desired MS (185.1 [M+H]⁺. Retention time=0.278 min). The reaction was added NH₄Cl (aq. 10 mL) and water (20 mL) then the solution extracted with ethyl acetate (5 mL*3) and the organics washed with 10 mL saturated brine solution. The organics were then separated and dried (Na₂SO₄) before concentration to dryness. The crude was then purified by silica gel column (PE/EA=0/1, Rf=0.3) to give 2-chloro-1-(5,6-dihydro-4H-pyrrolo[1,2-b]pyrazol-3-yl)ethanone (160 mg, 0.867 mmol, 46.31% yield) as white solid. MS (M+H)⁺=185.1; purity=74.6% (220 nm). Retention time=0.278 min. ¹H NMR (400 MHz, CDCl3) δ ppm 2.66-2.76 (m, 2H) 3.11-3.20 (m, 2H) 4.17-4.25 (m, 2H) 4.37-4.41 (m, 2H) 7.93-8.00 (m, 1H).

Step 3: To a solution of 2-chloro-1-(5,6-dihydro-4H-pyrrolo[1,2-b]pyrazol-3-yl)ethanone (1.00 eq, 160 mg, 0.867 mmol) in acetone (5 mL) was added N-(2-hydroxyethyl)-4-methyl-benzenesulfonamide (1.20 eq, 224 mg, 1.04 mmol), K₂CO₃ (3.00 eq, 359 mg, 2.60 mmol) and KI (1.00 eq, 144 mg, 0.867 mmol) and stirred for 16 h at 15° C. LCMS showed raw material was consumed completely and the major peak showed desired MS (M+H)⁺=364.1. Retention time=0.795 min. The reaction was poured into water (20 mL) and then extracted with ethyl acetate (10 mL*2) and the organics washed with 10 mL saturated brine solution. The organics were then separated and dried (Na₂SO₄) before concentration to dryness. The crude was then purified by silica gel column (PE/EA=1/0 1/1) to give N-[2-(5,6-dihydro-4H-pyrrolo[1,2-b]pyrazol-3-yl)-2-oxo-ethyl]-N-(2-hydroxyethyl)-4-methyl-benzenesulfonamide (150 mg, 0.413 mmol, 47.62% yield) as white solid. MS (M+H)⁺=364.1; purity=99.759% (220 nm). Retention time=0.795 min. ¹H NMR (400 MHz, CDCl3) δ ppm 2.44 (s, 2H) 2.41-2.41 (m, 1H) 2.64-2.77 (m, 2H) 3.16 (br t, J=7.38 Hz, 2H) 3.37 (br d, J=3.25 Hz, 2H) 3.58-3.69 (m, 3H) 4.13-4.26 (m, 2H) 4.41-4.50 (m, 2H) 7.30-7.35 (m, 2H) 7.73-7.79 (m, 2H) 7.93-7.97 (m, 1H).

Step 4: To a solution of N-[2-(5,6-dihydro-4H-pyrrolo[1,2-b]pyrazol-3-yl)-2-oxo-ethyl]-N-(2-hydroxyethyl)-4-methyl-benzenesulfonamide (1.00 eq, 150 mg, 0.413 mmol) in DCM (5 mL) was added TES (5.00 eq, 473 mg, 2.06 mmol) and TMSOTf (5.00 eq, 0.37 mL, 2.06 mmol) at 0° C. and then stirred for 16 h at 15° C. LCMS showed raw material consumed and the major peak showed desired MS (M+H)⁺=348.1. Retention time=0.866 min. The reaction was poured into water (20 mL) and then extracted with DCM (10 mL*2) and then organics washed with 10 mL saturated brine solution. The organics were then separated and dried (Na₂SO₄) before concentration to dryness. The crude was then purified by silica gel column (PE/EA=1/1, Rf=0.5) to give 2-(5,6-dihydro-4H-pyrrolo[1,2-b]pyrazol-3-yl)-4-(p-tolylsulfonyl)morpholine (95 mg, 0.273 mmol, 66.25% yield) as white solid. MS (M+H)⁺=348.1; purity=100% (220 nm). Retention time=0.866 min. 1H NMR (400 MHz, CDCl3) δ ppm 2.03-2.08 (m, 1H) 2.37-2.43 (m, 1H) 2.45-2.47 (m, 3H) 2.48-2.55 (m, 1H) 2.55-2.63 (m, 2H) 2.77-2.93 (m, 2H)_{3.50}-3.59 (m, 1H)_{3.62}-3.71 (m, 1H)_{3.73}-3.85 (m, 1H)_{3.93}-4.02 (m, 1H)_{4.06}-4.17 (m, 3H) 4.51-4.59 (m, 1H) 7.33-7.39 (m, 2H) 7.40-7.44 (m, 1H) 7.62-7.68 (m, 2H).

Step 5: To a solution of 2-(5,6-dihydro-4H-pyrrolo[1,2-b]pyrazol-3-yl)-4-(p-tolylsulfonyl)morpholine (1.00 eq, 95 mg, 0.273 mmol) in Methanol (5 mL) was added Mg powder (12.2 eq, 80 mg, 3.33 mmol) and Mg chips (12.2 eq, 80 mg, 3.33 mmol) at 25° C. and then stirred for 16 h at 80° C. LCMS showed 17% of raw material remained and the major peak showed desired MS (M+H)⁺=194.1. The reaction was cooled to 20° C. and Mg chips (12.2 eq, 80 mg, 3.33 mmol) was added to the solution and stirred for 16 h at 80° C. LCMS showed the major peak showed 91.8% desired MS (M+H)⁺=194.1. The reaction solution was filter and the filtrate was concentrated in vacuo to give 2-(5,6-dihydro-4H-pyrrolo[1,2-b]pyrazol-3-yl)morpholine (100 mg, 0.259 mmol, 94.62% yield) as white solid. MS (M+H)⁺=194.1; purity=91.8% (220 nm). Retention time=0.275 min.

Step 6: To a solution of 2-chloro-4-(2,4-difluorophenyl)-6,7-dimethyl-pteridine (1.00 eq, 100 mg, 0.326 mmol) and 2-(5,6-dihydro-4H-pyrrolo[1,2-b]pyrazol-3-yl)morpholine (1.50 eq, 95 mg, 0.489 mmol) in dry DMSO (1 mL) and DIEA (3.00 eq, 126 mg, 0.978 mmol) was added, then stirred for 20 min at 100° C. LCMS showed raw material was consumed completely and the major peak showed desired MS (M+H)⁺=464.2, purity=54.7% (220 nm). Retention time=0.915 min. The mixture was poured into water (10 mL) and then extracted with ethyl acetate (5 mL*2) and the organics washed with 5 mL saturated brine solution. The organics were then separated and dried (Na₂SO₄) before concentration to dryness. The crude was then purified by prep-HPLC (Unisil 3-100 C18 Ultra 150*50 mm*3 um, water(FA)-ACN) and freeze-drying to give 4-[4-(2,4-difluorophenyl)-6,7-dimethyl-pteridin-2-yl]-2-(5,6-dihydro-4H-pyrrolo[1,2-b]pyrazol-3-yl)morpholine (25 mg, 0.0547 mmol, 16.79% yield) as yellow solid (racemate): MS (M+H)⁺=464.1; purity=100% (220 nm). Retention time=0.922 min. ¹H NMR (400 MHz, CDCl3) δ ppm 2.59-2.68 (m, 5H) 2.73 (s, 3H) 2.92-3.06 (m, 2H) 3.27-3.43 (m, 2H) 3.82 (td, J=11.54, 2.69 Hz, 1H) 4.03-4.22 (m, 3H) 4.56 (dd, J=10.44, 2.69 Hz, 1H) 4.88 (br d, J=13.51 Hz, 1H) 5.03 (br d, J=11.13 Hz, 1H) 6.93-7.11 (m, 2H) 7.58 (s, 1H) 7.67-7.80 (m, 1H).

Step 7: The product mixture from step 6 was purified by SFC (DAICEL CHIRALCEL OJ (250 mm*30 mm, 10 um) 0.1% NH₃H₂O in MeOH) to give (2R)-4-[4-(2,4-difluorophenyl)-6,7-dimethyl-pteridin-2-yl]-2-(5,6-dihydro-4H-pyrrolo[1,2-b]pyrazol-3-yl)morpholine (11.21 mg, 0.0233 mmol, 46% yield) as yellow solid and (2S)-4-[4-(2,4-difluorophenyl)-6,7-dimethyl-pteridin-2-yl]-2-(5,6-dihydro-4H-pyrrolo[1,2-b]pyrazol-3-yl)morpholine (10.52 mg, 0.0221 mmol, 43.90% yield) as yellow solid. MS (M+H)⁺=464.1; purity=97.197% (220 nm). Retention time=0.914 min. 1H NMR (400 MHz, CDCl3) δ ppm 2.57-2.66 (m, 5H) 2.70-2.74 (m, 3H) 2.92-3.02 (m, 2H) 3.24-3.39 (m, 2H) 3.80 (td, J=11.55, 2.69 Hz, 1H) 4.04-4.17 (m, 3H) 4.54 (dd, J=10.39, 2.69 Hz, 1H) 4.86 (br d, J=13.33 Hz, 1H) 5.01 (br d, J=12.84 Hz, 1H) 6.92-7.09 (m, 2H) 7.53-7.58 (m, 1H) 7.66-7.77 (m, 1H). MS (M+H)⁺=464.2; purity=96.447% (220 nm). Retention time=0.919 min. 1H NMR (400 MHz, CDCl3) δ ppm 2.57-2.66 (m, 5H) 2.69 (br s, 3 H) 2.91-3.01 (m, 2H) 3.23-3.41 (m, 2H) 3.80 (td, J=11.43, 2.08 Hz, 1H) 4.03-4.19 (m, 3H) 4.54 (dd, J=10.21, 2.26 Hz, 1H) 4.74-4.90 (m, 1H) 4.92-5.09 (m, 1H) 6.91-7.11 (m, 2H) 7.45-7.60 (m, 1H) 7.65-7.79 (m, 1H).

### Synthesis of Compound I-1538

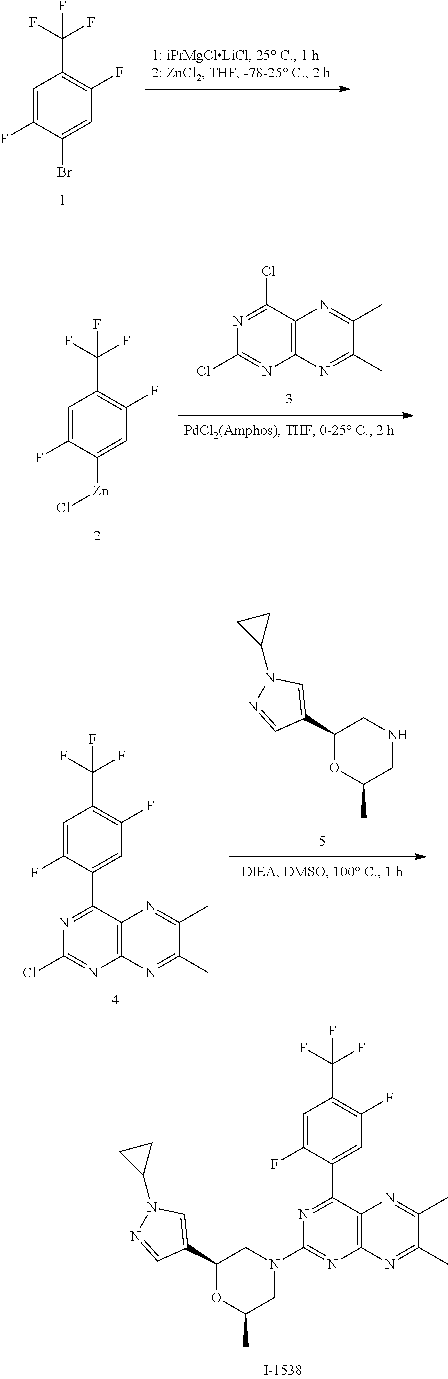

Step 1: To a solution of 1-bromo-2,5-difluoro-4-(trifluoromethyl)benzene (1.00 eq, 108 mg, 0.414 mmol) in THF (3 mL) was purged with N₂ for 3 times, iPrMgCl—LiCl (1.3 M in THF) (1.10 eq, 0.35 mL, 0.455 mmol) was added slowly at 25° C. and stirred for 1 h, then the reaction mixture was cooled to −78° C. and zinc chloride (0.5M in THF) (1.21 eq, 1.0 mL, 0.501 mmol) was added dropwise. The reaction mixture was allowed to 25° C. and stirred for 2 h. The reaction solution was used directly for next step.

Step 2: A sealed bottle under N₂ atmosphere was charged with chloro-[2,5-difluoro-4-(trifluoromethyl)phenyl]zinc (1.60 eq, 197 mg, 0.698 mmol) and PdCl₂(Amphos) (0.0500 eq, 15 mg, 0.0218 mmol) and THF (2 mL) and purged with N₂ three times, then cooled to 0° C., 2,4-dichloro-6,7-dimethyl-pteridine (1.00 eq, 100 mg, 0.437 mmol) was added dropwise to the reaction solution at 0° C., then warmed to 25° C. and stirred for 1 h. LCMS showed the reactant was consumed and 25% of desired mass was detected, the reaction solution was poured into H₂O, extracted with EtOAc and dried in vacuo to give the residue, which was then purified with Prep-HPLC(FA) and lyophilized to give 2-chloro-4-[2,5-difluoro-4-(trifluoromethyl)phenyl]-6,7-dimethyl-pteridine (10 mg, 0.0267 mmol, 6.11% yield) as yellow solid. [M+H]⁺=375.1. Retention time=0.902 min.

Step 3:To a solution of 2-chloro-4-[2,5-difluoro-4-(trifluoromethyl)phenyl]-6,7-dimethyl-pteridine (1.00 eq, 10 mg, 0.0267 mmol) and (2S,6R)-2-(1-cyclopropylpyrazol-4-yl)-6-methyl-morpholine (1.00 eq, 5.5 mg, 0.0267 mmol) in DMSO (1 mL) was added DIEA (5.00 eq, 17 mg, 0.133 mmol), then the mixture was stirred at 100° C. for 1 h. LCMS showed reactant was consumed and 86% of desired mass was detected. the reaction solution was purified with Prep-HPLC(FA) and lyophilized to give (2S,6R)-2-(1-cyclopropylpyrazol-4-yl)-4-[4-[2,5-difluoro-4-(trifluoromethyl)phenyl]-6,7-dimethyl-pteridin-2-yl]-6-methyl-morpholine (7.4 mg, 0.0135 mmol, 50.62% yield) as yellow solid. [M+H]⁺=546.2. Retention time=1.037 min. 1H NMR (400 MHz, CDCl₃) δ=7.58-7.52 (m, 3H), 7.48 (dd, J=5.5, 8.6 Hz, 1H), 5.16-4.79 (m, 2H), 4.61 (br d, J=10.8 Hz, 1H), 3.90-3.76 (m, 1H), 3.58 (td, J=3.6, 7.2 Hz, 1H), 3.09 (dd, J=11.1, 13.4 Hz, 1H), 2.86 (dd, J=10.8, 13.1 Hz, 1H), 2.73 (s, 3H), 2.60 (s, 3H), 1.34 (d, J=6.2 Hz, 3H), 1.15-1.08 (m, 2H), 1.01 (s, 2H).

### Synthesis of I-1552 (Same General Method for I-1562)

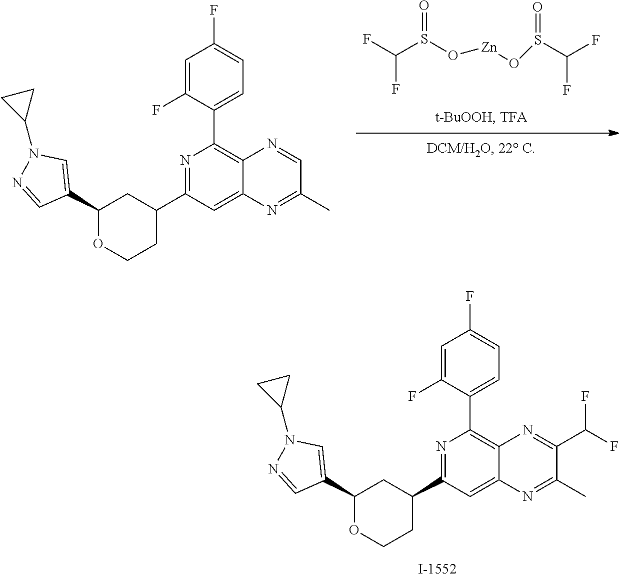

A glass vial equipped with a Teflon-coated magnetic stirring bar was charged with 7-((2R)-2-(1-cyclopropyl-1H-pyrazol-4-yl)tetrahydro-2H-pyran-4-yl)-5-(2,4-difluorophenyl)-2-methylpyrido[3,4-b]pyrazine (prepared via Method 37)(1.0 equiv, 90 mg, 0.20 mmol), bis(difluoromethylsulfinyloxy)zinc (3.0 equiv, 178 mg, 0.60 mmol), DCM (1.0 mL) and water (0.2 mL). The vial was cooled in an ice bath at 0° C. with stirring. Trifluoroacetic acid (1.0 equiv, 15.5 μL, 0.20 mmol) was added followed by tert-butyl hydroperoxide (70% solution in H₂O) (5.0 equiv, 0.14 mL, 0.50 mmol) dropwise. After 5 min, the ice bath was removed, and the reaction mixture was stirred at 22° C. for 36 h. The reaction mixture was diluted with DCM and washed with sat. NaHCO₃ (aq). The organic layer was dried over Na₂SO₄, filtered and evaporated to dryness under reduced pressure. The crude residual material was purified silica gel flash chromatography (Teledyne RediSep® GOLD column, 24 g SiO₂) using an elution gradient of 0% to 10% MeOH in DCM to yield 44 mg of crude product. The product was further purified by preparative HPLC (Gemini® 5 um NX-C18 110 Å, 100×30 mm column) using an elution gradient of MeCN in 10 mM aqueous ammonium formate pH 3.8 (40-60%) to afford 7-((2R,4S)-2-(1-cyclopropyl-1H-pyrazol-4-yl)tetrahydro-2H-pyran-4-yl)-3-(difluoromethyl)-5-(2,4-difluorophenyl)-2-methylpyrido[3,4-b]pyrazine (14 mg, 0.028 mmol, 14% yield) as an off-white solid. LC-MS(ESI⁺): Tr=1.72 min; [M+H]⁺ 498.1 (obs). ¹H NMR (DMSO-d₆, 400 MHz): δ_{H} 7.96 (1H, s), 7.73-7.78 (2H, m), 7.40-7.46 (2H, m), 7.11-7.38 (2H, m), 4.50 (1H, d, J=11.1 Hz), 4.10 (1H, d, J=11.3 Hz), 3.61-3.72 (2H, m), 3.37-3.40 (1H, m), 2.86 (3H, s), 2.23 (1H, d, J=13.0 Hz), 1.88-1.99 (3H, m), 0.90-0.99 (4H, m).

### Synthesis of 1-1557

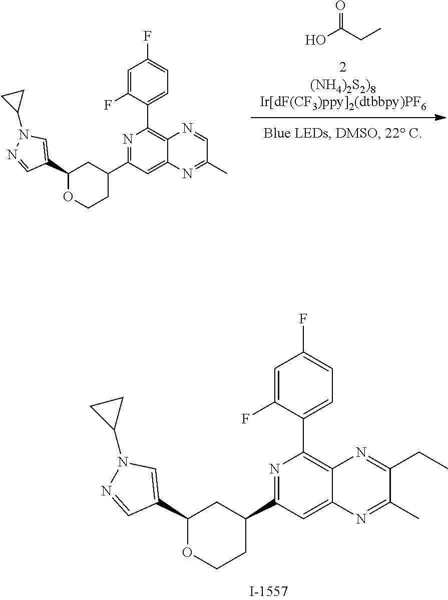

A flame-dried glass vial equipped with a Teflon-coated magnetic stirring bar was charged with 7-((2R)-2-(1-cyclopropyl-1H-pyrazol-4-yl)tetrahydro-2H-pyran-4-yl)-5-(2,4-difluorophenyl)-2-methylpyrido[3,4-b]pyrazine (prepared via Method 37)(1.0 equiv, 118 mg, 0.26 mmol), propionic acid 2 (10 equiv, 0.20 mL, 2.64 mmol), ammonium persulfate (2.0 equiv, 120 mg, 0.53 mmol), Ir[dF(CF₃)ppy]₂(dtbbpy)PF₆ (0.02 equiv, 5.9 mg, 0.005 mmol) and anhydrous DMSO (1.5 mL). The vial was sealed and degassed with Ar for 5 min. The reaction mixture was irradiated with Blue LEDs (450 nm) in a Penn PhD Photoreactor (25% lamp intensity) for 16 h at 22° C. with stirring. The crude reaction mixture was directly purified by reverse phase flash chromatography (Biotage® C18 duo column, 30 g) using an elution gradient of 10% to 100% MeCN in water with 0.1% formic acid to yield 66 mg of crude product as a brown solid. The product was further purified by preparative HPLC (Gemini® 5 um NX-C18 110 Å, 100×30 mm column) using an elution gradient of MeCN in 10 mM aqueous ammonium formate pH 3.8 (45-65%) to afford 7-((2R,4S)-2-(1-cyclopropyl-1H-pyrazol-4-yl)tetrahydro-2H-pyran-4-yl)-5-(2,4-difluorophenyl)-3-ethyl-2-methylpyrido[3,4-b]pyrazine (5.7 mg, 0.012 mmol, 4.5% yield) as an off-white solid. LC-MS(ESI⁺): Tr=1.74 min; [M+H]⁺ 476.2 (obs). ¹H NMR (DMSO-d6, 400 MHz): ^{6H} 7.83 (1H, s), 7.70-7.76 (2H, m), 7.36-7.42 (2H, m), 7.24-7.28 (1H, m), 4.49 (1H, dd, J=11.2, 2.0 Hz), 4.09 (1H, dd, J=11.2, 3.9 Hz), 3.62-3.72 (2H, m), 3.32 (2H, dd, J=11.2, 11.2 Hz), 2.98 (2H, q, J=7.3 Hz), 2.74 (3H, s), 2.22 (1H, d, J=13.0 Hz), 1.86-1.98 (3H, m), 1.19 (3H, t, J=7.3 Hz), 0.90-1.02 (4H, m).

### Synthesis of I-1567

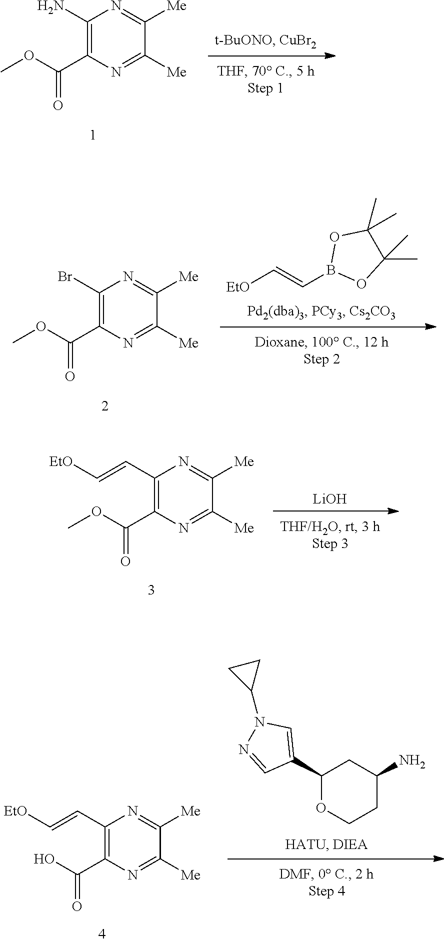

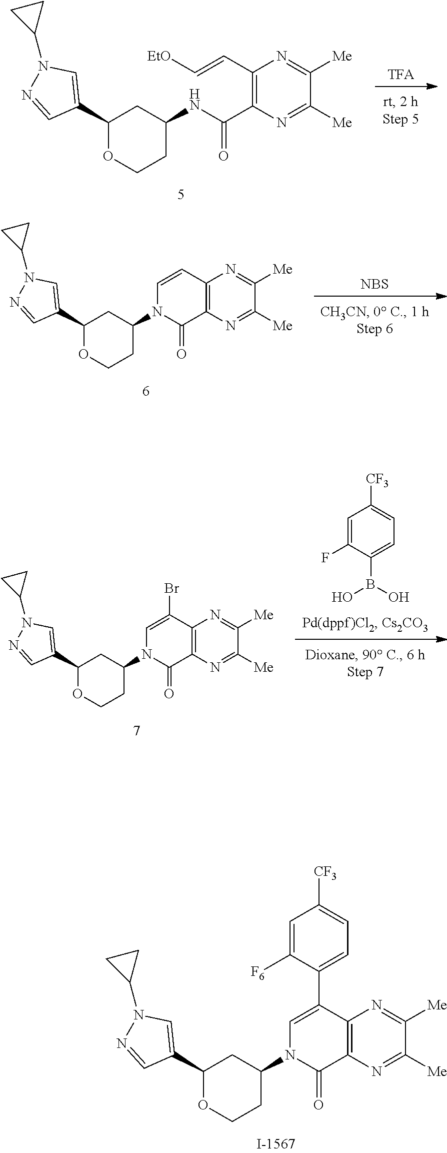

Step 1: To a solution of CuBr₂ (2.95 g, 13.25 mmol, 1.5 equiv) in THF (30 mL) was added t-BuONO (1.37 g, 13.25 mmol, 1.5 equiv) at room temperature under nitrogen. After stirring at 70° C. for 10 minutes, the solution was cooled to room temperature and a solution of methyl 3-amino-5,6-dimethylpyrazine-2-carboxylate (1.60 g, 8.83 mmol, 1.0 equiv) in THF (10 mL) was added dropwise. The mixture was then stirred at 70° C. for 2 hours. LCMS indicated the starting material was consumed completely and 60% desired compound was detected. The reaction mixture was cooled to room temperature, quenched with water, and extracted with ethyl acetate (30 mL×3). The organic phase was dried over Na₂SO₄ and concentrated under reduced pressure. The crude product was purified by column chromatography (SiO₂, Petroleum ether: ethyl acetate=0% to 20%) to give the product methyl 3-bromo-5,6-dimethylpyrazine-2-carboxylate (879 mg, 3.59 mmol, 40% yield) as a yellow solid. ¹H NMR (400 MHz, DMSO) δ 3.91 (s, 3H), 2.54 (s, 3H), 2.49 (s, 3H).

Step 2: A mixture of Pd₂(dba)₃ (176 mg, 0.3 mmol, 0.1 equiv), PCy₃ (172 mg, 0.62 mmol, 0.2 equiv), Cs₂CO₃ (3.98 g, 12.2 mmol, 3.0 equiv) and methyl 3-bromo-5,6-dimethyl-pyrazine-2-carboxylate (750 mg, 3.06 mmol, 1.0 equiv) was prepared in a flask under nitrogen. Then 1,4-dioxane (20 mL) and 2-[(E)-2 ethoxyvinyl]-4,4,5,5-tetramethyl-1,3,2-dioxaborolane (909 mg, 4.59 mmol, 1.5 equiv.) were added and the mixture was stirred at 100° C. for 5 h. LCMS indicated the starting material was consumed completely and 80% desired compound was detected. Then the suspension was cooled to room temperature, filtered through a plug of celite, washed with water and extracted with EtOAc. The organic phase was dried over Na₂SO₄ and concentrated under reduced pressure. The crude product was purified by column chromatography (silica gel, EtOAc/PE=1:1) to give the product methyl (E)-3-(2-ethoxyvinyl)-5,6-dimethylpyrazine-2-carboxylate (520 mg, 2.20 mmol, 72% yield) as a yellow solid. ¹H NMR (400 MHz, DMSO) δ 7.74 (d, J=6.4 Hz, 1H), 6.52 (d, J=8.0 Hz, 1H), 4.00 (q, J=8.13 Hz, 2H), 3.85 (s, 3H), 2.49 (s, 3H), 2.44 (s, 3H), 1.28 (t, J=7 Hz, 3H).

Step 3: To a solution of methyl (E)-3-(2-ethoxyvinyl)-5,6-dimethylpyrazine-2-carboxylate (520 mg, 2.20 mmol, 1.0 equiv) in THF (15 mL) and water (5 mL) was added LiOH (102 mg, 4.40 mmol, 2.0 equiv). The mixture was stirred at room temperature for 3 hours. LCMS indicated that the starting material was consumed completely, and 80% desired compound was detected. The resulting solution was treated with HCl to pH 5 and dried by a freeze dryer to give the product (E)-3-(2-ethoxyvinyl)-5,6-dimethylpyrazine-2-carboxylic acid. The crude product was used for the next step without further purification. LCMS: Rt: 1.335 min, m/z: [M+H]⁺=223.1. 80% purity at 254 nm.

Step 4: A solution of 3-[(E)-2-ethoxyvinyl]-5,6-dimethyl-pyrazine-2-carboxylic acid (333 mg, 1.5 mmol, 1.0 equiv), 2-(1-cyclopropylpyrazol-4-yl)tetrahydropyran-4-amine (310 mg, 1.5 mmol, 1.0 equiv) and HATU (856 mg, 2.25 mmol, 1.5 equiv) in DMF (20 mL) was prepared in a flask under nitrogen. Then DIEA (582 mg, 4.5 mmol, 3.0 equiv) was added and the solution was stirred at 0° C. for 1 h. LCMS indicated that the starting material was consumed, and the desired compound was detected. The reaction was quenched with water and extracted with EtOAc. The organic phase was washed with brine, dried over Na₂SO₄ and concentrated under reduced pressure. The crude product was purified by column chromatography (silica, Petroleum ether/Ethyl acetate=1:2) to give the desired product N-[(2R,4S)-2-(1-cyclopropyl-1H-pyrazol-4-yl)tetrahydro-2H-pyran-4-yl]-3-[(E)-2-ethoxyvinyl]-5,6-dimethylpyrazine-2-carboxamide (480 mg, 1.17 mmol, 78% yield) as a yellow oil. ¹H NMR (400 MHz, DMSO) δ 8.50 (d, J=8.0 Hz, 1H), 7.72-7.66 (m, 2H), 7.34 (s, 1H), 6.88 (d, J=12.4 Hz, 1H), 4.38 (d, J=10.4 Hz, 1H), 4.15-4.04 (m, 1H), 3.99-3.93 (m, 2H), 3.68-3.61 (m, 1H), 3.56 (t, J=11.4 Hz, 1H), 2.56 (d, J=12.4 Hz, 1H), 2.47 (s, 3H), 2.47 (s, 3H), 2.05 (d, J=15.2 Hz, 1H), 1.78 (d, J=11.6 Hz, 1H), 1.67-1.58 (m, 2H), 1.29-1.24 (m, 3H), 0.99-0.88 (m, 4H).

Step 5: A solution of N-[(2R,4S)-2-(1-cyclopropylpyrazol-4-yl)tetrahydropyran-4-yl]-3-[(E)-2-ethoxyvinyl]-5,6-dimethyl-pyrazine-2-carboxamide (480 mg, 1.17 mmol, 1.0 equiv) in TFA (2.0 mL) was prepared under nitrogen. Then the reaction was stirred at room temperature for 3 hours. LCMS indicated that the starting material was consumed completely, and desired compound was detected. The reaction was quenched with water and extracted with EtOAc. The organic phase was dried over Na₂SO₄ and concentrated under reduced pressure. The crude product was purified by column chromatography (silica gel, DCM/MeOH=10:1) to give 6-[(2R,4S)-2-(1-cyclopropyl-1H-pyrazol-4-yl)tetrahydro-2H-pyran-4-yl]-2,3-dimethylpyrido[3,4-b]pyrazin-5(6H)-one as a brown solid (262 mg, 0.72 mmol, 61% yield). ¹H NMR (400 MHz, CDCl₃) δ 7.86 (d, J=7.6 Hz, 1H), 7.74 (s, 1H), 7.40 (s, 1H), 6.66 (d, J=8.0 Hz, 1H), 5.22-5.12 (m, 1H), 4.54-4.51 (m, 1H), 4.09-4.06 (m, 1H), 3.74-3.65 (m, 2H), 2.63 (s, 6H), 2.06-2.01 (m, 3H), 1.77 (d, J=9.6 Hz, 1H), 0.98-0.90 (m, 4H).

Step 6: To a solution of 6-[(2R,4S)-2-(1-cyclopropyl-1H-pyrazol-4-yl)tetrahydro-2H-pyran-4-yl]-2,3-dimethylpyrido[3,4-b]pyrazin-5(6H)-one (240 mg, 0.66 mmol, 1.0 equiv) in MeCN (10 mL) was added NBS (129 mg, 0.72 mmol, 1.1 equiv) under nitrogen. The solution was stirred at 0° C. for 1 h. LCMS indicated that the starting material was consumed and 50% of the desired product was detected. The reaction was quenched with water and extracted with EtOAc. The organic phase was dried over Na₂SO₄ and concentrated under reduced pressure. The crude product was purified by column chromatography (silica gel, EtOAc/Et₃N=20: 1) to give 8-bromo-6-[(2R,4S)-2-(1-cyclopropyl-1H-pyrazol-4-yl)tetrahydro-2H-pyran-4-yl]-2,3-dimethylpyrido[3,4-b]pyrazin-5(6H)-one (57 mg, 0.13 mmol, 20% yield) as a white solid. ¹H NMR (400 MHz, DMSO) δ 8.22 (s, 1H), 7.75 (s, 1H), 7.41 (s, 1H), 5.20-5.12 (m, 1H), 4.52 (dd, J=1.6, 10.8 Hz, 1H), 4.08-4.05 (m, 1H), 3.71-3.63 (m, 2H), 2.69 (s, 3H), 2.67 (s, 3H), 2.17 (q, J=11.9 Hz, 1H), 2.03-2.01 (m, 1H), 1.78 (d, J=12.4 Hz, 1H), 1.18 (t, J=7.0 Hz, 1H), 1.01-0.91 (m, 4H).

Step 7: A mixture of 8-bromo-6-[(2R,4S)-2-(1-cyclopropyl-1H-pyrazol-4-yl)tetrahydro-2H-pyran-4-yl]-2,3-dimethylpyrido[3,4-b]pyrazin-5(6H)-one (50 mg, 0.11 mmol, 1.0 equiv), [2-fluoro-4-(trifluoromethyl)phenyl]boronic acid (47 mg, 0.23 mmol, 2.0 equiv), Pd(dppf)Cl₂ (8 mg, 0.01 mmol, 0.1 equiv) and Cs₂CO₃ (110 mg, 0.34 mmol, 3.0 equiv) was prepared in a flask under nitrogen, then 1,4-dioxane was added. The solution was stirred at 90° C. for 3 hours. LCMS indicated that the starting material was consumed, and desired product was detected. The suspension was filtered through a plug of silica gel and concentrated under reduced pressure. The crude product was purified by column chromatography (silica gel, EtOAc/Et₃N=20:1) to give the product 6-[(2R,4S)-2-(1-cyclopropyl-1H-pyrazol-4-yl)tetrahydro-2H-pyran-4-yl]-8-[2-fluoro-4-(trifluoromethyl)phenyl]-2,3-dimethylpyrido[3,4-b]pyrazin-5(6H)-one (22 mg, 0.04 mmol, 37% yield) was brown solid. LC-MS: Rt: 1.37 min, m/z: 528.2 [M+H]⁺. 99% purity at 254 nm. ¹H NMR (400 MHz, DMSO) δ 8.03 (s, 1H), 7.79-7.75 (m, 2H), 7.73 (s, 1H), 7.69 (d, J=8.0 Hz, 1H), 7.40 (s, 1H), 5.29-5.22 (m, 1H), 4.55 (dd, J=1.2, 10.8 Hz, 1H), 3.08 (dd, J=3.2, 11.2 Hz, 1H), 3.72 (t, J=11.0 Hz, 1H), 3.67-3.61 (m, 1H), 2.65 (s, 3H), 2.55 (s, 3H), 2.18 (q, 11.7 Hz, 1H), 2.07-2.01 (m, 2H), 1.83 (d, J=12.0 Hz, 1H), 0.98-0.86 (m, 4H). HPLC: Rt: 3.85 min, 98% purity at 214 nm.

### Synthesis of Compound I-1572

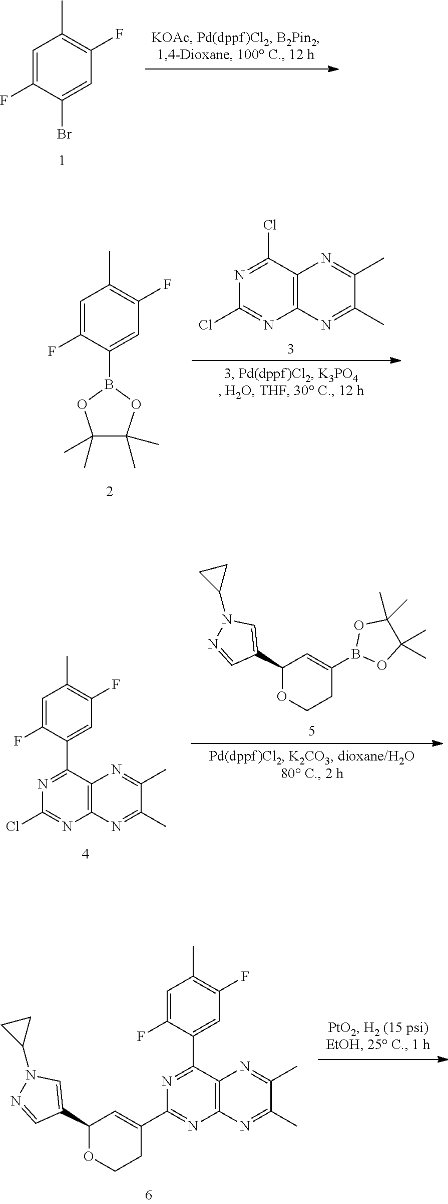

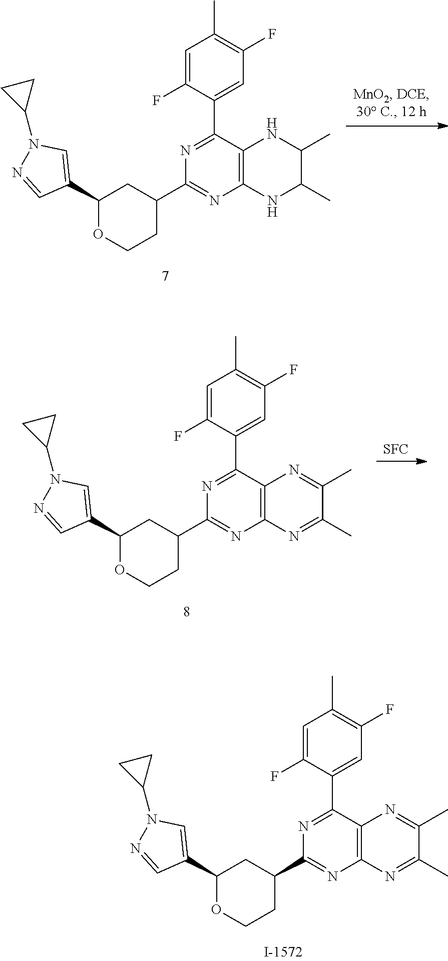

Step 1: To a solution of 1-bromo-2,5-difluoro-4-methyl-benzene (1.00 eq, 160 mg, 0.773 mmol) in 1,4-Dioxane (5 mL) were added 4,4,5,5-tetramethyl-2-(4,4,5,5-tetramethyl-1,3,2-dioxaborolan-2-yl)-1,3,2-dioxaborolane (1.50 eq, 0.29 g, 1.16 mmol) and KOAc (2.50 eq, 190 mg, 1.93 mmol), then Pd(dppf)Cl₂·CH₂Cl₂ (0.100 eq, 63 mg, 0.0773 mmol) was added to the mixture under N₂. The reaction was stirred at 100° C. for 10 h. TLC (PE/EA=10/1, starting material Rf=0.4, new spot Rf=0.6) indicated Reactant was consumed completely and one new spot formed. The reaction mixture was filtered and the filter cake was washed with DCM (5 mL), combined the solvent and evaporate to give the residue. The residue was purified by column chromatography (PE/ethyl acetate=0 to 10%, PE/EtOAc=10/1, the desired product Rf=0.6) to obtain 2-(2,5-difluoro-4-methyl-phenyl)-4,4,5,5-tetramethyl-1,3,2-dioxaborolane (50 mg, 0.197 mmol, 25.46% yield) as white solid. ¹H NMR (400 MHz, CDCl₃, 298 K) Shift (ppm)=7.32 (dd, J=4.7, 9.3 Hz, 1H), 6.86 (dd, J=5.8, 8.9 Hz, 1H), 2.33-2.24 (m, 3H), 1.36 (s, 12H).

Step 2: A sealed bottle under N₂ atmosphere was charged with 2,4-dichloro-6,7-dimethyl-pteridine (1.00 eq, 325 mg, 1.42 mmol), 2-(2,5-difluoro-4-methyl-phenyl)-4,4,5,5-tetramethyl-1,3,2-dioxaborolane (1.00 eq, 360 mg, 1.42 mmol), Pd(dppf)Cl₂ (0.1000 eq, 104 mg, 0.142 mmol) and K₃PO₄ (3.00 eq, 902 mg, 4.25 mmol) and purged with N₂ three times, then THF (10 mL) and H₂O (5.00 eq, 128 mg, 7.08 mmol) was added in one portion at 15° C., then the mixture was stirred at 15° C. for 1 h and 30° C. for 10 h. The reaction solution was changed from orange to dark purple, LCMS showed raw material was consumed completely and the major peak showed MS (32%, Rt: 0.918 min; [M+H]⁺=321.0 at 220 nm). The reaction mixture was evaporated to give the residue. The residue was purified by column chromatography (PE/ethyl acetate=0 to 20%, PE/EtOAc=10/1, the desired product Rf=0.3) to obtain 2-chloro-4-(2,5-difluoro-4-methyl-phenyl)-6,7-dimethyl-pteridine (120 mg, 0.374 mmol, 26.41% yield) as white solid and 150 mg crude product (40% purity) as white solid. [M+H]⁺=321.0; purity=75% (220 nm). Retention time=0.941 min.

Step 3: To a solution of 2-chloro-4-(2,4-difluoro-5-methyl-phenyl)-6,7-dimethyl-pteridine (1.00 eq, 120 mg, 0.374 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.00 eq, 118 mg, 0.374 mmol) and K₂CO₃ (3.00 eq, 94 mg, 1.12 mmol) in 1,4-Dioxane (10 mL) and Water (1 mL) was added Pd(dppf)Cl₂ DCM (0.120 eq, 33 mg, 0.0449 mmol) in one portion. Then the reaction mixture was stirred at 80° C. for 12 hours under N2 atmosphere. LCMS showed the starting material was consumed completely and desired product was detected (70%, Rt: 0.987 min; [M+H]⁺=475.1 at 220 nm). The mixture was poured into 30 mL H₂O, extracted with EA (50 mL*2), the combine organic layers was dried over anhydrous Na₂SO₄, filtered and concentrated under reduced pressure to give the residue. The residue was purified by Prep-TLC (PE:EA=1:2, Rf=0.6) to give the 2-[(6R)-6-(1-cyclopropylpyrazol-4-yl)-3,6-dihydro-2H-pyran-4-yl]-4-(2,4-difluoro-5-methyl-phenyl)-6,7-dimethyl-pteridine (100 mg, 0.211 mmol, 56% yield) as yellow solid. [M+H]⁺=475.2; purity=95% (220 nm). Retention time=0.893 min. 1H NMR (400 MHz, CDCl3, 298 K) Shift (ppm)=7.67 (d, J=1.7 Hz, 1H), 7.54 (s, 1H), 7.51 (s, 1H), 7.44 (dd, J=5.6, 9.2 Hz, 1H), 7.08 (dd, J=6.1, 9.7 Hz, 1H), 5.47 (br d, J=2.6 Hz, 1H), 4.18-4.09 (m, 1H), 3.99-3.90 (m, 1H), 3.57 (tt, J=3.6, 7.3 Hz, 1H), 3.05-2.91 (m, 2H), 2.85 (s, 3H), 2.74 (s, 3H), 2.39 (d, J=1.1 Hz, 3H), 1.15-1.09 (m, 2H), 1.04-0.96 (m, 2H).

Step 4: To a solution of 2-[(6R)-6-(1-cyclopropylpyrazol-4-yl)-3, 6-dihydro-2H-pyran-4-yl]-4-(2,5-difluoro-4-methyl-phenyl)-6,7-dimethyl-pteridine (1.00 eq, 120 mg, 0.253 mmol) in Ethanol (1 mL) was added PtO₂ (1.00 eq, 57 mg, 0.253 mmol) under N₂ atmosphere. The mixture was purged with H₂ (15 psi) 3 times, then the mixture was stirred at 30° C. for 16 hour under H₂ (15 psi) atmosphere. LCMS showed the starting material was consumed completely and desired product was detected (97%, Rt: 0.798 min; [M+H]⁺=481.3 at 220 nm). The suspension was filtered through a pad of celite or silica gel and the filter cake was washed with EtOH (5 mL×3). The combined filtrates were concentrated to dryness to give 2-[(2R)-2-(1-cyclopropylpyrazol-4-yl)tetrahydropyran-4-yl]-4-(2,5-difluoro-4-methyl-phenyl)-6,7-dimethyl-5,6,7,8-tetrahydropteridine (120 mg, 0.230 mmol, 90.84% yield) as yellow gum (120 mg), checked by LCMS [M+H]⁺=481.3; purity=92% (220 nm). Retention time=0.713 min. 1H NMR (400 MHz, CDCl3) δ=7.49-7.43 (m, 2H), 7.21 (dd, J=5.9, 9.4 Hz, 1H), 6.99 (dd, J=6.0, 9.8 Hz, 1H), 4.44 (dd, J=1.8, 11.3 Hz, 1H), 3.54 (tt, J=3.8, 7.3 Hz, 2H), 2.97 (tt, J=3.7, 11.9 Hz, 1H), 2.32 (d, J=1.6 Hz, 3H), 2.28-2.19 (m, 1H), 2.20-2.08 (m, 2H), 2.03-1.84 (m, 4H), 1.21 (d, J=6.5 Hz, 3H), 1.13 (d, J=6.4 Hz, 3H), 1.10-1.07 (m, 2H), 0.99-0.95 (m, 2H).

Step 5: To a solution of 2-[(2R)-2-(1-cyclopropylpyrazol-4-yl)tetrahydropyran-4-yl]-4-(2,5-difluoro-4-methyl-phenyl)-6,7-dimethyl-pteridine (1.00 eq, 99 mg, 0.208 mmol) in DCE (1 mL) was added MnO₂ (20.0 eq, 362 mg, 4.16 mmol). The mixture was stirred at 30° C. for 48 h. LCMS showed the starting material was consumed completely and desired product was detected (96%, Rt: 0.936 min; [M+H]⁺=477.2 at 220 nm). The reaction was filtered and the filtrate was concentrated in vacuum. The crude product was purified by SFC (0.10% NH₃H₂O IPA B: 40%-40%; Detector, UV 254 nm. RT: 5.4 min)). After SFC separation, the eluent was concentrated to remove organic solvents. The residual aqueous solution was lyophilized to give 2-[(2R,4S)-2-(1-cyclopropylpyrazol-4-yl)tetrahydropyran-4-yl]-4-(2,5-difluoro-4-methyl-phenyl)-6,7-dimethyl-pteridine (42 mg, 0.0608 mmol, 29.23% yield) as white solid. The purity was 81% under 254 nm and 69% under 215 nm in HPLC. The product was purified by prep-TLC (PE/EA=2/1, rf=0.5) to give 2-[(2R,4S)-2-(1-cyclopropylpyrazol-4-yl)tetrahydropyran-4-yl]-4-(2,5-difluoro-4-methyl-phenyl)-6,7-dimethyl-pteridine (29 mg, 0.0609 mmol, 29.25% yield) as white solid. 100%, Rt: 0.956 min; [M+H]⁺=477.2 at 220 nm) purity, 90% under 215 nm and 94% under 254 nm in HPLC; 1H NMR (400 MHz, CDCl3) δ=7.51 (s, 2H), 7.42 (dd, J=5.6, 9.1 Hz, 1H), 7.08 (dd, J=6.1, 9.4 Hz, 1H), 4.56 (dd, J=1.9, 11.4 Hz, 1H), 4.27 (td, J=3.1, 11.1 Hz, 1H), 3.89-3.77 (m, 1H), 3.62-3.48 (m, 2H), 2.85 (s, 3H), 2.74 (s, 3H), 2.45 (br d, J=13.4 Hz, 1H), 2.40 (d, J=1.6 Hz, 3H), 2.24-2.16 (m, 3H), 1.15-1.07 (m, 2H), 1.03-0.94 (m, 2H).

### Synthesis of Compounds I-1577 and I-1578

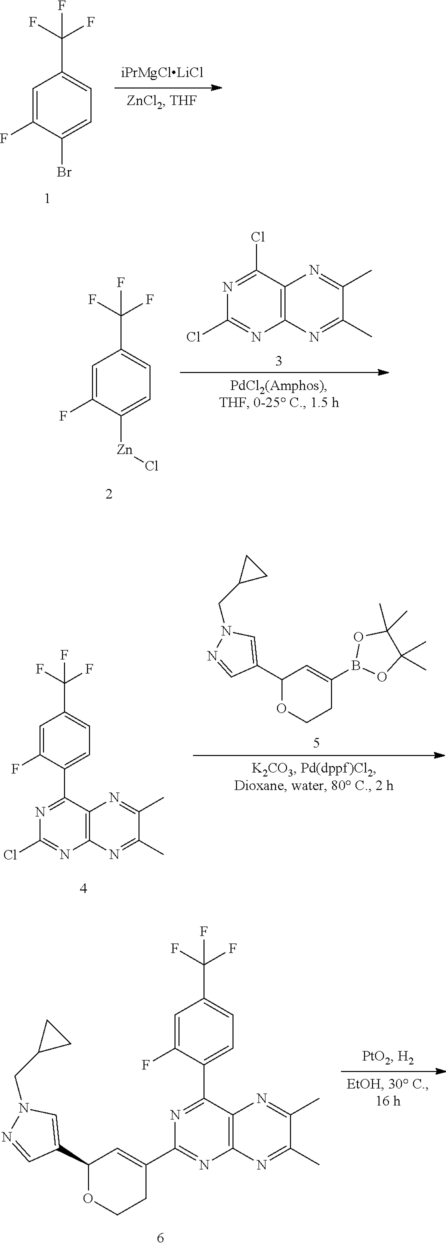

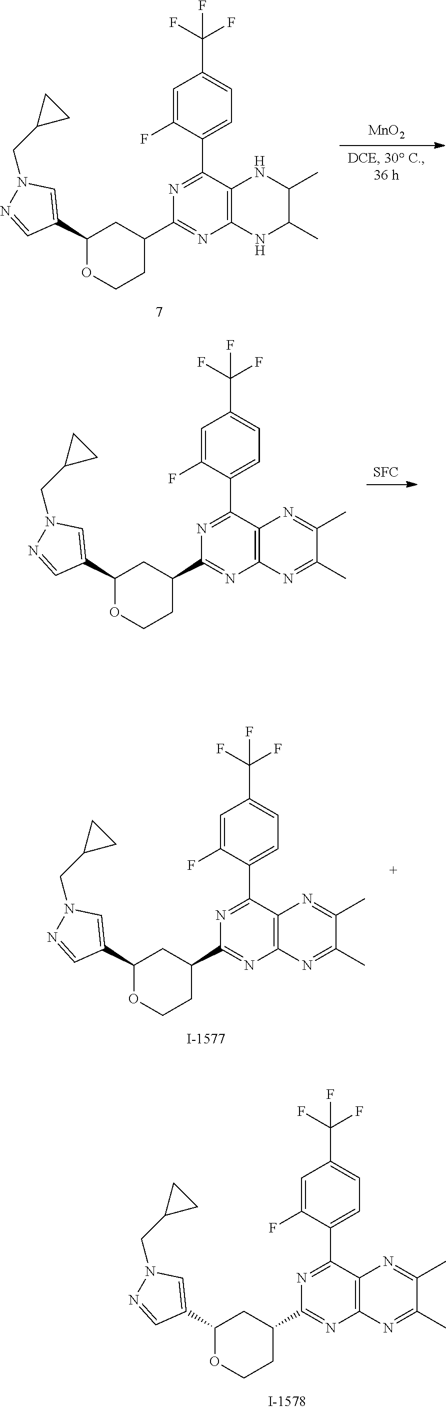

Step 1: To a solution of 1-bromo-2-fluoro-4-(trifluoromethyl)benzene (1.00 eq, 3000 mg, 12.3 mmol) in THF (30 mL) was added iPrMgCl LiCl (1.11 eq, 11 mL, 13.7 mmol) at 0° C. under N₂. The mixture was stirred at 15° C. for 1.5 hours. ZnCl₂ (1.21 eq, 30 mL, 15.0 mmol) was added at −78° C. under N₂ and the mixture was stirred at 15° C. for 1 hour. The reaction mixture was used directly for the next step.

Step 2: A sealed bottle under N₂ atmosphere was charged with 2,4-dichloro-6,7-dimethyl-pteridine (1.00 eq, 300 mg, 1.31 mmol) and PdCl₂(Amphos) (0.0500 eq, 46 mg, 0.0655 mmol) and THF (3 mL) and purged with N₂ three times, then cooled to 0° C., chloro-[2-fluoro-4-(trifluoromethyl)phenyl]zinc (1.20 eq, 415 mg, 1.57 mmol) was added dropwise to the reaction solution at 0° C., then warmed to 25° C. and stirred for 1 hour. The reaction solution was changed from orange to dark purple, LCMS showed 60% of the desired product was detected (MS: 357.1 [M+H]⁺,ESI pos, RT=0.948 min). The reaction solution was quenched with saturated NH₄Cl solution (100 mL), then extracted with EtOAc (500 mL) and evaporated under reduced pressure to give the residue, which was then purified with Flash column (PE:EA=0 to 40%, PE:EA=3:1, Rf=0.5) and dried in vacuo to give 2-chloro-4-[2-fluoro-4-(trifluoromethyl)phenyl]-6,7-dimethyl-pteridine (330 mg, 0.712 mmol, 54.39% yield) as red solid. (M+H)⁺=357.2; purity=77% (220 nm). Retention time=0.958 min. ¹H NMR (400 MHz, CDCl3) δ=7.89 (t, J=7.3 Hz, 1H), 7.64 (d, J=8.1 Hz, 1H), 7.53 (d, J=9.8 Hz, 1H), 2.87 (s, 4H), 2.75 (s, 3H).

Step 3: To a solution of 2-chloro-4-[2-fluoro-4-(trifluoromethyl)phenyl]-6,7-dimethyl-pteridine (1.00 eq, 280 mg, 0.785 mmol) K₂CO₃ (3.00 eq, 325 mg, 2.35 mmol) and Pd(dppf)Cl₂·CH₂Cl₂ (0.1000 eq, 64 mg, 0.0785 mmol) in 1,4-Dioxane (5 mL) and Water (0.5000 mL) was added 1-(cyclopropylmethyl)-4-[4-(4,4,5,5-tetramethyl-1,3,2-dioxaborolan-2-yl)-3,6-dihydro-2H-pyran-6-yl]pyrazole (1.50 eq, 389 mg, 1.18 mmol). The mixture was stirred at 80° C. for 2 hours. LCMS showed the starting material was consumed completely and a major peak with desired product mass (37%, MS: 525.0 [M+H]⁺, ESI pos). The mixture was extracted with EA (100 mL). The combined organic layer concentrated under reduced pressure to get the crude residue. The residue was filtered and the filtrate was purified by prep-HPLC (flow: 25 mL/min; gradient: from 38-68% water (0.1% FA)-ACN over 7 min; column: Unisil 3-100 C18 Ultra 150*25 mm*10 um) and lyophilized to afford 4-[2-fluoro-4-(trifluoromethyl)phenyl]-6,7-dimethyl-2-[rac-(6R)-6-[1-(cyclopropylmethyl)pyrazol-4-yl]-3,6-dihydro-2H-pyran-4-yl]pteridine (120 mg, 0.229 mmol, 29.15% yield) as gray solid. (M+H)⁺=525.0; purity=94% (220 nm). Retention time=1.009 min. ¹H NMR (400 MHz, CDCl3) δ ppm 0.37 (q, J=5.09 Hz, 2H) 0.60-0.69 (m, 2H) 1.22-1.36 (m, 1H) 2.74 (s, 3H) 2.86 (s, 3H) 3.97 (d, J=7.00 Hz, 2H) 4.13-4.21 (m, 1H) 5.51 (br d, J=2.50 Hz, 1H) 7.50-7.59 (m, 3H) 7.70 (d, J=1.88 Hz, 1H) 7.89 (t, J=7.25 Hz, 1H).

Step 4: To a solution of 2-[(6R)-6-[1-(cyclopropylmethyl) pyrazol-4-yl]-3, 6-dihydro-2H-pyran-4-yl]-4-[2-fluoro-4-(trifluoromethyl) phenyl]-6, 7-dimethyl-pteridine (1.00 eq, 110 mg, 0.210 mmol) in Ethanol (4 mL) was added PtO₂ (0.434 eq, 21 mg, 0.0910 mmol) under N₂. The suspension was degassed under vacuum and purged with H₂ five times. The mixture was stirred under H₂ (15 psi) at 30° C. for 16 hours. LCMS showed the starting material was consumed completely and a new peak with desired product mass (96%, MS: 531.2 [M+H]⁺, ESI pos). The mixture was filtered through a Celite pad, and the filtrate was concentrated to give 2-[(2R)-2-[1-(cyclopropylmethyl) pyrazol-4-yl] tetrahydropyran-4-yl]-4-[2-fluoro-4-(trifluoromethyl) phenyl]-6, 7-dimethyl-5, 6, 7, 8-tetrahydropteridine (120 mg, 0.208 mmol, 99.22% yield) as brown solid. [M+2+H]⁺, 531.2; purity=92% (220 nm). Retention time=0.505 min. ¹H NMR (400 MHz, CDCl3) δ ppm 0.36 (q, J=5.09 Hz, 2H) 0.60-0.66 (m, 2H) 0.82-0.93 (m, 1H) 1.14 (d, J=6.38 Hz, 3H) 1.22 (d, J=6.75 Hz, 3H) 1.86-2.04 (m, 4H) 2.10 (s, 1H) 2.15-2.23 (m, 1H) 2.99 (tt, J=11.93, 3.71 Hz, 1H) 3.47 (br s, 1H) 3.73 (br d, J=7.00 Hz, 2H) 3.94 (d, J=7.00 Hz, 2H) 4.19 (br dd, J=11.26, 3.63 Hz, 1H) 4.48 (dd, J=11.38, 1.75 Hz, 1H) 5.37 (br s, 1H) 7.42-7.58 (m, 4H) 7.73 (t, J=7.38 Hz, 1H).

Step 5: To a solution of 2-[(2R)-2-[1-(cyclopropylmethyl) pyrazol-4-yl] tetrahydropyran-4-yl]-4-[2-fluoro-4-(trifluoromethyl) phenyl]-6, 7-dimethyl-5, 6, 7, 8-tetrahydropteridine (1.00 eq, 120 mg, 0.226 mmol) in DCE (5 mL) was added MnO₂ (20.0 eq, 393 mg, 4.52 mmol). The mixture was stirred at 30° C. for 12 hours. LCMS showed a new peak with desired mass, however, most of the starting material still remained unchanged. (38%, MS: 527.2 [M+H]⁺, ESI pos). The reaction was filtered and the MnO₂ (20.0 eq, 393 mg, 4.52 mmol) was added to the filtrate. The mixture was stirred at 30° C. for 24 hours. LCMS showed that no starting material remained and a major peak with desired product mass was present (84%, MS: 527.2 [M+H]⁺, ESI pos). The reaction was filtered and the filtrate was purified by prep-HPLC (flow: 25 mL/min; gradient: from 60-90% water (0.1% FA)-ACN over 10 min; column: YMC Triart C18 150*25 mm*5 um) and lyophilized to afford 2-[(2R)-2-[1-(cyclopropylmethyl)pyrazol-4-yl]tetrahydropyran-4-yl]-4-[2-fluoro-4-(trifluoromethyl)phenyl]-6,7-dimethyl-pteridine (30 mg, 0.0570 mmol, 25.20% yield) as white solid. [M+H]⁺, 527.4; purity=99.66% (220 nm). Retention time=0.984 min. HPLC: Retention time=2.464 min, 99.02% purity at 220 nm. ¹H NMR (400 MHz, CDCl3) δ ppm 0.38 (q, J=4.96 Hz, 2H) 0.61-0.69 (m, 2H) 1.22-1.34 (m, 1H) 2.17-2.33 (m, 3H) 2.48 (br d, J=13.38 Hz, 1H) 2.74 (s, 3H) 2.87 (s, 3H) 3.51-3.64 (m, 1H) 3.80-3.88 (m, 1H) 3.96 (d, J=7.00 Hz, 2H) 4.26-4.33 (m, 1H) 4.61 (dd, J=11.32, 1.81 Hz, 1H) 7.53 (t, J=4.19 Hz, 2H) 7.58 (s, 1H) 7.64 (d, J=7.88 Hz, 1H) 7.87 (t, J=7.19 Hz, 1H).

Step 6: The product mixture from step 4 was purified by SFC (DAICEL CHIRALPAK AD (250 mm*30 mm, 10 um), Mobile phase: Phase A for CO₂, and Phase B for MeOH (0.05% DEA); Gradient elution: 40% MeOH (0.05% DEA) in CO₂, Flow rate: 3 mL/min; Detector, PDA, Column Temp: 35° C.; Back Pressure: 100 Bar) to give 2-[(2R,4S)-2-[1-(cyclopropylmethyl)pyrazol-4-yl]tetrahydropyran-4-yl]-4-[2-fluoro-4-(trifluoromethyl)phenyl]-6,7-dimethyl-pteridine (18 mg, 0.0328 mmol, 14.50% yield) as white solid and the 2-[(2S,4R)-2-[1-(cyclopropylmethyl)pyrazol-4-yl]tetrahydropyran-4-yl]-4-[2-fluoro-4-(trifluoromethyl)phenyl]-6,7-dimethyl-pteridine (12 mg, 0.022 mmol, 9.71% yield) as white solid. LC-MS: Rt: 0.984 min, m/z: 527.4 [M+H]⁺. 99.85% purity at 220 nm. HPLC: Retention time=2.461 min, 96.13% purity at 220 nm. ¹H NMR (400 MHz, CDCl3) δ ppm 0.33-0.42 (m, 2H) 0.61-0.70 (m, 2H) 1.25-1.30 (m, 1H) 2.19-2.32 (m, 3H) 2.43-2.53 (m, 1H) 2.74 (s, 3H) 2.87 (s, 3H) 3.57 (ddd, J=15.79, 11.91, 3.81 Hz, 1H) 3.78-3.88 (m, 1H) 3.96 (d, J=7.13 Hz, 2H) 4.24-4.33 (m, 1H) 4.57-4.64 (m, 1H) 7.53 (t, J=4.13 Hz, 2H) 7.58 (s, 1H) 7.63 (d, J=8.13 Hz, 1H) 7.87 (t, J=7.19 Hz, 1H). LC-MS: Rt: 0.984 min, m/z: 527.4 [M+H]⁺. 99.35% purity at 220 nm. HPLC: Retention time=2.467 min, 98.84% purity at 220 nm. ¹H NMR (400 MHz, CDCl3) δ ppm 0.37 (q, J=4.92 Hz, 2H) 0.61-0.69 (m, 2H) 1.24-1.30 (m, 1H) 2.16-2.33 (m, 3H) 2.44-2.52 (m, 1H) 2.74 (s, 3H) 2.87 (s, 3H) 3.51-3.63 (m, 1H) 3.79-3.88 (m, 1H) 3.96 (d, J=7.00 Hz, 2H) 4.25-4.33 (m, 1H) 4.60 (dd, J=11.38, 1.88 Hz, 1H) 7.53 (t, J=4.13 Hz, 2H) 7.58 (s, 1H) 7.63 (d, J=8.00 Hz, 1H) 7.87 (t, J=7.25 Hz, 1H).

### Synthesis of Compound I-1582

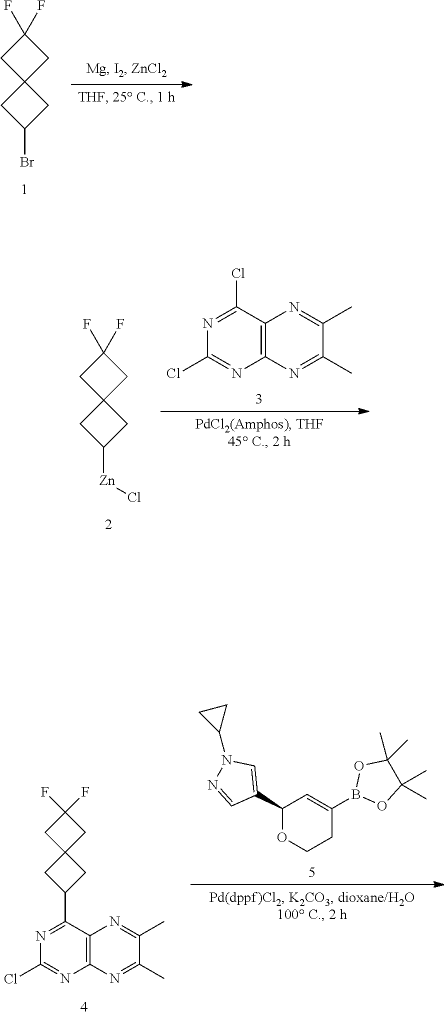

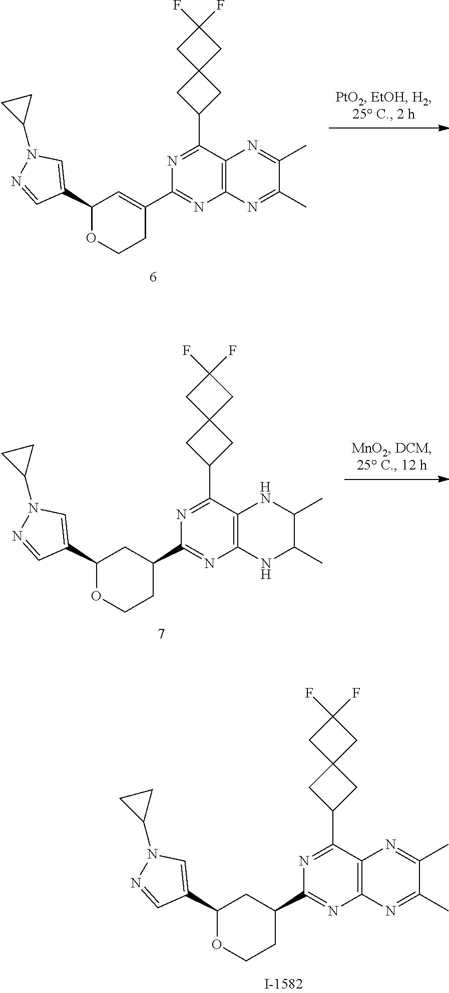 [SMILES: Cc1nc2nc(C3=C[C@H](c4cnn(C5CC5)c4)OCC3)nc(C3CC4(C3)CC(F)(F)C4)c2nc1C ; "PtO2, EtOH, H2,25° C., 2 h" ; CC1Nc2nc([C@H]3CCO[C@@H](c4cnn(C5CC5)c4)C3)nc(C3CC4(C3)CC(F)(F)C4)c2NC1C ; "MnO2, DCM,25° C., 12 h" ; Cc1nc2nc([C@H]3CCO[C@@H](c4cnn(C5CC5)c4)C3)nc(C3CC4(C3)CC(F)(F)C4)c2nc1C]

Step 1: Iodine (0.050 eq, 12 mg, 0.0474 mmol) in THF (0.5 mL) was added to a suspension of Mg (1.27 eq, 29 mg, 1.20 mmol) in dry THF (4 mL) under N₂ atmosphere, 6-bromo-2,2-difluoro-spiro[3.3]heptane (1.00 eq, 200 mg, 0.948 mmol) was added at 25° C., heated until the yellow solution turned into colorless solution, and then stirred at 25° C. for 1 h, a milky suspension was formed. ZnCl₂ (1.00 eq, 1.9 mL, 0.948 mmol) was added dropwise and the mixture was stirred for 30 minutes. A white precipitate formed. The reaction mixture was used directly with syringe.

Step 2: A sealed bottle under N₂ was charged with 2,4-dichloro-6,7-dimethyl-pteridine (1.00 eq, 50 mg, 0.218 mmol) and PdCl₂(Amphos) (0.0500 eq, 7.7 mg, 0.0109 mmol) and THF (3 mL) atmosphere and purged with N₂ three times, chloro-(2,2-difluorospiro[3.3]heptan-6-yl)zinc (4.51 eq, 228 mg, 0.985 mmol) was added dropwise to the reaction solution at 25° C., then warmed to 45° C. and stirred for 1 hr. The reaction solution was changed from yellow to dark brown, LCMS showed 47% of SM remained and 42% of desired mass was detected, the reaction solution was poured into H₂O, extracted with EtOAc, dried over Na₂SO₄ and evaporated under reduced pressure to give the residue, which was then purified with Flash column (PE:EA=3:1, Rf=0.4) and evaporated to give 2-chloro-4-(2,2-difluorospiro[3.3]heptan-6-yl)-6,7-dimethyl-pteridine (55 mg, 0.169 mmol, 77.59% yield) as yellow solid. LCMS (M+H)⁺=325.1; Retention time=0.871 min. [M+H]⁺=325.1; Retention time=0.871 min.

Step 3: To a solution of 2-chloro-4-(2,2-difluorospiro[3.3]heptan-6-yl)-6,7-dimethyl-pteridine (1.00 eq, 55 mg, 0.169 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 59 mg, 0.186 mmol) and K₂CO₃ (2.00 eq, 28 mg, 0.339 mmol) in 1,4-Dioxane (2 mL) and Water (0.4000 mL), [1,1′-Bis(diphenylphosphino)ferrocene]dichloropalladium(II) (0.0800 eq, 9.9 mg, 0.0135 mmol) was added and purged with N₂ for 3 times, the reaction solution was stirred at 100° C. for 2 hrs, LCMS showed the reactant was consumed and 36% of desired mass was detected. the reaction solution was poured into H₂O, extracted with EtOAc, dried over Na₂SO₄ and evaporated under reduced pressure to give the residue, which was then purified with Prep-TLC (pure EA, Rf=0.4) to give 2-[(6R)-6-(1-cyclopropylpyrazol-4-yl)-3,6-dihydro-2H-pyran-4-yl]-4-(2,2-difluorospiro[3.3]heptan-6-yl)-6,7-dimethyl-pteridine (32 mg, 0.0669 mmol, 39.48% yield) as yellow solid. [M+H]⁺=479.2; Retention time=0.967 min.

Step 4: To a solution of 2-[(6R)-6-(1-cyclopropylpyrazol-4-yl)-3,6-dihydro-2H-pyran-4-yl]-4-(2,2-difluorospiro[3.3]heptan-6-yl)-6,7-dimethyl-pteridine (1.00 eq, 32 mg, 0.0669 mmol) in Ethanol (3 mL) was added PtO₂ (1.00 eq, 15 mg, 0.0669 mmol) under N₂ atmosphere. The mixture was purged with H₂ for 3 times, then the mixture was stirred under H₂ atmosphere (15 psi) at 25° C. for 2 h. LCMS showed the reactant was consumed and 100% of desired mass was detected, the reaction solution was filtered through celatom and evaporated under reduced pressure to give 2-[(2R)-2-(1-cyclopropylpyrazol-4-yl)tetrahydropyran-4-yl]-4-(2,2-difluorospiro[3.3]heptan-6-yl)-6,7-dimethyl-5,6,7,8-tetrahydropteridine (32 mg, 0.0660 mmol, 98.75% yield) as yellow gum. [M+H]⁺=485.4; Retention time=0.723 min.

Step 5: To a solution 2-[(2R)-2-(1-cyclopropylpyrazol-4-yl)tetrahydropyran-4-yl]-4-(2,2-difluorospiro[3.3]heptan-6-yl)-6,7-dimethyl-5,6,7,8-tetrahydropteridine (1.00 eq, 32 mg, 0.0660 mmol) in DCM (4 mL) was added MnO₂ (15.0 eq, 86 mg, 0.991 mmol) and stirred at 25° C. for 12 h. LCMS showed the reactant was consumed and 100% of desired mass was detected, the reaction solution was filtered through celatom and evaporated under reduced pressure to give the residue, which was then purified with Prep-HPLC (FA) and lyophilized to give 2-[(2R)-2-(1-cyclopropylpyrazol-4-yl)tetrahydropyran-4-yl]-4-(2,2-difluorospiro[3.3]heptan-6-yl)-6,7-dimethyl-pteridine (6.1 mg, 0.0127 mmol, 19.19% yield) as off-white solid. [M+H]⁺=481.2; purity=100% (220 nm). Retention time=0.957 min. 1H NMR (400 MHz, CDCl₃) δ=7.51 (s, 2H), 4.75 (t, J=8.4 Hz, 1H), 4.57 (dd, J=1.8, 11.4 Hz, 1H), 4.31-4.23 (m, 1H), 3.87-3.76 (m, 1H), 3.61-3.54 (m, 1H), 3.50-3.41 (m, 1H), 2.79 (d, J=18.1 Hz, 7H), 2.74-2.56 (m, 7H), 2.41 (br d, J=13.3 Hz, 1H), 2.22-2.12 (m, 3H), 1.10 (br d, J=2.7 Hz, 2H), 1.00 (dd, J=2.1, 7.3 Hz, 2H), ee. 89%.

### Synthesis of Compounds I-1585

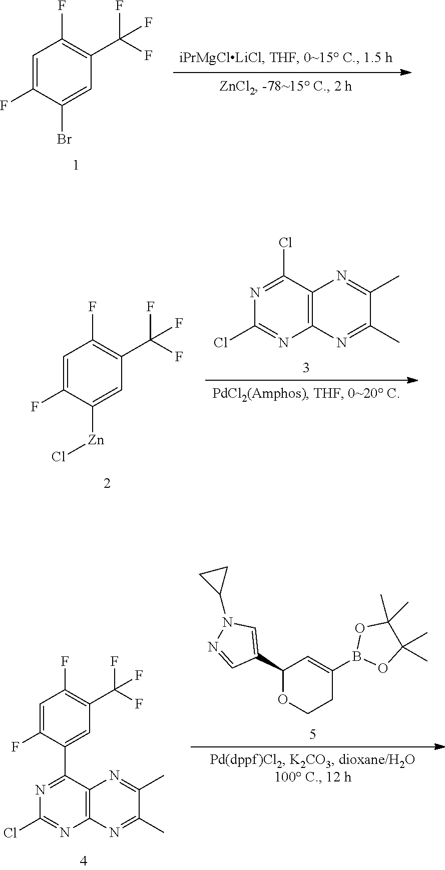

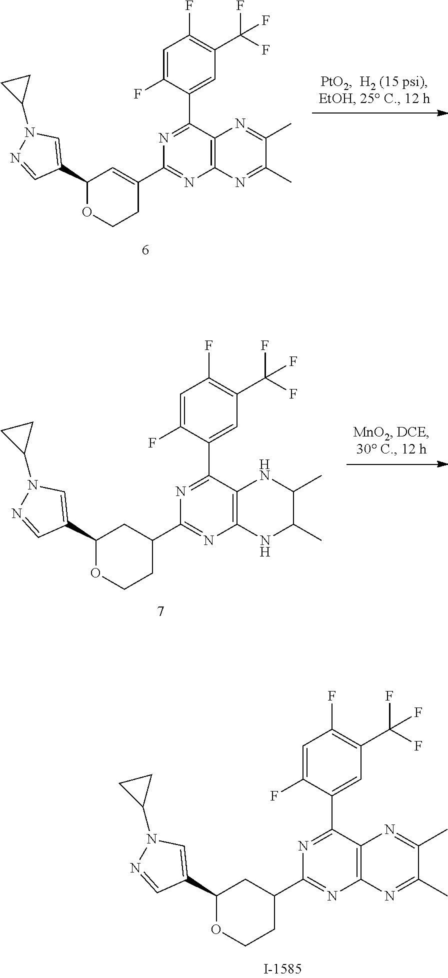 [SMILES: Cc1nc2nc(C3=C[C@H](c4cnn(C5CC5)c4)OCC3)nc(-c3cc(C(F)(F)F)c(F)cc3F)c2nc1C ; "PtO2,  H2 (15 psi),EtOH, 25° C., 12 h" ; CC1Nc2nc(C3CCO[C@@H](c4cnn(C5CC5)c4)C3)nc(-c3cc(C(F)(F)F)c(F)cc3F)c2NC1C ; "MnO2, DCE,30° C., 12 h" ; Cc1nc2nc(C3CCO[C@@H](c4cnn(C5CC5)c4)C3)nc(-c3cc(C(F)(F)F)c(F)cc3F)c2nc1C]

Step 1: To a solution of 1-bromo-2,4-difluoro-5-(trifluoromethyl)benzene (1.00 eq, 400 mg, 1.53 mmol) in THF (4 mL) was added iPrMgCl·LiCl (1.14 eq, 1.3 mL, 1.74 mmol) at 0° C. under N₂. The mixture was stirred at 15° C. for 1.5 h. LCMS showed that the starting material was consumed completely. ZnCl₂ (1.22 eq, 3.8 mL, 1.88 mmol) was added at −78° C. under N₂ and the mixture was stirred at 15° C. for 2 h. The reaction mixture was used directly for the next step ((2,4-difluoro-5-(trifluoromethyl)phenyl)zinc(II) chloride in THF (9.1 mL), 0.17 M).

Step 2: A sealed bottle under N₂ atmosphere was charged with 2,4-dichloro-6,7-dimethylpteridine (1.00 eq, 100 mg, 0.437 mmol) and PdCl₂(Amphos) (0.0500 eq, 15 mg, 0.0218 mmol) and THF (2 mL) and purged with N₂ for three times. Then cooled to 0° C., (2,4-difluoro-5-(trifluoromethyl)phenyl)zinc(II) chloride (1.50 eq, 3.9 mL, 0.655 mmol) was added dropwise to the reaction solution at 0° C., then warmed to 20° C. and stirred for 1 hr. The reaction solution was changed from orange to dark purple. LCMS showed the starting material remained and the desired product was detected (23%, Rt: 0.809 min; [M+H]⁺=375.2 at 220 nm). Then chloro-[2,4-difluoro-5-(trifluoromethyl)phenyl]zinc (1.50 eq, 3.9 mL, 0.655 mmol) was added at 0° C. and the mixture was stirred at 20° C. for 12 h. LCMS showed the starting material remained and the desired product was detected (33%, Rt: 0.913 min; [M+H]⁺=375.0 at 220 nm). The reaction solution was quenched with saturated NH₄Cl solution (20 mL), then extracted with EtOAc (30 mL×3) and evaporated under reduced pressure to give the residue, which was then purified with reversed-phase chromatography (0.1% FA) and lyophilized to give 2-chloro-4-(2,4-difluoro-5-(trifluoromethyl)phenyl)-6,7-dimethylpteridine (110 mg, 0.261 mmol, 59.85% yield) as yellow solid, checked by LCMS [M+H]⁺=375.3; purity=89% (220 nm). Retention time=0.811 min. ¹H NMR (400 MHz, DMSO-d₆) δ=8.22 (t, J=7.6 Hz, 1H), 7.95 (t, J=10.5 Hz, 1H), 2.81 (s, 3H), 2.70-2.66 (m, 3H).

Step 3: To a solution of (R)-1-cyclopropyl-4-(4-(4,4,5,5-tetramethyl-1,3,2-dioxaborolan-2-yl)-5,6-dihydro-2H-pyran-2-yl)-1H-pyrazole (1.10 eq, 102 mg, 0.323 mmol), 2-chloro-4-(2,4-difluoro-5-(trifluoromethyl)phenyl)-6,7-dimethylpteridine (1.00 eq, 110 mg, 0.294 mmol) and K₂CO₃ (3.00 eq, 74 mg, 0.881 mmol) in 1,4-Dioxane (5 mL) and water (0.5 mL) was added Pd(dppf)Cl₂ (0.0909 eq, 20 mg, 0.0267 mmol) at 20° C. The mixture was stirred at 100° C. for 12 h. LCMS showed that the starting material was consumed completely and the desired mass was major (39%, Rt: 0.801 min; [M+H]⁺=529.1 at 220 nm). The mixture was concentrated under reduced pressure to get the crude residue. The residue was purified via column chromatography on silica gel (PE/EtOAc=1/0 to 0/1; PE/EtOAc=0/1, the desired product R_{f}=0.4 at 254 nm) to give (R)-2-(6-(1-cyclopropyl-1H-pyrazol-4-yl)-3,6-dihydro-2H-pyran-4-yl)-4-(2,4-difluoro-5-(trifluoromethyl)phenyl)-6,7-dimethylpteridine (90 mg, 0.170 mmol, 58.01% yield) as orange solid, checked by LCMS [M+H]⁺=529.3; purity=90% (220 nm). Retention time=0.989 min. ¹H NMR (400 MHz, CDCl₃) δ=7.70-7.64 (m, 1H), 7.53 (d, J=12.0 Hz, 2H), 7.15 (t, J=9.6 Hz, 1H), 5.52-5.44 (m, 1H), 4.19-4.05 (m, 2H), 3.95 (ddd, J=5.1, 6.8, 11.7 Hz, 1H), 3.63-3.51 (m, 1H), 3.06-2.94 (m, 2H), 2.87 (s, 3H), 2.76-2.70 (m, 3H), 1.14-1.08 (m, 2H), 1.05-0.94 (m, 2H).

Step 4: To a solution of (R)-2-(6-(1-cyclopropyl-1H-pyrazol-4-yl)-3,6-dihydro-2H-pyran-4-yl)-4-(2,4-difluoro-5-(trifluoromethyl)phenyl)-6,7-dimethylpteridine (1.00 eq, 90 mg, 0.170 mmol) in EtOH (4 mL) was added PtO₂ (0.507 eq, 20 mg, 0.0864 mmol) under N₂ atmosphere. The mixture was purged with H₂ 3 times, then the mixture was stirred at 30° C. under H₂ (15 psi) for 12 h. LCMS showed that the starting material was consumed completely and the desired mass was detected (87%, Rt: 0.626 min; [M+H]⁺=535.5 at 220 nm). The reaction mixture was filtered and the filtrated was concentrated under reduced pressure to give the crude product 2-((2R)-2-(1-cyclopropyl-1H-pyrazol-4-yl)tetrahydro-2H-pyran-4-yl)-4-(2,4-difluoro-5-(trifluoromethyl)phenyl)-6,7-dimethyl-5,6,7,8-tetrahydropteridine (90 mg, 0.168 mmol, 98.87% yield) as yellow oil, checked by LCMS which was used directly for the next step. LCMS: [M+H]⁺=535.2; purity=60% (220 nm). Retention time=0.732 min.

Step 5: To a solution of 2-((2R)-2-(1-cyclopropyl-1H-pyrazol-4-yl)tetrahydro-2H-pyran-4-yl)-4-(2,4-difluoro-5-(trifluoromethyl)phenyl)-6,7-dimethyl-5,6,7,8-tetrahydropteridine (1.00 eq, 90 mg, 0.168 mmol) in DCE (4 mL) was added MnO₂ (20.0 eq, 293 mg, 3.37 mmol) at 20° C. and stirred at 30° C. for 12 h. LCMS showed that the starting material remained and the desired mass was detected (20%, Rt: 0.810 min; [M+H]⁺=531.5 at 220 nm). The reaction mixture was filtered and the filtrate was added MnO₂ (20.0 eq, 293 mg, 3.37 mmol). The reaction mixture was stirred at 30° C. for 12 h. LCMS showed that the desired mass was major (87%, Rt: 0.980 min; [M+H]⁺=531.2 at 220 nm). The reaction mixture was filtered and the filtrate was concentrated under reduced pressure to give a residue. The residue was purified by prep-HPLC (column: Phenomenex luna C18 150×25 mm×10 um; mobile phase: [water (FA)-ACN]; B %: 46%-76%, 12 min) and lyophilized to give 2-((2R)-2-(1-cyclopropyl-1H-pyrazol-4-yl)tetrahydro-2H-pyran-4-yl)-4-(2,4-difluoro-5-(trifluoromethyl)phenyl)-6,7-dimethylpteridinee (15 mg, 0.0285 mmol, 16.91% yield) as yellow solid (racemate): LCMS: [M+H]⁺=531.2; purity=98.7% (220 nm). Retention time=0.998 min. ¹H NMR (400 MHz, CDCl₃) δ=8.06 (t, J=7.4 Hz, 1H), 7.51 (d, J=1.1 Hz, 2H), 7.16 (t, J=9.6 Hz, 1H), 4.57 (dd, J=1.6, 11.4 Hz, 1H), 4.33-4.22 (m, 1H), 3.89-3.74 (m, 1H), 3.63-3.49 (m, 2H), 2.87 (s, 3H), 2.74 (s, 3H), 2.44 (br d, J=13.4 Hz, 1H), 2.28-2.16 (m, 3H), 1.13-1.05 (m, 2H), 1.04-0.95 (m, 2H). EE, 86%.

### Synthesis of I-1588

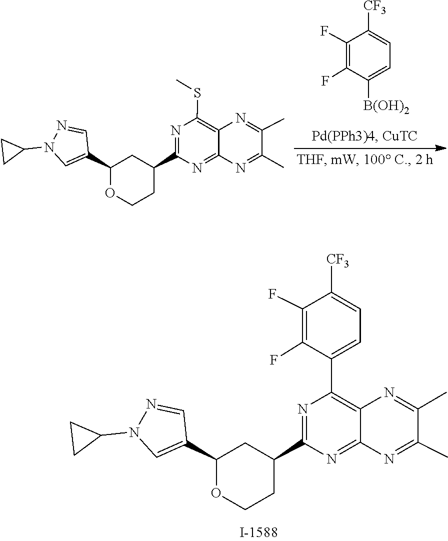 [SMILES: CSc1nc([C@H]2CCO[C@@H](c3cnn(C4CC4)c3)C2)nc2nc(C)c(C)nc12 ; OB(O)c1ccc(C(F)(F)F)c(F)c1F ; Cc1nc2nc([C@H]3CCO[C@@H](c4cnn(C5CC5)c4)C3)nc(-c3ccc(C(F)(F)F)c(F)c3F)c2nc1C]

In a flame dried 10 mL microwave vial under N₂ was charged with 6,7-dimethyl-4-methylsulfanyl-2-[(2R,4S)-2-(1-cyclopropylpyrazol-4-yl)tetrahydropyran-4-yl]pteridine (1.00 eq, 100 mg, 0.25 mmol), [2,3-difluoro-4-(trifluoromethyl)phenyl]boronic acid (1.50 eq, 85 mg, 0.38 mmol) and THF (5 mL). The reaction mixture was degassed under Argon for 5 min followed by the addition of Pd(PPh₃)₄ (0.30 eq, 87 mg, 0.08 mmol) and copper(I) thiophene-2-carboxylate (CuTC) (2.00 eq, 96 mg, 0.51 mmol). The reaction mixture was purged with N₂, the reaction vial was sealed with an aluminum cap with septa, and the reaction mixture was irradiated under constant microwave for 2 h with the reaction temperature controlled at 100° C. The mixture was filtered through a pad of Celite, washed with DCM (2×10 mL), concentrated, and purified by C18 flash chromatography (30 g Biotage C18 column) using a gradient from 10% to 60% ACN in water. The desired fractions were evaporated under reduced pressure to afford the desired analog as a mixture of diastereoisomers. The desired cis diastereoisomer was isolated by Prep-HPLC purification (Gemini® 5 um NX-C18 110 Å, 100×30 mm) using aqueous 10 mM ammonium formate and ACN (60-80%) to get 4-[2,3-difluoro-4-(trifluoromethyl)phenyl]-6,7-dimethyl-2-[(2R,4S)-2-(1-cyclopropyl pyrazol-4-yl)tetrahydropyran-4-yl]pteridine (70 mg, 0.132 mmol, 52% yield) as an off-white solid. ESI-MS (m/z+): 531.3 [M+H]. ¹H NMR (CHCl₃-d, 400 MHz): ^{6H} 7.53-7.58 (2H, m), 7.47 (2H, t, J=2.2 Hz), 4.54 (1H, dd, J=11.4, 2.1 Hz), 4.23-4.27 (1H, m), 3.75-3.82 (1H, m), 3.50-3.56 (2H, m), 2.85 (3H, s), 2.73 (3H, s), 2.39-2.44 (1H, m), 2.13-2.22 (3H, m), 1.05-1.09 (2H, m), 0.94-0.99 (2H, m).

### Synthesis of Compounds I-1593

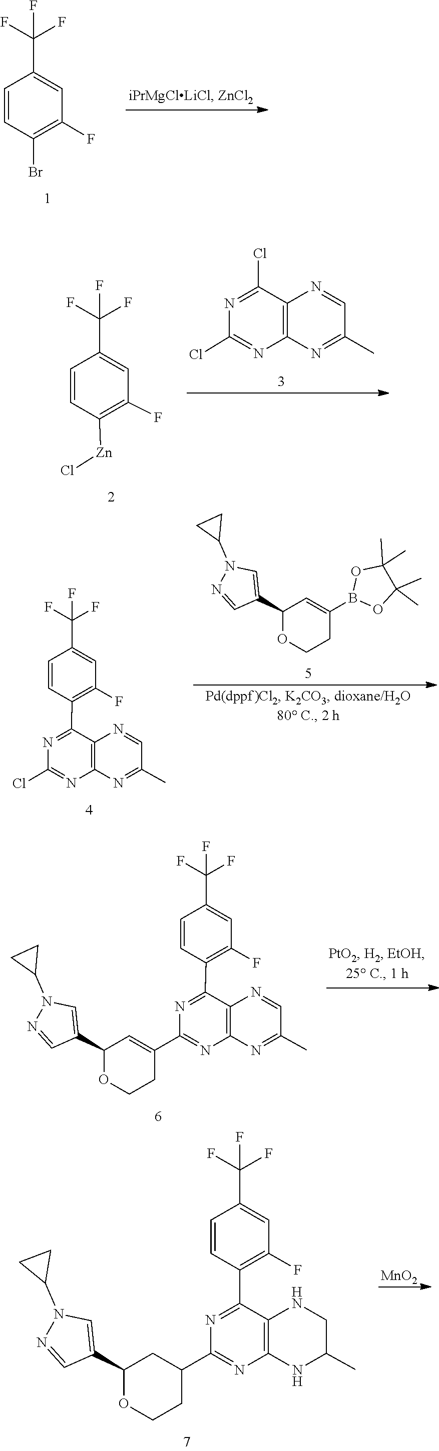

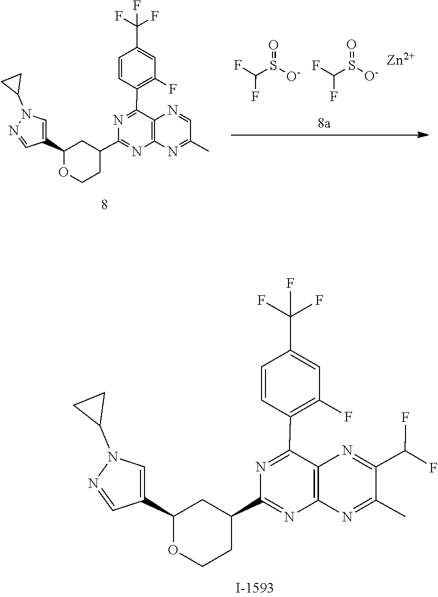 [SMILES: Cc1cnc2c(-c3ccc(C(F)(F)F)cc3F)nc(C3CCO[C@@H](c4cnn(C5CC5)c4)C3)nc2n1 ; O=S([O-])C(F)F ; O=S([O-])C(F)F ; [Zn+2] ; Cc1nc2nc([C@H]3CCO[C@@H](c4cnn(C5CC5)c4)C3)nc(-c3ccc(C(F)(F)F)cc3F)c2nc1C(F)F]

Step 1: To a solution of 1-bromo-2-fluoro-4-(trifluoromethyl)benzene (1.00 eq, 3000 mg, 12.3 mmol) in THE (30 mL) was added iPrMgCl—LiCl (1.11 eq, 11 mL, 13.7 mmol) at 0° C. under N₂ atmosphere. The mixture was stirred at 15° C. for 1.5 h. ZnCl₂ (1.21 eq, 30 mL, 15.0 mmol) was added at −78° C. under N₂ atmosphere and the mixture was stirred at 15° C. for 1 h. The reaction mixture was used directly for the next step.

Step 2: A sealed bottle under N₂ atmosphere was charged with 2,4-dichloro-7-methyl-pteridine (1.00 eq, 900 mg, 4.19 mmol) and PdCl₂(Amphos) (0.0500 eq, 148 mg, 0.209 mmol) and THF (9 mL) and purged with N₂ for three times, then cooled to 0° C., chloro-[2-fluoro-4-(trifluoromethyl)phenyl]zinc (1.00 eq, 25 mL, 4.19 mmol) was added dropwise to the reaction solution at 0° C., then warmed to 25° C. and stirred for 1 h. The reaction solution was changed from orange to dark purple, LCMS showed 57% of desired product was detected (57%, Rt=0.937 min; [M+H]⁺=343.1 at 220 nm).The reaction solution was quenched with saturated NH₄Cl solution (100 mL), extracted with EtOAc (150 mL*3) and evaporated under reduced pressure to give the residue, which was then purified with Flash column (PE:EA=0 to 40%, PE:EA=3: 1, Rf=0.5) and dried in vacuo to give 2-chloro-4-[2-fluoro-4-(trifluoromethyl)phenyl]-7-methyl-pteridine (900 mg, 2.44 mmol, 58.36% yield) as red solid, checked by LCMS [M+H]⁺=343.1; purity=93% (220 nm). Retention time=0.920 min. 1H NMR (400 MHz, CDCl3) δ=8.88 (s, 1H), 7.90 (t, J=7.3 Hz, 1H), 7.66 (d, J=8.1 Hz, 1H), 7.55 (d, J=9.4 Hz, 1H), 2.94 (s, 3H).

Step 3: 2-[(6R)-6-(1-cyclopropylpyrazol-4-yl)-3,6-dihydro-2H-pyran-4-yl]-4-[2-fluoro-4-(trifluoromethyl)phenyl]-7-methyl-pteridine. To a solution of 2-chloro-4-[2-fluoro-4-(trifluoromethyl)phenyl]-7-methyl-pteridine (1.00 eq, 900 mg, 2.63 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.15 eq, 955 mg, 3.02 mmol) and K₂CO₃ (3.00 eq, 662 mg, 7.88 mmol) in 1,4-Dioxane (40 mL) and Water (4 mL) was added Pd(dppf)Cl₂ DCM (0.120 eq, 231 mg, 0.315 mmol). The reaction mixture was stirred at 80° C. for 12 hours under N₂ atmosphere. LCMS showed the starting material was consumed completely and one major peak with desired product was detected (67%, Rt=0.971 min; [M+H]⁺=497.3 at 220 nm). The mixture was poured into 50 mL H₂O, extracted with EA (100 mL*3), the combine organic layers was dried over anhydrous Na₂SO₄, filtered and concentrated under reduced pressure to give the residue. The residue was purified by silica column chromatography (PE:EA=1:0 to 1:1, PE:EA=1:1, the desired product Rf=0.5) to give the 2-[(6R)-6-(1-cyclopropylpyrazol-4-yl)-3,6-dihydro-2H-pyran-4-yl]-4-[2-fluoro-4-(trifluoromethyl)phenyl]-7-methyl-pteridine (950 mg, 1.91 mmol, 72.86% yield) as red solid. [M+H]⁺=497.3; purity=87% (220 nm). Retention time=0.962 min. 1H NMR (400 MHz, CDCl3) δ=8.79 (s, 1H), 7.88 (t, J=7.3 Hz, 1H), 7.74-7.71 (m, 1H), 7.64 (d, J=8.0 Hz, 1H), 7.57-7.49 (m, 3H), 5.47 (q, J=2.5 Hz, 1H), 4.16-4.12 (m, 1H), 3.95 (ddd, J=5.0, 6.9, 11.6 Hz, 1H), 3.57 (tt, J=3.8, 7.3 Hz, 1H), 3.03-2.95 (m, 2H), 2.91 (s, 3H), 1.15-1.08 (m, 2H), 1.03-0.97 (m, 2H).

Step 4: To a solution of 2-[(6R)-6-(1-cyclopropylpyrazol-4-yl)-3,6-dihydro-2H-pyran-4-yl]-4-[2-fluoro-4-(trifluoromethyl)phenyl]-7-methyl-pteridine (1.00 eq, 800 mg, 1.61 mmol) in Ethanol (20 mL) was added Pd/C (0.937 eq, 160 mg, 1.51 mmol) under N₂ atmosphere. The mixture was purged with H₂ (15 psi) three times, then the mixture was stirred at 30° C. for 12 hours under H₂ (15 psi) atmosphere. LCMS showed the starting material was consumed completely and one major peak with the desired product was detected (82%, Rt=0.808 min; [M+H]⁺=503.2 at 220 nm, contain intermediated product).The mixture was stirred at 30° C. for another 12 hours. LCMS showed the starting material was consumed completely and one major peak with the desired product was detected (92%, Rt=0.808 min; [M+H]⁺=503.2 at 220 nm). The reaction mixture was filtered and concentrated under reduced pressure to give crude 2-[(2R)-2-(1-cyclopropylpyrazol-4-yl)tetrahydropyran-4-yl]-4-[2-fluoro-4-(trifluoromethyl)phenyl]-7-methyl-5,6,7,8-tetrahydropteridine (850 mg, 1.69 mmol, 104.97% yield) as dark yellow solid and the residue was used to the next step directly.

Step 5: To a solution of 2-[(2R)-2-(1-cyclopropylpyrazol-4-yl)tetrahydropyran-4-yl]-4-[2-fluoro-4-(trifluoromethyl)phenyl]-7-methyl-5,6,7,8-tetrahydropteridine (1.00 eq, 850 mg, 1.69 mmol) in dry DCE (50 mL) was added MnO₂ (20.0 eq, 2941 mg, 33.8 mmol). The mixture was stirred at 30° C. for 12 h. LCMS showed 27% of starting material was remained, 29% of desired product was detected (29%, Rt=0.968 min, [M+H]⁺=499.3, ESI⁺) and anew peak with MS=SM+16 was detected. The mixture was filtered and the filtered cake was washed with EA (20 mL*3), the combine organic layers was concentrated under reduced pressure to give the residue. The residue was redissolved in dry DCM (50 mL), MnO₂ (20.0 eq, 2941 mg, 33.8 mmol) was added to the mixture and then the mixture was stirred at 30° C. for another 12 h. LCMS showed the starting material consumed completely and 59% of desired product was detected (59%, Rt=0.968 min, [M+H]⁺=499.3). The mixture was filtered and the filter cake was washed with EA (50 mL*3), the combine organic layers was concentrated under reduced pressure to give the residue. The residue was purified by column silica chromatography (PE:EA=1:0 to 1:2, PE:EA=0:1, the desired product Rf=0.5) to give the 2-[(2R)-2-(1-cyclopropylpyrazol-4-yl)tetrahydropyran-4-yl]-4-[2-fluoro-4-(trifluoromethyl)phenyl]-7-methyl-pteridine (320 mg, 0.642 mmol, 37.95% yield) as red oil, LCMS [M+H]⁺=499.3; purity=78% (220 nm). Retention time=0.934 min.

Step 6: To a solution of -[(2R,4S)-2-(1-cyclopropylpyrazol-4-yl)tetrahydropyran-4-yl]-4-[2-fluoro-4-(trifluoromethyl)phenyl]-7-methyl-pteridine (1.00 eq, 100 mg, 0.201 mmol) and zinc difluoromethanesulfinate (4.00 eq, 236 mg, 0.802 mmol) in DMSO (10 mL) at 25° C., tert-butylhydroperoxide (7.00 eq, 181 mg, 1.40 mmol) in DMSO (2 mL) was added with vigorous stirring and bubbled with N₂ for 30 seconds. The reaction solution was stirred at 30° C. for 4 hrs. LCMS showed 75% of starting material was remained and 15% of desired product was detected (15%, Rt=0.981 min, [M+H]⁺=549.3 at 220 nm). The reaction solution was stirred at 30° C. for another 12 hrs. LCMS showed the starting material was consumed completely and 40% of desired product was detected (40%, Rt=1.003 min, [M+H]⁺=549.3 at 220 nm). The combined mixture was quenched by 10 mL Na₂S₂O₃, then extracted with EA (20 mL*3), the combine organic layers was dried over anhydrous Na₂SO₄, filtered and concentrated under reduced pressure to give the residue. The residue was purified by Prep-TLC (PE:EA=0:1, the desired product Rf=0.3) to give a crude product. The crude product was purified again by Prep-HPLC (Phenomenex luna C18 150*25 mm*10 um, water (FA)-ACN) and lyophilized to give the 2-[(2R,4S)-2-(1-cyclopropylpyrazol-4-yl)tetrahydropyran-4-yl]-6-(difluoromethyl)-4-[2-fluoro-4-(trifluoromethyl)phenyl]-7-methyl-pteridine (3.4 mg, 0.00555 mmol, 2.76% yield) as a solid, LCMS [M+H]⁺=549.3; purity=98.4% (220 nm). Retention time=1.016 min. 1H NMR (400 MHz, CDCl3) δ=7.88 (t, J=7.3 Hz, 1H), 7.66 (d, J=8.1 Hz, 1H), 7.59-7.45 (m, 3H), 6.91-6.58 (m, 1H), 4.58 (dd, J=1.9, 11.4 Hz, 1H), 4.29 (td, J=3.1, 11.2 Hz, 1H), 3.90-3.78 (m, 1H), 3.67-3.51 (m, 2H), 3.08 (s, 3H), 2.47 (br d, J=13.1 Hz, 1H), 2.21 (s, 3H), 1.18-1.06 (m, 2H), 1.03-0.94 (m, 2H). SFC showed 100% ee, purity was 90% based on the result of 1H NMR and LCMS.

### Synthesis of Compounds I-1598 and I-1638

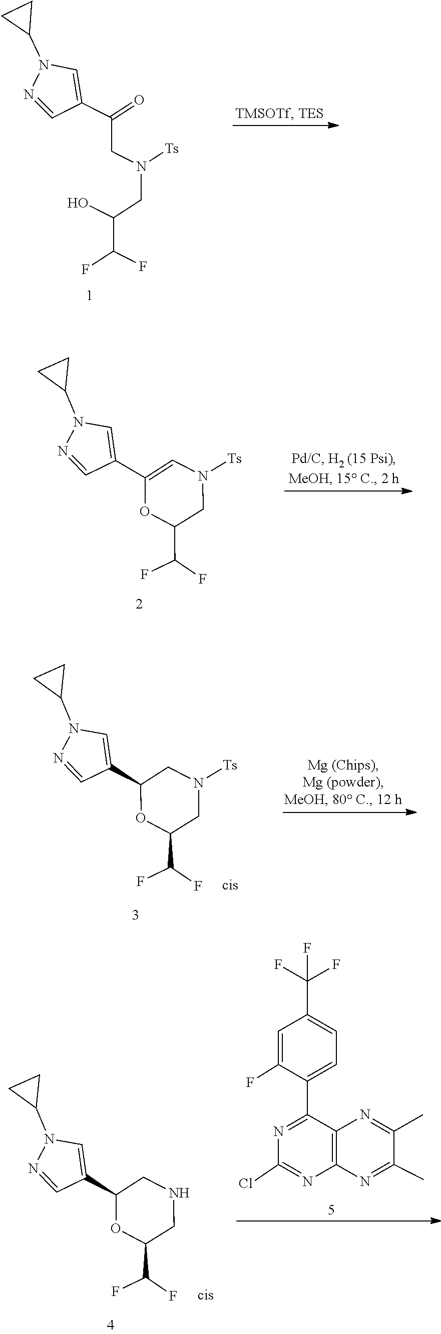 [SMILES: Cc1ccc(S(=O)(=O)N(CC(=O)c2cnn(C3CC3)c2)CC(O)C(F)F)cc1 ; Cc1ccc(S(=O)(=O)N2C=C(c3cnn(C4CC4)c3)OC(C(F)F)C2)cc1 ; Cc1ccc(S(=O)(=O)N2C[C@@H](C(F)F)O[C@@H](c3cnn(C4CC4)c3)C2)cc1 ; FC(F)[C@@H]1CNC[C@H](c2cnn(C3CC3)c2)O1 ; Cc1nc2nc(Cl)nc(-c3ccc(C(F)(F)F)cc3F)c2nc1C]

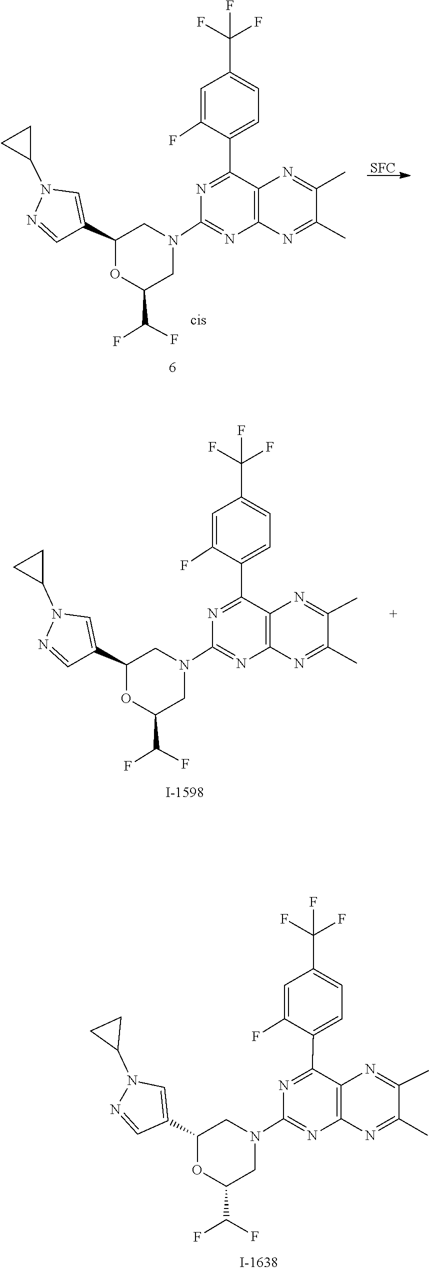

Step 1: To a solution of N-[2-(1-cyclopropylpyrazol-4-yl)-2-oxo-ethyl]-N-(3,3-difluoro-2-hydroxy-propyl)-4-methyl-benzenesulfonamide (1.00 eq, 300 mg, 0.726 mmol) and triethylsilane (19.0 eq, 2.2 mL, 13.8 mmol) in DCM (5 mL) was added TMSOTf (8.38 eq, 1.1 mL, 6.08 mmol) at 0° C. The mixture was stirred at 0° C. for 0.5 h. LCMS showed 37% of desired product was detected (37%, Rt=0.965 min; [M+H]⁺=396.0 at 220 nm). The mixture was quenched with sat. aq NaHCO₃ (50 mL), extracted with EtOAc (30 mL*3), the combined organic layers were washed with brine, dried over anhydrous Na₂SO₄, filtered and concentrated to give the 6-(1-cyclopropylpyrazol-4-yl)-2-(difluoromethyl)-4-(p-tolylsulfonyl)-2,3-dihydro-1,4-oxazine (250 mg, 0.499 mmol, 68.83% yield) as white solid, checked by LCMS and H NMR. [M+H]⁺=396.0; purity=79% (220 nm). Retention time=0.958 min. 1H NMR (400 MHz, CDCl3) δ=7.67 (d, J=8.3 Hz, 2H), 7.49 (d, J=12.0 Hz, 1H), 7.42-7.36 (m, 1H), 7.33 (d, J=8.2 Hz, 2H), 5.92-5.56 (m, 1H), 3.94 (br d, J=13.3 Hz, 1H), 3.59-3.51 (m, 1H), 3.50-3.42 (m, 1H), 3.24 (dd, J=8.6, 13.5 Hz, 1H), 2.47-2.42 (m, 3H), 2.04 (s, 2H), 1.12-1.06 (m, 2H), 1.01 (br dd, J=1.1, 6.2 Hz, 2H), 0.92 (s, 1H).

Step 2: To a solution of 6-(1-cyclopropylpyrazol-4-yl)-2-(difluoromethyl)-4-(p-tolylsulfonyl)-2,3-dihydro-1,4-oxazine (1.00 eq, 250 mg, 0.632 mmol) in methanol (5 mL) was added Pd/C (1.00 eq, 67 mg, 0.632 mmol) at 15° C. Then the reaction mixture was degassed with H₂ for 3 times. The reaction mixture was stirred at 15° C. for 2 h under H₂ atmosphere (15 psi). LCMS showed 32% of desired product was detected (32%, Rt=0.923 min; [M+H]⁺=398.2 at 220 nm). The reaction mixture was stirred at 30° C. for another 6 h under H₂ atmosphere (15 psi). LCMS showed 95% of desired product was detected (95%, Rt=0.939 min; [M+H]⁺=398.2 at 220 nm). The solution was filtered and the filter cake was washed with MeOH (30 mL*3), the combine organic layers was concentrated under reduced pressure to give the 2-(1-cyclopropylpyrazol-4-yl)-6-(difluoromethyl)-4-(p-tolylsulfonyl)morpholine (200 mg, 0.503 mmol, 79.59% yield) as colorless oil.

Step 3: To a colorless mixture of 2-(1-cyclopropylpyrazol-4-yl)-6-(difluoromethyl)-4-(p-tolylsulfonyl)morpholine (1.00 eq, 190 mg, 0.478 mmol) in methanol (2 mL) was added Mg (Chips) (10.0 eq, 115 mg, 4.78 mmol) and Mg (powder) (10.0 eq, 115 mg, 4.78 mmol). The mixture was stirred at 80° C. for 12 h under N₂ atmosphere. LCMS showed 93% of desired product was detected (93%, Rt=0.334 min; [M+H]⁺=244.3 at 220 nm). The reaction mixture was cooled to room temperature. The reaction mixture was filtered, the filtrate was concentrated under reduced pressure to give 2-(1-cyclopropylpyrazol-4-yl)-6-(difluoromethyl)morpholine (90 mg, 0.370 mmol, 77.39% yield) as white solid.

Step 4: To a solution of 2-(1-cyclopropylpyrazol-4-yl)-6-(difluoromethyl)morpholine (1.00 eq, 70 mg, 0.288 mmol) in DMSO (1.5 mL) was added 2-chloro-4-[2-fluoro-4-(trifluoromethyl)phenyl]-6,7-dimethyl-pteridine (1.00 eq, 103 mg, 0.288 mmol) and DIEA (3.00 eq, 0.15 mL, 0.863 mmol). The mixture was stirred at 100° C. for 1 h. LCMS showed 47% of desired product was detected (47%, Rt=1.012 min; [M+H]⁺=564.3 at 220 nm). The mixture was quenched by 60 mL H₂O, extracted with EA (30 mL*3), the combine organic layers was dried over anhydrous Na₂SO₄, filtered and concentrated under reduced pressure to give the residue. The residue was purified by prep-HPLC (flow: 25 mL/min; gradient: from 60-80% water (0.1% FA)-ACN over 10 min; column: Phenomenex luna C18 150*25 mm*5 um) and lyophilized to afford (2S,6S)-2-(1-cyclopropylpyrazol-4-yl)-6-(difluoromethyl)-4-[4-[2-fluoro-4-(trifluoromethyl)phenyl]-6,7-dimethyl-pteridin-2-yl]morpholine (42 mg, 0.0743 mmol, 25.80% yield) as a brown solid.

Step 5: The product was purified by SFC (Column: (S,S)Whelk-O1 100×4.6 mm I.D., 3.5 um Mobile phase: Phase A for CO₂ and Phase B for MeOH+ACN (0.05% DEA); Gradient elution: 40% MeOH+ACN (0.05% DEA) in CO₂; Flow rate: 3 mL/min; Detector: PDA, Column Temp: 35C; Back Pressure: 100 Bar and lyophilized to give the (2S,6S)-2-(1-cyclopropylpyrazol-4-yl)-6-(difluoromethyl)-4-[4-[2-fluoro-4-(trifluoromethyl)phenyl]-6,7-dimethyl-pteridin-2-yl]morpholine (12 mg, 0.0217 mmol, 30.77% yield) as yellow solid, (2R,6R)-2-(1-cyclopropylpyrazol-4-yl)-6-(difluoromethyl)-4-[4-[2-fluoro-4-(trifluoromethyl)phenyl]-6,7-dimethyl-pteridin-2-yl]morpholine (14 mg, 0.0250 mmol, 35.42% yield) as yellow solid, 564.3 [M+H]f; purity=100.0% (220 nm). Retention time=1.048 min. 1H NMR (400 MHz, CDCl3) δ=7.82 (t, J=7.1 Hz, 1H), 7.60 (d, J=8.3 Hz, 1H), 7.55 (s, 2H), 7.51 (br d, J=9.4 Hz, 1H), 6.03-5.70 (m, 1H), 5.15 (br dd, J=2.0, 10.7 Hz, 2H), 4.67 (dd, J=2.1, 10.9 Hz, 1H), 4.09-3.91 (m, 1H), 3.59 (tt, J=3.6, 7.3 Hz, 1H), 3.15 (ddd, J=8.3, 11.0, 13.4 Hz, 2H), 2.75 (s, 3H), 2.61 (s, 3H), 1.16-1.10 (m, 2H), 1.08-1.01 (m, 2H). SFC showed ee, 100%. [M+H]⁺ 564.3 purity=100% (220 nm). Retention time=1.042 min. 1H NMR (400 MHz, CDCl3) δ=7.82 (t, J=7.4 Hz, 1H), 7.60 (d, J=8.6 Hz, 1H), 7.55 (s, 2H), 7.51 (d, J=9.4 Hz, 1H), 6.04-5.69 (m, 1H), 5.23-5.06 (m, 2H), 4.71-4.61 (m, 1H), 4.07-3.91 (m, 1H), 3.60 (qd, J=3.6, 7.1 Hz, 1H), 3.22-3.09 (m, 2H), 2.75 (s, 3H), 2.61 (s, 3H), 1.26 (s, 1H), 1.18-1.10 (m, 2H), 1.07-0.99 (m, 2H). SFC showed ee >99%.

### Synthesis of Compounds I-1603

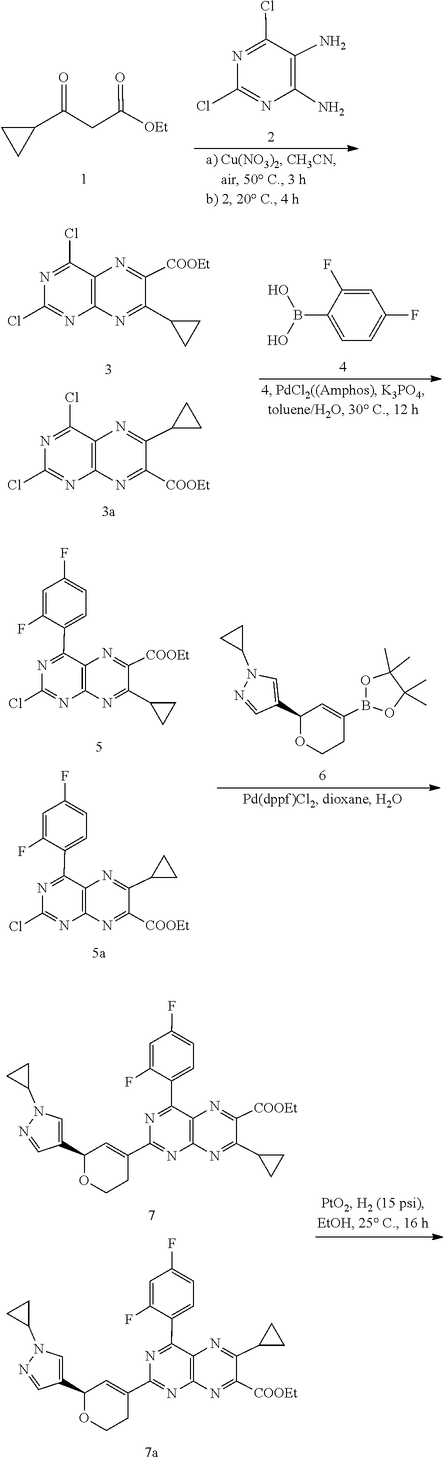

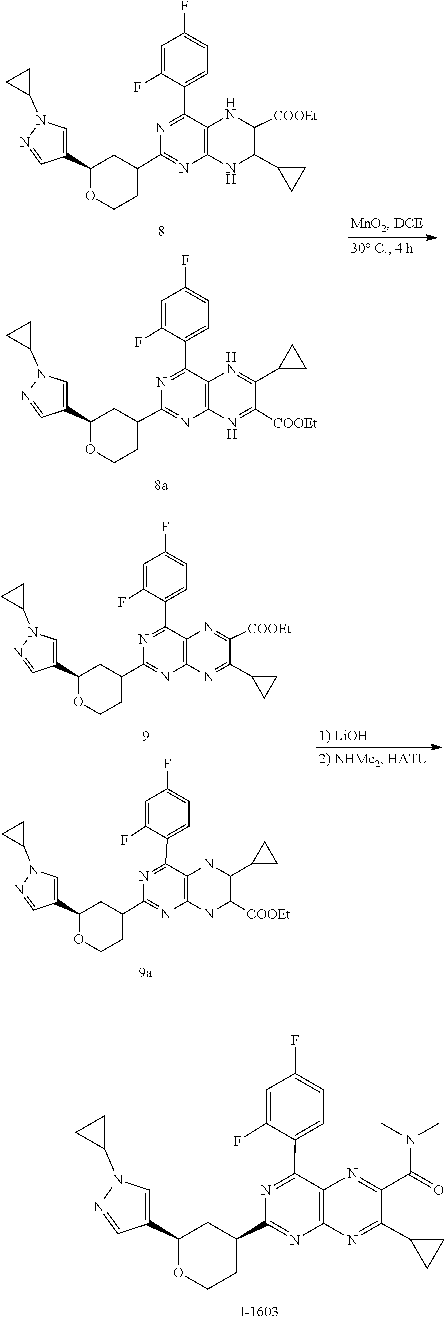 [SMILES: CCOC(=O)C1Nc2c(nc(C3CCO[C@@H](c4cnn(C5CC5)c4)C3)nc2-c2ccc(F)cc2F)NC1C1CC1 ; CCOC(=O)c1nc2nc(C3CCO[C@@H](c4cnn(C5CC5)c4)C3)nc(-c3ccc(F)cc3F)c2nc1C1CC1 ; CCOC(=O)c1nc2c(-c3ccc(F)cc3F)nc(C3CCO[C@@H](c4cnn(C5CC5)c4)C3)nc2nc1C1CC1 ; CCOC(=O)C1Nc2nc(C3CCO[C@@H](c4cnn(C5CC5)c4)C3)nc(-c3ccc(F)cc3F)c2NC1C1CC1 ; CN(C)C(=O)c1nc2c(-c3ccc(F)cc3F)nc([C@H]3CCO[C@@H](c4cnn(C5CC5)c4)C3)nc2nc1C1CC1]

Step 1: To a solution of ethyl 3-cyclopropyl-3-oxo-propanoate (1.60 eq, 3360 mg, 21.5 mmol) in MeCN (120 mL) was added Cu (NO₃)₂·3H₂O (0.356 eq, 1152 mg, 4.77 mmol) in one portion, then the mixture was stirred at 50° C. for 6 h under air, the mixture was cooled to 20° C., then 2,6-dichloropyrimidine-4,5-diamine (1.00 eq, 2400 mg, 13.4 mmol) was added by portions into the reaction mixture, the mixture was stirred at 20° C. for 4 h. LCMS showed raw material was consumed completely and the major peak showed MS (71%, Rt: 0.923 min; [M+H]⁺=313.0 at 220 nm). The mixture was filtered and then the filtrate was evaporated to give the residue, the residue was purified by column chromatography (hexane/ethyl acetate=0 to 50%, PE/EtOAc=2/1, the desired product Rf=0.3) to obtain crude ethyl 2,4-dichloro-7-cyclopropyl-pteridine-6-carboxylate (600 mg, 1.92 mmol, 14.29% yield) and ethyl 2,4-dichloro-6-cyclopropyl-pteridine-7-carboxylate (600 mg, 1.92 mmol, 14.29% yield) as a light-yellow solid, checked by LCMS [M+H]⁺=312.9; purity=91% (220 nm). Retention time=0.934 and 0.954 min.

Step 2: A sealed bottle under N₂ atmosphere was charged with ethyl 2,4-dichloro-7-cyclopropyl-pteridine-6-carboxylate (1.00 eq, 500 mg, 1.60 mmol), ethyl 2,4-dichloro-6-cyclopropyl-pteridine-7-carboxylate (1.00 eq, 500 mg, 1.60 mmol), (2,5-difluorophenyl)boronic acid (0.800 eq, 202 mg, 1.28 mmol), K₃PO₄ (3.00 eq, 1017 mg, 4.79 mmol) and PdCl₂(Amphos) (0.0500 eq, 57 mg, 0.0798 mmol) and purged with N₂ three times, then toluene (12 mL) and water (1.2 mL) was added in one portion at 15° C., then the mixture was stirred at 15° C. for 2 h and 30° C. for 10 h. LCMS showed raw material was consumed completely and the major peak showed MS (60%, Rt: 0.998 min; [M+H]⁺=391.0 at 220 nm). The reaction mixture was filtered and the filter cake was washed with EtOAc (30 mL), combined the solvent, water (50 mL) was added and then the organic phase was dried over Na₂SO₄, filtered and concentrated under reduced pressure evaporate to give the residue, the residue was purified by column chromatography (hexane/ethyl acetate=0 to 30%, PE/EtOAc=2/1, the desired product Rf=0.5) to obtain ethyl 2-chloro-7-cyclopropyl-4-(2,5-difluorophenyl)pteridine-6-carboxylate (380 mg, 0.654 mmol, 40.95% yield) and ethyl 2-chloro-6-cyclopropyl-4-(2,5-difluorophenyl)pteridine-7-carboxylate (380 mg, 0.654 mmol, 40.95% yield) as colorless oil. [M+H]⁺=391.0. Retention time=0.998 min.

Step 3: To a solution of 1-cyclopropyl-4-[(6R)-4-(4,4,5,5-tetramethyl-1,3,2-dioxaborolan-2-yl)-3,6-dihydro-2H-pyran-6-yl]pyrazole (2.00 eq, 210 mg, 0.665 mmol), Pd(dppf)Cl₂ DCM (0.200 eq, 49 mg, 0.0665 mmol) and ethyl 2-chloro-7-cyclopropyl-4-(2,4-difluorophenyl)pteridine-6-carboxylate (1.00 eq, 130 mg, 0.333 mmol), ethyl 2-chloro-6-cyclopropyl-4-(2,4-difluorophenyl)pteridine-7-carboxylate (1.00 eq, 130 mg, 0.333 mmol) in 1,4-Dioxane (8 mL) and Water (0.8 mL) was added K₂CO₃ (6.00 eq, 168 mg, 2.00 mmol) in one portion. Then the reaction mixture was stirred at 80° C. for 3 hours under N₂ atmosphere. LCMS showed the starting material was consumed completely and one major peak with desired product was detected (63.5%, Rt: 1.008 min; [M+H]⁺=545.2 at 220 nm). The mixture was poured into H₂O (80 mL), extracted with EtOAc (100 mL * 2), the combine organic layers was dried over anhydrous Na₂SO₄, filtered and concentrated under reduced pressure to give the residue. The residue was purified by Prep-TLC (PE/EtOAc=1/2, the desired product Rf=0.6) to give the ethyl 7-cyclopropyl-2-[(6R)-6-(1-cyclopropylpyrazol-4-yl)-3,6-dihydro-2H-pyran-4-yl]-4-(2,4-difluorophenyl)pteridine-6-carboxylate (120 mg, 0.220 mmol, 66.24% yield) as yellow solid and ethyl 6-cyclopropyl-2-[(6R)-6-(1-cyclopropylpyrazol-4-yl)-3,6-dihydro-2H-pyran-4-yl]-4-(2,4-difluorophenyl)pteridine-7-carboxylate (120 mg, 0.220 mmol, 66.24% yield) as yellow solid, checked by LCMS [M+H]⁺=545.2; purity=91.9% (220 nm). Retention time=0.869 min.

Step 4: To a solution of ethyl 7-cyclopropyl-2-[(6R)-6-(1-cyclopropylpyrazol-4-yl)-3,6-dihydro-2H-pyran-4-yl]-4-(2,4-difluorophenyl)pteridine-6-carboxylate (1.00 eq, 170 mg, 0.312 mmol) and ethyl 6-cyclopropyl-2-[(6R)-6-(1-cyclopropylpyrazol-4-yl)-3,6-dihydro-2H-pyran-4-yl]-4-(2,4-difluorophenyl)pteridine-7-carboxylate (1.00 eq, 170 mg, 0.312 mmol) in Ethanol (15 mL) was added PtO₂ (2.00 eq, 142 mg, 0.624 mmol) under N₂ atmosphere. The mixture was purged with H₂ (15 psi) 3 times, then the mixture was stirred at 15° C. for 12 hours under H₂ (15 psi) atmosphere. LCMS showed the starting material was consumed completely and desired product was detected (61.6%, Rt: 0.814 and 0.831 min; [M+H]⁺=551.1 at 220 nm). The suspension was filtered through a pad of celite and the filter cake was washed with MeOH (5 mL×3). The combined filtrates were concentrated to dryness to give ethyl 7-cyclopropyl-2-[(2R)-2-(1-cyclopropylpyrazol-4-yl)tetrahydropyran-4-yl]-4-(2,4-difluorophenyl)pteridine-6-carboxylate (160 mg, 0.293 mmol, 93.77% yield) and ethyl 6-cyclopropyl-2-[(2R)-2-(1-cyclopropylpyrazol-4-yl)tetrahydropyran-4-yl]-4-(2,4-difluorophenyl)pteridine-7-carboxylate (160 mg, 0.293 mmol, 93.77% yield) as a yellow gum. [M+H]⁺=551.1, purity=61.6% (220 nm). Retention time=0.814 and 0.831 min.

Step 5: To a solution of ethyl 7-cyclopropyl-2-[(6R)-6-(1-cyclopropylpyrazol-4-yl)-3,6-dihydro-2H-pyran-4-yl]-4-(2,4-difluorophenyl)pteridine-6-carboxylate (1.00 eq, 170 mg, 0.312 mmol) and ethyl 6-cyclopropyl-2-[(6R)-6-(1-cyclopropylpyrazol-4-yl)-3,6-dihydro-2H-pyran-4-yl]-4-(2,4-difluorophenyl)pteridine-7-carboxylate (1.00 eq, 170 mg, 0.312 mmol) in DCE (10 mL) was added MnO₂ (15.0 eq, 284 mg, 3.27 mmol) in one portion. The mixture was stirred at 30° C. for 4 hour. LCMS found major was desired MS, showed the starting material was consumed completely and desired product was detected, (90.7%, Rt: 0.997 min; [M+H]⁺=547.1 at 220 nm). The suspension was filtered through a pad of celite and the pad was washed with MeOH (5 mL×3). The combined filtrate was concentrated to dryness to give ethyl 7-cyclopropyl-2-[(2R)-2-(1-cyclopropylpyrazol-4-yl)tetrahydropyran-4-yl]-4-(2,4-difluorophenyl)pteridine-6-carboxylate (110 mg, 0.201 mmol, 92.34% yield) and ethyl 6-cyclopropyl-2-[(2R)-2-(1-cyclopropylpyrazol-4-yl)tetrahydropyran-4-yl]-4-(2,4-difluorophenyl)pteridine-7-carboxylate (110 mg, 0.201 mmol, 92.34% yield) as yellow solid, checked by LCMS [M+H]⁺=545.2; purity=91.7% (220 nm). Retention time=0.914 min.

Step 6: To a solution of ethyl 7-cyclopropyl-2-[(2R)-2-(1-cyclopropylpyrazol-4-yl)tetrahydropyran-4-yl]-4-(2,4-difluorophenyl)pteridine-6-carboxylate (1.00 eq, 75 mg, 0.137 mmol) and ethyl 6-cyclopropyl-2-[(2R)-2-(1-cyclopropylpyrazol-4-yl)tetrahydropyran-4-yl]-4-(2,4-difluorophenyl)pteridine-7-carboxylate (1.00 eq, 75 mg, 0.137 mmol) in THF (3 mL) and water (2 mL) was added NaOH (31.2 eq, 373 mg, 4.29 mmol) in one portion. Then the mixture was stirred at 15° C. for 12 h. LCMS showed the starting material was consumed completely and desired product was detected (89.9%, Rt: 0.887 min; [M+H]⁺=519.2 at 220 nm). The mixture was purified by reversed-phase column (C18 150*40 mm*15 um, 0.1% FA) and the solvent was lyophilized to give 7-cyclopropyl-2-[(2R)-2-(1-cyclopropylpyrazol-4-yl)tetrahydropyran-4-yl]-4-(2,4-difluorophenyl)pteridine-6-carboxylic acid (50 mg, 0.0964 mmol, 70.27% yield) and 6-cyclopropyl-2-[(2R)-2-(1-cyclopropylpyrazol-4-yl)tetrahydropyran-4-yl]-4-(2,4-difluorophenyl)pteridine-7-carboxylate (50 mg, 0.0964 mmol, 70.27% yield) as yellow solid, checked by LCMS [M+H]⁺=519.2; purity=88.7% (220 nm). Retention time=0.808 min.

Step 7: To a solution of 7-cyclopropyl-2-[(2R)-2-(1-cyclopropylpyrazol-4-yl)tetrahydropyran-4-yl]-4-(2,4-difluorophenyl)pteridine-6-carboxylic acid (1.00 eq, 35 mg, 0.0675 mmol) and 6-cyclopropyl-2-[(2R)-2-(1-cyclopropylpyrazol-4-yl)tetrahydropyran-4-yl]-4-(2,4-difluorophenyl)pteridine-7-carboxylic acid (0.100 eq, 3.5 mg, 0.00675 mmol) in DMF (1 mL) was added HATU (1.50 eq, 38 mg, 0.101 mmol) and DIEA (3.00 eq, 0.035 mL, 0.203 mmol) in one portion. Then the mixture was stirred at 10° C. for 10 min. Then dimethylamine hydrochloride (4.00 eq, 22 mg, 0.270 mmol) was added in one portion, then the mixture was stirred at 10° C. for 2 h. LCMS showed the starting material was consumed completely and desired product was detected (61.7%, Rt: 0.921 min; [M+H]⁺=546.1 at 220 nm). The reaction mixture was purified by Prep-HPLC (C18 150*40 mm*15 um, 0.1% FA) and the solvent was lyophilized to give 7-cyclopropyl-2-[(2R)-2-(1-cyclopropylpyrazol-4-yl)tetrahydropyran-4-yl]-4-(2,4-difluorophenyl)-N,N-dimethyl-pteridine-6-carboxamide (31 mg, 0.0562 mmol, 83.26% yield) as yellow solid. 98.9% purity, Rt: 0.909 min; [M+H]⁺=546.2 at 220 nm. ¹H NMR (400 MHz, CDCl3, 297 K) Shift (ppm)=7.79-7.70 (m, 1H), 7.49 (s, 2H), 7.09 (dt, J=2.4, 8.2 Hz, 1H), 7.02-6.94 (m, 1H), 4.55 (dd, J=2.0, 11.2 Hz, 1H), 4.27 (br dd, J=3.0, 11.6 Hz, 1H), 3.80 (dt, J=1.8, 11.8 Hz, 1H), 3.62-3.51 (m, 2H), 3.22 (s, 3H), 3.00 (s, 3H), 2.35 (dt, J=4.2, 8.2 Hz, 2H), 2.27-2.16 (m, 2H), 2.10 (br dd, J=1.9, 13.3 Hz, 1H), 1.61 (br dd, J=3.2, 4.2 Hz, 2H), 1.33 (br dd, J=3.4, 7.8 Hz, 2H), 1.09 (br d, J=2.8 Hz, 2H), 1.03-0.95 (m, 2H). ¹⁹F NMR (376.5 MHz, CDCl3, 297 K) Shift (ppm) −105.72, -106.58. Chiral SFC, ee. 95%.

### Synthesis of Compounds I-1609

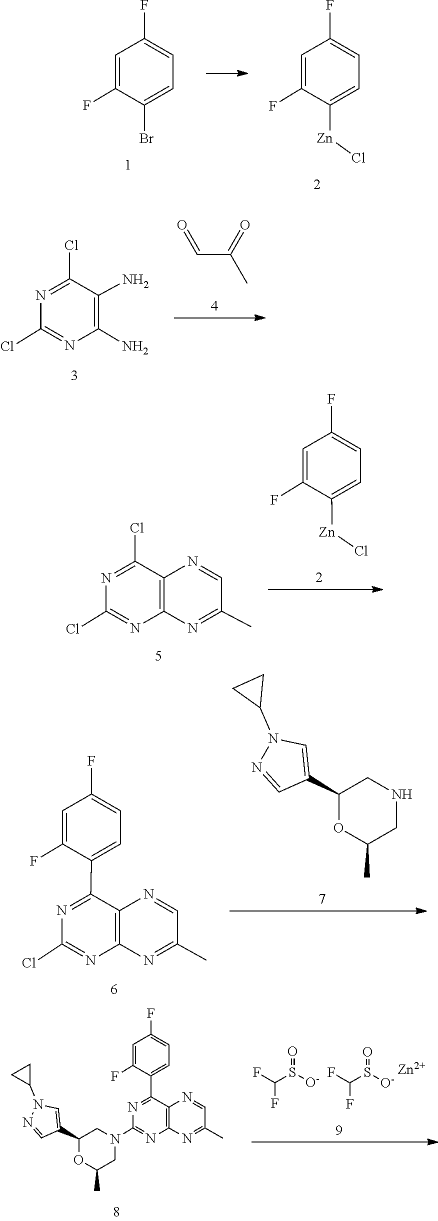

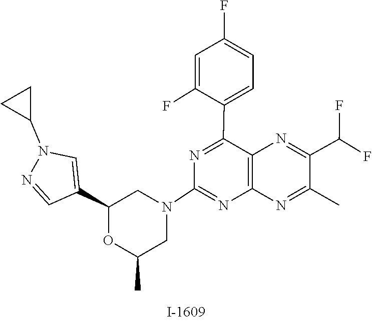 [SMILES: Cc1nc2nc(N3C[C@@H](C)O[C@@H](c4cnn(C5CC5)c4)C3)nc(-c3ccc(F)cc3F)c2nc1C(F)F]

Step 1: A three necked bottle was equipped with 2,4-difluoro-1-iodo-benzene (1.00 eq, 1200 mg, 5.00 mmol), the flash was sealed and purged with N₂ for 3 times, THF (25 mL) was added and the solution was cooled to −40° C. with stirring, i-PrMgCl LiCl (1.3 M in THF) (1.10 eq, 4.2 mL, 5.50 mmol) was added dropwise at −40° C. and stirred for 30 min at this temperature. The reaction mixture was further cooled to −60° C. and zinc (II) chloride (0.5 M in THF) (1.00 eq, 10 mL, 5.00 mmol) was added dropwise, the reaction solution turned into white floc. The reaction mixture was allowed to warm to 15° C. and was stirred for 1 hr. The white floc turned into colorless solution and then used to next step.

Step 2: To a solution of 2,6-dichloropyrimidine-4,5-diamine (1.00 eq, 4000 mg, 22.3 mmol) in DCE (240 mL) was added CaSO₄ (3.00 eq, 9126 mg, 67.0 mmol) followed by 2-oxopropanal (2.50 eq, 4025 mg, 55.9 mmol). The reaction mixture was stirred at 15° C. for 12 hrs. LCMS showed the major peak showed desired MS (M+H)⁺=215.0, purity=99%, UV=220 nm. Retention time=0.382 min. TLC (PE/EA=1/1) showed raw material (Rf=0.3) was consumed completely and the new spot (Rf=0.5) formed. The solution was filtered and then the filtration was concentrated in vacuo to give 2,4-dichloro-7-methyl-pteridine (3400 mg, 15.8 mmol, 70.76% yield) as white solid. MS (M+H)⁺=215.0, purity=99%, uv=220 nm. Retention time=0.382 min.

Step 3: A sealed bottle under N₂ atmosphere was charged with 2,4-dichloro-7-methyl-pteridine (1.00 eq, 800 mg, 3.72 mmol) and PdCl₂(Amphos) (0.0500 eq, 132 mg, 0.186 mmol) and THF (10 mL) and purged with N₂ three times, then cooled to 0° C., chloro-(2,4-difluorophenyl)zinc (1.00 eq, 796 mg, 3.72 mmol) was added dropwise to the reaction solution at 0° C., then warmed to 15° C. and stirred for 16 hr. LCMS showed 39% raw material remained and desired MS (M+H)⁺=293.0, purity=35.4%, uv=220 nm. Retention time=0.846 min. The reaction was poured into water (100 mL) slowly and then extracted with ethyl acetate (20 mL*3) and the organics washed with 20 mL saturated brine solution. The organics were then separated and dried (Na₂SO₄) before concentration to dryness. The crude was then purified by silica gel column (PE/EA=2/1) to give the crude product 700 mg (44% purity). ¹H NMR (400 MHz, CDCl3) δ ppm 2.89-2.93 (m, 3H) 7.02 (ddd, J=9.88, 8.88, 2.38 Hz, 1H) 7.08-7.16 (m, 1H) 7.80 (td, J=8.19, 6.38 Hz, 1H) 8.87 (s, 1H). And then the crude product was purified by prep-HPLC (120 g Flash Column, Welch Ultimate XB_C18 20-40 m; 120 Å, 30% 15 min) to give 2-chloro-4-(2,4-difluorophenyl)-7-methyl-pteridine (170 mg, 0.581 mmol, 15.61% yield) as light red solid, MS (M+H)⁺=293.0, purity=91.088%, uv=220 nm. Retention time=0.854 min.

Step 4: To a solution of 2-chloro-4-(2,4-difluorophenyl)-7-methyl-pteridine (1.00 eq, 150 mg, 0.513 mmol) in DMSO (2 mL) was added (2S,6R)-2-(1-cyclopropylpyrazol-4-yl)-6-methyl-morpholine (1.50 eq, 159 mg, 0.769 mmol) and DIEA (5.00 eq, 331 mg, 2.56 mmol) and then stirred for 20 min at 100° C. LCMS showed raw material was consumed and the major peak showed desired MS (M+H)⁺=464.2, purity=81.74%, uv=220 nm. Retention time=0.936 min. The reaction was poured into water (20 mL) and then extracted with ethyl acetate (10 mL*2) and the organics was washed with 5 mL saturated brine solution. The organics were then separated and dried (Na₂SO₄) before concentration to dryness. The crude was then purified by silica gel column (PE/EA=1/1, Rf=0.5) to give (2S,6R)-2-(1-cyclopropylpyrazol-4-yl)-4-[4-(2,4-difluorophenyl)-7-methyl-pteridin-2-yl]-6-methyl-morpholine (190 mg, 0.410 mmol, 79.99% yield) as red solid. MS (M+H)⁺=464.2, purity=81.74%, uv=220 nm. Retention time=0.936 min. ¹H NMR (400 MHz, CDCl3) δ ppm 0.98-1.04 (m, 2H) 1.10-1.14 (m, 2H) 1.33 (d, J=6.13 Hz, 3H) 2.70-2.77 (m, 3H) 2.87 (dd, J=13.32, 10.69 Hz, 1H) 3.10 (dd, J=13.32, 10.94 Hz, 1H) 3.58 (tt, J=7.16, 3.60 Hz, 1H) 3.76-3.91 (m, 1H) 4.61 (br d, J=10.26 Hz, 1H) 4.99-5.09 (m, 1H) 6.93-7.10 (m, 2H) 7.54 (s, 2H) 7.69 (q, J=7.59 Hz, 1H) 8.41 (s, 1H).

Step 5: To a solution of (2S,6R)-2-(1-cyclopropylpyrazol-4-yl)-4-[4-(2,4-difluorophenyl)-7-methyl-pteridin-2-yl]-6-methyl-morpholine (1.00 eq, 140 mg, 0.302 mmol) and zinc difluoromethanesulfinate (4.00 eq, 355 mg, 1.21 mmol) in DMSO (2.5 mL) at 20° C. was added the solution of tert-butylhydroperoxide (7.00 eq, 190 mg, 2.11 mmol) in DMSO (0.5 mL) with vigorous stirring and bubbled with N₂ for 30 seconds. The reaction solution was stirred at 30° C. for 4 hrs. LCMS showed raw material was consumed and the major peak showed desired MS (M+H)⁺=514.1, purity=53.34%, uv=220 nm. Retention time=1.005 min. The reaction was poured into Na₂SO₃ (aq. 5 mL) and water (10 mL) then was extracted with ethyl acetate (5 mL*2) and the organics was washed with 5 mL saturated brine solution. The organics were then separated and dried (Na₂SO₄) before concentration to dryness. The crude was then purified by silica gel column (PE/EA=1/2) to give 80 mg crude product (93.918% purity) and then purified with prep-TLC (PE/EA=1/2, Rf=0.5) and freeze-drying to give (2S,6R)-2-(1-cyclopropylpyrazol-4-yl)-4-[6-(difluoromethyl)-4-(2,4-difluorophenyl)-7-methyl-pteridin-2-yl]-6-methyl-morpholine (35 mg, 0.0668 mmol, 22.12% yield) as yellow solid. MS (M+H)⁺=514.2, purity=98.733%, uv=220 nm. Retention time=0.997 min. ¹H NMR (400 MHz, CDCl3) δ ppm 1.02 (br d, J=6.60 Hz, 2H) 1.09-1.15 (m, 2H) 1.35 (br d, J=5.87 Hz, 3H) 2.86-2.93 (m, 4H) 3.13 (br t, J=12.04 Hz, 1H) 3.50-3.64 (m, 1H) 3.76-3.90 (m, 1H) 4.53-4.70 (m, 1H) 4.90-5.27 (m, 2H) 6.45-6.79 (m, 1H) 6.92 (s, 2H) 7.49-7.59 (m, 2H) 7.63-7.77 (m, 1H).

### Synthesis of Compound I-1624

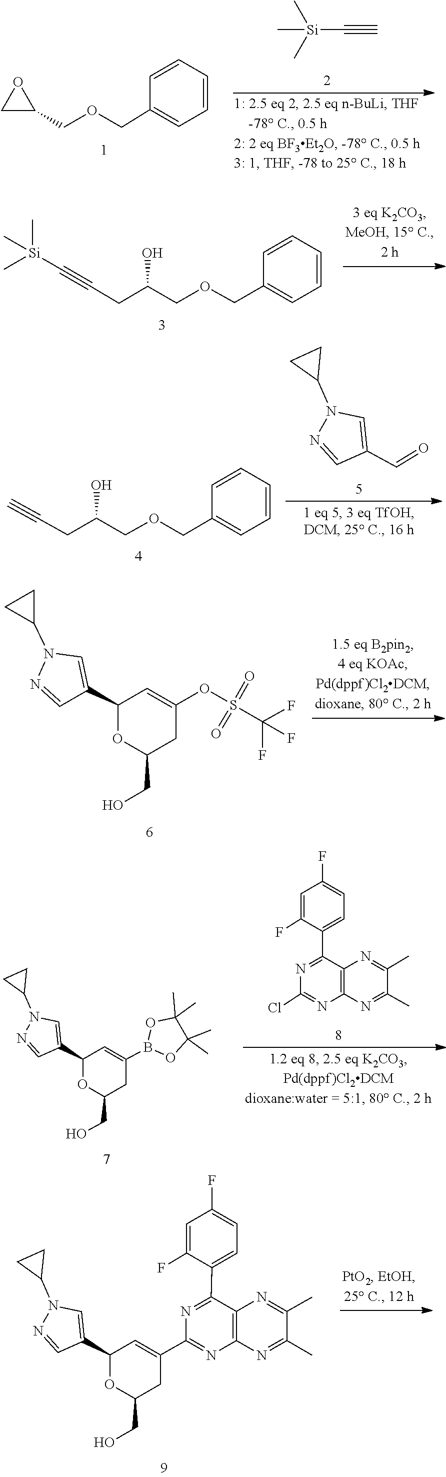

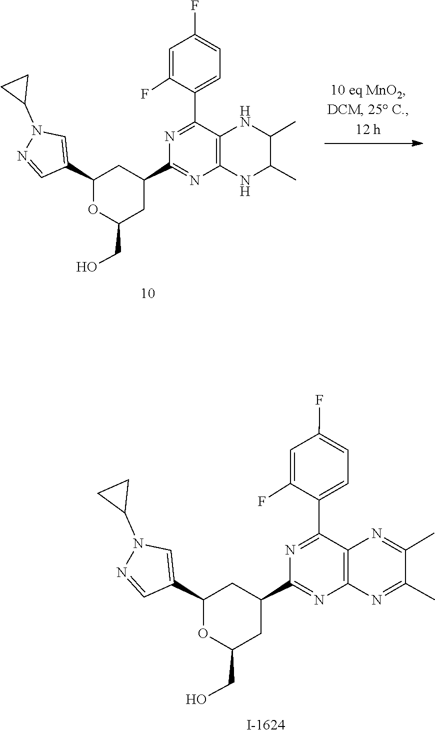 [SMILES: CC1Nc2nc([C@H]3C[C@@H](CO)O[C@@H](c4cnn(C5CC5)c4)C3)nc(-c3ccc(F)cc3F)c2NC1C ; Cc1nc2nc([C@H]3C[C@@H](CO)O[C@@H](c4cnn(C5CC5)c4)C3)nc(-c3ccc(F)cc3F)c2nc1C]

Step 1: To a colorless solution of ethynyltrimethylsilane (2.50 eq, 11 mL, 76.1 mmol) in THF (30 mL) was added n-BuLi (2.5M in Hexanes) (2.50 eq, 30 mL, 76.1 mmol) at −78° C. in N₂, to give a colorless solution, the mixture was stirred at 0° C. for 0.5 hour, the mixture was colorless solution. The mixture was cooled to −78° C. To the mixture was added (2S)-2-(benzyloxymethyl)oxirane (1.00 eq, 5.00 g, 30.5 mmol) in THF (20 mL) and BF₃ Et₂O (2.00 eq, 7.7 mL, 60.9 mmol) at −78° C. in N₂, to give a black solution, the mixture was stirred at −78° C. for 3 hours in N₂. The mixture was colorless solution. LCMS showed the starting material was consumed completely and desired mass was detected (263.3=[M+H]⁺, ESI⁺). The crude reaction mixture was quenched with saturated ammonium chloride (200 mL) at 0° C. The aqueous phase was extracted with ethyl acetate (200 mL*3).The combined organic phase was washed with brine (200 mL*3), dried with anhydrous Na₂SO₄, filtered and concentrated to give a crude product in vacuum. The crude product was purified by column chromatography on silica gel eluted with EtOAc (0 to 20%) in PE. (2S)-1-benzyloxy-5-trimethylsilyl-pent-4-yn-2-ol (7.80 g, 27.9 mmol, 91.66% yield) was obtained as yellow oil, [M+H]⁺=263.1; purity=93.9% (220 nm). Retention time=0.953 min.

Step 2: To a yellow solution of (2S)-1-benzyloxy-5-trimethylsilyl-pent-4-yn-2-ol (1.00 eq, 2.00 g, 7.62 mmol) in Methanol (20 mL) was added K₂CO₃ (3.00 eq, 3160 mg, 22.9 mmol), to give a yellow suspension, the mixture was stirred at 15° C. for 2 hours. The mixture turned into yellow suspension. LCMS showed the starting material was consumed completely and weak desired mass was detected (191.1=[M+H]⁺, ESI⁺). The mixture was concentrated to give a crude product. The crude product was poured into water (50 mL), the aqueous phase was extracted with EA (50 mL*3). The combined organic phase was washed with brine (20 mL*3), dried with anhydrous Na₂SO₄, filtered and concentrated to give a crude product in vacuum. (2S)-1-benzyloxypent-4-yn-2-ol (1270 mg, 6.58 mmol, 86.28% yield) was obtained as colorless oil. [M+H]⁺=191.3; purity=98.5% (220 nm). Retention time=0.765 min. 1H NMR (400 MHz, CDCl₃) δ=7.33-7.18 (m, 5H), 4.49 (s, 2H), 3.94-3.85 (m, 1H), 3.57-3.49 (m, 1H), 3.46-3.36 (m, 1H), 2.47-2.34 (m, 3H), 2.01-1.85 (m, 1H).

Step 3: To a stirred mixture (2S)-1-benzyloxypent-4-yn-2-ol (1.00 eq, 1000 mg, 5.26 mmol) and 1-cyclopropylpyrazole-4-carbaldehyde (1.00 eq, 716 mg, 5.26 mmol) was added in DCM (25 mL) and after stirred at −15° C. for 30 minutes under nitrogen atmosphere. To the mixture was dropwise added TfOH (3.00 eq, 1.4 mL, 15.8 mmol) at −15 to −5° C. under nitrogen atmosphere and stirred the mixture at −10 to 0° C. for 1 h. Then the mixture was stirred at 10 to 25° C. for 16 hours. LCMS (5-95AB/1.5 min): RT=1.025 min, 459.3=[M+H]⁺, ESI⁺ showed 29.3% of desired product (containing Bn protection group) and RT=0.856 min, 369.2=[M+H]⁺, ESI⁺ showed 7% of desired product. To the mixture was added dilute 1 mol HCl cautiously at 0° C. and then adjusted to pH 7 by saturated NaHCO₃ aqueous solution. The mixture was extracted with ethyl acetate (100 mL*3). The combined organic layers were dried over Na₂SO₄, filtered and concentrated under reduced pressure to give a residue. The residue was purified by prep-HPLC (Column, [Phenomenex luna C18 250*50 mm*10 um]; mobile phase: [ACN] and [H₂O] (conditions: [water (0.225% FA)-ACN], B %: 65%-90%; Detector, UV 254 nm. RT: [22 min]) afford [(2S,6R)-6-(1-cyclopropylpyrazol-4-yl)-2-(hydroxymethyl)-3,6-dihydro-2H-pyran-4-yl] trifluoromethanesulfonate (270 mg, 0.711 mmol, 13.53% yield) as green solid. [M+H]⁺=369.0; purity=97% (220 nm). Retention time=0.848 min.

Step 4: To a solution of [(2S,6R)-6-(1-cyclopropylpyrazol-4-yl)-2-(hydroxymethyl)-3,6-dihydro-2H-pyran-4-yl] trifluoromethanesulfonate (1.00 eq, 260 mg, 0.706 mmol), B₂pin₂ (1.50 eq, 269 mg, 1.06 mmol) and potassium acetate (4.00 eq, 277 mg, 2.82 mmol) in 1,4-Dioxane (5 mL), Pd(dppf)Cl₂·CH₂Cl₂ (0.0300 eq, 17 mg, 0.0212 mmol) was added. The reaction mixture was purged with N₂ for 3 times and heated at 80° C. for 4 hours under N₂ atmosphere. LCMS (5-95AB/1.5 min): RT=0.835 min, 347.1=[M+H]⁺, ESI⁺ showed 73.8% of desired product. The reaction mixture was extracted with ethyl acetate (50 mL*3). The combined organic layers were dried over Na₂SO₄, filtered and concentrated under reduced pressure to give a residue. The residue was purified by prep-TLC (PE:EtOAc=0:1, Rf=0.35) to afford [(2S,6R)-6-(1-cyclopropylpyrazol-4-yl)-4-(4,4,5,5-tetramethyl-1,3,2-dioxaborolan-2-yl)-3,6-dihydro-2H-pyran-2-yl]methanol (150 mg, 0.433 mmol, 61.37% yield) as reddish brown solid. [M+H]⁺=347.2; purity=100% (220 nm). Retention time=0.831 min.

Step 5: To a solution of [(2S,6R)-6-(1-cyclopropylpyrazol-4-yl)-4-(4,4,5,5-tetramethyl-1,3,2-dioxaborolan-2-yl)-3,6-dihydro-2H-pyran-2-yl]methanol (1.00 eq, 140 mg, 0.404 mmol) and 2-chloro-4-(2,4-difluorophenyl)-6,7-dimethyl-pteridine (1.20 eq, 149 mg, 0.485 mmol) in 1,4-Dioxane (2.5 mL) and water (0.5000 mL) was added K₂CO₃ (2.50 eq, 140 mg, 1.01 mmol) and Pd(dppf)Cl₂·CH₂Cl₂ (0.1000 eq, 5.2 mg, 0.0404 mmol) at N₂ atmosphere, then the mixture was stirred at 80° C. for 20 minutes. LCMS (5-95AB/1.5 min): RT=0.894 min, 491.2=[M+H]⁺, ESI⁺ showed 64.7% of desired product. The reaction mixture was extracted with ethyl acetate (100 mL*3). The combined organic layers were dried over Na₂SO₄, filtered and concentrated under reduced pressure to give a residue. The crude product was purified by silica gel chromatography using 100% ethyl acetate to afford the [(2S,6R)-6-(1-cyclopropylpyrazol-4-yl)-4-[4-(2,4-difluorophenyl)-6,7-dimethyl-pteridin-2-yl]-3,6-dihydro-2H-pyran-2-yl]methanol (100 mg, 0.165 mmol, 40.84% yield) as a reddish brown solid. [M+H]⁺=491.2; purity=81% (220 nm). Retention time=0.897 min.

Step 6: A solution of [(2S,6R)-6-(1-cyclopropylpyrazol-4-yl)-4-[4-(2,4-difluorophenyl)-6,7-dimethyl-pteridin-2-yl]-3,6-dihydro-2H-pyran-2-yl]methanol (1.00 eq, 90 mg, 0.183 mmol) in ethanol (10 mL) was added PtO₂ (1.00 eq, 42 mg, 0.183 mmol) at H₂ (15 psi) atmosphere and stirred at 25° C. for 16 hours under H₂ (15 psi). LCMS (5-95AB/1.5 min): RT=0.775 min, 497.2=[M+H]⁺, ESI⁺ showed 98% of desired product. The reaction mixture was filtered through a pad of celite. The filter cake was washed with DCM (20 mL). The organic phase was concentrated under reduced pressure to afford [(2S,4R,6R)-6-(1-cyclopropylpyrazol-4-yl)-4-[4-(2,4-difluorophenyl)-6,7-dimethyl-5,6,7,8-tetrahydropteridin-2-yl]tetrahydropyran-2-yl]methanol (100 mg, 0.197 mmol, 107.56% yield) as reddish brown solid. [M+H]⁺=497.2; purity=98% (220 nm). Retention time=0.775 min.

Step 7: To a solution of [(2S,4R,6R)-6-(1-cyclopropylpyrazol-4-yl)-4-[4-(2,4-difluorophenyl)-6,7-dimethyl-5,6,7,8-tetrahydropteridin-2-yl]tetrahydropyran-2-yl]methanol (1.00 eq, 90 mg, 0.181 mmol) in DCM (10 mL), MnO₂ (10.0 eq, 158 mg, 1.81 mmol) was added, the mixture was stirred at 30° C. for 16 hours. LCMS (5-95AB/1.5 min): RT=0.872 min, 493.2=[M+H]⁺, ESI⁺ showed 92% of desired product. The reaction mixture was filtered through a pad of celite. The filter cake was washed with DCM (10 mL). The organic phase was concentrated under reduced pressure to afford a residue. The residue was purified by was purified by prep-HPLC (Column, water Xbridge 150*25 mm * 5 um; mobile phase: [ACN] and [H₂O] (conditions: water (NH₄HCO₃)-ACN, B %: 32%-62%; Detector, UV 254 nm. RT: [8 min]). The purified solution was lyophilized to afford the product [(2S,4R,6R)-6-(1-cyclopropylpyrazol-4-yl)-4-[4-(2,4-difluorophenyl)-6,7-dimethyl-pteridin-2-yl]tetrahydropyran-2-yl]methanol (3.9 mg, 0.00780 mmol, 4.30% yield) as white solid. (5-95AB/1.5 min): RT=0.874 min, 493.2=[M+H]⁺, HPLC RT=0.874 min, 493.2=[M+H]⁺, purity=99.2% (220 nm). Retention time=0.874 min. 1H NMR (400 MHz, CDCl₃) δ=7.80-7.70 (m, 1H), 7.51 (s, 2H), 7.14-6.97 (m, 2H), 4.66 (dd, J=1.7, 11.4 Hz, 1H), 3.97-3.84 (m, 1H), 3.80-3.48 (m, 4H), 2.85 (s, 3H), 2.73 (s, 3H), 2.46 (br d, J=13.3 Hz, 1H), 2.24-2.12 (m, 3H), 2.01-1.89 (m, 1H), 1.15-1.06 (m, 2H), 1.05-0.97 (m, 2H). SFC showed only one peak. The product was prepared from chiral epoxide, so it should be a (2S,4R,6R) product.

### Synthesis of Compounds I-1633

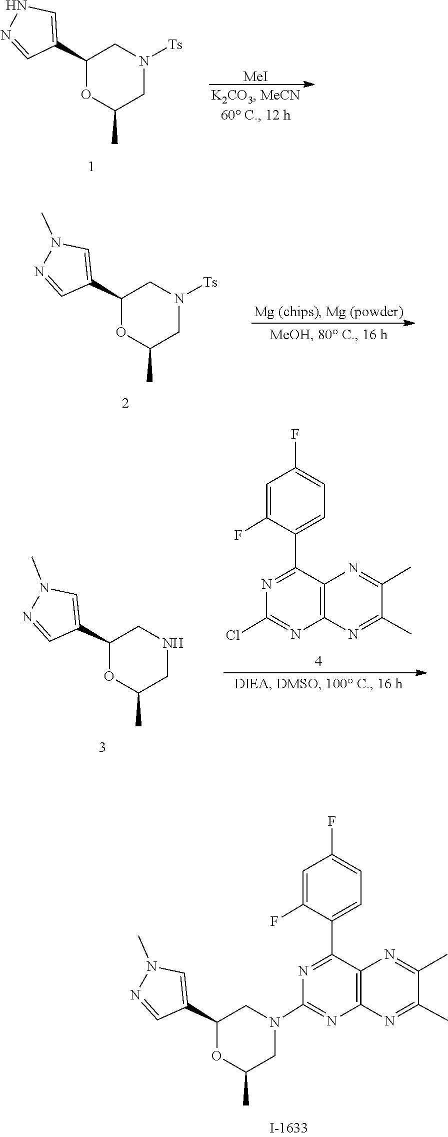 [SMILES: "MeI" ; Cc1ccc(S(=O)(=O)N2C[C@@H](C)O[C@@H](c3cn[nH]c3)C2)cc1 ; Cc1ccc(S(=O)(=O)N2C[C@@H](C)O[C@@H](c3cnn(C)c3)C2)cc1 ; C[C@@H]1CNC[C@H](c2cnn(C)c2)O1 ; Cc1nc2nc(Cl)nc(-c3ccc(F)cc3F)c2nc1C ; Cc1nc2nc(N3C[C@@H](C)O[C@@H](c4cnn(C)c4)C3)nc(-c3ccc(F)cc3F)c2nc1C]

Step 1: To a solution of (2R,6S)-2-methyl-4-(p-tolylsulfonyl)-6-(1H-pyrazol-4-yl) morpholine (1.00 eq, 400 mg, 1.24 mmol) in MeCN (8 mL) was added K₂CO₃ (2.00 eq, 344 mg, 2.49 mmol) and Mel (1.70 eq, 0.13 mL, 2.12 mmol). The mixture was stirred at 60° C. for 12 hours. LCMS showed the starting material was nearly consumed completely and a new peak with desired product mass (71.6%, Rt: 0.562 min; [M+H]⁺=336.1 at 220 nm). The mixture was extracted with EtOAc (300 mL). The combined organic layer was dried over anhydrous sodium sulphate, concentrated under reduced pressure to get the crude residue. The residue was purified by column chromatography on silica gel (eluted with petroleum ether/ethyl acetate=0:1 to 3:1, R_{f}=0.5) to afford (2R,6S)-2-methyl-6-(1-methylpyrazol-4-yl)-4-(p-tolylsulfonyl) morpholine (250 mg, 0.745 mmol, 59.89% yield) as white solid. (M+H)⁺=336.1; purity=71% (220 nm). Retention time=0.562 min. ¹H NMR (400 MHz, CDCl3) δ ppm 1.15 (d, J=6.13 Hz, 3H) 2.00 (t, J=10.82 Hz, 1H) 2.18 (t, J=11.01 Hz, 1H) 2.41 (s, 3H) 3.59 (br d, J=11.26 Hz, 1H) 3.68 (br d, J=11.38 Hz, 1H) 3.81 (s, 3H) 4.60 (dd, J=10.51, 2.38 Hz, 1H) 7.22 (s, 1H) 7.31 (d, J=8.00 Hz, 2H) 7.36 (s, 1H) 7.59 (d, J=8.00 Hz, 2H).

Step 2: To a solution of (2R,6S)-2-methyl-6-(1-methylpyrazol-4-yl)-4-(p-tolylsulfonyl) morpholine (1.00 eq, 250 mg, 0.745 mmol) in methanol (5 mL) was added Mg (chips) (10.0 eq, 179 mg, 7.45 mmol) and Mg (powder) (10.0 eq, 179 mg, 7.45 mmol), then the mixture was stirred at 80° C. for 12 hours under N₂ atmosphere to give white solution. LCMS showed the starting material still remained. Then the reaction mixture was added Mg (chips) (10.0 eq, 179 mg, 7.45 mmol) and stirred at 80° C. for 12 hours under N₂ atmosphere. LCMS showed the starting material was consumed completely and a new peak with desired MS (80%, Rt: 0.14 min, [M+H]⁺=182.1 at 220 nm). The reaction mixture was filtered by celite to afford crude product, the crude product was used directly for the next step. (M+H)⁺=182.1; purity=85% (220 nm). Retention time=0.147 min.

Step 3: To a solution of 2-chloro-4-(2, 4-difluorophenyl)-6, 7-dimethyl-pteridine (1.00 eq, 100 mg, 0.326 mmol) and (2R,6S)-2-methyl-6-(1-methylpyrazol-4-yl) morpholine (2.00 eq, 118 mg, 0.652 mmol) in DMSO (4 mL) was added DIEA (5.00 eq, 0.27 mL, 1.63 mmol). The mixture was stirred at 100° C. for 2 hours. LCMS showed the starting material was consumed completely and a major peak with desired product mass (53%, MS: 452.2 [M+H]⁺, ESI pos). The reaction was filtered and the filtrate was purified by prep-HPLC (flow: 25 mL/min; gradient: from 44-74% water (0.1% FA)-ACN over 7 min; column: Phenomenex Luna C18 150*25 mm*10 um) and lyophilized to afford (2R,6S)-4-[4-(2,4-difluorophenyl)-6,7-dimethyl-pteridin-2-yl]-2-methyl-6-(1-methylpyrazol-4-yl)morpholine (29 mg, 0.0632 mmol, 19.38% yield) as yellow solid. (M+H)⁺=452.2; purity=100% (220 nm). Retention time=0.919 min. HPLC: Retention time=2.255 min, 98.61% purity at 220 nm. ¹H NMR (400 MHz, CDCl3) δ ppm 1.33 (br d, J=5.75 Hz, 3H) 2.60 (s, 3H) 2.72 (s, 3H) 2.81-2.91 (m, 1H) 3.09 (br t, J=11.82 Hz, 1H) 3.85 (br s, 1H) 3.91 (s, 3H) 4.57-4.71 (m, 1H) 4.91-5.19 (m, 2H) 6.92-7.12 (m, 2H) 7.46 (s, 1H) 7.56 (s, 1H) 7.72 (q, J=7.30 Hz, 1H).

### Synthesis of 1-1653

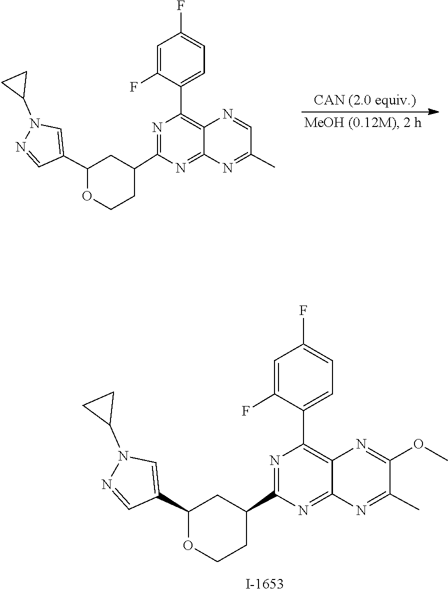 [SMILES: Cc1cnc2c(-c3ccc(F)cc3F)nc(C3CCOC(c4cnn(C5CC5)c4)C3)nc2n1 ; COc1nc2c(-c3ccc(F)cc3F)nc([C@H]3CCO[C@@H](c4cnn(C5CC5)c4)C3)nc2nc1C]

CAN (1.50 eq, 183 mg, 0.33 mmol) was added portion wise to a solution of 2-[2-(1-cyclopropylpyrazol-4-yl)tetrahydropyran-4-yl]-4-(2,4-difluorophenyl)-7-methyl-pteridine- (1.00 eq, 100 mg, 0.22 mmol) in anhydrous methanol (5 mL) at room temperature and the reaction mixture was stirred for 1 h. At this point, CAN (0.5 eq, 63 mg) was added, and the resulting solution was stirred for 1 h. When the reaction was judged complete by LCMS, the reaction mixture washed with water and the aqueous layer was extracted with EtOAc (3×10 mL). The combined organic phases were washed with brine, dried over Na₂SO₄, filtered and concentrated under reduced pressure. The crude residue was purified by reverse phase chromatography using a gradient from 35% to 80% ACN in water before purification with preparative HPLC (conditions: 65% to 0% 10 mM Ammonium bicarbonate pH=10.0 in water) to provide 4-(2,4-difluorophenyl)-6-methoxy-7-methyl-2-[rac-(2R,4S)-2-(1-cyclopropylpyrazol-4-yl)tetrahydropyran-4-yl]pteridine as a yellow solid (26.6 mg, 0.054 mmol, 24% yield). ESI-MS (m/z+): 479.3 [M+1]⁺, LC-RT: 1.60 min. ¹H NMR (DMSO-d₆, 400 MHz): δ_{H}7.83 (1H, td, J=8.4, 6.6 Hz), 7.69 (1H, s), 7.47-7.41 (1H, m), 7.35 (1H, d, J=0.8 Hz), 7.27 (1H, td, J=8.5, 2.6 Hz), 4.47 (1H, d, J=11.1 Hz), 4.06 (1H, dd, J=11.3, 4.2 Hz), 3.90 (2H, s), 3.69-3.58 (2H, m), 3.40 (1H, t, J=11.8 Hz), 2.62 (2H, s), 2.26 (1H, d, J=13.4 Hz), 1.99 (1H, s), 1.92-1.83 (2H, m), 0.90-0.84 (2H, m), 0.98-0.93 (2H, m). ¹⁹F NMR (DMSO-d₆, 376 MHz): δ_{F}−107.2, -107.0.

### Synthesis of I-1658

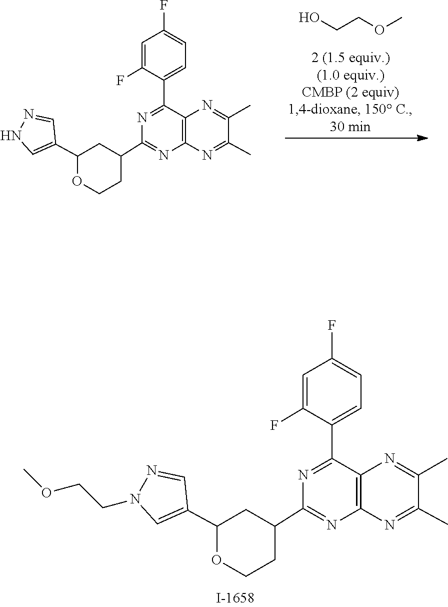

A flame-dried microwave vial under N₂ (g) was charged with 2-(2-(1H-pyrazol-4-yl)tetrahydro-2H-pyran-4-yl)-4-(2,4-difluorophenyl)-6,7-dimethylpteridine (1.0 eq, 60 mg, 0.14 mmol), 2-(tributyl-,5-phosphanylidene)acetonitrile (CPMB, 2.0 eq, 0.075 mL, 0.284 mmol) and 2-methoxyethanol (1.5 eq, 0.017 mL, 0.21 mmol). 1,4-dioxane (1.4 mL) was added and the vial was sealed with an aluminum cap with septa. The reaction mixture was irradiated under constant microwave for 30 min with the reaction temperature controlled at 100° C. The crude reaction was evaporated to dryness under reduced pressure and the residual material was purified by silica gel flash chromatography (Sfar Biotage® column 24 g, using a gradient from 40% EtOAc in DCM to 100% EtOAc) and reverse phase chromatography (Biotage® C18 duo column 12 g, using a gradient from 35% CH₃CN in water to 80% CH₃CN in water) to obtain a solid which was further purified by prep HPLC (Gemini® 5 um NX-C18 110 Å, 100×30 mm column) using MeOH and 10 mM aqueous ammonium formate (50-100%) to afford 4-(2,4-difluorophenyl)-2-(2-(1-(2-methoxyethyl)-1H-pyrazol-4-yl)tetrahydro-2H-pyran-4-yl)-6,7-dimethylpteridine (29 mg, 0.06 mmol, 42% yield) as a single diastereoisomer (cis). ESI-MS (m/z+): 481.4 [M+1]⁺, LC-RT: 1.45 min. ¹H NMR (DMSO-d₆, 400 MHz): ^{6H} 7.77-7.83 (1H, m), 7.67 (1H, s), 7.47 (1H, td, J=9.9, 2.5 Hz), 7.40 (1H, s), 7.31 (1H, td, J=8.5, 2.5 Hz), 4.53 (1H, dd, J=11.3, 2.0 Hz), 4.19 (2H, t, J=5.4 Hz), 4.10 (1H, dd, J=11.3, 4.2 Hz), 3.71 (1H, t, J=11.6 Hz), 3.64 (2H, t, J=5.4 Hz), 3.43-3.51 (1H, m), 3.20 (3H, s), 2.77 (3H, s), 2.65 (3H, s), 2.31 (1H, d, J=13.0 Hz), 2.06 (1H, d, J=13.1 Hz), 1.88-1.97 (2H, m). ¹⁹F NMR (DMSO-d₆, 376 MHz): S_{F}-107.1, -107.7.

### Synthesis of Compound 1-1668

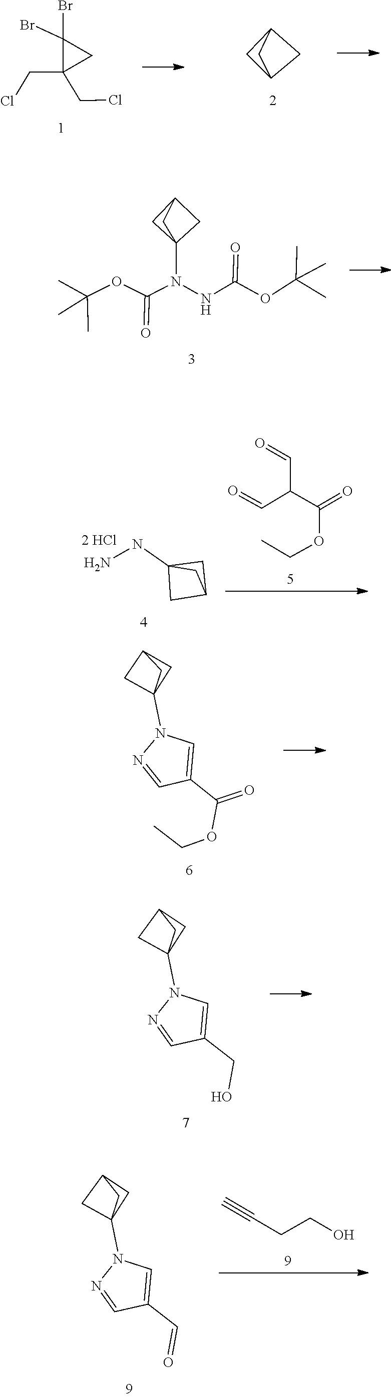

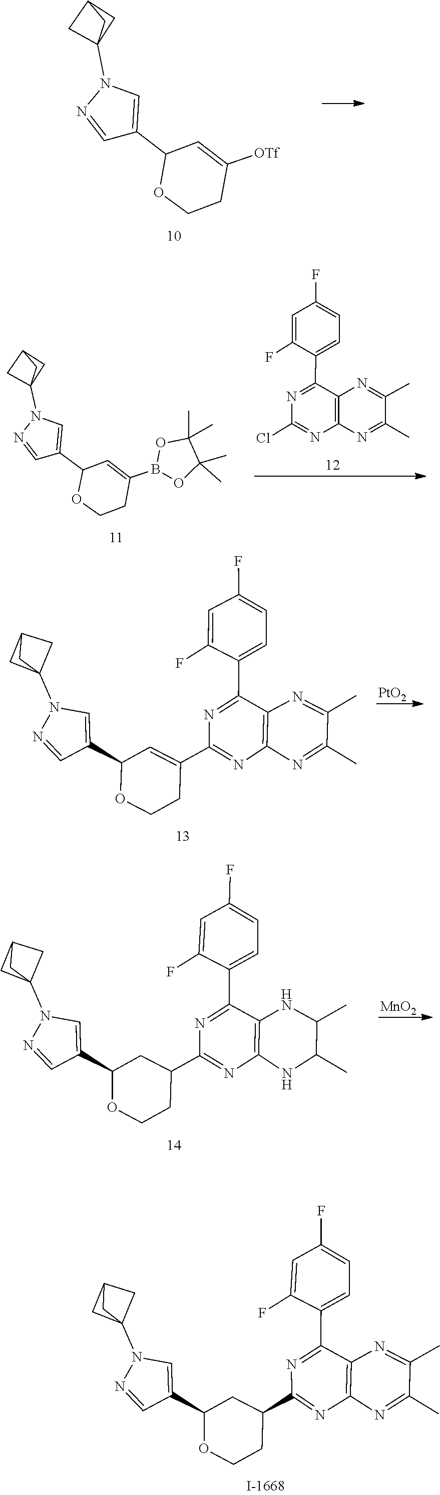

Step 1: A mixture of 1,1-dibromo-2,2-bis(chloromethyl)cyclopropane (1.00 eq, 100.00 g, 337 mmol) in Pentane (200 mL) was degassed with N₂ for 3 times and cooled to −50° C. To the mixture was added MeLi (2.37 eq, 500 mL, 800 mmol) slowly below −50° C. Then the mixture was warmed to 0° C. and stirred for 3 h. The mixture was added to ice water and extracted with DCM (150 mL*3). The organic phase was distilled under vacuum at 20° C. to give a solution tricyclo[1.1.1.01,3]pentane (11.00 g, 166 mmol, 49.40% yield) in DCM/pentane/diethyl ether (1100 mL). The solution was used for next step directly without further purification.

Step 2: A mixture of tris(2,2,6,6-tetramethyl-3,5-heptanedionato) manganese(III) (0.0500 eq, 2287 mg, 3.78 mmol) in 1-propanol (500 mL) was cooled to 0° C. Then a solution of PHSiH₃ (1.00 eq, 8186 mg, 75.6 mmol) and tricyclo[1.1.1.01,3]pentane (1.00 eq, 5000 mg, 75.6 mmol) in DCM (500 mL) was added at 0° C. Next, tert-butyl-N-tert-butoxycarbonyliminocarbamate (1.50 eq, 26126 mg, 113 mmol) in Et₂O/Pentane/DCM (100 mL) was added at 0° C. The mixture was stirred at 0° C. for 3 h. The mixture was added 30 mL water and concentrated under vacuum to give a crude. The crude was purified by flash column (PE to 5% EtOAc in PE; Cerium (IV)-Phosphomolybdic acid stain, PE:EtOAc=5: 1, the desired product Rf-0.4) to give tert-butyl N-(1-bicyclo[1.1.1]pentanyl)-N-(tert-butoxycarbonylamino)carbamate (7.90 g, 26.5 mmol, 35.00% yield) as off-white solid. 1H NMR (400 MHz, CDCl3) δ=2.11-1.93 (m, 6H), 1.56-1.39 (m, 20H).

Step 3: A mixture of tert-butyl N-(1-bicyclo[1.1.1]pentanyl)-N-(tert-butoxycarbonylamino)carbamate (1.00 eq, 4000 mg, 13.4 mmol) in HCl/EtOAc (11.9 eq, 40 mL, 160 mmol) was stirred at 20° C. for 12 h. The mixture was filtered and the filter cake was concentrated under vacuum to give 1-bicyclo[1.1.1]pentanylhydrazine; dihydrochloride (2000 mg, 11.7 mmol, 87.21% yield) as white solid. 1H NMR (400 MHz, DMSO-d6) δ=2.45 (s, 1H), 1.82 (s, 6H).

Step 4: A solution of 1-bicyclo[1.1.1]pentanylhydrazine; dihydrochloride (1.00 eq, 500 mg, 2.92 mmol) in ethanol (5 mL), the mixture was cooled to 0° C., ethyl 2-formyl-3-oxo-propanoate (1.00 eq, 421 mg, 2.92 mmol) in ethanol (5 mL) was added to the reaction mixture. Then the mixture was stirred at 25° C. for 2 h. LCMS monitored the reaction, the starting material was consumed completely and 75% of desired product was detected (75% purity, Rt=0.822 min, [M+H]⁺=207.2 at 220 nm). The mixture was concentrated under reduced pressure to give the residue. The residue was purified by Prep-HPLC (0.5% FA condition) and extracted with EA (100 mL*3), the combine organic layers was dried over anhydrous Na₂SO₄, filtered and concentrated under reduced pressure to give the ethyl 1-(1-bicyclo[1.1.1]pentanyl)pyrazole-4-carboxylate (350 mg, 1.70 mmol, 58.06% yield) as a yellow oil. LCMS [M+H]⁺=207.2, purity=100% (220 nm). Retention time=0.822 min. 1H NMR (400 MHz, CDCl3) δ=7.92 (d, J=10.3 Hz, 2H), 4.30 (q, J=7.1 Hz, 2H), 2.66 (s, 1H), 2.33 (s, 6H), 1.35 (t, J=7.1 Hz, 3H).

Step 5: The mixture was stirred at 15° C. for 4 h. LCMS showed the starting material was consumed completely and 98% of desired product was detected (98%, RT=0.431 min, [M+H]⁺=165.3 at 220 nm). The mixture was quenched by 0.2 mL H₂O, then 10 g Na₂SO₄ and 5 mL EA was added to the reaction, the mixture was stirred at 15° C. for 30 min. After, the mixture was filtered and the filter cake was washed with EA (5 mL*3), the combine organic layers was concentrated under reduced pressure to give crude [1-(1-bicyclo[1.1.1]pentanyl)pyrazol-4-yl]methanol (300 mg, 1.83 mmol, 107.66% yield) as a colorless oil and the residue was used directly. 1H NMR (400 MHz, CDCl3) δ=7.54 (s, 1H), 7.45 (s, 1H), 4.60 (s, 2H), 2.62 (s, 1H), 2.30 (s, 7H).

Step 6: To a solution of [1-(1-bicyclo[1.1.1]pentanyl)pyrazol-4-yl]methanol (1.00 eq, 280 mg, 1.71 mmol) in dry DCE (25 mL) was added MnO₂ (20.0 eq, 2965 mg, 34.1 mmol). The mixture was stirred at 55° C. for 12 h. LCMS showed the starting material consumed completely and one major peak with desired product was detected (75%, Rt=0.583 min, [M+H]⁺=163.1 at 220 nm). The mixture was filtered and the filter cake was washed with EA (20 mL*3), the combine organic layers was concentrated under reduced pressure to give the 1-(1-bicyclo[1.1.1]pentanyl)pyrazole-4-carbaldehyde (195 mg, 1.20 mmol, 70.510% yield) as yellow oil and the residue was used to the next step directly.

Step 7: To a stirred mixture 1-(1-bicyclo[1.1.1]pentanyl)pyrazole-4-carbaldehyde (1.00 eq, 170 mg, 1.05 mmol) and 3-BUTYN-1-OL (1.00 eq, 0.080 mL, 1.05 mmol) was added in DCE (5 mL) and after stirred at −10° C. for 30 mins under nitrogen atmosphere. To the mixture was dropwise added TfOH (3.00 eq, 0.28 mL, 3.14 mmol) at −10° C. under nitrogen atmosphere and stirred the mixture at −10° C. for 1 h. Then the mixture stirred at 15° C. for 16 h. LCMS showed the starting material consumed completely and one major peak with desired product was detected (86%, RT=0.941 min, [M+H]⁺=365.2 at 220 nm). The mixture was quenched by 50 mL H₂O, extracted with EA (100 mL*3), the combine organic layers was dried over anhydrous Na₂SO₄, filtered and concentrated under reduced pressure to give the [6-[1-(1-bicyclo[1.1.1]pentanyl)pyrazol-4-yl]-3,6-dihydro-2H-pyran-4-yl] trifluoromethanesulfonate (320 mg, 0.878 mmol, 83.80% yield) as red oil and the residue was used to the next step directly.

Step 8: A solution of [6-[1-(1-bicyclo[1.1.1]pentanyl)pyrazol-4-yl]-3,6-dihydro-2H-pyran-4-yl] trifluoromethanesulfonate (1.00 eq, 270 mg, 0.741 mmol), B₂pin₂ (1.50 eq, 282 mg, 1.11 mmol) and AcOK (4.00 eq, 290 mg, 2.96 mmol) in 1,4-Dioxane (3 mL) was added Pd(dppf)Cl₂ (0.100 eq, 61 mg, 0.0741 mmol) under N₂ atmosphere. The reaction mixture was heated at 90° C. for 4 h under N₂. LCMS showed the starting material was consumed completely and 22% desired product was detected (22%, Rt=0.917 min, [M+H]⁺=343.2 at 220 nm). The reaction mixture was quenched by 50 mL H₂O, extracted with EA (50 mL*3), the combine organic layers was dried over anhydrous Na₂SO₄, filtered and concentrated under reduced pressure to give. The residue was purified by Prep-HPLC (Phenomenex luna C18 150*25 mm*10 um, water (FA)-ACN) and then extracted with EA (20 mL*3), the combine organic layers was dried over anhydrous Na₂SO₄, filtered and concentrated under reduced pressure to give the 1-(1-bicyclo[1.1.1]pentanyl)-4-[4-(4,4,5,5-tetramethyl-1,3,2-dioxaborolan-2-yl)-3,6-dihydro-2H-pyran-6-yl]pyrazole (60 mg, 0.175 mmol, 23.66% yield) as a light-yellow oil. [M+H]⁺=343.1; purity=84% (220 nm). Retention time=0.915 min. 1H NMR (400 MHz, CDCl3) δ=7.51 (s, 1H), 7.39 (s, 1H), 6.57 (d, J=1.6 Hz, 1H), 5.18 (br d, J=2.2 Hz, 1H), 3.95-3.86 (m, 1H), 3.72 (ddd, J=4.4, 7.4, 11.5 Hz, 1H), 2.59 (s, 1H), 2.27 (s, 8H), 1.28 (s, 13H).

Step 9: To a solution of 2-chloro-4-(2,4-difluorophenyl)-6,7-dimethyl-pteridine (1.00 eq, 42 mg, 0.137 mmol), 1-(1-bicyclo[1.1.1]pentanyl)-4-[4-(4,4,5,5-tetramethyl-1,3,2-dioxaborolan-2-yl)-3,6-dihydro-2H-pyran-6-yl]pyrazole (1.15 eq, 54 mg, 0.157 mmol) and K₂CO₃ (3.00 eq, 35 mg, 0.411 mmol) in 1,4-Dioxane (2 mL) and Water (0.2 mL) was added Pd(dppf)Cl₂ DCM (0.120 eq, 12 mg, 0.0164 mmol). The reaction mixture was stirred at 80° C. for 12 hours under N₂ atmosphere. LCMS showed the starting material was consumed completely and 60% of desired product was detected (60%, Rt=0.998 min, [M+H]⁺=487.3 at 220 nm). The reaction was quenched by 5 mL H₂O, extracted with EA (20 mL*3), the combine organic layers was dried over anhydrous Na₂SO₄, filtered, and concentrated under reduced pressure to give the residue. The residue was purified by silica gel chromatography (PE:EA=1:0 to 1:1, PE:EA=1:1, the desired product Rf=0.3) to give the 2-[6-[1-(1-bicyclo[1.1.1]pentanyl)pyrazol-4-yl]-3,6-dihydro-2H-pyran-4-yl]-4-(2,4-difluorophenyl)-6,7-dimethyl-pteridine (40 mg, 0.0822 mmol, 60.04% yield) as colorless oil, [M+H]⁺=487.3; purity=93% (220 nm). Retention time=0.998 min.

Step 10: To a solution of 2-[(6R)-6-[1-(1-bicyclo[1.1.1]pentanyl)pyrazol-4-yl]-3,6-dihydro-2H-pyran-4-yl]-4-(2,4-difluorophenyl)-6,7-dimethyl-pteridine (1.00 eq, 40 mg, 0.0822 mmol) in Ethanol (2 mL) was added PtO₂ (1.15 eq, 10 mg, 0.0943 mmol) under N₂ atmosphere. The mixture was purged with H₂ (15 psi) 3 times, then the mixture was stirred at 30° C. for 12 h under H₂ (15 psi) atmosphere. LCMS showed the starting material was consumed completely and one major peak with the desired product was detected (91%, Rt=0.893 min [M+H]⁺=493.4 at 220 nm). The reaction mixture was filtered and concentrated under reduced pressure to give the 2-[(2R)-2-[1-(1-bicyclo [1.1.1]pentanyl)pyrazol-4-yl]tetrahydropyran-4-yl]-4-(2,4-difluorophenyl)-6,7-dimethyl-5,6,7,8-tetrahydropteridine (30 mg, 0.0609 mmol, 74.08% yield) as yellow solid and the residue was used to the next step directly.

Step 11: To a solution of 2-[(2R)-2-[1-(1-bicyclo[1.1.1]pentanyl)pyrazol-4-yl]tetrahydropyran-4-yl]-4-(2,4-difluorophenyl)-6,7-dimethyl-5,6,7,8-tetrahydropteridine (1.00 eq, 30 mg, 0.0609 mmol) in dry DCE (2 mL) was added MnO₂ (20.0 eq, 106 mg, 1.22 mmol). The mixture was stirred at 30° C. for 12 h. LCMS showed the starting material was consumed completely and one major peak with desired product was detected (83%, Rt=0.969 min, [M+H]⁺=489.4 at 220 nm). The mixture was filtered and the filter cake was washed with MeOH (10 mL*3), the combine organic layers was concentrated under reduced pressure to give the residue. The residue was purified by Prep-HPLC (Phenomenex C18 75*30 mm*3 um, water (FA)-ACN) and lyophilized to give the 2-[(2R,4S)-2-[1-(1-bicyclo [1.1.1]pentanyl)pyrazol-4-yl]tetrahydropyran-4-yl]-4-(2,4-difluorophenyl)-6,7-dimethyl-pteridine (5.3 mg, 0.0107 mmol, 17.50% yield) as white solid [M+H]⁺=489.3; purity=98.4% (220 nm). Retention time=0.983 min. 1H NMR (400 MHz, CDCl3) δ=7.81-7.72 (m, 1H), 7.56 (s, 1H), 7.51 (s, 1H), 7.10 (qd, J=2.9, 8.8 Hz, 1H), 7.01 (dt, J=2.3, 9.4 Hz, 1H), 4.58 (dd, J=1.7, 11.4 Hz, 1H), 4.28 (td, J=3.0, 11.2 Hz, 1H), 3.91-3.77 (m, 1H), 3.63-3.44 (m, 1H), 2.85 (s, 3H), 2.74 (s, 3H), 2.60 (s, 1H), 2.46 (br d, J=13.0 Hz, 1H), 2.28 (s, 6H), 2.26-2.15 (m, 3H). SFC showed 2 peaks (ratio was 1:1) cis-mixture.

### Synthesis of Compounds I-1673 and I-1674

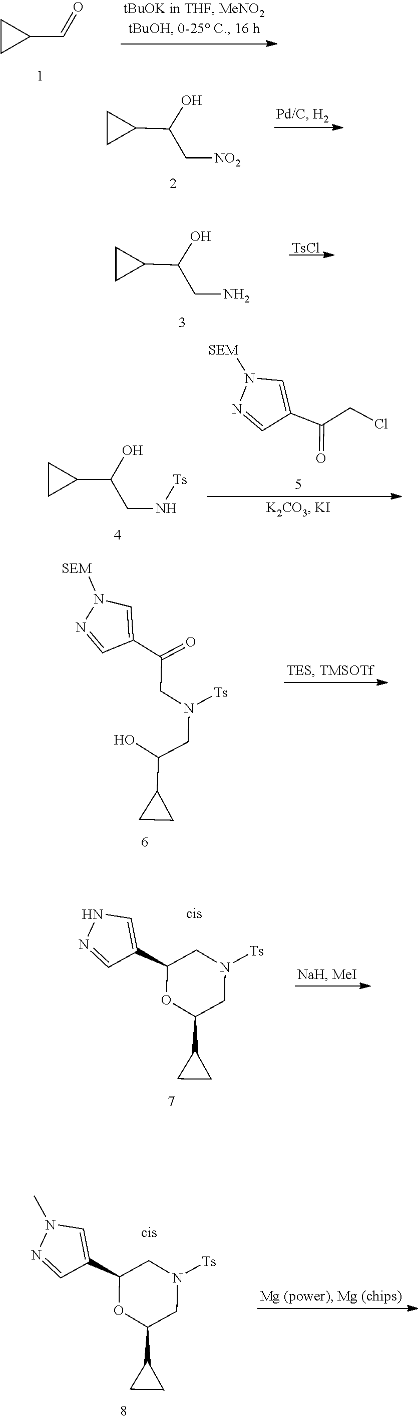

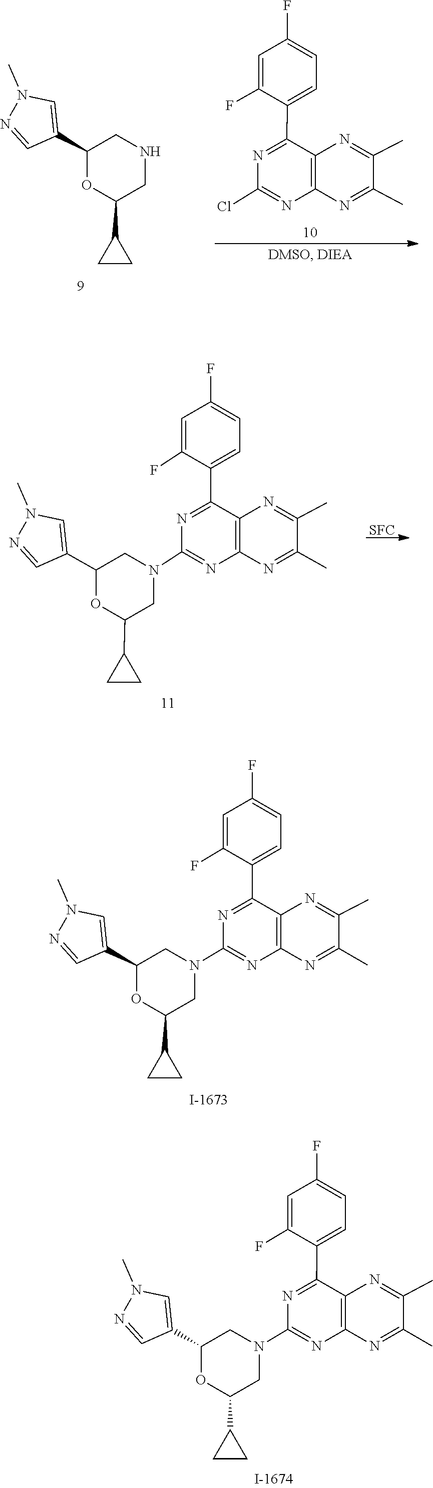

Step 1: A mixture of THF (50 mL) and t-BuOH (50 mL) was cooled to 0° C., cyclopropane-carbaldehyde (1.00 eq, 5.4 mL, 71.3 mmol) and nitromethane (1.50 eq, 5.8 mL, 107 mmol) was added to the reaction mixture. After stirring 5 minutes, t-BuOK (0.200 eq, 14 mL, 14.3 mmol) (in THF) was added slowly. During the addition, a white solid formed. The mixture was allowed to warm to 20° C. and then stirred at 20° C. for 12 h. TLC (PE:EA=10:1/V: V, 2, 4-dinitrobenzene, the starting material Rf=0.5) monitored the reaction, the starting material was consumed completely and one new peak (PE:EA=10:1/V: V, the desired product Rf=0.7) was detected. The mixture was quenched by 150 mL H₂O, extracted with EA (150 mL*3), the combine organic layers was dried over anhydrous Na₂SO₄, filtered and concentrated under reduced pressure to give the 1-cyclopropyl-2-nitro-ethanol (7.20 g, 54.9 mmol, 76.97% yield) as colorless oil, 1H NMR (400 MHz, CDCl3) δ=4.57-4.52 (m, 2H), 3.68 (dt, J=4.3, 7.9 Hz, 1H), 2.40 (br s, 1H), 0.96 (tq, J=4.9, 8.2 Hz, 1H), 0.73-0.57 (m, 2H), 0.52-0.43 (m, 1H), 0.41-0.30 (m, 1H).

Step 2: To the mixture of 1-cyclopropyl-2-nitro-ethanol (1.00 eq, 7.20 g, 54.9 mmol) in Methanol (72 mL) was added Pd/C (0.0124 eq, 0.72 g, 0.679 mmol) at 25° C. The mixture was purged with H₂ several times then the reaction mixture was stirred at 60° C. for 12 h under H₂ (40 Psi) atmosphere. The mixture was filtered and the filter cake washed with MeOH (50 mL*3), the combined organic layers was concentrated under reduced pressure to give the 2-amino-1-cyclopropyl-ethanol (4.40 g, 43.5 mmol, 79.22% yield) as colorless oil. 1H NMR (400 MHz, CDCl3) δ=2.94 (br dd, J=3.2, 12.3 Hz, 1H), 2.89-2.81 (m, 1H), 2.72 (br dd, J=7.9, 12.3 Hz, 1H), 0.84 (br dd, J=4.2, 7.7 Hz, 1H), 0.61-0.45 (m, 2H), 0.35 (br d, J=8.6 Hz, 1H), 0.27-0.16 (m, 1H).

Step 3: To the mixture of 2-amino-1-cyclopropyl-ethanol (1.00 eq, 1.00 g, 9.89 mmol) in DCM (10 mL) was added TEA (1.50 eq, 1498 mg, 14.8 mmol), TsCl (1.10 eq, 2066 mg, 10.9 mmol) at 0° C., then the reaction mixture was stirred at 20° C. for 12 h. LCMS showed the starting material was consumed completely and one main peak (69%, Rt=0.798 min; [M+H]⁺=238.1 at 220 nm) was detected. The reaction mixture was quenched by addition water 10 mL at 20° C., and then diluted with EtOAc 20 mL, extracted with EtOAc 60 mL (20 mL*3). The combined organic layers were washed with aq. sat. NaCl 10 mL (10 mL), dried over [anhydrous Na₂SO₄], filtered and concentrated under reduced pressure to give a residue. The residue was purified by flash silica gel chromatography (ISCO; 40 g SepaFlash Silica Flash Column, Eluent of 0-50% Ethyl acetate/Petroleum ether gradient @ 40 mL/min, PE:EtOAc=3:1, the desired product Rf=0.6) to give N-(2-cyclopropyl-2-hydroxy-ethyl)-4-methyl-benzenesulfonamide (1.40 g, 5.48 mmol, 55.46% yield) as colorless oil, [M+H]⁺=238.1; purity=98% (220 nm). Retention time=0.810 min. 1H NMR (400 MHz, CDCl3) δ=7.76 (d, J=8.3 Hz, 2H), 7.33 (d, J=8.4 Hz, 2H), 4.99-4.84 (m, 1H), 3.28-3.15 (m, 1H), 3.00-2.89 (m, 2H), 2.44 (s, 3H), 0.85 (dq, J=4.3, 8.1 Hz, 1H), 0.56-0.48 (m, 2H), 0.35-0.11 (m, 2H).

Step 4: To a solution of 2-chloro-1-[1-(2-trimethylsilylethoxymethyl)pyrazol-4-yl]ethanone (1.00 eq, 1507 mg, 5.48 mmol) and N-(2-cyclopropyl-2-hydroxy-ethyl)-4-methyl-benzenesulfonamide (1.00 eq, 1400 mg, 5.48 mmol) in acetone (60 mL) was added K₂CO₃ (3.00 eq, 2273 mg, 16.4 mmol) and KI (1.00 eq, 910 mg, 5.48 mmol). The mixture stirred at 25° C. for 2 h. LCMS showed the starting material was remained. The mixture was stirred at 30° C. for another 12 h. LCMS showed 39% of desired product was detected (39%, Rt=1.018 min, [M+H-H₂O]⁺=476.3 at 220 nm). The reaction mixture was partitioned between EtOAc (50*3 mL) and water (50*3 mL). The organic layer was washed with brine, dried by Na₂SO₄. The solution was concentrated to give the residue. The crude product was purified by column chromatography on silica gel (eluted with petroleum ether/ethyl acetate=100: 1 to 1: 1, PE:EA=1:1, the desired product Rf=0.3) to give a crude product. The crude product was purified again by Prep-HPLC (0.5% FA condition), then extracted with EA (150 mL*3), the combine organic layers was dried over anhydrous Na₂SO₄, filtered and concentrated under reduced pressure to give the N-(2-cyclopropyl-2-hydroxy-ethyl)-4-methyl-N-[2-oxo-2-[1-(2-trimethylsilylethoxymethyl)pyrazol-4-yl]ethyl]benzenesulfonamide (880 mg, 1.07 mmol, 19.51% yield) as colorless oil, checked by LCMS: [M+H]⁺=476.3; purity=59% (220 nm). Retention time=0.810 min. 1H NMR (400 MHz, CDCl3) δ=8.16 (s, 1H), 7.98 (s, 1H), 7.76 (d, J=8.3 Hz, 1H), 7.69-7.55 (m, 2H), 7.37-7.30 (m, 2H), 5.46 (s, 1H), 5.40-5.36 (m, 1H), 4.71-4.45 (m, 1H), 3.83-3.71 (m, 1H), 3.67-3.52 (m, 3H), 3.48-3.24 (m, 2H), 3.13-2.99 (m, 1H), 2.47-2.42 (m, 3H), 1.00-0.87 (m, 2H), 0.82-0.71 (m, 1H), 0.65-0.40 (m, 3H), 0.39-0.30 (m, 1H), 0.22-0.13 (m, 1H), 0.03-0.04 (m, 10H).

Step 5: To a solution of N-(2-cyclopropyl-2-hydroxy-ethyl)-4-methyl-N-[2-oxo-2-[1-(2-trimethylsilylethoxymethyl)pyrazol-4-yl]ethyl]benzenesulfonamide (1.00 eq, 800 mg, 1.62 mmol) and TES (10.0 eq, 5.1 mL, 16.2 mmol) in DCM (20 mL) was added TMSOTf (10.0 eq, 2.9 mL, 16.2 mmol) at 0° C., the mixture was stirred at 30° C. for 12 h. LCMS showed the starting material was consumed completely and 98% of desired product was detected (98%, Rt=0.898 min, [M+H]⁺=348.2 at 220 nm). The residue was partitioned between DCM (100*2 mL) and water (30 mL). The separated organic layer was dried over Na₂SO₄ and evaporated to dryness. The crude product was purified by column chromatography on silica gel eluted with petroleum ether/ethyl acetate=1: 0 to 0: 1 (petroleum ether/ethyl acetate=1:1, the desired product Rf=0.2) to give 2-cyclopropyl-4-(p-tolylsulfonyl)-6-(1H-pyrazol-4-yl)morpholine (660 mg, 1.90 mmol, 117.23% yield) as white solid, checked by 1H NMR: (400 MHz, CDCl3) δ=8.22-7.71 (m, 2H), 7.65 (br d, J=7.6 Hz, 2H), 7.37 (br d, J=7.4 Hz, 2H), 4.78-4.56 (m, 1H), 3.84-3.65 (m, 2H), 3.04 (br d, J=7.5 Hz, 1H), 2.46 (s, 3H), 2.31-2.15 (m, 2H), 0.81 (br d, J=3.0 Hz, 1H), 0.57 (br d, J=6.1 Hz, 2H), 0.47-0.26 (m, 2H)).

Step 6: To a solution of (2R,6S)-2-cyclopropyl-4-(p-tolylsulfonyl)-6-(1H-pyrazol-4-yl)morpholine (1.00 eq, 400 mg, 1.15 mmol) in THF (8 mL) was added NaH (1.10 eq, 0.00051 mL, 1.27 mmol) at 0° C., then the mixture was stirred at 25° C. for 0.5 h. Mel (1.10 eq, 180 mg, 1.27 mmol) in THF (1 mL) was added to the reaction mixture, then the mixture was stirred at 25° C. for 12 h. LCMS showed the starting material was consumed completely and one major peak with desired product was detected (33%, Rt=1.006 min, [M+H]⁺=362.3 at 220 nm). The mixture was quenched by 20 mL saturation NH₄Cl, extracted with EA (100 mL*3), the combine organic layers was dried over anhydrous Na₂SO₄, filtered and concentrated under reduced pressure to give the residue. The residue was purified by silica gel chromatography (PE:EA=1:0 to 0:1, PE:EA=0:1, the desired product Rf=0.3) to give the (2R,6S)-2-cyclopropyl-6-(1-methylpyrazol-4-yl)-4-(p-tolylsulfonyl)morpholine (290 mg, 0.802 mmol, 69.69% yield) as colorless oil. LCMS: [M+H]⁺=476.3; purity=98% (220 nm). Retention time=0.903 min. 1H NMR (400 MHz, CDCl3) δ=7.65 (d, J=8.3 Hz, 2H), 7.42-7.31 (m, 4H), 4.57 (dd, J=2.5, 10.5 Hz, 1H), 3.86 (s, 3H), 3.78-3.63 (m, 2H), 3.00 (ddd, J=2.5, 8.2, 10.4 Hz, 1H), 2.46 (s, 3H), 2.28-2.18 (m, 2H), 0.86-0.73 (m, 1H), 0.63-0.49 (m, 2H), 0.46-0.27 (m, 2H).

Step 7: To a solution of (2R,6S)-2-cyclopropyl-6-(1-methylpyrazol-4-yl)-4-(p-tolylsulfonyl)morpholine (1.00 eq, 290 mg, 0.802 mmol) in methanol (16 mL) was added Mg (powder) (15.6 eq, 300 mg, 12.5 mmol) and Mg(chips) (15.6 eq, 300 mg, 12.5 mmol) at 25° C. and then the mixture was stirred for 16 h at 80° C. LCMS showed 14% of starting material was remained and 80% of desired product was detected (14%, Rt=0.244 min, [M+H]⁺=208.3 at 220 nm). Mg (chips) (7.79 eq, 150 mg, 6.25 mmol) was added to the reaction and then the mixture was stirred at 80° C. for another 12 h. LCMS showed 97% of desired product was detected (97%, Rt=0.244 min, [M+H]⁺=208.3 at 220 nm). The mixture was filtered and the filter cake was washed with MeOH (30 mL*3), the combine organic layers was concentrated under reduced pressure to give the (2R,6S)-2-cyclopropyl-6-(1-methylpyrazol-4-yl)morpholine (560 mg, 2.70 mmol, 336.75% yield) as white solid and the product was used to the next step directly.

Step 8: To a solution of (2R,6S)-2-cyclopropyl-6-(1-methylpyrazol-4-yl)morpholine (2.37 eq, 480 mg, 2.32 mmol) in DMSO (6 mL) was added 2-chloro-4-(2,4-difluorophenyl)-6,7-dimethyl-pteridine (1.00 eq, 300 mg, 0.978 mmol) and DIEA (3.00 eq, 0.51 mL, 2.93 mmol). The mixture was stirred at 100° C. for 1 h. LCMS showed starting material was consumed completely and 70% of desired product was detected (70%, Rt=0.985 min, [M+H]⁺=478.3 at 220 nm). The mixture was quenched by 100 mL H₂O, extracted with EA (150 mL*3), the combine organic layers was dried over anhydrous Na₂SO₄, filtered and concentrated under reduced pressure to give the (2R,6S)-2-cyclopropyl-4-[4-(2,4-difluorophenyl)-6,7-dimethyl-pteridin-2-yl]-6-(1-methylpyrazol-4-yl)morpholine (250 mg, 0.524 mmol, 53.52% yield) as red solid, LCMS, [M+H]⁺=478.3; purity=96% (220 nm). Retention time=0.998 min. 1H NMR (400 MHz, CDCl₃) δ=7.72 (q, J=7.8 Hz, 1H), 7.55 (s, 1H), 7.46 (s, 1H), 7.05 (br t, J=7.9 Hz, 1H), 6.98 (br t, J=9.4 Hz, 1H), 5.06 (br d, J=9.8 Hz, 2H), 4.55 (dd, J=2.1, 10.8 Hz, 1H), 3.90 (s, 3H), 3.13-2.96 (m, 3H), 2.72 (s, 3H), 2.60 (s, 3H), 1.06-0.93 (m, 1H), 0.67-0.54 (m, 2H), 0.52-0.34 (m, 2H). SFC showed 2 peaks (ratio was 1:1) as a cis-mixture.

Step 9: The product was purified by SFC (“Column: Chiralpak IC-3 50×4.6 mm I.D., 3 um, Mobile phase: Phase A for CO₂, and Phase B for EtOH (0.05% DEA); Gradient elution: 40% EtOH (0.05% DEA) in CO₂, Flow rate: 3 mL/min; Detector: PDA, Column Temp: 35° C.; Back Pressure: 100 Bar”) and lyophilized to give the (2R,6S)-2-cyclopropyl-4-[4-(2,4-difluorophenyl)-6,7-dimethyl-pteridin-2-yl]-6-(1-methylpyrazol-4-yl)morpholine (93 mg, 0.192 mmol, 37.16% yield) as yellow solid. (2S,6R)-2-cyclopropyl-4-[4-(2,4-difluorophenyl)-6,7-dimethyl-pteridin-2-yl]-6-(1-methylpyrazol-4-yl)morpholine (103 mg, 0.206 mmol, 39.87% yield) as brown solid, LCMS: [M+H]⁺=478.3; purity=96% (220 nm). Retention time=0.956 min. 1H NMR (400 MHz, CDCl₃) δ=7.72 (q, J=7.6 Hz, 1H), 7.56 (s, 1H), 7.46 (s, 1H), 7.05 (br t, J=8.3 Hz, 1H), 7.01-6.92 (m, 1H), 5.19-4.97 (m, 2H), 4.55 (dd, J=2.5, 10.7 Hz, 1H), 3.90 (s, 3H), 3.14-2.96 (m, 3H), 2.72 (s, 3H), 2.60 (s, 3H), 1.08-0.95 (m, 1H), 0.67-0.54 (m, 2H), 0.53-0.33 (m, 2H).SFC showed ee 99%. LCMS: [M+H]⁺=478.3; purity=96% (220 nm). Retention time=0.964 min. 1H NMR (400 MHz, CDCl3) δ=7.76-7.68 (m, 1H), 7.56 (s, 1H), 7.46 (s, 1H), 7.09-7.02 (m, 1H), 6.98 (br t, J=9.5 Hz, 1H), 5.17-4.97 (m, 2H), 4.55 (dd, J=2.5, 10.7 Hz, 1H), 3.90 (s, 3H), 3.17-2.96 (m, 3H), 2.72 (s, 3H), 2.60 (s, 3H), 1.09-0.95 (m, 1H), 0.66-0.54 (m, 2H), 0.51-0.31 (m, 2H) SFC showed ee 99.3%.

### Synthesis of Compound I-1686

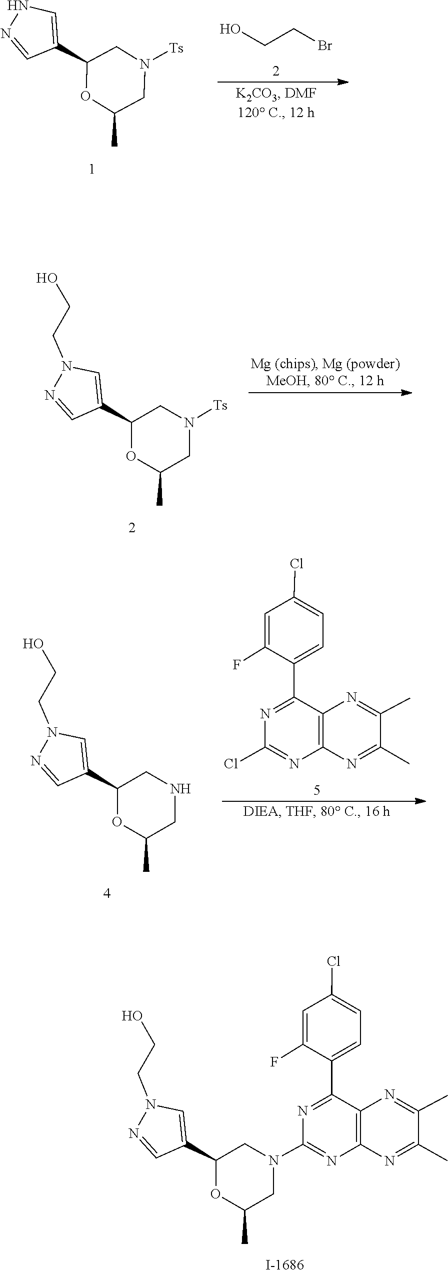

Step 1: A solution of (2R,6S)-2-methyl-4-(p-tolylsulfonyl)-6-(1H-pyrazol-4-yl)morpholine (1.00 eq, 450 mg, 1.40 mmol), 2-bromoethanol (1.70 eq, 0.17 mL, 2.38 mmol) and K₂CO₃ (2.00 eq, 387 mg, 2.80 mmol) in DMF (15 mL) was stirred at 120° C. for 4 h. LCMS (5-95AB/1.5 min): RT=0.835 min, 366.1=[M+H]⁺, ESI⁺ showed 85% of desired product. The reaction mixture was partitioned between ethyl acetate (50 mL*2) and water (80 mL). The combined organic layers were dried over Na₂SO₄, filtered and concentrated under reduced pressure to give a residue. The crude product was purified by column chromatography on silica gel chromatography (ethyl acetate, the desired product R_{f}=0.2, showed by phosphomolybdic acid) to give 2-(4-((2S,6R)-6-methyl-4-tosylmorpholin-2-yl)-1H-pyrazol-1-yl)ethan-1-ol (430 mg, 1.18 mmol, 84.04% yield) as light white solid. LCMS: (M+H)=366.1; purity=78% (220 nm); retention time=0.524 min. ¹H NMR (400 MHz, CDCl3) δ=7.64 (d, J=8.3 Hz, 2H), 7.49-7.38 (m, 2H), 7.35 (d, J=8.0 Hz, 2H), 4.66 (dd, J=2.5, 10.5 Hz, 1H), 4.28-4.17 (m, 2H), 4.06-3.90 (m, 2H), 3.90-3.80 (m, 1H), 3.74 (br d, J=11.4 Hz, 1H), 3.63 (br d, J=11.4 Hz, 1H), 2.45 (s, 3H), 2.23 (t, J=11.0 Hz, 1H), 2.08 (s, 1H), 1.21-1.15 (m, 3H)

Step 2: To the mixture of 2-[4-[(2S,6R)-6-methyl-4-(p-tolylsulfonyl)morpholin-2-yl]pyrazol-1-yl]ethanol (1.00 eq, 430 mg, 1.18 mmol) in Methanol (8 mL) was added Mg (10.0 eq, 282 mg, 11.8 mmol) (chips) and Mg (10.0 eq, 282 mg, 11.8 mmol)(powder) at 25° C. and the reaction mixture was stirred for 12 hours at 80° C. LCMS (5-95AB/1.5 min): RT=0.221 min, 212.1=[M+H]⁺, ESI⁺ showed 42.7% of desired product. The reaction mixture was filtered through celite, the filtrate was evaporated under reduced pressure to give the crude product 2-(4-((2S,6R)-6-methylmorpholin-2-yl)-1H-pyrazol-1-yl)ethan-1-ol (450 mg, 1.17 mmol, 99.74% yield) as a light-yellow solid. LCMS: (M+H)=212.2; purity=80% (220 nm); retention time=0.221 min. ¹H NMR (400 MHz, DMSO) δ=7.34 (d, J=7.9 Hz, 1H), 7.11 (d, J=7.7 Hz, 1H), 4.12-3.96 (m, 2H), 3.74-3.62 (m, 2H), 3.42-3.31 (m, 2H), 3.22-3.14 (m, 2H), 2.89 (s, 1H), 2.73 (s, 1H), 2.34-2.17 (m, 2H), 1.26-0.97 (m, 3H)

Step 3: To a solution of 4-methylbenzenesulfonic acid;2-[4-[(2S,6R)-6-methylmorpholin-2-yl]pyrazol-1-yl]ethanol (1.20 eq, 142 mg, 0.371 mmol) and 2-chloro-4-(4-chloro-2-fluoro-phenyl)-6,7-dimethyl-pteridine (1.00 eq, 100 mg, 0.309 mmol) in THF (20 mL) was added DIEA (5.00 eq, 0.26 mL, 1.55 mmol), then the mixture was stirred at 80° C. for 8 h. LCMS showed the starting material was still remained. The reaction mixture was added 4-methylbenzenesulfonic acid;2-[4-[(2S,6R)-6-methylmorpholin-2-yl]pyrazol-1-yl]ethanol (0.843 eq, 100 mg, 0.261 mmol), then the mixture was stirred at 80° C. for 12 h. LCMS (5-95AB/1.5 min): RT=0.940 min, 498.2=[M+H]⁺, ESI⁺ showed 72% of desired product. The reaction mixture was partitioned between ethyl acetate (50 mL*2) and water (80 mL). The combined organic layers were dried over Na₂SO₄, filtered and concentrated under reduced pressure to give a residue. The crude product was purified by prep-TLC (DCM/MeOH=10:1, the desired product R_{f}=0.2, showed by 254 nm) twice to give 2-(4-((2S,6R)-4-(4-(4-chloro-2-fluorophenyl)-6,7-dimethylpteridin-2-yl)-6-methylmorpholin-2-yl)-1H-pyrazol-1-yl)ethan-1-ol (12 mg, 0.0240 mmol, 7.76% yield) as yellow solid, checked by LCMS: (M+H)=498.2; purity=97% (220 nm); retention time=0.900 min ¹H NMR (400 MHz, CDCl3) δ=7.68-7.60 (m, 2H), 7.54 (br s, 1H), 7.31 (br d, J=8.1 Hz, 1H), 7.25 (br s, 1H), 5.13-4.95 (m, 2H), 4.63 (br d, J=10.1 Hz, 1H), 4.24 (br s, 2H), 4.00 (br s, 2H), 3.84 (br s, 1H), 3.09 (br s, 1H), 2.87 (br d, J=11.5 Hz, 1H), 2.71 (s, 3H), 2.59 (s, 3H), 1.33 (br d, J=6.0 Hz, 4H).

### Synthesis of Compound I-1691

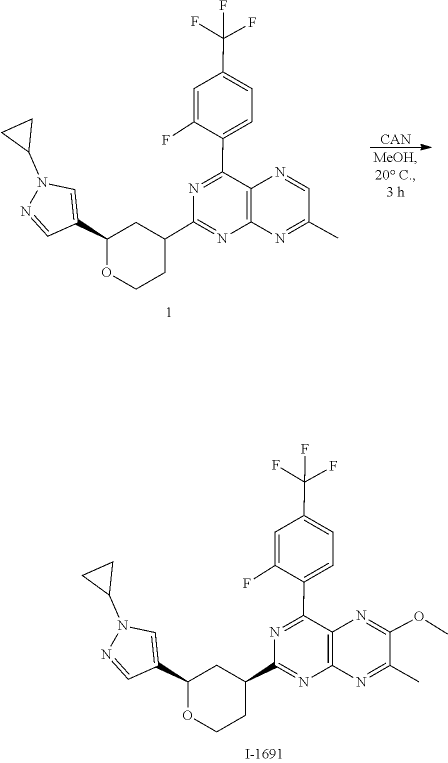 [SMILES: Cc1cnc2c(-c3ccc(C(F)(F)F)cc3F)nc(C3CCO[C@@H](c4cnn(C5CC5)c4)C3)nc2n1 ; "CAN" ; COc1nc2c(-c3ccc(C(F)(F)F)cc3F)nc([C@H]3CCO[C@@H](c4cnn(C5CC5)c4)C3)nc2nc1C]

To a solution of 2-[(2R,4S)-2-(1-cyclopropylpyrazol-4-yl)tetrahydropyran-4-yl]-4-[2-fluoro-4-(trifluoromethyl)phenyl]-7-methyl-pteridine (1.00 eq, 90 mg, 0.181 mmol) in Methanol (4 mL) was added CAN (1.50 eq, 160 mg, 0.292 mmol) by portions and then stirred for 3 h at 20° C. LCMS (5-95AB/1.5 min): RT=1.007 min, 529.0=[M+H]⁺, ESI⁺ showed 42.7% of desired product. The mixture was stirred for 2 h at 20° C. LCMS (5-95AB/1.5 min): RT=0.707 min, 529.2=[M+H]⁺, ESI⁺ showed 25% of desired product. The reaction mixture was partitioned between DCM (50 mL*2) and Na₂SO₃ (aq, 50 mL). The combined organic layers were dried over Na₂SO₄, filtered and concentrated under reduced pressure. The crude product was purified by prep-HPLC (Phenomenex luna C18 150*25 mm * 10 um; mobile phase: [water (0.1% FA)-ACN]; B %: 60%-90%, 12 min) and lyophilized to give 2-((2R,4S)-2-(1-cyclopropyl-1H-pyrazol-4-yl)tetrahydro-2H-pyran-4-yl)-4-(2-fluoro-4-(trifluoromethyl)phenyl)-6-methoxy-7-methylpteridine (15 mg, 0.0272 mmol, 25.3% yield) as white solid, checked by LCMS: (M+H)⁺=529.2; purity=99% (220 nm); Retention time=1.002 min. ¹H NMR (400 MHz, CDCl₃) δ=7.87 (t, J=7.3 Hz, 1H), 7.62 (d, J=8.0 Hz, 1H), 7.54-7.48 (m, 3H), 4.56 (dd, J=1.9, 11.4 Hz, 1H), 4.30-4.24 (m, 1H), 4.01 (s, 3H), 3.85-3.77 (m, 1H), 3.59-3.49 (m, 2H), 2.77 (s, 3H), 2.43 (br d, J=13.4 Hz, 1H), 2.26-2.21 (m, 1H), 2.20-2.16 (m, 2H), 1.12-1.07 (m, 2H), 1.02-0.96 (m, 2H).

### Synthesis of Compounds 1-1696, 1-1698, 1-1699 and I-1700

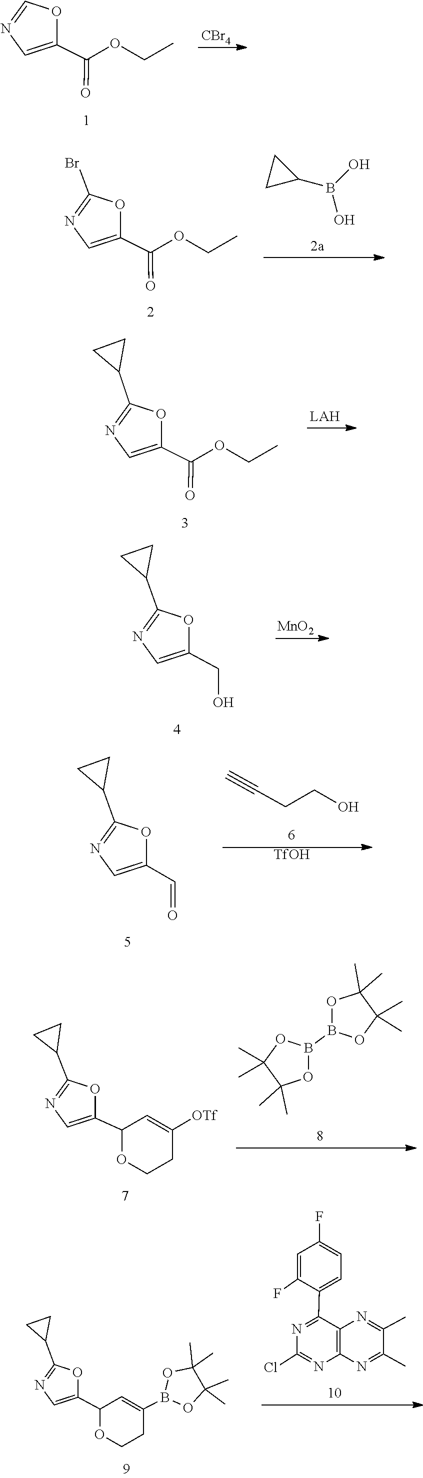

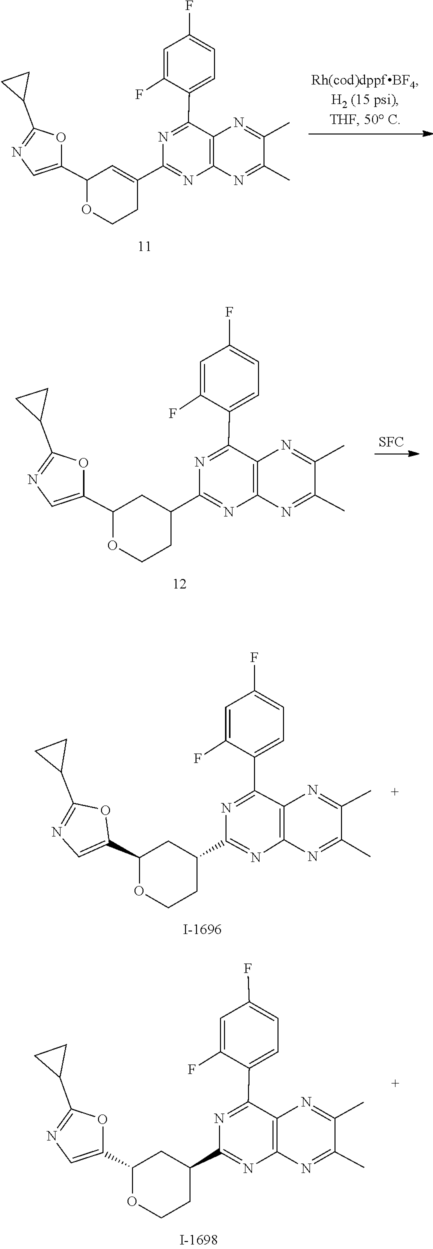

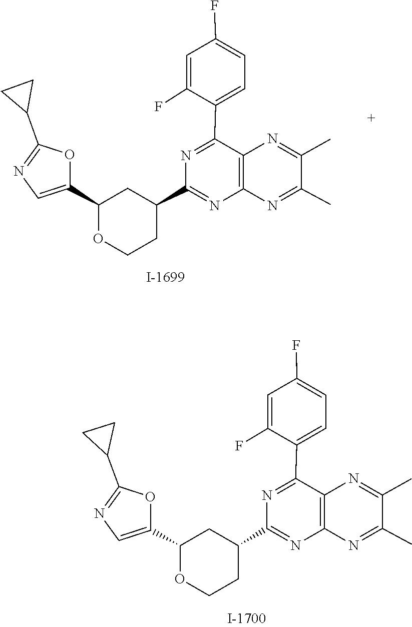 [SMILES: Cc1nc2nc([C@H]3CCO[C@@H](c4cnc(C5CC5)o4)C3)nc(-c3ccc(F)cc3F)c2nc1C ; Cc1nc2nc([C@@H]3CCO[C@H](c4cnc(C5CC5)o4)C3)nc(-c3ccc(F)cc3F)c2nc1C]

Step 1: To a solution of ethyl oxazole-5-carboxylate (1.00 eq, 10.00 g, 70.9 mmol) in THF (75 mL) was added LiHMDS (1.10 eq, 78 mL, 77.9 mmol) dropwise at −78° C. under N₂ atmosphere and stirred for 20 min. Then a solution of CBr₄ (1.40 eq, 3.29 g, 99.2 mmol) in THF (15 mL) was added slowly at −78° C. Then the reaction mixture was stirred at −78° C. for 2 hours under N₂ atmosphere and warmed to 20° C. for 12 h. TLC (PE:EtOAc=2:1; UV) showed several spots formed and desired spot found (Rf=0.4). The mixture was added water (100 mL) and extracted with EtOAc (150 mL×3). The organic phase was concentrated under vacuum to give a crude. The crude was purified by flash column (PE:EtOAc=2:1, Rf=0.4). Ethyl 2-bromooxazole-5-carboxylate (3200 mg, 14.5 mmol, 20% yield) was obtained as a light-yellow oil. ¹H NMR (400 MHz, chloroform-d) 6=7.60-7.47 (m, 1H), 4.30-4.19 (m, 2H), 1.27-1.20 (m, 3H)

Step 2: A mixture of ethyl 2-bromooxazole-5-carboxylate (1.00 eq, 3200 mg, 14.5 mmol), cyclopropylboronic acid (1.60 eq, 1999 mg, 23.3 mmol), K₃PO₄ (3.00 eq, 9250 mg, 43.6 mmol) in Toluene (32.5 mL) and Water (3.9 mL) was added Pd(dppf)Cl₂ DCM (0.0500 eq, 532 mg, 0.727 mmol). The mixture was degassed with N₂ for 3 times and stirred at 120° C. for 16 h. LCMS showed a peak with desired MS (44%, MS: 182 [M+H]⁺, RT=0.584 min) was detected. The mixture was concentrated under vacuum to give a crude. The crude was purified by flash chromatography (PE:EtOAc=2:1; UV, Rf=0.4) to give ethyl 2-cyclopropyloxazole-5-carboxylate (1300 mg, 7.01 mmol, 48.20% yield) as yellow oil. LCMS: (M+H)=182.1; purity=97.7% (UV 220 nm); Retention time=0.587 min. 11H NMR (400 MHz, chloroform-d) 6=7.47 (s, 1H), 4.23 (q, J=7.2 Hz, 2H), 2.07-1.97 (m, 1H), 1.24 (t, J=7.2 Hz, 3H), 1.09-1.03 (m, 2H), 1.03-0.97 (m, 2H).

Step 3: A mixture of ethyl 2-cyclopropyloxazole-5-carboxylate (1.00 eq, 1300 mg, 7.17 mmol) in THF (60 mL) was cooled to 0° C. and added LiAlH₄ (2.00 eq, 545 mg, 14.3 mmol) slowly. The mixture was stirred at 20° C. for 3 h. LCMS showed the starting material was consumed completely and a major peak with desired MS (98.7%, MS: 140 [M+H]⁺, RT=0.322 min) was detected. The mixture was added 545 mg water slowly at 0° C. The mixture was filtered and the filtrate was concentrated under vacuum to give (2-cyclopropyloxazol-5-yl)methanol (760 mg, 5.46 mmol, 76.12% yield) was obtained as a light-yellow oil. LCMS: (M+H)⁺=140.1; purity=97.2% (UV 220 nm); Retention time=0.270 min. ¹H NMR (400 MHz, chloroform-d) 6=6.82 (s, 1H), 4.60 (br s, 2H), 2.14-1.98 (m, 1H), 1.13-0.96 (m, 4H).

Step 4: A mixture of (2-cyclopropyloxazol-5-yl)methanol (1.00 eq, 360 mg, 2.59 mmol) in DCE (8 mL) was added MnO₂ (10.0 eq, 2249 mg, 25.9 mmol). The mixture was stirred at 60° C. for 2 h. LCMS showed a major peak with desired MS (57%, MS: 138 [M+H]⁺, RT=0.462 min). The mixture was filtered and the filtrate was concentrated under vacuum to give 2-cyclopropyloxazole-5-carbaldehyde (320 mg, 2.33 mmol, 90.19% yield) as a light-yellow oil. LCMS: (M+H)=138.1; purity=83.9% (UV 220 nm); Retention time=0.645 min. ¹H NMR (400 MHz, chloroform-d) 6=9.68-9.61 (m, 1H), 7.79-7.63 (m, 1H), 2.24-2.12 (m, 1H), 1.30-1.14 (m, 5H).

Step 5: A mixture of 2-cyclopropyloxazole-5-carbaldehyde (1.00 eq, 410 mg, 2.99 mmol) and 3-butyn-1-ol (1.50 eq, 0.34 mL, 4.48 mmol) in DCM (8 mL) was cooled to 20° C. The mixture was added trifluoromethanesulfonic acid (2.40 eq, 0.64 mL, 7.18 mmol) slowly and stirred at 20° C. for 3 h. Then trifluoromethanesulfonic acid (2.40 eq, 0.64 mL, 7.18 mmol) was added and the mixture was stirred at 20° C. for 3 h. LCMS showed a major peak with desired MS (81%, MS: 340 [M+H]⁺, RT=0.737 min) was found. The mixture was concentrated under vacuum and purified by reversed phase-HPLC (FA, FA in water: ACN=100% to 0%, 220 and 254 nm) and lyophilized to give [6-(2-cyclopropyloxazol-5-yl)-3,6-dihydro-2H-pyran-4-yl] trifluoromethanesulfonate (330 mg, 0.973 mmol, 32.53% yield) as yellow oil. LCMS: (M+H)⁺=340; purity=77.3% (UV 220 nm); Retention time=0.739 min. ¹H NMR (400 MHz, chloroform-d) δ=6.86 (s, 1H), 5.94-5.90 (m, 1H), 5.40-5.35 (m, 1H), 3.93-3.87 (m, 2H), 2.67-2.54 (m, 3H), 2.49-2.39 (m, 2H), 2.12-2.03 (m, 2H), 1.13-1.03 (m, 5H).

Step 6: To a colorless mixture of B₂pin₂ (1.30 eq., 404 mg, 1.59 mmol) in 1,4-dioxane (7 mL) was added [6-(2-cyclopropyloxazol-5-yl)-3,6-dihydro-2H-pyran-4-yl] trifluoromethanesulfonate (1.00 eq., 415 mg, 1.22 mmol), potassium acetate (3.00 eq, 360 mg, 3.67 mmol), Pd(dppf)Cl₂ DCM (0.1000 eq, 89 mg, 0.122 mmol), then the brown mixture was stirred at 90° C. for 12 h under N₂ atmosphere to give a black solution. LCMS showed 44% desired MS found (44%, MS: 318 [M+H]⁺, RT=0.742 min). The mixture was concentrated under vacuum to give a crude. The crude was purified by flash column (PE:EtOAc=10:1 to 1:1; ammonium tetramolybdate, Rf=0.3) and concentrated under vacuum to give 2-cyclopropyl-5-[4-(4,4,5,5-tetramethyl-1,3,2-dioxaborolan-2-yl)-3,6-dihydro-2H-pyran-6-yl]oxazole (330 mg, 0.739 mmol, 60.39% yield) as yellow oil. LCMS: (M+H)=318; purity=71.6% (UV 220 nm); retention time=0.736 min.

Step 7: To a solution of 2-cyclopropyl-5-[4-(4,4,5,5-tetramethyl-1,3,2-dioxaborolan-2-yl)-3,6-dihydro-2H-pyran-6-yl]oxazole (1.00 eq, 290 mg, 0.914 mmol),2-chloro-4-(2,4-difluorophenyl)-6,7-dimethyl-pteridine (1.00 eq, 280 mg, 0.913 mmol) and K₂CO₃ (2.00 eq, 153 mg, 1.83 mmol) in 1,4-dioxane (7 mL) and water (1.4 mL), [1,1′-Bis(diphenylphosphino)ferrocene]dichloropalladium(II) (0.1 eq, 67 mg, 0.0913 mmol) was added and purged with N₂ for 3 times, the reaction solution was stirred at 80° C. for 2 hrs showed the reactant was consumed and ˜83% of desired mass was detected (LCMS (M+H)+=462.2; Retention time=0.789 min). The mixture was concentrated under vacuum and was purified by flash column (PE:EtOAc=1:1, UV, Rf=0.2) to give 2-cyclopropyl-5-[4-[4-(2,4-difluorophenyl)-6,7-dimethyl-pteridin-2-yl]-3,6-dihydro-2H-pyran-6-yl]oxazole (190 mg, 0.395 mmol, 43% yield) as a yellow solid. LCMS: (M+H)=462; purity=96.9% (UV 220 nm); retention time=0.977 min. ¹H NMR (400 MHz, chloroform-d) 6=7.84-7.75 (m, 1H), 7.65-7.59 (m, 1H), 7.13-7.05 (m, 1H), 7.04-6.96 (m, 1H), 6.90-6.85 (m, 1H), 5.51 (br d, J=2.6 Hz, 1H), 4.06-3.95 (m, 2H), 3.09-2.91 (m, 2H), 2.85 (s, 3H), 2.73 (s, 3H), 2.05 (br s, 1H), 1.13-1.06 (m, 2H), 1.06-0.99 (m, 2H).

Step 8: A solution of 2-cyclopropyl-5-[4-[4-(2,4-difluorophenyl)-6,7-dimethyl-pteridin-2-yl]-3,6-dihydro-2H-pyran-6-yl]oxazole (1.00 eq, 50 mg, 0.108 mmol) in THF (10 mL) was added 1,1′-bis(di-i-propylphosphino) ferrocene (1,5-cyclooctadiene)rhodium (i) tetra-fluoroborate (0.203 eq, 5.0 mg, 0.0220 mmol) under N₂ atmosphere, the mixture was purged by H₂ for 3 times, then stirred at 50° C. for 12 h under H₂ atmosphere (balloon, 15 psi). LCMS showed starting material consumed completely and a major peak with desired MS (74%, MS: 464 [M+H]⁺, RT=0.763 min). The mixture was concentrated under vacuum to give a crude. The crude was purified by reversed phase-HPLC (FA, FA in water: ACN=100% to 0%, UV 220 and 254 nm) and lyophilized to give 20 mg product 2-cyclopropyl-5-[4-[4-(2,4-difluorophenyl)-6,7-dimethyl-pteridin-2-yl]tetrahydropyran-2-yl]oxazole (20 mg, 0.0411 mmol, 37.95% yield) as yellow solid. 105 mg of product showed four peaks. Condition: 0.1% NH₃H₂O EtOH; Column: DAICEL CHIRALCEL OJ-H (250 mm*30 mm, 5 um); Flowrate (mL/min): 50, Column: Chiralcel OJ-3 50×4.6 mm I.D., 3 um; mobile phase: phase A for CO₂, and Phase B for MeOH (0.05% DEA); gradient elution: B in A from 5% to 40%; flow rate: 3 mL/min; detector: DAD; column temp: 35° C.; Back Pressure: 100 Bar).

Step 9: 105 mg product was sent for SFC separation (Condition: 0.1% NH₃H2O EtOH; Column: DAICEL CHIRALCEL OJ-H (250 mm*30 mm, 5 um); Flowrate (mL/min): 50) and four peaks were obtained. Peak 1 in SFC-2-cyclopropyl-5-[(2R,4R)-4-[4-(2,4-difluorophenyl)-6,7-dimethyl-pteridin-2-yl]tetrahydropyran-2-yl]oxazole (17 mg, 0.0344 mmol, 31.78% yield) as off-white solid. LCMS (M+H)⁺=464.3; purity=95% (220 nm); retention time=0.753 min. ¹H NMR (400 MHz, chloroform-d) 6=7.81-7.72 (m, 1H), 7.09 (dt, J=2.2, 8.3 Hz, 1H), 7.00 (dt, J=2.4, 9.5 Hz, 1H), 5.07-4.96 (m, 1H), 4.00-3.83 (m, 2H), 3.81-3.70 (m, 1H), 2.87-2.82 (m, 3H), 2.76-2.73 (m, 3H), 2.69 (br s, 1H), 2.53-2.35 (m, 2H), 2.30-2.17 (m, 1H), 2.12-2.00 (m, 1H), 1.11-0.97 (m, 4H). Peak 2 in SFC-2-cyclopropyl-5-[(2S,4S)-4-[4-(2,4-difluorophenyl)-6,7-dimethyl-pteridin-2-yl]tetrahydropyran-2-yl]oxazole (15 mg, 0.0316 mmol, 29.18% yield) as a light-yellow solid. LCMS (M+H)⁺=464.3; purity=96.4% (220 nm); Retention time=0.751 min. ¹H NMR (400 MHz, chloroform-d) 6=7.84-7.72 (m, 1H), 7.13-7.05 (m, 1H), 7.04-6.96 (m, 1H), 6.96-6.89 (m, 1H), 5.09-4.94 (m, 1H), 3.99-3.84 (m, 2H), 3.82-3.70 (m, 1H), 2.85 (s, 3H), 2.79-2.70 (m, 4H), 2.52-2.36 (m, 2H), 2.30-2.18 (m, 1H), 2.14-2.03 (m, 1H), 1.13-1.07 (m, 2H), 1.06-0.99 (m, 2H). Peak 3 in SFC-2-cyclopropyl-5-[(2R,4S)-4-[4-(2,4-difluorophenyl)-6,7-dimethyl-pteridin-2-yl]tetrahydropyran-2-yl]oxazole (11 mg, 0.0230 mmol, 21.25% yield) as a light-yellow solid. LCMS (M+H)⁺=464.2; purity=97.9% (220 nm); Retention time=0.743 min. ¹H NMR (400 MHz, chloroform-d) 6=7.82-7.70 (m, 1H), 7.14-7.05 (m, 1H), 7.05-6.96 (m, 1H), 6.92-6.86 (m, 1H), 4.66-4.56 (m, 1H), 4.35-4.24 (m, 1H), 3.91-3.76 (m, 1H), 3.60-3.46 (m, 1H), 2.85 (s, 3H), 2.73 (s, 3H), 2.51-2.33 (m, 2H), 2.26-2.16 (m, 2H), 2.10-1.98 (m, 1H), 1.10-1.04 (m, 2H), 1.03-0.96 (m, 2H). Peak 4 in SFC-2-cyclopropyl-5-[(2S,4R)-4-[4-(2,4-difluorophenyl)-6,7-dimethyl-pteridin-2-yl]tetrahydropyran-2-yl]oxazole (16 mg, 0.0325 mmol, 30.01%% yield) as off-white solid. LCMS (M+H)⁺=464.2; purity=96.4% (220 nm); Retention time=0.746 min. ¹H NMR (400 MHz, chloroform-d) 6=7.80-7.72 (m, 1H), 7.13-7.05 (m, 1H), 7.04-6.96 (m, 1H), 6.91-6.86 (m, 1H), 4.68-4.52 (m, 1H), 4.34-4.23 (m, 1H), 3.88-3.76 (m, 1H), 3.60-3.43 (m, 1H), 2.85 (s, 3H), 2.75-2.69 (m, 3H), 2.49-2.31 (m, 2H), 2.25-2.15 (m, 2H), 2.09-1.99 (m, 1H), 1.09-1.04 (m, 2H), 1.03-0.97 (m, 2H). (Cis- pairs of enantiomers were distinguished from trans- pairs of enantiomers by NMR).

### I. Synthesis of Compounds I-1701 and I-1702

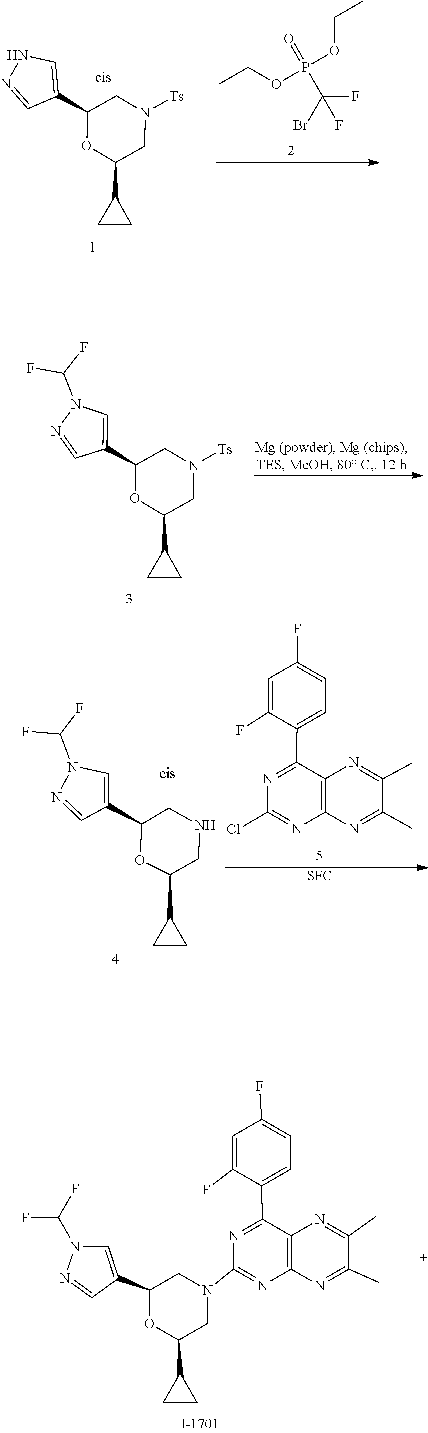

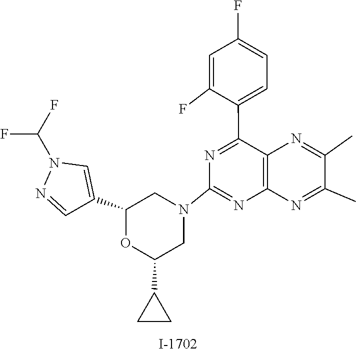 [SMILES: Cc1nc2nc(N3C[C@@H](c4cnn(C(F)F)c4)O[C@@H](C4CC4)C3)nc(-c3ccc(F)cc3F)c2nc1C]

Step 1: To a solution of 2-cyclopropyl-4-(p-tolylsulfonyl)-6-(1H-pyrazol-4-yl)morpholine (1.00 eq, 600 mg, 1.73 mmol) and 1-[[bromo(difluoro)methyl]-ethoxy-phosphoryl]oxyethane (1.50 eq, 692 mg, 2.59 mmol) in MeCN (14 mL) was added KF (2.00 eq, 201 mg, 3.45 mmol) at 20° C. The mixture was stirred at 40° C. for 3 h. LCMS showed ˜ 53% of desired product was detected (53%, Rt=0.971 min; [M+H]⁺=398.2 at 220 nm). The mixture was quenched by 80 mL ice water, extracted with EA (30 mL*3), the combine organic layers was dried over anhydrous Na₂SO₄, filtered and concentrated under reduced pressure to give the residue. The residue was purified with Flash Column (PE:EA=0˜20%, PE:EA=3:1, the desired product Rf=0.7) to give the 2-cyclopropyl-6-[1-(difluoromethyl)pyrazol-4-yl]-4-(p-tolylsulfonyl)morpholine (630 mg, 1.59 mmol, 91.79% yield) as colorless oil, checked by LCMS: [M+H]⁺=398.2, purity=99.5% (220 nm); Retention time=0.964 min. 1H NMR (400 MHz, chloroform-d) 6=7.76 (s, 1H), 7.65 (d, J=8.1 Hz, 2H), 7.59 (s, 1H), 7.37 (s, 2H), 7.18-6.96 (m, 1H), 4.61 (dd, J=1.9, 10.4 Hz, 1H), 4.37 (dt, J=5.1, 7.4 Hz, 2H), 3.81-3.69 (m, 2H), 3.07-2.98 (m, 1H), 2.45 (s, 3H), 0.86-0.74 (m, 1H), 0.56 (br dd, J=3.4, 7.3 Hz, 2H), 0.46-0.28 (m, 2H).

Step 2: To a solution of (2R,6S)-2-cyclopropyl-6-[1-(difluoromethyl)pyrazol-4-yl]-4-(p-tolylsulfonyl)morpholine (1.00 eq, 630 mg, 1.59 mmol) and Et₃SiH (46.5 eq, 12 mL, 73.7 mmol) in Methanol (30 mL) was added Mg (30.0 eq, 1141 mg, 47.6 mmol) (powder) and Mg (30.0 eq, 1141 mg, 47.6 mmol) (Chips) at 25° C. and the reaction mixture was stirred for 12 h at 80° C. under N₂ atmosphere. LCMS showed 47% of desired product was detected (47%, Rt=0.381 min; [M+H]⁺=244.2, UV 220 nm). The reaction mixture was cooled to room temperature. The mixture was filtered and the filter cake was washed with MeOH (30 mL*3), the combine organic layers was concentrated under reduced pressure to give crude (2R,6S)-2-cyclopropyl-6-[1-(difluoromethyl)pyrazol-4-yl]morpholine (730 mg, 3.00 mmol, 189.32% yield) as white solid.

Step 3: To a solution of 2-chloro-4-(2,4-difluorophenyl)-6,7-dimethyl-pteridine (1.00 eq, 159 mg, 0.520 mmol) in DMSO (6 mL) was added DIEA (3.00 eq, 0.27 mL, 1.56 mmol) and 2-cyclopropyl-6-[1-(difluoromethyl)pyrazol-4-yl]morpholine (1.00 eq, 240 mg, 0.520 mmol). The mixture was stirred at 100° C. for 1 h. LCMS showed 42% of desired product was detected (42%, Rt=1.046 min; [M+H]⁺=514.3 at 220 nm). The reaction was quenched by 60 mL H₂O, extracted with EA (30 mL*3), the combined organic layers was dried over anhydrous Na₂SO₄, filtered and concentrated under reduced pressure to give the residue. The residue was purified by Prep-TLC (PE:EA=2:1, the desired product Rf=0.6) to give the crude product (140 mg, 83% purity in LCMS). The crude product was purified again by Prep-TLC (PE:EA=2:1, the desired product Rf=0.6) to give the 2-cyclopropyl-6-[1-(difluoromethyl)pyrazol-4-yl]-4-[4-(2,4-difluorophenyl)-6,7-dimethyl-pteridin-2-yl]morpholine (110 mg, 0.214 mmol, 41% yield) as brown solid. The product was purified by SFC (DAICEL CHIRALPAK IC (250 mm*30 mm, 10 um), Mobile phase: Phase A for CO₂, and Phase B for EtOH (0.05% DEA); Gradient elution: EtOH (0.05% DEA) in CO₂ from 5% to 40%, Flow rate: 3 mL/min; Detector: PDA, Column Temp: 35C; Back Pressure: 100 Bar to give the (2S,6R)-2-cyclopropyl-6-[1-(difluoromethyl)pyrazol-4-yl]-4-[4-(2,4-difluorophenyl)-6,7-dimethyl-pteridin-2-yl]morpholine (34 mg, 0.0646 mmol, 12.42% yield) as yellow solid. (2R,6S)-2-cyclopropyl-6-[1-(difluoromethyl)pyrazol-4-yl]-4-[4-(2,4-difluorophenyl)-6,7-dimethyl-pteridin-2-yl]morpholine (30 mg, 0.0572 mmol, 11.01% yield) was obtained as yellow solid. LCMS: [M+H]⁺=514.4; purity=96.49% (220 nm); Retention time=0.937 min. HPLC: Retention time=2.555 min, 97.26% purity at 220 nm. SFC showed ee ˜ 100%. 1H NMR (400 MHz, chloroform-d) 6=7.90 (s, 1H), 7.74 (s, 1H), 7.73-7.67 (m, 1H), 7.33 (s, 1H), 7.18 (s, 1H), 7.08-7.02 (m, 1H), 6.98 (dt, J=2.3, 9.4 Hz, 1H), 5.08 (br s, 2H), 4.60 (dd, J=2.3, 10.8 Hz, 1H), 3.10-3.02 (m, 3H), 2.72 (s, 3H), 2.59 (s, 3H), 1.01 (br d, J=6.5 Hz, 1H), 0.67-0.56 (m, 2H), 0.54-0.45 (m, 1H), 0.44-0.34 (m, 1H). LCMS: [M+H]⁺=514.3; purity=98.60% (220 nm); Retention time=0.954 min. HPLC: retention time=2.534 min, 99.23% purity at 220 nm. SFC showed ee. 97%. 1H NMR (400 MHz, chloroform-d) 6=7.90 (s, 1H), 7.74 (s, 1H), 7.73-7.67 (m, 1H), 7.33 (s, 1H), 7.18 (s, 1H), 7.08-7.02 (m, 1H), 6.98 (dt, J=2.4, 9.4 Hz, 1H), 5.18-5.00 (m, 2H), 4.60 (dd, J=2.4, 10.9 Hz, 1H), 3.04 (br d, J=9.2 Hz, 3H), 2.72 (s, 3H), 2.60 (s, 3H), 1.01 (br d, J=6.2 Hz, 1H), 0.67-0.56 (m, 2H), 0.53-0.45 (m, 1H), 0.41 (br d, J=3.1 Hz, 1H).

### Synthesis of Compound I-1706

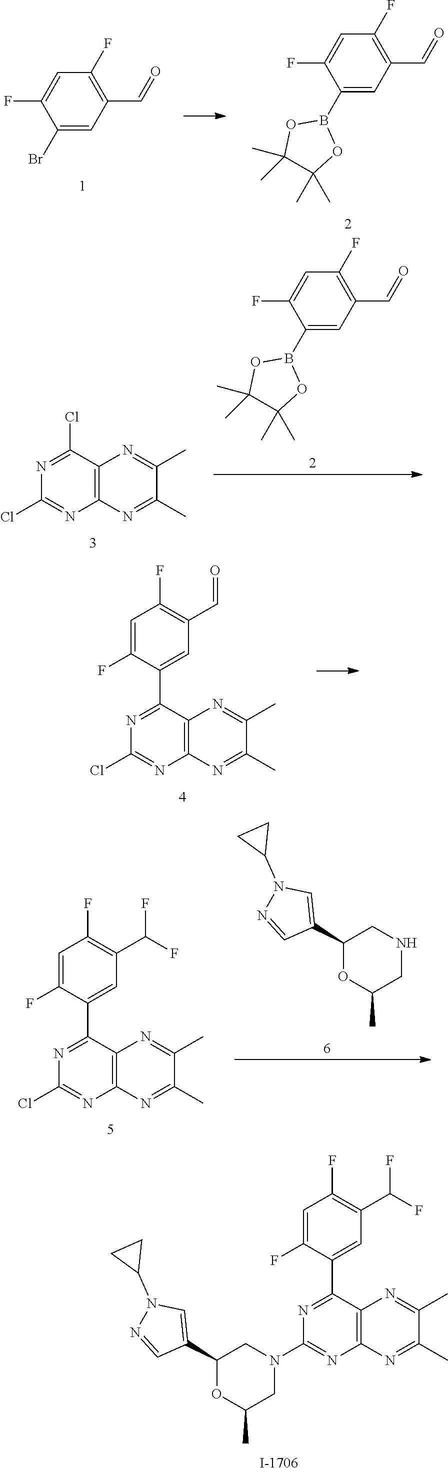

Step 1: To a solution of 5-bromo-2,4-difluoro-benzaldehyde (1.00 eq, 2000 mg, 9.05 mmol) in 1,4-Dioxane (15 mL) was added KOAc (2.50 eq, 2220 mg, 22.6 mmol) and 4,4,5,5-tetramethyl-2-(4,4,5,5-tetramethyl-1,3,2-dioxaborolan-2-yl)-1,3,2-dioxaborolane (1.50 eq, 3447 mg, 13.6 mmol), then Pd(dppf)Cl₂·CH₂Cl₂ (0.0500 eq, 367 mg, 0.452 mmol) was added to the mixture under N₂ and then the mixture was stirred for 16 h at 100° C. LCMS showed raw material was consumed and the major peak formed but without desired MS. TLC (PE/EA=10/1) showed raw material (Rf=0.6) consumed most and the new spot (Rf=0.4) formed. The reaction solution was poured into water (80 mL) and then was extracted with ethyl acetate (20 mL*3) and the organics washed with 20 mL saturated brine solution. The organics were then separated and dried (Na₂SO₄) before concentration to dryness. The crude was then purified by silica gel column (PE/EA=40/1) to give 2,4-difluoro-5-(4,4,5,5-tetramethyl-1,3,2-dioxaborolan-2-yl)benzaldehyde (1350 mg, 5.04 mmol, 55.65% yield) as light-yellow semi-solid. ¹H NMR (400 MHz, chloroform-d) δ ppm 1.37 (s, 13H) 6.88 (dd, J=10.58, 8.86 Hz, 1H) 8.35 (dd, J=8.56, 6.85 Hz, 1H) 10.27 (s, 1H).

Step 2: To a solution of 2,4-dichloro-6,7-dimethyl-pteridine (1.00 eq, 1068 mg, 4.66 mmol) and 2,4-difluoro-5-(4,4,5,5-tetramethyl-1,3,2-dioxaborolan-2-yl)benzaldehyde (1.00 eq, 1250 mg, 4.66 mmol) in toluene (20 mL) and water (2 mL) was added K₃PO₄ (3.00 eq, 2966 mg, 14.0 mmol) and PdCl₂ (amphos) (0.0500 eq, 165 mg, 0.233 mmol) under N₂ and then stirred for 16 h at 55° C. LCMS showed raw material was consumed and the major peak showed desired MS (M+H)⁺=335.0, purity=35.65%, uv=254 nm, Retention time=0.849 min. The combined reaction solutions were poured into water (50 mL), extracted with ethyl acetate (20 mL*3) and the organics washed with 10 mL saturated brine solution. The organics were then separated and dried (Na₂SO₄) before concentration to dryness. The crude product mixture was then purified by silica gel column (PE/EA=1/1) to give 5-(2-chloro-6,7-dimethyl-pteridin-4-yl)-2,4-difluoro-benzaldehyde (680 mg, 2.03 mmol, 43.57% yield) as brown solid. LCMS: (M+H)⁺=335.0, purity=81.04%, uv=220 nm, retention time=0.855 min.

Step 3: To a solution of 5-(2-chloro-6,7-dimethyl-pteridin-4-yl)-2,4-difluoro-benzaldehyde (1.00 eq, 580 mg, 1.73 mmol) in DCM (10 mL) was added BAST (5.00 eq, 1.6 mL, 8.66 mmol) at 0° C. and then stirred at 20° C. for 16 h. LCMS showed raw material was consumed and the major peak showed desired MS (M+H)⁺=357.0, purity=50.8%, uv=220 nm, Retention time=0.909 min. The reaction solution was poured into ice-NaHCO₃ (aq. 20 mL) slowly and then extracted with ethyl acetate (5 mL*2) and the organics washed with 5 mL saturated brine solution. The organics were then separated and dried (Na₂SO₄) before concentration to dryness. The crude was then purified by silica gel column (PE/EA=1/1) to give 2-chloro-4-[5-(difluoromethyl)-2,4-difluoro-phenyl]-6,7-dimethyl-pteridine (280 mg, 0.785 mmol, 45.30% yield) as brown solid. ¹H NMR (400 MHz, chloroform-d) δ ppm 2.75 (s, 3H) 2.85-2.90 (m, 3H) 6.79-7.14 (m, 1H) 6.79-6.82 (m, 1H) 6.94 (s, 1H) 8.07 (t, J=7.50 Hz, 1H).

Step 4: To a solution of 2-chloro-4-[5-(difluoromethyl)-2,4-difluoro-phenyl]-6,7-dimethyl-pteridine (1.00 eq, 100 mg, 0.280 mmol) in DMSO (2 mL) was added (2S,6R)-2-(1-cyclopropylpyrazol-4-yl)-6-methyl-morpholine (1.50 eq, 87 mg, 0.421 mmol) and DIEA (5.00 eq, 181 mg, 1.40 mmol) and then the mixture was stirred at 100° C. for 20 min. LCMS showed raw material was consumed completely and the major peak showed desired MS (M+H)⁺=528.2, purity=83.33%, uv=220 nm, Retention time=0.982 min. The crude reaction mixture solution was poured into water (20 mL), extracted with ethyl acetate (10 mL*2) and the organic phase washed with saturated brine solution (10 mL). Combined organic phases were separated and dried (Na₂SO₄) before filtration and concentration to dryness. The crude product mixture was then purified by prep-HPLC (Phenomenex C18 75*30 mm*3 um, water (FA)-ACN) and lyophilized to give (2S,6R)-2-(1-cyclopropylpyrazol-4-yl)-4-[4-[5-(difluoromethyl)-2,4-difluoro-phenyl]-6,7-dimethyl-pteridin-2-yl]-6-methyl-morpholine (49 mg, 0.0916 mmol, 32.67% yield) as yellow solid. LCMS: (M+H)⁺=528.2, purity=99.24%, uv=220 nm, retention time=0.980 min. ¹H NMR (400 MHz, chloroform-d) δ ppm 0.98-1.09 (m, 2H) 1.10-1.16 (m, 2H) 1.34 (d, J=6.25 Hz, 3H) 2.59 (s, 3H) 2.73 (s, 3H) 2.81-2.92 (m, 1H) 3.04-3.17 (m, 1H) 3.54-3.65 (m, 1H)_{3.77}-3.91 (m, 1H)_{4.55}-4.72 (m, 1H)_{4.86}-5.21 (m, 2H)_{6.78}-7.11 (m, 2H)_{7.27} (s, 1H) 7.55 (d, J=3.75 Hz, 2H) 7.94-8.03 (m, 1H).

### Synthesis of 1-1744

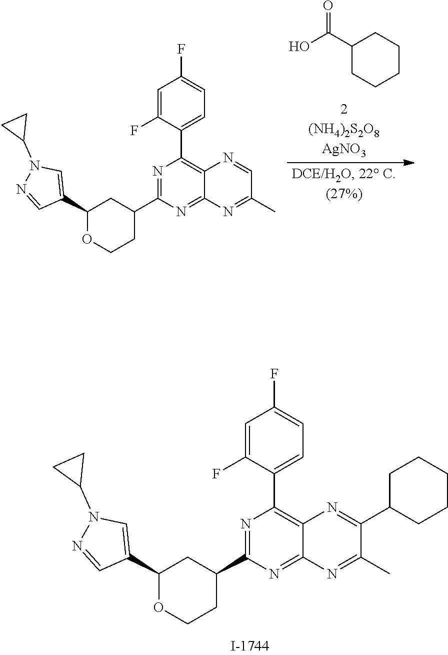 [SMILES: Cc1cnc2c(-c3ccc(F)cc3F)nc(C3CCO[C@@H](c4cnn(C5CC5)c4)C3)nc2n1 ; O=C(O)C1CCCCC1 ; Cc1nc2nc([C@H]3CCO[C@@H](c4cnn(C5CC5)c4)C3)nc(-c3ccc(F)cc3F)c2nc1C1CCCCC1]

A glass vial equipped with a Teflon-coated magnetic stirring bar was charged with 2-((2R)-2-(1-cyclopropyl-1H-pyrazol-4-yl)tetrahydro-2H-pyran-4-yl)-4-(2,4-difluorophenyl)-7-methylpteridine (prepared via Method 37)(1.0 equiv, 90 mg, 0.20 mmol), cyclohexanecarboxylic acid 2 (3.0 equiv, 76 mg, 0.60 mmol), ammonium persulfate (2.0 equiv, 92 mg, 0.40 mmol) and silver nitrate (0.1 equiv, 2 mg, 0.02 mmol). DCE (1.0 mL) and water (1.0 mL) were added, and the reaction mixture was stirred at 22° C., protected from light for 6 h. The reaction mixture was diluted with DCM and quenched with sat. NaHCO₃ (aq). The layers were separated, and the aqueous layer was extracted with DCM (×3). The combined organic layers were dried over Na₂SO₄, filtered and evaporated to dryness under reduced pressure. The residual crude material was purified by silica gel flash column chromatography (TeleDyne RediSep Gold column 24 g SiO₂) using an elution gradient of 0% to 10% MeOH in DCM to obtain the crude product as a yellow foam (66 mg). The product was further purified by prep HPLC (Gemini® 5 um NX-C18 110 Å, 100×30 mm column) using an elution gradient MeOH in 10 mM aqueous ammonium formate pH 3.8 (65-85%) to afford 6-cyclohexyl-2-((2R,4S)-2-(1-cyclopropyl-1H-pyrazol-4-yl)tetrahydro-2H-pyran-4-yl)-4-(2,4-difluorophenyl)-7-methylpteridine (29 mg, 0.05 mmol, 27% yield) as a light-yellow solid. LC-MS(ESI⁺): Tr=1.95 min; [M+H]⁺ 531.4 (obs). H NMR (CHCl₃-d, 400 MHz): ^{6H} 7.76-7.82 (1H, m), 7.49 (2H, s), 7.09 (1H, t, J=7.9 Hz), 7.00 (1H, t, J=9.6 Hz), 4.55 (1H, d, J=11.4 Hz), 4.25 (1H, d, J=11.6 Hz), 3.77-3.82 (1H, m), 3.49-3.58 (2H, m), 3.05-3.05 (1H, m), 2.88 (3H, d, J=2.5 Hz), 2.42 (1H, d, J=13.4 Hz), 2.17-2.26 (3H, m), 1.84-1.87 (4H, m), 1.77 (1H, d, J=13.0 Hz), 1.49-1.58 (3H, m), 1.37-1.47 (2H, m), 1.25-1.31 (1H, m), 1.07-1.08 (2H, m), 0.96-0.98 (2H, m). ¹⁹F NMR (CHCl₃-d, 376 MHz): δ_{F}−105.6 (1F, m), -106.5 (1F, m).

### II. Synthesis of Compounds I-1711 and I-1712

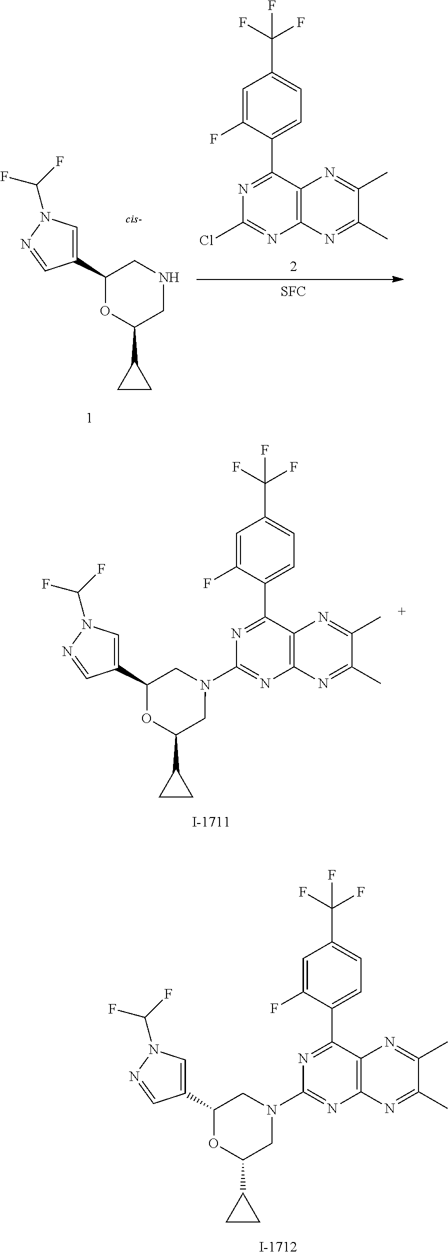 [SMILES: FC(F)n1cc([C@H]2CNC[C@@H](C3CC3)O2)cn1 ; Cc1nc2nc(Cl)nc(-c3ccc(C(F)(F)F)cc3F)c2nc1C ; "SFC" ; Cc1nc2nc(N3C[C@@H](C4CC4)O[C@@H](c4cnn(C(F)F)c4)C3)nc(-c3ccc(C(F)(F)F)cc3F)c2nc1C ; Cc1nc2nc(N3C[C@@H](c4cnn(C(F)F)c4)O[C@@H](C4CC4)C3)nc(-c3ccc(C(F)(F)F)cc3F)c2nc1C]

To a solution of (2R,6S)-2-cyclopropyl-6-[1-(difluoromethyl)pyrazol-4-yl]morpholine (1.49 eq, 183 mg, 0.751 mmol) in DMSO (6 mL) was added DIEA (3.00 eq, 0.26 mL, 1.51 mmol) and 2-chloro-4-[2-fluoro-4-(trifluoromethyl)phenyl]-6,7-dimethyl-pteridine (1.00 eq, 180 mg, 0.505 mmol). The mixture was stirred at 100° C. for 1 h. LCMS showed 51% of desired product was detected (51%, Rt=0.983 min; [M+H]⁺=564.3 at 220 nm). The reaction mixture was quenched by 60 mL H₂O, extracted with EA (30 mL*3), the combine organic layers was dried over anhydrous Na₂SO₄, filtered and concentrated under reduced pressure to give the residue. The residue was purified by prep-TLC (PE:EA=2:1, the desired product Rf=0.6) to give a crude product 150 mg (68% purity in LCMS). The crude product was purified again by prep-HPLC (flow: 25 mL/min; gradient: from 70-90% water (0.1% FA)-ACN over 10 min; column: Phenomenex luna C18 150*25 mm*5 um) and lyophilized to afford 2-cyclopropyl-6-[1-(difluoromethyl)pyrazol-4-yl]-4-[4-[2-fluoro-4-(trifluoromethyl)phenyl]-6,7-dimethyl-pteridin-2-yl]morpholine (88 mg, 0.156 mmol, 30.95% yield) as brown solid. The product was purified by SFC (Column: Chiralpak IC-3 50×4.6 mm I.D., 3 um Mobile phase: Phase A for CO₂, and Phase B for EtOH (0.05% DEA); Gradient elution: 40% EtOH (0.05% DEA) in CO₂, Flow rate: 3 mL/min; Detector: PDA, Column Temp: 35C; Back Pressure: 100 Bar to give (2R,6S)-2-cyclopropyl-6-[1-(difluoromethyl)pyrazol-4-yl]-4-[4-[2-fluoro-4-(trifluoromethyl)phenyl]-6,7-dimethyl-pteridin-2-yl]morpholine (17.16 mg, 0.0305 mmol, 6.03% yield) as yellow solid, and (2S,6R)-2-cyclopropyl-6-[1-(difluoromethyl)pyrazol-4-yl]-4-[4-[2-fluoro-4-(trifluoromethyl)phenyl]-6,7-dimethyl-pteridin-2-yl]morpholine (28 mg, 0.0486 mmol, 9.63% yield) as a brown solid, LCMS [M+H]⁺=564.2; purity=100.0% (220 nm); Retention time=0.979 min. HPLC: Retention time=2.717 min, 98.76% purity at 220 nm. SFC showed ee=100%. 1H NMR (400 MHz, chloroform-d) 6=7.90 (s, 1H), 7.85-7.77 (m, 1H), 7.74 (s, 1H), 7.59 (br d, J=8.0 Hz, 1H), 7.50 (br d, J=9.3 Hz, 1H), 7.33 (s, 1H), 7.18 (s, 1H), 7.03 (s, 1H), 5.08 (br dd, J=3.4, 7.9 Hz, 2H), 4.60 (dd, J=2.3, 10.8 Hz, 1H), 3.14-3.00 (m, 3H), 2.73 (s, 3H), 2.59 (s, 3H), 1.01 (br d, J=6.3 Hz, 1H), 0.62 (td, J=3.1, 7.9 Hz, 2H), 0.53-0.45 (m, 1H), 0.40 (br s, 1H) and a second peak, LCMS 564.2 [M+H]*; purity=97.1% (220 nm); Retention time=0.988 min. HPLC: Retention time=2.728 min, 98.74% purity at 220 nm. SFC showed ee=98%. 1H NMR (400 MHz, chloroform-d) 6=7.90 (s, 1H), 7.81 (br t, J=7.1 Hz, 1H), 7.74 (s, 1H), 7.59 (br d, J=8.0 Hz, 1H), 7.50 (br d, J=9.3 Hz, 1H), 7.33 (s, 1H), 7.18 (s, 1H), 7.03 (s, 1H), 5.21-4.95 (m, 2H), 4.60 (dd, J=2.0, 10.8 Hz, 1H), 3.06 (br s, 2H), 3.04 (s, 1H), 2.73 (s, 3H), 2.59 (s, 3H), 1.01 (br d, J=6.5 Hz, 1H), 0.66-0.56 (m, 2H), 0.48 (br dd, J=3.0, 4.3 Hz, 1H), 0.44-0.36 (m, 1H).

### Synthesis of compounds I-1716 and I-1729

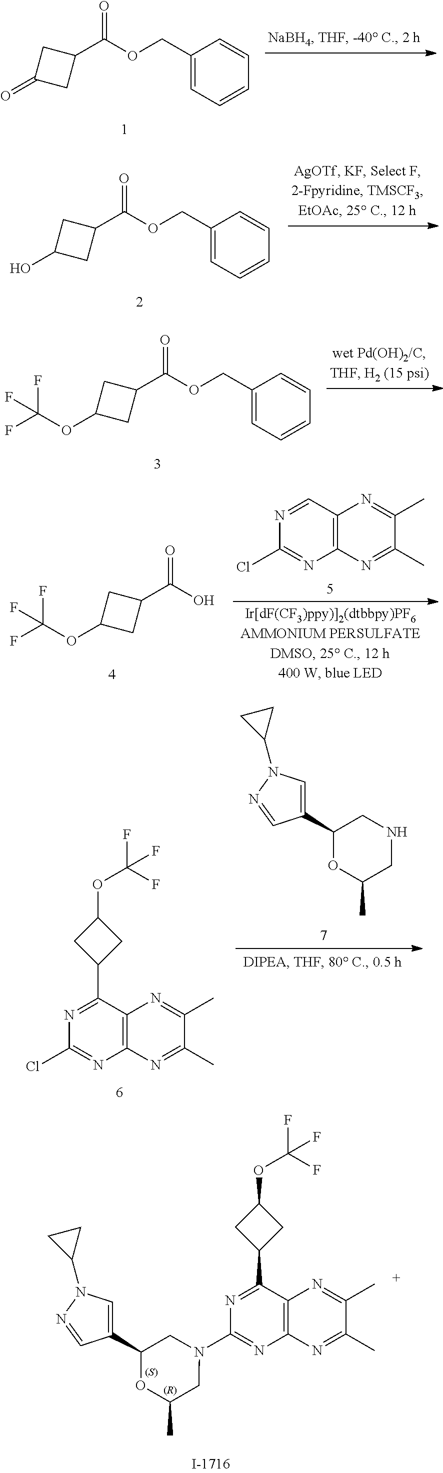

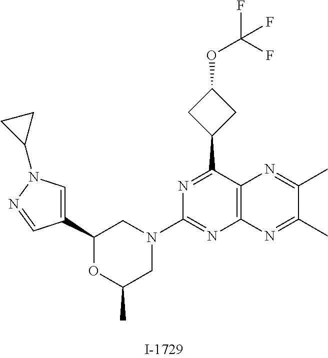 [SMILES: Cc1nc2nc(N3C[C@@H](C)O[C@@H](c4cnn(C5CC5)c4)C3)nc([C@H]3C[C@H](OC(F)(F)F)C3)c2nc1C]

Step 1: To a solution of benzyl 3-oxocyclobutanecarboxylate (1.00 eq, 1.00 g, 4.90 mmol) in methanol (10 mL) was added NaBH₄ (1.00 eq, 185 mg, 4.90 mmol) at −40° C. then the reaction mixture was stirred for 1 h. TLC (PE:EtOAc=3:1, Rf=0.1) showed starting material consumed and desired product plot appeared. The reaction mixture was quenched with aq. NH₄Cl (20 mL) under 0° C. slowly. Then the mixture was diluted with water (10 mL), extracted with EtOAc (20 mL three times). The combined organic phase was washed by brine (20 mL), dried by Na₂SO₄ and purified by reverse flash (0.1% v/v FA condition, 30% to 60%) and lyophilized to give benzyl 3-hydroxycyclobutanecarboxylate (1.00 g, 4.85 mmol, 99% yield) as a light-yellow oil. ¹H NMR (400 MHz, chloroform-d) 6=7.42-7.30 (m, 5H), 5.13 (s, 2H), 4.25-4.17 (m, 1H), 2.72-2.65 (m, 1H), 2.65-2.56 (m, 2H), 2.23-2.15 (m, 2H).

Step 2: Silver trifluoromethanesulfonate (4.00 eq, 3987 mg, 15.5 mmol), 1-chloromethyl-4-fluoro-1,4-diazoniabicyclo[2.2.2]octane bis(tetrafluoroborate) (1.50 eq, 2061 mg, 5.82 mmol) and KF (4.00 eq, 898 mg, 15.5 mmol) was added to a solution of benzyl 3-hydroxycyclobutanecarboxylate (1.00 eq, 800 mg, 3.88 mmol) in Ethyl acetate (10 mL), followed by 2-fluoropyridine (4.00 eq, 1.3 mL, 15.5 mmol) and (trifluoromethyl)trimethylsilane (2.50 eq, 1.5 mL, 9.70 mmol) was added dropwise to the mixture then the mixture was stirred at 25° C. for 36 h. The reaction mixture was filtered and washed the cake with EtOAc (50 mL). The combined filtrate was purified by reversed phase column (0.1% v/v FA condition, 30% to 60%) and lyophilized to give benzyl 3-(trifluoromethoxy)cyclobutanecarboxylate (790 mg, 2.88 mmol, 74% yield) as yellow oil, HPLC purity=56% (220 nm); Retention time=0.680 min. ¹H NMR (400 MHz, chloroform-d) 6=7.42-7.32 (m, 5H), 5.15 (s, 2H), 4.58 (quin, J=7.5 Hz, 1H), 2.83-2.72 (m, 1H), 2.65 (dtd, J=2.6, 7.2, 9.7 Hz, 2H), 2.60-2.48 (m, 2H). ¹⁹F NMR (377 MHz, chloroform-d) 6=−59.56 (s, 1F).

Step 3: To a solution of benzyl 3-(trifluoromethoxy)cyclobutanecarboxylate (1.00 eq, 864 mg, 3.15 mmol) in THF (10 mL) was added wet Pd(OH)₂ (0.119 eq, 263 mg, 0.375 mmol) under N₂ atmosphere, the mixture was purged by H₂ for 3 times, then the mixture was stirred at 25° C. for 12 h under H₂ atmosphere (balloon, 15 psi). The reaction mixture was filtered directly and washed the cake with EtOAc (20 mL). The combined filtrate was concentrated to give crude 3-(trifluoromethoxy)cyclobutanecarboxylic acid (525 mg, 2.85 mmol, 90% yield) as dark green oil. ¹H NMR (400 MHz, chloroform-d) 6=4.69-4.50 (m, 1H), 2.84-2.73 (m, 1H), 2.73-2.64 (m, 2H), 2.62-2.50 (m, 2H). ¹⁹F NMR (376 MHz, chloroform-d) δ=−59.61 (s, 1F).

Step 4: A reaction vial under argon was charged with Ir[dF(CF₃)ppy)]₂ (dtbbpy)PF₆ (0.00500 eq, 4.3 mg, 0.00385 mmol), ammonium persulfate (4.00 eq, 704 mg, 3.08 mmol), 3-(trifluoromethoxy)cyclobutanecarboxylic acid (3.00 eq, 426 mg, 2.31 mmol) and 2-chloro-6,7-dimethyl-pteridine (1.00 eq, 150 mg, 0.771 mmol). Anhydrous DMSO (5 mL) was added, the vial was sealed and placed under nitrogen. The reaction was stirred and irradiated with a 34 W blue LED lamp (7 cm away), with cooling fan to keep the reaction temperature at 25° C. for 14 hr. LCMS showed starting material was consumed and desired product (38%, Rt: 0.958 min; [M+H]⁺=333.0 at 220 nm) was formed. The reaction mixture was poured into water (20 mL), extracted with EtOAc (20 mL for three times). The combined organic phase was washed with brine (20 mL), dried over Na₂SO₄, purified by flash column (PE to EtOAc condition, 40% to 70%, Rf=0.5 under PE:EtOAc=1:3) and concentrated to give 2-chloro-6,7-dimethyl-4-[3-(trifluoromethoxy)cyclobutyl]pteridine (103 mg, 0.310 mmol, 40.17% yield) as brown solid. LCMS: [M+H]⁺=475.1; Retention time=0.644 min. ¹H NMR (400 MHz, chloroform-d) 6=4.84 (quin, J=7.6 Hz, 1H), 4.43-4.31 (m, 1H), 2.95-2.84 (m, 4H), 2.83 (s, 3H), 2.78 (s, 3H). ¹⁹F NMR (376 MHz, chloroform-d) 6=−59.42 (s, 1F).

Step 5: To a solution of (2S,6R)-2-(1-cyclopropylpyrazol-4-yl)-6-methyl-morpholine (2.00 eq, 72 mg, 0.349 mmol), 2-chloro-6,7-dimethyl-4-[3-(trifluoromethoxy)cyclobutyl]pteridine (1.00 eq, 58 mg, 0.174 mmol) in THF (1 mL) was added DIEA (4.00 eq, 0.12 mL, 0.697 mmol), then the mixture was stirred at 80° C. for 20 min. LCMS showed starting material consumed and desired product (68%, Rt: 0.671 min; [M+H]⁺=504.4 at 220 nm) was formed. The reaction mixture was diluted with water (10 mL) and EtOAc (10 mL), then the mixture was filtered and washed the cake with EtOAc (10 mL five times). The filtrate was extracted with EtOAc (10 mL three times). The combined organic layers were washed with brine (10 mL), dried over Na₂SO₄ and purified by flash column (Column: Phenomenex Synergi C18 150*25 mm, 10 um; Condition: water (FA)-ACN) to give (2S,6R)-2-(1-cyclopropylpyrazol-4-yl)-4-[6,7-dimethyl-4-[3-(trifluoromethoxy)cyclobutyl]pteridin-2-yl]-6-methyl-morpholine (20 mg, 0.0393 mmol, 22.56% yield) as yellow solid and (2S,6R)-2-(1-cyclopropylpyrazol-4-yl)-4-[6,7-dimethyl-4-[3-(trifluoromethoxy)cyclobutyl]pteridin-2-yl]-6-methyl-morpholine (13 mg, 0.0260 mmol, 14.92% yield) as yellow solid. LCMS: [M+H]⁺=504.2, purity=100% (220 nm); Retention time=1.007 min. ¹H NMR (400 MHz, chloroform-d) 6=7.60-7.54 (m, 2H), 5.16-5.08 (m, 1H), 5.06-4.96 (m, 1H), 4.82 (quin, J=7.6 Hz, 1H), 4.61 (dd, J=2.5, 10.9 Hz, 1H), 4.29-4.16 (m, 1H), 3.90-3.77 (m, 1H), 3.60 (tt, J=3.7, 7.2 Hz, 1H), 3.16-3.02 (m, 1H), 2.91-2.80 (m, 3H), 2.76-2.67 (m, 5H), 2.63 (s, 3H), 1.35 (br d, J=5.8 Hz, 3H), 1.14 (br s, 2H), 1.08-0.99 (m, 2H). ¹⁹F NMR (376 MHz, chloroform-d) δ=−59.21 (s, 1F). A second peak [M+H]⁺=504.2, purity=98% (220 nm); Retention time=1.017 min. ¹H NMR (400 MHz, chloroform-d) 6=7.55 (br d, J=6.5 Hz, 2H), 5.18-4.91 (m, 3H), 4.80-4.68 (m, 1H), 4.67-4.58 (m, 1H), 3.91-3.78 (m, 1H), 3.65-3.54 (m, 1H), 3.16-3.02 (m, 1H), 2.91-2.67 (m, 8H), 2.66-2.57 (m, 3H), 1.35 (br d, J=6.1 Hz, 3H), 1.18-1.10 (m, 2H), 1.07-0.97 (m, 2H). ¹⁹F NMR (376 MHz, chloroform-d) δ=−59.17 (s, 1F).

### Synthesis of Compound I-1733

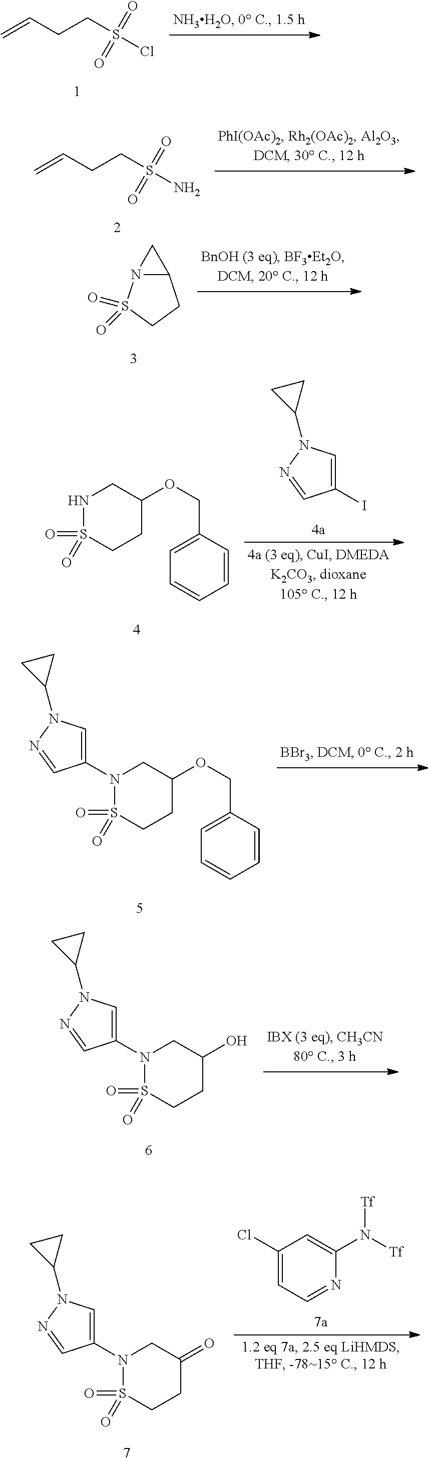

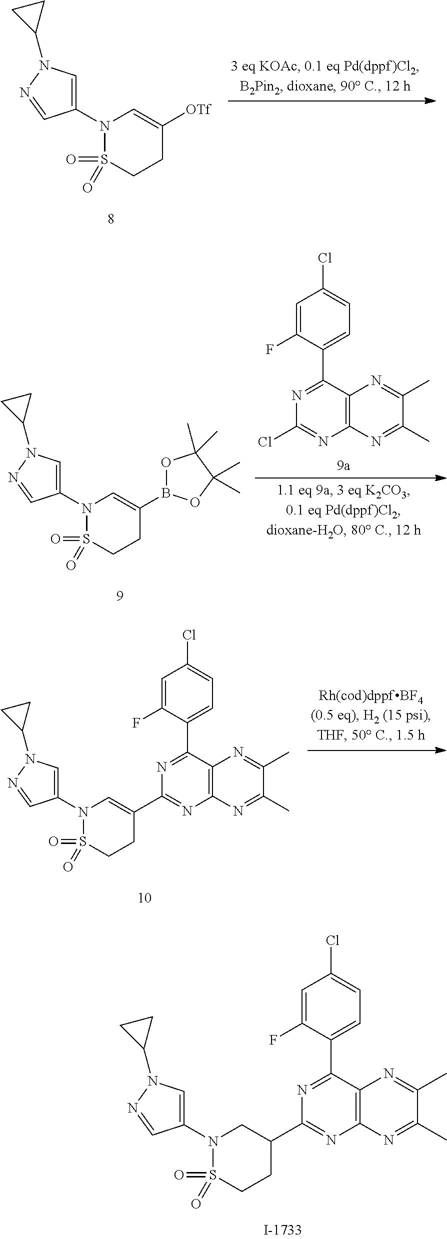

Step 1: To a solution of but-3-ene-1-sulfonyl chloride (1.00 eq, 4300 mg, 27.8 mmol) in MeCN (16 mL) was added NH₃·H₂O (43.1 eq, 40 mL, 1200 mmol) in MeCN (8 mL) dropwise at 0° C., then the mixture was stirred at 0° C. for 2 h. Water (80 mL) was added to the mixture, then the reaction mixture was extracted with DCM (150 mL*3), then the water phase was extracted with EtOAc (150 mL* 3), combined the organic phase, dried over Na₂SO₄, filtered and concentrated under reduced pressure to give but-3-ene-1-sulfonamide (3200 mg, 23.7 mmol, 85.12% yield) as white solid. ¹H NMR (400 MHz, CDCl₃, 298 K) Shift (ppm)=5.86 (tdd, J=6.6, 10.3, 17.0 Hz, 1H), 5.26-5.10 (m, 2H), 4.58 (br s, 2H), 3.30-3.18 (m, 2H), 2.71-2.58 (m, 2H).

Step 2: To a solution of but-3-ene-1-sulfonamide (1.00 eq, 3200 mg, 23.7 mmol) in DCM (100 mL) were added PhI(OAc)₂ (1.50 eq, 11437 mg, 35.5 mmol), Rh₂ (OAc)₄ (0.0500 eq, 528 mg, 1.18 mmol) and Al₂O₃ (2.50 eq, 6034 mg, 59.2 mmol) in one portion, then the mixture was stirred at 30° C. for 12 h under N₂. TLC showed starting material was consumed, desired peak was formed. (PE/EtOAc=1/2, the desired product Rf=0.3, starting material Rf=0.6, phosphomolybdic acid as chromogenic agent). The mixture was evaporated to give the residue, the residue was purified by column chromatography (hexane/ethyl acetate=0-70%, PE/EtOAc=1/2, the desired product Rf=0.3) to obtain 2D6-thia-1-azabicyclo[3.1.0]hexane 2,2-dioxide (2800 mg, 21.0 mmol, 88.82% yield) as white solid. ¹H NMR (400 MHz, CDCl₃, 298 K) Shift (ppm)=3.23-3.16 (m, 1H), 3.15-3.06 (m, 1H), 2.87-2.74 (m, 1H), 2.70-2.62 (m, 2H), 2.47 (dd, J=2.7, 5.1 Hz, 1H), 2.30 (dd, J=2.8, 4.3 Hz, 1H).

Step 3: To a solution of BnOH (3.00 eq, 5.6 mL, 54.1 mmol) and 2D6-thia-1-azabicyclo[3.1.0]hexane 2,2-dioxide (1.00 eq, 2400 mg, 18.0 mmol) in DCM (40 mL) was added BF₃.Et₂O (0.1000 eq, 256 mg, 1.80 mmol) in portions at 15° C., then the mixture was stirred at 15° C. under N₂ for 3 h. LCMS showed raw material was consumed completely and the major peak showed MS (88%, Rt: 0.734 min; [M+H]⁺=242.1 at 220 nm). TLC found desired product (hexane/ethyl acetate=1:1, Rf=0.6). The mixture was evaporated to give the residue, the residue was purified by column chromatography (hexane/ethyl acetate=0-60%, PE/EtOAc=1/1, the desired product Rf=0.6) to obtain 4-benzyloxythiazinane 1,1-dioxide (3200 mg, 12.6 mmol, 70% yield) as colorless oil. LCMS: 97.5% purity, Rt: 0.658 min; [M+H]⁺=242.1 at 220 nm); ¹H NMR (400 MHz, CDCl₃, 298 K) Shift (ppm)=7.44-7.30 (m, 5H), 4.65-4.52 (m, 2H), 4.47 (br d, J=8.0 Hz, 1H), 3.63-3.45 (m, 3H), 3.44-3.31 (m, 1H), 3.10 (td, J=3.9, 13.5 Hz, 1H), 2.46-2.38 (m, 1H), 2.36-2.27 (m, 1H).

Step 4: To a solution of 4-benzyloxythiazinane 1,1-dioxide (1.00 eq, 2900 mg, 12.0 mmol) and 1-cyclopropyl-4-iodo-pyrazole (2.00 eq, 5625 mg, 24.0 mmol) in 1,4-Dioxane (40 mL) were added CuI (3.00 eq, 6866 mg, 36.1 mmol), K₂CO₃ (3.00 eq, 4983 mg, 36.1 mmol) and DMEDA (4.00 eq, 4237 mg, 48.1 mmol) in one portion at 15° C., then the mixture was stirred at 105° C. for 12 h. LCMS showed raw material was consumed completely and the major peak showed MS (40.7%, Rt: 0.766 min; [M+H]⁺=348.2 at 220 nm). The reaction mixture was filtered and the filter cake was washed with DCM (100 mL), combined the solvent, evaporate to give the residue, the residue was purified by column chromatography (hexane/ethyl acetate=0-60%, PE/EtOAc=1/1, the desired product Rf=0.4) to obtain 4-benzyloxy-2-(1-cyclopropylpyrazol-4-yl)thiazinane 1,1-dioxide (4000 mg, 10.9 mmol, 91.01% yield) as colorless oil, 41% purity, Rt: 0.766 min; [M+H]⁺=348.2 at 220 nm); ¹H NMR (400 MHz, CDCl₃, 298 K) Shift (ppm)=7.50 (s, 1H), 7.46 (s, 1H), 7.41-7.31 (m, 5H), 4.58 (s, 2H), 3.94-3.87 (m, 1H), 3.74-3.66 (m, 2H), 3.51 (tt, J=3.8, 7.3 Hz, 1H), 3.42 (td, J=7.5, 13.4 Hz, 1H), 3.08 (td, J=4.9, 13.3 Hz, 1H), 2.48 (td, J=4.1, 7.7 Hz, 2H), 1.08-1.02 (m, 2H), 1.00-0.93 (m, 2H).

Step 5: To a solution of 4-benzyloxy-2-(1-cyclopropylpyrazol-4-yl)thiazinane 1,1-dioxide (1.00 eq, 1200 mg, 3.45 mmol) in DCM (30 mL) was added BBr₃ (3.00 eq, 3.3 mL, 10.4 mmol) in DCM (3 mL) dropwise at 0° C., then the mixture was stirred at 0° C. under N₂ for 4 h. TLC showed starting material was consumed, new peak was formed. (DCM/MeOH=15/1, the desired product Rf=0.3, phosphomolybdic acid as chromogenic agent). The reaction mixture was quenched with NaHCO₃ solution (10 mL), then the mixture was evaporated to give the residue, the residue was purified by column chromatography (hexane/ethyl acetate=10:1˜0:1 then ethyl acetate: MeOH=3:1, the desired product Rf=0.3, DCM/MeOH=15/1) to obtain 2-(1-cyclopropylpyrazol-4-yl)-1,1-dioxo-thiazinan-4-ol (500 mg, 1.94 mmol, 56.26% yield) as yellow oil. ¹H NMR (400 MHz, CDCl3, 296 K) Shift (ppm)=7.51 (br s, 1H), 7.43 (br s, 1H), 3.91 (br s, 1H), 3.72 (br d, J=13.6 Hz, 1H), 3.54-3.41 (m, 2H), 3.30 (br dd, J=3.9, 13.2 Hz, 2H), 3.00-2.91 (m, 1H), 2.38-2.27 (m, 1H), 2.26-2.16 (m, 1H), 1.00 (br s, 2H), 0.90 (br d, J=5.0 Hz, 2H).

Step 6: To a solution of 2-(1-cyclopropylpyrazol-4-yl)-1,1-dioxo-thiazinan-4-ol (1.00 eq, 1100 mg, 4.27 mmol) in MeCN (25 mL) was added IBX (3.00 eq, 3386 mg, 12.8 mmol) in one portion, then the mixture was stirred at 90° C. for 3 h. The mixture was filtered and then the solvent was evaporated to give the residue. The residue was purified by reversed-phase column (C18 150*40 mm* 15 um, 0.1% FA) and the solvent was lyophilized to give 2-(1-cyclopropylpyrazol-4-yl)-1,1-dioxo-thiazinan-4-one (800 mg, 3.13 mmol, 73.30% yield) as yellow oil, 83% purity, Rt: 0.209 min; [M+H]⁺=256.0 and [M+H+18]⁺=274.2 at 220 nm; ¹H NMR (400 MHz, CDCl3, 298 K) Shift (ppm)=7.50 (s, 1H), 7.39 (s, 1H), 4.30 (s, 2H), 3.57 (td, J=3.6, 7.3 Hz, 1H), 3.51 (t, J=6.8 Hz, 2H), 3.10 (t, J=6.8 Hz, 2H), 1.16-1.10 (m, 2H), 1.07-1.01 (m, 2H).

Step 7: To a colorless mixture of 2-(1-cyclopropylpyrazol-4-yl)-1,1-dioxo-thiazinan-4-one (1.00 eq, 400 mg, 1.57 mmol) in THF (30 mL) was added LiHMDS (1.50 eq, 2.4 mL, 2.35 mmol) dropwise at −78° C. under N₂ atmosphere, then the mixture was stirred at −78° C. for 1 hour, N-(4-chloro-2-pyridyl)-1,1,1-trifluoro-N-(trifluoromethylsulfonyl)methanesulfonamide (1.00 eq, 615 mg, 1.57 mmol) in THF (6 mL) was added dropwise at −78° C. under N₂ atmosphere, then the mixture was stirred at −78° C. for 0.5 hour and at 15° C. for 12 hours to give a red solution. LCMS showed raw material was consumed completely and showed desired MS (26%, Rt: 0.860 min; [M+H]⁺=387.9 at 220 nm, ESI⁺). NH₄Cl (2 mL) was added to the mixture, then NaHCO₃ solution (3 mL) was added, the mixture was purified by purified by reversed-phase column (C18 150*40 mm*15 um, 0.1% FA) and the solvent was lyophilized to give [2-(1-cyclopropylpyrazol-4-yl)-1,1-dioxo-5,6-dihydrothiazin-4-yl]trifluoromethanesulfonate (220 mg, 0.568 mmol, 36.25% yield) as yellow oil, 85% purity, Rt: 0.787 min; [M+H]⁺=388.1 at 220 nm).

Step 8: To a colorless mixture of [2-(1-cyclopropylpyrazol-4-yl)-1,1-dioxo-5,6-dihydrothiazin-4-yl] trifluoromethanesulfonate (1.00 eq, 420 mg, 1.08 mmol) in 1,4-Dioxane (12 mL) was added B₂pin₂ (1.50 eq, 413 mg, 1.63 mmol), KOAc (3.00 eq, 319 mg, 3.25 mmol), Pd(dppf)Cl₂ DCM (0.100 eq, 88 mg, 0.108 mmol) in one portion, then the mixture was stirred at 90° C. for 12 hr under N₂ atmosphere to give a black solution. LCMS showed raw material was consumed completely and showed desired MS (44%, Rt: 0.895 min; [M+H]⁺=366.0 at 220 nm, ESI⁺). The reaction mixture was evaporated to give the residue, the residue was purified by reversed-phase column (C18 150*40 mm* 15 um, 0.1% FA) and the solvent was lyophilized to give 2-(1-cyclopropylpyrazol-4-yl)-4-(4,4,5,5-tetramethyl-1,3,2-dioxaborolan-2-yl)-5,6-dihydrothiazine 1,1-dioxide (270 mg, 0.665 mmol, 61.36% yield) as yellow solid, 74% purity, Rt: 0.89 min; [M+H]⁺=366.0 at 220 nm).

Step 9: To a mixture of 2-chloro-4-(4-chloro-2-fluoro-phenyl)-6,7-dimethyl-pteridine (1.20 eq, 127 mg, 0.394 mmol) in 1,4-dioxane (12 mL) was added 2-(1-cyclopropylpyrazol-4-yl)-4-(4,4,5,5-tetramethyl-1,3,2-dioxaborolan-2-yl)-5,6-dihydrothiazine 1,1-dioxide (1.00 eq, 120 mg, 0.329 mmol), K₂CO₃ (3.00 eq, 136 mg, 0.986 mmol), Pd(dppf)Cl₂ DCM (0.100 eq, 27 mg, 0.0329 mmol), then the brown mixture was stirred at 80° C. for 12 hrs under N₂ atmosphere to give a black solution. LCMS: showed raw material was consumed completely and showed desired MS (60%, Rt: 0.979 min; [M+H]⁺=526.0 at 220 nm, ESI⁺). The reaction mixture was filtered and the filter cake was washed with DCM (30 mL), combined the solvent, evaporate to give the residue, the residue was purified by reversed-phase column (C18 150*40 mm* 15 um, 0.1% FA) and the solvent was lyophilized to give 4-[4-(4-chloro-2-fluoro-phenyl)-6,7-dimethyl-pteridin-2-yl]-2-(1-cyclopropylpyrazol-4-yl)-5,6-dihydrothiazine 1,1-dioxide (80 mg, 0.137 mmol, 41.67% yield) as yellow solid, 94% purity, Rt: 0.96 min; [M+H]⁺=366.0 at 220 nm).

Step 10: A solution of 4-[4-(4-chloro-2-fluoro-phenyl)-6,7-dimethyl-pteridin-2-yl]-2-(1-cyclopropylpyrazol-4-yl)-5,6-dihydrothiazine 1,1-dioxide (1.00 eq, 120 mg, 0.228 mmol) in THF (25 mL) was added 1,1′-bis(di-i-propylphosphino)ferrocene(1,5-cyclooctadiene)rhodium(I) tetrafluoroborate (0.400 eq, 65 mg, 0.0913 mmol) under N₂ atmosphere, the mixture was purged by H₂ for 3 times, then stirred at 50° C. for 1.5 h under H₂ atmosphere (balloon, 15 psi). LCMS found desired MS (528.2, [M+H]⁺, ESI⁺). The reaction mixture was filtered and then the filtrate was evaporated to give the residue. The residue was purified by Prep-TLC (DCM/MeOH=20/1, the desired product Rf=0.4) to give −20 mg crude product (˜90% purity) and 4-[4-(4-chloro-2-fluoro-phenyl)-6,7-dimethyl-pteridin-2-yl]-2-(1-cyclopropylpyrazol-4-yl)thiazinane 1,1-dioxide (52 mg, 0.0962 mmol, 42.18% yield) as yellow solid. The pure compound was lyophilized to yellow solid, racemate, 97.7% purity, Rt: 0.925 min; [M+H]⁺=528.1 at 220 nm; HPLC: Rt: 2.16 min, 97.2% purity at 215 nm. ¹H NMR (400 MHz, CDCl3, 298 K) Shift (ppm)=7.67 (t, J=7.8 Hz, 1H), 7.56 (s, 1H), 7.47 (s, 1H), 7.36 (dd, J=1.7, 8.3 Hz, 1H), 7.30 (dd, J=1.8, 9.5 Hz, 1H), 4.49 (dd, J=9.5, 13.4 Hz, 1H), 4.18-4.10 (m, 1H), 3.82-3.74 (m, 1H), 3.59-3.45 (m, 2H), 3.37-3.28 (m, 1H), 3.04 (dtd, J=3.7, 10.6, 14.2 Hz, 1H), 2.89-2.83 (m, 4H), 2.76 (s, 3H), 1.15-1.08 (m, 2H), 1.03-0.96 (m, 2H). ¹⁹F NMR (376.5 MHz, CDCl₃, 298 K), Shift (ppm)=−108.40. Chiral SFC showed the product of the reaction was a racemic mixture with a 1:1 ratio of two stereoisomers.

### Synthesis of Compounds I-1736 and I-1738

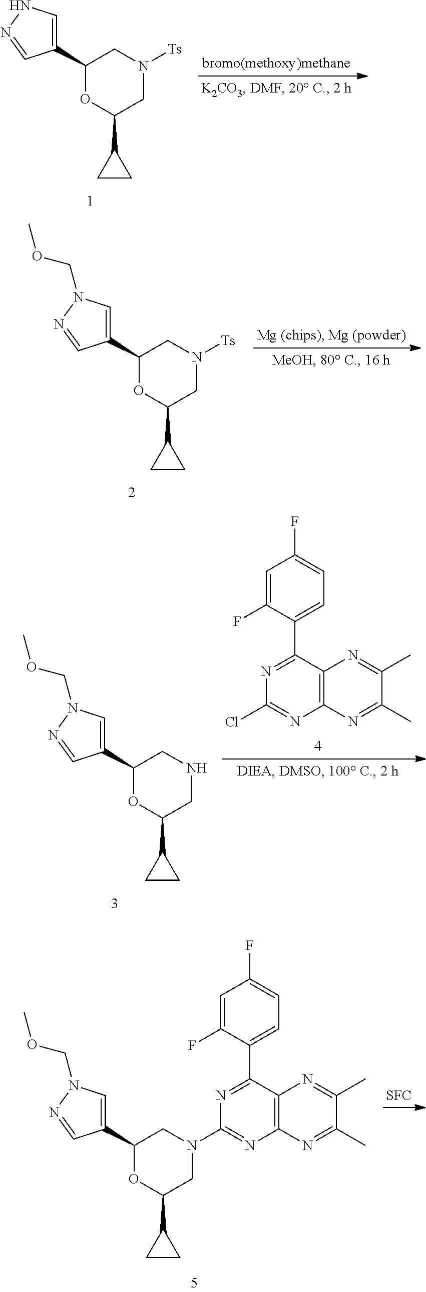

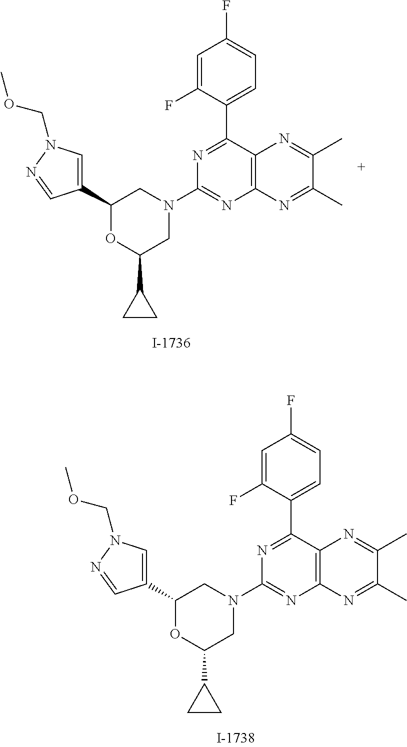

Step 1: To a solution of (2R,6S)-2-cyclopropyl-4-(p-tolylsulfonyl)-6-(1H-pyrazol-4-yl) morpholine (1.00 eq, 600 mg, 1.73 mmol) and K₂CO₃ (2.00 eq, 477 mg, 3.45 mmol) in DMF (7 mL) was added bromo (methoxy) methane (1.70 eq, 0.24 mL, 2.94 mmol). The mixture was stirred at 25° C. for 2 hours. LCMS showed the starting material was nearly consumed completely and a major peak with desired product mass (69%, MS: 392.2 [M+H]⁺, ESI pos). The mixture was extracted with EtOAc (500 mL). The combined organic layer was dried over anhydrous sodium sulphate, concentrated under reduced pressure to get the crude product. The crude product was purified by column chromatography on silica gel eluted with petroleum ether/ethyl acetate=100: 1 to 1: 1, R_{f}=0.4 to afford (2R,6S)-2-cyclopropyl-6-[1-(methoxymethyl) pyrazol-4-yl]-4-(p-tolylsulfonyl) morpholine (600 mg, 1.53 mmol, 88.75% yield) as yellow oil. LCMS: (M+H)⁺=392.2; purity=97% (220 nm); retention time=0.733 min. ¹H NMR (400 MHz, chloroform-d) δ ppm 0.26-0.36 (m, 1H) 0.38-0.46 (m, 1H) 0.49-0.62 (m, 1H) 0.74-0.86 (m, 1H) 2.16-2.29 (m, 1H) 2.46 (s, 2H) 2.89 (s, 3H) 2.96 (s, 3H) 3.02 (ddd, J=10.38, 8.19, 2.44 Hz, 1H) 3.33 (s, 2H) 3.68-3.79 (m, 1H) 4.56-4.66 (m, 1H) 5.33 (s, 1H) 7.36 (d, J=8.00 Hz, 1H) 7.50 (d, J=11.38 Hz, 1H) 7.65 (d, J=8.25 Hz, 1H) 8.02 (s, 1H).

Step 2: To a solution of (2R,6S)-2-cyclopropyl-6-[1-(methoxymethyl)pyrazol-4-yl]-4-(p-tolylsulfonyl) morpholine (1.00 eq, 600 mg, 1.53 mmol) in Methanol (15 mL) was added Mg (chips) (10.0 eq, 368 mg, 15.3 mmol) and Mg (powder) (10.0 eq, 368 mg, 15.3 mmol). The mixture was stirred at 80° C. for 12 hours under N₂ atmosphere. LCMS showed the starting material was still remained. Then the reaction mixture was added Mg (chips) (10.0 eq, 368 mg, 15.3 mmol). The mixture was stirred at 80° C. for another 12 hours under N₂ atmosphere. LCMS showed the starting material was consumed completely. The reaction mixture was filtered by celite to afford crude product, the crude product was used directly for the next step. LCMS: (M+H)⁺=238.1; purity=57% (220 nm); Retention time=0.552 min.

Step 3: To a solution of 2-chloro-4-(2, 4-difluorophenyl)-6, 7-dimethyl-pteridine (1.00 eq, 200 mg, 0.652 mmol) and (2R,6S)-2-cyclopropyl-6-[1-(methoxymethyl) pyrazol-4-yl] morpholine (2.00 eq, 309 mg, 1.30 mmol) in DMSO (4 mL) was added DIEA (5.00 eq, 0.54 mL, 3.26 mmol). The mixture was stirred at 100° C. for 2 hours. LCMS showed the starting material was consumed completely with the appearance of a major peak with the desired product mass (51%, MS: 508.3 [M+H]⁺, ESI pos). The mixture was concentrated under reduced pressure and purified by reversed-phase chromatography (0.5% FA) to give 200 mg yellow solid, checked by LCMS, (M+H)⁺=508.3; purity=100% (220 nm); retention time=0.909 min.

Step 4: The mixture from step 3 was purified by SFC (DAICEL CHIRALPAK AD (250 mm*30 mm, 10 um), Mobile phase: Phase A for CO₂, and Phase B for MeOH (0.05% DEA); Gradient elution: 40% MeOH (0.05% DEA) in CO₂, Flow rate: 3 mL/min; Detector: PDA, Column Temp: 35C; Back Pressure: 100 Bar) to give (2R,6S)-2-cyclopropyl-4-[4-(2, 4-difluorophenyl)-6,7-dimethyl-pteridin-2-yl]-6-[1-(methoxymethyl) pyrazol-4-yl]morpholine (79 mg, 0.155 mmol, 23.84% yield) as yellow solid and the (2S,6R)-2-cyclopropyl-4-[4-(2,4-difluorophenyl)-6,7-dimethyl-pteridin-2-yl]-6-[1-(methoxymethyl)pyrazol-4-yl] morpholine (74 mg, 0.145 mmol, 22.18% yield) as yellow solid. LCMS: (M+H)⁺=508.2; purity=100% (220 nm); Retention time=0.970 min. ¹H NMR (400 MHz, chloroform-d) δ ppm 0.35-0.53 (m, 2H) 0.55-0.67 (m, 2H) 0.96-1.08 (m, 1H) 2.60 (s, 3H) 2.72 (s, 3H) 3.01-3.15 (m, 3H) 3.36 (s, 3H) 4.58 (dd, J=10.88, 2.45 Hz, 1H) 4.98-5.16 (m, 2H) 5.38 (s, 2H) 6.94-7.02 (m, 1H) 7.02-7.10 (m, 1H) 7.66 (d, J=5.50 Hz, 2H) 7.69-7.77 (m, 1H), and LCMS: (M+H)⁺=508.2; purity=100% (220 nm); Retention time=0.969 min. HPLC: Retention time=2.397 min, 99% purity at 220 nm. ¹H NMR (400 MHz, chloroform-d) δ ppm 0.33 (s, 2H) 0.55-0.71 (m, 2H) 0.95-1.09 (m, 1H) 2.60 (s, 3H) 2.72 (s, 3H) 2.99-3.14 (m, 3H) 3.36 (s, 3H) 4.59 (dd, J=10.76, 2.45 Hz, 1H) 5.13 (br s, 2H) 5.38 (s, 2H) 6.95-7.02 (m, 1H) 7.02-7.10 (m, 1H) 7.66 (d, J=5.50 Hz, 2H) 7.69-7.77 (m, 1H).

### Example A3: In Vitro Assay Data

### In Vitro Measurement of Triggering Receptor Expressed on Myeloid Cells 2 Activity Using Cellular Phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 μL of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluourescence intensity was measured using a Perkin Elmer Envision plate reader. Intensities were used to generate a standard curve, and % activation was calculated. Curve fitting was performed using Prism v9 software, log(agonist) vs response-variable slope (four parameters), and EC50s were calculated from the curve fit.

The results presented in Table D have been generated with the in vitro assay described above. This assay may be used to test any of the compounds described herein to assess and characterize a compound's ability to act as an agonist of TREM2.

Compounds designated as “A” demonstrated an EC50 of ≤0.05 μM. Compounds designated as “B” demonstrated an EC50 >0.05 μM and ≤0.5 μM. Compounds designated as “C” demonstrated an EC50 >0.5 μM and ≤3.0 μM. Compounds designated as “D” demonstrated an EC50 >3.0 μM and ≤100 μM. Compounds designated as “-” had not been tested as of the filing of the present application, but can be tested using the methods described herein.

TABLE D
hTREM2 EC50 Data (HEK293 Cells)

| Ex # | hTREM2 EC50 μM |
|---|---|
| 1 | B |
| 2 | B |
| 3 | B |
| 4 | B |
| 5 | B |
| 6 | B |
| 7 | A |
| 8 | A |
| 9 | A |
| 10 | A |
| 11 | B |
| 12 | A |
| 13 | A |
| 14 | A |
| 15 | — |
| 16 | B |
| 17 | A |
| 18 | A |
| 19 | B |
| 20 | A |
| 21 | A |
| 22 | A |
| 23 | A |
| 24 | — |
| 25 | A |
| 26 | C |
| 27 | A |
| 28 | C |
| 29 | A |
| 30 | A |
| 31 | B |
| 32 | A |
| 33 | C |
| 34 | B |
| 35 | B |
| 36 | A |
| 37 | A |
| 38 | B |
| 39 | A |
| 40 | — |
| 41 | A |
| 42 | A |
| 43 | A |
| 44 | A |
| 45 | C |
| 46 | — |
| 47 | A |
| 48 | B |
| 49 | A |
| 50 | B |
| 51 | A |
| 52 | A |
| 53 | A |
| 54 | A |
| 55 | A |
| 56 | B |
| 57 | A |
| 58 | A |
| 59 | A |
| 60 | B |
| 61 | A |
| 62 | B |
| 63 | B |
| 64 | A |
| 65 | B |
| 66 | A |
| 67 | A |
| 68 | A |
| 69 | A |
| 70 | A |
| 71 | C |
| 72 | B |
| 73 | B |
| 74 | B |
| 75 | A |
| 76 | A |
| 77 | A |
| 78 | B |
| 79 | A |
| 80 | B |
| 81 | A |
| 82 | A |
| 83 | B |
| 84 | A |
| 85 | — |
| 86 | A |
| 87 | A |
| 88 | B |
| 89 | A |
| 90 | A |
| 91 | B |
| 92 | A |
| 93 | A |
| 94 | A |
| 95 | A |
| 96 | A |
| 97 | A |
| 98 | B |
| 99 | A |
| 100 | B |
| 101 | C |
| 102 | A |
| 103 | A |
| 104 | B |
| 105 | A |
| 106 | A |
| 107 | B |
| 108 | A |
| 109 | A |
| 110 | B |
| 111 | B |
| 112 | B |
| 113 | — |
| 114 | B |
| 115 | A |
| 116 | C |
| 117 | A |
| 118 | B |
| 119 | A |
| 120 | A |
| 121 | A |
| 122 | A |
| 123 | B |
| 124 | C |
| 125 | A |
| 126 | A |
| 127 | A |
| 128 | A |
| 129 | B |
| 130 | A |
| 131 | C |
| 132 | D |
| 133 | A |
| 134 | A |
| 135 | B |
| 136 | A |
| 137 | A |
| 138 | B |
| 139 | A |
| 140 | C |
| 141 | A |
| 142 | B |
| 143 | C |
| 144 | B |
| 145 | C |
| 146 | B |
| 147 | B |
| 148 | A |
| 149 | C |
| 150 | A |
| 151 | C |
| 152 | C |
| 153 | A |
| 154 | B |
| 155 | B |
| 156 | B |
| 157 | A |
| 158 | B |
| 159 | A |
| 160 | B |
| 161 | B |
| 162 | B |
| 163 | B |
| 164 | C |
| 165 | B |
| 166 | B |
| 167 | B |
| 168 | B |
| 169 | C |
| 170 | A |
| 171 | B |
| 172 | D |
| 173 | B |
| 174 | D |
| 175 | B |
| 176 | B |
| 177 | A |
| 178 | A |
| 179 | A |
| 180 | A |
| 181 | A |
| 182 | A |
| 183 | D |
| 184 | A |
| 185 | C |
| 186 | C |
| 187 | A |
| 188 | A |
| 189 | B |
| 190 | A |
| 191 | D |
| 192 | A |
| 193 | B |
| 194 | A |
| 195 | A |
| 196 | B |
| 197 | A |
| 198 | A |
| 199 | B |
| 200 | A |
| 201 | C |
| 202 | C |
| 203 | D |
| 204 | C |
| 205 | A |
| 206 | B |
| 207 | A |
| 208 | C |
| 209 | B |
| 210 | A |
| 211 | A |
| 212 | A |
| 213 | |
| 214 | B |
| 215 | B |
| 216 | A |
| 217 | A |
| 218 | A |
| 219 | A |
| 220 | C |
| 221 | A |
| 222 | C |
| 223 | B |
| 224 | A |
| 225 | C |
| 226 | C |
| 227 | C |
| 228 | B |
| 229 | B |
| 230 | D |
| 231 | B |
| 232 | A |
| 233 | A |
| 234 | A |
| 235 | A |
| 236 | B |
| 237 | A |
| 238 | B |
| 239 | B |
| 240 | B |
| 241 | B |
| 242 | A |
| 243 | B |
| 244 | B |
| 245 | C |
| 246 | D |
| 247 | A |
| 248 | |

TABLE D-2
hTREM2 EC50 Data (HEK293 Cells):

| I# | hTREM2 EC50 μM |
|---|---|
| 1071 | B |
| 1076 | A |
| 1081 | C |
| 1086 | C |
| 1091 | A |
| 1099 | A |
| 1104 | C |
| 1109 | B |
| 1114 | B |
| 1119 | B |
| 1129 | A |
| 1134 | B |
| 1139 | C |
| 1144 | C |
| 1150 | B |
| 1165 | D |
| 1170 | A |
| 1175 | C |
| 1177 | C |
| 1182 | C |
| 1187 | B |
| 1192 | C |
| 1197 | B |
| 1205 | A |
| 1210 | C |
| 1216 | A |
| 1221 | A |
| 1226 | A |
| 1231 | D |
| 1241 | A |
| 1260 | C |
| 1265 | B |
| 1270 | B |
| 1283 | A |
| 1288 | A |
| 1293 | A |
| 1312 | B |
| 1313 | B |
| 1318 | B |
| 1323 | A |
| 1328 | A |
| 1333 | B |
| 1334 | D |
| 1338 | B |
| 1365 | A |
| 1381 | A |
| 1391 | A |
| 1396 | D |
| 1401 | A |
| 1414 | A |
| 1432 | A |
| 1441 | A |
| 1446 | A |
| 1451 | C |
| 1456 | A |
| 1461 | B |
| 1462 | B |
| 1467 | A |
| 1472 | A |
| 1477 | B |
| 1482 | B |
| 1485 | A |
| 1490 | D |
| 1495 | B |
| 1500 | D |
| 1505 | C |
| 1510 | B |
| 1511 | B |
| 1513 | B |
| 1516 | C |
| 1521 | C |
| 1524 | A |
| 1529 | B |
| 1532 | B |
| 1538 | A |
| 1543 | A |
| 1552 | A |
| 1557 | B |
| 1562 | B |
| 1567 | D |
| 1572 | A |
| 1577 | A |
| 1582 | A |
| 1585 | A |
| 1588 | A |
| 1593 | A |
| 1598 | A |
| 1603 | C |
| 1609 | A |
| 1614 | A |
| 1619 | A |
| 1624 | C |
| 1633 | A |
| 1638 | A |
| 1643 | C |
| 1648 | B |
| 1653 | A |
| 1658 | B |
| 1663 | A |
| 1668 | A |
| 1673 | A |
| 1674 | B |
| 1739 | A |
| 1686 | A |
| 1691 | A |
| 1696 | C |
| 1698 | B |
| 1699 | B |
| 1700 | B |
| 1701 | A |
| 1702 | B |
| 1706 | A |
| 1744 | B |
| 1749 | A |
| 1711 | A |
| 1712 | B |
| 1729 | A |
| 1733 | B |
| 1736 | A |

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula IIIa 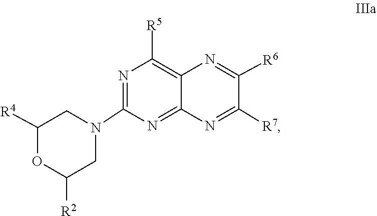
or a pharmaceutically acceptable salt thereof;
wherein
R² is H or methyl;
R⁴ is 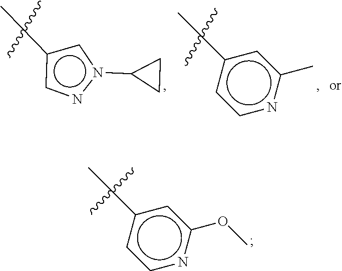
R⁵ is C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that:
when R⁴ is 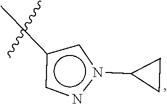
and R² is H, R⁵ is not 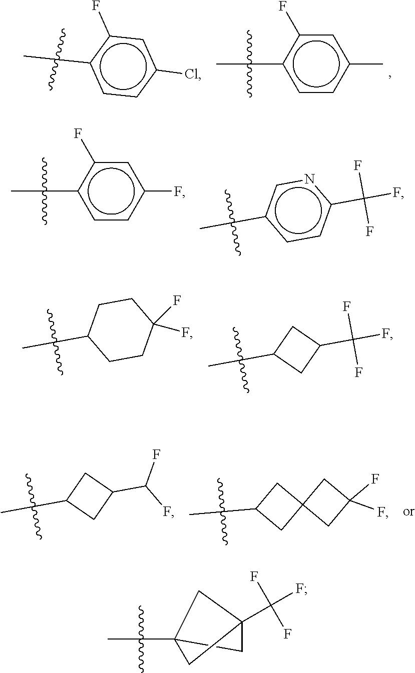
and
when R⁴ is 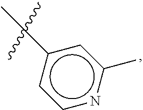
and R² is H, R⁵ is not

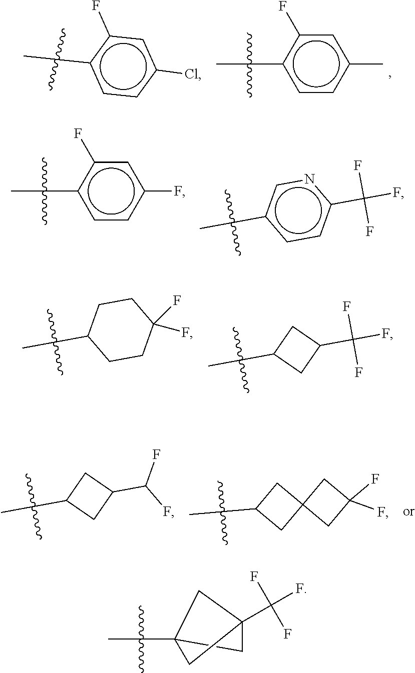

2. A compound of Formula IIIa 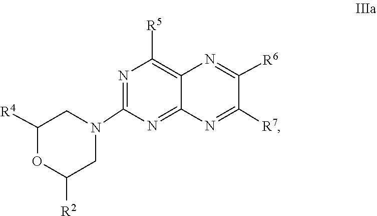
or a pharmaceutically acceptable salt thereof;
wherein
R² is H or methyl;
R⁴ is 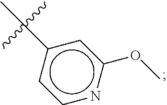
R⁵ is C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl.

3. A compound of Formula IIIa 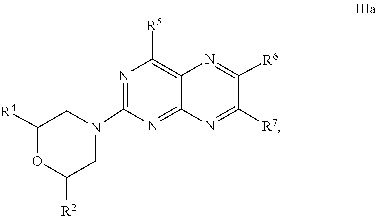
or a pharmaceutically acceptable salt thereof;
wherein
R² is methyl;
R⁴ is 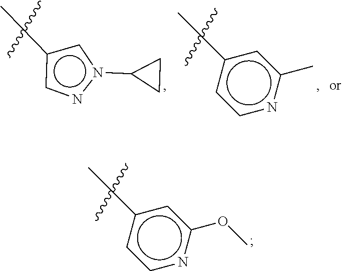
R⁵ is C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is Me.

4. A compound of Formula IIIa 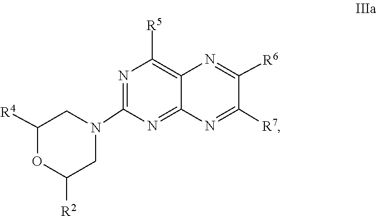
or a pharmaceutically acceptable salt thereof;
wherein
R² is H or methyl;
R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
R⁵ is 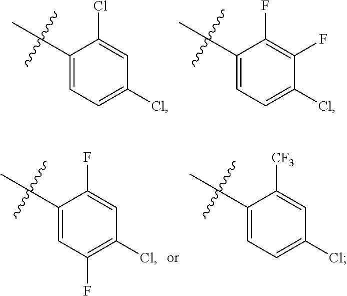 [SMILES: *c1ccc(Cl)cc1Cl ; *c1ccc(Cl)c(F)c1F ; *c1cc(F)c(Cl)cc1F ; *c1ccc(Cl)cc1C(F)(F)F]
R⁶ is H or methyl; and
R⁷ is Me.

5. A compound of Formula IIIb 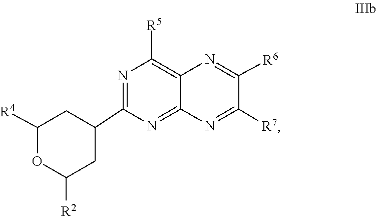
or a pharmaceutically acceptable salt thereof;
wherein
R² is H or methyl;
R⁴ is 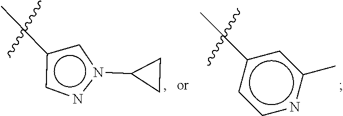
R⁵ is C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that:
when R⁴ is 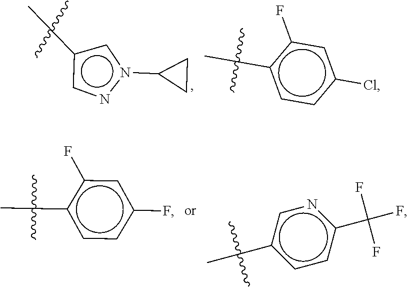
and
when R⁴ is 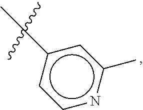
R⁵ is not

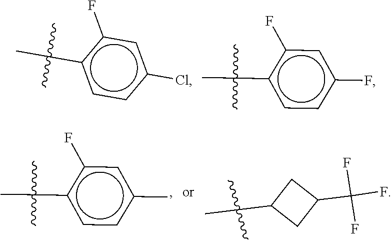

6. A compound of Formula IIIb 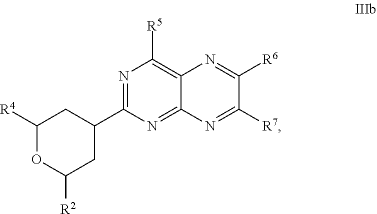
or a pharmaceutically acceptable salt thereof;
wherein
R² is H or methyl;
R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
R₅ is 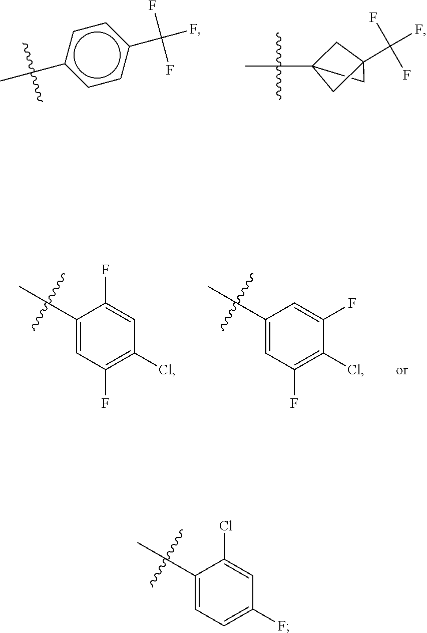
R⁶ is H or methyl; and
R⁷ is methyl;
provided that when R⁴ is 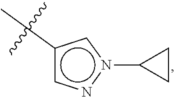
R⁵ is not

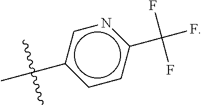

7. A compound of Formula IIIb 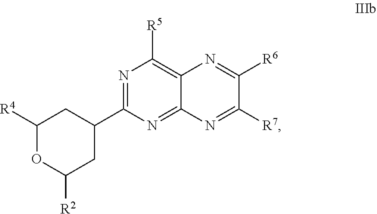
or a pharmaceutically acceptable salt thereof;
wherein
R³ is H or methyl;
R⁴ is 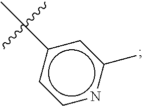
R⁵ is 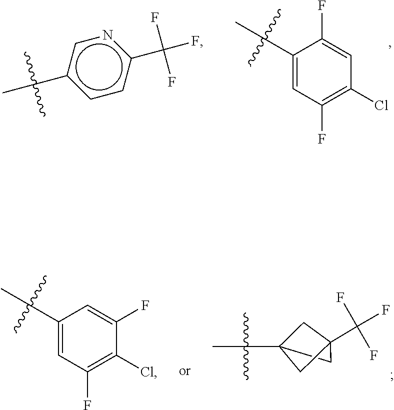
R⁶ is H or methyl; and
R⁷ is methyl.

8. A compound of Formula IIIb 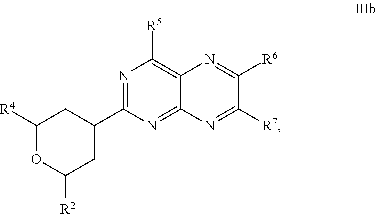
or a pharmaceutically acceptable salt thereof;
wherein
R⁴ is 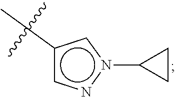
R⁵ is 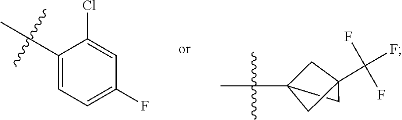 [SMILES: *c1ccc(F)cc1Cl ; "or" ; *C12CC(C(F)(F)F)(C1)C2]
R⁶ is H or methyl; and
R⁷ is Me.

9. A compound of Formula Va 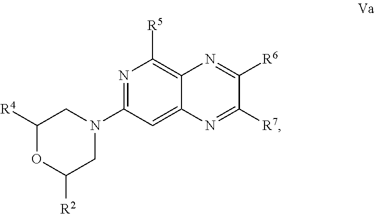
or a pharmaceutically acceptable salt thereof;
wherein
R² is H or methyl;
R⁴ is 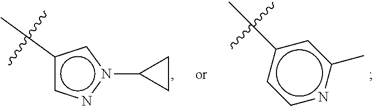
R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that: 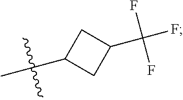 [SMILES: *C1CC(C(F)(F)F)C1]
when R⁶ is Me and R² is H, R⁵ is not
and
when both R² and R⁶ are H, R⁵ is not

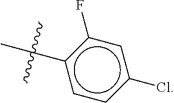

10. A compound of Formula Vb 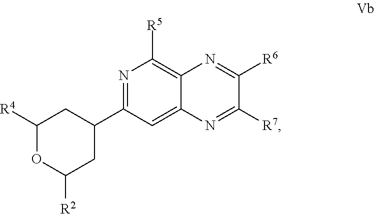
or a pharmaceutically acceptable salt thereof;
wherein
R² is H or methyl;
R⁴ is 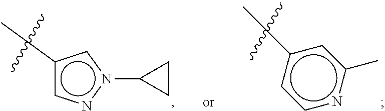
R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl;
provided that when R² is H, R⁵ is not

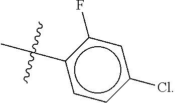

11. A compound of Formula Va or Vb 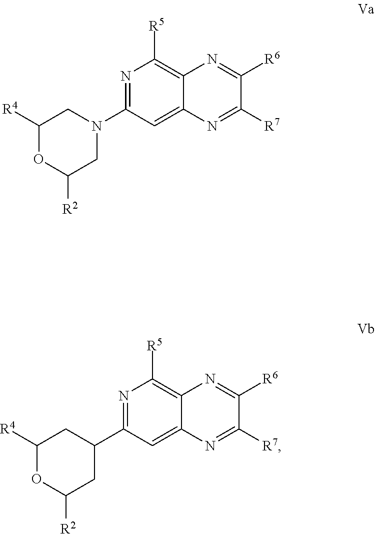
or a pharmaceutically acceptable salt thereof;
wherein
R² is H or methyl;
R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
R⁵ is 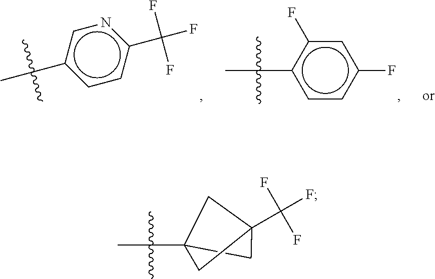
R⁶ is H or methyl; and
R⁷ is methyl.

12. A compound of Formula Va or Vb 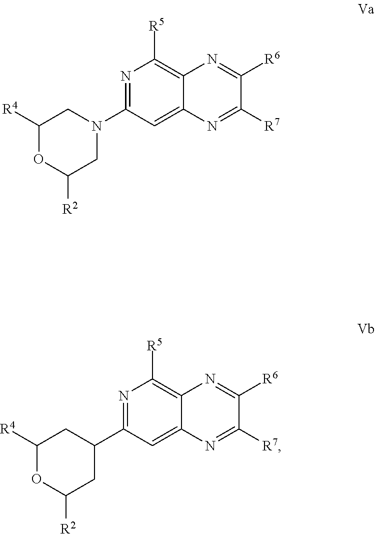
or a pharmaceutically acceptable salt thereof;
wherein
R² is H or methyl;
R⁴ is 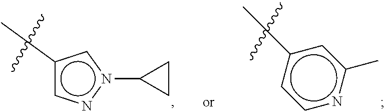
R⁵ is 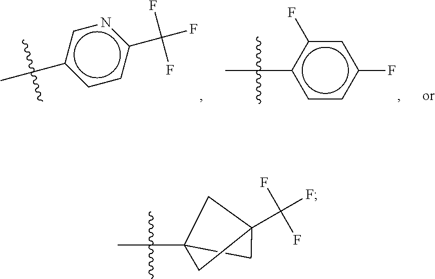
R⁶ is H or methyl; and
R⁷ is methyl.

13. A compound of Formula Va or Vb 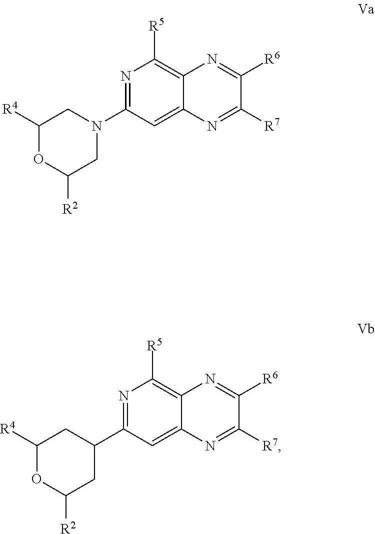
or a pharmaceutically acceptable salt thereof;
wherein
R² is methyl;
R⁴ is 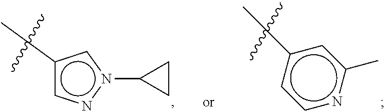
R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl.

14. A compound of Formula Vb 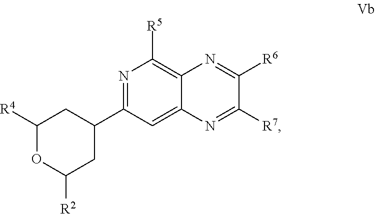
or a pharmaceutically acceptable salt thereof;
wherein
R² is H or methyl;
R⁴ is 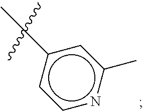
R⁵ is 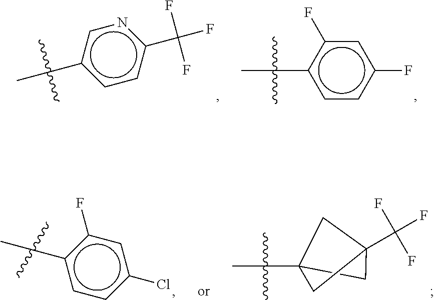
R⁶ is H or methyl; and
R⁷ is methyl;
provided that when R² is H, R⁵ is not

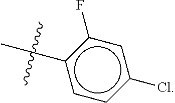

15. A compound of Formula VIIIa 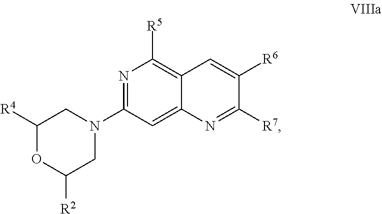
or a pharmaceutically acceptable salt thereof;
wherein
R² is H or methyl;
R⁴ is 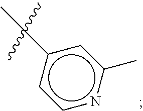
R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is Me
provided that R⁵ is not

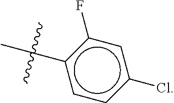

16. A compound of Formula VIIIa 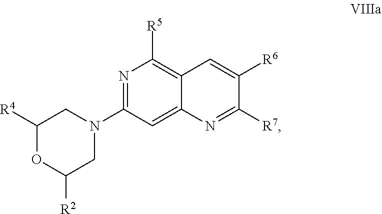
or a pharmaceutically acceptable salt thereof;
wherein
R³ is H or methyl;
R⁴ is 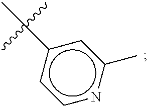
R⁵ is 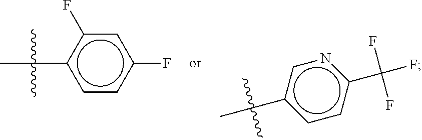
R⁶ is H or methyl; and
R⁷ is methyl.

17. A compound of Formula VIIIb 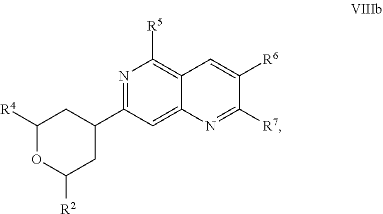
or a pharmaceutically acceptable salt thereof;
wherein
R² is H or methyl;
R⁴ is 5-membered heteroaryl or 6-membered heteroaryl; wherein the 5-membered heteroaryl or 6-membered heteroaryl group is optionally substituted with 1 to 3 substituents independently selected from C₁₋₆alkyl, C₁₋₆alkoxy, and C₃₋₆cycloalkyl;
R⁵ is C₁₋₆haloalkyl, C₃₋₆cycloalkyl, C₅₋₈spiroalkyl, C₅₋₈tricycloalkyl, cyclopent-1-en-1-yl, cyclohex-1-en-1-yl, phenyl, 6-membered heteroaryl, aziridine-1-yl, pyrrolidine-1-yl, 3-azabicyclo[3.1.0]hexan-3-yl, piperidine-1-yl, or —OCH₂—(C₃₋₆cycloalkyl),
wherein the C₃₋₆cycloalkyl, C₅₋₈spiroalkyl, C₅₋₈tricycloalkyl, cyclopent-1-en-1-yl, cyclohex-1-en-1-yl, phenyl, and 6-membered heteroaryl is further optionally substituted with 1 to 4 substituents independently selected from halogen, C₁₋₃alkyl, and C₁₋₃haloalkyl, and
wherein the aziridine-1-yl, pyrrolidine-1-yl, 3-azabicyclo[3.1.0]hexan-3-yl, piperidine-1-yl, and —OCH₂—(C₃₋₆cycloalkyl) is further substituted with 1 to 4 substituents independently selected from halogen, C₁₋₃alkyl, C₁₋₃haloalkyl, and C₁₋₃alkoxy;
R⁶ is H or methyl; and
R⁷ is methyl.

18. A compound of Table A or A-2, or a pharmaceutically acceptable salt thereof.

19. A pharmaceutical composition comprising the compound according to any one of claims 1-18, or a tautomer thereof, or a pharmaceutically acceptable salt of said compound or said tautomer, and a pharmaceutically acceptable excipient.

20. A compound according to any one of claims 1-18, or a tautomer thereof, or a pharmaceutically acceptable salt of said compound or said tautomer, or the pharmaceutical composition according to claim 19 for use as a medicament.

21. A compound according to any one of claims 1-18, or a tautomer thereof, or a pharmaceutically acceptable salt of said compound or said tautomer, or the pharmaceutical composition according to claim 19 for use in treating or preventing a condition associated with a loss of function of human TREM2.

22. A compound according to any one of claims 1-18, or a tautomer thereof, or a pharmaceutically acceptable salt of said compound or said tautomer, or the pharmaceutical composition according to claim 19 for use in treating or preventing Parkinson's disease, rheumatoid arthritis, Alzheimer's disease, Nasu-Hakola disease, frontotemporal dementia, multiple sclerosis, prion disease, or stroke.

23. Use of the compound according to any one of claims 1-18, or a tautomer thereof, or a pharmaceutically acceptable salt of said compound or said tautomer, or the pharmaceutical composition according to claim 19 in the preparation of a medicament for treating or preventing a condition associated with a loss of function of human TREM2.

24. Use of the compound according to any one of claims 1-18, or a tautomer thereof, or a pharmaceutically acceptable salt of said compound or said tautomer, or the pharmaceutical composition according to claim 19 in the preparation of a medicament for treating or preventing Parkinson's disease, rheumatoid arthritis, Alzheimer's disease, Nasu-Hakola disease, frontotemporal dementia, multiple sclerosis, prion disease, or stroke.

25. A method of treating or preventing a condition associated with a loss of function of human TREM2 in a subject in need thereof, the method comprising administering to the subject a therapeutically effective amount of the compound according to any one of claims 1-18, or a tautomer thereof, or a pharmaceutically acceptable salt of said compound or said tautomer.

26. A method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound according to any one of claims 1-18, or a tautomer thereof, or a pharmaceutically acceptable salt of said compound or said tautomer.
